(19) United States
(12) Patent Application Publication
(10) Pub. No.: US 2025/0345443 A1
(43) Pub. Date: Nov. 13, 2025

(54) PHARMACEUTICAL COMPOUND AND USE THEREOF

(71) Applicant: DUALITY BIOLOGICS (SUZHOU) CO., LTD., Suzhou (CN)

(72) Inventors: Yu ZHANG, Shanghai (CN); Bing LI, Shanghai (CN); Xi LI, Shanghai (CN); Haiqing HUA, Shanghai (CN); Zhongyuan ZHU, Shanghai (CN)

(21) Appl. No.: 18/871,011

(22) PCT Filed: Jun. 2, 2023
(86) PCT No.: PCT/CN2023/098069

§ 371 Date: Dec. 2, 2024

(30) Foreign Application Priority Data
Jun. 2, 2022 (CN) 202210624727.1

(51) Int. Cl. A61K 47/68; A61K 47/65; A61P 35/00

(52) U.S. Cl. CPC A61K 47/6801; A61K 47/65; A61K 47/6849; A61K 47/6889; A61P 35/00

(57) ABSTRACT

The present invention relates to a compound or a ligand-drug conjugate thereof, or an isomer, a mixture form, or a pharmaceutically acceptable salt thereof. The present invention further relates to a preparation method for and the use of the compound or the ligand-drug conjugate.

### TECHNICAL FIELD

The present application relates to the field of biomedicine, and particularly to a pharmaceutical compound and a ligand-drug conjugate, as well as use thereof.

### BACKGROUND

The highly conserved pattern recognition receptor protein Toll-like receptor (TLR) family is believed to be involved in innate immunity as receptors of pathogen-associated molecular patterns (PAMPs).

Related compounds that influence TLR activity may affect therapies for diseases including autoimmunity, inflammation, allergy, asthma, transplant rejection, graft-versus-host disease, infection, cancer, or immunodeficiency. There is therefore an urgent need in the art for compounds or drug conjugates capable of influencing TLR activity.

### SUMMARY

The present application provides a compound or a ligand-drug conjugate thereof, or an isomer thereof, or a mixture thereof, or a pharmaceutically acceptable salt thereof, wherein the compound or the ligand-drug conjugate may have an effect selected from the group consisting of: inhibiting tumor growth, influencing Toll-like receptor (TLR) functionality, and influencing immune system functionality.

In one aspect, the present application provides a compound or a conjugate thereof, or an isomer thereof, or a mixture thereof, or a pharmaceutically acceptable salt thereof, wherein the compound has a structure represented by formula (II-a):

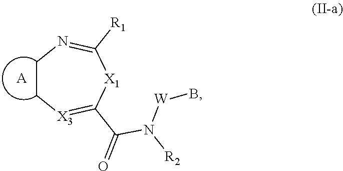

wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
B is substituted with one or more optionally substituted amino groups, wherein the one or more amino groups are each independently substituted with hydrogen or optionally substituted C₁-C₆ alkyl;
ring A is selected from: optionally substituted alcyl, optionally substituted aliphatic heterocyclyl, optionally substituted aryl, and optionally substituted heteroaryl;
ring A may be substituted with m R_{A-1}, wherein each R_{A-1} is independently selected from any group; m is selected from 1, 2, 3, 4, or 5.

In another aspect, the present application provides a ligand-drug conjugate, or an isomer thereof, or a mixture thereof, or
a pharmaceutically acceptable salt thereof, wherein the ligand-drug conjugate comprises a structure represented by formula (II-2A):

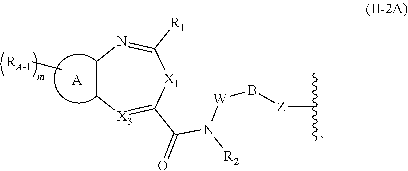

wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl; ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl; when R_{A-1} comprises methylene units, the methylene units of R_{A-1} are each independently unreplaced, or are each independently replaced by any structure;
m is selected from 1, 2, or 3;
the wavy line [symbol] in the general formula represents being directly linked to a ligand via the nitrogen atom on the Z group, or being linked to the ligand via a linker unit.

In some embodiments, in the ligand-drug conjugate, or the isomer thereof, or the mixture thereof, or the pharmaceutically acceptable salt thereof described herein, the ligand-drug conjugate comprises a structure represented by formula (II-2B):

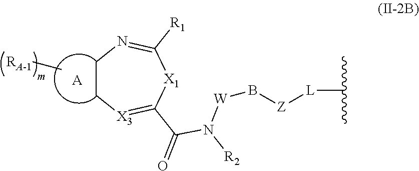

wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl;
ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl; when R_{A-1} comprises methylene units, the methylene units of R_{A-1} are each independently unreplaced, or are each independently replaced by any structure;
m is selected from 1, 2, or 3;
the wavy line [symbol] in the general formula represents being linked to the ligand via the nitrogen atom or the carbon atom on the L group;
L is a linker unit.

In some embodiments, in the ligand-drug conjugate, or the isomer thereof, or the mixture thereof, or the pharmaceutically acceptable salt thereof described herein, the ligand-drug conjugate has a structure represented by formula (II-2C):

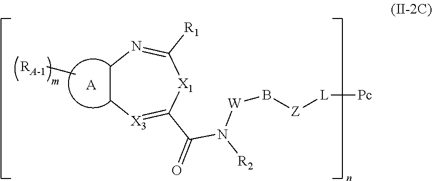

wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl; ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl;
when R_{A-1} comprises methylene units, the methylene units of R_{A-1} are each independently unreplaced, or are each independently replaced by any structure;
m is selected from 1, 2, or 3;
L is a linker unit;
n is an integer or a decimal from 1 to 10;
Pc is a ligand.

In certain preferred embodiments of the present disclosure, certain groups in the structures represented by formulas (II-a), (II-2A), (II-2B) and (II-2C) are defined as follows, and groups not mentioned are as described in any one of the embodiments of the present application.

In some embodiments, in the structures represented by formulas (II-a), (II-2A), (II-2B) and (II-2C), R₁ is —NH₂.

In some embodiments, in the structures represented by formulas (II-a), (II-2A), (II-2B) and (II-2C), X₁ is selected from —CH₂— or

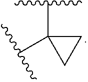 [SMILES: *C1(*)CC1]

In some embodiments, in the structures represented by formulas (II-a), (II-2A), (II-2B) and (II-2C), X₃ is selected from —CH═ or —C(CH₃)=.

In some embodiments, in the structures represented by formulas (II-a), (II-2A), (II-2B) and (II-2C), R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂-2, each R₂₋₂ being independently selected from: hydrogen, halogen, hydroxy, or amino; the methylene units of R₂ are each independently unreplaced, replaced by —O—, or replaced by —N(R₂₋₃)—, R₂₋₃ being selected from: hydrogen or C₁-C₃ alkyl.

In some embodiments, in the structures represented by formulas (II-a), (II-2A), (II-2B) and (II-2C), each R₂₋₁ is independently selected from: hydrogen, fluorine, chlorine, bromine, iodine, hydroxy, amino, —CN, —CH₃, —CH₂F, —CHF₂, —CF₃, —CH₂CH₃, or

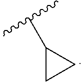 [SMILES: *C1CC1]

In some embodiments, in the structures represented by formulas (II-a), (II-2A), (II-2B) and (II-2C), R₂ is selected from

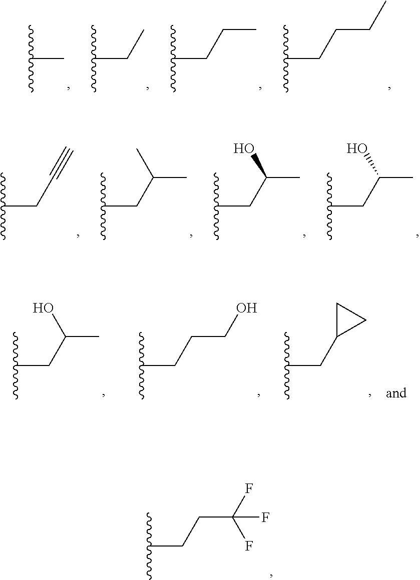

wherein the methylene units of R₂ are each independently unreplaced, replaced by —O—, or replaced by —NH—.

In some embodiments, in the structures represented by formulas (II-a), (II-2A), (II-2B) and (II-2C), R₂ is selected from:

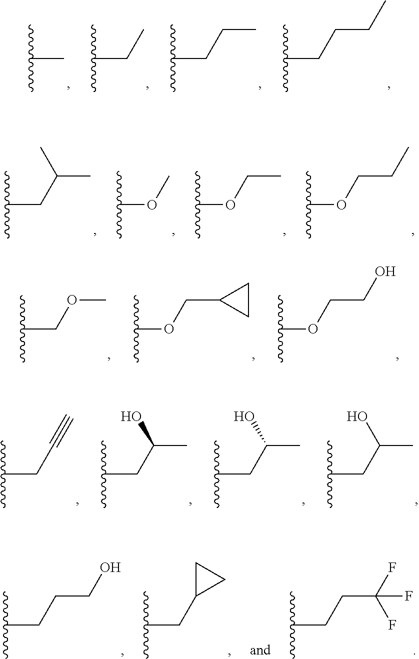

For example, R₂ is selected from

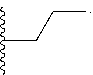 [SMILES: *CCC]

In some embodiments, in the structures represented by formulas (II-a), (II-2A), (II-2B) and (II-2C), W is selected from C₁-C₆ alkyl optionally substituted with 1, 2, or 3 R_{w-1}; each R_{w-1} is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2}, each R_{w-2} being independently selected from: hydrogen, halogen, hydroxy, or amino;
the methylene units of W are each independently unreplaced, replaced by —O—, or replaced by —N(R_{w-3})—; R_{w-3} is selected from: hydrogen or C₁-C₃ alkyl.

In some embodiments, in the structures represented by formulas (II-a), (II-2A), (II-2B) and (II-2C), each R_{w-1} is independently selected from: hydrogen, fluorine, chlorine, bromine, iodine, hydroxy, amino, —CN, —CH₃, —CH₂F, —CHF₂, —CF₃, —CH₂CH₃, or

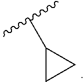 [SMILES: *C1CC1]

In some embodiments, in the structures represented by formulas (II-a), (II-2A), (II-2B) and (II-2C), W is selected from

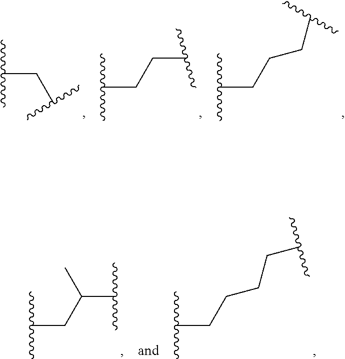

wherein the methylene units of W are each independently unreplaced, replaced by —O—, or replaced by —NH—.

In some embodiments, in the structures represented by formulas (II-a), (II-2A), (II-2B) and (II-2C), W is selected from

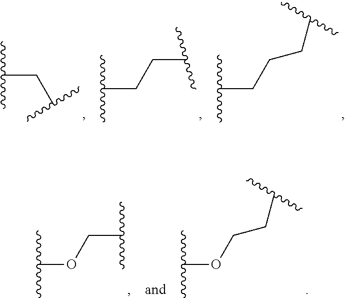

For example, W is selected from or

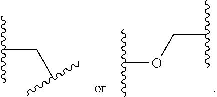

In some embodiments, in the structures represented by formulas (II-a), (II-2A), (II-2B) and (II-2C), B is selected from phenyl and pyridinyl, wherein the phenyl and the pyridinyl are each independently optionally substituted with 1, 2, or 3 R_{B-1}, each R_{B-1} being independently selected from hydrogen, halogen, hydroxy, amino, C₁-C₃ alkyl, and C₁-C₃ alkoxy.

In some embodiments, in the structures represented by formulas (II-a), (II-2A), (II-2B) and (II-2C), B is selected from

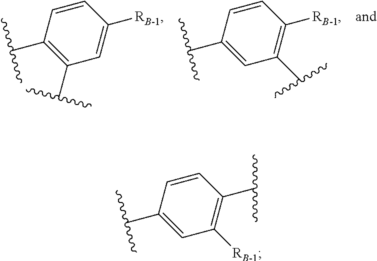

preferably, B is selected from

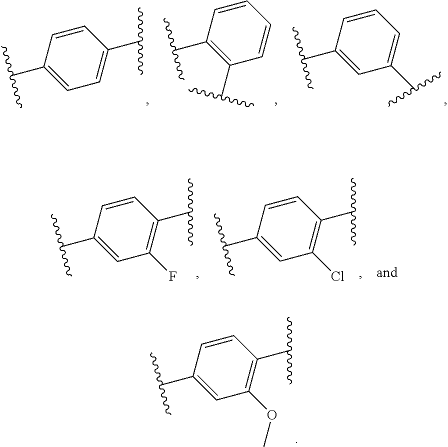

For example, B is selected from

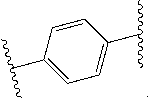 [SMILES: *c1ccc(*)cc1]

In some embodiments, in the structures represented by formulas (II-a), (II-2A), (II-2B) and (II-2C), A is selected from: phenyl, pyridinyl, pyrrolyl, thienyl, furanyl, pyridazinyl, pyrimidinyl, and pyrazinyl.

In some embodiments, in the structures represented by formulas (II-a), (II-2A), (II-2B) and (II-2C), A is selected from phenyl and pyridinyl. For example, ring A is selected from phenyl. For example, ring A is selected from pyridinyl.

In some embodiments, in the structures represented by formulas (II-a), (II-2A), (II-2B) and (II-2C), R_{A-1} is R_{A2-1}-Lx-;

Lx is selected from: —(CH₂)ₜ—, —(CH₂)ₜ₀—, —O(CH₂)ₜ—, —(CH₂)ₜN(R_{L-1})—, —(CH₂)ₜS—, —(CH₂)ₜC(═O)—, —(CH₂)ₜN(R_{L-1})C(═O)—,

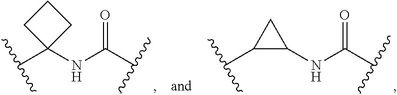 [SMILES: *C(=O)NC1(*)CCC1 ; *C(=O)NC1CC1*]

wherein R_{L-1} is selected from H or C₁-C₃ alkyl, and t is selected from 0, 1, 2, or 3;
R_{A2-1} is selected from hydrogen, halogen, hydroxy, amino, cyano, C₁-C₆ alkyl, alcyl, aliphatic heterocyclyl, aryl, and heteroaryl, wherein the C₁-C₆ alkyl, the alcyl, the aliphatic heterocyclyl, the aryl, and the heteroaryl are each independently optionally substituted with 1, 2, or 3 R_{A2-2}; R_{A2-2} is selected from hydrogen, halogen, hydroxy, amino, and C₁-C₆ alkyl optionally substituted with 1, 2, or 3 R_{A2-3}; each R_{A2-3} is independently selected from: hydrogen, halogen, hydroxy, amino, and C₁-C₃ alkyl;
any methylene unit of R_{A2-1} may be replaced by the following structure: —O—, —S—, —S(═O)₂—, —NH—, —CO—,

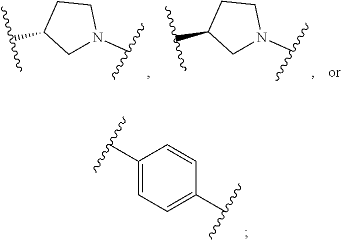 [SMILES: *[C@@H]1CCN(*)C1 ; *[C@H]1CCN(*)C1 ; *c1ccc(*)cc1]

m is selected from 1, 2, or 3.

In some embodiments, in the structures represented by formulas (II-a), (II-2A), (II-2B) and (II-2C), R_{A-1}
is R_{A2-1}-Lx-; Lx is a single bond or —NHC(═O)—.

In some embodiments, in the structures represented by formulas (II-a), (II-2A), (II-2B) and (II-2C), R_{A-1}
is R_{A2-1}-Lx-; each R_{A2-1} is independently selected from hydrogen, halogen, hydroxy, amino, cyano, or C₁-C₆ alkyl,

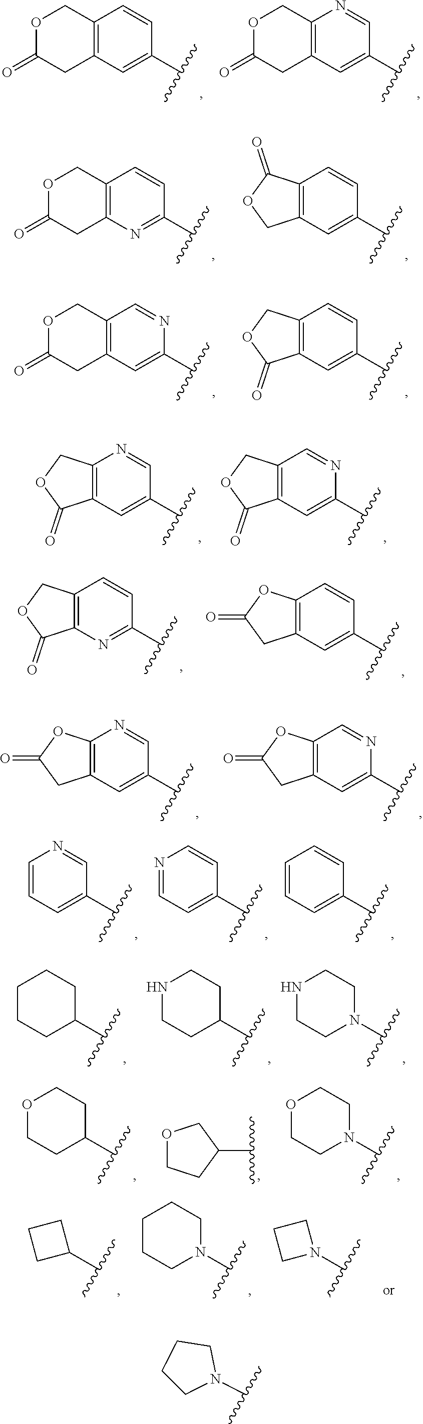

optionally substituted with 1, 2, or 3 R_{A2-2}, wherein any methylene unit of R_{A2-1} may be replaced by the following structure: —O—, —S—, —S(═O)—, —S(═O)₂—, —NH—, —C(O)—,

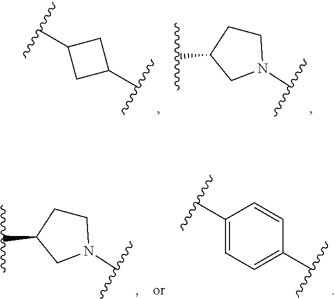 [SMILES: *C1CC(*)C1 ; *[C@@H]1CCN(*)C1 ; *[C@H]1CCN(*)C1 ; *c1ccc(*)cc1]

In some embodiments, in the structures represented by formulas (II-a), (II-2A), (II-2B) and (II-2C), R_{A-1} is selected from: hydrogen, fluorine, chlorine, bromine, iodine, hydroxy, amino, cyano, sulfhydryl, —CH₃, —CH(═O), —C(═O)OH, —OCH(═O), —SH(═O), —SH(═O)₂, —CH₂CH₃, —CH₂OH, —OCH₃, —NHCH₃, —CH₂NH₂, —S(═O)₂NH₂, —NHSH(═O)₂, —SCH₃, —CH₂SH, —S(═O)CH₃, —CH₂SH(═O), —S(═O)₂CH₃, —CH₂SH(═O)₂, —NHCH(═O), —C(═O)NH₂, —CH₂CH(═O), —CH₂NHCH(═O), —C(═O)NHCH₃,

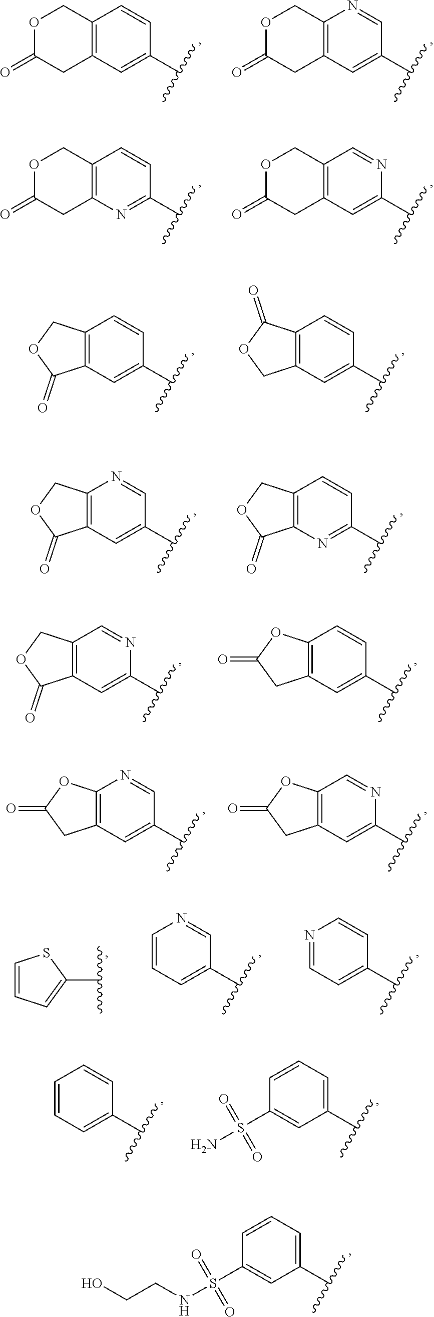

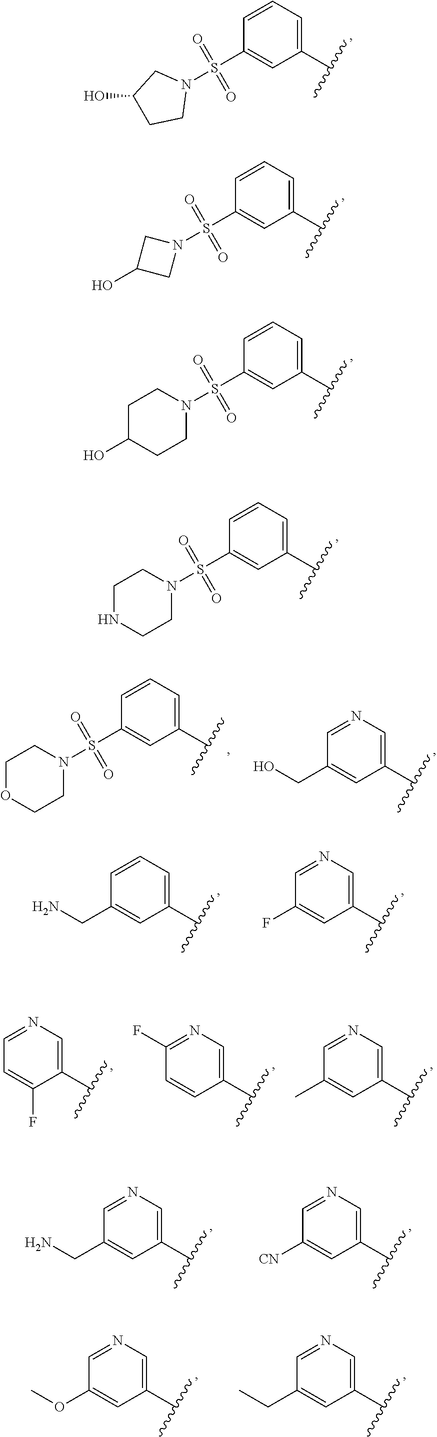

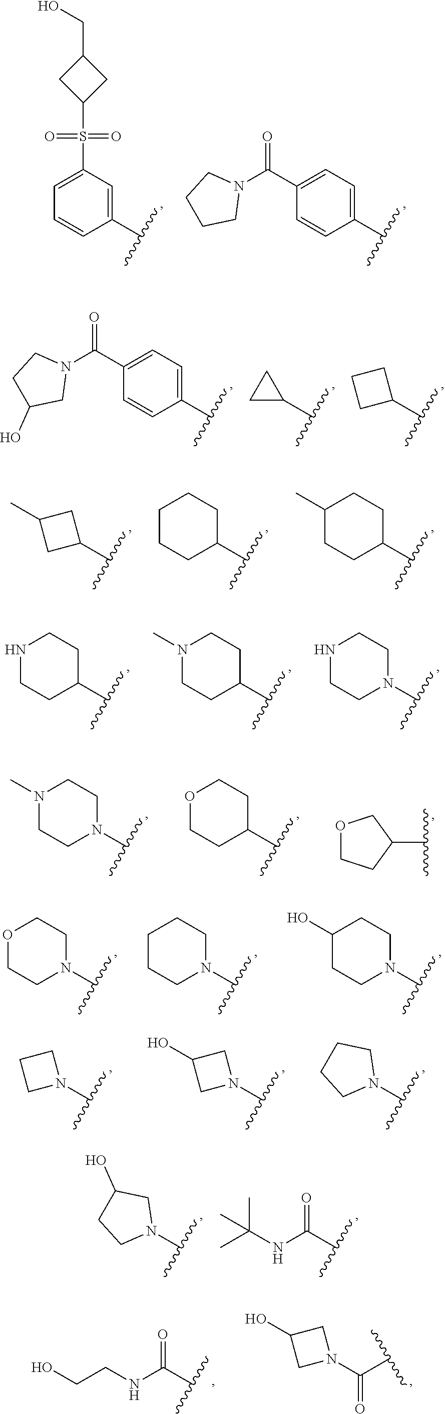

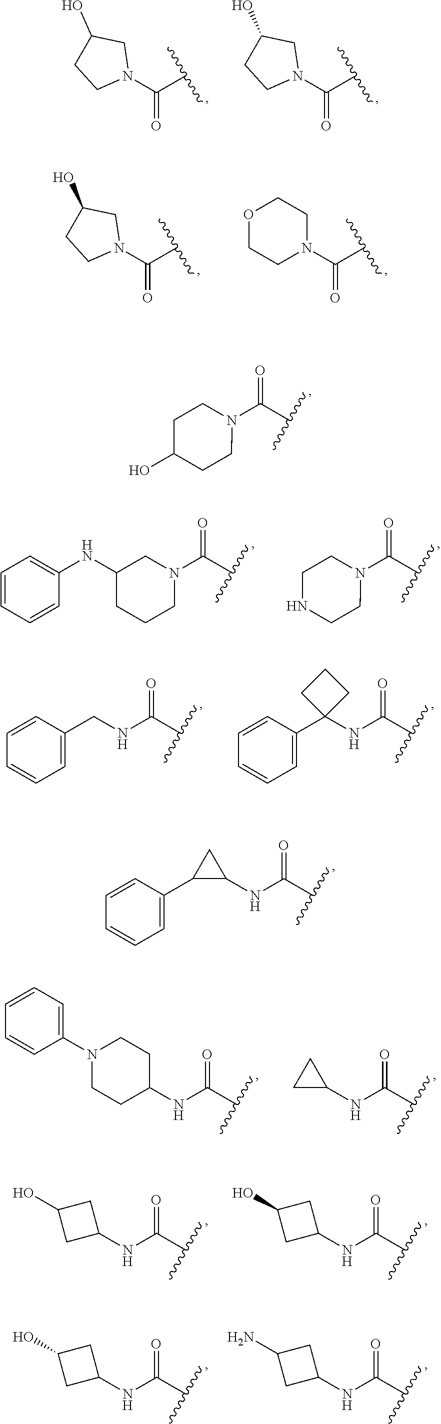

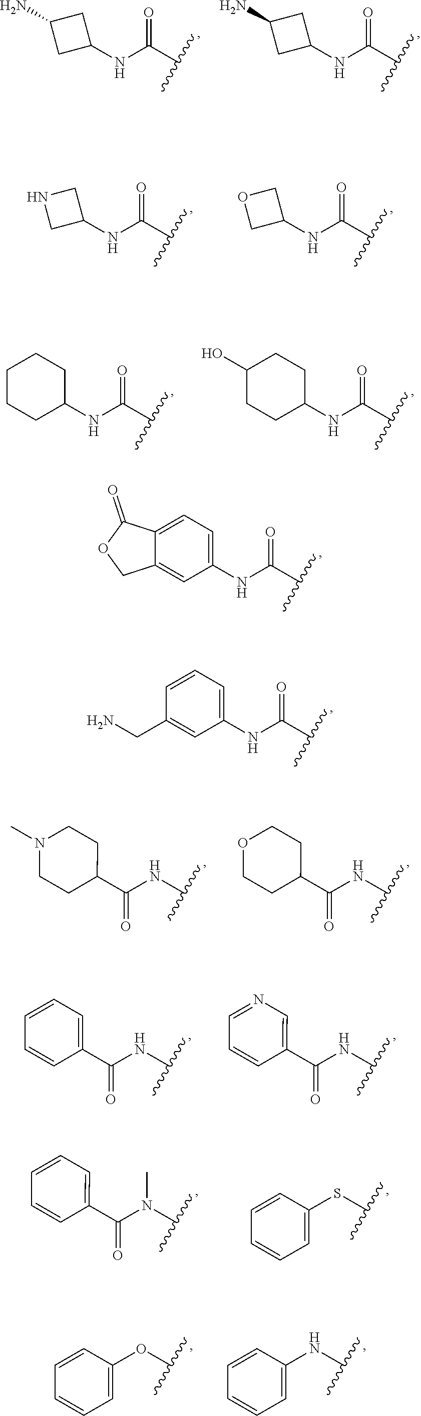

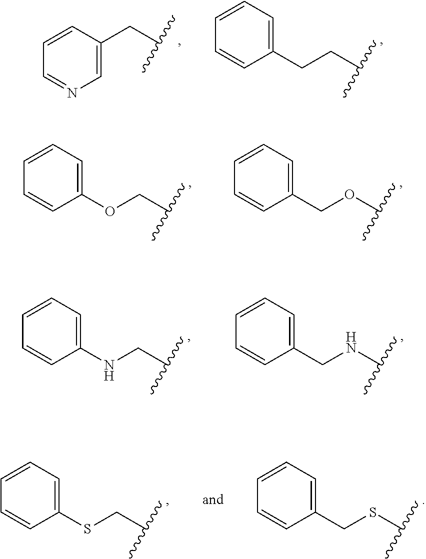

In some embodiments, in the structures represented by formulas (II-a), (II-2A), (II-2B) and (II-2C), R_{A-1} is R_{A2-1}-Lx-, wherein Lx is a single bond, and each R_{A2-1} is independently selected from phenyl and pyridinyl optionally substituted with 1, 2, or 3 R_{A2-2}; R_{A2-2} is selected from hydrogen, halogen, hydroxy, amino, and C₁-C₆ alkyl optionally substituted with 1, 2, or 3 R_{A2-3}; each R_{A2-3} is independently selected from: hydrogen, halogen, hydroxy, and amino;
preferably, R_{2A-1} is selected from:

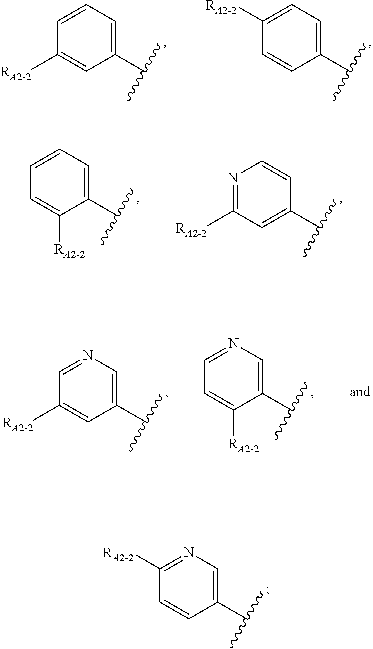

more preferably, R_{A-1} is selected from:

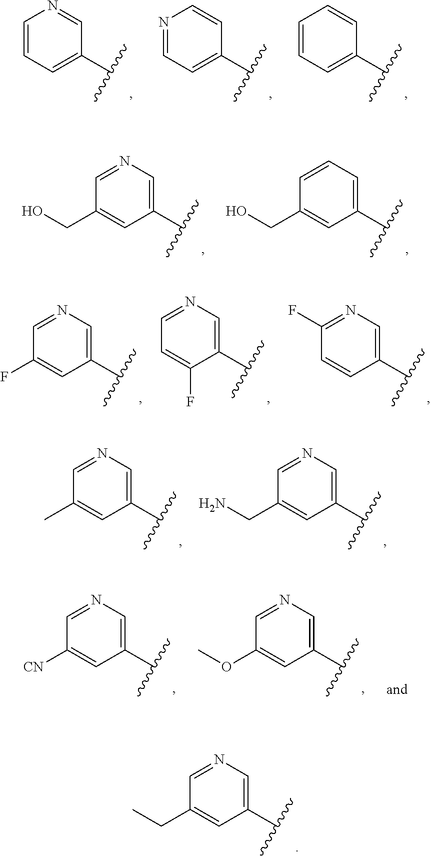

In some embodiments, in the structures represented by formulas (II-a), (II-2A), (II-2B) and (II-2C), R_{A-1} is
R_{A2-1}-Lx-, wherein Lx is a single bond, and R_{A2-1} is selected from

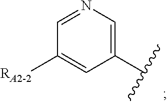

preferably, R_{A2-2} is —CH₂OH.

In some embodiments, in the structures represented by formulas (II-a), (II-2A), (II-2B) and (II-2C), R_{A-1} is R_{A2-1}-Lx-, wherein Lx is —NHC(═O)—, and each R_{A2-1} is independently selected from phenyl and pyridinyl optionally substituted with 1, 2, or 3 R_{A2-2}; R_{A2-2} is selected from hydrogen, halogen, hydroxy, amino, and C₁-C₆ alkyl optionally substituted with 1, 2, or 3 R_{A2-3}; each R_{A2-3} is independently selected from: hydrogen, halogen, hydroxy, and amino;
preferably, R_{2A-1} is selected from:

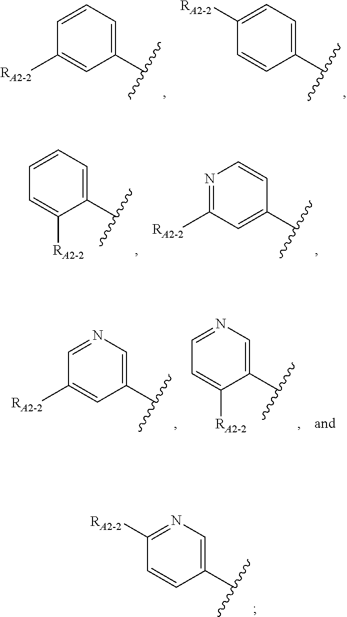

more preferably, R_{2A-1} is selected from:

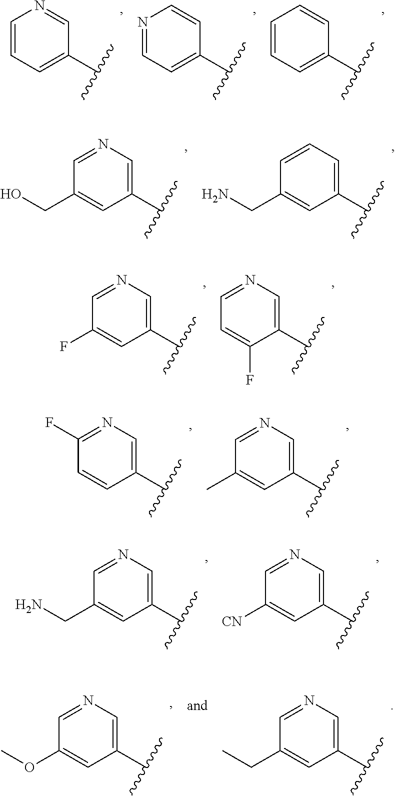

In some embodiments, in the structures represented by formulas (II-2A), (II-2B) and (II-2C), Z is selected from —N(R_{Z-1})—, wherein R_{Z-1} is selected from H or C₁-C₆ alkyl optionally substituted with 1, 2, or 3 fluorine, chlorine, bromine, iodine, hydroxy, or amino substituents; preferably, R_{Z-1} is selected from H, —CH₃, —CH₂F, —CHF₂, —CF₃, and —CH₂CH₃; more preferably, R_{Z-1} is selected from H.

In some embodiments, in the structures represented by formulas (II-2A), (II-2B) and (II-2C), the structural unit

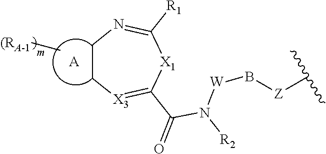

is selected from:

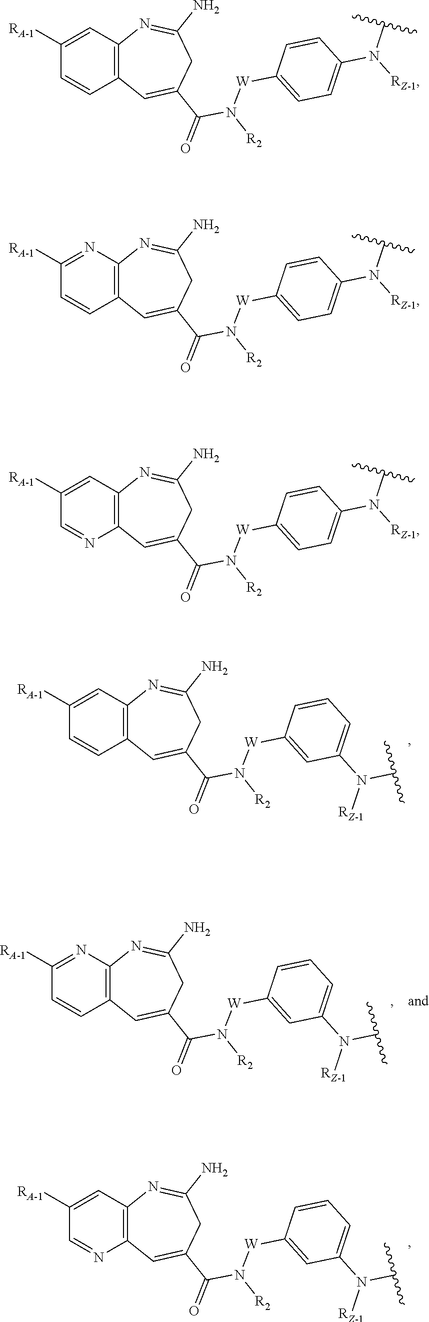

wherein
R_{Z-1}, R₂, W, and R_{A-1} are as described in any one of the embodiments of (II-2A), (II-2B), and (II-2C) of the present application.

In some embodiments, the present application provides a compound or a tautomer, a mesomer, a racemate, an enantiomer or a diastereoisomer thereof, or a mixture thereof, or a pharmaceutically acceptable salt thereof, wherein the compound may comprise a structure represented by formula (II-a):

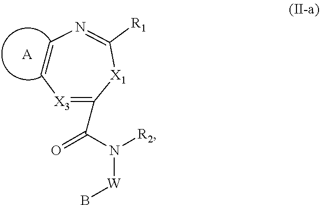

wherein
R₁ may be optionally substituted amino; Xi may be selected from optionally substituted —CH₂—; X₃ may be selected from optionally substituted —CH— or —N—;
R₂ may be selected from the group consisting of: optionally substituted

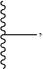

optionally substituted

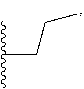 [SMILES: *CCC]

optionally substituted

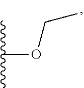 [SMILES: *OCC]

and optionally substituted

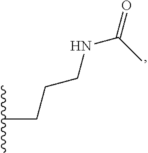 [SMILES: *CCCNC(C)=O]

and each R₂ may independently be substituted with one or more R₂₋₁, wherein the one or more R₂₋₁ may be selected from the group consisting of: hydrogen, fluorine, optionally substituted methyl, optionally substituted cyclopropyl, and optionally substituted phenyl;
W may be selected from the group consisting of: optionally substituted

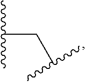 [SMILES: *C*]

optionally substituted

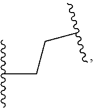 [SMILES: *CC*]

optionally substituted

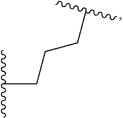 [SMILES: *CCC*]

optionally substituted

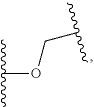 [SMILES: *CO*]

and optionally substituted

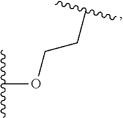 [SMILES: *CCO*]

B may be selected from the group consisting of: optionally substituted phenyl, optionally substituted naphthyl, optionally substituted pyridinyl, optionally substituted pyrrolyl, optionally substituted thienyl, optionally substituted furanyl, and optionally substituted pyrazinyl, and B may be substituted with one or more optionally substituted amino groups;
A may be selected from the group consisting of: optionally substituted phenyl, optionally substituted pyridinyl, optionally substituted pyrrolyl, optionally substituted thienyl, optionally substituted furanyl, and optionally substituted pyrazinyl, and A may be substituted with one or more R_{A-1}, wherein R_{A-1} is absent or may comprise: optionally substituted CH₃—, optionally substituted CH(═CH₂)—, optionally substituted HC(═O)—, optionally substituted HOC(═O)—, optionally substituted HC(═O)O—, optionally substituted NH₂—, optionally substituted HO—, optionally substituted HS—, optionally substituted HS(═O)—, optionally substituted HS(═O)₂—, optionally substituted CH₃CH₂—, optionally substituted CH₂═CH—, optionally substituted HC—C—, optionally substituted HOCH₂—, optionally substituted CH₃O—, optionally substituted CH₃NH—, optionally substituted NH₂CH₂—, optionally substituted HS(═O)₂—NH—, optionally substituted NH₂—S(═O)₂—, optionally substituted HS—CH₂—, optionally substituted CH₃S—, optionally substituted HS(═O)—CH₂—, optionally substituted CH₃—S(═O)—, optionally substituted HS(═O)₂—CH₂—, optionally substituted CH₃—S(═O)₂—, optionally substituted NH₂C(═O)—, optionally substituted HC(═O)NH—, optionally substituted HC(═O)CH₂—, optionally substituted HC(═O)NHCH₂—, optionally substituted CH₃NHC(═O)—, optionally substituted

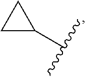 [SMILES: *C1CC1]

optionally substituted

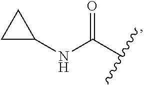 [SMILES: *C(=O)NC1CC1]

optionally substituted

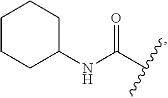 [SMILES: *C(=O)NC1CCCCC1]

optionally substituted

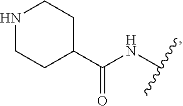 [SMILES: *NC(=O)C1CCNCC1]

optionally substituted

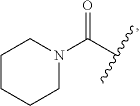 [SMILES: *C(=O)N1CCCCC1]

optionally substituted

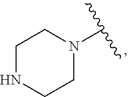 [SMILES: *N1CCNCC1]

optionally substituted

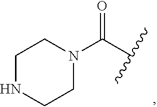 [SMILES: *C(=O)N1CCNCC1]

optionally substituted

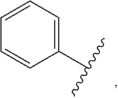 [SMILES: *c1ccccc1]

optionally substituted

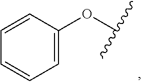 [SMILES: *Oc1ccccc1]

optionally substituted

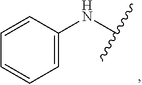 [SMILES: *Nc1ccccc1]

optionally substituted

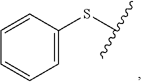 [SMILES: *Sc1ccccc1]

optionally substituted

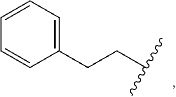 [SMILES: *CCc1ccccc1]

optionally substituted

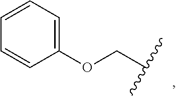 [SMILES: *COc1ccccc1]

optionally substituted

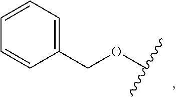 [SMILES: *OCc1ccccc1]

optionally substituted

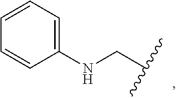 [SMILES: *CNc1ccccc1]

optionally substituted

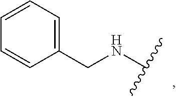 [SMILES: *NCc1ccccc1]

optionally substituted

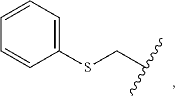 [SMILES: *CSc1ccccc1]

optionally substituted

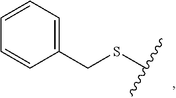 [SMILES: *SCc1ccccc1]

optionally substituted

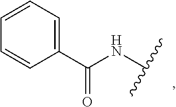 [SMILES: *NC(=O)c1ccccc1]

optionally substituted

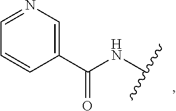 [SMILES: *NC(=O)c1cccnc1]

optionally substituted

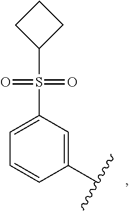 [SMILES: *c1cccc(S(=O)(=O)C2CCC2)c1]

optionally substituted

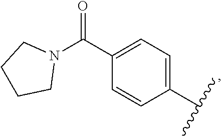 [SMILES: *c1ccc(C(=O)N2CCCC2)cc1]

optionally substituted

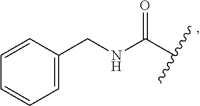 [SMILES: *C(=O)NCc1ccccc1]

optionally substituted

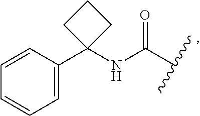 [SMILES: *C(=O)NC1(c2ccccc2)CCC1]

optionally substituted

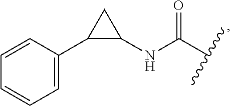 [SMILES: *C(=O)NC1CC1c1ccccc1]

optionally substituted

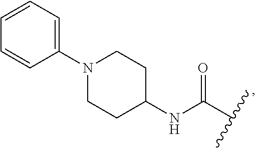 [SMILES: *C(=O)NC1CCN(c2ccccc2)CC1]

optionally substituted

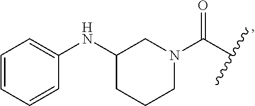 [SMILES: *C(=O)N1CCCC(Nc2ccccc2)C1]

optionally substituted

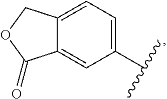 [SMILES: *c1ccc2c(c1)C(=O)OC2]

optionally substituted

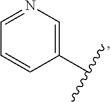 [SMILES: *c1cccnc1]

and/or optionally substituted

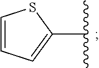 [SMILES: *c1cccs1]

the one or more R_{A-1} may each independently be substituted with one or more R_{A-2}, wherein R_{A-2} may comprise hydrogen, halogen, optionally substituted methyl, and/or optionally substituted hydroxy; the one or more R_{A-2} may each independently be substituted with one or more R_{A-3}, wherein R_{A-3} may comprise optionally substituted methyl and/or optionally substituted hydroxy.

In some embodiments, in the structures represented by formulas (II-2B) and (II-2C), the linker unit L is -Tr-L₃-L₂-L_{1c}-, wherein
Tr is selected from

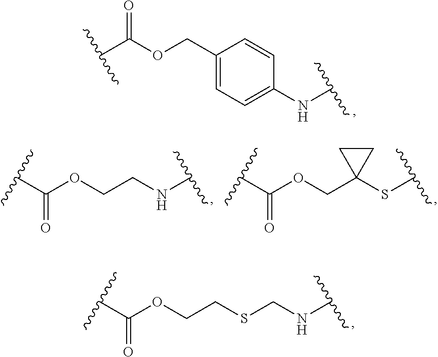 [SMILES: *Nc1ccc(COC(*)=O)cc1 ; *NCCOC(*)=O ; *SC1(COC(*)=O)CC1 ; *NCSCCOC(*)=O]

and
a single bond;
L_{1c} is selected from

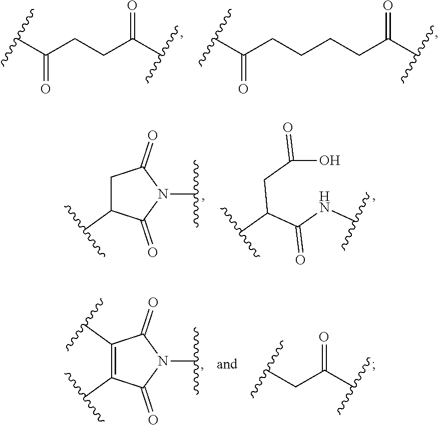 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]

L₂ is selected from

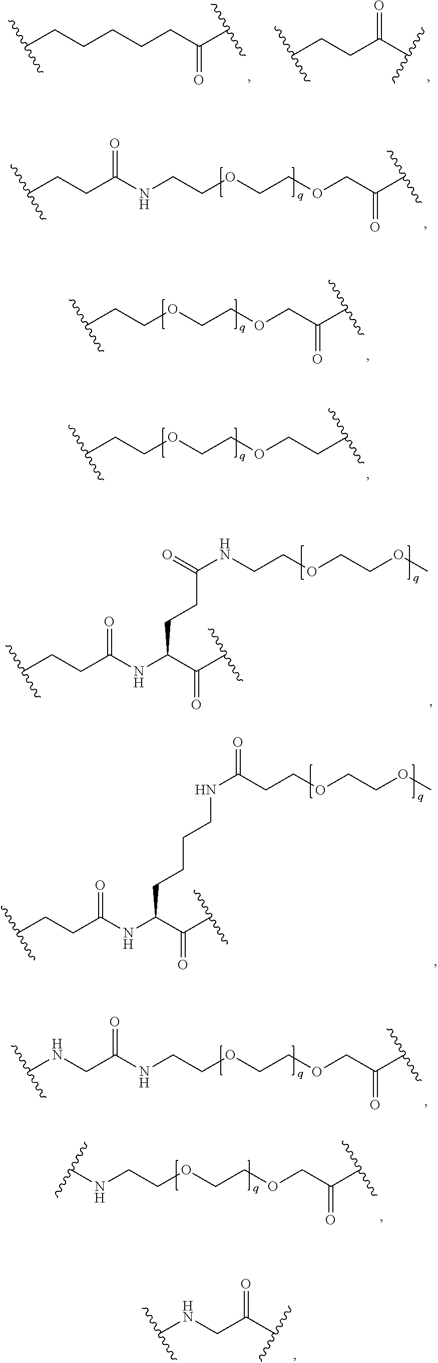

and a single bond, wherein q is an integer from 1 to 20;
L₃ is selected from a single bond or a peptide residue consisting of 1-4 amino acids, wherein the peptide residue is a peptide residue formed by an amino acid selected from the group consisting of: valine, lysine, cysteine, alanine, glycine, glutamic acid, glutamine, phenylalanine, citrulline, aspartic acid, asparagine, and serine.

In some embodiments, in the structures represented by formulas (II-2B) and (II-2C), L₃ is selected from a single bond or the following peptide residues: -valine-cysteine-, -valine-citrulline-, -valine-lysine-,-valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In some embodiments, in the structures represented by formulas (II-2B) and (II-2C), the linker unit L is -L₃-L₂-L_{1c}-;
L_{1c} is selected from

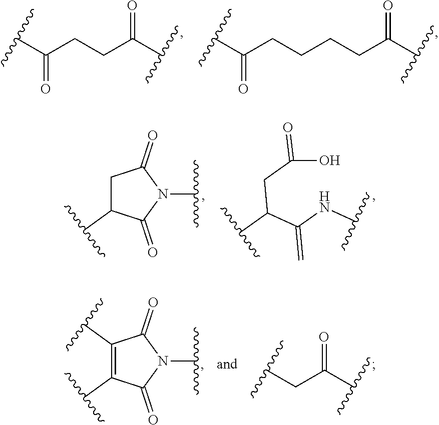

L₂ is selected from

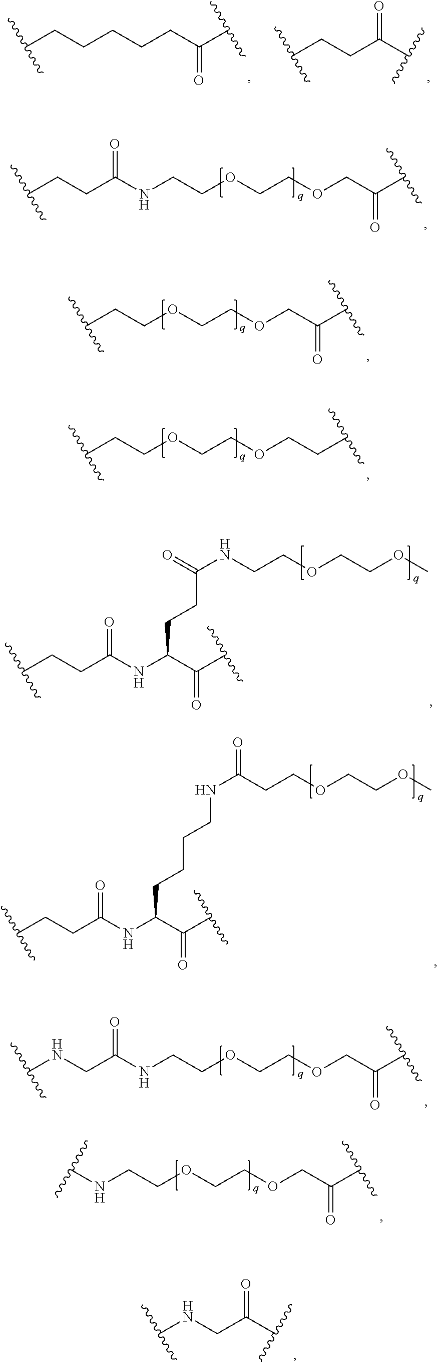

and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In some embodiments, in the structures represented by formulas (II-2B) and (II-2C), the linker unit L is -L₃-L₂-L_{1c}- or -Tr-L₃-L₂-L_{1c}-, wherein the L₃ end or the Tr end is connected to the nitrogen atom of the Z group, and the L_{1c} end is connected to the ligand.

In some embodiments, in the structures represented by formulas (II-2B) and (II-2C), the linker unit L is selected from:

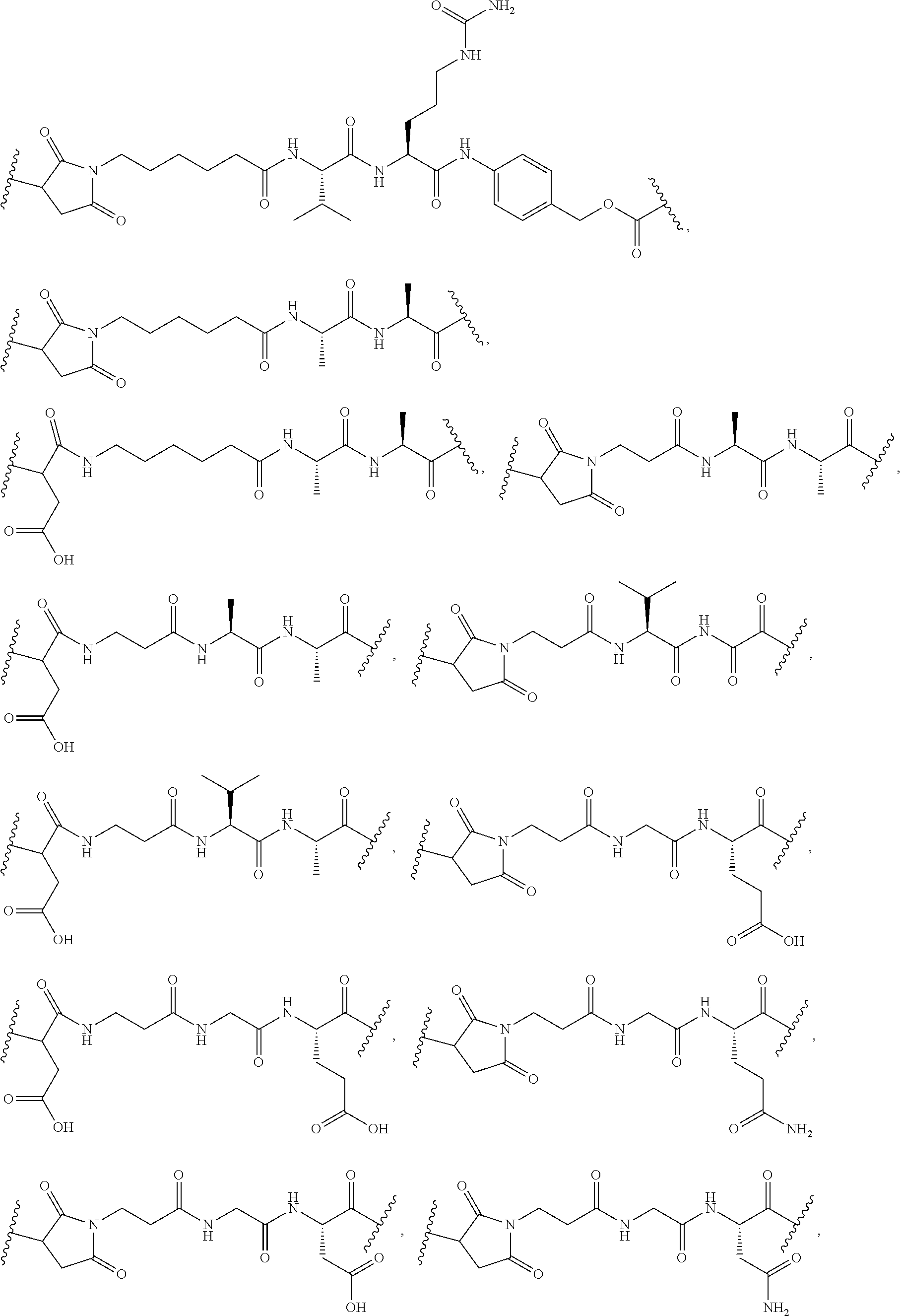

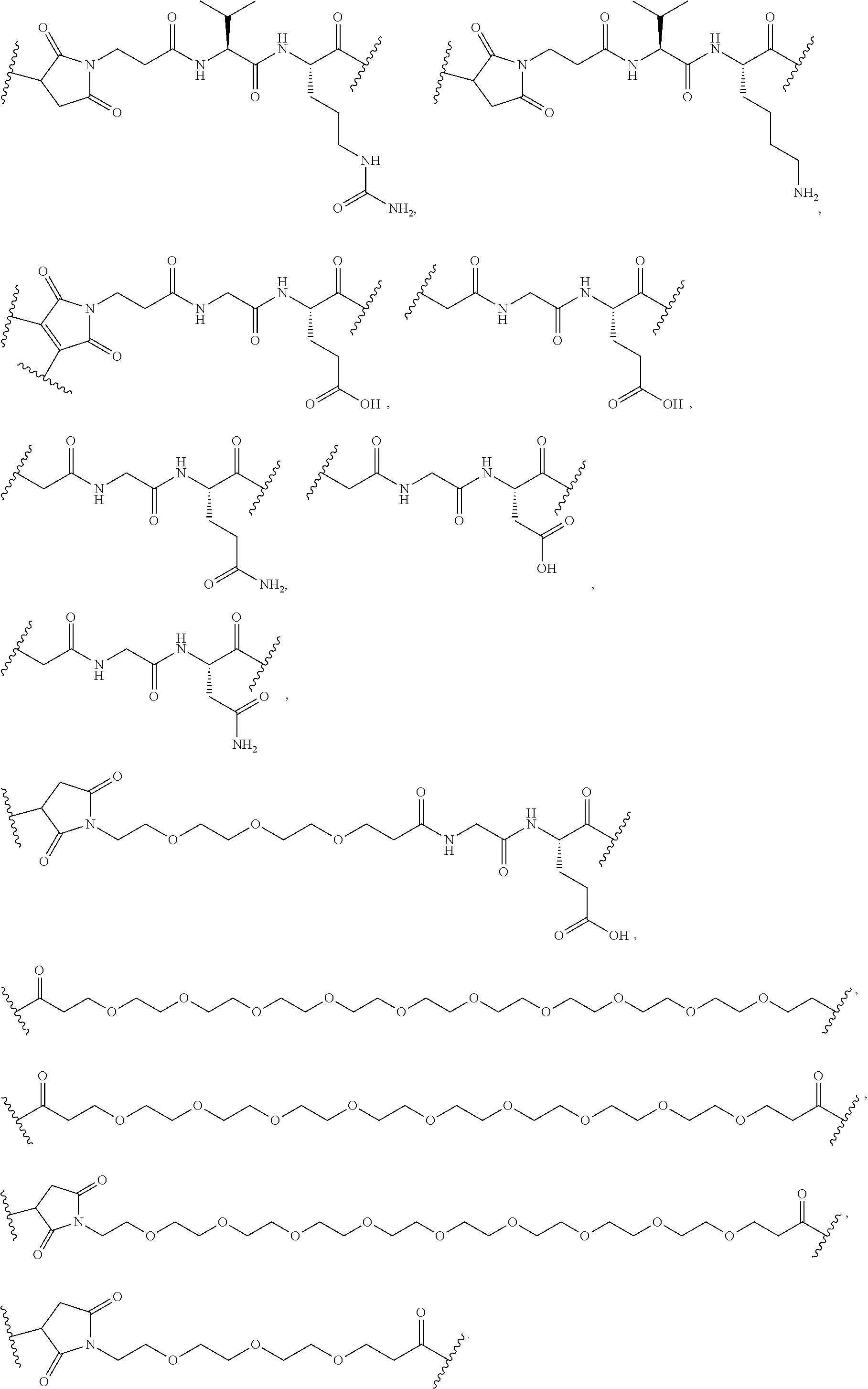

For example, the linker unit L is selected from:

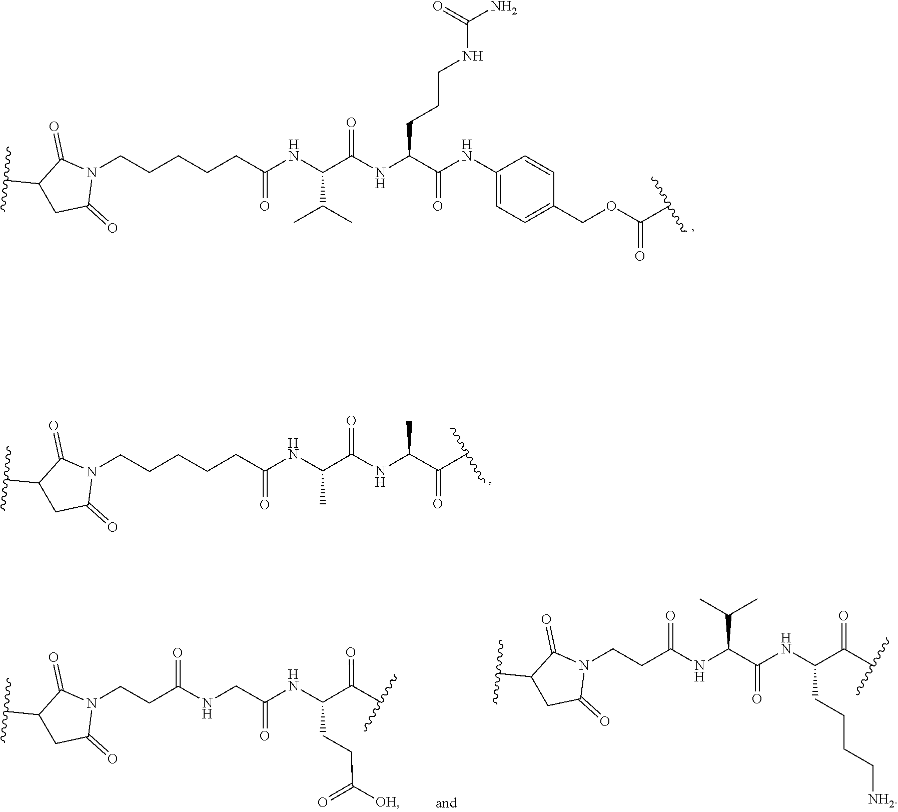 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)[C@H](C)NC(=O)[C@H](C)NC(=O)CCCCCN1C(=O)CC(*)C1=O ; *C(=O)[C@H](CCC(=O)O)NC(=O)CNC(=O)CCN1C(=O)CC(*)C1=O ; *C(=O)[C@H](CCCCN)NC(=O)[C@@H](NC(=O)CCN1C(=O)CC(*)C1=O)C(C)C]

In some embodiments, in the ligand-drug conjugate, or the isomer thereof, or the mixture thereof, or the pharmaceutically acceptable salt thereof, the ligand-drug conjugate is represented by formulas (II-2C-1) and (II-2C-2):

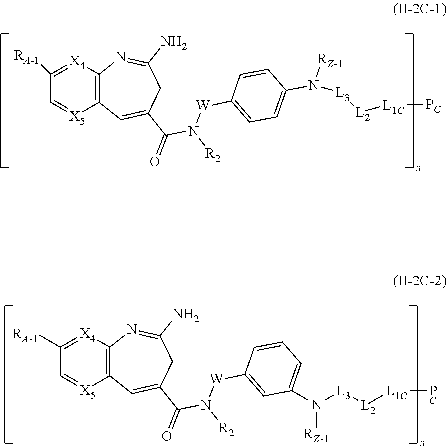

wherein
R_{Z-1} is selected from H or C₁-C₆ alkyl optionally substituted with 1, 2, or 3 fluorine, chlorine, bromine, iodine, hydroxy, or amino substituents;
X₄ and X₅ are each independently selected from —CH═ and —N═;
R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂₋₂; each R₂₋₂ is independently selected from: hydrogen, halogen, hydroxy, or amino; the methylene units of R₂ are each independently unreplaced, replaced by —O—, or replaced by —N(R₂₋₃)—; R₂₋₃ is selected from: hydrogen or C₁-C₃ alkyl;
W is selected from C₁-C₆ alkyl optionally substituted with 1, 2, or 3 R_{w-1}; each R_{w-1} is independently selected from hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2},
each R_{w-2} is independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl;
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein
each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆ alkyl), or —N(C₁-C₆ alkyl)₂;
L_{1c} is selected from

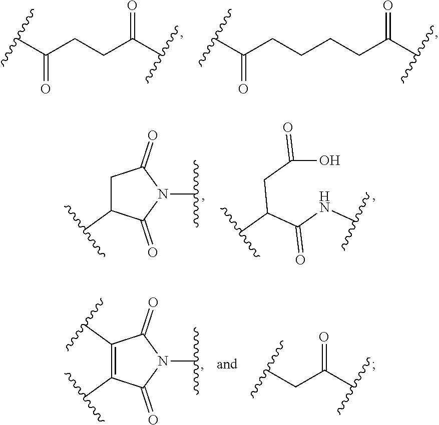 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]

L₂ is selected from

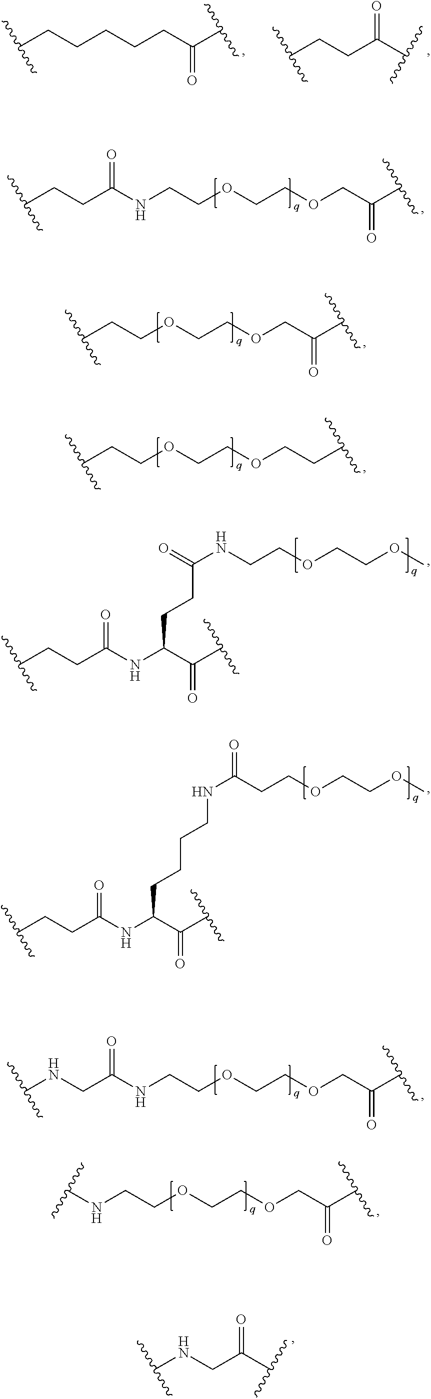

and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-;
n is an integer or a decimal from 1 to 10;
Pc is a ligand.

In certain preferred embodiments of the present disclosure, in the structures represented by formulas (II-2C-1) and (II-2C-2), X₄ and Xₛ are each independently selected from —CH═ and —N═; R_{Z-1}, R₂, W, R_{A-1}, L_{1c}, L₂, L₃, Pc, and n are as described in any one of the embodiments of (II-2C) of the present application.

In some embodiments, in the ligand-drug conjugate, or the isomer thereof, or the mixture thereof, or the pharmaceutically acceptable salt thereof described herein, the ligand-drug conjugate is selected from the following structural formulas:

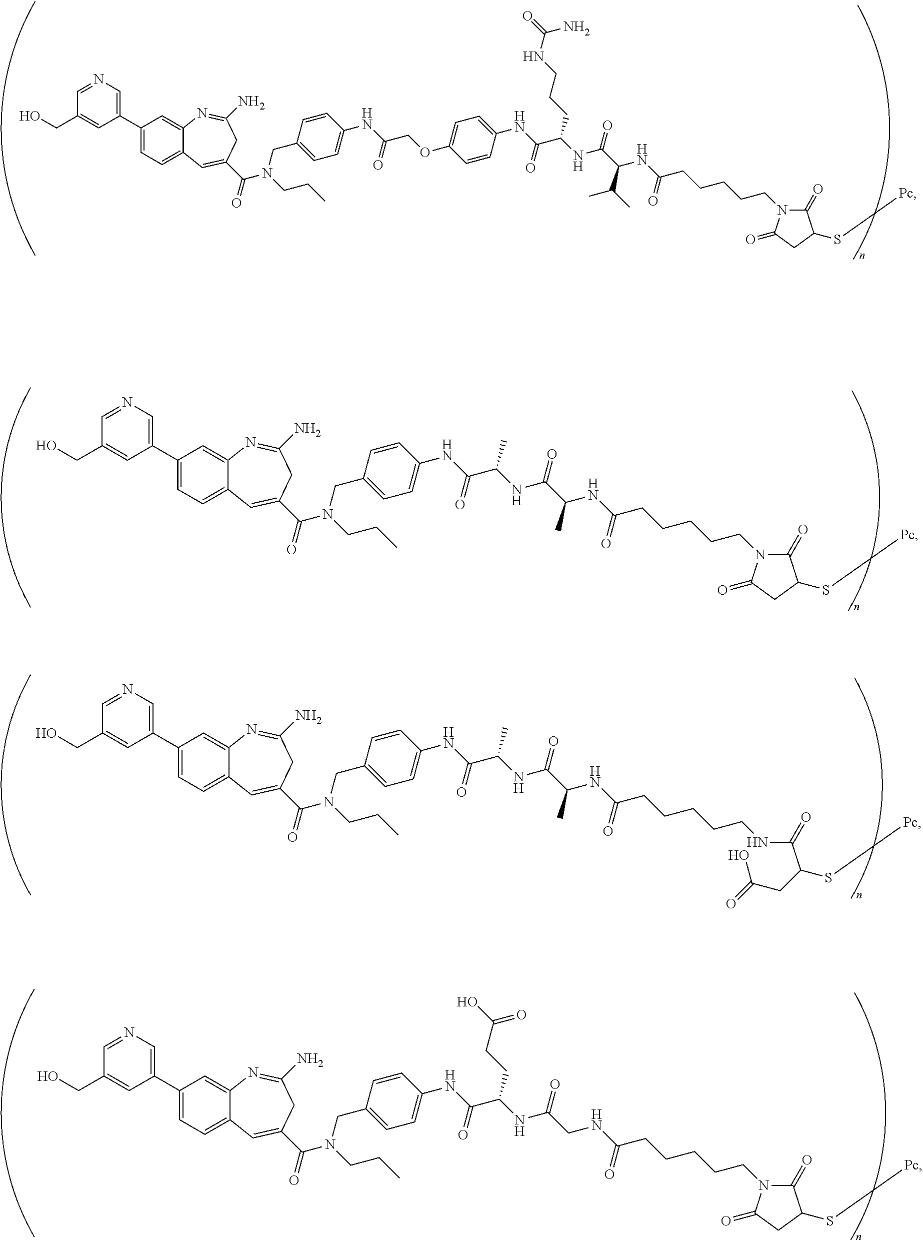

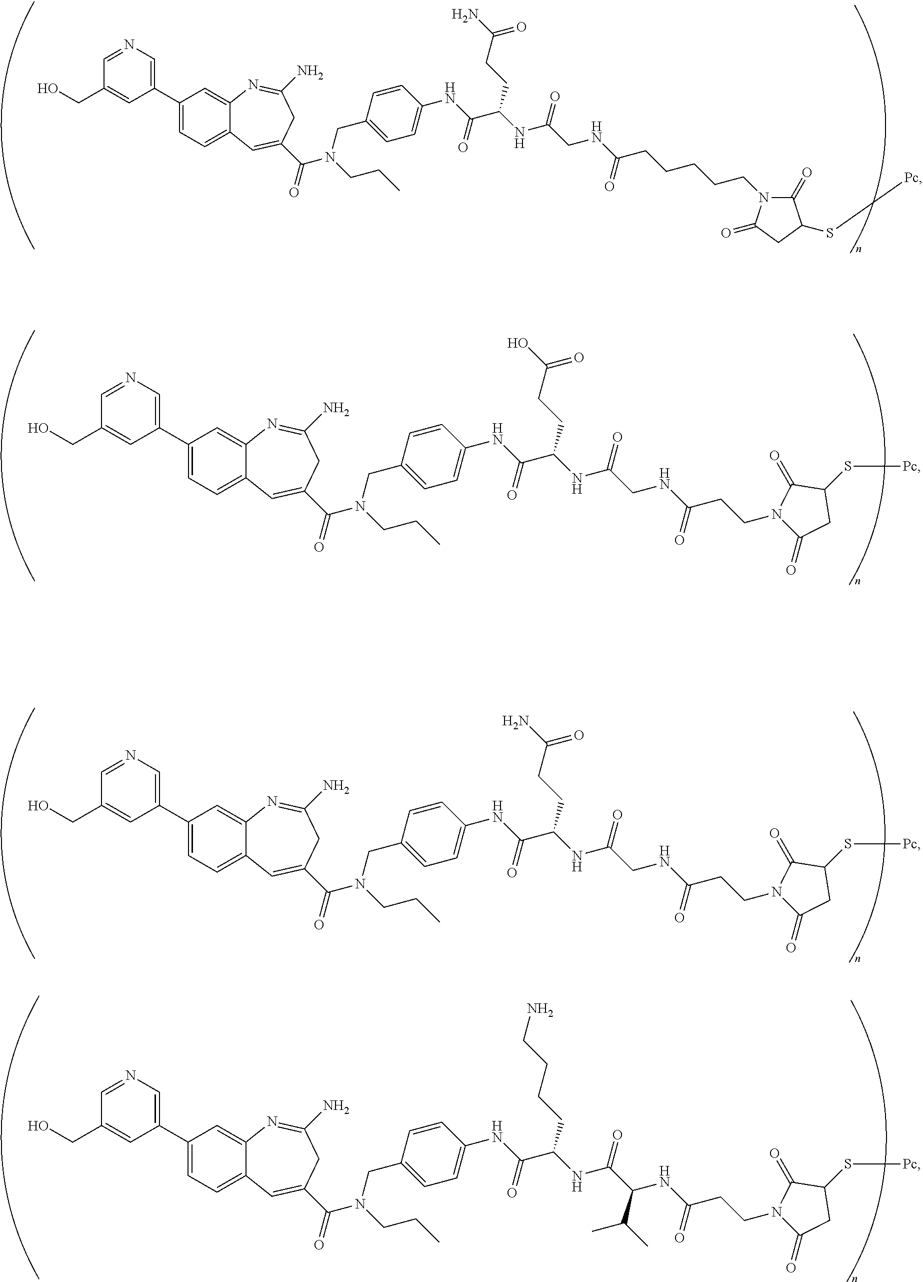

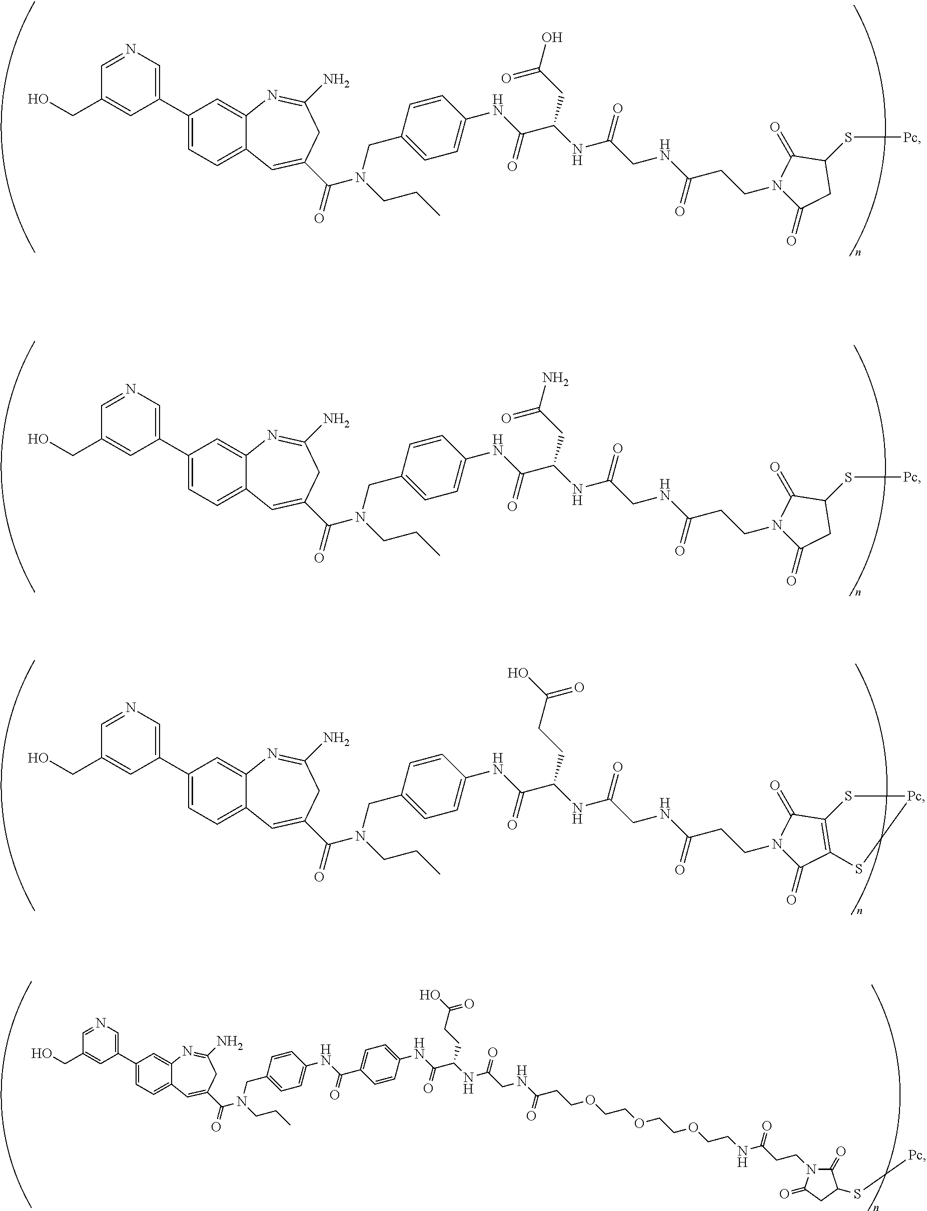

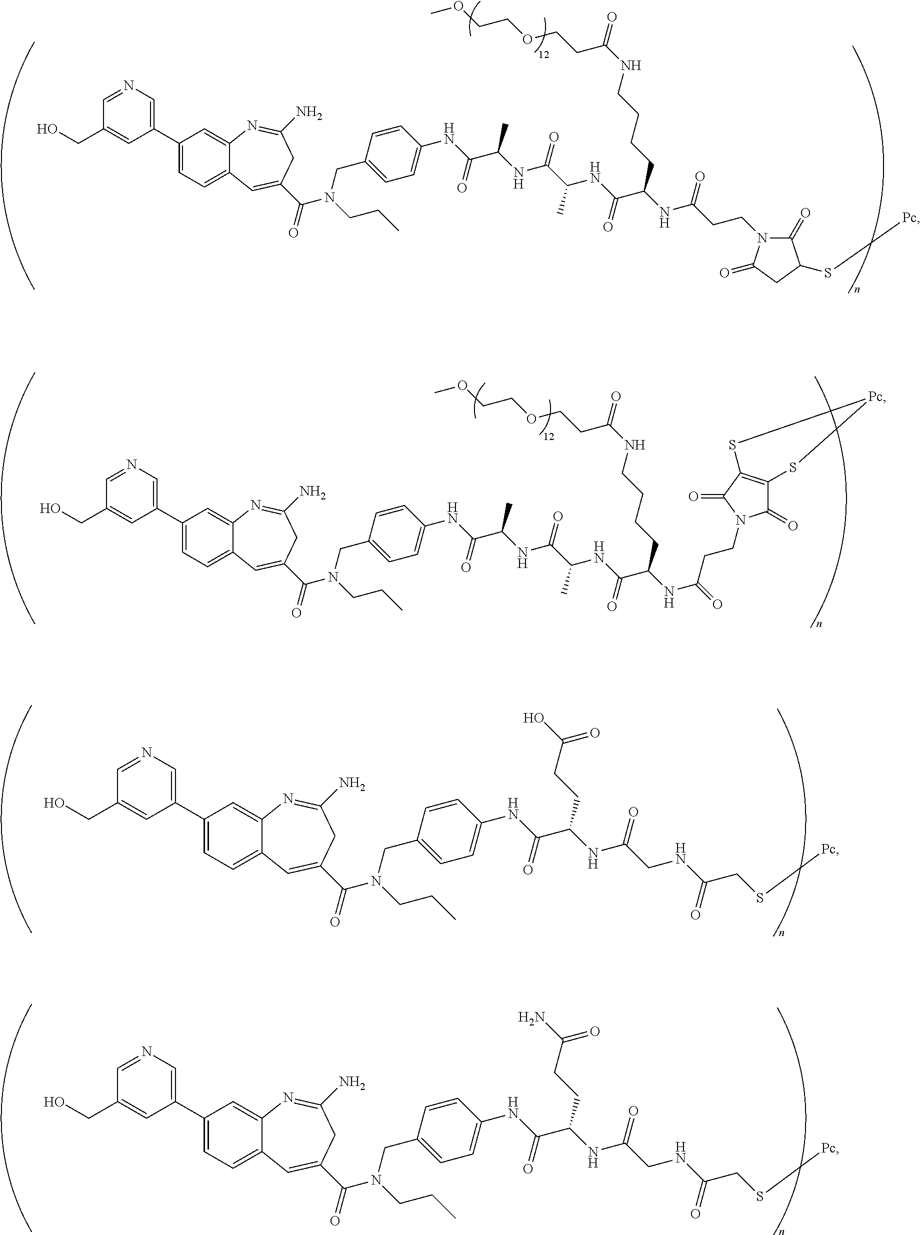

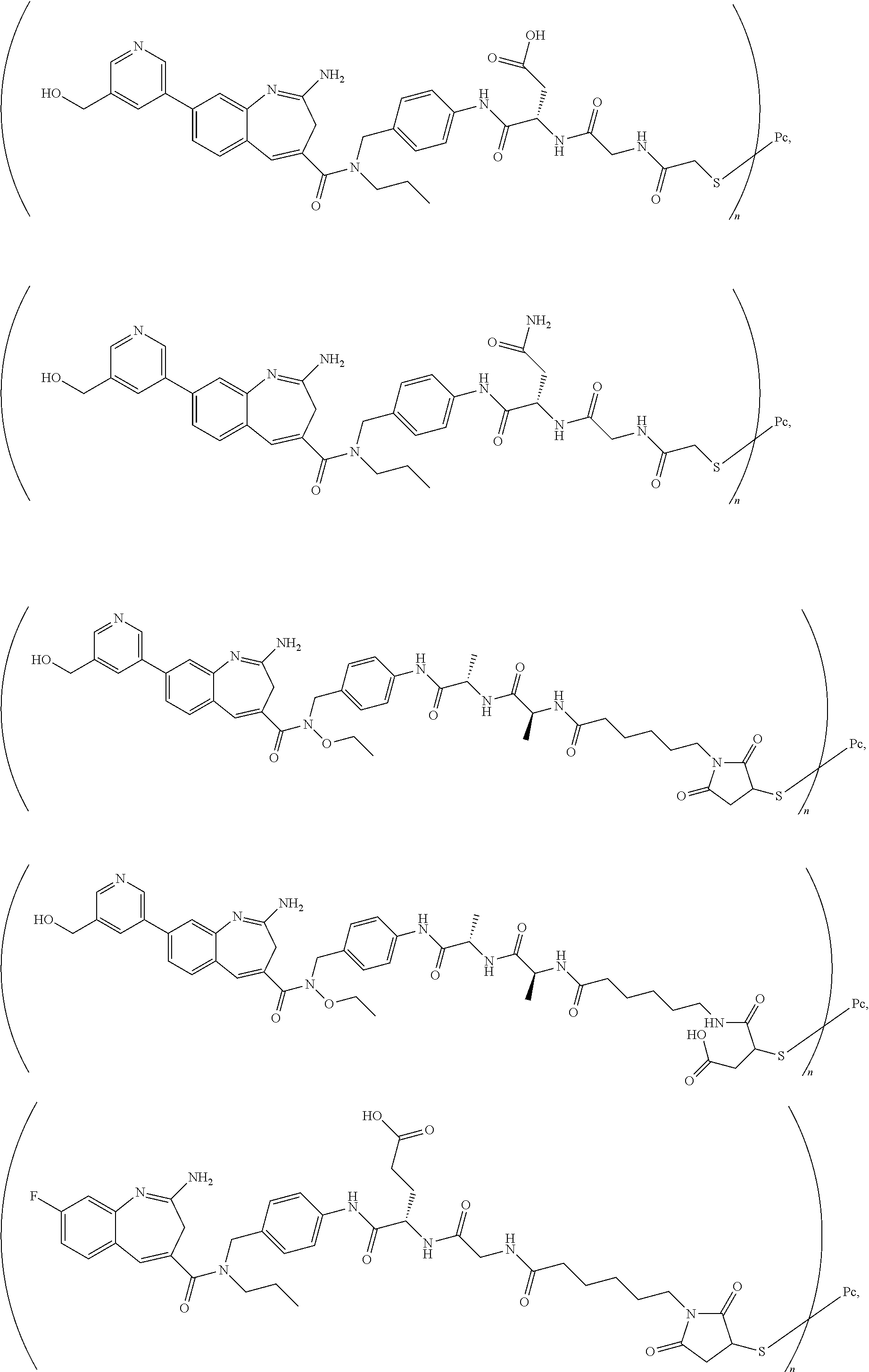

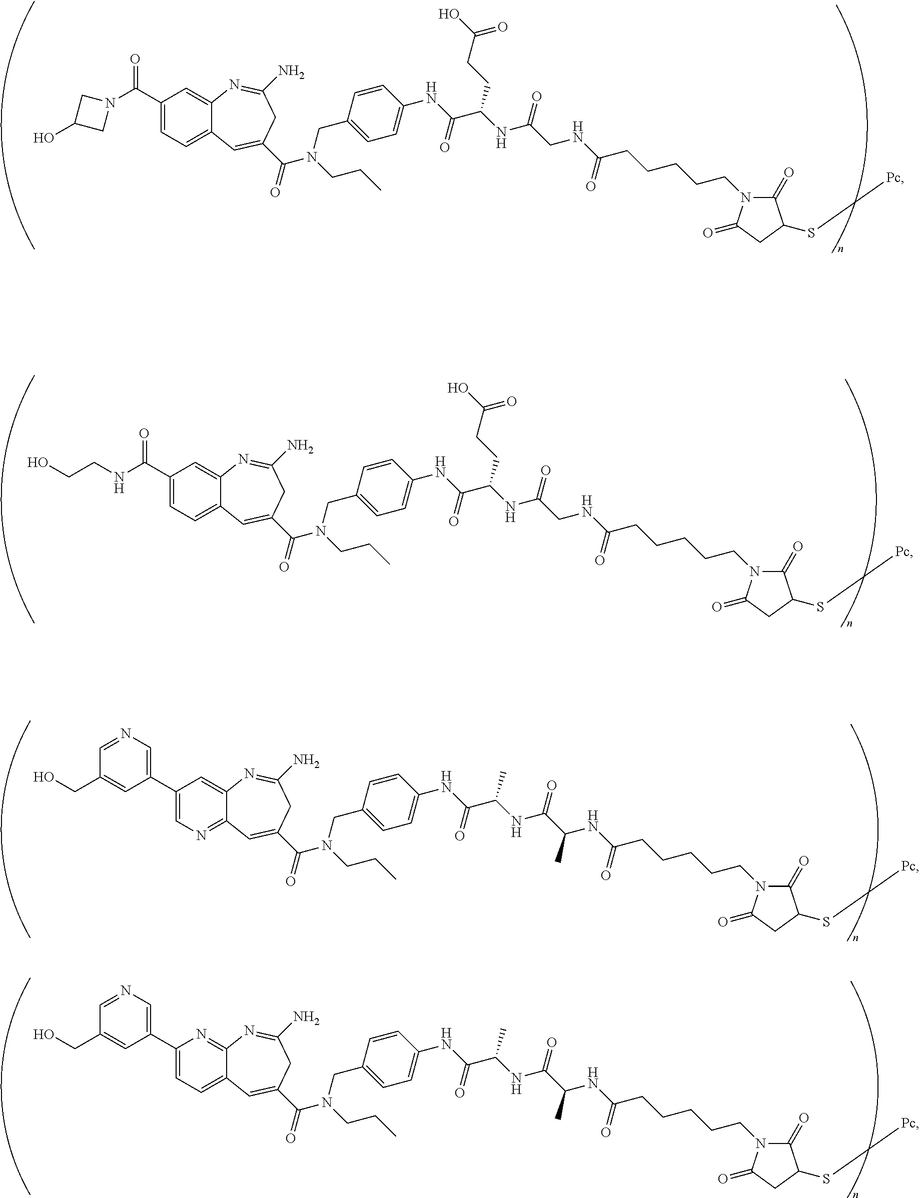

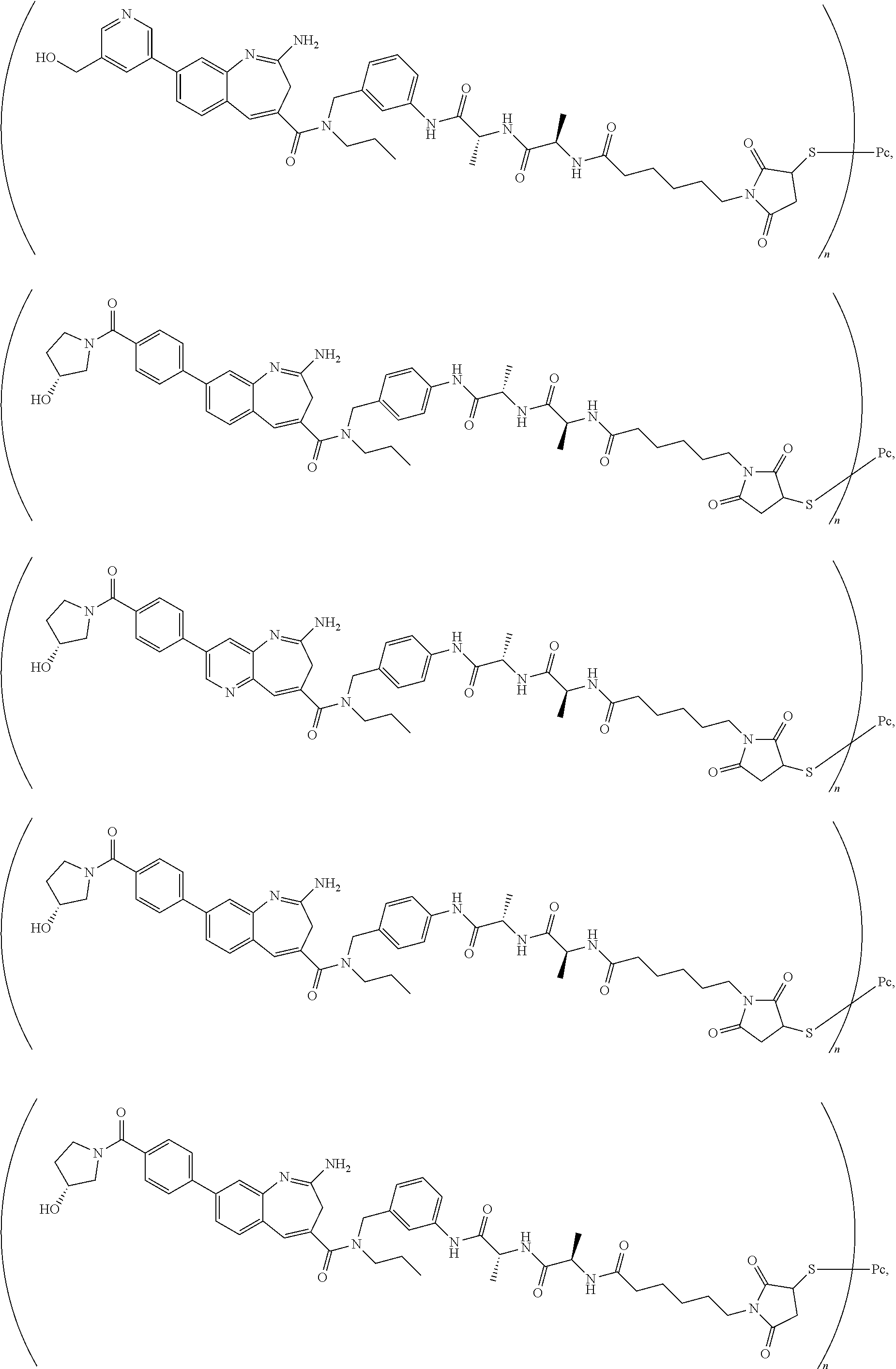

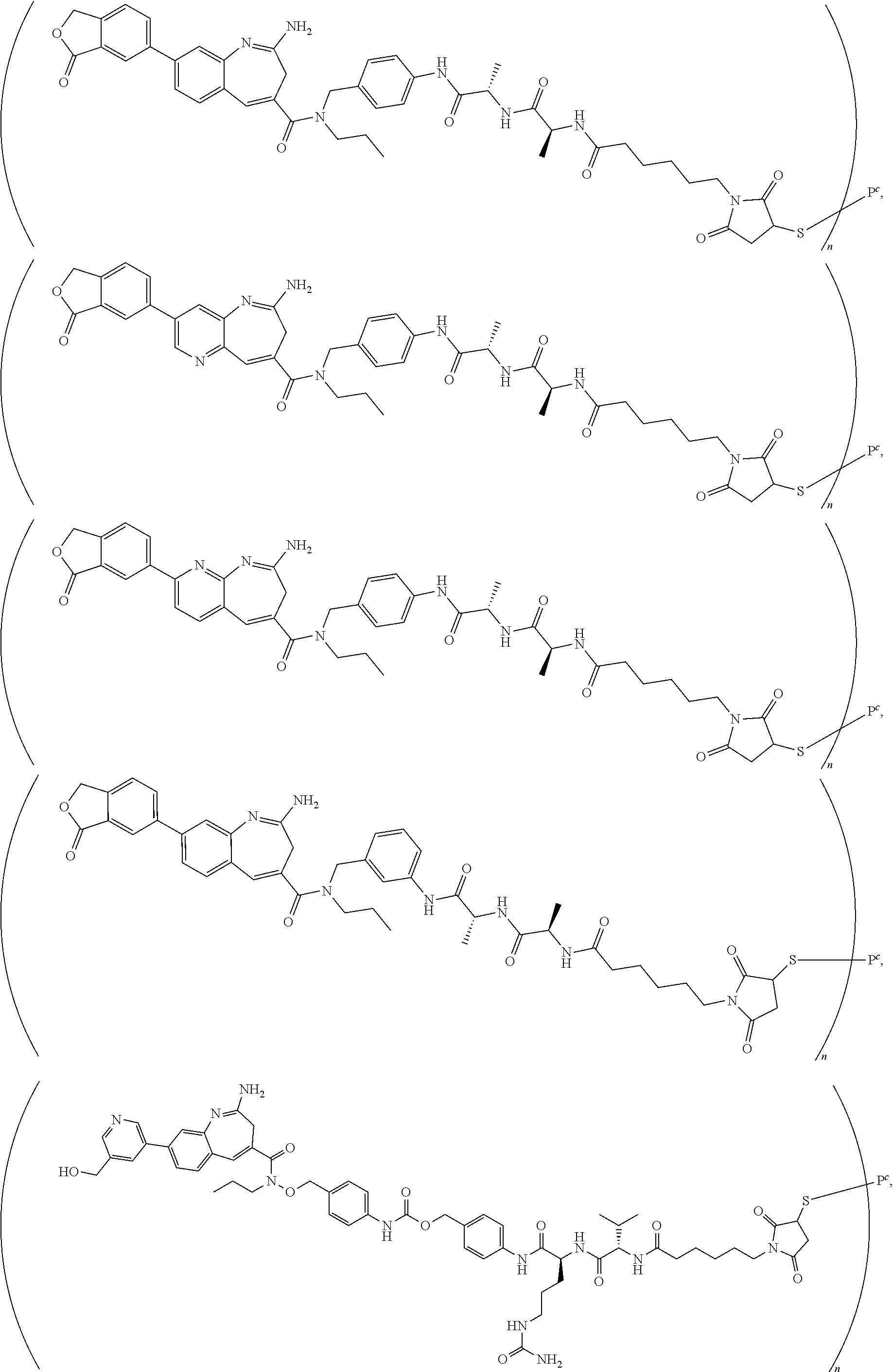

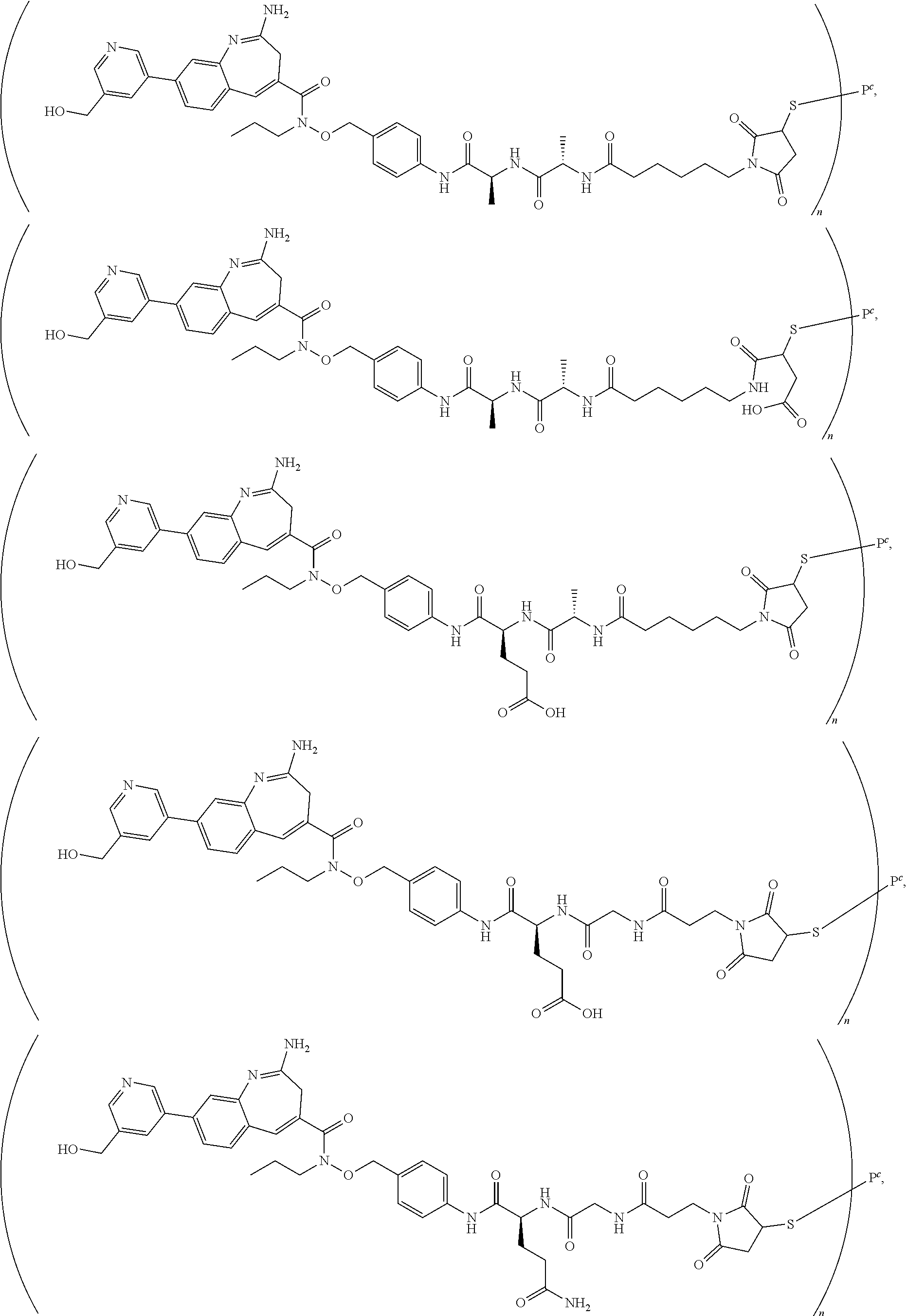

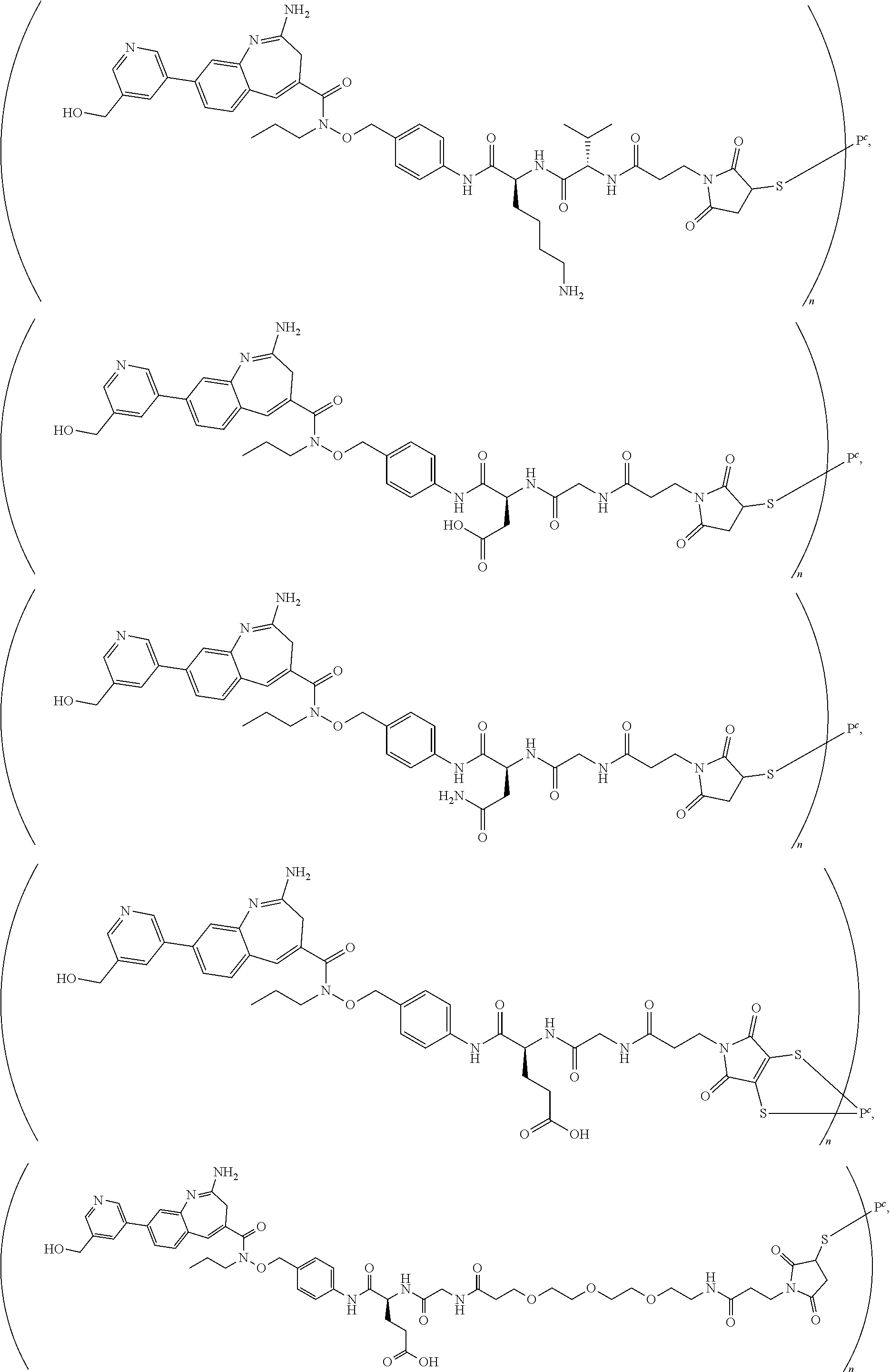

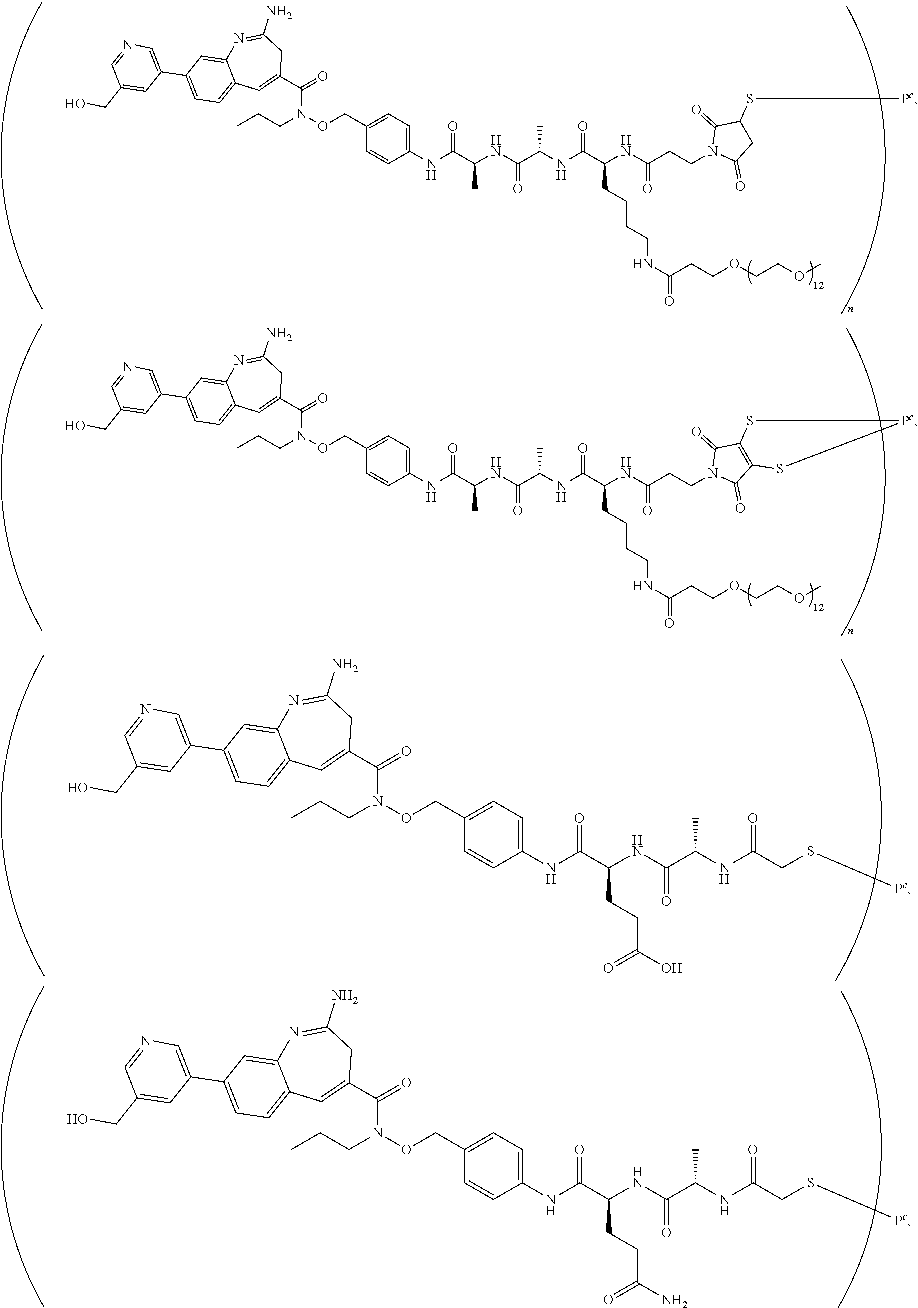

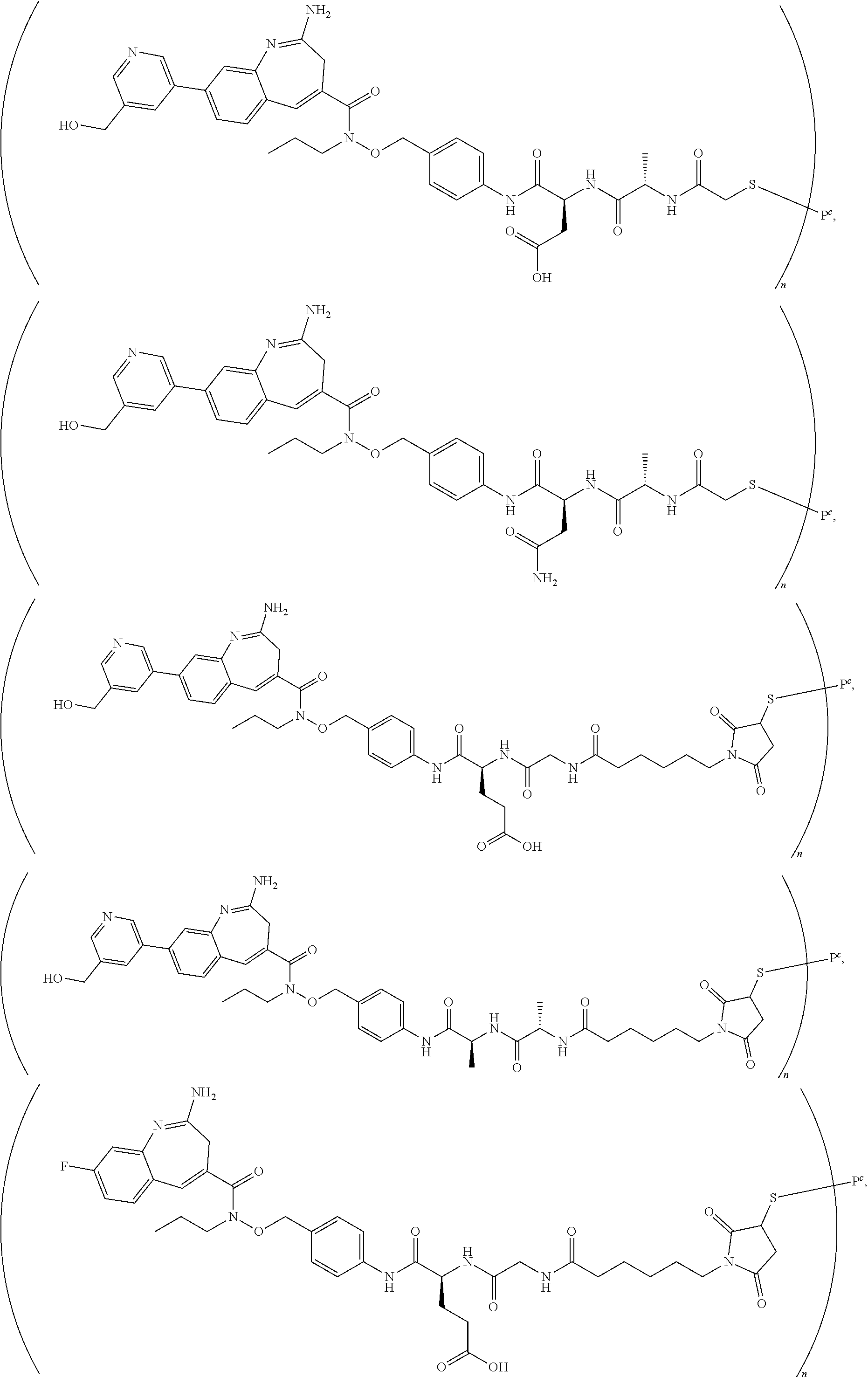

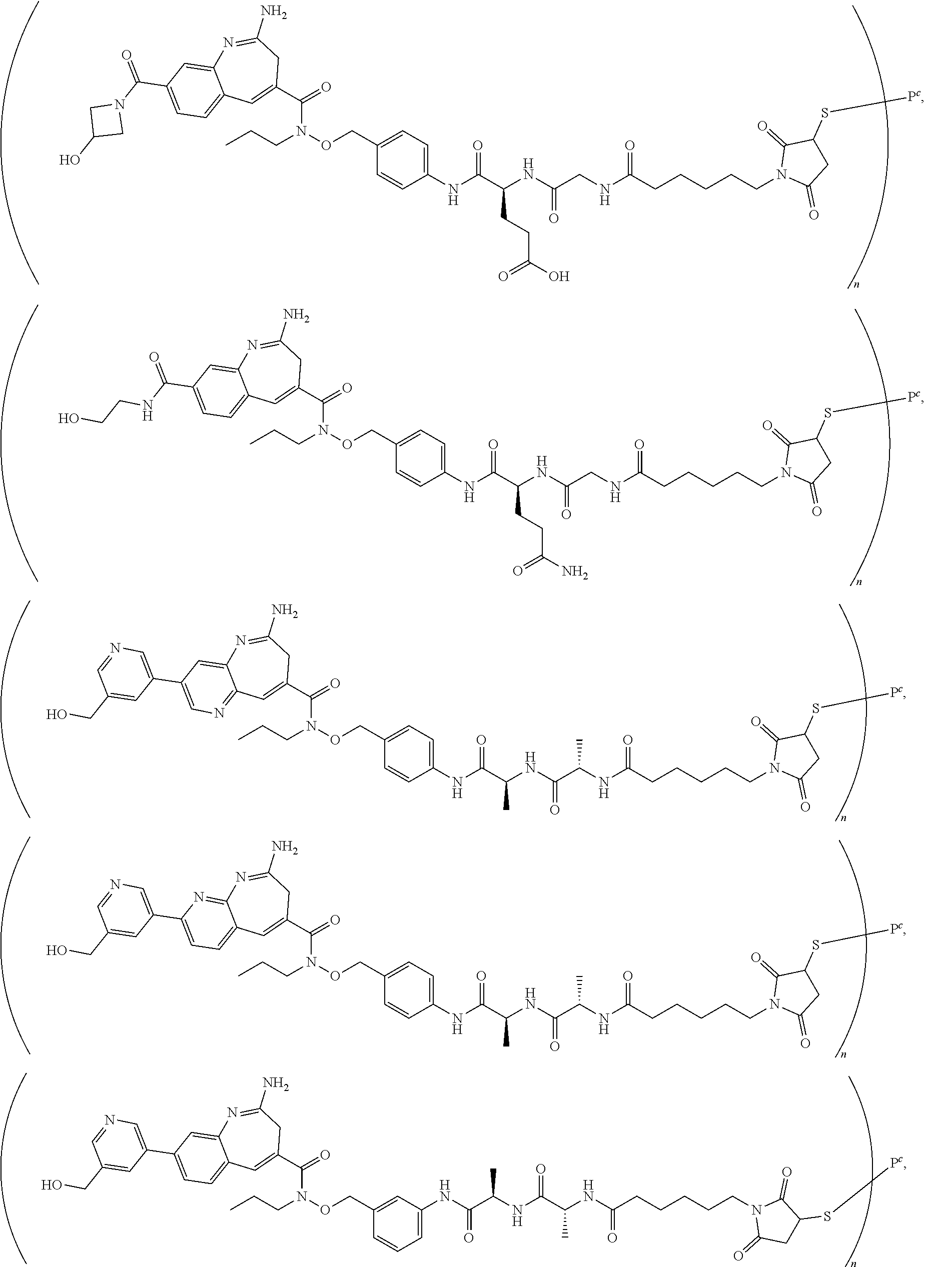

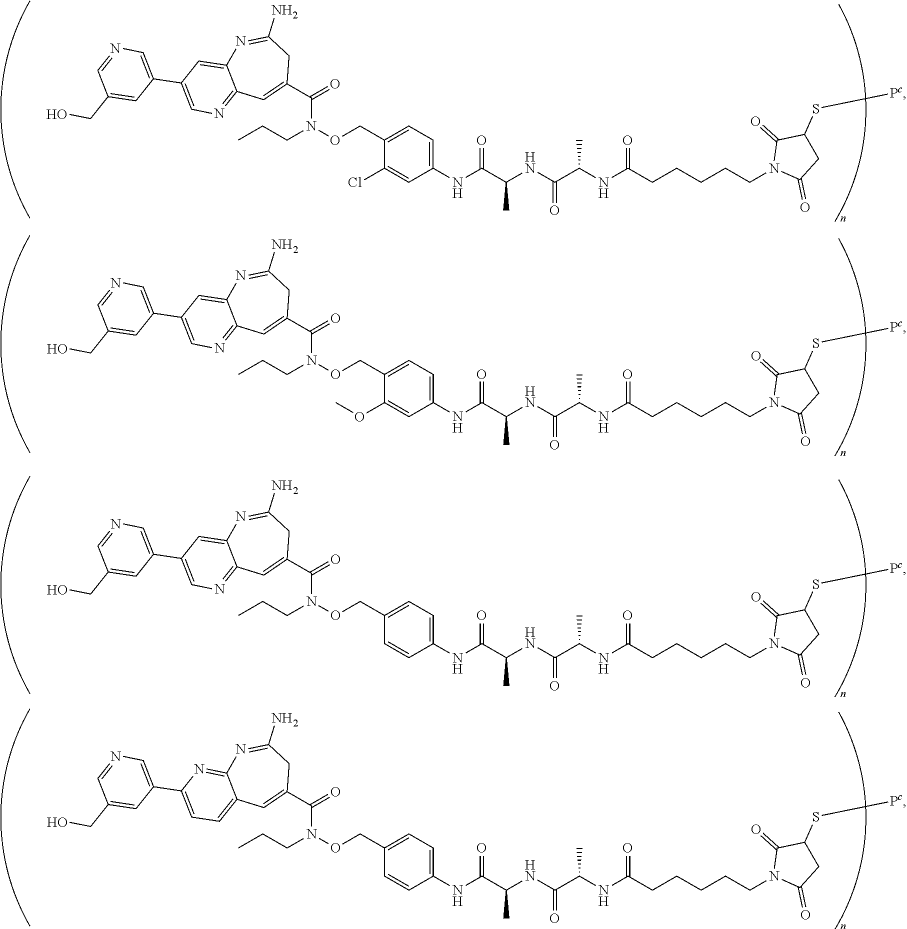

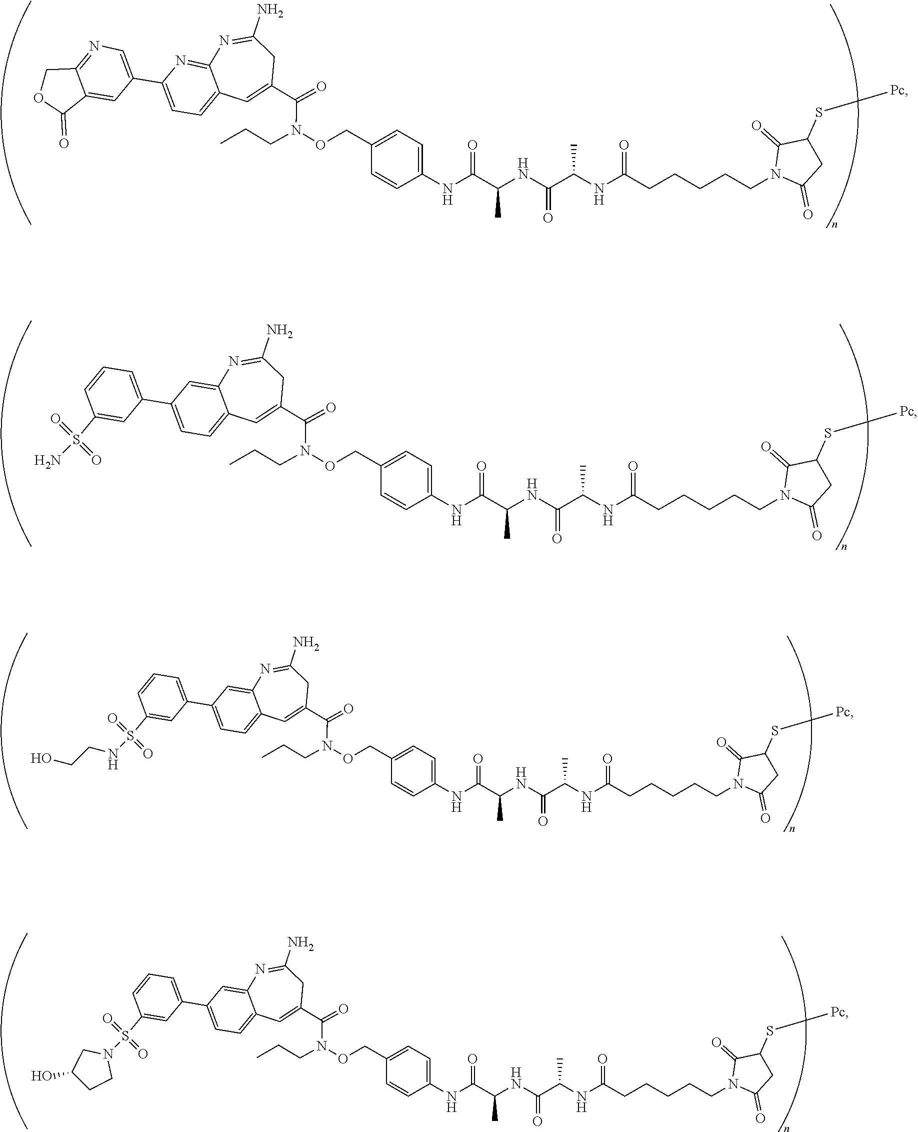

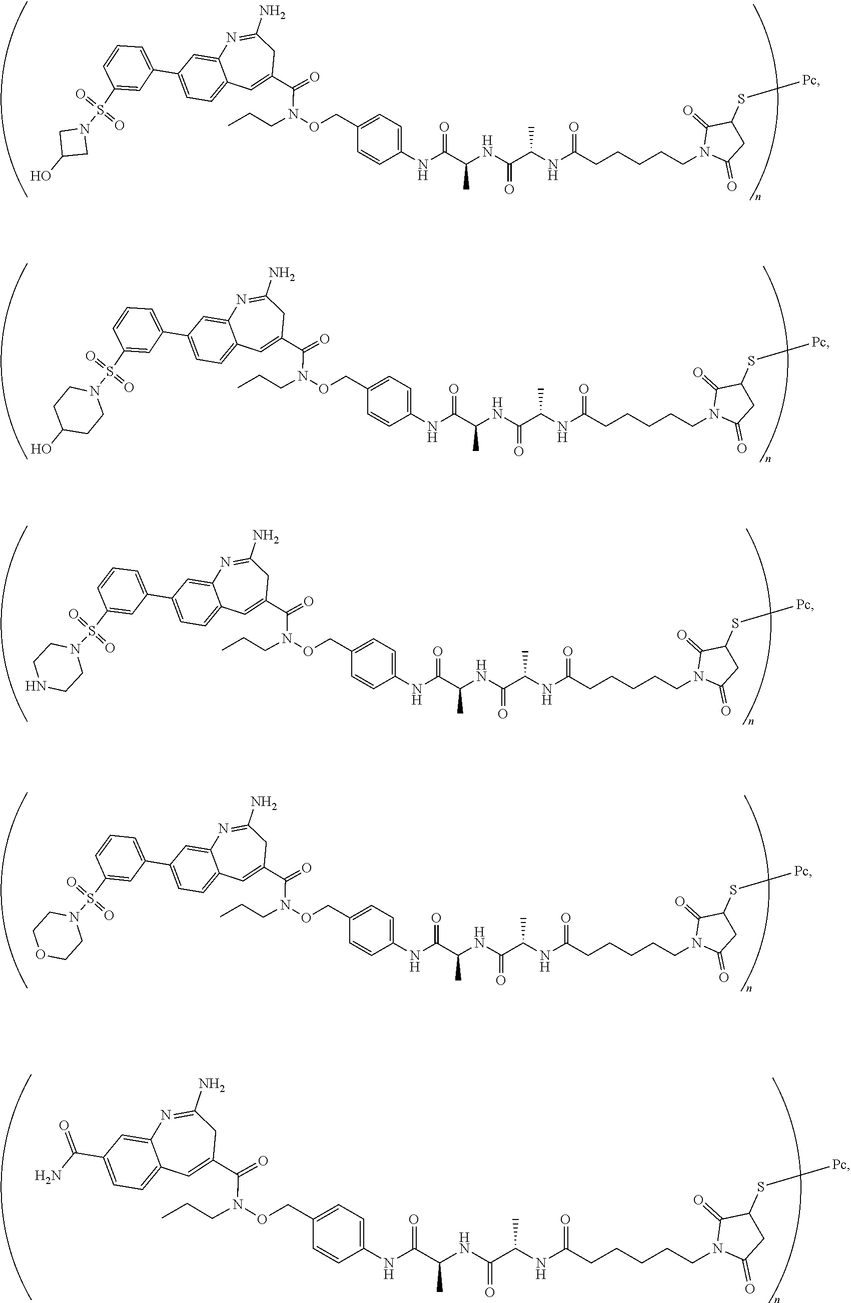

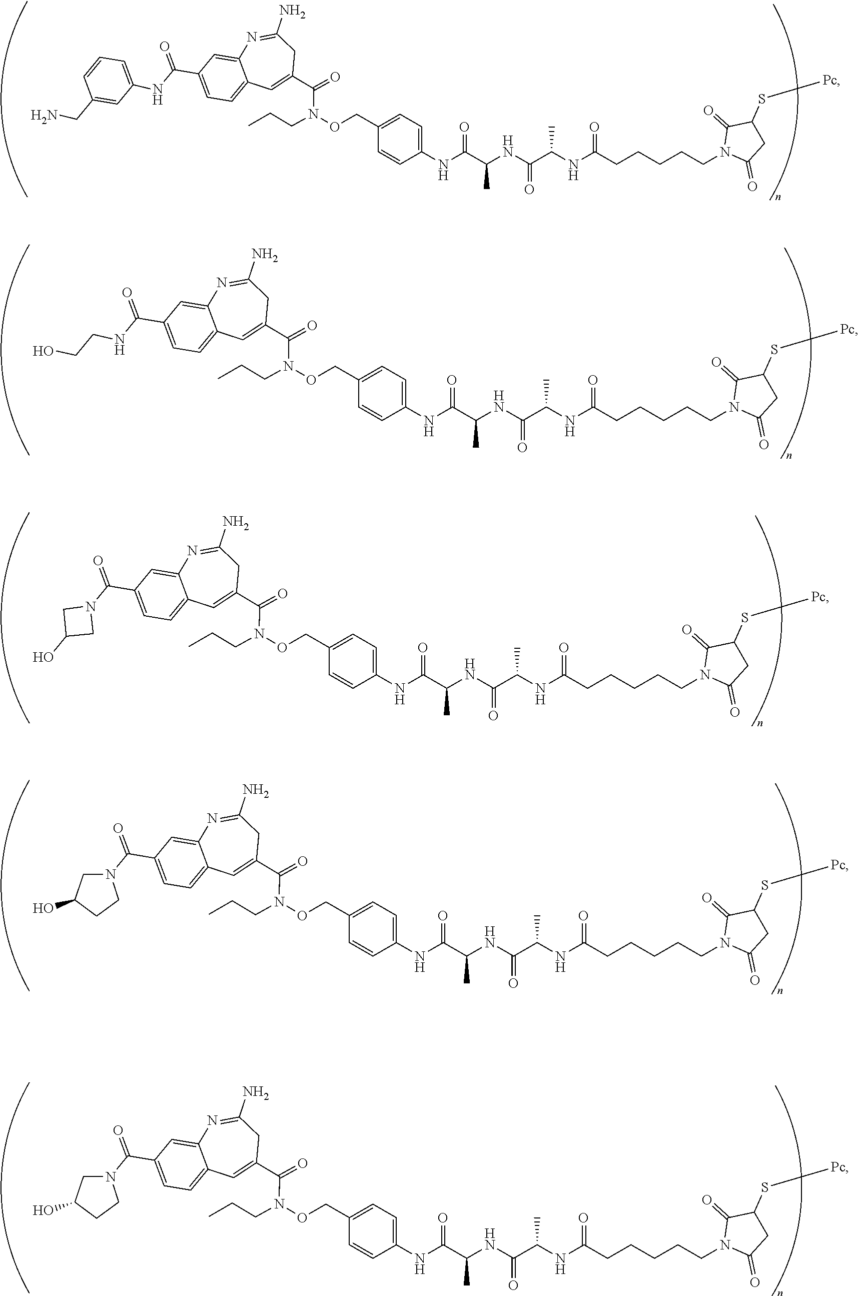

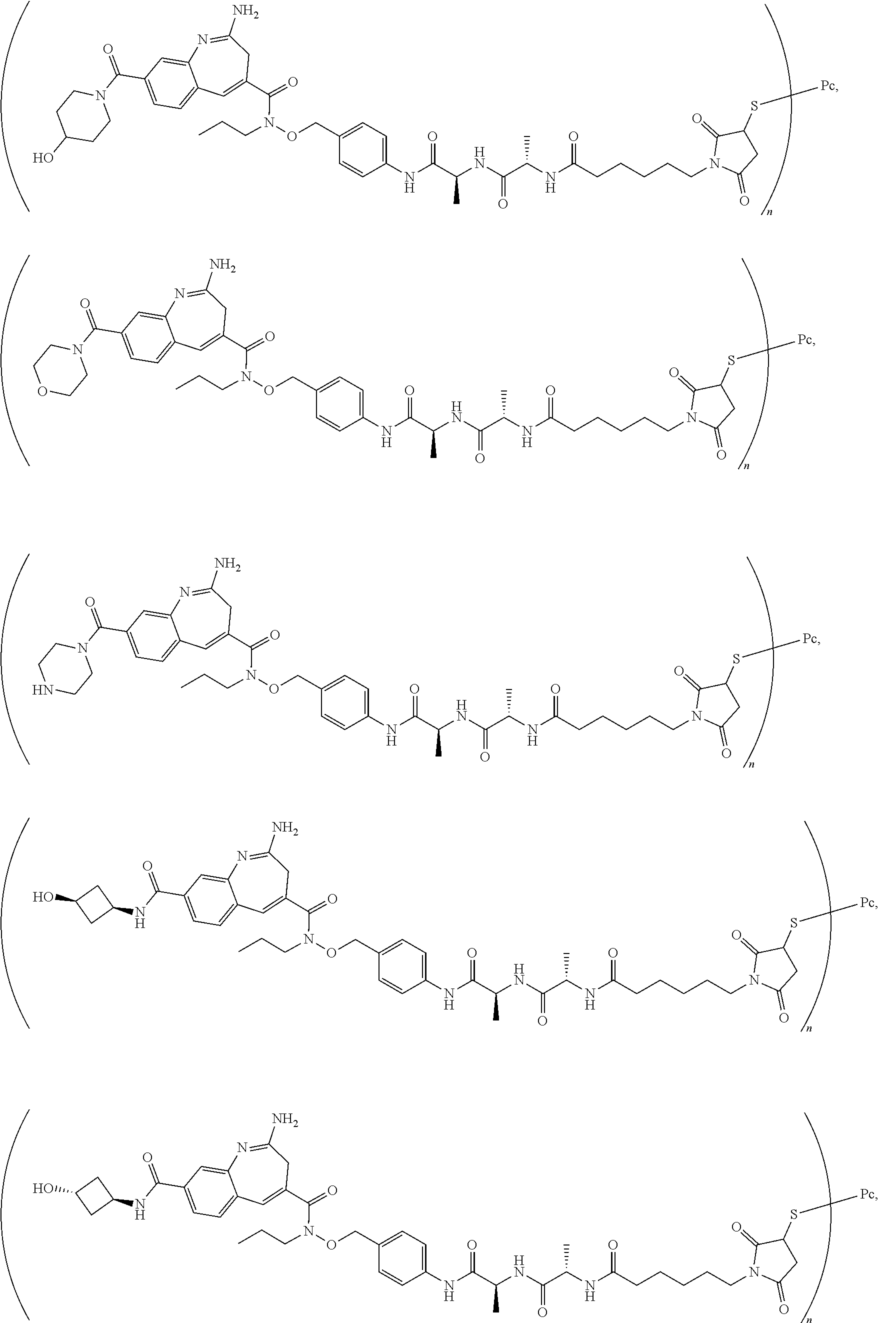

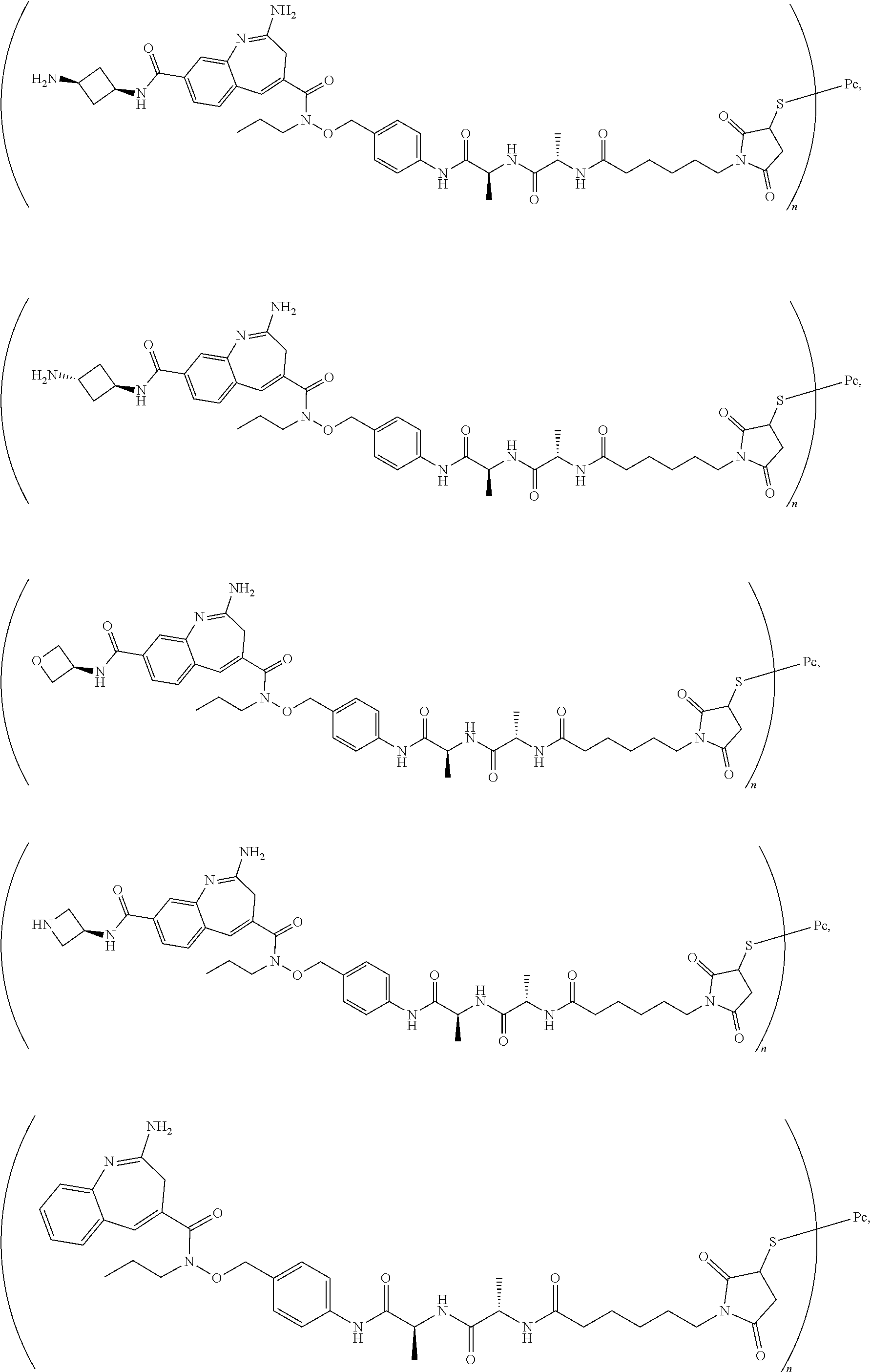

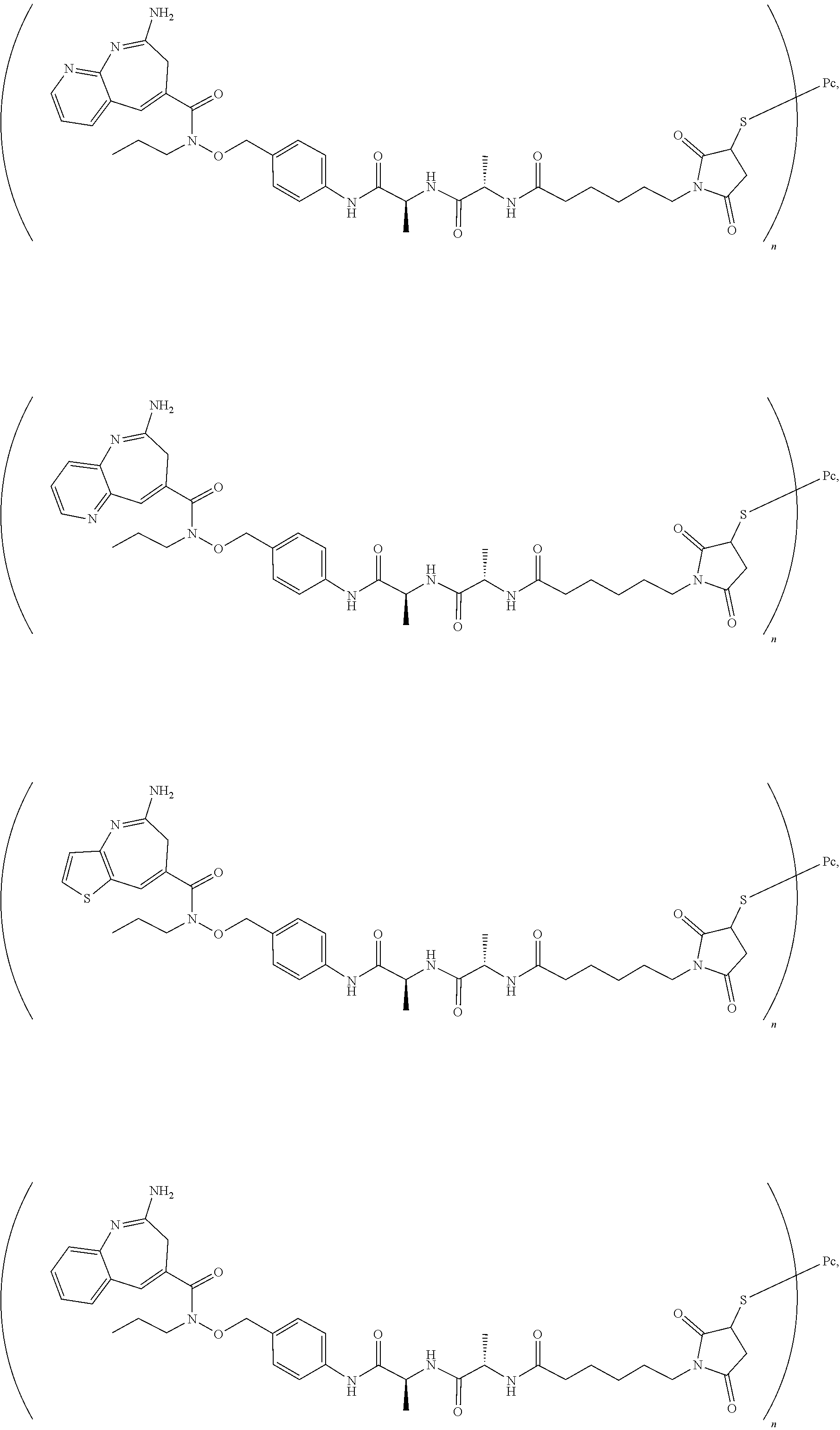

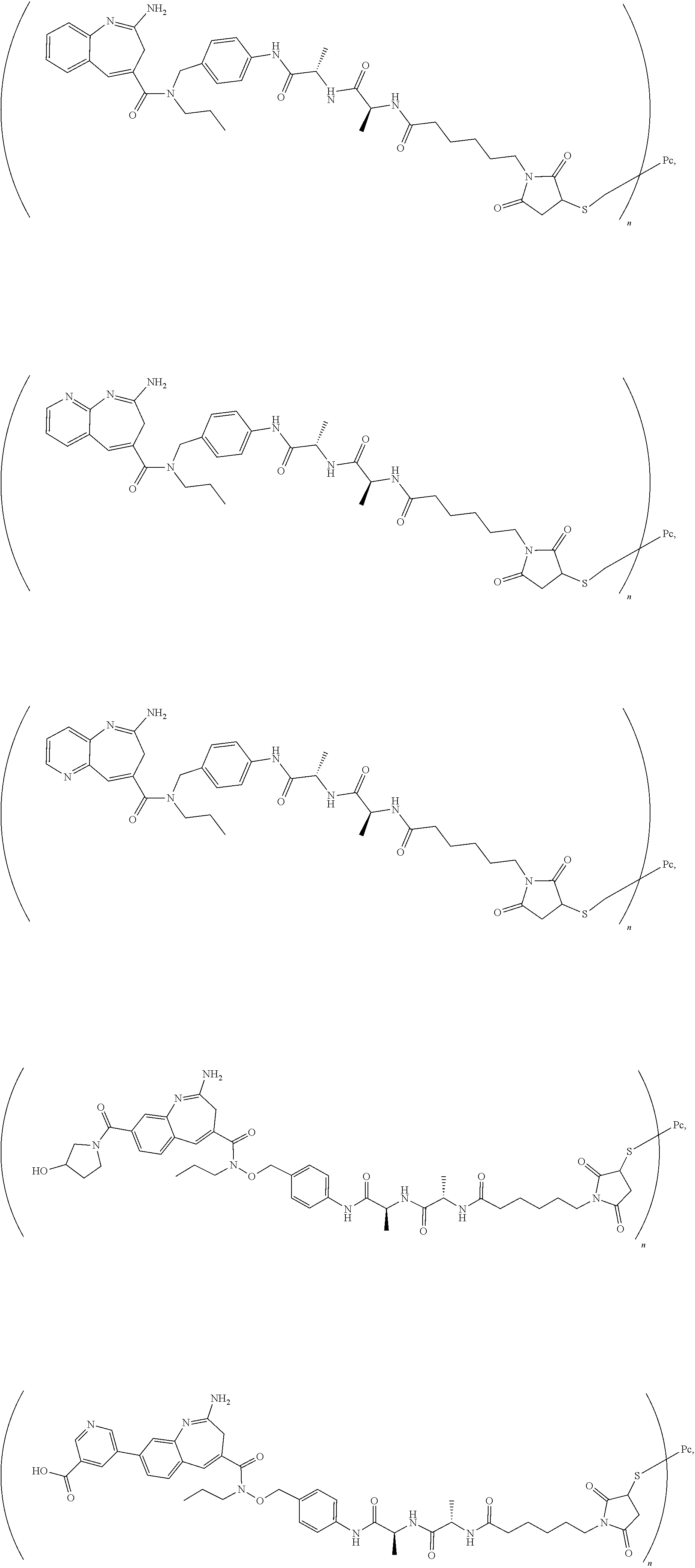

n is an integer or a decimal from 1 to 10;
Pc is a ligand.

In some embodiments, in the ligand-drug conjugate, or the isomer thereof, or the mixture thereof, or the pharmaceutically acceptable salt thereof described herein, the ligand-drug conjugate is selected from the following structural formulas:

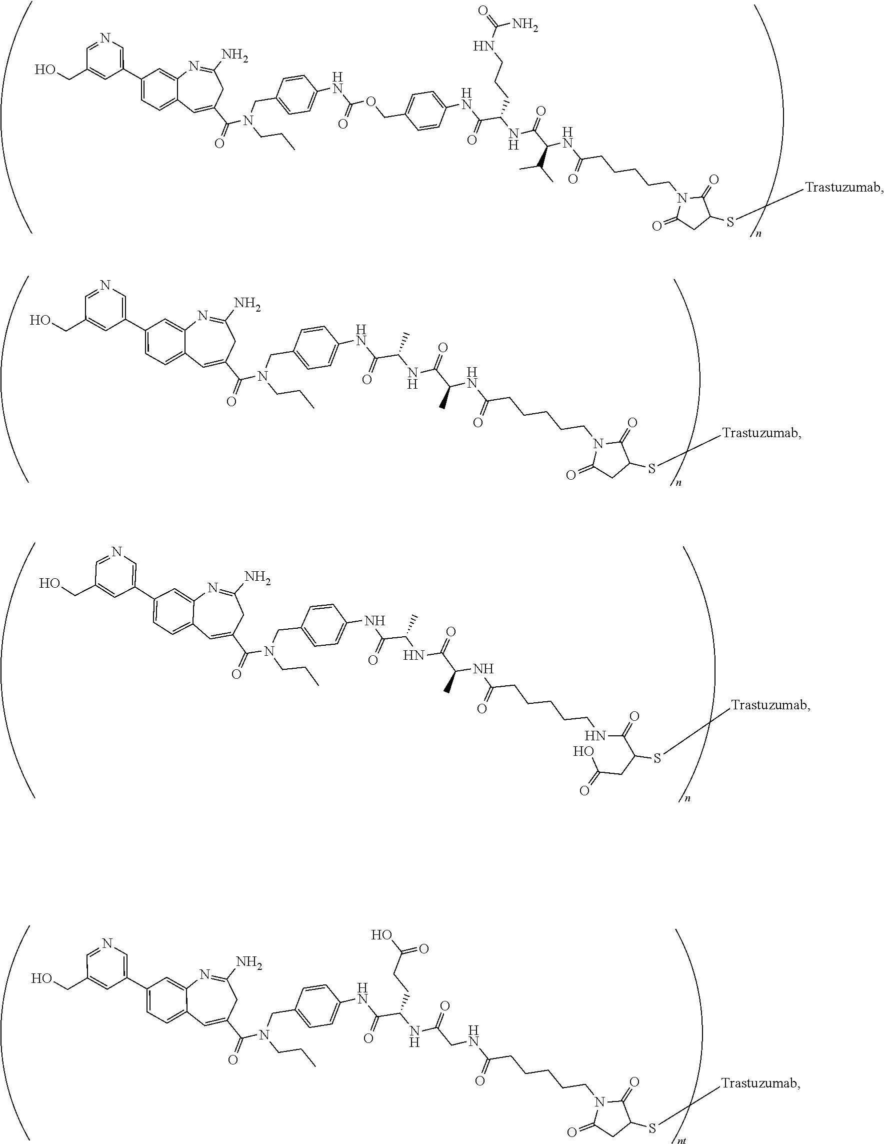

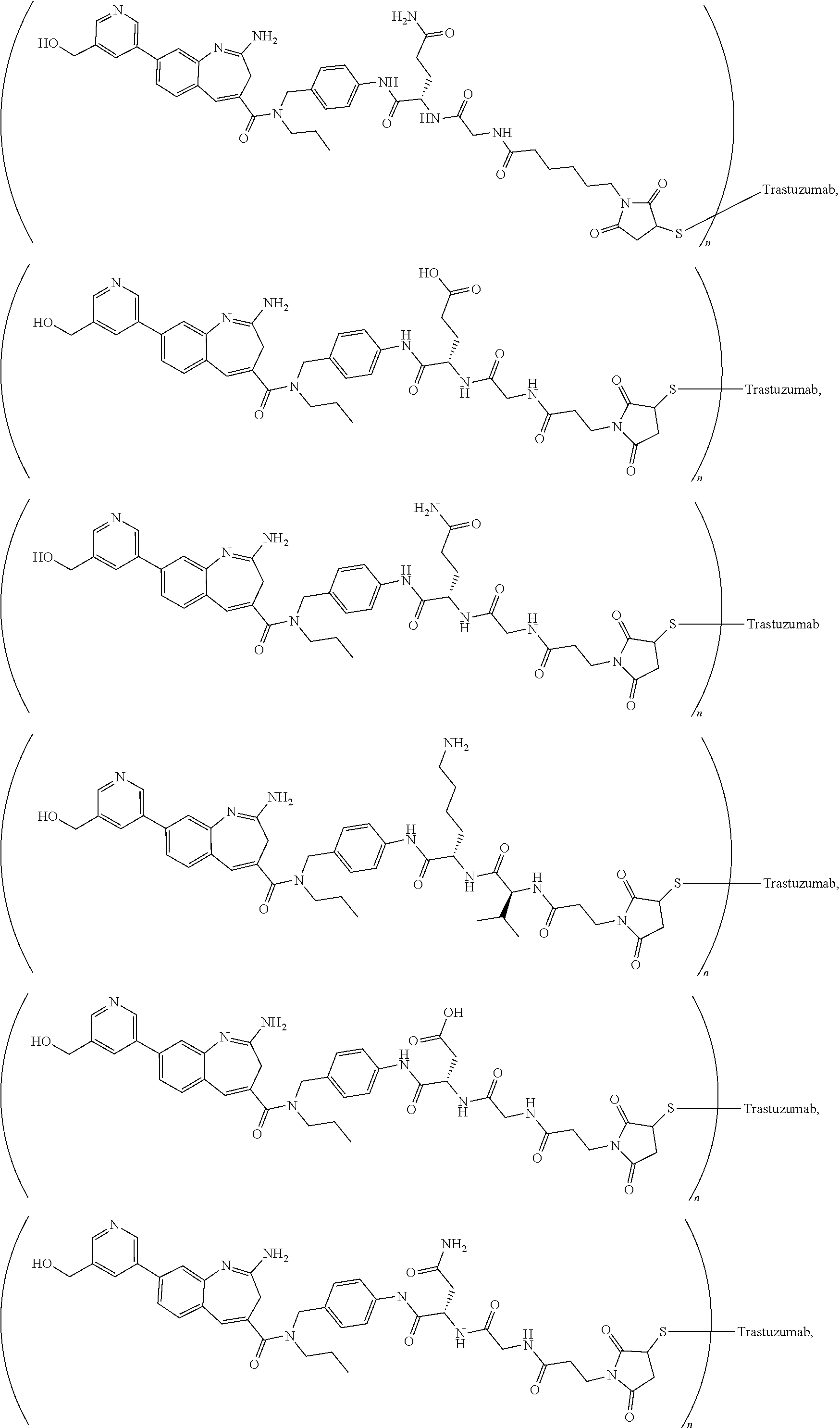

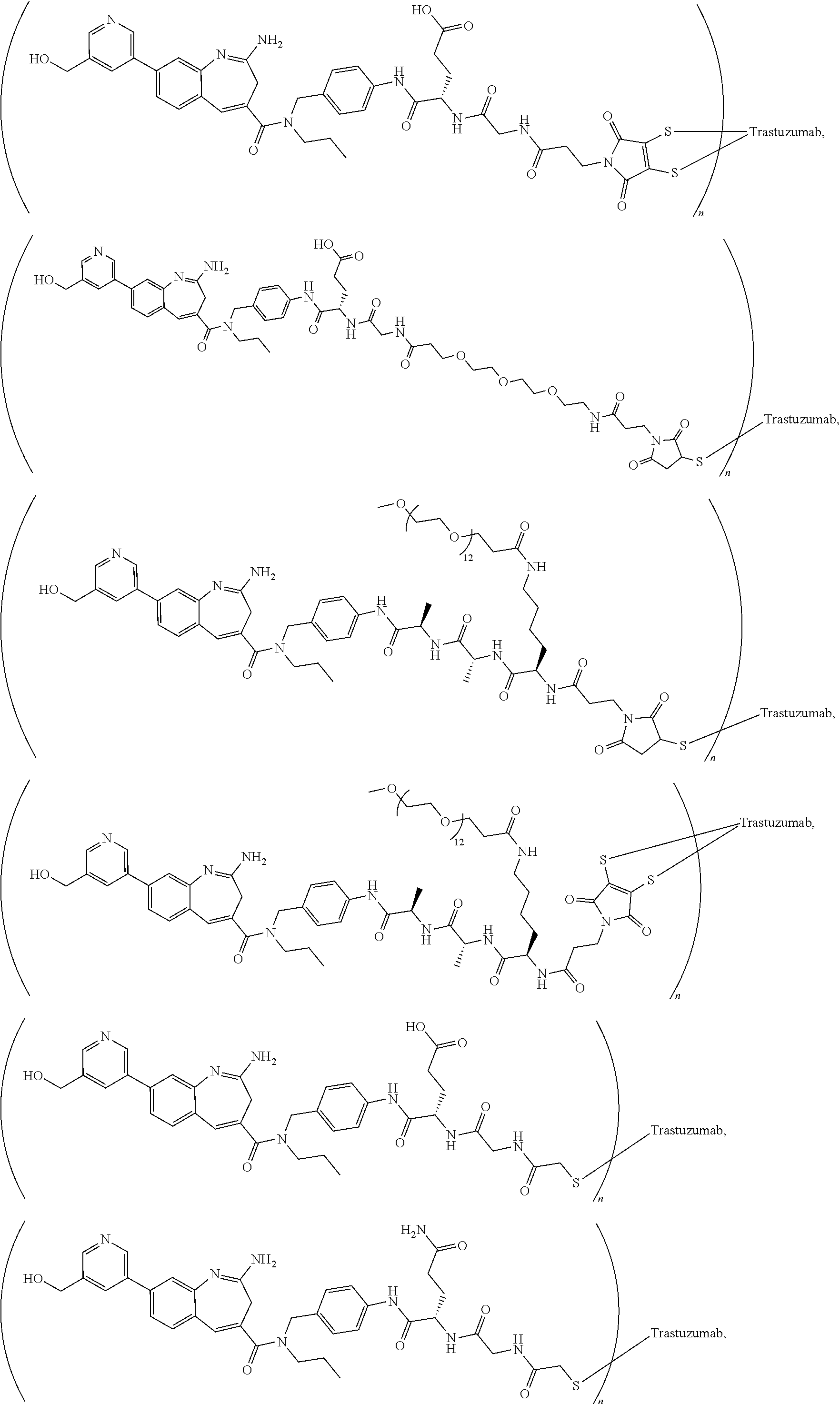

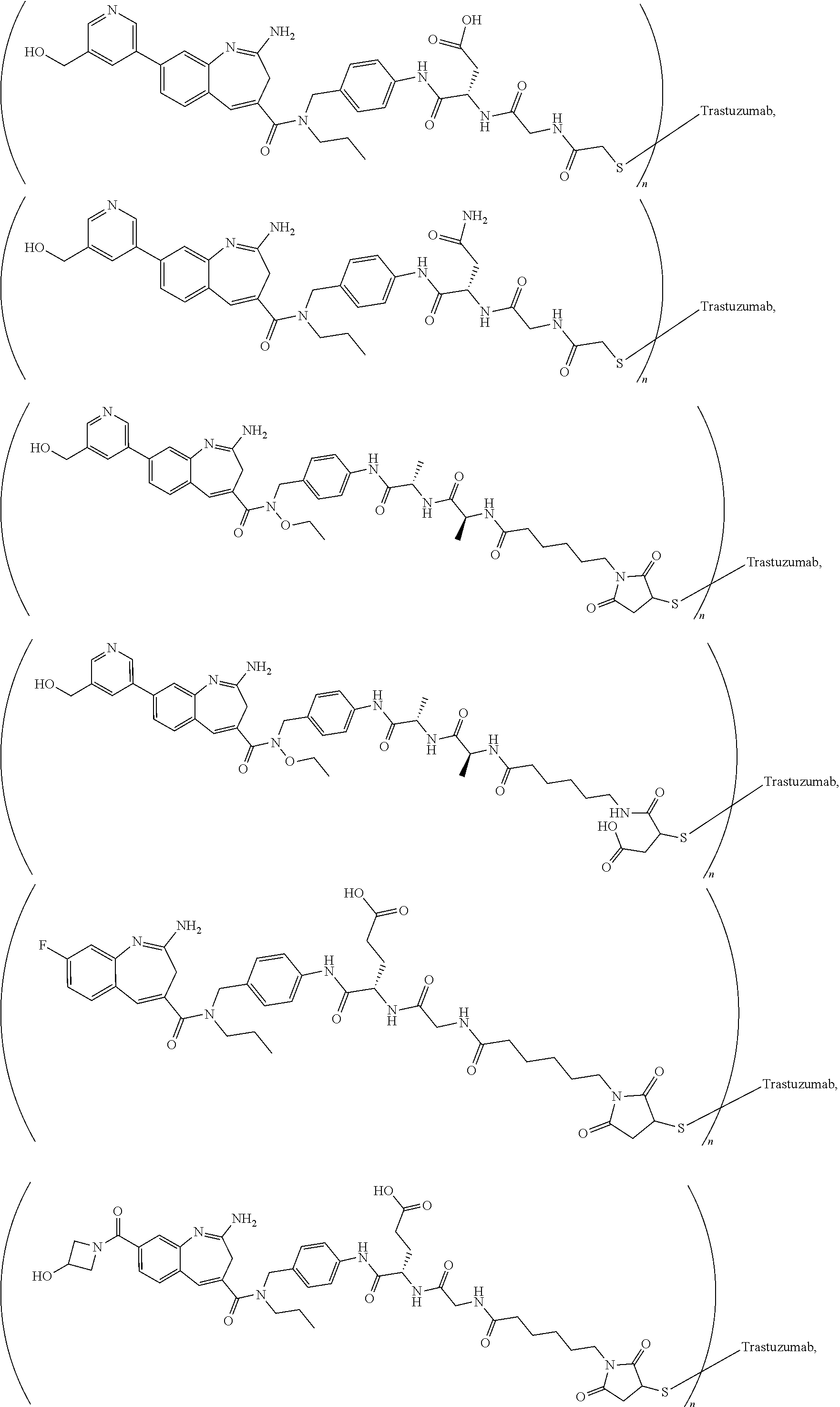

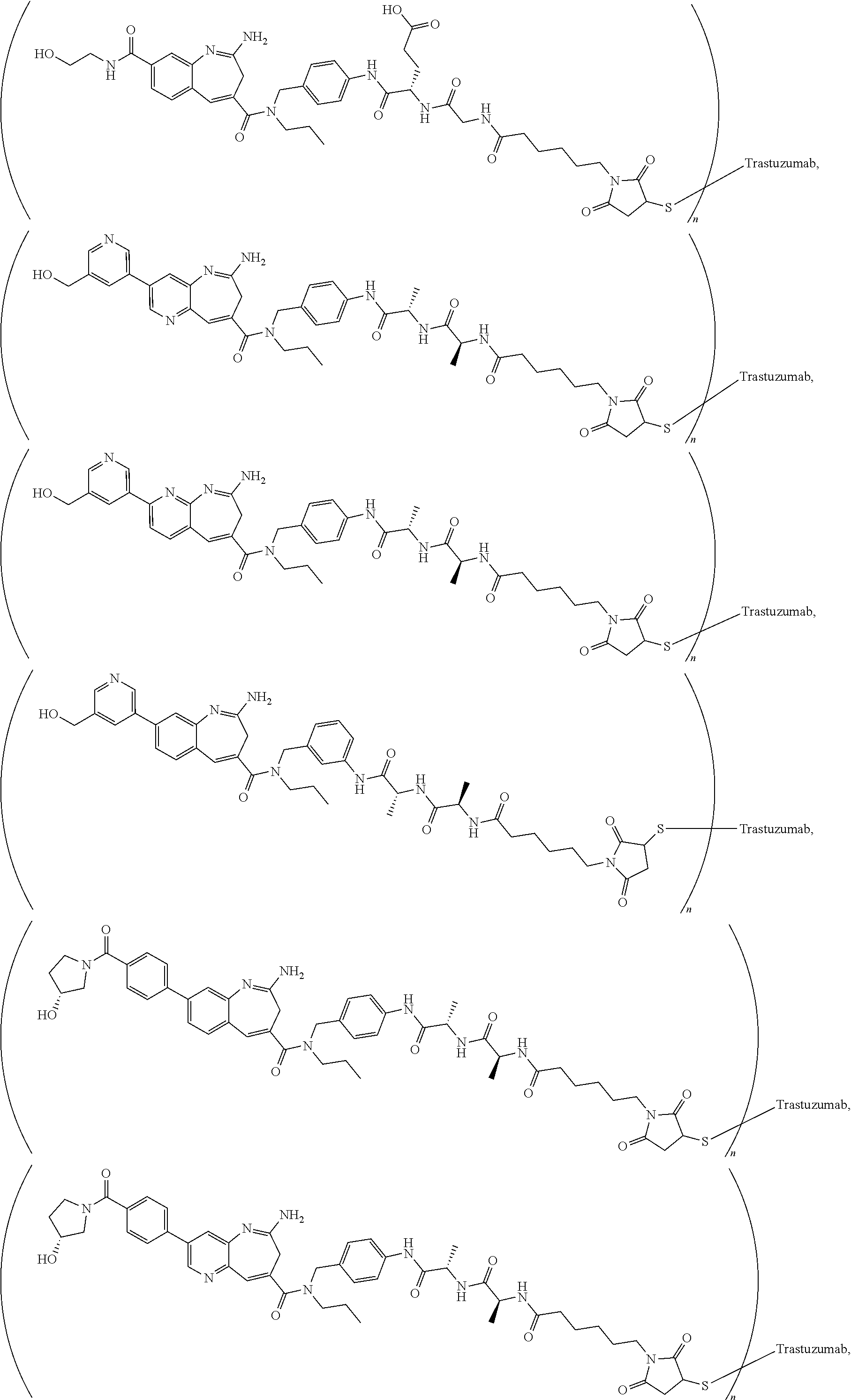

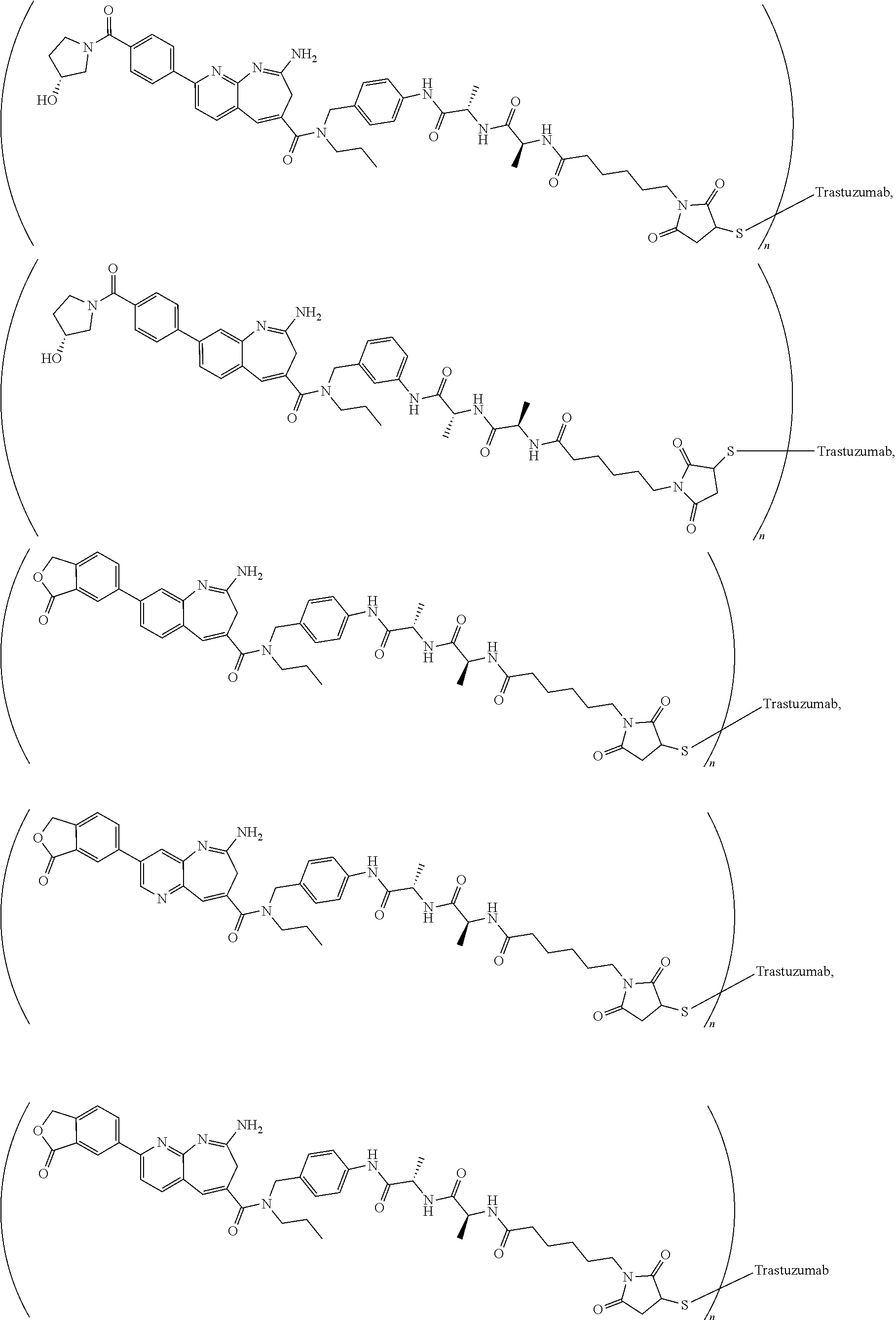

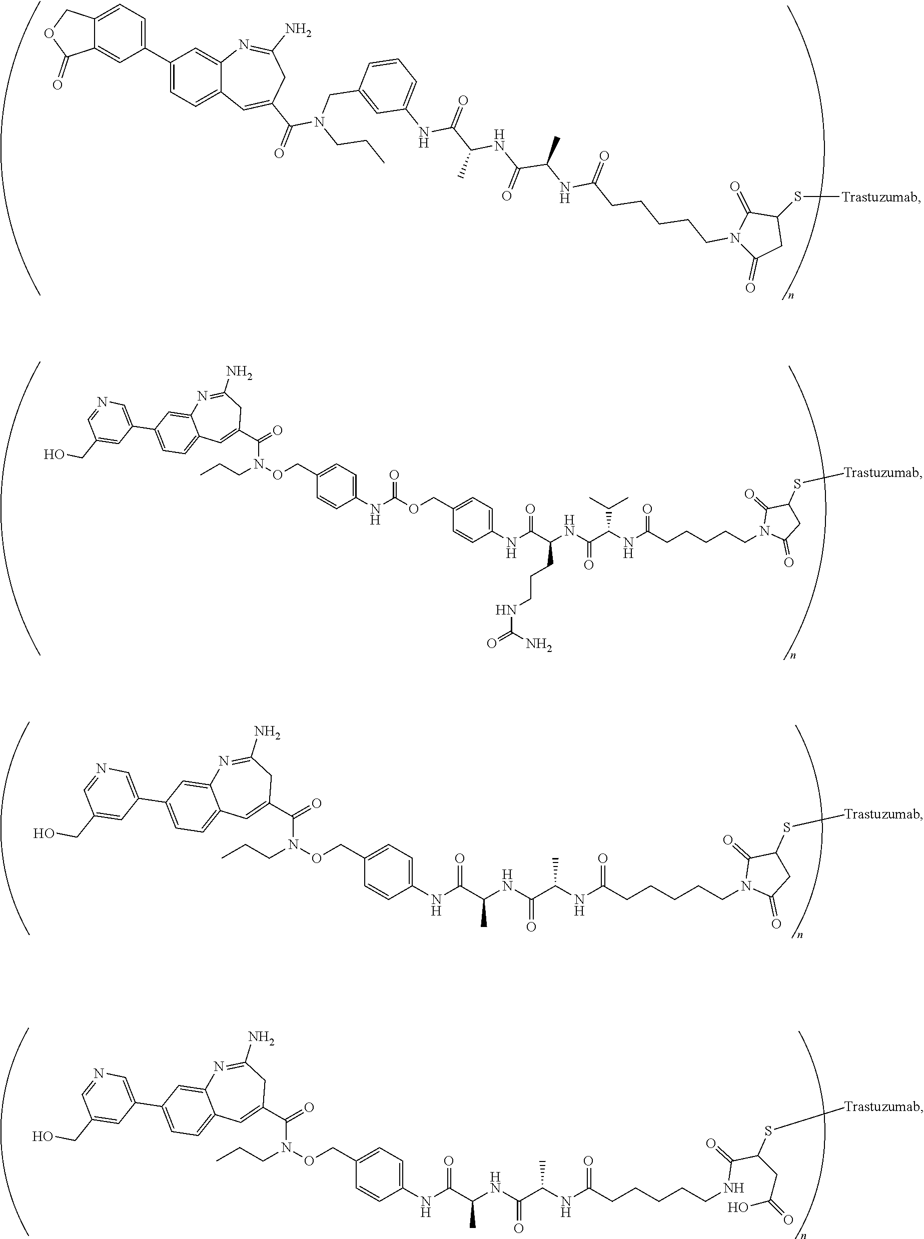

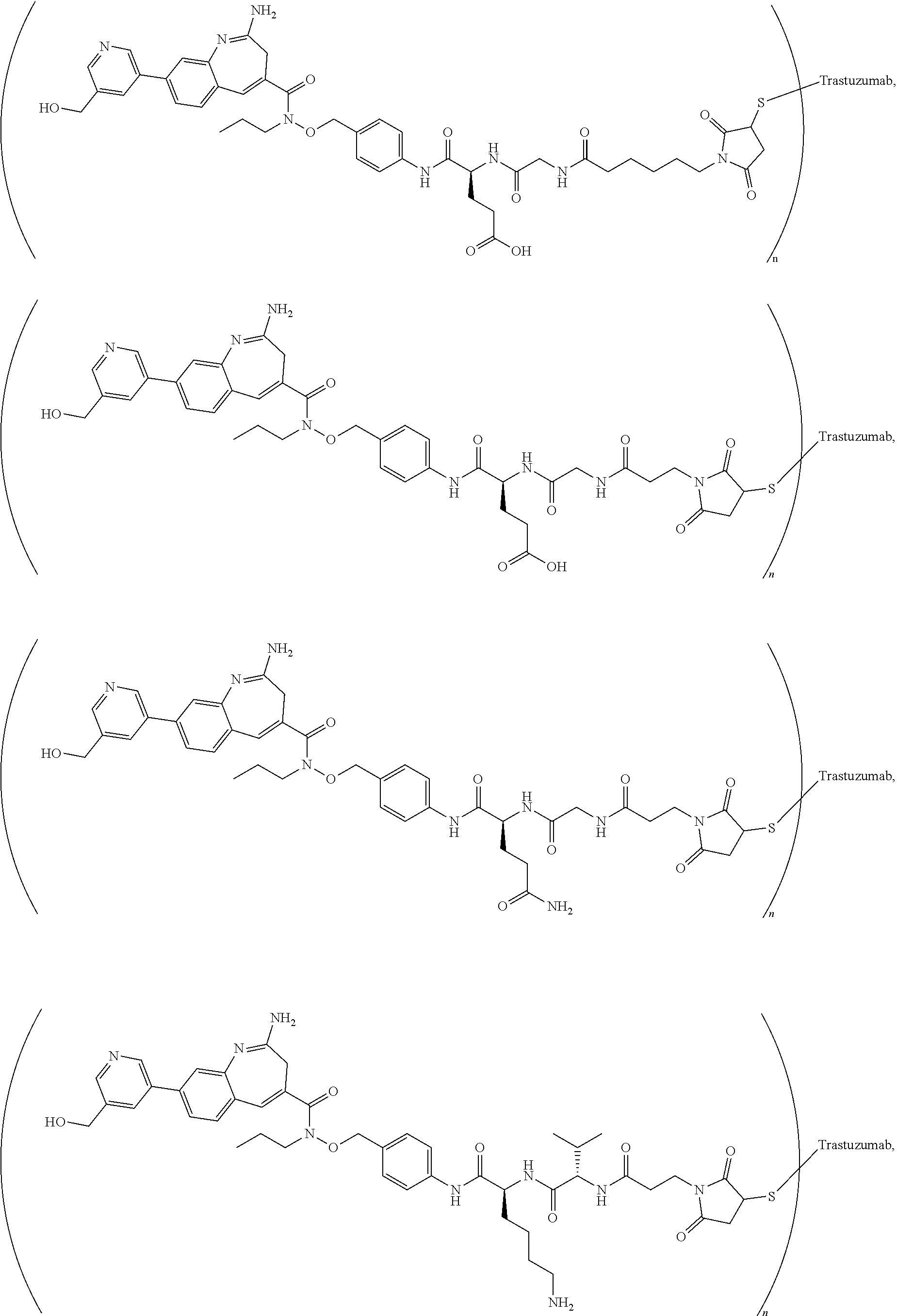

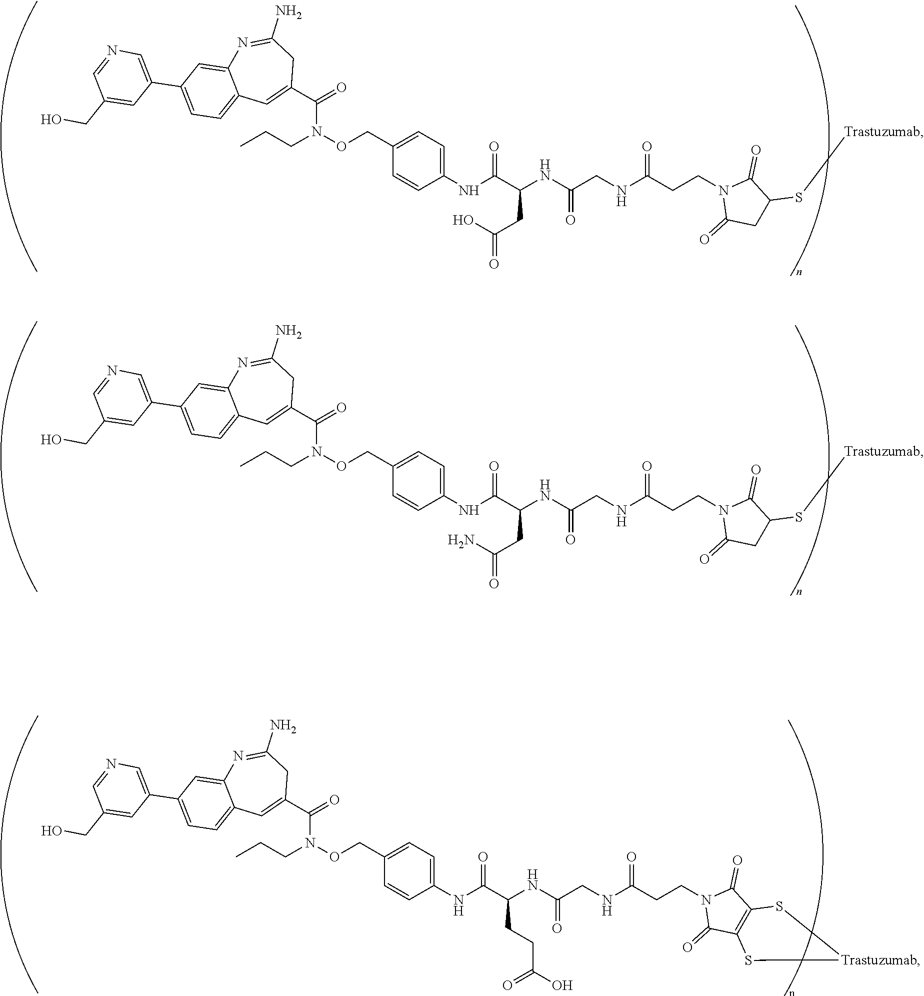

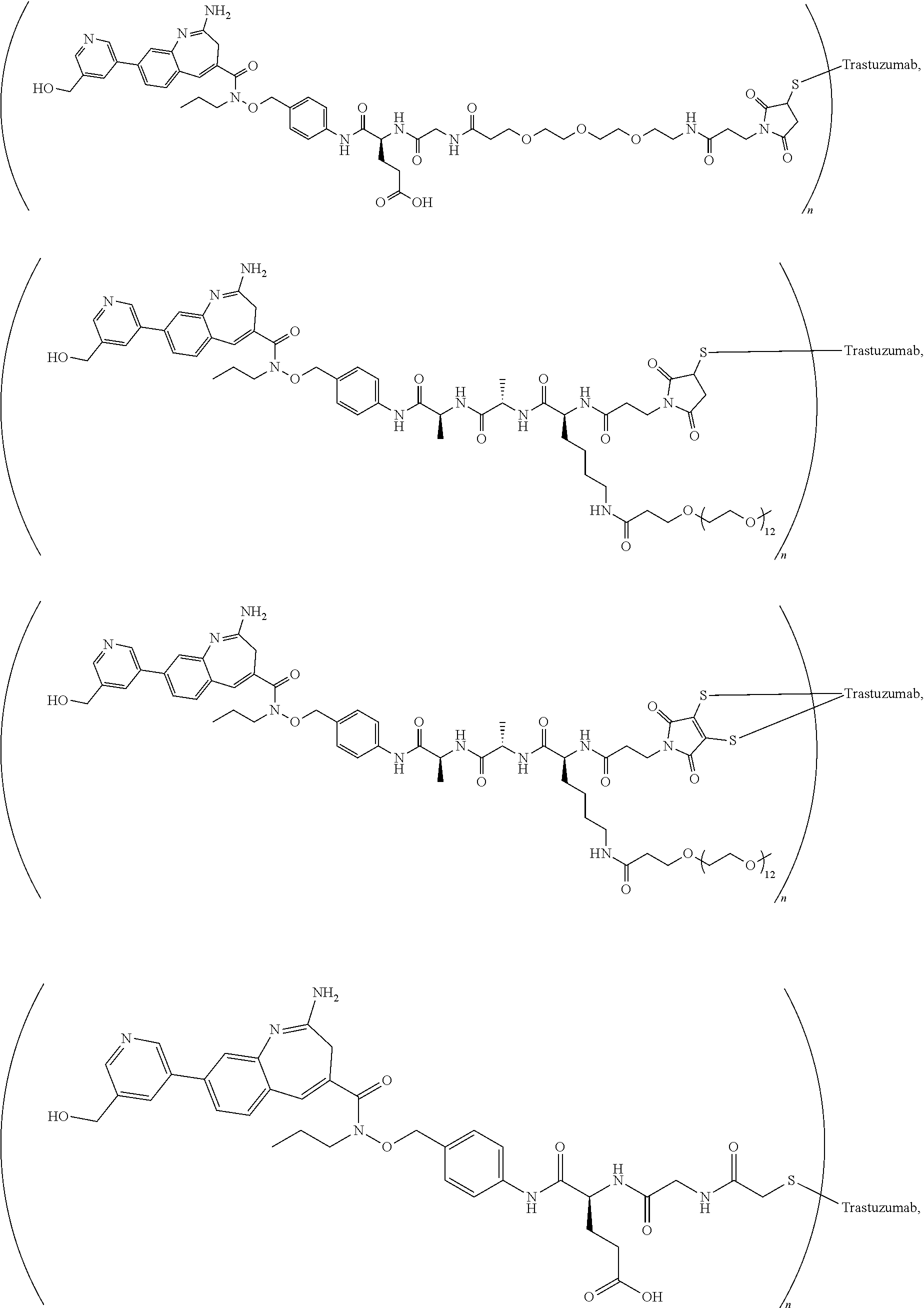

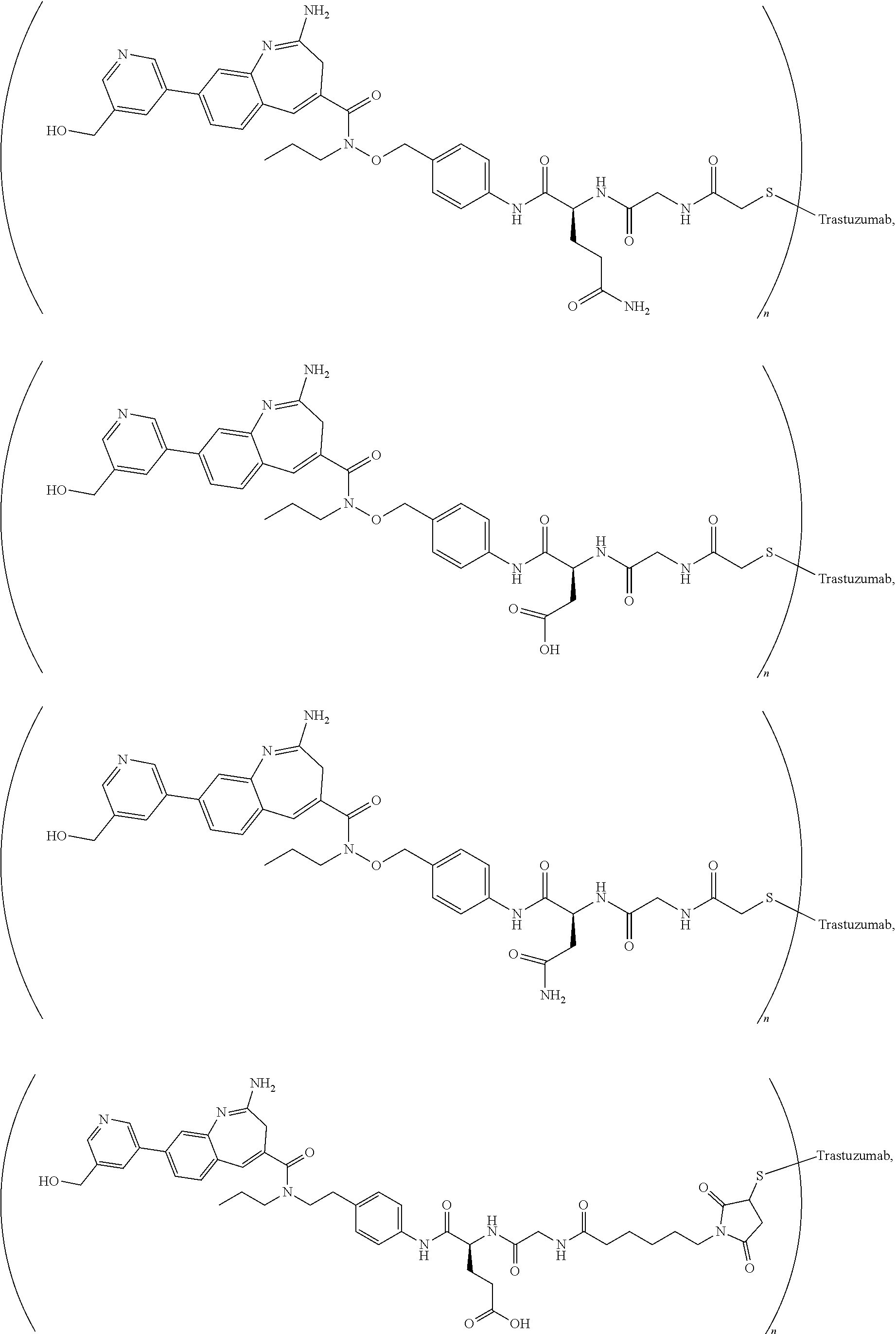

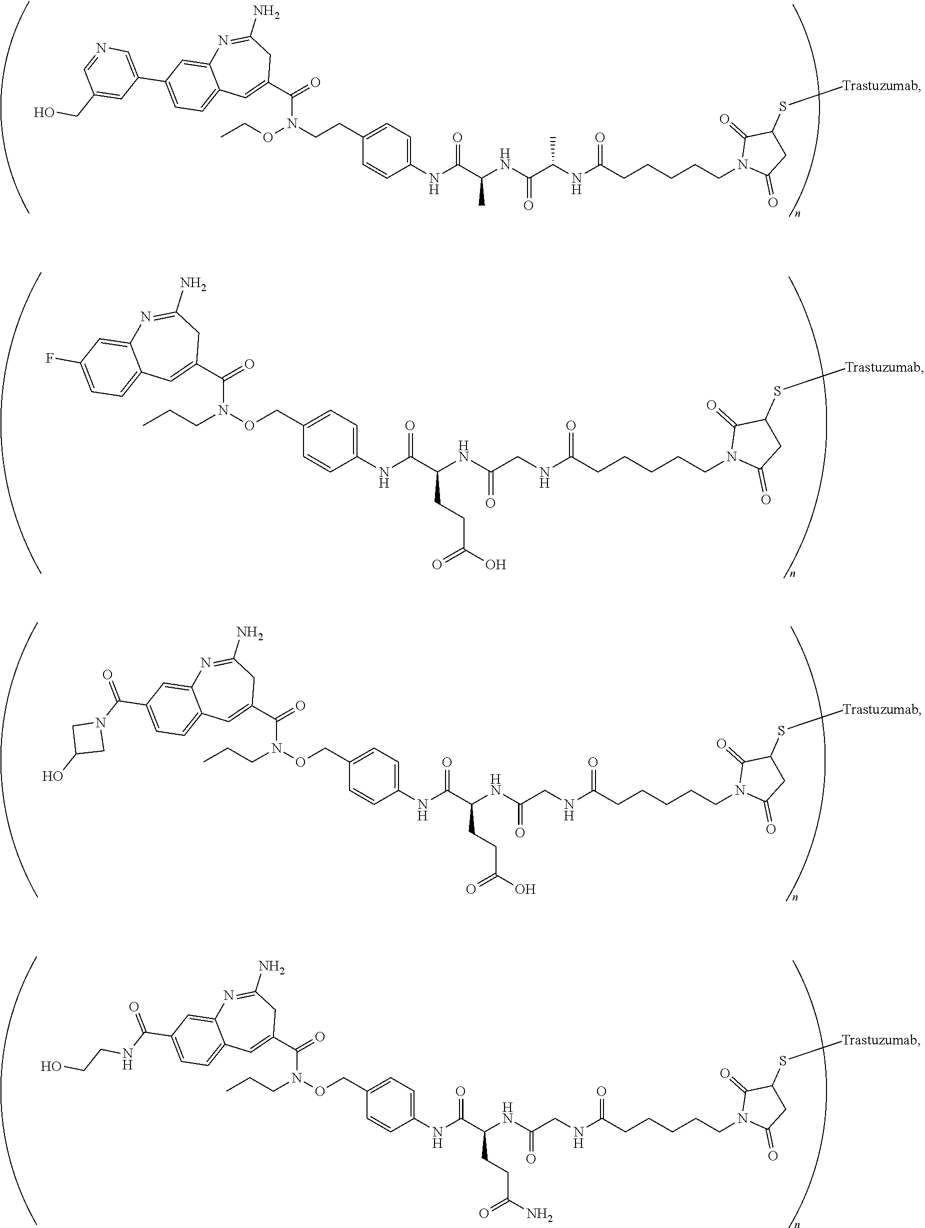

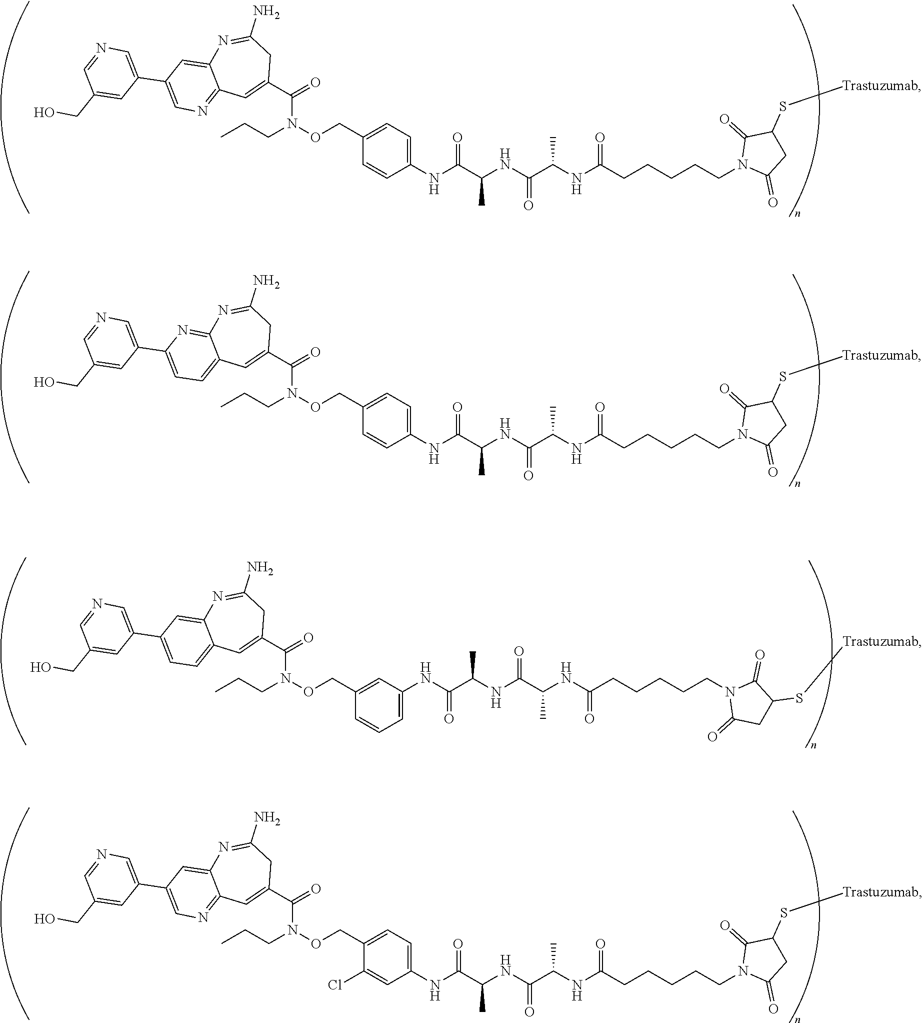

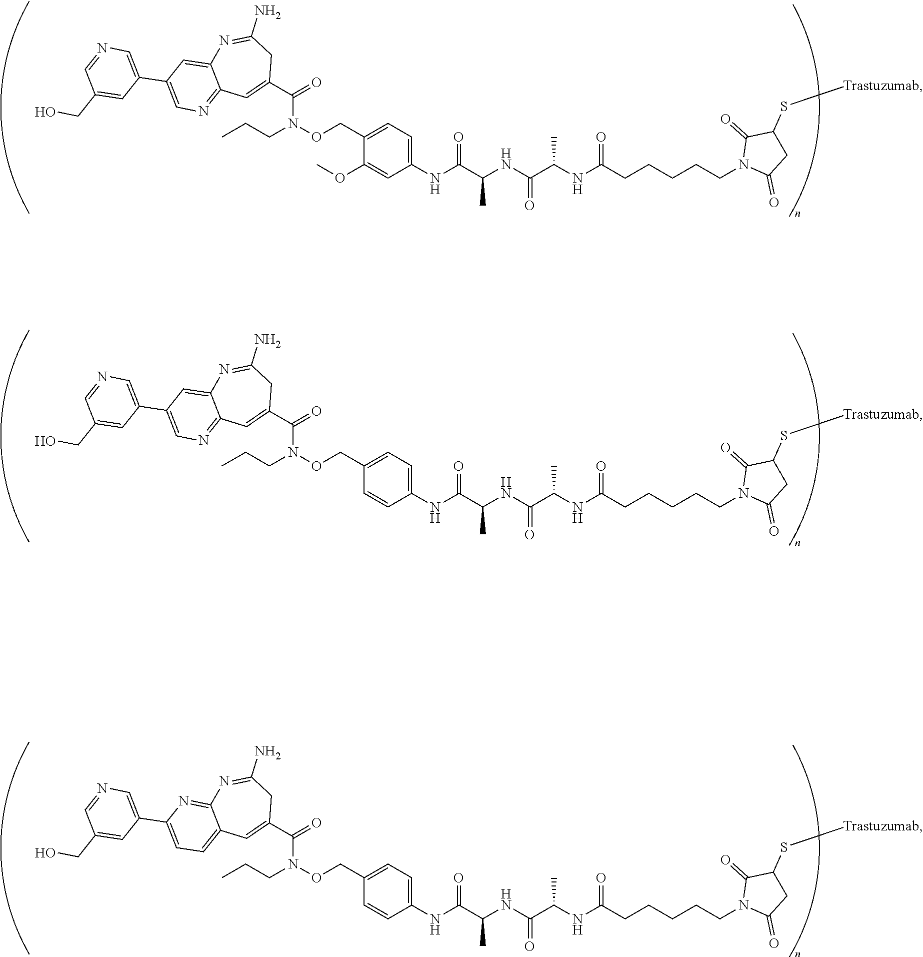

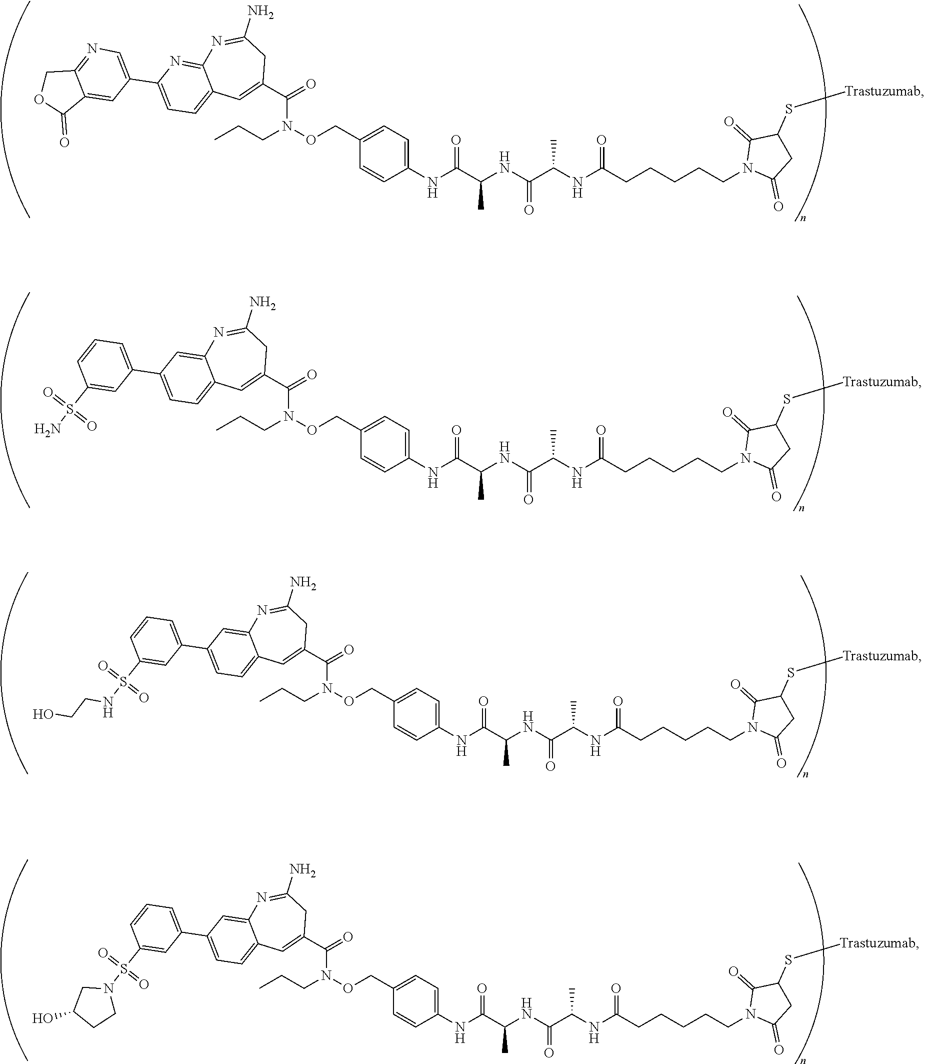

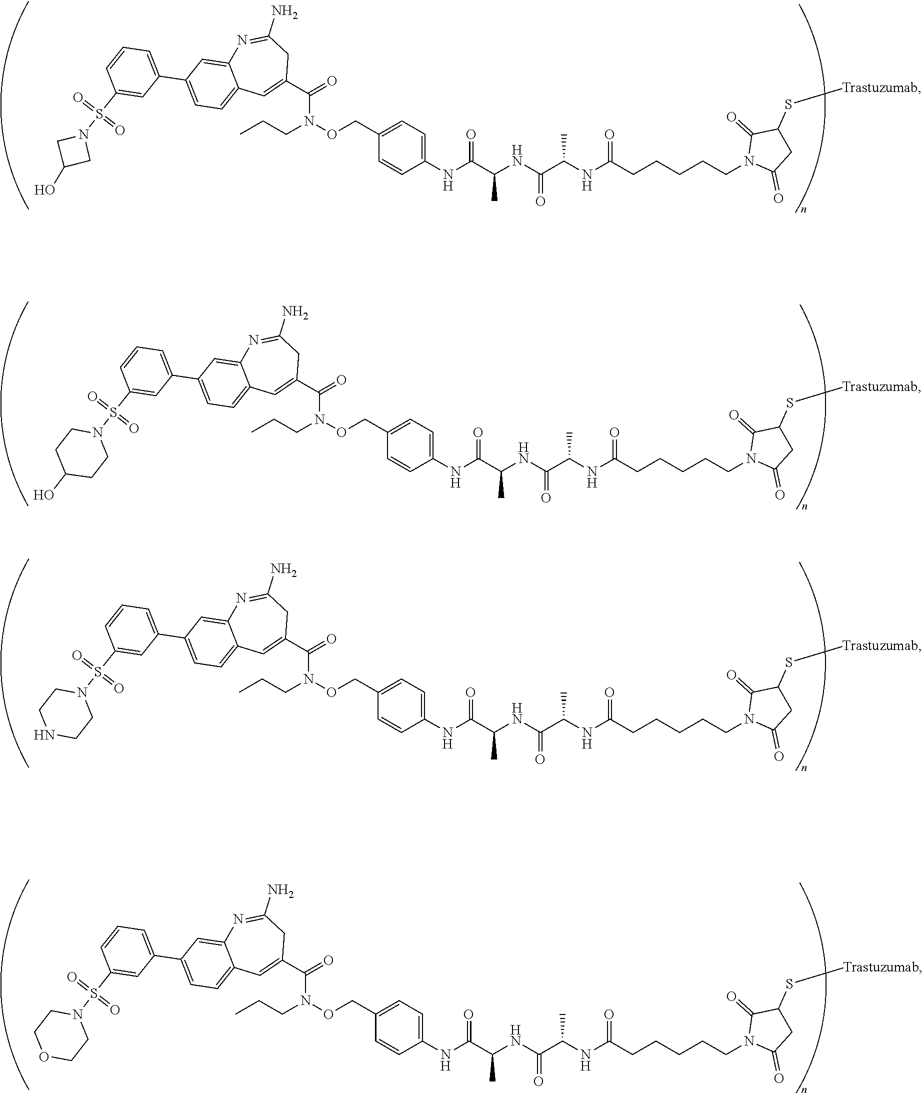

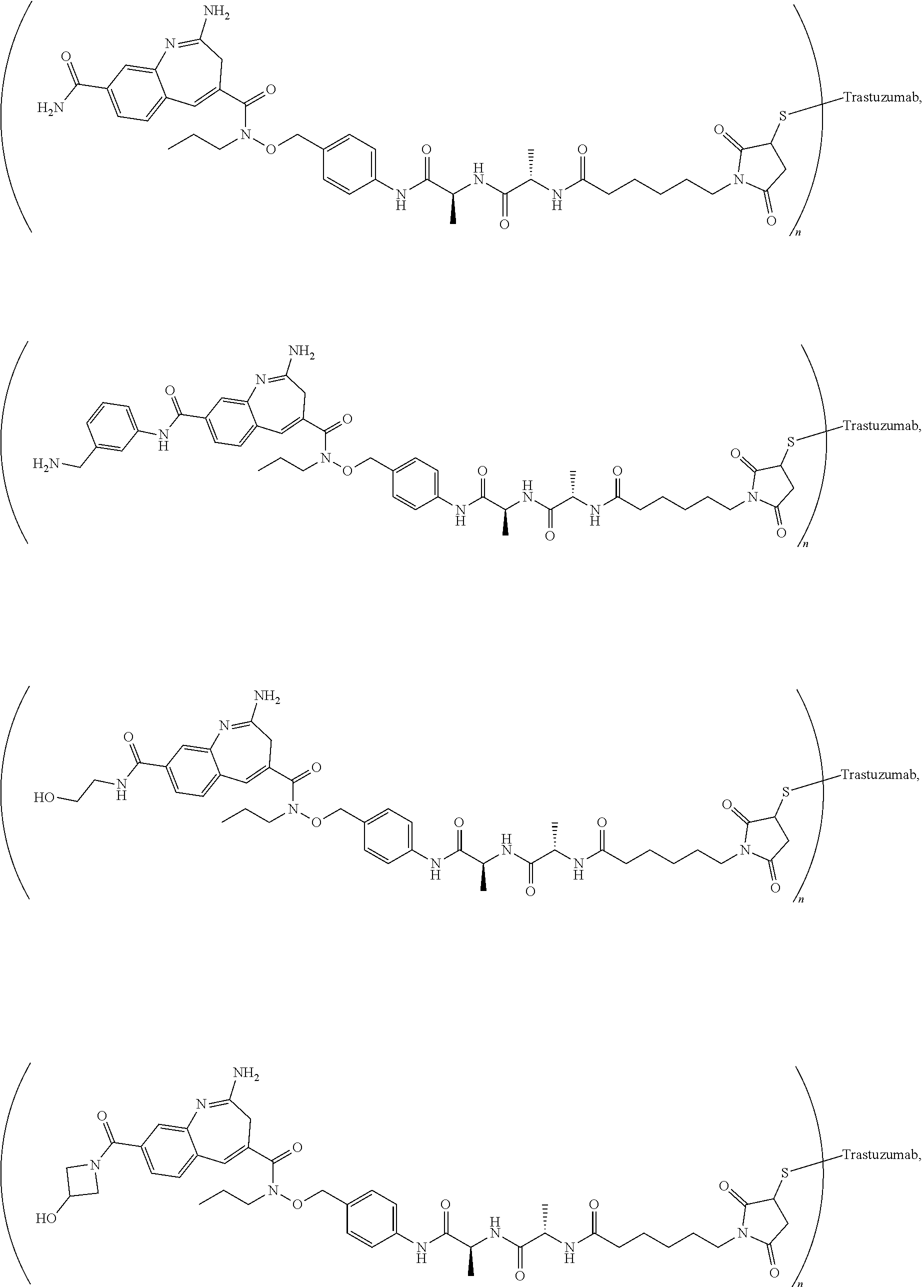

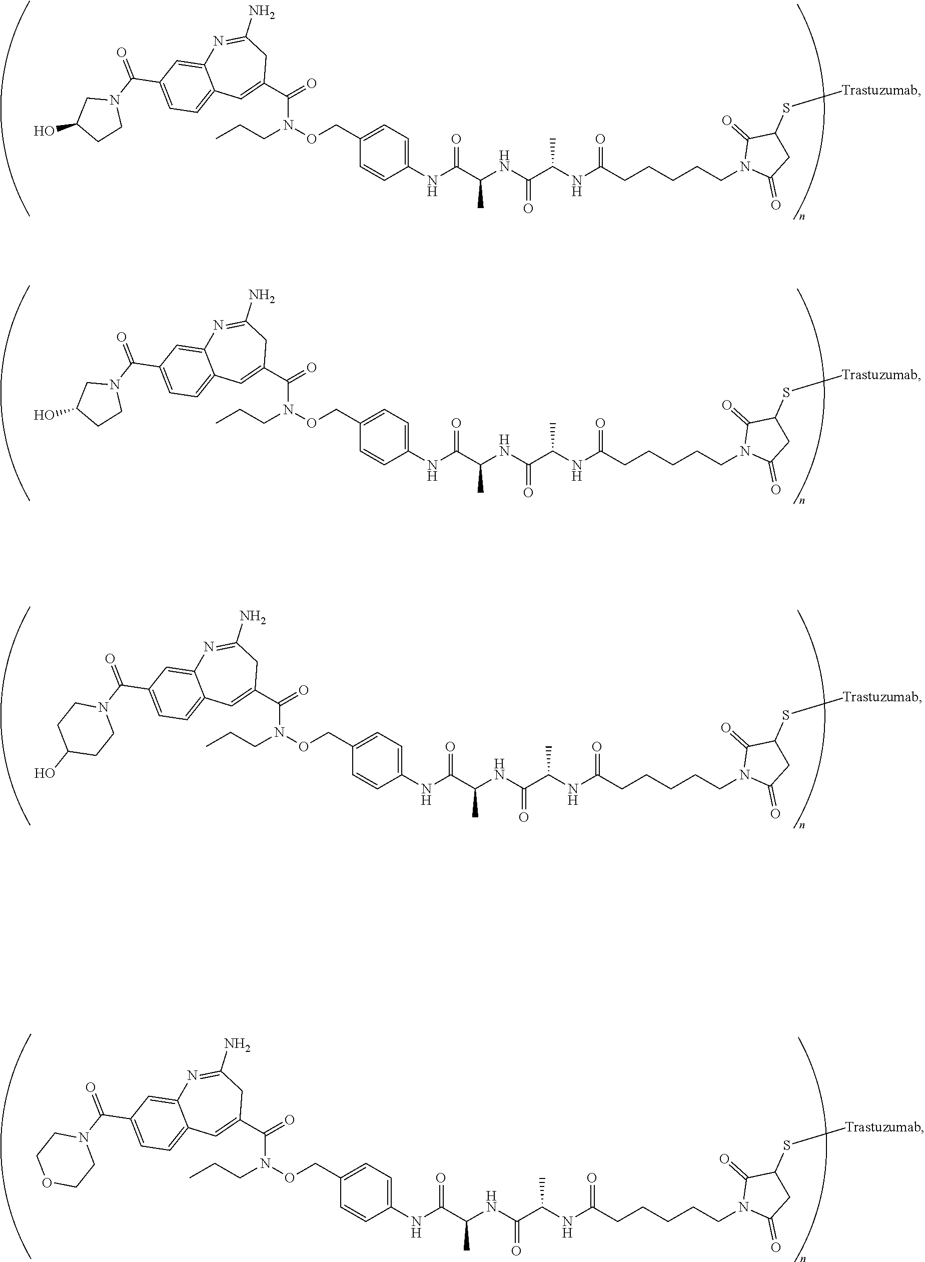

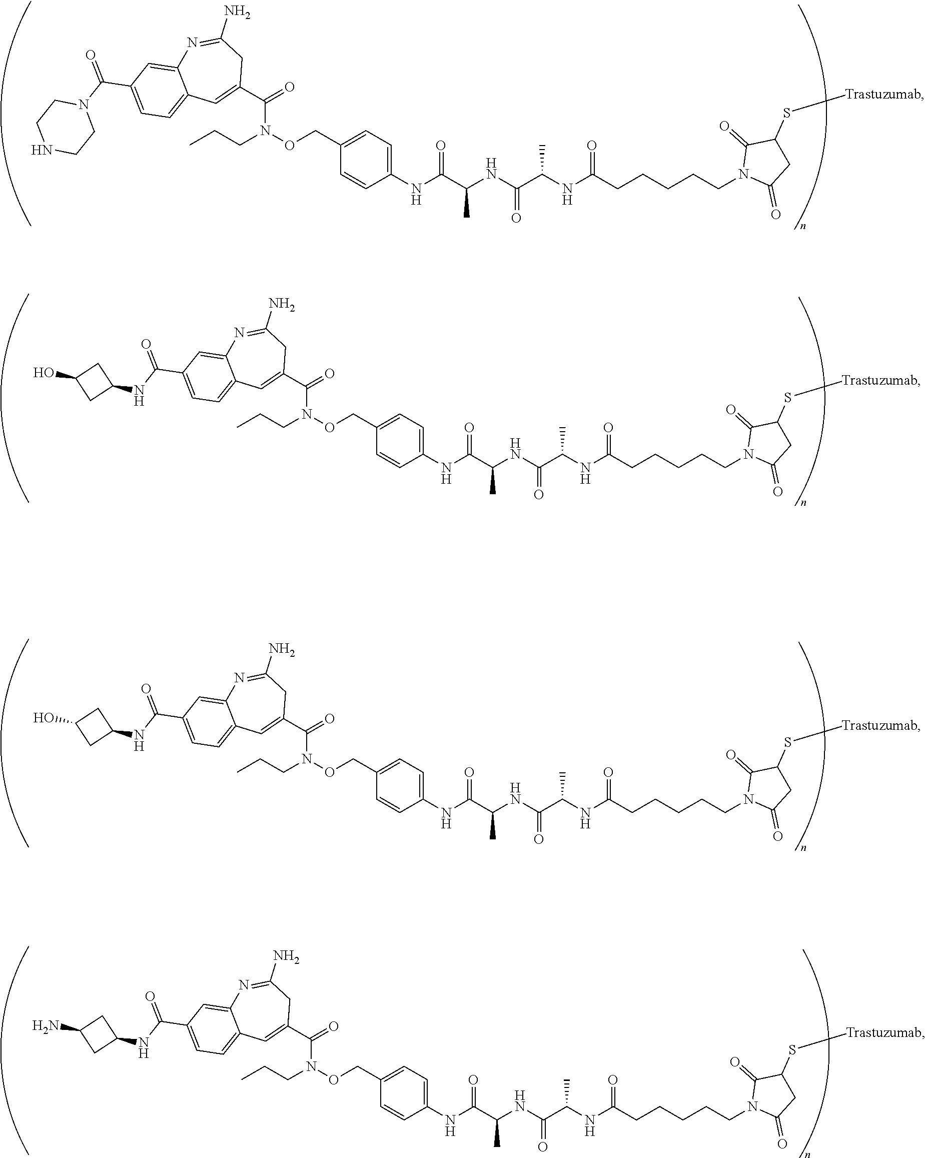

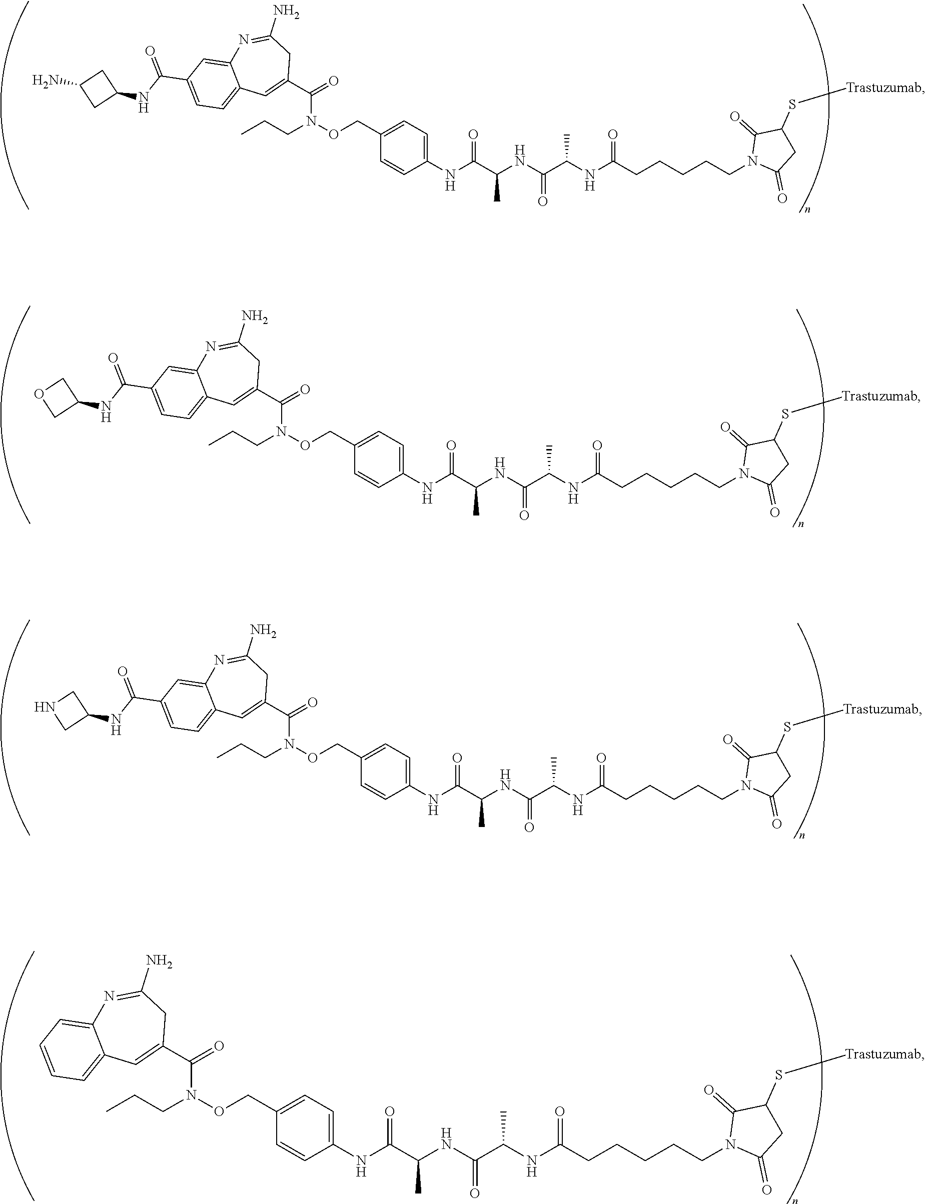

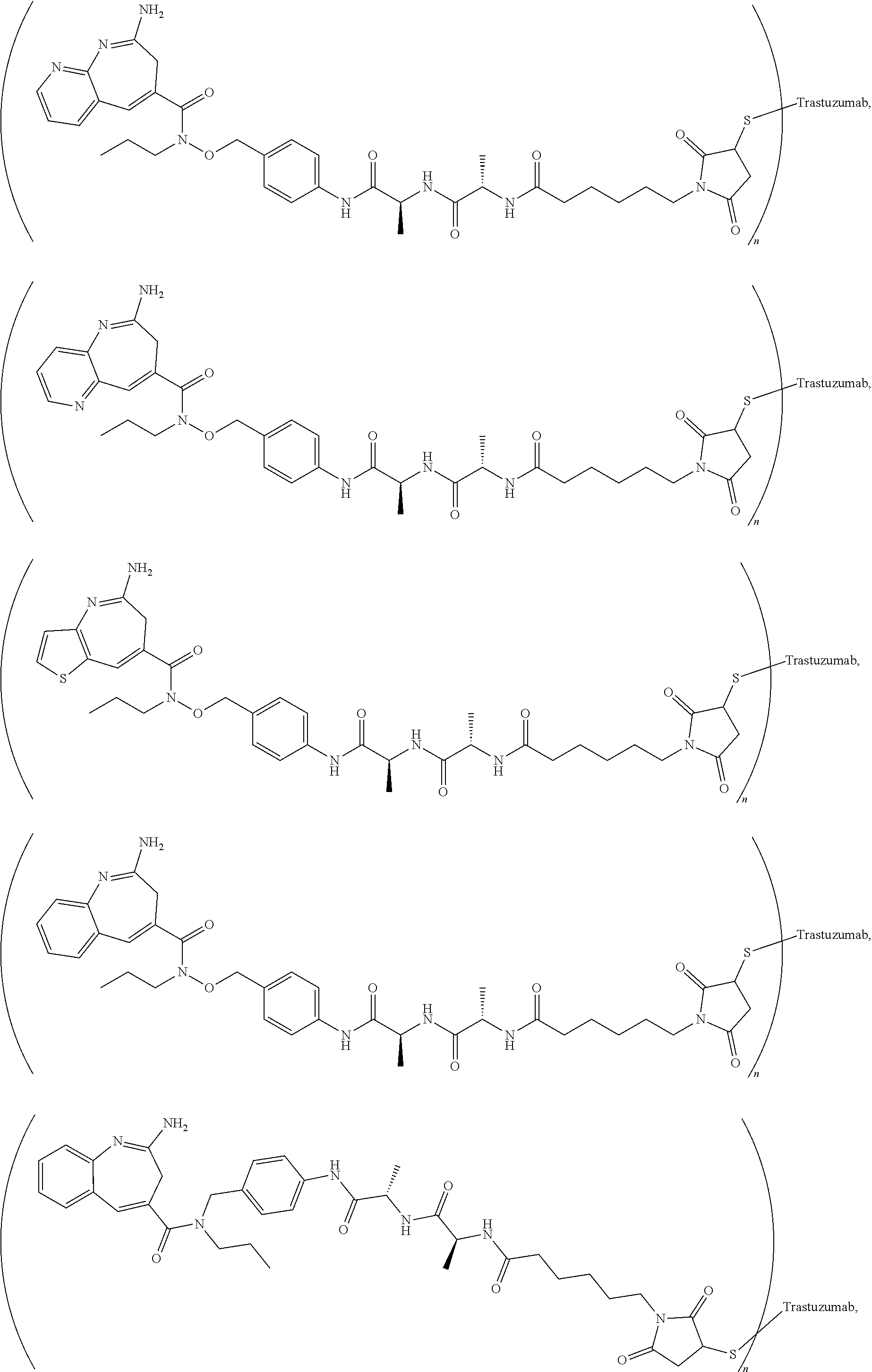

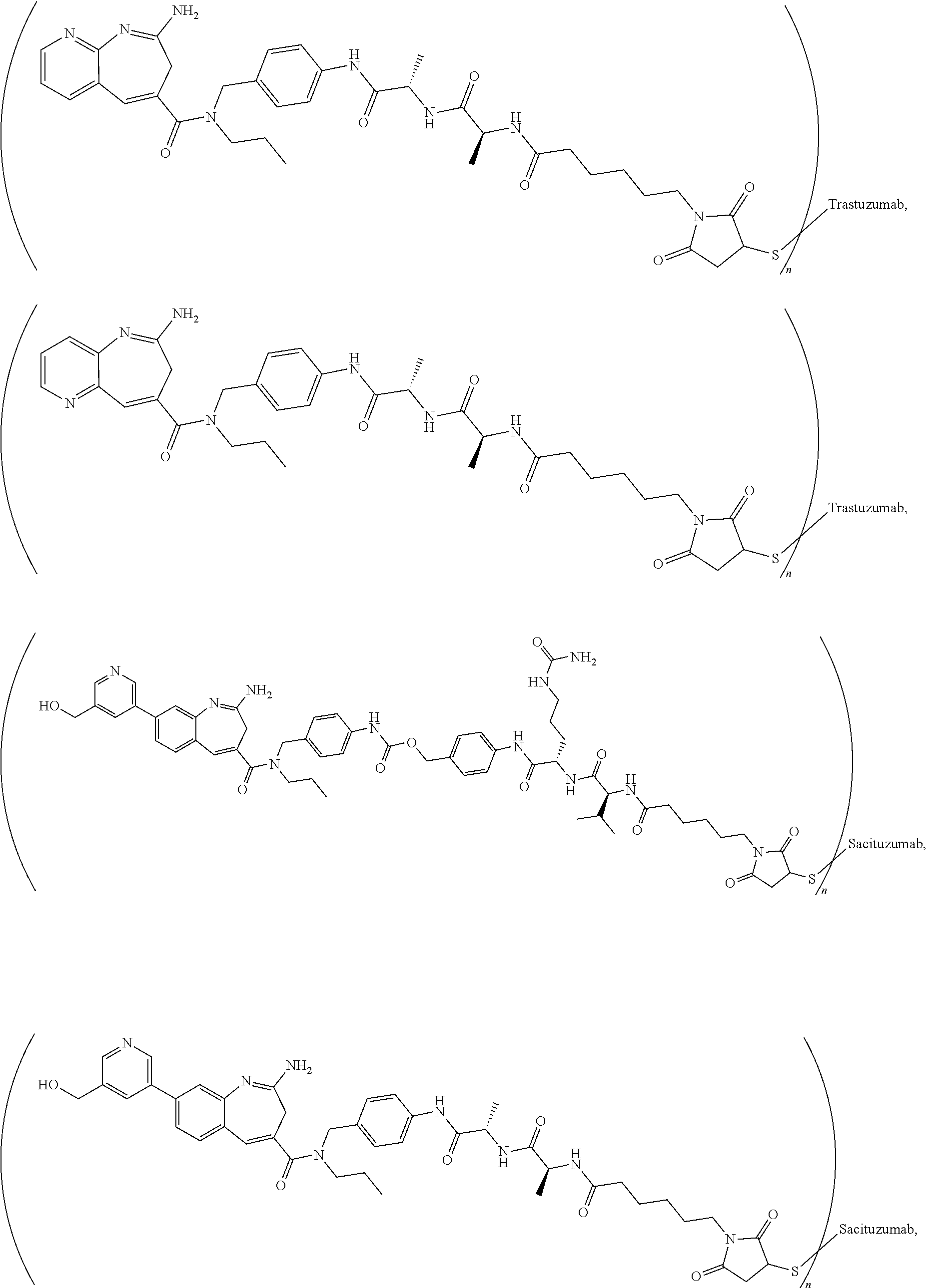

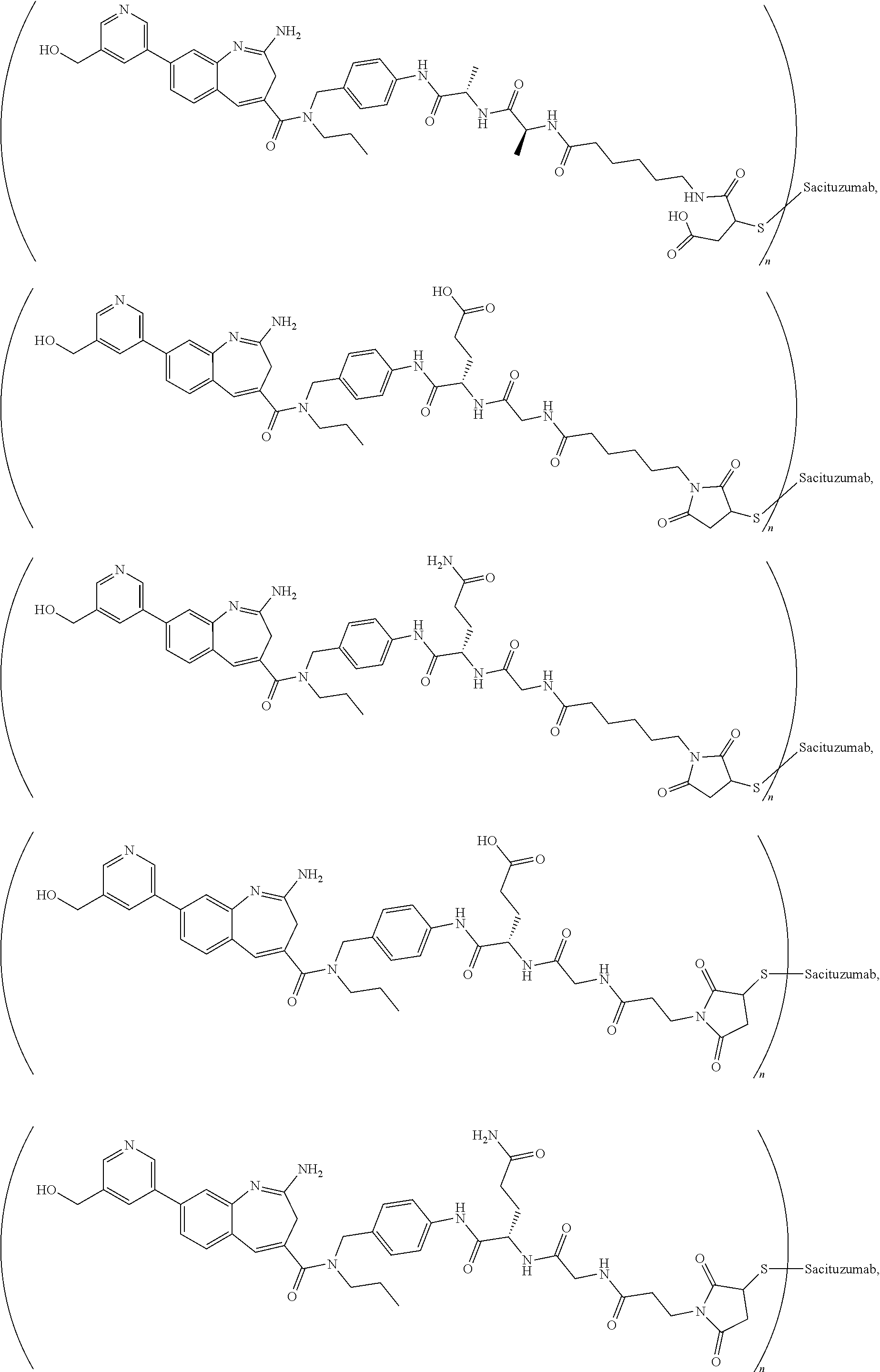

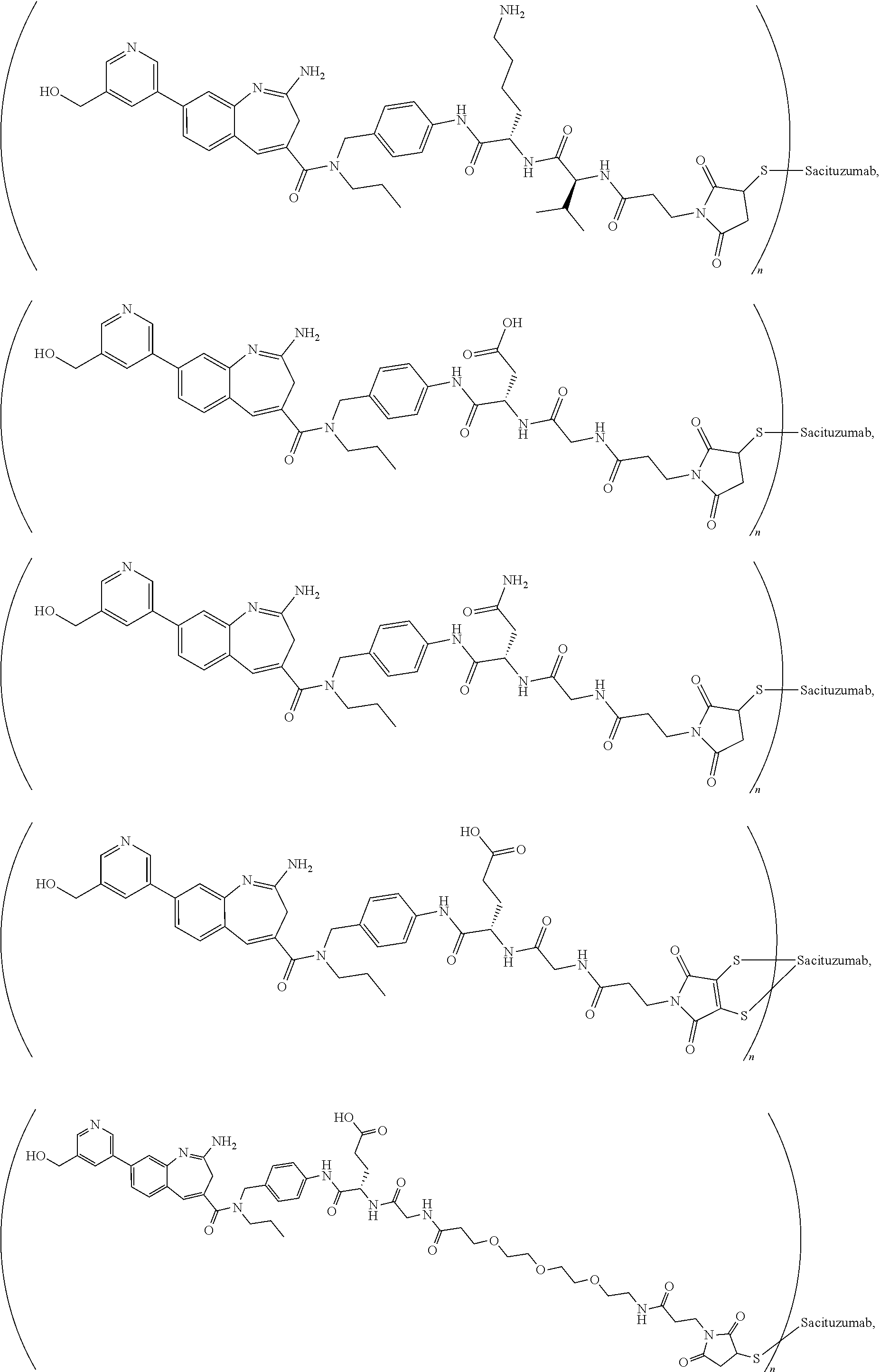

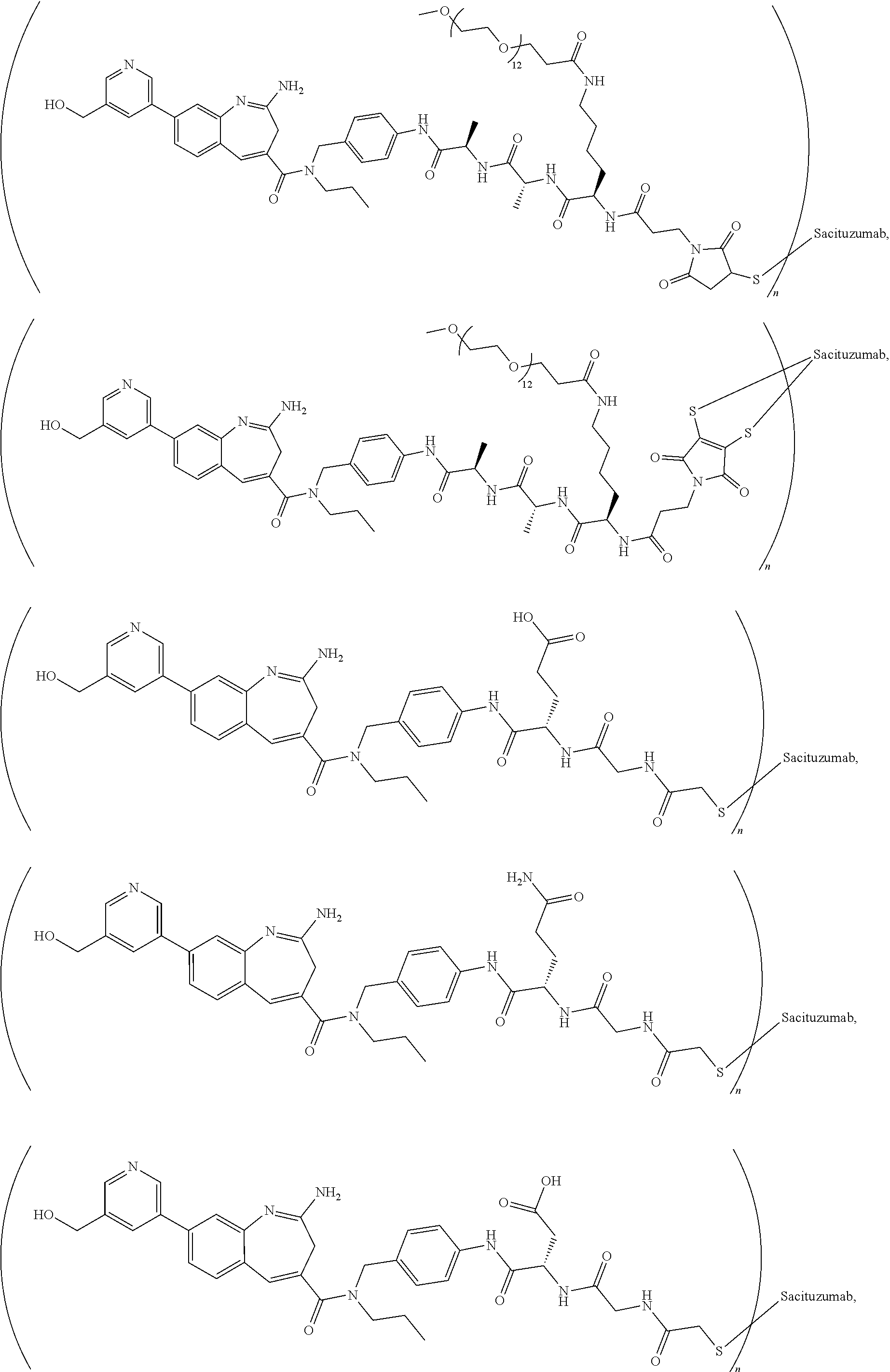

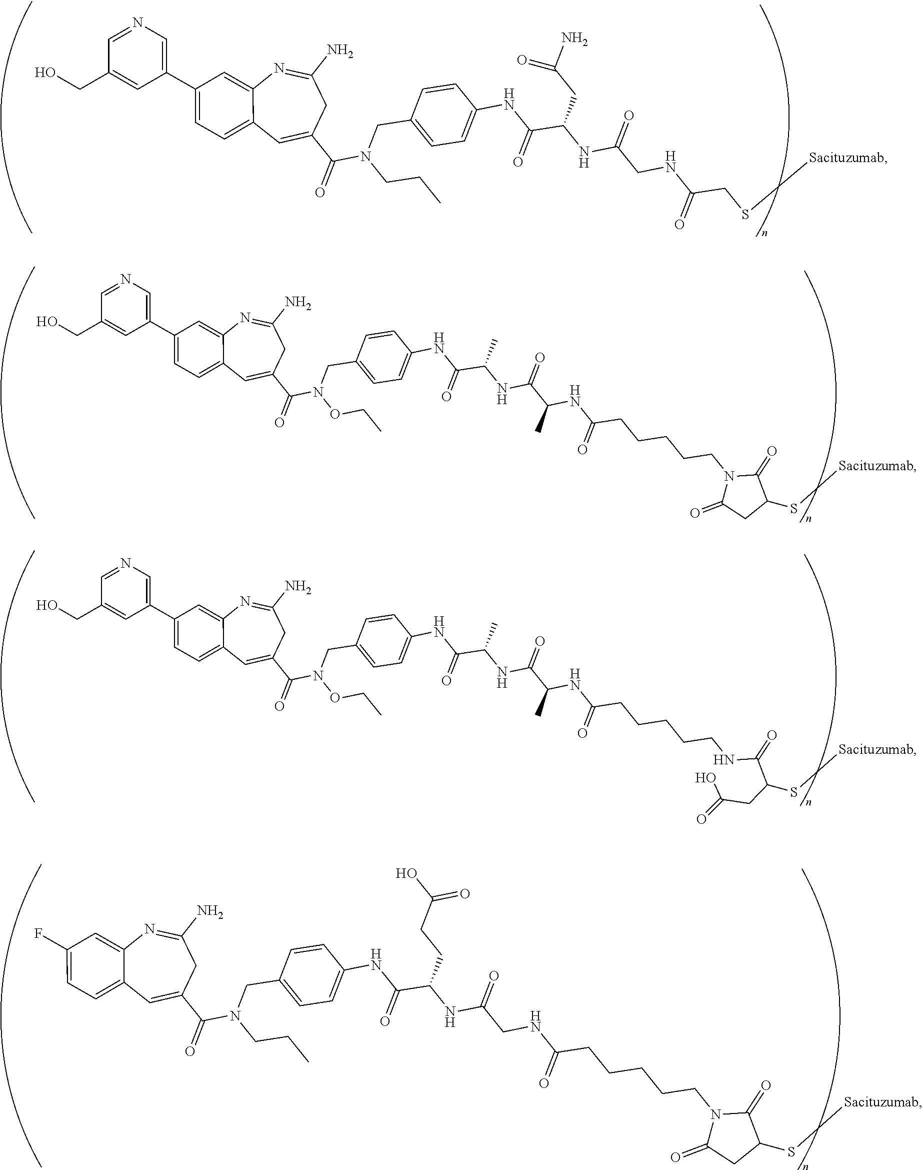

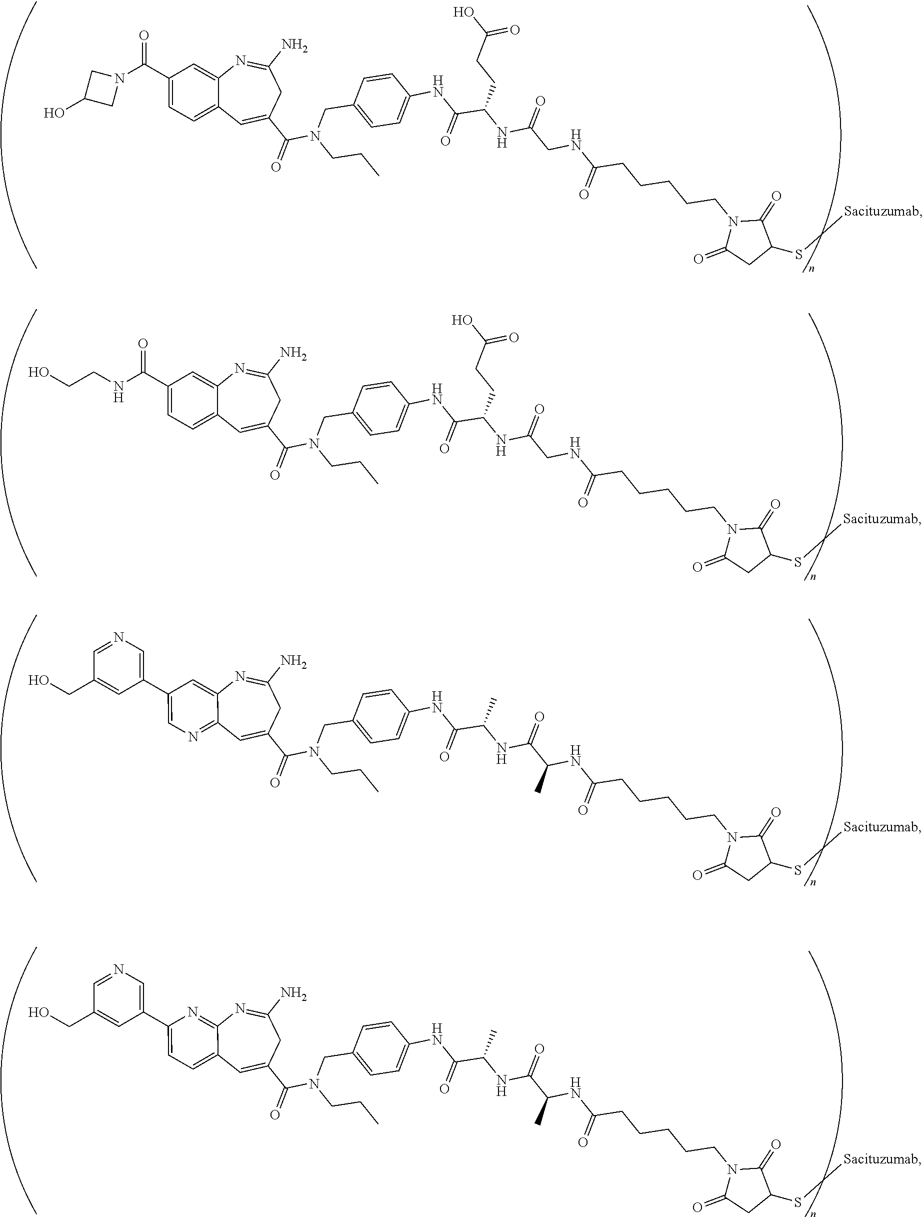

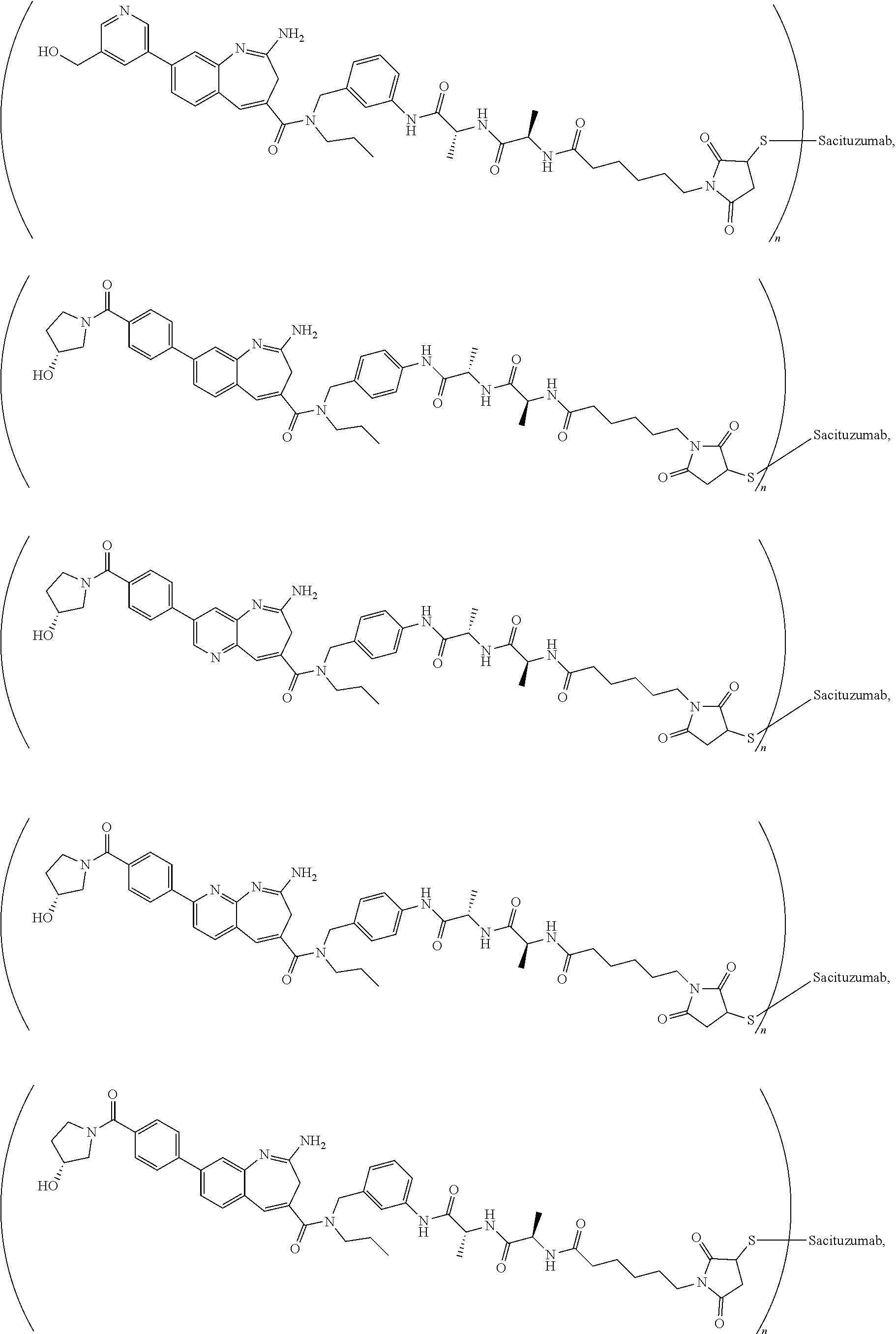

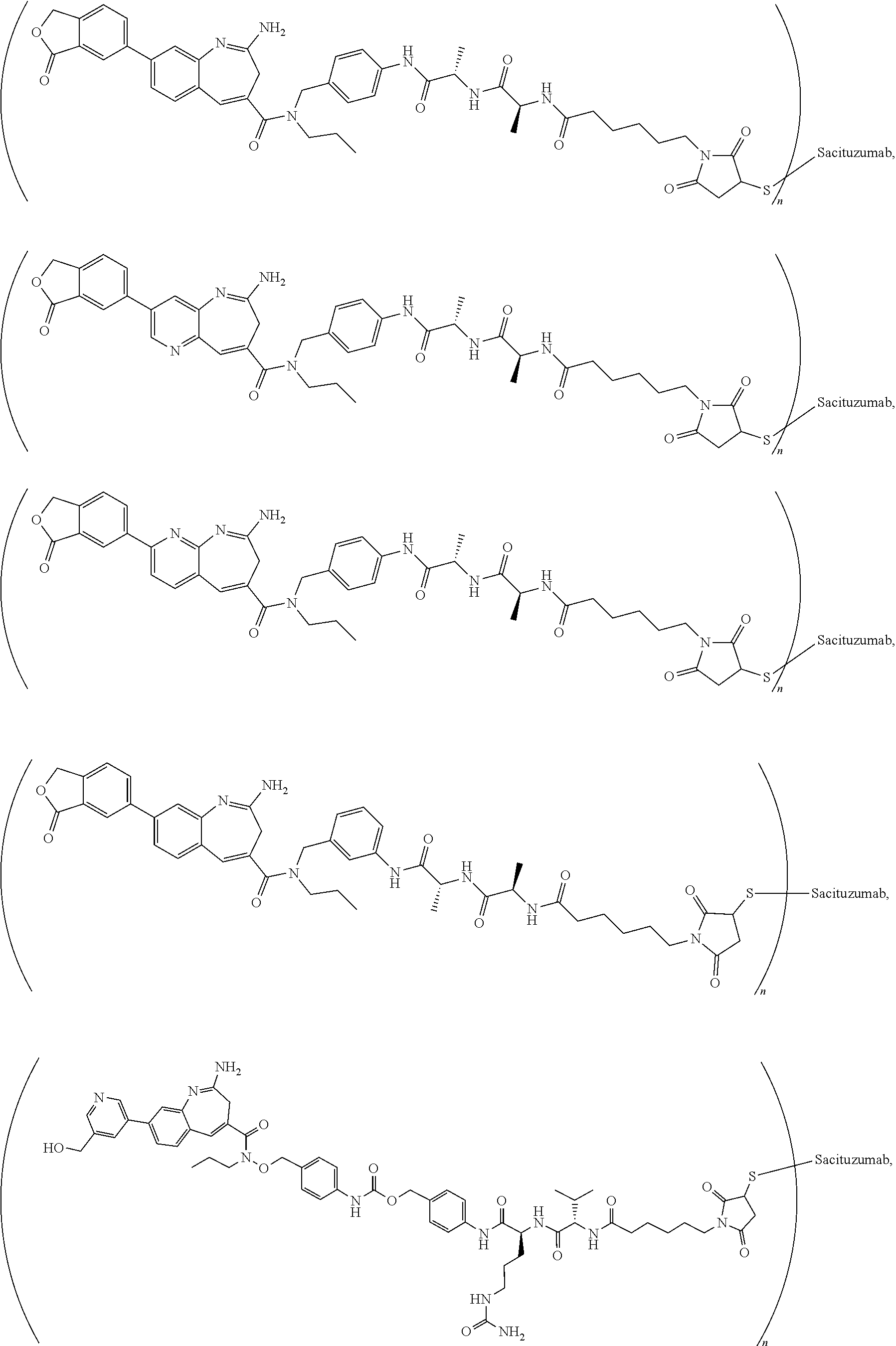

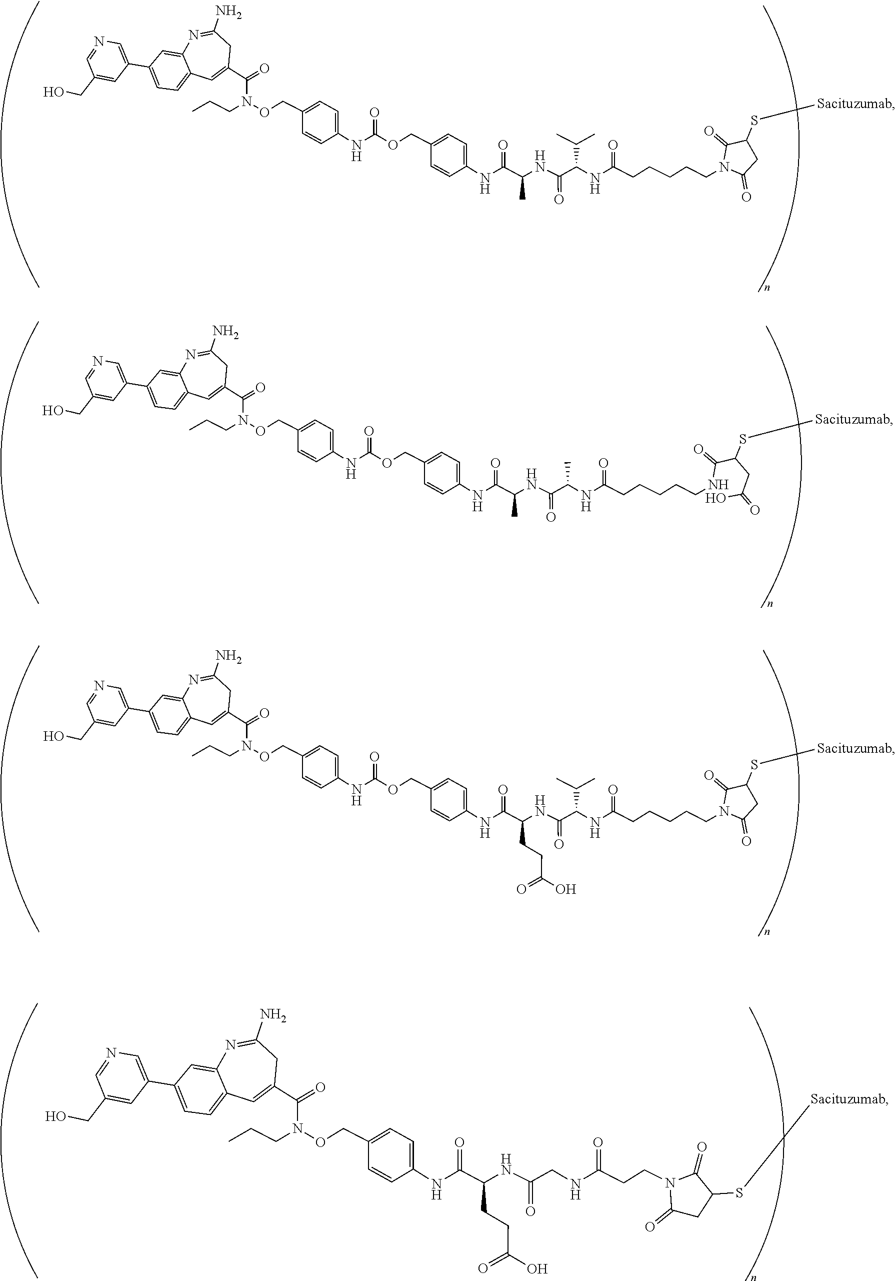

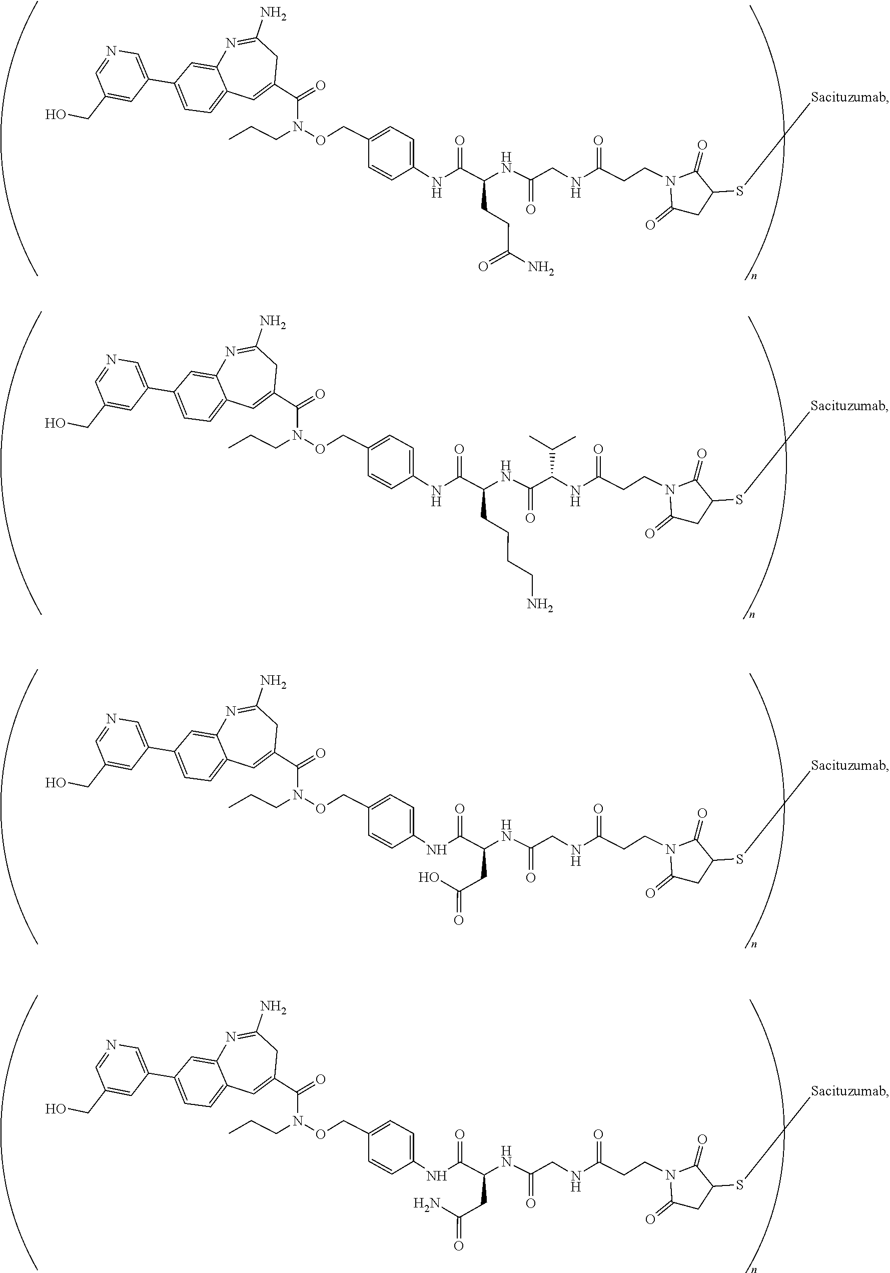

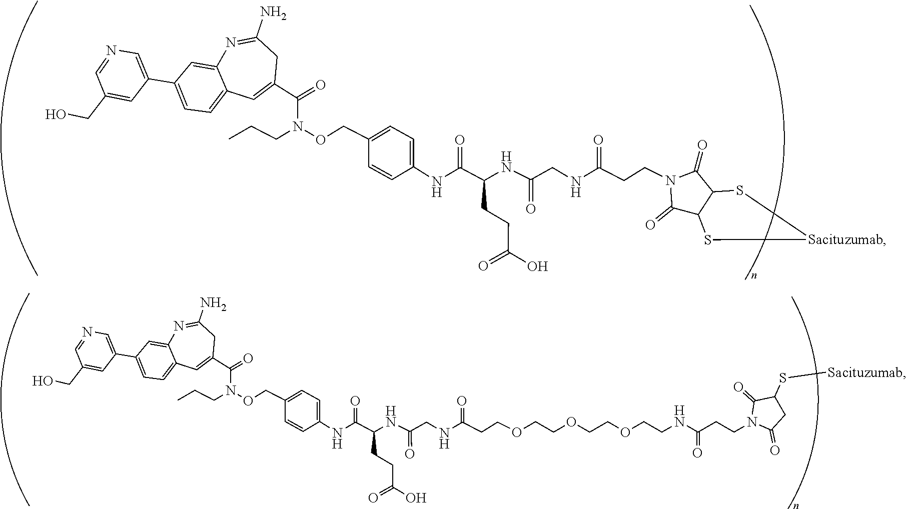

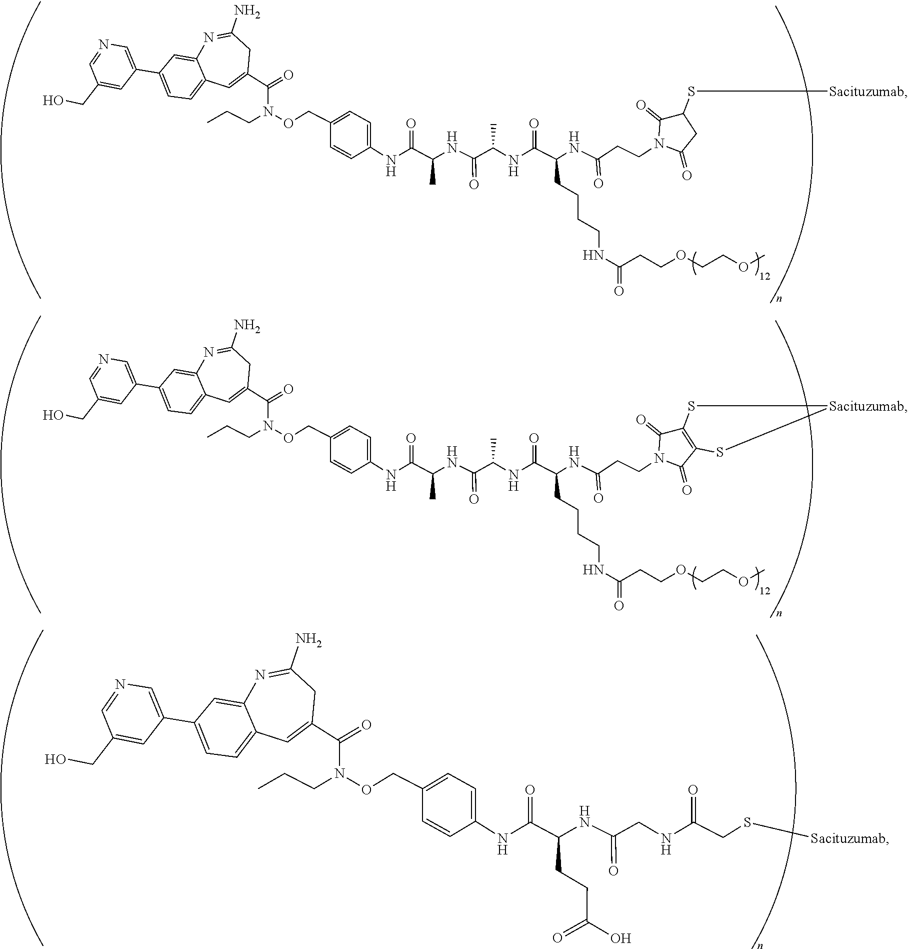

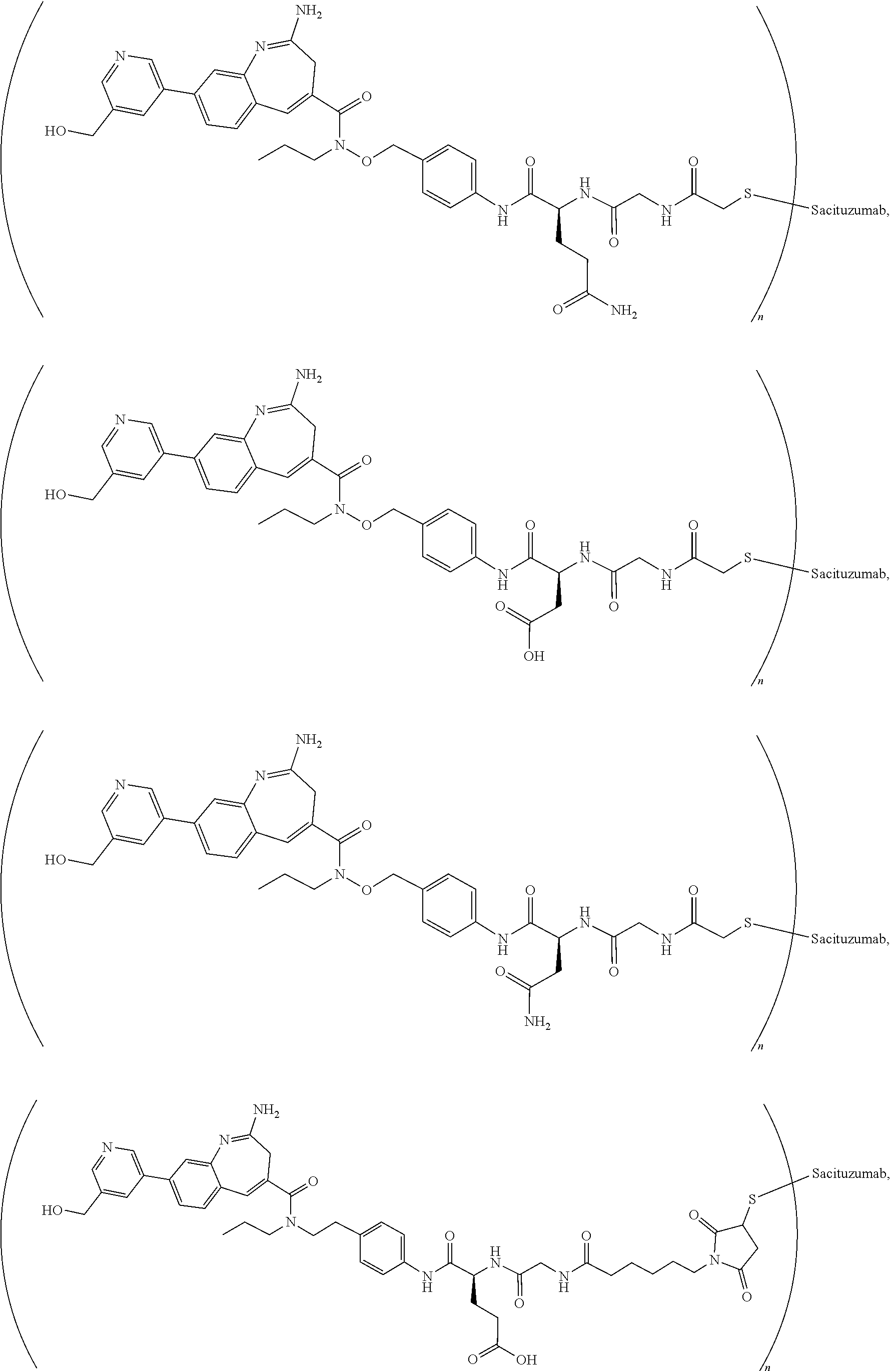

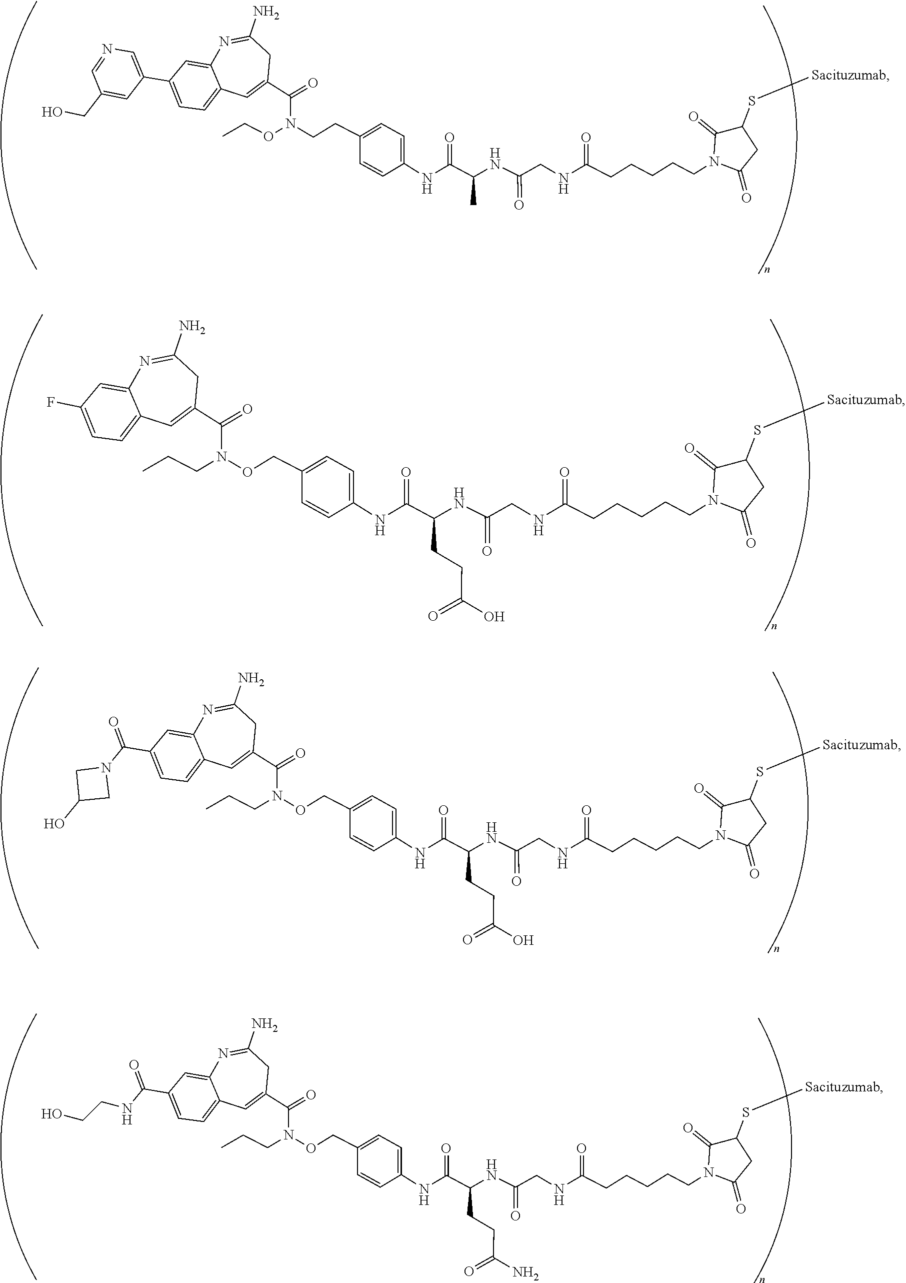

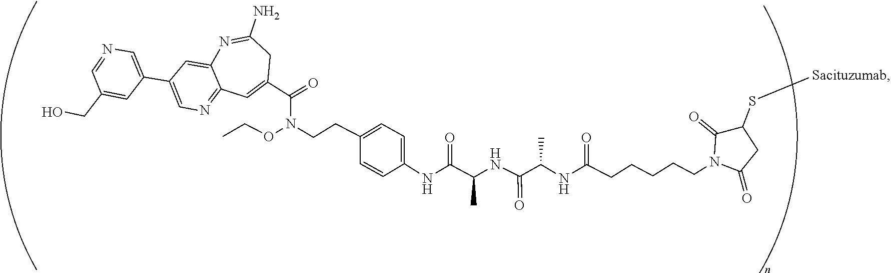

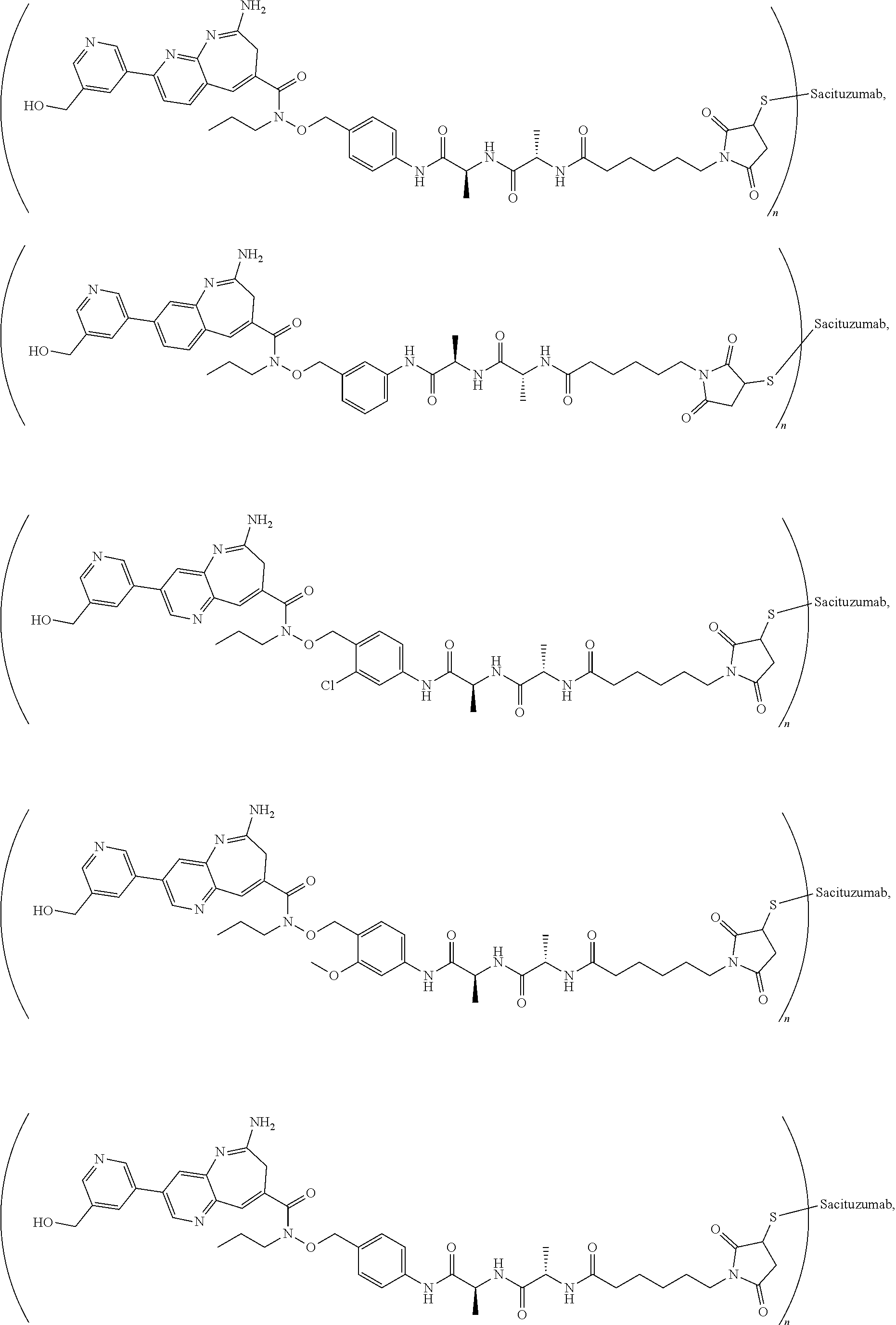

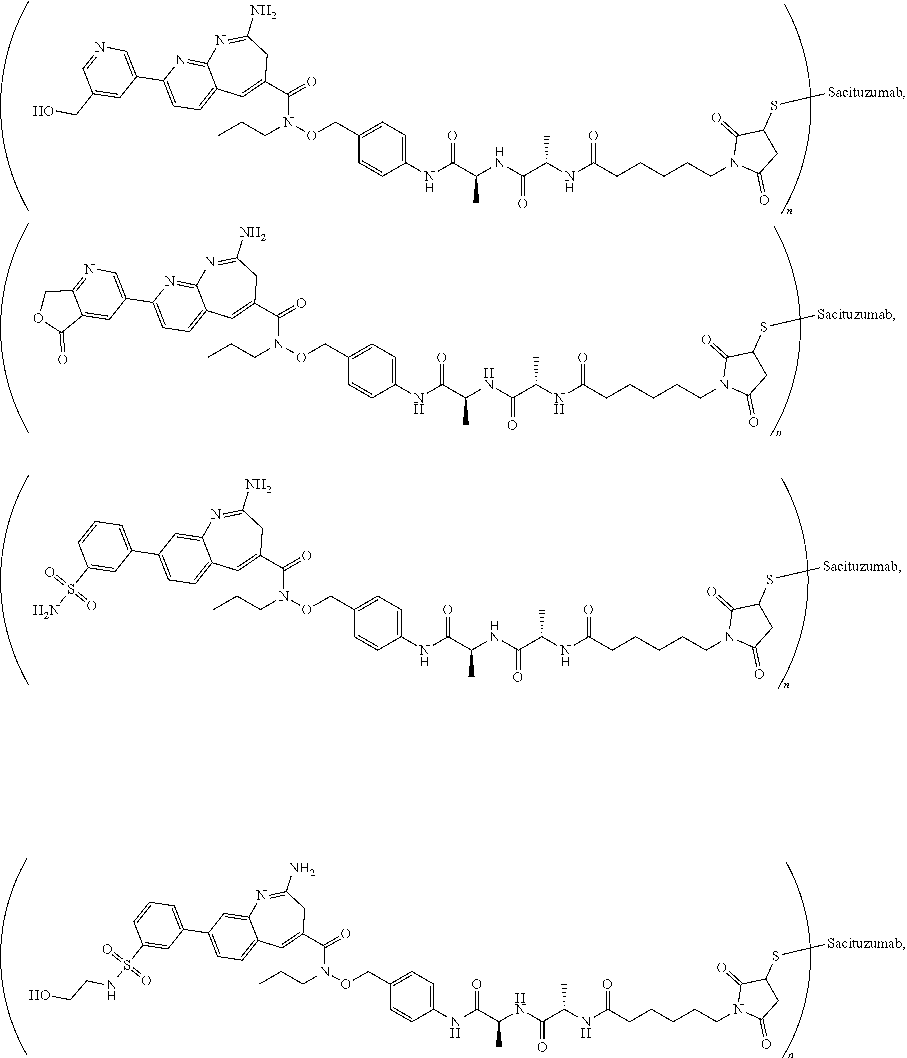

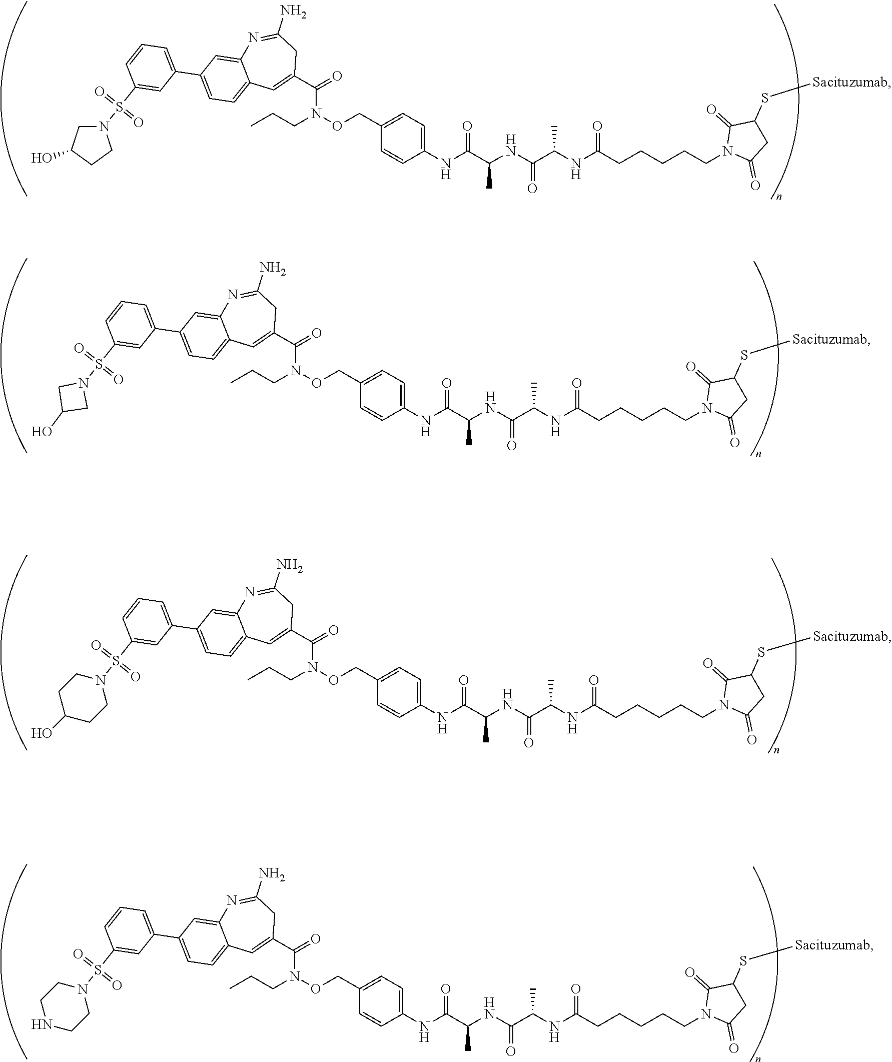

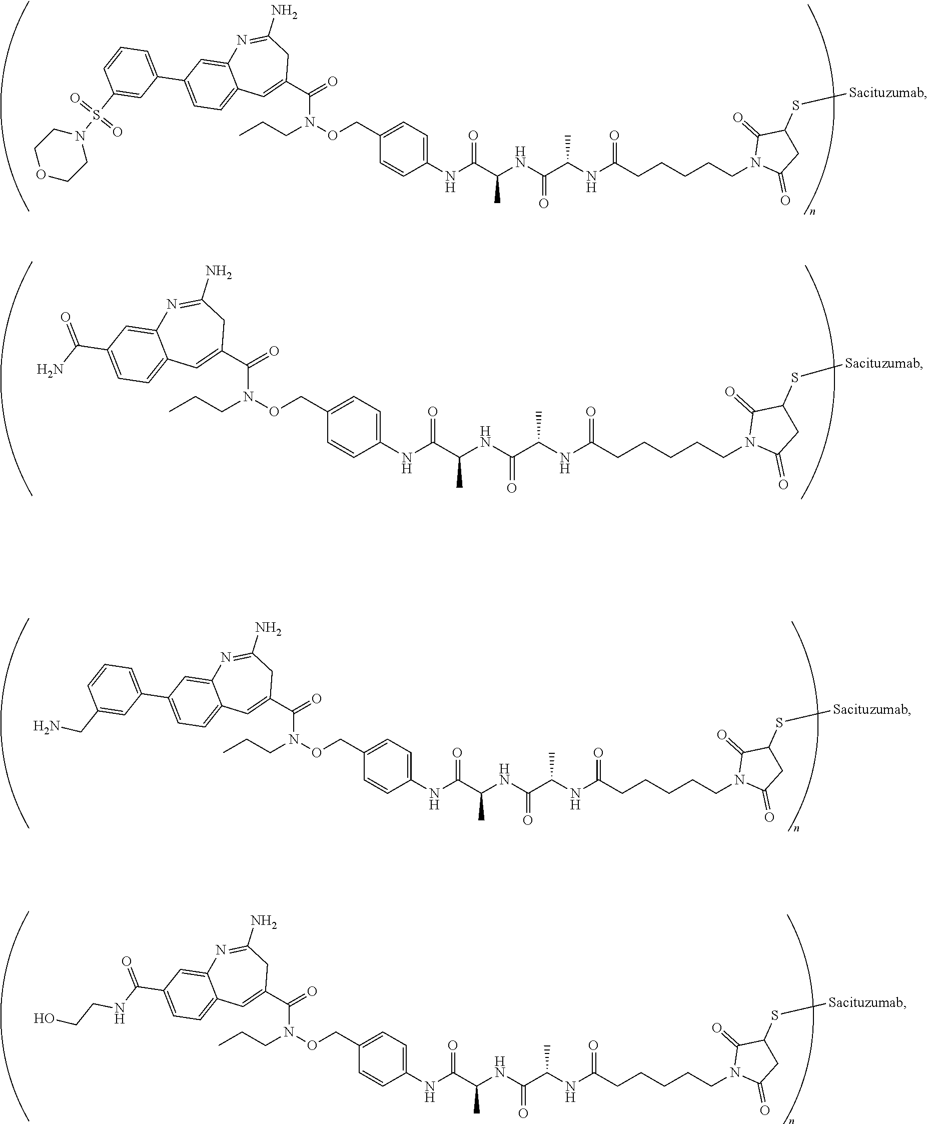

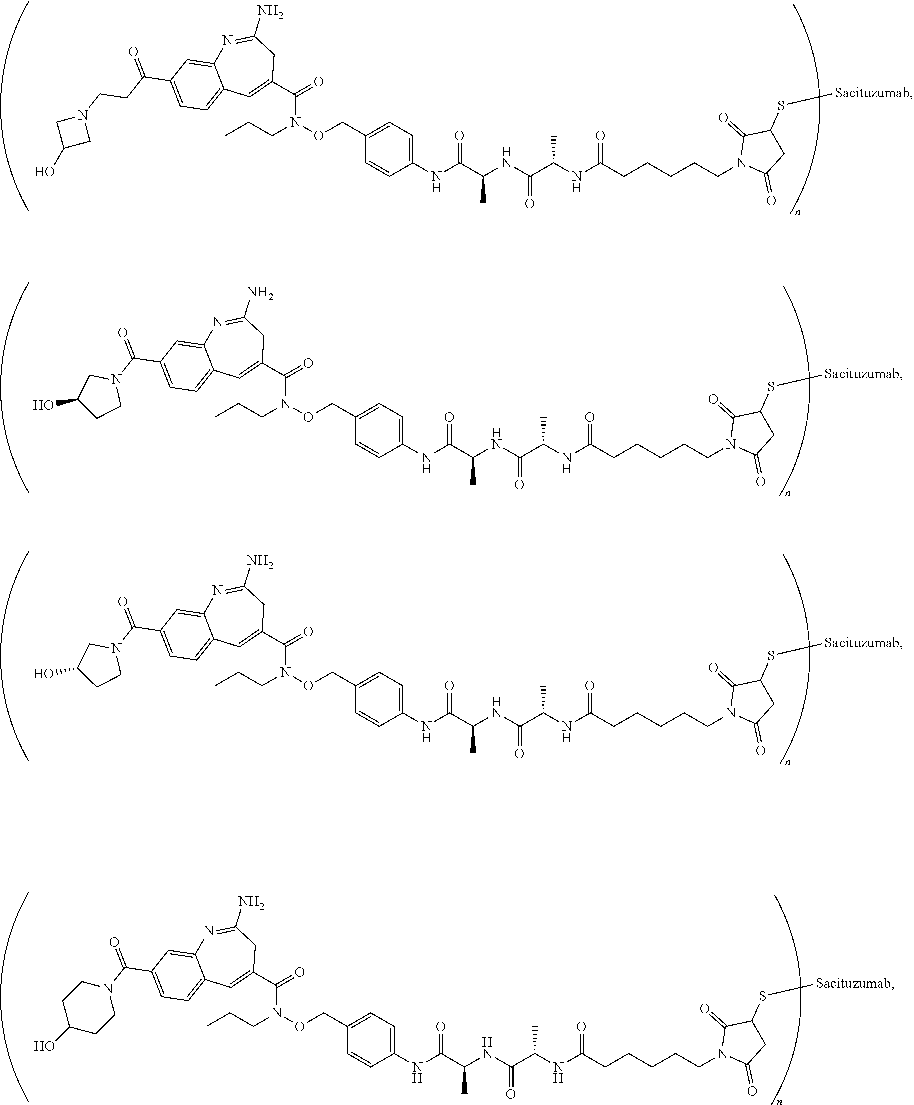

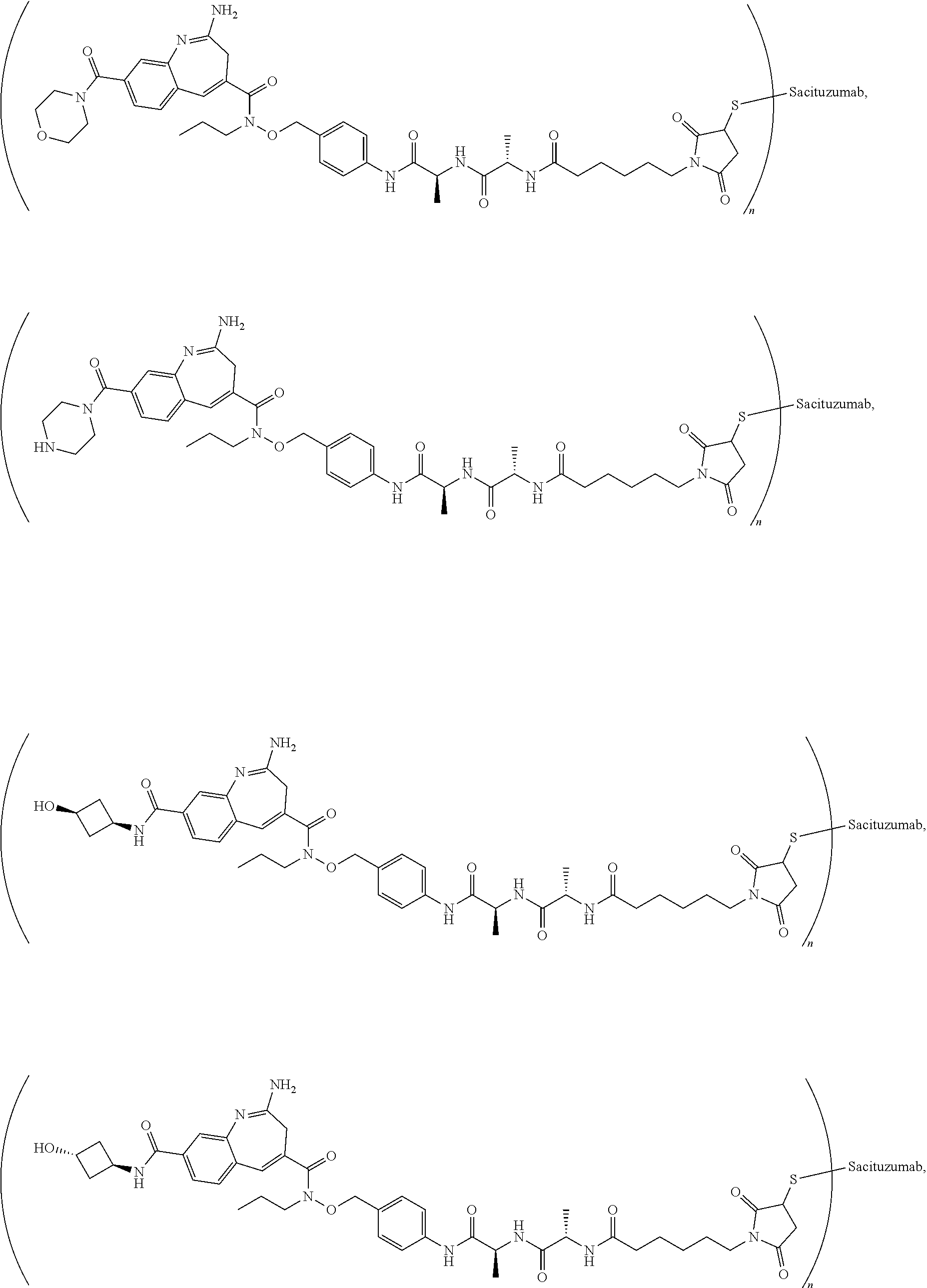

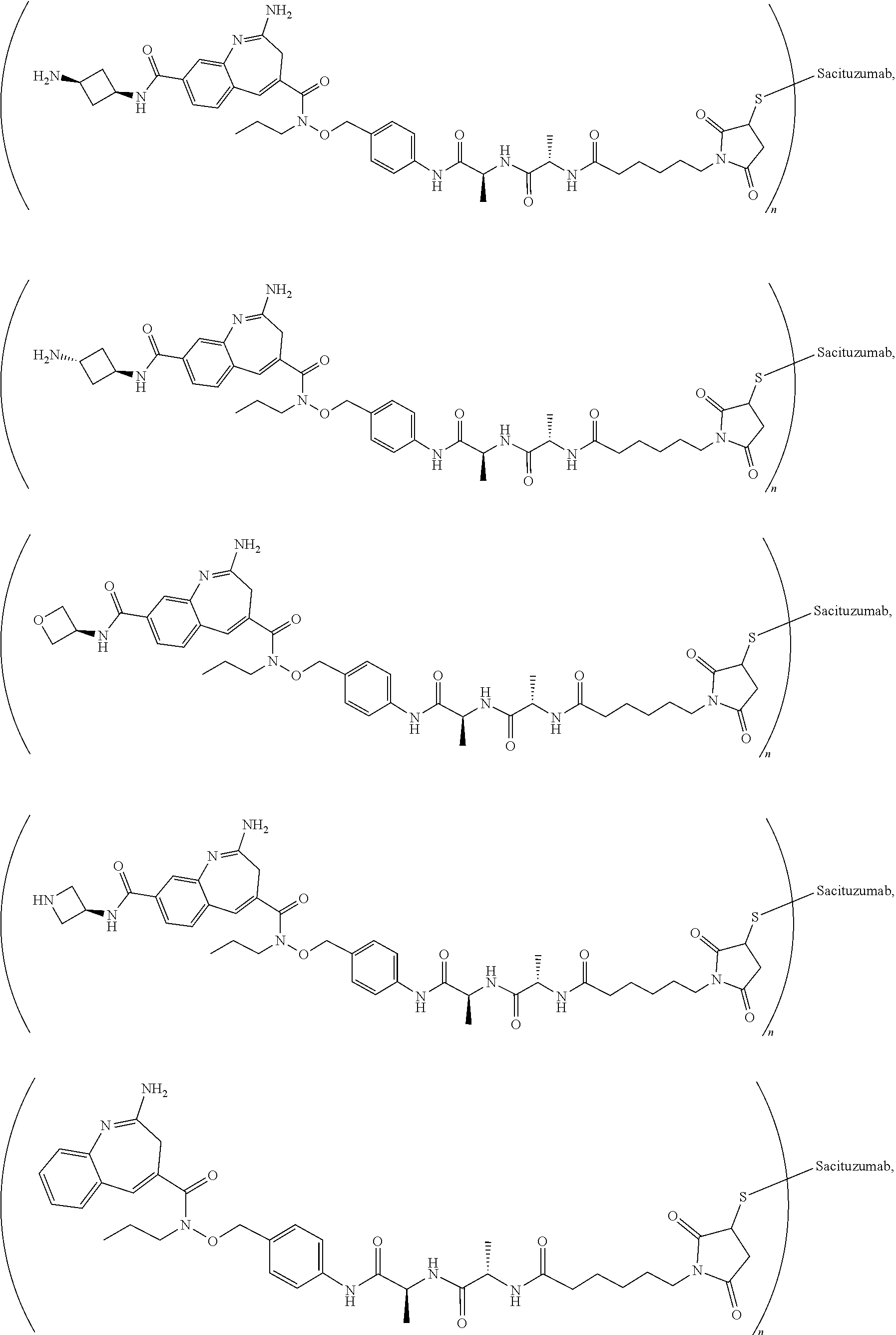

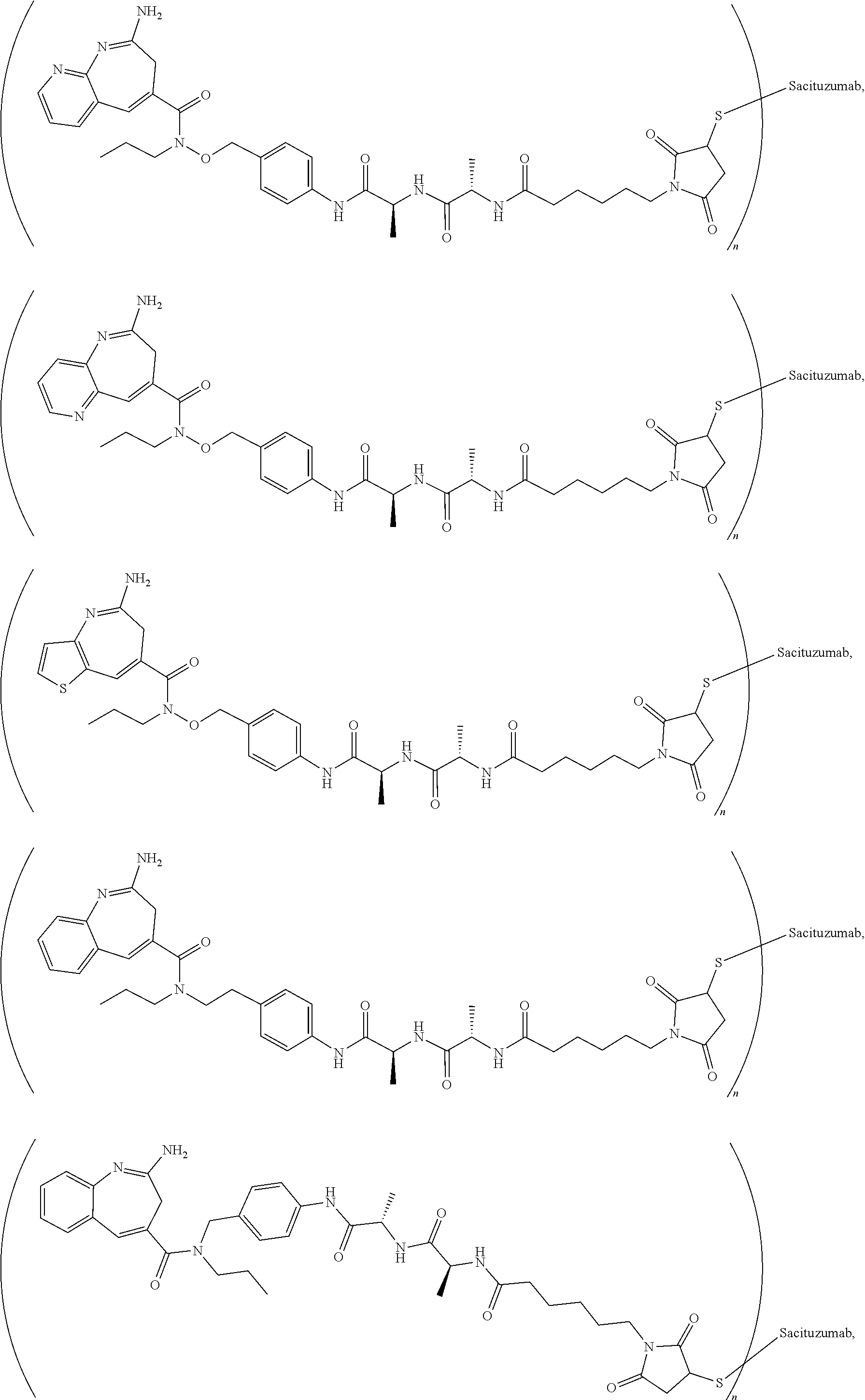

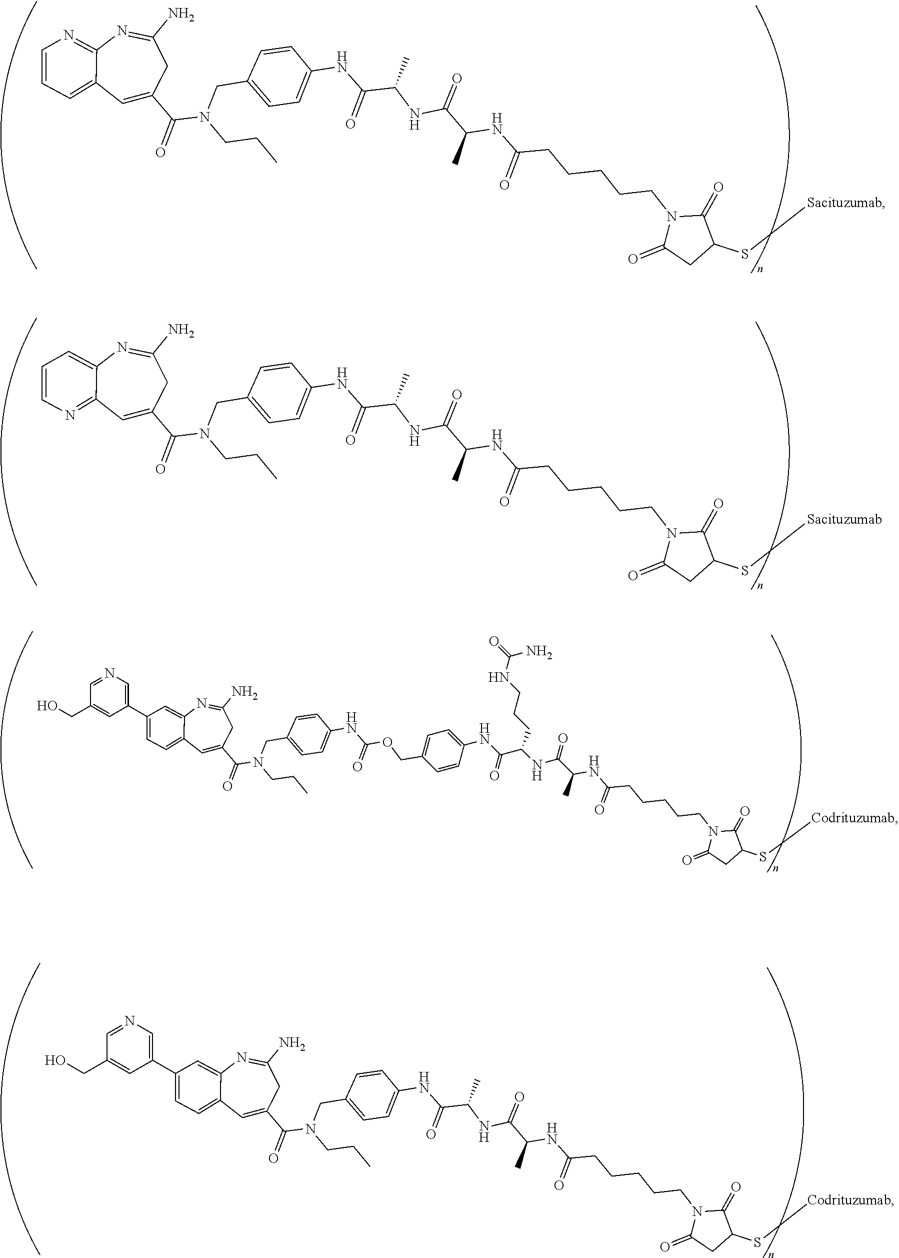

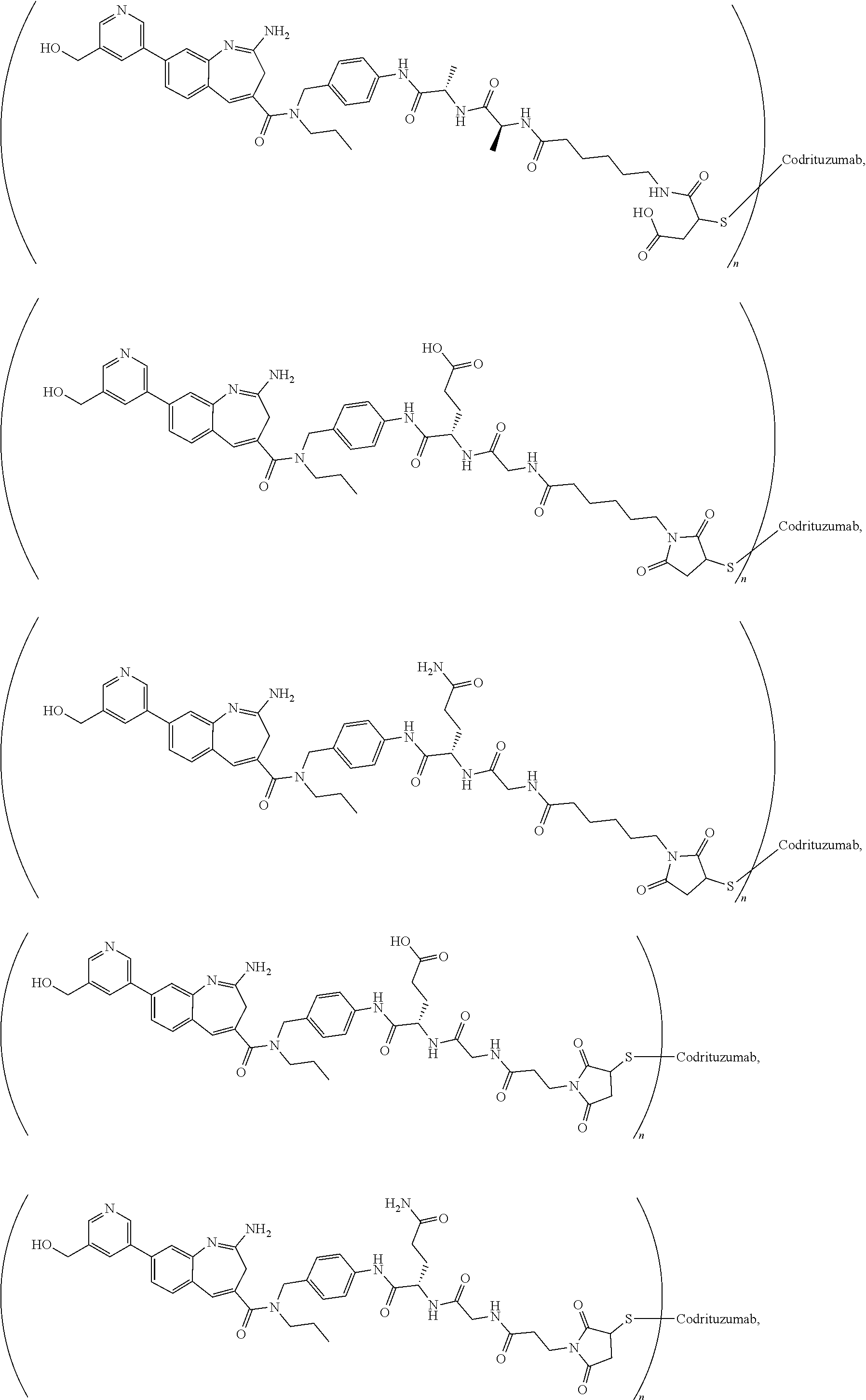

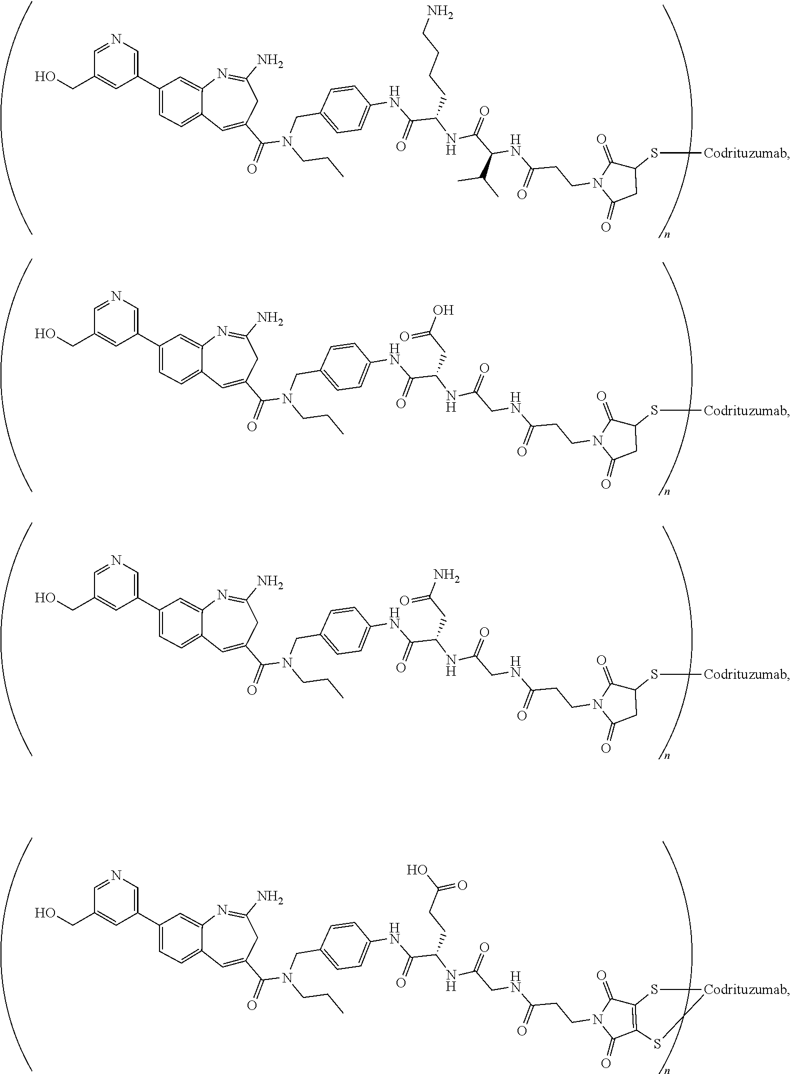

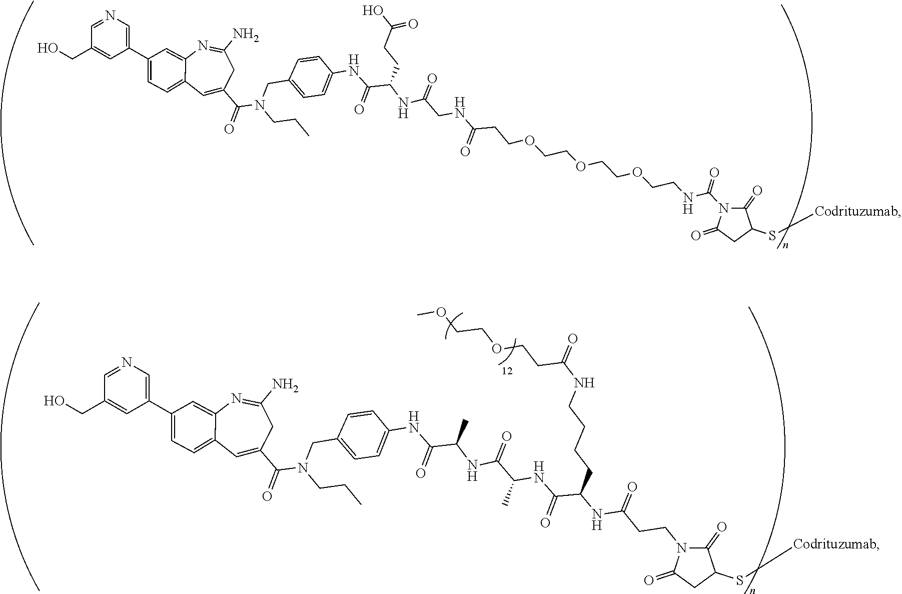

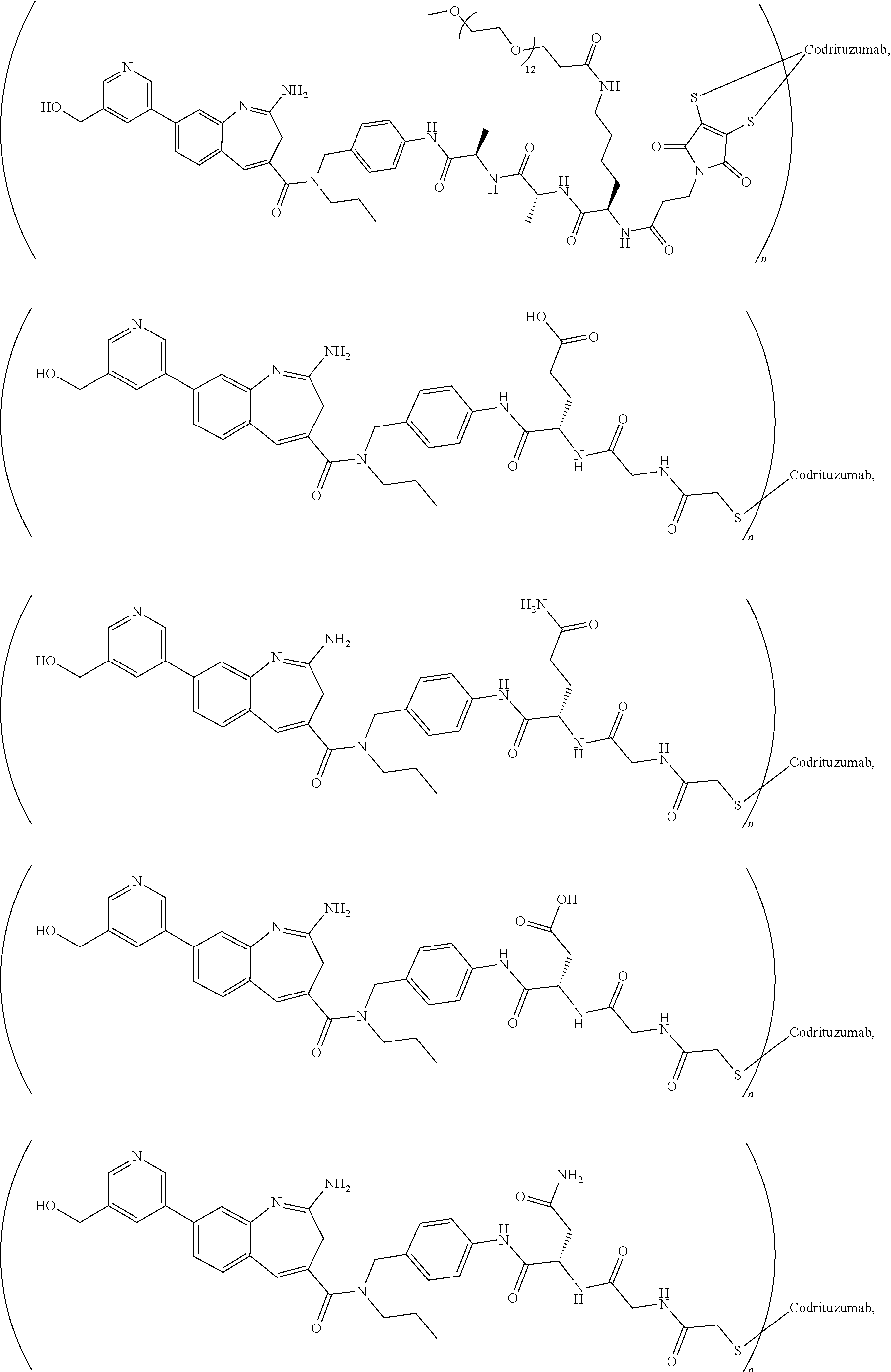

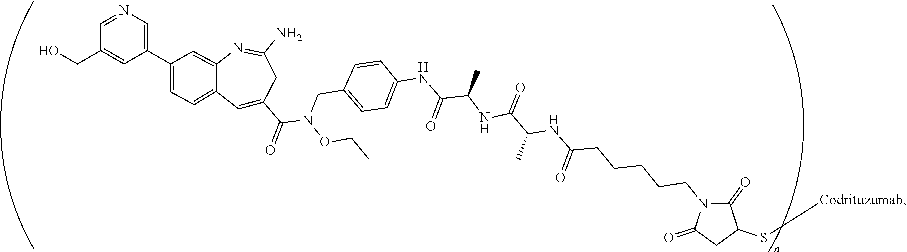

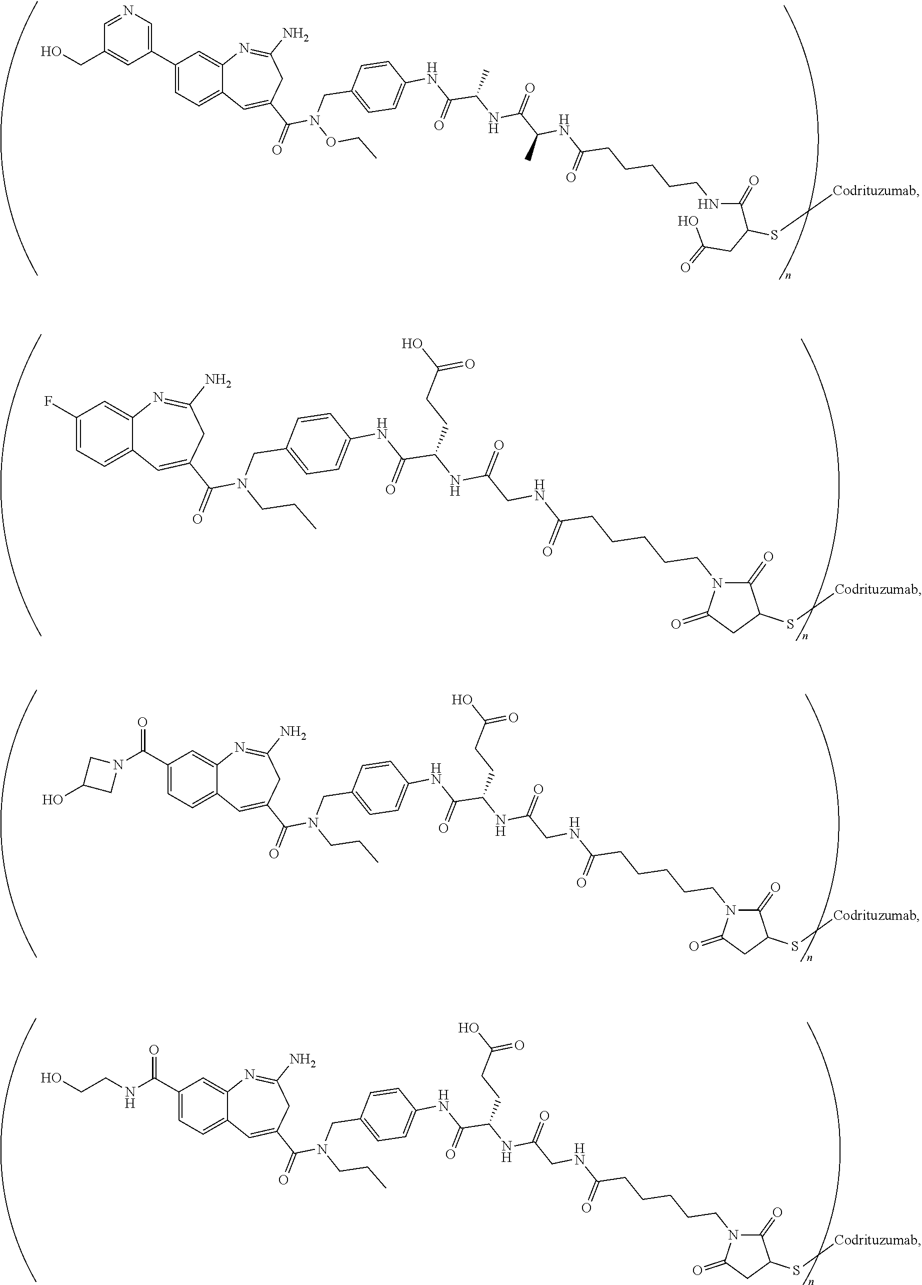

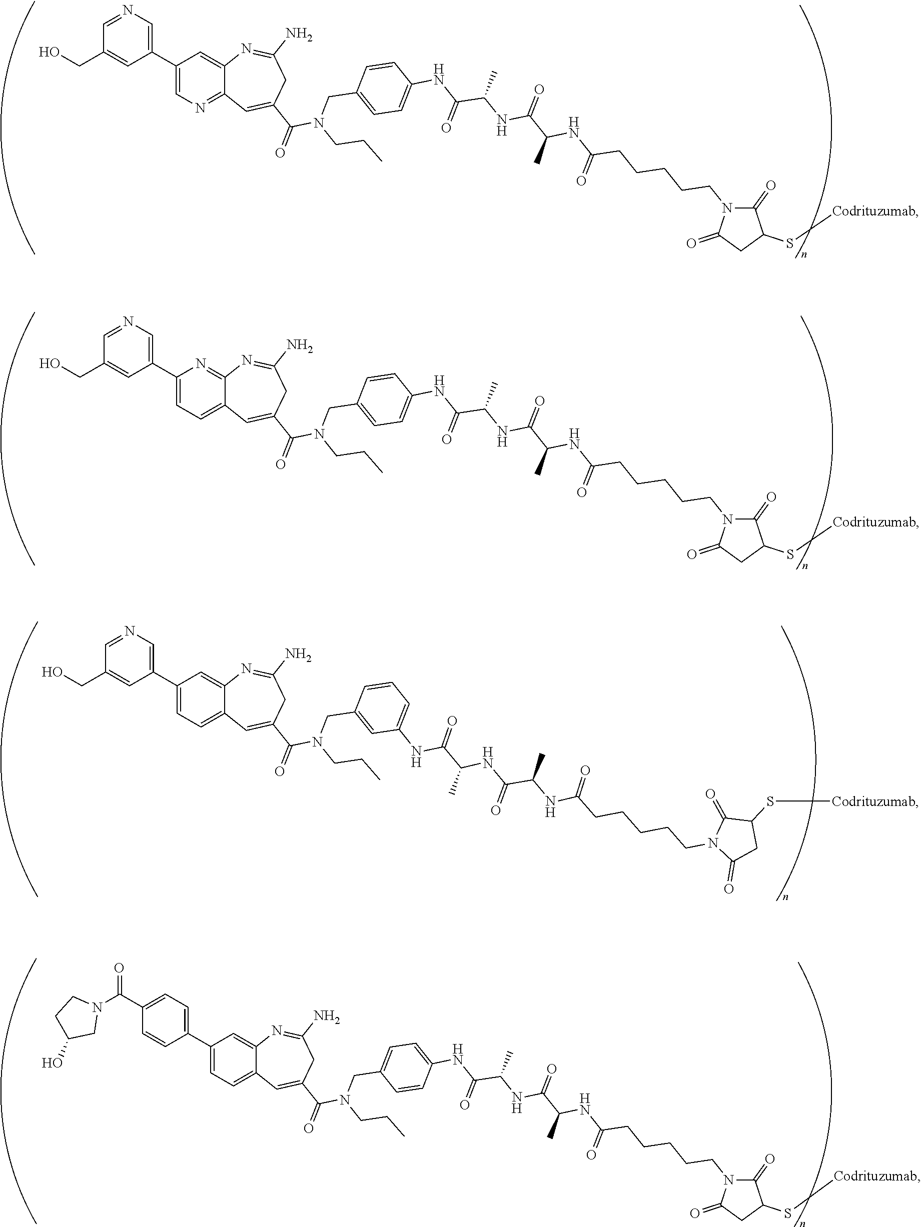

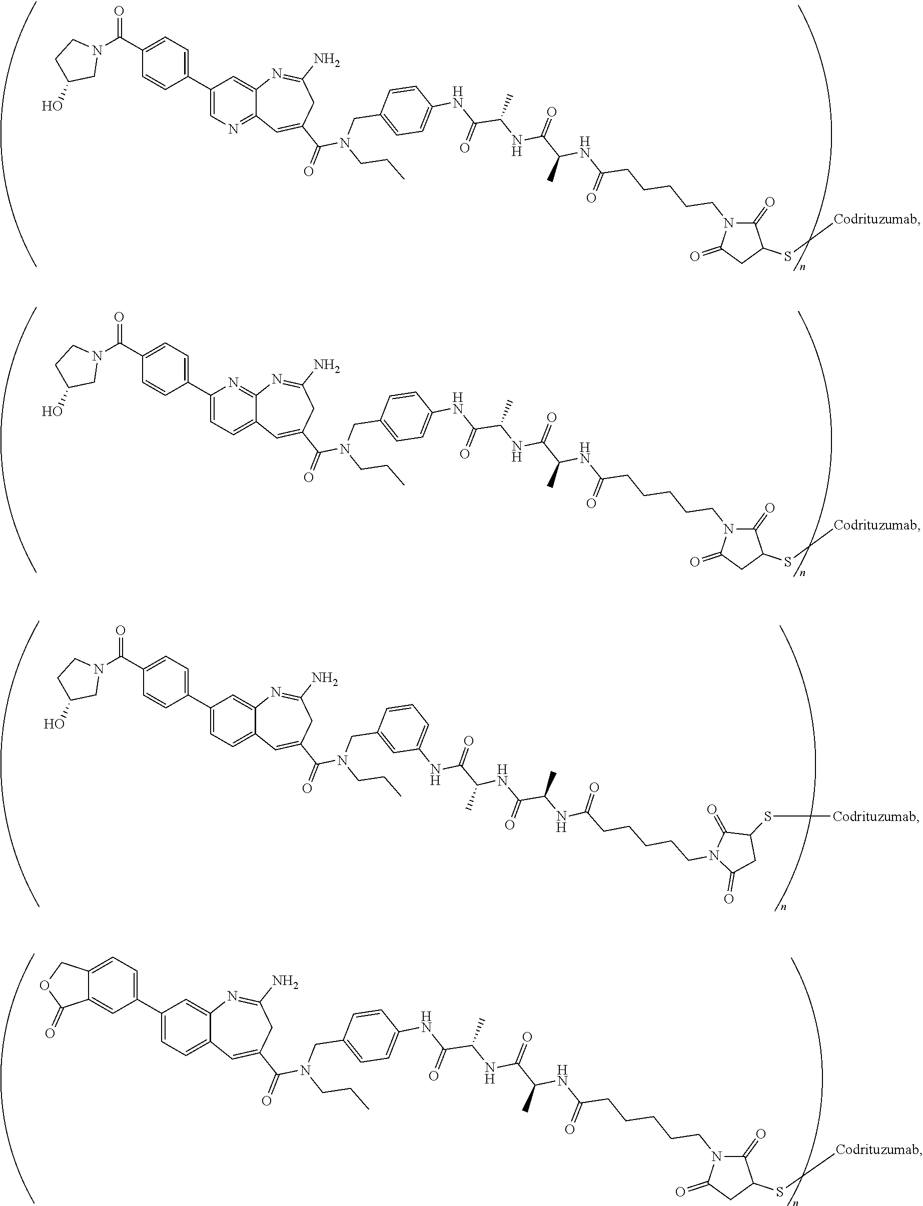

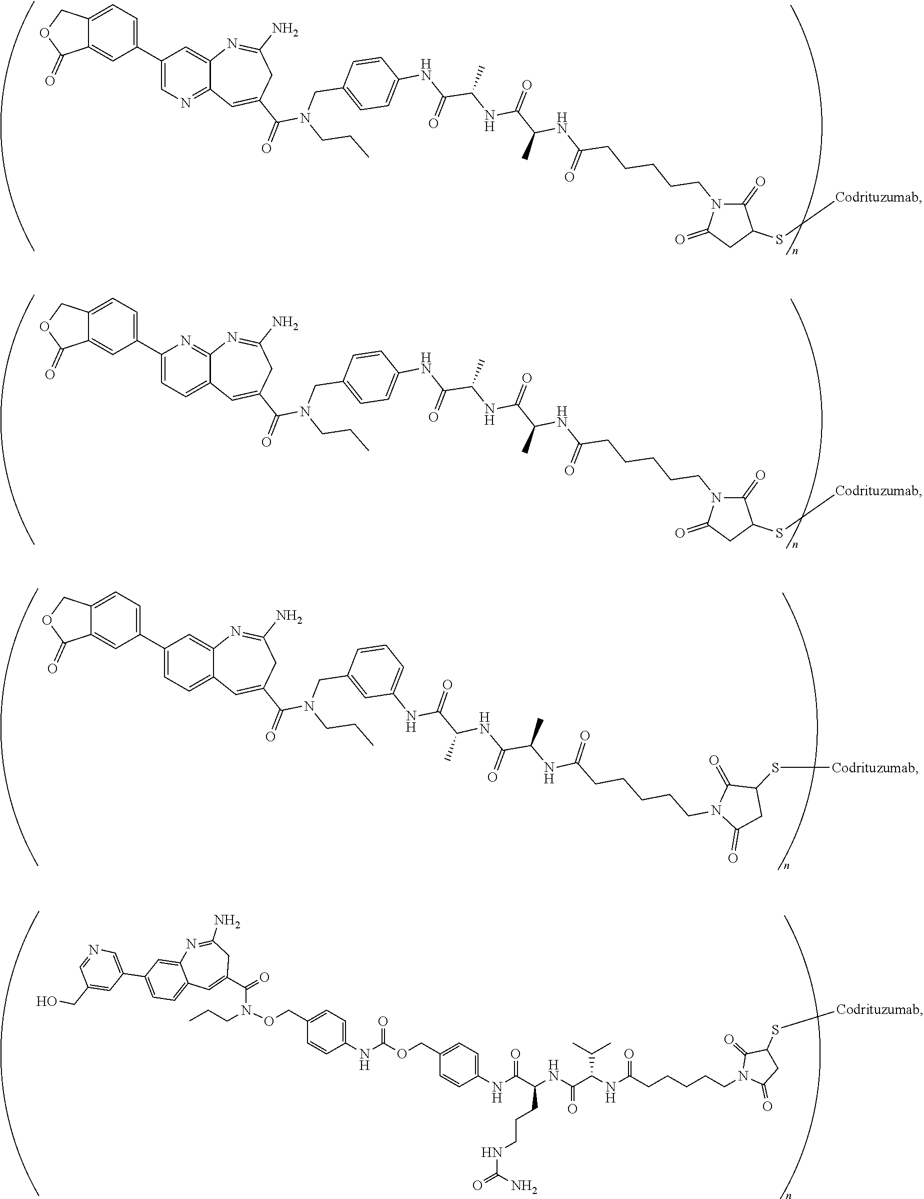

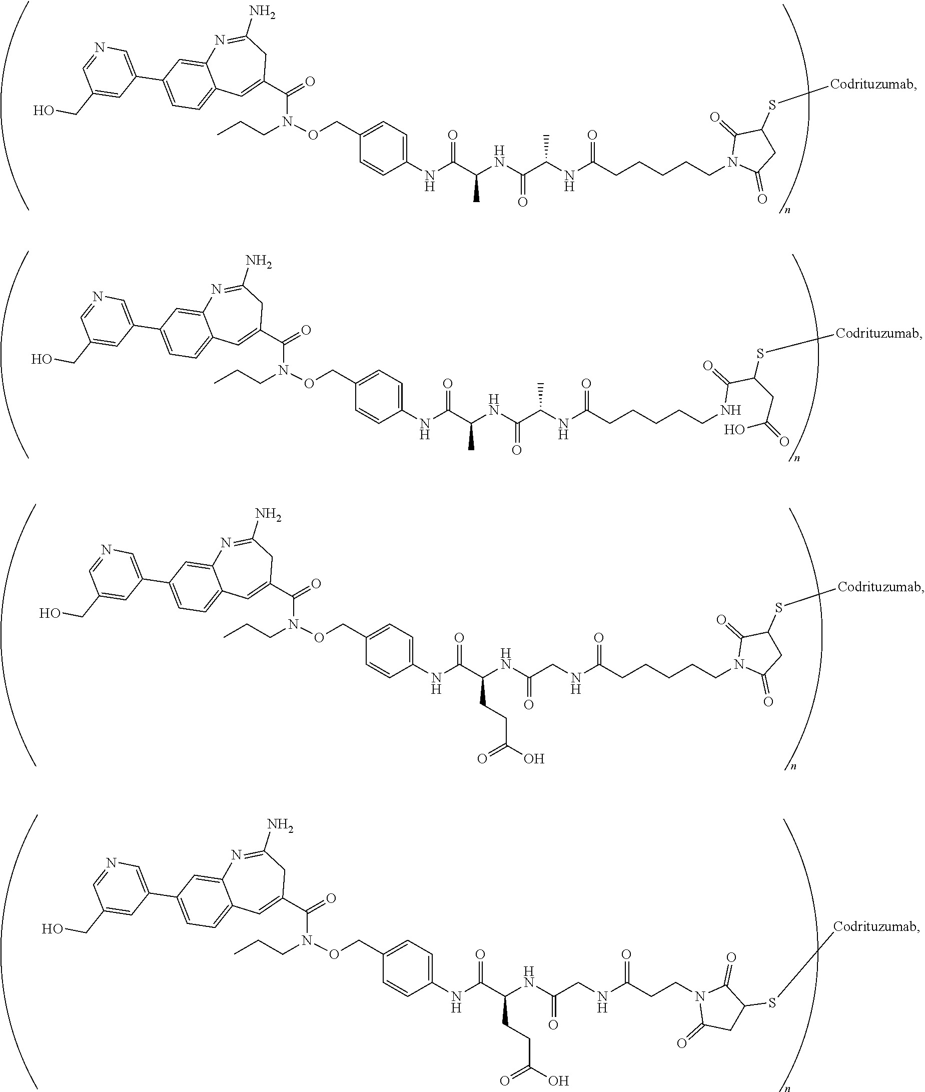

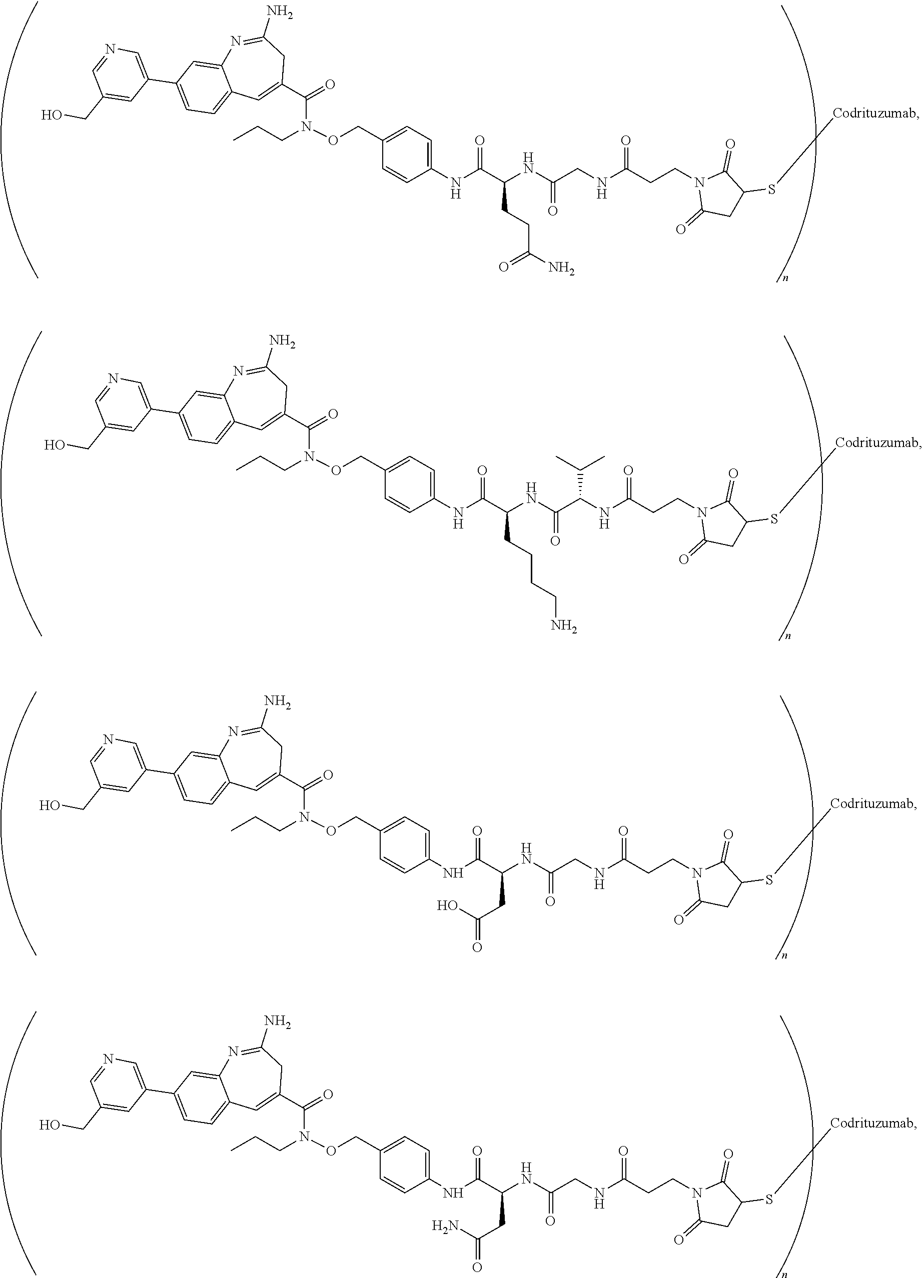

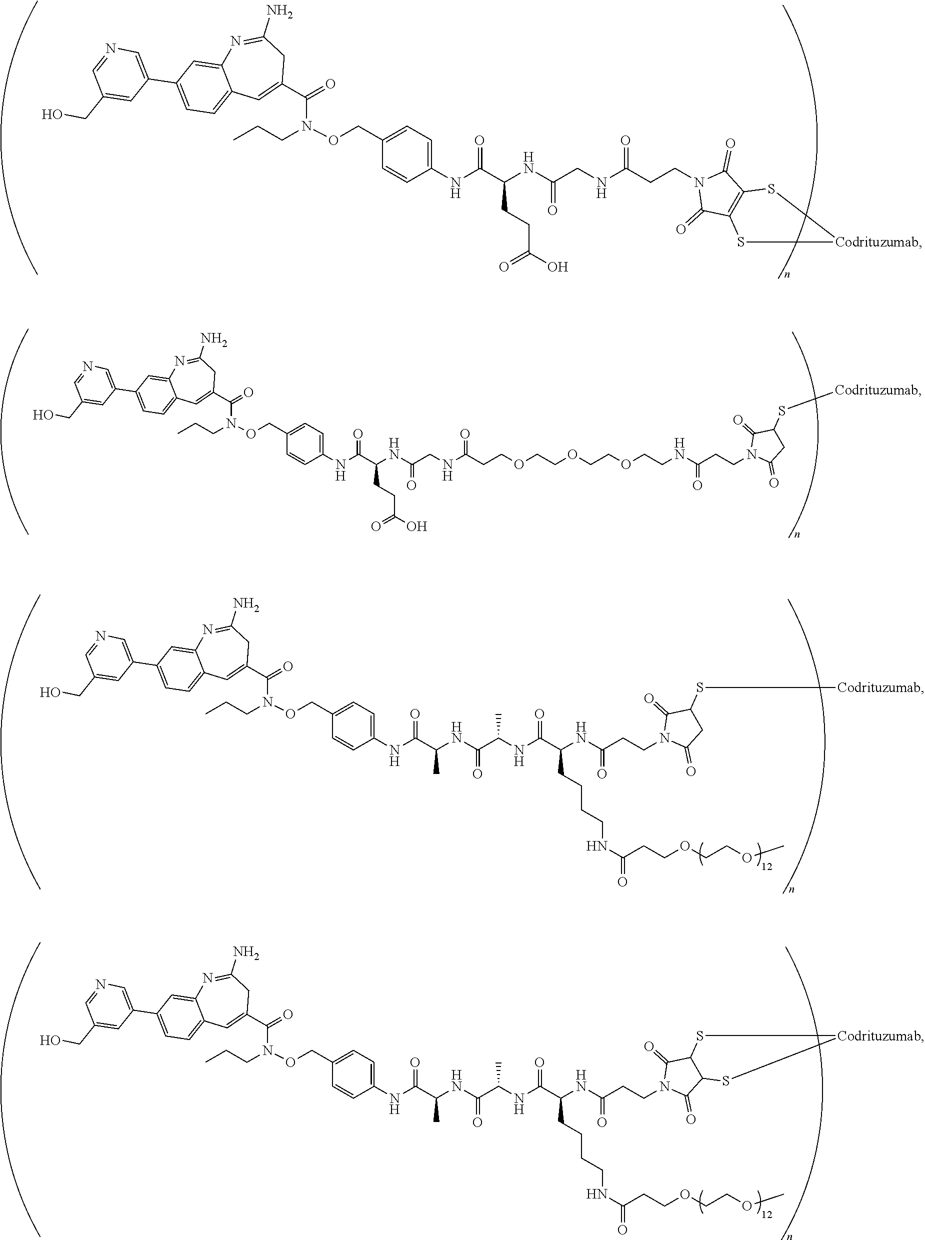

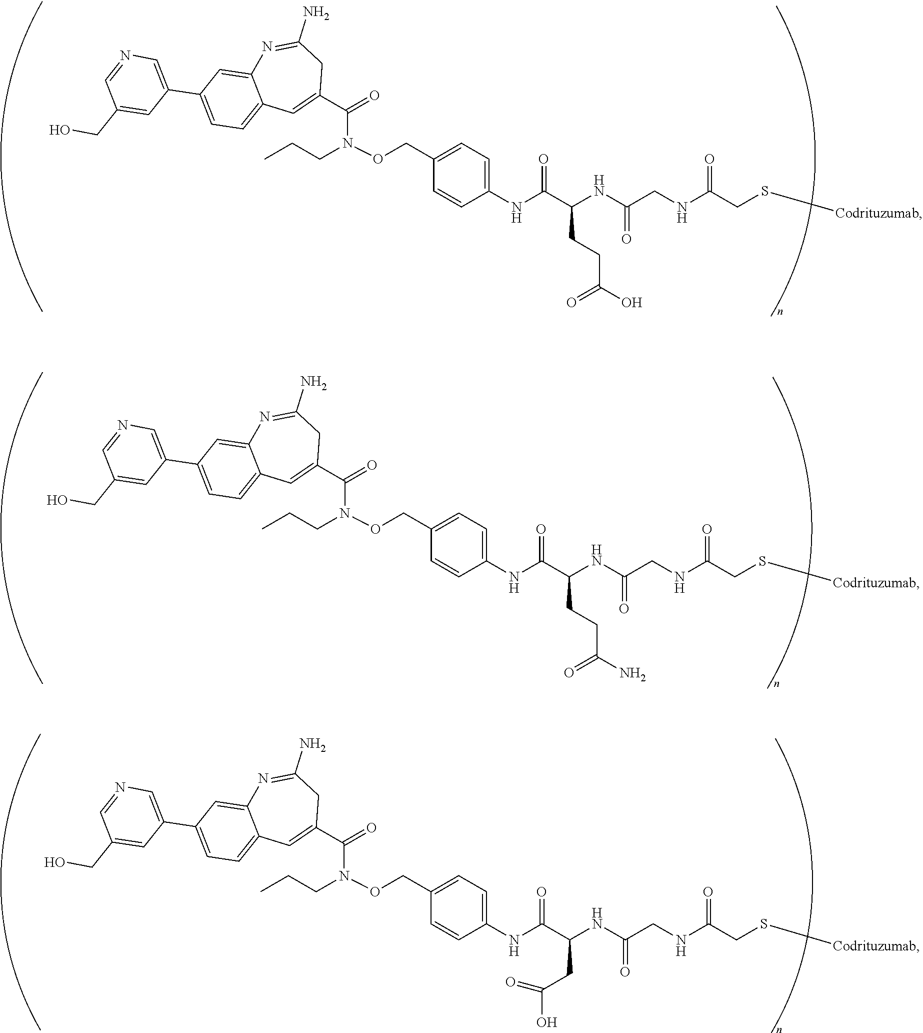

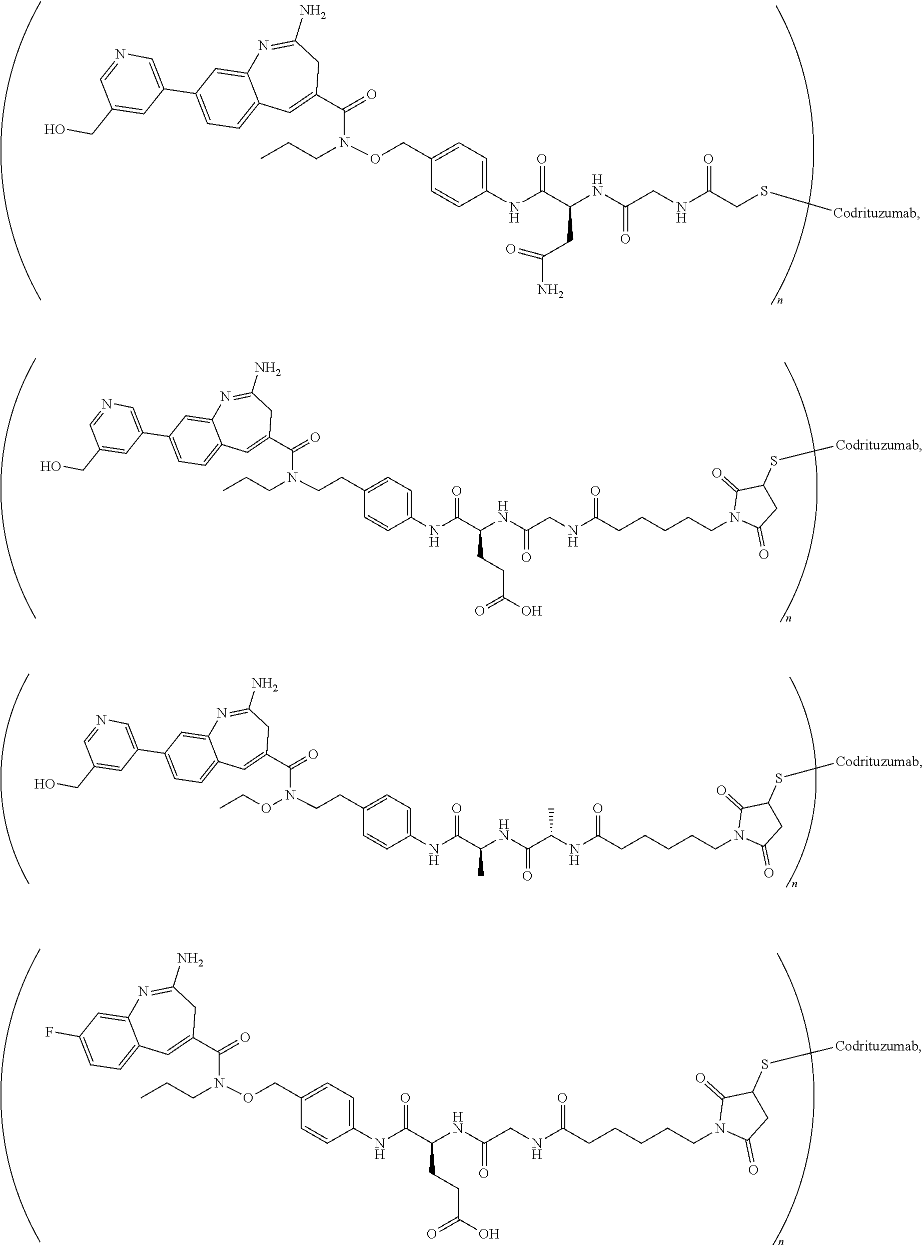

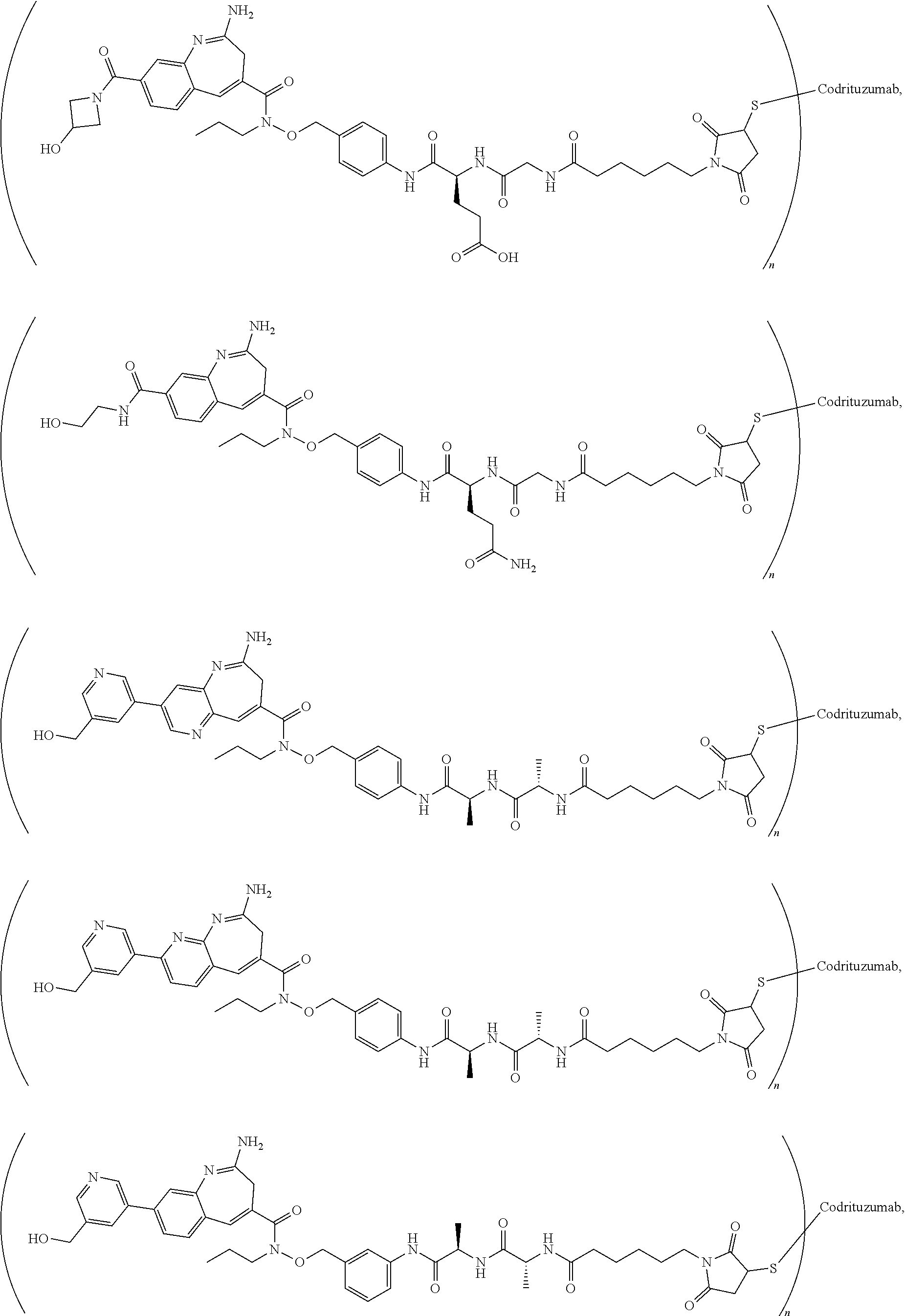

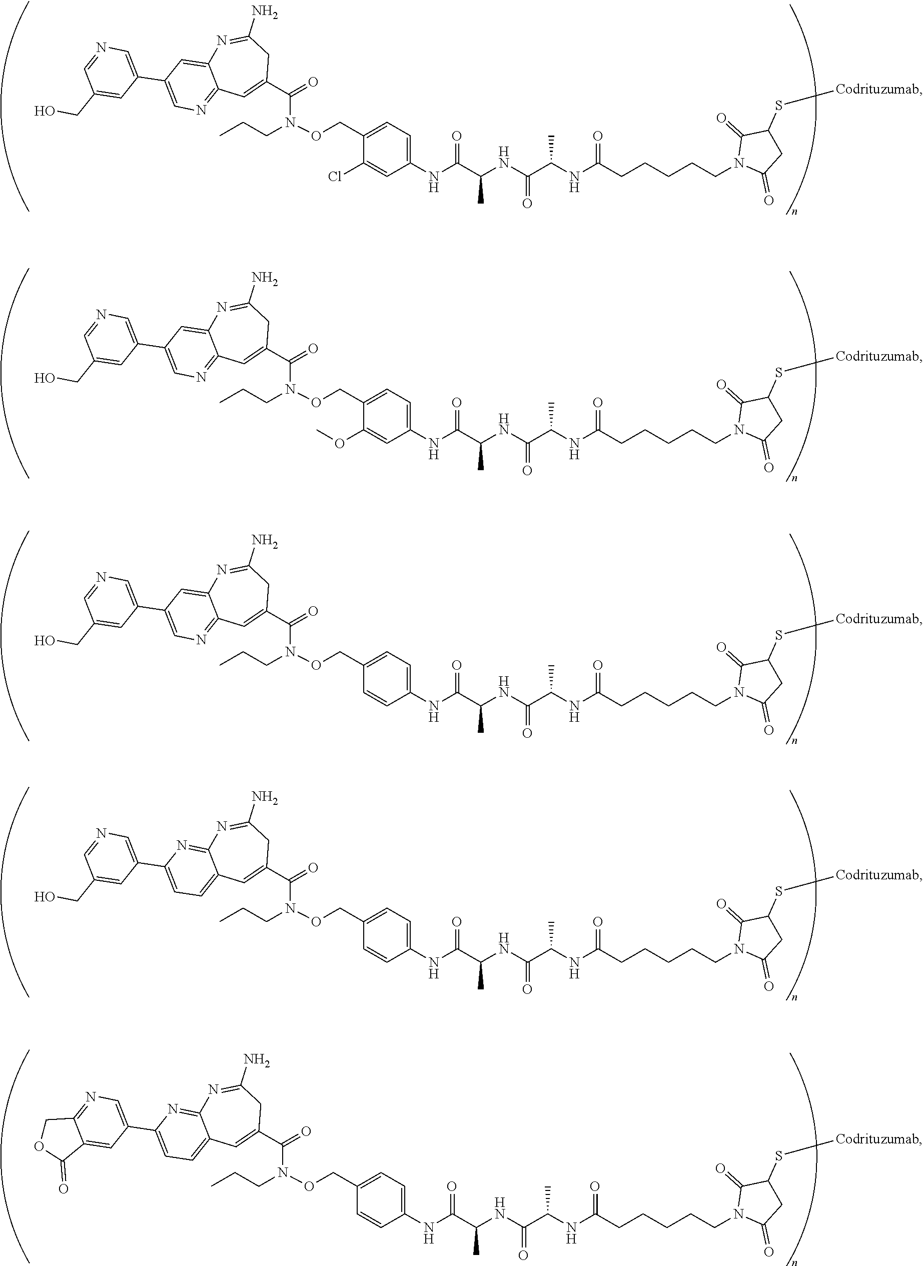

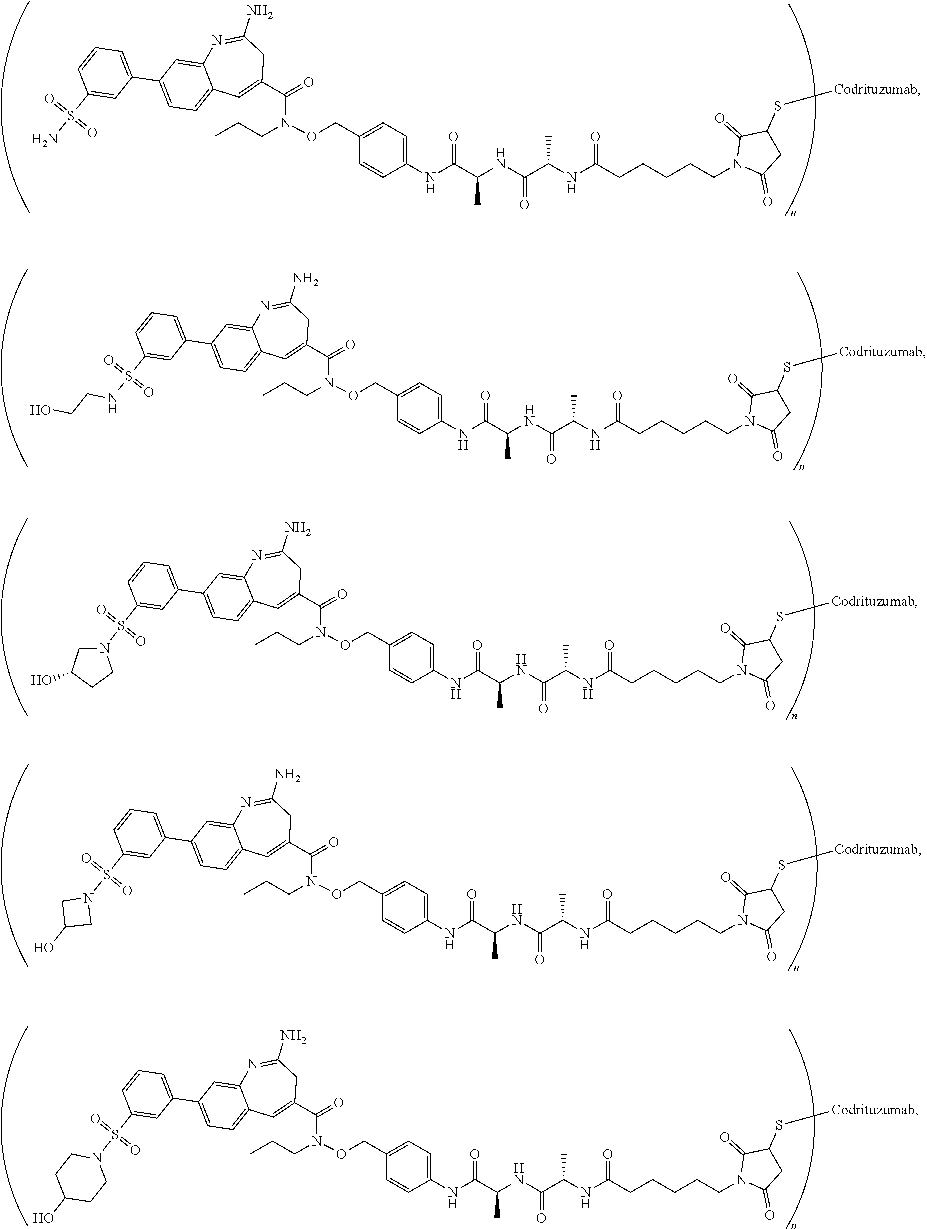

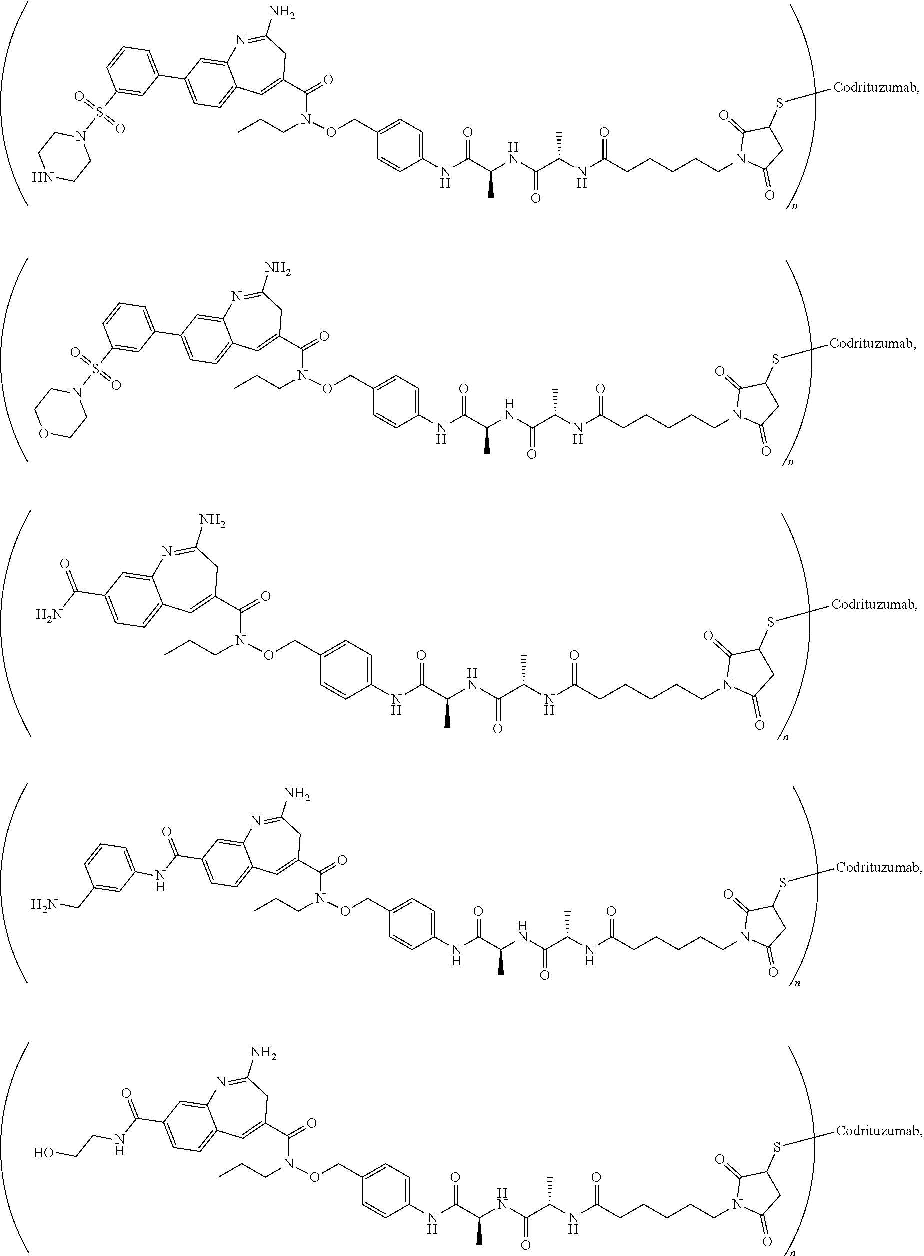

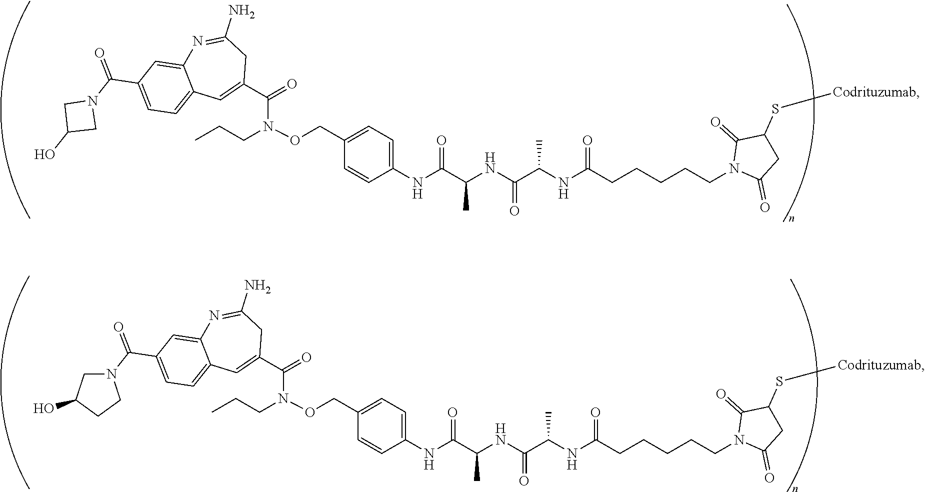

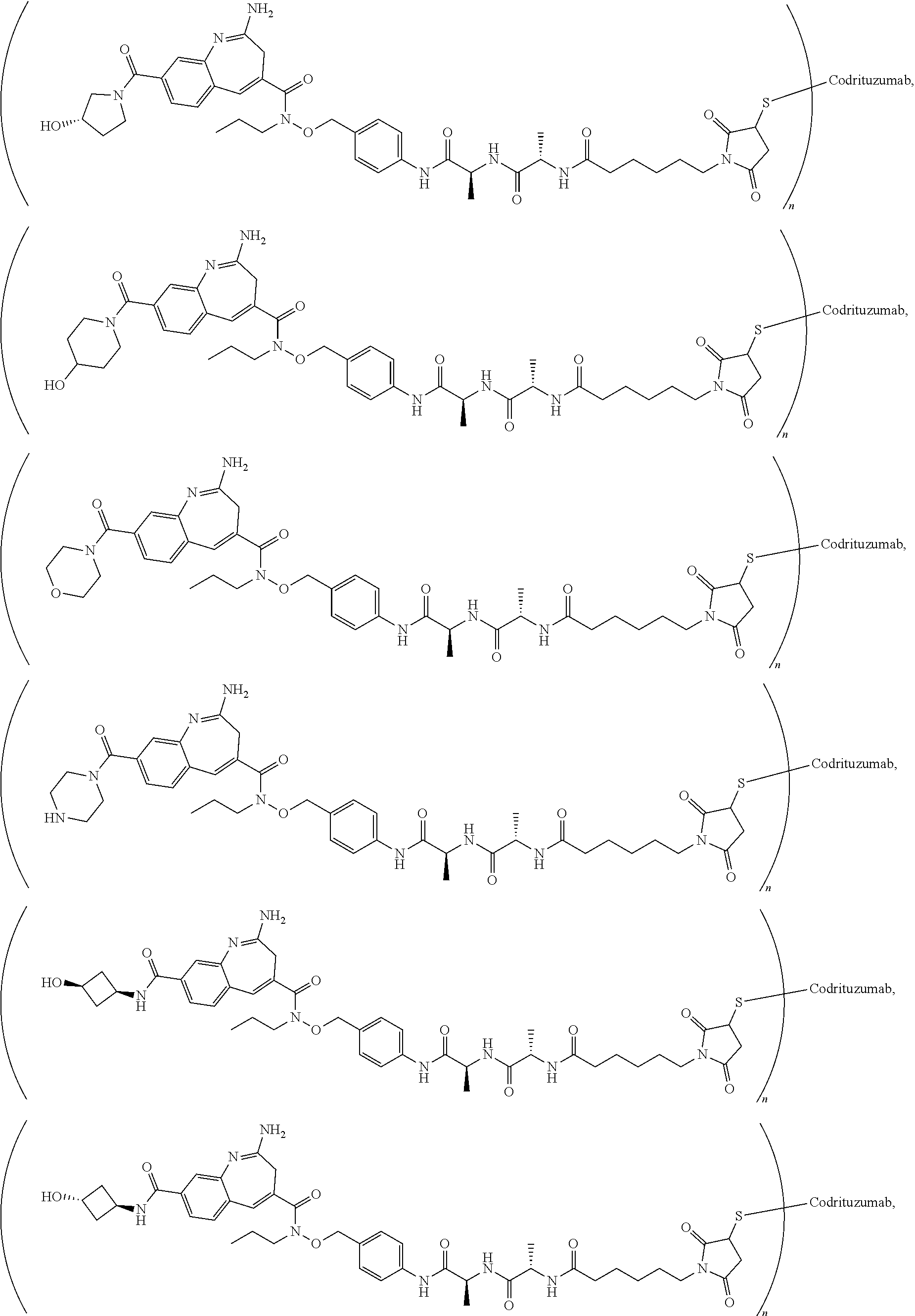

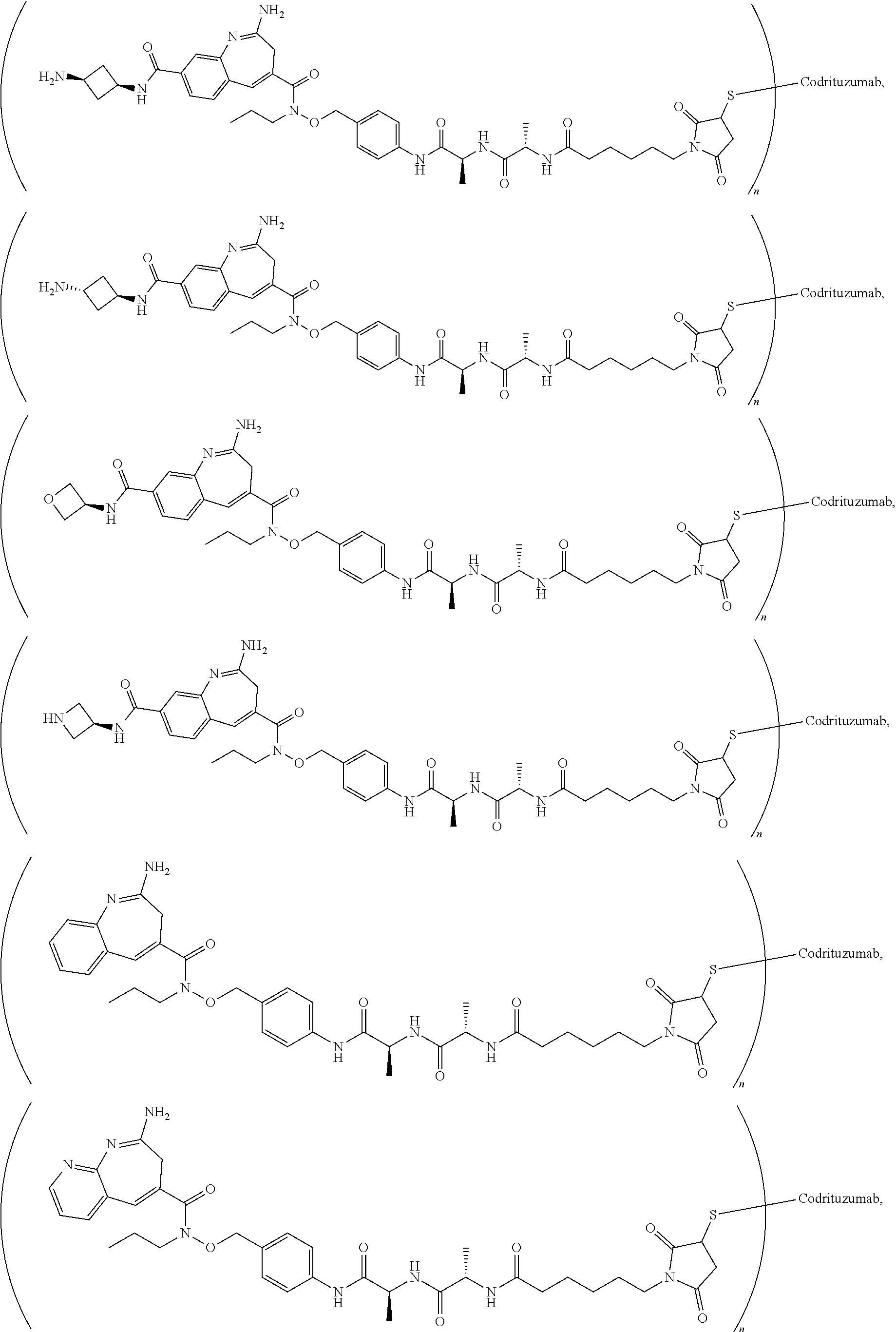

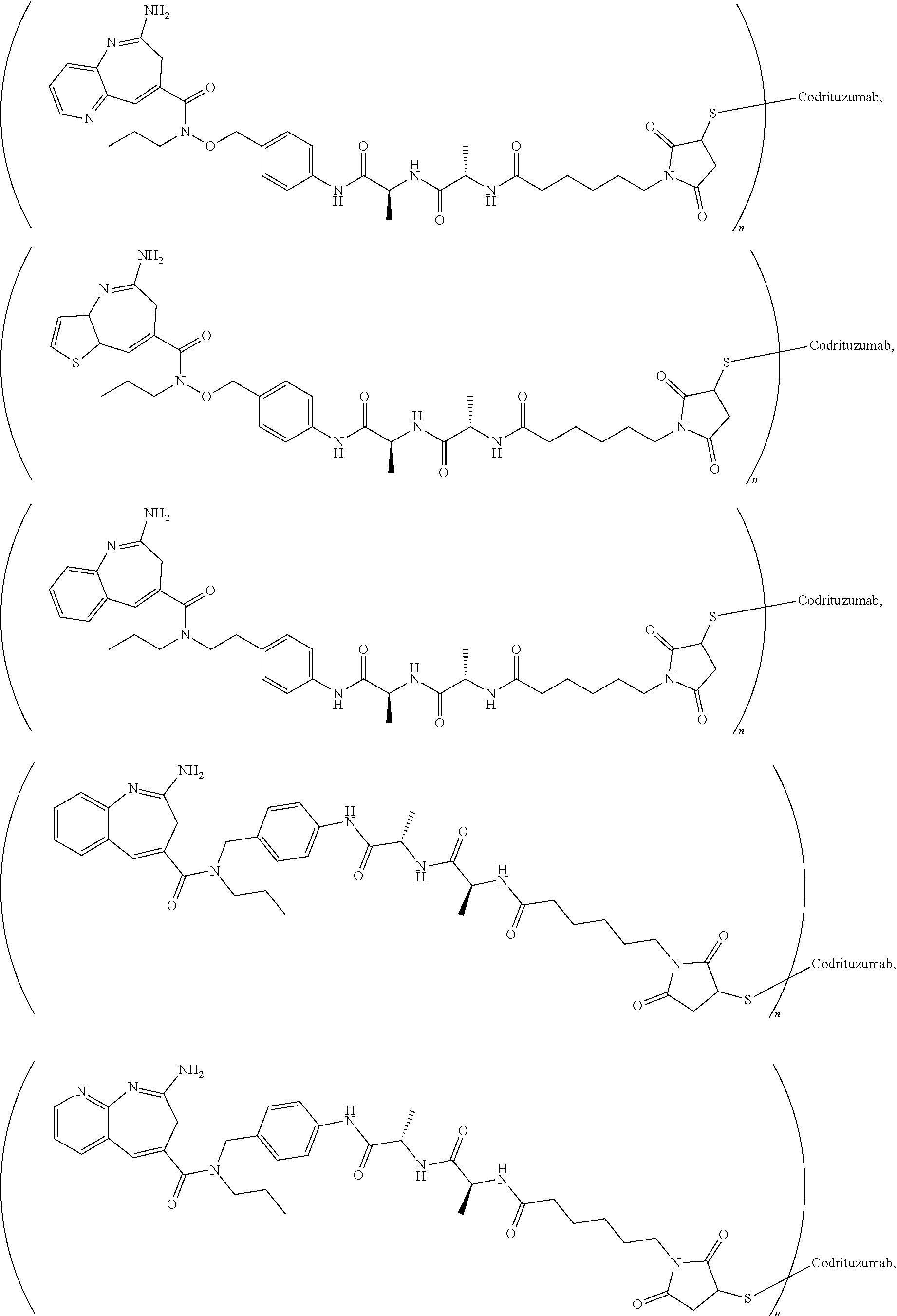

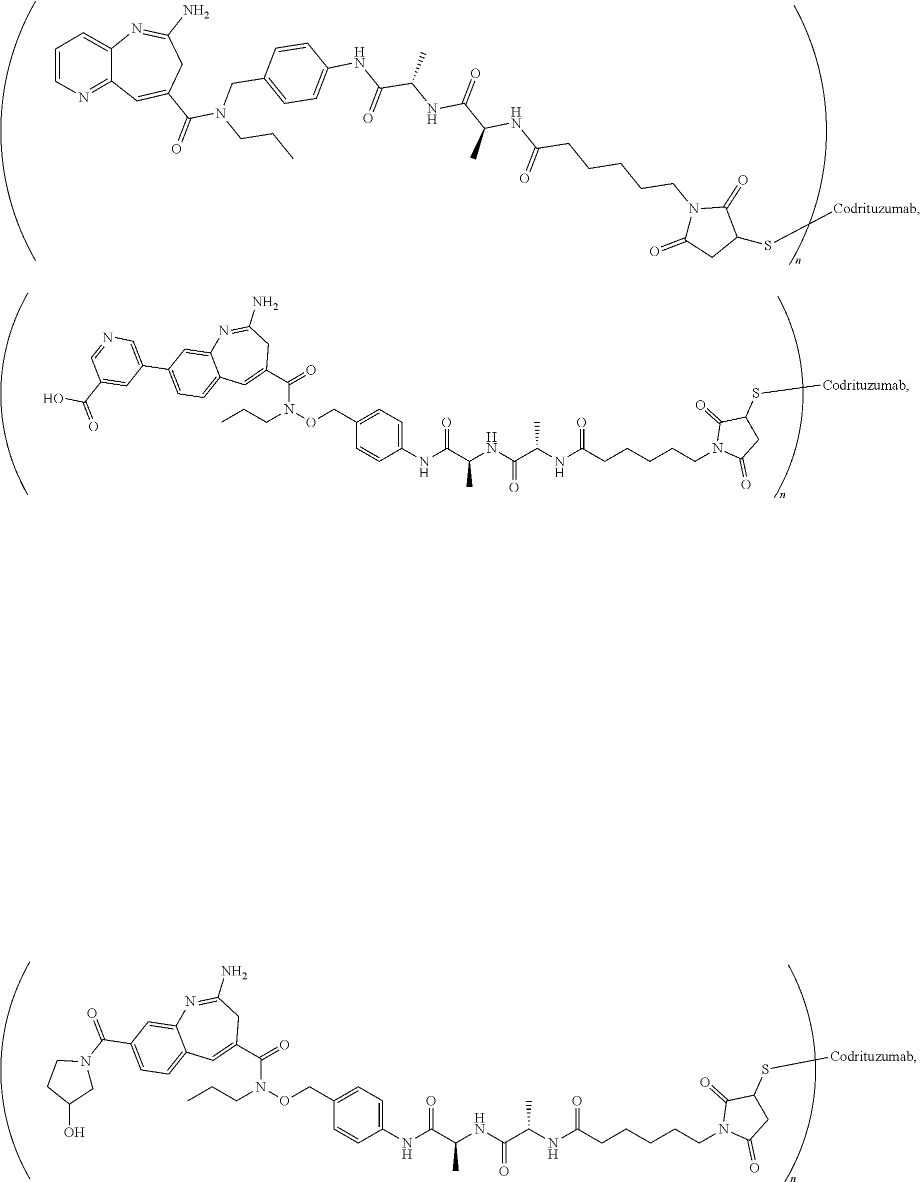

wherein
n is an integer or a decimal from 1 to 10.

In some embodiments, the compound or the conjugate thereof, or the isomer thereof, or the mixture thereof, or the pharmaceutically acceptable salt thereof of the present disclosure is selected from:

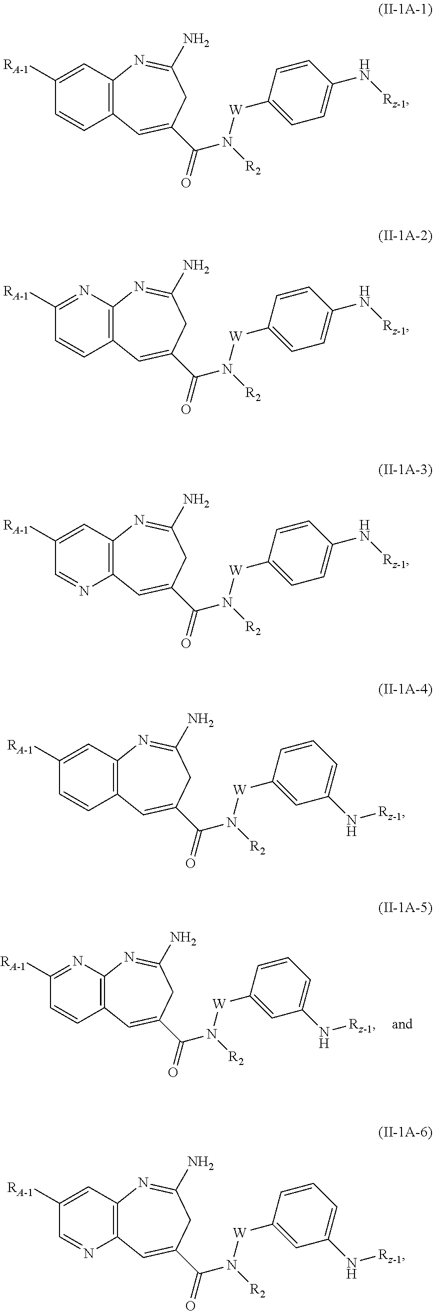

wherein
R_{Z-1} is selected from H or C₁-C₆ alkyl optionally substituted with 1, 2, or 3 fluorine, chlorine, bromine, iodine, hydroxy, or amino substituents; R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂₋₂; each R₂₋₂ is independently selected from: hydrogen, halogen, hydroxy, or amino; the methylene units of R₂ are each independently unreplaced, replaced by —O—, or replaced by —N(R₂₋₃)—; R₂₋₃ is selected from: hydrogen or C₁-C₃ alkyl;
W is selected from C₁-C₆ alkyl optionally substituted with 1, 2, or 3 R_{w-1}; each R_{w-1} is independently selected from hydrogen, halogen, hydroxy, amino, C₁-C₆ alkyl, and C₃-C₆ cycloalkyl, wherein the C₁-C₆alkyl and the C₃-C₆ cycloalkyl are each independently optionally substituted with 1, 2, or 3 R_{w-2};
each R_{w-2} is independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl;
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆alkyl), or —N(C₁-C₆ alkyl)₂.

In certain preferred embodiments of the present disclosure, in the structures represented by formulas (II-1A-1), (II-1A-2), (II-1A-3), (II-1A-4), (II-1A-5), and (II-1A-6), R_{Z-1}, R₂, W, and R_{A-1} are as described in any one of the embodiments of (II-1A) or (II-2A) of the present application.

In yet another aspect, the present disclosure provides a compound or a conjugate thereof, or an isomer thereof, or a mixture thereof, or a pharmaceutically acceptable salt thereof, wherein the compound has a structure represented by formula (II-3A):

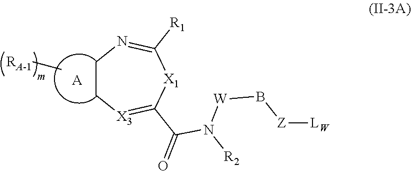

wherein
R₁ is optionally substituted NH₂;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl; ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl; when R_{A-1} comprises methylene units, the methylene units of R_{A-1} are each independently unreplaced, or are each independently replaced by any structure;
m is selected from 1, 2, or 3;
L_{w} is -L₃-L₂-L₁;
L₁ is selected from

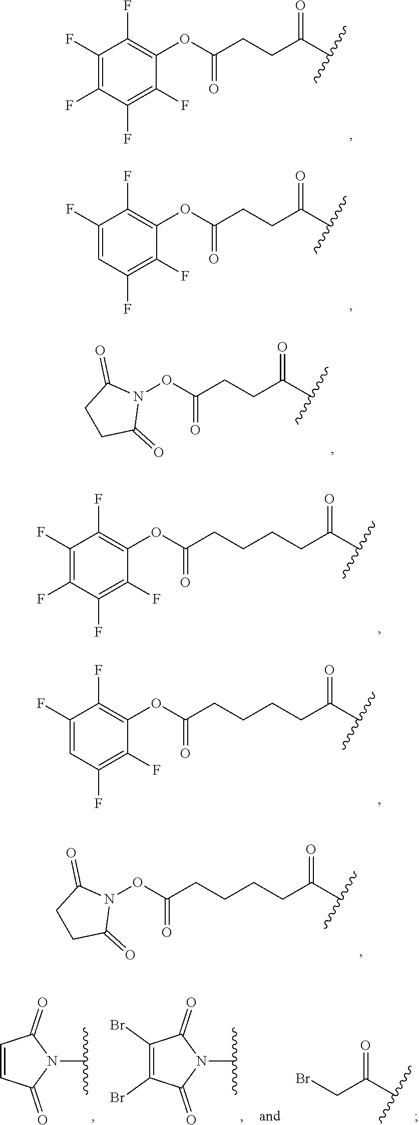

L₂ is selected from

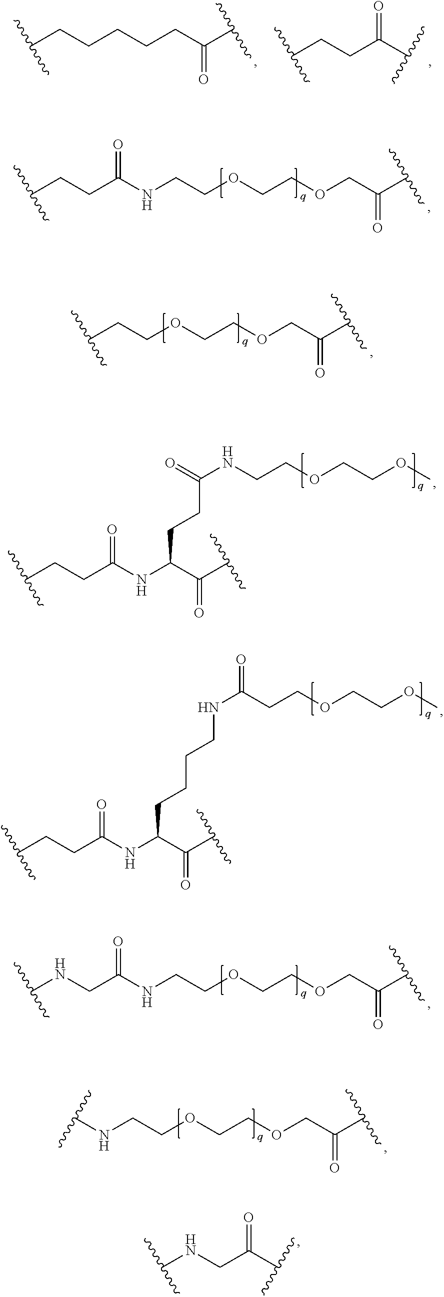

and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In some embodiments, in the structure represented by formula (II-3C), L_{w} is selected from

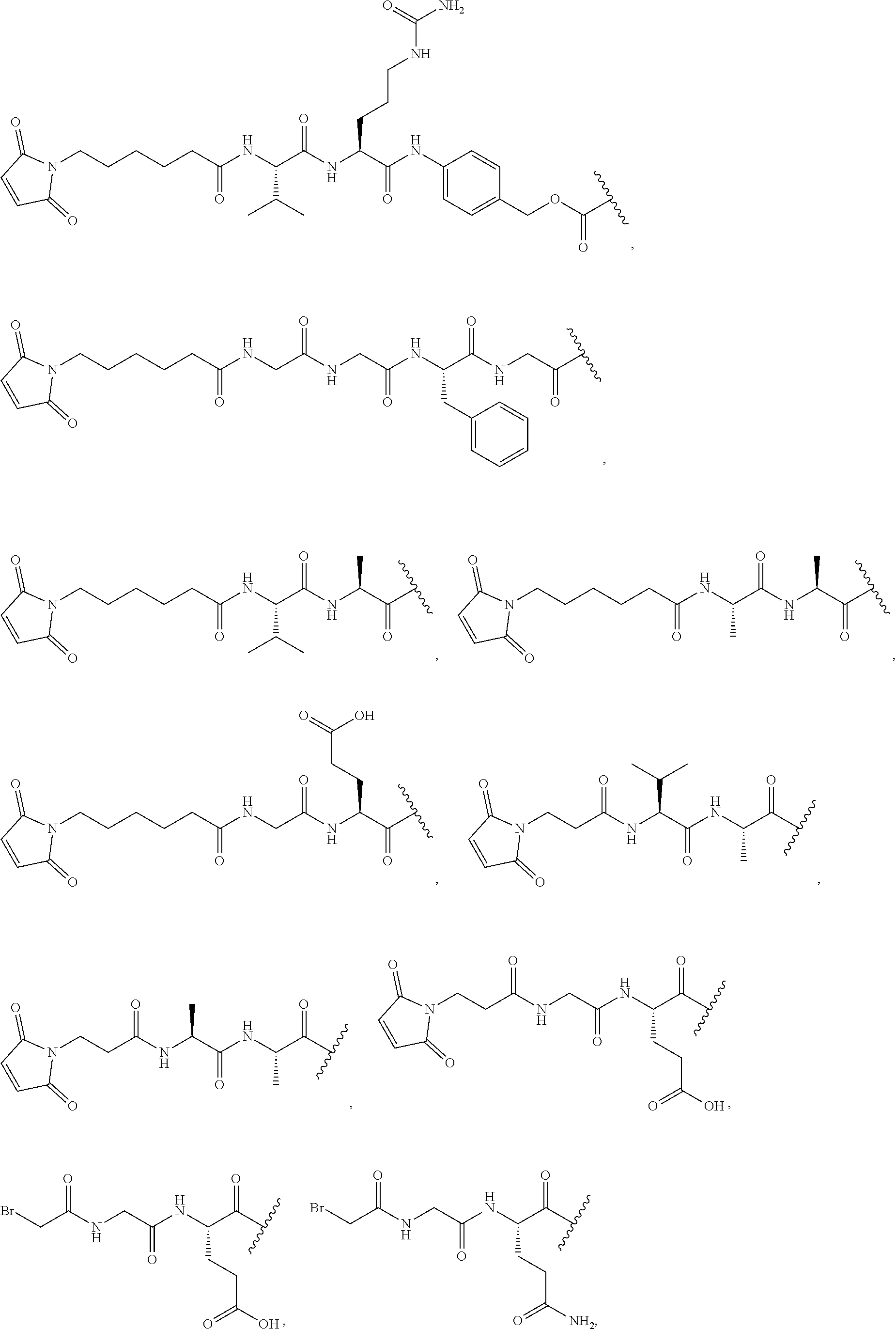

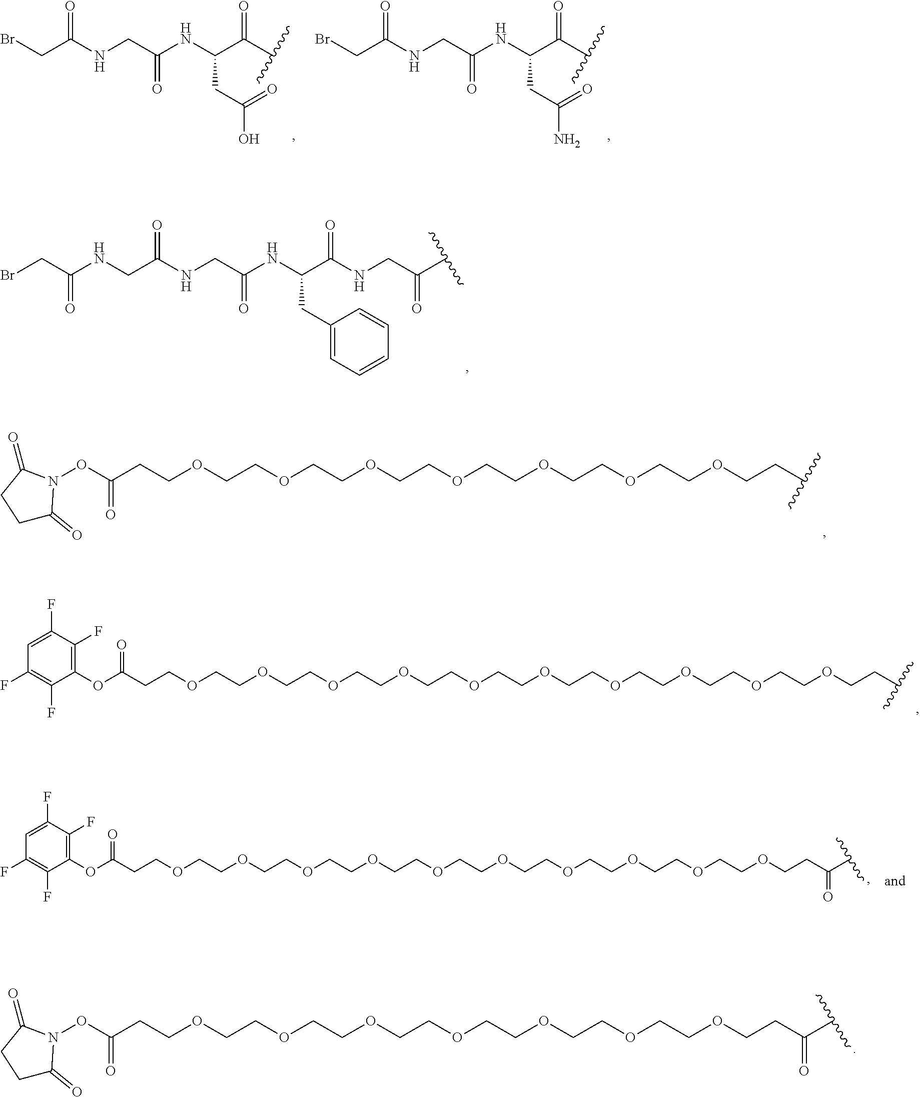

In certain preferred embodiments of the present disclosure, in the structure represented by formula (II-3A), R₁, X₁, X₃, R₂, W, B, Z, R_{A-1}, and m are as described in any one of the embodiments of (II-2B) or (II-2C) of the present application.

In some embodiments, in the compound or the conjugate thereof, or the isomer thereof, or the mixture thereof, or the pharmaceutically acceptable salt thereof of the present disclosure, the compound is represented by formulas (II-3A-1) and (II-3A-2):

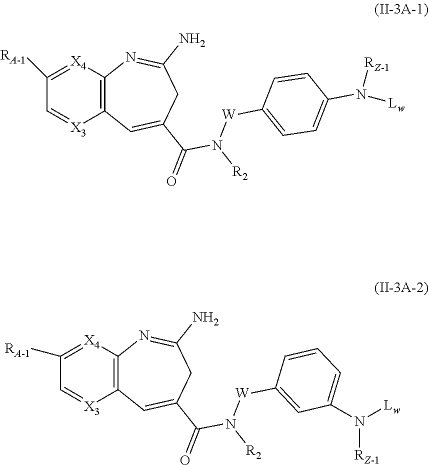

wherein
R_{Z-1} is selected from H or C₁-C₆ alkyl optionally substituted with 1, 2, or 3 fluorine, chlorine, bromine, iodine, hydroxy, or amino substituents;
X₄ and X₅ are each independently selected from —CH═ and —N═;
R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂₋₂; each R₂₋₂ is independently selected from: hydrogen, halogen, hydroxy, or amino; the methylene units of R₂ are each independently unreplaced, replaced by —O—, or replaced by —N(R₂₋₃)—; R₂₋₃ is selected from: hydrogen or C₁-C₃ alkyl;
W is selected from C₁-C₆ alkyl optionally substituted with 1, 2, or 3 R_{w-1}; each R_{w-1} is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2}; each R_{w-2} is independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl;
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein
each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆ alkyl), or —N(C₁-C₆ alkyl)₂;
L_{w} is selected from

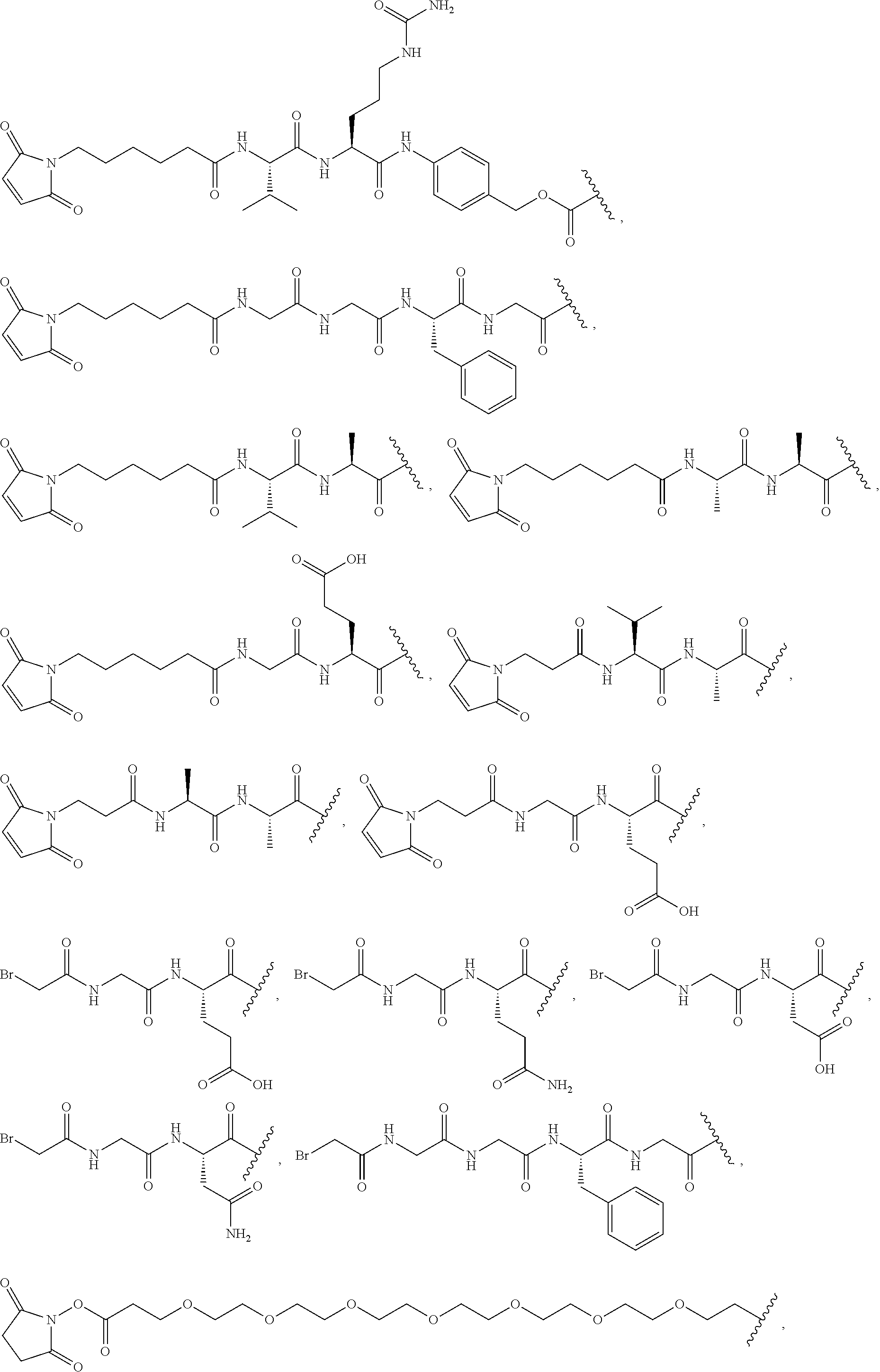

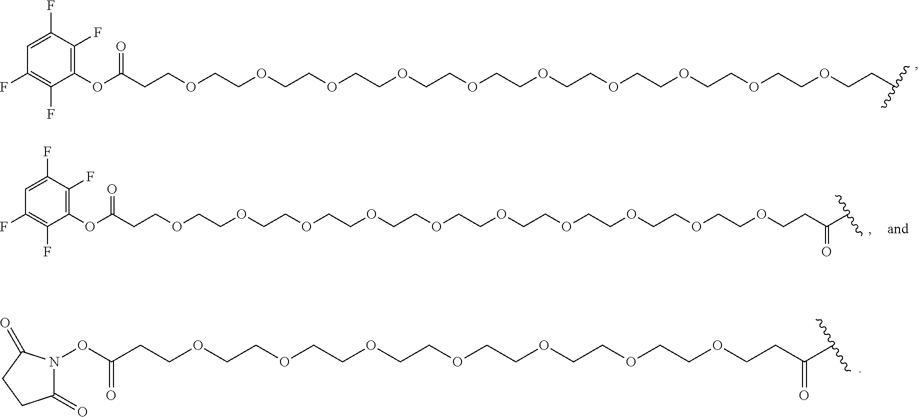

In certain preferred embodiments of the present disclosure, in the structures represented by formulas (II-3A-1) and (II-3A-2), X₄ and X₅
are each independently selected from —CH═ and —N═; R₂, W, R_{Z-1}, and R_{A-1} are as described in any one of the embodiments of (II-2B) or (II-2C) of the present application.

In yet another aspect, the present disclosure provides a compound or a conjugate thereof, or an isomer thereof, or a mixture thereof, or a pharmaceutically acceptable salt thereof, selected from:

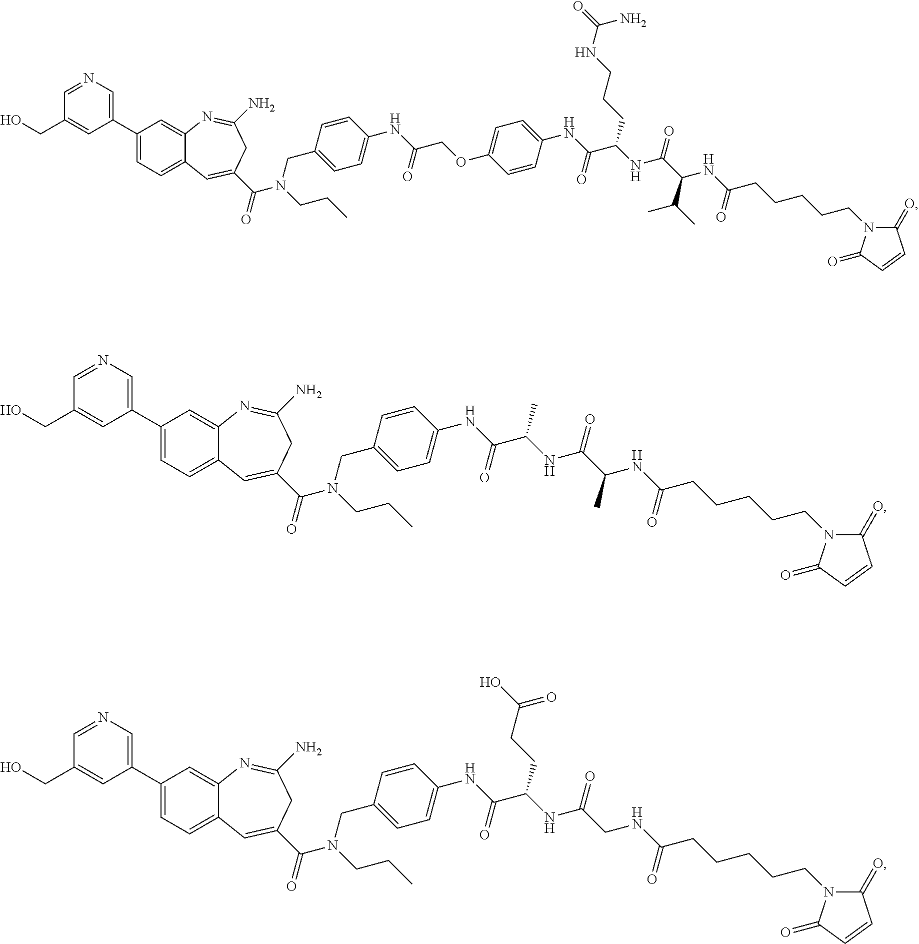 [SMILES: CCCN(Cc1ccc(NC(=O)COc2ccc(NC(=O)[C@H](CCCNC(N)=O)NC(=O)[C@@H](NC(=O)CCCCCN3C(=O)C=CC3=O)C(C)C)cc2)cc1)C(=O)C1=Cc2ccc(-c3cncc(CO)c3)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCCCCN2C(=O)C=CC2=O)cc1)C(=O)C1=Cc2ccc(-c3cncc(CO)c3)cc2N=C(N)C1]

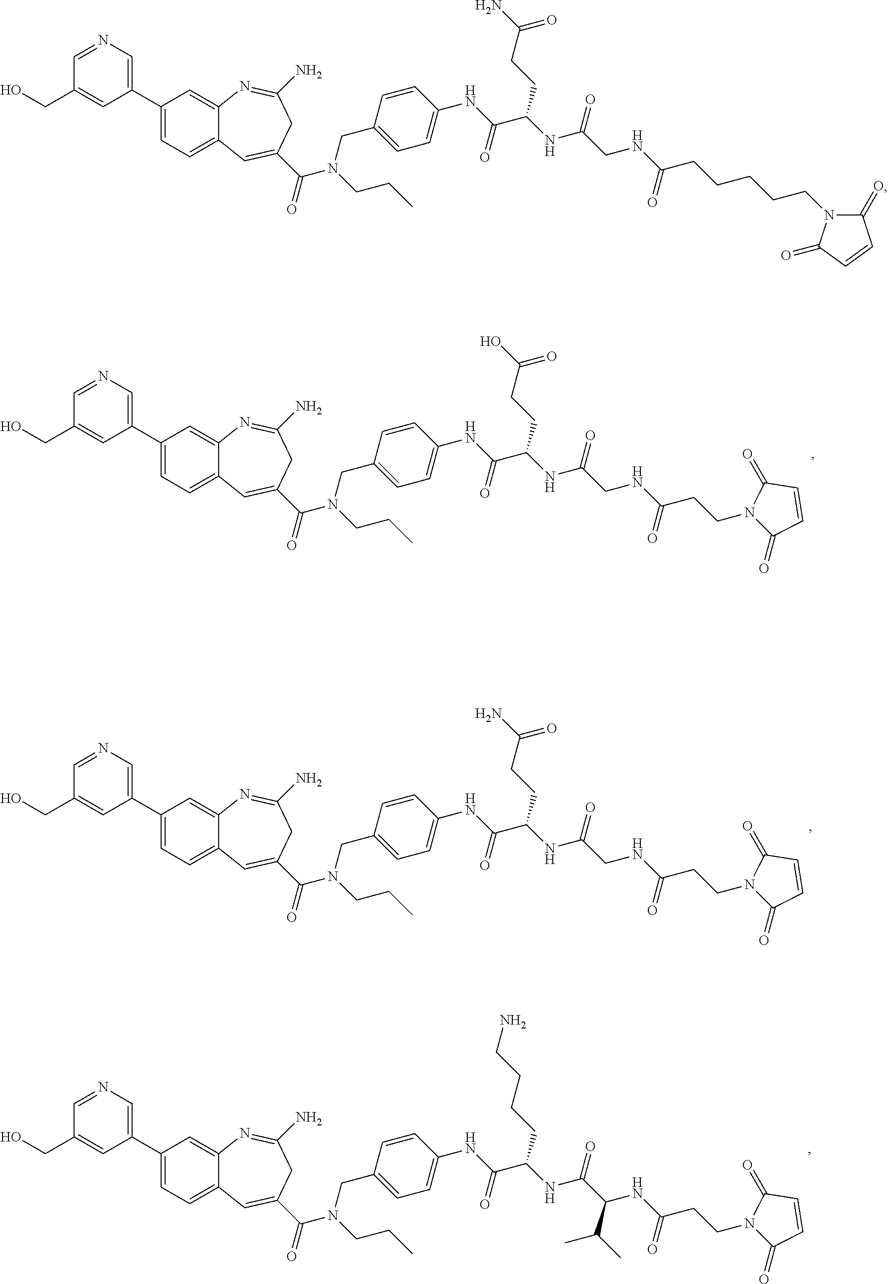 [SMILES: CCCN(Cc1ccc(NC(=O)[C@H](CCC(N)=O)NC(=O)CNC(=O)CCCCCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(N)=O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CCCCN)NC(=O)[C@@H](NC(=O)CCN2C(=O)C=CC2=O)C(C)C)cc1)C(=O)C1=Cc2ccc(-c3cncc(CO)c3)cc2N=C(N)C1]

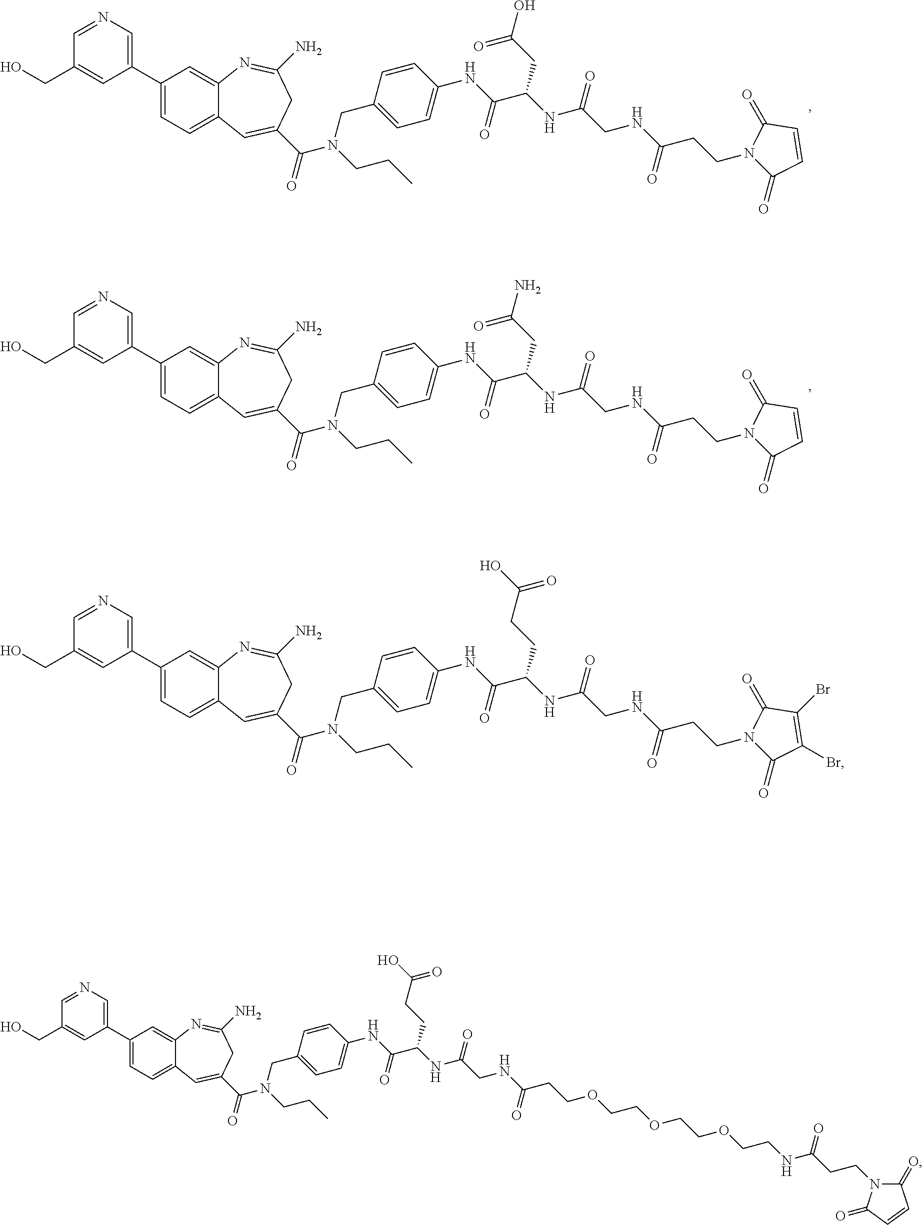 [SMILES: CCCN(Cc1ccc(NC(=O)[C@H](CC(=O)O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CC(N)=O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCN2C(=O)C(Br)=C(Br)C2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCOCCOCCOCCNC(=O)CCN2C(=O)C=CC2=O)cc1)C(=O)C1=Cc2ccc(-c3cncc(CO)c3)cc2N=C(N)C1]

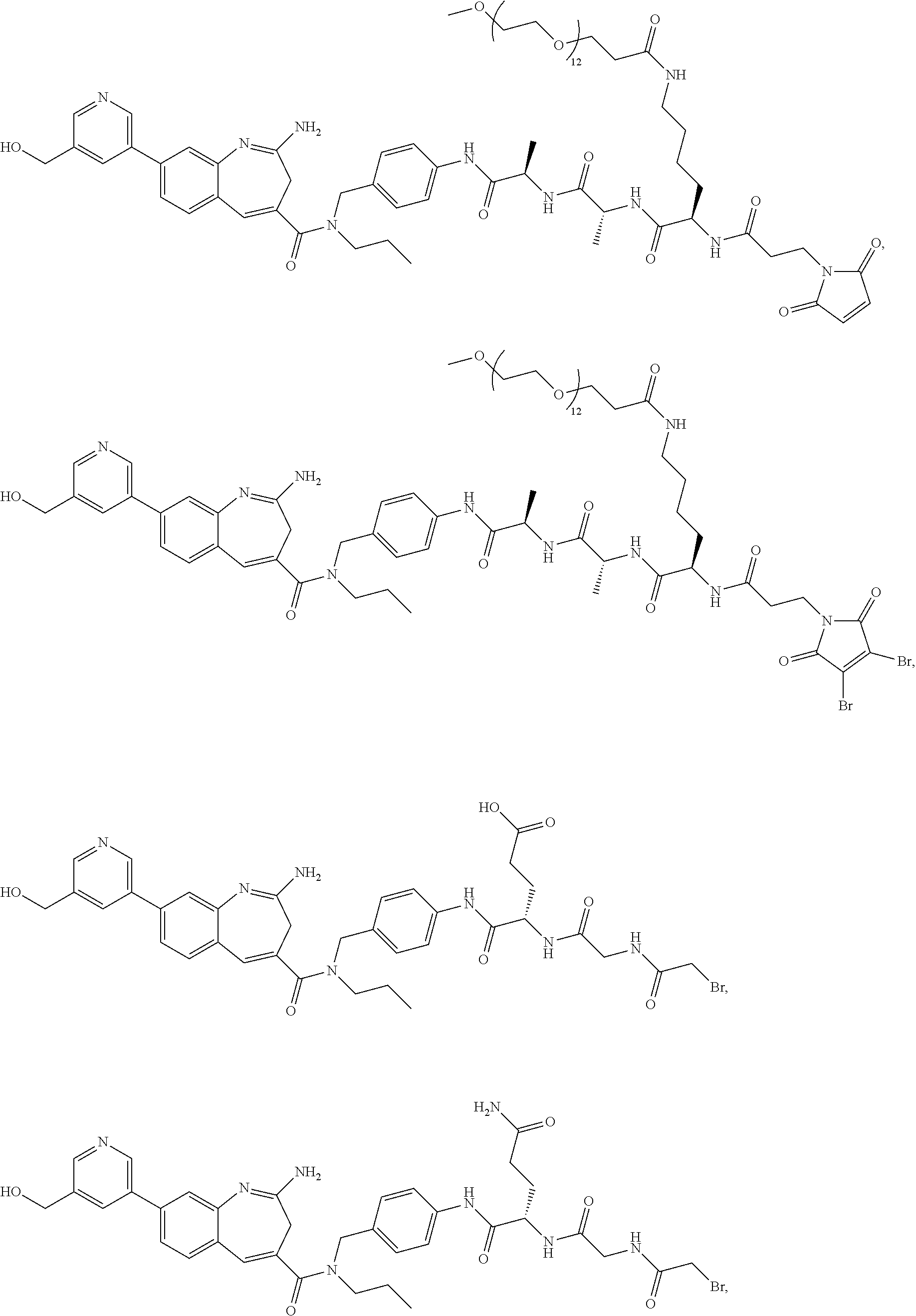

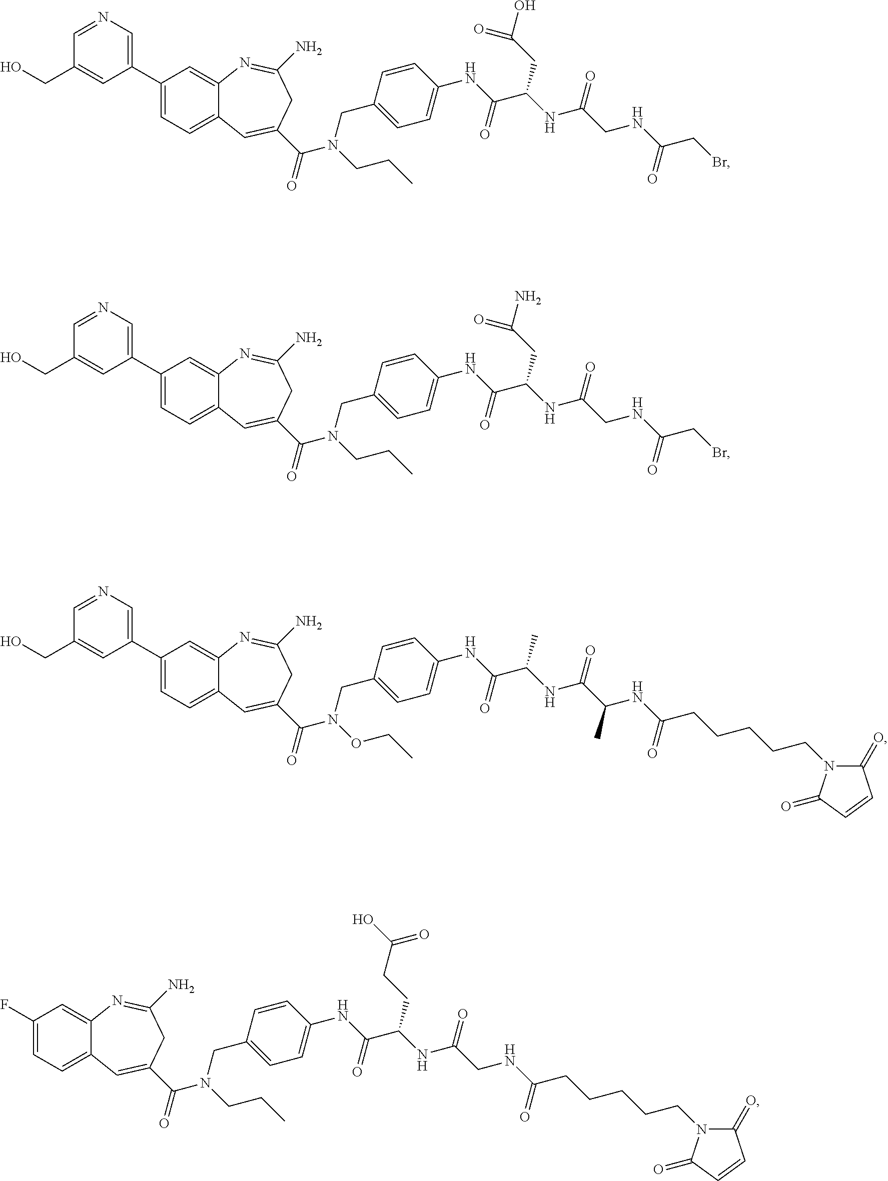 [SMILES: CCCN(Cc1ccc(NC(=O)[C@H](CC(=O)O)NC(=O)CNC(=O)CBr)cc1)C(=O)C1=Cc2ccc(-c3cncc(CO)c3)cc2N=C(N)C1 ; CCCN(Cc1ccc(NC(=O)[C@H](CC(N)=O)NC(=O)CNC(=O)CBr)cc1)C(=O)C1=Cc2ccc(-c3cncc(CO)c3)cc2N=C(N)C1 ; CCON(Cc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCCCCN2C(=O)C=CC2=O)cc1)C(=O)C1=Cc2ccc(F)cc2N=C(N)C1]

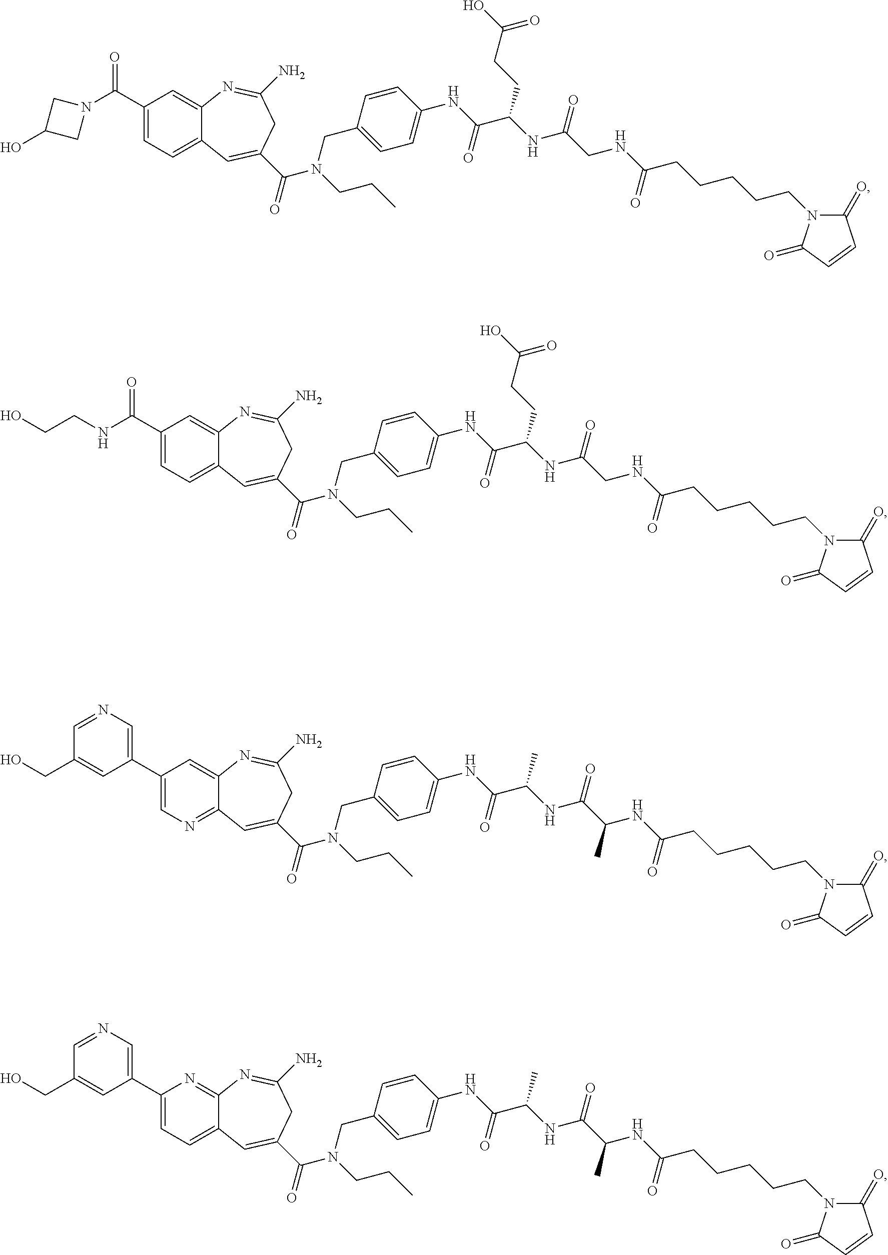 [SMILES: CCCN(Cc1ccc(NC(=O)[C@H](CCC(=O)O)NC(=O)CNC(=O)CCCCCN2C(=O)C=CC2=O)cc1)C(=O)C1=Cc2ccc(C(=O)N3CC(O)C3)cc2N=C(N)C1 ; CCCN(Cc1ccc(NC(=O)[C@H](CCC(=O)O)NC(=O)CNC(=O)CCCCCN2C(=O)C=CC2=O)cc1)C(=O)C1=Cc2ccc(C(=O)NCCO)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ncc(-c3cncc(CO)c3)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)nc2N=C(N)C1]

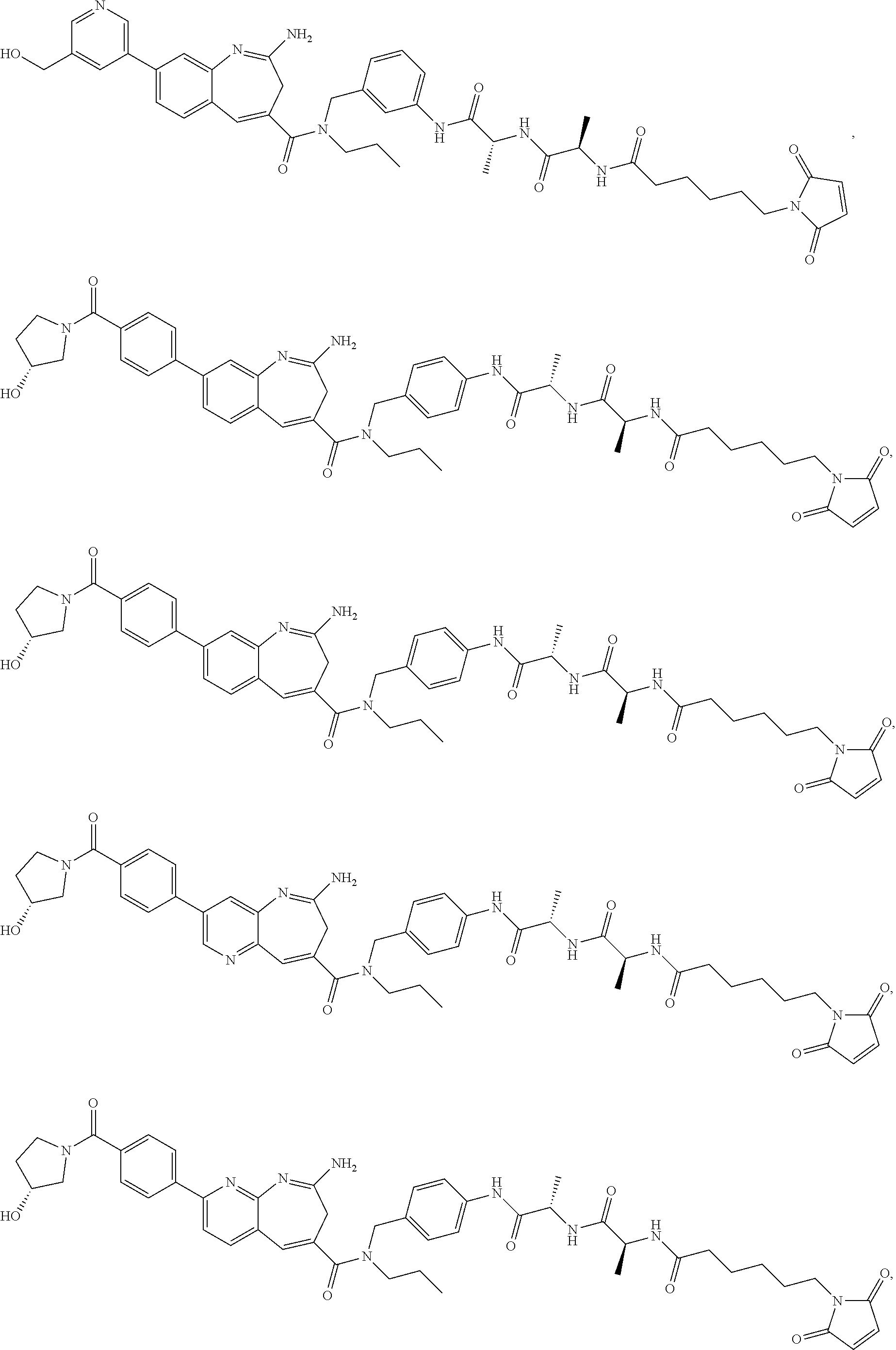

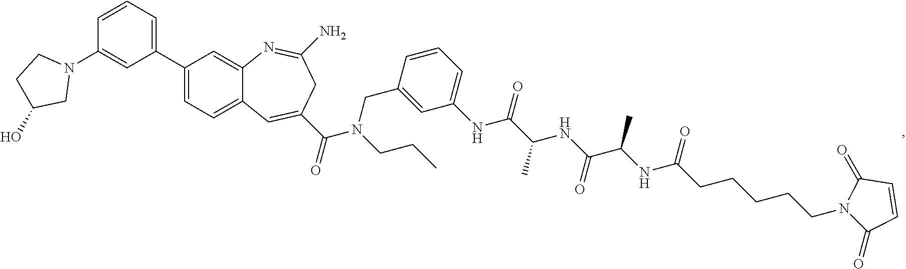 [SMILES: CCCN(Cc1cccc(NC(=O)[C@@H](C)NC(=O)[C@@H](C)NC(=O)CCCCCN2C(=O)C=CC2=O)c1)C(=O)C1=Cc2ccc(-c3cccc(N4CC[C@@H](O)C4)c3)cc2N=C(N)C1]

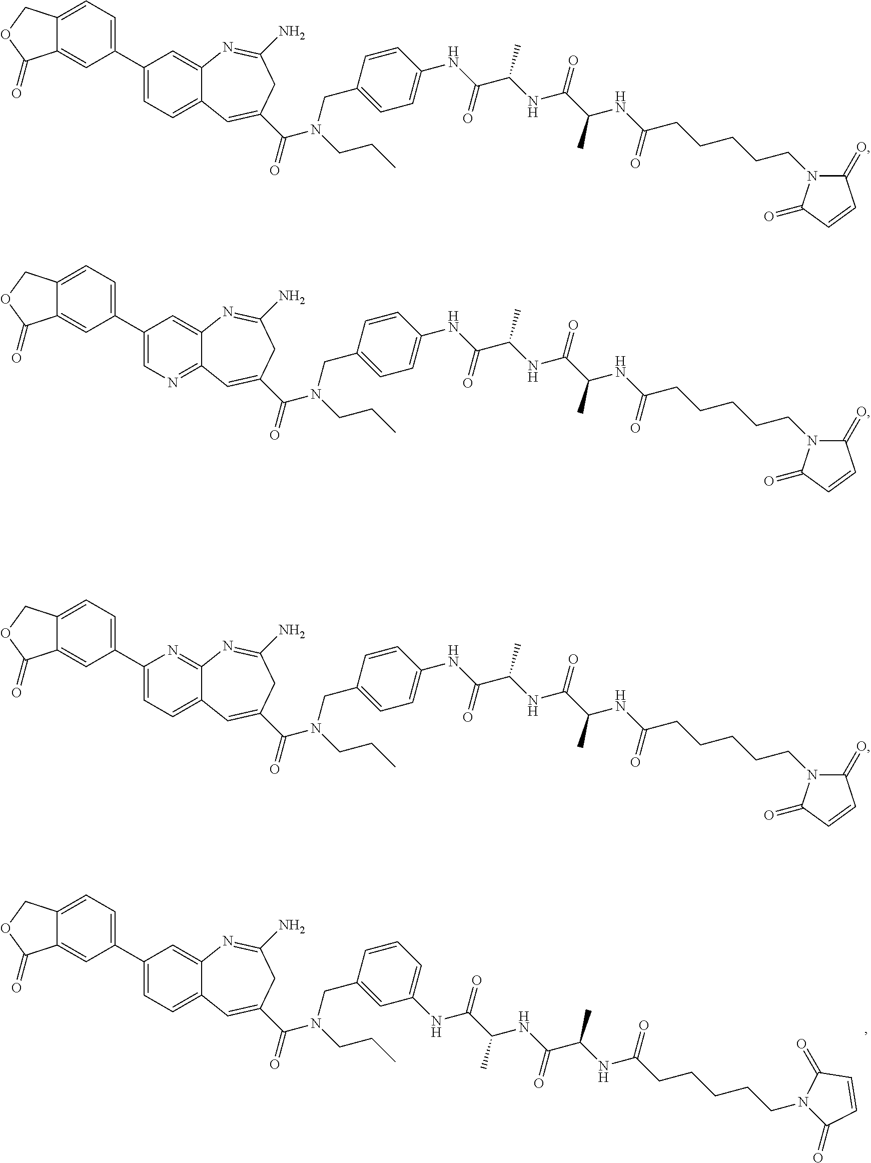 [SMILES: CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc4c(c3)C(=O)OC4)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ncc(-c3ccc4c(c3)C(=O)OC4)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc4c(c3)C(=O)OC4)nc2N=C(N)C1 ; CCCN(Cc1cccc(NC(=O)[C@@H](C)NC(=O)[C@@H](C)NC(=O)CCCCCN2C(=O)C=CC2=O)c1)C(=O)C1=Cc2ccc(-c3ccc4c(c3)C(=O)OC4)cc2N=C(N)C1]

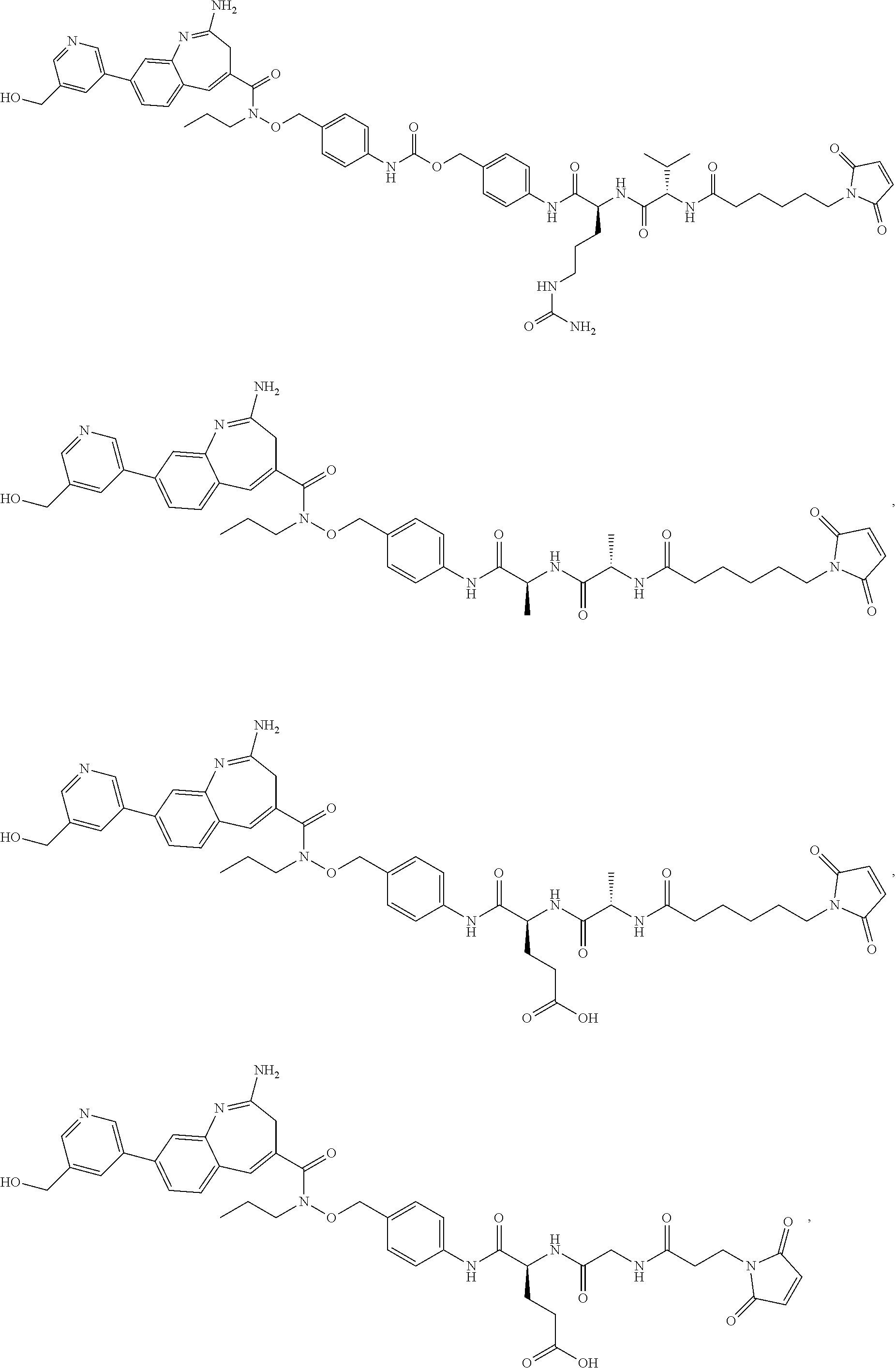 [SMILES: CCCN(OCc1ccc(NC(=O)OCc2ccc(NC(=O)[C@H](CCCNC(N)=O)NC(=O)[C@@H](NC(=O)CCCCCN3C(=O)C=CC3=O)C(C)C)cc2)cc1)C(=O)C1=Cc2ccc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(=O)O)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(=O)O)NC(=O)CNC(=O)CCN2C(=O)C=CC2=O)cc1)C(=O)C1=Cc2ccc(-c3cncc(CO)c3)cc2N=C(N)C1]

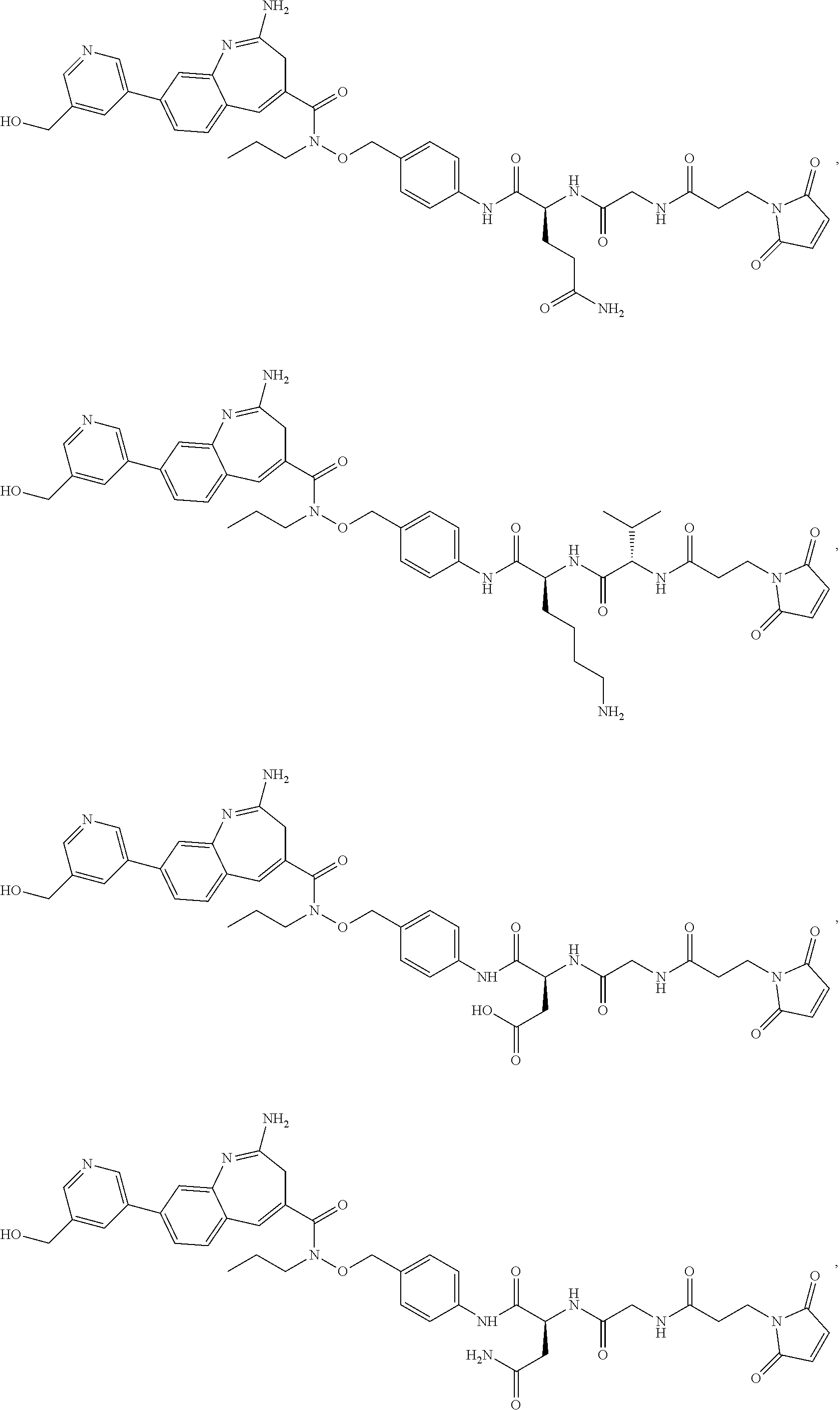 [SMILES: CCCN(OCc1ccc(NC(=O)[C@H](CCC(N)=O)NC(=O)CNC(=O)CCN2C(=O)C=CC2=O)cc1)C(=O)C1=Cc2ccc(-c3cncc(CO)c3)cc2N=C(N)C1 ; CCCN(OCc1ccc(NC(=O)[C@H](CCCCN)NC(=O)[C@@H](NC(=O)CCN2C(=O)C=CC2=O)C(C)C)cc1)C(=O)C1=Cc2ccc(-c3cncc(CO)c3)cc2N=C(N)C1 ; CCCN(OCc1ccc(NC(=O)[C@H](CC(=O)O)NC(=O)CNC(=O)CCN2C(=O)C=CC2=O)cc1)C(=O)C1=Cc2ccc(-c3cncc(CO)c3)cc2N=C(N)C1 ; CCCN(OCc1ccc(NC(=O)[C@H](CC(N)=O)NC(=O)CNC(=O)CCN2C(=O)C=CC2=O)cc1)C(=O)C1=Cc2ccc(-c3cncc(CO)c3)cc2N=C(N)C1]

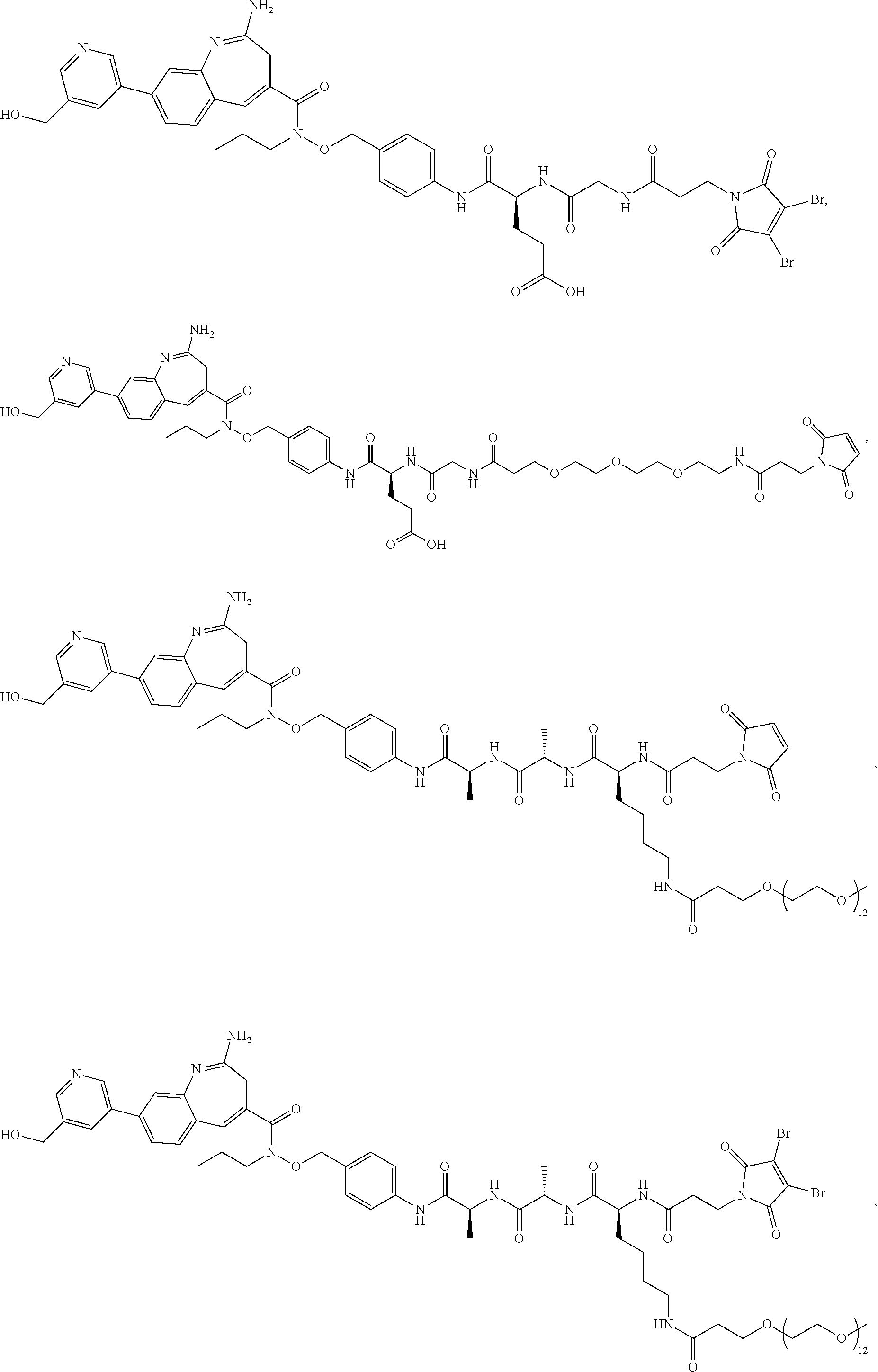

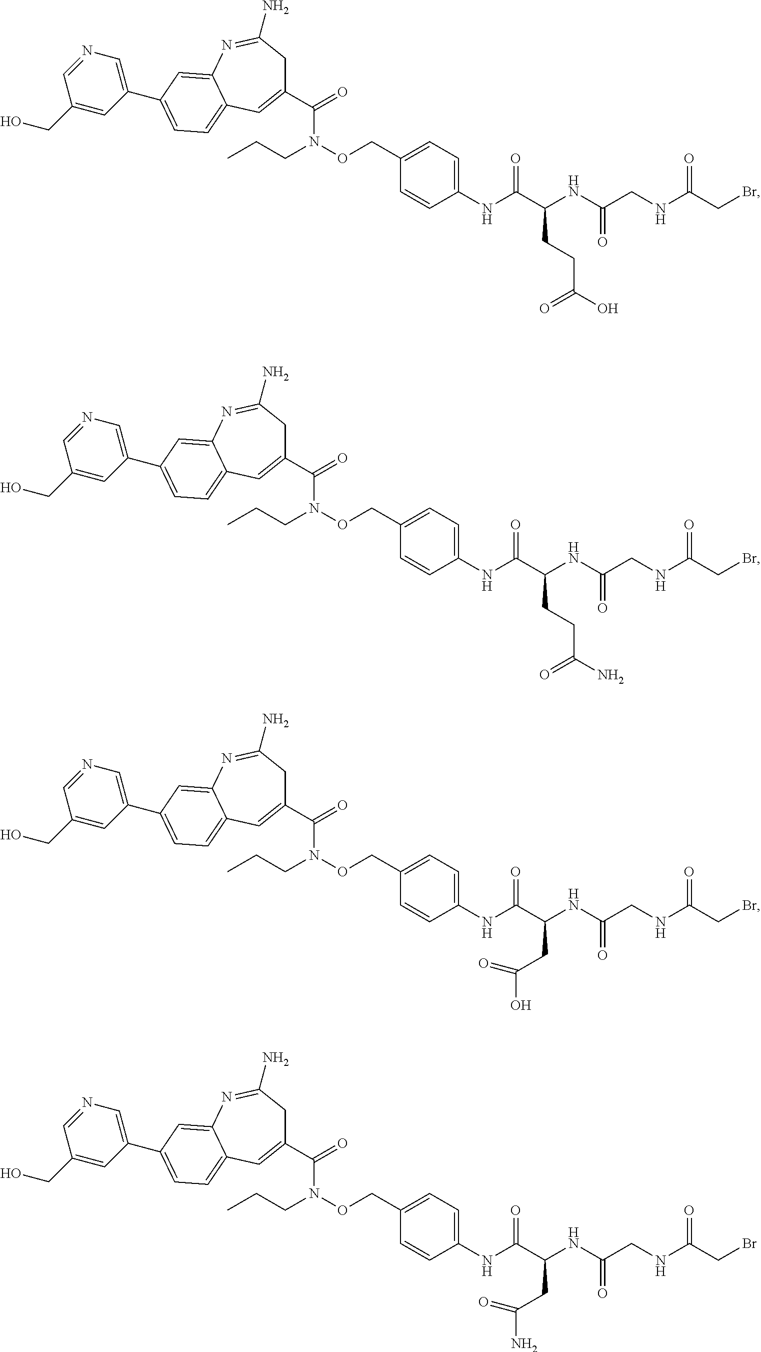 [SMILES: CCCN(OCc1ccc(NC(=O)[C@H](CCC(=O)O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CCC(N)=O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CC(=O)O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CC(N)=O)NC(=O)CNC(=O)CBr)cc1)C(=O)C1=Cc2ccc(-c3cncc(CO)c3)cc2N=C(N)C1]

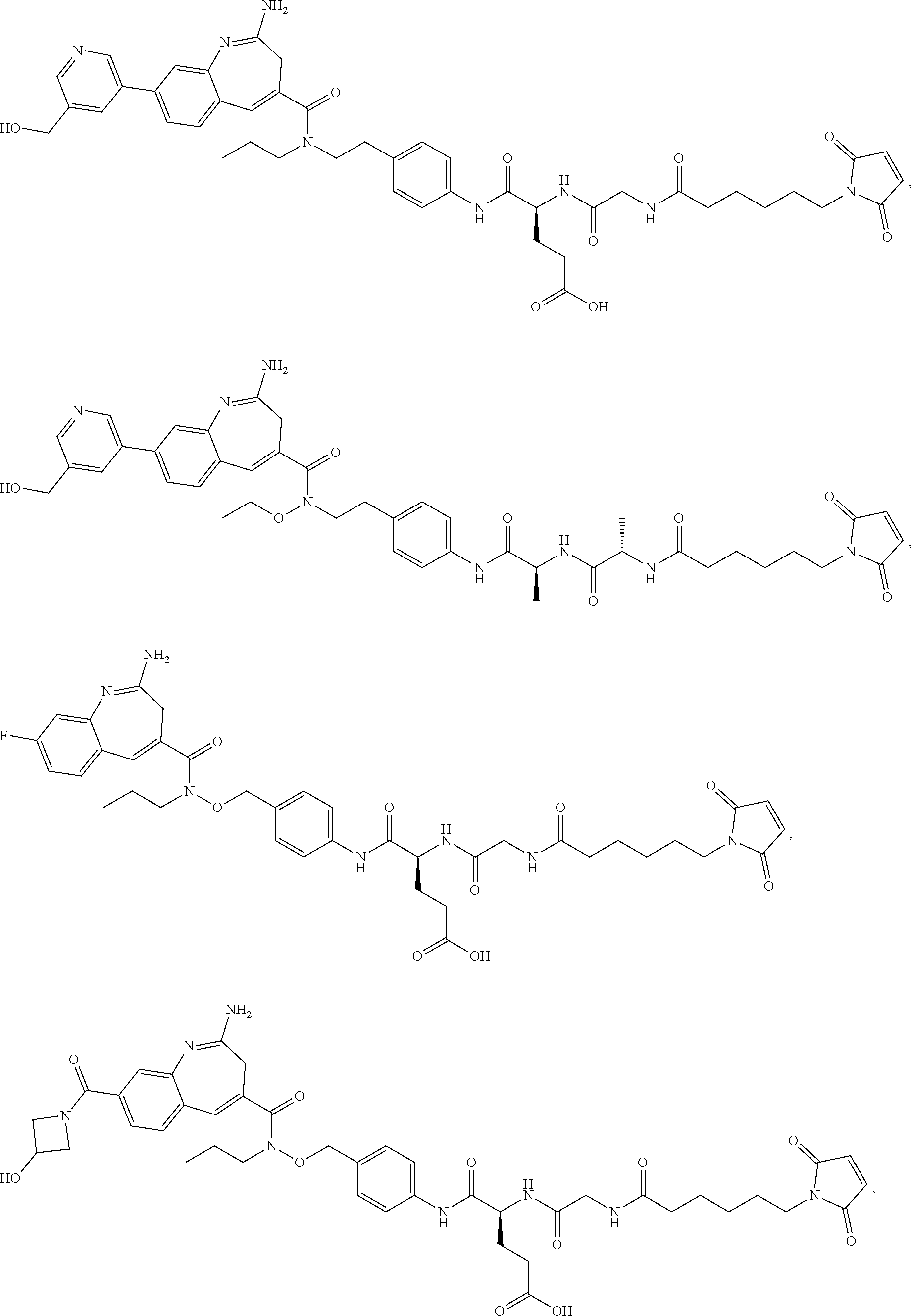 [SMILES: CCCN(CCc1ccc(NC(=O)[C@H](CCC(=O)O)NC(=O)CNC(=O)CCCCCN2C(=O)C=CC2=O)cc1)C(=O)C1=Cc2ccc(-c3cncc(CO)c3)cc2N=C(N)C1 ; CCON(CCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(=O)O)NC(=O)CNC(=O)CCCCCN2C(=O)C=CC2=O)cc1)C(=O)C1=Cc2ccc(F)cc2N=C(N)C1 ; CCCN(OCc1ccc(NC(=O)[C@H](CCC(=O)O)NC(=O)CNC(=O)CCCCCN2C(=O)C=CC2=O)cc1)C(=O)C1=Cc2ccc(C(=O)N3CC(O)C3)cc2N=C(N)C1]

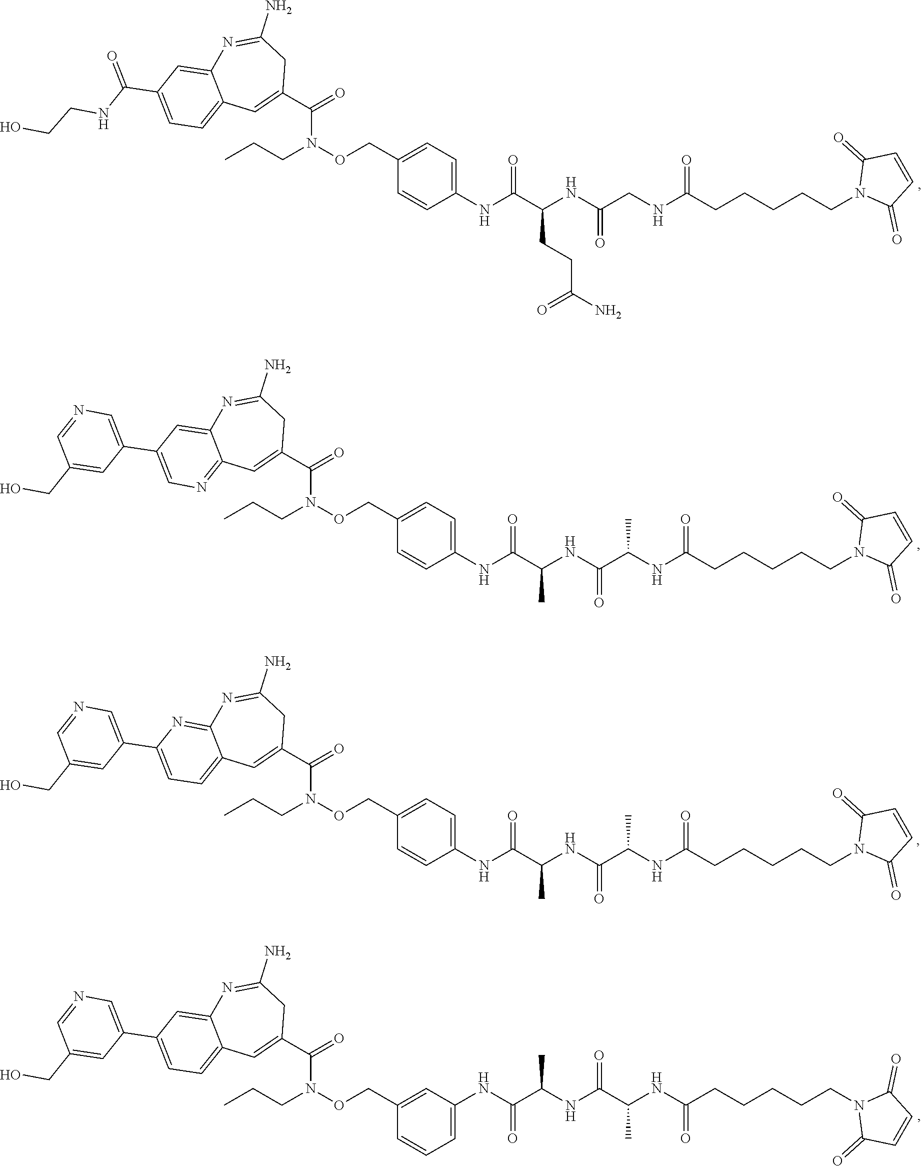 [SMILES: CCCN(OCc1ccc(NC(=O)[C@H](CCC(N)=O)NC(=O)CNC(=O)CCCCCN2C(=O)C=CC2=O)cc1)C(=O)C1=Cc2ccc(C(=O)NCCO)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)nc2N=C(N)C1 ; CCCN(OCc1cccc(NC(=O)[C@@H](C)NC(=O)[C@@H](C)NC(=O)CCCCCN2C(=O)C=CC2=O)c1)C(=O)C1=Cc2ccc(-c3cncc(CO)c3)cc2N=C(N)C1]

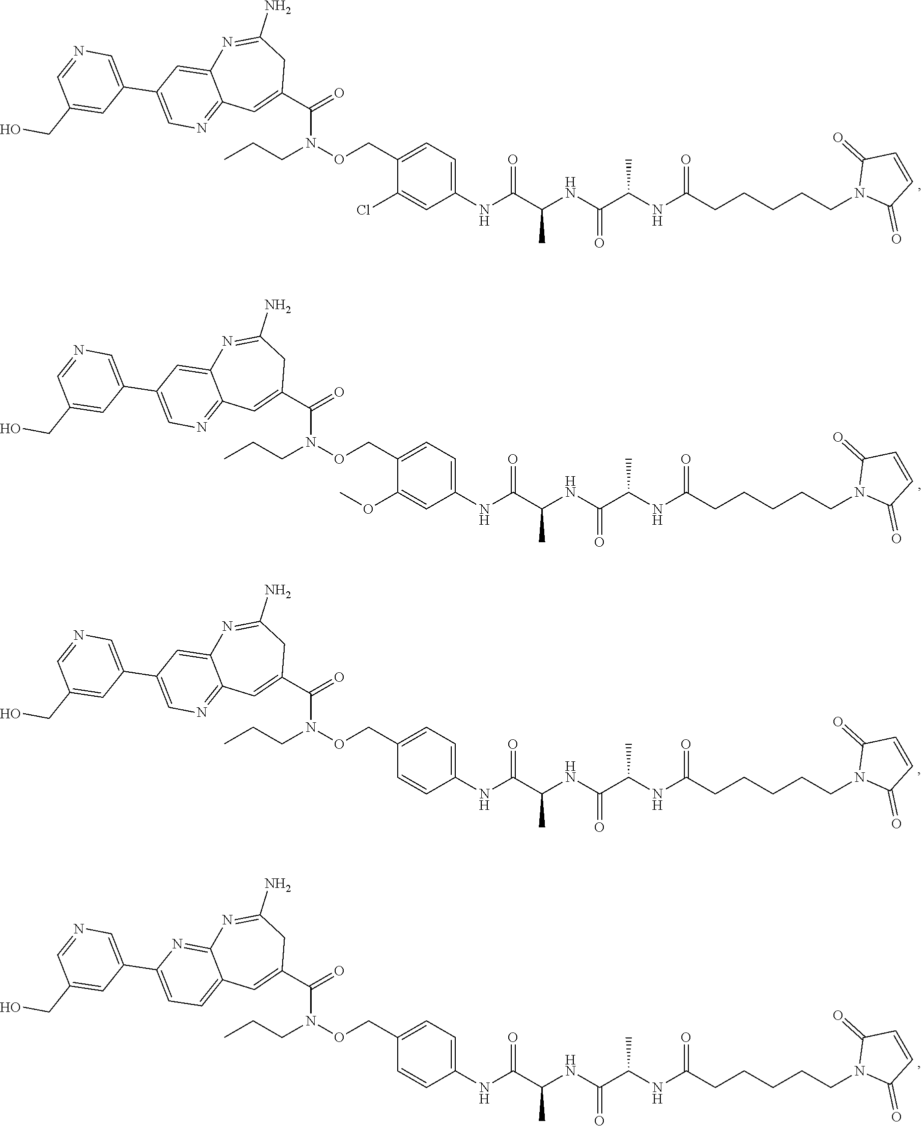 [SMILES: CCCN(OCc1ccc(NC(=O)[C@H](C)NC(=O)[C@H](C)NC(=O)CCCCCN2C(=O)C=CC2=O)cc1Cl)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1OC)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)nc2N=C(N)C1]

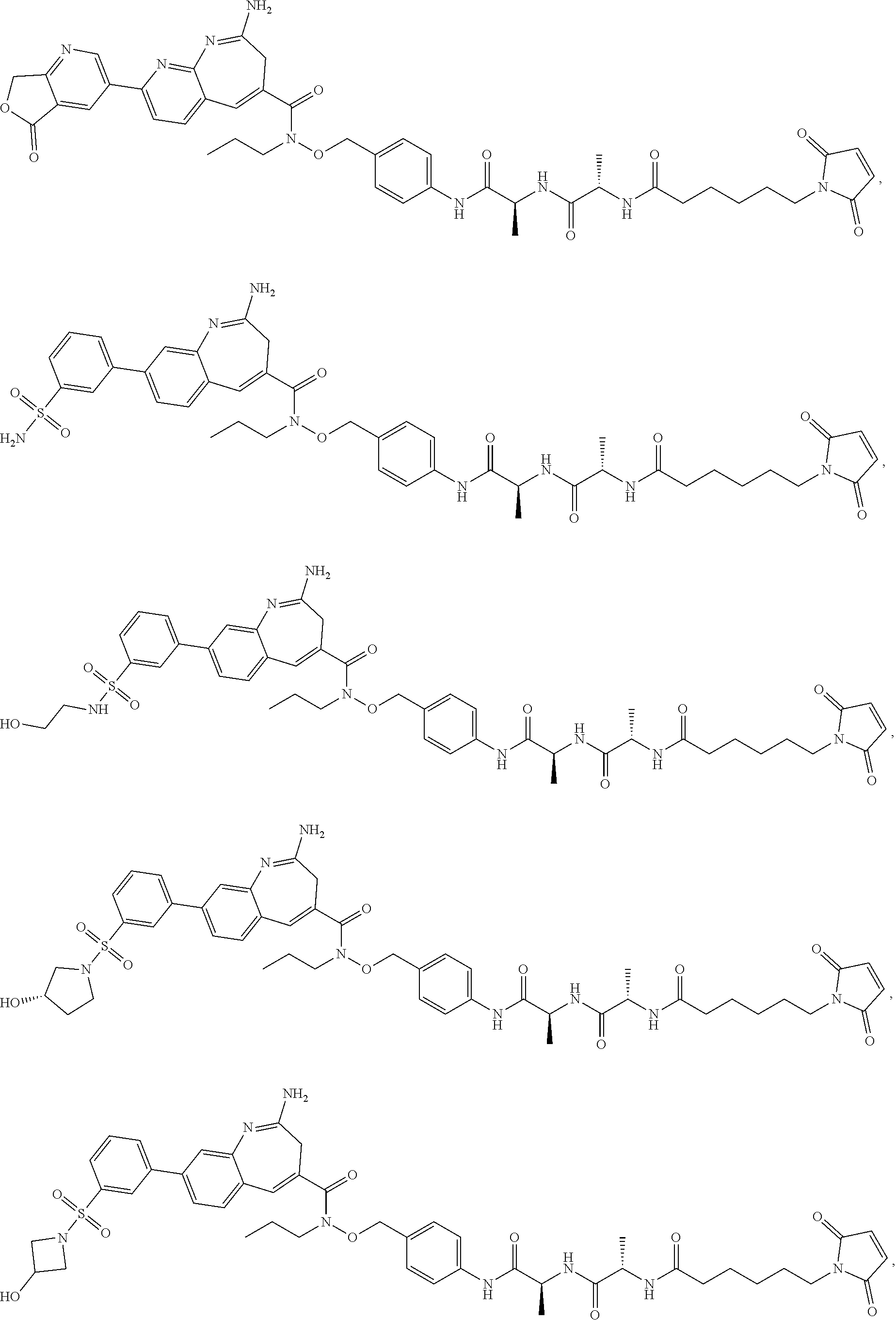

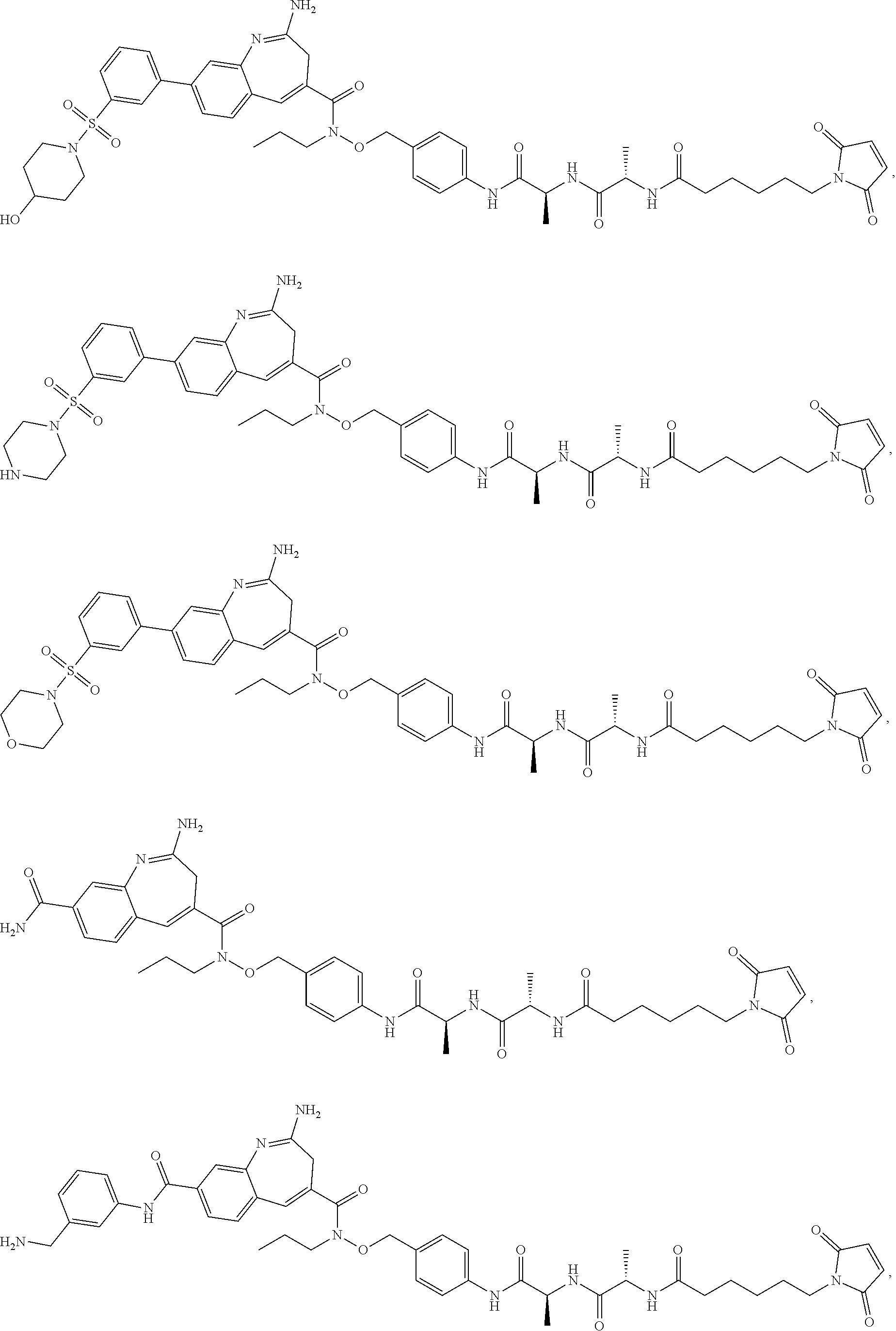

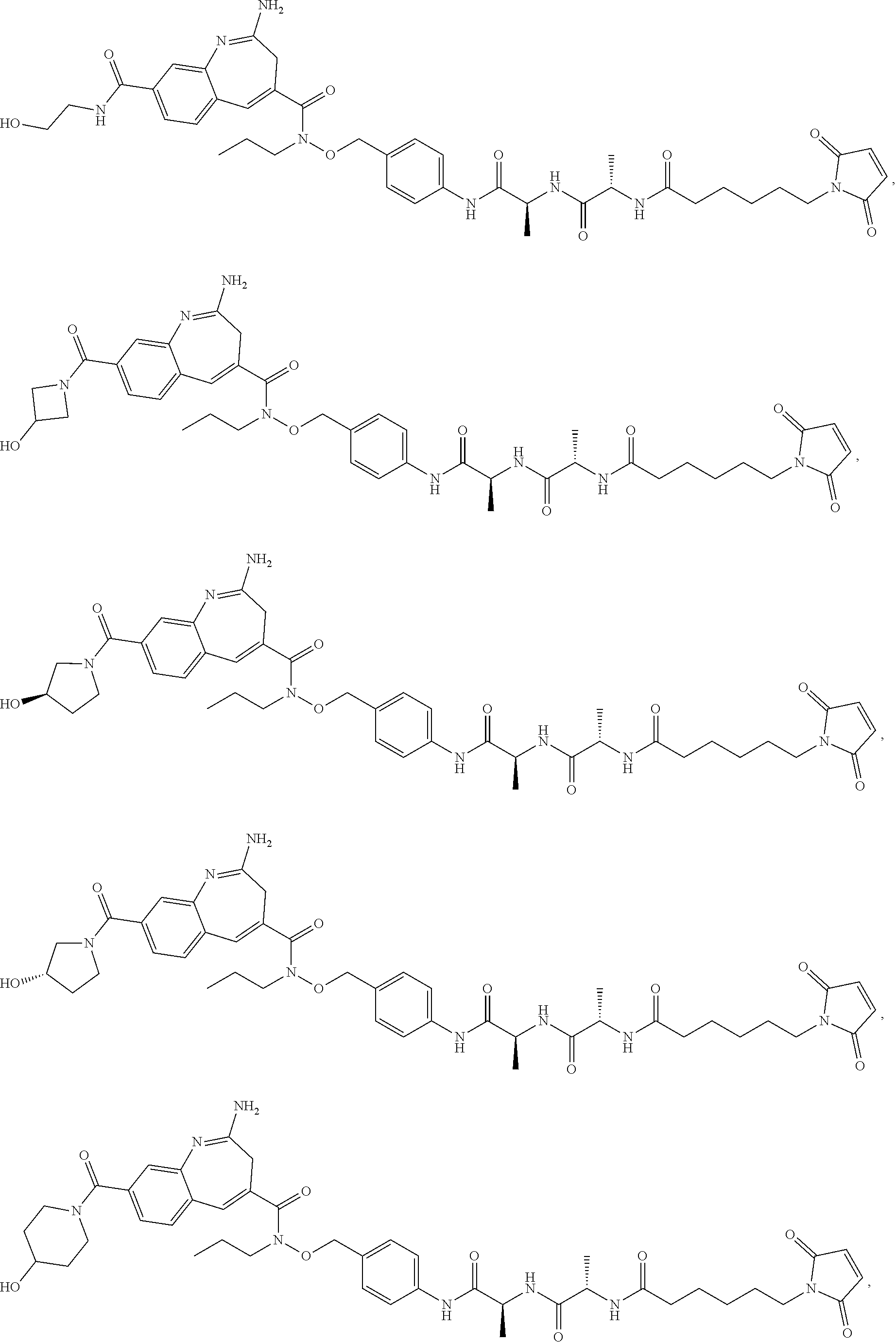

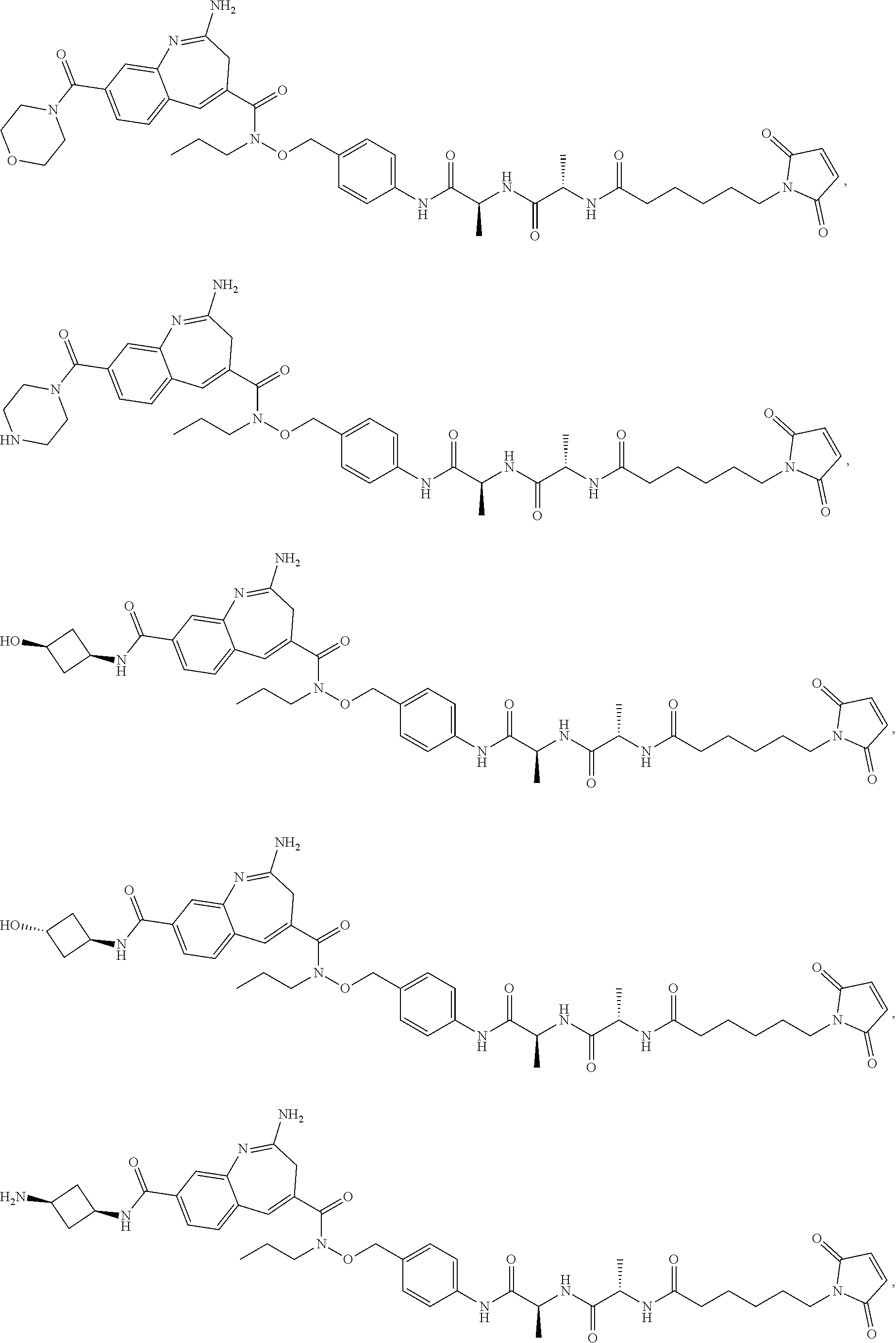

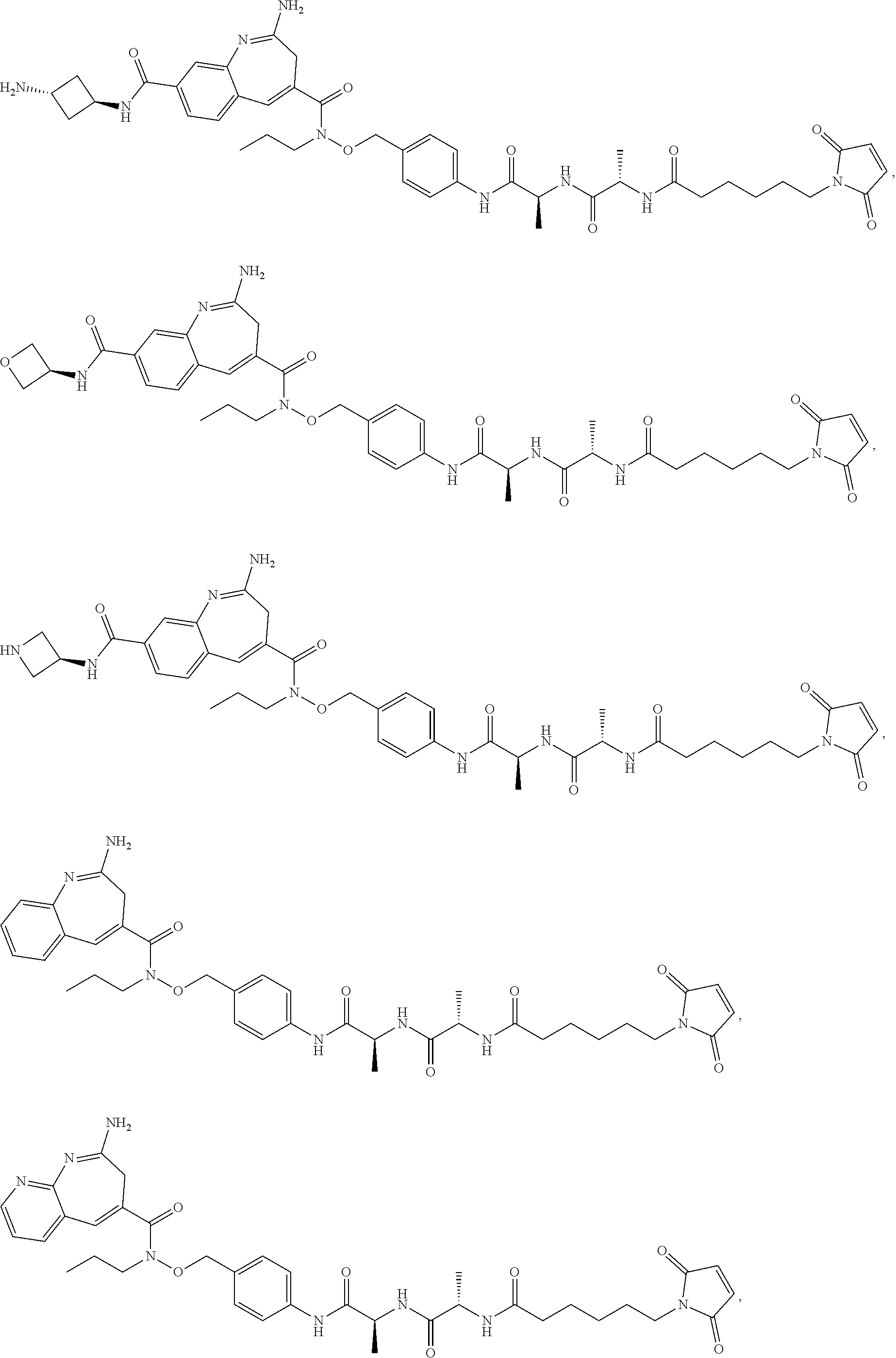

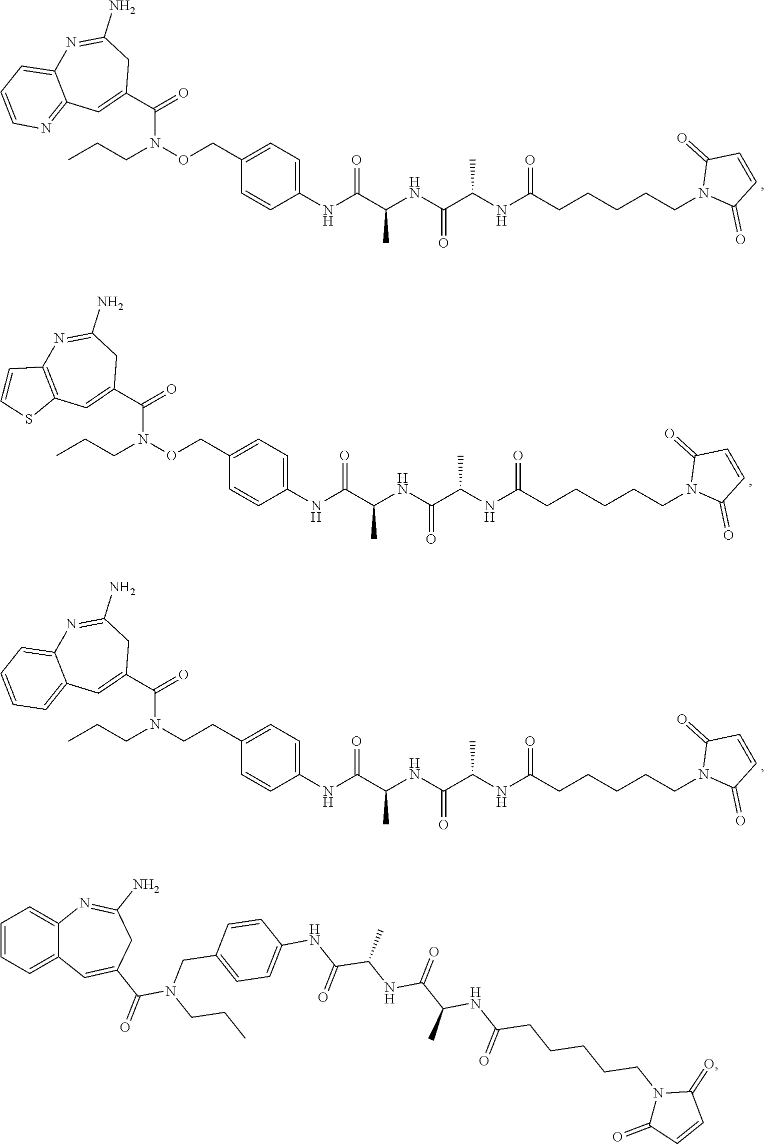 [SMILES: CCCN(OCc1ccc(NC(=O)[C@H](C)NC(=O)[C@H](C)NC(=O)CCCCCN2C(=O)C=CC2=O)cc1)C(=O)C1=Cc2ncccc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2sccc2N=C(N)C1 ; CCCN(CCc1ccc(NC(=O)[C@H](C)NC(=O)[C@H](C)NC(=O)CCCCCN2C(=O)C=CC2=O)cc1)C(=O)C1=Cc2ccccc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccccc2N=C(N)C1]

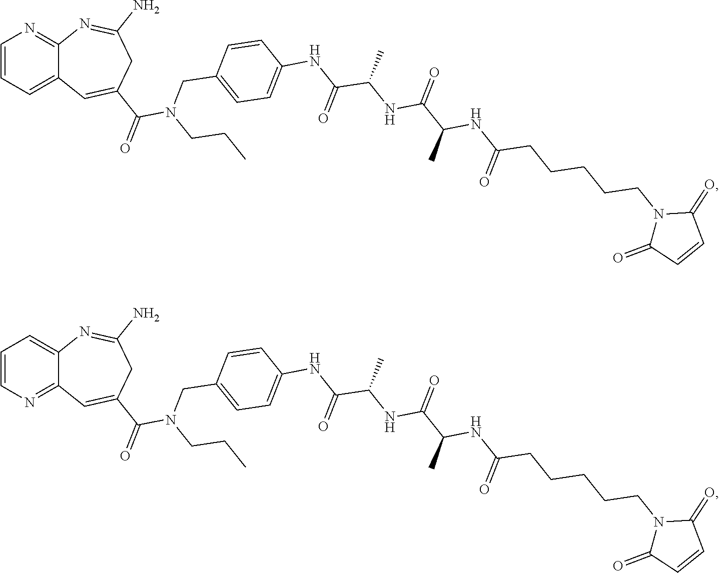 [SMILES: CCCN(Cc1ccc(NC(=O)[C@H](C)NC(=O)[C@H](C)NC(=O)CCCCCN2C(=O)C=CC2=O)cc1)C(=O)C1=Cc2cccnc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ncccc2N=C(N)C1]

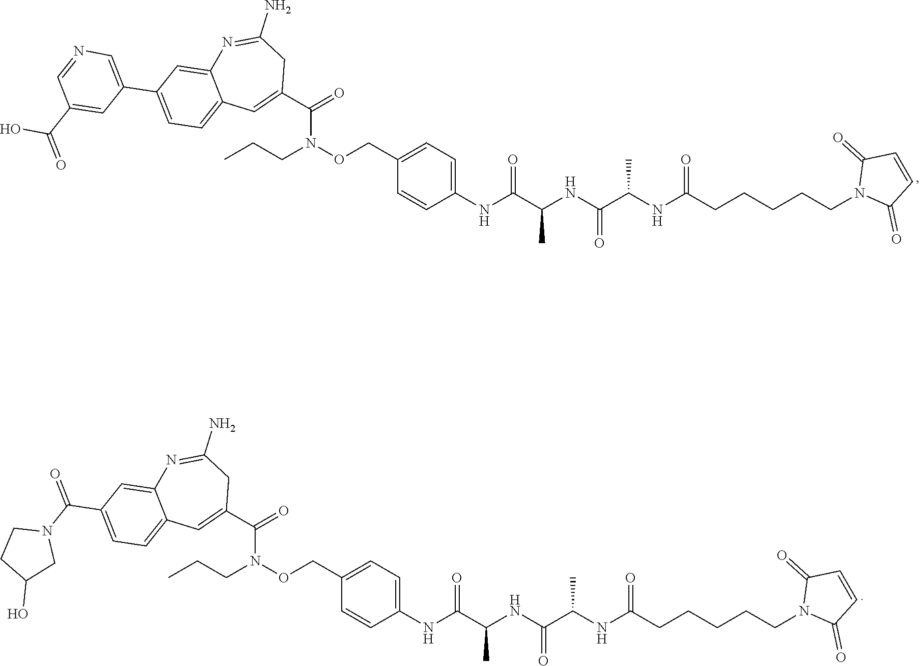 [SMILES: CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(C(=O)O)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N3CCC(O)C3)cc2N=C(N)C1]

In yet another aspect, the present disclosure provides a compound or a conjugate thereof, or an isomer thereof, or a mixture thereof, or a pharmaceutically acceptable salt thereof, wherein the compound comprises a structure represented by formula (III-1A):

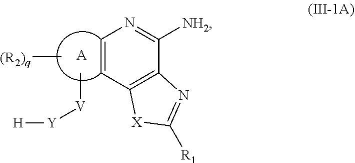

wherein
X is selected from —S— and —O—;
R₁ is selected from C₁-C₆ alkyl optionally substituted with one or more R₁₋₁, each R₁₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—,

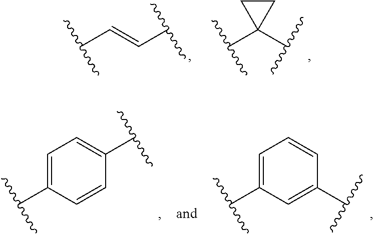

R_{V4} being selected from H, C₁-C₆ alkyl, and

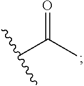 [SMILES: *C(C)=O]

Y is selected from —N(R_{Y1})—, —O—, —S—,

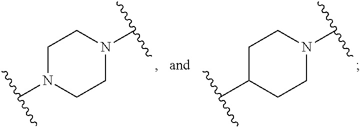 [SMILES: *N1CCN(*)CC1 ; *C1CCN(*)CC1]

R_{Y1} is selected from hydrogen and C₁-C₆ alkyl;
q is selected from 0, 1, 2, or 3;
p is selected from 1, 2, 3, 4, 5, 7, and 8.

In yet another aspect, the present disclosure provides a ligand-drug conjugate, or an isomer thereof, or a mixture thereof, or a pharmaceutically acceptable salt thereof, wherein the ligand-drug conjugate comprises a structure represented by formula (III-2A):

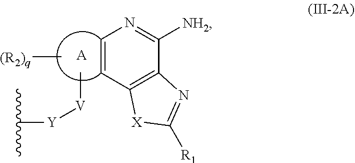

wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl; ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—,

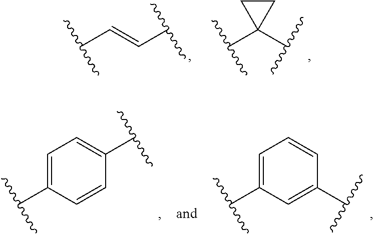

R_{V4} being selected from H, C₁-C₆ alkyl, and

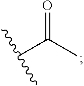 [SMILES: *C(C)=O]

Y is selected from —N(R_{Y1})—, —O—, —S—,

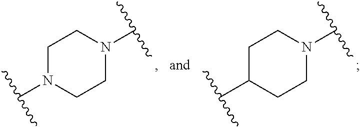 [SMILES: *N1CCN(*)CC1 ; *C1CCN(*)CC1]

R_{Y1} is selected from hydrogen and C₁-C₆ alkyl;
q is selected from 0, 1, 2, or 3;
p is selected from 1, 2, 3, 4, 5, 7, and 8;
the wavy line [symbol] in the general formula represents being directly linked to a ligand via a nitrogen atom or an oxygen atom on the Y group, or being linked to the ligand via a linker unit.

In some embodiments, in the ligand-drug conjugate, or the isomer thereof, or the mixture thereof, or the pharmaceutically acceptable salt thereof described herein, the ligand-drug conjugate comprises a structure represented by formula (III-2B):

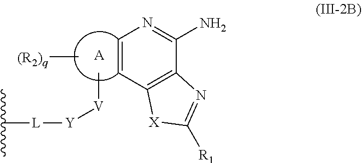

wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—,

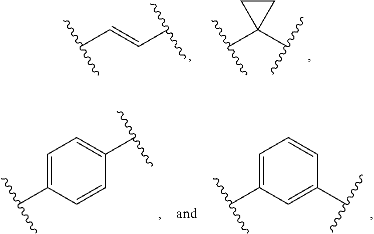

R_{V4} being selected from H, C₁-C₆ alkyl, and

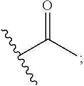 [SMILES: *C(C)=O]

Y is selected from —N(R_{Y1})—, —O—, —S—,

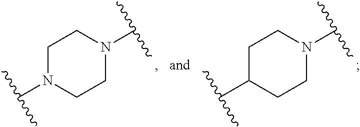 [SMILES: *N1CCN(*)CC1 ; *C1CCN(*)CC1]

R_{Y1} is selected from hydrogen and C₁-C₆ alkyl;
q is selected from 0, 1, 2, or 3;
p is selected from 1, 2, 3, 4, 5, 7, and 8;
L is a linker unit;
the wavy line [symbol] in the general formula represents being linked to the ligand via the nitrogen atom or the carbon atom on the L group.

In some embodiments, in the ligand-drug conjugate, or the isomer thereof, or the mixture thereof, or the pharmaceutically acceptable salt thereof described herein, the ligand-drug conjugate comprises a structure represented by formula (III-2C):

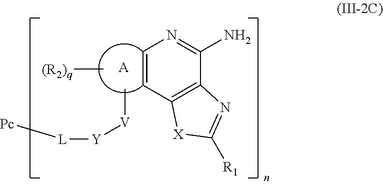

wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—,

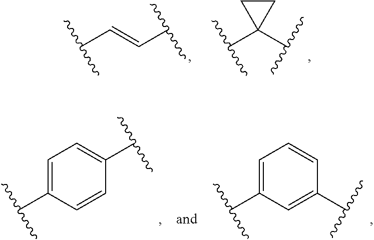

R_{V4} being selected from H, C₁-C₆ alkyl, and

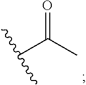 [SMILES: *C(C)=O]

Y is selected from —N(R_{Y1})—, —O—, —S—,

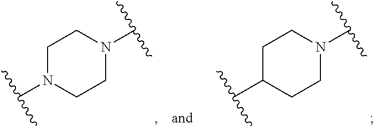 [SMILES: *N1CCN(*)CC1 ; *C1CCN(*)CC1]

R_{Y1} is selected from hydrogen and C₁-C₆ alkyl;
q is selected from 0, 1, 2, or 3;
p is selected from 1, 2, 3, 4, 5, 7, and 8;
L is a linker unit;
n is an integer or a decimal from 1 to 10;
Pc is a ligand.

In certain preferred embodiments of the present disclosure, certain groups in the structure represented by formula (III-1A), (III-2A), (III-2B) or (III-2C) are defined as follows, and groups not mentioned are as described in any one of the embodiments of the present application.

In some embodiments, in the structure represented by formula (III-1A), (III-2A), (III-2B) or (III-2C), R₁ is selected from C₁-C₆ alkyl optionally substituted with 1, 2, or 3 R₁₋₁, each R₁₋₁ being independently selected from: hydrogen, chlorine, bromine, iodine, hydroxy, amino, —CH₃, —CH₂CH₃, and

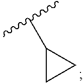 [SMILES: *C1CC1]

the methylene units of R₁ are each independently unreplaced or replaced by a group selected from: —O—, —N(CH₃)—, and —NH—.

In some embodiments, in the structure represented by formula (III-1A), (III-2A), (III-2B) or (III-2C), R₁ is selected from

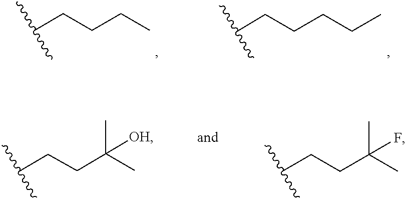

wherein the methylene units of R₁ are each independently unreplaced or replaced by a group selected from: —O—, —N(CH₃)—, and —NH—.

In some embodiments, in the structure represented by formula (III-1A), (III-2A), (III-2B) or (III-2C), R₁ is selected from

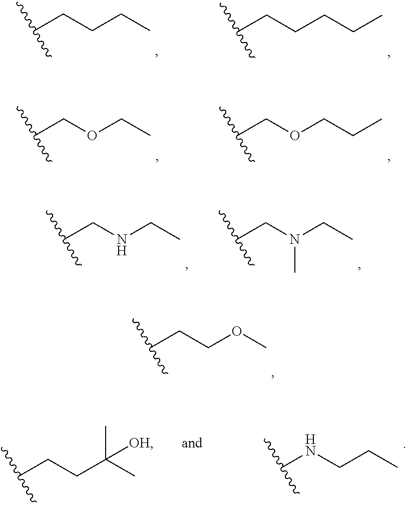

For example, R₁ is selected from

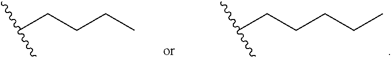

In some embodiments, in the structure represented by formula (III-1A), (III-2A), (III-2B) or (III-2C), R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂₋₁, each R₂₋₁ being independently selected from: hydrogen, halogen, hydroxy, and amino.

In some embodiments, in the structure represented by formula (III-1A), (III-2A), (III-2B) or (III-2C), R₂ is selected from hydrogen, chlorine, bromine, iodine, hydroxy, amino, sulfhydryl, —CH₃, —CH₂CH₃, and —OCH₃. For example, R₂ is selected from H.

In some embodiments, in the structure represented by formula (III-1A), (III-2A), (III-2B) or (III-2C), ring A is selected from phenyl, pyridinyl, pyrrolyl, thienyl, furanyl, pyridazinyl, pyrimidinyl, and pyrazinyl.

In some embodiments, in the structure represented by formula (III-1A), (III-2A), (III-2B) or (III-2C), ring A is selected from phenyl or pyridinyl. For example, ring A is selected from phenyl.

In some embodiments, in the structure represented by formula (III-1A), (III-2A), (III-2B) or (III-2C), V is selected from —(C(Rvi)(R_{V2}))ₚ—, wherein R_{A1} and R_{A2} are each independently selected from hydrogen, chlorine, bromine, iodine, hydroxy, amino, —CH₃, —CH₂CH₃, and

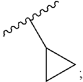 [SMILES: *C1CC1]

the methylene units of V are each independently unreplaced or replaced by a group selected from: —O—, —NH—, —CO—, —NHCO—,

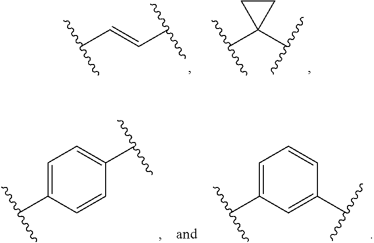

In some embodiments, in the structure represented by formula (III-1A), (III-2A), (III-2B) or (III-2C), V is selected from

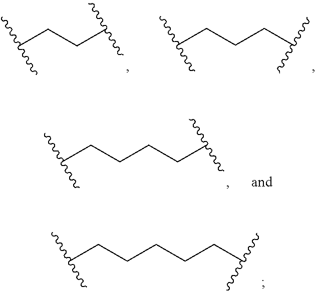

the methylene units of V are each independently unreplaced or replaced by a group selected from: —O—, —NH—, —CO—, —NHCO—,

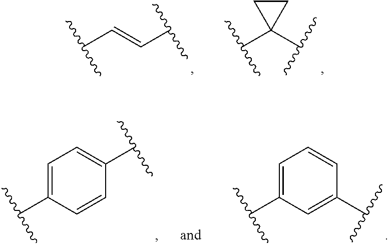

In some embodiments, in the structure represented by formula (III-1A), (III-2A), (III-2B) or (III-2C), V is selected from

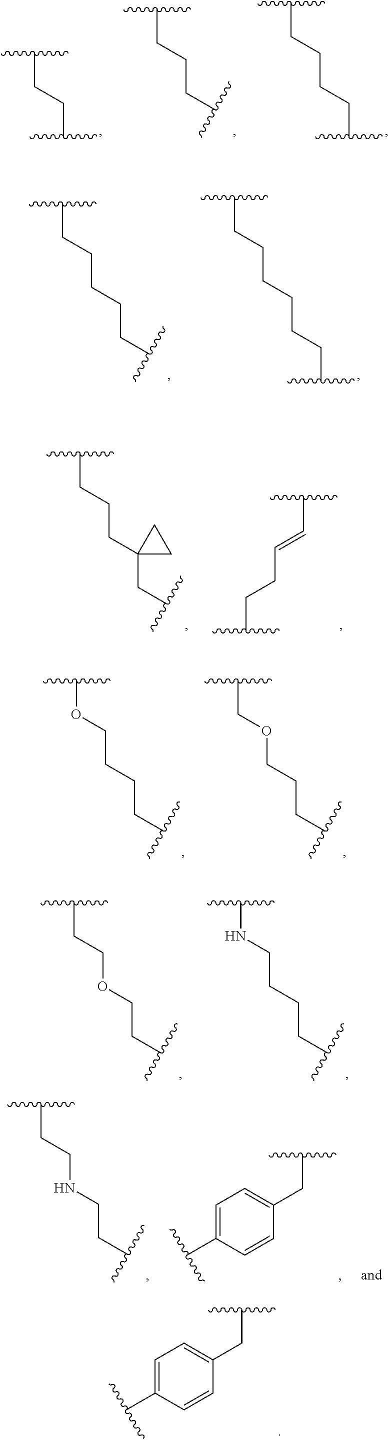

For example, V is selected from

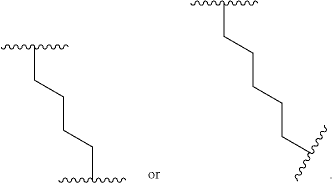 [SMILES: *CCCC* ; *CCCCC*]

In some embodiments, in the structure represented by formula (III-1A), (III-2A), (III-2B) or (III-2C), Y is selected from —NH—, —O—, —S—,

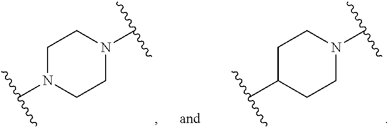 [SMILES: *N1CCN(*)CC1 ; *C1CCN(*)CC1]

In some embodiments, in the structure represented by formula (III-1A), (III-2A), (III-2B) or (III-2C) the structural unit

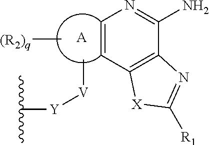

is selected from:

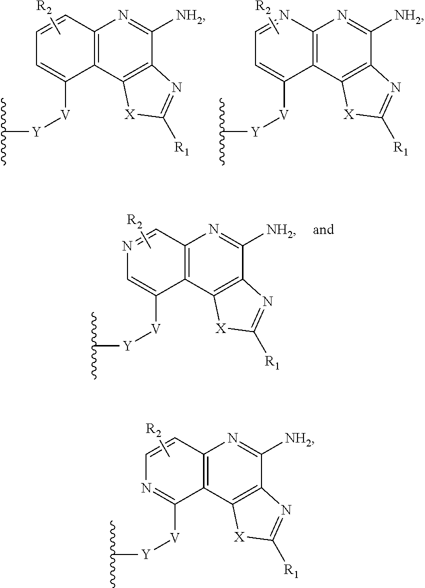

preferably selected from

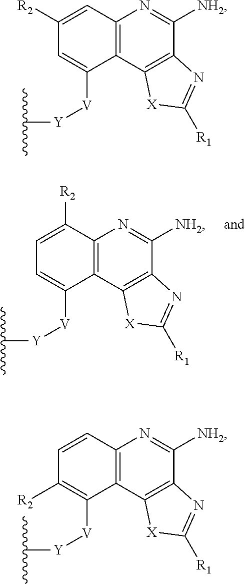

wherein
X is selected from —S— and —O—; R₁, R₂, V, and Y are as described in any one of the embodiments of (III-1A), (III-2A), (III-2B) and (III-2C) of the present application.

In some embodiments, in the structures represented by formulas (III-2B) and (III-2C), the linker unit L is -Tr-L₃-L₂-L_{1c}-, wherein
Tr is selected from

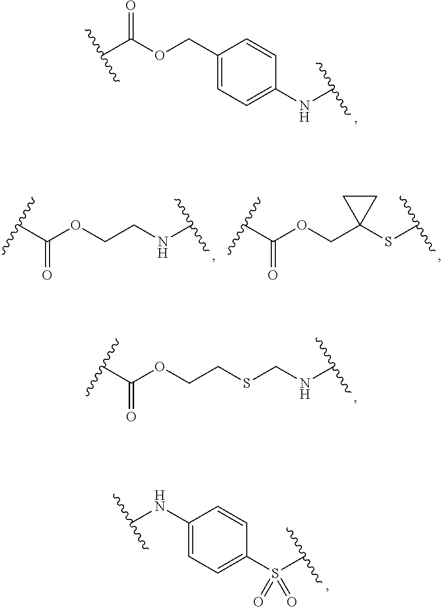 [SMILES: *Nc1ccc(COC(*)=O)cc1 ; *NCCOC(*)=O ; *SC1(COC(*)=O)CC1 ; *NCSCCOC(*)=O ; *Nc1ccc(S(*)(=O)=O)cc1]

and a single bond;
L_{1c} is selected from

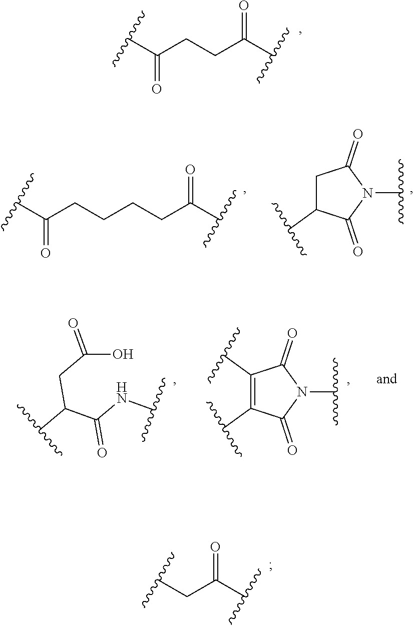 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]

L₂ is selected from

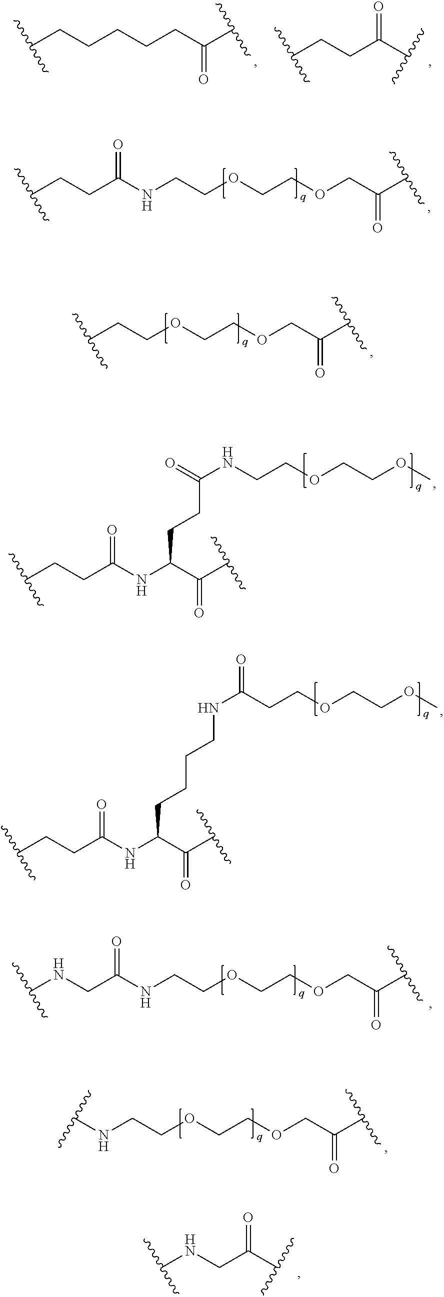

and a single bond, wherein q is an integer from 1 to 20;
L₃ is selected from a single bond or a peptide residue consisting of 1-4 amino acids, wherein the peptide residue is a peptide residue formed by an amino acid selected from the group consisting of: valine, lysine, cysteine, alanine, glycine, glutamic acid, phenylalanine, and serine.

In some embodiments, in the structures represented by formulas (III-2B) and (III-2C), L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In some embodiments, in the structures represented by formulas (III-2B) and (III-2C), the linker unit L is -L₃-L₂-L_{1c}-;
L_{1c} is selected from

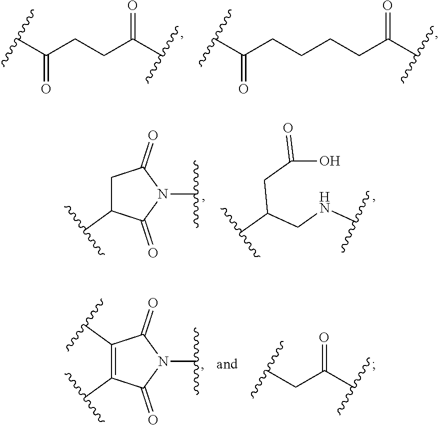 [SMILES: *C(=O)CCC(*)=O ; *C(=O)CCCCC(*)=O ; *C1CC(=O)N(*)C1=O ; *NCC(*)CC(=O)O ; *C1=C(*)C(=O)N(*)C1=O ; *CC(*)=O]

L₂ is selected from

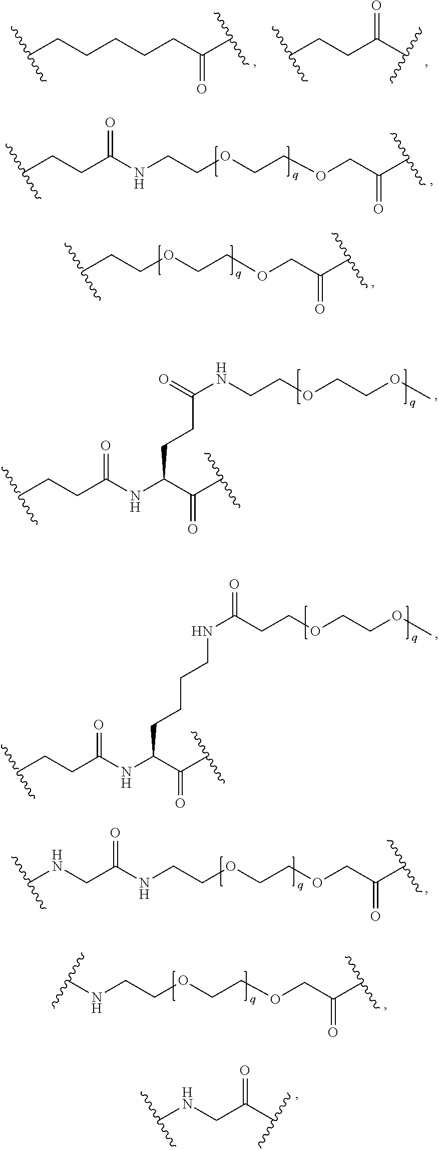

and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In some embodiments, in the structures represented by formulas (III-2B) and (III-2C), the linker unit L is -L₃-L₂-L_{1c}- or -Tr-L₃-L₂-L_{1c}-, wherein the L₃ end or the Tr end is connected to the nitrogen atom of the Z group, and the L_{1c} end is connected to the ligand.

In some embodiments, in the structures represented by formulas (III-2B) and (III-2C), the linker unit L is selected from:

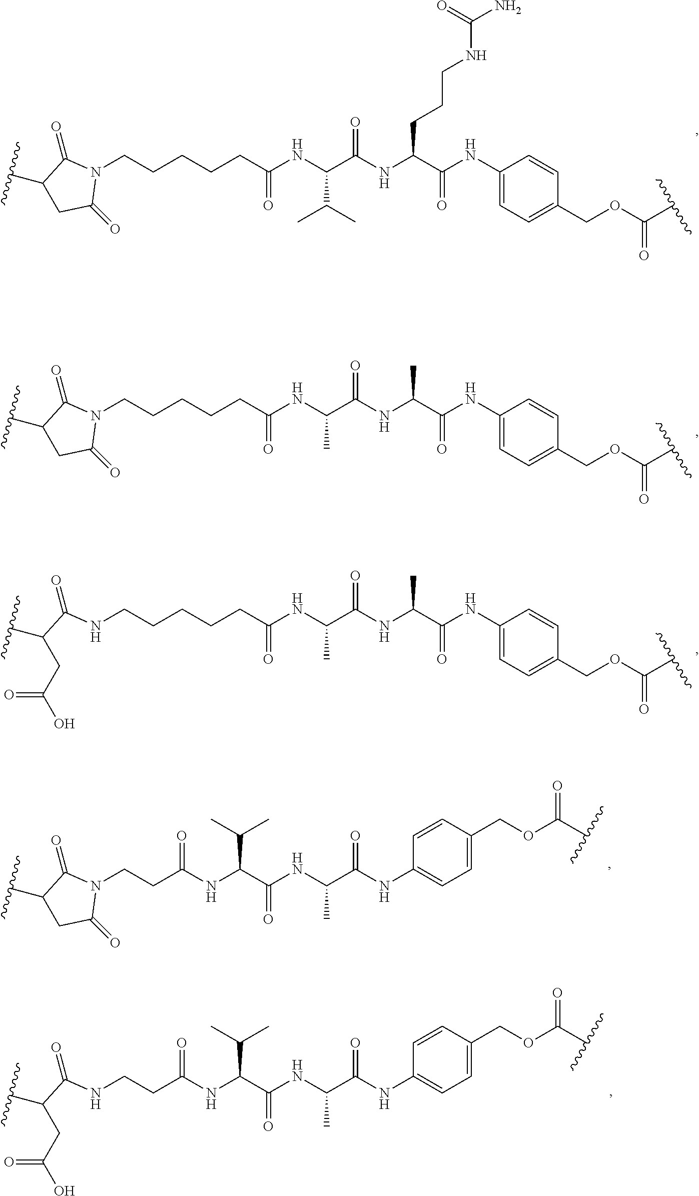 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)OCc1ccc(NC(=O)[C@H](C)NC(=O)[C@H](C)NC(=O)CCCCCN2C(=O)CC(*)C2=O)cc1 ; *C(=O)OCc1ccc(NC(=O)[C@H](C)NC(=O)[C@H](C)NC(=O)CCCCCNC(=O)C(*)CC(=O)O)cc1 ; *C(=O)OCc1ccc(NC(=O)[C@H](C)NC(=O)[C@@H](NC(=O)CCN2C(=O)CC(*)C2=O)C(C)C)cc1 ; *C(=O)OCc1ccc(NC(=O)[C@H](C)NC(=O)[C@@H](NC(=O)CCNC(=O)C(*)CC(=O)O)C(C)C)cc1]

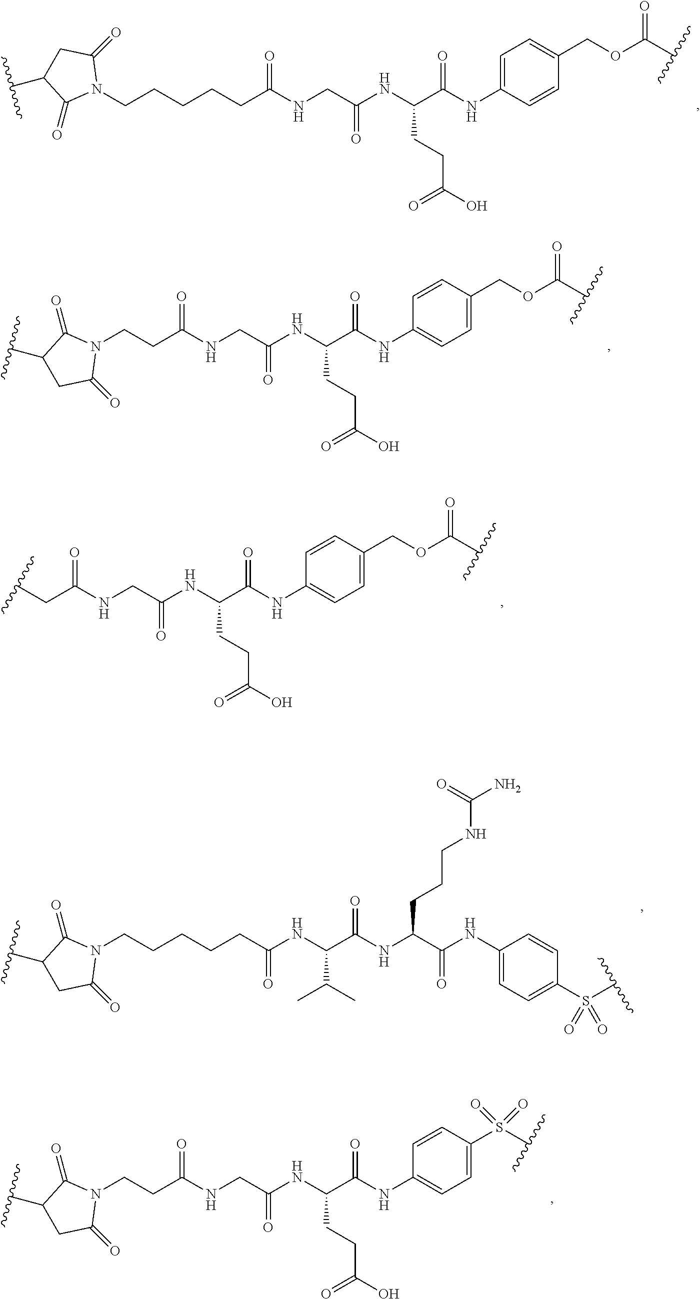 [SMILES: *C(=O)OCc1ccc(NC(=O)[C@H](CCC(=O)O)NC(=O)CNC(=O)CCCCCN2C(=O)CC(*)C2=O)cc1 ; *C(=O)OCc1ccc(NC(=O)[C@H](CCC(=O)O)NC(=O)CNC(=O)CCN2C(=O)CC(*)C2=O)cc1 ; *CC(=O)NCC(=O)N[C@@H](CCC(=O)O)C(=O)Nc1ccc(COC(*)=O)cc1 ; *C1CC(=O)N(CCCCCC(=O)N[C@H](C(=O)N[C@@H](CCCNC(N)=O)C(=O)Nc2ccc(S(*)(=O)=O)cc2)C(C)C)C1=O ; *C1CC(=O)N(CCC(=O)NCC(=O)N[C@@H](CCC(=O)O)C(=O)Nc2ccc(S(*)(=O)=O)cc2)C1=O]

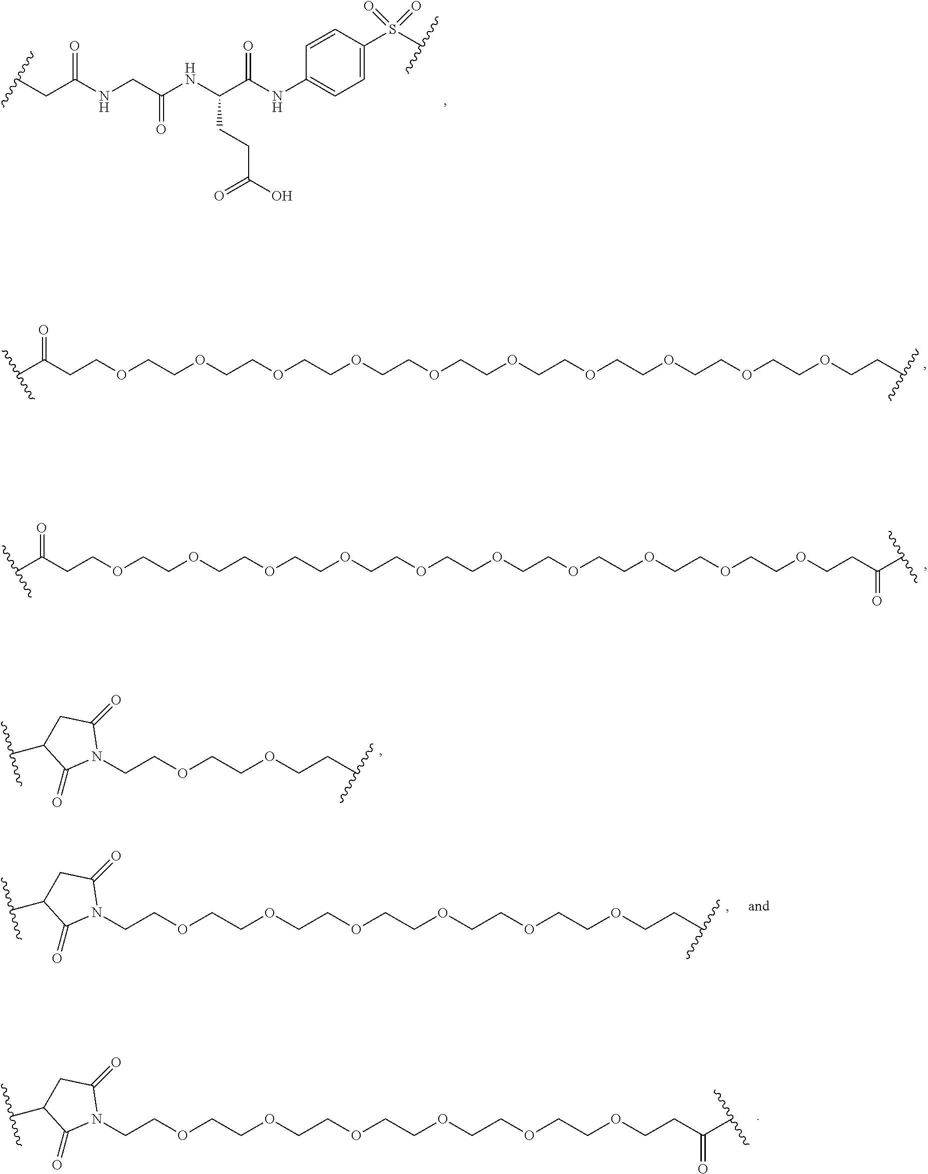 [SMILES: *CC(=O)NCC(=O)N[C@@H](CCC(=O)O)C(=O)Nc1ccc(S(*)(=O)=O)cc1 ; *CCOCCOCCOCCOCCOCCOCCOCCOCCOCCOCCC(*)=O ; *C(=O)CCOCCOCCOCCOCCOCCOCCOCCOCCOCCOCCC(*)=O ; *CCOCCOCCN1C(=O)CC(*)C1=O ; *CCOCCOCCOCCOCCOCCOCCN1C(=O)CC(*)C1=O ; *C(=O)CCOCCOCCOCCOCCOCCOCCN1C(=O)CC(*)C1=O]

For example, L is selected from

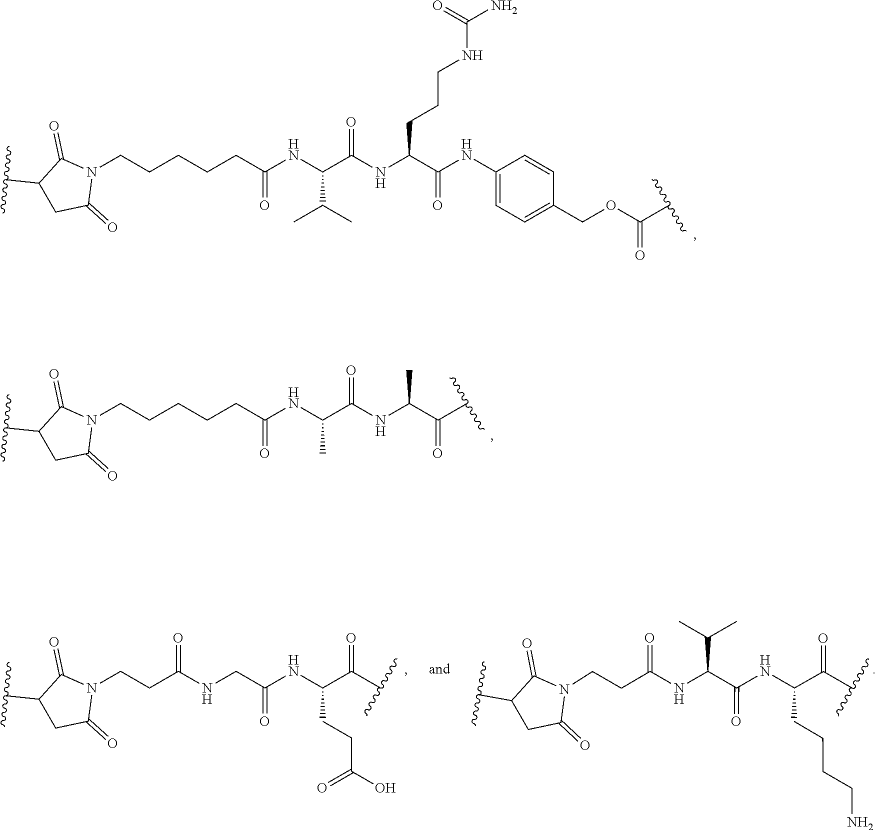 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)[C@H](C)NC(=O)[C@H](C)NC(=O)CCCCCN1C(=O)CC(*)C1=O ; *C(=O)[C@H](CCC(=O)O)NC(=O)CNC(=O)CCN1C(=O)CC(*)C1=O ; *C(=O)[C@H](CCCCN)NC(=O)[C@@H](NC(=O)CCN1C(=O)CC(*)C1=O)C(C)C]

In some embodiments, in the ligand-drug conjugate, or the isomer thereof, or the mixture thereof, or the pharmaceutically acceptable salt thereof described herein, the ligand-drug conjugate is represented by formulas (III-2C-1) and (III-2C-2):

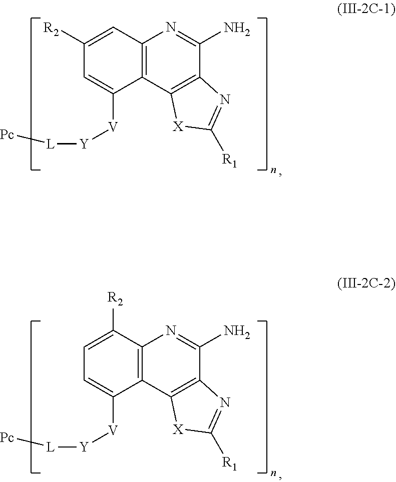

wherein
X is selected from —S— and —O—;
R₁ is selected from C₁-C₆ alkyl optionally substituted with 1, 2, or 3 R₁₁, each R₁₁ being independently selected from: hydrogen, chlorine, bromine, iodine, hydroxy, amino, C₁-C₃ alkyl, and C₃-C₆ cycloalkyl; the methylene units of R₁ are each independently unreplaced or replaced by a group selected from: —O—, —N(CH₃)—, and —NH—;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂₋₁; each R₂₋₁ is independently selected from: hydrogen, halogen, hydroxy, and amino;
V is selected from C₁-C₈ alkylene, preferably C₁-C₆ alkylene, wherein one or more carbon atoms in the C₁-C₈ alkylene and the C₁-C₆ alkylene may be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
Y is selected from —N(R_{Y1})—;
R_{Y1} is selected from hydrogen and C₁-C₆ alkyl;
L is -L₃-L₂-L_{1c}- or -Tr-L₃-L₂-L_{1c}-;
Tr is selected from:

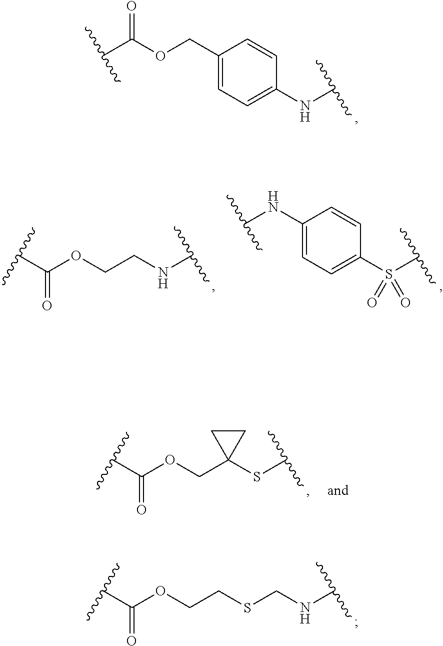 [SMILES: *Nc1ccc(COC(*)=O)cc1 ; *NCCOC(*)=O ; *Nc1ccc(S(*)(=O)=O)cc1 ; *SC1(COC(*)=O)CC1 ; *NCSCCOC(*)=O]

L_{1c} is selected from

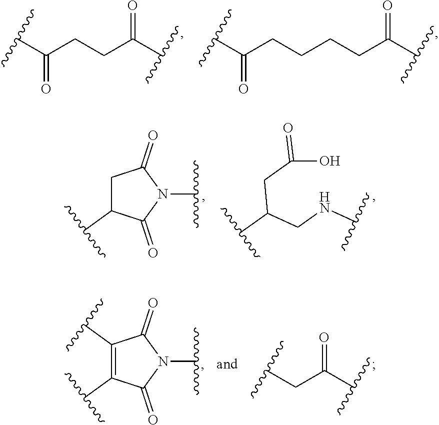 [SMILES: *C(=O)CCC(*)=O ; *C(=O)CCCCC(*)=O ; *C1CC(=O)N(*)C1=O ; *NCC(*)CC(=O)O ; *C1=C(*)C(=O)N(*)C1=O ; *CC(*)=O]

L₂ is selected from

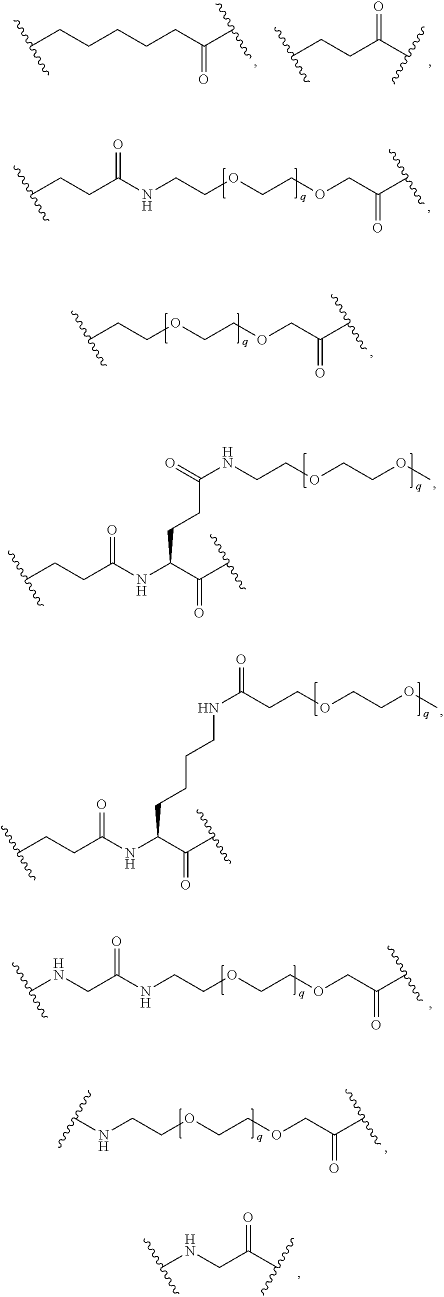

and a single bond, wherein q is an integer from 1 to 20;
L₃ is selected from the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-;
n is an integer or a decimal from 1 to 10;
Pc is a ligand.

In certain preferred embodiments of the present disclosure, in the structures represented by formulas (III-2C-1) and (III-2C-2), R₁, R₂, V, Y, L, n, and Pc are as described in any one of the embodiments of (II-2C) of the present application.

In some embodiments, in the ligand-drug conjugate, or the isomer thereof, or the mixture thereof, or the pharmaceutically acceptable salt thereof of the present disclosure, the ligand-drug conjugate is selected from the following structural formulas:

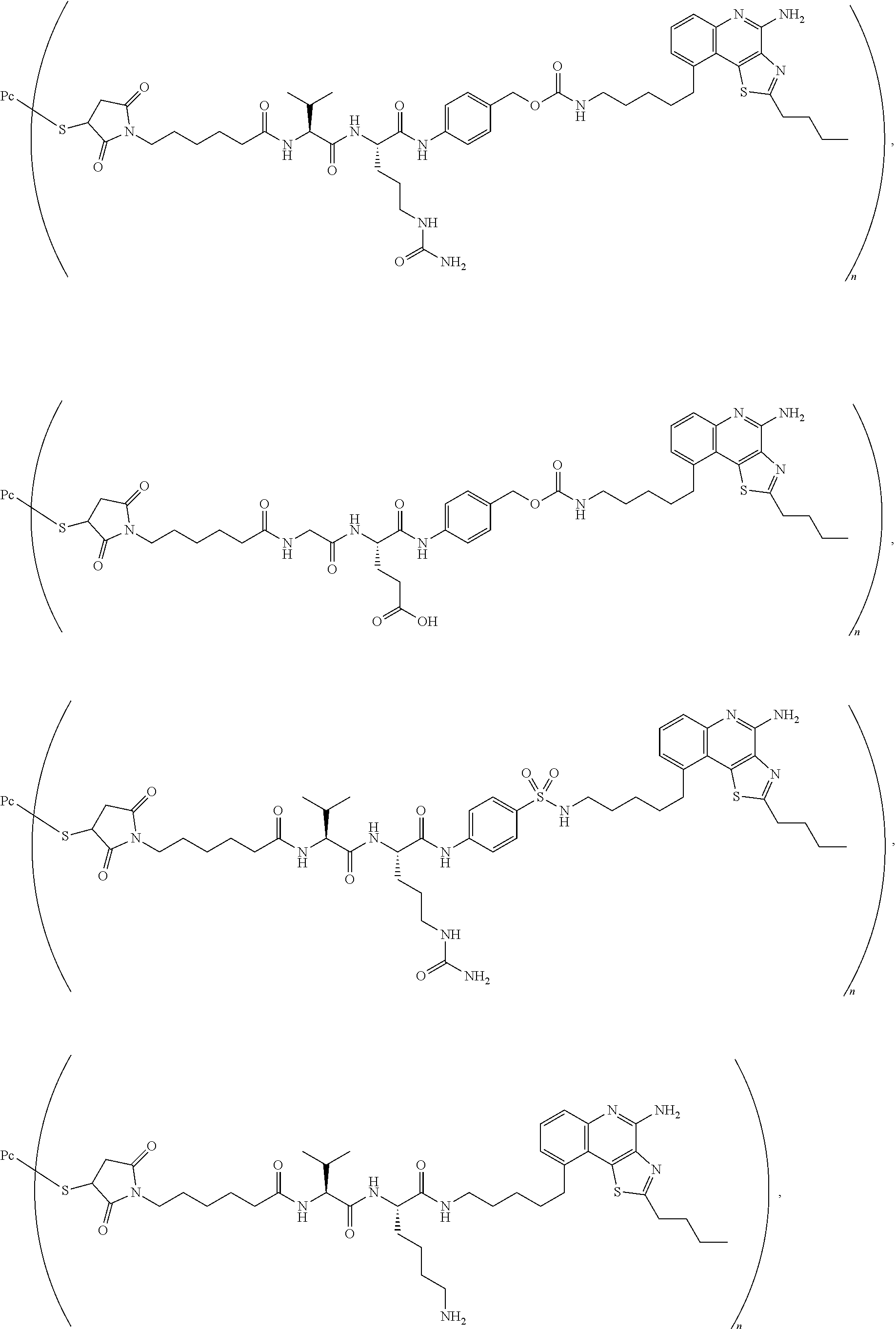

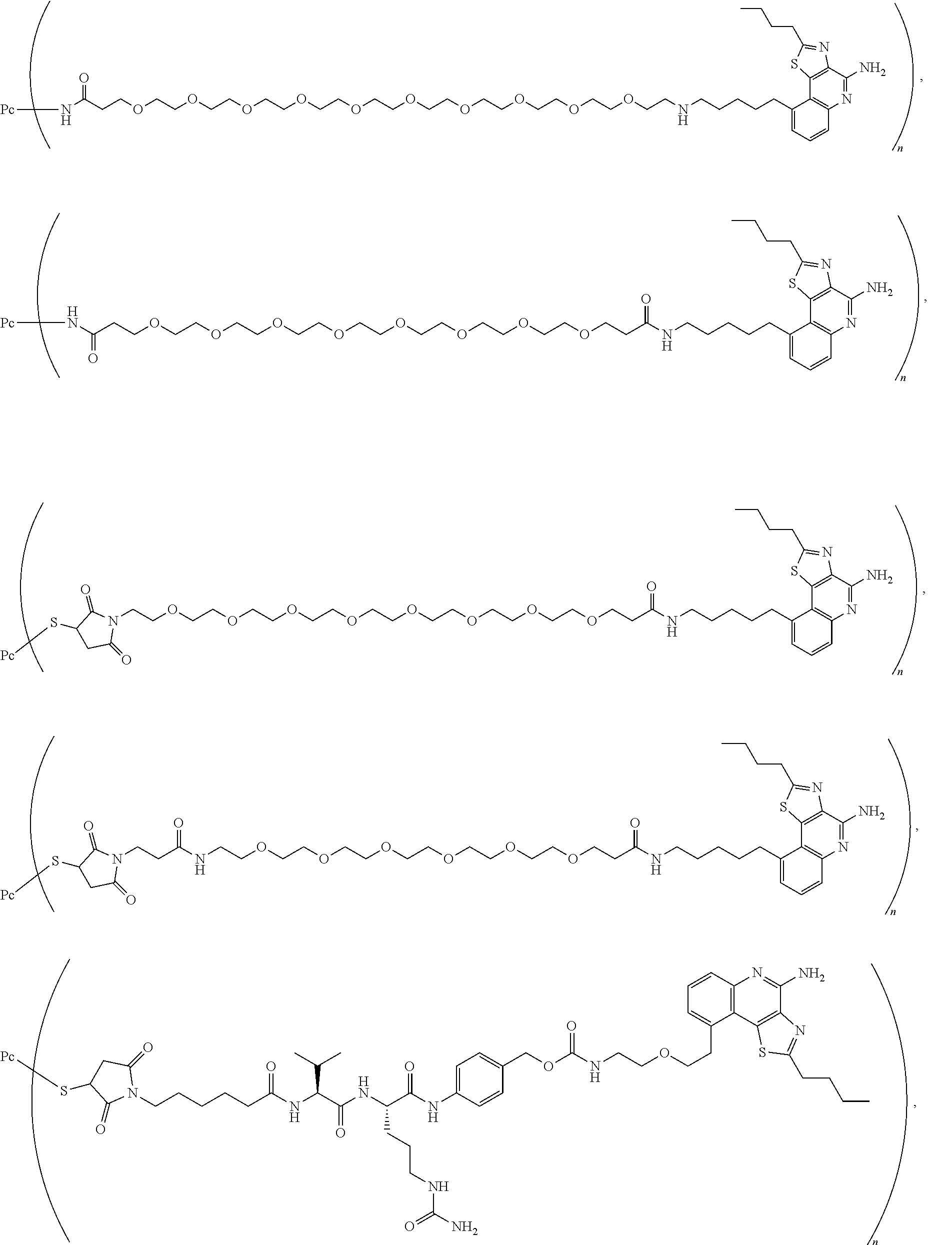

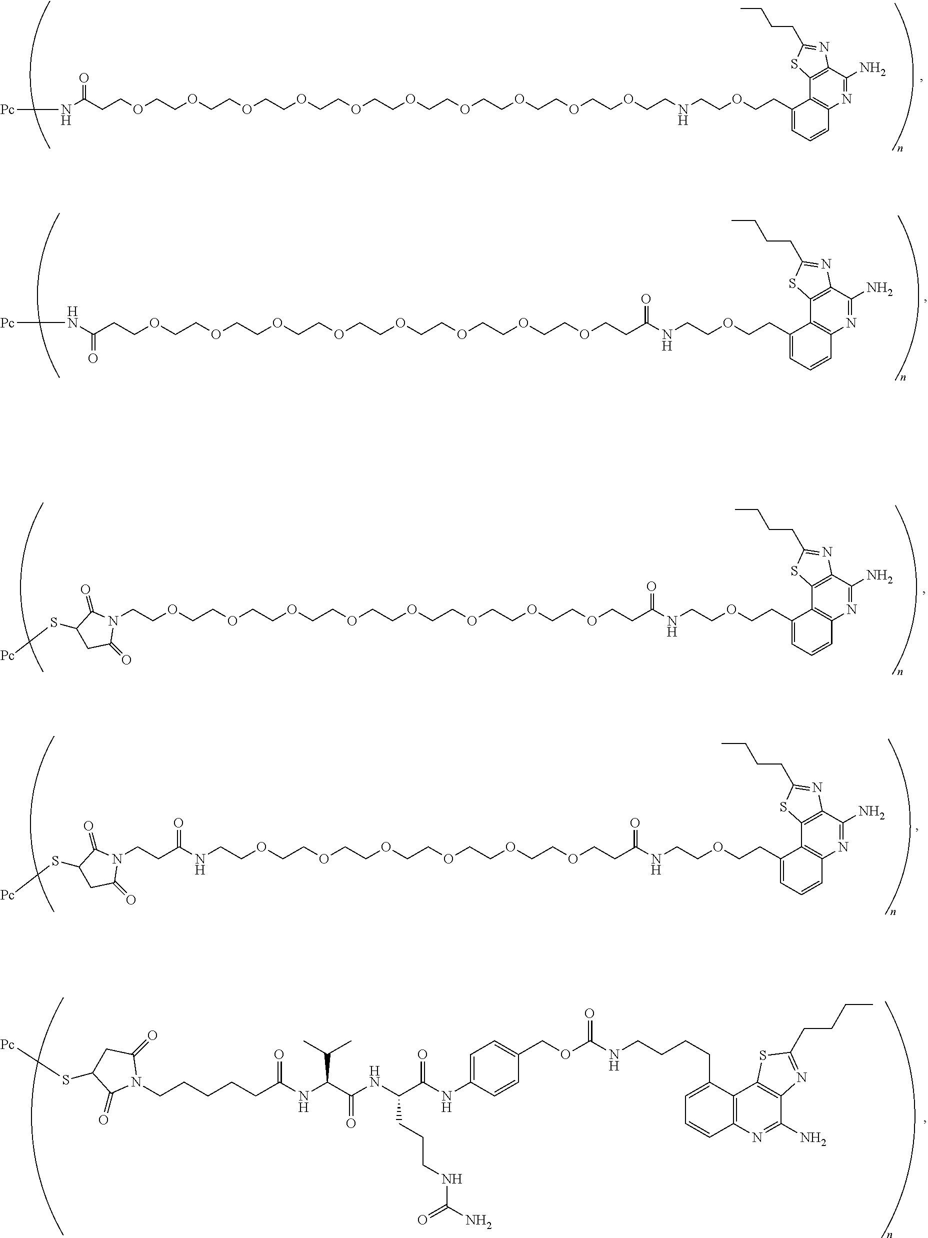

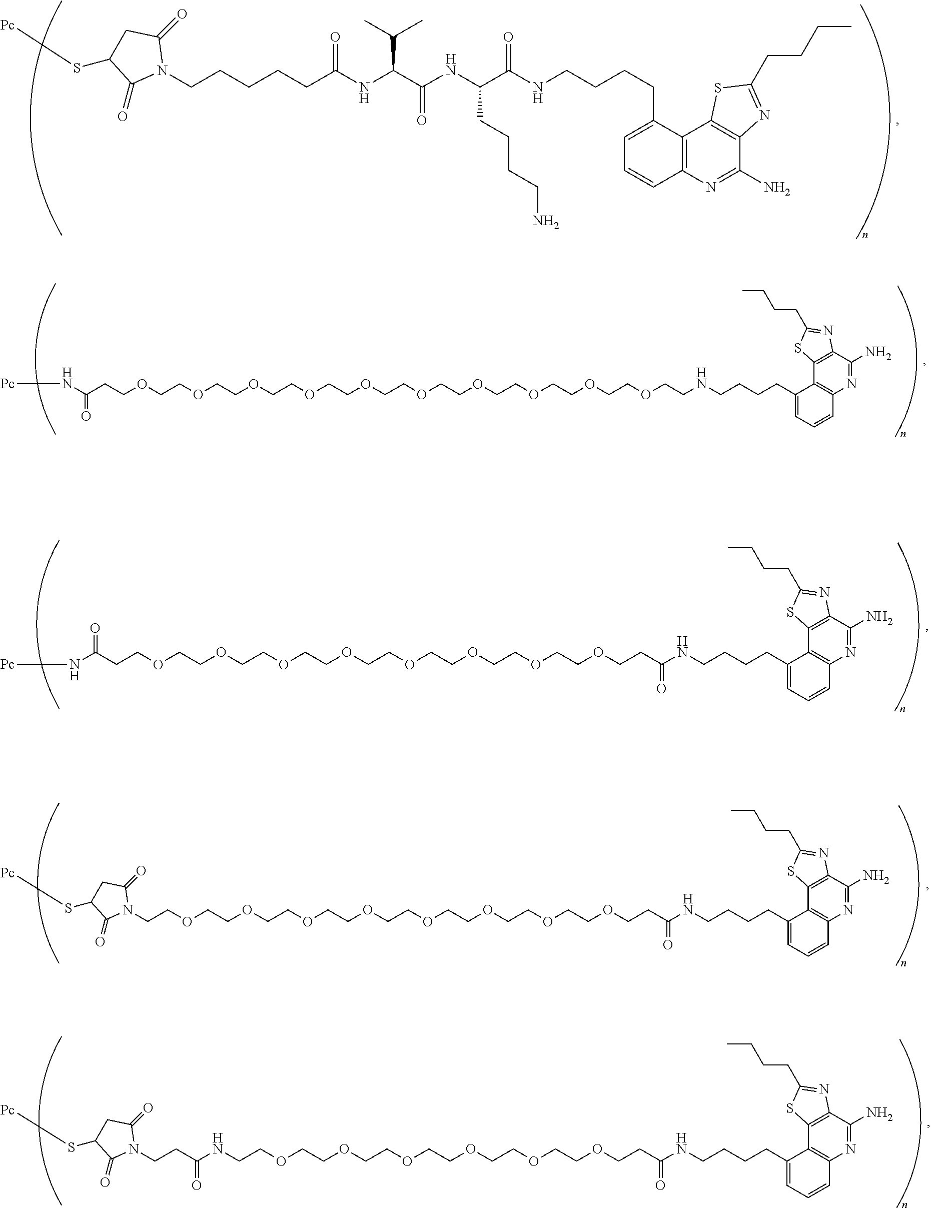

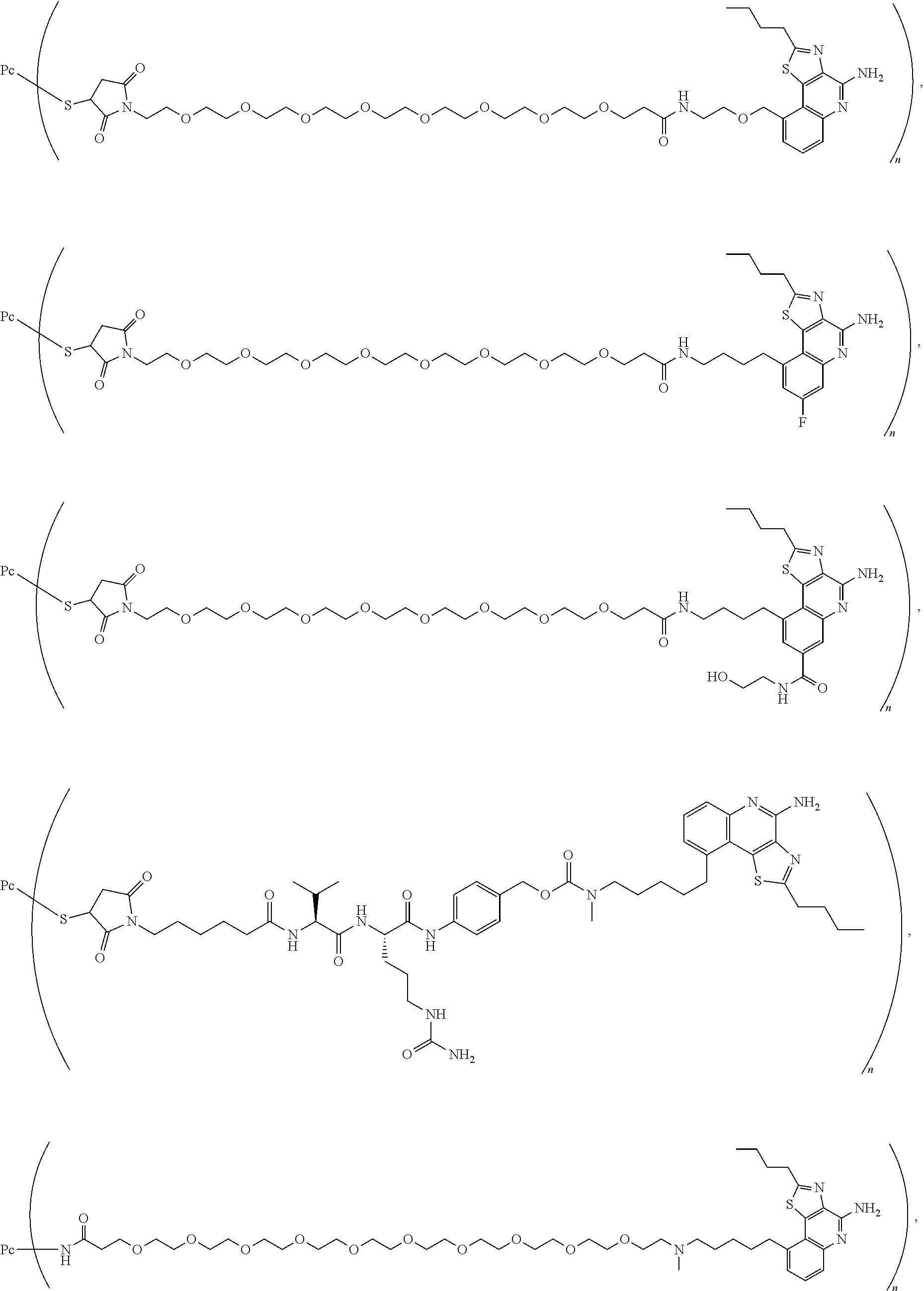

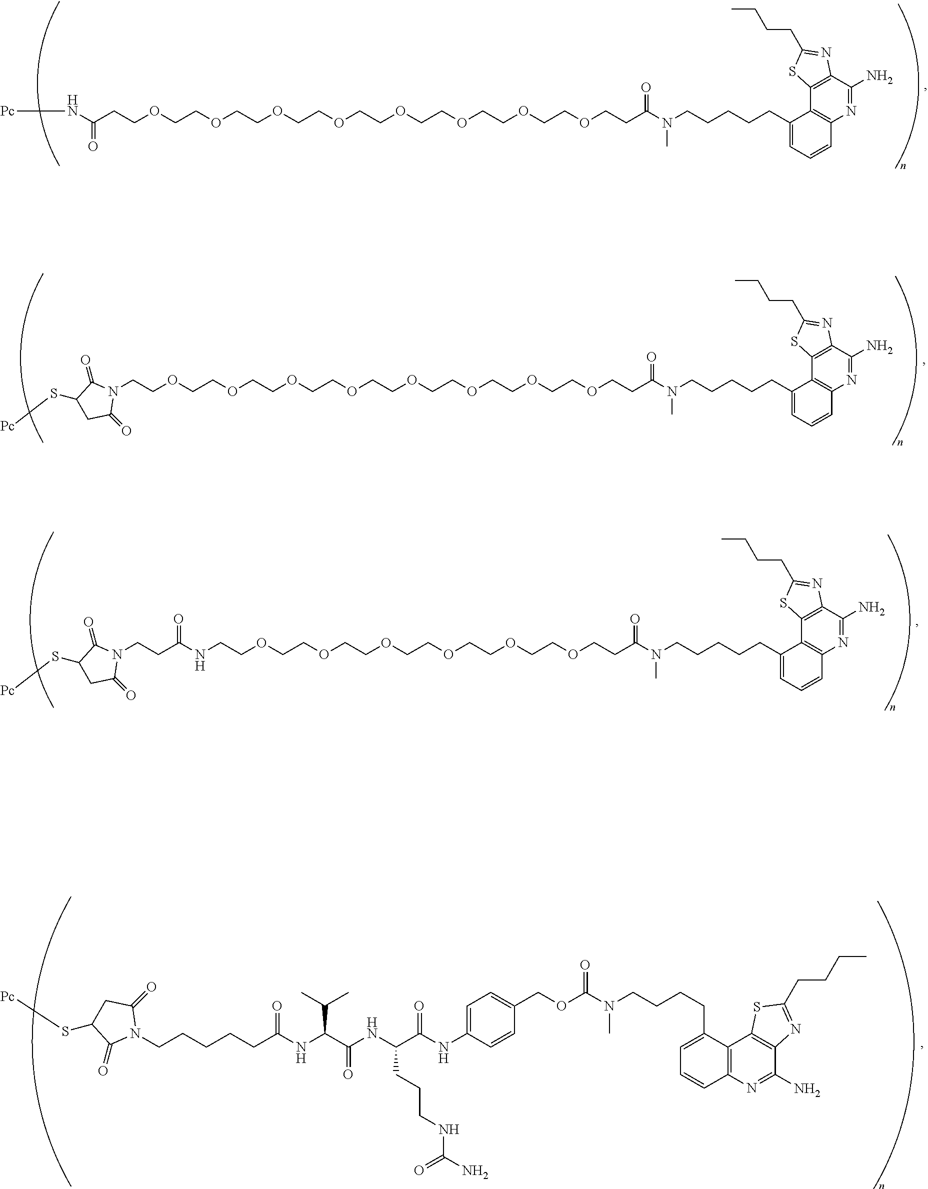

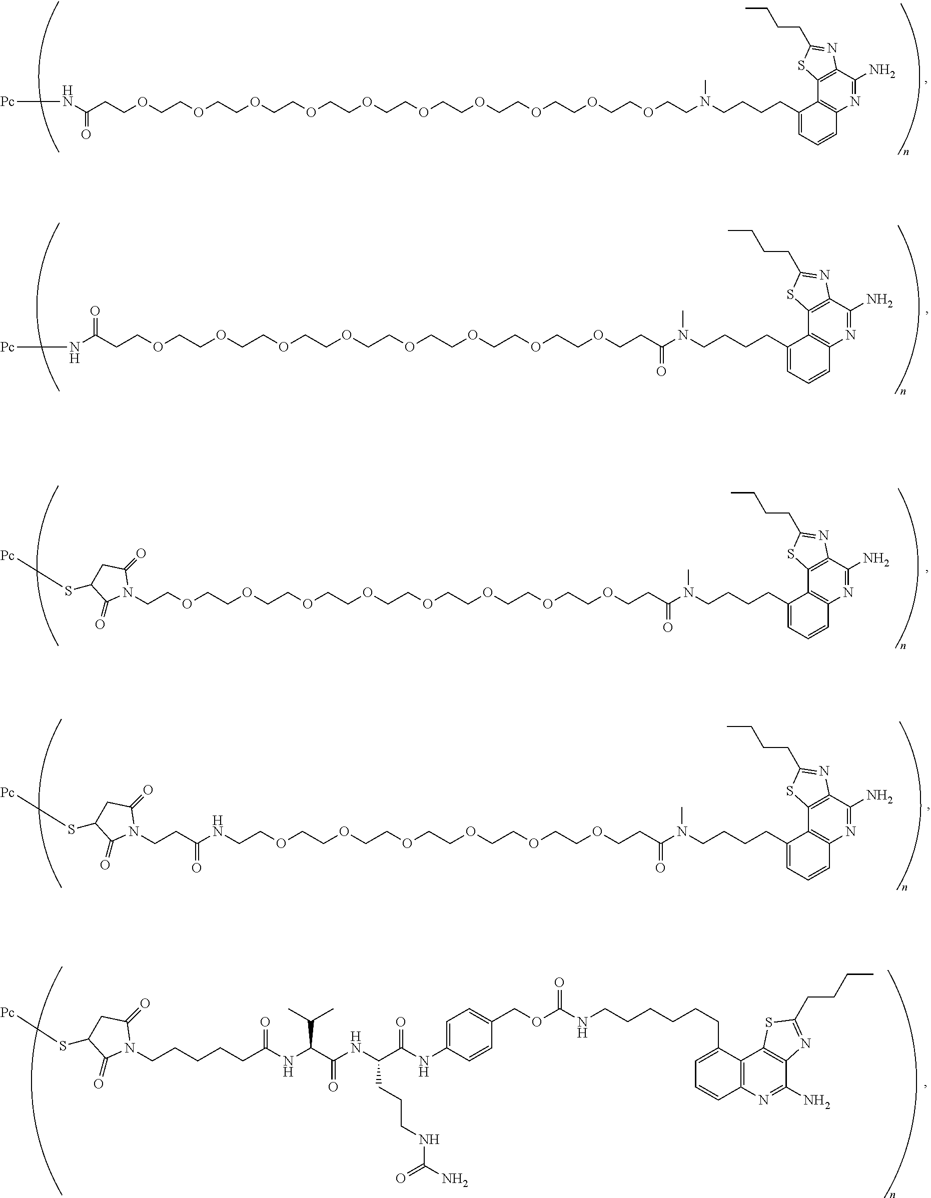

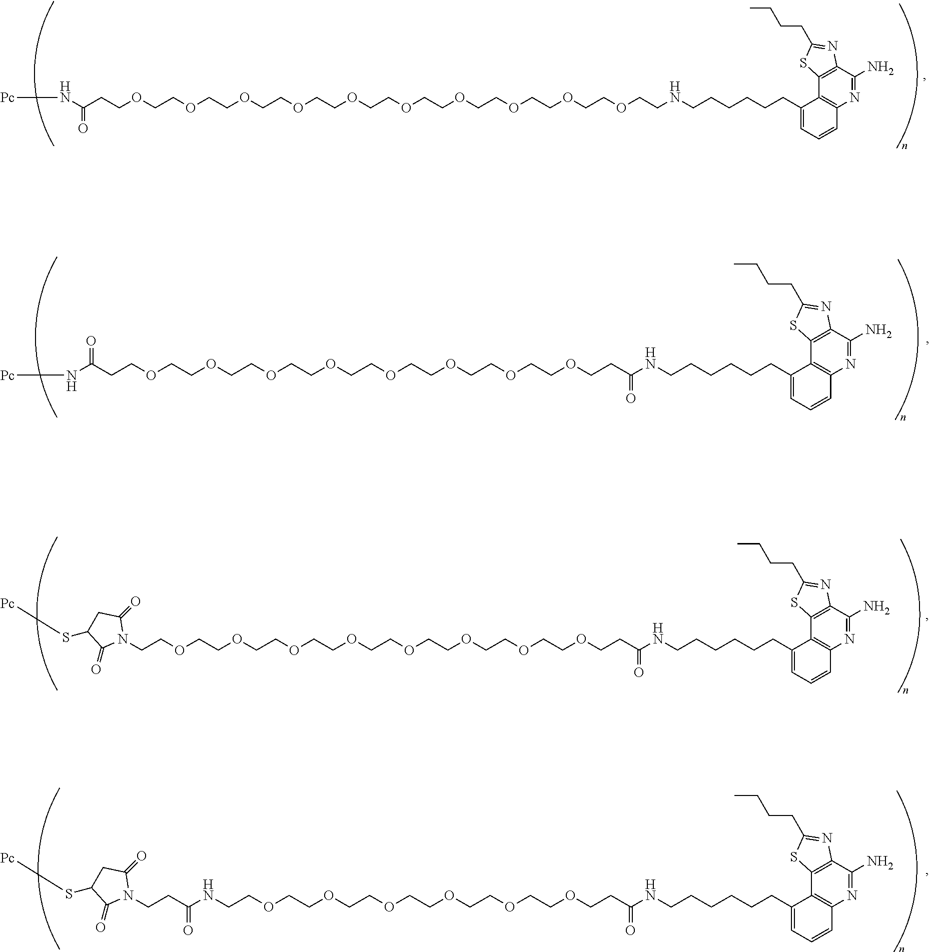

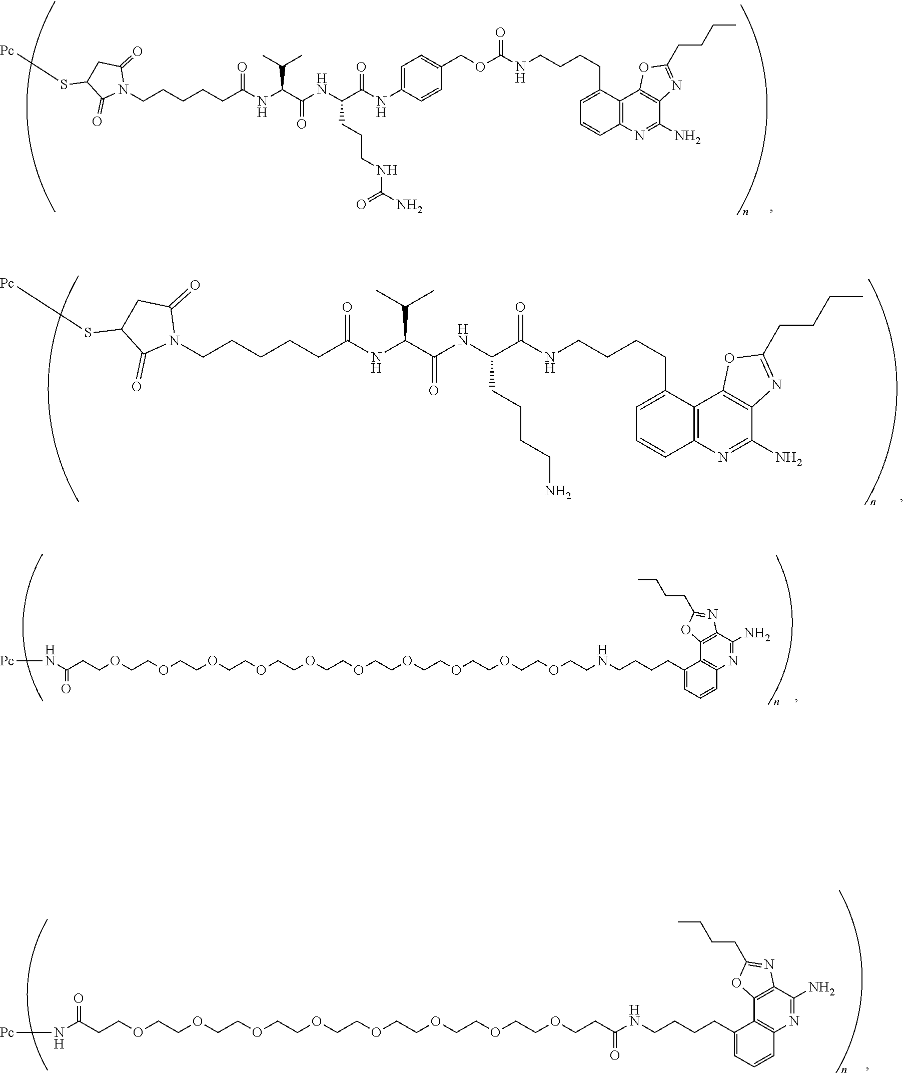

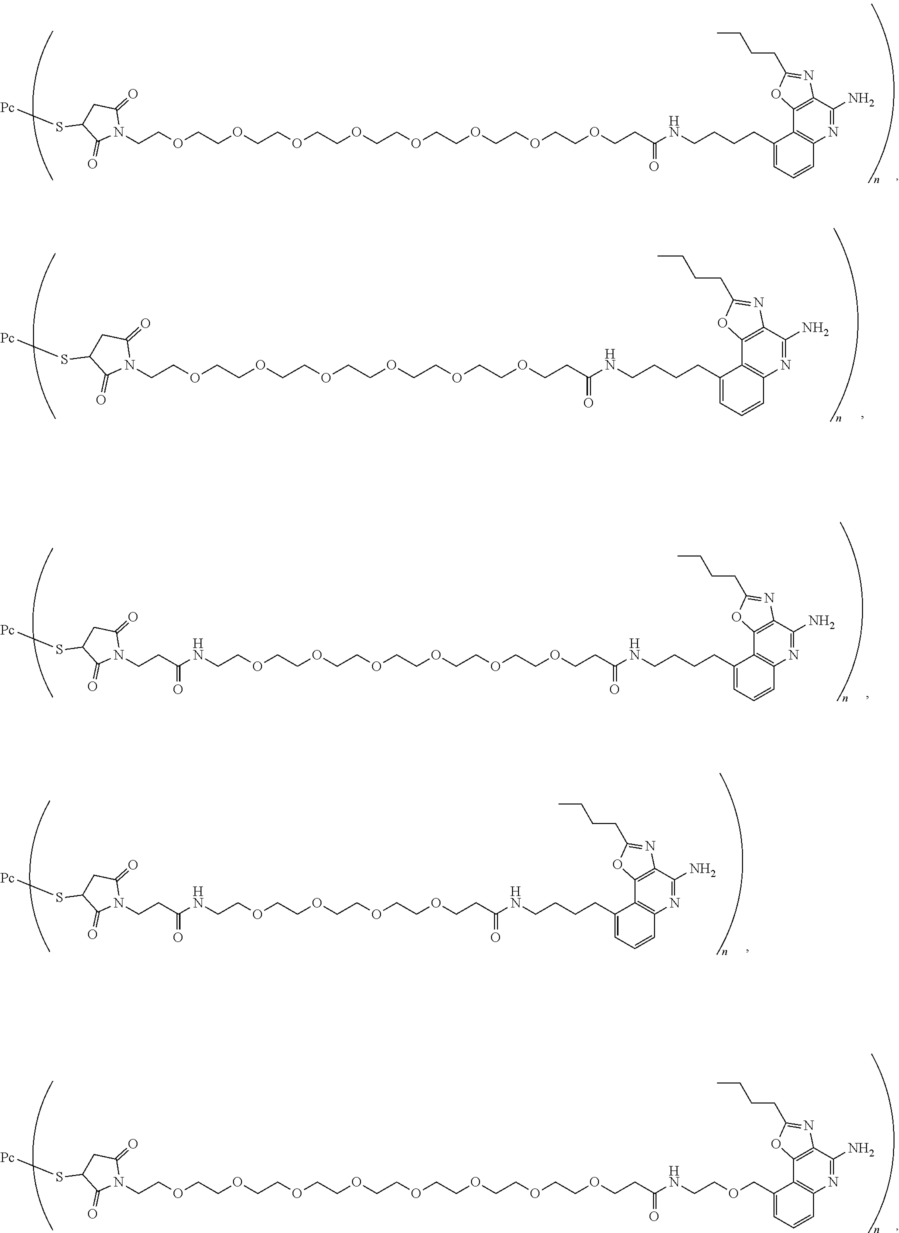

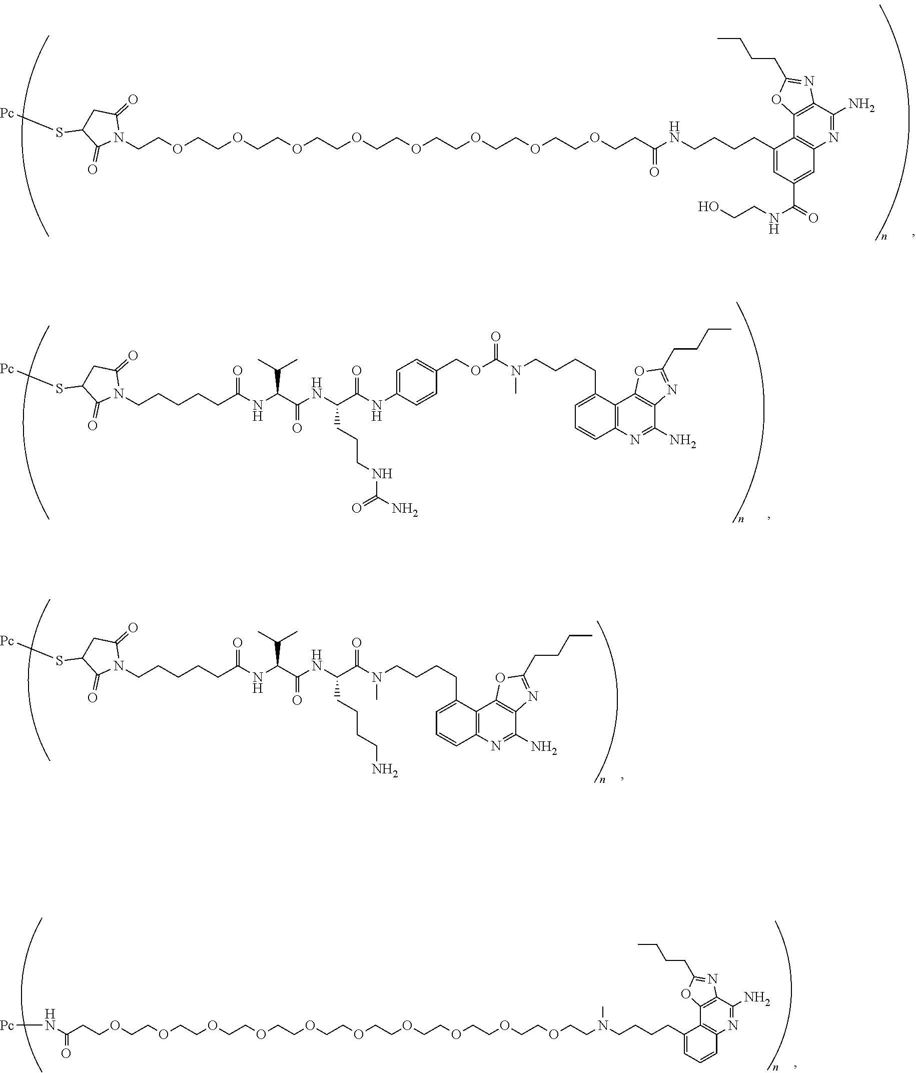

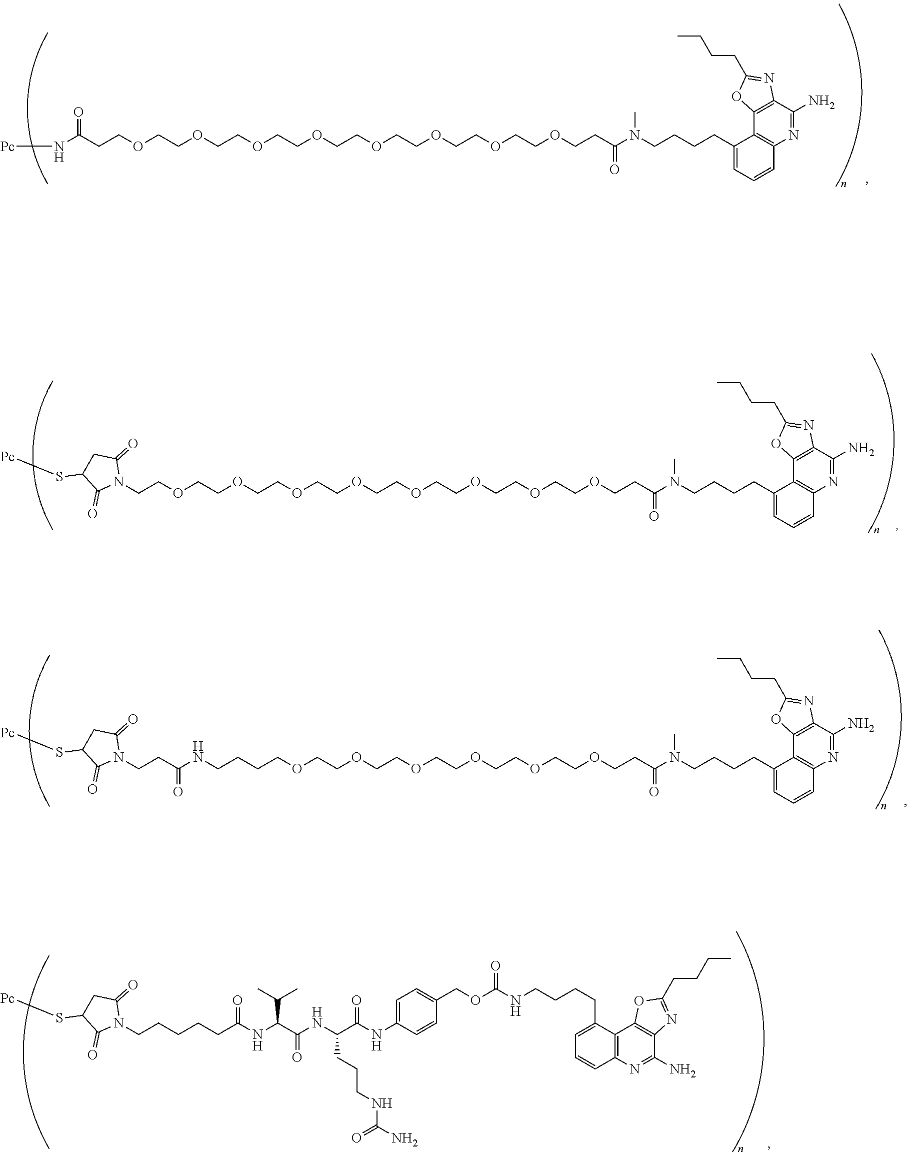

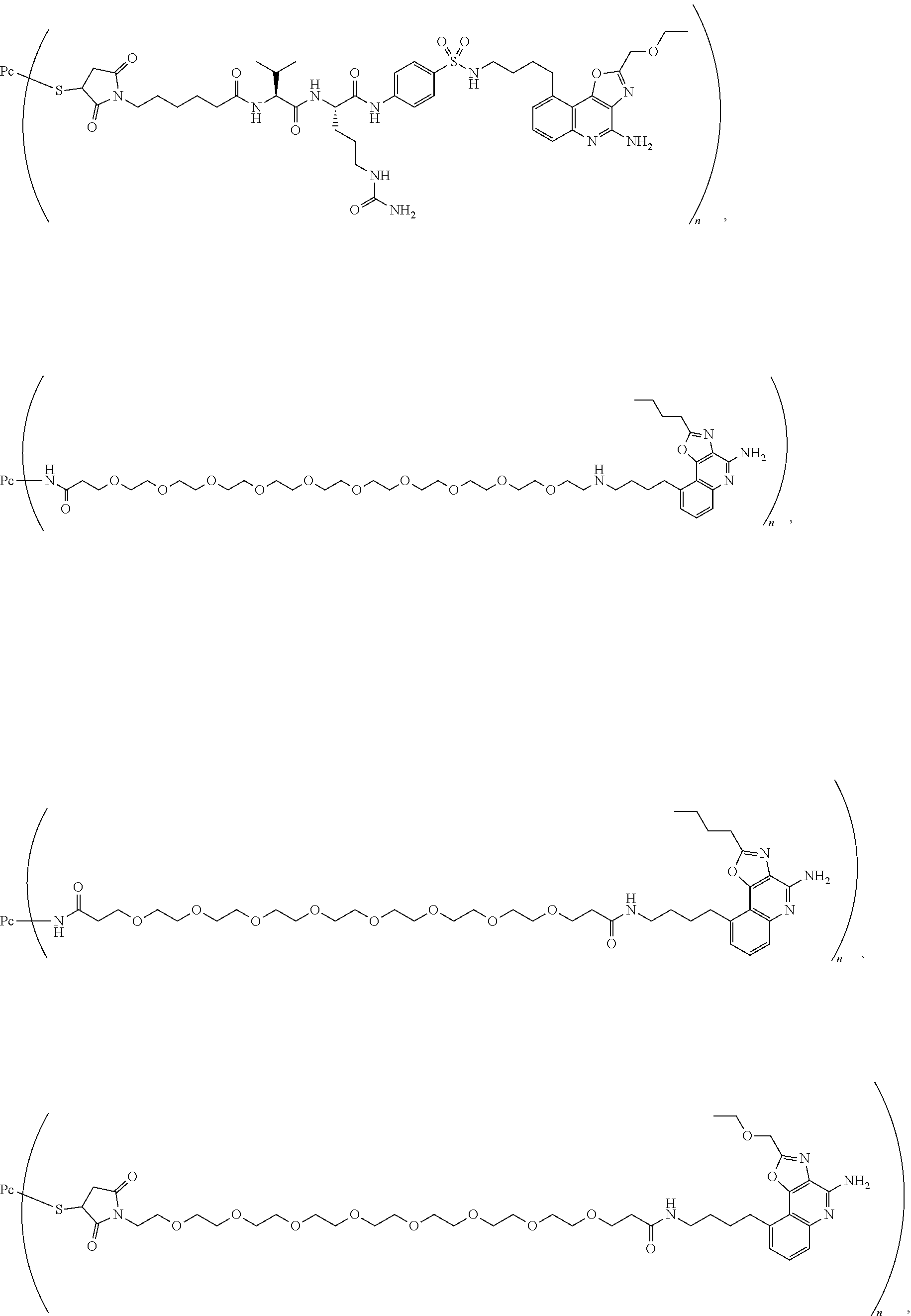

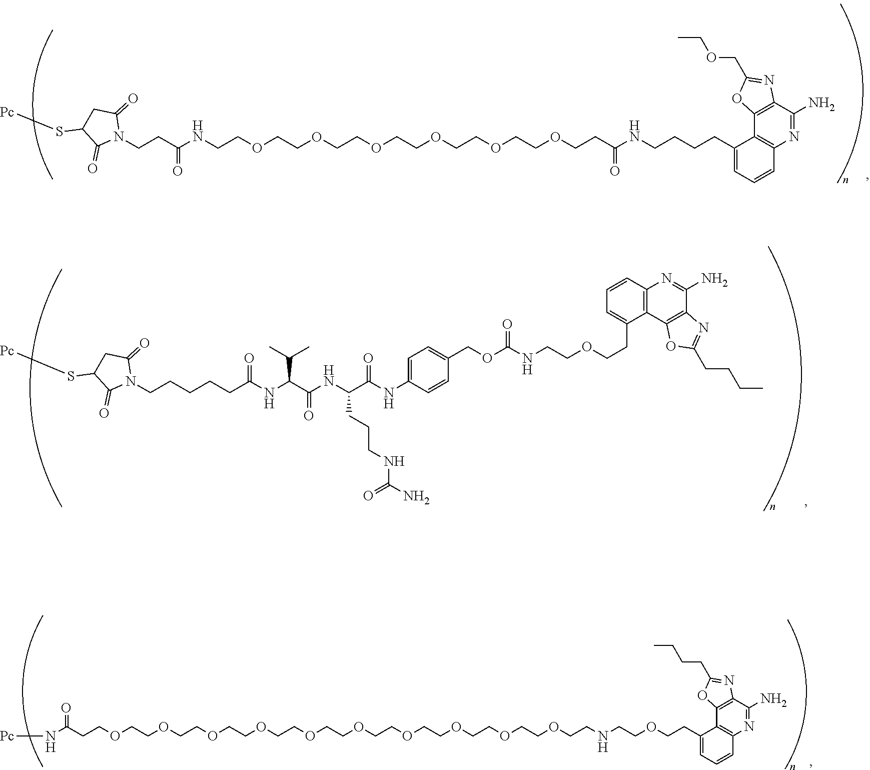

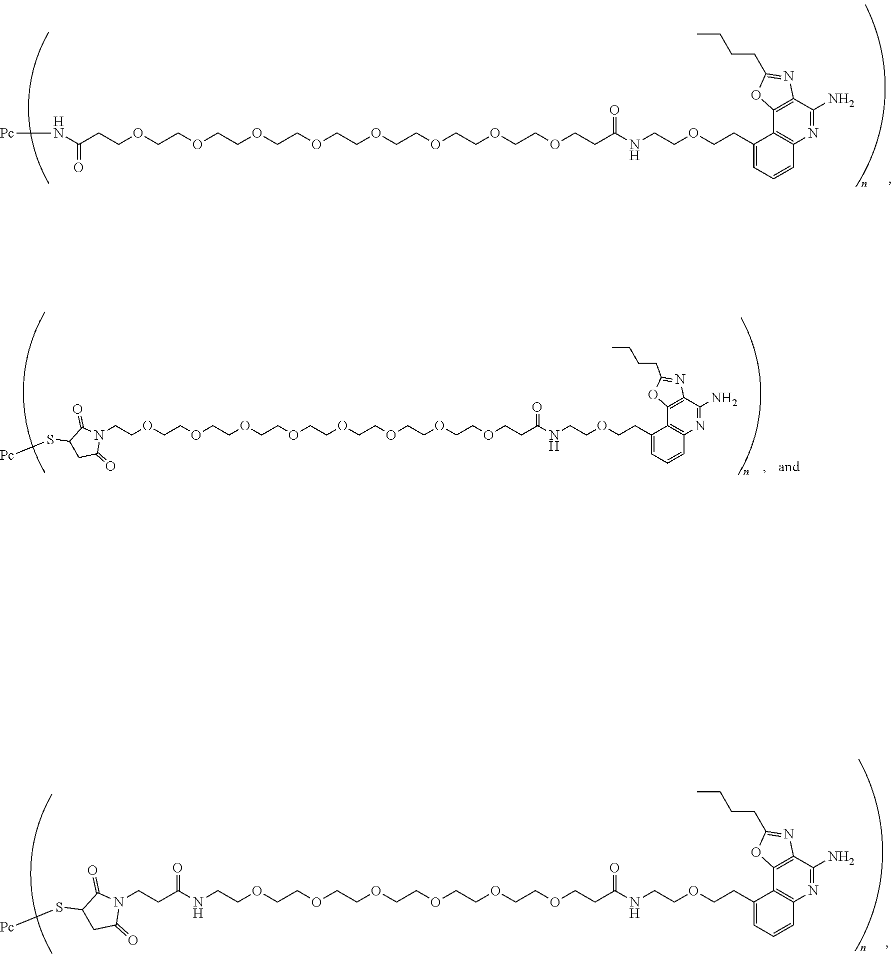

wherein
n is an integer or a decimal from 1 to 10;
Pc is a ligand.

In some embodiments, in the ligand-drug conjugate, or the isomer thereof, or the mixture thereof, or the pharmaceutically acceptable salt thereof described herein, the ligand-drug conjugate is selected from the following structural formulas:

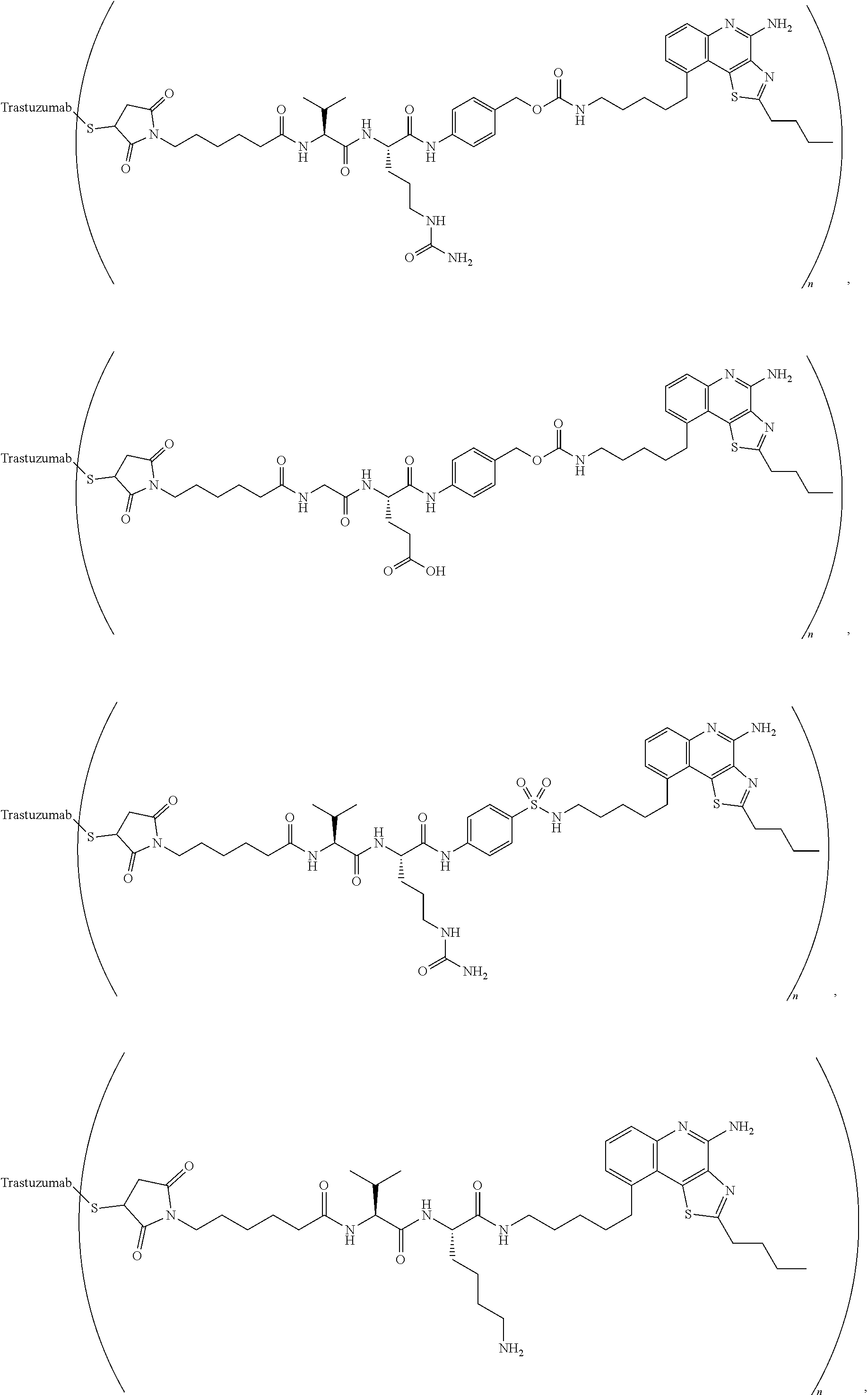

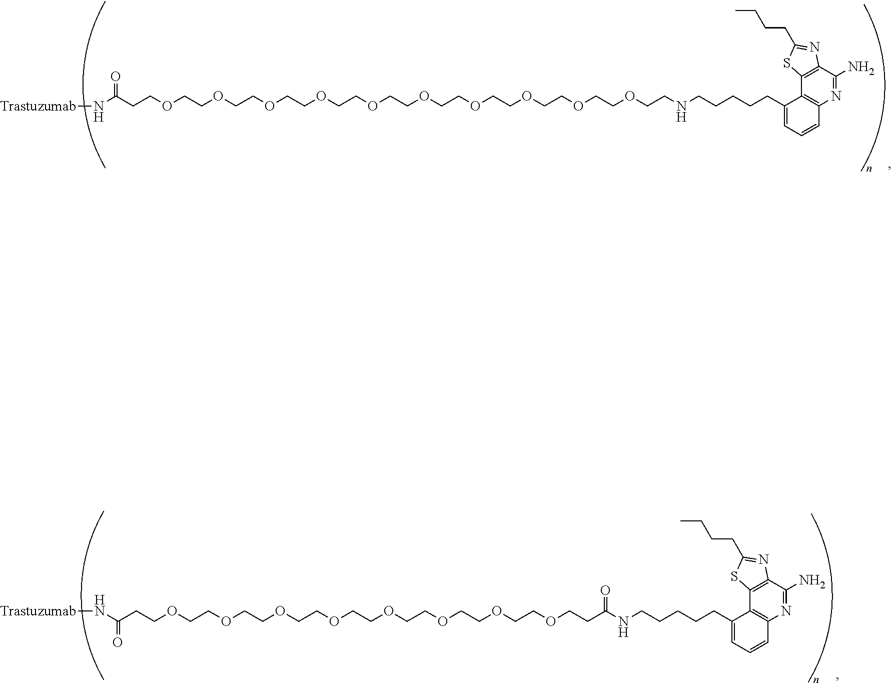

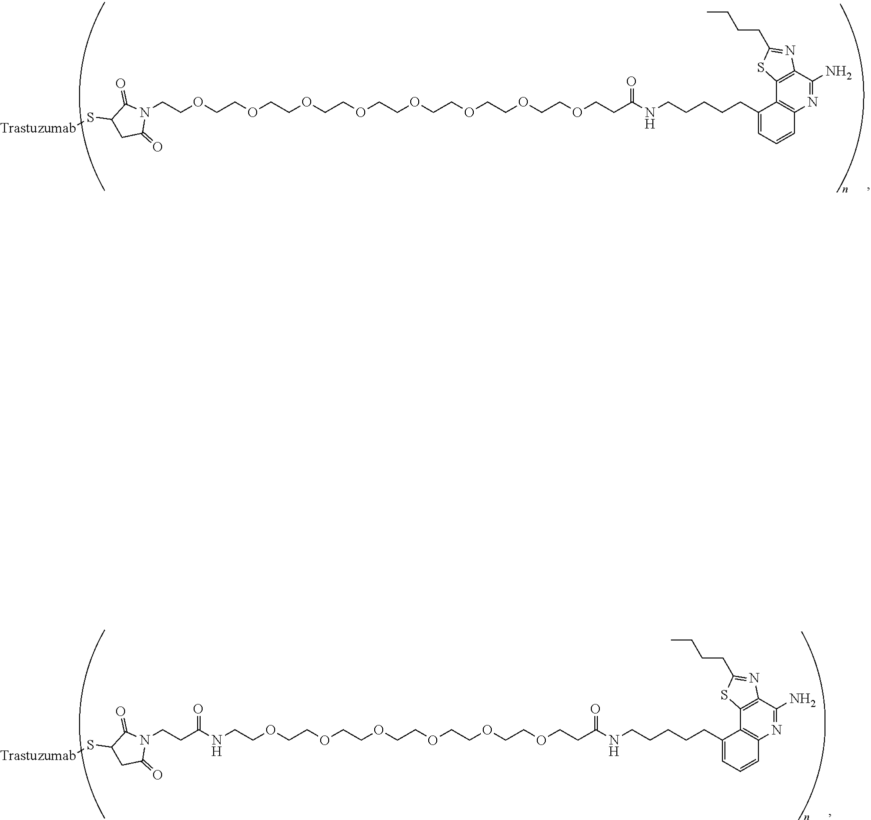

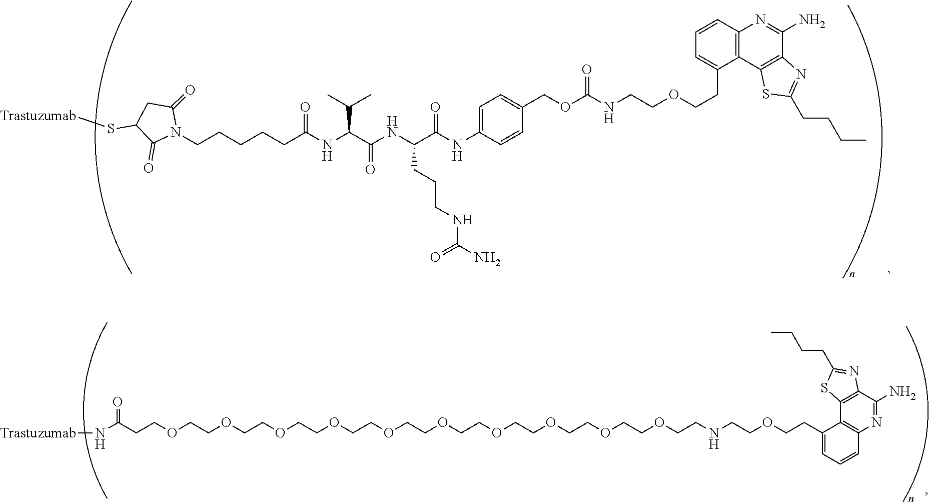

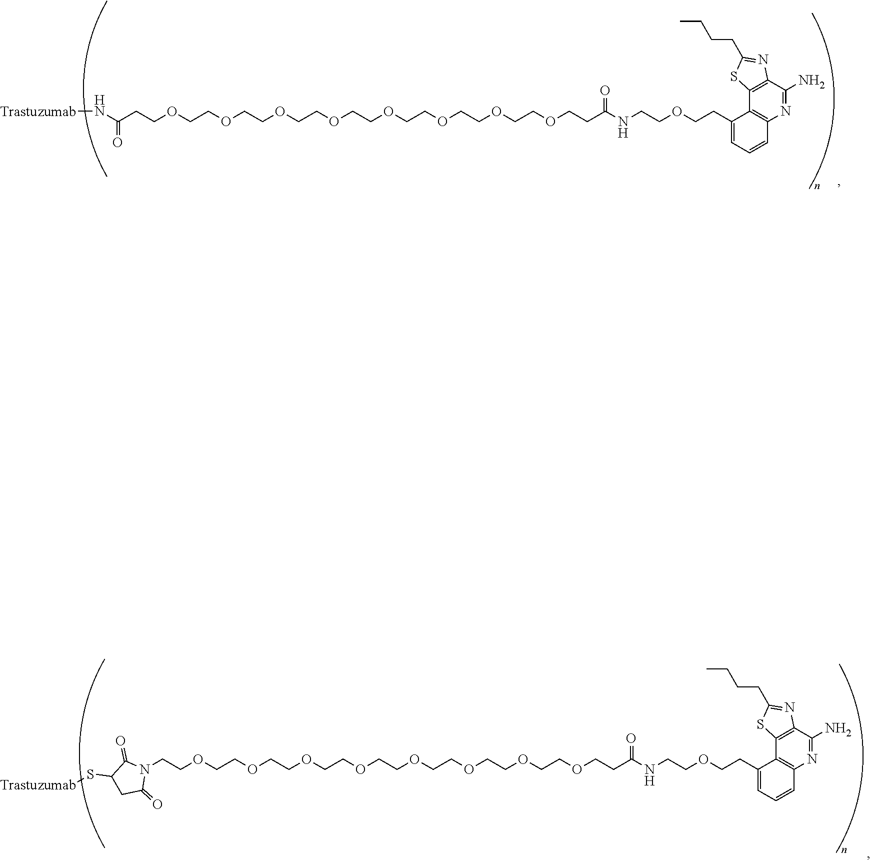

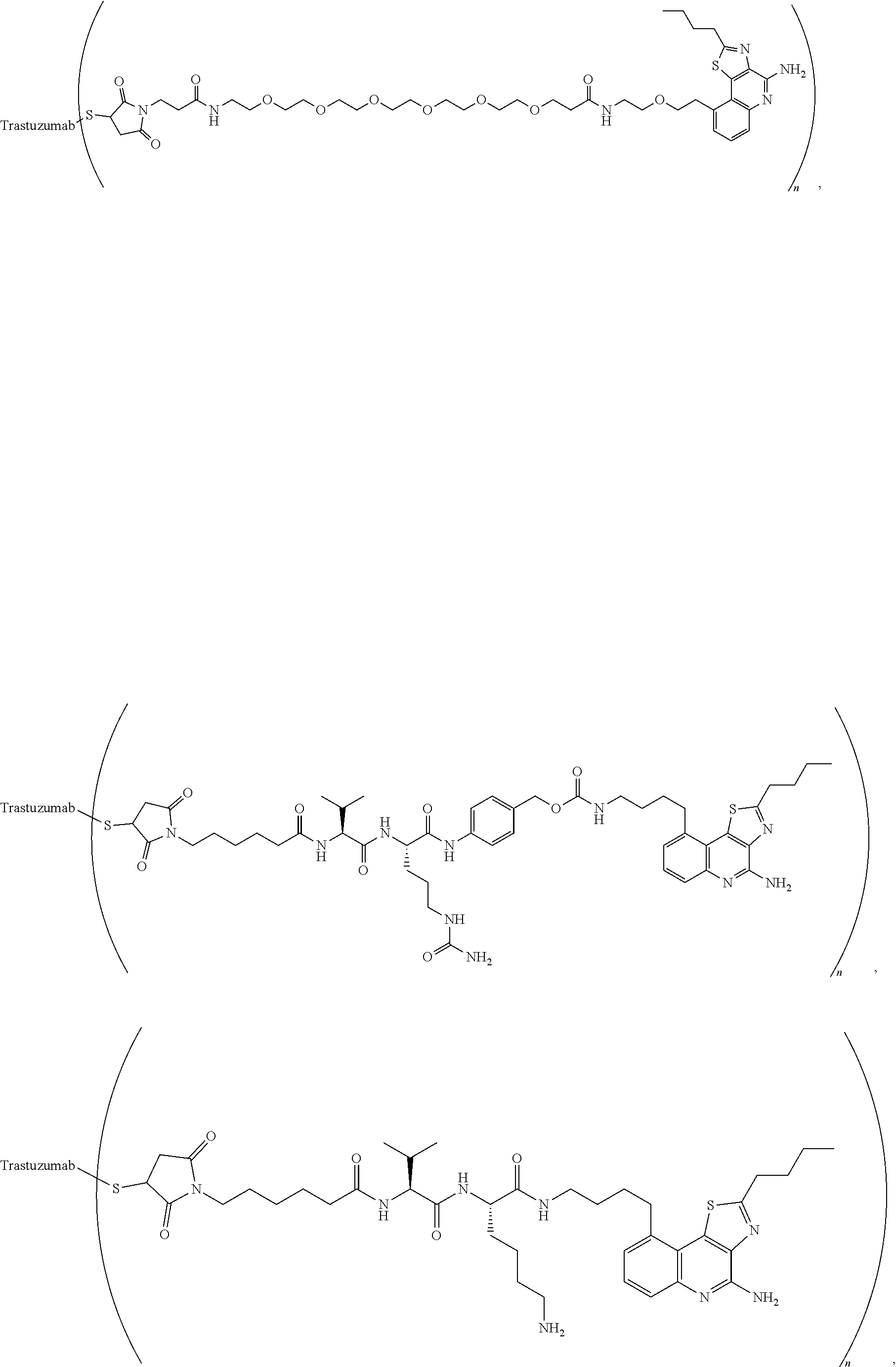

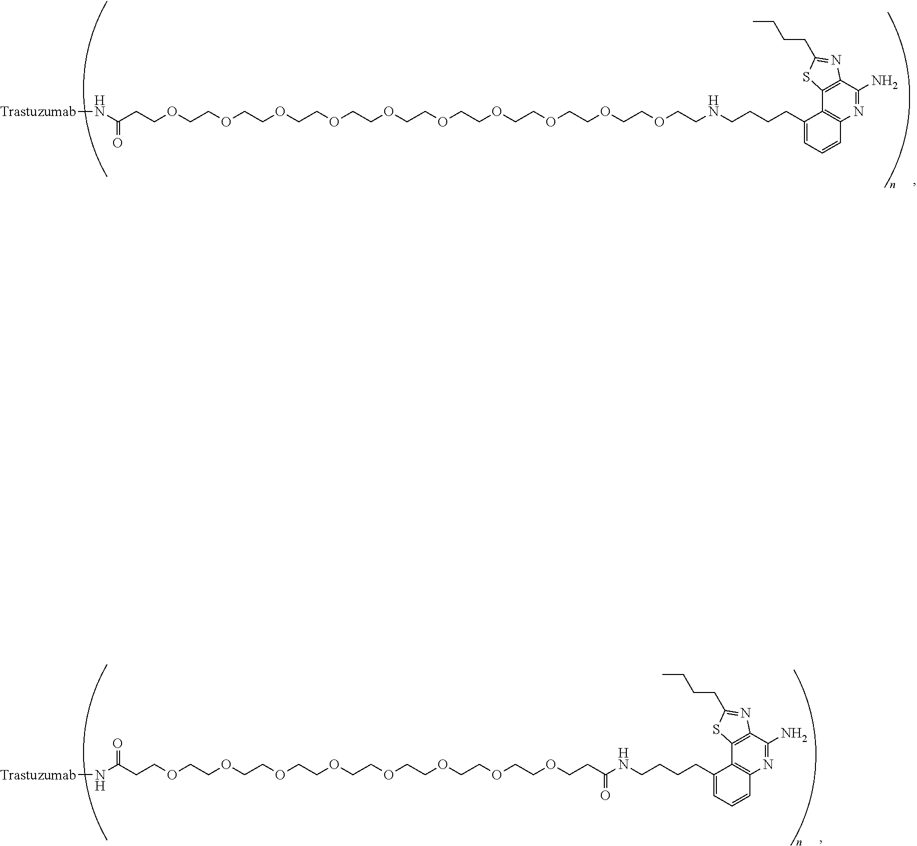

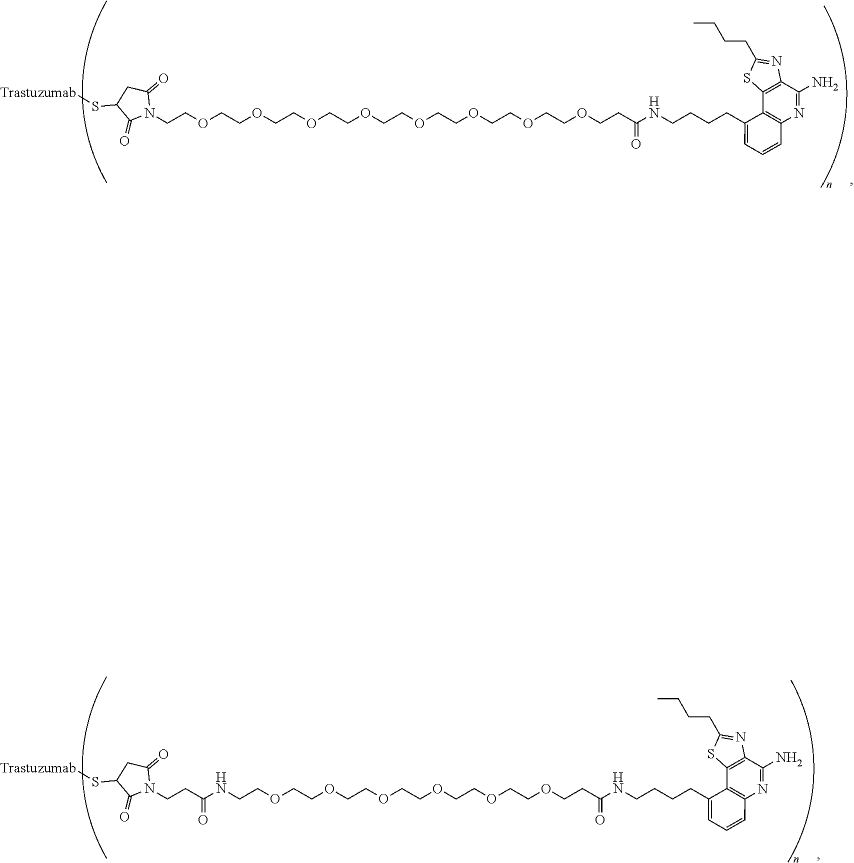

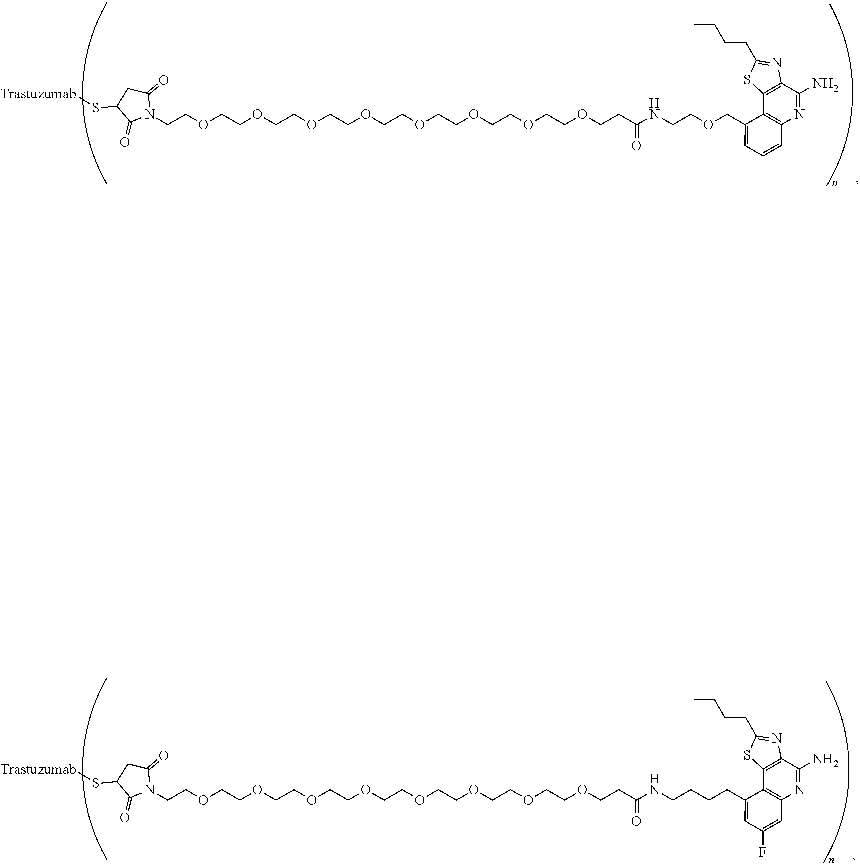

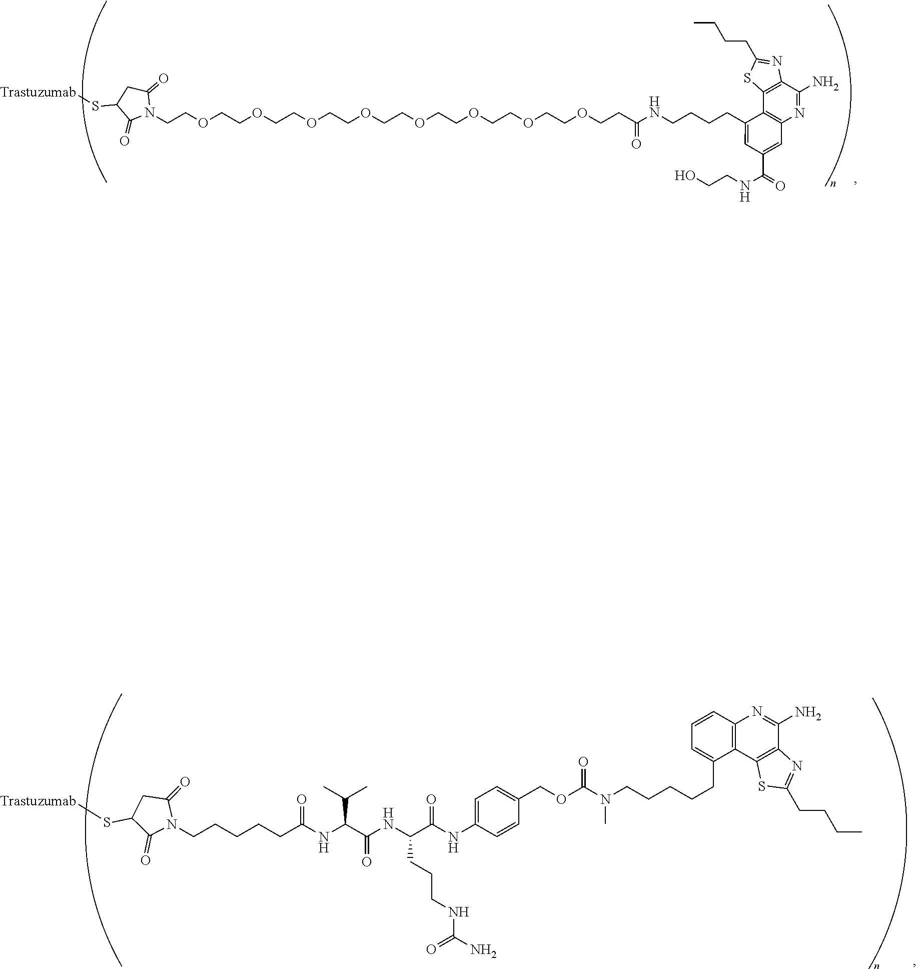

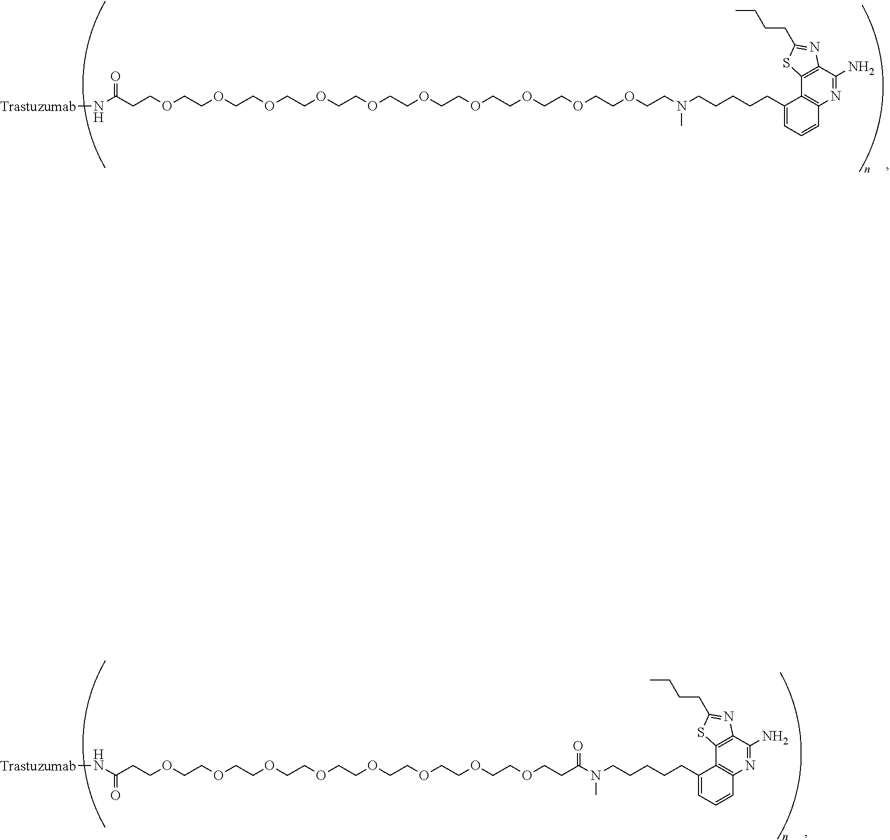

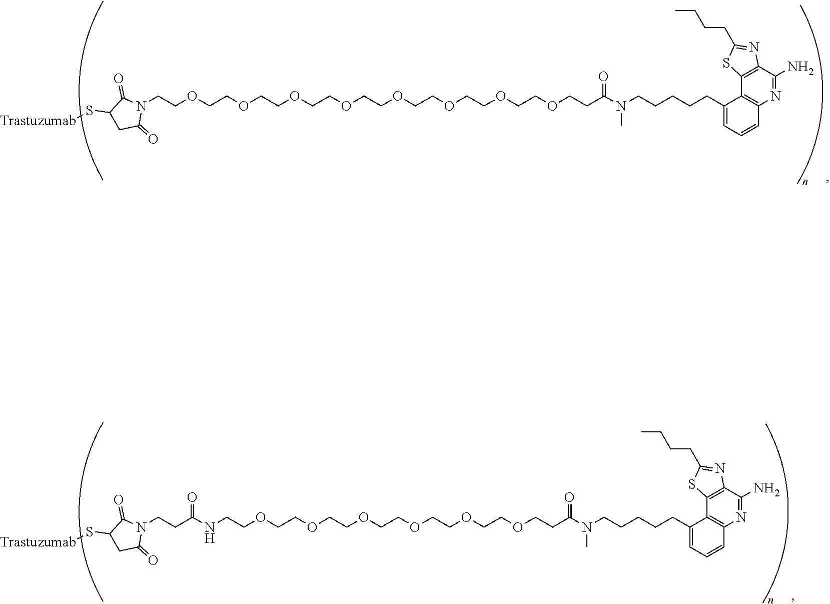

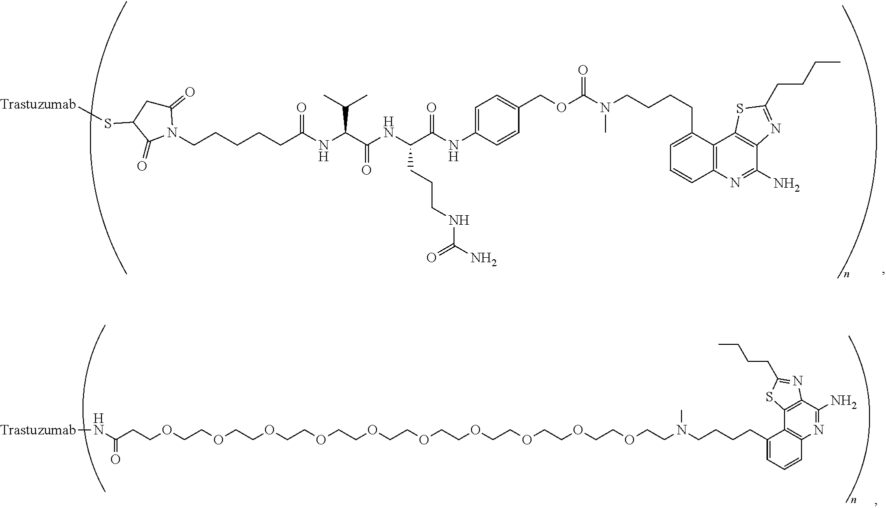

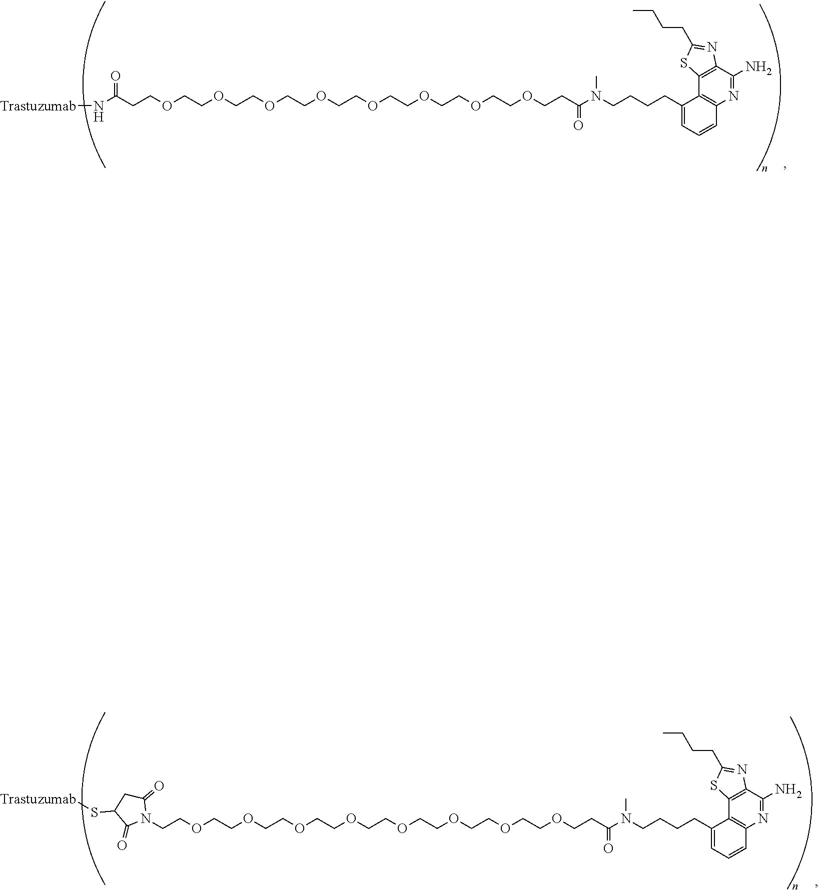

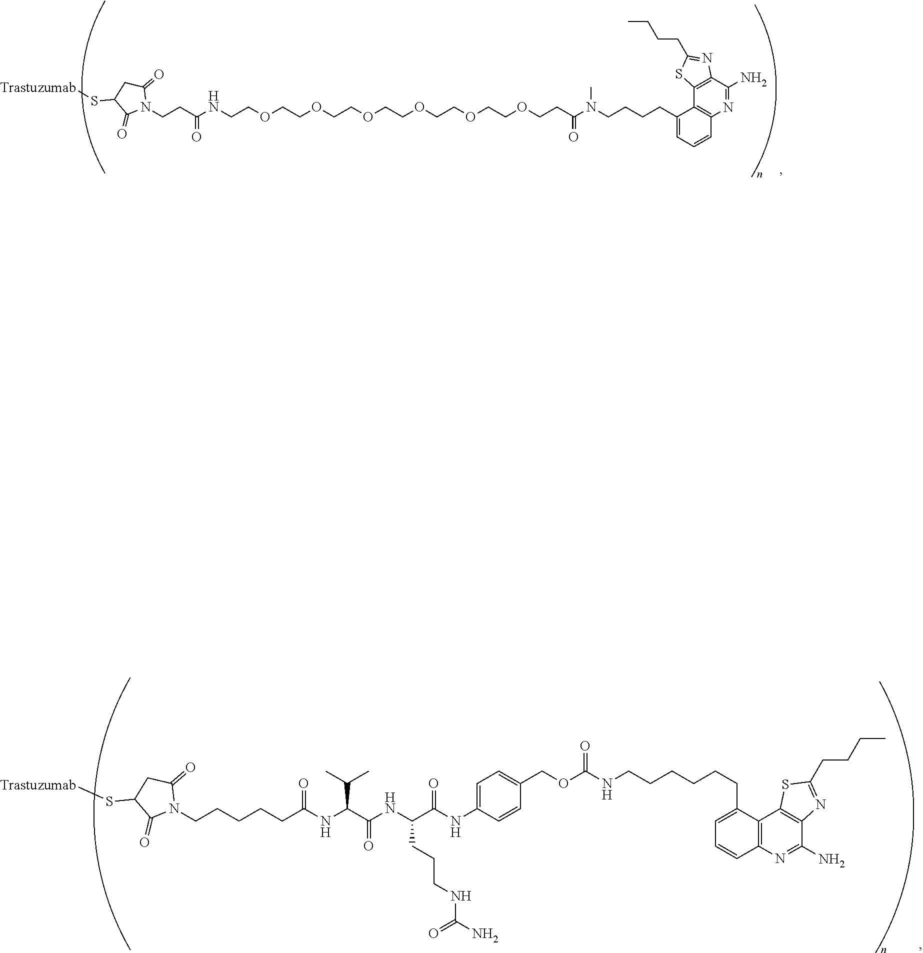

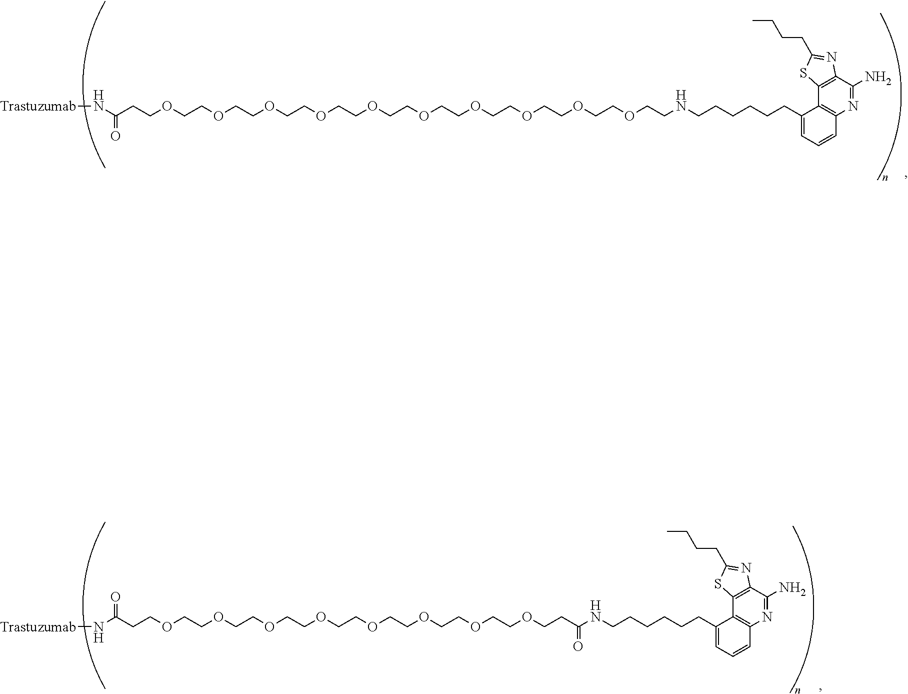

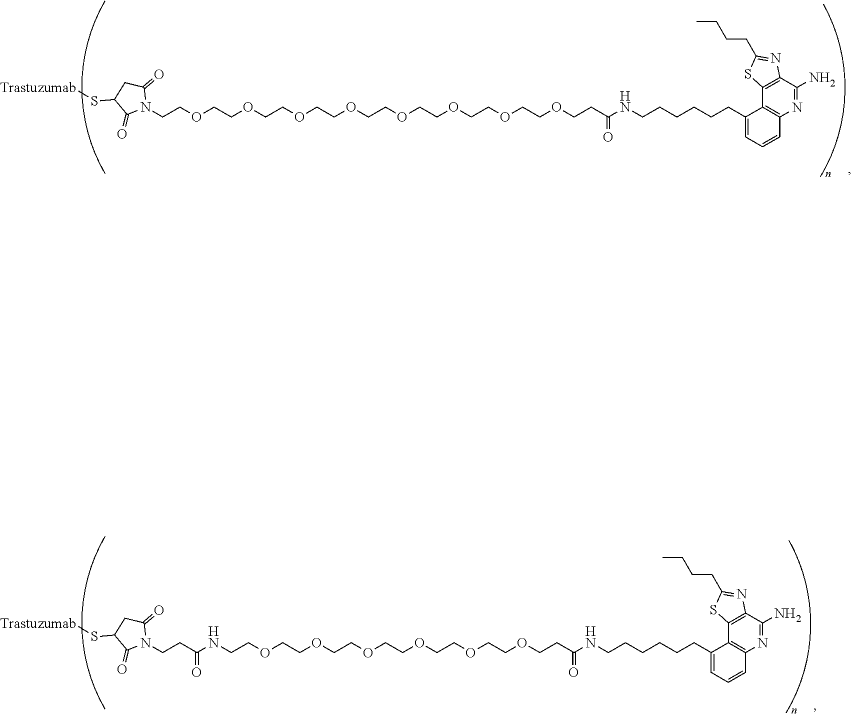

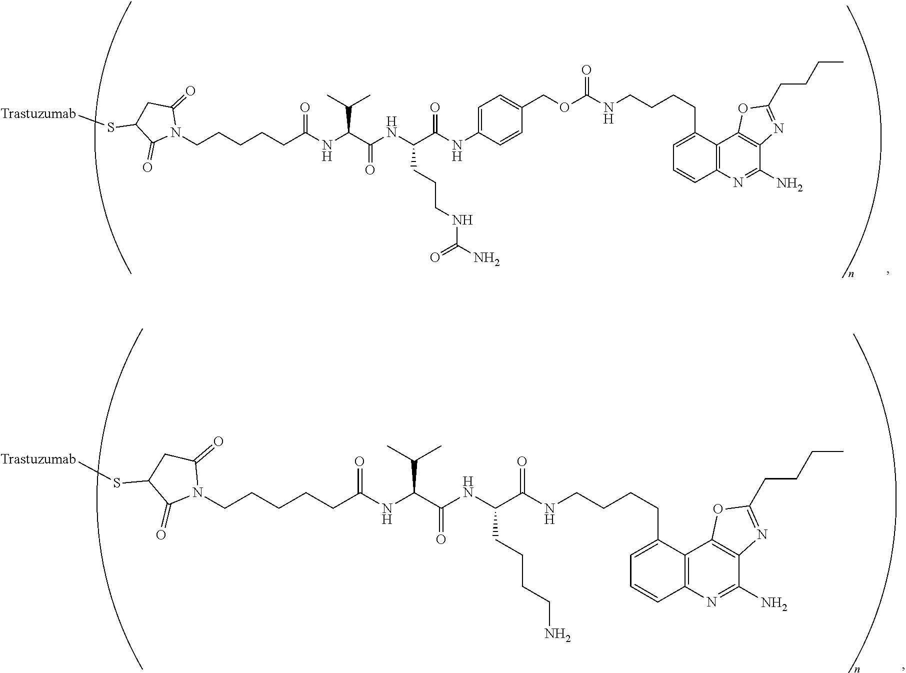

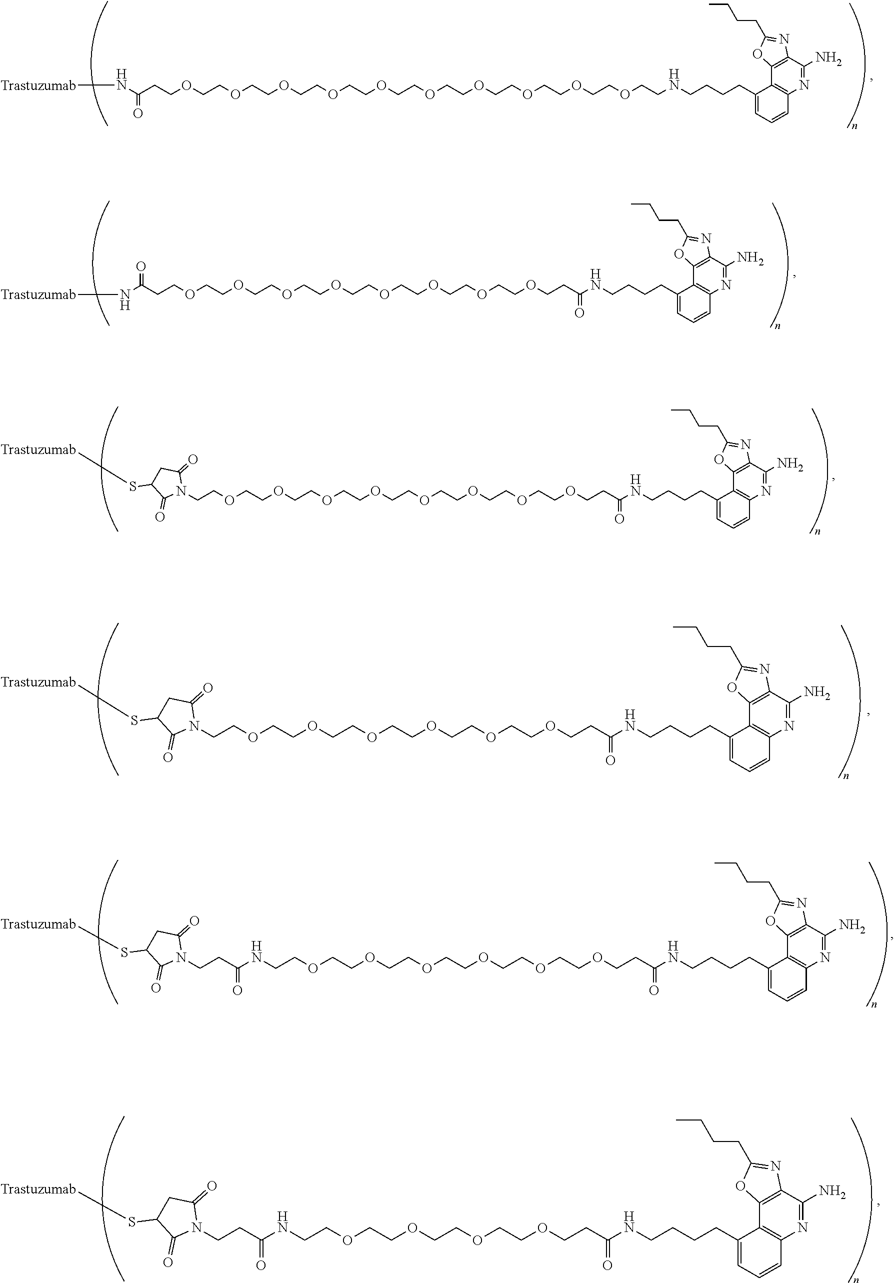

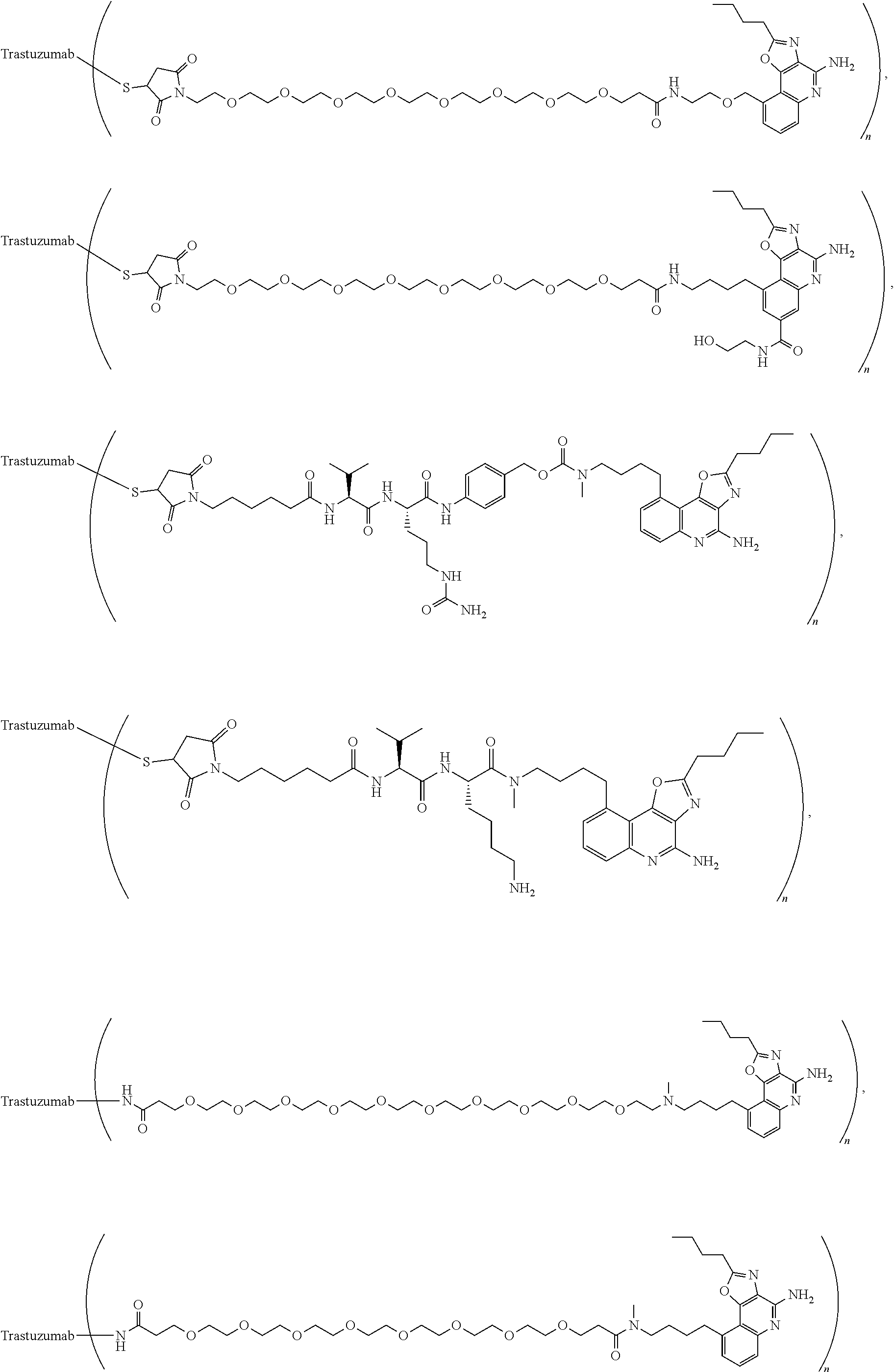

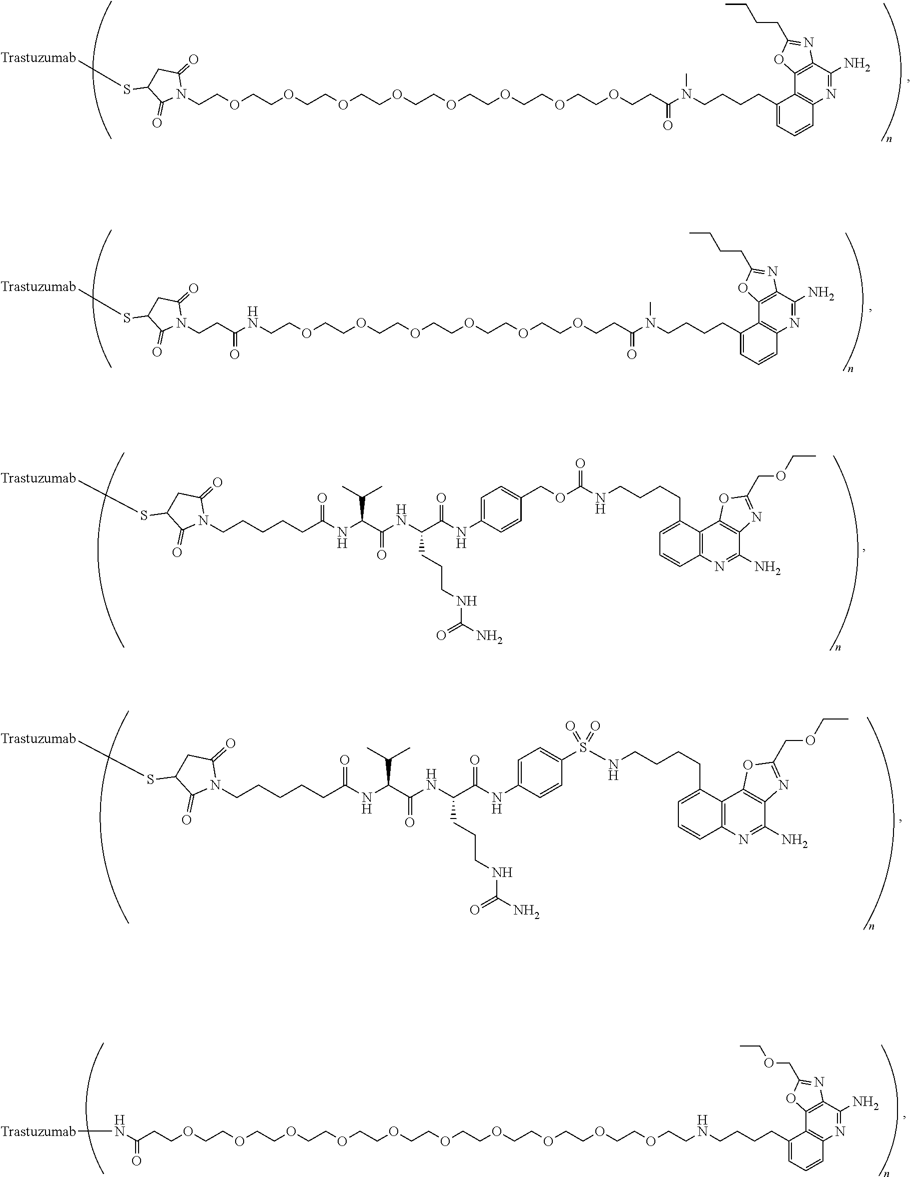

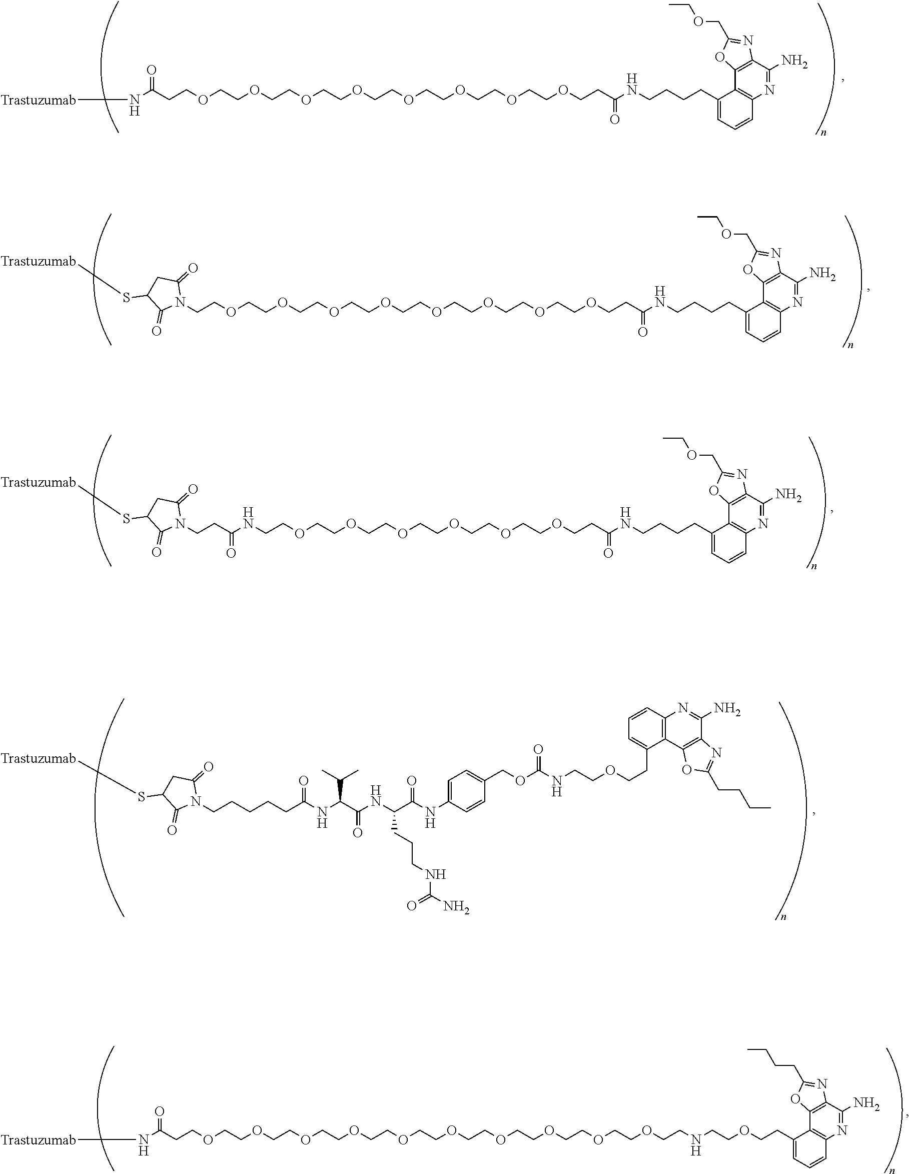

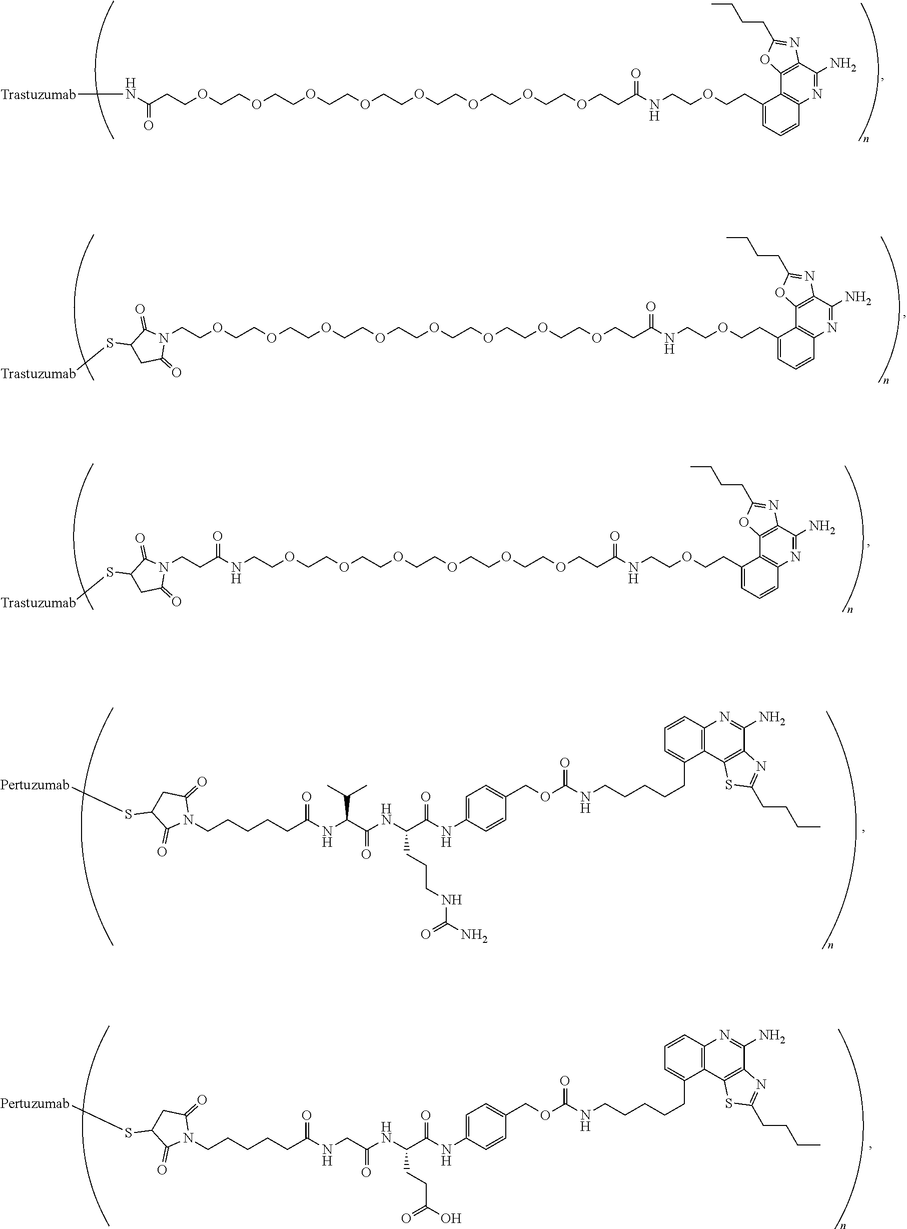

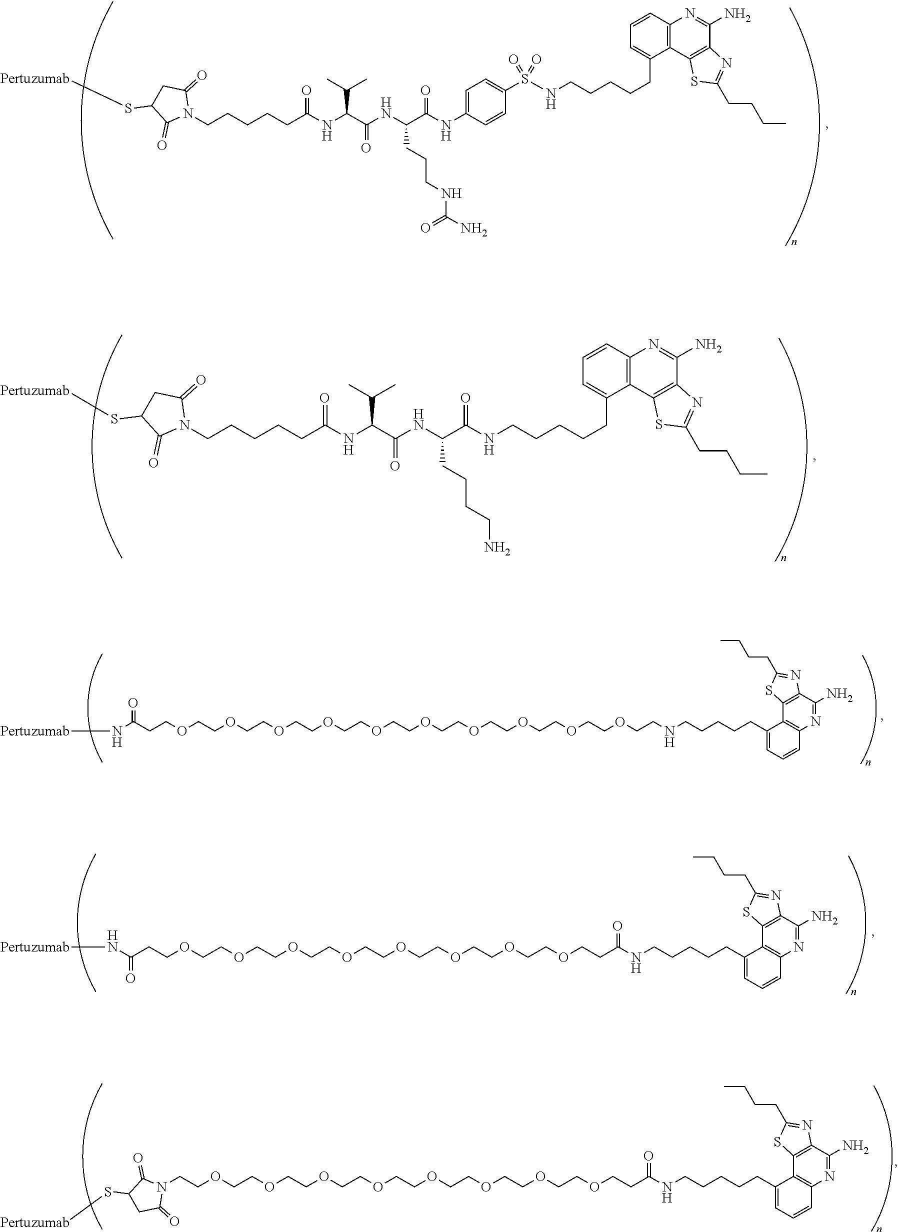

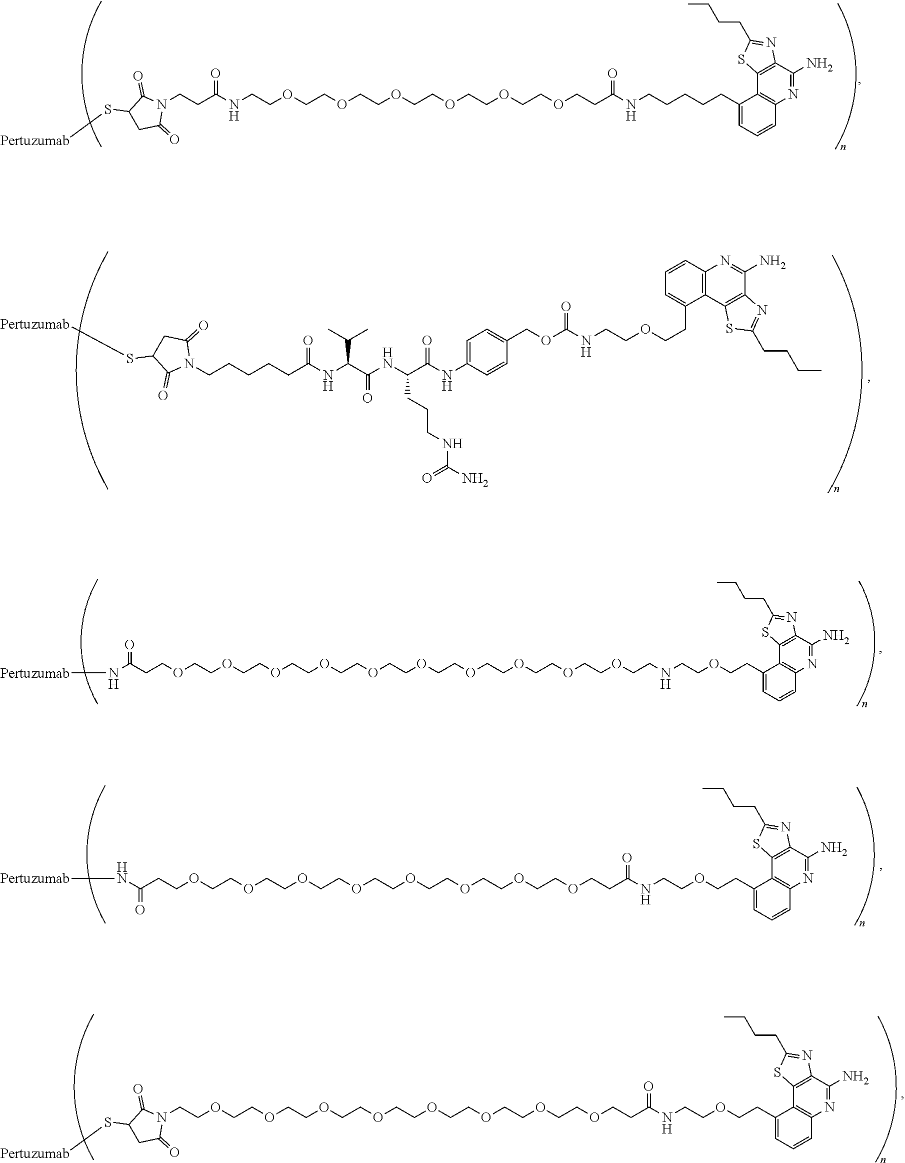

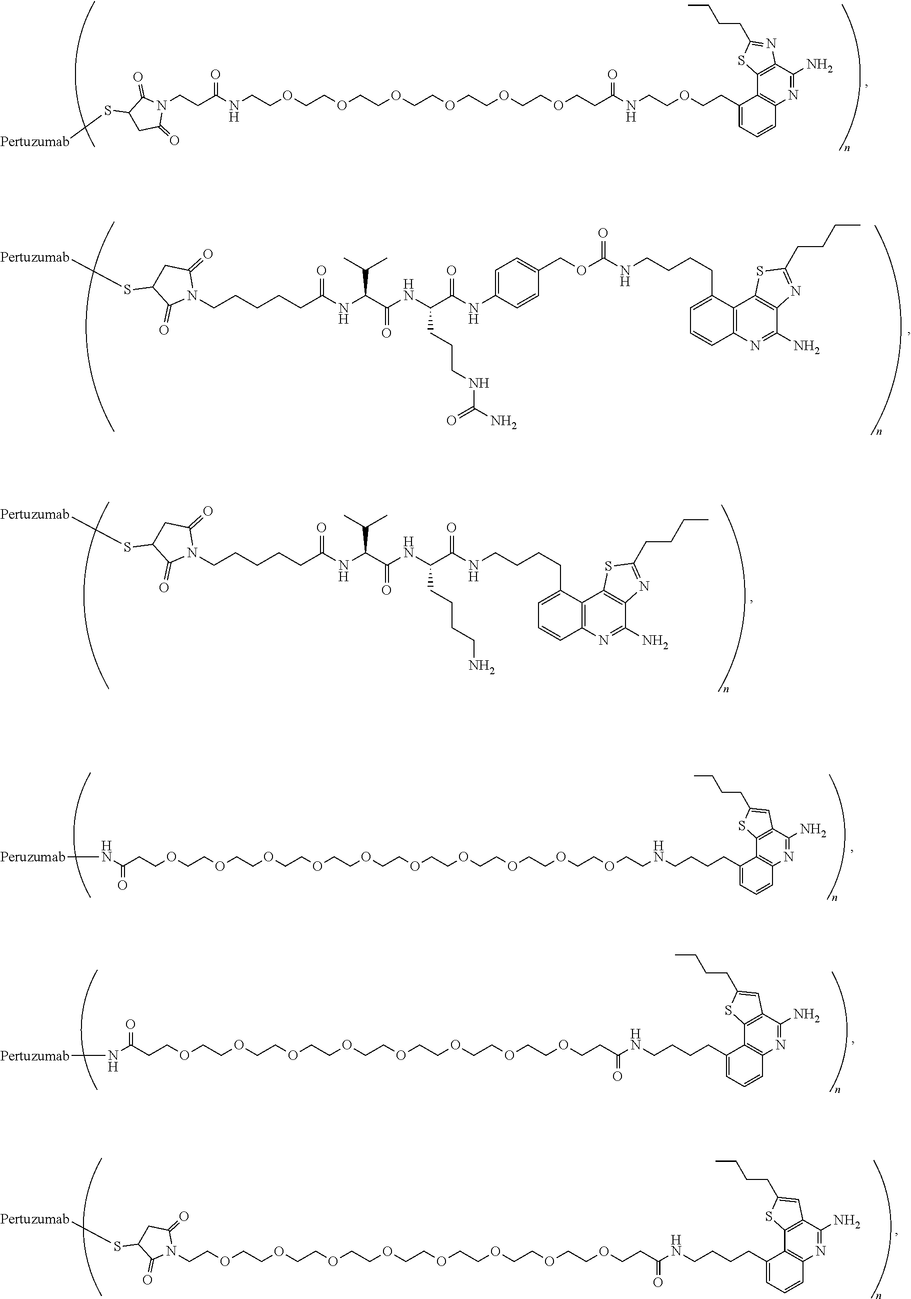

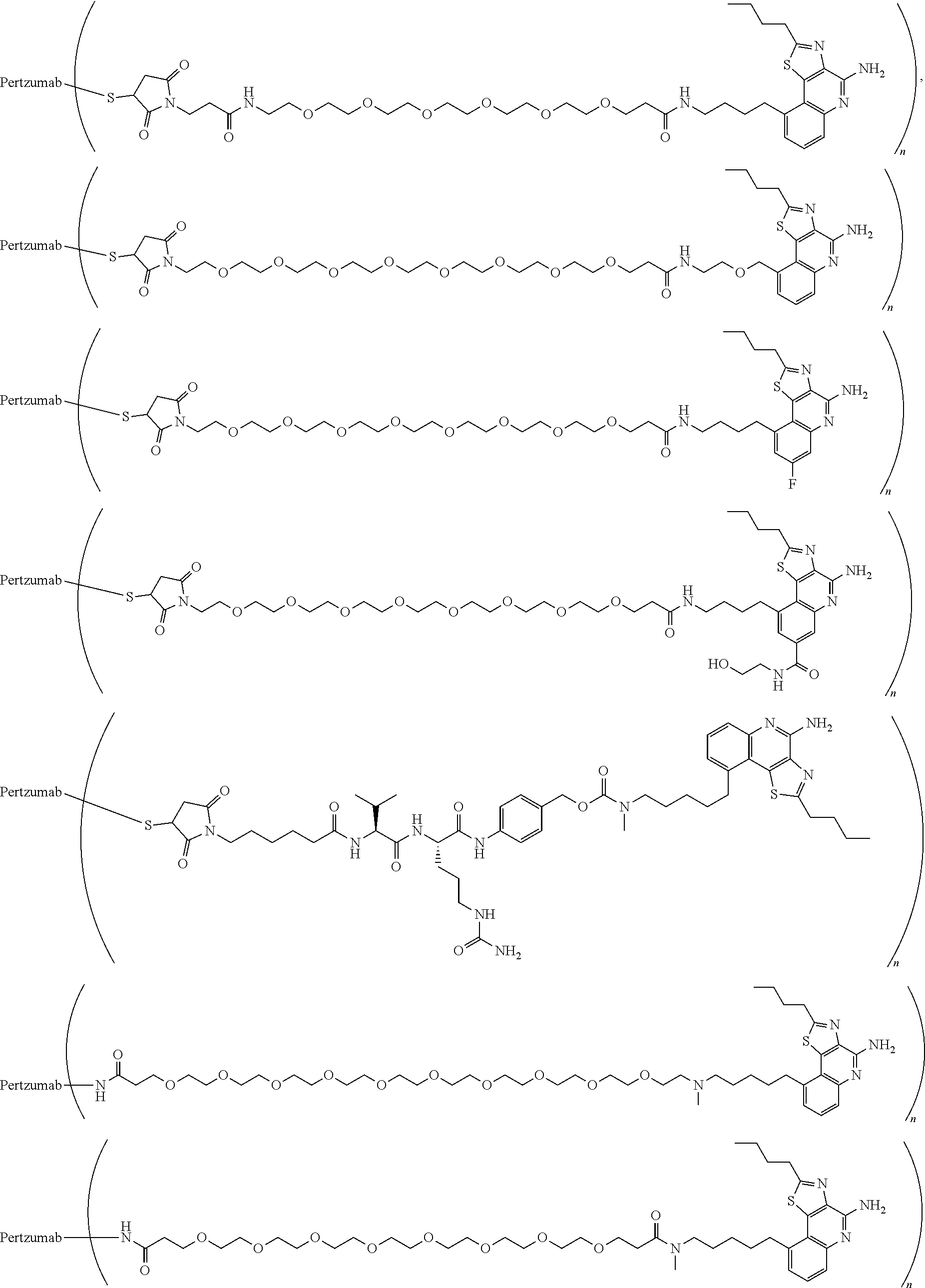

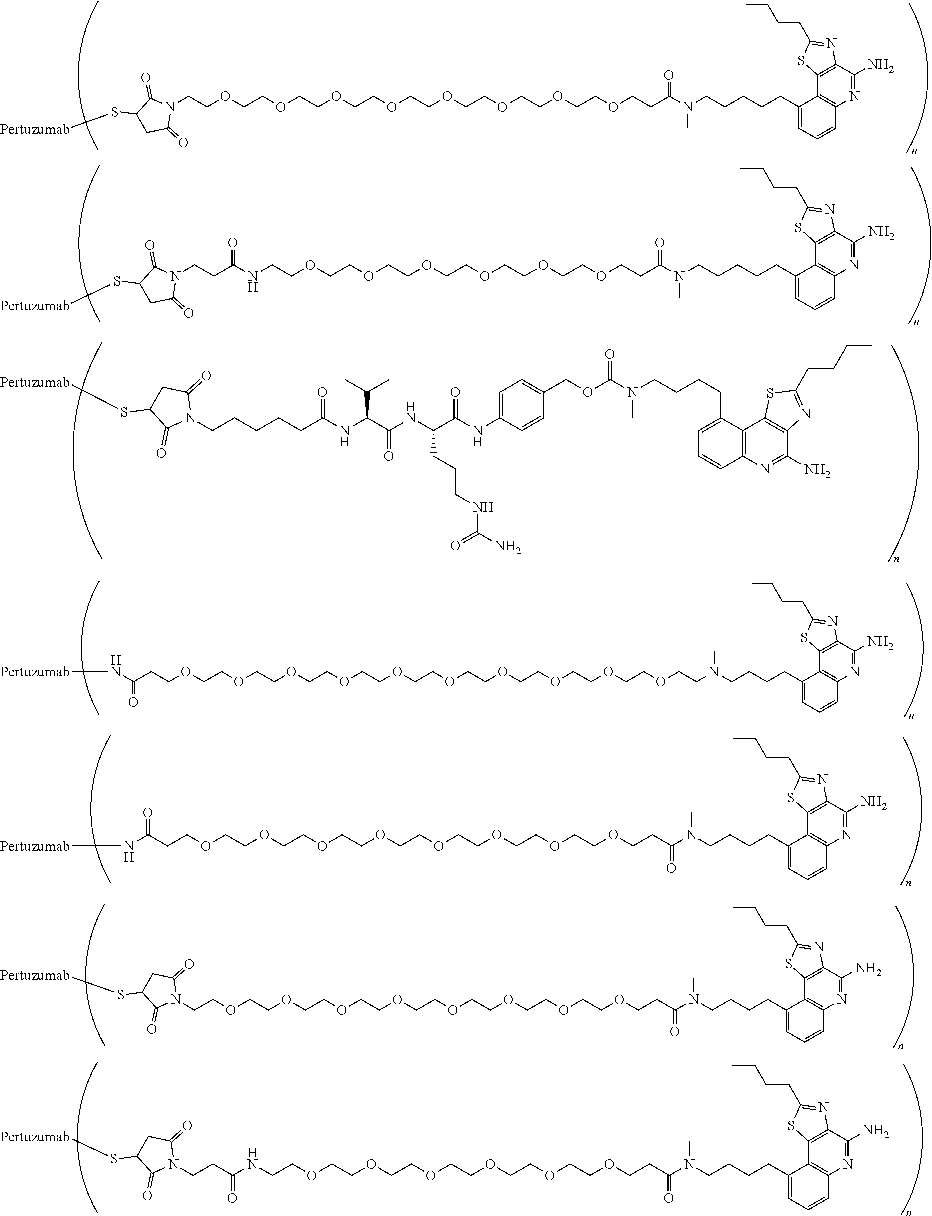

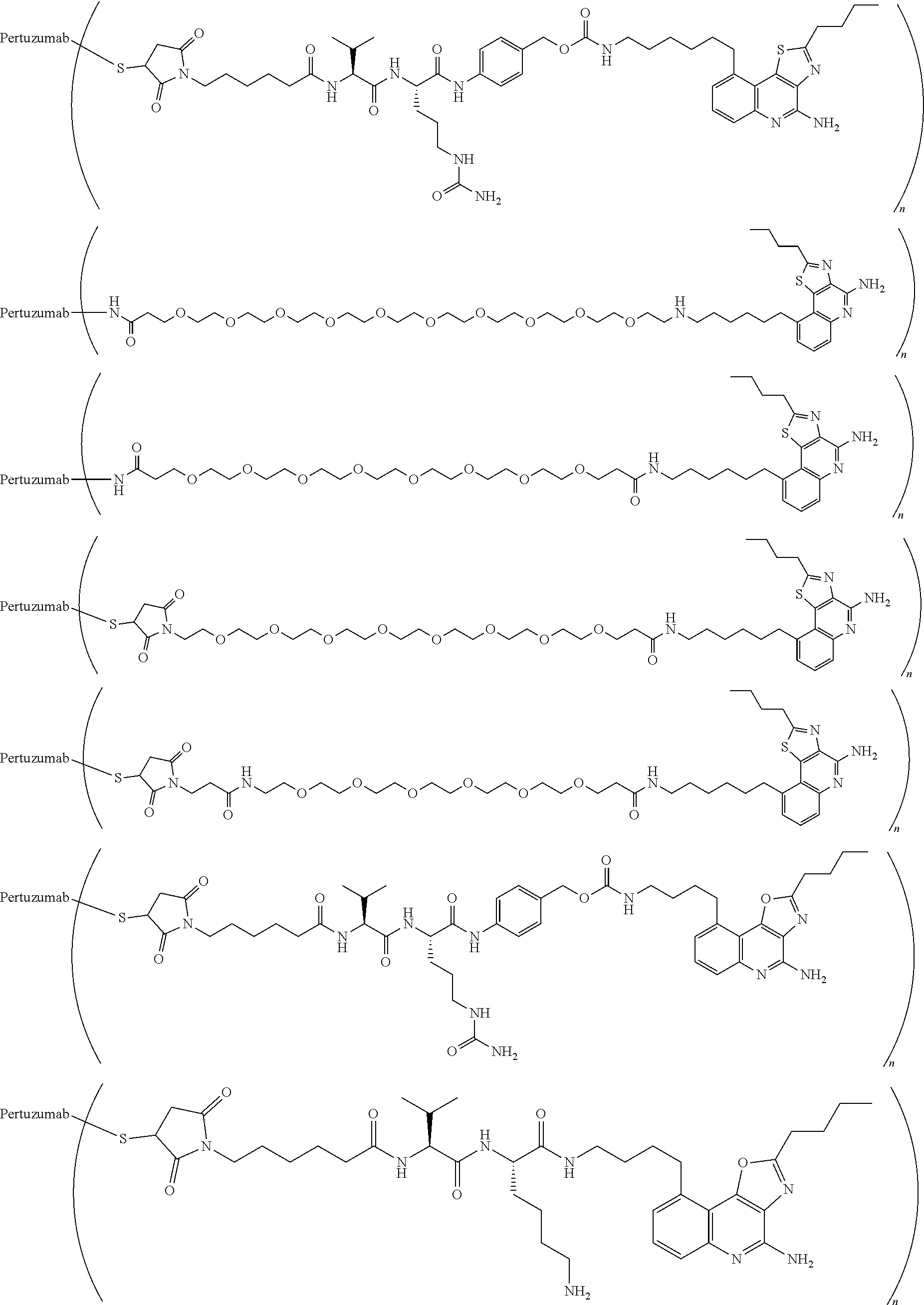

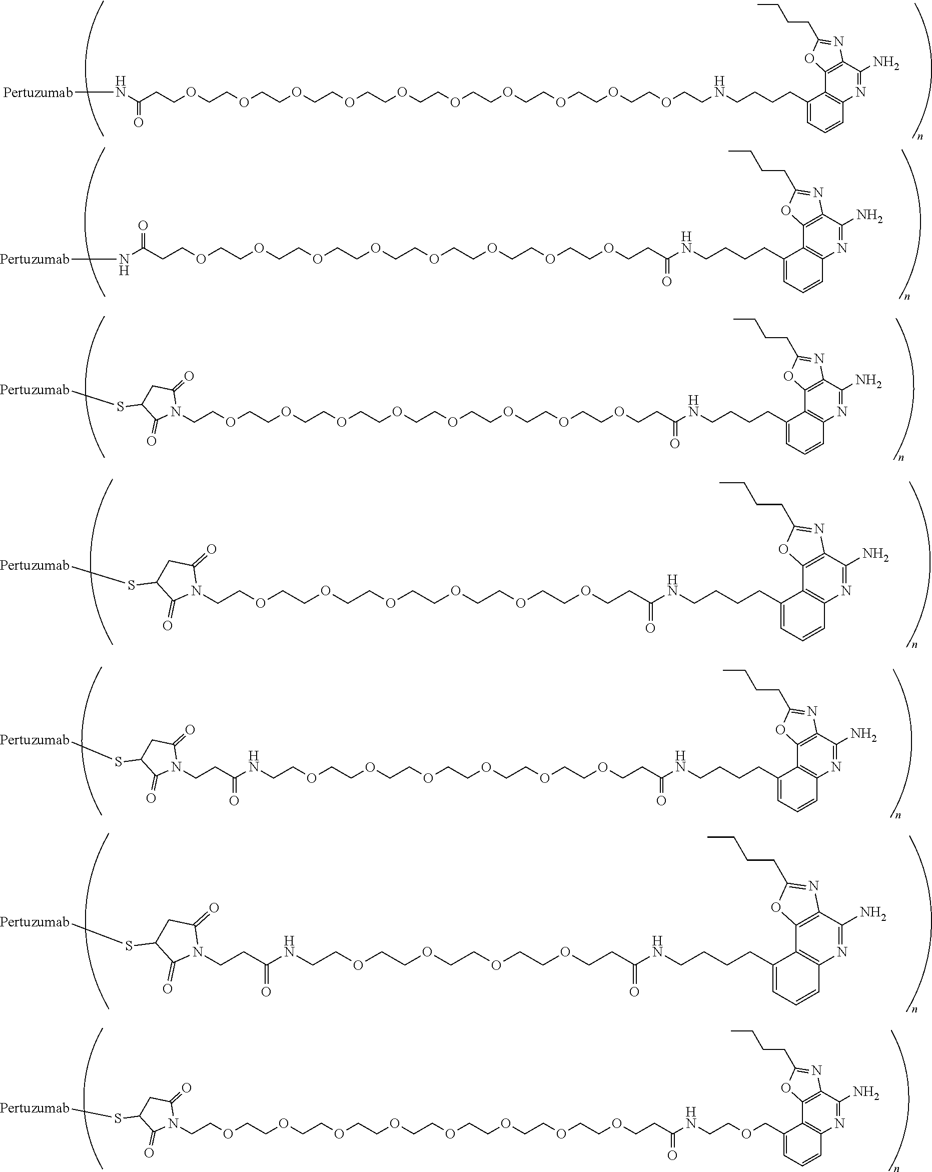

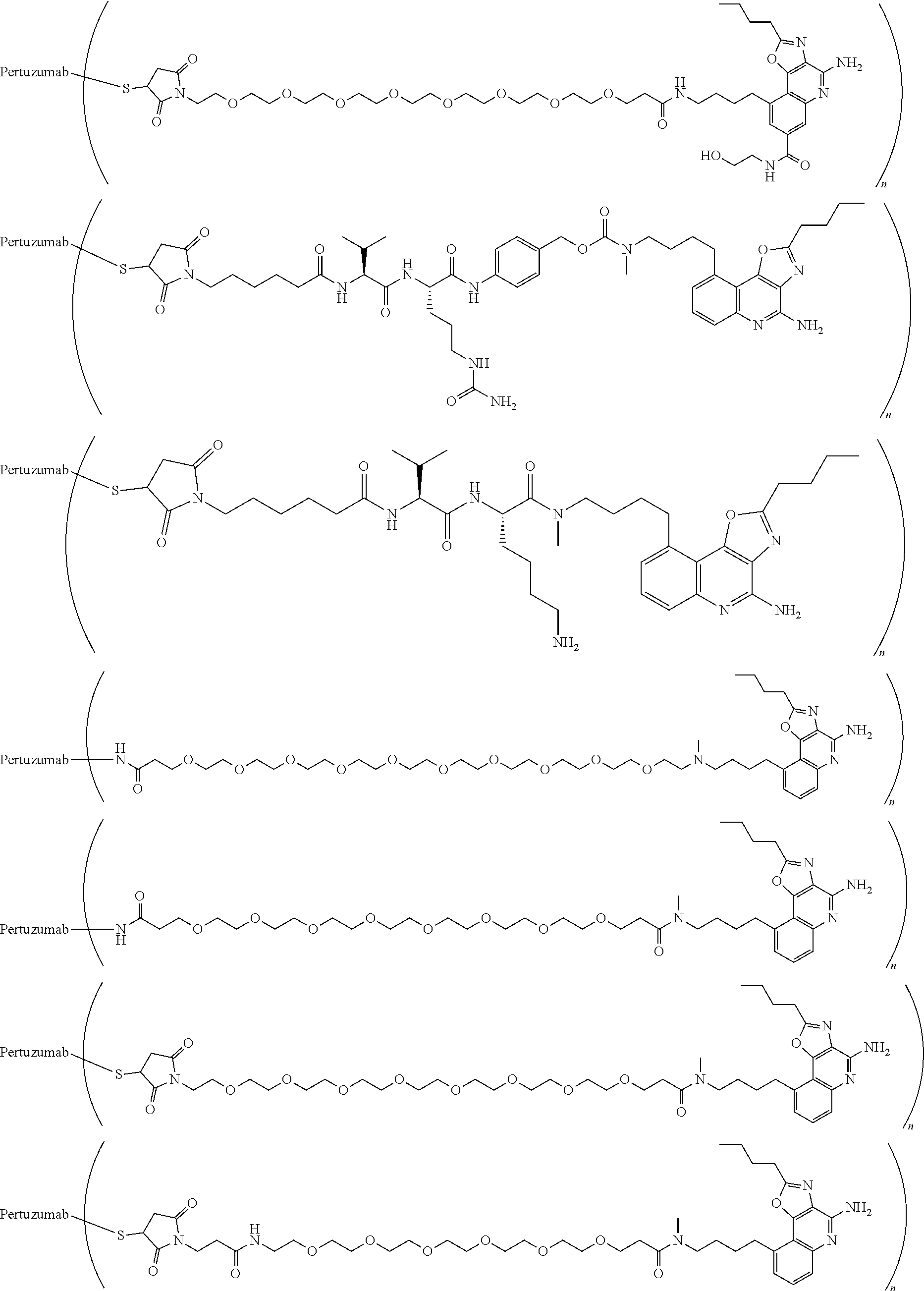

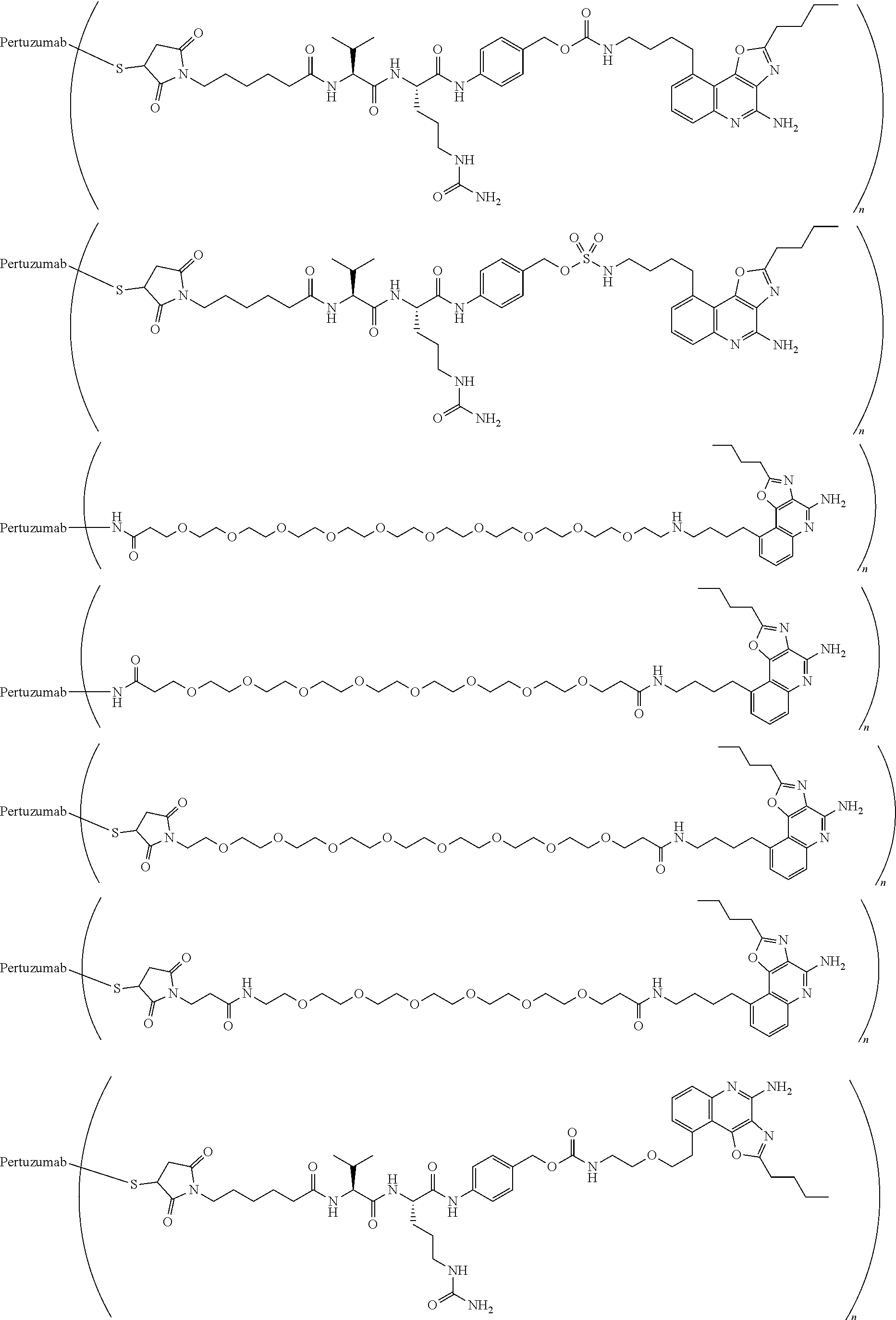

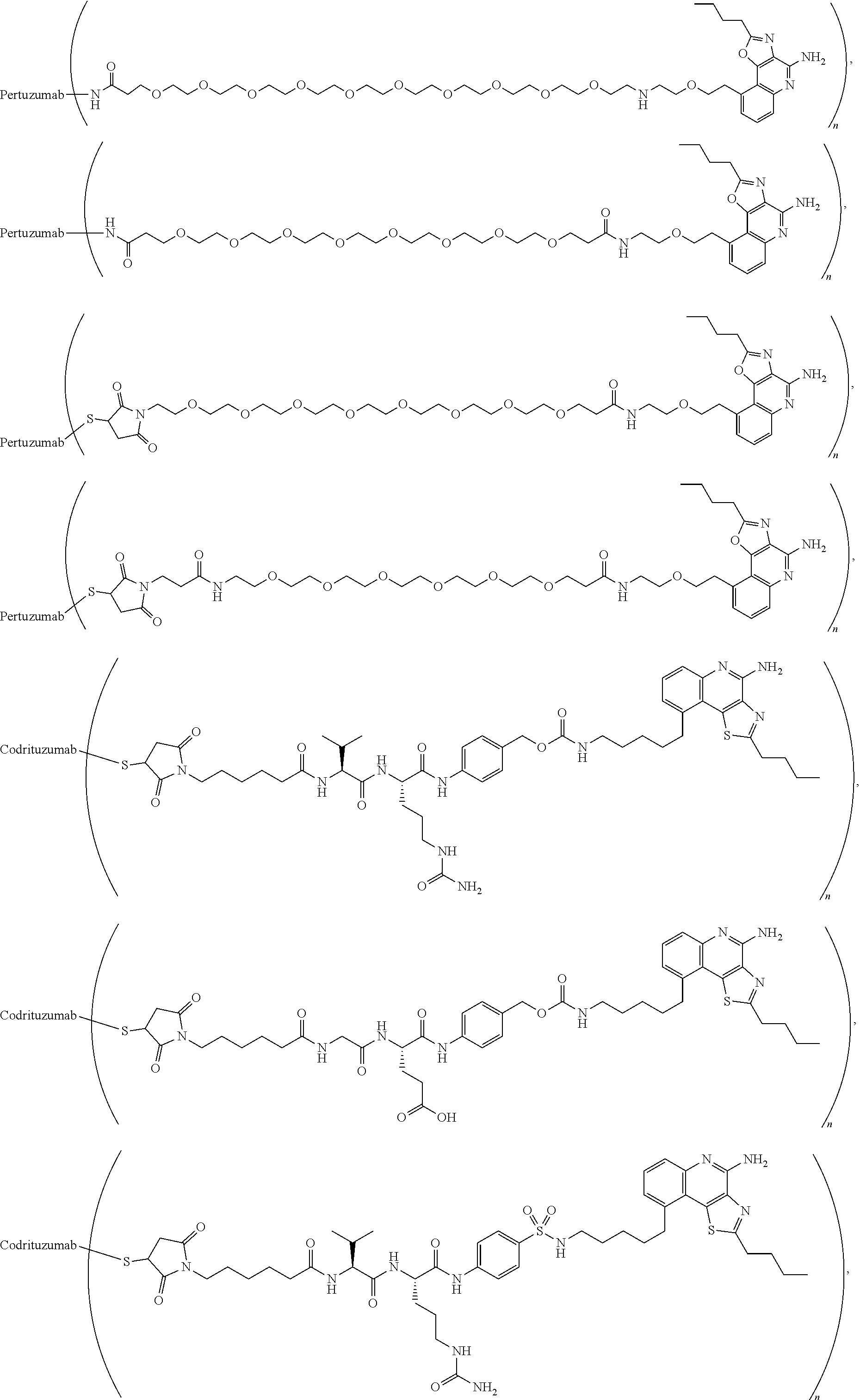

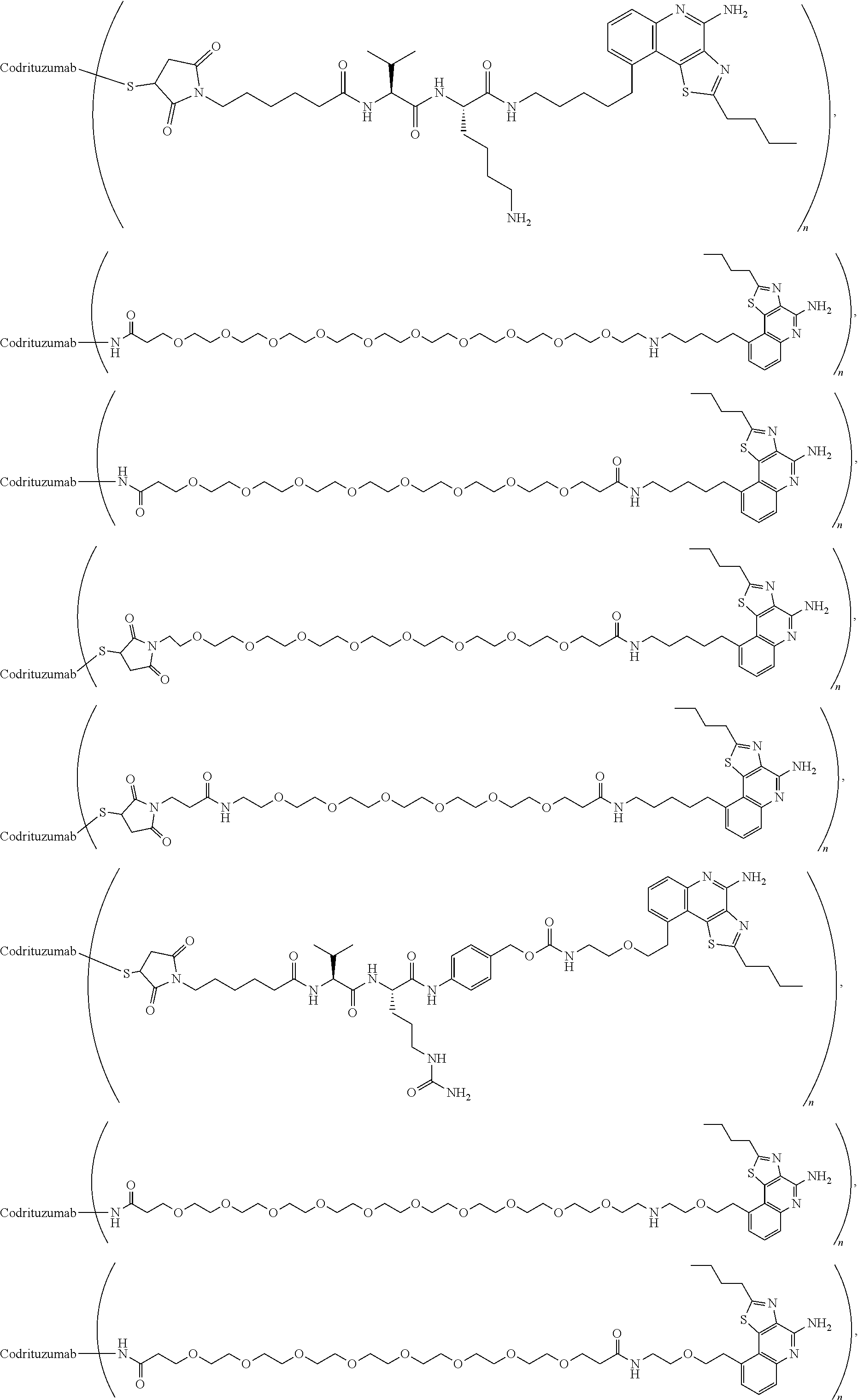

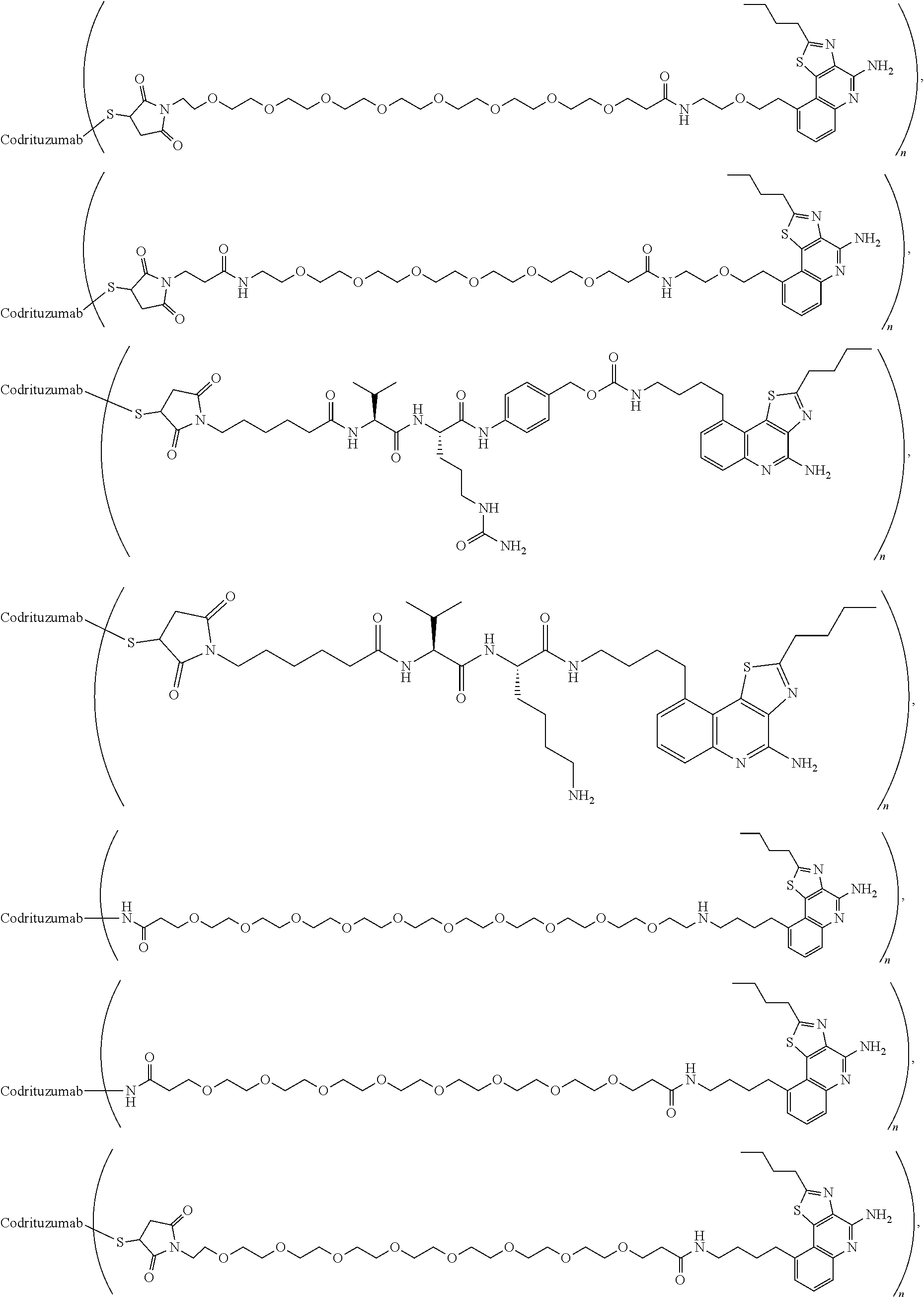

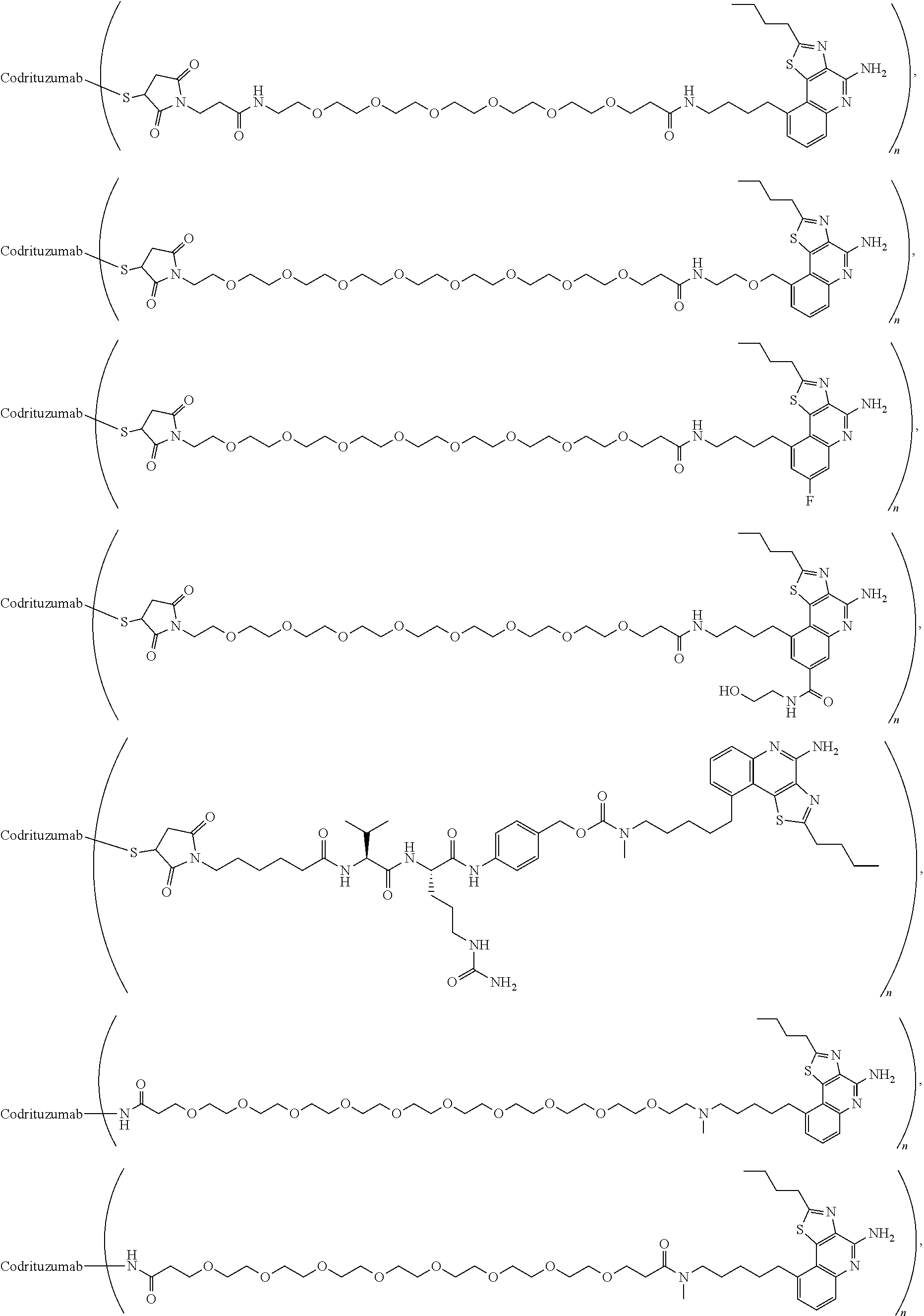

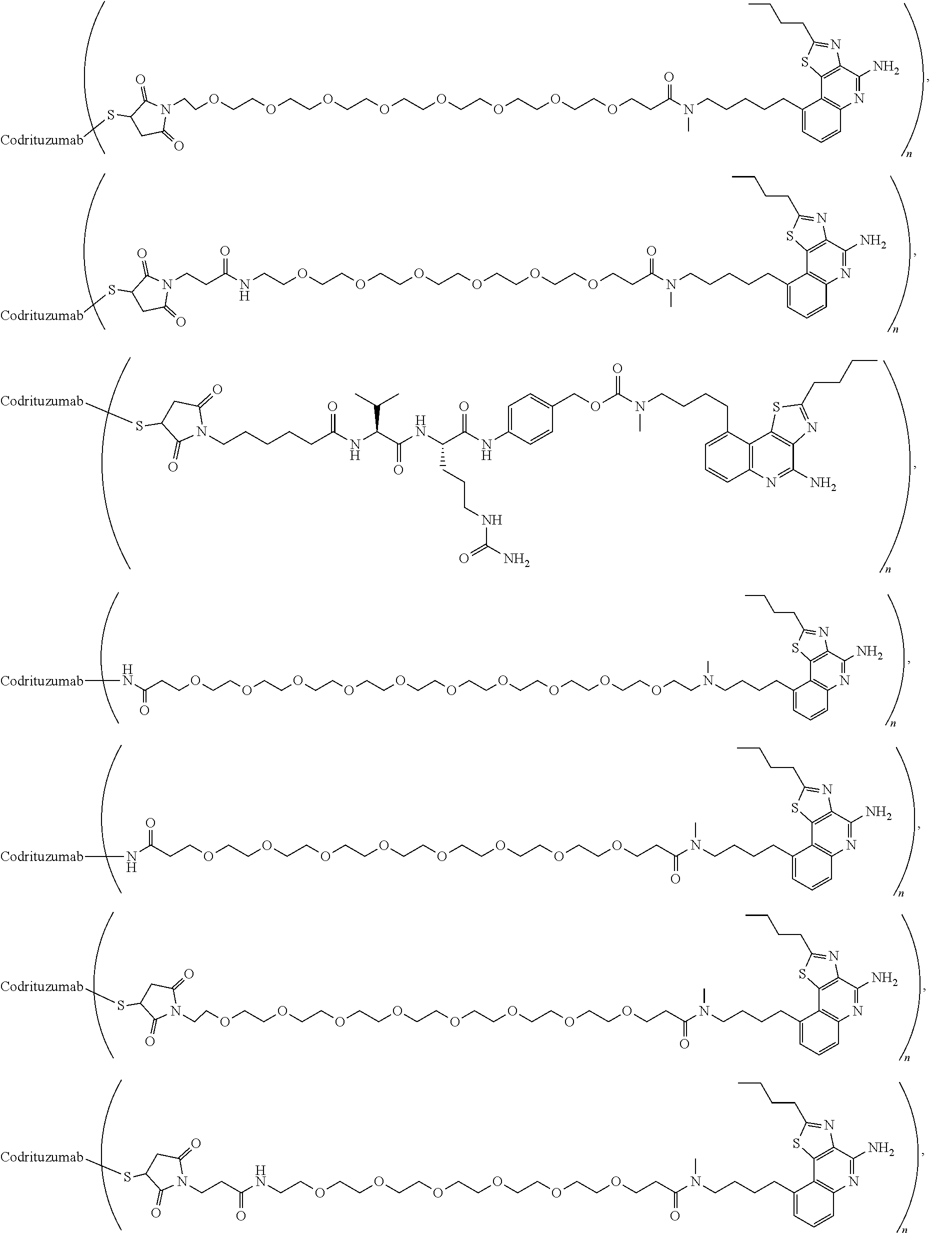

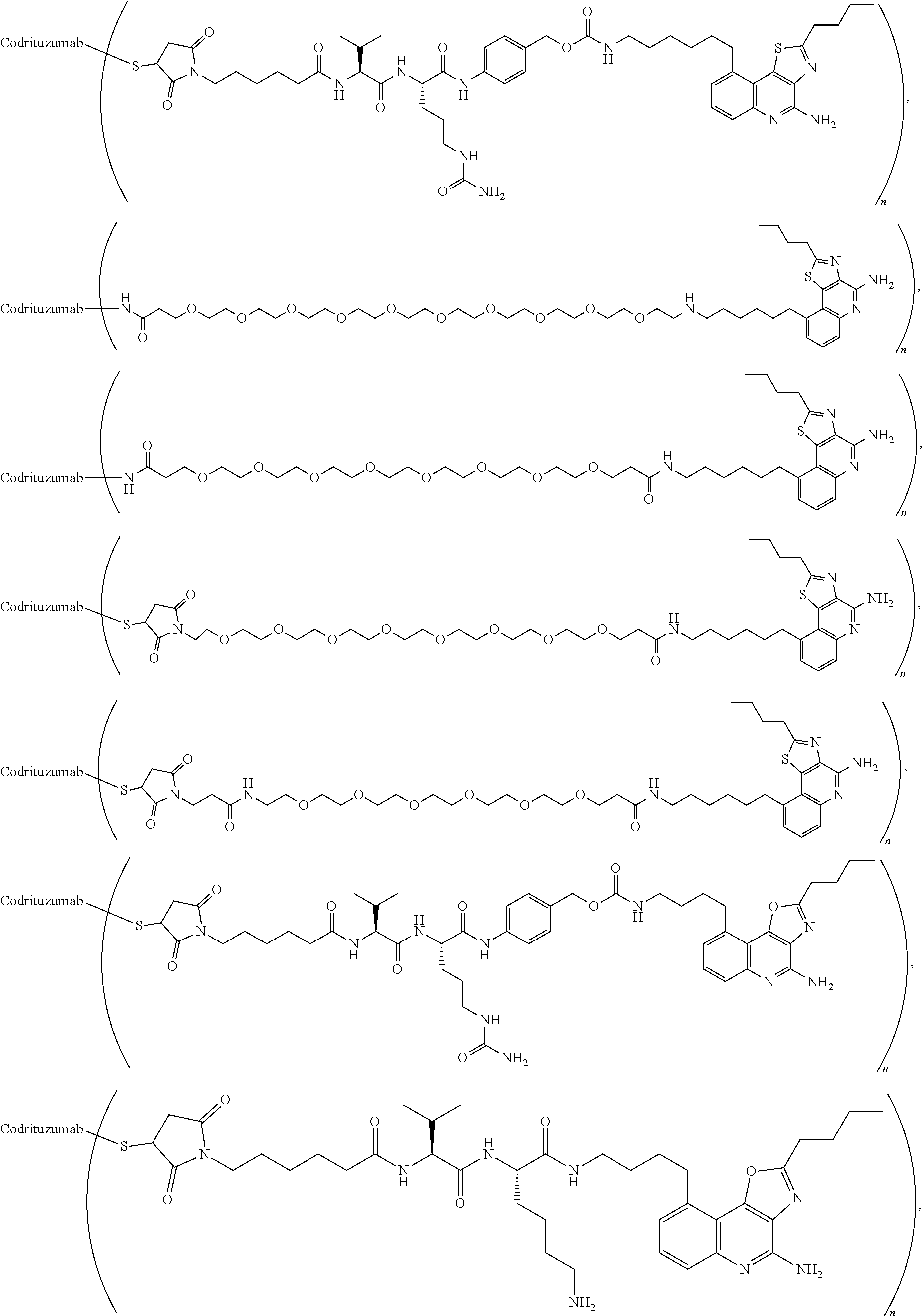

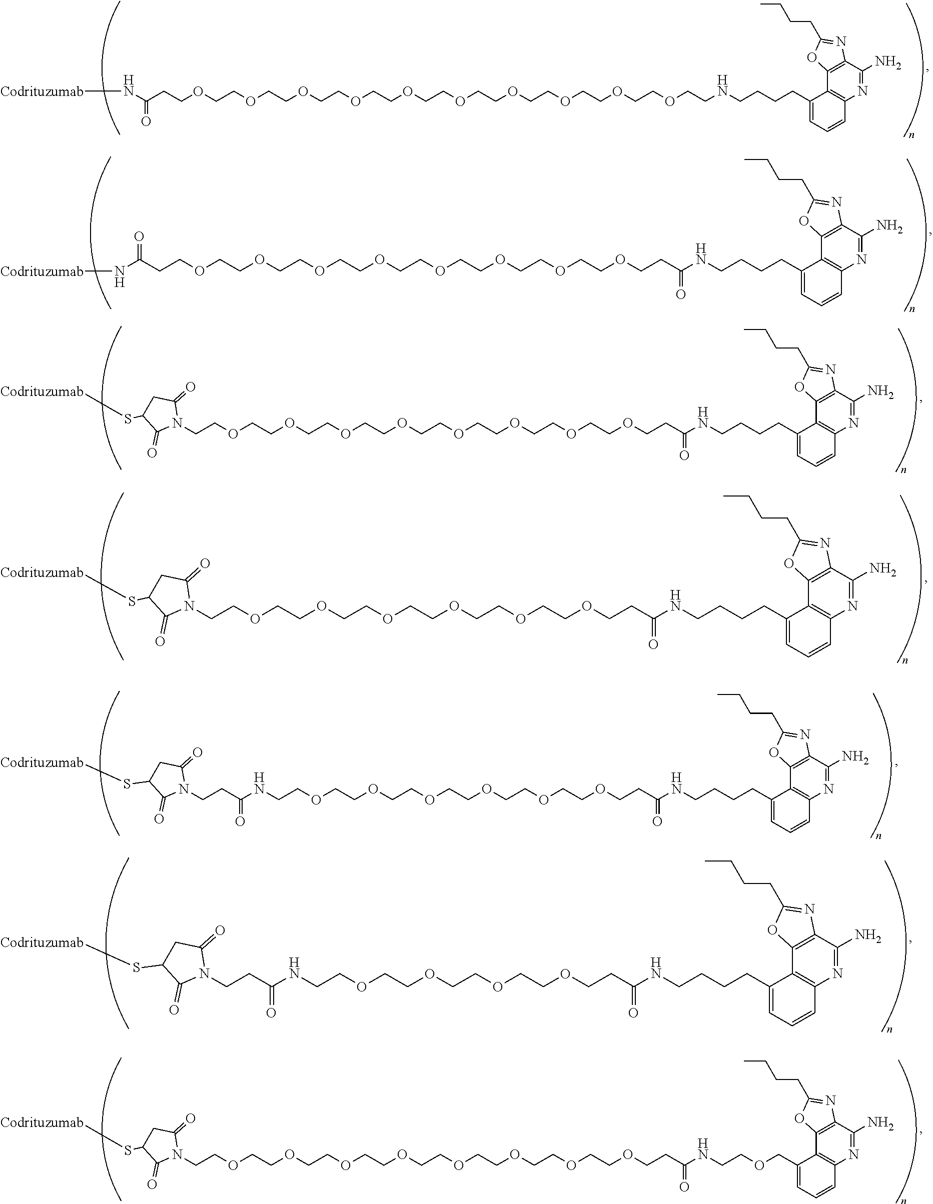

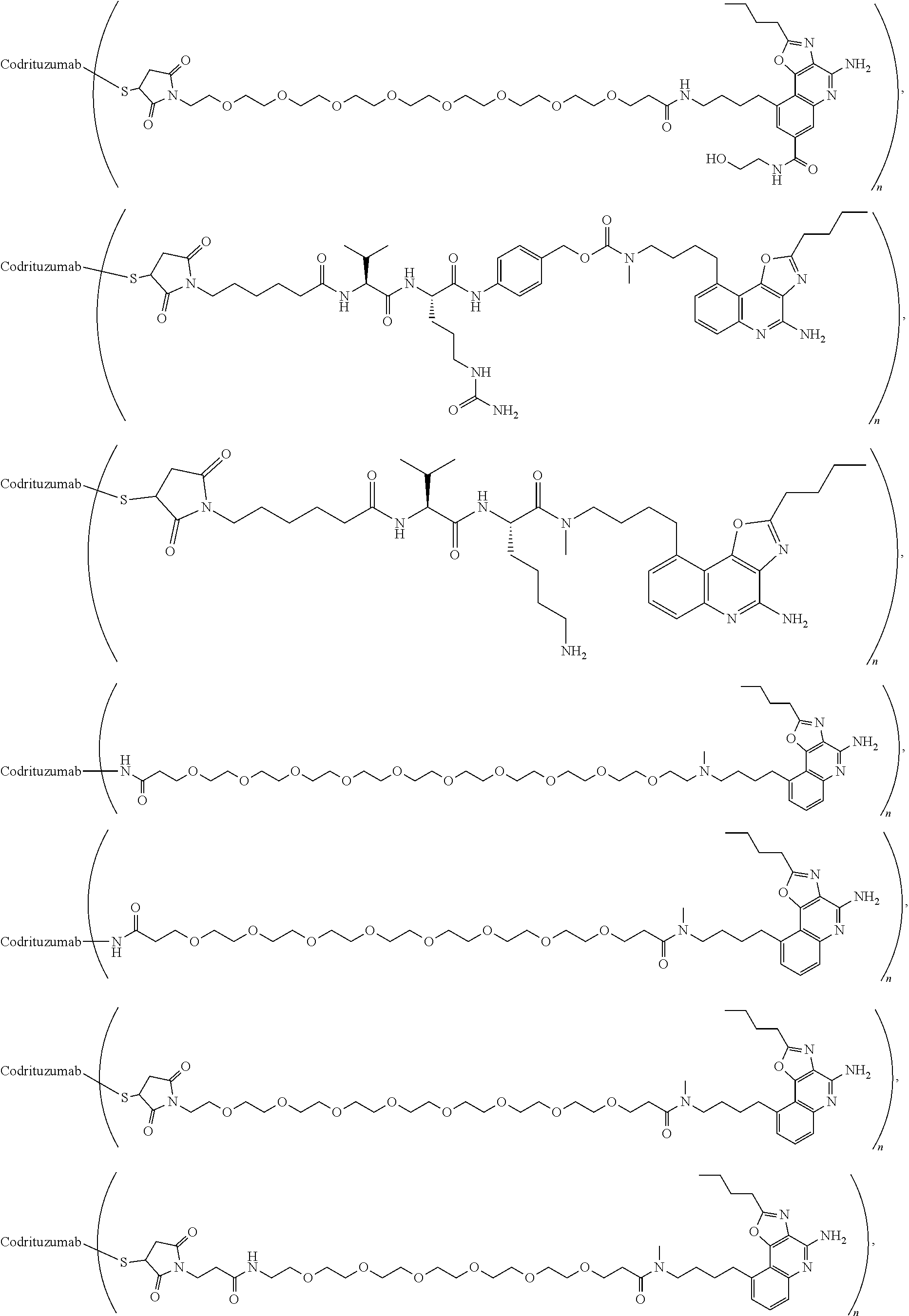

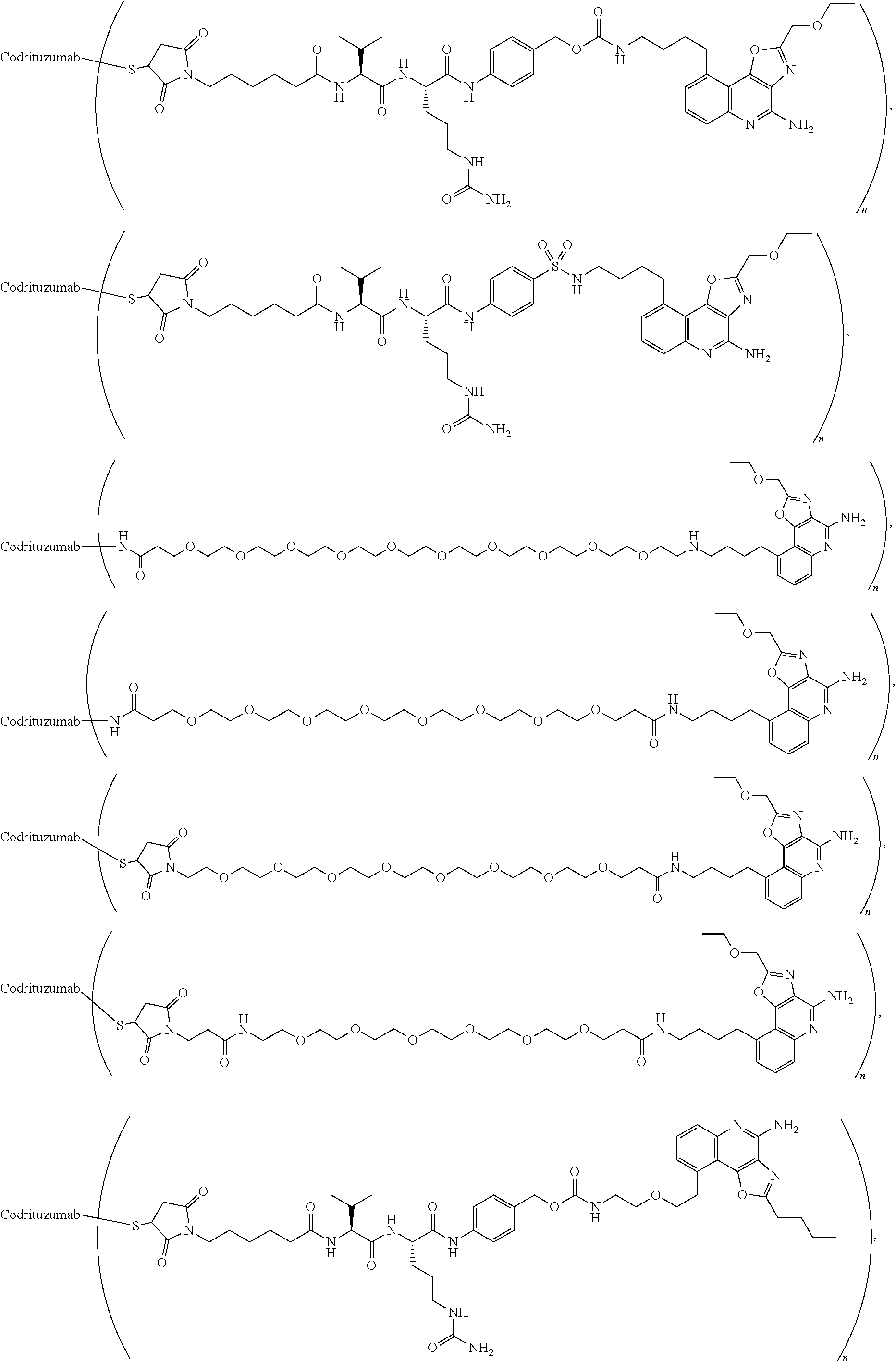

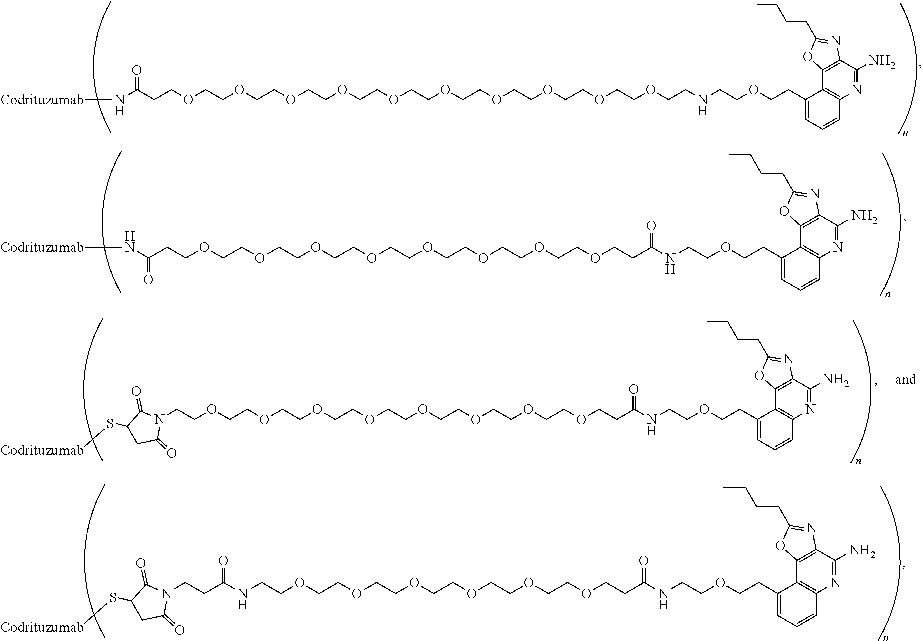

wherein
n is an integer or a decimal from 1 to 10.

In some embodiments, the compound or the conjugate thereof, or the isomer thereof, or the mixture thereof, or the pharmaceutically acceptable salt thereof described herein is selected from:

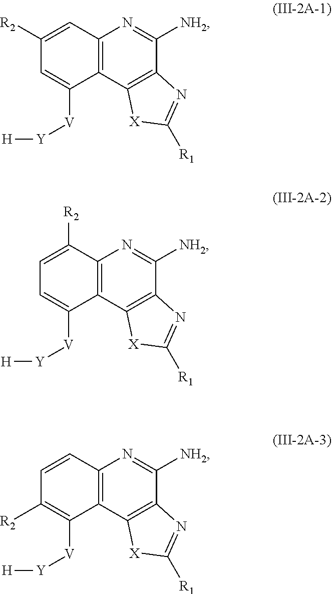

wherein
X is selected from —S— and —O—;
R₁ is selected from C₁-C₆ alkyl optionally substituted with 1, 2, or 3 R₁₋₁, each R₁₋₁ being independently selected from: hydrogen, chlorine, bromine, iodine, hydroxy, amino, C₁-C₃ alkyl, and C₃-C₆ cycloalkyl; the methylene units of R₁ are each independently unreplaced or replaced by a group selected from: —O—, —N(CH₃)—, and —NH—;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂₋₁, each R₂₋₁ being independently selected from: hydrogen, halogen, hydroxy, and amino;
V is selected from C₁-C₈alkylene, preferably C₁-C₆ alkylene, wherein one or more carbon atoms in the C₁-C₈ alkylene and the C₁-C₆ alkylene may be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
Y is selected from —N(R_{Y1})—;
R_{Y1} is selected from hydrogen and C₁-C₆ alkyl.

In certain preferred embodiments of the present disclosure, in the structures represented by formulas (III-2A-1), (III-2A-2) and (III-2A-3), R₁, R₂, V, and Y are as described in any one of the embodiments of (II-2A) of the present application.

In yet another aspect, the present disclosure provides a compound or a conjugate thereof, or an isomer thereof, or a mixture thereof, or a pharmaceutically acceptable salt thereof, wherein the compound has a structure represented by formula (III-3A):

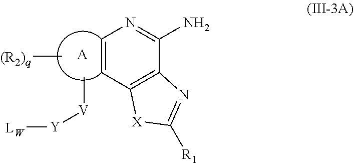

wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl; ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—,

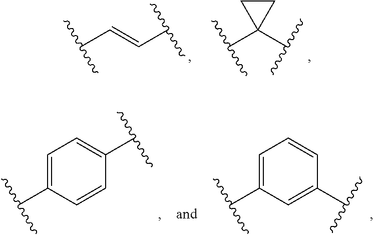

R_{V4} being selected from H, C₁-C₆ alkyl, and

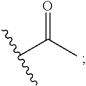 [SMILES: *C(C)=O]

Y is selected from —N(R_{Y1})—, —O—, —S—,

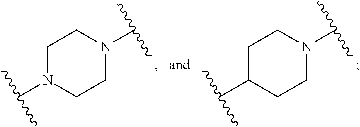 [SMILES: *N1CCN(*)CC1 ; *C1CCN(*)CC1]

R_{Y1} is selected from hydrogen and C₁-C₆ alkyl;
q is selected from 0, 1, 2, or 3;
p is selected from 1, 2, 3, 4, 5, 7, and 8;
L_{W} is -Tr-L₃-L₂-L₁;
Tr is selected from

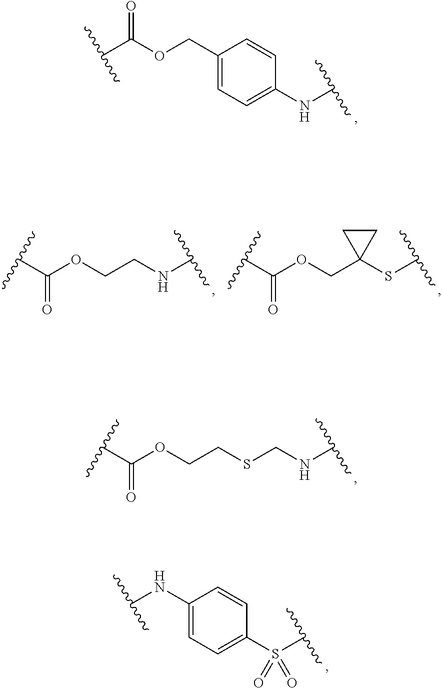 [SMILES: *Nc1ccc(COC(*)=O)cc1 ; *NCCOC(*)=O ; *SC1(COC(*)=O)CC1 ; *NCSCCOC(*)=O ; *Nc1ccc(S(*)(=O)=O)cc1]

and a single bond;
L₁ is selected from

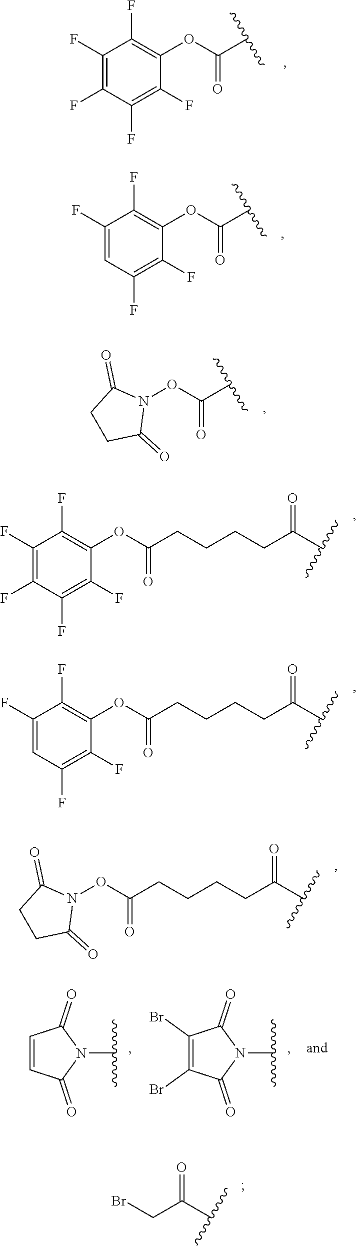

L₂ is selected from

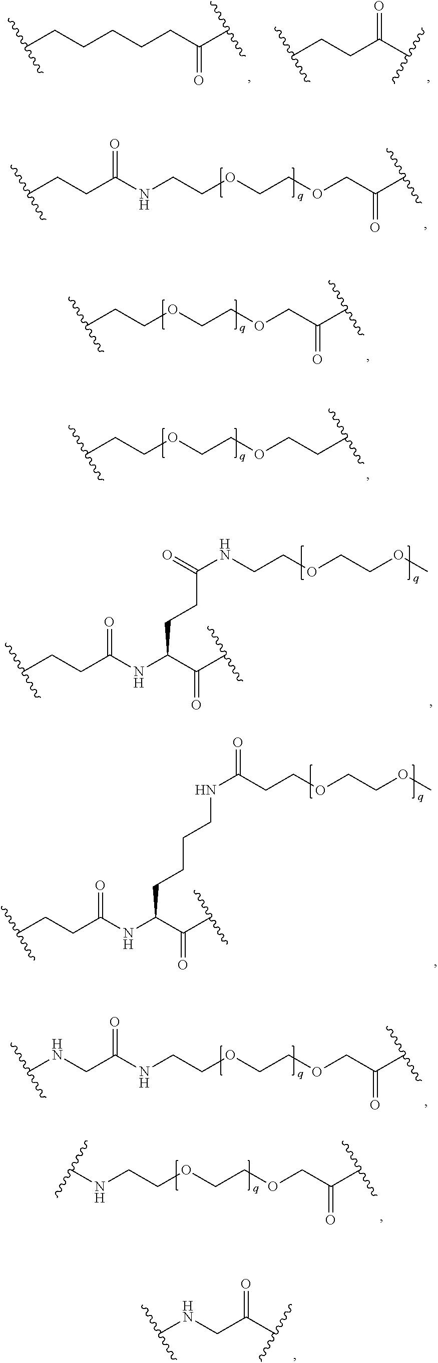

and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, glycine-glutamine, glycine-aspartic acid-, glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

In certain preferred embodiments of the present disclosure, in the structure represented by formula (III-3A), R₁, X₂, ring A, V, Y, Lw, and q are as described in any one of the embodiments of (III-1A), (III-2A), (III-2B) or (III-2C) of the present application.

In some embodiments, in the compound or the conjugate thereof, or the isomer thereof, or the mixture thereof, or the pharmaceutically acceptable salt thereof of the present disclosure, the compound is represented by formulas (III-3A-1) and (III-3A-2):

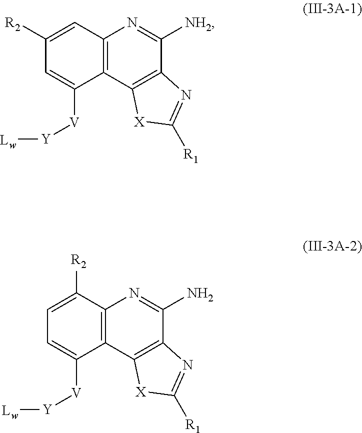

wherein
X is selected from —S— and —O—;
R₁ is selected from C₁-C₆ alkyl optionally substituted with 1, 2, or 3 RI-1, each R₁₋₁ being independently selected from: hydrogen, chlorine, bromine, iodine, hydroxy, amino, C₁-C₃ alkyl, and C₃-C₆ cycloalkyl; the methylene units of R₁ are each independently unreplaced or replaced by a group selected from: —O—, —N(CH₃)—, and —NH—;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂_1; each R₂-1 is independently selected from: hydrogen, halogen, hydroxy, and amino;
V is selected from C₁-C₈alkylene, preferably C₁-C₆ alkylene, wherein one or more carbon atoms in the C₁-C₈ alkylene and the C₁-C₆ alkylene may be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
Y is selected from —N(R_{Y1})—;
R_{Y1} is selected from hydrogen and C₁-C₆ alkyl;
L_{W} is selected from

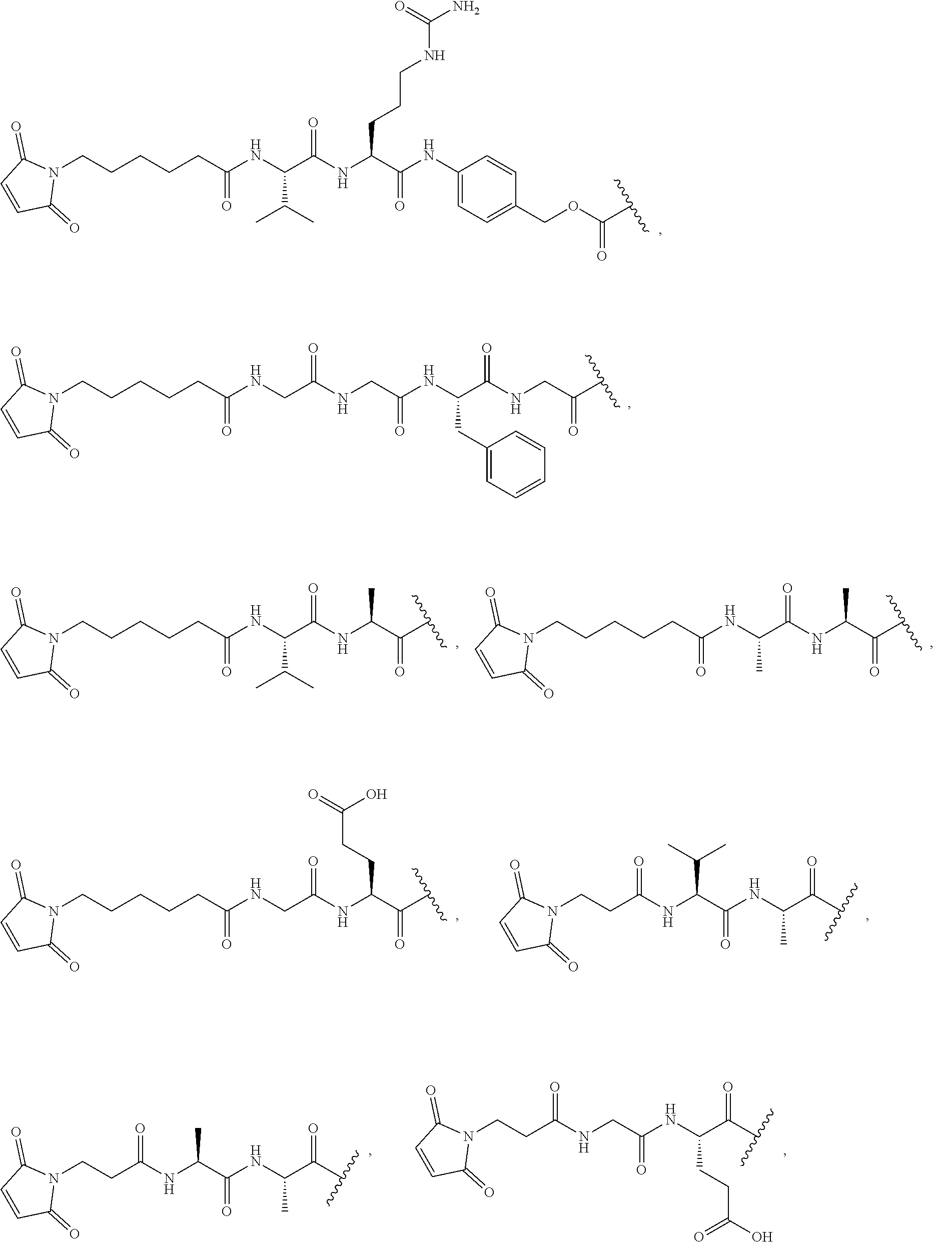

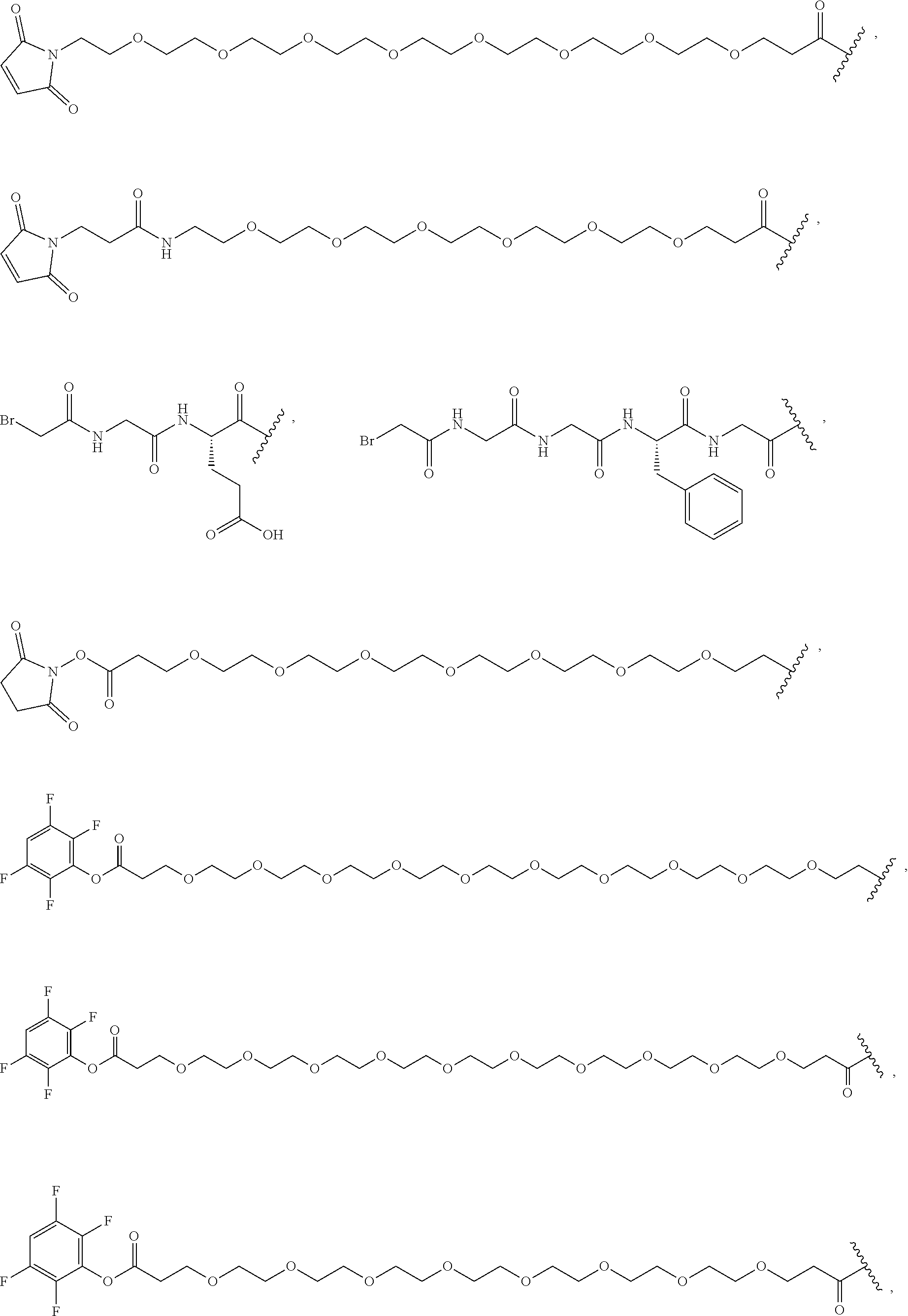

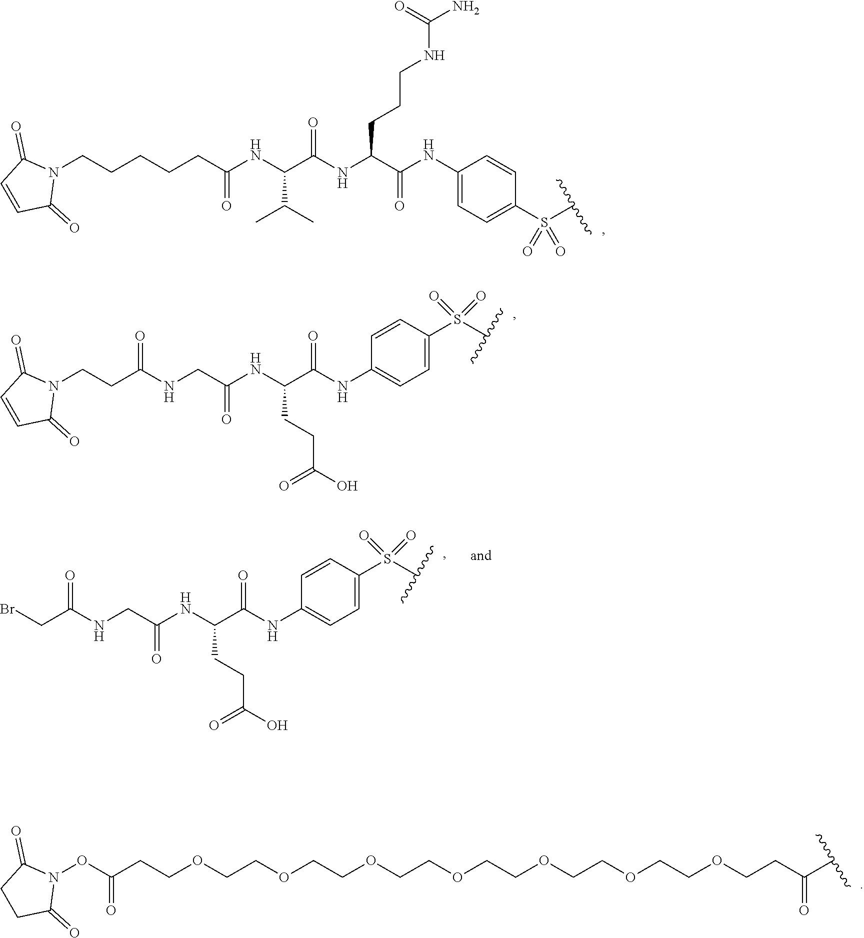 [SMILES: *S(=O)(=O)c1ccc(NC(=O)[C@H](CCCNC(N)=O)NC(=O)[C@@H](NC(=O)CCCCCN2C(=O)C=CC2=O)C(C)C)cc1 ; *S(=O)(=O)c1ccc(NC(=O)[C@H](CCC(=O)O)NC(=O)CNC(=O)CCN2C(=O)C=CC2=O)cc1 ; *S(=O)(=O)c1ccc(NC(=O)[C@H](CCC(=O)O)NC(=O)CNC(=O)CBr)cc1 ; *C(=O)CCOCCOCCOCCOCCOCCOCCOCCC(=O)ON1C(=O)CCC1=O]

In certain preferred embodiments of the present disclosure, in the structures represented by formulas (III-3A-1) and (III-3A-2), R₁, R₂, V, Y, and Lw are as described in any one of the embodiments of (III-1A), (III-2A), (III-2B) or (III-2C) of the present application.

In yet another aspect, the present disclosure provides a compound or a conjugate thereof, or an isomer thereof, or a mixture thereof, or a pharmaceutically acceptable salt thereof, selected from:

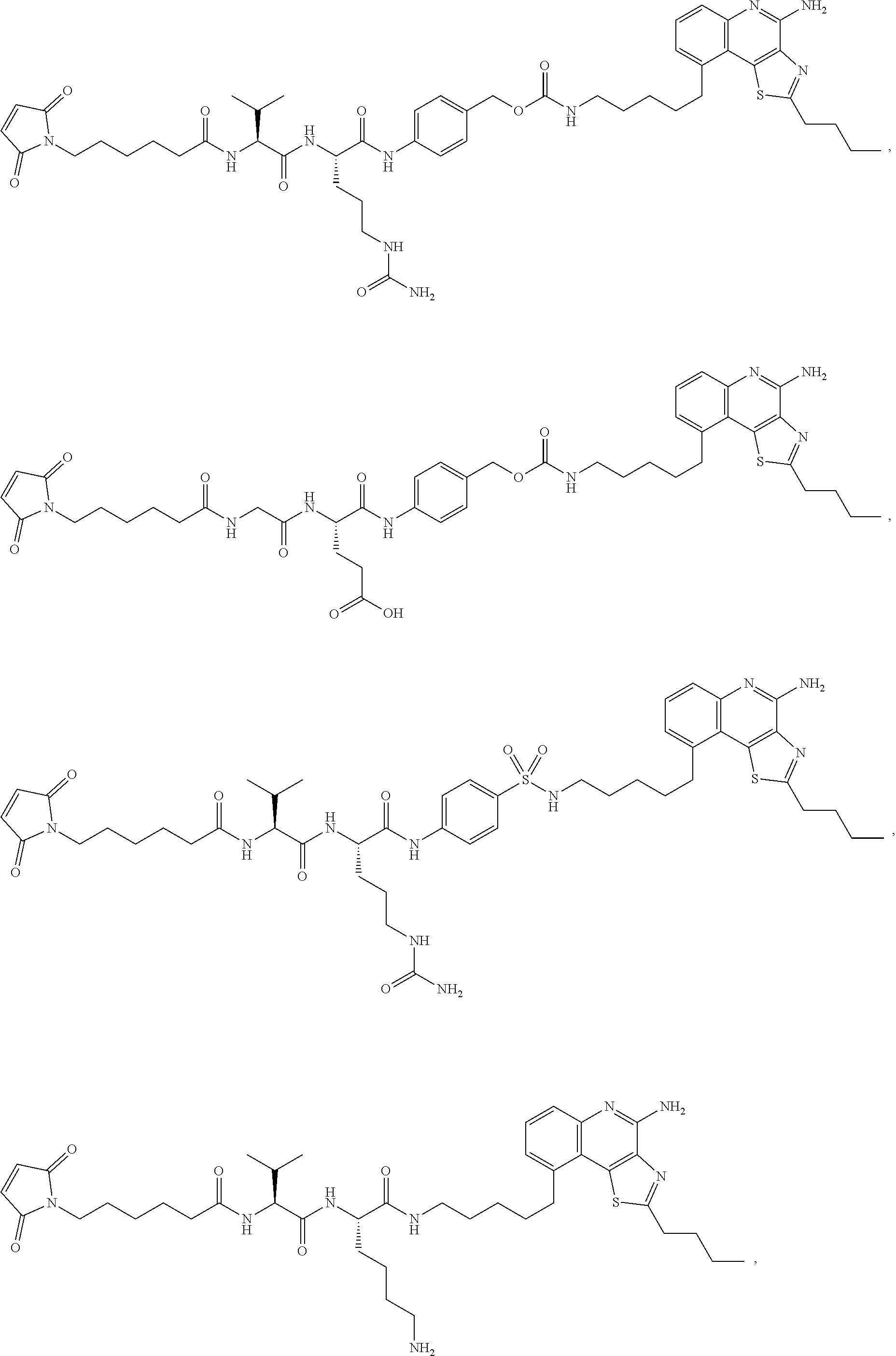 [SMILES: CCCCc1nc2c(N)nc3cccc(CCCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCNC(=O)OCc4ccc(NC(=O)[C@H](CCC(=O)O)NC(=O)CNC(=O)CCCCCN5C(=O)C=CC5=O)cc4)c3c2s1 ; CCCCc1nc2c(N)nc3cccc(CCCCCNS(=O)(=O)c4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCNC(=O)[C@H](CCCCN)NC(=O)[C@@H](NC(=O)CCCCCN4C(=O)C=CC4=O)C(C)C)c3c2s1]

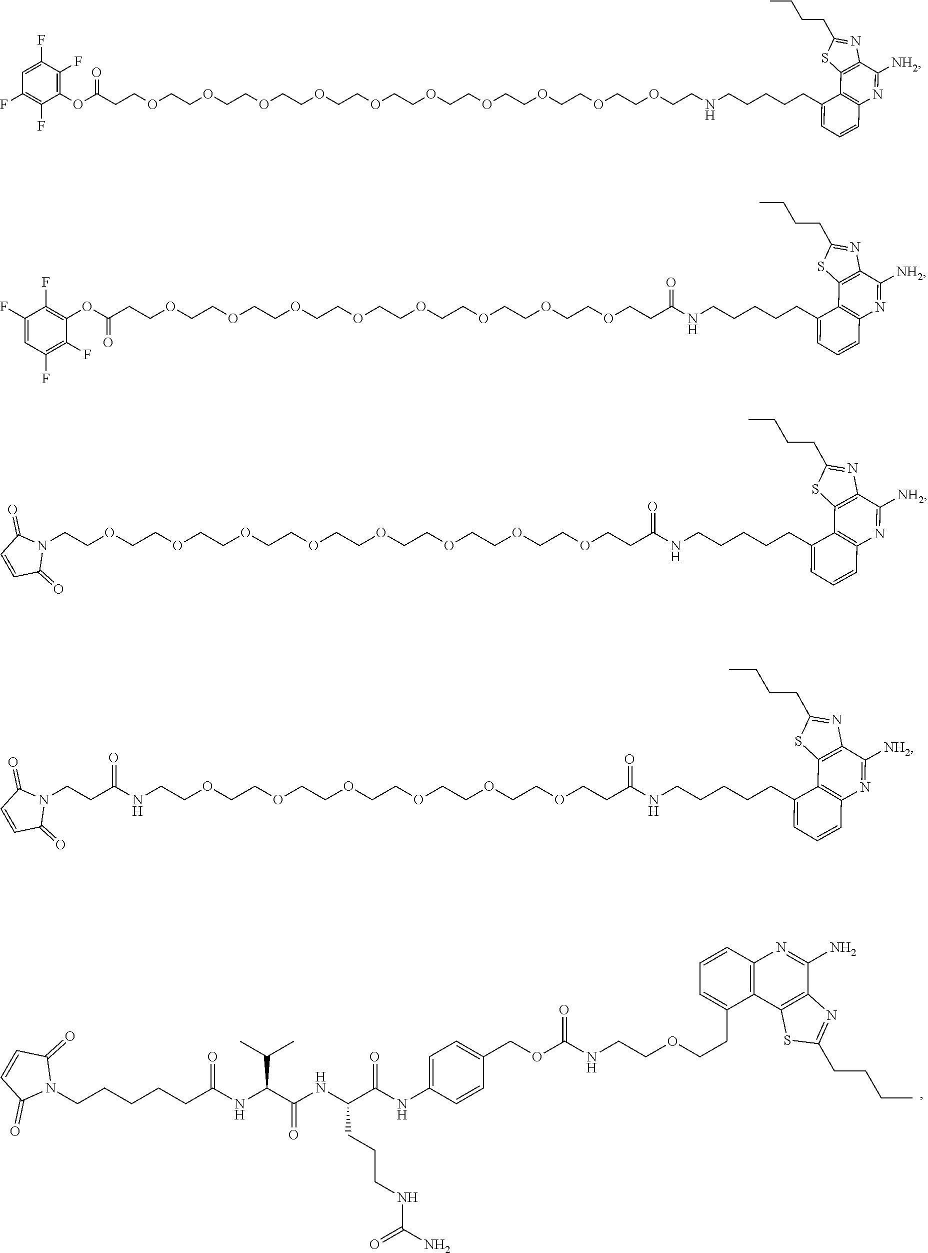 [SMILES: CCCCc1nc2c(N)nc3cccc(CCCCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCNC(=O)CCOCCOCCOCCOCCOCCOCCOCCOCCN4C(=O)C=CC4=O)c3c2s1 ; CCCCc1nc2c(N)nc3cccc(CCCCCNC(=O)CCOCCOCCOCCOCCOCCOCCNC(=O)CCN4C(=O)C=CC4=O)c3c2s1 ; CCCCc1nc2c(N)nc3cccc(CCOCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

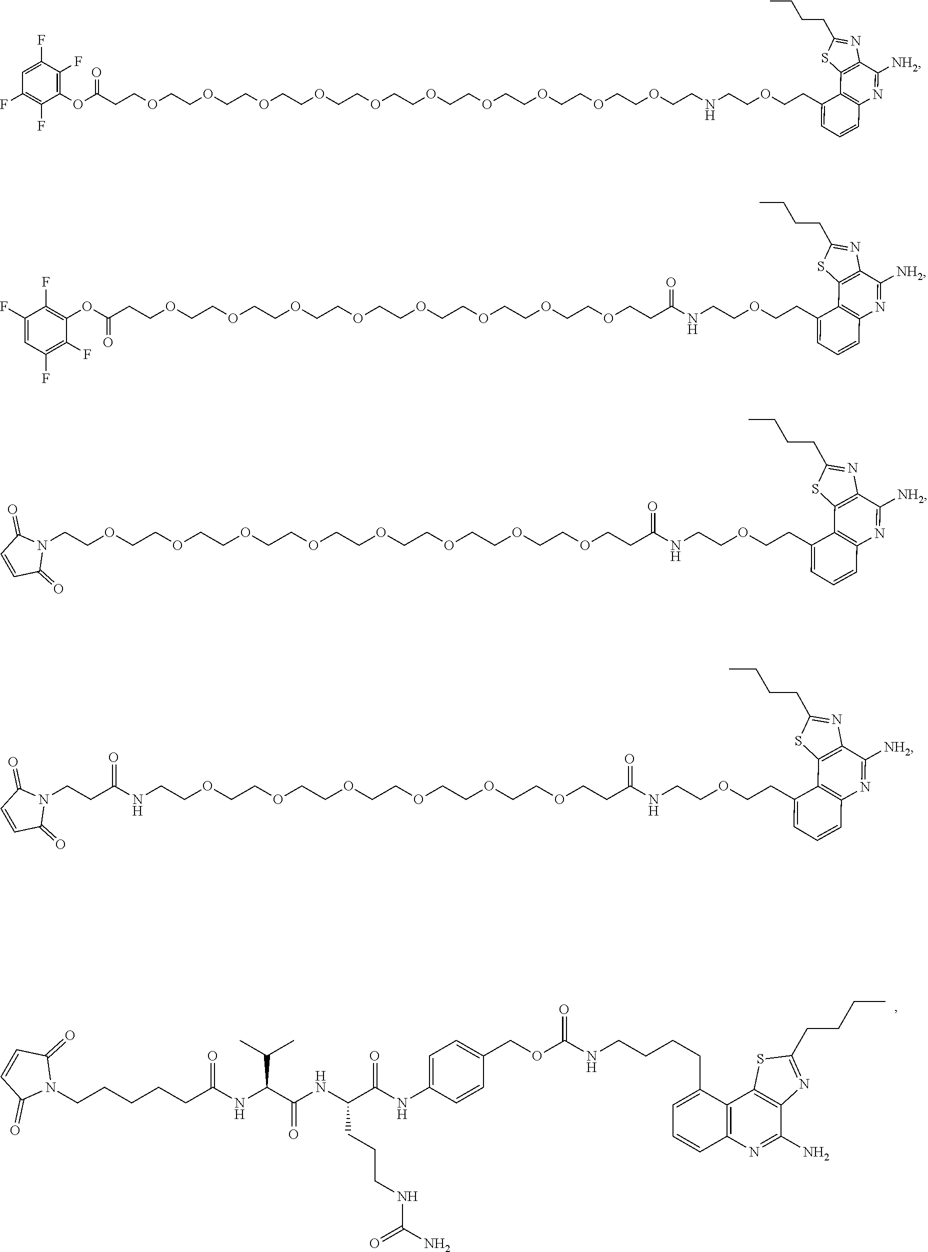 [SMILES: CCCCc1nc2c(N)nc3cccc(CCOCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCOCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCOCCNC(=O)CCOCCOCCOCCOCCOCCOCCOCCOCCN4C(=O)C=CC4=O)c3c2s1 ; CCCCc1nc2c(N)nc3cccc(CCOCCNC(=O)CCOCCOCCOCCOCCOCCOCCNC(=O)CCN4C(=O)C=CC4=O)c3c2s1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

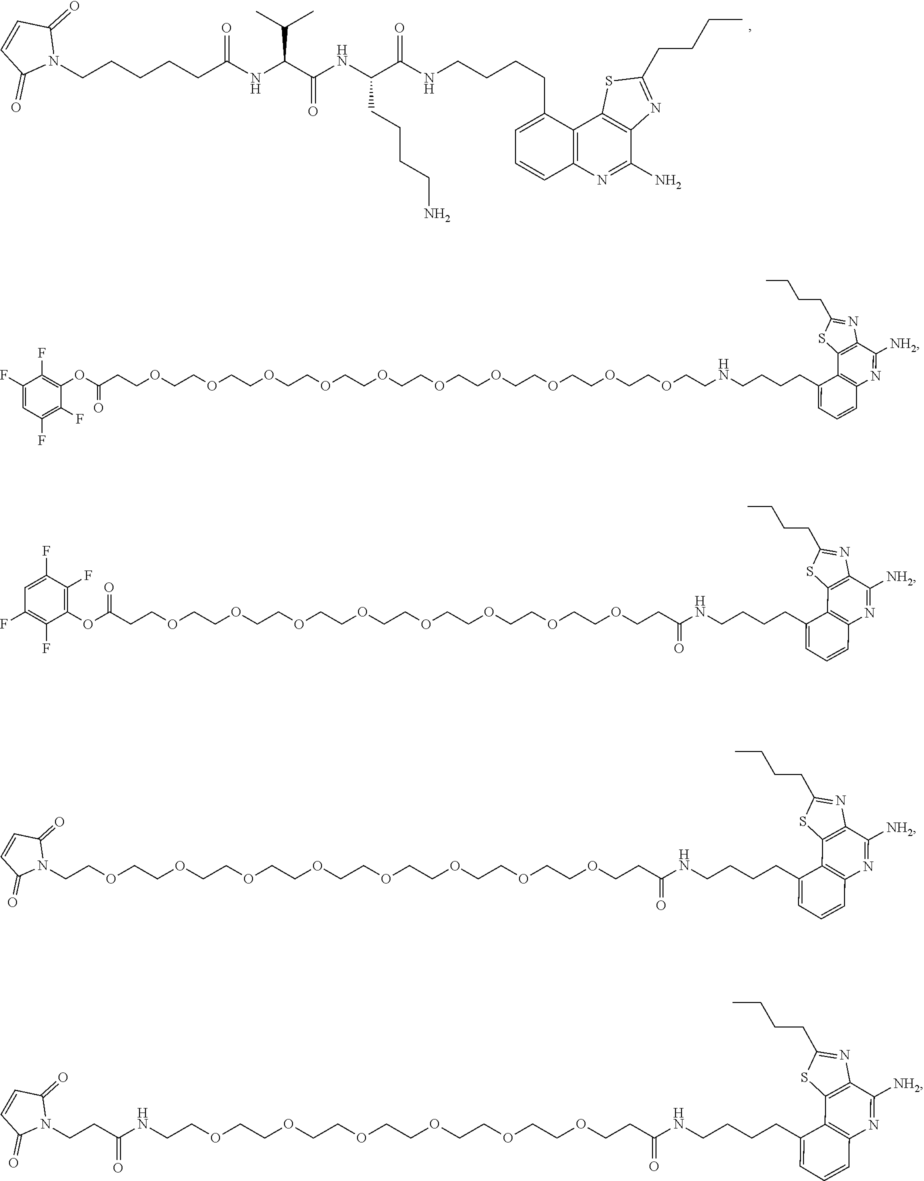 [SMILES: CCCCc1nc2c(N)nc3cccc(CCCCNC(=O)[C@H](CCCCN)NC(=O)[C@@H](NC(=O)CCCCCN4C(=O)C=CC4=O)C(C)C)c3c2s1 ; CCCCc1nc2c(N)nc3cccc(CCCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCNC(=O)CCOCCOCCOCCOCCOCCOCCOCCOCCN4C(=O)C=CC4=O)c3c2s1 ; CCCCc1nc2c(N)nc3cccc(CCCCNC(=O)CCOCCOCCOCCOCCOCCOCCNC(=O)CCN4C(=O)C=CC4=O)c3c2s1]

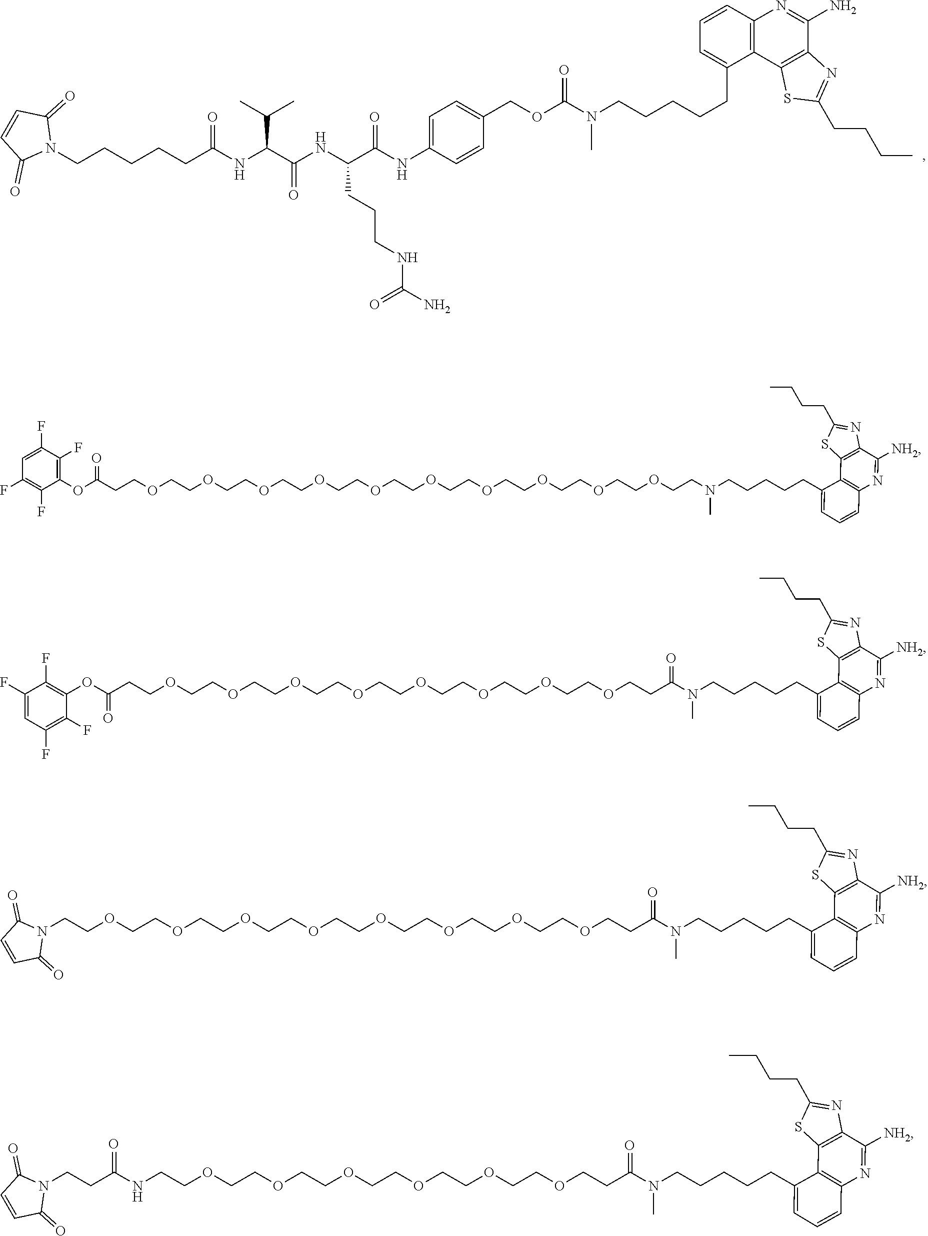 [SMILES: CCCCc1nc2c(N)nc3cccc(CCCCCN(C)C(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCN(C)CCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCCN(C)C(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCN(C)C(=O)CCOCCOCCOCCOCCOCCOCCOCCOCCN4C(=O)C=CC4=O)c3c2s1 ; CCCCc1nc2c(N)nc3cccc(CCCCCN(C)C(=O)CCOCCOCCOCCOCCOCCOCCNC(=O)CCN4C(=O)C=CC4=O)c3c2s1]

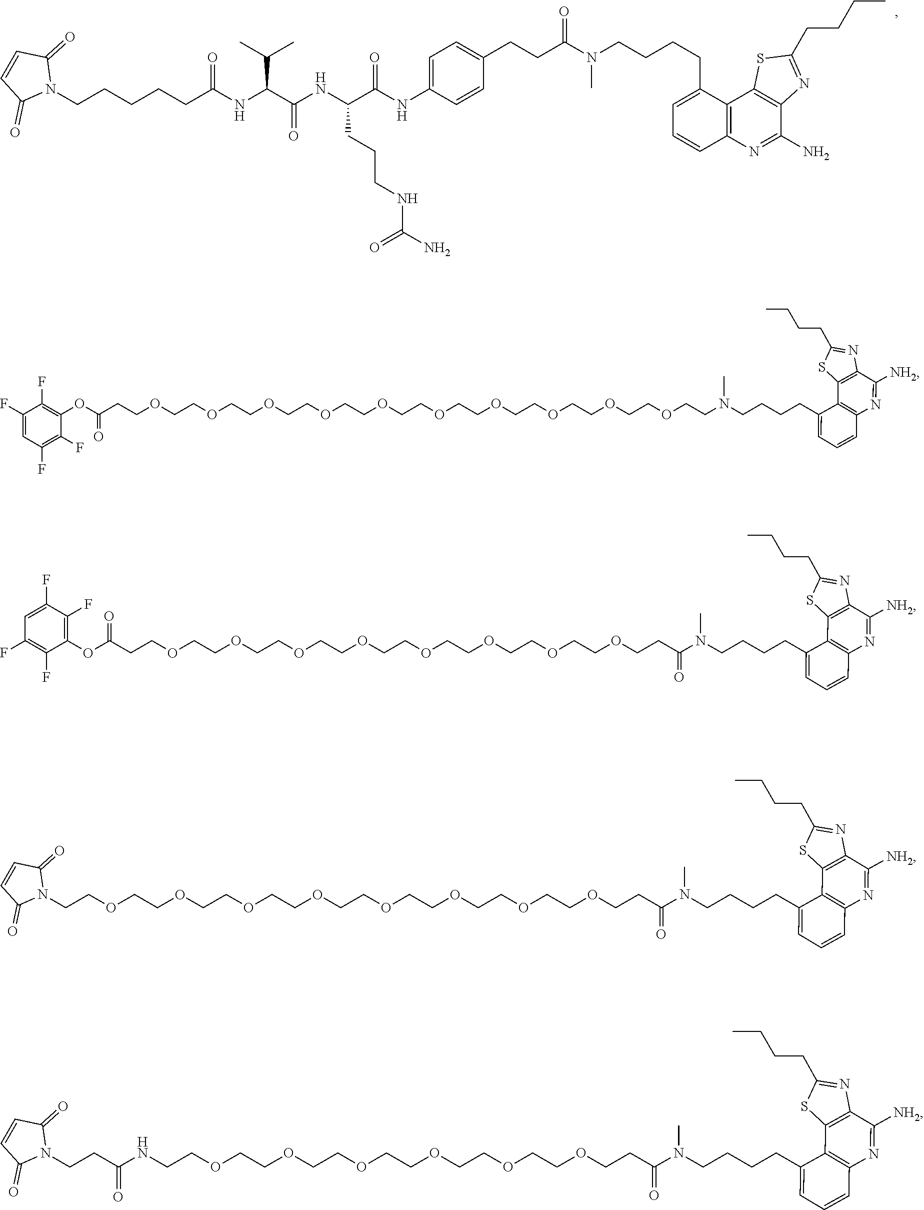 [SMILES: CCCCc1nc2c(N)nc3cccc(CCCCN(C)C(=O)CCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCN(C)CCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCN(C)C(=O)CCOCCOCCOCCOCCOCCOCCOCCOCCC(=O)Oc4c(F)c(F)cc(F)c4F)c3c2s1 ; CCCCc1nc2c(N)nc3cccc(CCCCN(C)C(=O)CCOCCOCCOCCOCCOCCOCCOCCOCCN4C(=O)C=CC4=O)c3c2s1 ; CCCCc1nc2c(N)nc3cccc(CCCCN(C)C(=O)CCOCCOCCOCCOCCOCCOCCNC(=O)CCN4C(=O)C=CC4=O)c3c2s1]

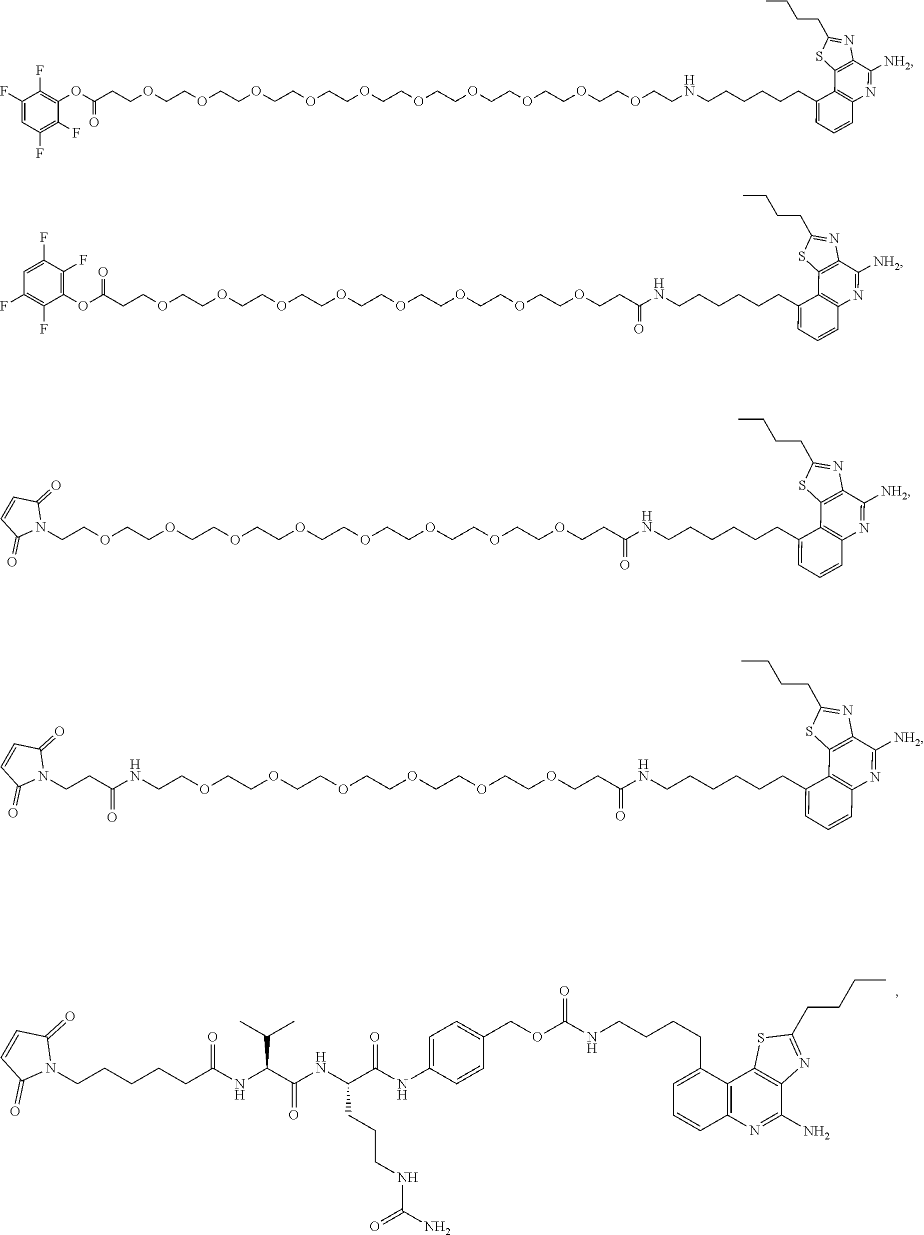 [SMILES: CCCCc1nc2c(N)nc3cccc(CCCCCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCCNC(=O)CCOCCOCCOCCOCCOCCOCCOCCOCCN4C(=O)C=CC4=O)c3c2s1 ; CCCCc1nc2c(N)nc3cccc(CCCCCCNC(=O)CCOCCOCCOCCOCCOCCOCCNC(=O)CCN4C(=O)C=CC4=O)c3c2s1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

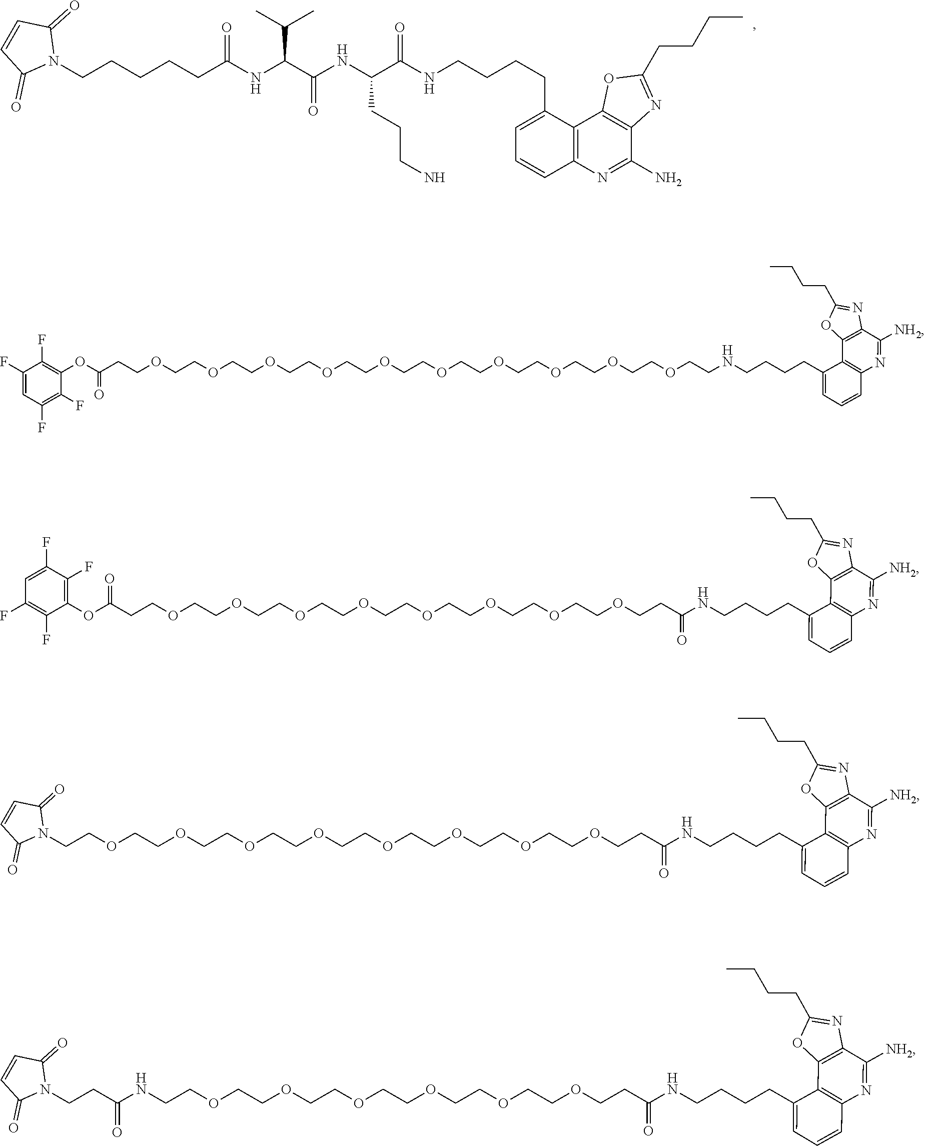 [SMILES: CCCCc1nc2c(N)nc3cccc(CCCCNC(=O)[C@H](CCCN)NC(=O)[C@@H](NC(=O)CCCCCN4C(=O)C=CC4=O)C(C)C)c3c2o1 ; CCCCc1nc2c(N)nc3cccc(CCCCNCCOCCOCCOCCOCCOCCOCCOCCOCCOCCOCCC(=O)Oc4c(F)c(F)cc(F)c4F)c3c2o1 ; CCCCc1nc2c(N)nc3cccc(CCCCNC(=O)CCOCCOCCOCCOCCOCCOCCOCCOCCC(=O)Oc4c(F)c(F)cc(F)c4F)c3c2o1 ; CCCCc1nc2c(N)nc3cccc(CCCCNC(=O)CCOCCOCCOCCOCCOCCOCCOCCOCCN4C(=O)C=CC4=O)c3c2o1 ; CCCCc1nc2c(N)nc3cccc(CCCCNC(=O)CCOCCOCCOCCOCCOCCOCCNC(=O)CCN4C(=O)C=CC4=O)c3c2o1]

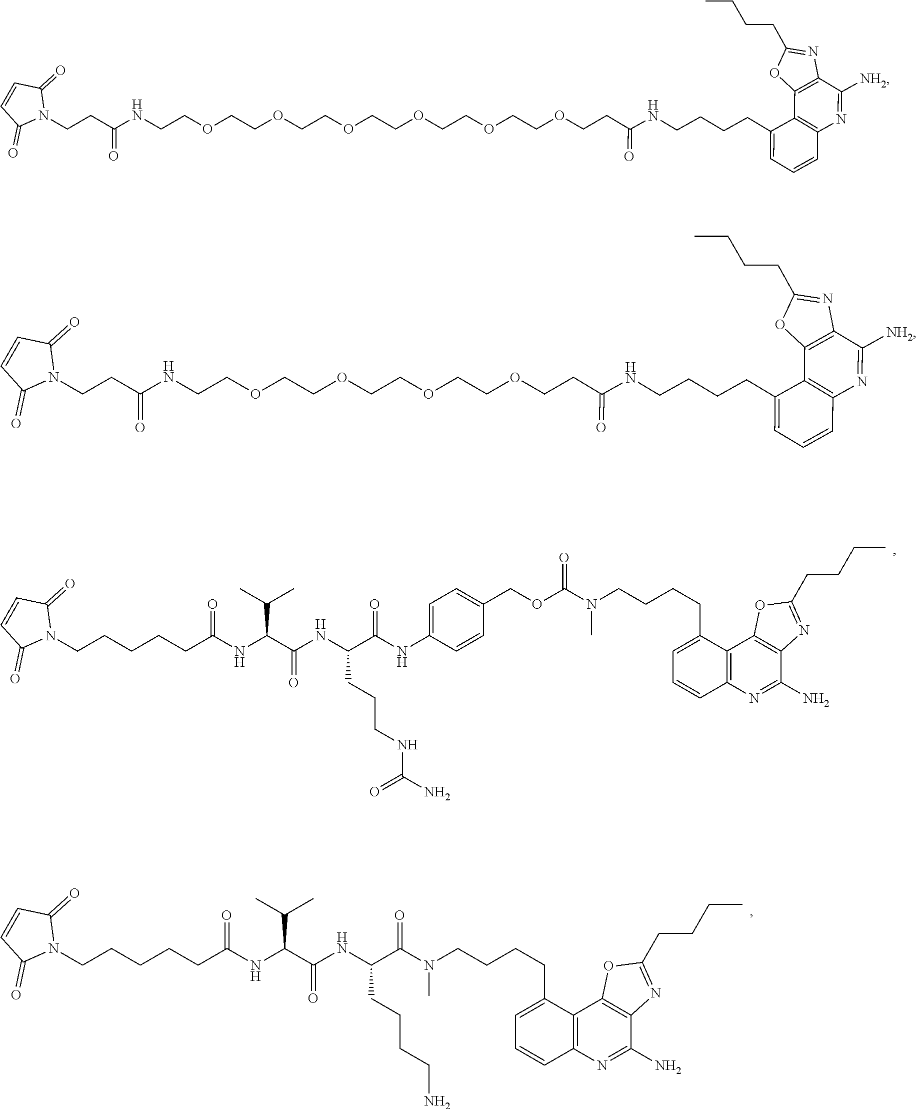 [SMILES: CCCCc1nc2c(N)nc3cccc(CCCCNC(=O)CCOCCOCCOCCOCCOCCOCCNC(=O)CCN4C(=O)C=CC4=O)c3c2o1 ; CCCCc1nc2c(N)nc3cccc(CCCCNC(=O)CCOCCOCCOCCOCCNC(=O)CCN4C(=O)C=CC4=O)c3c2o1 ; CCCCc1nc2c(N)nc3cccc(CCCCN(C)C(=O)OCc4ccc(NC(=O)[C@H](CCCNC(N)=O)NC(=O)[C@@H](NC(=O)CCCCCN5C(=O)C=CC5=O)C(C)C)cc4)c3c2o1 ; CCCCc1nc2c(N)nc3cccc(CCCCN(C)C(=O)[C@H](CCCCN)NC(=O)[C@@H](NC(=O)CCCCCN4C(=O)C=CC4=O)C(C)C)c3c2o1]

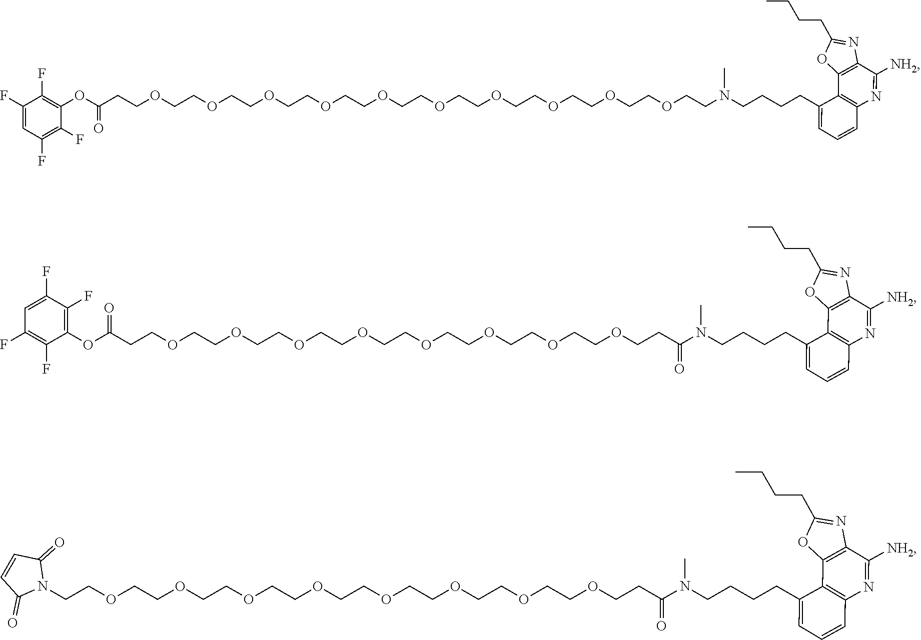 [SMILES: CCCCc1nc2c(N)nc3cccc(CCCCN(C)CCOCCOCCOCCOCCOCCOCCOCCOCCOCCOCCC(=O)Oc4c(F)c(F)cc(F)c4F)c3c2o1 ; CCCCc1nc2c(N)nc3cccc(CCCCN(C)C(=O)CCOCCOCCOCCOCCOCCOCCOCCOCCC(=O)Oc4c(F)c(F)cc(F)c4F)c3c2o1 ; CCCCc1nc2c(N)nc3cccc(CCCCN(C)C(=O)CCOCCOCCOCCOCCOCCOCCOCCOCCN4C(=O)C=CC4=O)c3c2o1]

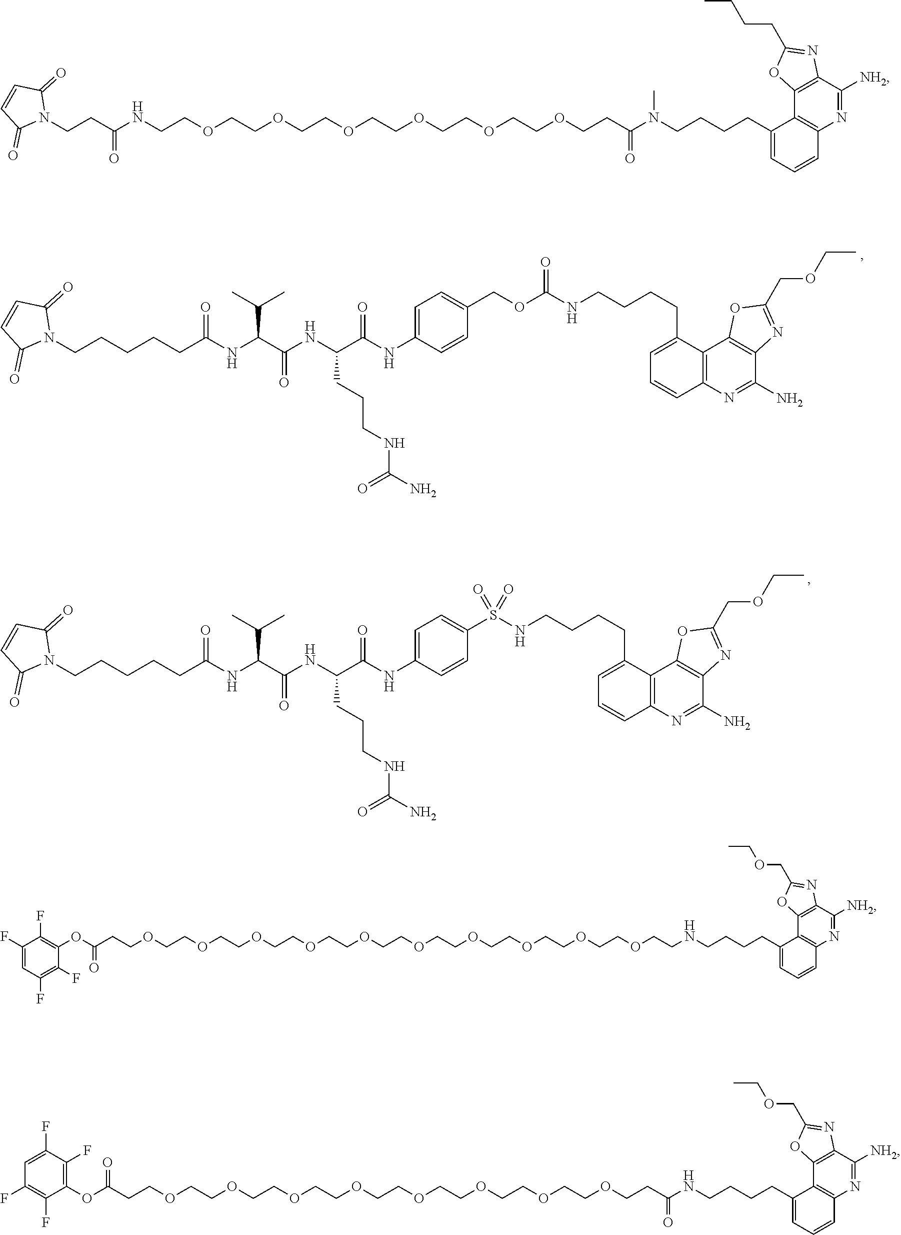

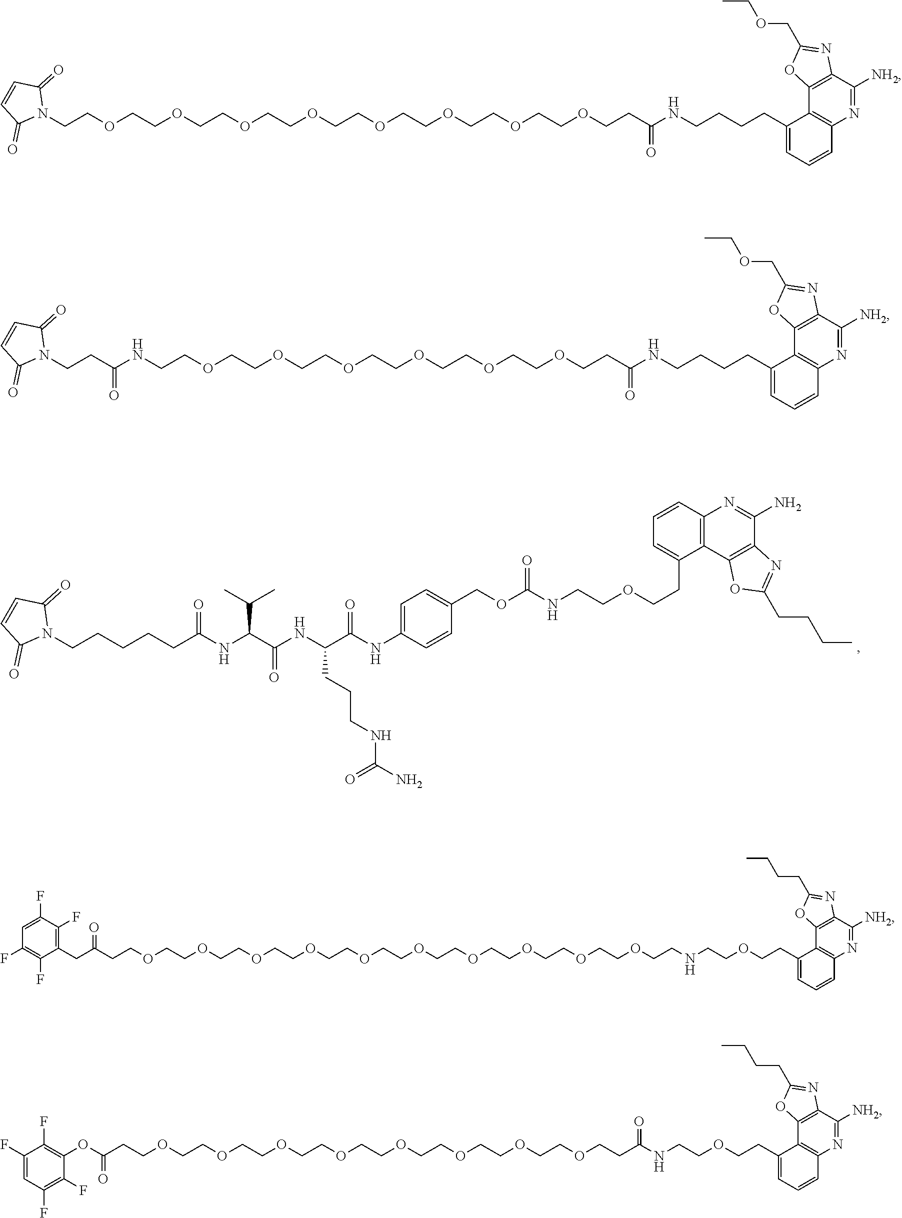 [SMILES: CCOCc1nc2c(N)nc3cccc(CCCCNC(=O)CCOCCOCCOCCOCCOCCOCCOCCOCCN4C(=O)C=CC4=O)c3c2o1 ; CCOCc1nc2c(N)nc3cccc(CCCCNC(=O)CCOCCOCCOCCOCCOCCOCCNC(=O)CCN4C(=O)C=CC4=O)c3c2o1 ; CCCCc1nc2c(N)nc3cccc(CCOCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2o1 ; CCCCc1nc2c(N)nc3cccc(CCOCCNCCOCCOCCOCCOCCOCCOCCOCCOCCOCCOCCC(=O)Cc4c(F)c(F)cc(F)c4F)c3c2o1 ; CCCCc1nc2c(N)nc3cccc(CCOCCNC(=O)CCOCCOCCOCCOCCOCCOCCOCCOCCC(=O)Oc4c(F)c(F)cc(F)c4F)c3c2o1]

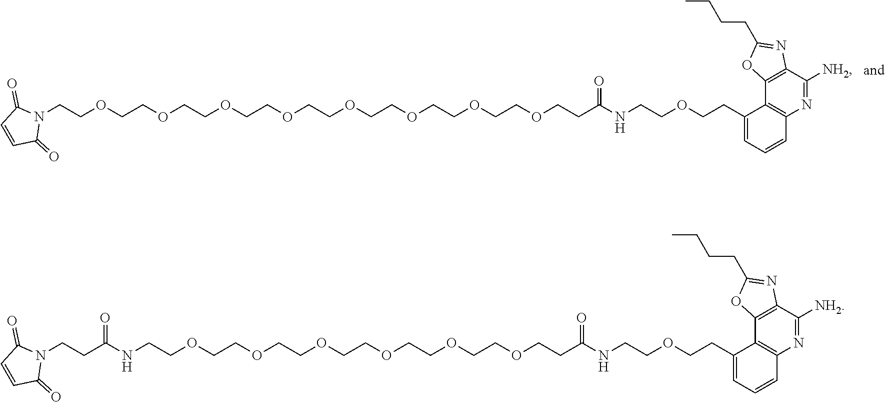 [SMILES: CCCCc1nc2c(N)nc3cccc(CCOCCNC(=O)CCOCCOCCOCCOCCOCCOCCOCCOCCN4C(=O)C=CC4=O)c3c2o1 ; CCCCc1nc2c(N)nc3cccc(CCOCCNC(=O)CCOCCOCCOCCOCCOCCOCCNC(=O)CCN4C(=O)C=CC4=O)c3c2o1]

In another aspect, the present disclosure provides a ligand-drug conjugate, comprising a ligand and a drug linked to the ligand, wherein the drug is the compound according to any one of the embodiments of the present application; preferably, the drug is linked to the ligand via a linker; preferably the ligand is an antibody or an antigen-binding fragment thereof.

In some embodiments, in the ligand-drug conjugate, or the isomer thereof, or the mixture thereof, or the pharmaceutically acceptable salt thereof described herein, n is an integer or a decimal from 2 to 8. For example, the average connection number n may be an integer or a decimal from 3 to 8. For example, the average connection number n may be an integer or a decimal from 1 to 2, 2 to 3, 3 to 4, 4 to 5, 5 to 6, 6 to 7, 7 to 8, 8 to 9, or 9 to 10.

In some embodiments, in the ligand-drug conjugate, or the isomer thereof, or the mixture thereof, or the pharmaceutically acceptable salt thereof of the present disclosure, Pc is selected from protein hormones, lectins, growth factors, antibodies, or other molecules capable of binding to cells, receptors, and/or antigens.

In some embodiments, in the ligand-drug conjugate, or the isomer thereof, or the mixture thereof, or the pharmaceutically acceptable salt thereof described herein, Pc is an antibody or an antigen-binding fragment thereof.

In some embodiments, in the ligand-drug conjugate, or the isomer thereof, or the mixture thereof, or the pharmaceutically acceptable salt thereof described herein, Pc is a chimeric antibody, a humanized antibody, and a fully human antibody.

In some embodiments, in the ligand-drug conjugate, or the isomer thereof, or the mixture thereof, or the pharmaceutically acceptable salt thereof of the present disclosure, Pc is an antibody that targets the following target points: 5T4, AGS-16, ANGPTL4, ApoE, CD19, CTGF, CXCR5, FGF2, MCPT8, MFI2, MS4A7, NCA, Sema5b, SLITRK6, STC2, TGF, 0772P, 5T4, ACTA2, ADGRE1, AG-7, AIF1, AKR1C1, AKR1C2, ASLG659, Axl, B7H3, BAFF-R, BCMA, BMPR1B, BNIP3, C1QA, C1QB, CA6, CADM1, CCD79b, CCL5, CCR5, CCR7, CDllc, CD123, CD138, CD142, CD147, CD166, CD19, CD19, CD22, CD21, CD20, CD205, CD22, CD223, CD228, CD25, CD30, CD33, CD37, CD38, CD40, CD45, CD45 (PTPRC), CD46, CD47, CD49D (ITGA4), CD56, CD66e, CD70, CD71, CD72, CD74, CD79a, CD79b, CD80, CDCP1, CDH11, CD11b, CEA, CEACAM5, c-Met, COL6A3, COL7A1, CRIPTO, CSF1R, CTSD, CTSS, CXCL11, CXCL10, DDIT4, DLL3, DLL4, DR5, E16, EFNA4, EGFR, EGFRvIII, EGLN, EGLN3, EMR2, ENPP3, EpCAM, EphA2, EphB2R, ETBR, FcRH2, FcRHl, FGFR2, FGFR3, FLT3, FOLR-α, GD2, GEDA, GPC-1, GPNMB, GPR20, GZMB, HER2, HER3, HLA-DOB, HMOX1, IFI6, IFNG, IGF-1R, IGFBP3, IL10RA1, IL-13R, IL-2, IL20Ra, IL-3, IL-4, IL-6, IRTA2, KISSIR, KRT33A, LIV-1, LOX, LRP-1, LRRC15, LUM, LY64, LY6E, Ly86, LYPD3, MDP, MMP10, MMP14, MMP16, MPF, MSG783, MSLN, MUC-1, NaPi2b, Napi3b, Nectin-4, Nectin-4, NOG, P2X5, pCAD, P-Cadherin, PDGFRA, PDK1, PD-L1, PFKFB3, PGF, PGK1, PIK3AP1, PIK3CD, PLOD2, PSCA, PSCAhlg, PSMA, PSMA, PTK7, P-Cadherin, RNF43, NaPi2b, ROR1, ROR2, SERPINE1, SLC39A6, SLTRK6, STAT1, STEAP1, STEAP2, TCF4, TENB2, TGFB1, TGFB2, TGFBR1, TNFRSF21, TNFSF9, Trop-2, TrpM4, Tyro7, UPK1B, VEGFA, WNT5A, epidermal growth factors, brevican, mesothelin, sodium phosphate cotransporter 2B, Claudin 18.2, endothelin receptors, mucins (e.g., mucin 1 and mucin 16), guanylate cyclase C, integrin a4p7, integrin a5p6, trophoblast glycoprotein, and tissue factors.

In some embodiments, in the ligand-drug conjugate, or the isomer thereof, or the mixture thereof, or the pharmaceutically acceptable salt thereof of the present disclosure, Pc is an antibody or an antigen-binding fragment thereof that targets GPC3, Trop2, and HER2; preferably, the antibody is selected from: trastuzumab, pertuzumab, sacituzumab, and codrituzumab.

In the present application, CDRs of the antibody or the antigen-binding fragment thereof may be defined according to Kabat.

In the present application, the antibody or the antigen-binding fragment thereof may comprise LCDR1-LCDR3 and HCDR1-HCDR3, wherein the LCDR1 may comprise the amino acid sequence set forth in any one of SEQ ID NOs: 1-4, the LCDR2 may comprise the amino acid sequence set forth in any one of SEQ ID NOs: 5-8, the LCDR3 may comprise the amino acid sequence set forth in any one of SEQ ID NOs: 9-12, the HCDR1 may comprise the amino acid sequence set forth in any one of SEQ ID NOs: 13-16, the HCDR2 may comprise the amino acid sequence set forth in any one of SEQ ID NOs: 17-20, and the HCDR3 may comprise the amino acid sequence set forth in any one of SEQ ID NOs: 21-24.

For example, the antibody or the antigen-binding fragment thereof described herein comprises the same LCDR1-LCDR3 and HCDR1-HCDR3 as trastuzumab, wherein the LCDR1 may comprise the amino acid sequence set forth in SEQ ID NO: 1, the LCDR2 may comprise the amino acid sequence set forth in SEQ ID NO: 5, the LCDR3 may comprise the amino acid sequence set forth in SEQ ID NO: 9, the HCDR1 may comprise the amino acid sequence set forth in SEQ ID NO: 13, the HCDR2 may comprise the amino acid sequence set forth in SEQ ID NO: 17, and the HCDR3 may comprise the amino acid sequence set forth in SEQ ID NO: 21.

For example, the antibody or the antigen-binding fragment thereof described herein may comprise the same LCDR1-LCDR3 and HCDR1-HCDR3 as pertuzumab, wherein the LCDR1 may comprise the amino acid sequence set forth in SEQ ID NO: 2, the LCDR2 may comprise the amino acid sequence set forth in SEQ ID NO: 6, the LCDR3 may comprise the amino acid sequence set forth in SEQ ID NO: 10, the HCDR1 may comprise the amino acid sequence set forth in SEQ ID NO: 14, the HCDR2 may comprise the amino acid sequence set forth in SEQ ID NO: 18, and the HCDR3 may comprise the amino acid sequence set forth in SEQ ID NO: 22.

For example, the antibody or the antigen-binding fragment thereof described herein may comprise the same LCDR1-LCDR3 and HCDR1-HCDR3 as sacituzumab, wherein the LCDR1 may comprise the amino acid sequence set forth in SEQ ID NO: 3, the LCDR2 may comprise the amino acid sequence set forth in SEQ ID NO: 7, the LCDR3 may comprise the amino acid sequence set forth in SEQ ID NO: 11, the HCDR1 may comprise the amino acid sequence set forth in SEQ ID NO: 15, the HCDR2 may comprise the amino acid sequence set forth in SEQ ID NO: 19, and the HCDR3 may comprise the amino acid sequence set forth in SEQ ID NO: 23.

For example, the antibody or the antigen-binding fragment thereof described herein may comprise the same LCDR1-LCDR3 and HCDR1-HCDR3 as codrituzumab, wherein the LCDR1 may comprise the amino acid sequence set forth in SEQ ID NO: 4, the LCDR2 may comprise the amino acid sequence set forth in SEQ ID NO: 8, the LCDR3 may comprise the amino acid sequence set forth in SEQ ID NO: 12, the HCDR1 may comprise the amino acid sequence set forth in SEQ ID NO: 16, the HCDR2 may comprise the amino acid sequence set forth in SEQ ID NO: 20, and the HCDR3 may comprise the amino acid sequence set forth in SEQ ID NO: 24.

In the present application, the antibody or the antigen-binding fragment thereof may comprise a light chain variable region VL and a heavy chain variable region VH, wherein the VL may comprise the amino acid sequence set forth in any one of SEQ ID NOs: 25-28, and the VH may comprise the amino acid sequence set forth in any one of SEQ ID NOs: 29-32.

For example, the antibody or the antigen-binding fragment thereof described herein may comprise the same light chain variable region VL and heavy chain variable region VH as trastuzumab, wherein the VL may comprise the amino acid sequence set forth in SEQ ID NO: 25, and the VH may comprise the amino acid sequence set forth in SEQ ID NO: 29.

For example, the antibody or the antigen-binding fragment thereof described herein may comprise the same light chain variable region VL and heavy chain variable region VH as pertuzumab, wherein the VL may comprise the amino acid sequence set forth in SEQ ID NO: 26, and the VH may comprise the amino acid sequence set forth in SEQ ID NO: 30.

For example, the antibody or the antigen-binding fragment thereof described herein may comprise the same light chain variable region VL and heavy chain variable region VH as sacituzumab, wherein the VL may comprise the amino acid sequence set forth in SEQ ID NO: 27, and the VH may comprise the amino acid sequence set forth in SEQ ID NO: 31.

For example, the antibody or the antigen-binding fragment thereof described herein may comprise the same light chain variable region VL and heavy chain variable region VH as codrituzumab, wherein the VL may comprise the amino acid sequence set forth in SEQ ID NO: 28, and the VH may comprise the amino acid sequence set forth in SEQ ID NO: 32.

In the present application, the antibody or the antigen-binding fragment thereof may comprise an antibody light chain and an antibody heavy chain, wherein the light chain may comprise the amino acid sequence set forth in any one of SEQ ID NOs: 33-36, and the heavy chain may comprise the amino acid sequence set forth in any one of SEQ ID NOs: 37-40.

For example, the antibody or the antigen-binding fragment thereof described herein may comprise the same antibody light chain and antibody heavy chain as trastuzumab, wherein the light chain may comprise the amino acid sequence set forth in SEQ ID NO: 33, and the heavy chain may comprise the amino acid sequence set forth in SEQ ID NO: 37.

For example, the antibody or the antigen-binding fragment thereof described herein may comprise the same antibody light chain and antibody heavy chain as pertuzumab, wherein the light chain may comprise the amino acid sequence set forth in SEQ ID NO: 34, and the heavy chain may comprise the amino acid sequence set forth in SEQ ID NO: 38.

For example, the antibody or the antigen-binding fragment thereof described herein may comprise the same antibody light chain and antibody heavy chain as sacituzumab, wherein the light chain may comprise the amino acid sequence set forth in SEQ ID NO: 35, and the heavy chain may comprise the amino acid sequence set forth in SEQ ID NO: 39.

For example, the antibody or the antigen-binding fragment thereof described herein may comprise the same antibody light chain and antibody heavy chain as codrituzumab, wherein the light chain may comprise the amino acid sequence set forth in SEQ ID NO: 36, and the heavy chain may comprise the amino acid sequence set forth in SEQ ID NO: 40.

In certain preferred embodiments of the present disclosure, the present disclosure provides a compound of formula (A) below, or a tautomer, an enantiomer or a diastereoisomer thereof, or a mixture of isomers thereof, or a pharmaceutically acceptable salt or solvate thereof:

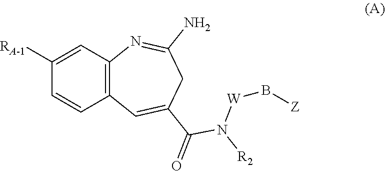

wherein
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—, and preferably, Lx is selected from single bond, —C(O)NH—, or —NHC(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein
each R_{A2-2} is independently selected from C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆ alkyl), or —N(C₁-C₆ alkyl)₂;
R₂ is selected from —C₁-C₆ alkyl, —C₁-C₆ alkoxy, —C₁-C₆ alkylenehydroxy, or halogenated C₁-C₆ alkyl;
W is selected from —C₁-C₆ alkylene- and —O—C₁-C₆ alkylene-;
B is phenyl, and is optionally substituted with one or more substituents selected from: halogen, C₁-C₆ alkyl, halogenated C₁-C₆ alkyl, and C₁-C₆ alkoxy; and
Z is —NH(R_{Z-1}), R_{Z-1} being selected from H, —C₁-C₆ alkyl, and halogenated C₁-C₆ alkyl. In a particularly preferred embodiment, the present disclosure provides a compound of formula (A) below, or a tautomer, an enantiomer or a diastereoisomer thereof, or a mixture of isomers thereof, or a pharmaceutically acceptable salt or solvate thereof selected from:

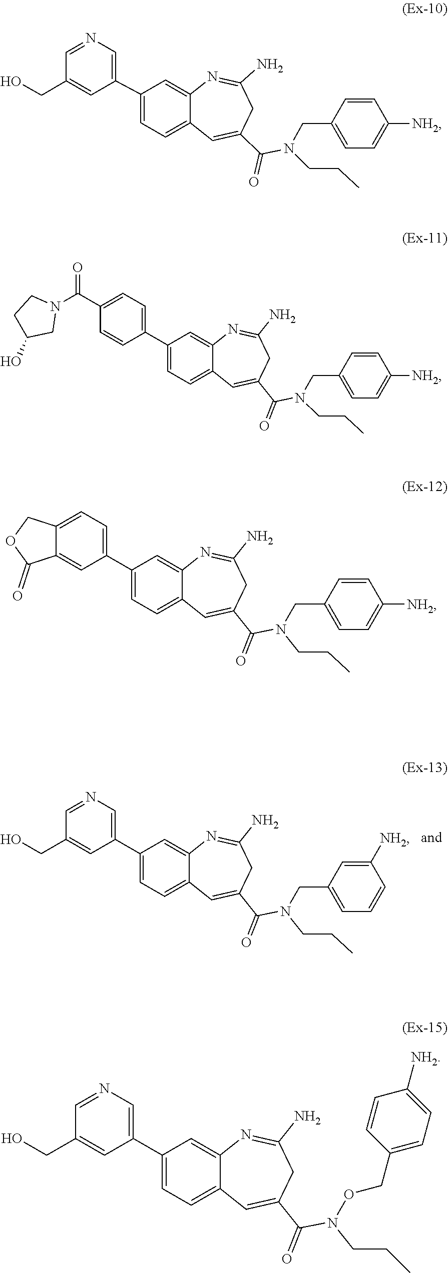 [SMILES: CCCN(Cc1ccc(N)cc1)C(=O)C1=Cc2ccc(-c3cncc(CO)c3)cc2N=C(N)C1 ; CCCN(Cc1ccc(N)cc1)C(=O)C1=Cc2ccc(-c3ccc(C(=O)N4CC[C@@H](O)C4)cc3)cc2N=C(N)C1 ; CCCN(Cc1ccc(N)cc1)C(=O)C1=Cc2ccc(-c3ccc4c(c3)C(=O)OC4)cc2N=C(N)C1 ; CCCN(Cc1cccc(N)c1)C(=O)C1=Cc2ccc(-c3cncc(CO)c3)cc2N=C(N)C1 ; "and" ; CCCN(OCc1ccc(N)cc1)C(=O)C1=Cc2ccc(-c3cncc(CO)c3)cc2N=C(N)C1]

In certain preferred embodiments of the present disclosure, the present disclosure provides a compound of formula (A-i) below, or a tautomer, an enantiomer or a diastereoisomer thereof, or a mixture of isomers thereof, or a pharmaceutically acceptable salt or solvate thereof:

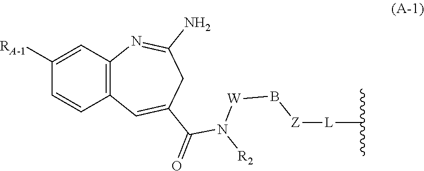

wherein R_{A-1}, W, B, Z, and R₂ are as defined above; L is a linker unit, preferably the linker unit as defined in any one of claims 27-31; the wavy line represents being linked to the ligand via the nitrogen atom or the carbon atom on the L group.

In a more preferred embodiment, the linker unit L is selected from:

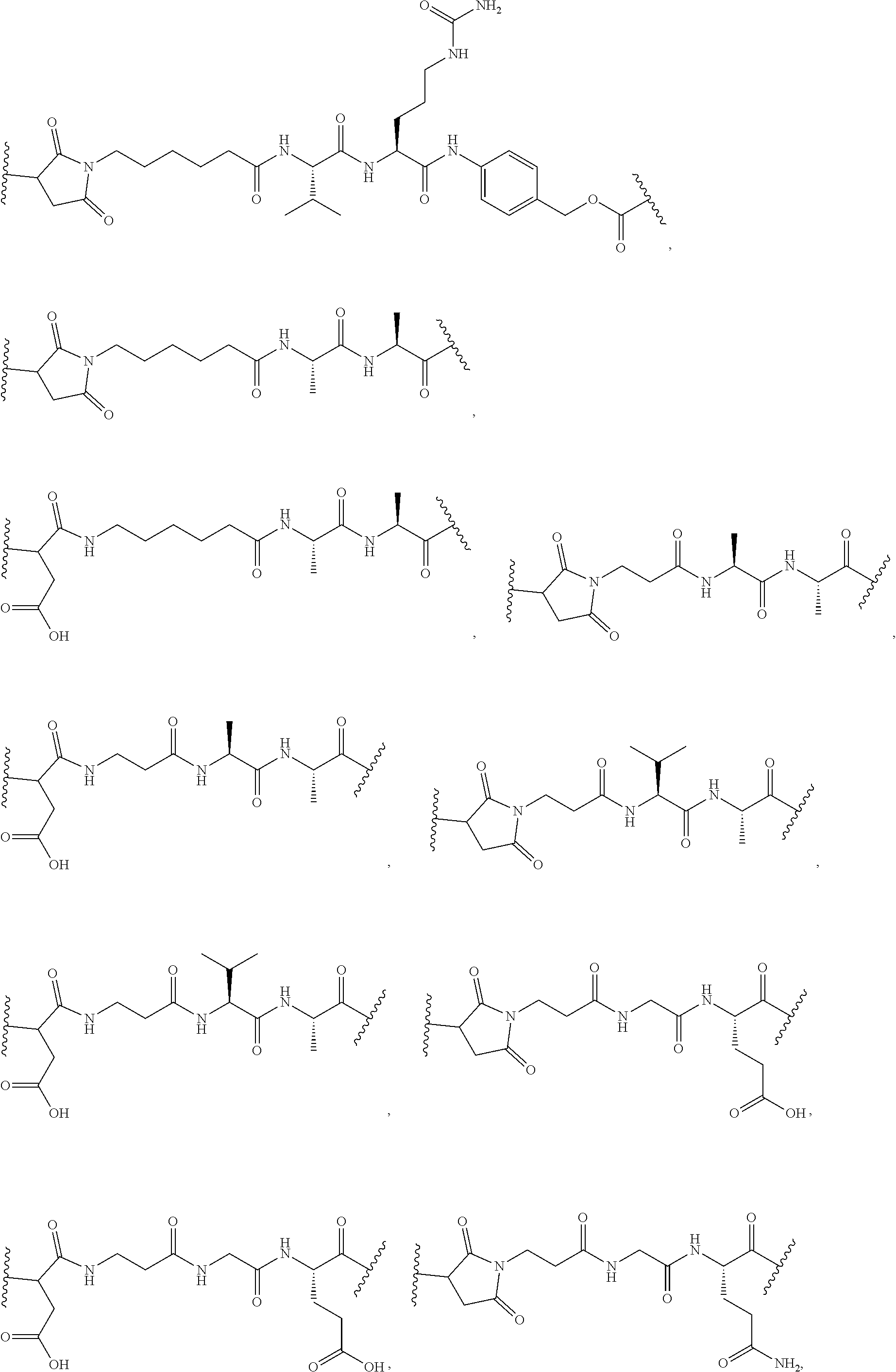

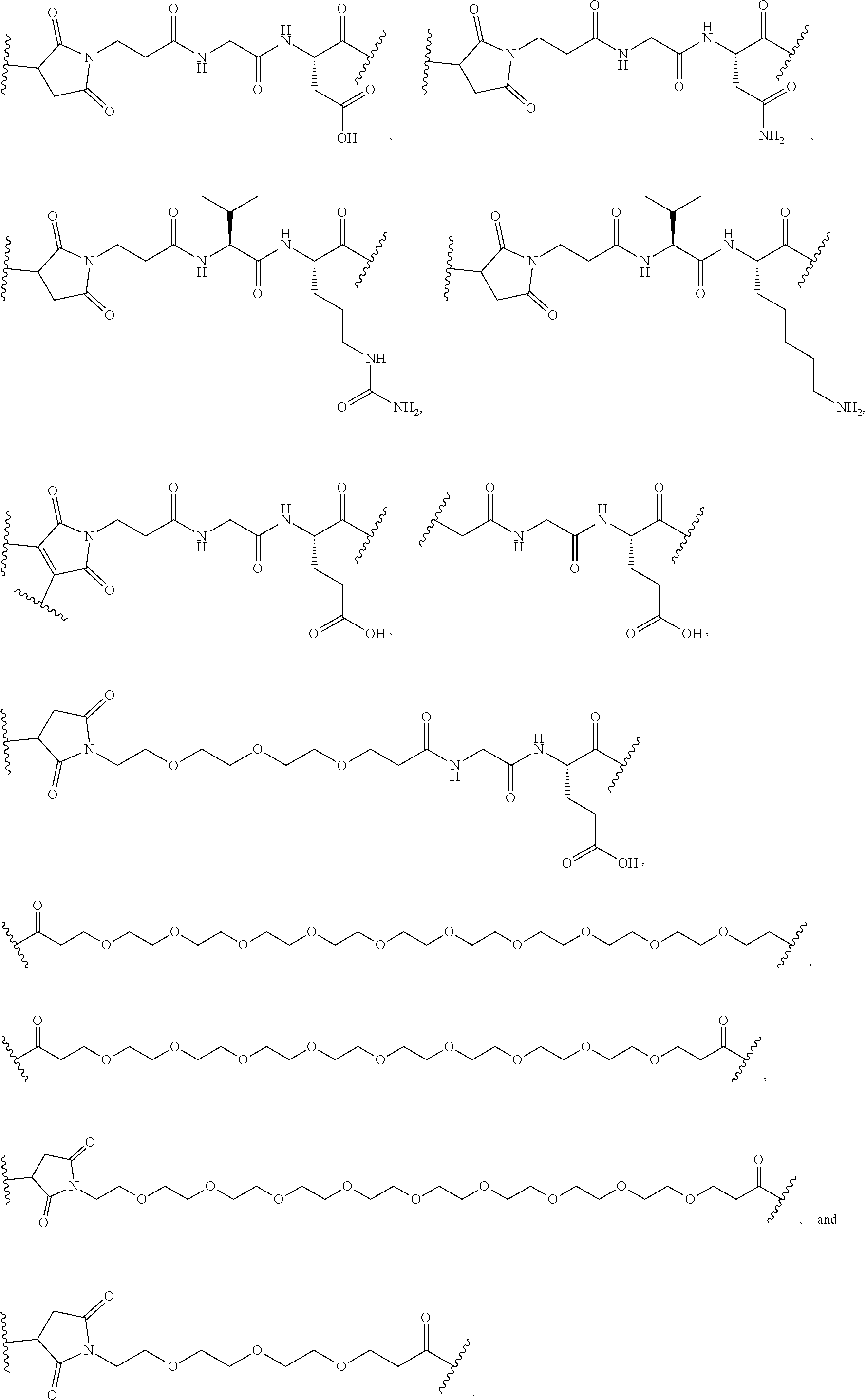

In a particularly preferred embodiment, the compound of the present disclosure is selected from the following compounds, or tautomers, enantiomers or diastereoisomers thereof, or mixtures of isomers thereof, or pharmaceutically acceptable salts or solvates thereof:

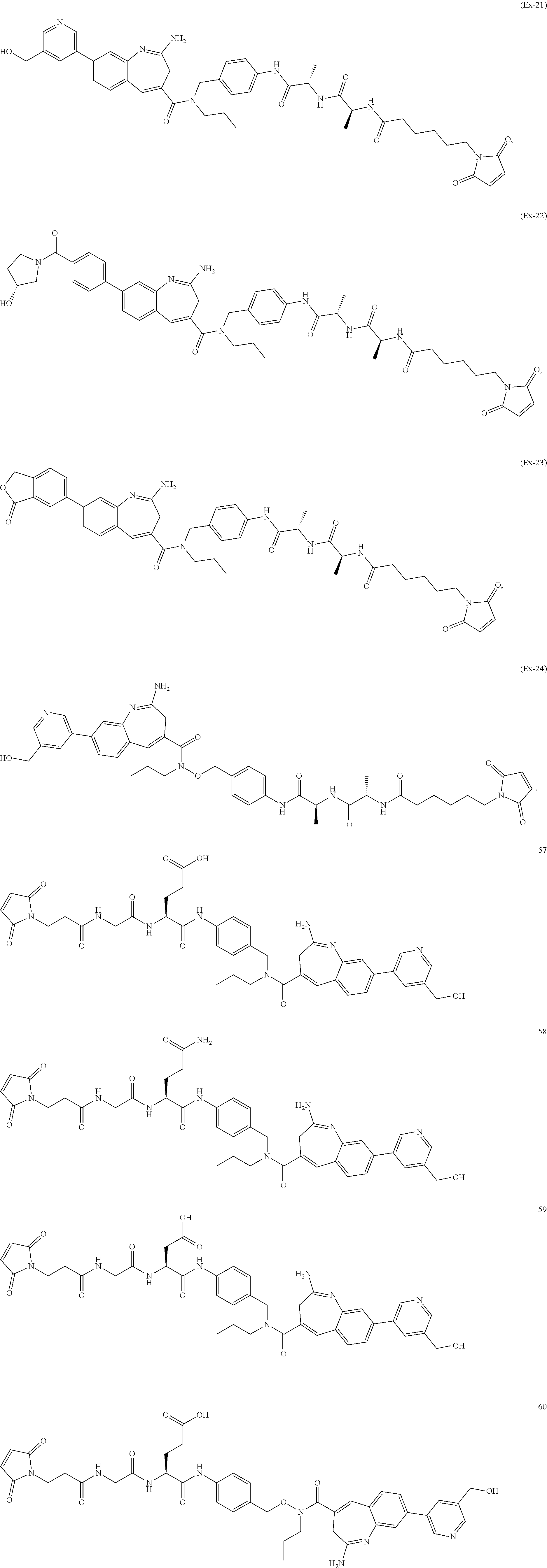

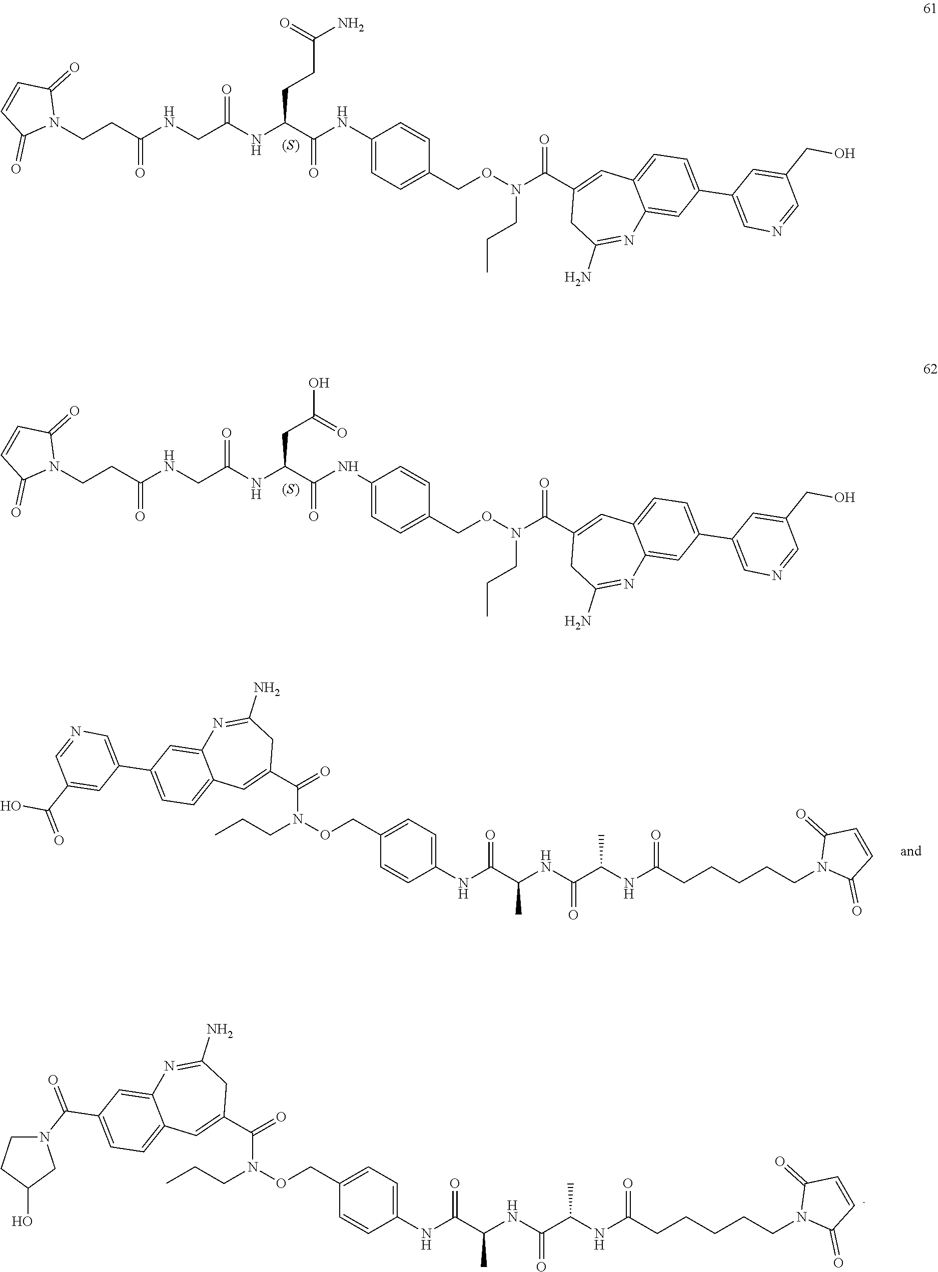

In certain preferred embodiments of the present disclosure, the present disclosure provides a ligand-drug conjugate of formula (A-2) below, or a tautomer, an enantiomer or a diastereoisomer thereof, or a mixture of isomers thereof, or a pharmaceutically acceptable salt or solvate thereof:

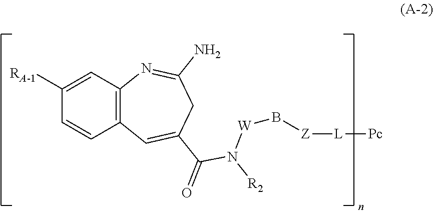

wherein R_{A-1}, W, B, Z, and R₂ are as defined above; L is the linker unit as defined above; n is an integer or a decimal from 1 to 10, preferably an integer or a decimal from 2 to 8; Pc is a ligand, preferably an antibody or an antigen-binding fragment thereof.

In a more preferred embodiment, Pc is an antibody or an antigen-binding fragment thereof that targets GPC3, Trop2, and HER2; preferably, the antibody is selected from: trastuzumab, pertuzumab, sacituzumab, and codrituzumab.

In certain preferred embodiments of the present disclosure, the present disclosure provides a compound of formula (B) below, or a tautomer, an enantiomer or a diastereoisomer thereof, or a mixture of isomers thereof, or a pharmaceutically acceptable salt or solvate thereof:

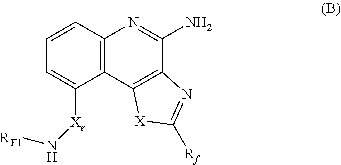

wherein
R_{Y1} is selected from hydrogen and —C₁-C₆ alkyl;
Xₑ is selected from C₁-C₈alkylene, preferably C₁-C₆ alkylene, wherein one or more carbon atoms in the C₁-C₈ alkylene and the C₁-C₆ alkylene may be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
X is selected from —O— or —S—; and
R_{f} is selected from C₁-C₆ alkyl, wherein one or more carbon atoms in the C₁-C₆ alkyl may be optionally replaced by a heteroatom selected from N, O, or S, and the C₁-C₆ alkyl is optionally substituted with one or more substituents selected from: —NH(C₁-C₆ alkyl), —N(C₁-C₆ alkyl)₂, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, —C₁-C₆ alkyl-NH(C₁-C₆ alkyl), or —C₁-C₆ alkyl-N(C₁-C₆ alkyl)₂.

In a more preferred embodiment, the present disclosure provides a compound of formula (B) below, or a tautomer, an enantiomer or a diastereoisomer thereof, or a mixture of isomers thereof, or a pharmaceutically acceptable salt or solvate thereof selected from:

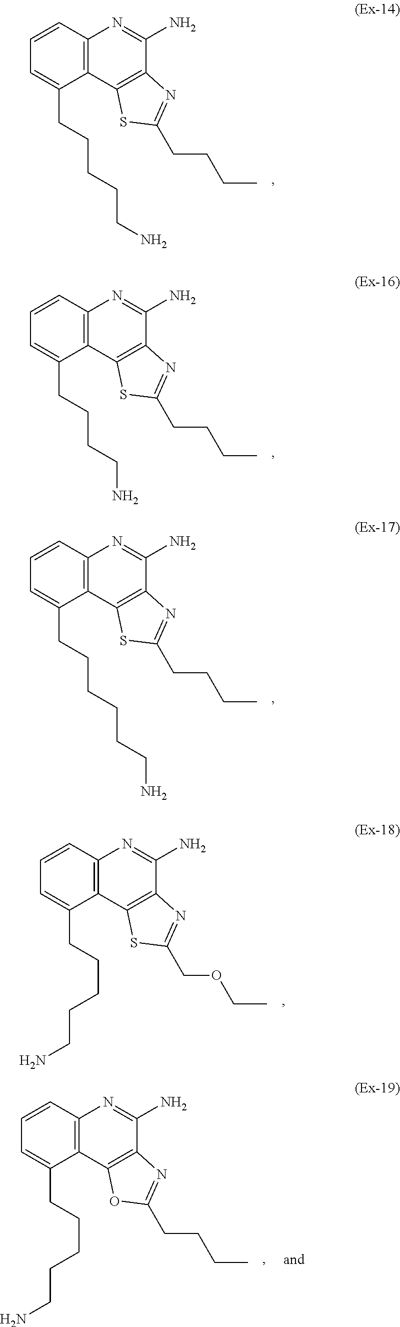

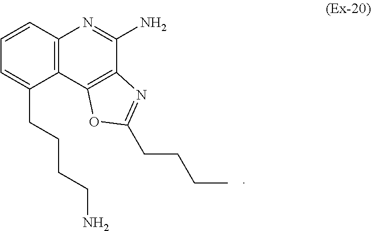 [SMILES: CCCCc1nc2c(N)nc3cccc(CCCCN)c3c2o1]

In certain preferred embodiments of the present disclosure, the present disclosure provides a compound of formula (B-1) below, or a tautomer, an enantiomer or a diastereoisomer thereof, or a mixture of isomers thereof, or a pharmaceutically acceptable salt or solvate thereof:

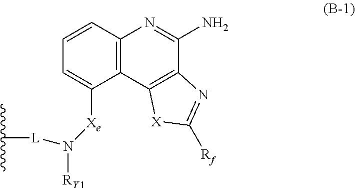

wherein X, Xₑ, R_{f}, and R_{Y1} are as defined above; L is a linker unit, preferably the linker unit as defined in any one of claims 27-31; the wavy line represents being linked to the ligand via the nitrogen atom or the carbon atom on the L group.

In a more preferred embodiment, the linker unit L is selected from:

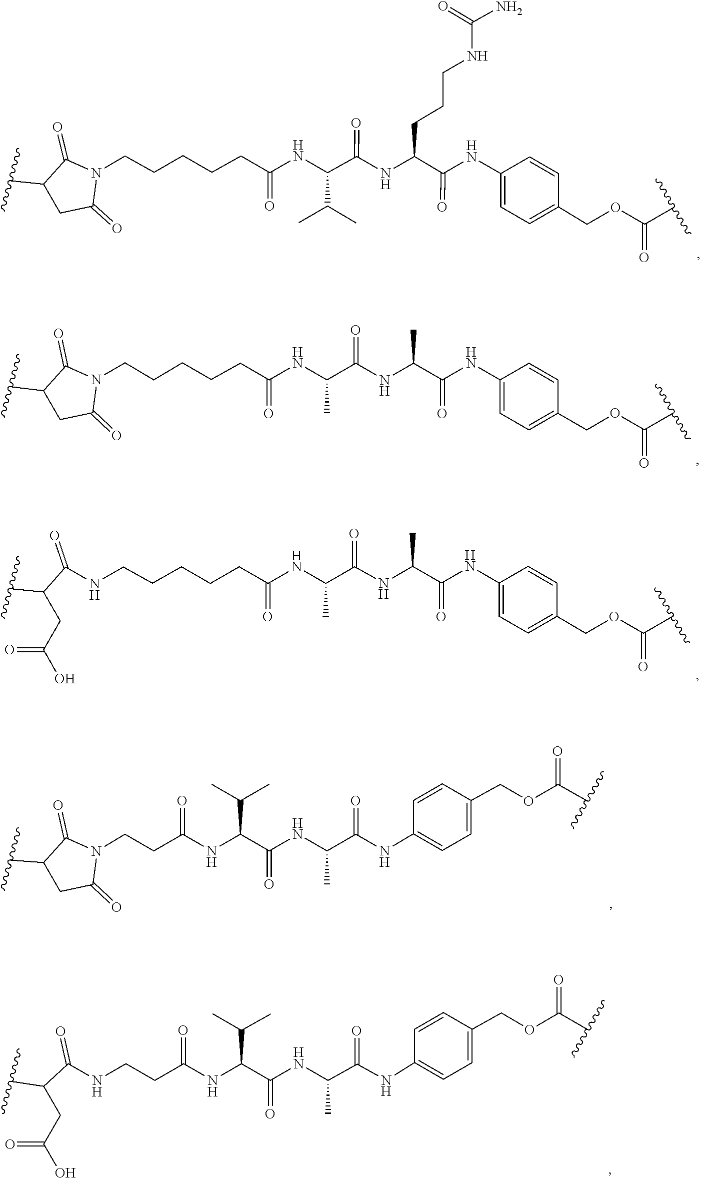 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)OCc1ccc(NC(=O)[C@H](C)NC(=O)[C@H](C)NC(=O)CCCCCN2C(=O)CC(*)C2=O)cc1 ; *C(=O)OCc1ccc(NC(=O)[C@H](C)NC(=O)[C@H](C)NC(=O)CCCCCNC(=O)C(*)CC(=O)O)cc1 ; *C(=O)OCc1ccc(NC(=O)[C@H](C)NC(=O)[C@@H](NC(=O)CCN2C(=O)CC(*)C2=O)C(C)C)cc1 ; *C(=O)OCc1ccc(NC(=O)[C@H](C)NC(=O)[C@@H](NC(=O)CCNC(=O)C(*)CC(=O)O)C(C)C)cc1]

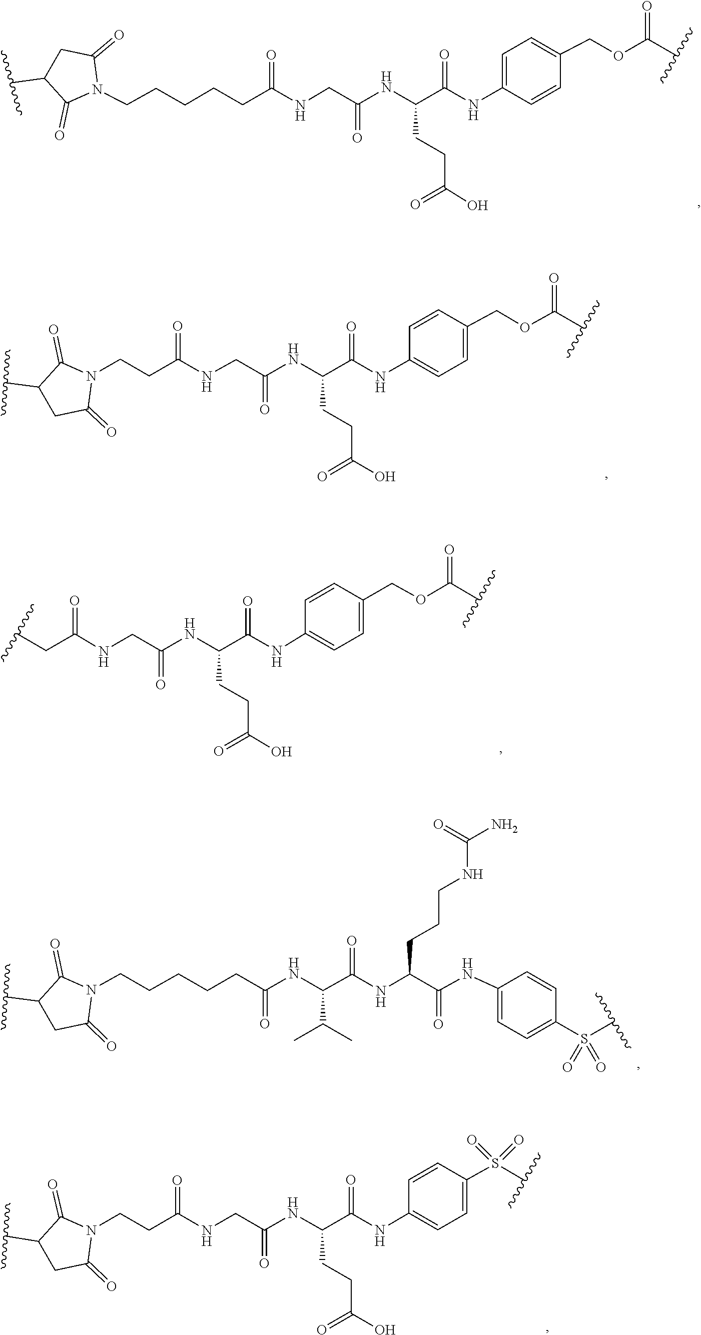 [SMILES: *C(=O)OCc1ccc(NC(=O)[C@H](CCC(=O)O)NC(=O)CNC(=O)CCCCCN2C(=O)CC(*)C2=O)cc1 ; *C(=O)OCc1ccc(NC(=O)[C@H](CCC(=O)O)NC(=O)CNC(=O)CCN2C(=O)CC(*)C2=O)cc1 ; *CC(=O)NCC(=O)N[C@@H](CCC(=O)O)C(=O)Nc1ccc(COC(*)=O)cc1 ; *C1CC(=O)N(CCCCCC(=O)N[C@H](C(=O)N[C@@H](CCCNC(N)=O)C(=O)Nc2ccc(S(*)(=O)=O)cc2)C(C)C)C1=O ; *C1CC(=O)N(CCC(=O)NCC(=O)N[C@@H](CCC(=O)O)C(=O)Nc2ccc(S(*)(=O)=O)cc2)C1=O]

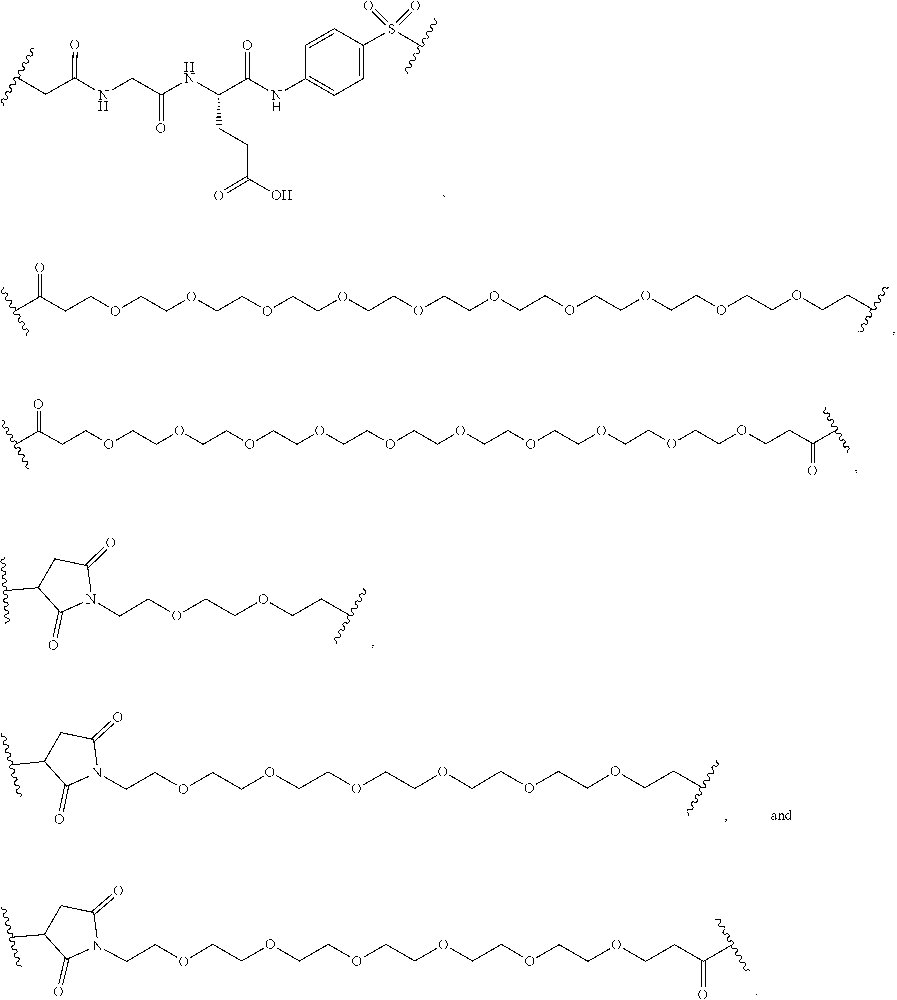 [SMILES: *CC(=O)NCC(=O)N[C@@H](CCC(=O)O)C(=O)Nc1ccc(S(*)(=O)=O)cc1 ; *CCOCCOCCOCCOCCOCCOCCOCCOCCOCCOCCC(*)=O ; *C(=O)CCOCCOCCOCCOCCOCCOCCOCCOCCOCCOCCC(*)=O ; *CCOCCOCCN1C(=O)CC(*)C1=O ; *CCOCCOCCOCCOCCOCCOCCN1C(=O)CC(*)C1=O ; *C(=O)CCOCCOCCOCCOCCOCCOCCN1C(=O)CC(*)C1=O]

For example, L is selected from

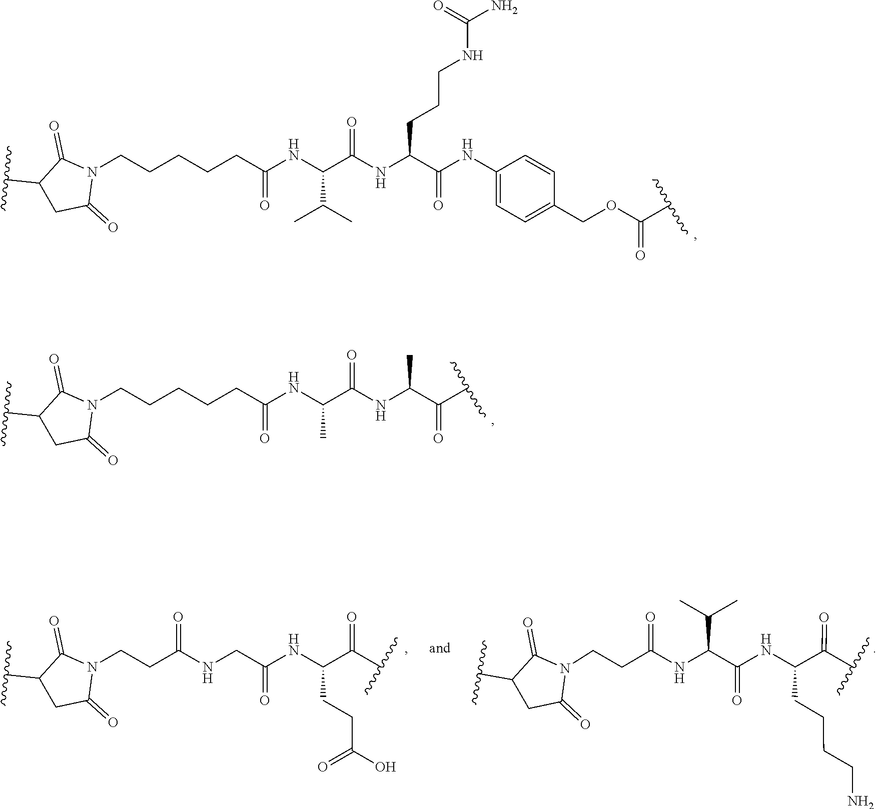 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)[C@H](C)NC(=O)[C@H](C)NC(=O)CCCCCN1C(=O)CC(*)C1=O ; *C(=O)[C@H](CCC(=O)O)NC(=O)CNC(=O)CCN1C(=O)CC(*)C1=O ; *C(=O)[C@H](CCCCN)NC(=O)[C@@H](NC(=O)CCN1C(=O)CC(*)C1=O)C(C)C]

In a particularly preferred embodiment, the compound of the present disclosure is selected from the following compounds, or tautomers, enantiomers or diastereoisomers thereof, or mixtures of isomers thereof, or pharmaceutically acceptable salts or solvates thereof:

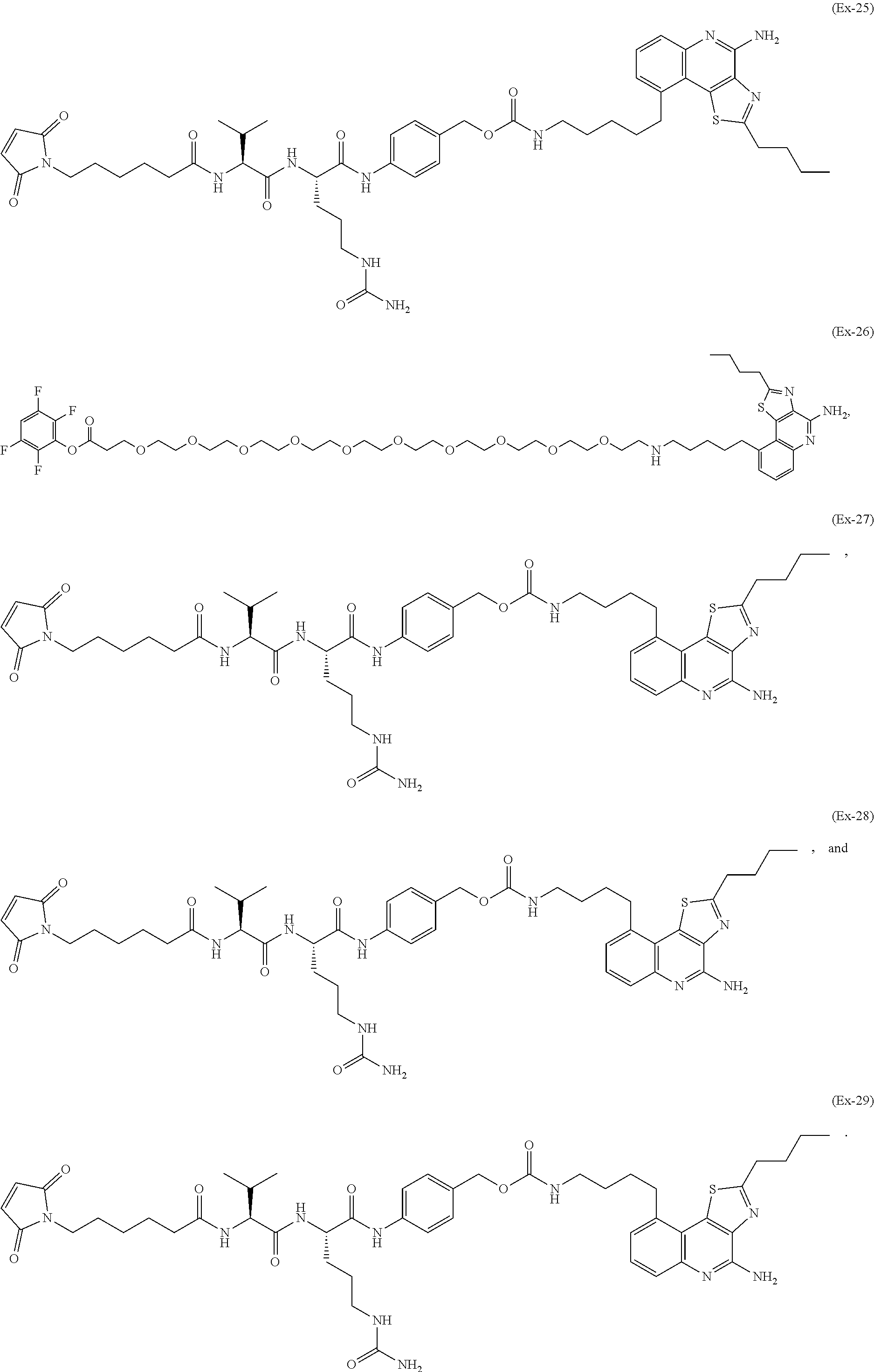

In certain preferred embodiments of the present disclosure, the present disclosure provides a ligand-drug conjugate of formula (B-2) below, or a tautomer, an enantiomer or a diastereoisomer thereof, or a mixture of isomers thereof, or a pharmaceutically acceptable salt or solvate thereof:

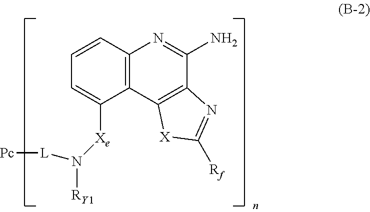

wherein X, Xₑ, R_{f}, and R_{Y1} are as defined above; L is the linker unit as defined above; n is an integer or a decimal from 1 to 10, preferably an integer or a decimal from 2 to 8; Pc is a ligand, preferably an antibody or an antigen-binding fragment thereof.

In a more preferred embodiment, Pc is an antibody or an antigen-binding fragment thereof that targets GPC3, Trop2, and HER2; preferably, the antibody is selected from: trastuzumab, pertuzumab, sacituzumab, and codrituzumab.

### Pharmaceutical Composition

In another aspect, the present disclosure provides a pharmaceutical composition, comprising the compound or the conjugate thereof, or the isomer thereof, or the mixture thereof, or the pharmaceutically acceptable salt thereof of the present application, and a pharmaceutically acceptable carrier or excipient, or comprising the ligand-drug conjugate, or the isomer thereof, or the mixture thereof, or the pharmaceutically acceptable salt thereof of the present application, and a pharmaceutically acceptable carrier or excipient.

The pharmaceutical composition described herein may comprise, in addition to the active compound, one or more adjuvants, which may be selected from the group consisting of the following ingredients: a filler (diluent), a binder, a wetting agent, a disintegrant, an excipient, and the like. Depending on the method of administration, the composition may comprise 0.1 wt. % to 99% wt. % of the active compound.

The pharmaceutical composition comprising the active ingredient may be in a form suitable for oral administration, such as tablet, troche, lozenge, aqueous or oil suspension, dispersible powder or granule, emulsion, hard or soft capsule, or syrup. Oral compositions may be prepared according to any method for preparing pharmaceutical compositions known in the art, and the compositions may comprise a binder, a filler, a lubricant, a disintegrant, a pharmaceutically acceptable wetting agent, and the like, and may further comprise one or more ingredients that are selected from the group consisting of: a sweetening agent, a flavoring agent, a colorant, and a preservative.

Aqueous suspensions may comprise the active substance in admixture with an excipient suitable for the formulation of aqueous suspensions. Aqueous suspensions may also comprise one or more preservatives, for example, one or more colorants, one or more flavoring agents, and one or more sweetening agents. Oil suspensions may be formulated by suspending the active ingredient in a vegetable oil. Oil suspensions may comprise a thickening agent. The sweetening agents and the flavoring agents described above may also be added.

The pharmaceutical compositions may also be prepared from a dispersible powder or granule that provides the active ingredient for preparing the aqueous suspension by adding water to mix the active ingredient with one or more of a dispersing agent, a wetting agent, a suspending agent, or a preservative. Other excipients, such as sweetening agents, flavoring agents and colorants, may also be added. These compositions are well preserved by the addition of antioxidants such as ascorbic acid. The pharmaceutical composition of the present application may also be in the form of an oil-in-water emulsion.

The pharmaceutical composition may be in the form of a sterile aqueous solution for injection. Available and acceptable vehicles or solvents include water, Ringer's solution, and isotonic sodium chloride solution. The sterile formulation for injection may be a sterile oil-in-water microemulsion for injection in which the active ingredient is dissolved in the oil phase. For example, the active ingredient is dissolved in a mixture of soybean oil and lecithin. The oil solution may then be added to a mixture of water and glycerol and treated to form a microemulsion. The injection or microemulsion can be locally injected into the blood of a patient in large quantities. Alternatively, it may be desirable to administer the solution and microemulsion in such a way that the compound of the present application is kept at a constant circulating concentration. To keep such a constant concentration, a continuous intravenous delivery device may be used. For example, the device may be a Deltec CADD-PLUS™. 5400 intravenous injection pump.

The pharmaceutical composition may be in the form of a sterile aqueous injection or oily suspension for intramuscular and subcutaneous administration. The suspension may be prepared according to existing techniques using suitable dispersing agents or wetting agents and suspending agents described above. The sterile formulation for injection may also be a sterile injection or suspension prepared in a parenterally acceptable non-toxic diluent or solvent. Alternatively, a sterile fixing oil may be conveniently used as a solvent or a suspending medium.

The compound of the present application may be administered in the form of a suppository for rectal administration. Such a pharmaceutical composition can be prepared by mixing a drug with a suitable non-irritative excipient, which is a solid at ambient temperature but a liquid in the rectum and therefore will melt in the rectum to release the drug. Such materials include cocoa butter, glycerinated gelatin, hydrogenated vegetable oils, and mixtures of polyethylene glycols of various molecular weights and fatty acid esters of polyethylene glycol.

As is well known to those skilled in the art, the dose of the drug administered depends on a variety of factors, including but not limited to, the activity of the particular compound employed, the age of the patient, the weight of the patient, the health condition of the patient, the behavior of the patient, the diet of the patient, the time of administration, the mode of administration, the rate of excretion, the combination of drugs, and the like. In addition, the optimal treatment regimen, such as the mode of treatment, the compound or the tautomer, the mesomer, the racemate, the enantiomer or the diastereoisomer thereof, or the mixture thereof, or the pharmaceutically acceptable salt thereof described herein, and/or the daily amount of the compound or the tautomer, the mesomer, the racemate, the enantiomer or the diastereoisomer thereof, or the mixture thereof, or the pharmaceutically acceptable salt thereof, or the identity of the pharmaceutically acceptable salt, can be verified according to conventional treatment regimens.

Prevention and/or Treatment of Tumors

In another aspect, the present application provides a method for influencing Toll-like receptor (TLR) functionality, comprising administering the compound or the conjugate thereof, or the isomer thereof, or the mixture thereof, or the pharmaceutically acceptable salt thereof of the present application, or administering the ligand-drug conjugate, or the isomer thereof, or the mixture thereof, or the pharmaceutically acceptable salt thereof of the present application, or administering the pharmaceutical composition of the present application.

In another aspect, the present application provides a method for modulating immune system functionality, comprising administering the compound or the conjugate thereof, or the isomer thereof, or the mixture thereof, or the pharmaceutically acceptable salt thereof of the present application, or administering the ligand-drug conjugate, or the isomer thereof, or the mixture thereof, or the pharmaceutically acceptable salt thereof of the present application, or administering the pharmaceutical composition of the present application.

In another aspect, the present application provides use of the compound or the conjugate thereof, or the isomer thereof, or the mixture thereof, or the pharmaceutically acceptable salt thereof of the present application, or the ligand-drug conjugate, or the isomer thereof, or the mixture thereof, or the pharmaceutically acceptable salt thereof of the present application, or the pharmaceutical composition of the present application in the preparation of a medicament for preventing and/or treating a disease and/or a symptom.

In some embodiments, in the use or the method described herein, the disease and/or the symptom includes a disease and/or a symptom associated with Toll-like receptor (TLR) signaling.

In some embodiments, in the use or the method described herein, the disease and/or the symptom is selected from the group consisting of: a tumor, an autoimmune disease, an inflammation, sepsis, an allergy, asthma, transplant rejection, graft-versus-host disease, immunodeficiency, and an infection caused by a virus.

In some embodiments, in the use or the method described herein, the disease and/or the symptom is a tumor selected from the group consisting of tumors associated with expression of the following: GPC3, Trop2, and HER2. In some embodiments, the disease and/or the symptom is a tumor selected from: melanoma, lung cancer, liver cancer, basal cell carcinoma, kidney cancer, myeloma, biliary tract cancer, brain cancer, breast cancer, cervical cancer, choriocarcinoma, colon cancer, rectal cancer, head and neck cancer, peritoneal tumor, fallopian tube cancer, endometrial cancer, esophageal cancer, gastric cancer, leukemia, lymphoma, sarcoma, neuroblastoma, oral cancer, ovarian cancer, pancreatic cancer, prostate cancer, testicular cancer, skin cancer, and thyroid cancer.

In some embodiments, in the use or the method described herein, the disease and/or the symptom is an infection caused by a virus selected from the group consisting of: dengue virus, yellow fever virus, West Nile virus, Japanese encephalitis virus, tick-borne encephalitis virus, Kunjin virus, Murray Valley encephalitis virus, St. Louis encephalitis virus, Omsk hemorrhagic fever virus, bovine viral diarrhea virus, Zika virus, HIV (human immunodeficiency virus), HBV (hepatitis B virus), HCV (hepatitis C virus), HPV (human papillomavirus), RSV (respiratory syncytial virus), SARS-CoV (severe acute respiratory syndrome coronavirus), SARS-CoV-2 (severe acute respiratory syndrome coronavirus 2), MERS-CoV (middle east respiratory syndrome coronavirus), and influenza virus.

The compound described herein may influence the activity of TLR. The influence on the activity may be a 1% or greater, 2% or greater, 4% or greater, 5% or greater, 8% or greater, 10% or greater, 15% or greater, 18% or greater, 20% or greater, 25% or greater, 40% or greater, 50% or greater, 60% or greater, 70% or greater, 80% or greater, 90% or greater, or 95% or greater increase in TLR activity when the compound of the present application is added to a culture medium, as compared with the addition of a negative control or a reference drug. For example, the influence on the activity may be an EC₅₀ value (nM) of 10000 or less, 5000 or less, 4000 or less, 3000 or less, 2000 or less, 1000 or less, 500 or less, 400 or less, 300 or less, 200 or less, 150 or less, 120 or less, 110 or less, 100 or less, 99 or less, 98 or less, 97 or less, 95 or less, 90 or less, 80 or less, 75 or less, 70 or less, 65 or less, 62 or less, 60 or less, 50 or less, 40 or less, 30 or less, 25 or less, 23 or less, 22 or less, 20 or less, 19 or less, 18 or less, 18.5 or less, 17 or less, 15 or less, 12 or less, 10 or less, 9 or less, 8.5 or less, 7 or less, 6.7 or less, 6 or less, 5.9 or less, 5.5 or less, 5.0 or less, 4.8 or less, 4.5 or less, 4.4 or less, 4 or less, 3.5 or less, 3 or less, 2.5 or less, 2 or less, 1.5 or less, 1.0 or less, 0.5 or less, 0.3 or less, 0.29 or less, 0.25 or less, 0.21 or less, 0.20 or less, 0.18 or less, 0.17 or less, 0.15 or less, 0.12 or less, 0.10 or less, 0.09 or less, 0.08 or less, 0.07 or less, 0.06 or less, 0.05 or less, 0.04 or less, 0.03 or less, 0.02 or less, or 0.01 or less for TLR. For example, the immune cell may include, but is not limited to, granulocytes and/or agranulocytes. For example, the immune cell includes, but is not limited to, B cells, T cells, natural killer cells, monocytes, macrophages, mast cells, and/or dendritic cells. For example, the immune cell includes PBMCs. For example, TLR includes human TLR. For example, TLR includes TLR7 and/or TLR8.

The compounds described herein may influence the ability of immune cells to express and/or release cytokines. The influence on the activity may be a 1% or greater, 2% or greater, 4% or greater, 5% or greater, 8% or greater, 10% or greater, 15% or greater, 18% or greater, 20% or greater, 25% or greater, 40% or greater, 50% or greater, 60% or greater, 70% or greater, 80% or greater, 90% or greater, or 95% or greater increase in the ability of immune cells to express and/or release cytokines when the compound of the present application is added to a culture medium containing immune cells or administered to a subject, as compared with the addition or administration of a negative control or a reference drug. For example, the immune cell may include, but is not limited to, granulocytes and/or agranulocytes. For example, the immune cell includes, but is not limited to, B cells, T cells, natural killer cells, monocytes, macrophages, mast cells, and/or dendritic cells. For example, the immune cell includes PBMCs. For example, the cytokine may be an immune cell cytokine. For example, the cytokine may be TNF-α and/or IFN-α

The compounds described herein may influence the activity of immune cells. The influence on the activity may be a 1% or greater, 2% or greater, 4% or greater, 5% or greater, 8% or greater, 10% or greater, 15% or greater, 18% or greater, 20% or greater, 25% or greater, 40% or greater, 50% or greater, 60% or greater, 70% or greater, 80% or greater, 90% or greater, or 95% or greater increase in immune cell activity when the compound of the present application is added to a culture medium containing immune cells, as compared with the addition of a negative control or a reference drug. For example, the influence on the activity may be an EC₅₀ value (nM) of 10000 or less, 5000 or less, 4000 or less, 3000 or less, 2000 or less, 1000 or less, 500 or less, 400 or less, 300 or less, 200 or less, 150 or less, 120 or less, 110 or less, 100 or less, 99 or less, 98 or less, 97 or less, 95 or less, 90 or less, 80 or less, 75 or less, 70 or less, 65 or less, 62 or less, 60 or less, 50 or less, 40 or less, 30 or less, 25 or less, 23 or less, 22 or less, 20 or less, 19 or less, 18 or less, 18.5 or less, 17 or less, 15 or less, 12 or less, 10 or less, 9 or less, 8.5 or less, 7 or less, 6.7 or less, 6 or less, 5.9 or less, 5.5 or less, 5.0 or less, 4.8 or less, 4.5 or less, 4.4 or less, 4 or less, 3.5 or less, 3 or less, 2.5 or less, 2 or less, 1.5 or less, 1.0 or less, 0.5 or less, 0.3 or less, 0.29 or less, 0.25 or less, 0.21 or less, 0.20 or less, 0.18 or less, 0.17 or less, 0.15 or less, 0.12 or less, 0.10 or less, 0.09 or less, 0.08 or less, 0.07 or less, 0.06 or less, 0.05 or less, 0.04 or less, 0.03 or less, 0.02 or less, or 0.01 or less for immune cells. For example, the immune cell may include, but is not limited to, granulocytes and/or agranulocytes. For example, the immune cell includes, but is not limited to, B cells, T cells, natural killer cells, monocytes, macrophages, mast cells, and/or dendritic cells. For example, the immune cell includes PBMCs.

### BRIEF DESCRIPTION OF THE DRAWINGS

FIG. 1: (a) TNF-α release from human whole blood in the presence of different concentrations of antibody-drug conjugates; (b) IFN-α release from human whole blood in the presence of different concentrations of antibody-drug conjugates.

FIG. 2: (a) results of a co-incubation experiment with the control ADC1, tumor cells, and human peripheral blood mononuclear cells; (b) results of a co-incubation experiment with ADC6, tumor cells, and human peripheral blood mononuclear cells.

FIG. 3: IFN-α release from a co-incubation experiment with ADC-13 and HepG2 & PBMCs.

FIG. 4: IL-6 release from the co-incubation experiment with ADC-13 and HepG2 & PBMCs.

FIG. 5: TNF-α release from the co-incubation experiment with ADC-13 and HepG2 & PBMCs.

FIG. 6: experimental results of in vivo efficacy experiment 1 in an hGPC3-MC38 colon cancer animal model.

FIG. 7: experimental results of in vivo efficacy experiment 2 in an hGPC3-MC38 colon cancer animal model.

FIG. 8: results of a rechallenge experiment of tumor cells in an MC38/hTLR8 KI mouse model that does not express hGPC3.

### DEFINITIONS OF TERMS

In the present application, the terms “Toll-like receptor” and “TLR” generally refer to any member of the highly conserved mammal pattern recognition receptor family that recognizes pathogen-associated molecular patterns (PAMPs) and serves as a key signaling element in innate immunity. TLR polypeptides share a characteristic structure, comprising an extracellular domain with leucine-rich repeats, a transmembrane domain, and an intracellular domain involved in TLR signaling. TLR includes, but is not limited to, human TLR.

In the present application, the terms “Toll-like receptor 7” and “TLR7” refer to a nucleic acid or polypeptide having at least 70%, 80%, 90%, 95%, 96%, 97%, 98%, 99%, or higher sequence identity with published TLR7 sequences, such as human TLR7 polypeptide of GenBank Accession No. AAZ99026, or mouse TLR7 polypeptide of GenBank Accession No. AAK62676.

In the present application, the terms “Toll-like receptor 8” and “TLR8” refer to a nucleic acid or polypeptide having at least 70%, 80%, 90%, 95%, 96%, 97%, 98%, 99%, or higher sequence identity with published TLR8 sequences, such as human TLR8 polypeptide of GenBank Accession No. AAZ95441, or mouse TLR8 polypeptide of GenBank Accession No. AAK62677.

In the present application, the term “TLR agonist” generally refers to an agent that, either directly or indirectly, binds to TLR (e.g., TLR7 and/or TLR8) to induce TLR signaling. Any detectable difference in TLR signaling may indicate that the agonist stimulates or activates TLR. Differences in signaling may be represented by changes in target gene expression, changes in the phosphorylation of signaling components, changes in the intracellular localization of downstream elements such as nuclear factor-κB(NF-κB), changes in the association of certain components such as IL-1 receptor-associated kinase (IRAK) with other proteins or intracellular structures, or changes in the biochemical activity of components such as kinases (e.g., mitogen-activated protein kinase (MAPK)). In the present application, the term “halogen” generally refers to fluorine, chlorine, bromine, or iodine, and it may be, for example, fluorine or chlorine.

In the present application, the term “alkyl” generally refers to a residue derived from an alkane by the removal of a hydrogen atom. The alkyl may be substituted or unsubstituted, or replaced or unreplaced. The term “alkyl” generally refers to a saturated linear or branched aliphatic hydrocarbon group having a residue derived from a parent alkane by the removal of hydrogen atoms from the same carbon atom or two different carbon atoms, and it may be a linear or branched group containing 1 to 20 carbon atoms, e.g., 1 to 12 carbon atoms, such as alkyl containing 1 to 6 carbon atoms. Non-limiting examples of alkyl include but are not limited to methyl, ethyl, propyl, propyl, butyl, etc. The alkyl may be substituted or unsubstituted, or replaced or unreplaced. For example, when substituted, the alkyl may be substituted at any available connection site with a substituent that may be independently optionally selected from one or more of alkyl, alkenyl, alkynyl, alkoxy, alkylthio, alkylamino, halogen, sulfhydryl, hydroxy, nitro, cyano, cycloalkyl, heterocyclyl, aryl, heteroaryl, cycloalkoxy, heterocycloalkoxy, cycloalkylthio, heterocycloalkylthio, and oxo, and the substituent may, e.g., be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H, or a C₁₋₆ aliphatic group.

In the present application, the term “alkoxy” generally refers to the aforementioned alkyl having a specified number of carbon atoms connected through an oxygen bridge, unless otherwise specified; C₁₋₆ alkoxy includes C₁, C₂, C₃, C₄, C₅ and C₆ alkoxy groups. Examples of alkoxy include, but are not limited to: methoxy, ethoxy, n-propoxy, isopropoxy, n-butoxy, sec-butoxy, tert-butoxy, n-pentoxy, and S-pentoxy.

In the present application, the term “alkylene” generally refers to a saturated linear or branched aliphatic hydrocarbon group having 2 residues derived from a parent alkane by the removal of two hydrogen atoms from the same carbon atom or two different carbon atoms, and it may be a linear or branched group containing 1 to 20 carbon atoms; for example, the term “methylene” may refer to a residue derived from a one-carbon atom group by removal of two hydrogen atoms. Methylene may be substituted or unsubstituted, or replaced or unreplaced; for example, the alkylene contains 1 to 12 carbon atoms, e.g., alkylene containing 1 to 6 carbon atoms. Non-limiting examples of alkylene include but are not limited to methylene (—CH₂—), 1,1-ethylene (—CH(CH₃)—), 1,2-ethylene (—CH₂CH₂—), 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), 1,5-butylene (—CH₂CH₂CH₂CH₂CH₂—), etc. The alkylene may be substituted or unsubstituted, or replaced or unreplaced. For example, when substituted, the alkylene may be substituted at any available connection site with a substituent that may be independently optionally selected from one or more of alkyl, alkenyl, alkynyl, alkoxy, alkylthio, alkylamino, halogen, sulfhydryl, hydroxy, nitro, cyano, cycloalkyl, heterocyclyl, aryl, heteroaryl, cycloalkoxy, heterocycloalkoxy, cycloalkylthio, heterocycloalkylthio, and oxo, and, for example, may be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H, or C₁₋₆ aliphatic group. Methylene or the alkylene may be substituted or unsubstituted. In the present application, the term “alkenyl” generally refers to a linear or branched hydrocarbon group containing one or more double bonds. Exemplary examples of alkenyl include allyl, homoallyl, vinyl, crotyl, butenyl, pentenyl, hexenyl, etc. Exemplary instances of C2-6 alkenyl containing one or more double bonds include butadienyl, pentadienyl, hexadienyl, and hexatrienyl, as well as branched forms thereof. The positions of the unsaturated bonds (double bonds) may be any positions in the carbon chain. The alkenyl may be substituted or unsubstituted.

In the present application, the term “alkenylene” generally refers to a residue derived from an alkene by the removal of two hydrogen atoms from a carbon atom. For example, the alkenylene may be acrol, vinylene, butenylene, pentenylene, hexenylene, etc. The alkenylene may be substituted or unsubstituted.

In the present application, the term “alkynyl” generally refers to unsaturated linear or branched alkynyl, e.g., ethynyl, 1-propynyl, propargyl, butynyl, etc. The alkynyl may be substituted or unsubstituted.

In the present application, the term “alkynylene” generally refers to a residue derived from an alkyne by the removal of two hydrogen atoms from a carbon atom. For example, the alkynylene may be ethynylene, propynylene, propargylene, butynylene, etc. The alkynylene may be substituted or unsubstituted.

In the present application, the term “aryl” generally refers to a residue derived from an aromatic ring by the removal of a hydrogen atom. The term “aromatic ring” may refer to a 6- to 14-membered all-carbon monocyclic ring or fused polycyclic ring (i.e., rings that share adjacent pairs of carbon atoms) having a conjugated 7r-electron system, and it may be 6- to 10-membered, such as benzene and naphthalene. The aromatic ring can be fused to a heteroaryl, heterocyclyl or cycloalkyl ring, wherein the ring connected to the parent structure is the aryl ring. The aryl may be substituted or unsubstituted, and when it is substituted, the substituent may be one or more of the following groups independently selected from the group consisting of: alkyl, alkenyl, alkynyl, alkoxy, alkylthio, alkylamino, halogen, sulfhydryl, hydroxy, nitro, cyano, cycloalkyl, heterocycloalkyl, aryl, heteroaryl, cycloalkoxy, heterocycloalkoxy, cycloalkylthio, and heterocycloalkylthio. The aryl may be substituted or unsubstituted.

In the present application, the term “arylene” generally refers to a residue derived from an aromatic ring by the removal of two hydrogen atoms from carbon atoms. For example, the arylene may be phenylene and naphthylene. The arylene may be substituted or unsubstituted.

In the present application, the term “heteroaryl” generally refers to a residue derived from a heteroaromatic ring by removal of a hydrogen atom from a carbon atom. The term “heteroaromatic ring” refers to a heteroaromatic system comprising 1 to 4 heteroatoms and 5 to 14 ring atoms, wherein the heteroatoms may be selected from the group consisting of: oxygen, sulfur and nitrogen. Heteroaryl may be 5- to 10-membered and may be 5- or 6-membered, such as furanyl, thienyl, pyridinyl, pyrrolyl, N-alkylpyrrolyl, pyrimidinyl, pyrazinyl, imidazolyl, and tetrazolyl, etc. The heteroaryl may be fused to an aryl, heterocyclyl or cycloalkyl ring, wherein the ring connected to the parent structure is the heteroaryl ring. Heteroaryl may be optionally substituted or unsubstituted, and when it is substituted, the substituent may be one or more of the following groups independently selected from the group consisting of: alkyl, alkenyl, alkynyl, alkoxy, alkylthio, alkylamino, halogen, sulfhydryl, hydroxy, nitro, cyano, cycloalkyl, heterocycloalkyl, aryl, heteroaryl, cycloalkoxy, heterocycloalkoxy, cycloalkylthio and heterocycloalkylthio. The heteroaryl may be substituted or unsubstituted.

In the present application, the term “heteroarylene” generally refers to a residue derived from a heteroaromatic ring by the removal of two hydrogen atoms from carbon atoms. For example, the heteroarylene may be furanylene, thienylene, pyridinylene, pyrrolylene, pyrimidinylene, pyrazinylene, imidazolylene, tetrazolylene, etc. The heteroarylene may be substituted or unsubstituted.

In the present application, the term “alcyl” generally refers to a residue derived from an aliphatic ring by the removal of a hydrogen atom from the same carbon atom or from a plurality of different carbon atoms. The term “cycloalkane” generally refers to a saturated or partially unsaturated monocyclic or polycyclic cyclic hydrocarbon, and the carbon ring contains 3 to 20 carbon atoms, may contain 3 to 12 carbon atoms, may contain 3 to 10 carbon atoms, and may contain 3 to 8 carbon atoms. Non-limiting examples of alcyl include cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic carbon rings may include spiro, fused, and bridged carbon rings. The alcyl may be substituted or unsubstituted. In the present application, the term “carbocyclyl” generally refers to a residue derived from a carbon ring by the removal of a hydrogen atom from a carbon atom. The term “carbon ring” generally refers to a saturated or partially unsaturated monocyclic or polycyclic hydrocarbon, which contains 3 to 20 carbon atoms, may contain 3 to 12 carbon atoms, may contain 3 to 10 carbon atoms, and may contain 3 to 8 carbon atoms. Non-limiting examples of a monocyclic carbon ring include cyclopropane, cyclobutane, cyclopentane, cyclopentene, cyclohexane, cyclohexene, cyclohexadiene, cycloheptane, cycloheptatriene, cyclooctane, etc.; polycyclic carbon rings may include spiro, fused and bridged carbon rings. The carbocyclyl may be substituted or unsubstituted. In some instances, alicyclic and carbocyclic rings are used interchangeably.

In the present application, the term “partially unsaturated” generally means that the cyclic structure contains at least one double or triple bond between the ring molecules. The term “partially unsaturated” encompasses cyclic structures having multiple sites of unsaturation, but is not intended to include aromatic or heteroaromatic rings defined herein. The term “unsaturated” means that the moiety has one or more degrees of unsaturation.

In the present application, the term “alcylene” generally refers to a residue derived from an alicyclic ring by the removal of two hydrogen atoms from carbon atoms. For example, the alcylene may be cyclopropylene, cyclobutylene, cyclopentylene, cyclopentenylene, cyclohexylene, cyclohexenylene, cyclohexadienylene, cycloheptylene, cycloheptatrienylene, cyclooctylene, etc.; polycyclic carbon rings may include spiro, fused and bridged carbon rings. The alcylene may be substituted or unsubstituted.

In the present application, the term “aliphatic heterocyclyl” generally refers to a 3- to 7-membered monocyclic carbon ring structure, a fused 7- to 10-membered bicyclic heterocyclic structure, or a bridged 6- to 10-membered bicyclic heterocyclic structure that is stable and non-aromatic. Such cyclic structures may be saturated or partially saturated, and also contain one or more heteroatoms in addition to carbon atoms, wherein the heteroatoms may be selected from the group consisting of: oxygen, sulfur, and nitrogen. For example, they contain 1 to 4 heteroatoms as defined above. When used to refer to atoms on an aliphatic heterocyclic structure, the term “nitrogen” may include nitrogen that has undergone a substitution reaction. For example, the aliphatic heterocyclyl may comprise “heterocycloalkyl” that may refer to a 3- to 7-membered monocyclic alkyl structure, a fused 7- to 10-membered heterobicyclic structure, or a bridged 6- to 10-membered heterobicyclic structure that is stable and non-aromatic. Such cyclic structures further contain one or more heteroatoms in addition to carbon atoms, wherein the heteroatoms may be selected from the group consisting of: oxygen, sulfur, and nitrogen. For example, they contain 1 to 4 heteroatoms as defined above. The heterocycloalkyl may be substituted or unsubstituted. The aliphatic heterocyclyl may be substituted or unsubstituted.

In the present application, the term “aliphatic heterocyclylene” generally refers to a residue derived from an alicyclic ring by the removal of two hydrogen atoms from carbon atoms. The aliphatic heterocyclylene may be substituted or unsubstituted.

In the present application, the term “ring-forming atom” generally refers to an atom contained in a cyclic structure. For example, a ring-forming atom may be a carbon atom in a benzene ring or may be a nitrogen atom in a pyridine ring. When a hydrogen atom is connected to a ring-forming atom, the ring-forming atom may be substituted or unsubstituted.

In the present application, the term “each independently” or “each . . . is independently” generally means that a variable applies in any case irrespective of the presence or absence of variables having the same or different definitions in the same compound. For example, the variable may refer to the type or number of substituents in the compound, the type of atoms in the compound, and so on. For example, where R occurs twice in a compound and R is defined as “independently carbon or nitrogen”, both R may be carbon, both R may be nitrogen, or one R may be carbon while the other R is nitrogen.

In the present application, the term “optional” or “optionally” generally means that the event or circumstance subsequently described may, but not necessarily occur, and that the description includes instances where the event or circumstance occurs or does not occur. For example, “heterocyclyl group optionally substituted with alkyl” means that alkyl may be, but not necessarily present, and that the description may include instances where the heterocyclyl group is or is not substituted with alkyl.

In the present application, the term “substituted” generally means that one or more hydrogen atoms in the group, for example, up to 5 (e.g., 1-3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the term “0 or more (e.g., 0 or 1 or more, 0 or 1, or 0) methylene units are replaced” generally means that when the structure comprises 1 or more methylene units, the one or more methylene units may be unreplaced, or may be replaced by one or more groups that are not methylene (e.g., —NHC(O)—, —C(O)NH—, —C(O)—, —OC(O)—, —C(O)O—, —NH—, —O—, —S—, —SO—, —SO₂—, —PH—, —P(═O)H—, —NHSO₂—, —SO₂NH—, —C(═S)—, —C(═NH)—, —N═N—, —C═N—, —N═C—, or —C(=N₂)—).

In the present application, the “linking” of group X to group Y may generally be in any orientation, which generally means that when group X is used for linker Y and group Z, two or more linking sites of group X may be linked arbitrarily to either group Y or group Z.

In the present application, the term “compound” generally refers to a substance having two or more different elements. For example, the compound of the present application may be an organic compound. For example, the compound described herein may be a compound having a molecular weight of 500 or less, a compound having a molecular weight of 1000 or less, a compound having a molecular weight of 1000 or greater, or a compound having a molecular weight of 10,000 or greater, or 100,000 or greater. In the present application, the compound may further refer to a compound that involves linking by a chemical bond, for example, a compound where one or more molecules having a molecular weight of 1000 or less are linked, via a chemical bond, to a biological macromolecule, wherein the biological macromolecule may be polysaccharide, protein, nucleic acid, polypeptide, and the like. For example, the compound of the present application may be a compound comprising a protein and one or more molecules having a molecular weight of 1000 or less linked to the protein, a compound comprising a protein and one or more molecules having a molecular weight of 10,000 or less linked to the protein, or a compound comprising a protein and one or more molecules having a molecular weight of 100,000 or less linked to the protein.

In the present application, the terms such as “alkyl”, “alkenyl” and “cycloalkyl” may be preceded by a notation to indicate the number of atoms present in the group under particular circumstances as in C₁-C₄ alkyl, C₃-C₇ cycloalkoxy, and C₁-C₄ alkylcarbonylamino and the like, as known to those skilled in the art, and the subscript numeral following “C” indicates the number of carbon atoms present in the group. For example, C₃ alkyl refers to an alkyl group having three carbon atoms (e.g., n-propyl or isopropyl); in C₁₋₁₀, members of the group may have any number of carbon atoms within the range of 1-10.

One or more hydrogen atoms in the group, for example, up to 5 (e.g., 1-3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the compound of the present application includes tautomers, mesomers, racemates, enantiomers, and/or diastereoisomers thereof. In the present application, the term “diastereoisomer” generally refers to a stereoisomer that has two or more chiral centers and whose molecules are not mirror images of each other. Diastereoisomers may have different physical properties, e.g., melting points, boiling points, spectral properties, and reactivities. In the present application, the terms “tautomer” and “tautomeric form” are used interchangeably and generally refer to structural isomers of different energies that can be converted into each other by crossing a low energy barrier. For example, proton tautomers (also known as prototropic tautomers) include interconversions by proton migration, such as keto-enol isomerization and imine-enamine isomerization. Valence tautomers include interconversions by recombination of some bonding electrons. In the present application, the term “mesomer” generally means that the molecule contains asymmetric atoms but the total optical rotation is zero due to the presence of symmetric factors. The term “racemate” or “racemic mixture” refers to a composition of two enantiomeric substances in equimolar amounts.

In the present application, certain atoms of the compound of the present application may be present in one or more isotopic forms. For example, hydrogen may occur as protium (¹H), deuterium (²H), and tritium (³H), and carbon may naturally occur as three different isotopes (¹²C, ¹³C, and ¹⁴C). Examples of isotopes that can be incorporated into the compound of the present application also include but are not limited to ¹⁵N, ¹⁸O, ¹⁷O, ¹⁸F, ³²P, ³³P, ¹²⁹I, ¹³¹I, ¹²³I, ¹²⁴I, ¹²⁵I, or similar isotopes.

Thus, one or more of such isotopes may be enriched in the compounds of the present application relative to the natural abundance of such isotopes. Such isotopically enriched compounds can be used for a variety of purposes, as known to those skilled in the art. For example, replacement with heavy isotopes such as deuterium (²H) may offer certain therapeutic advantages, possibly due to higher metabolic stability. For example, the natural abundance of deuterium (²H) is about 0.015%. Accordingly, one out of about 6500 hydrogen atoms is a deuterium atom. Accordingly, the deuterium abundance of one or more sites (as the case may be) in a deuterium-containing compound of the present application is greater than 0.015%. Unless otherwise indicated, the structures described herein may also include compounds that differ only in the presence or absence of one or more isotopically enriched atoms. For example, compounds having a structure identical to the structure disclosed herein except for the substitution of the hydrogen atom by deuterium or tritium or the substitution of the carbon atom by carbon 13 or carbon 14 shall fall within the scope of the present application.

In the present application, the term “isomers” of a compound or a ligand-drug conjugate generally includes tautomers, mesomers, racemates, stereoisomers, enantiomers or diastereoisomers thereof, or mixtures thereof.

In the present application, the term “ligand-drug conjugate” generally means that a ligand is linked to a biologically active cytotoxic drug via a stable linking unit. In the present application, the “ligand-drug conjugate” may be an antibody-drug conjugate (ADC), which may mean that a monoclonal antibody or an antibody fragment is linked to a biologically active cytotoxic drug via a stable linking unit.

In the present application, the term “ligand” generally refers to a macromolecular compound capable of recognizing and binding to an antigen or receptor associated with a target cell. The role of ligands may be to present the drug to a target cell population to which the ligand binds, and the ligands include, but are not limited to, protein hormones, lectin, growth factors, antibodies, or other molecules capable of binding to a cell, a receptor and/or an antigen. In the present application, the ligand may be represented as Pc; the ligand antigen forms a linking bond with the linking unit through a heteroatom on the ligand, and the ligand may be an antibody or an antigen-binding fragment thereof (Ab), wherein the antibody may be selected from a chimeric antibody, a humanized antibody, a fully human antibody, or a murine antibody, and the antibody may be a monoclonal antibody. For example, the antibody may be an antibody that targets the following target points: HER2, TROP2, or GPC3. For example, the antibody may be an antibody that targets the following target points: 5T4, AGS-16, ANGPTL4, ApoE, CD19, CTGF, CXCR5, FGF2, MCPT8, MFI2, MS4A7, NCA, Sema5b, SLITRK6, STC2, TGF, 0772P, 5T4, ACTA2, ADGRE1, AG-7, AIF1, AKR1C₁, AKR1C₂, ASLG659, Axl, B7H3, BAFF-R, BCMA, BMPR1B, BNIP3, C1QA, C1QB, CA6, CADM1, CCD79b, CCL5, CCR5, CCR7, CD11c, CD123, CD138, CD142, CD147, CD166, CD19, CD19, CD22, CD21, CD20, CD205, CD22, CD223, CD228, CD25, CD30, CD33, CD37, CD38, CD40, CD45, CD45 (PTPRC), CD46, CD47, CD49D (ITGA4), CD56, CD66e, CD70, CD71, CD72, CD74, CD79a, CD79b, CD80, CDCP1, CDH11, CD11b, CEA, CEACAM5, c-Met, COL6A3, COL7A1, CRIPTO, CSF1R, CTSD, CTSS, CXCL11, CXCL10, DDIT4, DLL3, DLL4, DR5, E16, EFNA4, EGFR, EGFRvIII, EGLN, EGLN3, EMR2, ENPP3, EpCAM, EphA2, EphB2R, ETBR, FcRH2, FcRHi, FGFR2, FGFR3, FLT3, FOLR-α, GD2, GEDA, GPC-1, GPNMB, GPR20, GZMB, HER2, HER3, HLA-DOB, HMOX1, IFI6, IFNG, IGF-1R, IGFBP3, IL10RA1, IL-13R, IL-2, IL20Ra, IL-3, IL-4, IL-6, IRTA2, KISSIR, KRT33A, LIV-1, LOX, LRP-1, LRRC15, LUM, LY64, LY6E, Ly86, LYPD3, MDP, MMP10, MMP14, MMP16, MPF, MSG783, MSLN, MUC-1, NaPi2b, Napi3b, Nectin-4, Nectin-4, NOG, P2X5, pCAD, P-Cadherin, PDGFRA, PDK1, PD-L1, PFKFB3, PGF, PGK1, PIK3AP1, PIK3CD, PLOD2, PSCA, PSCAhlg, PSMA, PSMA, PTK7, P-Cadherin, RNF43, NaPi2b, ROR1, ROR2, SERPINE1, SLC39A6, SLTRK6, STAT1, STEAP1, STEAP2, TCF4, TENB2, TGFB1, TGFB2, TGFBR1, TNFRSF21, TNFSF9, Trop-2, TrpM4, Tyro7, UPK1B, VEGFA, WNT5A, epidermal growth factors, brevican, mesothelin, sodium phosphate cotransporter 2B, Claudin 18.2, endothelin receptors, mucins (e.g., mucin 1 and mucin 16), guanylate cyclase C, integrin a4p7, integrin a5p6, trophoblast glycoprotein, or tissue factors.

In the present application, the term “cytotoxic drug” generally refers to a toxic drug, and the cytotoxic drug may be a chemical molecule within the tumor cell that is strong enough to disrupt its normal growth. Cytotoxic drugs can kill tumor cells at a sufficiently high concentration. The “cytotoxic drug” may include toxins, such as small molecule toxins or enzymatically active toxins of bacterial, fungal, plant or animal origin, radioisotopes (e.g., At²¹¹, I¹³¹, I¹²⁵, Y⁹⁰, Re¹⁸⁶, Re¹⁸⁸, Sm¹⁵³, Bi²¹², P³², or radioactive isotopes of Lu), toxic drugs, chemotherapeutic drugs, antibiotics, and nucleolytic enzymes; for example, the cytotoxic drug may be a toxic drug, including but not limited to a camptothecin derivative, which, for example, may be the camptothecin derivative exatecan (chemical name: (1S,9S)-1-amino-9-ethyl-5-fluoro-2,3-dihydro-9-hydroxy-4-methyl-1H,12H-benzo[de]pyrano[3′,4′:6,7]imidazo[1,2-b]quinoline-10,13 (9H,15H)-dione).

In the present application, the term “linker unit” or “linker structure” generally refers to a chemical structural fragment or bond that is linked to a ligand at one end and to a cytotoxic drug at the other end, or that is linked to other linkers before being linked to the cytotoxic drug. The direct or indirect linking of a ligand may mean that the group is directly linked to the ligand via a covalent bond, and may also be linked to the ligand via a linker structure. For example, the linker structure may be a structure represented by -Lₐₓ-L_{b}-L_{c}- and/or -Lₐ-L_{b}-L_{c}- described herein. For example, a chemical structure fragment or bond comprising an acid-labile linker structure (e.g., hydrazone), a protease-sensitive (e.g., peptidase-sensitive) linker structure, a photolabile linker structure, a dimethyl linker structure or a disulfide-containing linker structure may be used as a linker structure.

In the present application, the term a structure being “optionally linked to other molecular moieties” generally means that the structure is not linked to any other chemical structure, or that the structure is linked (e.g., via a chemical bond or a linker structure) to one or more other chemical structures (e.g., ligands described herein) different from the structure.

In the present application, the term “antibody or antigen-binding fragment thereof” generally refers to immunological binding reagents extending to all antibodies from all species, including dimeric, trimeric and multimeric antibodies, bispecific antibodies, chimeric antibodies, fully humanized antibodies, humanized antibodies, recombinant and engineered antibodies, and fragments thereof. The term “antibody or fragment thereof” may refer to any antibody-like molecule having an antigen-binding region, and includes small molecule fragments, such as Fab′, Fab, F(ab′)₂, single domain antibodies (DABs), Fv, scFv (single-chain Fv), linear antibodies, bispecific antibodies, and the like. The term “antigen-binding fragment” can refer to one or more fragments of an antibody that retain the ability to specifically bind to an antigen. For example, a fragment of a full-length antibody can be used to perform the antigen-binding function of the antibody. Techniques for preparing and using various antibody-based constructs and fragments are well known in the art. The antibody may include one or more of an anti-HER2 (ErbB2) antibody, an anti-EGFR antibody, an anti-B7-H3 antibody, an anti-c-Met antibody, an anti-HER3 (ErbB3) antibody, an anti-HER4 (ErbB4) antibody, an anti-CD20 antibody, an anti-CD22 antibody, an anti-CD30 antibody, an anti-CD33 antibody, an anti-CD44 antibody, an anti-CD56 antibody, an anti-CD70 antibody, an anti-CD73 antibody, an anti-CD105 antibody, an anti-CEA antibody, an anti-A33 antibody, an anti-Cripto antibody, an anti-EphA2 antibody, an anti-G250 antibody, an anti-MUC1 antibody, an anti-Lewis Y antibody, an anti-TROP2 antibody, an anti-Claudin 18.2 antibody, an anti-VEGFR antibody, an anti-GPNMB antibody, an anti-Integrin antibody, an anti-PSMA antibody, an anti-Tenascin-C antibody, an anti-SLC44A4 antibody, and an anti-Mesothelin antibody.

The term “CDR” refers to a complementarity-determining region within an antibody variable sequence. There are 3 CDRs in each of the heavy chain variable regions and light chain variable regions, which are named HCDR1, HCDR2 and HCDR3 for the heavy chain variable region, or LCDR1, LCDR2 and LCDR3 for the light chain variable region. The precise amino acid sequence boundaries of the variable region CDRs of the antibodies of the present disclosure can be determined using any of many well-known schemes, including Chothia based on the three-dimensional structure of antibodies and the topology of the CDR loops (Chothia et al., (1989) Nature 342: 877-883; Al-Lazikani et al., Standard conformations for the canonical structures of immunoglobulins, Journal of Molecular Biology, 273, 927-948 (1997)), Kabat based on antibody sequence variability (Kabat et al., Sequences of Proteins of Immunological Interest, 4th edition, U.S. Department of Health and Human Services, National Institutes of Health (1987)), AbM (University of Bath), Contact (University College London), International ImMunoGeneTics database (IMGT) (1999 Nucleic Acids Research, 27, 209-212), and North CDR definition based on the affinity propagation clustering using a large number of crystal structures. The boundaries of the CDRs of the antibodies disclosed herein can be determined by one skilled in the art according to any scheme (e.g., different assignment systems or combinations) in the art.

The term “drug loading” generally refers to the average amount of cytotoxic drug loaded per ligand and may also be expressed as the ratio of cytotoxic drug to antibody, and the cytotoxic drug loading may range from 0 to 12 (e.g., 1-10) cytotoxic drugs per ligand (Ab). In the embodiments of the present application, the drug loading is expressed as Na, and exemplary values may be an average of 1, 2, 3, 4, 5, 6, 7, 8, 9 and 10. The drug loading per ADC molecule after the coupling reaction can be characterized by conventional methods such as UV/visible spectroscopy, mass spectrometry, ELISA assays and HPLC.

In the present application, the term “pharmaceutical composition” generally refers to a mixture containing one or more of the compounds described herein or a physiologically/pharmaceutically acceptable salt or pro-drug thereof and other chemical components, and other components such as physiologically/pharmaceutically acceptable carriers and excipients. The pharmaceutical composition may promote the administration to an organism and facilitate the absorption of the active ingredient, thereby exerting biological activities. The preparation of conventional pharmaceutical compositions can refer to Chinese Pharmacopoeia. The pharmaceutical composition may be in the form of a sterile aqueous injection or oily suspension for intramuscular and subcutaneous administration. The suspension may be prepared according to a known technique using those suitable dispersing agents or wetting agents and suspending agents described above. The sterile formulation for injection may also be a sterile solution for injection or suspension prepared in a parenterally acceptable non-toxic diluent or solvent, e.g., a solution prepared in 1,3-butanediol. In addition, a sterile fixed oil may be conveniently used as a solvent or a suspending medium. For example, any blended fixed oil including synthetic mono- or di-glycerides can be used. In addition, fatty acids such as oleic acid may also be used in the preparation of injections.

In the present application, the term “pharmaceutically acceptable salt” generally refers to a salt of the compound or the ligand-drug conjugate of the present application. Such salts may be safe and/or effective when used in mammals and may possess the required biological activity, and the ligand-drug conjugate disclosed herein may form a salt with an acid. Non-limiting examples of pharmaceutically acceptable salts include: hydrochloride, hydrobromide, hydroiodide, sulfate, bisulfate, citrate, acetate, succinate, ascorbate, oxalate, nitrate, sorbate, hydrogen phosphate, dihydrogen phosphate, salicylate, hydrogen citrate, tartrate, maleate, fumarate, formate, benzoate, methanesulfonate, ethanesulfonate, benzenesulfonate, and p-toluenesulfonate.

In the present application, the term “conjugate” generally refers to a compound prepared by subjecting the compounds of the present application to one or more chemical reactions or by linking the compounds to one another via one or more linking structures such as bridges, spacers, or linkers. In the present application, the term “pharmaceutically acceptable carrier” generally refers to a carrier or vehicle for providing therapeutic agents, such as antibodies or polypeptides, genes, and other therapeutic agents. The term refers to any pharmaceutical carrier that does not itself induce the production of antibodies harmful to the individual receiving the composition and can be given without producing undue toxicity. Suitable carriers may be macromolecules that are large and metabolized slowly, such as proteins, polysaccharides, polylactic acids, polyglycolic acids, poly(amino acid)s, amino acid copolymers, lipid aggregates, and inactivated virus particles. Such carriers are well known to those skilled in the art. Pharmaceutically acceptable carriers in therapeutic compositions may include liquids such as water, saline, glycerol, and ethanol. Auxiliary substances, such as wetting or emulsifying agents, or pH buffering substances, may also be present in these carriers.

In the present application, the terms “treatment” and “treating” generally refer to a method for achieving beneficial or desired outcomes, including but not limited to therapeutic benefits. Therapeutic benefits include but are not limited to eradication, inhibition, reduction, or amelioration of the underlying disorder being treated. In addition, therapeutic benefits are achieved by eradicating, inhibiting, reducing, or ameliorating one or more physiological symptoms associated with the underlying disorder, and thus improvements are observed in the patient, but the patient may still be afflicted with the underlying disorder.

In the present application, the terms “prevention” and “preventing” generally refer to a method for achieving beneficial or desired outcomes, including but not limited to prophylactic benefits. For the purpose of prophylactic benefits, a pharmaceutical composition can be administered to a patient at risk of developing a particular disease, or to a patient who reports he/she has one or more physiological symptoms of the disease, even if the patient has not yet been diagnosed with the disease. The terms “therapeutically effective amount”, “therapeutically effective dose”, and “effective amount” refer to an amount of the ligand-drug conjugate of the present disclosure that is effective in preventing or ameliorating one or more symptoms of a disease or condition or the progression of the disease or condition when administered alone or in combination with other therapeutic drugs to a cell, tissue or subject. The therapeutically effective dose also refers to a dose sufficient to cause amelioration of symptoms, e.g., an amount for treating, curing, preventing or ameliorating a related condition or promoting the treatment, cure, prevention or amelioration of such condition. When an active ingredient is administered to an individual alone, a therapeutically effective dose only refers to the amount of the ingredient. In the case of administration in combination, a therapeutically effective dose refers to the combined amount of active ingredients that produces a therapeutic effect, regardless of whether these active ingredients are administered in combination, sequentially or simultaneously. An effective amount of a therapeutic agent will result in an increase in a diagnostic index or parameter by at least 10%, generally at least 20%, preferably at least about 30%, more preferably at least 40%, and most preferably at least 50%.

In the present application, the term “subject” or “patient” generally refers to a human (i.e., a male or female in any age group, e.g., a pediatric subject (e.g., an infant, a child, or an adolescent) or an adult subject (e.g., a young adult, a middle-aged adult or a senior adult)) and/or other primates (e.g., a cynomolgus monkey or a rhesus monkey); a mammal, including commercially relevant mammals, such as cows, pigs, horses, sheep, goats, cats and/or dogs; and/or birds, including commercially relevant birds such as chickens, ducks, geese, quail and/or turkeys.

In the present application, the term “comprise” “comprising”, “contain” or “containing” is generally intended to include the explicitly specified features without excluding other elements. The terms “no less than” and “no more than” generally refer to the situations where the number itself is included. In the present application, the term “about” generally means varying by 0.5%-10% above or below the stated value, for example, varying by 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below the stated value.

### DETAILED DESCRIPTION

Other aspects and advantages of the present application will be readily apparent to those skilled in the art from the following detailed description. Only exemplary embodiments of the present application are shown and described in the following detailed description. As those skilled in the art will recognize, the content of the present application enables those skilled in the art to make changes to the specific embodiments disclosed without departing from the spirit and scope of the invention to which the present application pertains. Accordingly, descriptions in the specification of the present application are only illustrative rather than restrictive.

The embodiments of the present application are described below with reference to specific examples, and other advantages and effects of the present application will be readily apparent to those skilled in the art from the disclosure of the present specification.

### Embodiments for Synthesis

In order to complete the synthesis purpose of the present application, the compounds provided by the present application can be prepared by the following embodiments.

Reagents that provide bromination conditions include, but are not limited to, aqueous bromine, N-bromosuccinimide, 1,3-dibromo-5,5-dimethylhydantoin, phosphorus tribromide, liquid bromine, liquid bromine/triphenylphosphine, hydrobromic acid, and carbon tetrabromide.

Titanium catalysts include, but are not limited to, tetraisopropyl titanate, triisopropoxytitanium chloride, titanium tetrachloride, and triisopropoxytitanium methyl.

Palladium catalysts include, but are not limited to, tetrakis(triphenylphosphine)palladium, palladium acetate, palladium chloride, bis(triphenylphosphine)palladium(II) dichloride, tris(dibenzylideneacetone)dipalladium, bis(dibenzylideneacetone)dipalladium, bis(acetonitrile)palladium(II) dichloride, [1,1′-bis(diphenylphosphino)ferrocene]palladium(II) dichloride, [1,1′-bis(diphenylphosphino)ferrocene]palladium(II) dichloride dichloromethane complex, bis(benzonitrile)palladium chloride, 1,4-butylenebis(diphenylphosphine)-palladium dichloride, allylpalladium chloride dimer, and allyl(cyclopentadienyl)palladium(II).

Borate dimers include, but are not limited to, bis(pinacolato)diboron, bis(neopentyl glycolato)diboron, bis(hexylene glycolato)diboron, bis(catecholato)diboron, bis(diisopropyl-L-tartrate glycolato)diboron, bis[(−)pinanediol]diborate, bis[(1S,2S,3R,5S)(+)-pinanediolato]diboron, tetramethyidiborane, bis(N,N,N′,N′-tetramethyl-D-tartramidate)diboron, tetrahydroxydiboron, bis(N,N,N′,N′-tetramethyl-L-tartramidate)diboron, bis(diisopropyl-D-tartrate glycolato)diboron, bis(diethyl-D-tartrate glycolato)diboron, bis(2,4-dimethyl-2,4-pentanediol)borate, bis(diethyl-L-tartrate glycolato)diboron, and 4,4,5,5-tetramethyl-1,3,2-dioxaborolan-2-ylboronic acid.

Metallic copper salts include, but are not limited to, copper sulfate, copper sulfate pentahydrate, cuprous sulfate, cupric chloride, cuprous chloride, cupric carbonate, cupric phosphate, cupric acetate and hydrates thereof, cupric oxalate, cupric fluoroborate and hydrates thereof, cupric methoxide, cupric tartrate, copper formate, cuprous iodide, copper(II) trifluoroacetate, copper(II) trifluoromethanesulfonate, copper carbonate, cupric bromide, cuprous bromide, and cuprous oxide. Ligands may be selected from any ligand commonly used in the Ullmann reaction, including but not limited to, L-proline, tyrosine, phenylalanine, 1,10-phenanthroline, N,N′-dimethylethylenediamine, ethylene glycol, 1,1′-binaphthyl-2,2′-diol, ethyl 2-cyclohexanonecarboxylate, and salicylaldehyde hydrazone;

Condensing agents may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N-dicyclohexylcarbodiimide, N,N-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride.

Reagents that provide basic conditions include organic bases and inorganic bases, wherein the organic bases include, but are not limited to, triethylamine, diethylamine, N-methylmorpholine, pyridine, piperidine, N,N-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, sodium phosphate, and potassium phosphate.

Reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid, and trifluoroacetic acid, and the Lewis acids include, but are not limited to, boron trifluoride, zinc chloride, magnesium chloride, aluminum chloride, stannic chloride, and ferric chloride.

Hydrogenation conditions include, but are not limited to: Pb/C/hydrogen, Pt/C/hydrogen, palladium chloride/hydrogen, Raney nickel/hydrogen, palladium hydroxide carbon/hydrogen, and palladium hydroxide/hydrogen.

Reagents that provide oxidation conditions include, but are not limited to, Dess-Martin periodinane, hydrogen peroxide, sodium chlorite, sodium hypochlorite, and potassium perchlorate.

Reagents that provide reducing conditions include, but are not limited to, sodium hydride, calcium hydride, lithium hydride, lithium aluminum hydride, sodium borohydride, lithium borohydride, sodium triethylborohydride, sodium triacetoxyborohydride, and sodium cyanoborohydride.

Reagents that provide oxidation conditions include, but are not limited to, Dess-Martin periodinane, hydrogen peroxide, sodium chlorite, sodium hypochlorite, and potassium perchlorate.

Reagents that provide nitration conditions include, but are not limited to, dilute nitric acid, concentrated nitric acid, concentrated sulfuric acid/nitric acid, and nitric/acetic anhydride.

Reagents that provide hydroboration include, but are not limited to, borane-tetrahydrofuran, borane-dimethylsulfide, catecholborane, pinacolborane, 9-borabicyclo[3.3.1]nonane, diisoamylborane, dicyclohexylborane, 1,1,2-trimethylpropylborane, monochloroborane, dichloroborane, monobromoborane, and dibromoborane. Reagents that provide oxidation conditions include, but are not limited to, Dess-Martin periodinane, hydrogen peroxide, sodium chlorite, sodium hypochlorite, and potassium perchlorate.

Basic buffers are selected from the following buffers at pH 7 to 11: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above.

In addition, the structure of the compounds in the present application is determined by nuclear magnetic resonance (NMR) or mass spectrometry (MS). NMR is performed using a Quan tum-I NMR spectrometer with deuterated dimethyl sulfoxide (DMSO-D), deuterated chloroform (CDCl3), and deuterated methanol (CD3OD) as the solvents and tetramethylsilane (TMS) as the internal standard, and chemical shifts are given in units of 10_6 (ppm).

MS is performed using an Agilent 6230 ESI-TOF mass spectrometer (manufacturer: Agilent, type c: 6230).

UPLC is performed using a Waters Acquity UPLCSQD liquid chromatograph-mass spectrometer (Poroshell 120 EC-C18, 2.1 mm×50 mm, 1.9 μm chromatography column).

HPLC is performed using an Agilent 1260 high-performance liquid chromatograph (TOSOH G3000 SW SEC chromatography column).

UV detections are performed using a Thermo Nanodrop 2000 spectrophotometer.

Enzyme-linked immunoassays are performed using an EnVision microplate reader (PerkinElmer). HSGF254 or GF254 silica gel plate is adopted as a thin-layer chromatography (TLC) silica gel plate. The specification adopted by the TLC is 0.15-0.2 mm, and the specification adopted by the thin-layer chromatography for the separation and purification of products is 0.4-0.5 mm.

Yantai Yellow Sea silica gel of 200-300 mesh is generally utilized as a carrier in column chromatography.

Known starting materials of the present application can be synthesized using or according to methods known in the art, or can be purchased from companies such as ABCR GmbH & Co. KG, Acros Organics, Aldrich Chemical Company, Accela ChemBio Inc., and Darui Chemicals.

In the examples, all reactions are conducted in an argon atmosphere or a nitrogen atmosphere unless otherwise stated. The argon atmosphere or nitrogen atmosphere means that the reaction flask is connected to a balloon containing about 1 L of argon or nitrogen. The hydrogen atmosphere means that the reaction flask is connected to a balloon containing about 1 L of hydrogen.

In the examples, the solution in the reaction is an aqueous solution unless otherwise stated.

In the examples, the reaction temperature is room temperature unless otherwise stated. The room temperature is the optimum reaction temperature, which ranges from 20° C. to 30° C.

The system of eluents for column chromatography and the system of developing agents for thin-layer chromatography used for purifying compounds include: A: dichloromethane and isopropanol system, B: dichloromethane and methanol system, and C: petroleum ether and ethyl acetate system. The volume ratio of solvents is regulated according to the different polarities of the compounds, and can also be regulated by adding a small amount of triethylamine and acidic or alkaline reagent.

Some of the compounds of the present disclosure are characterized by TOF-LC/MS. TOF-LC/MS analysis is performed using an Agilent 6230 time-of-flight mass spectrometer and an Agilent 1290-Infinity ultra-high performance liquid chromatograph.

Exemplary preparation routes for the present application are as follows:

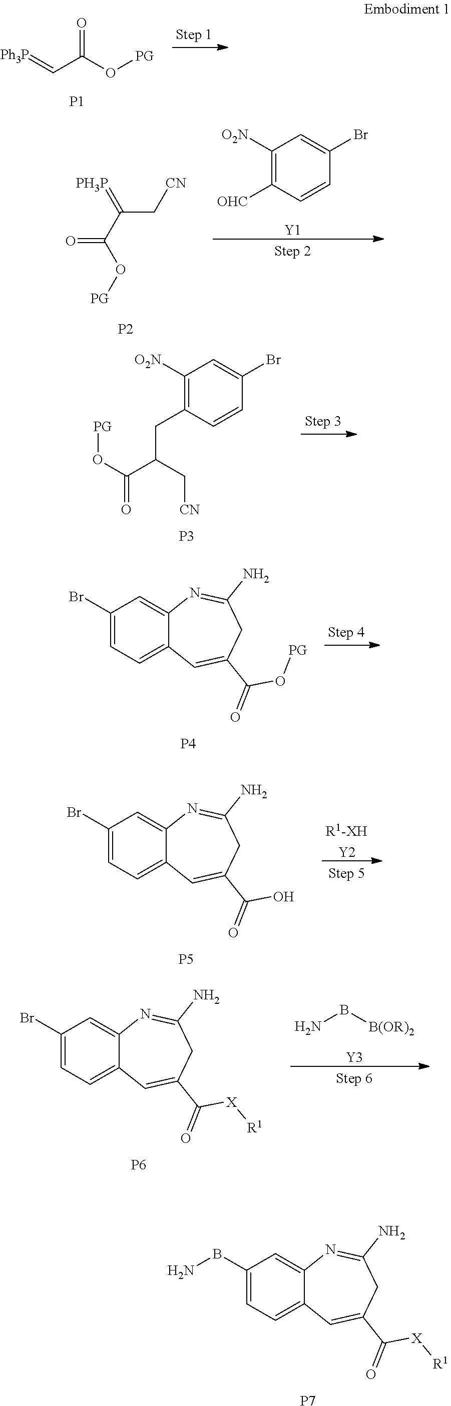

Step I: reacting a compound of general formula (P1) with bromoacetonitrile in a heating condition to give a compound of general formula (P2);
Step II: reacting the compound of general formula (P2) with a compound of general formula (Y1) at room temperature to give a compound of general formula (P3);
Step III: reacting the compound of general formula (P3) in a reducing condition to give a compound of general formula (P4);
Step IV: removing protecting group PG from the compound of general formula (P4) to give a compound of general formula (P5);
Step V: reacting the compound of general formula (P5) with a compound of general formula (Y2), optionally in the presence of a condensing agent and optionally in a basic condition, to give a compound of general formula (P6); and
Step VI: reacting the compound of general formula (P6) with a compound of general formula (Y3), optionally with the catalysis of a palladium-based reagent, to give a compound of general formula (P7);
wherein
PG may be a common carboxy protecting group;
X is selected from: —O—, —S—, and —NR—;
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy.

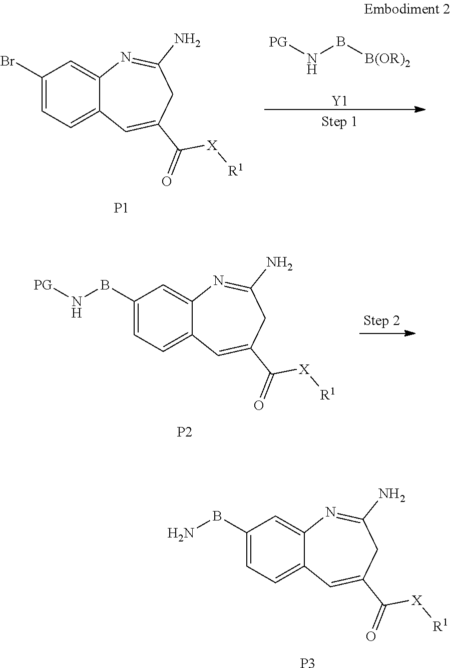

Step I: reacting a compound of general formula (P1) with a compound of general formula (YT), optionally with the catalysis of a palladium-based reagent, to give a compound of general formula (P2); and
Step II: removing protecting group PG from the compound of general formula (P2) to give a compound of general formula (P3);
wherein
PG may be a common amino protecting group;
X is selected from: —O—, —S—, and —NR—;
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy.

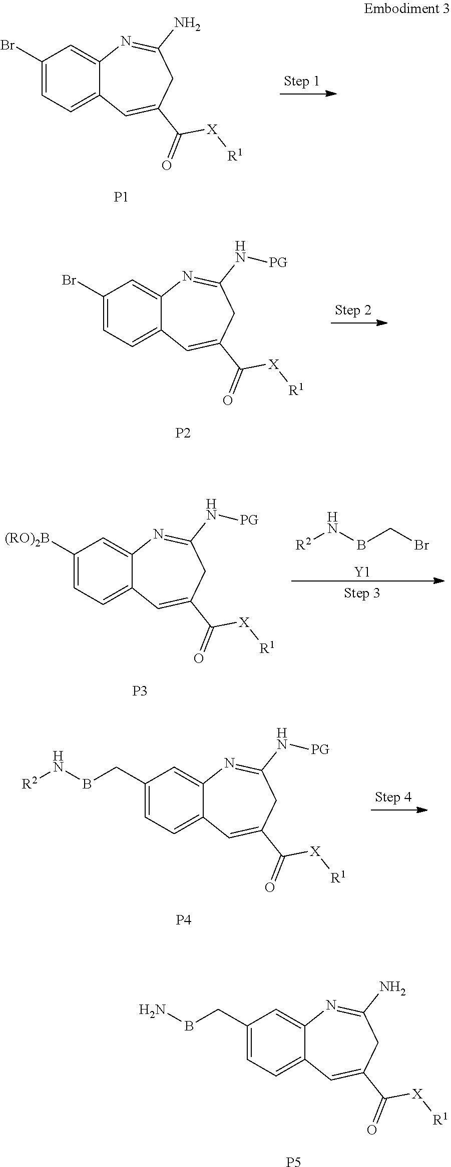

Step I: adding protecting group PG to a compound of general formula (P1), optionally in an acidic or basic condition, to give a compound of general formula (P2);
Step II: reacting the compound of general formula (P2) with a borate dimer, optionally with the catalysis of a palladium-based reagent, to give a compound of general formula (P3);
Step III: reacting the compound of general formula (P3) with a compound of general formula (Y1), optionally with the catalysis of a palladium-based reagent, to give a compound of general formula (P4); and
Step IV: removing protecting group PG from the compound of general formula (P4) to give a compound of general formula (P5);
wherein
PG may be a common amino protecting group;
R₂ may be hydrogen or a common amino protecting group;
X is selected from: —O—, —S—, and —NR—;
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy.

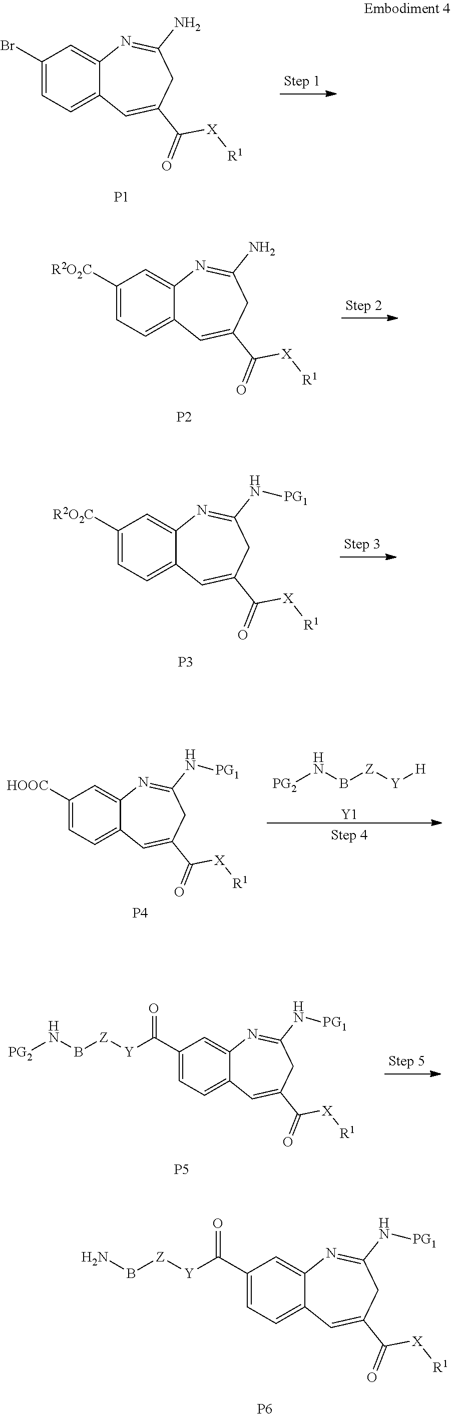

Step I: subjecting a compound of general formula (P1) to a carbonyl insertion reaction, optionally with the catalysis of a palladium-based reagent, to give a compound of general formula (P2);
Step II: adding protecting group PG₁ to the compound of general formula (P2), optionally in an acidic or basic condition, to give a compound of general formula (P3);
Step III: hydrolyzing the ester group of the compound of general formula (P3), optionally in a basic condition, to give a compound of general formula (P4);
Step IV: reacting the compound of general formula (P4) with a compound of general formula (Y1), optionally in the presence of a condensing agent and optionally in a basic condition, to give a compound of general formula (P5); and
Step V: removing protecting groups PG₁ and PG₂ from the compound of general formula (P5) to give a compound of general formula (P6);
wherein
PG₁ and PG₂ may be common amino protecting groups;
X is selected from: —O—, —S—, and —NR—;
Y is selected from: —O—, —S—, and —NR—;
R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R² is optionally selected from C₁-C₆ alkyl;
ring B is independently selected from optionally substituted aryl or heteroaryl;
Z is absent or selected from C₁-C₆ alkylene, arylene, heteroarylene, cycloalkylene, heterocycloalkylene, and halogenated C₁-C₆ alkylene.

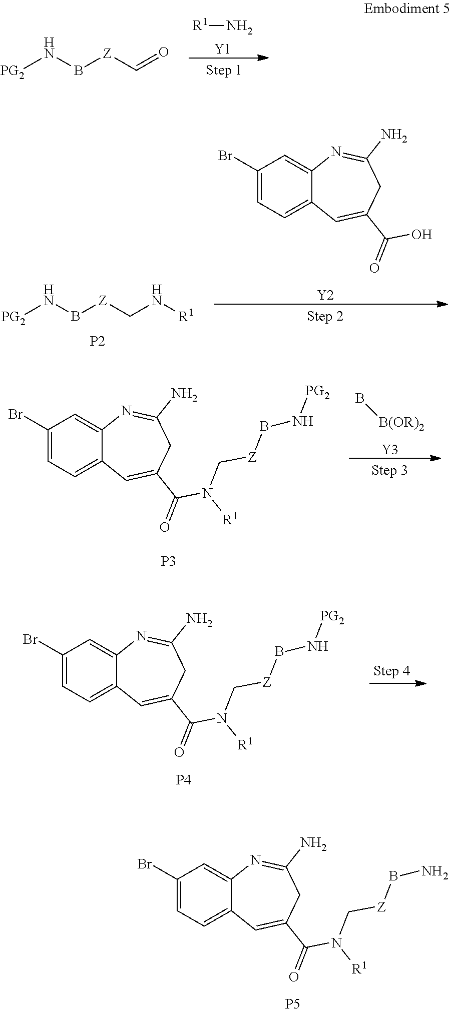

Step I: subjecting a compound of general formula (P1) and a compound of general formula (Y1) to the reductive amination reaction, optionally in a reducing condition, to give a compound of general formula (P2);
Step II: reacting the compound of general formula (P2) with a compound of general formula (Y2), optionally in the presence of a condensing agent and optionally in a basic condition, to give a compound of general formula (P3);
Step III: reacting the compound of general formula (P3) with a compound of general formula (Y3), optionally with the catalysis of a palladium-based reagent, to give a compound of general formula (P4); and
Step IV: removing protecting group PG from the compound of general formula (P4) to give a compound of general formula (P5);
wherein
PG may be a common amino protecting group;
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
Z is absent or selected from C₁-C₆ alkylene, arylene, heteroarylene, cycloalkylene, heterocycloalkylene, and halogenated C₁-C₆ alkylene.

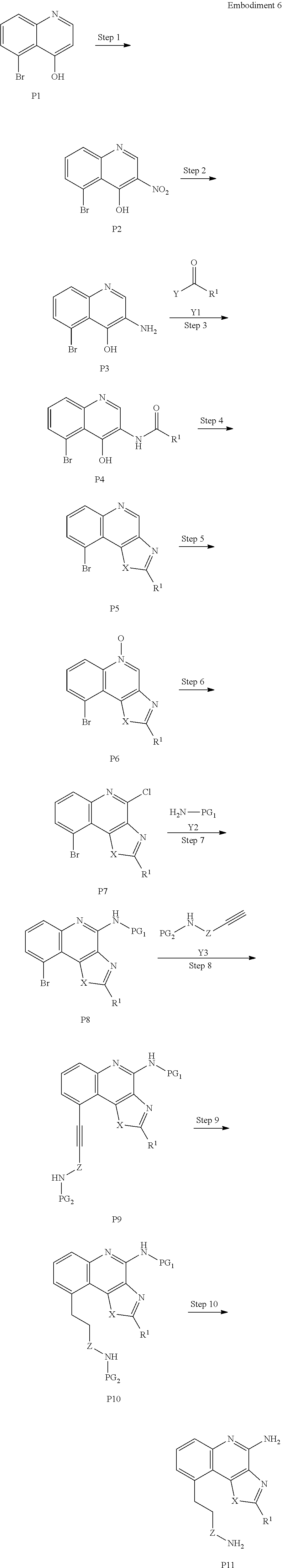

Step I: subjecting a compound of general formula (P1) to a nitration condition to give a compound of general formula (P2);
Step II: subjecting the compound of general formula (P2) to a reducing condition to give a compound of general formula (P3);
Step III: reacting the compound of general formula (P3) with a compound of general formula (Y1), optionally in the presence of a condensing agent and optionally in a basic condition, to give a compound of general formula (P4);
Step IV: subjecting the compound of general formula (P4) to cyclization reaction in a heating condition to give a compound of general formula (P5);
Step V: subjecting the compound of general formula (P5) to an oxidation condition to give a compound of general formula (P6);
Step VI: reacting the compound of general formula (P6) in the presence of phosphorus oxychloride to give a compound of general formula (P7);
Step VII: reacting the compound of general formula (P7) with a compound of general formula (Y2) in a heating condition to give a compound of general formula (P8);
Step VIII: reacting the compound of general formula (P8) with a compound of general formula (Y3), optionally with the catalysis of a palladium-based reagent, to give a compound of general formula (P9);
Step IX: reacting the compound of general formula (P9) in a reducing condition to give a compound of general formula (P10); and
Step X: removing protecting groups PG₁ and PG₂ from the compound of general formula (P10) to give a compound of general formula (P11);
wherein
PG₁ and PG₂ may be common amino protecting groups;
X is selected from: —O— and —S—;
Z is absent or selected from C₁-C₆ alkylene, arylene, heteroarylene, cycloalkylene, heterocycloalkylene, and halogenated C₁-C₆ alkylene.
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy; wherein when R¹ comprises methylene units, the methylene units of R¹ are each independently unreplaced, or the methylene units of R¹ are each independently replaced by a group selected from the group consisting of: —O—, —S—, and optionally substituted —NH—.

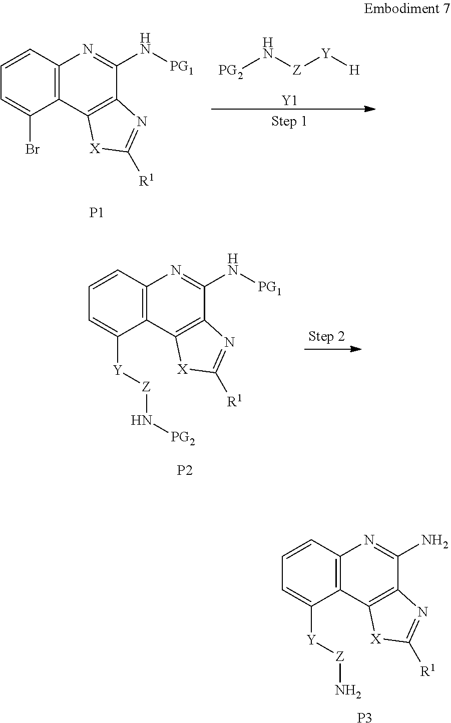

Step I: reacting a compound of general formula (P1) with a compound of general formula (Y1), optionally with the catalysis of a palladium-based reagent, to give a compound of general formula (P2); and
Step II: removing protecting groups PG₁ and PG₂ from the compound of general formula (P2) to give a compound of general formula (P3);
wherein
PG₁ and PG₂ may be common amino protecting groups;
X is selected from: —O— and —S—;
Y is selected from: —O— and —NH—;
Z is absent or selected from C₁-C₆ alkylene, arylene, heteroarylene, cycloalkylene, heterocycloalkylene, and halogenated C₁-C₆ alkylene.
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy; wherein when R¹ comprises methylene units, the methylene units of R¹ are each independently unreplaced, or the methylene units of R¹ are each independently replaced by a group selected from the group consisting of: —O—, —S—, and optionally substituted —NH—.

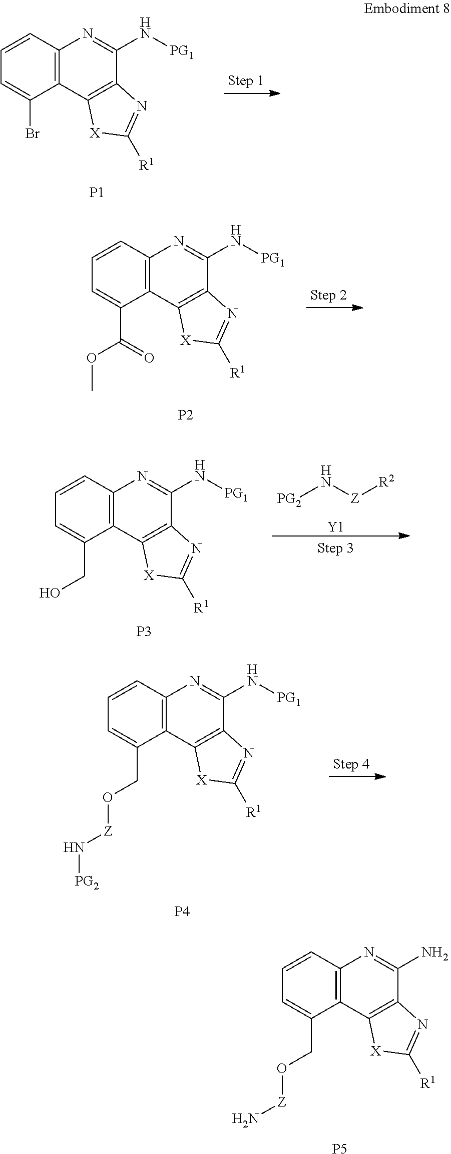

Step I: subjecting a compound of general formula (P1) to a carbonyl insertion reaction, optionally with the catalysis of a palladium-based reagent, to give a compound of general formula (P2);
Step II: subjecting the compound of general formula (P2) to a reducing condition to give a compound of general formula (P3);
Step III: subjecting the compound of general formula (P3) and a compound of general formula (Y1) to substitution reaction in a basic condition to give a compound of general formula (P4); and
Step IV: removing protecting groups PG₁ and PG₂ from the compound of general formula (P4) to give a compound of general formula (P5);
wherein
PG₁ and PG₂ may be common amino protecting groups;
X is selected from: —O— and —S—;
R² is selected from: halogen, methanesulfonyloxy, and p-toluenesulfonyloxy;
Z is absent or selected from C₁-C₆ alkylene, arylene, heteroarylene, cycloalkylene, heterocycloalkylene, and halogenated C₁-C₆ alkylene.
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy; wherein when R^{t} comprises methylene units, the methylene units of R¹ are each independently unreplaced, or the methylene units of R¹ are each independently replaced by a group selected from the group consisting of: —O—, —S—, and optionally substituted —NH—.

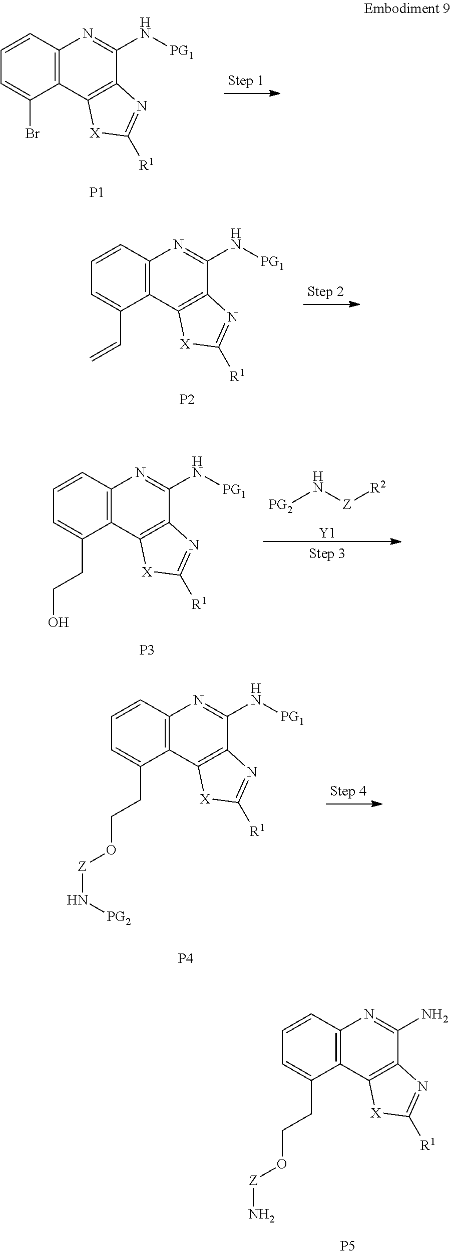

Step I: subjecting a compound of general formula (P1) to a Heck reaction, optionally with the catalysis of a palladium-based reagent, to give a compound of general formula (P2);
Step II: subjecting the compound of general formula (P2) to hydroboration and oxidation conditions to give a compound of general formula (P3);
Step III: subjecting the compound of general formula (P3) and a compound of general formula (Y1) to substitution reaction in a basic condition to give a compound of general formula (P4); and
Step IV: removing protecting groups PG₁ and PG₂ from the compound of general formula (P4) to give a compound of general formula (P5);
wherein
PG₁ and PG₂ may be common amino protecting groups;
X is selected from: —O— and —S—;
R² is selected from: halogen, methanesulfonyloxy, and p-toluenesulfonyloxy;
Z is absent or selected from C₁-C₆ alkylene, arylene, heteroarylene, cycloalkylene, heterocycloalkylene, and halogenated C₁-C₆ alkylene.
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy; wherein when R¹ comprises methylene units, the methylene units of R¹ are each independently unreplaced, or the methylene units of R¹ are each independently replaced by a group selected from the group consisting of: —O—, —S—, and optionally substituted —NH—.

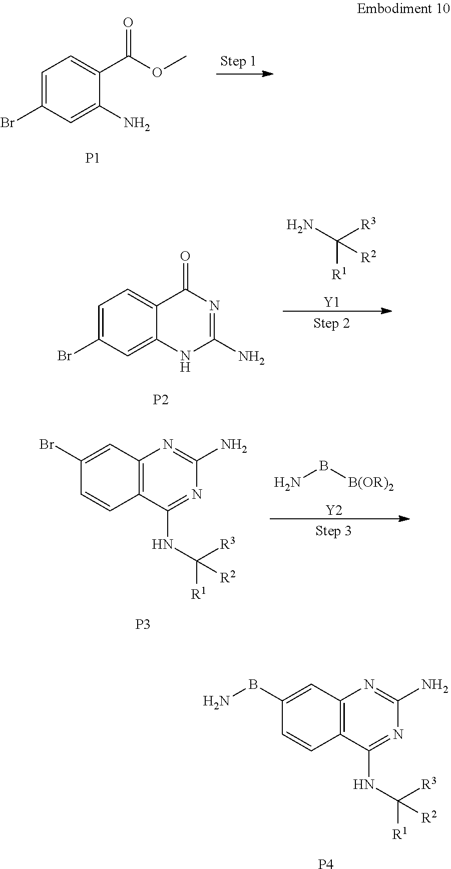

Step I: reacting a compound of general formula (P1) with aminoacetonitrile in a heating condition to give a compound of general formula (P2);
Step II: reacting the compound of general formula (P2) with a compound of general formula (Y1), optionally in the presence of a condensing agent and optionally in a basic condition, to give a compound of general formula (P3); and
Step III: reacting the compound of general formula (P3) with a compound of general formula (Y2), optionally with the catalysis of a palladium-based reagent, to give a compound of general formula (P4);
wherein
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹, R², and R³ are independently selected from hydrogen, protium, deuterium, tritium, optionally substituted C₁-C₆ alkyl, optionally substituted C₁-C₆ alkoxy, optionally substituted aryl, optionally substituted heteroaryl, optionally substituted cycloalkyl, and optionally substituted heterocycloalkyl.

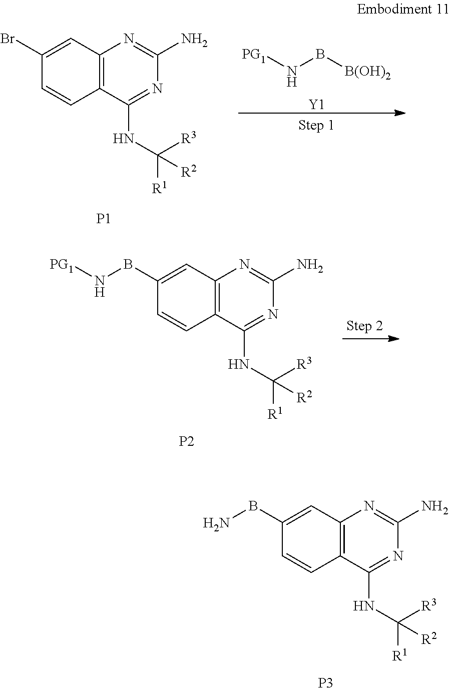

Step I: reacting a compound of general formula (P1) with a compound of general formula (YT), optionally with the catalysis of a palladium-based reagent, to give a compound of general formula (P2); and
Step II: removing protecting groups PG₁ and PG₂ from the compound of general formula (P2) to give a compound of general formula (P3);
wherein
PG₁ may be a common amino protecting group;
ring B is optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹, R², and R³ are independently selected from hydrogen, protium, deuterium, tritium, optionally substituted C₁-C₆ alkyl, optionally substituted C₁-C₆ alkoxy, optionally substituted aryl, optionally substituted heteroaryl, optionally substituted cycloalkyl, and optionally substituted heterocycloalkyl.

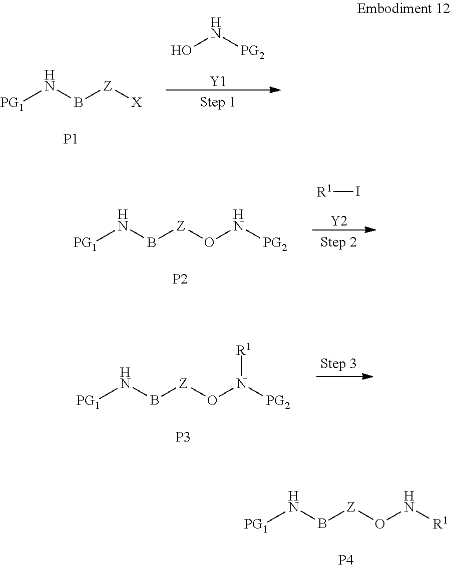

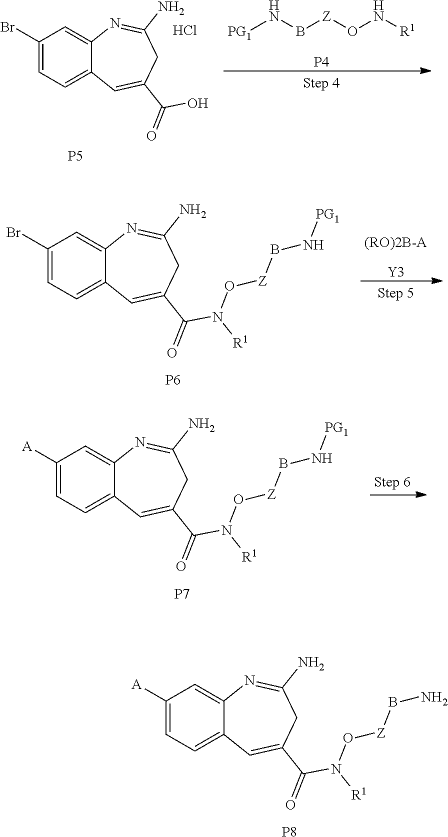

Step I: reacting a compound of general formula (P1) with a compound of general formula (Y1), optionally in a basic condition, to give a compound of general formula (P2);
Step II: reacting the compound of general formula (P2) with a compound of general formula (Y2), optionally in a basic condition, to give a compound of general formula (P3);
Step III: removing protecting group PG₂ from the compound of general formula (P3) to give a compound of general formula (P4);
Step IV: reacting a compound of general formula (P5) with the compound of general formula (P4), optionally in the presence of a condensing agent and optionally in a basic condition, to give a compound of general formula (P6);
Step V: reacting the compound of general formula (P6) with a compound of general formula (Y3), optionally with the catalysis of a palladium-based reagent, to give a compound of general formula (P7); and
Step VI: removing protecting group PG₁ from the compound of general formula (P7) to give a compound of general formula (P8);
wherein
PG₁ and PG₂ may be common amino protecting groups;
X is independently selected from: halogen, methanesulfonyloxy, and p-toluenesulfonyloxy; ring A and ring B are independently selected from optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is optionally substituted C₁-C₆ alkyl;
Z is selected from optionally substituted C₁-C₆ alkylene, optionally substituted cycloalkylene, and optionally substituted heterocycloalkylene.

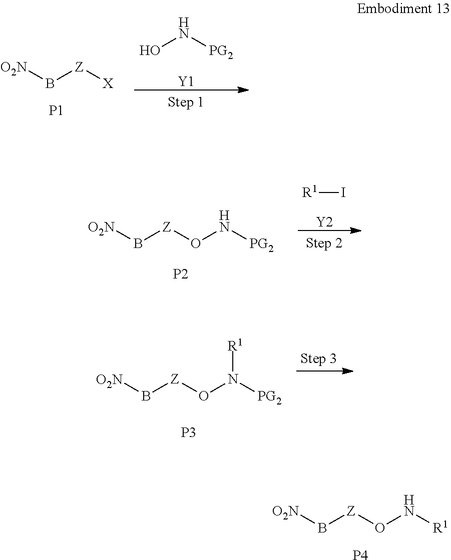

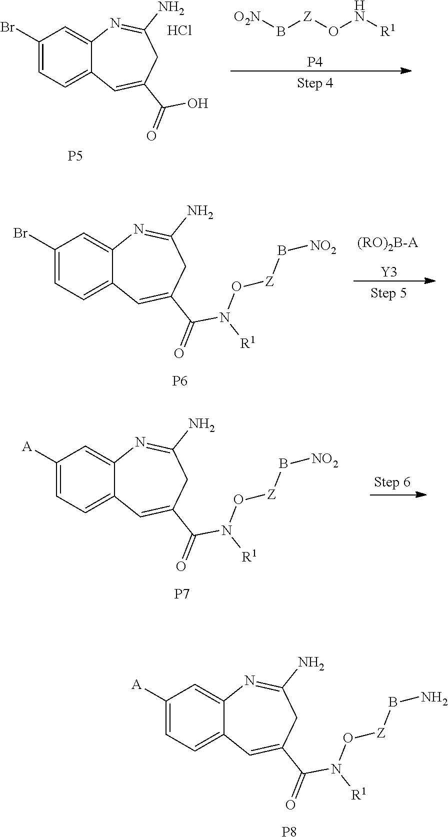

Step I: reacting a compound of general formula (P1) with a compound of general formula (Y1), optionally in a basic condition, to give a compound of general formula (P2);
Step II: reacting the compound of general formula (P2) with a compound of general formula (Y2), optionally in a basic condition, to give a compound of general formula (P3);
Step III: removing protecting group PG₂ from the compound of general formula (P3) to give a compound of general formula (P4);
Step IV: reacting a compound of general formula (P5) with the compound of general formula (P4), optionally in the presence of a condensing agent and optionally in a basic condition, to give a compound of general formula (P6);
Step V: reacting the compound of general formula (P6) with a compound of general formula (Y3), optionally with the catalysis of a palladium-based reagent, to give a compound of general formula (P6); and
Step VI: removing protecting group PG₁ from the compound of general formula (P4) to give a compound of general formula (P7);
wherein
PG₂ may be a common amino protecting group;
X is independently selected from: halogen, methanesulfonyloxy, and p-toluenesulfonyloxy; ring A and ring B are independently selected from optionally substituted aryl or heteroaryl;
—B(OR)₂ is a boronate monomer, wherein the two R may be connected to form a heterocycle, bridged heterocycle or spiro heterocycle that may be optionally substituted with C₁-C₆ alkyl, aryl, heteroaryl, carboxy, or acyloxy C₁-C₆ alkyl;
wherein each R is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R¹ is optionally substituted C₁-C₆ alkyl;
Z is selected from optionally substituted C₁-C₆ alkylene, optionally substituted cycloalkylene, and optionally substituted heterocycloalkylene.

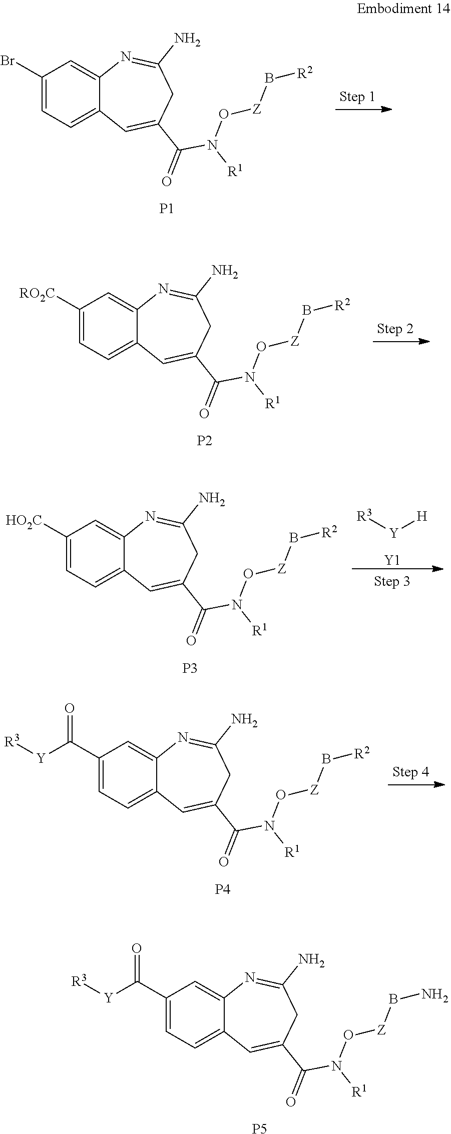

Step I: subjecting a compound of general formula (P1) to a carbonyl insertion reaction, optionally with the catalysis of a palladium-based reagent, to give a compound of general formula (P2);
Step II: hydrolyzing the ester group of the compound of general formula (P2), optionally in a basic condition, to give a compound of general formula (P3);
Step III: reacting the compound of general formula (P3) with a compound of general formula (Y1), optionally in the presence of a condensing agent and optionally in a basic condition, to give a compound of general formula (P4);
Step IV: reducing the nitro group of the compound of the general formula (P4) in a reducing condition, or removing protecting group PG optionally in a basic or acidic condition, to give a compound of the general formula (P5);
wherein
ring B is optionally substituted aryl or heteroaryl;
Z is selected from optionally substituted C₁-C₆ alkylene, optionally substituted cycloalkylene, and optionally substituted heterocycloalkylene;
R¹ is independently selected from hydrogen, protium, deuterium, tritium, oxygen, hydroxy, halogen, C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy;
R² is optionally selected from nitro or —NH-PG, wherein PG may be a common amino protecting group;
R is optionally selected from C₁-C₆ alkyl;
Y is selected from: —O—, —S—, and —NR—;
R³ is optionally selected from C₁-C₆ alkyl, C₁-C₆ alkoxy, aryl, heteroaryl, cycloalkyl, heterocycloalkyl, halogenated C₁-C₆ alkyl, and halogenated C₁-C₆ alkoxy.

Without being bound by any theory, the following examples are intended only to illustrate the compounds, preparation methods, use, etc., of the present application, and are not intended to limit the scope of the present application.

### EXAMPLES

### Example 1

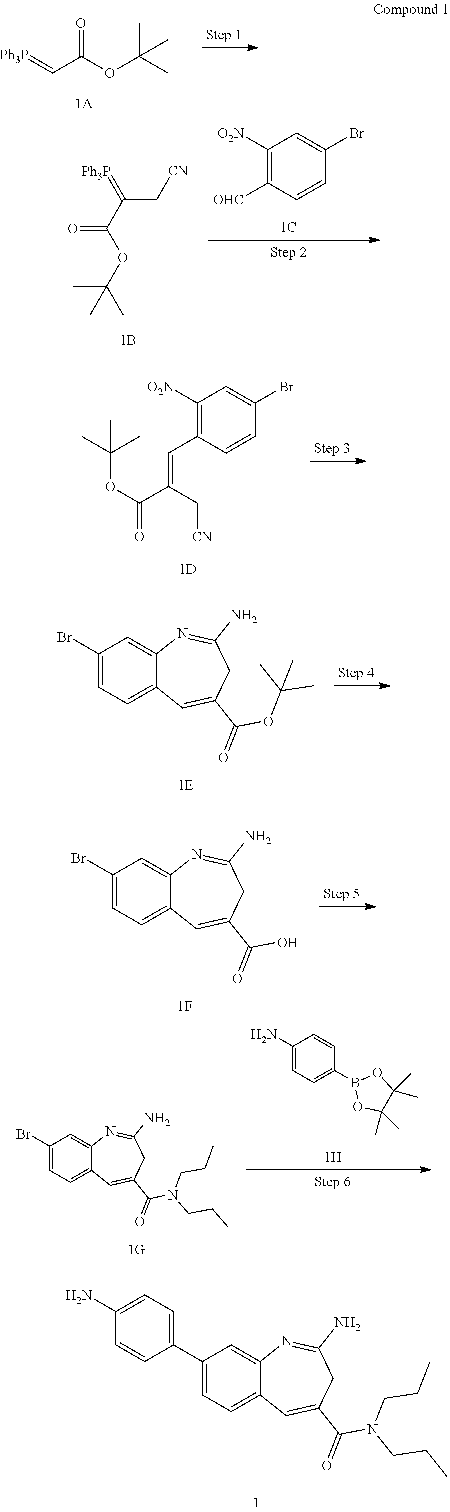

Step 1 EA (180 mL) and 1A (30 g, 79.7 mmol, 1 eq) were added into a 500-mL three-neck flask. Bromoacetonitrile (5.73 g, 47.7 mmol, 0.6 eq) was dropwise added at room temperature. After the dropwise addition, the system was heated to 85° C. and incubated for reaction overnight. The reaction solution was cooled and directly filtered. The filter cake was washed with 50 mL of EA, and the filtrate was concentrated by rotary evaporation to give a crude dark red solid product 1B (9 g), which was directly used in the next step without calculating the yield.

Step 2 1B (9 g, 21.74 mmol, 1 eq) from the previous step was dissolved (incompletely) in toluene (90 mL). 1C (5 g, 21.74 mmol, 1 eq) was added at room temperature. After the addition, the system was stirred at room temperature overnight. Toluene was removed by rotary evaporation. The residue was subjected to column chromatography (PE:EA=20:1 to 10:1), concentrated by rotary evaporation at reduced pressure, triturated in PE (50 mL), and filtered to give a pale yellow flaky crystal 1D (5 g, 62.5% yield).

Step 3 In a 250-mL three-neck flask, 1D (5 g, 13.6 mmol, 1 eq) was added to glacial acetic acid (50 mL). The internal temperature was raised to around 60-65° C., before iron powder (3.8 g, 68 mmol, 5 eq) was slowly added in portions. After the addition, the internal temperature was raised to 85° C. for reaction for 2 h. The reaction solution was cooled to around 60° C., diluted by adding DCM (50 mL), and filtered through celite before cooling. The filter cake was washed with DCM (100 mL), and the filtrate was concentrated by rotary evaporation at reduced pressure to give a dark red oily substance. The oil was diluted with DCM (50 mL), poured into saturated aqueous sodium bicarbonate (200 mL), and extracted with DCM (100 mL×4). The organic phases were combined, dried over sodium sulfate, and concentrated at reduced pressure. After concentration by rotary evaporation, the mixture was triturated in PE:EA=2:1 (30 mL) and filtered to give an off-white powder 1E (4.5 g, 97% yield). MS-ESI: m/z 337.1 [M+H]⁺.

Step 4 4 N HCl/dioxane (40 mL) was added into a 100-mL single-neck flask, before 1E (4.5 g, 13.4 mmol, 1 eq) was added. The system was stirred at room temperature overnight. When LCMS indicated the depletion of the starting materials, the mixture was directly concentrated by rotary evaporation to give an off-white powder 1F (4.2 g, 99% yield). MS-ESI: m/z 281.0 [M+H]⁺.

Step 5 DMF (60 mL) and 1F (3 g, 9.45 mmol, 1 eq) were added into a 250-mL three-neck flask, before HBTU (4.3 g, 11.34 mmol, 1.2 eq) and DIEA (4.88 g, 37.79 mmol, 4 eq) were added at 0° C. After the addition, the system was stirred for 10 min, dipropylamine (1.86 g, 18.42 mmol, 1.95 eq) was dropwise added. After the dropwise addition, the system was warmed to room temperature and stirred overnight. The reaction solution was poured into water (240 mL), and extracted with EA (100 mL×5). The organic phase was washed with saturated brine (50 mL×2), dried over sodium sulfate, concentrated by rotary evaporation, mixed with silica gel, and subjected to column chromatography to give a dark red solid 1G (3 g, 87.5% yield). MS-ESI: m/z 364.2 [M+H]⁺.

Step 6 In a 25-mL three-neck flask, 1G (200 mg, 0.55 mmol, 1 eq), 1H (121 mg, 0.55 mmol, 1 eq) and anhydrous potassium phosphate (350 mg, 1.65 mmol, 3 eq) were dissolved in a mixed solvent of dioxane (5 mL) and water (0.5 mL). The system was purged twice with argon before Pd(PPh₃)₄ (50 mg) was added, then purged twice again with argon, heated to 85° C., and incubated for reaction for 3 h. After cooling, the reaction solution was dried over sodium sulfate to remove water and directly purified by thin-layer chromatography to give a dark yellow powder 1 (25 mg, 12% yield). MS-ESI: m/z 377.3 [M+H]⁺.

¹H NMR (400 MHz, DMSO-d₆) δ 7.36 (d, J=8.6 Hz, 2H), 7.25 (d, J=8.2 Hz, 1H), 7.15 (d, J=1.9 Hz, 1H), 7.11 (dd, J=8.2, 2.0 Hz, 1H), 6.79-6.67 (m, 3H), 6.62 (d, J=8.6 Hz, 2H), 5.22 (s, 2H), 3.30-3.25 (m, 4H), 2.70 (s, 2H), 1.63-1.48 (m, 4H), 0.82 (brs, 6H).

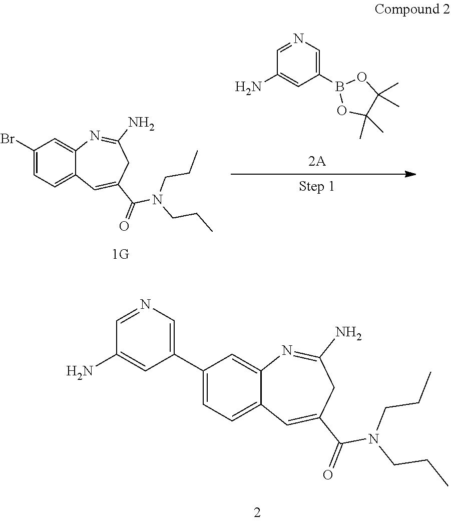 [SMILES: CCCN(CCC)C(=O)C1=Cc2ccc(Br)cc2N=C(N)C1 ; CC1(C)OB(c2cncc(N)c2)OC1(C)C ; CCCN(CCC)C(=O)C1=Cc2ccc(-c3cncc(N)c3)cc2N=C(N)C1]

Step 1 In a 25-mL three-neck flask, 1G (200 mg, 0.55 mmol, 1 eq), 2A (121 mg, 0.55 mmol, 1 eq) and anhydrous potassium phosphate (350 mg, 1.65 mmol, 3 eq) were dissolved in a mixed solvent of dioxane (5 mL) and water (0.5 mL). The system was purged twice with argon before Pd(PPh₃)₄ (50 mg) was added, then purged twice again with argon, heated to 85° C., and incubated for reaction for 3 h. After cooling, the reaction solution was dried over sodium sulfate to remove water and purified by column chromatography (DCM:MeOH=100:0 to 10:1) to give a pale yellow powder 2 (23 mg, 110% yield).

MS-ESI: m/z 378.3 [M+H]⁺.

¹H NMR (400 MHz, DMSO-d₆) δ 8.04 (d, J=2.0 Hz, 1H), 7.93 (d, J=2.5 Hz, 1H), 7.44 (d, J=8.0 Hz, 1H), 7.34-7.23 (m, 2H), 7.16 (t, J=2.3 Hz, 1H), 6.82 (s, 1H), 5.43 (s, 2H), 3.31-3.26 (m, 4H), 2.89 (s, 2H), 1.61-1.51 (m, 4H), 0.83 (brs, 6H).

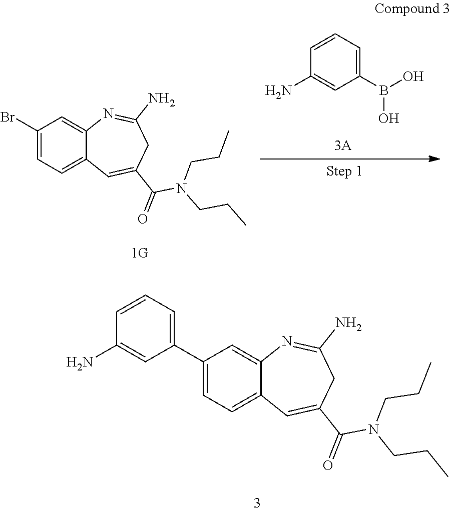 [SMILES: CCCN(CCC)C(=O)C1=Cc2ccc(Br)cc2N=C(N)C1 ; Nc1cccc(B(O)O)c1 ; CCCN(CCC)C(=O)C1=Cc2ccc(-c3cccc(N)c3)cc2N=C(N)C1]

Step 1 In a 25-mL three-neck flask, 1G (400 mg, 1.1 mmol, 1 eq), 3A (151 mg, 1.1 mmol, 1 eq) and anhydrous potassium phosphate (700 mg, 3.29 mmol, 3 eq) were dissolved in a mixed solvent of dioxane (10 mL) and water (1 mL). The system was purged twice with argon before Pd(PPh₃)₄ (100 mg) was added, then purged twice again with argon, heated to 85° C., and incubated for reaction for 3 h. After cooling, the reaction solution was dried over 5 g of sodium sulfate, directly mixed with silica gel, and subjected to column chromatography to give a crude product. The crude product was then purified to give a pale yellow powder 3 (45 mg, 11% yield).

MS-ESI: m/z 377.2 [M+H]⁺.

¹H NMR (400 MHz, Methanol-d₄) δ 7.72-7.60 (m, 3H), 7.59-7.37 (m, 3H), 7.37-7.12 (m, 1H), 7.08 (s, 1H), 3.47 (brs, 4H), 3.36 (s, 2H), 1.77-1.63 (m, 4H), 0.95 (brs, 6H).

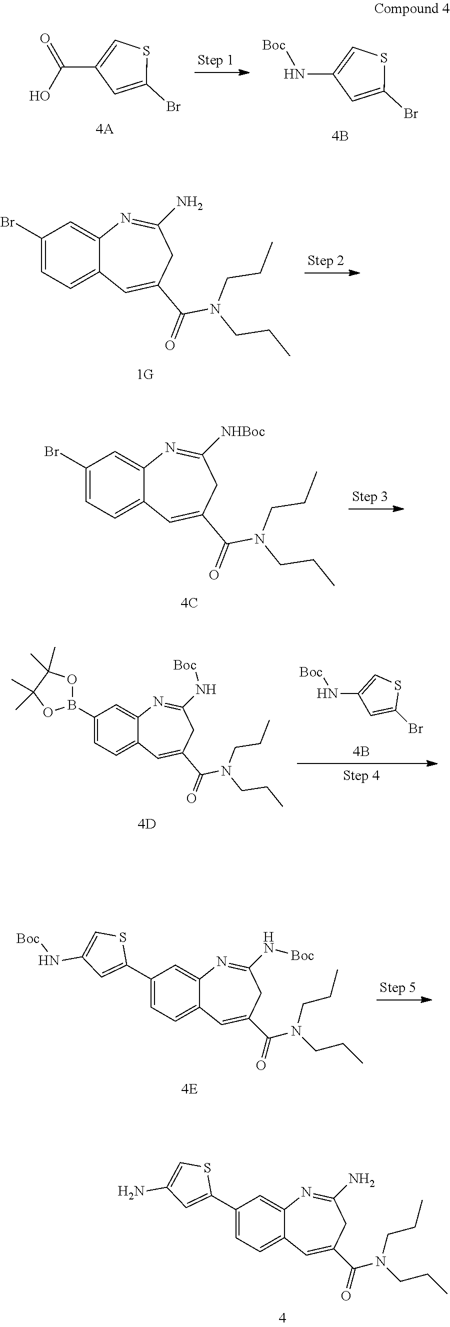

Step 1 4A (2 g, 9.66 mmol, 1 eq) was dissolved in toluene (40 mL), before triethylamine (2.44 g, 24.15 mmol, 2.5 eq), DPPA (3.19 g, 11.59 mmol, 1.2 eq), and tert-butanol (7.16 g, 96.6 mmol, 10 eq) were added at room temperature. After the addition, the system was heated to 100° C. and stirred overnight. The reaction solution was poured into 50 mL of water and extracted with EA (50 mL×3). The organic phase was dried over sodium sulfate, concentrated at reduced pressure, mixed with silica gel, and subjected to column chromatography (PE:EA=5:1) to give an off-white solid 4B (0.6 g, 22.3% yield).

Step 2 1G (2 g, 5.49 mmol, 1 eq) was dissolved in DCM (40 mL), before triethylamine (1.67 g, 16.47 mmol, 3 eq) was added at room temperature. Boc₂O (1.8 g, 8.24 mmol, 1.5 eq) was then dropwise added, and after the addition, the system was stirred at room temperature overnight. The reaction solution was poured into 50 mL of water and extracted with DCM (50 mL×3). The organic phase was dried over sodium sulfate, concentrated at reduced pressure, mixed with silica gel, and subjected to column chromatography (PE:EA=5:1) to give apale yellow solid 4C (1.2 g, 49% yield).

Step 3 4C (1.2 g, 2.58 mmol, 1 eq) was dissolved in dioxane (20 mL), before bis(pinacolato)diboron (722 mg, 2.84 mmol, 1.1 eq) and AcOK (507 mg, 5.17 mmol, 2 eq) were added. The system was purged twice with argon before Pd(dppf)Cl₂ (0.2 g) was added in one portion, then purged twice again with argon, heated to 85° C., and incubated for reaction for 3 h. When LCMS indicated the depletion of the starting materials, the reaction solution was cooled to room temperature and directly poured into a silica gel column. The product was eluted with PE:EA=2:1 to give a yellow solid 4D (600 mg, 45% yield). MS-ESI: m/z 512.3 [M+H]⁺.

Step 4 4B (359 mg, 1.29 mmol, 1.2 eq) and 4D (550 mg, 1.08 mmol, 1 eq) were added to a mixed solvent of dioxane (10 mL) and water (1 mL), before sodium carbonate (285 mg, 2.69 mmol, 2.5 eq) was added. The system was purged twice with argon before Pd(dppf)Cl₂ (100 mg) was added in one portion, then purged twice again with argon, heated to 85° C., and incubated for reaction for 3 h. When LCMS indicated the depletion of the starting materials, the reaction solution was cooled to room temperature, dried over sodium sulfate, directly mixed with silica gel, and purified by column chromatography to give a yellow solid 4E (100 mg, 15.9% yield). MS-ESI: m/z 583.3 [M+H]⁺.

Step 5 4E (100 mg, 0.17 mmol, 1 eq) was dissolved in EA (5 mL), before 4 N dioxane hydrochloride (1 mL) was added in an ice-water bath. The system was stirred at room temperature overnight. The mixture was concentrated at reduced pressure, subjected to preparative chromatography, and lyophilized to give a yellow solid 4 (22 mg, 27% yield).

MS-ESI: m/z 383.2 [M+H]⁺.

¹H NMR (400 MHz, Methanol-d₄) δ 7.35 (d, J=1.7 Hz, 1H), 7.30 (d, J=8.1 Hz, 1H), 7.26 (dd, J=8.2, 1.8 Hz, 1H), 7.05 (d, J=1.5 Hz, 1H), 6.81 (s, 1H), 6.25 (d, J=1.6 Hz, 1H), 4.52-4.38 (m, 2H), 3.46-3.37 (m, 4H), 1.73-1.60 (m, 4H), 0.91 (brs, 6H).

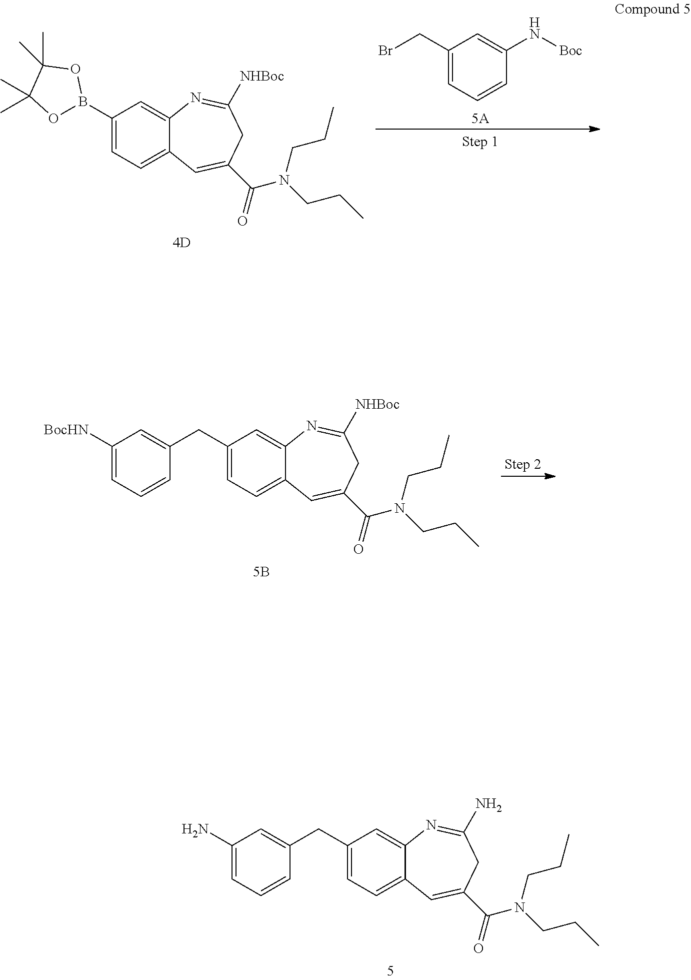 [SMILES: CCCN(CCC)C(=O)C1=Cc2ccc(B3OC(C)(C)C(C)(C)O3)cc2N=C(NC(=O)OC(C)(C)C)C1 ; CC(C)(C)OC(=O)Nc1cccc(CBr)c1 ; CCCN(CCC)C(=O)C1=Cc2ccc(Cc3cccc(NC(=O)OC(C)(C)C)c3)cc2N=C(NC(=O)OC(C)(C)C)C1 ; CCCN(CCC)C(=O)C1=Cc2ccc(Cc3cccc(N)c3)cc2N=C(N)C1]

Step 1 5A (280 mg, 0.98 mmol, 1 eq) and 4D (500 mg, 0.98 mmol, 1 eq) were added to a mixed solvent of dioxane (10 mL) and water (2 mL), before sodium carbonate (259 mg, 2.44 mmol, 2.5 eq) was added. The system was purged twice with argon before Pd(dppf)Cl₂ (100 mg) was added in one portion, then purged twice again with argon, heated to 95° C., and incubated for reaction for 3 h. When LCMS indicated the depletion of the starting materials, the reaction solution was cooled to room temperature, dried over sodium sulfate, and directly purified by thin-layer chromatography to give a yellow solid 5B (200 mg, 35% yield). MS-ESI: m/z 591.4 [M+H]⁺.

Step 2 5B (200 mg, 0.33 mmol, 1 eq) was dissolved in EA (5 mL), before 4 N dioxane hydrochloride (0.5 mL) was added in an ice-water bath. The reaction system was stirred at room temperature overnight. When LCMS indicated the depletion of the starting materials, the mixture was concentrated at reduced pressure, subjected to preparative chromatography, and lyophilized to give a yellow solid 5 (25 mg, 19% yield).

MS-ESI: m/z 391.3 [M+H]⁺.

¹H NMR (400 MHz, Methanol-d₄) δ 7.20 (d, J=7.9 Hz, 1H), 7.05-6.96 (m, 2H), 6.90 (dd, J=8.0, 1.9 Hz, 1H), 6.78 (s, 1H), 6.63-6.53 (m, 3H), 4.69-4.48 (m, 2H), 3.85 (s, 2H), 3.45-3.36 (m, 4H), 1.75-1.57 (m, 4H), 0.90 (brs, 6H).

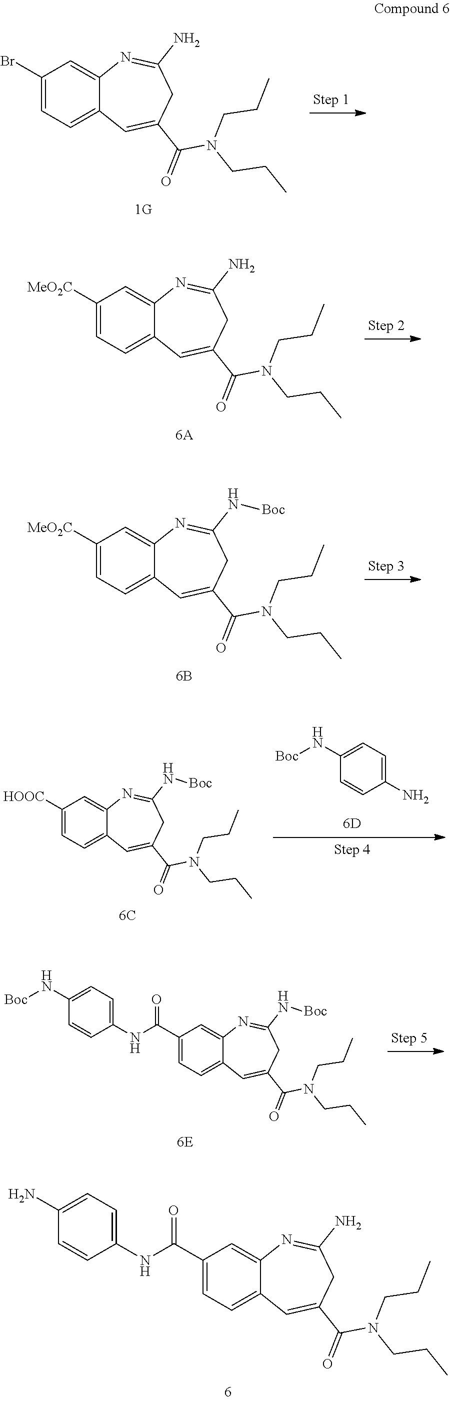

Step 1 Methanol (20 mL), 1G (1.2 g, 3.29 mmol, 1 eq), and triethylamine (1 g, 9.88 mmol, 3 eq) were added to a 50-mL autoclave. The system was purged with argon before Pd(dppf)Cl₂ (0.2 g) was added, then sealed soon after the addition, purged twice with argon before CO (60 psi) was introduced, heated to 80° C., and incubated for reaction for 40 h. When LCMS indicated a small amount of remaining starting materials, the mixture was filtered and concentrated at reduced pressure to give a crude dark red oil product 6A (1.5 g), which was directly used in the next step without calculating the yield. MS-ESI: m/z 344.2 [M+H]⁺.

Step 2 The crude product 6A (1.5 g, 4.37 mmol, 1 eq) from the previous step was dissolved in DCM (30 mL), before triethylamine (1.33 g, 13.10 mmol, 3 eq) was added at room temperature. Boc₂O (1.43 g, 6.55 mmol, 1.5 eq) was then dropwise added, and after the addition, the system was stirred at room temperature overnight. The reaction solution was poured into water (50 mL) and extracted with DCM (50 mL×3). The organic phase was dried over sodium sulfate, concentrated at reduced pressure, and subjected to column chromatography (PE:EA=5:1) to give a pale yellow solid 6B (0.4 g, 27.6% yield over the two steps). MS-ESI: m/z 444.3 [M+H]⁺.

Step 3 6B (400 mg, 0.9 mmol, 1 eq) was added to a mixed solvent of water (5 mL) and THE (5 mL), before lithium hydroxide monohydrate (56.77 mg, 1.35 mmol, 1.5 eq) was added at room temperature. The system was stirred at room temperature overnight. When TLC indicated the depletion of the starting materials, the reaction solution was adjusted to pH 5 with saturated citric acid, and extracted with ethyl acetate (10 mL×2). The organic phases were combined, dried over sodium sulfate, and concentrated at reduced pressure to give a yellow powder 6C (350 mg, 90% yield).

Step 4 6C (150 mg, 0.35 mmol, 1 eq) was added to DCM (5 mL), before HATU (159 mg, 0.42 mmol, 1.2 eq) was added at room temperature. The system was stirred for 10 min, before NMM (89 mg, 0.873 mmol, 2 eq) and 6D (73 mg, 0.35 mmol, 1 eq) were added. The system was stirred overnight at room temperature. When TLC indicated the depletion of the starting materials, the reaction solution was poured into water (15 mL) and extracted with EA (30 mL×4). The organic phases were combined, concentrated at reduced pressure, and directly purified by thin-layer chromatography to give an off-white powder 6E (50 mg, 23% yield). MS-ESI: m/z 620.4 [M+H]⁺.

Step 5 6E (50 mg, 0.08 mmol, 1 eq) was dissolved in ethyl acetate (5 mL), before dioxane hydrochloride (4 N, 0.5 mL) was added in an ice-water bath. The system was stirred at room temperature overnight. When LCMS indicated the depletion of the starting materials, the mixture was concentrated at reduced pressure and directly purified by thin-layer chromatography to give a pale yellow powder 6 (24 mg, 70.8% yield).

MS-ESI: m/z 420.6 [M+H]⁺.

¹H NMR (400 MHz, Methanol-d₄) δ 7.66 (d, J=2.0 Hz, 1H), 7.55 (dd, J=8.1, 1.9 Hz, 1H), 7.43 (d, J=8.2 Hz, 1H), 7.41-7.36 (m, 2H), 6.89 (s, 1H), 6.78-6.71 (m, 2H), 4.58 (s, 2H), 3.47-3.38 (m, 4H), 1.76-1.60 (m, 4H), 1.07-0.77 (m, 6H).

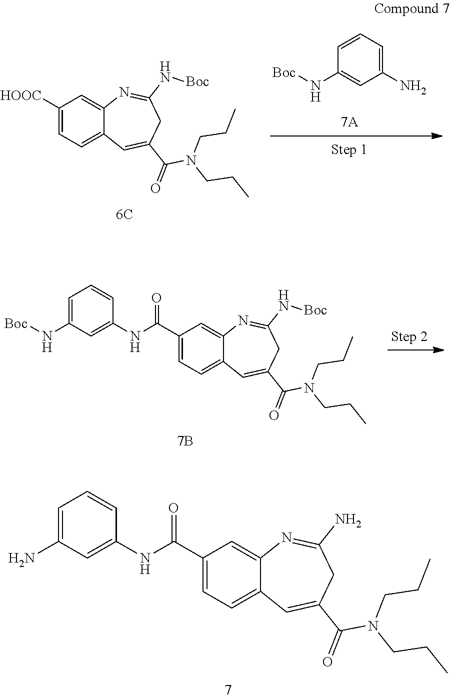 [SMILES: CCCN(CCC)C(=O)C1=Cc2ccc(C(=O)O)cc2N=C(NC(=O)OC(C)(C)C)C1 ; CC(C)(C)OC(=O)Nc1cccc(N)c1 ; CCCN(CCC)C(=O)C1=Cc2ccc(C(=O)Nc3cccc(NC(=O)OC(C)(C)C)c3)cc2N=C(NC(=O)OC(C)(C)C)C1 ; CCCN(CCC)C(=O)C1=Cc2ccc(C(=O)Nc3cccc(N)c3)cc2N=C(N)C1]

Step 1 6C (300 mg, 0.70 mmol) was added to a 100-mL flask, before DMF (10 mL), 7A (174 mg, 0.84 mmol), DIEA (271 mg, 2.10 mmol), and HATU (372 mg, 0.98 mmol) were added at 0° C. The reaction system was stirred at room temperature for 16 h until LCMS indicated the depletion of the starting materials. Water (100 mL) was added, and the mixture was extracted with EA (100 mL×3). The organic phase was concentrated by rotary evaporation, and purified by column chromatography (EA:PE=0 to 20%) to give a pale yellow oily liquid 7B (300 mg, 69% yield). MS-ESI: m/z 620.4 [M+H]⁺.

Step 2 7B (300 mg, 0.48 mmol) was added to a 100-mL flask, before DCM (8 mL) and TFA (2 mL) were added at 0° C. The reaction system was stirred at room temperature for 16 h until LCMS indicated the depletion of the starting materials. The reaction solution was concentrated by rotary evaporation and purified by reversed-phase column chromatography (NH₄HCO₃) to give a pale yellow solid 7 (70 mg, 34% yield).

MS-ESI: m/z 420.2 [M+H]⁺.

¹H NMR (400 MHz, DMSO-d₆) δ 9.90 (s, 1H), 7.60 (d, J=1.8 Hz, 1H), 7.43 (dd, J=8.1, 1.9 Hz, 1H), 7.38 (d, J=8.2 Hz, 1H), 7.14 (t, J=2.0 Hz, 1H), 6.94 (t, J=7.9 Hz, 1H), 6.91-6.81 (m, 3H), 6.77 (s, 1H), 6.33-6.26 (m, 1H), 5.04 (s, 2H), 3.31-3.26 (m, 4H), 2.71 (s, 2H), 1.64-1.49 (m, 4H), 0.83 (brs, 6H).

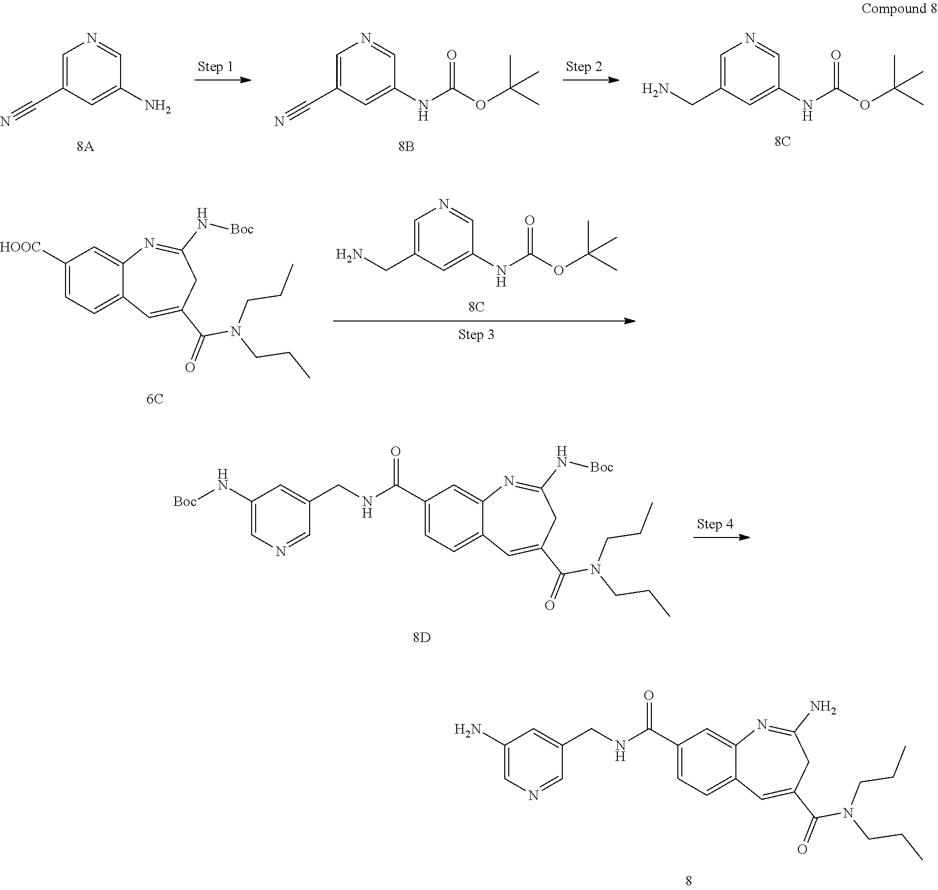

Step 1 8A (1 g, 8.4 mmol) was dissolved in DCM (20 mL), before DMAP (102 mg, 0.84 mmol) and Boc₂O (2.2 g, 10.1 mmol) were added. The system was stirred at room temperature overnight. After concentration, the mixture was purified by column chromatography (PE:EA=1:1) to give a white solid 8B (1.2 g, 65% yield, 95% purity). MS-ESI: m/z 220.0 [M+H]⁺.

Step 2 8B (500 mg, 2.27 mmol) was dissolved in MeOH/NH₃—H₂O (5 mL, v/v=4/1), before Raney Ni (100 mg) was added at room temperature. The system was purged three times with H₂ and stirred at room temperature overnight. The mixture was filtered and concentrated to give a green solid 8C (450 mg, 85% yield, 84% purity). MS-ESI: m/z 224.2 [M+H]⁺.

Step 1 6C (300 mg, 0.7 mmol) and 8C (156 mg, 0.7 mmol) were dissolved in DMF (3 mL), before DIEA (271 mg, 2.1 mmol) and HATU (320 mg, 0.84 mmol) were added. The system was stirred at room temperature overnight. The reaction solution was directly subjected to preparative HPLC (MeCN/H₂O) to give a pale yellow solid 8D (200 mg, 45% yield, 85% purity). MS-ESI: m/z 635.4 [M+H]⁺.

Step 4 8D (200 mg, 0.3 mmol) was dissolved in DCM (5 mL), before TFA (180 mg, 1.5 mmol) was added. The reaction system was stirred at room temperature for 3 h. The mixture was diluted with water and adjusted to pH 8-9 with NaHCO₃. The aqueous phase was extracted with ethyl acetate (100 mL×3). The organic phase was collected, washed with saturated brine (50 mL), dried over anhydrous sodium sulfate, filtered, concentrated by rotary evaporation, and subjected to preparative HPLC (MeCN/H₂O) to give a yellow solid 8 (52.8 mg, 35% yield, 95.23% purity).

MS-ESI: m/z 435.3 [M+H]⁺.

¹H NMR (400 MHz, DMSO-d₆) δ 7.90-7.77 (m, 2H), 7.64-7.58 (m, 1H), 7.52-7.44 (m, 1H), 7.40 (dd, J=8.1, 3.3 Hz, 1H), 7.10 (s, 1H), 6.90-6.84 (m, 1H), 4.55-4.47 (m, 2H), 3.47-3.36 (m, 4H), 3.34-3.32 (m, 2H), 1.78-1.56 (m, 4H), 1.11-0.71 (m, 6H).

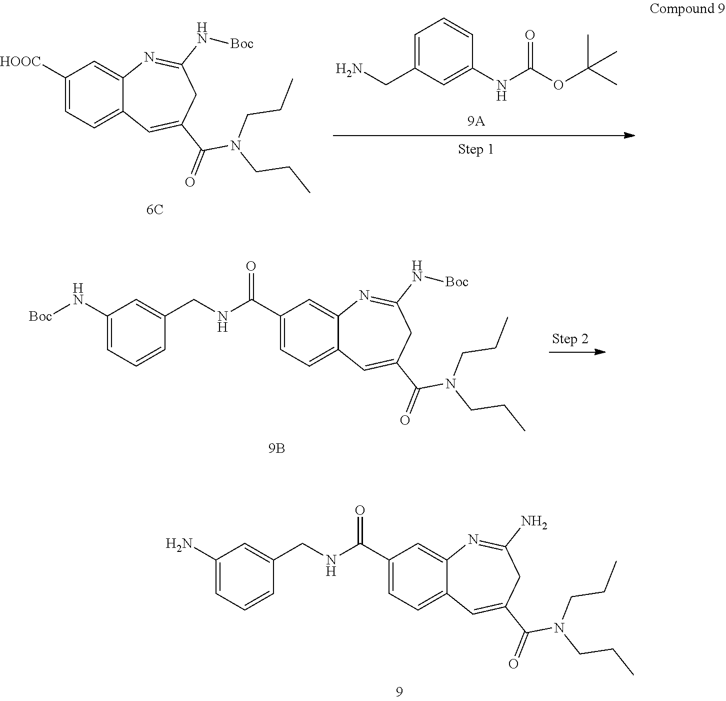 [SMILES: CCCN(CCC)C(=O)C1=Cc2ccc(C(=O)O)cc2N=C(NC(=O)OC(C)(C)C)C1 ; CC(C)(C)OC(=O)Nc1cccc(CN)c1 ; CCCN(CCC)C(=O)C1=Cc2ccc(C(=O)NCc3cccc(NC(=O)OC(C)(C)C)c3)cc2N=C(NC(=O)OC(C)(C)C)C1 ; CCCN(CCC)C(=O)C1=Cc2ccc(C(=O)NCc3cccc(N)c3)cc2N=C(N)C1]

Step 1 6C (300 mg, 0.7 mmol) was dissolved in DMF (3 mL), before 9A (155 mg, 0.7 mmol), DIEA (211 mg, 2.1 mmol) and HATU (320 mg, 0.84 mmol) were added. The system was stirred at room temperature overnight. The reaction solution was directly subjected to preparative HPLC (MeCN/H₂O) to give a white solid 9B (250 mg, 55% yield, 90% purity). MS-ESI: m/z 634.4[M+H]⁺.

Step 2 9B (250 mg, 0.4 mmol) was dissolved in DCM (5 mL), before TFA (135 mg, 1.2 mmol) was added. The reaction solution was incubated at room temperature for reaction for 3 h. The mixture was diluted with water and adjusted to pH 8-9 with NaHCO₃. The aqueous phase was extracted with ethyl acetate (100 mL×3). The organic phase was collected, washed with saturated brine (50 mL), dried over anhydrous sodium sulfate, filtered, concentrated by rotary evaporation, and subjected to preparative HPLC (MeCN/H₂O) to give a pale yellow solid 9 (26.3 mg, 15% yield, 99.59% purity).

MS-ESI: m/z 434.3 [M+H]⁺.

¹H NMR (400 MHz, Methanol-d₄) δ 7.61 (d, J=1.8 Hz, 1H), 7.48 (dd, J=8.1, 1.9 Hz, 1H), 7.39 (d, J=8.2 Hz, 1H), 7.06 (t, J=7.7 Hz, 1H), 6.87 (s, 1H), 6.74 (t, J=2.0 Hz, 1H), 6.71-6.66 (m, 1H), 6.62 (ddd, J=8.0, 2.4, 1.0 Hz, 1H), 4.49 (s, 2H), 3.46-3.37 (m, 4H), 1.76-1.57 (m, 4H), 1.11-0.72 (m, 6H).

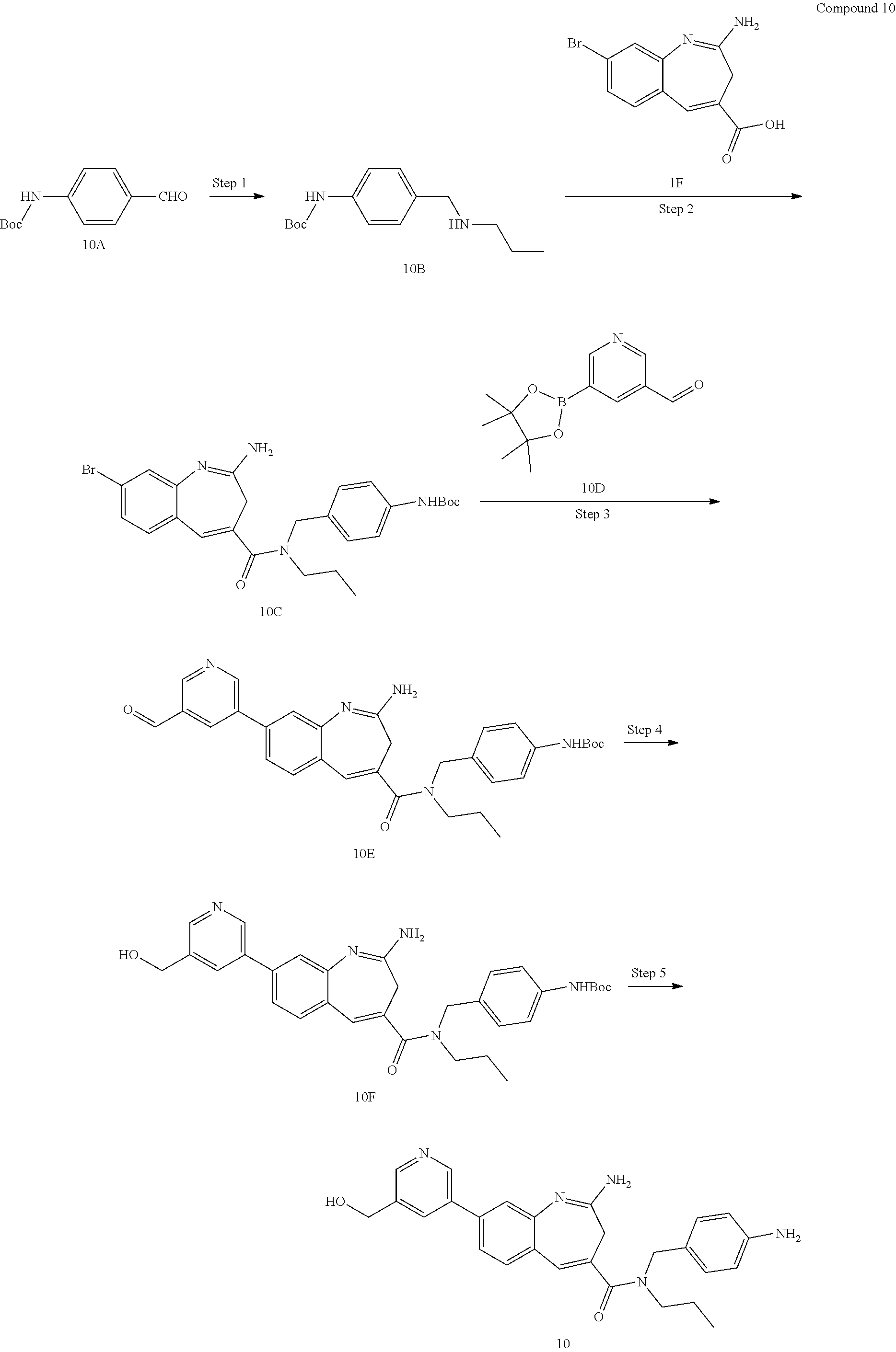

Step 1 10A (5 g, 22.60 mmol, 1 eq) was added to DCM (100 mL), before n-propylamine (4.01 g, 67.80 mmol, 3 eq) was added at room temperature. After the addition, the system was stirred at room temperature overnight. At room temperature, sodium cyanoborohydride (2.84 g, 45.20 mmol, 2 eq) was slowly added, and after the addition, the system was stirred at room temperature overnight. The reaction solution was poured into 100 mL of water and the phases were separated. The aqueous phase was extracted with DCM (100 mL×2). The organic phases were combined, dried over sodium sulfate, concentrated at reduced pressure, mixed with silica gel, and subjected to column chromatography (DCM:MeOH=50:1 to 10:1) to give a white solid 10B (3 g, 50.3% yield).

Step 2 1F (1 g, 3.15 mmol, 1 eq) was dissolved in DMF (20 mL). The temperature was reduced to around 0° C., before HBTU (1.43 g, 3.78 mmol, 1.2 eq) and DIEA (1.63 g, 12.60 mmol, 4 eq) were added. The reaction solution was stirred for 10 min at 0° C., before 10B (1 g, 3.78 mmol, 1.2 eq) was added. After the addition, the system was stirred at room temperature overnight. The reaction solution was poured into water (60 mL) and extracted with EA (50 mL×5). The organic phases were combined, washed with saturated brine (50 mL×2), dried over sodium sulfate, concentrated at reduced pressure, mixed with silica gel, and subjected to column chromatography (DCM:MeOH=20:1 to 10:1). The product was then concentrated at reduced pressure to give a yellow solid 10C (550 mg, 30% yield). MS-ESI: m/z 527.2 [M+H]⁺.

Step 3 10C (550 mg, 1.04 mmol, 1 eq) was added to a mixed solvent of dioxane (20 mL) and water (2 mL), before 10D (243 mg, 1.04 mmol, 1 eq) and anhydrous potassium phosphate (664 mg, 3.13 mmol, 3 eq) were added. The system was purged twice with argon before Pd(PPh₃)₄ (110 mg) was added, then purged twice again with argon, heated to 90° C., and incubated for reaction for 3 h. When LCMS indicated the depletion of the starting materials, the reaction solution was poured into 50 mL of water and extracted with EA (50 mL×3). The organic phases were combined, dried over sodium sulfate, concentrated at reduced pressure, and subjected to column chromatography (PE:EA=2:1 to 0:1) to give a white solid 10E (200 mg, 34.7% yield). MS-ESI: m/z 554.4 [M+H]⁺.

Step 4 10E (200 mg, 0.36 mmol, 1 eq) was added to methanol (10 mL), before sodium borohydride (41 mg, 1.08 mmol, 3 eq) was slowly added in portions at room temperature. After the addition, the system was incubated at room temperature for reaction for 2 h. When LCMS indicated the depletion of the starting materials, the reaction solution was quenched with saturated aqueous ammonium chloride (5 mL), and the mixture was concentrated at reduced pressure to remove the solvent and directly purified by thin-layer chromatography to give a white solid 10F (160 mg, 80% yield). MS-ESI: m/z 556.3 [M+H]⁺.

Step 5 10F (160 mg, 0.28 mmol, 1 eq) was dissolved in EA (5 mL), before 4 N dioxane hydrochloride (0.5 mL) was added in an ice-water bath. The system was stirred at room temperature overnight. When LCMS indicated the depletion of the starting materials, the mixture was concentrated at reduced pressure, subjected to preparative chromatography, and lyophilized to give a white powder 10 (30 mg, 23% yield).

MS-ESI: m/z 456.3 [M+H]⁺.

¹H NMR (400 MHz, Methanol-d₄) δ 8.88 (d, J=2.2 Hz, 1H), 8.66 (d, J=1.9 Hz, 1H), 8.34 (s, 1H), 7.84-7.56 (m, 3H), 7.56-7.18 (m, 4H), 7.16 (s, 1H), 4.81 (s, 2H), 4.78 (s, 2H), 3.55-3.45 (m, 2H), 3.45-3.33 (m, 2H), 1.76-1.63 (m, 2H), 0.92 (s, 3H).

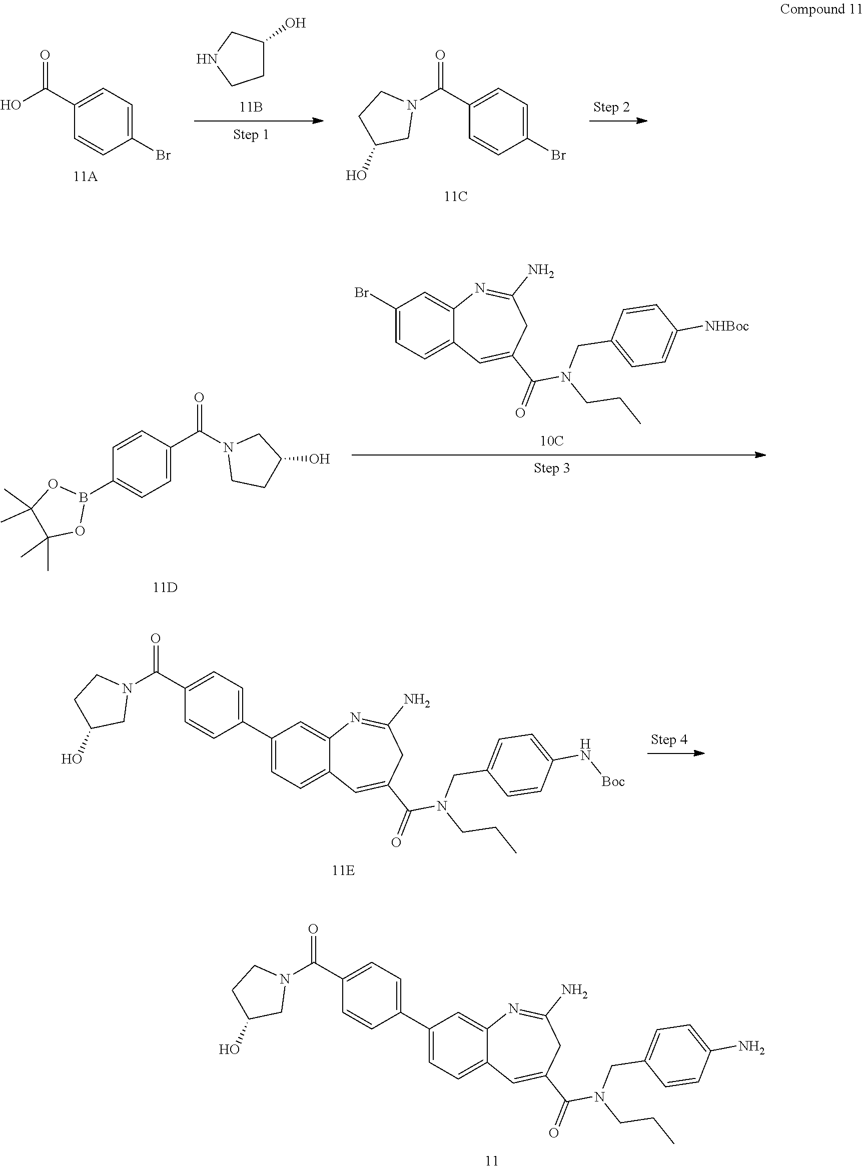

Step 1 11A (1 g, 4.97 mmol, 1 eq) was dissolved in DMF (20 mL), before DIEA (1.93 g, 14.92 mmol, 3 eq) and HATU (2.08 g, 5.47 mmol, 1.1 eq) were added at room temperature. After the addition, the system was stirred at room temperature for 5 min, and 11B (0.434 g, 4.97 mmol, 1 eq) was added. The system was stirred at room temperature overnight after the addition. The reaction solution was poured into 60 mL of water and extracted with EA (50 mL×3). The organic phase was dried over sodium sulfate, concentrated at reduced pressure, mixed with silica gel, and subjected to column chromatography (PE:EA=5:1) to give a white solid 11C (1 g, 74.6% yield). MS-ESI: m/z 270.0 [M+H]⁺.

Step 2 11C (0.9 g, 3.33 mmol, 1 eq) was dissolved in dioxane (20 mL), before bis(pinacolato)diboron (931 mg, 3.67 mmol, 1.1 eq) and AcOK (654 mg, 6.66 mmol, 2 eq) were added. The system was purged twice with argon before Pd(dppf)Cl₂ (0.2 g) was added in one portion, then purged twice again with argon, heated to 85° C., and incubated for reaction for 3 h. When LCMS indicated the depletion of the starting materials, the reaction solution was cooled to room temperature and directly loaded on a silica gel column (PE:EA=2:1) for purification to give a pale yellow solid 11D (300 mg, 28.3% yield). MS-ESI: m/z 318.3 [M+H]⁺.

Step 3 10C (300 mg, 0.569 mmol, 1 eq) and 11D (180.4 mg, 0.569 mmol, 1 eq) were added to a mixed solvent of dioxane (10 mL) and water (1 mL), before sodium carbonate (150.71 mg, 1.42 mmol, 2.5 eq) was added. The system was purged twice with argon before Pd(dppf)Cl₂ (50 mg) was added in one portion, then purged twice again with argon, heated to 85° C., and incubated for reaction for 3 h. When LCMS indicated the depletion of the starting materials, the reaction solution was cooled to room temperature, dried over sodium sulfate, directly mixed with silica gel, and purified by column chromatography to give a yellow solid 11E (100 mg, 27.6% yield). MS-ESI: m/z 638.4 [M+H]⁺.

Step 4 11E (100 mg, 0.157 mmol, 1 eq) was dissolved in EA (5 mL), before 4 N dioxane hydrochloride (1 mL) was added in an ice-water bath. The system was stirred at room temperature overnight. When LCMS indicated the depletion of the starting materials, the mixture was concentrated at reduced pressure, subjected to preparative chromatography, and lyophilized to give a yellow solid 11 (30 mg, 35.7% yield).

MS-ESI: m/z 538.3 [M+H]⁺.

¹H NMR (400 MHz, Methanol-d₄) δ 7.80 (d, J=7.9 Hz, 2H), 7.76-7.66 (m, 4H), 7.66-7.30 (m, 3H), 7.25 (s, 2H), 7.14 (s, 1H), 4.78 (s, 2H), 4.45 (d, J=47.8 Hz, 1H), 3.84-3.58 (m, 3H), 3.58-3.32 (m, 5H), 2.19-1.90 (m, 2H), 1.75-1.63 (m, 2H), 0.91 (s, 3H).

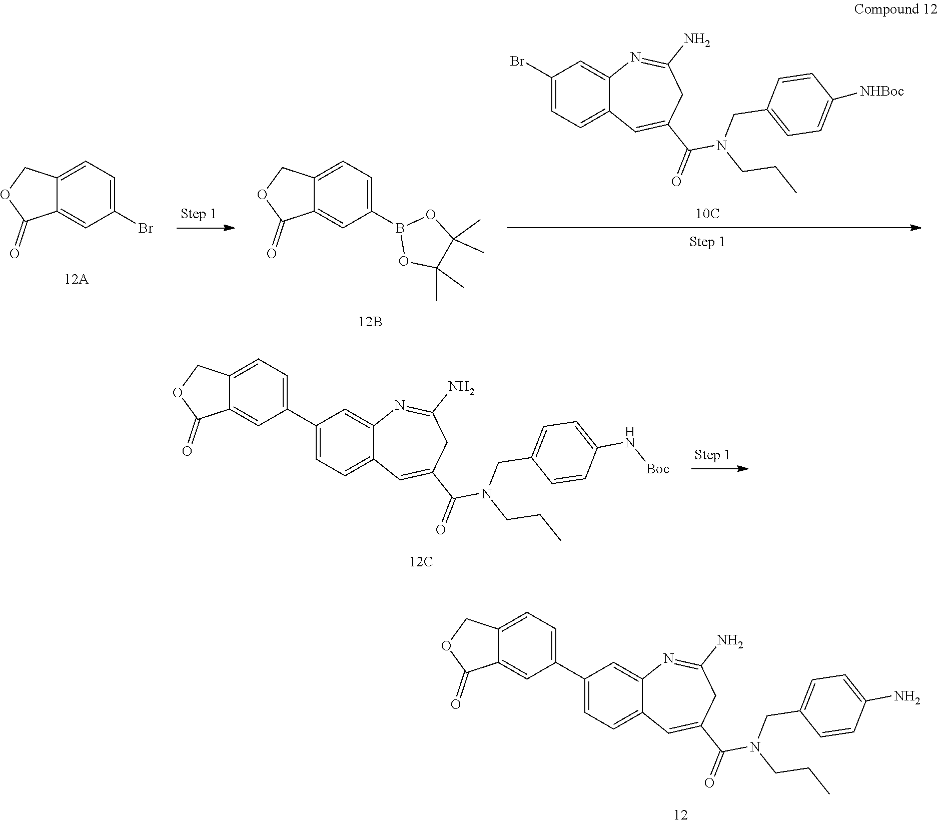 [SMILES: O=C1OCc2ccc(Br)cc21 ; CC1(C)OB(c2ccc3c(c2)C(=O)OC3)OC1(C)C ; CCCN(Cc1ccc(NC(=O)OC(C)(C)C)cc1)C(=O)C1=Cc2ccc(Br)cc2N=C(N)C1 ; CCCN(Cc1ccc(NC(=O)OC(C)(C)C)cc1)C(=O)C1=Cc2ccc(-c3ccc4c(c3)C(=O)OC4)cc2N=C(N)C1 ; CCCN(Cc1ccc(N)cc1)C(=O)C1=Cc2ccc(-c3ccc4c(c3)C(=O)OC4)cc2N=C(N)C1]

Step 1 12A (1 g, 4.69 mmol, 1 eq) was dissolved in dioxane (20 mL), before bis(pinacolato)diboron (1.31 g, 5.16 mmol, 1.1 eq) and AcOK (921.4 mg, 9.39 mmol, 2 eq) were added. The system was purged twice with argon before Pd(dppf)Cl₂ (0.1 g) was added in one portion, then purged twice again with argon, heated to 85° C., and incubated for reaction for 3 h. When LCMS indicated the depletion of the starting materials, the reaction solution was cooled to room temperature and directly loaded on a silica gel column (PE:EA=10:1) for purification to give a white solid 12B (300 mg, 24.6% yield).

MS-ESI: m/z 261.2 [M+H]⁺.

Step 2 10C (250 mg, 0.474 mmol, 1 eq) and 12B (247 mg, 0.948 mmol, 2 eq) were added to a mixed solvent of dioxane (10 mL) and water (1 mL), before potassium phosphate (302 mg, 1.42 mmol, 3 eq) was added. The system was purged twice with argon before Pd(dppf)Cl₂ (50 mg) was added in one portion, then purged twice again with argon, heated to 85° C., and incubated for reaction for 3 h. When LCMS indicated the depletion of the starting materials, the reaction solution was cooled to room temperature, dried over sodium sulfate, directly mixed with silica gel, and purified by column chromatography to give a yellow solid 12C (100 mg, 36.4% yield). MS-ESI: m/z 581.2 [M+H]⁺.

Step 3 12C (100 mg, 0.172 mmol, 1 eq) was dissolved in EA (5 mL), before 4 N dioxane hydrochloride (1 mL) was added in an ice-water bath. The system was stirred at room temperature overnight. When LCMS indicated the depletion of the starting materials, the mixture was concentrated at reduced pressure, subjected to preparative chromatography, and lyophilized to give a yellow solid 12 (23 mg, 28% yield).

MS-ESI: m/z 481.3 [M+H]⁺.

¹H NMR (400 MHz, Methanol-d₄) δ 8.17 (s, 1H), 8.10 (d, J=8.1 Hz, 1H), 7.84-7.70 (m, 3H), 7.70-7.27 (m, 3H), 7.27-7.06 (m, 3H), 5.47 (s, 2H), 4.77 (s, 2H), 3.54-3.44 (m, 2H), 3.44-3.32 (m, 2H), 1.77-1.64 (m, 2H), 0.92 (s, 3H).

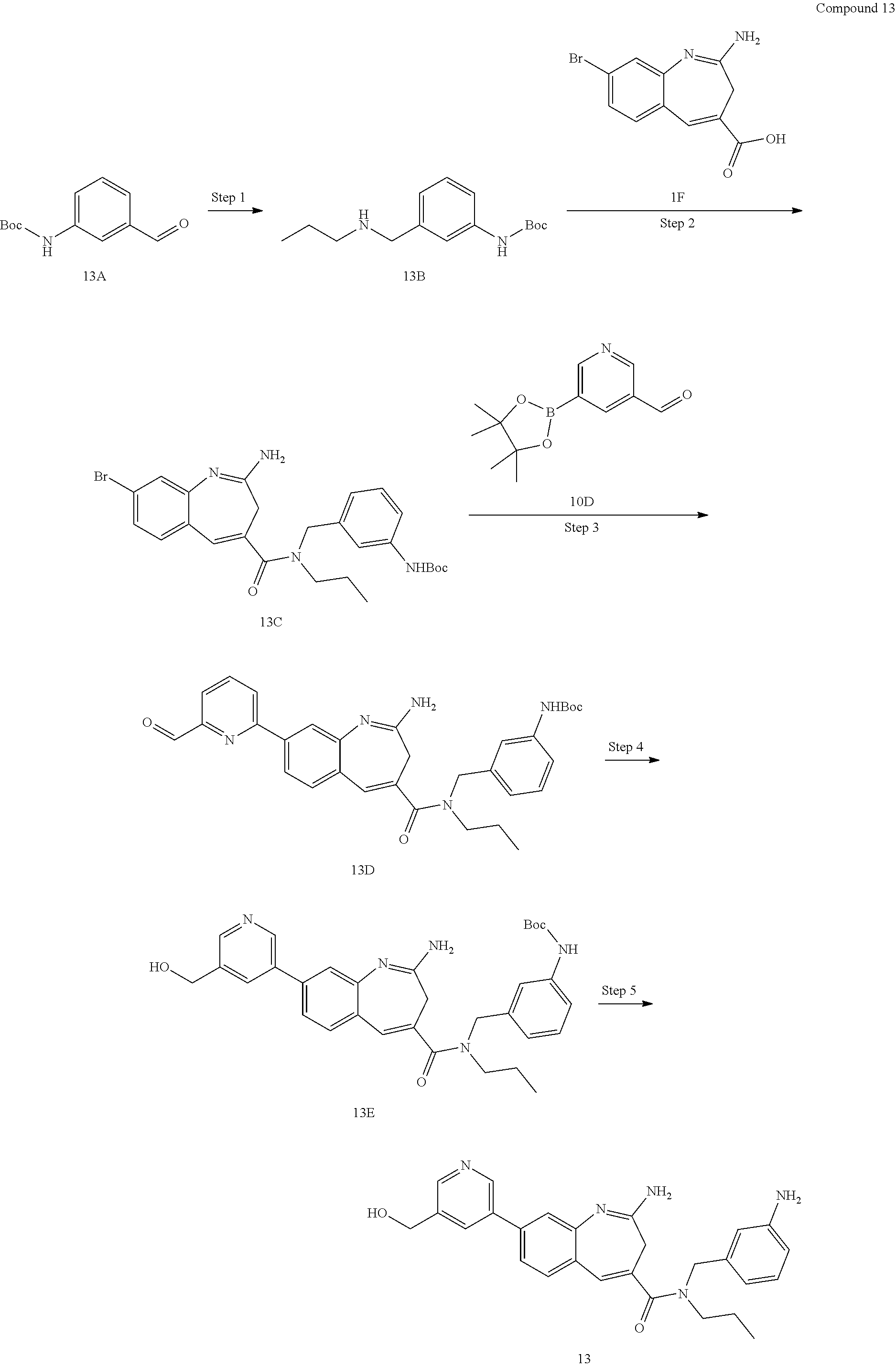

Step 1 13A (5 g, 22.60 mmol, 1 eq) was added to DCM (100 mL), before n-propylamine (4.01 g, 67.80 mmol, 3 eq) was added at room temperature. After the addition, the system was stirred at room temperature overnight. At room temperature, sodium cyanoborohydride (2.84 g, 45.20 mmol, 2 eq) was slowly added, and after the addition, the system was stirred at room temperature overnight. The reaction solution was poured into 100 mL of water and the phases were separated. The aqueous phase was extracted with DCM (100 mL×2). The organic phases were combined, dried over sodium sulfate, concentrated at reduced pressure, mixed with silica gel, and subjected to column chromatography (DCM:MeOH=50:1 to 10:1) to give a white solid 13B (1.5 g, 25.1% yield). MS-ESI: m/z 265.2 [M+H]⁺.

Step 2 1F (2 g, 6.30 mmol, 1 eq) was dissolved in DMF (40 mL). The temperature was reduced to around 0° C., before HBTU (2.86 g, 7.56 mmol, 1.2 eq) and DIEA (3.26 g, 25.20 mmol, 4 eq) were added. The reaction solution was stirred for 10 min at 0° C., before 13B (2 g, 7.56 mmol, 1.2 eq) was added. After the addition, the system was stirred at room temperature overnight. When LCMS indicated the depletion of the starting material 1F, the reaction solution was poured into water (120 mL) and extracted with EA (100 mL×5). The organic phases were combined, washed with saturated brine (100 mL×2), dried over sodium sulfate, concentrated at reduced pressure, mixed with silica gel, and subjected to column chromatography (DCM:MeOH=20:1 to 10:1). The product was then concentrated at reduced pressure to give a yellow solid 13C (1.2 g, 36.1% yield). MS-ESI: m/z 527.2 [M+H]⁺.

Step 3 13C (1.2 g, 2.28 mmol, 1 eq) was added to a mixed solvent of dioxane (40 mL) and water (4 mL), before 10D (530.3 mg, 2.28 mmol, 1 eq) and anhydrous potassium phosphate (1.45 g, 6.83 mmol, 3 eq) were added. The system was purged twice with argon before Pd(PPh₃)₄ (220 mg) was added, then purged twice again with argon, heated to 90° C., and incubated for reaction for 3 h. When LCMS indicated the depletion of the starting materials, the reaction solution was poured into 100 mL of water and extracted with EA (100 mL×3). The organic phases were combined, dried over sodium sulfate, concentrated at reduced pressure, and subjected to column chromatography (PE:EA=2:1 to 0:1) to give a white solid 13D (250 mg, 19.9% yield). MS-ESI: m/z 554.3 [M+H]⁺.

Step 4 13D (250 mg, 0.45 mmol, 1 eq) was added to methanol (10 mL), before sodium borohydride (51.3 mg, 1.35 mmol, 3 eq) was slowly added in portions at room temperature. After the addition, the system was incubated at room temperature for reaction for 2 h. The reaction solution was quenched with 5 mL of saturated aqueous ammonium chloride, and the mixture was concentrated at reduced pressure to remove the solvent and directly purified by thin-layer chromatography to give a white solid 13E (124 mg, 49.4% yield). MS-ESI: m/z 556.2 [M+H]⁺.

Step 5 13E (124 mg, 0.22 mmol, 1 eq) was dissolved in EA (5 mL), before 4 N dioxane hydrochloride (0.5 mL) was added in an ice-water bath. The system was stirred at room temperature overnight. When LCMS indicated the depletion of the starting materials, the mixture was concentrated at reduced pressure, adjusted to pH 8 with saturated NaHCO₃ solution, extracted with EA (10 mL×3), concentrated, mixed with silica gel, and subjected to column chromatography (DCM:MeOH=30:1 to 10:1) and lyophilized to give a white powder 13 (25 mg, 24.6% yield). MS-ESI: m/z 456.2 [M+H]⁺.

¹H NMR (400 MHz, DMSO-d₆) δ 8.76 (d, J=2.0 Hz, 1H), 8.50 (s, 1H), 7.99 (s, 1H), 7.32 (s, 1H), 7.28-7.26 (d, J=7.7 Hz, 1H), 6.99-6.97 (d, J=7.6 Hz, 1H), 6.86 (s, 2H), 6.47-6.45 (d, J=8.1 Hz, 3H), 5.08 (s, 2H), 4.61 (s, 2H), 4.48 (s, 2H), 3.32-3.21 (m, 2H), 2.79 (s, 2H), 1.56-1.52 (m, 2H), 1.23 (s, 1H), 0.85-0.81 (m, 3H).

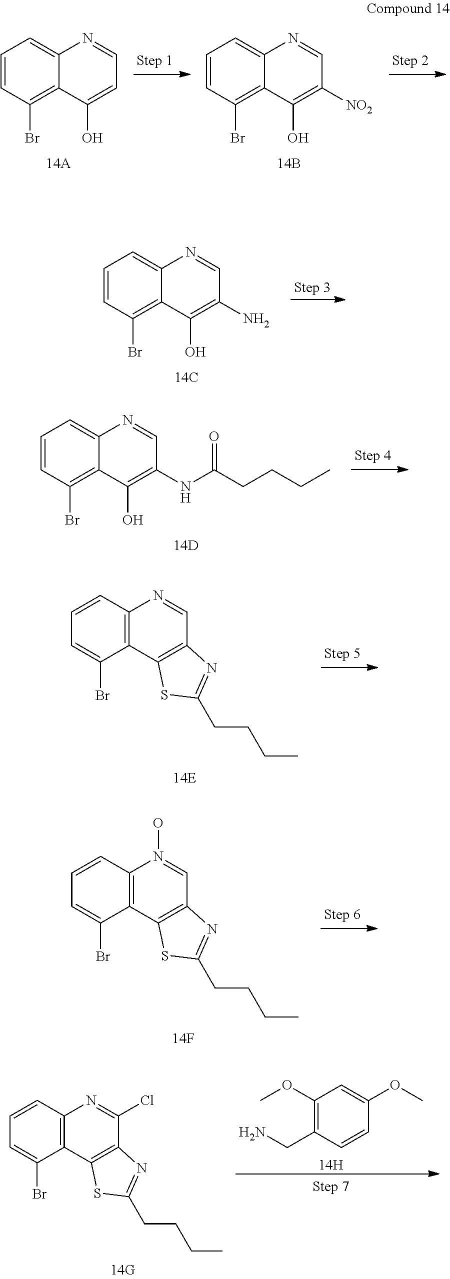

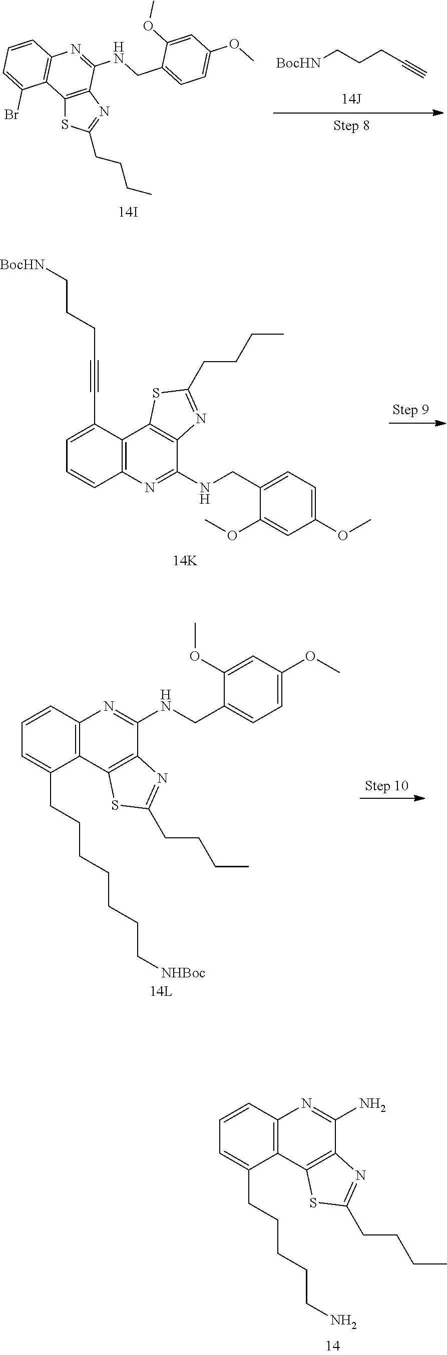 [SMILES: CCCCc1nc2c(NCc3ccc(OC)cc3OC)nc3cccc(Br)c3c2s1 ; C#CCCCNC(=O)OC(C)(C)C ; CCCCc1nc2c(NCc3ccc(OC)cc3OC)nc3cccc(C#CCCCNC(=O)OC(C)(C)C)c3c2s1 ; CCCCc1nc2c(NCc3ccc(OC)cc3OC)nc3cccc(CCCCCCCNC(=O)OC(C)(C)C)c3c2s1 ; CCCCc1nc2c(N)nc3cccc(CCCCCN)c3c2s1]

Step 1 At room temperature, 14A (4.5 g, 20.2 mmol) and propionic acid (100 mL) were added into a 250-mL three-neck flask. The reaction solution was heated to 125° C., and HNO₃ (3.2 g, 50.8 mmol) was dropwise added with the internal temperature controlled at 120-130° C. The reaction system was stirred at 125° C. for 1 h until LCMS indicated the completion of the reaction. The reaction solution was cooled to room temperature, slowly poured into ethanol (500 mL) and filtered. The filter cake was rinsed sequentially with ethanol (100 mL), water (100 mL), and ethanol (100 mL). The filter cake was collected and dried to give a yellow solid 14B (3 g, 55.6% yield). MS-ESI: m/z 269.0[M+H]⁺.

Step 2 At room temperature, 14B (3 g, 11.2 mmol) was added into a 250-mL single-neck flask and dissolved in EtOH (50 mL) and H₂O (10 mL), before NH₄Cl (1.8 g, 33.6 mmol) and iron powder (3.1 g, 56 mmol) were sequentially added. The reaction system was stirred at 80° C. for 2 h until LCMS indicated the completion of the reaction. The reaction solution was filtered before cooling. Ethyl acetate (200 mL) and water (100 mL) were added to the filtrate, and the organic phase was separated. The aqueous phase was extracted with ethyl acetate (100 mL×3), and the organic phase was collected, washed with saturated brine (100 mL), dried over anhydrous sodium sulfate, filtered, and concentrated by rotary evaporation. The crude product was purified by column chromatography (PE/EA=10:1 to 3:1) to give a yellow solid 14C (2 g, 75% yield). MS-ESI: m/z 241.0 [M+H]⁺.

Step 3 14C (2 g, 8.4 mmol), DCM (20 mL), and TEA (1.3 g, 12.6 mmol) were added into a 100-mL three-neck flask. n-Valeryl chloride (1.2 g, 10.1 mmol) was slowly added in an ice bath, and the reaction system was stirred for 1 h at 25° C. When LCMS indicated the completion of the reaction, the reaction solution was poured into water (20 mL) and extracted with EA (50 mL×3). The organic phase was collected, washed with saturated brine (20 mL), and dried over anhydrous sodium sulfate. After filtration and concentration, the crude product was purified by column chromatography (PE/EA=3/1) to isolate a yellow oily liquid 14D (1.5 g, 55.6% yield). MS-ESI: m/z 323.0 [M+H]⁺.

Step 4 At room temperature, 14D (1.5 g, 4.7 mmol) and pyridine (50 mL) were added into a 250-mL single-neck flask, before P₂S₅ (9 g, 47 mmol) was added. The reaction system was stirred at 120° C. overnight. The reaction solution was concentrated by rotary evaporation, poured into water (100 mL), and extracted with EA (3×100 mL). The organic phase was collected, washed with saturated brine (100 mL), and dried over anhydrous sodium sulfate. After filtration and concentration, the crude product was purified by column chromatography (PE/EA=10/1) to give a yellow solid 14E (1 g, 66.7% yield). MS-ESI: m/z 321.0 [M+H]⁺.

Step 5 At room temperature, 14E (2.6 g, 8.1 mmol) and CHCl₃ (50 mL) were added into a 250-mL single-neck flask, before m-CPBA (3.3 g, 16.2 mmol) was slowly added in an ice bath. The reaction system was stirred at 25° C. overnight. The reaction solution was poured into DCM (200 mL). The mixture was sequentially washed with 5% sodium thiosulfate (50 mL), saturated sodium bicarbonate (50 mL), and saturated brine (50 mL), and dried over anhydrous sodium sulfate. After filtration and concentration, the crude product was purified by column chromatography (PE/EA=3/1) to give a yellow oily liquid 14F (2 g, 73.5% yield). MS-ESI: m/z 337.0 [M+H]⁺.

Step 6 14F (2 g, 5.95 mmol) was added into a 100-mL three-neck flask, before POCl₃ (27 g, 178.5 mmol) was slowly added in an ice bath. DIEA (2.3 g, 17.85 mmol) was slowly and dropwise added with the internal temperature kept at 15° C., and the reaction system was stirred at 100° C. overnight. When LCMS indicated the completion of the reaction, the reaction solution was cooled to room temperature, concentrated by rotary evaporation at reduced pressure to remove the solvent, and poured slowly into ice water (100 mL). The mixture was adjusted to pH 9 with solid potassium carbonate and the aqueous phase was extracted with ethyl acetate (100 mL×3). The organic phase was collected, washed with saturated brine (50 mL), and dried over anhydrous sodium sulfate. After filtration and concentration by rotary evaporation, the residue was purified by column chromatography (PE/EA=10/1) to give a yellow oily liquid 14G (1.8 g, 85.7% yield). MS-ESI: m/z 355.0 [M+H]⁺.

Step 7 At room temperature, 14G (1.8 g, 5.1 mmol) and toluene (20 mL) were added into a 100-mL single-neck flask, before 14H (8.5 g, 51 mmol) and 2-chlorobenzoic acid (799 mg, 5.1 mmol) were sequentially added at room temperature. The reaction system was stirred at 120° C. overnight. When LCMS indicated the completion of the reaction, the reaction solution was concentrated by rotary evaporation at reduced pressure to remove the solvent. 100 mL of water was added, and the mixture was extracted with EA (3×100 mL). The organic phase was collected, washed with saturated brine (50 mL), and dried over anhydrous sodium sulfate. After filtration and concentration, the crude product was purified by reversed-phase column chromatography (neutral separation system) to give a yellow solid 14I (2 g, 80% yield). MS-ESI: m/z 486.1 [M+H]⁺.

Step 8 At room temperature, 141 (0.5 g, 1.03 mmol) and DMF (20 mL) were added into a 100-mL single-neck flask, before 14J (377 mg, 2.06 mmol), Pd(PPh₃)₂Cl₂ (75 mg, 0.103 mmol) and Cu₂O (442 mg, 3.09 mmol) were sequentially added at room temperature. The reaction system was stirred overnight at 110° C. in an N₂ atmosphere. When LCMS indicated the completion of the reaction, 100 mL of water was added to the reaction solution, and the mixture was extracted with EA (100 mL×3). The organic phase was collected, washed with saturated brine (50 mL), and dried over anhydrous sodium sulfate. After filtration and concentration, the crude product was separated by column chromatography (PE:EA=3:1) to give a yellow oily liquid 14K (0.3 g, 49.5% yield). MS-ESI: m/z 589.3 [M+H]⁺.

Step 9 At room temperature, 14K (0.3 g, 0.51 mmol) and THE (20 mL) were added into a 100-mL single-neck flask, before Pd/C (50 mg) was added at room temperature. The system was purged 3 times with H₂ and stirred at 25° C. overnight. When LCMS indicated the completion of the reaction, the reaction solution was filtered through celite and rinsed with THE (3×20 mL). The filtrate was concentrated by rotary evaporation at reduced pressure, and the crude product was separated by column chromatography (PE/EA=5/1) to give a yellow oily liquid 14 L (0.2 g, 66.7% yield). MS-ESI: m/z 593.3 [M+H]⁺.

Step 10 At room temperature, 14 L (200 mg, 0.34 mmol) was added into a 100-mL single-neck flask, before TFA (5 mL) was slowly added in an ice bath. The reaction system was stirred at 80° C. for 15 min. When LCMS indicated the completion of the reaction, the reaction solution was concentrated by rotary evaporation at reduced pressure to remove the solvent and purified by reversed-phase column chromatography (neutral separation system) to give a white solid 14 (77.6 mg, 66.9% yield). MS-ESI: m/z 343.2 [M+H]⁺.

¹H NMR (400 MHz, Methanol-d₄) δ 7.57 (d, J=8.3 Hz, 1H), 7.49-7.42 (m, 1H), 7.18 (d, J=7.2 Hz, 1H), 3.23-3.15 (m, 2H), 3.11-3.02 (m, 2H), 2.72 (t, J=6.7 Hz, 2H), 1.97-1.85 (m, 2H), 1.76-1.65 (m, 2H), 1.64-1.46 (m, 6H), 1.02 (t, J=7.4 Hz, 3H).

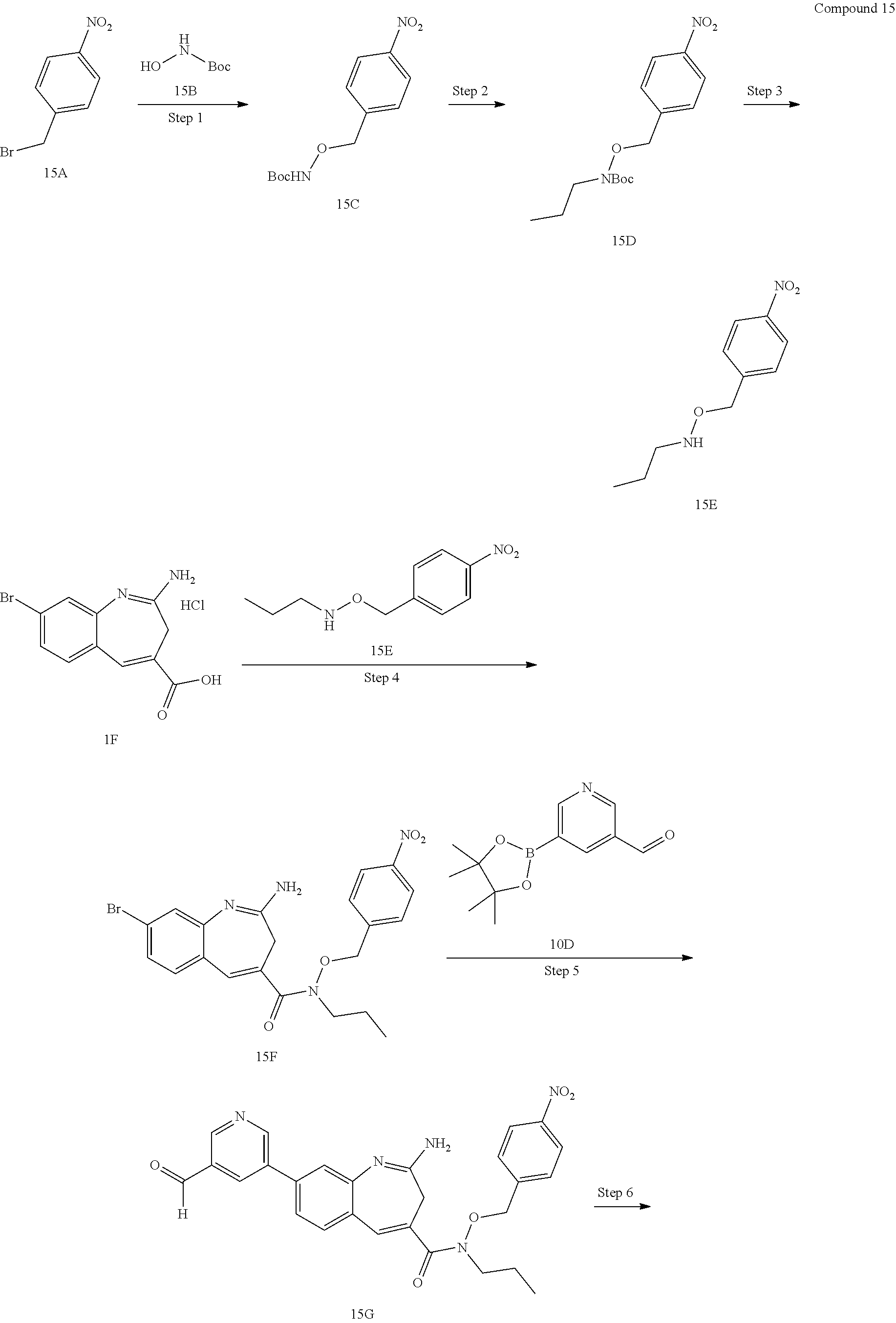

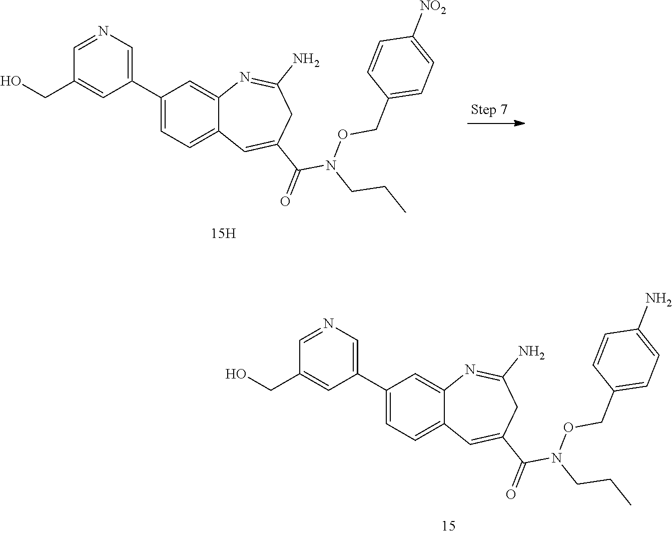 [SMILES: CCCN(OCc1ccc([N+](=O)[O-])cc1)C(=O)C1=Cc2ccc(-c3cncc(CO)c3)cc2N=C(N)C1 ; CCCN(OCc1ccc(N)cc1)C(=O)C1=Cc2ccc(-c3cncc(CO)c3)cc2N=C(N)C1]

Step 1 15A (5 g, 23.14 mmol, 1 eq) was dissolved in acetonitrile (100 mL), before tert-butyl N-hydroxycarbamate 15B (4.01 g, 30.09 mmol, 1.3 eq) was added at 0° C. DBU (3.52 g, 23.14 mmol, 1 eq) was then slowly and dropwise added, and after the addition, the system was warmed to room temperature and stirred for 3 h. When LCMS indicated the depletion of 15A, the reaction solution was concentrated at reduced pressure, before saturated potassium carbonate solution (100 mL) was added. The mixture was extracted with DCM (50 mL×3). The organic phases were combined, washed with saturated brine (50 mL), dried over sodium sulfate, concentrated at reduced pressure, mixed with silica gel, and subjected to column chromatography (PE:EA=5:1) to give a white solid 15C (4.5 g, 72.5% yield). MS-ESI: m/z 291.0 [M+Na]⁺.

Step 2 15C (4.5 g, 16.77 mmol, 1 eq) was dissolved in DMF (50 mL), and the system was cooled to 0° C. NaH (671 mg, 16.77 mmol, 1 eq, 60%) was added in one portion, and the mixture was stirred at 0° C. for 10 min before iodopropane (3.14 g, 18.45 mmol, 1.1 eq) was dropwise added. After the addition, the system was warmed to room temperature and incubated for reaction overnight. When LCMS indicated the depletion of the starting material 15C, the reaction solution was poured into ice water (150 mL) and extracted with EA (100 mL×3). The organic phases were combined, sequentially washed with 10% aqueous citric acid (100 mL), saturated sodium bicarbonate (100 mL) and saturated brine (100 mL), dried over sodium sulfate, concentrated at reduced pressure, mixed with silica gel, and subjected to column chromatography (PE:EA=5:1) to give a yellow solid 15D (4.5 g, 86.4% yield). MS-ESI: m/z 333.2 [M+Na]⁺.

Step 3 15D (4.5 g, 14.52 mmol, 1 eq) was dissolved in EA (50 mL), before 4 N dioxane hydrochloride (50 mL) was added in an ice-water bath. The system was stirred at room temperature overnight. When LCMS indicated the depletion of 15D, the mixture was directly filtered. The filter cake was rinsed with EA (100 mL), quickly transferred to a flask, and dried in vacuo to give a yellow solid 15E (2.5 g, 83.3% yield). MS-ESI: m/z 211.2 [M+H]⁺.

Step 4 1F (3 g, 9.45 mmol, 1 eq) and 15E (2.5 g, 10.39 mmol, 1.1 eq) were added to a mixed solvent of DMA (30 mL) and DCM (30 mL). The system was purged twice with argon before EDCI (7.24 g, 37.79 mmol, 4 eq) was added in one portion, then purged twice again with argon, warmed to room temperature, and incubated for reaction overnight. When LCMS indicated the depletion of the starting materials, the reaction solution was poured into 100 mL of water and extracted with DCM (100 mL×3). The organic phases were combined, washed with saturated brine (100 mL), dried over sodium sulfate, concentrated by rotary evaporation, mixed with silica gel, and subjected to column chromatography to give a yellow solid 15F (1.9 g, 42.5% yield). MS-ESI: m/z 473.1 [M+H]⁺.

Step 5 15F (1.9 g, 4.01 mmol, 1 eq) was added to a mixed solvent of dioxane (50 mL) and water (5 mL), before 10D (936 mg, 4.01 mmol, 1 eq) and anhydrous potassium phosphate (2.56 g, 12.04 mmol, 3 eq) were added. The system was purged twice with argon before Pd(PPh₃)₄ (200 mg) was added, then purged twice again with argon, heated to 85° C., and incubated for reaction for 3 h. When LCMS indicated the depletion of the starting materials, the reaction solution was poured into water (150 mL) and extracted with EA (150 mL×3). The organic phases were combined, dried over sodium sulfate, concentrated at reduced pressure, and subjected to column chromatography (DCM:MeOH=20:1) to give a dark red solid 15G (900 mg, 45% yield). MS-ESI: m/z 500.2 [M+H]⁺.

Step 6 15G (700 mg, 1.4 mmol, 1 eq) was added to methanol (20 mL), before sodium borohydride (32 mg, 0.84 mmol, 0.6 eq) was slowly added in portions at room temperature. After the addition, the system was incubated at room temperature for reaction for 2 h. The reaction was quenched with saturated aqueous ammonium chloride (50 mL), and the reaction solution was extracted with DCM (50 mL×3). The organic phases were combined, dried over sodium sulfate, concentrated at reduced pressure, and subjected to column chromatography (DCM:MeOH=20:1) to give a pale yellow solid 15H (200 mg, 22.2% yield). MS-ESI: m/z 502.2 [M+H]⁺.

Step 7 15H (200 mg, 0.31 mmol, 1 eq) was dissolved in methanol (10 mL), before Raney Ni (0.1 g) was added. The reaction system was purged twice with hydrogen and stirred for 3 h at room temperature in a hydrogen atmosphere. When LCMS indicated the depletion of 15H, the reaction solution was filtered through celite, concentrated by rotary evaporation, triturated in petroleum ether (10 mL) and methanol (2 mL), and filtered to give a yellow solid 15 (28 mg, 14.9% yield). MS-ESI: m/z 472.2 [M+H]⁺.

¹H NMR (400 MHz, DMSO-d₆) δ 8.80 (d, J=2.2 Hz, 1H), 8.52 (d, J=2.0 Hz, 1H), 8.02 (s, 1H), 7.41 (d, J=8.2 Hz, 1H), 7.36 (d, J=1.9 Hz, 1H), 7.29 (dd, J=8.0, 2.0 Hz, 1H), 7.09 (s, 1H), 6.89 (d, J=8.1 Hz, 2H), 6.83 (s, 2H), 6.41 (d, J=8.2 Hz, 2H), 5.37 (t, J=5.8 Hz, 1H), 5.16 (s, 2H), 4.69-4.52 (m, 4H), 3.63 (t, J=7.0 Hz, 2H), 2.77 (s, 2H), 1.71-1.58 (m, 2H), 0.90 (t, J=7.4 Hz, 3H).

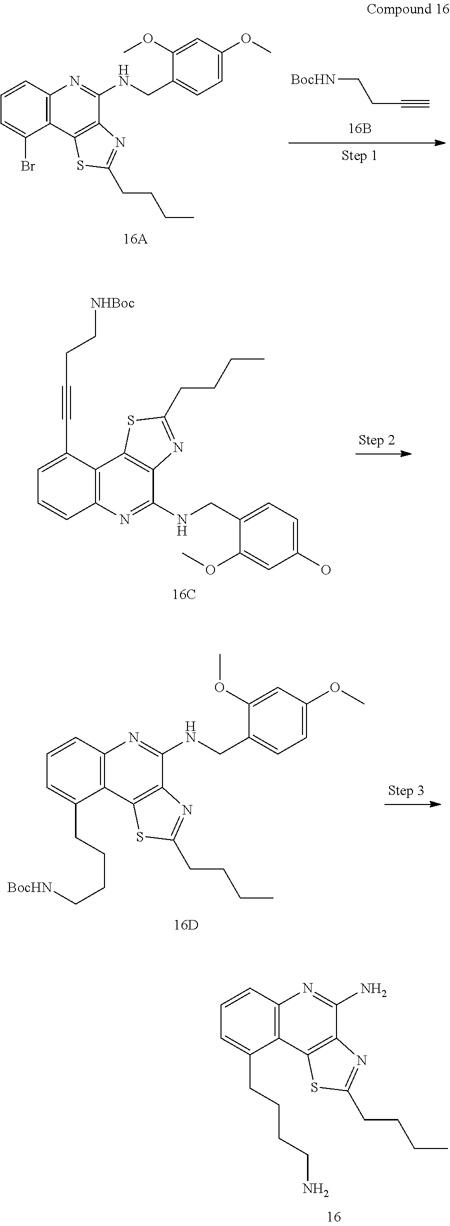 [SMILES: CCCCc1nc2c(NCc3ccc(OC)cc3OC)nc3cccc(Br)c3c2s1 ; C#CCCNC(=O)OC(C)(C)C ; CCCCc1nc2c(NCc3ccc(O)cc3OC)nc3cccc(C#CCCNC(=O)OC(C)(C)C)c3c2s1 ; CCCCc1nc2c(NCc3ccc(OC)cc3OC)nc3cccc(CCCCNC(=O)OC(C)(C)C)c3c2s1 ; CCCCc1nc2c(N)nc3cccc(CCCCN)c3c2s1]

Step 1 At room temperature, 16A (0.5 g, 1.03 mmol) and DMF (20 mL) were added into a 100-mL single-neck flask, before 16B (348 mg, 2.06 mmol), Pd(PPh₃)₂Cl₂ (75 mg, 0.103 mmol) and Cu₂O (442 mg, 3.09 mmol) were sequentially added at room temperature. The system was stirred at 110° C. in an N₂ atmosphere overnight until LCMS indicated the completion of the reaction. 100 mL of water was added to the reaction solution, and the mixture was extracted with EA (3×100 mL). The organic phase was collected, washed with saturated brine (50 mL), dried over anhydrous sodium sulfate, filtered, and concentrated. The crude product was separated by column chromatography (PE/EA=3/1) to give a yellow oily liquid 16C (0.3 g, 50.8% yield). MS-ESI: m/z 575.3 [M+H]⁺.

Step 2 At room temperature, 16C (0.3 g, 0.52 mmol) and THF (20 mL) were added into a 100-mL single-neck flask, before Pd/C (50 mg) was added at room temperature. The system was purged 3 times with H₂ and stirred overnight at 25° C. until LCMS indicated the completion of the reaction. The reaction solution was filtered through celite and rinsed with THF (3×20 mL). The filtrate was concentrated by rotary evaporation at reduced pressure, and the crude product was separated by column chromatography (PE/EA=5/1) to give a yellow oily liquid 16D (0.2 g, 66.2% yield). MS-ESI: m/z 579.4 [M+H]⁺.

Step 3 At room temperature, 16D (200 mg, 0.35 mmol) was added into a 100-mL single-neck flask, before TFA (5 mL) was slowly added in an ice bath. The system was stirred at 80° C. for 15 min until LCMS indicated the completion of the reaction. The reaction solution was concentrated by rotary evaporation at reduced pressure to remove the solvent and purified by reversed-phase column chromatography (neutral separation system) to give a white solid 16 (59.4 mg, 52.6% yield). MS-ESI: m/z 329.2[M+H]⁺.

¹H NMR (400 MHz, Methanol-d₄) δ 7.58 (dd, J=8.4, 1.3 Hz, 1H), 7.47 (dd, J=8.4, 7.2 Hz, 1H), 7.22 (dd, J=7.3, 1.3 Hz, 1H), 3.24-3.17 (m, 2H), 3.17-3.10 (m, 2H), 2.88-2.81 (m, 2H), 1.97-1.87 (m, 2H), 1.83-1.67 (m, 4H), 1.58-1.46 (m, 2H), 1.02 (t, J=7.4 Hz, 3H).

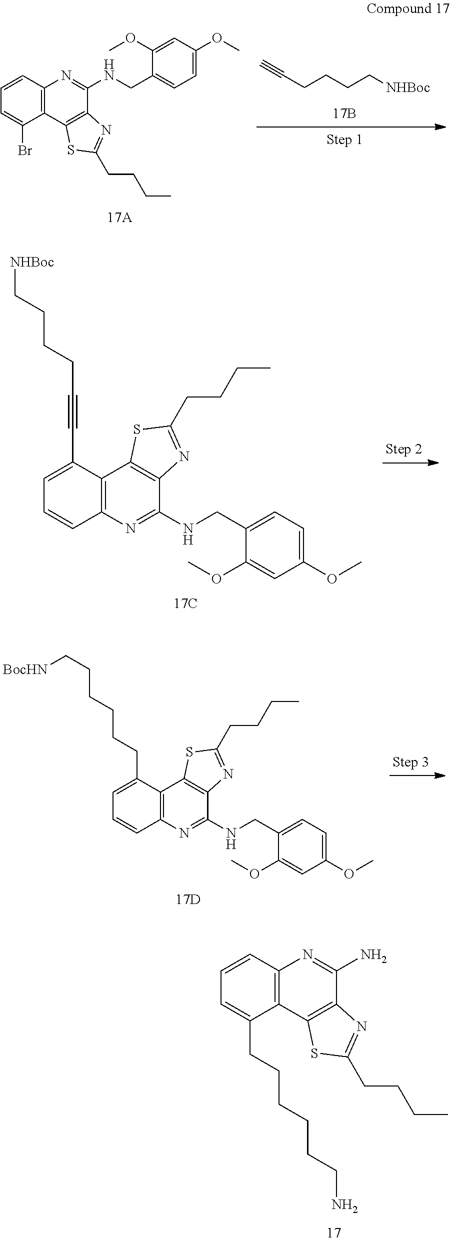

Step 1 At room temperature, 17A (0.5 g, 1.03 mmol) and DMF (20 mL) were added into a 100-mL single-neck flask, before 17B (203 mg, 2.06 mmol), Pd(PPh₃)₂Cl₂ (75 mg, 0.103 mmol) and Cu₂O (442 mg, 3.09 mmol) were sequentially added at room temperature. The system was stirred at 110° C. in an N₂ atmosphere overnight until LCMS indicated the completion of the reaction. 100 mL of water was added to the reaction solution, and the mixture was extracted with EA (3×100 mL). The organic phase was collected, washed with saturated brine (50 mL), dried over anhydrous sodium sulfate, filtered, and concentrated. The crude product was separated by column chromatography (PE/EA=3/1) to give a yellow oily liquid 17C (0.3 g, 48.4% yield). MS-ESI: m/z 603.4 [M+H]⁺.

Step 2 At room temperature, 17C (0.3 g, 0.50 mmol) and THF (20 mL) were added into a 100-mL single-neck flask, before Pd/C (50 mg) was added at room temperature. The system was purged 3 times with H₂ and stirred overnight at 25° C. until LCMS indicated the completion of the reaction. The reaction solution was filtered through celite and rinsed with THF (3×20 mL). The filtrate was concentrated by rotary evaporation at reduced pressure, and the crude product was separated by column chromatography (PE/EA=5/1) to give a yellow oily liquid 17D (0.2 g, 66.2% yield). MS-ESI: m/z 607.4 [M+H]⁺.

Step 3 At room temperature, 17D (200 mg, 0.33 mmol) was added into a 100-mL single-neck flask, before TFA (5 mL) was slowly added in an ice bath. The system was stirred at 80° C. for 15 min until LCMS indicated the completion of the reaction. The reaction solution was concentrated by rotary evaporation at reduced pressure and purified by reversed-phase column chromatography (neutral separation system) to give a white solid 17 (51.4 mg, 43.7% yield). MS-ESI: m/z 357.2[M+H]⁺.

¹H NMR (400 MHz, Methanol-d₄) δ 7.58 (dd, J=8.3, 1.3 Hz, 1H), 7.46 (dd, J=8.4, 7.2 Hz, 1H), 7.20 (dd, J=7.3, 1.3 Hz, 1H), 3.25-3.18 (m, 2H), 3.13-3.07 (m, 2H), 2.78-2.71 (m, 2H), 1.98-1.87 (m, 2H), 1.78-1.67 (m, 2H), 1.62-1.39 (m, 8H), 1.02 (t, J=7.4 Hz, 3H).

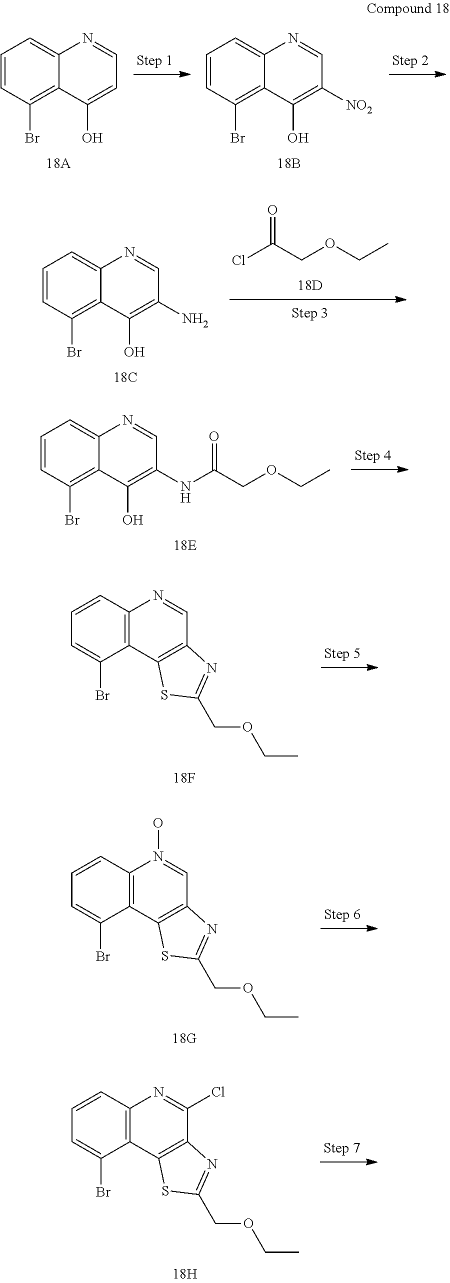

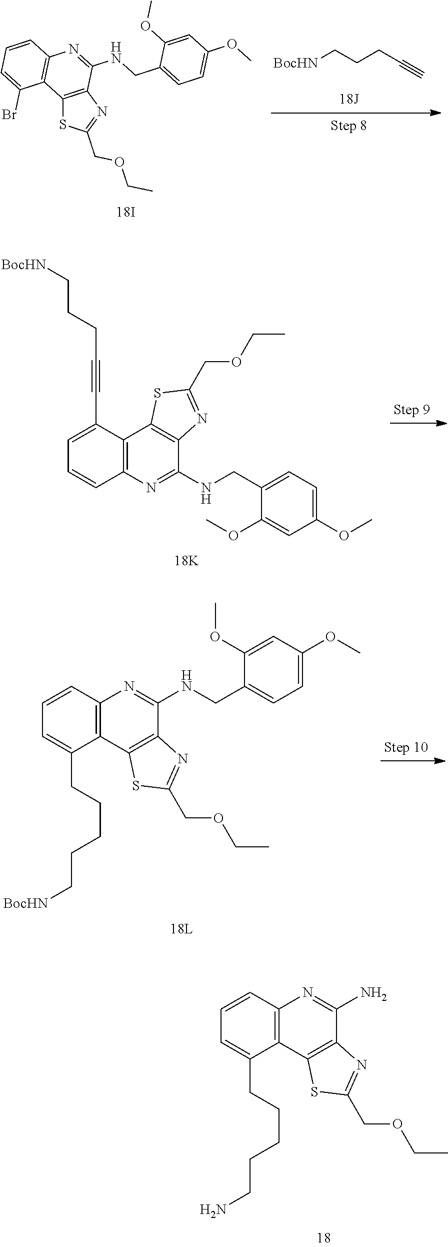 [SMILES: CCOCc1nc2c(NCc3ccc(OC)cc3OC)nc3cccc(Br)c3c2s1 ; C#CCCCNC(=O)OC(C)(C)C ; CCOCc1nc2c(NCc3ccc(OC)cc3OC)nc3cccc(C#CCCCNC(=O)OC(C)(C)C)c3c2s1 ; CCOCc1nc2c(NCc3ccc(OC)cc3OC)nc3cccc(CCCCCNC(=O)OC(C)(C)C)c3c2s1 ; CCOCc1nc2c(N)nc3cccc(CCCCCN)c3c2s1]

Step 1 At room temperature, 18A (10 g, 44.8 mmol) and propionic acid (200 mL) were added into a 250-mL three-neck flask. The reaction solution was heated to 125° C., and HNO₃ (8.6 g, 136.6 mmol) was dropwise added with the internal temperature controlled at 120-130° C. The reaction system was stirred at 125° C. for 1 h until LCMS indicated the completion of the reaction. The reaction solution was cooled to room temperature, slowly poured into ethanol (200 mL) and filtered. The filter cake was rinsed sequentially with ethanol (100 mL), water (100 mL), and ethanol (100 mL). The filter cake was collected and dried to give a yellow solid 18B (7 g, 58% yield). MS-ESI: m/z 269.0[M+H]⁺.

Step 2 At room temperature, 18B (7 g, 26.1 mmol) was added into a 250-mL single-neck flask and dissolved in EtOH (100 mL) and H₂O (20 mL), before NH₄Cl (4.23 g, 78.3 mmol) and Fe powder (7.31 g, 130.5 mmol) were sequentially added. The reaction system was stirred at 80° C. for 2 h until LCMS indicated the completion of the reaction. The reaction solution was filtered before cooling. Ethyl acetate (200 mL) and water (100 mL) were added to the filtrate, and the organic phase was separated. The aqueous phase was extracted with ethyl acetate (3×100 mL), and the organic phase was collected, washed with saturated brine (100 mL), dried over anhydrous sodium sulfate, filtered, and concentrated by rotary evaporation. The crude product was purified by column chromatography (DCM/MeOH=10:1) to give a yellow solid 18C (4.5 g, 72% yield). MS-ESI: m/z 239.0[M+H]⁺.

Step 3 18C (2.2 g, 9.2 mmol) and DCM (100 mL) were added into a 250-mL three-neck flask, before TEA (1.4 g, 13.8 mmol) was added. 18D (1.35 g, 11.04 mmol) was slowly added in an ice bath and the reaction system was stirred for 1 h at 25° C. until LCMS indicated the completion of the reaction. The reaction solution was poured into water (20 mL) and extracted with EA (3×50 mL). The organic phase was collected, washed with saturated brine (20 mL), dried over anhydrous sodium sulfate, filtered, and concentrated. The crude product was separated by column chromatography (PE/EA=3/1) to give a yellow solid 18E (1.6 g, 53.5% yield). MS-ESI: m/z 325.0[M+H]⁺.

Step 4 At room temperature, 18E (2.8 g, 8.6 mmol) and pyridine (120 mL) were added into a 250-mL single-neck flask, before P₂S₅ (16.4 g, 86 mmol) was added. The reaction system was stirred at 120° C. overnight until LCMS indicated the completion of the reaction. The reaction solution was concentrated by rotary evaporation to remove the solvent, poured into water (100 mL), and extracted with EA (3×100 mL). The organic phase was collected, washed with saturated brine (100 mL), dried over anhydrous sodium sulfate, filtered, and concentrated. The crude product was separated by chromatography (PE/EA=10/1) to give a yellow solid 18F (0.5 g, 18.5% yield). MS-ESI: m/z 323.0[M+H]⁺.

Step 5 At room temperature, 18F (0.5 g, 1.55 mmol) and CHCl₃ (10 mL) were added into a 250-mL single-neck flask, before m-CPBA (0.53 g, 3.1 mmol) was slowly added in an ice bath. The reaction system was stirred at 25° C. for 2 h until LCMS indicated the completion of the reaction. The reaction solution was poured into DCM (50 mL). The mixture was sequentially washed with 5% sodium thiosulfate (50 mL), saturated sodium bicarbonate (50 mL), and saturated brine (50 mL), dried over anhydrous sodium sulfate, filtered, and concentrated. The crude product was separated by column chromatography (PE/EA=3/1) to give a yellow oily liquid 18G (0.43 g, 82.6% yield). MS-ESI: m/z 339.0[M+H]⁺.

Step 6 18G (0.43 g, 1.27 mmol) was added into a 50-mL single-neck flask, before POCl₃ (5.1 g, 33.1 mmol) was slowly added in an ice bath. DIPEA (0.43 g, 3.31 mmol) was slowly and dropwise added with the internal temperature controlled at 15° C. The system was stirred at 100° C. for 2 h until LCMS indicated the completion of the reaction. The reaction solution was cooled to room temperature, concentrated by rotary evaporation at reduced pressure to remove the solvent, and poured slowly into ice water (50 mL). The mixture was adjusted to pH 9 with solid potassium carbonate and the aqueous phase was extracted with ethyl acetate (3×30 mL). The organic phase was collected, washed with saturated brine (50 mL), dried over anhydrous sodium sulfate, filtered, concentrated by rotary evaporation, and purified by column chromatography (PE/EA=10:1) to give a yellow oil 18H (0.32 g, 71.1% yield). MS-ESI: m/z 357.0[M+H]⁺.

Step 7 At room temperature, 18H (0.32 g, 0.9 mmol) and toluene (5 mL) were added into a 50-mL single-neck flask, before 2,4-dimethoxybenzylamine (1.5 g, 9 mmol) and 2-chlorobenzoic acid (0.14 g, 0.9 mmol) were sequentially added at room temperature. The reaction system was stirred at 120° C. overnight until LCMS indicated the completion of the reaction. The reaction solution was concentrated by rotary evaporation at reduced pressure to remove the solvent. 10 mL of water was added, and the mixture was extracted with EA (3×30 mL). The organic phase was collected, washed with saturated brine (30 mL), dried over anhydrous sodium sulfate, filtered, and concentrated. The crude product was purified by reversed-phase column chromatography (neutral separation system) to give a yellow solid 181 (0.14 g, 32.5% yield). MS-ESI: m/z 488.0[M+H]⁺.

Step 8 At room temperature, 18I (0.14 g, 0.28 mmol) and DMF (5 mL) were added into a 50-mL single-neck flask, before 18J (0.102 g, 0.56 mmol), Pd(PPh₃)₂Cl₂ (0.02 g, 0.028 mmol), and Cu₂O (0.12 g, 0.84 mmol) were sequentially added at room temperature. The system was stirred at 110° C. in an N₂ atmosphere overnight until LCMS indicated the completion of the reaction. 20 mL of water was added to the reaction solution and the mixture was extracted with EA (3×30 mL). The organic phase was collected, washed with saturated brine (20 mL), dried over anhydrous sodium sulfate, filtered, and concentrated. The crude product was separated by column chromatography (PE/EA=3/1) to give a yellow oily liquid 18K (0.12 g, 70.5% yield). MS-ESI: m/z 591.2[M+H]⁺.

Step 9 At room temperature, 18K (0.12 g, 0.2 mmol) and THE (10 mL) were added into a 50-mL single-neck flask, before Pd/C (50 mg) was added at room temperature. The system was purged 3 times with H₂ and stirred at 25° C. overnight until LCMS indicated the completion of the reaction. The reaction solution was filtered through celite and rinsed with THE (3×20 mL). The filtrate was concentrated by rotary evaporation at reduced pressure, and the crude product was separated by column chromatography (PE/EA=5/1) to give a yellow oily liquid 18L (0.1 g, 83.3% yield). MS-ESI: m/z 595.3 [M+H]⁺.

Step 10 At room temperature, 18L (0.1 g, 0.17 mmol) was added into a 50-mL single-neck flask, before TFA (3 mL) was slowly added in an ice bath. The system was stirred at 80° C. for 15 min until LCMS indicated the completion of the reaction. The reaction solution was concentrated by rotary evaporation at reduced pressure to remove the solvent and purified by reversed-phase column chromatography (neutral separation system) to give a white solid 18 (18 mg, 31.1% yield). MS-ESI: m/z 345.2[M+H]⁺.

¹H NMR (400 MHz, Methanol-d₄) δ 7.59 (dd, J=8.3, 1.3 Hz, 1H), 7.49 (dd, J=8.4, 7.2 Hz, 1H), 7.23 (dd, J=7.2, 1.3 Hz, 1H), 4.98 (s, 2H), 3.77 (q, J=7.0 Hz, 2H), 3.19-3.11 (m, 2H), 2.78 (t, J=6.9 Hz, 2H), 1.82-1.70 (m, 2H), 1.69-1.55 (m, 4H), 1.33 (t, J=7.0 Hz, 3H).

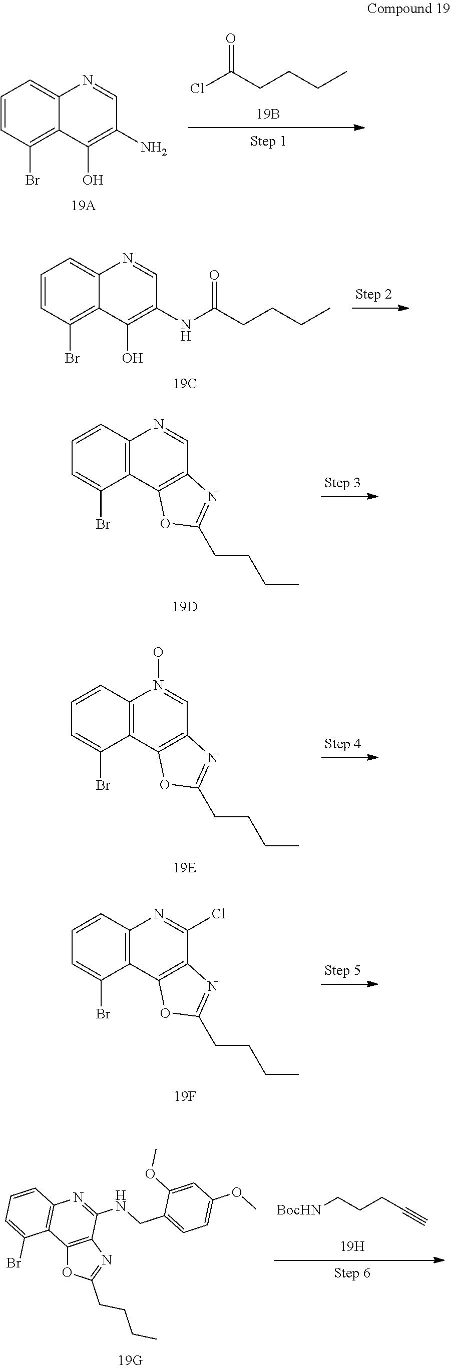

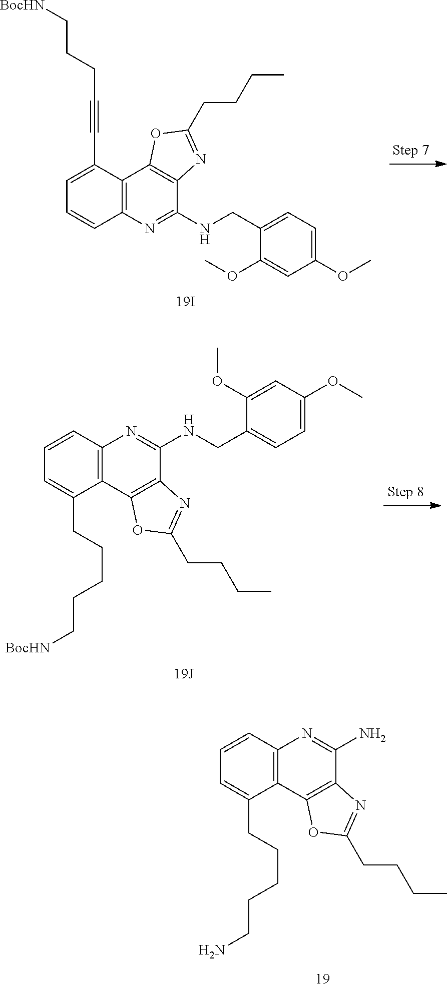 [SMILES: CCCCc1nc2c(NCc3ccc(OC)cc3OC)nc3cccc(C#CCCCNC(=O)OC(C)(C)C)c3c2o1 ; CCCCc1nc2c(NCc3ccc(OC)cc3OC)nc3cccc(CCCCCNC(=O)OC(C)(C)C)c3c2o1 ; CCCCc1nc2c(N)nc3cccc(CCCCCN)c3c2o1]

Step 1 19A (2 g, 8.4 mmol) and DCM (100 mL) were added into a 250-mL three-neck flask, before TEA (1.27 g, 12.6 mmol) was added. 19B (1.21 g, 10.08 mmol) was slowly added in an ice bath and the reaction system was stirred for 1 h at 25° C. until LCMS indicated the completion of the reaction. The reaction solution was poured into water (20 mL) and extracted with EA (3×50 mL). The organic phase was collected, washed with saturated brine (20 mL), dried over anhydrous sodium sulfate, filtered, and concentrated. The crude product was separated by column chromatography (PE/EA=3/1) to give a yellow solid 19C (1.4 g, 51.8% yield). MS-ESI: m/z 323.0[M+H]⁺.

Step 2 At room temperature, 19C (1.4 g, 4.3 mmol) and pyridine (60 mL) were added into a 100-mL single-neck flask, before P₂O₅ (6.1 g, 43 mmol) was added. The reaction system was stirred at 120° C. overnight until LCMS indicated the completion of the reaction. The reaction solution was concentrated by rotary evaporation to remove the solvent, poured into water (100 mL), and extracted with EA (3×50 mL). The organic phase was collected, washed with saturated brine (50 mL), dried over anhydrous sodium sulfate, filtered, and concentrated. The crude product was separated by chromatography (PE/EA=10/1) to give a yellow solid 19D (0.5 g, 37.8% yield). MS-ESI: m/z 305.1[M+H]⁺.

Step 3 At room temperature, 19D (0.5 g, 1.6 mmol) and CHCl₃ (10 mL) were added into a 50-mL single-neck flask, before m-CPBA (0.65 g, 3.2 mmol) was slowly added in an ice bath. The reaction system was stirred at 25° C. for 2 h until LCMS indicated the completion of the reaction. The reaction solution was poured into DCM (50 mL). The mixture was sequentially washed with 5% sodium thiosulfate (50 mL), saturated sodium bicarbonate (50 mL), and saturated brine (50 mL), dried over anhydrous sodium sulfate, filtered, and concentrated. The crude product was separated by column chromatography (PE/EA=3/1) to give a yellow oily liquid 19E (0.4 g, 76% yield). MS-ESI: m/z 321.0[M+H]⁺.

Step 4 19E (0.4 g, 1.25 mmol) was added into a 250-mL three-neck flask, before POCl₃ (5.75 g, 37.5 mmol) was slowly added in an ice bath. DIPEA (0.48 g, 3.75 mmol) was slowly and dropwise added with the internal temperature controlled at 15° C. The system was stirred at 100° C. for 2 h until LCMS indicated the completion of the reaction. The reaction solution was cooled to room temperature, concentrated by rotary evaporation at reduced pressure to remove the solvent, and poured slowly into ice water (50 mL). The mixture was adjusted to pH 9 with solid potassium carbonate and the aqueous phase was extracted with ethyl acetate (3×30 mL). The organic phase was collected, washed with saturated brine (50 mL), dried over anhydrous sodium sulfate, filtered, concentrated by rotary evaporation, and purified by column chromatography (PE/EA=10:1) to give a yellow oily liquid 19F (0.35 g, 83.3% yield). MS-ESI: m/z 339.0[M+H]⁺.

Step 5 At room temperature, 19F (0.35 g, 1.04 mmol) and toluene (5 mL) were added into a 50-mL single-neck flask, before 2,4-dimethoxybenzylamine (1.74 g, 10.4 mmol) and 2-chlorobenzoic acid (0.16 g, 1.04 mmol) were sequentially added at room temperature. The reaction system was stirred at 120° C. overnight until LCMS indicated the completion of the reaction. The reaction solution was concentrated by rotary evaporation at reduced pressure to remove the solvent. 10 mL of water was added, and the mixture was extracted with EA (3×30 mL). The organic phase was collected, washed with saturated brine (30 mL), dried over anhydrous sodium sulfate, filtered, and concentrated. The crude product was purified by reversed-phase column chromatography (neutral separation system) to give a yellow solid 19G (0.2 g, 41.1% yield). MS-ESI: m/z 470.0[M+H]⁺.

Step 6 At room temperature, 19G (0.2 g, 0.43 mmol) and DMF (5 mL) were added into a 50-mL single-neck flask, before 19H (0.16 g, 0.86 mmol), Pd(PPh₃)₂Cl₂ (0.03 g, 0.043 mmol), and Cu₂O (0.18 g, 1.29 mmol) were sequentially added at room temperature. The system was stirred at 110° C. in an N₂ atmosphere overnight until LCMS indicated the completion of the reaction. 20 mL of water was added to the reaction solution and the mixture was extracted with EA (3×30 mL). The organic phase was collected, washed with saturated brine (20 mL), dried over anhydrous sodium sulfate, filtered, and concentrated. The crude product was separated by column chromatography (PE/EA=3/1) to give a yellow oily liquid 191 (0.12 g, 49.4% yield). MS-ESI: m/z 573.4[M+H]⁺.

Step 7 At room temperature, 191 (0.12 g, 0.21 mmol) and THF (10 mL) were added into a 50-mL single-neck flask, before Pd/C (50 mg) was added at room temperature. The system was purged 3 times with H₂ and stirred at 25° C. overnight until LCMS indicated the completion of the reaction. The reaction solution was filtered through celite and rinsed with THF (3×20 mL). The filtrate was concentrated by rotary evaporation at reduced pressure, and the crude product was separated by column chromatography (PE/EA=5/1) to give a yellow oily liquid 19J (96 mg, 80% yield). MS-ESI: m/z 577.4[M+H]⁺.

Step 8 At room temperature, 19J (0.096 g, 0.17 mmol) was added into a 50-mL single-neck flask, before TFA (3 mL) was slowly added in an ice bath. The system was stirred at 80° C. for 15 min until LCMS indicated the completion of the reaction. The reaction solution was concentrated by rotary evaporation at reduced pressure to remove the solvent and purified by reversed-phase column chromatography (neutral separation system) to give a white solid 19 (22 mg, 40.5% yield). MS-ESI: m/z 327.3[M+H]⁺.

¹H NMR (400 MHz, Methanol-d₄) δ 7.54 (dd, J=8.5, 1.2 Hz, 1H), 7.45 (dd, J=8.5, 7.1 Hz, 1H), 7.16 (d, J=7.1 Hz, 1H), 3.25-3.17 (m, 2H), 3.08 (t, J=7.4 Hz, 2H), 2.69 (t, J=7.0 Hz, 2H), 1.99-1.88 (m, 2H), 1.79-1.68 (m, 2H), 1.64-1.44 (m, 6H), 1.02 (t, J=7.4 Hz, 3H).

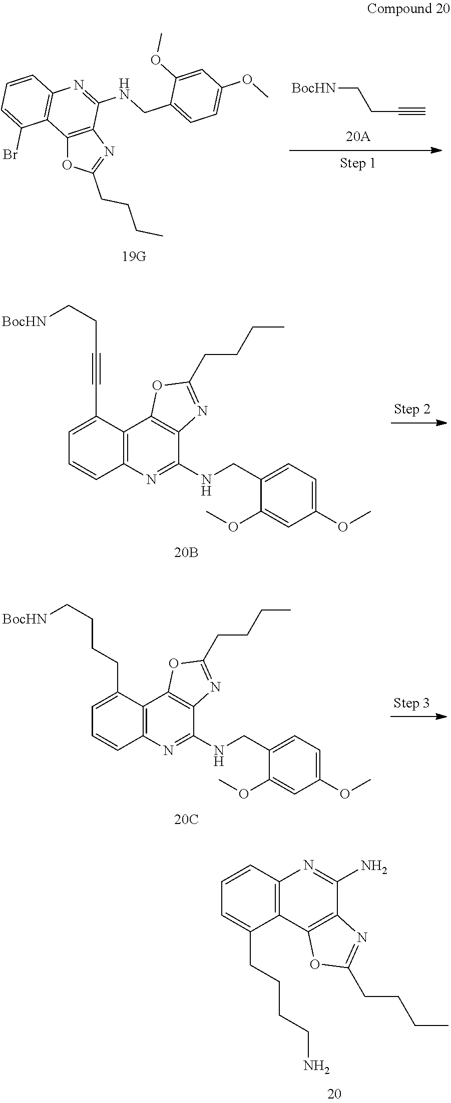 [SMILES: CCCCc1nc2c(NCc3ccc(OC)cc3OC)nc3cccc(Br)c3c2o1 ; C#CCCNC(=O)OC(C)(C)C ; CCCCc1nc2c(NCc3ccc(OC)cc3OC)nc3cccc(C#CCCNC(=O)OC(C)(C)C)c3c2o1 ; CCCCc1nc2c(NCc3ccc(OC)cc3OC)nc3cccc(CCCCNC(=O)OC(C)(C)C)c3c2o1 ; CCCCc1nc2c(N)nc3cccc(CCCCN)c3c2o1]

Step 1 At room temperature, 19G (0.28 g, 0.6 mmol) and DMF (6 mL) were added into a 50-mL single-neck flask, before 20A (0.254 g, 1.5 mmol), Pd(PPh₃)₂Cl₂ (0.042 g, 0.06 mmol), and Cu₂O (0.257 g, 1.8 mmol) were sequentially added at room temperature. The system was stirred at 110° C. in an N₂ atmosphere overnight until LCMS indicated the completion of the reaction. 20 mL of water was added to the reaction solution and the mixture was extracted with EA (3×30 mL). The organic phase was collected, washed with saturated brine (20 mL), dried over anhydrous sodium sulfate, filtered, and concentrated. The crude product was separated by column chromatography (PE/EA=3/1) to give a yellow oily liquid 20B (140 mg, 42% yield). MS-ESI: m/z 559.2[M+H]⁺.

Step 2 At room temperature, 20B (0.14 g, 0.25 mmol) and THF (10 mL) were added into a 50-mL single-neck flask, before Pd/C (50 mg) was added at room temperature. The system was purged 3 times with H₂ and stirred at 25° C. overnight until LCMS indicated the depletion of the starting materials. The reaction solution was filtered through celite and rinsed with THF (3×20 mL). The filtrate was concentrated by rotary evaporation at reduced pressure, and the crude product was separated by column chromatography (PE/EA=5/1) to give a yellow oily liquid 20C (120 mg, 85% yield). MS-ESI: m/z 563.4[M+H]⁺.

Step 3 At room temperature, 20C (0.12 g, 0.21 mmol) was added into a 50-mL single-neck flask, before TFA (4 mL) was slowly added in an ice bath. The system was stirred at 80° C. for 15 min until LCMS indicated the completion of the reaction. The reaction solution was concentrated by rotary evaporation at reduced pressure and subjected to preparative reversed-phase chromatography (TFA separation system) to give a white solid 20 (4.2 mg, 6.3% yield). MS-ESI: m/z 313.2[M+H]⁺.

¹H NMR (400 MHz, Methanol-d₄) δ 7.76-7.66 (m, 2H), 7.47 (dd, J=6.9, 1.5 Hz, 1H), 3.36-3.32 (m, 2H), 3.15 (t, J=7.5 Hz, 2H), 3.04-2.95 (m, 2H), 2.02-1.92 (m, 2H), 1.90-1.77 (m, 4H), 1.61-1.49 (m, 2H), 1.03 (t, J=7.4 Hz, 3H).

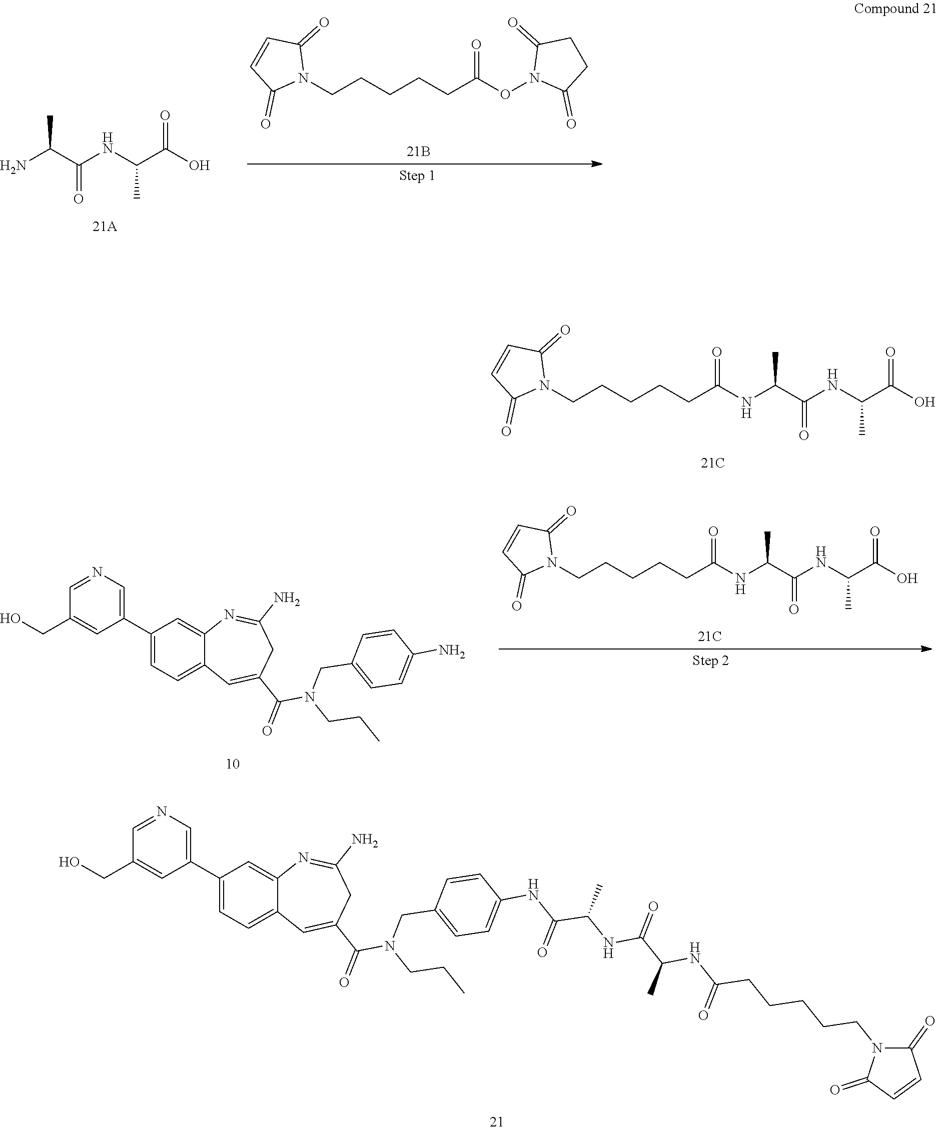 [SMILES: C[C@H](N)C(=O)N[C@@H](C)C(=O)O ; O=C(CCCCCN1C(=O)C=CC1=O)ON1C(=O)CCC1=O ; C[C@H](NC(=O)[C@H](C)NC(=O)CCCCCN1C(=O)C=CC1=O)C(=O)O ; CCCN(Cc1ccc(N)cc1)C(=O)C1=Cc2ccc(-c3cncc(CO)c3)cc2N=C(N)C1 ; C[C@H](NC(=O)[C@H](C)NC(=O)CCCCCN1C(=O)C=CC1=O)C(=O)O ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1]

Step 1 21A (3.3 g, 20.60 mmol, 1 eq) and 21B (7 g, 22.66 mmol, 1.1 eq) were dissolved in a mixed solvent of acetonitrile (30 mL) and water (60 mL). The system was stirred at room temperature overnight in an argon atmosphere. LCMS indicated that some of the starting materials remained unreacted. The reaction solution was directly injected into a reversed-phase column (mobile phase: water and acetonitrile), and under the condition of 50% acetonitrile, a product was obtained, which was then lyophilized to give a white solid 21C (2 g, 27.5% yield). MS-ESI: m/z 354.2 [M+H]⁺.

Step 2 10 (180 mg, 0.366 mmol, 1 eq) and 21C (130 mg, 0.366 mmol, 1 eq) were added to a mixed solvent of DCM (5 mL) and DMA (5 mL). The system was purged twice with argon before EDCI (281 mg, 1.46 mmol, 4 eq) was added in one portion at room temperature, then purged twice again with argon, and stirred at room temperature overnight until LCMS indicated the depletion of the starting materials. The mixture was directly subjected to preparative chromatography and lyophilized to give a white solid 21 (30 mg, 11.5% yield). MS-ESI: m/z 791.4 [M+H]⁺.

¹H NMR (400 MHz, Chloroform-d) δ 8.72 (d, J=2.3 Hz, 1H), 8.48 (d, J=1.9 Hz, 1H), 8.00-7.95 (m, 1H), 7.58 (d, J=8.4 Hz, 2H), 7.38 (s, 1H), 7.33-7.15 (m, 4H), 6.91 (s, 1H), 6.84 (s, 1H), 4.63-4.51 (m, 4H), 4.32 (q, J=7.1 Hz, 1H), 4.19 (q, J=7.1 Hz, 1H), 3.34 (t, J=7.0 Hz, 2H), 3.30-3.10 (m, 3H), 2.77 (s, 2H), 2.15-1.98 (m, 3H), 1.59-1.38 (m, 6H), 1.29 (d, J=7.1 Hz, 3H), 1.24-1.06 (m, 6H), 0.75 (brs, 3H).

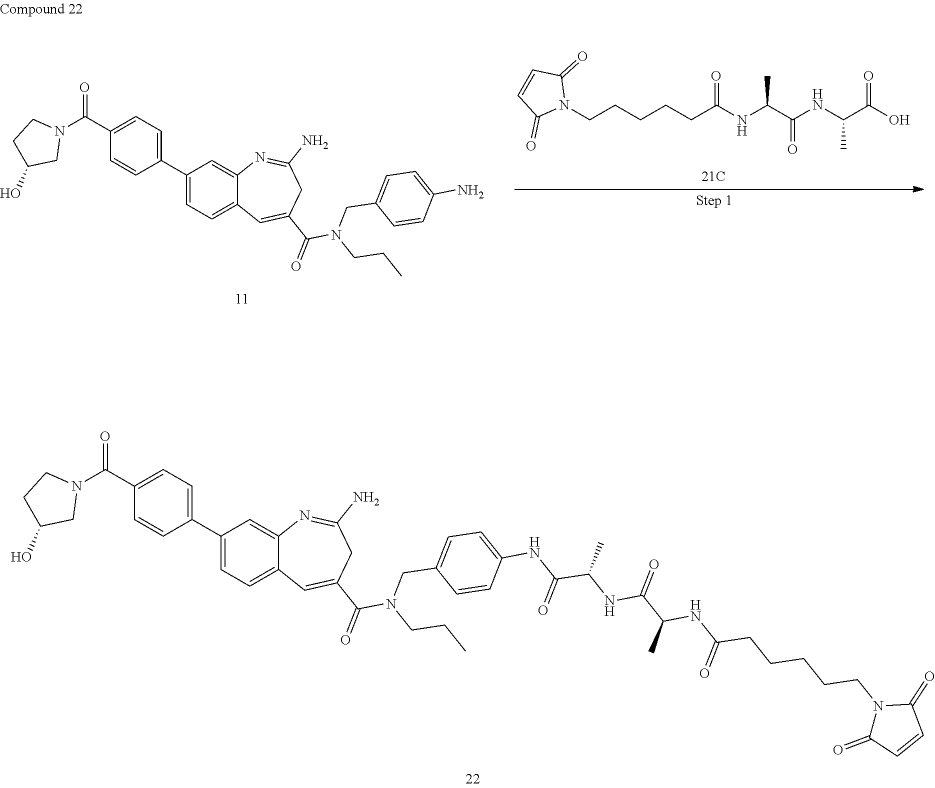 [SMILES: CCCN(Cc1ccc(N)cc1)C(=O)C1=Cc2ccc(-c3ccc(C(=O)N4CC[C@@H](O)C4)cc3)cc2N=C(N)C1 ; C[C@H](NC(=O)[C@H](C)NC(=O)CCCCCN1C(=O)C=CC1=O)C(=O)O ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc(C(=O)N4CC[C@@H](O)C4)cc3)cc2N=C(N)C1]

Step 1 11 (130 mg, 0.226 mmol, 1 eq) and 21C (80 mg, 0.226 mmol, 1 eq) were added to a mixed solvent of DCM (5 mL) and DMA (5 mL). The system was purged twice with argon before EDCI (174 mg, 0.906 mmol, 4 eq) was added in one portion at room temperature, then purged twice again with argon, and stirred at room temperature overnight until LCMS indicated the depletion of the starting materials. The mixture was directly subjected to preparative chromatography and lyophilized to give a white solid 22 (13 mg, 6.6% yield). MS-ESI: m/z 873.4 [M+H]⁺.

¹H NMR (400 MHz, DMSO-d₆) δ 9.87 (s, 1H), 8.06 (d, J=7.1 Hz, 1H), 7.99 (d, J=7.0 Hz, 1H), 7.73 (d, J=8.1 Hz, 2H), 7.66-7.55 (m, 4H), 7.42-7.34 (m, 1H), 7.34-7.29 (m, 1H), 7.29-7.17 (m, 3H), 6.99 (s, 2H), 6.92-6.80 (m, 3H), 5.85-5.72 (m, 1H), 4.97 (dd, J=33.8, 3.4 Hz, 1H), 4.58 (s, 2H), 4.44-4.19 (m, 3H), 3.67-3.51 (m, 3H), 3.42-3.37 (m, 2H), 3.24-3.17 (m, 2H), 3.03-2.91 (m, 2H), 2.84-2.75 (m, 2H), 2.27-2.02 (m, 5H), 2.01-1.74 (m, 2H), 1.59-1.41 (m, 7H), 1.30 (d, J=7.1 Hz, 3H), 1.24-1.15 (m, 5H), 0.77 (brs, 3H).

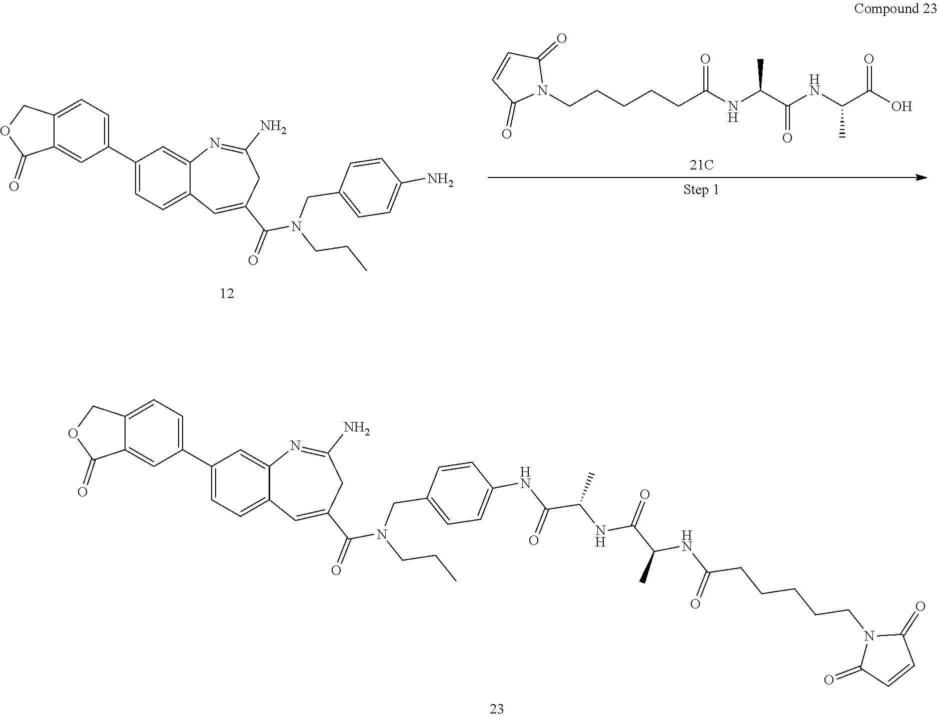 [SMILES: CCCN(Cc1ccc(N)cc1)C(=O)C1=Cc2ccc(-c3ccc4c(c3)C(=O)OC4)cc2N=C(N)C1 ; C[C@H](NC(=O)[C@H](C)NC(=O)CCCCCN1C(=O)C=CC1=O)C(=O)O ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc4c(c3)C(=O)OC4)cc2N=C(N)C1]

Step 1 12 (130 mg, 0.251 mmol, 1 eq) and 21C (89 mg, 0.251 mmol, 1 eq) were added to a mixed solvent of DCM (5 mL) and DMA (5 mL). The system was purged twice with argon before EDCI (193 mg, 1.01 mmol, 4 eq) was added in one portion at room temperature, then purged twice again with argon, and stirred at room temperature overnight until LCMS indicated the depletion of the starting materials. The mixture was directly subjected to preparative chromatography and lyophilized to give a white solid 23 (15 mg, 7.3% yield).

MS-ESI: m/z 816.4 [M+H]⁺.

¹H NMR (400 MHz, DMSO-d₆) δ 9.87 (s, 1H), 8.14-7.94 (m, 4H), 7.75 (d, J=8.0 Hz, 1H), 7.61 (d, J=8.6 Hz, 2H), 7.45-7.35 (m, 1H), 7.35-7.29 (m, 2H), 7.29-7.19 (m, 2H), 6.99 (s, 2H), 6.93-6.82 (m, 3H), 5.46 (s, 2H), 4.58 (s, 2H), 4.37 (p, J=6.9 Hz, 1H), 4.25 (p, J=7.1 Hz, 1H), 3.39-3.36 (m, 2H), 3.21 (d, J=7.8 Hz, 2H), 2.80 (s, 2H), 2.10 (t, J=7.4 Hz, 2H), 1.58-1.42 (m, 6H), 1.30 (d, J=7.1 Hz, 3H), 1.25-1.13 (m, 5H), 0.77 (s, 3H).

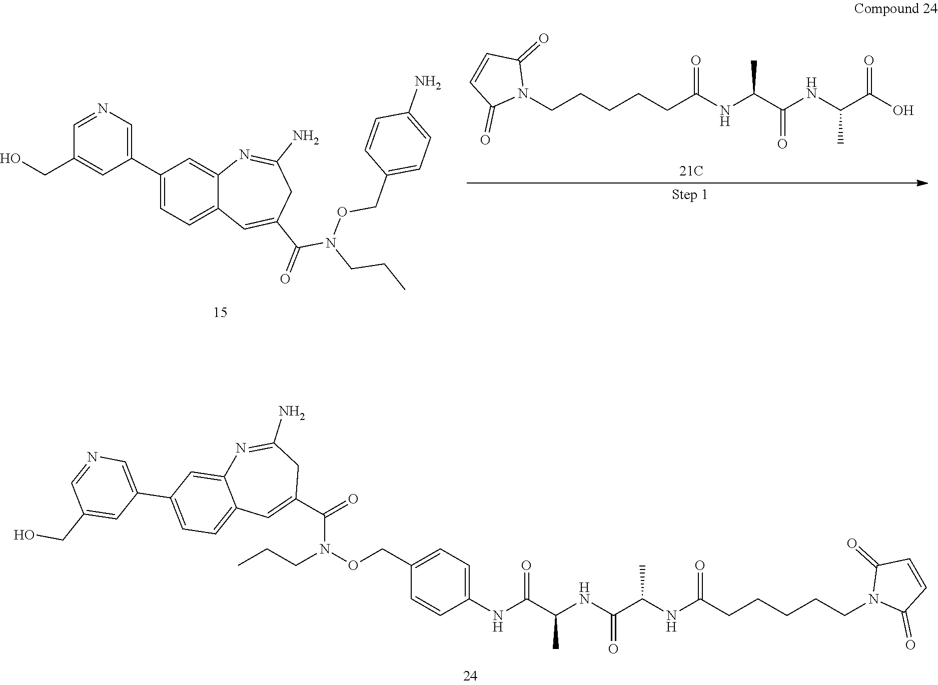 [SMILES: CCCN(OCc1ccc(N)cc1)C(=O)C1=Cc2ccc(-c3cncc(CO)c3)cc2N=C(N)C1 ; C[C@H](NC(=O)[C@H](C)NC(=O)CCCCCN1C(=O)C=CC1=O)C(=O)O ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1]

Step 1 15 (100 mg, 0.212 mmol, 1 eq) was prepared into its hydrochloride and then added, together with 21C (75 mg, 0.212 mmol, 1 eq), to a mixed solvent of DCM (5 mL) and DMA (5 mL). The system was purged twice with argon before EDCI (163 mg, 0.848 mmol, 4 eq) was added in one portion at room temperature, then purged twice again with argon, and stirred at room temperature overnight until LCMS indicated the completion of the reaction. The mixture was directly subjected to preparative chromatography and lyophilized to give a white solid 24 (14 mg, 8.2% yield).

MS-ESI: m/z 807.4 [M+H]⁺.

¹H NMR (400 MHz, DMSO-d₆) δ 9.90 (s, 1H), 8.79 (d, J=2.3 Hz, 1H), 8.51 (d, J=1.9 Hz, 1H), 8.08 (d, J=7.2 Hz, 1H), 8.04-7.97 (m, 2H), 7.53 (d, J=8.4 Hz, 2H), 7.39 (d, J=8.2 Hz, 1H), 7.35 (d, J=1.9 Hz, 1H), 7.28 (dd, J=8.0, 2.0 Hz, 1H), 7.21 (d, J=8.3 Hz, 2H), 7.07 (s, 1H), 6.99 (s, 2H), 6.87 (s, 2H), 5.40 (t, J=5.8 Hz, 1H), 4.75 (s, 2H), 4.61 (d, J=5.5 Hz, 2H), 4.39-4.29 (m, 1H), 4.27-4.18 (m, 1H), 3.64 (t, J=7.0 Hz, 2H), 2.78 (s, 2H), 2.08 (t, J=7.5 Hz, 2H), 1.71-1.59 (m, 2H), 1.51-1.39 (m, 4H), 1.27 (d, J=7.1 Hz, 3H), 1.21-1.11 (m, 5H), 0.90 (t, J=7.4 Hz, 3H).

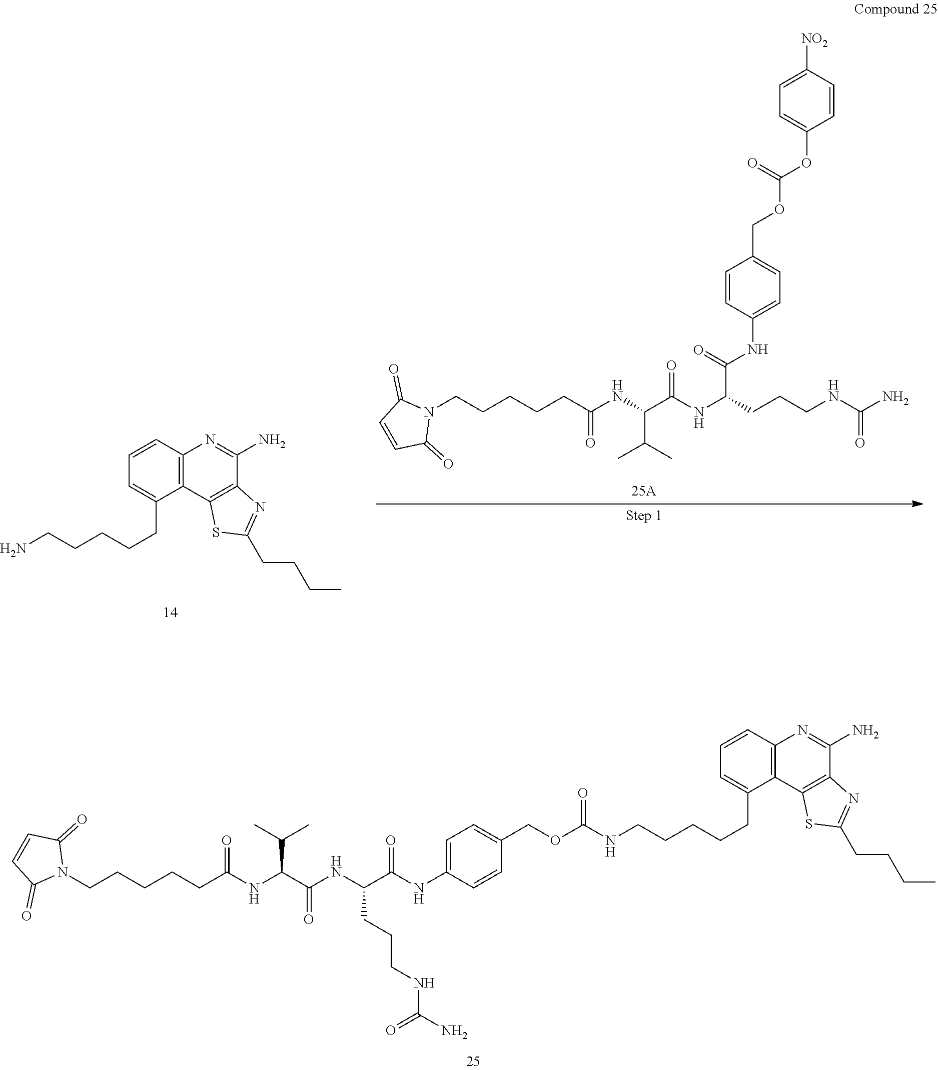 [SMILES: CCCCc1nc2c(N)nc3cccc(CCCCCN)c3c2s1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCCCc1nc2c(N)nc3cccc(CCCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

Step 1 At room temperature, 14 (65 mg, 0.19 mmol) was added into a 100-mL single-neck flask and dissolved in DMF (10 mL), before DIEA (74 mg, 0.57 mmol) and 15A (140 mg, 0.19 mmol) were sequentially added. The reaction system was stirred at 25° C. overnight until LCMS indicated the completion of the reaction. The reaction solution was directly purified by reversed-phase column chromatography (neutral separation system) to give a white solid 25 (11 mg, 6.1% yield).

MS-ESI. m/z 941.4 [M+H]⁺.

¹H NMR (400 MHz, Methanol-d₄) δ 7.61-7.53 (m, 3H), 7.49-7.43 (m, 1H), 7.30 (d, J=8.2 Hz, 2H), 7.21 (d, J=7.1 Hz, 1H), 6.77 (s, 2H), 5.02 (s, 2H), 4.49 (dd, J=9.0, 5.1 Hz, 1H), 4.15 (d, J=7.4 Hz, 1H), 3.46 (t, J=7.1 Hz, 2H), 3.24-3.04 (m, 8H), 2.26 (t, J=7.4 Hz, 2H), 2.11-2.00 (m, 1H), 1.97-1.82 (m, 3H), 1.80-1.67 (m, 3H), 1.67-1.44 (m, 12H), 1.34-1.24 (m, 2H), 1.01 (t, J=7.4 Hz, 3H), 0.95 (dd, J=6.8, 3.2 Hz, 6H).

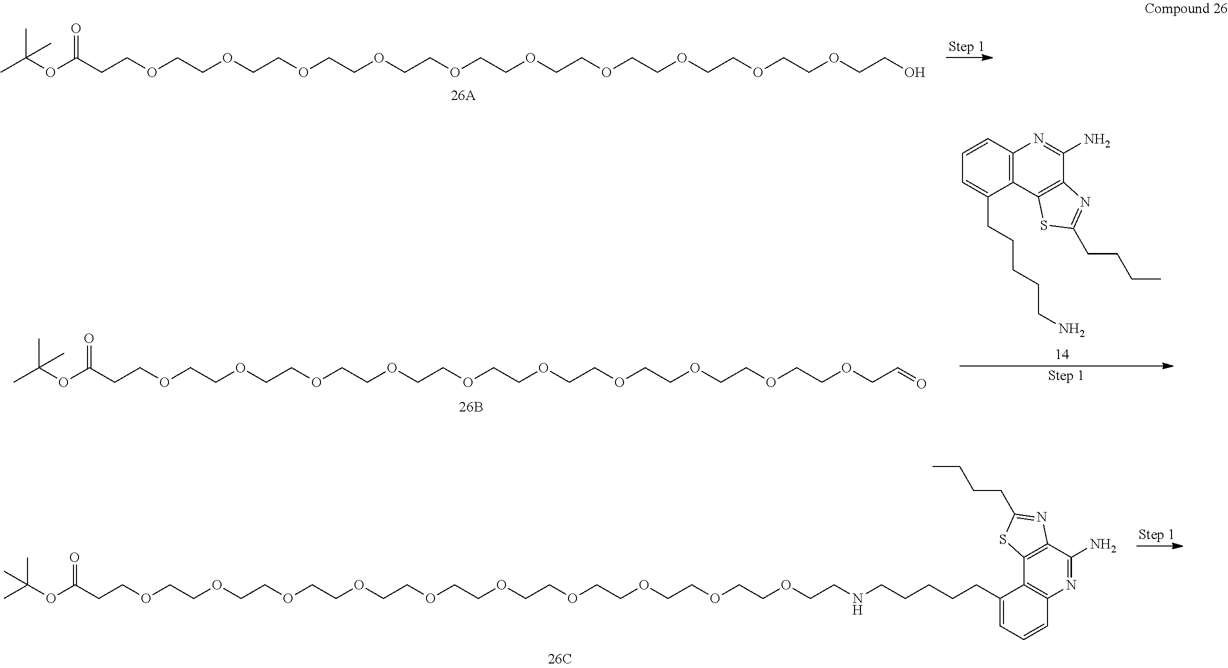 [SMILES: CC(C)(C)OC(=O)CCOCCOCCOCCOCCOCCOCCOCCOCCOCCOCCO ; CCCCc1nc2c(N)nc3cccc(CCCCCN)c3c2s1 ; CC(C)(C)OC(=O)CCOCCOCCOCCOCCOCCOCCOCCOCCOCCOCC=O ; CCCCc1nc2c(N)nc3cccc(CCCCCNCCOCCOCCOCCOCCOCCOCCOCCOCCOCCOCCC(=O)OC(C)(C)C)c3c2s1]

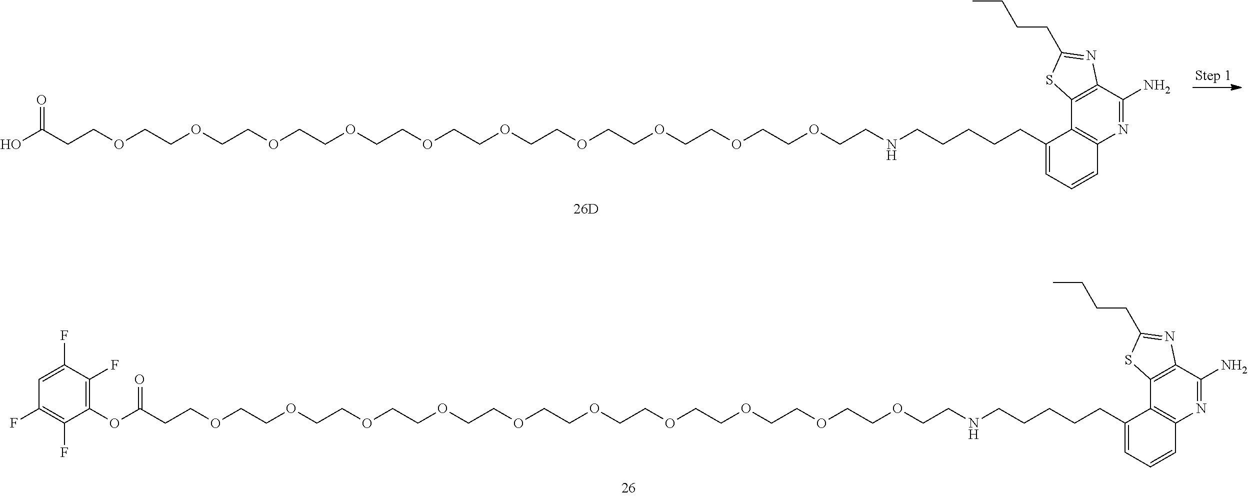 [SMILES: CCCCc1nc2c(N)nc3cccc(CCCCCNCCOCCOCCOCCOCCOCCOCCOCCOCCOCCOCCC(=O)O)c3c2s1 ; CCCCc1nc2c(N)nc3cccc(CCCCCNCCOCCOCCOCCOCCOCCOCCOCCOCCOCCOCCC(=O)Oc4c(F)c(F)cc(F)c4F)c3c2s1]

Step 1 At room temperature, oxalyl chloride (129.5 mg, 1.02 mmol) and DCM (5 mL) were added into a 100-mL three-neck flask. The reaction solution was cooled to −78° C. in an N₂ atmosphere, before DMSO (159.4 mg, 2.04 mmol, in 5 mL DCM) was slowly added. The reaction solution was then stirred at −78° C. for 15 min, before 26A (300 mg, 0.51 mmol, in 5 mL DCM) was slowly added. After stirring for another 15 min, Et₃N (309.6 mg, 3.06 mmol, in 5 mL DCM) was slowly added. The reaction system was stirred at −78° C. for another 15 min, then slowly warmed to 25° C., and incubated for more than 30 min until TLC indicated the depletion of the starting materials and the formation of a new spot. The reaction was quenched with saturated NH₄C₁, and the aqueous phase was extracted 3 times with DCM. The organic phase was collected, washed with saturated brine, dried over anhydrous sodium sulfate, filtered, and concentrated to give a yellow oily liquid 26B (250 mg, crude product, yield not calculated).

¹H NMR (400 MHz, Chloroform-d) δ 9.73 (s, 1H), 4.16 (d, J=0.8 Hz, 2H), 3.78-3.57 (m, 38H), 2.50 (t, J=6.4 Hz, 2H), 1.47-1.41 (m, 9H).

Step 2 At room temperature, 26B (250 mg, 0.43 mmol) was added to a 50-mL single-neck flask and dissolved in DMF (10 mL), before 14 (100 mg, 0.29 mmol) and AcOH (0.1 mL) were sequentially added. The reaction system was stirred at 25° C. for 2 h, before Na(OAc)₃BH (120 mg, 0.87 mmol) was added. The reaction system was then stirred overnight. When TLC indicated the completion of the reaction, the reaction solution was concentrated by rotary evaporation at reduced pressure to remove the solvent and then purified by column chromatography (EA/MeOH=10:1 to 5:1) to give a yellow oily liquid 26C (200 mg, 75.7% yield). MS-ESI: m/z 911.5 [M+H]⁺.

Step 3 At room temperature, 26C (200 mg, 0.22 mmol) was added into a 50-mL single-neck flask and dissolved in DCM (10 mL), before TFA (2 mL) was slowly added in an ice bath. The reaction system was stirred at 25° C. for 2 h until LCMS indicated the completion of the reaction. The reaction solution was concentrated by rotary evaporation at reduced pressure to remove the solvent and purified by reversed-phase column chromatography (TFA/ACN system) to give a color less oily liquid 26D (100 mg, 54.3% yield). MS-ESI: m/z 855.5 [M+H]⁺.

Step 4 At 25° C., 2,3,5,6-tetrafluorophenol (50 mg, 0.3 mmol) was added into a 50-mL single-neck flask and dissolved in DMF (10 mL), before EDCI (57.5 mg, 0.3 mmol) was added. The reaction solution was stirred for 5 min, before 26D (100 mg, 0.12 mmol) was added. The reaction system was stirred at 25° C. for 1 h until LCMS indicated the completion of the reaction. The reaction solution was directly purified by reversed-phase column chromatography (TFA/MeCN separation system) to give an off-white oily liquid 26 (25 mg, 20.8% yield).

MS-ESI: m/z 1003.5 [M+H]⁺.

¹H NMR (400 MHz, Methanol-d₄) δ 7.76-7.67 (m, 2H), 7.54-7.48 (m, 1H), 7.47-7.37 (m, 1H), 3.85 (t, J=5.9 Hz, 2H), 3.82-3.75 (m, 2H), 3.70-3.56 (m, 36H), 3.29-3.19 (m, 4H), 3.16-3.07 (m, 2H), 2.98-2.92 (m, 2H), 1.96 (p, J=7.7 Hz, 2H), 1.89-1.74 (m, 4H), 1.71-1.59 (m, 2H), 1.59-1.48 (m, 2H), 1.03 (t, J=7.4 Hz, 3H).

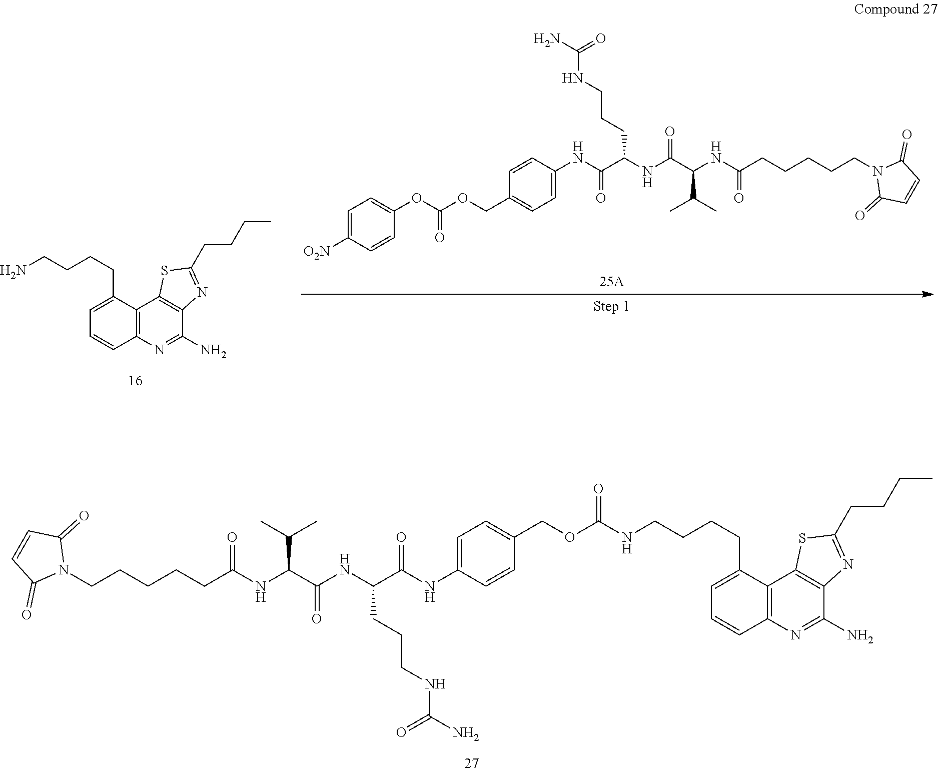 [SMILES: CCCCc1nc2c(N)nc3cccc(CCCCN)c3c2s1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

Step 1 At room temperature, 16 (42 mg, 0.13 mmol) was added into a 25-mL single-neck flask and dissolved in DMF (10 mL), before DIEA (50 mg, 0.39 mmol) and 25A (96 mg, 0.13 mmol) were sequentially added. The reaction system was stirred at 25° C. for 1 h until LCMS indicated the completion of the reaction. The reaction solution was directly purified by reversed-phase column chromatography (neutral reversed-phase separation system) to give a white solid 27 (7.7 mg, 6.4% yield).

MS-ESI: m/z 927.5[M+H]⁺.

¹H NMR (400 MHz, Methanol-d₄) δ 7.58 (d, J=8.3 Hz, 1H), 7.54 (d, J=8.2 Hz, 2H), 7.47 (t, J=7.8 Hz, 1H), 7.26 (d, J=8.2 Hz, 2H), 7.21 (d, J=7.2 Hz, 1H), 6.77 (s, 2H), 5.00 (s, 2H), 4.51 (dd, J=9.1, 5.1 Hz, 1H), 4.16 (d, J=7.4 Hz, 1H), 3.46 (t, J=7.2 Hz, 2H), 3.25-3.16 (m, 5H), 3.16-3.05 (m, 3H), 2.27 (t, J=7.4 Hz, 2H), 2.13-2.02 (m, 1H), 1.96-1.84 (m, 3H), 1.81-1.68 (m, 5H), 1.68-1.43 (m, 9H), 1.36-1.21 (m, 3H), 1.05-0.93 (m, 9H).

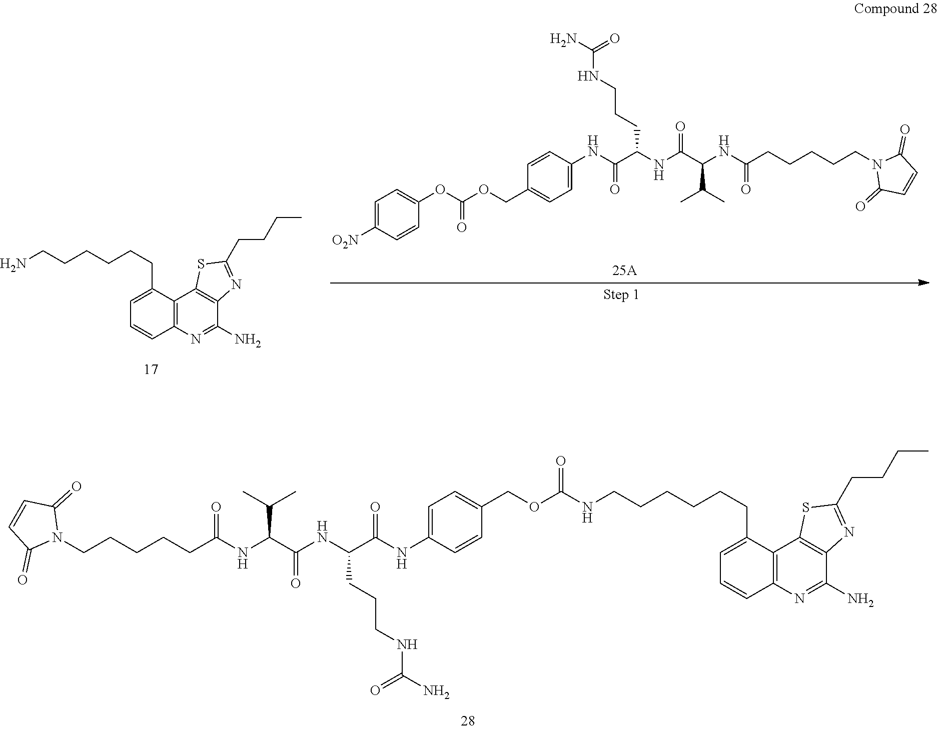 [SMILES: CCCCc1nc2c(N)nc3cccc(CCCCCCN)c3c2s1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCCCc1nc2c(N)nc3cccc(CCCCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

Step 1 At room temperature, 17 (50 mg, 0.14 mmol) was added into a 25-mL single-neck flask and dissolved in DMF (10 mL), before DIEA (54 mg, 0.42 mmol) and 25A (103 mg, 0.13 mmol) were sequentially added. The reaction system was stirred at 25° C. for 1 h until LCMS indicated the completion of the reaction. The reaction solution was directly purified by reversed-phase column chromatography (neutral reversed-phase separation system) to give a white solid 28 (9.2 mg, 6.9% yield).

MS-ESI: m/z 955.5[M+H]⁺.

¹H NMR (400 MHz, Methanol-d₄) δ 7.62-7.51 (m, 3H), 7.47 (t, J=7.7 Hz, 1H), 7.29 (d, J=8.2 Hz, 2H), 7.21 (d, J=7.3 Hz, 1H), 6.78 (s, 2H), 5.01 (s, 2H), 4.58 (s, 1H), 4.53-4.44 (m, 1H), 4.15 (d, J=7.5 Hz, 1H), 3.50-3.44 (m, 2H), 3.25-3.17 (m, 3H), 3.17-3.05 (m, 6H), 2.26 (t, J=7.5 Hz, 2H), 2.13-2.00 (m, 1H), 2.00-1.81 (m, 3H), 1.81-1.67 (m, 3H), 1.67-1.36 (m, 15H), 1.36-1.23 (m, 3H), 1.05-0.89 (m, 9H).

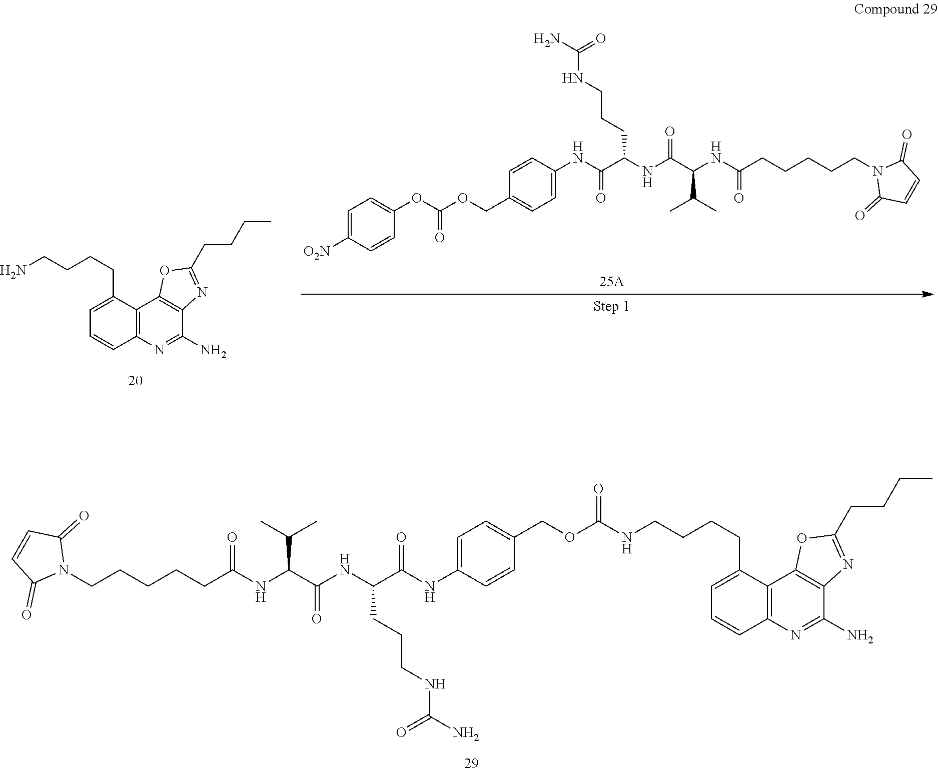 [SMILES: CCCCc1nc2c(N)nc3cccc(CCCCN)c3c2o1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2o1]

Step 1 At room temperature, 20 (34 mg, 0.063 mmol) was added into a 50-mL single-neck flask and dissolved in DMF (6 mL), before DIEA (20 mg, 0.157 mmol) and 25A (46 mg, 0.063 mmol) were sequentially added. The reaction system was stirred at 25° C. overnight until LCMS indicated the completion of the reaction. The reaction solution was directly subjected to preparative reversed-phase chromatography (NH₄HCO₃ system) to give a white solid 29 (27.1 mg, 27% yield).

MS-ESI. m/z 911.4 [M+H]⁺.

¹H NMR (400 MHz, Methanol-d₄) δ 7.59-7.51 (m, 3H), 7.49-7.43 (m, 1H), 7.27 (d, J=8.2 Hz, 2H), 7.16 (d, J=7.0 Hz, 1H), 6.77 (s, 2H), 5.00 (s, 2H), 4.51 (dd, J=8.9, 4.9 Hz, 1H), 4.16 (d, J=7.5 Hz, 1H), 3.49-3.44 (m, 2H), 3.26-3.15 (m, 5H), 3.15-3.03 (m, 3H), 2.27 (t, J=7.4 Hz, 2H), 2.13-2.02 (m, 1H), 1.97-1.84 (m, 3H), 1.80-1.54 (m, 11H), 1.54-1.44 (m, 2H), 1.35-1.27 (m, 2H), 1.03-0.94 (m, 9H).

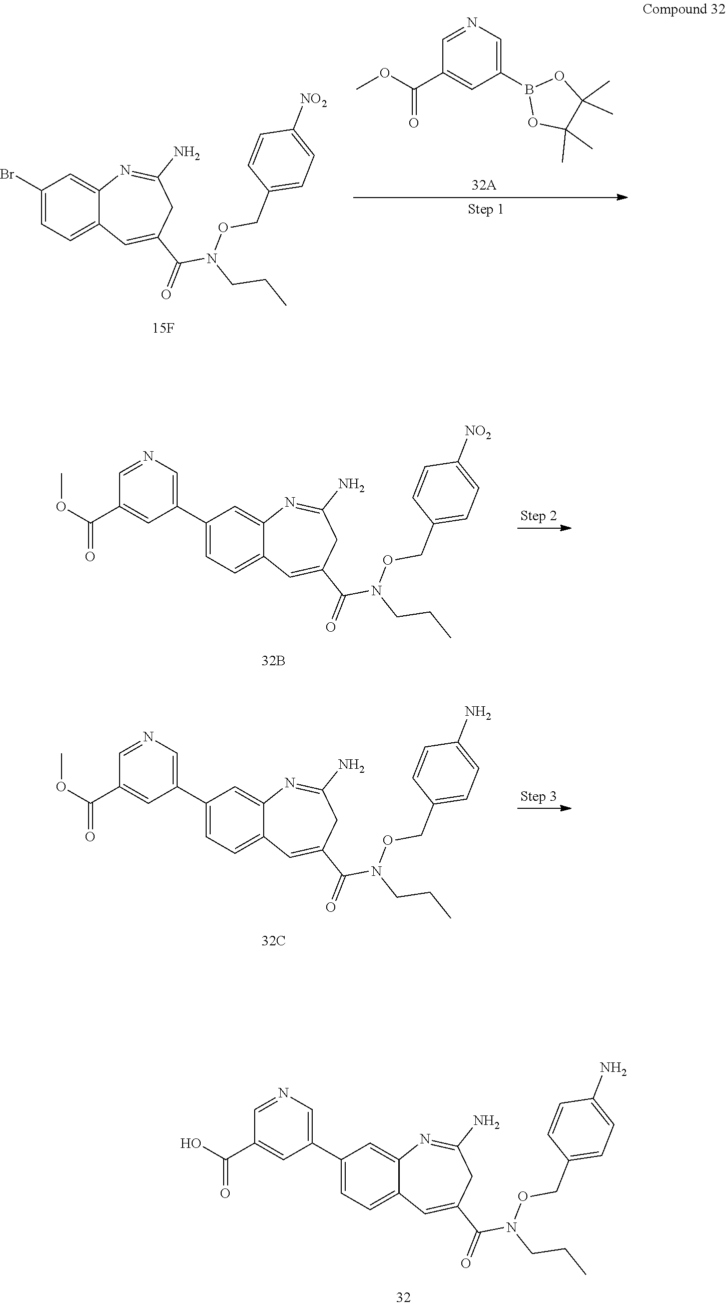

Step 1 Compound 15F (1.0 g, 2.11 mmol, 1 eq) was added to a mixed solvent of 1,4-dioxane (20 mL) and water (2 mL), before compound 32A (556 mg, 2.11 mmol, 1 eq) and anhydrous potassium phosphate (1.35 g, 6.34 mmol, 3 eq) were added. The system was purged twice with argon before Pd(PPh₃)₄ (100 mg) was added, then purged twice again with argon, heated to 90° C., and incubated for reaction for 2 h. When LCMS indicated the depletion of the starting materials, the reaction solution was poured into 100 mL of water and extracted three times with ethyl acetate (100 mL×3). The organic phases were combined, dried over anhydrous sodium sulfate, concentrated at reduced pressure, and purified by column chromatography (DCM:MeOH=20:1) to give a yellow solid 32B (400 mg, 36% yield). MS-ESI: m/z 530.2 [M+H]⁺.

Step 2 Compound 32B (400 mg, 0.756 mmol, 1 eq) was dissolved in methanol (10 mL), before Raney nickel (0.1 g) was added. The system was purged twice with hydrogen and stirred at room temperature for 3 h. When LCMS indicated remarkable product peaks, the mixture was filtered through celite, and the filtrate was directly used in the next reaction. MS-ESI: m/z 500.2 [M+H]⁺.

Step 3 THF (10 mL) and water (10 mL) were added to the reaction solution from the previous step, before lithium hydroxide monohydrate (91 mg, 2.27 mmol, 3 eq) was added. The system was stirred at room temperature for 3 h. When LCMS indicated the depletion of the starting materials, the mixture was concentrated at reduced pressure and adjusted to pH 5-6 with 1 N hydrochloric acid. The resultant mixture was filtered, and the yellow filter cake was subjected to preparative HPLC to give a white solid 32 (21 mg, 5.7% yield over the two steps). MS-ESI: m/z 486.2 [M+H]⁺.

¹H NMR (400 MHz, DMSO) δ 12.87-11.32 (m, 1H), 9.87 (s, 1H), 9.26 (s, 1H), 9.20 (d, J=2.2 Hz, 1H), 9.13-9.12 (d, J=1.8 Hz, 1H), 8.55-8.54 (t, J=2.0 Hz, 1H), 7.88-7.86 (d, J=8.2 Hz, 1H), 7.79 (s, 1H), 7.70-7.68 (d, J=8.3 Hz, 1H), 7.22 (s, 1H), 7.01-6.99 (d, J=8.2 Hz, 2H), 6.59-6.57 (d, J=8.1 Hz, 2H), 4.70 (s, 2H), 3.70 (s, 2H), 3.26 (s, 2H), 1.73-1.60 (m, 2H), 0.94-0.91 (t, J=7.4 Hz, 3H).

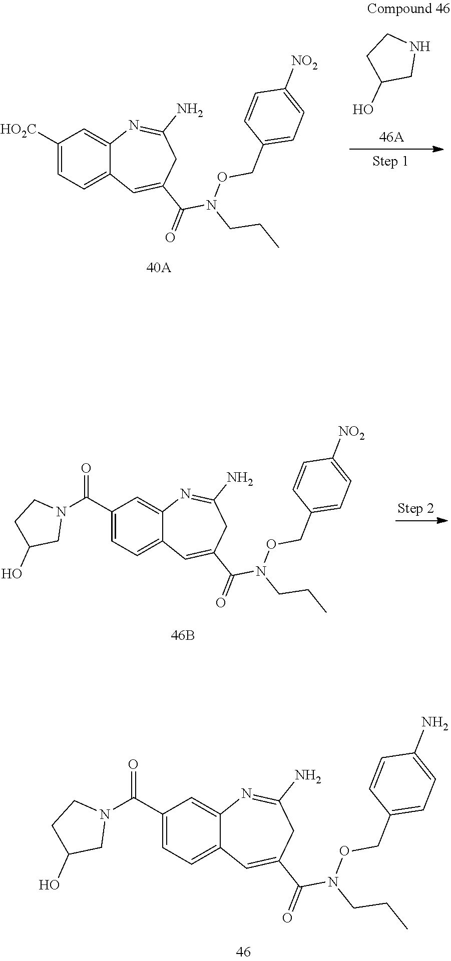 [SMILES: CCCN(OCc1ccc([N+](=O)[O-])cc1)C(=O)C1=Cc2ccc(C(=O)O)cc2N=C(N)C1 ; OC1CCNC1 ; CCCN(OCc1ccc([N+](=O)[O-])cc1)C(=O)C1=Cc2ccc(C(=O)N3CCC(O)C3)cc2N=C(N)C1 ; CCCN(OCc1ccc(N)cc1)C(=O)C1=Cc2ccc(C(=O)N3CCC(O)C3)cc2N=C(N)C1]

Step 1 Compound 40A (100 mg, 0.228 mmol, 1 eq.) and compound 46A (19.87 mg, 0.228 mmol, 1 eq.) were dissolved in DMA (3 mL), before EDCI (174.90 g, 0.912 mmol, 4 eq.) was added in one portion. The system was stirred at room temperature overnight. When LCMS indicated the depletion of compound 40A, the reaction solution was directly used in the next reaction without treatment. MS-ESI: m/z 508.2 [M+H]⁺.

Step 2 Raney nickel (0.5 g) was added to the untreated compound 46B solution from the previous step. The system was purged twice with hydrogen and stirred overnight at room temperature in a hydrogen atmosphere. When LCMS indicated the depletion of the starting materials, the reaction solution was filtered through celite and purified by preparative HPLC to give a yellow solid compound 46 (8.2 mg, 7.5% yield over the two steps). MS-ESI: m/z 478.2 [M+H]⁺.

¹H NMR (400 MHz, DMSO) δ 8.14 (s, 1H), 7.38 (d, J=6.5 Hz, 2H), 7.17 (d, J=20.9 Hz, 2H), 7.06 (s, 1H), 6.87 (d, J=7.4 Hz, 2H), 6.41 (d, J=7.3 Hz, 2H), 4.59 (s, 2H), 4.28 (d, J=37.6 Hz, 2H), 3.59 (dd, J=23.4, 7.9 Hz, 7H), 3.25 (d, J=10.5 Hz, 1H), 2.82 (d, J=14.7 Hz, 1H), 1.88 (d, J=56.6 Hz, 3H), 1.63 (d, J=6.4 Hz, 2H), 0.89 (s, 3H).

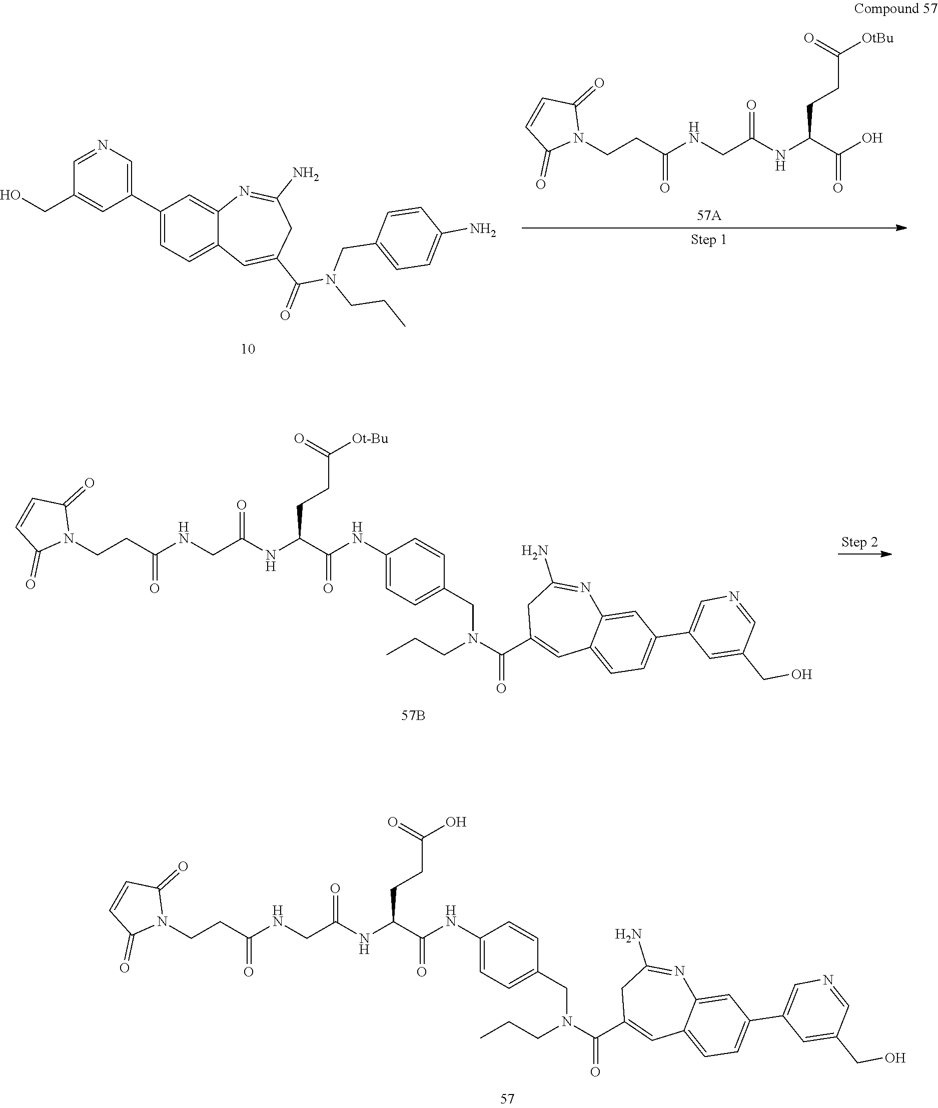 [SMILES: CCCN(Cc1ccc(N)cc1)C(=O)C1=Cc2ccc(-c3cncc(CO)c3)cc2N=C(N)C1 ; CC(C)(C)OC(=O)CC[C@H](NC(=O)CNC(=O)CCN1C(=O)C=CC1=O)C(=O)O ; CCCN(Cc1ccc(NC(=O)[C@H](CCC(=O)OC(C)(C)C)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCN2C(=O)C=CC2=O)cc1)C(=O)C1=Cc2ccc(-c3cncc(CO)c3)cc2N=C(N)C1]

Step 1 Compound 10 (300 mg, 0.609 mmol, 1 eq) and compound 57A (301 mg, 0.731 mmol, 1.2 eq) were added to a mixed solvent of DCM (3 mL) and DMA (3 mL). The system was purged twice with argon before EDCI (468 mg, 2.44 mmol, 4 eq) was added at room temperature, then purged twice again with argon, and stirred at room temperature overnight. When LCMS indicated the formation of the target product, the mixture was directly purified by preparative HPLC to give a white solid 57B (50 mg, 9.7% yield). MS-ESI: m/z 849.4 [M+H]⁺.

Step 2 Compound 57B (50 mg, 0.0589 mmol, 1 eq) was dissolved in DCM (3 mL), before TFA (0.5 mL) was added in an ice-water bath. The system was stirred at room temperature for 2 h. When LCMS indicated the depletion of the starting materials, the mixture was concentrated at reduced pressure and puriied by preparative HPLC to give a white solid 57 (17 mg, 37% yield).

MS-ESI: m/z 793.4 [M+H]⁺.

¹H NMR (400 MHz, DMSO) δ 12.02 (s, 1H), 10.05-9.75 (m, 2H), 9.09 (s, 1H), 8.86 (s, 1H), 8.60 (s, 1H), 8.32-8.30 (t, J=5.6 Hz, 1H), 8.23-8.21 (d, J=7.8 Hz, 1H), 8.10 (s, 1H), 7.85-7.59 (m, 5H), 7.39-7.17 (m, 2H), 7.13 (s, 1H), 7.00 (s, 2H), 4.65 (s, 4H), 4.43-4.34 (dd, J=13.4, 8.1 Hz, 1H), 3.72-3.71 (d, J=5.6 Hz, 2H), 3.63-3.52 (m, 3H), 2.44-2.41 (m, 3H), 2.30-2.22 (m, 2H), 2.06-1.93 (m, 1H), 1.90-1.75 (m, 1H), 1.59-1.54 (dd, J=14.8, 7.4 Hz, 2H), 0.90-0.70 (m, 3H).

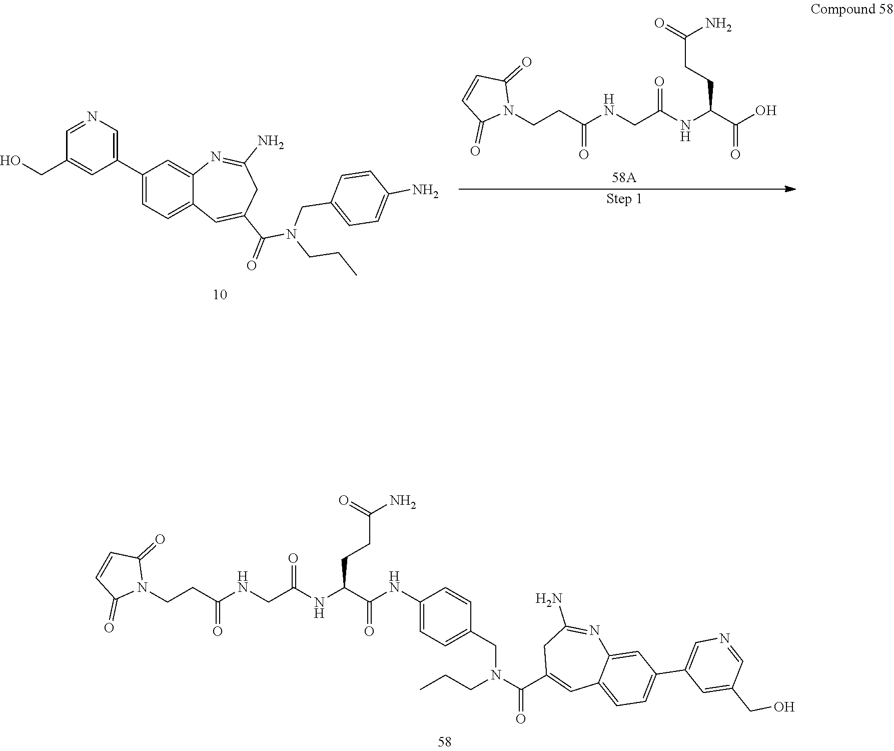 [SMILES: CCCN(Cc1ccc(N)cc1)C(=O)C1=Cc2ccc(-c3cncc(CO)c3)cc2N=C(N)C1 ; NC(=O)CC[C@H](NC(=O)CNC(=O)CCN1C(=O)C=CC1=O)C(=O)O ; CCCN(Cc1ccc(NC(=O)[C@H](CCC(N)=O)NC(=O)CNC(=O)CCN2C(=O)C=CC2=O)cc1)C(=O)C1=Cc2ccc(-c3cncc(CO)c3)cc2N=C(N)C1]

Step 1 Compound 10 (500 mg, 1.02 mmol, 1 eq) and compound 58A (360 mg, 1.02 mmol, 1 eq) were added to a mixed solvent of DCM (10 mL) and DMA (10 mL). The system was purged twice with argon before EDCI (779 mg, 4.06 mmol, 4 eq) was added at room temperature, then purged twice again with argon, and stirred at room temperature overnight. When LCMS indicated the formation of the target product, the mixture was directly purified by preparative HPLC, lyophilized, and then purified again by preparative HPLC to give a white solid 58 (8 mg, 1% yield).

MS-ESI. m/z 792.4 [M+H]⁺.

¹H NMR (400 MHz, DMSO) δ 11.93 (s, 1H), 10.0-9.74 (m, 2H), 9.02 (s, 1H), 8.83 (s, 1H), 8.59 (s, 1H), 8.31-8.28 (t, J=5.6 Hz, 1H), 8.25-8.23 (d, J=7.7 Hz, 1H), 8.06 (s, 1H), 7.81-7.69 (m, 3H), 7.65-7.63 (d, J=8.6 Hz, 2H), 7.33-7.19 (m, 3H), 7.13 (s, 1H), 6.99 (s, 2H), 6.78 (s, 1H), 4.74-4.57 (m, 4H), 4.37-4.32 (dd, J=13.5, 7.8 Hz, 1H), 3.71-3.70 (d, J=5.6 Hz, 2H), 3.65-3.59 (m, 2H), 3.04 (s, 2H), 2.79-2.78 (d, J=3.8 Hz, 2H), 2.46-2.41 (m, 2H), 2.19-2.10 (m, 2H), 2.01-1.94 (m, 1H), 1.86-1.79 (m, 1H), 1.59-1.54 (dd, J=14.9, 7.5 Hz, 2H), 0.87-0.74 (m, 3H).

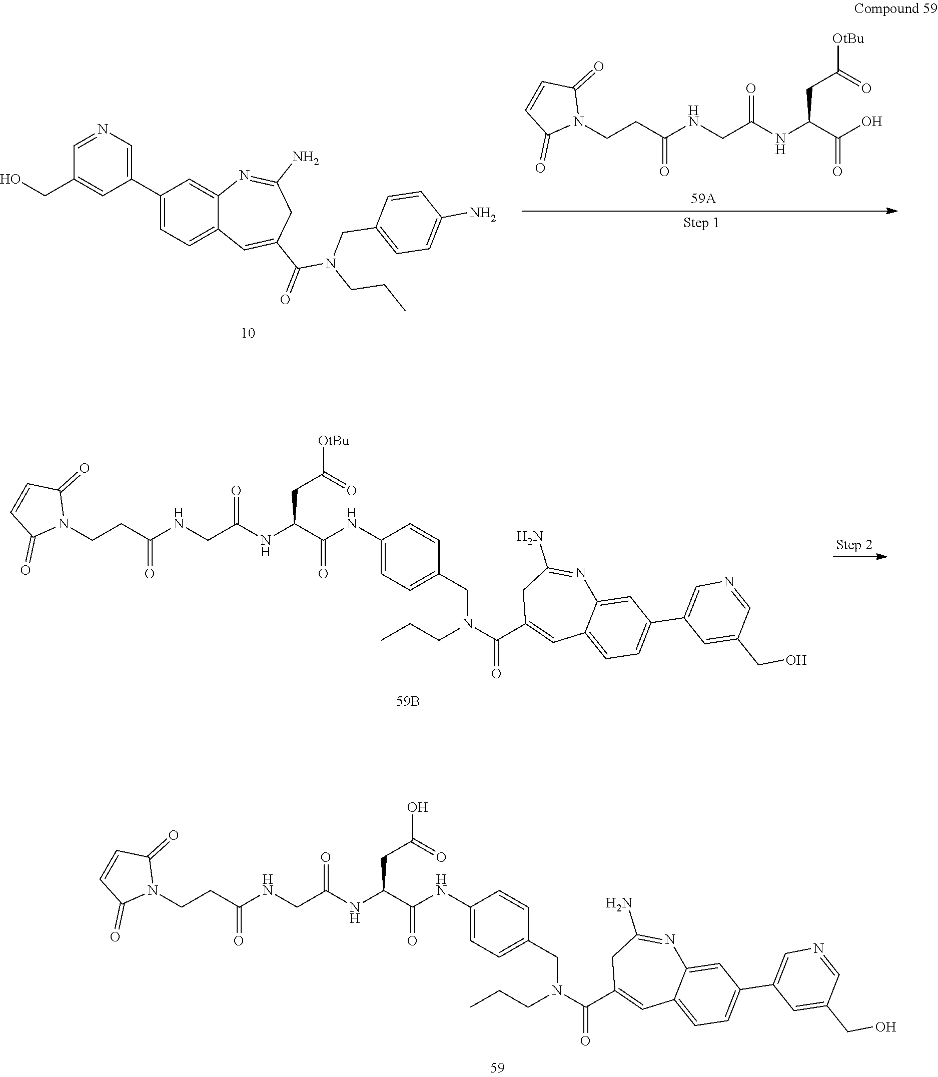 [SMILES: CCCN(Cc1ccc(N)cc1)C(=O)C1=Cc2ccc(-c3cncc(CO)c3)cc2N=C(N)C1 ; CC(C)(C)OC(=O)C[C@H](NC(=O)CNC(=O)CCN1C(=O)C=CC1=O)C(=O)O ; CCCN(Cc1ccc(NC(=O)[C@H](CC(=O)OC(C)(C)C)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CC(=O)O)NC(=O)CNC(=O)CCN2C(=O)C=CC2=O)cc1)C(=O)C1=Cc2ccc(-c3cncc(CO)c3)cc2N=C(N)C1]

Step 1 Compound 10 (400 mg, 0.813 mmol, 1 eq) and compound 59A (420 mg, 1.06 mmol, 1.3 eq) were added to DMF (8 mL), before DIEA (525.4 mg, 4.06 mmol, 5 eq) and T3P (1.55 g, 2.44 mmol, 3 eq, 50%) were added. The system was stirred at room temperature overnight. When LCMS indicated the formation of the product, the mixture was directly purified by preparative HPLC to give a white solid 59B (50 mg, 7.4% yield). MS-ESI: m/z 835.3 [M+H]⁺.

Step 2 Compound 59B (50 mg, 0.0599 mmol, 1 eq) was dissolved in DCM (3 mL), before TFA (0.5 mL) was added in an ice-water bath. The system was stirred at room temperature for 2 h. When LCMS indicated the depletion of the starting materials, the mixture was directly purified by preparative HPLC to give a white solid 59 (15 mg, 33% yield).

MS-ESI: m/z 779.4 [M+H]⁺.

¹H NMR (400 MHz, DMSO) δ 12.01 (s, 1H), 10.01-9.64 (m, 2H), 9.08 (s, 1H), 8.86 (s, 1H), 8.60 (s, 1H), 8.45-8.26 (m, 2H), 8.09 (s, 1H), 7.88-7.63 (m, 5H), 7.51-7.15 (m, 3H), 7.13 (s, 1H), 7.00 (s, 2H), 4.83-4.51 (m, 6H), 3.76-3.67 (dd, J=15.3, 9.8 Hz, 2H), 3.64-3.61 (t, J=7.4 Hz, 3H), 2.80-2.75 (dd, J=16.7, 5.9 Hz, 1H), 2.63-2.57 (dd, J=16.7, 8.0 Hz, 1H), 2.44-2.42 (d, J=6.5 Hz, 2H), 1.59-1.54 (dd, J=14.7, 7.7 Hz, 2H), 0.80 (s, 3H).

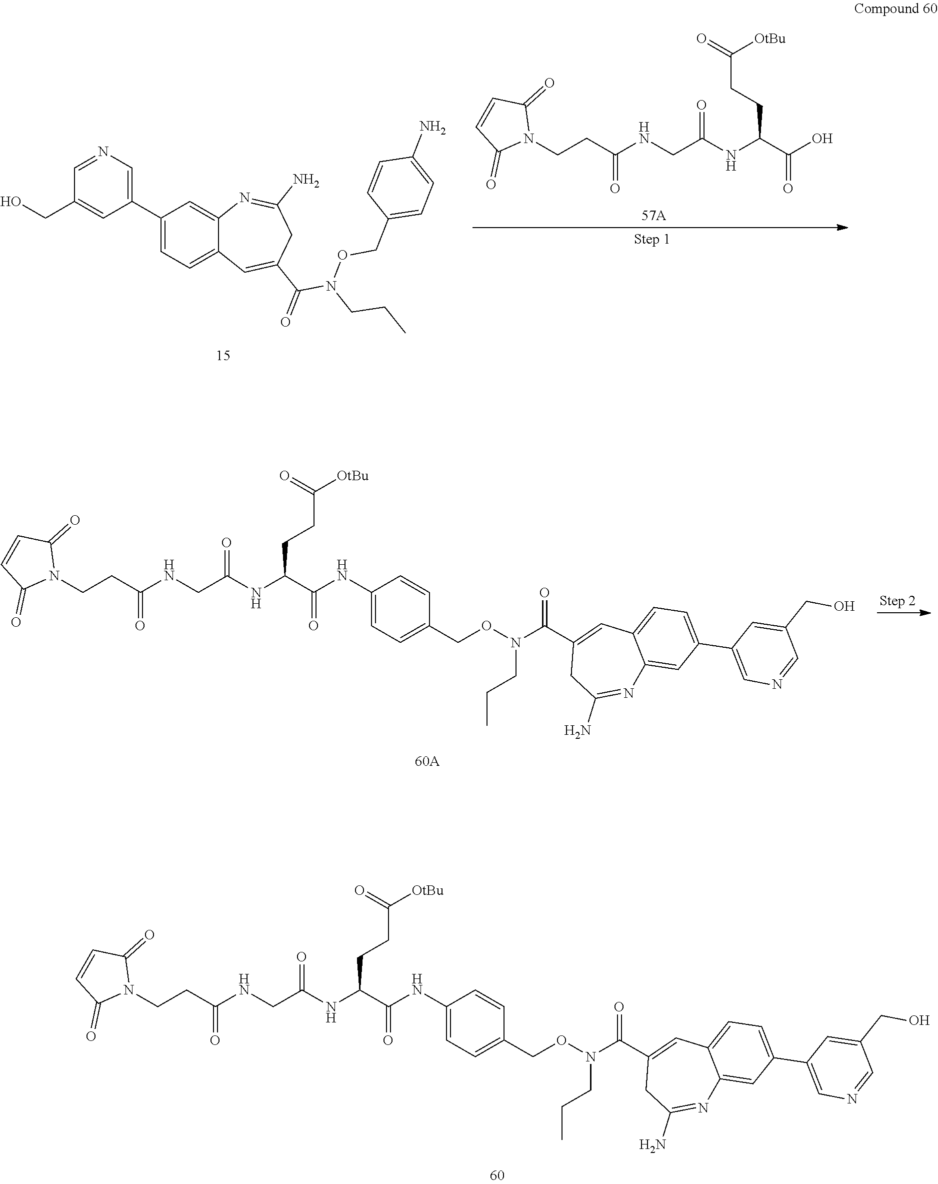 [SMILES: CCCN(OCc1ccc(N)cc1)C(=O)C1=Cc2ccc(-c3cncc(CO)c3)cc2N=C(N)C1 ; CC(C)(C)OC(=O)CC[C@H](NC(=O)CNC(=O)CCN1C(=O)C=CC1=O)C(=O)O ; CCCN(OCc1ccc(NC(=O)[C@H](CCC(=O)OC(C)(C)C)NC(=O)CNC(=O)CCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(=O)OC(C)(C)C)NC(=O)CNC(=O)CCN2C(=O)C=CC2=O)cc1)C(=O)C1=Cc2ccc(-c3cncc(CO)c3)cc2N=C(N)C1]

Step 1 Compound 15 (450 mg, 0.954 mmol, 1 eq) and compound 57A (511 mg, 1.24 mmol, 1.3 eq) were added to a mixed solvent of DCM (5 mL) and DMA (5 mL). The system was purged twice with argon before EDCI (732 mg, 3.82 mmol, 4 eq) was added at room temperature, then purged twice again with argon, and stirred at room temperature overnight. When LCMS indicated the formation of the product, the mixture was directly purified by preparative HPLC to give a white solid 60A (25 mg, 3% o yield). MS-ESI: m/z 865.4 [M+H]⁺.

Step 2 Compound 60A (25 mg, 0.0289 mmol, 1 eq) was dissolved in DCM (3 mL), before TFA (0.5 mL) was added in an ice-water bath. The system was stirred at room temperature (25° C.) for 2 h.

When LCMS indicated the depletion of the starting materials, the mixture was concentrated at reduced pressure and purified by preparative HPLC to give a white solid 60 (7 mg, 30% yield).

MS-ESI: m/z 809.3 [M+H]⁺.

¹H NMR (400 MHz, DMSO) δ 11.91 (s, 1H), 9.92 (s, 1H), 9.83 (s, 1H), 8.93 (s, 1H), 8.87-8.85 (d, J=2.1 Hz, 1H), 8.61-8.59 (d, J=1.7 Hz, 1H), 8.28-8.25 (t, J=5.7 Hz, 1H), 8.19-8.17 (d, J=7.8 Hz, 1H), 8.08 (s, 1H), 7.80-7.76 (dd, J=8.2, 1.7 Hz, 1H), 7.73 (s, 1H), 7.65-7.63 (d, J=8.3 Hz, 1H), 7.57-7.55 (d, J=8.5 Hz, 2H), 7.28-7.15 (m, 3H), 6.97 (s, 2H), 4.81 (s, 2H), 4.65 (s, 3H), 3.72-3.68 (dd, J=14.3, 6.4 Hz, 4H), 3.62-3.56 (m, 2H), 2.42-2.38 (m, 2H), 2.30-2.22 (m, 2H), 2.03-1.93 (m, 1H), 1.85-1.75 (m, 1H), 1.74-1.64 (m, 2H), 0.94-0.92 (t, J=7.4 Hz, 3H).

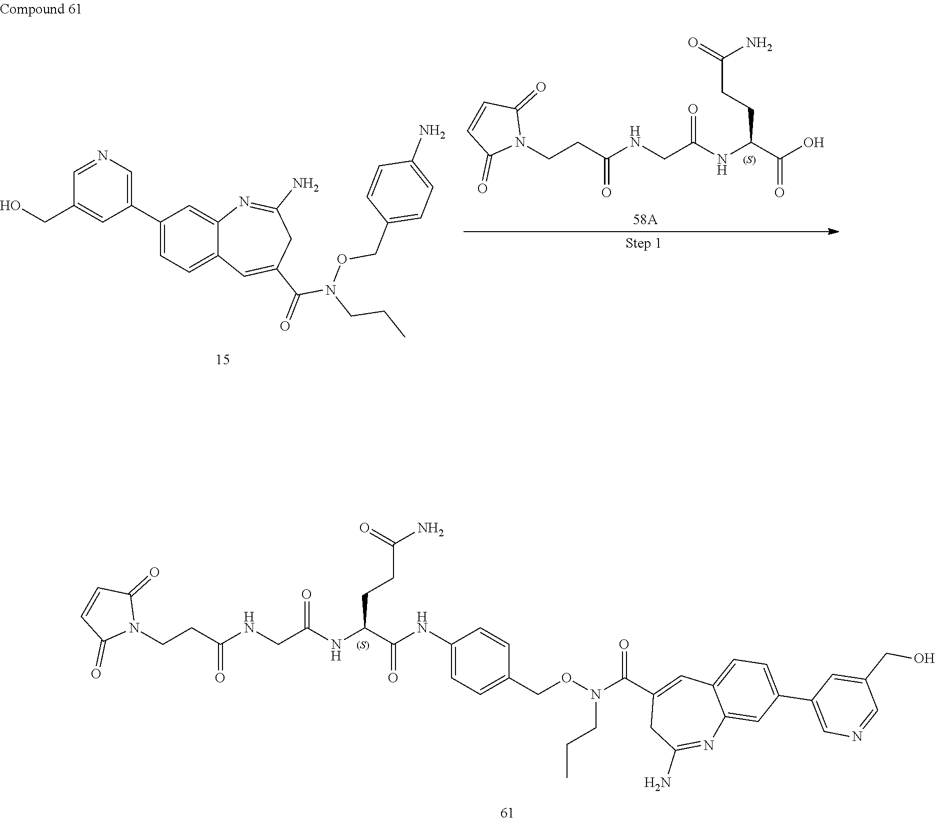

Step 1 Compound 15 (250 mg, 0.53 mmol, 1 eq) and compound 58A (188 mg, 0.53 mmol, 1 eq) were added to a mixed solvent of DCM (10 mL) and DMA (10 mL). The system was purged twice with argon before EDCI (407 mg, 2.12 mmol, 4 eq) was added at room temperature, then purged twice again with argon, and stirred at room temperature overnight. When LCMS indicated the formation of the product, the mixture was directly purified by preparative HPLC to give a white solid 61 (20 mg, 4.7% yield).

MS-ESI: m/z 808.4 [M+H]⁺.

¹H NMR (400 MHz, DMSO) δ 12.24 (s, 1H), 9.96 (s, 1H), 9.87 (s, 1H), 9.25 (s, 1H), 8.92-8.91 (d, J=2.0 Hz, 1H), 8.65 (s, 1H), 8.33-8.31 (t, J=5.6 Hz, 1H), 8.27-8.26 (d, J=7.6 Hz, 1H), 8.18 (s, 1H), 7.82-7.80 (d, J=8.2 Hz, 1H), 7.75 (s, 1H), 7.67-7.65 (d, J=8.2 Hz, 1H), 7.59-7.57 (d, J=8.4 Hz, 2H), 7.33 (s, 1H), 7.26-7.24 (d, J=8.5 Hz, 2H), 7.21 (s, 1H), 7.00 (s, 2H), 6.81 (s, 1H), 4.83 (s, 2H), 4.69 (s, 2H), 4.36-4.31 (m, 1H), 3.76-3.70 (dd, J=16.3, 6.2 Hz, 5H), 3.63 (s, 1H), 3.32 (s, 2H), 2.47-2.40 (m, 2H), 2.24-2.09 (m, 2H), 2.04-1.91 (m, 1H), 1.89-1.79 (m, 1H), 1.73-1.68 (dd, J=14.4, 7.2 Hz, 2H), 0.97-0.93 (t, J=7.4 Hz, 3H).

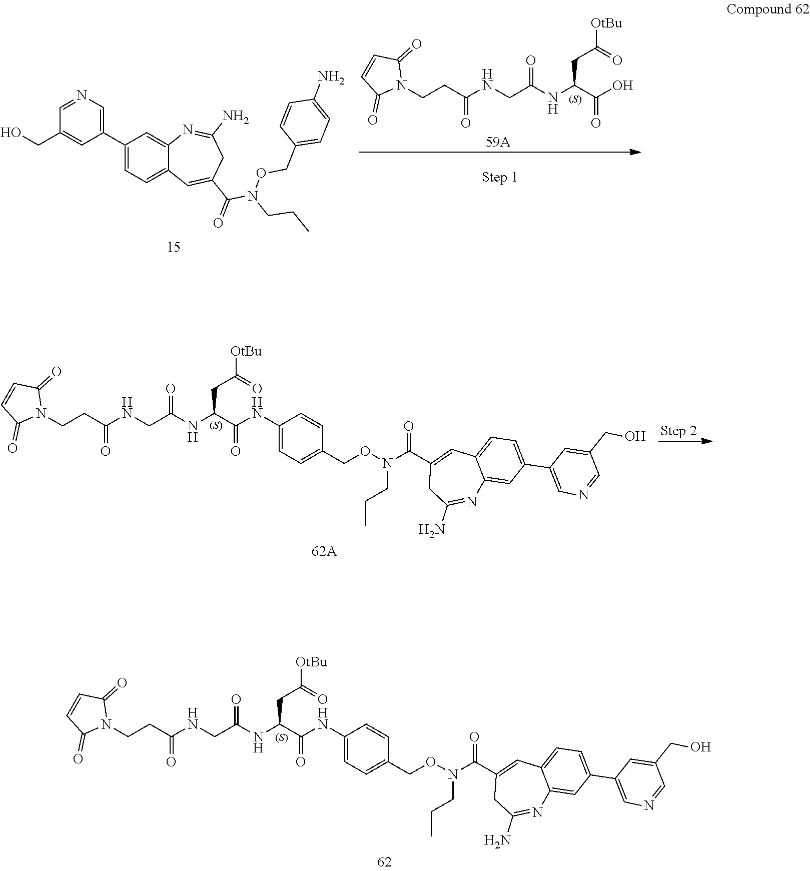

Step 1 Compound 59A (375.5 mg, 0.945 mmol, 1 eq) was added to DMF (40 mL), before NMI (258.6 mg, 258.6 mg, 4 eq) and TCFH (331.4 mg, 1.18 mmol, 1.5 eq) were added at room temperature. The system was stirred at room temperature for 3 min, before a hydrochloride of compound 15 (400 mg, 0.787 mmol, 1 eq) was added. The mixture was then purged once with argon and stirred at room temperature overnight. When LCMS indicated the formation of the product, the mixture was directly purified by preparative HPLC to give a yellow solid 62A (1.1 g, crude product, yield not calculated). MS-ESI: m/z 851.4 [M+H]⁺.

Step 2 Compound 62A (1.1 g, 1.29 mmol, 1 eq) was dissolved in DCM (50 mL), before TFA (10 mL) was added in an ice-water bath. The system was stirred at room temperature for 2 h. When LCMS indicated the depletion of the starting materials, the mixture was concentrated at 0° C. and reduced pressure to remove most of the DCM and purified by preparative HPLC to give a pale yellow solid 62 (240 mg, 7.6% yield over the two steps).

MS-ESI: m/z 795.3 [M+H]⁺.

¹H NMR (400 MHz, DMSO) δ 12.32 (s, 1H), 9.88-9.81 (d, J=25.1 Hz, 2H), 9.36 (s, 1H), 8.91 (s, 1H), 8.65 (s, 1H), 8.39-8.36 (t, J=7.2 Hz, 2H), 8.17 (s, 1H), 7.88-7.78 (m, 1H), 7.75 (s, 1H), 7.68-7.61 (dd, J=19.8, 8.4 Hz, 3H), 7.30-7.22 (m, 3H), 7.00 (s, 2H), 4.84 (s, 2H), 4.74-4.68 (m, 3H), 3.76-3.71 (dd, J=12.3, 5.0 Hz, 3H), 3.64-3.56 (m, 2H), 3.33 (s, 2H), 2.80-2.74 (dd, J=16.4, 5.8 Hz, 1H), 2.63-2.57 (dd, J=16.5, 7.8 Hz, 1H), 2.48-2.27 (m, 2H), 1.74-1.68 (dd, J=14.4, 7.1 Hz, 2H), 0.97-0.93 (t, J=7.4 Hz, 3H).

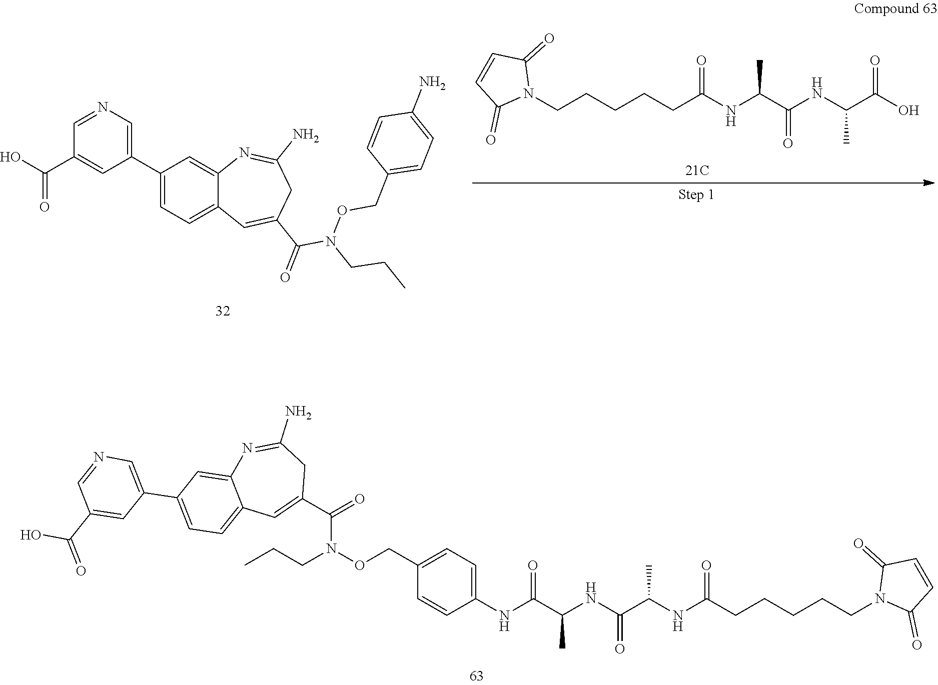 [SMILES: CCCN(OCc1ccc(N)cc1)C(=O)C1=Cc2ccc(-c3cncc(C(=O)O)c3)cc2N=C(N)C1 ; C[C@H](NC(=O)[C@H](C)NC(=O)CCCCCN1C(=O)C=CC1=O)C(=O)O ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(C(=O)O)c3)cc2N=C(N)C1]

Step 1 Compound 21C (162.5 mg, 0.46 mmol, 1.2 eq) was added to DMF (20 mL), before NMI (126 mg, 1.53 mmol, 4 eq) and TCFH (161.3 mg, 0.575 mmol, 1.5 eq) were added. The system was stirred at room temperature for 2 min, before a hydrochloride of crude compound 32 (200 mg, 0.383 mmol, 1 eq) was added. The mixture was then stirred at room temperature overnight. When LCMS indicated the depletion of the starting materials and the formation of the target product, the mixture was directly purified by preparative HPLC to give a white solid 63 (20 mg, 6.4% yield).

MS-ESI. m/z 821.4 [M+H]⁺.

¹H NMR (400 MHz, DMSO) δ 11.96 (s, 1H), 9.87-9.85 (d, J=6.7 Hz, 2H), 9.20 (d, J=2.2 Hz, 1H), 9.13 (d, J=1.8 Hz, 1H), 9.02 (s, 1H), 8.55-8.54 (t, J=2.0 Hz, 1H), 8.07-8.05 (d, J=7.2 Hz, 1H), 7.99-7.98 (d, J=6.9 Hz, 1H), 7.88-7.85 (dd, J=8.1, 1.5 Hz, 1H), 7.80 (s, 1H), 7.66-7.64 (d, J=8.3 Hz, 1H), 7.56-7.54 (d, J=8.5 Hz, 2H), 7.24-7.21 (d, J=9.5 Hz, 3H), 6.99 (s, 2H), 4.81 (s, 2H), 4.38-4.31 (p, J=6.9 Hz, 1H), 4.26-4.19 (p, J=7.0 Hz, 1H), 3.74-3.71 (t, J=6.9 Hz, 2H), 3.36-3.33 (d, J=7.1 Hz, 2H), 3.30 (s, 2H), 2.11-2.07 (t, J=7.3 Hz, 2H), 1.76-1.63 (m, 2H), 1.48-1.41 (dt, J=14.6, 7.3 Hz, 4H), 1.29-1.27 (d, J=7.1 Hz, 4H), 1.22-1.11 (m, 5H), 0.95-0.92 (t, J=7.4 Hz, 3H).

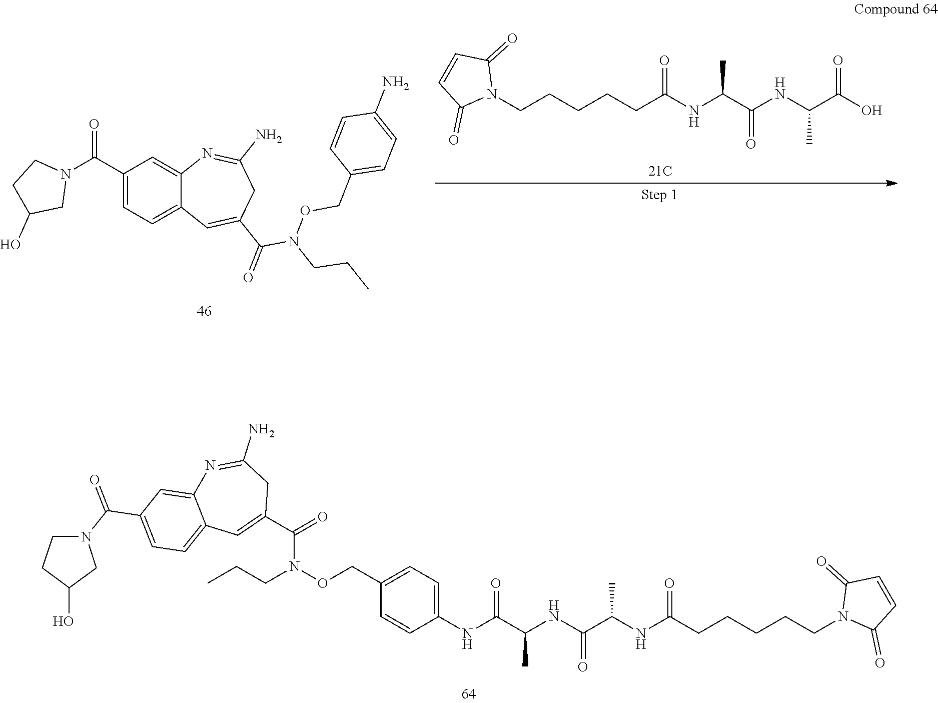 [SMILES: CCCN(OCc1ccc(N)cc1)C(=O)C1=Cc2ccc(C(=O)N3CCC(O)C3)cc2N=C(N)C1 ; C[C@H](NC(=O)[C@H](C)NC(=O)CCCCCN1C(=O)C=CC1=O)C(=O)O ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N3CCC(O)C3)cc2N=C(N)C1]

Step 1 Compound 21C (123.7 mg, 0.35 mmol, 1.2 eq) was added to DMF (5 mL), before NMI (96 mg, 1.17 mmol, 4 eq) and TCFH (123 mg, 0.438 mmol, 1.5 eq) were added. The system was stirred at room temperature for 2 min, before a hydrochloride of crude compound 46 (150 mg, 0.292 mmol, 1 eq) was added. The mixture was then stirred at room temperature overnight. When LCMS indicated the depletion of the starting materials and the formation of the target product, the mixture was purified by preparative HPLC to give a white solid 64 (110 mg, 43% yield).

MS-ESI: m/z 813.4 [M+H]⁺.

¹H NMR (400 MHz, DMSO) δ 12.14 (s, 1H), 9.80-9.88 (d, J=6.1 Hz, 1H), 9.83 (s, 1H), 9.19-9.18 (d, J=6.3 Hz, 1H), 8.07-8.06 (d, J=7.2 Hz, 1H), 8.0-7.98 (d, J=7.0 Hz, 1H), 7.55-7.49 (m, 5H), 7.22-7.20 (d, J=7.2 Hz, 2H), 7.15 (d, J=0.7 Hz, 1H), 6.99 (s, 2H), 4.79 (s, 2H), 4.37-4.18 (m, 3H), 3.72-3.68 (t, J=7.1 Hz, 2H), 3.65-3.56 (m, 3H), 3.39-3.34 (dd, J=14.3, 7.3 Hz, 3H), 3.31-3.19 (m, 3H), 2.12-2.08 (t, J=7.4 Hz, 2H), 2.03-1.80 (m, 2H), 1.73-1.62 (m, 2H), 1.51-1.42 (dt, J=14.2, 7.1 Hz, 4H), 1.29-1.28 (d, J=7.1 Hz, 3H), 1.22-1.11 (m, 5H), 0.94-0.90 (t, J=7.4 Hz, 3H).

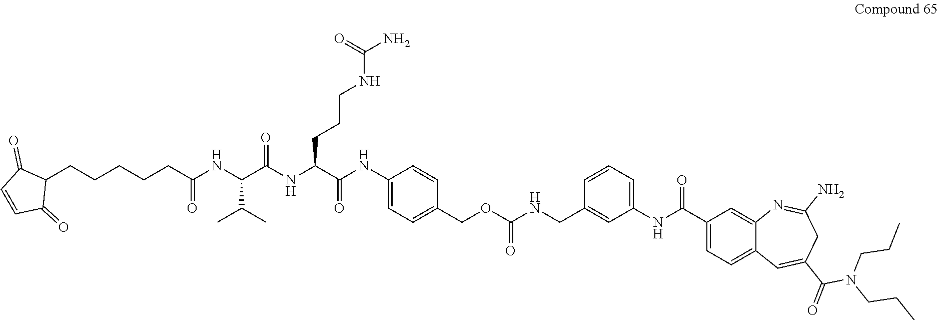 [SMILES: CCCN(CCC)C(=O)C1=Cc2ccc(C(=O)Nc3cccc(CNC(=O)OCc4ccc(NC(=O)[C@H](CCCNC(N)=O)NC(=O)[C@@H](NC(=O)CCCCCC5C(=O)C=CC5=O)C(C)C)cc4)c3)cc2N=C(N)C1]

It was synthesized with reference to the patent WO2020056194A1.

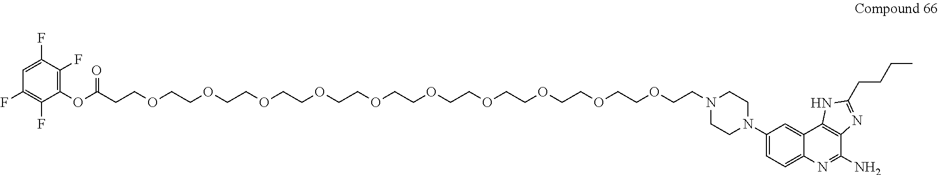 [SMILES: CCCCc1nc2c(N)nc3ccc(N4CCN(CCOCCOCCOCCOCCOCCOCCOCCOCCOCCOCCC(=O)Oc5c(F)c(F)cc(F)c5F)CC4)cc3c2[nH]1]

It was synthesized with reference to the patent WO2020190725A1.

### Example 2 Preparation of Ligand-Drug Conjugates

### 2.1 Preparation of Antibodies

The following antibodies could be prepared according to the conventional methods for antibodies. For example, vectors could be constructed for transfecting eukaryotic cells such as HEK293 cells (Life Technologies, Cat No. 11625019), followed by purification of expressed proteins.

### Sequences of Trastuzumab:

Light chain
(SEQ ID NO: 33)
DIQMTQSPSSLSASVGDRVTITCRASQDVNTAVAWYQQKPGKAPKLLIYSASFLYSGVPSRF
SGSRSGTDFTLTISSLQPEDFATYYCQQHYTTPPTFGQGTKVEIKRTVAAPSVFIFPPSDEQLK
SGTASVVCLLNNFYPREAKVQWKVDNALQSGNSQESVTEQDSKDSTYSLSSTLTLSKADY
EKHKVYACEVTHQGLSSPVTKSFNRGEC
Heavy chain
(SEQ ID NO: 37)
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSPG
Sequences of antibody pertuzumab
Light chain
(SEQ ID NO: 34)
DIQMTQSPSSLSASVGDRVTITCKASQDVSIGVAWYQQKPGKAPKLLIYSASYRYTGVPSRF
SGSGSGTDFTLTISSLQPEDFATYYCQQYYIYPYTFGQGTKVEIKRTVAAPSVFIFPPSDEQLK
SGTASVVCLLNNFYPREAKVQWKVDNALQSGNSQESVTEQDSKDSTYSLSSTLTLSKADY
EKHKVYACEVTHQGLSSPVTKSFNRGEC
Heavy chain
(SEQ ID NO: 38)
EVQLVESGGGLVQPGGSLRLSCAASGFTFTDYTMDWVRQAPGKGLEWVADVNPNSGGSIY
NQRFKGRFTLSVDRSKNTLYLQMNSLRAEDTAVYYCARNLGPSFYFDYWGQGTLVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPG
Sequences of hRS7 antibody (sacituzumab)
Light chain
(SEQ ID NO: 35)
DIQLTQSPSSLSASVGDRVSITCKASQDVSIAVAWYQQKPGKAPKLLIYSASYRYTGVPDRFS
GSGSGTDFTLTISSLQPEDFAVYYCQQHYITPLTFGAGTKVEIKRTVAAPSVFIFPPSDEQLKS
GTASVVCLLNNFYPREAKVQWKVDNALQSGNSQESVTEQDSKDSTYSLSSTLTLSKADYE
KHKVYACEVTHQGLSSPVTKSFNRGEC
Heavy chain
(SEQ ID NO: 39)
QVQLQQSGSELKKPGASVKVSCKASGYTFTNYGMNWVKQAPGQGLKWMGWINTYTGEP
TYTDDFKGRFAFSLDTSVSTAYLQISSLKADDTAVYFCARGGFGSSYWYFDVWGQGSLVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGN
VFSCSVMHEALHNHYTQKSLSLSPGK
Sequence of antibody codrituzumab
Light chain
(SEQ ID NO: 36)
DVVMTQSPLSLPVTPGEPASISCRSSQSLVHSNRNTYLHWYLQKPGQSPQLLIYKVSNRFSG
VPDRFSGSGSGTDFTLKISRVEAEDVGVYYCSQNTHVPPTFGQGTKLEIKRTVAAPSVFIFPP
SDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Heavy chain
(SEQ ID NO: 40)
QVQLVQSGAEVKKPGASVKVSCKASGYTFTDYEMHWVRQAPGQGLEWMGALDPKTGDT
AYSQKFKGRVTLTADKSTSTAYMELSSLTSEDTAVYYCTRFYSYTYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVM
HEALHNHYTQKSLSLSPGK

### Example 2.2 Preparation of Ligand-Drug Conjugates

In the present application, a 20 mM aqueous succinate buffer solution at pH 5.0 was obtained by adjusting the pH of a 20 mM aqueous succinic acid solution to 5.0 with NaH, and a 20 mM aqueous histidine buffer solution at pH 5.5 was obtained by adjusting the pH of a 20 mM aqueous histidine solution to 5.5 with HOAc.

The protein purity of the ADCs in the present application was detected by the SEC method, and the parameters are shown below.

| Instrument | Agilent Technologies 1260 | |
|---|---|---|
| Chromatography | TOSON - G3000SWXL, 300*7.8 mm, 5 μm | |
| column | | |
| Column temperature | 22° C. | |
| Wavelength | 280 nm | |
| Injection volume | 30~50 μg | |
| Mobile phase | 0.2M K₂HPO₄/KH₂PO₄, 0.25M KCl, 15%(v/v) | |
| | 2-propanol, pH 7.0 | |
| | Time (min) | Flow rate (mL/min) |
| Gradient | 0.0 | 0.75 |
| | 18.0 | 0.75 |

The DAR values of the ADCs in the present application were detected by the LC-MS method. The experimental procedure was as follows: To 40.0 μg of an ADC sample solution were added 75.0 μL of 8.0 mol/L guanidine hydrochloride (Gdn-HCl), 5.0 μL of 1.0 mol/L Tris-HCl, and 2.0 μL of 1 mol/L DTT. The solution was diluted to 100.0 μL with ultrapure water, mixed well, and incubated at 22° C. for 30 min. Drug/antibody ratio (DAR) was then analyzed using LC-MS. The LC parameters are shown below.

| Instrument | Agilent Technologies 1260 | | | |
|---|---|---|---|---|
| Reversed-phase | Agilent, PLRP-S 2.1*50 mm, 8 μm | | | |
| chromatography | | | | |
| column | | | | |
| Column temperature | 80° C. | | | |
| Sample temperature | 2~8° C. | | | |
| Detection wavelength | 280 nm | | | |
| Injection volume | 10.0 μL | | | |
| Mobile phase | Mobile phase A: 0.025% TFA and 0.1% | | | |
| | FA in ultrapure water | | | |
| | Mobile phase B: 0.025% TFA and 0.1% | | | |
| | FA in ACN | | | |
| | Time (min) | % A | % B | Flow rate (mL/min) |
| Gradient | 0.0 | 75 | 25 | 0.5 |
| | 0.0 | 75 | 25 | |
| | 0.7 | 66 | 34 | |
| | 5.0 | 55 | 45 | |
| | 6.0 | 10 | 90 | |
| | 7.0 | 10 | 90 | |
| | 7.1 | 75 | 25 | |
| | 10.0 | 75 | 25 | |

The MS parameters are shown below.

| Instrument | Agilent Technologies 6224 TOF LC/MS | |
|---|---|---|
| Ionization mode | Positive | |
| Gas temperature | 350° | C. |
| Air flow | 13 | L/min |
| Atomizer pressure | 45 | psig |
| Capillary voltage | 5000 | V |
| Crushing voltage | 250 | V |
| Voltage of taper hole | 65 | V |
| Peak-to-peak value of radio | 750 | V |
| frequency voltage on octupole | | |
| Acquisition mode | MS (seg) | |
| Mass range | 500-8000 | m/z |
| Acquisition rate | 1 | spectra/s |
| Acquisition time | 1.4-7.0 | min |

### Preparation Example ADC-3

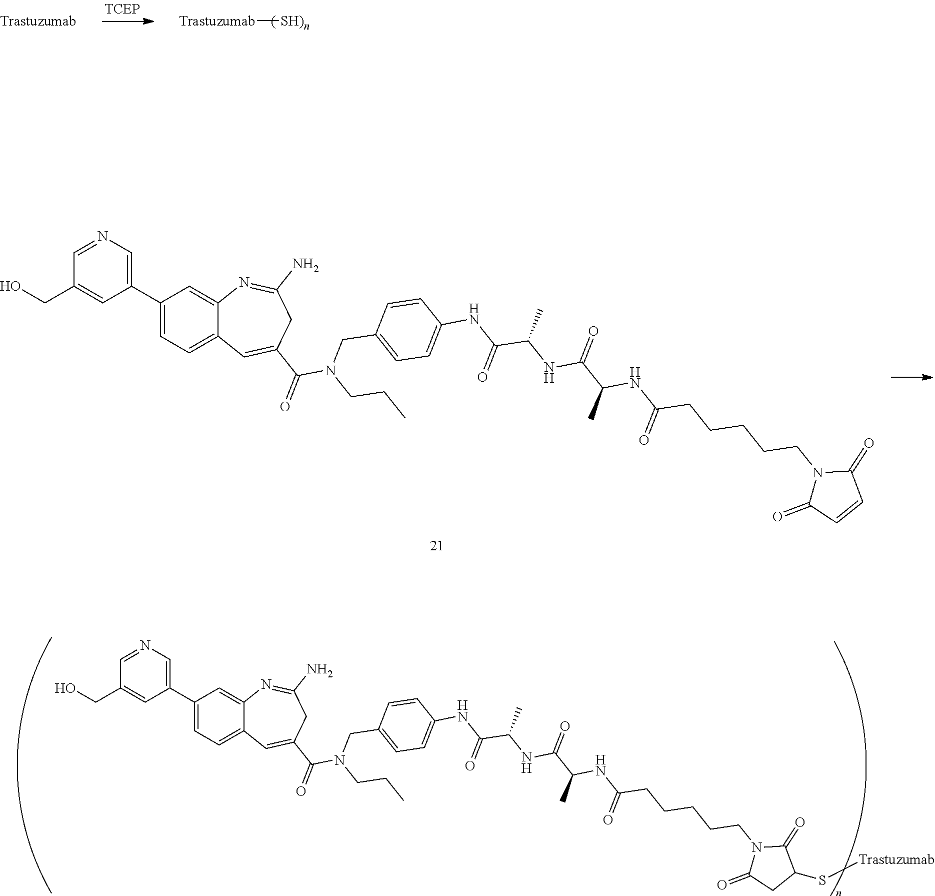

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 32 mg, 10 mg/mL, 0.220 μmol) was added a prepared aqueous solution of tris(2-carboxyethyl)phosphine (5 mM, 0.158 mL, 0.792 μmol) at 22° C. The mixture was placed in a water bath shaker and shaken for reaction at 22° C. for 3 h, and then the reaction was terminated.

21 (2.3 mg, 2.860 μmol) was dissolved in 0.23 mL of DMSO, and the resulting solution was added to the above solution. The mixture was placed in the water bath shaker and shaken for reaction at 22° C. for 2 h. A solution of 21 (1.0 mg, 1.320 μmol) in 0.10 mL of DMSO was added, and the mixture was shaken for reaction at 22° C. for 15 h. A solution of 21 (1.0 mg, 1.320 μmol) in 0.10 mL of DMSO was added, and the mixture was shaken for reaction at 22° C. for 2 h. A solution of 21 (0.7 mg, 0.880 μmol) in 0.070 mL of DMSO was added, the mixture was shaken for reaction at 22° C. for 1 h, and then the reaction was terminated. The reaction solution was desalted and purified by a Sephadex G25 gel column (eluent: 20 mM aqueous histidine buffer solution at pH 5.5) to give a solution of the exemplary product ADC-3 (20 mM aqueous histidine buffer solution at pH 5.5; 19.5 mg, 4.43 mg/mL, 60.9% yield), which was then stored at 4° C.

LC-MS analysis was performed, and the DAR value was calculated to be n=3.63.

### Preparation Example ADC-4

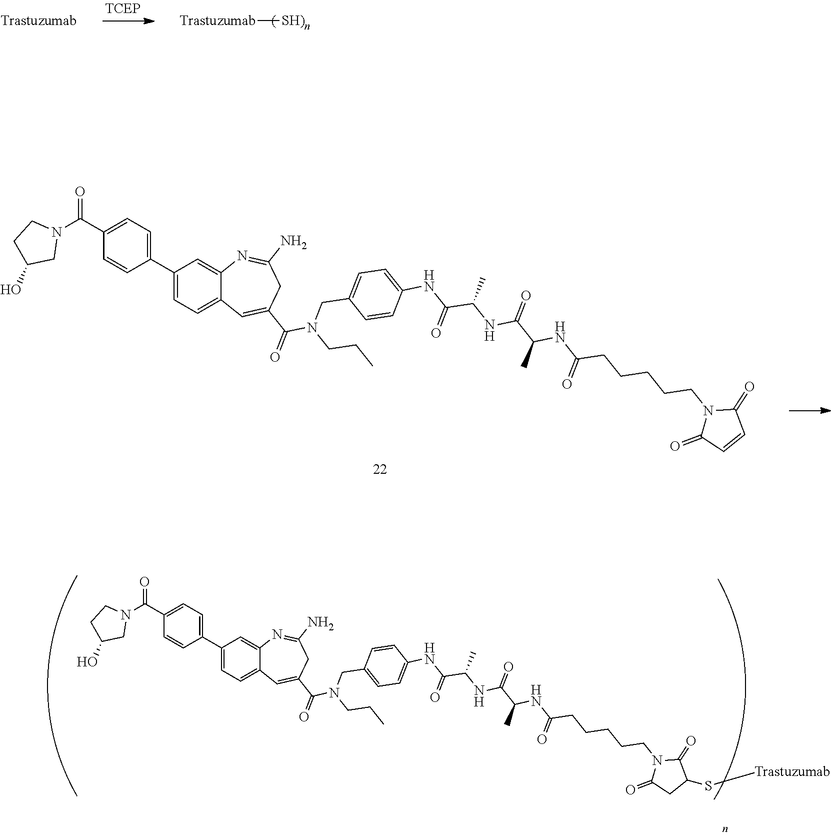

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 21 mg, 10 mg/mL, 0.144 μmol) was added a prepared aqueous solution of tris(2-carboxyethyl)phosphine (5 mM, 0.104 mL, 0.518 μmol) at 22° C. The mixture was placed in a water bath shaker and shaken for reaction at 22° C. for 3 h, and then the reaction was terminated.

22 (1.9 mg, 2.160 μmol) was dissolved in 0.19 mL of DMSO, and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-4 (20 mM aqueous histidine buffer solution at pH 5.5; 14 mg, 5.62 mg/mL, 66.7% yield), which was then stored at 4° C.

LC-MS analysis was performed, and the DAR value was calculated to be n=3.74.

### Preparation Example ADC-5

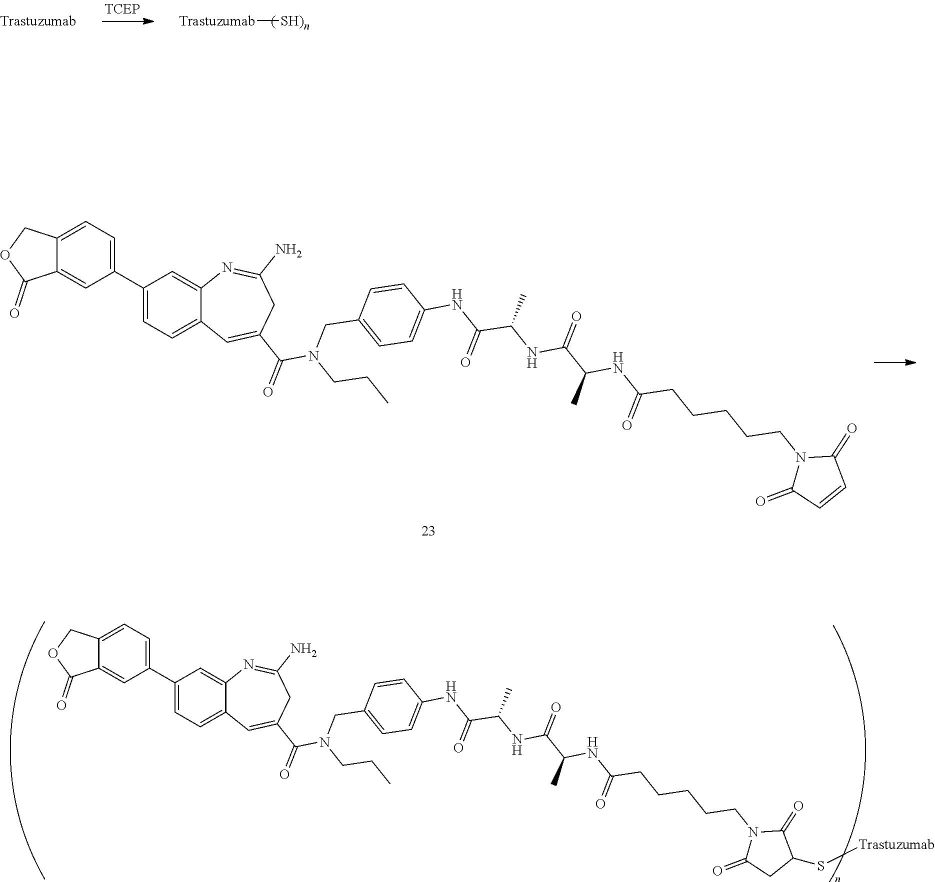

To an aqueous buffer solution of antibody trastuzumab (buffer consisting of 1.65 g/L succinic acid, 1.02 g/L NaOH and 6.30 g/L NaCl at pH 6. 0, containing 2 mM EDTA; 25 mg, 10 mg/mL, 0.172 μmol) was added a prepared aqueous solution of tris(2-carboxyethyl)phosphine (5 mM, 0.124 mL, 0.619 μmol) at 22° C. The mixture was placed in a water bath shaker and shaken for reaction at 22° C. for 3 h, and then the reaction was terminated.

23 (2.1 mg, 2.58 μmol) was dissolved in 0.21 mL of DMSO, and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-5 (20 mM aqueous histidine buffer solution at pH 5.5; 20 mg, 6.76 mg/mL, 80.0% yield), which was then stored at 4° C.

LC-MS analysis was performed, and the DAR value was calculated to be n=3.71.

### Preparation Example ADC-6

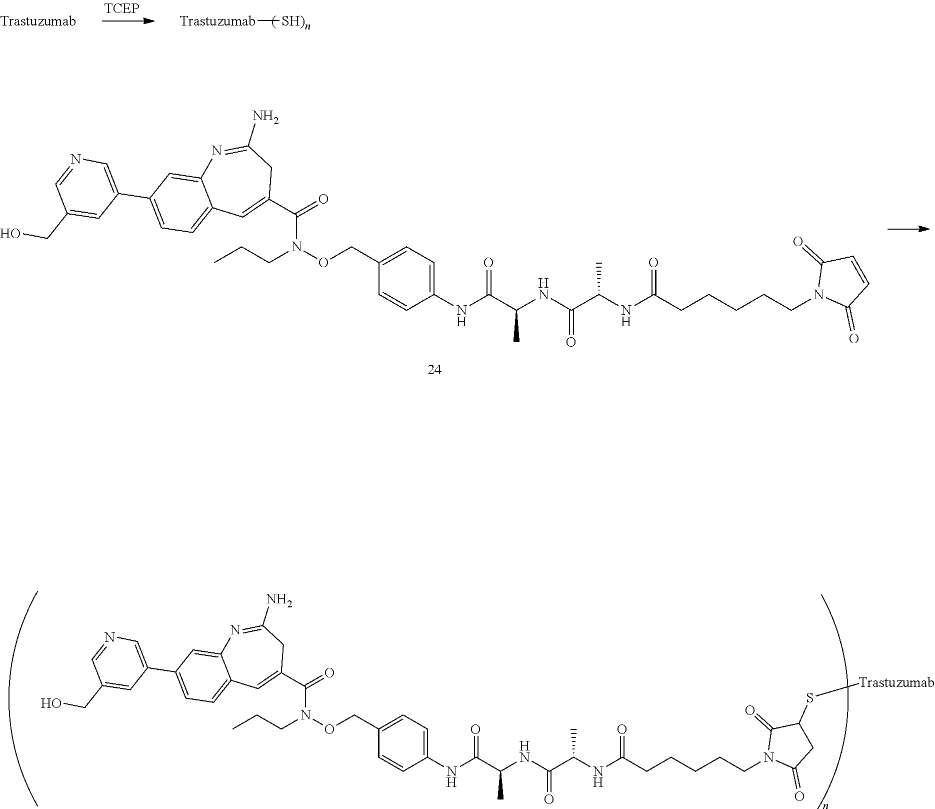

To an aqueous buffer solution of antibody trastuzumab (PB buffer at pH 7.0; 20 mg, 6.25 mg/mL, 0.137 μmol) was added a prepared aqueous solution of tris(2-carboxyethyl)phosphine (5 mM, 0.071 mL, 0.356 μmol) at 37° C. The mixture was placed in a water bath shaker and shaken for reaction at 37° C. for 2 h, and then the reaction was terminated.

24 (0.84 mg, 1.04 μmol) was dissolved in 0.084 mL of DMSO, and the resulting solution was added to the above solution. The mixture was placed in the water bath shaker and shaken for reaction at 4° C. for 1 h, and then the reaction was terminated. The reaction solution was desalted and purified by a Sephadex G25 gel column (eluent: 20 mM aqueous histidine buffer solution at pH 5.5) to give a solution of the exemplary product ADC-6 (20 mM aqueous histidine buffer solution at pH 5.5; 18.94 mg, 4.96 mg/mL, 94.7% yield), which was then stored at 4° C.

LC-MS analysis was performed, and the DAR value was calculated to be n=4.48.

### Preparation Example ADC-7

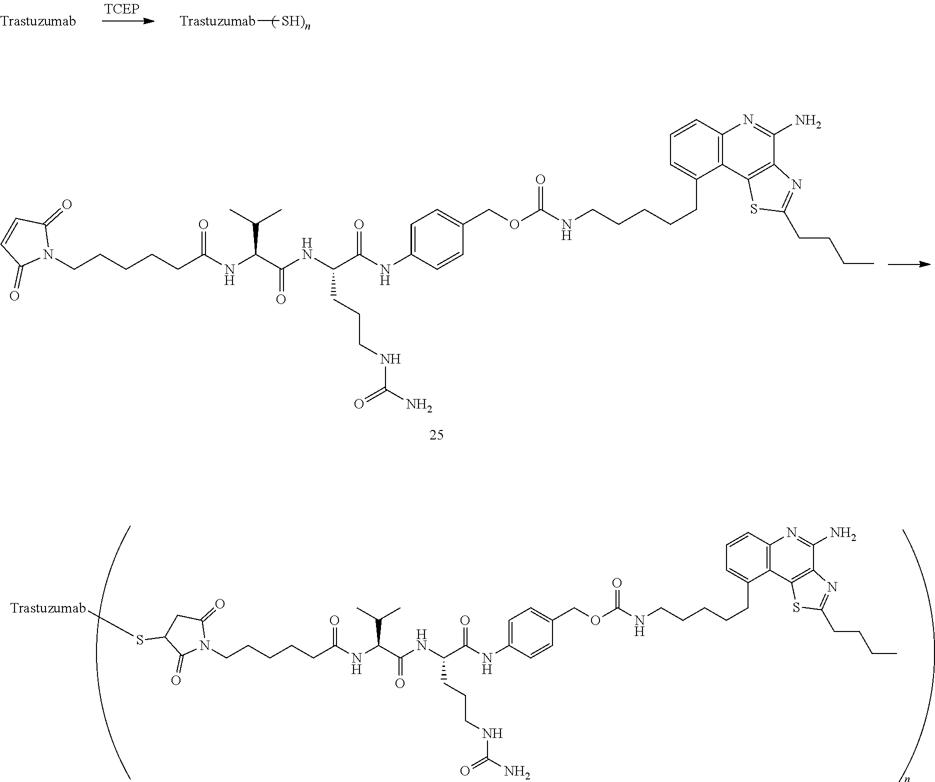

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 15 mg, 10 mg/mL, 0.103 μmol) was added a prepared aqueous solution of tris(2-carboxyethyl)phosphine (5 mM, 0.062 mL, 0.309 μmol) at 22° C. The mixture was placed in a water bath shaker and shaken for reaction at 22° C. for 3 h, and then the reaction was terminated.

25 (1.0 mg, 1.03 μmol) was dissolved in 0.10 mL of DMSO, and the resulting solution was added to the above solution. The mixture was placed in the water bath shaker and shaken for reaction at 22° C. for 1 h. A solution of 25 (1.0 mg, 1.03 μmol) in 0.10 mL of DMSO was added, the mixture was shaken for reaction at 22° C. for 1 h, and then the reaction was terminated. The reaction solution was desalted and purified by a Sephadex G25 gel column (eluent: 20 mM aqueous histidine buffer solution at pH 5.5) to give a solution of the exemplary product ADC-7 (20 mM aqueous histidine buffer solution at pH 5.5; 14 mg, 1.33 mg/mL, 93.3% yield), which was then stored at 4° C.

LC-MS analysis was performed, and the DAR value was calculated to be n=3.87.

### Preparation Example ADC-8

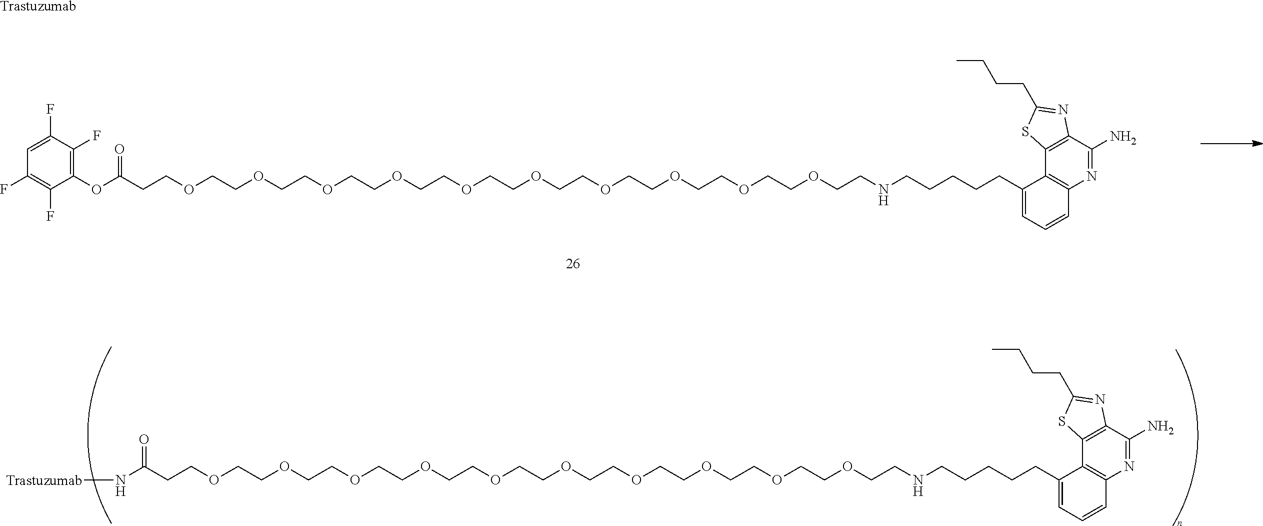

Compound 0735 (2.0 mg, 2.03 μmol) was dissolved in 0.20 mL of DMSO, and the resulting solution was added to an aqueous buffer solution of antibody trastuzumab (0.05 M aqueous PB buffer solution at pH 7.0; 25 mg, 5 mg/mL, 0.172 μmol). The mixture was placed in a water bath shaker and shaken for reaction at 37° C. for 2 h, and then the reaction was quenched with 200 mM succinic acid (pH 2.356, 1/8 V). The reaction solution was desalted and purified by a Sephadex G25 gel column (eluent: 20 mM aqueous succinate buffer solution at pH 5.0) to give an aqueous solution of the product ADC-8 (20 mM aqueous succinate buffer solution at pH 5.0; 20.27 mg, 5.08 mg/mL, 81.1% yield), which was then stored at 4° C.

LC-MS analysis was performed, and the DAR value was calculated to be n=3.73.

### Preparation Example ADC-9

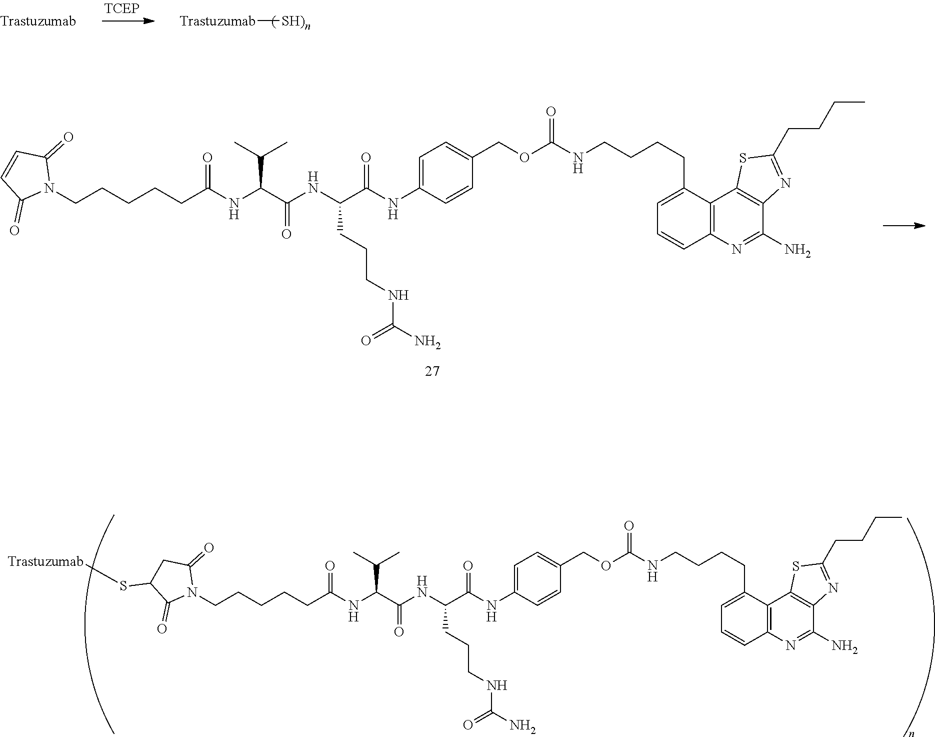

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 27 mg, 10 mg/mL, 0.186 μmol) was added a prepared aqueous solution of tris(2-carboxyethyl)phosphine (5 mM, 0.134 mL, 0.670 μmol) at 22° C. The mixture was placed in a water bath shaker and shaken for reaction at 22° C. for 3 h, and then the reaction was terminated.

27 (2.6 mg, 2.79 μmol) was dissolved in 0.26 mL of a mixed solution of DMSO and DMA (6:14), and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-9 (20 mM aqueous histidine buffer solution at pH 5.5; 12 mg, 3.05 mg/mL, 44.4% yield), which was then stored at 4° C.

LC-MS analysis was performed, and the DAR value was calculated to be n=3.69.

### Preparation Example ADC-10

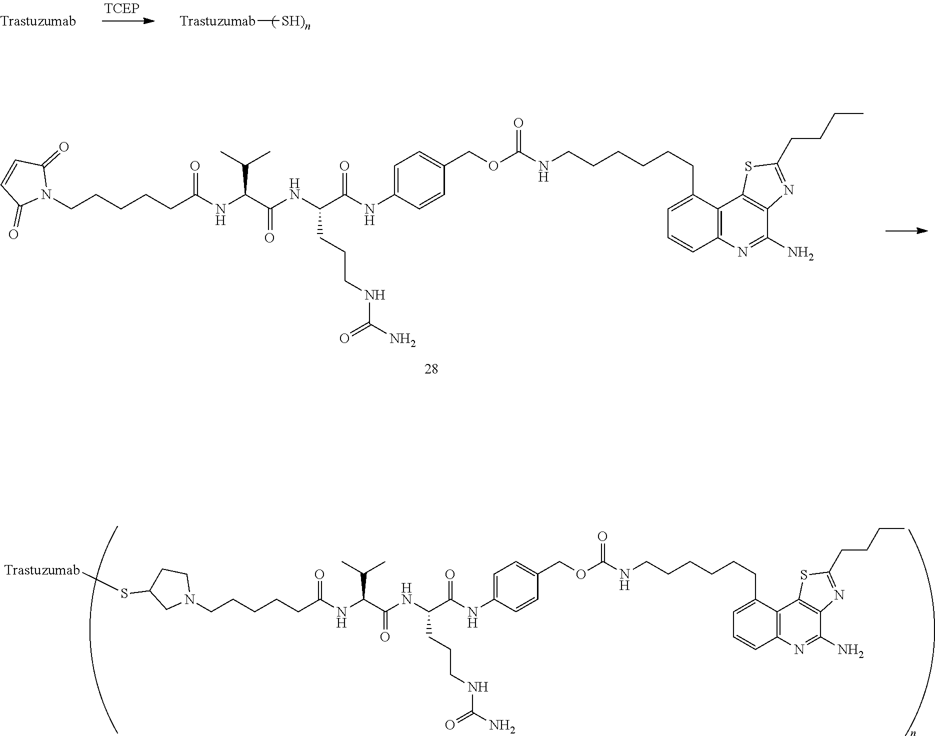

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 30 mg, 10 mg/mL, 0.206 μmol) was added a prepared aqueous solution of tris(2-carboxyethyl)phosphine (5 mM, 0.157 mL, 0.783 μmol) at 22° C. The mixture was placed in a water bath shaker and shaken for reaction at 22° C. for 3 h, and then the reaction was terminated.

28 (3.0 mg, 3.09 μmol) was dissolved in 0.30 mL of a mixed solution of DMSO and DMA (6:14), and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-10 (20 mM aqueous histidine buffer solution at pH 5.5; 20 mg, 3.96 mg/mL, 66.7% yield), which was then stored at 4° C.

LC-MS analysis was performed, and the DAR value was calculated to be n=3.54.

### Preparation Example ADC-12

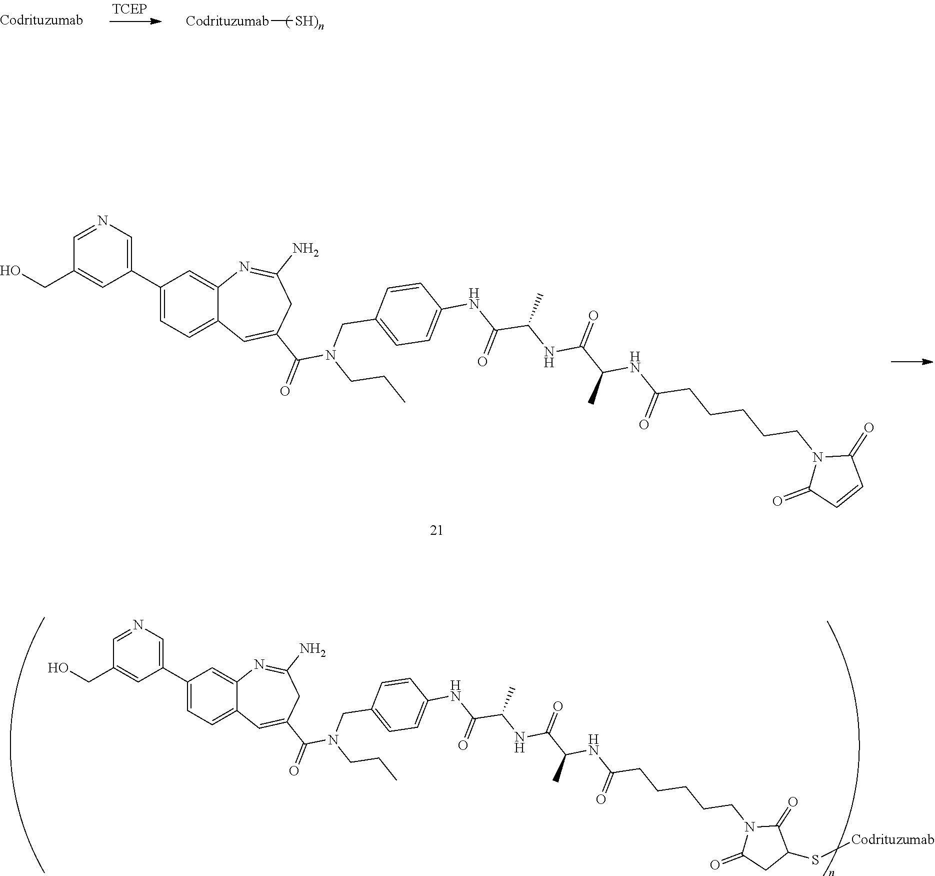

To an aqueous buffer solution of antibody codrituzumab (Gibco PBS buffer at pH 7.4; 15 mg, 6.85 mg/mL, 0.103 μmol) was added a prepared aqueous solution of tris(2-carboxyethyl)phosphine (5 mM, 0.058 mL, 0.288 μmol) at 22° C. The mixture was placed in a water bath shaker and shaken for reaction at 22° C. for 2 h, and then the reaction was terminated.

21 (1.2 mg, 1.54 μmol) was dissolved in 0.12 mL of DMSO, and the resulting solution was added to the above solution. The mixture was placed in the water bath shaker and shaken for reaction at 4° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-12 (20 mM aqueous histidine buffer solution at pH 5.5; 13 mg, 3.92 mg/mL, 86.7% yield), which was then stored at 4° C.

LC-MS analysis was performed, and the DAR value was calculated to be n=4.24.

### Preparation Example ADC-13

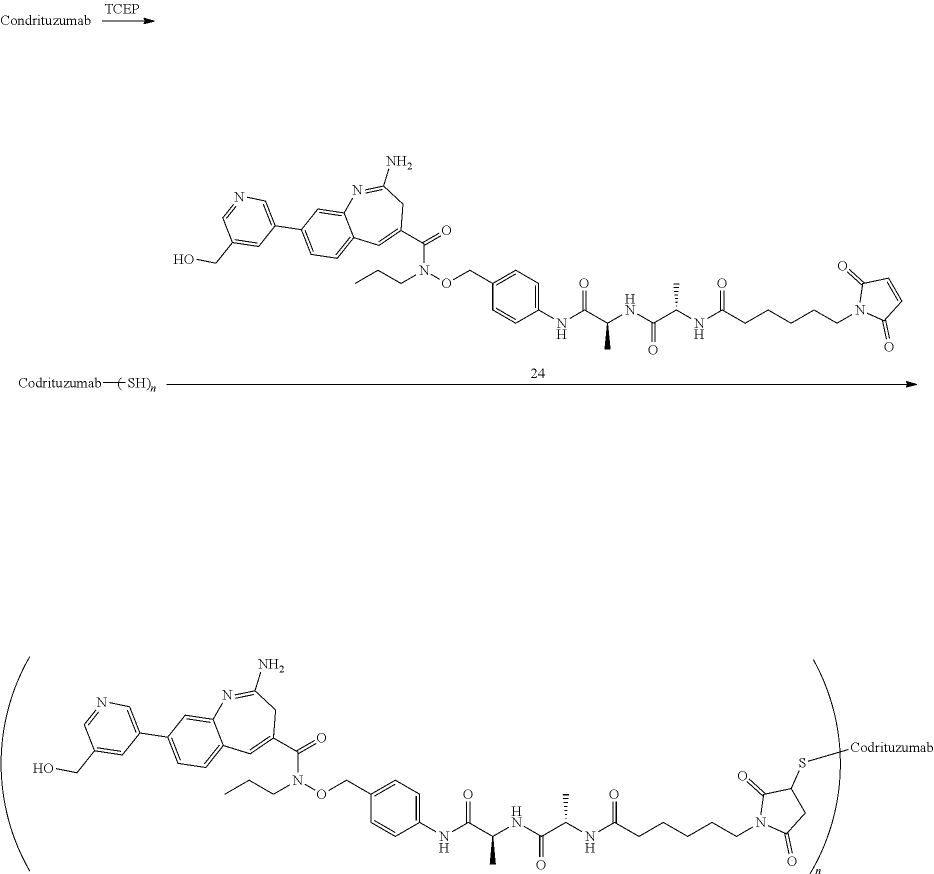

To an aqueous buffer solution of antibody codrituzumab (Gibco PBS buffer at pH 7.4; 25 mg, 6.85 mg/mL, 0.172 μmol) was added a prepared aqueous solution of tris(2-carboxyethyl)phosphine (5 mM, 0.096 mL, 0.482 μmol) at 22° C. The mixture was placed in a water bath shaker and shaken for reaction at 22° C. for 2 h, and then the reaction was terminated.

24 (2.1 mg, 2.58 μmol) was dissolved in 0.21 mL of DMSO, and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-13 (20 mM aqueous histidine buffer solution at pH 5.5; 20 mg, 4.80 mg/mL, 80.0% yield), which was then stored at 4° C.

LC-MS analysis was performed, and the DAR value was calculated to be n=3.76.

### Preparation Example ADC-14

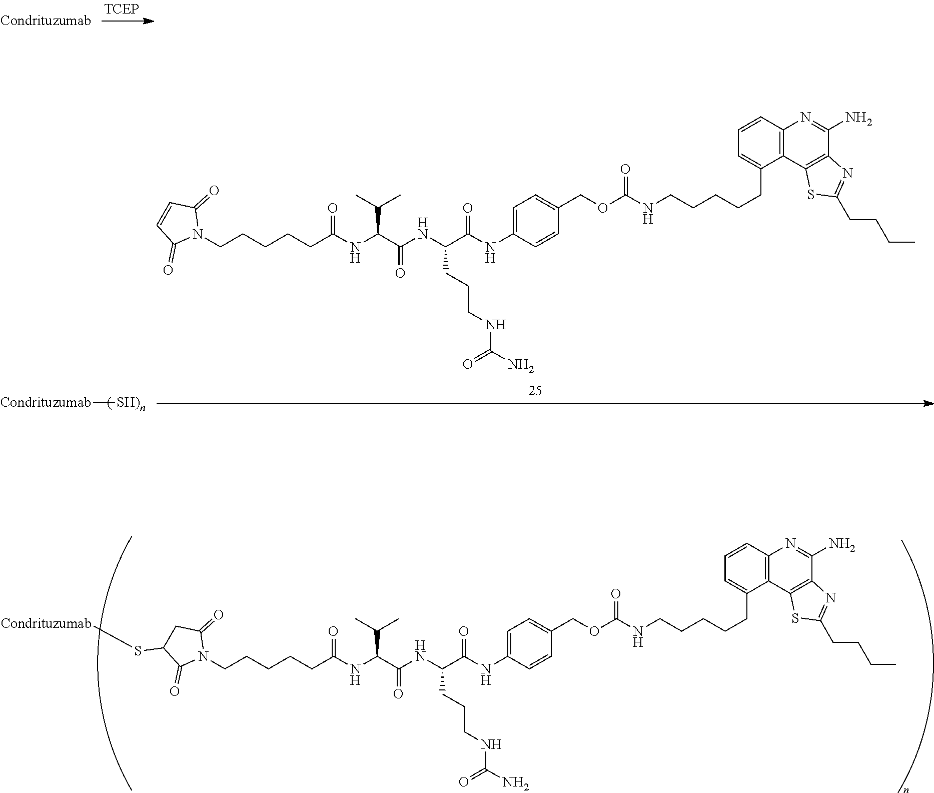

To an aqueous buffer solution of antibody codrituzumab (Gibco PBS buffer at pH 7.4; 21.09 mg, 6.84 mg/mL, 0.145 μmol) was added a prepared aqueous solution of tris(2-carboxyethyl)phosphine (5 mM, 0.087 mL, 0.435 μmol) at 22° C. The mixture was placed in a water bath shaker and shaken for reaction at 22° C. for 2 h, and then the reaction was terminated.

25 (2.0 mg, 2.18 μmol) was dissolved in 0.20 mL of a mixed solution of DMSO and DMA (3.9:16.1), and the resulting solution was added to the above solution. The mixture was placed in the water bath shaker and shaken for reaction at 4° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-14 (20 mM aqueous histidine buffer solution at pH 5.5; 15 mg, 2.34 mg/mL, 71.10% yield), which was then stored at 4° C.

LC-MS analysis was performed, and the DAR value was calculated to be n=4.22.

### Preparation Example ADC-15

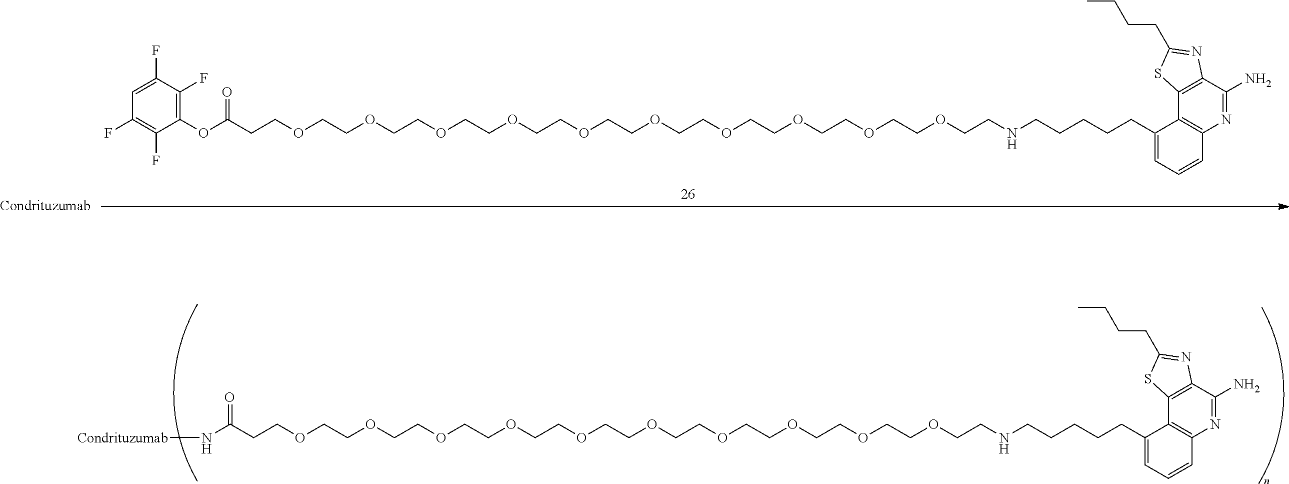

Compound 26 (1.6 mg, 1.64 μmol) was dissolved in 0.20 mL of DMSO, and the resulting solution was added to an aqueous buffer solution of antibody codrituzumab (50 mM PB buffer at pH 7.05, containing 2 mM EDTA; 17 mg, 4.22 mg/mL, 0.117 μmol). The mixture was placed in a water bath shaker and shaken for reaction at 22° C. for 2 h, and then the reaction was terminated. The reaction solution was desalted and purified by a Sephadex G25 gel column (eluent: 20 mM aqueous succinate buffer solution at pH 5.0) to give an aqueous solution of the product ADC-15 (20 mM aqueous succinate buffer solution at pH 5.0; 12.71 mg, 3.53 mg/mL, 74.8% yield), which was then stored at 4° C.

LC-MS analysis was performed, and the DAR value was calculated to be n=3.67.

### Preparation Example ADC-16

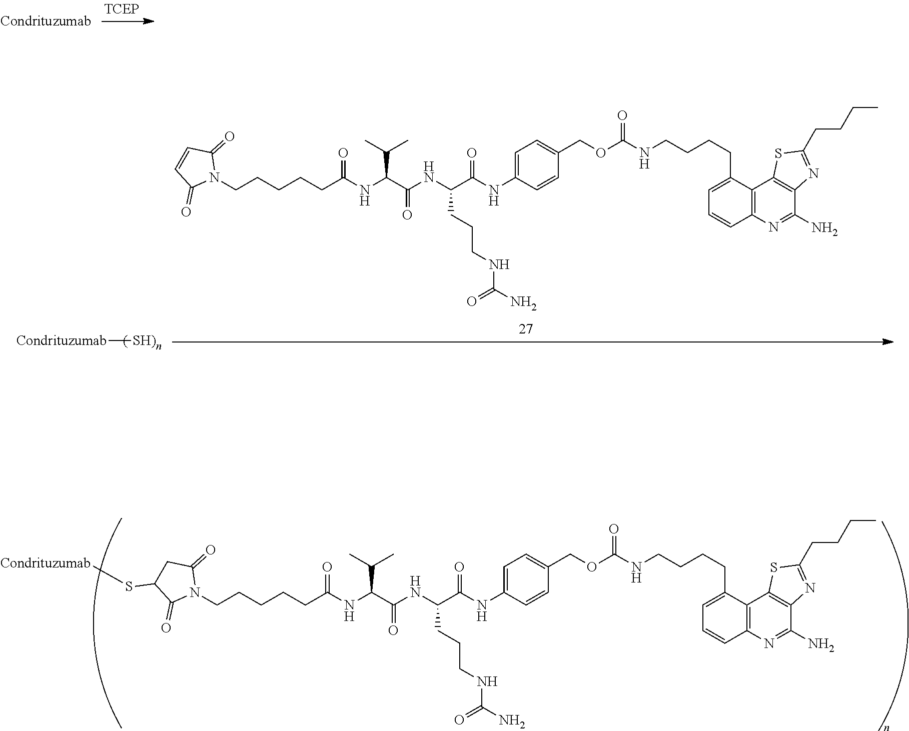

To an aqueous buffer solution of antibody codrituzumab (Gibco PBS buffer at pH 7.4; 25 mg, 8.34 mg/mL, 0.172 μmol) was added a prepared aqueous solution of tris(2-carboxyethyl)phosphine (5 mM, 0.096 mL, 0.482 μmol) at 22° C. The mixture was placed in a water bath shaker and shaken for reaction at 22° C. for 2 h, and then the reaction was terminated.

27 (2.4 mg, 2.58 μmol) was dissolved in 0.24 mL of a mixed solution of DMSO and DMA (4.7:15.3), and the resulting solution was added to the above solution. The mixture was placed in the water bath shaker and shaken for reaction at 4° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-16 (20 mM aqueous histidine buffer solution at pH 5.5; 18.69 mg, 3.60 mg/mL, 74.8% yield), which was then stored at 4° C.

LC-MS analysis was performed, and the DAR value was calculated to be n=4.13.

### Preparation Example ADC-17

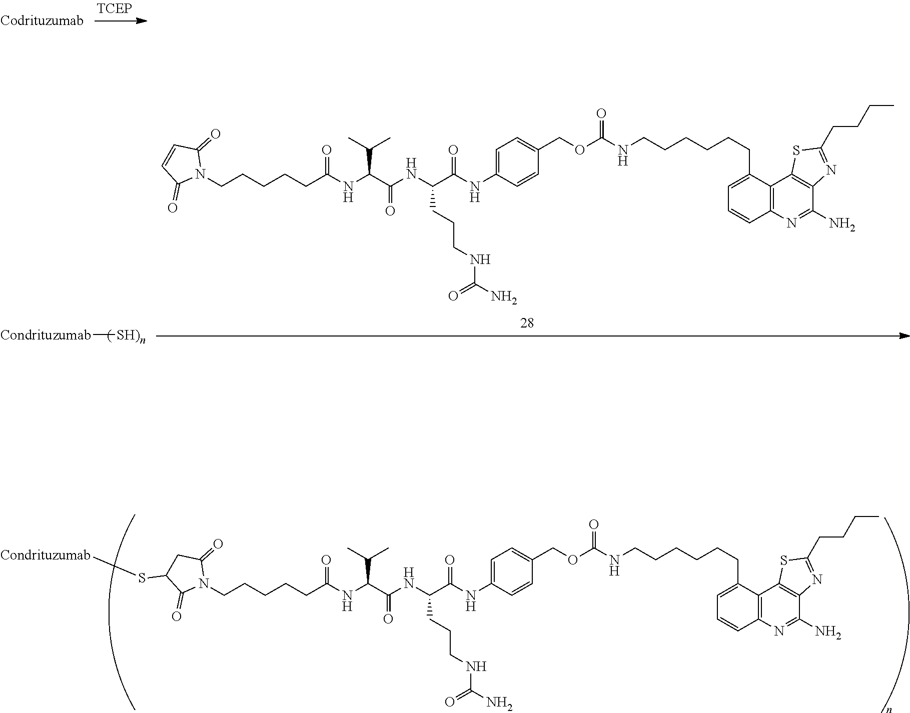

To an aqueous buffer solution of antibody codrituzumab (Gibco PBS buffer at pH 7.4; 18.7 mg, 6.84 mg/mL, 0.128 μmol) was added a prepared aqueous solution of tris(2-carboxyethyl)phosphine (5 mM, 0.077 mL, 0.384 μmol) at 22° C. The mixture was placed in a water bath shaker and shaken for reaction at 22° C. for 2 h, and then the reaction was terminated.

28 (1.8 mg, 1.92 μmol) was dissolved in 0.18 mL of a mixed solution of DMSO and DMA (4.2:15.8), and the resulting solution was added to the above solution. The mixture was placed in the water bath shaker and shaken for reaction at 4° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-17 (20 mM aqueous histidine buffer solution at pH 5.5; 13.69 mg, 3.47 mg/mL, 73.2% yield), which was then stored at 4° C.

LC-MS analysis was performed, and the DAR value was calculated to be n=4.15.

### Preparation Example ADC-18

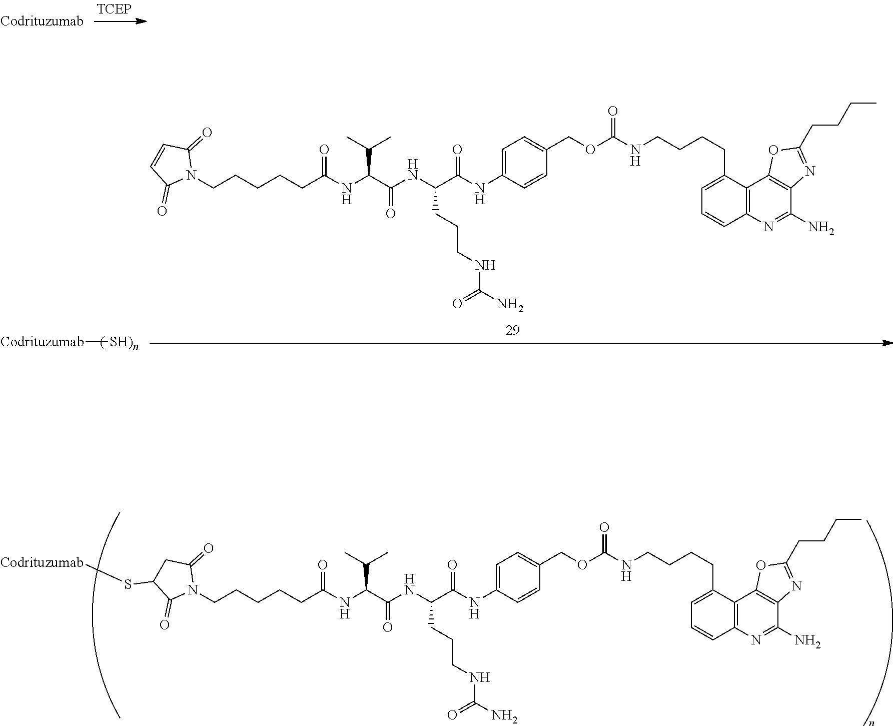

To an aqueous buffer solution of antibody codrituzumab (Gibco PBS buffer at pH 7.4; 25 mg, 8.34 mg/mL, 0.172 μmol) was added a prepared aqueous solution of tris(2-carboxyethyl)phosphine (5 mM, 0.089 mL, 0.447 μmol) at 22° C. The mixture was placed in a water bath shaker and shaken for reaction at 22° C. for 2 h, and then the reaction was terminated.

Compound 29 (9.4 mg, 10.32 μmol) was dissolved in 0.94 mL of DMSO, and the resulting solution was added to the above solution. The mixture was placed in the water bath shaker and shaken for reaction at 22° C. for 2 h, and then the reaction was terminated. The reaction solution was desalted and purified by a Sephadex G25 gel column (eluent: 20 mM aqueous histidine buffer solution at pH 5.5) to give a solution of the exemplary product ADC-18 (20 mM aqueous histidine buffer solution at pH 5.5; 19.0 mg, 3.32 mg/mL, 76% yield), which was then stored at 4° C.

LC-MS analysis was performed, and the DAR value was calculated to be n=3.47.

### Preparation Example ADC-19

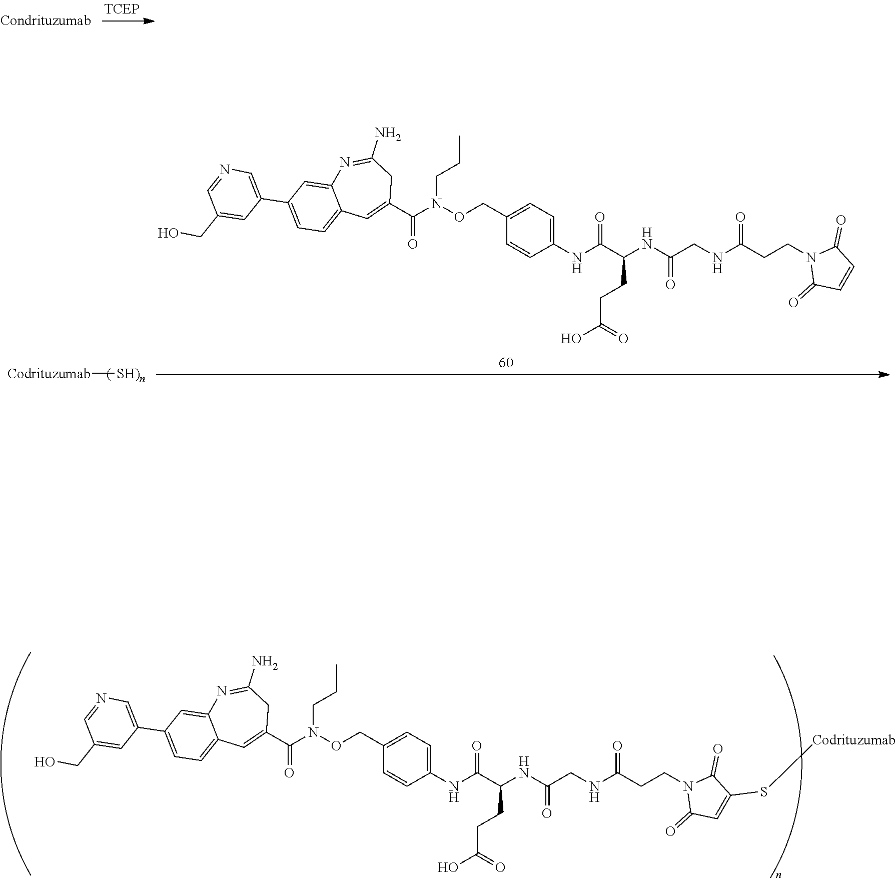

To an aqueous buffer solution of antibody codrituzumab (Gibco PBS buffer at pH 7.4; 15 mg, 8.00 mg/mL, 0.101 μmol) was added a prepared aqueous solution of tris(2-carboxyethyl)phosphine (5 mM, 0.067 mL, 0.333 μmol) at 22° C. The mixture was placed in a water bath shaker and shaken for reaction at 22° C. for 2 h, and then the reaction was terminated.

Compound 60 (2.6 mg, 3.23 μmol) was dissolved in 0.32 mL of DMSO, and the resulting solution was added to the above solution. The mixture was placed in the water bath shaker and shaken for reaction at 22° C. for 3 h, and then the reaction was terminated. The reaction solution was purified by a HiTrap desalting column (5 mL×3, eluent: 20 mM aqueous histidine buffer solution at pH 5.5) to give a solution of the exemplary product ADC-19 (20 mM aqueous histidine buffer solution at pH 5.5; 10.5 mg, 1.81 mg/mL, 70.0% yield), which was then stored at 4° C.

LC-MS analysis was performed, and the DAR value was calculated to be n=4.28.

### Preparation Example ADC-20

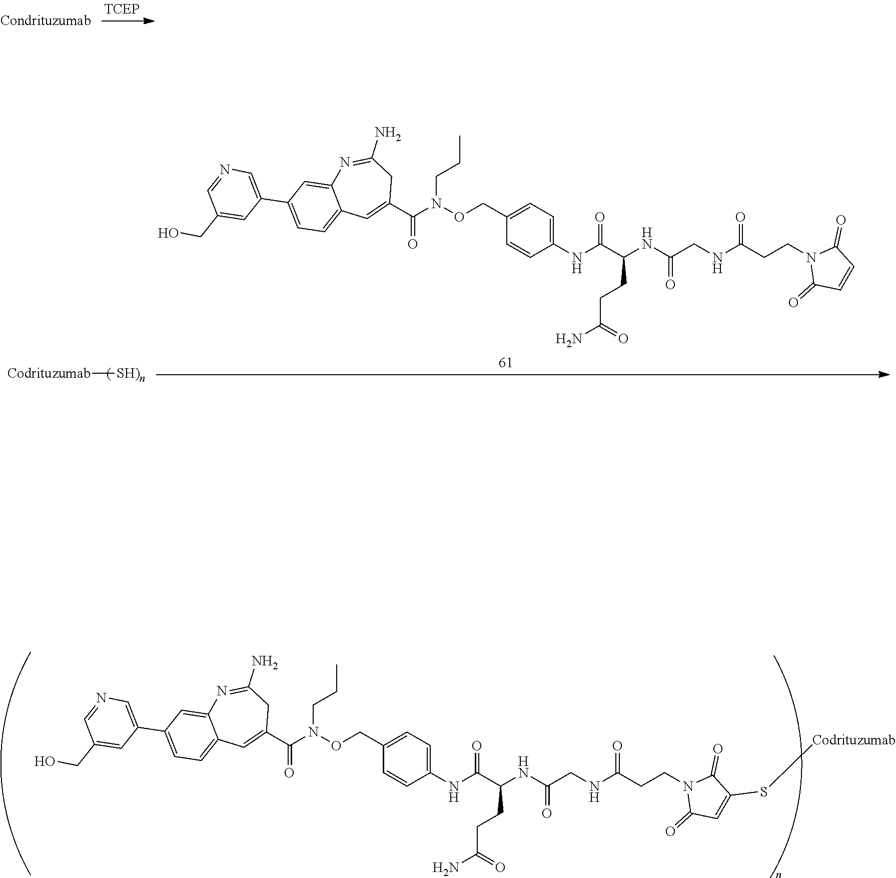

To an aqueous buffer solution of antibody codrituzumab (Gibco PBS buffer at pH 7.4; 19.1 mg, 8.00 mg/mL, 0.129 μmol) was added a prepared aqueous solution of tris(2-carboxyethyl)phosphine (5 mM, 0.075 mL, 0.374 μmol) at 22° C. The mixture was placed in a water bath shaker and shaken for reaction at 22° C. for 2 h, and then the reaction was terminated.

Compound 61 (1.6 mg, 1.94 μmol) was dissolved in 0.19 mL of DMSO, and the resulting solution was added to the above solution. The mixture was placed in the water bath shaker and shaken for reaction at 22° C. for 3 h, and then the reaction was terminated. The reaction solution was purified by a HiPrep desalting column (53 mL, eluent: 20 mM aqueous histidine buffer solution at pH 5.5) to give a solution of the exemplary product ADC-20 (20 mM aqueous histidine buffer solution at pH 5.5; 16.9 mg, 4.43 mg/mL, 88.5% yield), which was then stored at 4° C.

LC-MS analysis was performed, and the DAR value was calculated to be n=4.09.

### Preparation Example ADC-21

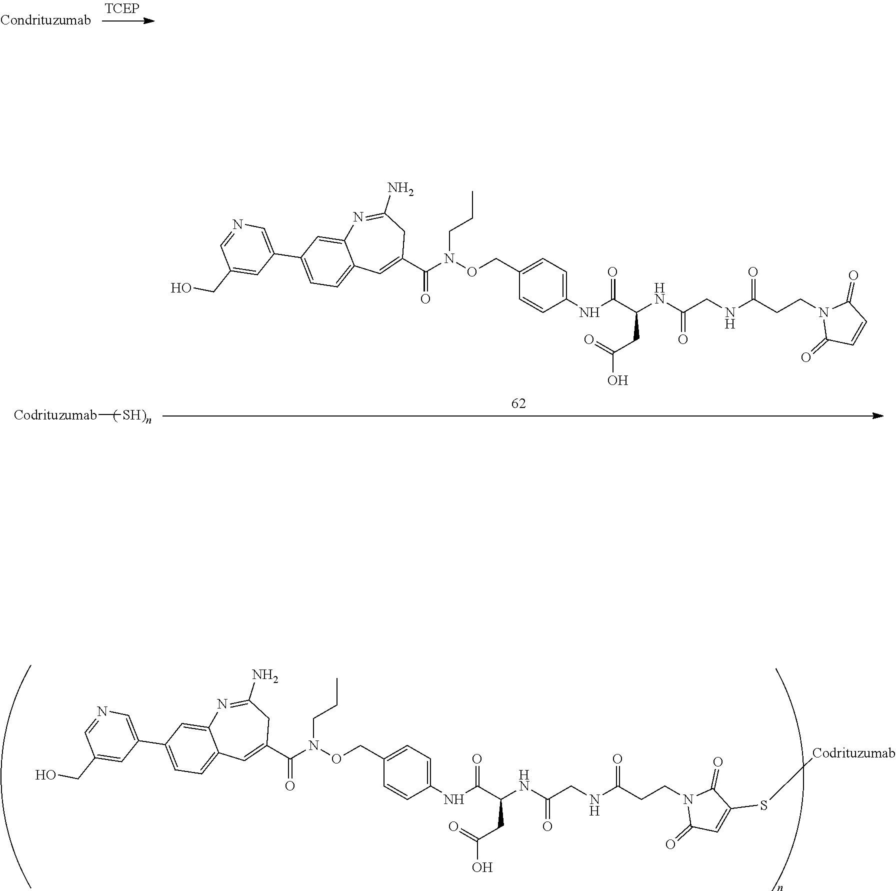

To an aqueous buffer solution of antibody codrituzumab (Gibco PBS buffer at pH 7.4; 12 mg, 11.5 mg/mL, 0.081 μmol) was added a prepared aqueous solution of tris(2-carboxyethyl)phosphine (5 mM, 0.042 mL, 0.211 μmol) at 22° C. The mixture was placed in a water bath shaker and shaken for reaction at 22° C. for 3 h, and then the reaction was terminated.

Compound 62 (0.45 mg, 0.567 μmol) was dissolved in 0.057 mL of DMSO, and the resulting solution was added to the above solution. The mixture was placed in the water bath shaker and shaken for reaction at 4° C. for 2 h, and then the reaction was terminated. The reaction solution was purified by a HiTrap desalting column (5 mL×2, eluent: 20 mM aqueous histidine buffer solution at pH 5.5) to give a solution of the exemplary product ADC-21 (20 mM aqueous histidine buffer solution at pH 5.5; 6.5 mg, 3.78 mg/mL, 54.2% yield), which was then stored at 4° C.

LC-MS analysis was performed, and the DAR value was calculated to be n=4.05.

### Preparation Example ADC-22

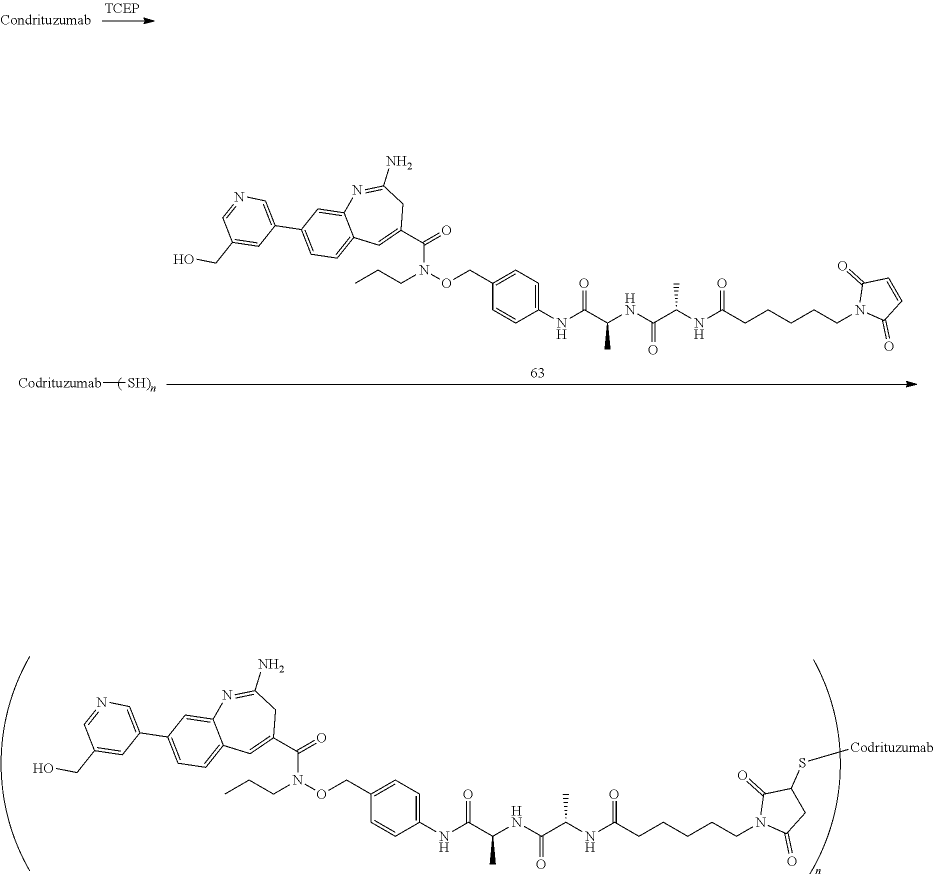

To an aqueous buffer solution of antibody codrituzumab (Gibco PBS buffer at pH 7.4; 14 mg, 8.0 mg/mL, 0.094 μmol) was added a prepared aqueous solution of tris(2-carboxyethyl)phosphine (5 mM, 0.053 mL, 0.263 μmol) at 22° C. The mixture was placed in a water bath shaker and shaken for reaction at 22° C. for 2 h, and then the reaction was terminated.

Compound 63 (1.16 mg, 1.41 μmol) was dissolved in 0.14 mL of DMSO, and the resulting solution was added to the above solution. The mixture was placed in the water bath shaker and shaken for reaction at 4° C. for 2.5 h, and then the reaction was terminated. The reaction solution was purified by a HiTrap desalting column (5 mL×3, eluent: 20 mM aqueous histidine buffer solution at pH 5.5) to give a solution of the exemplary product ADC-22 (20 mM aqueous histidine buffer solution at pH 5.5; 7.95 mg, 3.95 mg/mL, 56.8% yield), which was then stored at 4° C.

LC-MS analysis was performed, and the DAR value was calculated to be n=3.70.

### Preparation Example ADC-23

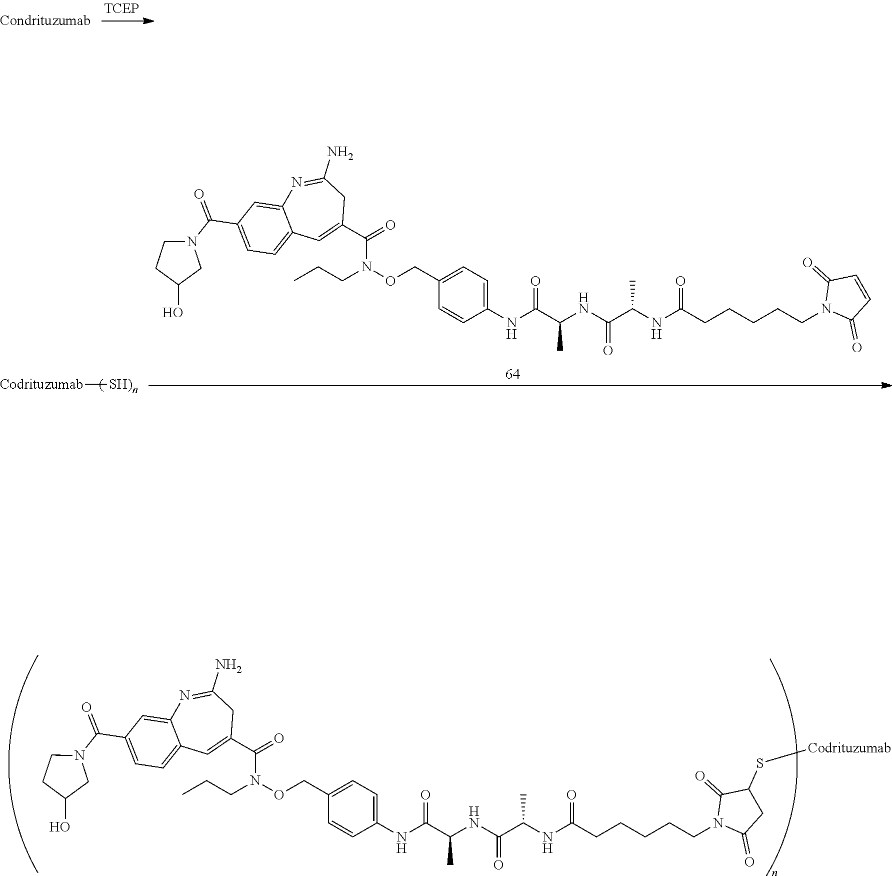

To an aqueous buffer solution of antibody codrituzumab (Gibco PBS buffer at pH 7.4; 30.5 mg, 11.0 mg/mL, 0.206 μmol) was added a prepared aqueous solution of tris(2-carboxyethyl)phosphine (5 mM, 0.062 mL, 0.309 μmol) at 22° C. The mixture was placed in a water bath shaker and shaken for reaction at 22° C. for 3 h, and then the reaction was terminated.

Compound 64 (2.5 mg, 3.09 μmol) was dissolved in 0.31 mL of DMSO, and the resulting solution was added to the above solution. The mixture was placed in the water bath shaker and shaken for reaction at 22° C. for 2 h, and then the reaction was terminated. The reaction solution was purified by a desalting column (53 mL, eluent: 20 mM aqueous histidine buffer solution at pH 5.5) to give a solution of the exemplary product ADC-23 (20 mM aqueous histidine buffer solution at pH 5.5; 22.03 mg, 6.77 mg/mL, 72.2% yield), which was then stored at 4° C.

LC-MS analysis was performed, and the DAR value was calculated to be n=2.00.

### Preparation Example ADC-24

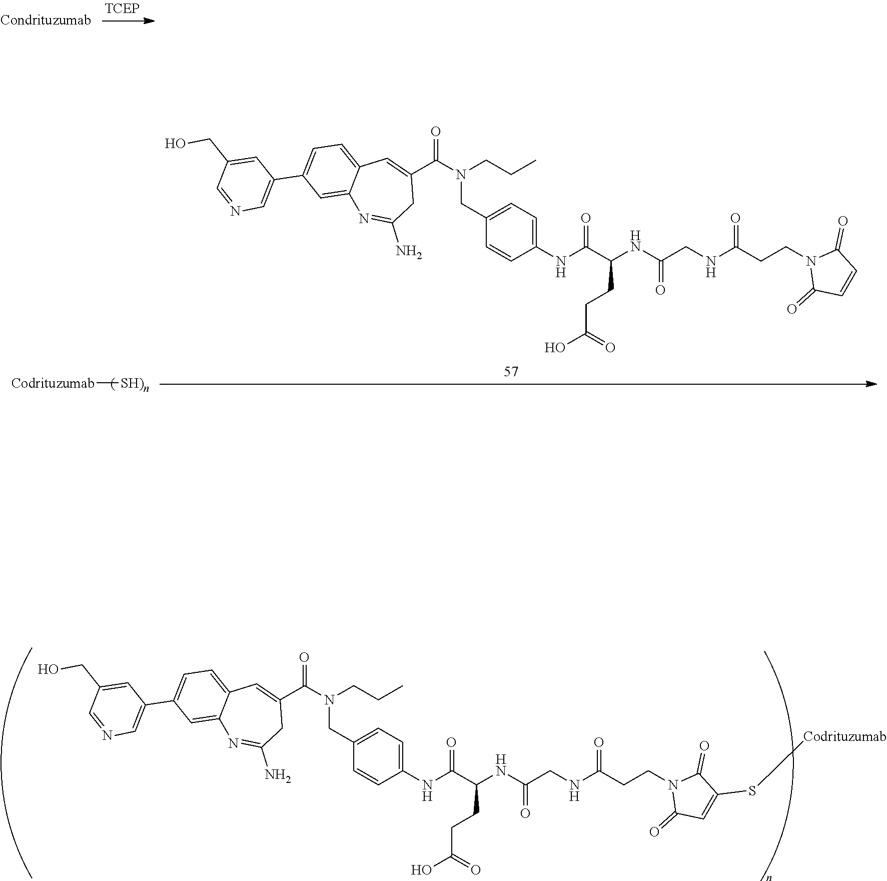

To an aqueous buffer solution of antibody codrituzumab (Gibco PBS buffer at pH 7.4; 25 mg, 8.34 mg/mL, 0.169 μmol) was added a prepared aqueous solution of tris(2-carboxyethyl)phosphine (5 mM, 0.095 mL, 0.473 μmol) at 22° C. The mixture was placed in a water bath shaker and shaken for reaction at 22° C. for 2 h, and then the reaction was terminated.

Compound 57 (2.0 mg, 2.54 μmol) was dissolved in 0.20 mL of DMSO, and the resulting solution was added to the above solution. The mixture was placed in the water bath shaker and shaken for reaction at 4° C. for 2.5 h, and then the reaction was terminated. The reaction solution was purified by a HiTrap desalting column (5 mL×2, eluent: 20 mM aqueous histidine buffer solution at pH 5.5) to give a solution of the exemplary product ADC-24 (20 mM aqueous histidine buffer solution at pH 5.5; 19 mg, 5.30 mg/mL, 76.0% yield), which was then stored at 4° C.

LC-MS analysis was performed, and the DAR value was calculated to be n=4.03.

### Preparation Example ADC-25 (Control ADC1)

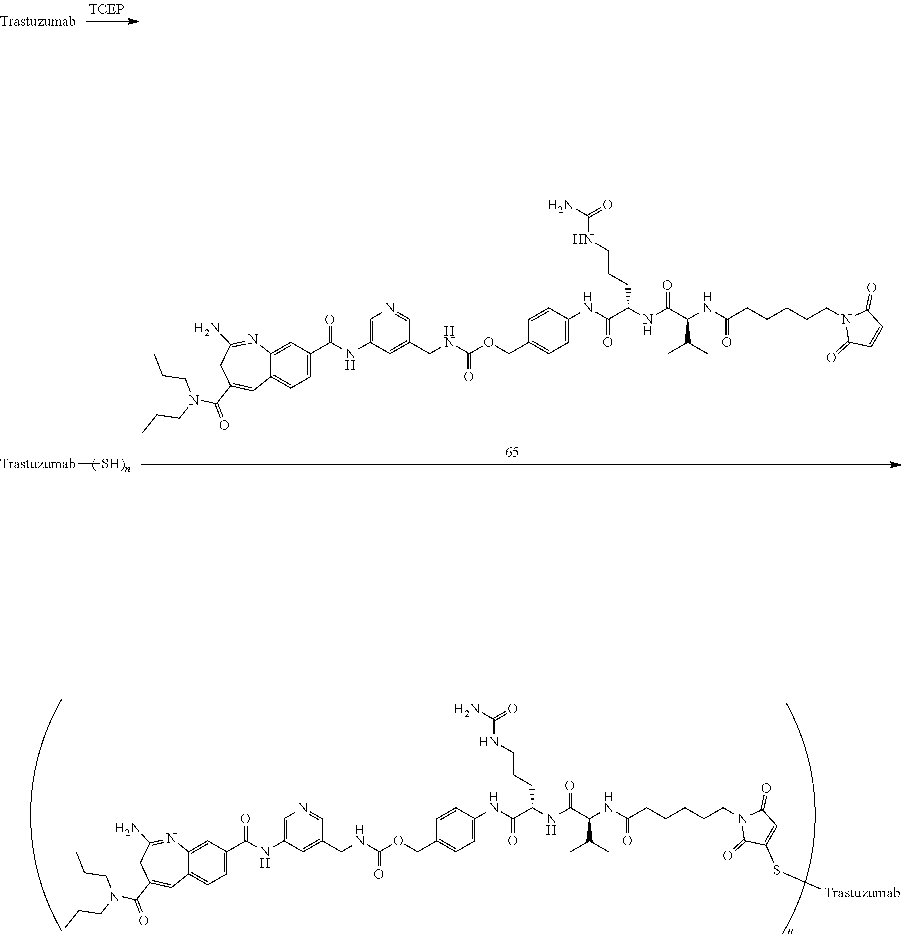

To an aqueous buffer solution of antibody trastuzumab (buffer consisting of 1.65 g/L succinic acid, 1.02 g/L NaOH and 6.30 g/L NaCl at pH 6.0, containing 2 mM EDTA; 40 mg, 10 mg/mL, 0.270 μmol) was added a prepared aqueous solution of tris(2-carboxyethyl)phosphine (5 mM, 0.432 mL, 2.160 μmol) at 22° C. The mixture was placed in a water bath shaker and shaken for reaction at 22° C. for 3 h, and then the reaction was terminated.

Compound 65 (4.9 mg, 4.752 μmol) was dissolved in 0.49 mL of DMSO, and the resulting solution was added to the above solution. The mixture was placed in the water bath shaker and shaken for reaction at 22° C. for 2 h, and then the reaction was terminated. The reaction solution was purified by a Spin desalting column (10 mL, eluent: 20 mM aqueous histidine buffer solution at pH 5.5) to give a solution of the exemplary product ADC-25 (20 mM aqueous histidine buffer solution at pH 5.5; 33 mg, 7.54 mg/mL, 82.5% yield), which was then stored at 4° C.

LC-MS analysis was performed, and the DAR value was calculated to be n=6.35.

### Preparation Example ADC-26 (Control ADC2)

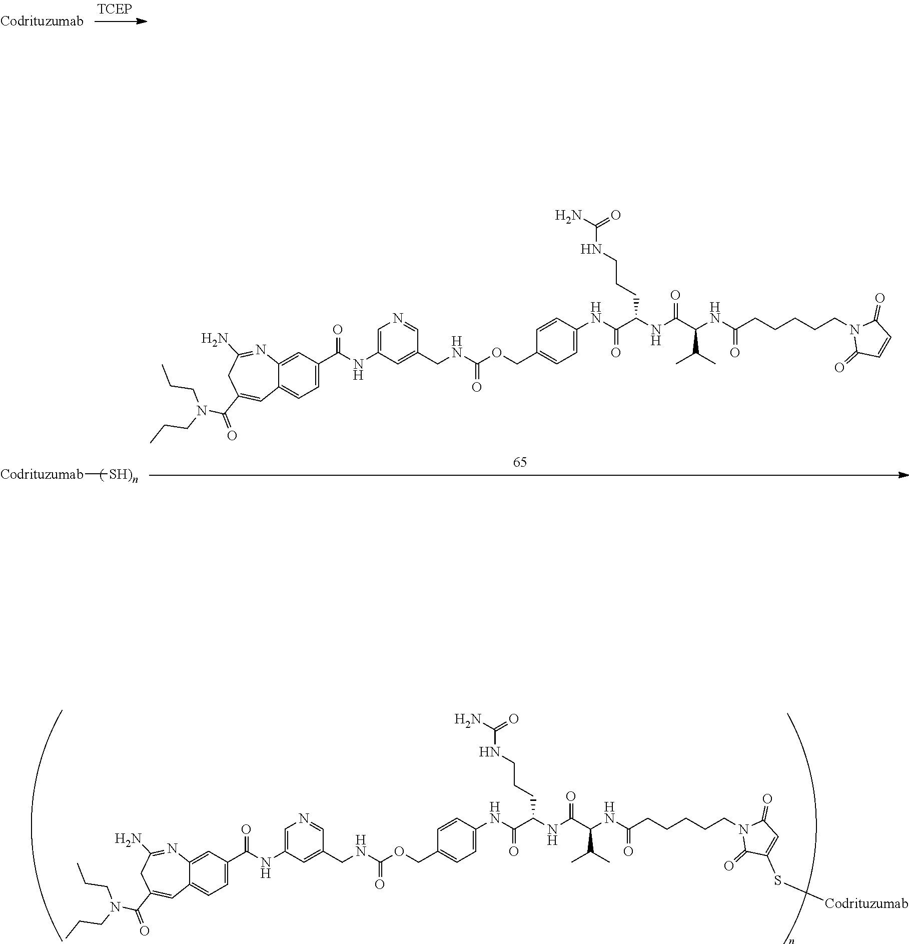

To an aqueous buffer solution of antibody codrituzumab (Gibco PBS buffer at pH 7.4; 21.09 mg, 6.85 mg/mL, 0.142 μmol) was added a prepared aqueous solution of tris(2-carboxyethyl)phosphine (5 mM, 0.080 mL, 0.398 μmol) at 22° C. The mixture was placed in a water bath shaker and shaken for reaction at 22° C. for 2 h, and then the reaction was terminated.

Compound 65 (2.2 mg, 2.13 μmol) was dissolved in 0.22 mL of DMSO, and the resulting solution was added to the above solution. The mixture was placed in the water bath shaker and shaken for reaction at 4° C. for 2 h, and then the reaction was terminated. The reaction solution was purified by a Spin desalting column (10 mL, eluent: 20 mM aqueous histidine buffer solution at pH 5.5) to give a solution of the exemplary product ADC-26 (20 mM aqueous histidine buffer solution at pH 5.5; 15.3 mg, 2.77 mg/mL, 72.5% yield), which was then stored at 4° C.

LC-MS analysis was performed, and the DAR value was calculated to be n=4.09.

### Preparation Example ADC-27 (Control ADC3)

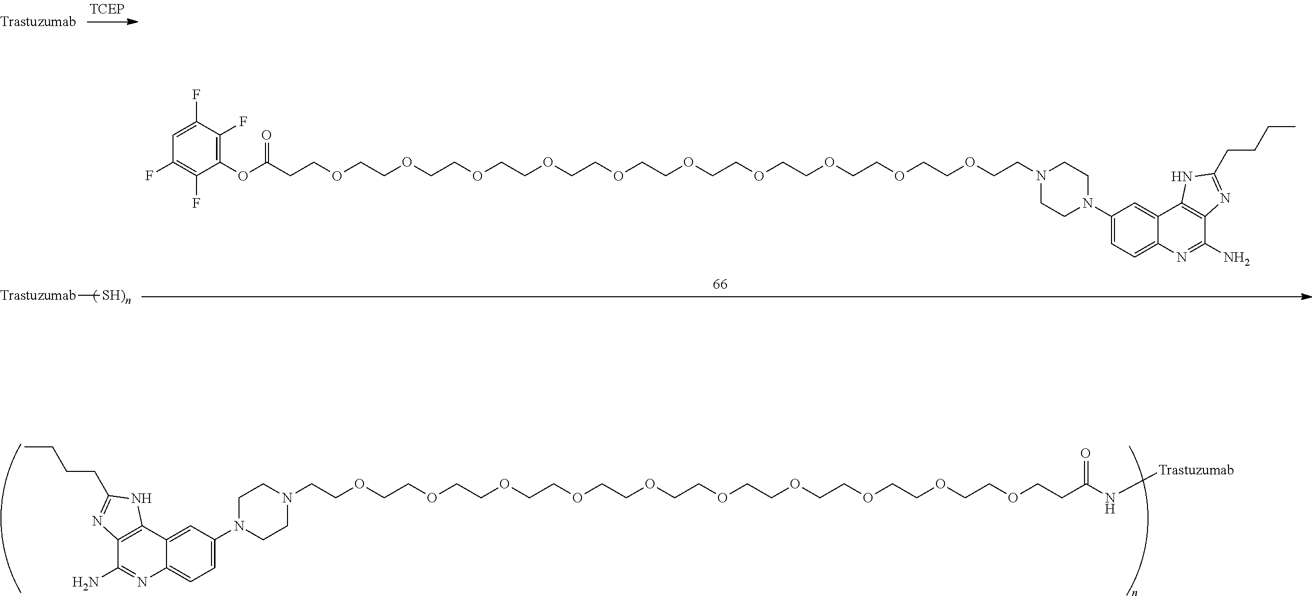

Compound 66 (2.7 mg, 2.74 μmol) was dissolved in 0.27 mL of DMA, and the resulting solution was added to an aqueous buffer solution of antibody trastuzumab (50 mM PB buffer at pH 7.0; 30 mg, 5 mg/mL, 0.203 μmol). The mixture was placed in a water bath shaker and shaken for reaction at 37° C. for 2 h, and then the reaction was terminated. The reaction was quenched by adding 1/8 volume of aqueous succinic acid solution (200 mM, pH=2.356), and the reaction solution was purified by a Spin desalting column (eluent: 20 mM aqueous succinate buffer solution at pH 5.0) to give an aqueous solution of the product ADC-27 (20 mM aqueous succinate buffer solution at pH 5.0; 23 mg, 4.08 mg/mL, 76.7% yield), which was then stored at 4° C.

LC-MS analysis was performed, and the DAR value was calculated to be n=3.45.

### Example 3

Agonistic Activity of Compounds on hTLR7 (Human Toll-Like Receptor 7) and hTLR8 (Human Toll-Like Receptor 8)

In this example, the agonistic activity of the compounds of the present application on hTLR7 (human Toll-like receptor 7) and hTLR8 (human Toll-like receptor 8) was assessed; the induction activity of the compounds on human PBMCs for TNF-α and IFN-α production was assessed in human PBMCs (human peripheral blood mononuclear cells).

All compounds were formulated in DMSO into 30 mM concentrated stock solutions and preserved at 4° C. The reference compound R₈₄₈ was formulated in DMSO into a 5 mg/mL concentrated stock solution and preserved in a freezer at −20° C. HEK-Blue hTLR7 and HEK-Blue hTLR8 cell lines were purchased from InvivoGen, and human PBMCs were purchased from Shanghai Saily Biotechnology Co., Ltd. (Cat. No. XFB—HP100B).

The reference compounds R848 (CAS No.: 144875-48-9) and VTX-2337 (CAS No.: 926927-61-9) were both purchased from Haoyuan Chemexpress, and their structures are shown below:

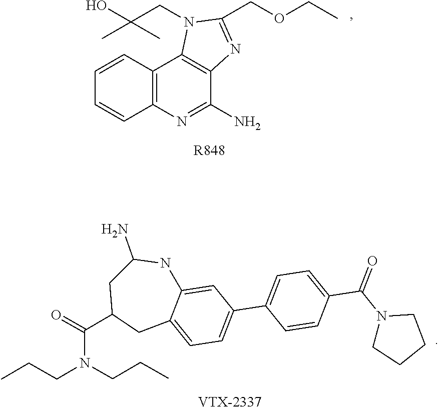

Other commonly used reagents are commercially available, for example, from the following sources:

| Abbreviation | Reagent | Supplier | Cat. No. |
|---|---|---|---|
| QUANTI-Blue ™ | QUANTI-Blue ™ | InvivoGen | rep-qbs |
| | detection agent | | |
| CellTiter-Glo | Fluorescent cell viability | Promega | G7573 |
| | detection kit | | |
| RPMI 1640 | RPMI 1640 medium | Invitrogen | 22400-089 |
| DMEM | DMEM medium | Invitrogen | 11960-051 |
| HI FBS | Thermally inactivated | Gibco | 10100-147C |
| | serum | | |
| L-Glutamine | L-Glutaminase | Gibco | 25030-081 |
| PS | Antibiotics (penicillin | Hyclone | SV30010 |
| | and streptomycin) | | |
| Blasticidin | Blasticidin | Invivogen | ant-bl-1 |
| Normocin | Neomycin | Invivogen | ant-nr-1 |
| Zeocin | Bleomycin | Invivogen | ant-zn-05 |
| TrypLE Express | Trypsin | Invitrogen | 12605-010 |
| DPBS | Dulbecco's phosphate- | Corning | 21-031-CVC |
| | buffered saline | | |
| Human TNF-α | Human TNF-α ELISA kit | DAKEWE | 1117202 |
| ELISA kit | | | |
| Human IFN-α | Human IFN-α ELISA kit | DAKEWE | 1110012 |
| ELISA kit | | | |

The major instruments used in this study were multimode microplate reader Flexstation III (Molecular Device), Envision (Perkin Elmer), M2e (Molecular Device), and Echo555 (Labcyte).

Agonistic Activity of Compounds on hTLR7 and hTLR8

Compound dilution: The compounds of the present application were diluted with DMSO, for example, to about 15 mM. The reference compounds were diluted and then sequentially added to cell plates. The compounds were serially 3-fold diluted to give a total of 10 concentrations and added to 96-well cell plates, with 2 replicate wells set for each concentration. DMSO was added to the negative control wells at 0.2 μL/well, and the reference compounds were added to the positive control wells. The final concentration of DMSO was 0.2%.

Cell plating: When an 80% confluence of HEK Blue hTLR7&8 cells in the culture flask was observed under a microscope, the culture medium was discarded, 10 mL of detection medium was added, and the cells were pipetted to form a single-cell suspension. The cells were counted using a cell counter and diluted to 500,000 cells/mL. The cells were plated at 100 μL/50,000 cells/well in the 96-well plates containing the compounds. The compounds and cells were co-incubated in an incubator at 37° C. with 5% CO₂ for 24 h.

Compound activity detection: 20 μL of cell supernatant was taken from each well and added to an experimental plate containing QUANTI-Blue™ reagent at 180 μL/well. The plate was incubated at 37° C. for 1 h, and then the absorbance at 650 nm (OD₆₅₀) was measured on the multimode microplate reader Flexstation III.

Cell viability detection: The chemiluminescent signal (RLU) was detected on the multimode microplate reader Flexstation III according to the instructions of Celltiter-Glo.

Compound activity data analysis: The OD₆₅₀ values were analyzed by GraphPad Prism software and fitted to give dose-response curves of the compounds. The EC₅₀ values of the compounds were calculated.

The details are shown in Table 1.

The results show that the compounds of the present application had significant activation activity on both TLR7 and TLR8, and the activation activity was superior to that of the controls VTX-2337 and R848.

### Induction Activity of Compounds on TNF-α and IFN-α in Human PBMCs

PBMC cells were treated with the compounds. The compounds of the present application were diluted with DMSO, for example, to about 15 mM. The reference compounds were diluted to 5 mg/mL and then sequentially added to cell plates. The compounds were serially 3-fold diluted to give a total of 8 concentrations and added to cell plates, with two replicate wells set for each concentration. DMSO was added to the negative control wells at 0.4 μL/well, and the reference compounds at 5 mg/mL were added to the positive control wells at 0.4 μL/well. The final concentration of DMSO was 0.2%. Frozen human PBMCs in cryopreservation tubes were quickly thawed in a 37° C. water bath and transferred into 50-mL centrifuge tubes. Pre-warmed RPMI1640 medium was gradually added, and the mixture was well mixed and centrifuged for 15 min before the supernatant was discarded. The cells were resuspended in a culture medium and then counted. The cell suspension was adjusted to 2.5×10⁶ cells/mL with the culture medium. Diluted cells were added at 200 μL/500,000 cells/well in the 96-well plates containing the compounds. The cell plates were incubated at 37° C. with 5% CO₂ for 24 h.

### TNF-α Content Assay:

Sample loading: 100 μL of serially diluted cytokine standard was added to each standard well, 100 μL of 10-fold diluted test sample was added to each sample well, and 100 μL of 1× dilution buffer R was added to each blank control well. Addition of detection antibody: 50 μL of biotinylated antibody working solution was added to each well. After mixing well, the plate was covered with a plate-sealing film, incubated at room temperature for 3 h, and washed 3 times, and 100 μL of streptavidin-HRP working solution was added to each well. The plate was covered with a plate-sealing film and incubated at room temperature for 20 min. The plate was then washed 3 times. Color development: 100 μL of TMB liquid was added to each well, and the plate was incubated at room temperature (18-25° C.) in the dark for 25 min. Termination of reaction: The reaction was terminated by quickly adding a stop solution at 100 μL/well. Plate reading: The reading at wavelength 450 nm was detected using an M2e instrument within 10 min after the termination.

### IFN-α Content Assay:

Sample loading: 100 μL of serially diluted cytokine standard was added to each standard well, 100 μL of 2-fold diluted test sample was added to each sample well, and 100 μL of 1× dilution buffer R was added to each blank control well. After mixing well, the plate was covered with a plate-sealing film and incubated at room temperature for 2 h. The plate was washed 4 times, before the detection antibody was added: 100 μL of biotinylated antibody working solution was added to each well. After mixing well, the plate was covered with a plate-sealing film and incubated at room temperature for 1 h. The plate was washed 4 additional times, before the enzyme was added: 100 μL of streptavidin-HRP working solution was added to each well. The plate was covered with a plate-sealing film and incubated at room temperature for 30 min. The plate was washed 4 additional times, before the color development: 100 μL of TMB liquid was added to each well, and the plate was incubated at room temperature (18-25° C.) in the dark for 25 min. Termination of reaction: The reaction was terminated by quickly adding a stop solution at 100 μL/well. Plate reading: The reading at wavelength 450 nm was detected using an M2e instrument within 10 min after the termination.

### Cell Viability Detection:

The chemiluminescent signal (RLU) was detected on the multimode microplate reader Envision according to the instructions of Celltiter-Glo.

Compound activity data analysis: The OD₄₅₀ values were converted to TNF-α and IFN-α contents by GraphPad Prism software analysis and fitted to give dose-response curves of the compounds. The EC₅₀ values of the compounds were calculated.

The results are shown in Table 2.

The results show that the small-molecule compounds of the present application could activate PBMC TLR7 and TLR8 receptors and induce the secretion of immune cytokines TNF-α and/or IFN-α, with activation activity superior to that of the controls VTX-2337 and R848.

TABLE 1

| Compound | hTLR7 EC₅₀ (μM) | hTLR8 EC₅₀ μM |
|---|---|---|
| R848 | 0.481 | 2.184 |
| VTX-2337 | >30 | 0.02113 |
| 10 | 6.259 | 0.03322 |
| 12 | 1.671 | 0.4318 |
| 13 | 14.36 | 0.06083 |
| 14 | 0.191 | 0.01163 |
| 15 | 0.712 | <0.0015 |
| 16 | 0.4338 | 0.05522 |
| 17 | 0.1925 | 0.1731 |
| 18 | 1.06 | 0.2603 |
| 19 | 0.1487 | 0.01131 |
| 20 | 0.07334 | 0.006525 |

TABLE 2

| Compound | TNF-α EC₅₀ (μM) | IFN-α EC₅₀ (μM) |
|---|---|---|
| R848 | 2.332 | 0.202 |
| VTX-2337 | 1.012 | >7.5 |
| 10 | 0.375 | 0.045 |
| 11 | 0.303 | 0.453 |
| 12 | 0.351 | 0.191 |
| 14 | 0.147 | 0.074 |
| 15 | 0.003 | 0.003 |
| 16 | 0.117 | 0.451 |
| 17 | 0.132 | 0.050 |

### Example 4

### Detection of Agonistic Activity of Antibody-Drug Conjugates on Whole Blood

In this example, the agonistic activity of the antibody-drug conjugates of the present application on human whole blood was assessed.

The antibody-drug conjugates of the present application was diluted to 200 nM with PBS, then serially 3-fold diluted to give a total of 5 concentrations, and added to 96-well cell plates, with 2 replicate wells set for each concentration. 0.2% DMSO was added to each negative control well, and 60 μM R848 was added to the positive control wells at 100 μL/well. Anticoagulated whole blood was added at 100 μL/well in the 96-well plates, and the cell plates were incubated at 37° C. with 5% CO₂ for 24 h. After the plates were centrifuged at 2000 G for 15 min at 4° C., the supernatants were collected to determine the concentrations of TNF-α and IFN-α.

TNF-α concentration assay was performed using a Dakewe TNF-α ELISA kit (Cat. No. 1117202). 100 μL of serially diluted cytokine standard was added to each standard well, 100 μL of 10-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

IFN-α concentration assay was performed using a Dakewe IFN-α ELISA kit (Cat. No. 1110012). 100 μL of serially diluted cytokine standard was added to each standard well, 100 μL of 2-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

The OD₄₅₀ values were converted to TNF-α and IFN-α contents by GraphPad Prism software analysis and fitted to give dose-response curves of the compounds. The EC₅₀ values of the compounds were calculated.

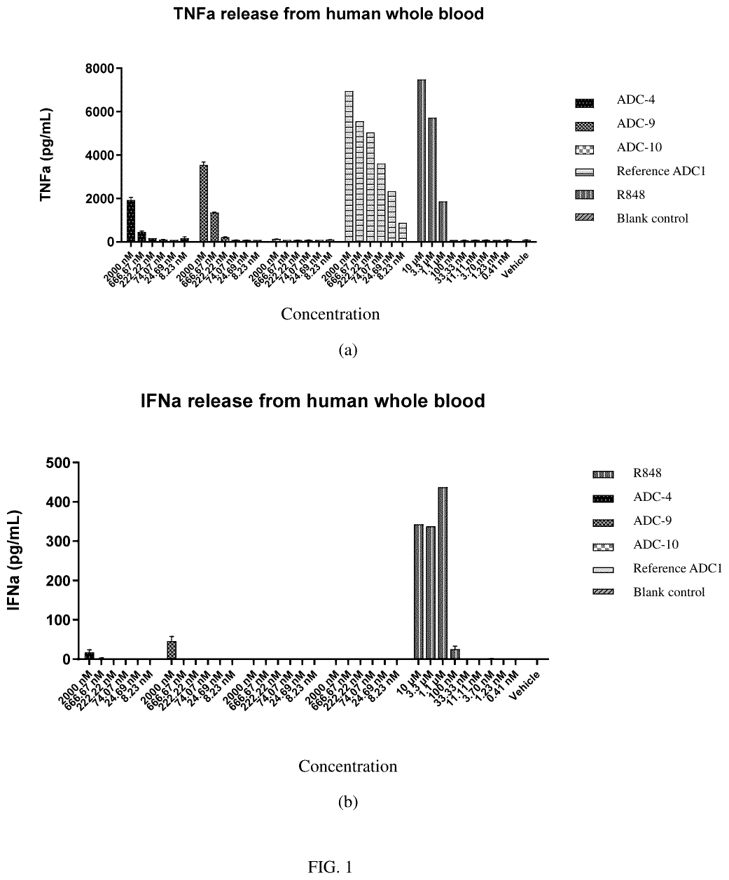

The results are shown in FIGS. 1(a) and (b).

The results show that the antibody-drug conjugates of the present application were active in human whole blood at high concentrations, but did not induce cytokine production at physiological blood concentrations, indicating that the antibody-drug conjugates of the present application had better safety and lower systemic toxicity, and were superior to the control ADC1.

### Example 5

Co-Incubation Experiments with Antibody-Drug Conjugates, Tumor Cells, and Human Peripheral Blood Mononuclear Cells (PBMCs)

The test antibody-drug conjugates were diluted to 400 nM with PBS buffer, then serially 4-fold diluted to give a total of 7 concentrations, and added to 96-well cell plates, with 2 replicate wells set for each concentration. 0.2% DMSO was added to each negative control well.

Pre-cultured NCI-N87, JIM-1 and MDA-MB-231 cells (cells purchased from the American Type Culture Collection) were digested and collected to give single-cell suspensions, which were then adjusted to 2×10⁶ cells/mL with the culture medium. Cryopreserved human PBMC cells were thawed, resuspended in a complete medium, and then counted, and the cell suspension was adjusted to 2×10⁷ cells/mL with the culture medium. The tumor cells and PBMCs were mixed at a volume ratio of 1:1, and then the mixed cells were added at 100 μL/500,000 cells/well. The cell plates were incubated at 37° C. with 5% CO₂ for 24 h, and then the supernatants were collected to determine the concentrations of TNF-α and IFN-α, as well as the activation of dendritic cells (DCs) and macrophages.

TNF-α concentration assay was performed using a Dakewe TNF-α ELISA kit (Cat. No. 1117202). 100 μL of serially diluted cytokine standard was added to each standard well, 100 μL of 10-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

IFN-α concentration assay was performed using a Dakewe IFN-α ELISA kit (Cat. No. 1110012). 100 μL of serially diluted cytokine standard was added to each standard well, 100 μL of 2-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

The activation of dendritic cells and macrophages was determined by using FACS to detect the activation of DC cells (HLA-DR+CD11c+) and macrophages (CD68+CD11b+) in PBMCs after co-incubation.

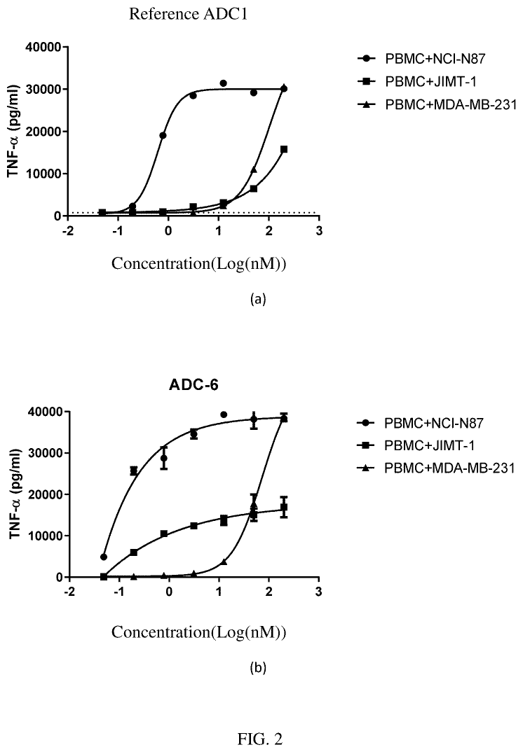

The results are shown in Table 3 and FIGS. 2(a) and (b).

The results show that the antibody-drug conjugate of the present application could induce the expression of cytokines, such as immune cytokines (e.g., TNF-α and/or IFN-α), in human PBMCs and the activation of immune cells (e.g., dendritic cells and macrophages), in the presence of tumor cells and was superior to the control ADC1.

TABLE 3

| | PBMC + NCI-N87 | | PBMC + NCI-N87 |
|---|---|---|---|
| Compound | EC50 (nM) | | Top (pg/mL) |
| Control ADC1 | 0.62 | nM | 30124 ± 367 |
| ADC-6 | 0.4 | nM | 38360 ± 648 |

### Example 6

Co-Incubation Experiment with Antibody-Drug Conjugate, Tumor Cells, and Human Peripheral Blood Mononuclear Cells (PBMCs)

The test antibody-drug conjugate was diluted to 100-400 nM with PBS buffer, then serially 4-fold diluted to give a total of 7 concentrations, and added to a 96-well cell plate, with 2 replicate wells set for each concentration. 0.2% DMSO was added to each negative control well. Pre-cultured HepG2 cells were digested and collected to give a single-cell suspension, which was then adjusted to 2×10⁶ cells/mL with the culture medium. Cryopreserved human PBMC cells were thawed, resuspended in a complete medium, and then counted, and the cell suspension was adjusted to 2×10⁷ cells/mL with the culture medium. The tumor cells and PBMCs were mixed at a volume ratio of 1:1, and then the mixed cells were added at 100 μL/500,000 cells/well. The cell plate was incubated at 37° C. with 5% CO₂ for 18 h, and then the supernatant was collected to determine the concentrations of TNF-α and IFN-α, as well as to detect the activation of dendritic cells (DCs) and macrophages.

TNF-α concentration assay was performed using a Dakewe TNF-α ELISA kit (Cat. No. 1117202). 100 μL of serially diluted cytokine standard was added to each standard well, 100 μL of 10-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

IFN-α concentration assay was performed using a Dakewe IFN-α ELISA kit (Cat. No. 1110012). 100 μL of serially diluted cytokine standard was added to each standard well, 100 μL of 2-fold diluted test sample was added to each sample well, and 100 μL of 1× dilution buffer R was added to each blank control well. 50 μL of biotinylated antibody working solution was added to each well. After mixing well, the plate was covered with a plate-sealing film and incubated at room temperature for 3 h. The plate was washed 3 times: the liquid in each well was removed, and 300 μL of 1× washing buffer working solution was added to each well; after 1 minute of residence, the liquid in each well was discarded. This procedure was repeated 3 times. 100 μL of streptavidin-HRP working solution was added to each well. The plate was covered with a plate-sealing film and incubated at room temperature for 20 min. The plate was washed 3 times, 100 μL of TMB liquid was added to each well, and the plate was incubated at room temperature (18-25° C.) in the dark for 25 min. The reaction was terminated by quickly adding a stop solution at 100 μL/well. The reading at wavelength 450 nm was detected using an M2e instrument within 10 min after the termination.

The activation of dendritic cells and macrophages was determined by using FACS to detect the activation of DC cells (HLA-DR+CD11c+) and macrophages (CD68+CD11b+) in PBMCs after co-incubation.

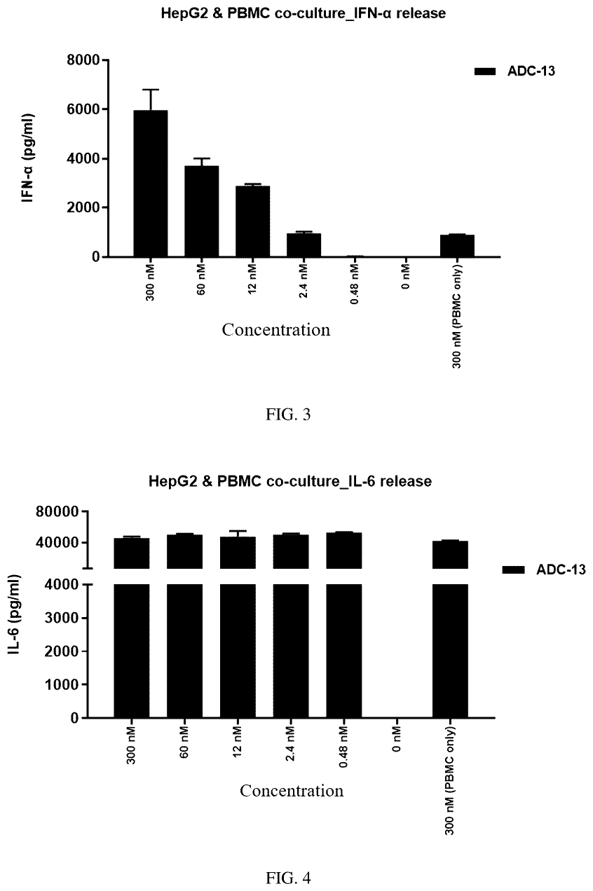

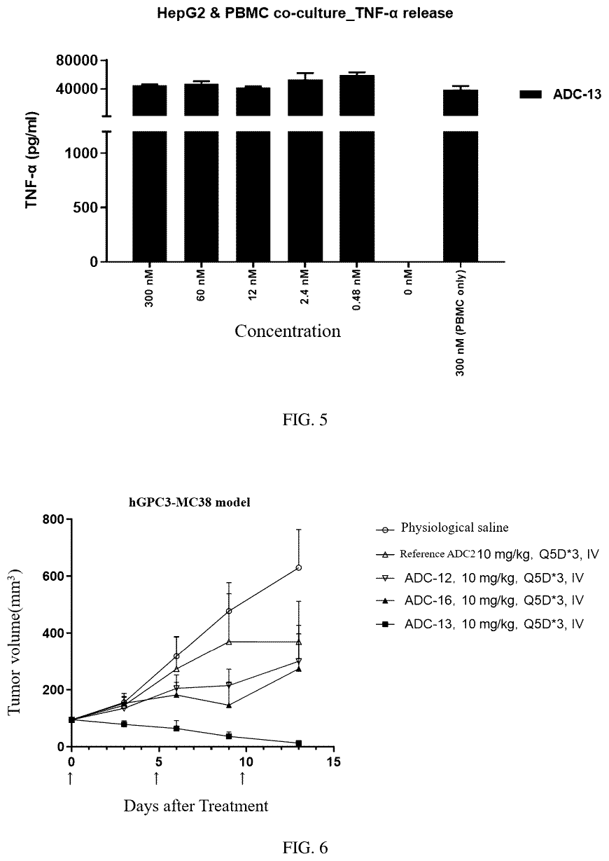

The results are shown in FIGS. 3, 4 and 5.

The results show that the antibody-drug conjugate of the present application could induce a significant increase in the expression of cytokines, such as immune cytokines (e.g., TNF-α and/or IFN-α), in human PBMCs, in the presence of tumor cells. The antibody-drug conjugate of the present application could activate immune cells, such as dendritic cells and macrophages, so that the activity of the cells was significantly improved.

### Example 7

Establishment of hGPC3-MC38 Colon Cancer Animal Model Using Humanized Mice with hTLR8 Gene Knock-In and Assay of In Vivo Efficacy of Drugs

hGPC3-MC38 cells are a cell line that is constructed by a transgenic technique of site-directed modification to stably express human GPC3 protein on the surface of mouse colon cancer cells MC38. Flow cytometry (FACS) using anti-human GPC3 antibodies has confirmed the high expression of human hGPC3 (hGPC3) protein in this cell line.

hTLR8-C57BL/6 mice (hTLR8 humanized mice) are C57BL/6 mice transgenic for the human TLR8 (hTLR8) gene, purchased from Biocytogen; spleen cells from wild-type C57BL/6 and hTLR8 humanized C57BL/6 mice were collected and analyzed using flow cytometry with species-specific anti-TLR8 antibodies; the results show that hTLR8 was detected on the surface of DC cells in the hTLR8-C57BL/6 mice, while hTLR8 expression was undetectable in the wild-type mice, confirming the knock-in and protein expression of the hTLR8 gene. Spleen cells collected from hTLR8-C57BL/6 mice were stimulated with the TLR8 agonists TLR8-506 and GS-9688 and subjected to ELISA; the results showed an increase in the secretion of TNFα and IFNγ, confirming the functionality of hTLR8 in hTLR8-C57BL/6 mice (as TLR8 was not functional in the mice).

### In Vivo Efficacy Experiment 1:

hGPC3-MC38 cells resuspended in PBS were subcutaneously inoculated into the right anterior scapula of 8-week-old female hTLR8-C57BL/6 mice at 1×10⁶ cells/0.1 mL/mouse. When the average tumor volume reached about 120 mm³, the mice were selected and grouped based on the tumor volume, with 6-8 mice in each group. The antibody-drug conjugates of the present application were diluted to 1-5 mg/mL with PBS buffer and administered by intravenous (i.v) injection at a dose of 10 mg/kg starting on the day of grouping (day 0), with a dosing frequency of once every 5 days for 3 consecutive administrations. Tumor inhibition rates were calculated by measuring tumor volumes. Tumor inhibition rate (TGI %)=100%−(tumor volume of the treatment group on the day of measurement−tumor volume of the treatment group on day 0)/(tumor volume of the control group on the day of measurement−tumor volume of the control group on day 0). In the case of tumor regression, tumor inhibition rate=[1−(tumor volume of the treatment group on the day of measurement÷tumor volume of the treatment group on day 0)÷(tumor volume of the control group on the day of measurement÷tumor volume of the control group on day 0)]÷[1−(tumor volume of the control group on day 0÷tumor volume of the control group on the day of measurement)]×100%. The results are shown in Table 4 and FIG. 6.

TABLE 4

| | Tumor volume on | Tumor volume on | |
|---|---|---|---|
| | day 0 (mm³) | day 13 (mm³) | TGI |
| Normal saline | 94.4 ± 10.5 | 629.8 ± 299.6 | |
| (control group) | | | |
| Control ADC2 | 95.3 ± 10.0 | 369.2 ± 349.6 | 36.3% |
| ADC-12 | 94.4 ± 12.4 | 299.8 ± 238.8 | 52.2% |
| ADC-16 | 95.0 ± 14.4 | 273.8 ± 343.0 | 58.4% |
| ADC-13 | 95.0 ± 12.6 | 12.8 ± 20.8 | 119.1% |

The results show that the antibody-drug conjugates of the present application could effectively inhibit tumor cell growth in vivo, and were superior to the control ADC2.

### In Vivo Efficacy Experiment 2:

hGPC3-MC38 cells resuspended in PBS were subcutaneously inoculated into the right anterior scapula of 8-week-old female hTLR8-C57BL/6 mice at 1×10⁶ cells/0.1 mL/mouse. When the average tumor volume reached about 120 mm³, the mice were selected and grouped based on the tumor volume, with 6-8 mice in each group. The antibody-drug conjugate of the present application was diluted to 1-5 mg/mL with PBS buffer and administered by intravenous injection (10 mg/kg) or subcutaneous injection (15 mg/kg) starting on the day of grouping (day 0), with a dosing frequency of once every week (QW) for 2 consecutive administrations.

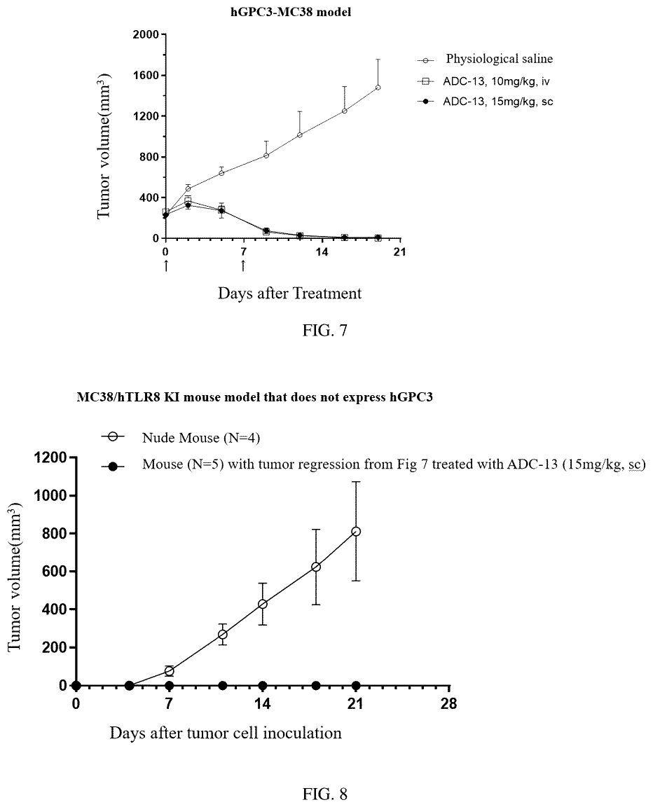

The results are shown in Table 5 and FIG. 7.

TABLE 5

| | Tumor volume on | Tumor volume on | |
|---|---|---|---|
| | day 0 (mm³) | day 19 (mm³) | TGI |
| Normal saline | 227.8 ± 22.4 | 1479.2 ± 615.1 | |
| (control group) | | | |
| ADC-13, iv (10 | 259.4 ± 36.5 | 3.75 ± 7.5 | 117.9% |
| mg/kg) | | | |
| ADC-13, sc | 232.0 ± 41.4 | 11.2 ± 25.0 | 117.3% |
| (15 mg/kg) | | | |

The results show that both intravenous and subcutaneous injections of the antibody-drug conjugate of the present application could effectively inhibit tumor cell growth in vivo.

### Tumor Rechallenge Experiment:

Mice with complete tumor regression and hTLR8 humanized mice that had not been treated with drugs or inoculated with tumors (naïve) were selected for the rechallenge experiment: WT MC38 cells suspended in PBS were subcutaneously inoculated into the left anterior scapula of the hTLR8 humanized mice at 5×10⁵ cells/0.1 mL/mouse. The day of inoculation was designated as day 0, and tumor volume was measured every 2 days thereafter.

The results are shown in FIG. 8.

The results of the tumor rechallenge experiment show that the antibody-drug conjugate of the present application could induce a long-term immune memory effect, exhibiting a long-term effective inhibition effect on tumor cell growth.

The foregoing detailed description is provided by way of illustration and instances, and is not intended to limit the scope of the appended claims. Various modifications of the embodiments currently enumerated in the present application will be apparent to those of ordinary skill in the art and are intended to be within the scope of the appended claims and their equivalents.

Sequence list of the present application (all sequences are amino acid sequences; for the definition/description of the sequences, refer to the previous sections herein):

| Sequence No. | Sequence |
|---|---|
| SEQ ID NO: 1 | RASQDVNTAVA |
| SEQ ID NO: 2 | KASQDVSIGVA |
| SEQ ID NO: 3 | KASQDVSIAVA |
| SEQ ID NO: 4 | RSSQSLVHSNRNTYLH |
| SEQ ID NO: 5 | SASFLY |
| SEQ ID NO: 6 | SASYRYT |
| SEQ ID NO: 7 | SASYRYT |
| SEQ ID NO: 8 | KVSNRFS |
| SEQ ID NO: 9 | QQHYTTPP |
| SEQ ID NO: 10 | QQYYIYPY |
| SEQ ID NO: 11 | QQHYITPLT |
| SEQ ID NO: 12 | SQNTHVPPT |
| SEQ ID NO: 13 | GFNIKDTYIH |
| SEQ ID NO: 14 | GFTFTDYTMD |
| SEQ ID NO: 15 | NYGMN |
| SEQ ID NO: 16 | DYEMH |
| SEQ ID NO: 17 | ARIYPTNGYTRYADSVKG |
| SEQ ID NO: 18 | ADVNPNSGGSIYNQRFKG |
| SEQ ID NO: 19 | WINTYTGEPTYTDDFKG |
| SEQ ID NO: 20 | ALDPKTGDTAYSQKFKG |
| SEQ ID NO: 21 | SRWGGDGFYAMDY |
| SEQ ID NO: 22 | ARNLGPSFYFDY |
| SEQ ID NO: 23 | GGFGSSYWYFDV |
| SEQ ID NO: 24 | FYSYTY |
| SEQ ID NO: 25 | DIQMTQSPSSLSASVGDRVTITCRASQDVNTAVAWYQQKPGKAPKLLIYSASF |
| | LYSGVPSRFSGSRSGTDFTLTISSLQPEDFATYYCQQHYTTPPTFGQGTKVEIK |
| SEQ ID NO: 26 | DIQMTQSPSSLSASVGDRVTITCKASQDVSIGVAWYQQKPGKAPKLLIYSASY |
| | RYTGVPSRFSGSGSGTDFTLTISSLQPEDFATYYCQQYYIYPYTFGQGTKVEIK |
| SEQ ID NO: 27 | DIQLTQSPSSLSASVGDRVSITCKASQDVSIAVAWYQQKPGKAPKLLIYSASYR |
| | YTGVPDRFSGSGSGTDFTLTISSLQPEDFAVYYCQQHYITPLTFGAGTKVEIK |
| SEQ ID NO: 28 | DVVMTQSPLSLPVTPGEPASISCRSSQSLVHSNRNTYLHWYLQKPGQSPQLLI |
| | YKVSNRFSGVPDRFSGSGSGTDFTLKISRVEAEDVGVYYCSQNTHVPPTFGQ |
| | GTKLEIK |
| SEQ ID NO: 29 | EVOLVESGGGLVQPGGSLRLSCAASGFNIKDTYIHWVRQAPGKGLEWVARIY |
| | PTNGYTRYADSVKGRFTISADTSKNTAYLQMNSLRAEDTAVYYCSRWGGDG |
| | FYAMDYWGQGTLVTVSS |
| SEQ ID NO: 30 | EVOLVESGGGLVQPGGSLRLSCAASGFTFTDYTMDWVRQAPGKGLEWVAD |
| | VNPNSGGSIYNQRFKGRFTLSVDRSKNTLYLQMNSLRAEDTAVYYCARNLGP |
| | SFYFDYWGQGTLVTVSS |
| SEQ ID NO: 31 | QVQLQQSGSELKKPGASVKVSCKASGYTFTNYGMNWVKQAPGQGLKWMG |
| | WINTYTGEPTYTDDFKGRFAFSLDTSVSTAYLQISSLKADDTAVYFCARGGFG |
| | SSYWYFDVWGQGSLVTVSS |
| SEQ ID NO: 32 | QVQLVQSGAEVKKPGASVKVSCKASGYTFTDYEMHWVRQAPGQGLEWMG |
| | ALDPKTGDTAYSQKFKGRVTLTADKSTSTAYMELSSLTSEDTAVYYCTRFYSY |
| | TYWGQGTLVTVSS |
| SEQ ID NO: 33 | DIQMTQSPSSLSASVGDRVTITCRASQDVNTAVAWYQQKPGKAPKLLIYSASF |
| | LYSGVPSRFSGSRSGTDFTLTISSLOPEDFATYYCQQHYTTPPTFGQGTKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC |
| SEQ ID NO: 34 | DIQMTQSPSSLSASVGDRVTITCKASQDVSIGVAWYQQKPGKAPKLLIYSASY |
| | RYTGVPSRFSGSGSGTDFTLTISSLQPEDFATYYCQQYYIYPYTFGQGTKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC |
| SEQ ID NO: 35 | DIQLTQSPSSLSASVGDRVSITCKASQDVSIAVAWYQQKPGKAPKLLIYSASYR |
| | YTGVPDRFSGSGSGTDFTLTISSLQPEDFAVYYCQQHYITPLTFGAGTKVEIKR |
| | TVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRG |
| | EC |
| SEQ ID NO: 36 | DVVMTQSPLSLPVTPGEPASISCRSSQSLVHSNRNTYLHWYLQKPGQSPQLLI |
| | YKVSNRFSGVPDRFSGSGSGTDFTLKISRVEAEDVGVYYCSQNTHVPPTFGQ |
| | GTKLEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLSSP |
| | VTKSFNRGEC |
| SEQ ID NO: 37 | EVQLVESGGGLVQPGGSLRLSCAASGFNIKDTYIHWVRQAPGKGLEWVARIY |
| | PTNGYTRYADSVKGRFTISADTSKNTAYLQMNSLRAEDTAVYYCSRWGGDG |
| | FYAMDYWGQGTLVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNHK |
| | PSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| SEQ ID NO: 38 | EVQLVESGGGLVQPGGSLRLSCAASGFTFTDYTMDWVRQAPGKGLEWVAD |
| | VNPNSGGSIYNQRFKGRFTLSVDRSKNTLYLQMNSLRAEDTAVYYCARNLGP |
| | SFYFDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPG |
| SEQ ID NO: 39 | QVQLQQSGSELKKPGASVKVSCKASGYTFTNYGMNWVKQAPGQGLKWMG |
| | WINTYTGEPTYTDDFKGRFAFSLDTSVSTAYLQISSLKADDTAVYFCARGGFG |
| | SSYWYFDVWGQGSLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| SEQ ID NO: 40 | QVQLVQSGAEVKKPGASVKVSCKASGYTFTDYEMHWVRQAPGQGLEWMG |
| | ALDPKTGDTAYSQKFKGRVTLTADKSTSTAYMELSSLTSEDTAVYYCTRFYSY |
| | TYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

1. A ligand-drug conjugate, or an isomer thereof, or a mixture thereof, or a pharmaceutically acceptable salt thereof, wherein the ligand-drug conjugate comprises a structure represented by formula (II-2A): 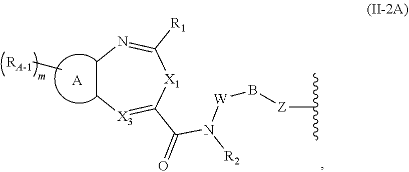
wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl;
ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl;
when R_{A-1} comprises methylene units, the methylene units of R_{A-1} are each independently unreplaced, or are each independently replaced by any structure;
m is selected from 1, 2, or 3;
the wavy line [symbol] in the general formula represents being directly linked to a ligand via the nitrogen atom on the Z group, or being linked to the ligand via a linker unit.

2. The ligand-drug conjugate, or the isomer thereof, or the mixture thereof, or the pharmaceutically acceptable salt thereof according to claim 1, wherein the ligand-drug conjugate comprises a structure represented by formula (II-2B): 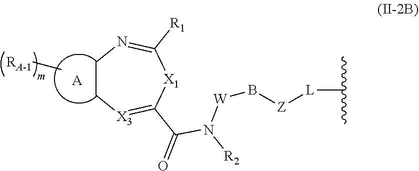
wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl;
ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl;
when R_{A-1} comprises methylene units, the methylene units of R_{A-1} are each independently unreplaced, or are each independently replaced by any structure;
m is selected from 1, 2, or 3;
the wavy line [symbol] in the general formula represents being linked to the ligand via the nitrogen atom or the carbon atom on the L group;
L is a linker unit.

3. The ligand-drug conjugate, or the isomer thereof, or the mixture thereof, or the pharmaceutically acceptable salt thereof according to claim 1, wherein the ligand-drug conjugate has a structure represented by formula (II-2C): 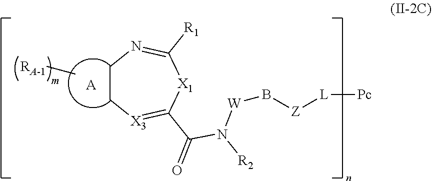
wherein
R₁ is optionally substituted amino;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl;
ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl;
when R_{A-1} comprises methylene units, the methylene units of R_{A-1} are each independently unreplaced, or are each independently replaced by any structure;
m is selected from 1, 2, or 3;
L is a linker unit;
n is an integer or a decimal from 1 to 10;
Pc is a ligand.

4. The ligand-drug conjugate, or the isomer thereof, or the mixture thereof, or the pharmaceutically acceptable salt thereof according to any one of claims 1-3, wherein R₁ is —NH₂.

5. The ligand-drug conjugate, or the isomer thereof, or the mixture thereof, or the pharmaceutically acceptable salt thereof according to any one of claims 1-3, wherein X₁ is selected from —CH₂— or

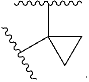 [SMILES: *C1(*)CC1]

6. The ligand-drug conjugate, or the isomer thereof, or the mixture thereof, or the pharmaceutically acceptable salt thereof according to any one of claims 1-3, wherein X₃ is selected from —CH═ or —C(CH₃)=.

7. The compound or the conjugate thereof, or the isomer thereof, or the mixture thereof, or the pharmaceutically acceptable salt thereof according to any one of claims 1-3, wherein
R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂-2, each R₂₋₂ being independently selected from: hydrogen, halogen, hydroxy, or amino;
the methylene units of R₂ are each independently unreplaced, replaced by —O—, or replaced by —N(R₂₋₃)—, R₂₋₃ being selected from: hydrogen or C₁-C₃ alkyl.

8. The ligand-drug conjugate, or the isomer thereof, or the mixture thereof, or the pharmaceutically acceptable salt thereof according to claim 7, wherein each R₂₋₁ is independently selected from: hydrogen, fluorine, chlorine, bromine, iodine, hydroxy, amino, —CN, —CH₃, —CH₂F, —CHF₂, —CF₃, —CH₂CH₃, or

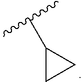 [SMILES: *C1CC1]

9. The ligand-drug conjugate, or the isomer thereof, or the mixture thereof, or the pharmaceutically acceptable salt thereof according to any one of claims 1-8, wherein R₂ is selected from 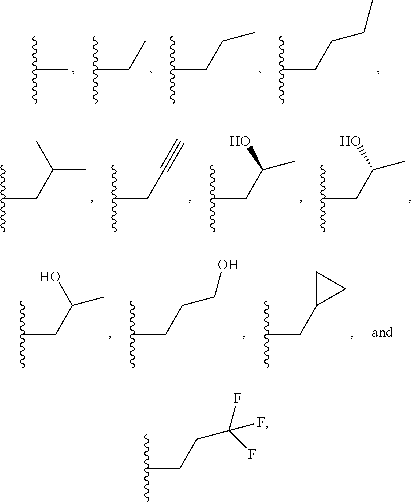
wherein the methylene units of R₂ are each independently unreplaced, replaced by —O—, or replaced by —NH—.

10. The ligand-drug conjugate, or the isomer thereof, or the mixture thereof, or the pharmaceutically acceptable salt thereof according to any one of claims 1-9, wherein R₂ is selected from:

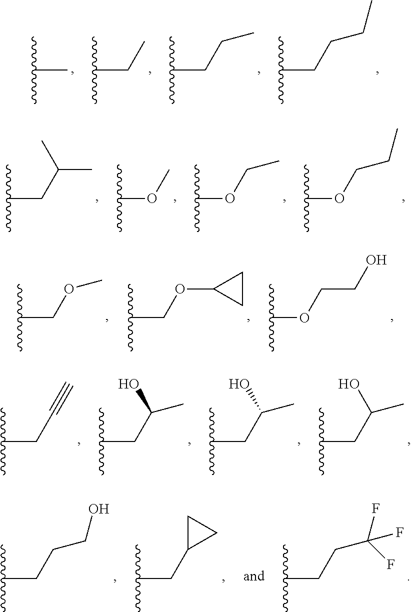

11. The ligand-drug conjugate, or the isomer thereof, or the mixture thereof, or the pharmaceutically acceptable salt thereof according to any one of claims 1-3, wherein
W is selected from C₁-C₆ alkylene optionally substituted with 1, 2, or 3 R_{w-1}; each R_{w-1} is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2}, each R_{w-2} being independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl.

12. The ligand-drug conjugate, or the isomer thereof, or the mixture thereof, or the pharmaceutically acceptable salt thereof according to claim 11, wherein each R_{w-1} is independently selected from: hydrogen, fluorine, chlorine, bromine, iodine, hydroxy, amino, —CN, —CH₃, —CH₂F, —CHF₂, —CF₃, —CH₂CH₃, or

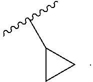 [SMILES: *C1CC1]

13. The ligand-drug conjugate, or the isomer thereof, or the mixture thereof, or the pharmaceutically acceptable salt thereof according to claim 12, wherein W is selected from 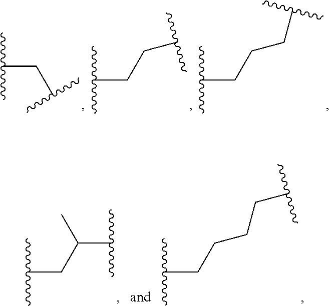
wherein the methylene units of W are each independently unreplaced, replaced by —O—, or replaced by —NH—.

14. The ligand-drug conjugate, or the isomer thereof, or the mixture thereof, or the pharmaceutically acceptable salt thereof according to claim 13, wherein W is selected from

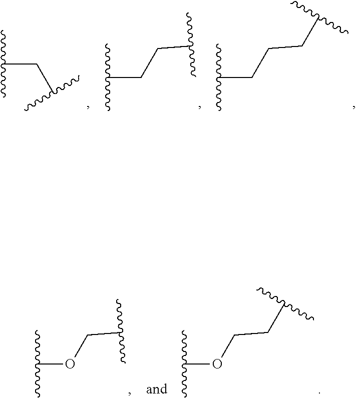

15. The ligand-drug conjugate, or the isomer thereof, or the mixture thereof, or the pharmaceutically acceptable salt thereof according to any one of claims 1-3, wherein B is selected from phenyl and pyridinyl, wherein the phenyl and the pyridinyl are each independently optionally substituted with 1, 2, or 3 R_{B-1}, each R_{B-1} being independently selected from hydrogen, halogen, hydroxy, amino, C₁-C₃ alkyl, and C₁-C₃ alkoxy.

16. The ligand-drug conjugate, or the isomer thereof, or the mixture thereof, or the pharmaceutically acceptable salt thereof according to any one of claims 1-15, wherein B is selected from 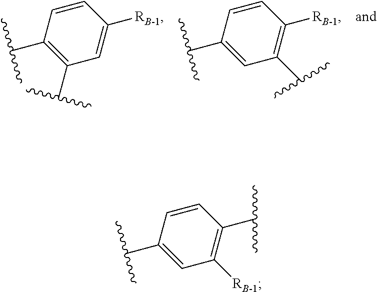
preferably, B is selected from

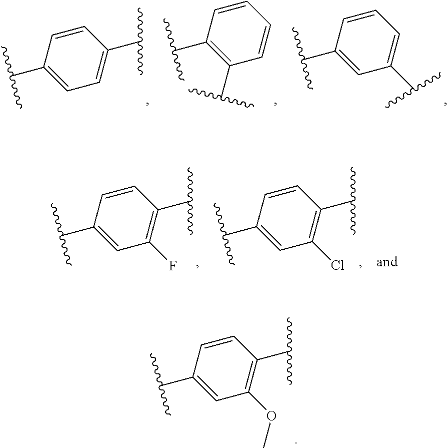

17. The ligand-drug conjugate, or the isomer thereof, or the mixture thereof, or the pharmaceutically acceptable salt thereof according to any one of claims 1-3, wherein A is selected from: phenyl, pyridinyl, pyrrolyl, thienyl, furanyl, pyridazinyl, pyrimidinyl, and pyrazinyl.

18. The ligand-drug conjugate, or the isomer thereof, or the mixture thereof, or the pharmaceutically acceptable salt thereof according to any one of claims 1-17, wherein A is selected from phenyl and pyridinyl.

19. The ligand-drug conjugate, or the isomer thereof, or the mixture thereof, or the pharmaceutically acceptable salt thereof according to any one of claims 1-3, wherein
R_{A-1} is R_{A2-1}-Lx-;
Lx is selected from: —(CH₂)ₜ—, —(CH₂)ₜO—, —O(CH₂)ₜ—, —(CH₂)ₜN(R_{L-1})—, —(CH₂)ₜS—, —(CH₂)ₜC(═O)—, —(CH₂)ₜN(R_{L-1})C(═O)—, 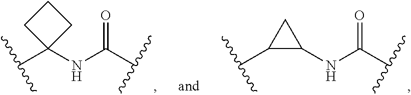 [SMILES: *C(=O)NC1(*)CCC1 ; *C(=O)NC1CC1*]
wherein R_{L-1} is selected from H or C₁-C₃ alkyl, and t is selected from 0, 1, 2, or 3;
R_{A2-1} is selected from hydrogen, halogen, hydroxy, amino, cyano, C₁-C₆ alkyl, alcyl, aliphatic heterocyclyl, aryl, and heteroaryl, wherein the C₁-C₆ alkyl, the alcyl, the aliphatic heterocyclyl, the aryl, and the heteroaryl are each independently optionally substituted with 1, 2, or 3 R_{A2-2}; R_{A2-2} is selected from hydrogen, halogen, hydroxy, amino, and C₁-C₆ alkyl optionally substituted with 1, 2, or 3 R_{A2-3}; each R_{A2-3} is independently selected from: hydrogen, halogen, hydroxy, amino, and C₁-C₃ alkyl;
any methylene unit of R_{A2-1} may be replaced by the following structure: —O—, —S—, —S(═O)₂—, —NH—, —CO—, 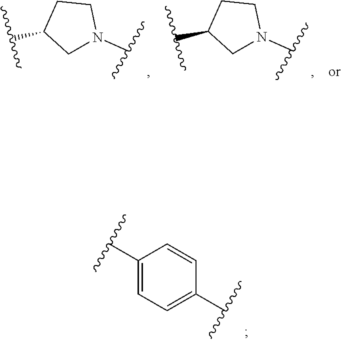 [SMILES: *[C@@H]1CCN(*)C1 ; *[C@H]1CCN(*)C1 ; *c1ccc(*)cc1]
m is selected from 1, 2, or 3.

20. The ligand-drug conjugate, or the isomer thereof, or the mixture thereof, or the pharmaceutically acceptable salt thereof according to claim 19, wherein Lx is a single bond or —NHC(═O)—.

21. The ligand-drug conjugate, or the isomer thereof, or the mixture thereof, or the pharmaceutically acceptable salt thereof according to claim 19 or 20, wherein each R_{A2-1} is independently selected from hydrogen, halogen, hydroxy, amino, cyano, or C₁-C₆ alkyl, 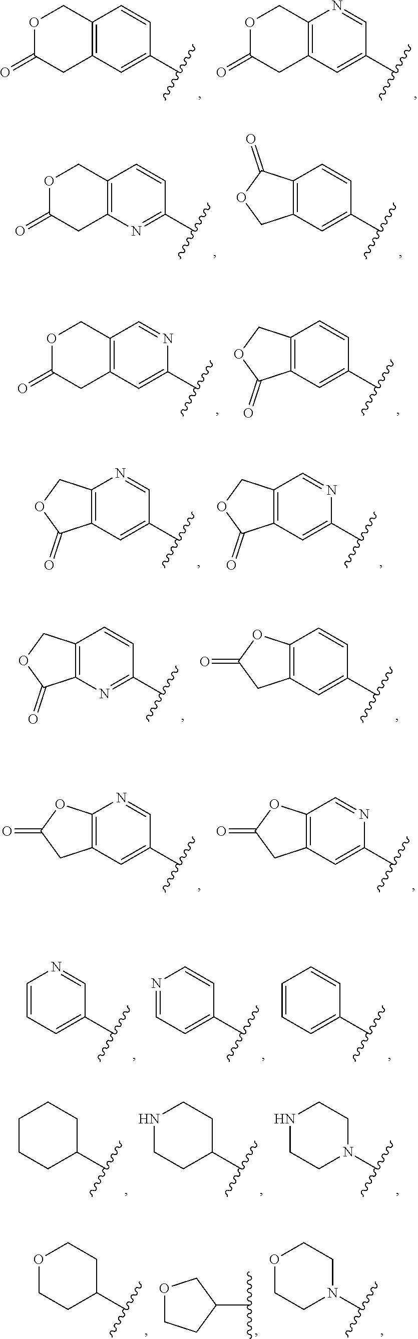 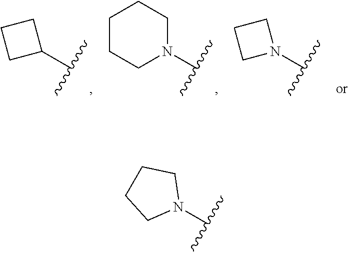
optionally substituted with 1, 2, or 3 R_{A2-2}, wherein any methylene unit of R_{A2-1} can be replaced by the following structure: —O—, —S—, —S(═O)—, —S(═O)₂—, —NH—, —C(O)—,

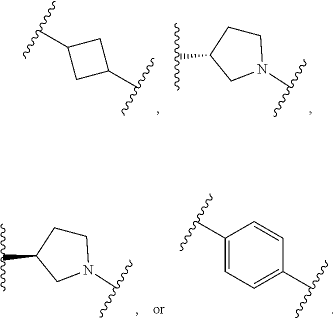 [SMILES: *C1CC(*)C1 ; *[C@@H]1CCN(*)C1 ; *[C@H]1CCN(*)C1 ; *c1ccc(*)cc1]

22. The ligand-drug conjugate, or the isomer thereof, or the mixture thereof, or the pharmaceutically acceptable salt thereof according to any one of claims 1-21, wherein R_{A-1} is selected from: hydrogen, fluorine, chlorine, bromine, iodine, hydroxy, amino, cyano, sulfhydryl, —CH₃, —CH(═O), —C(═O)OH, —OCH(═O), —SH(═O), —SH(═O)₂, —CH₂CH₃, —CH₂OH, —OCH₃, —NHCH₃, —CH₂NH₂, —S(═O)₂NH₂, —NHSH(═O)₂, —SCH₃, —CH₂SH, —S(═O)CH₃, —CH₂SH(═O), —S(═O)₂CH₃, —CH₂SH(═O)₂, —NHCH(═O), —C(═O)NH₂, —CH₂CH(═O), —CH₂NHCH(═O), —C(═O)NHCH₃,

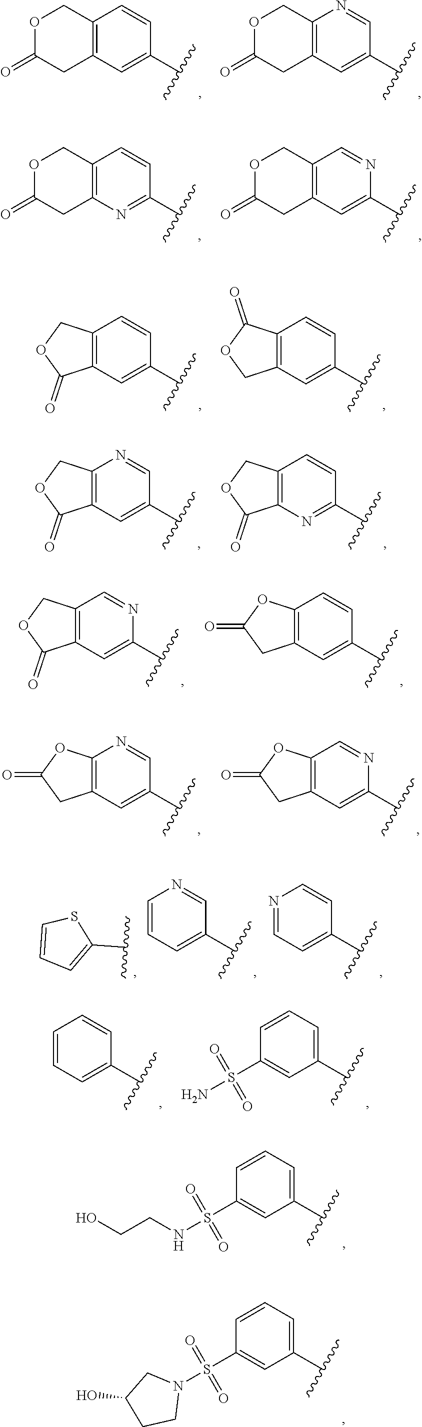

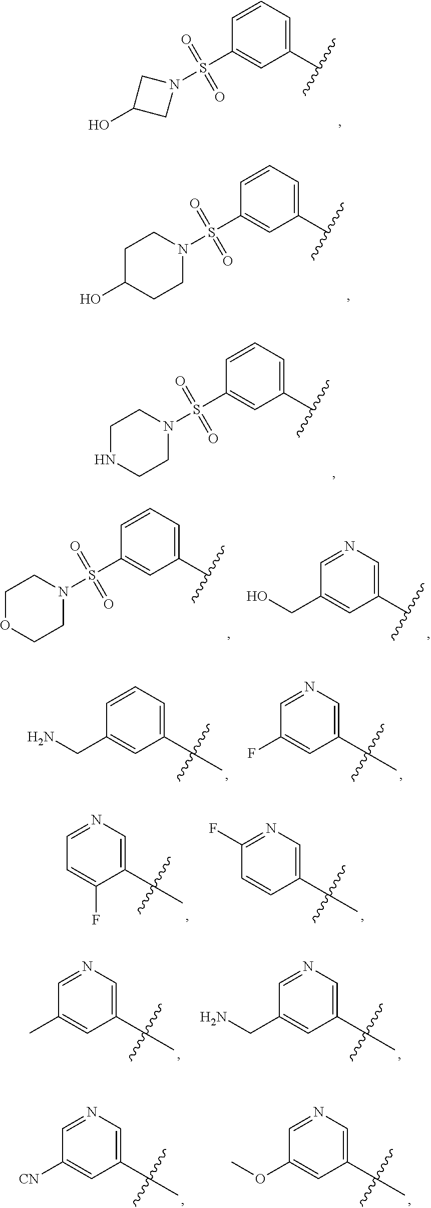

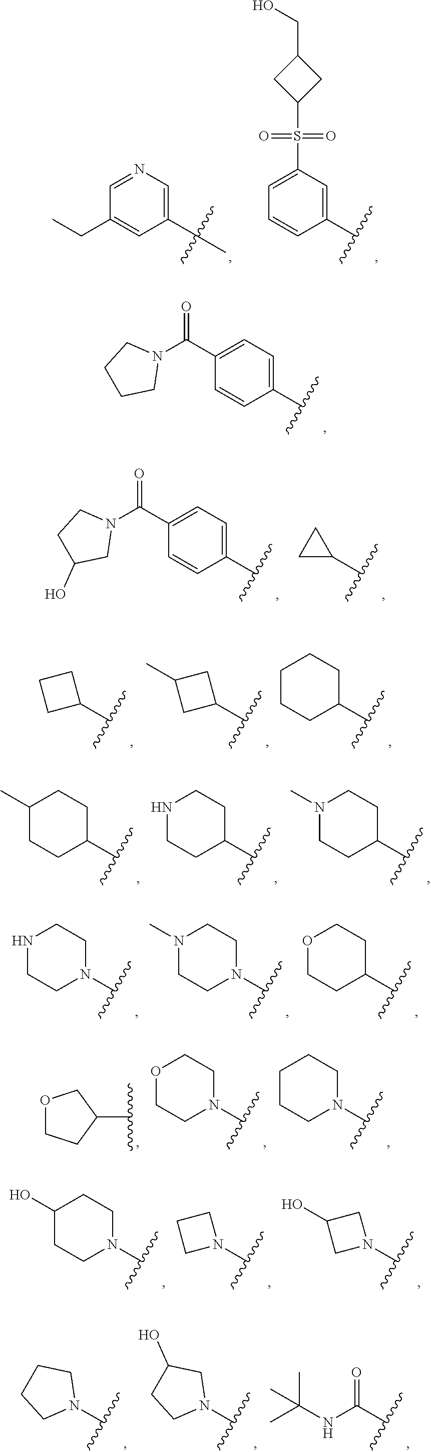

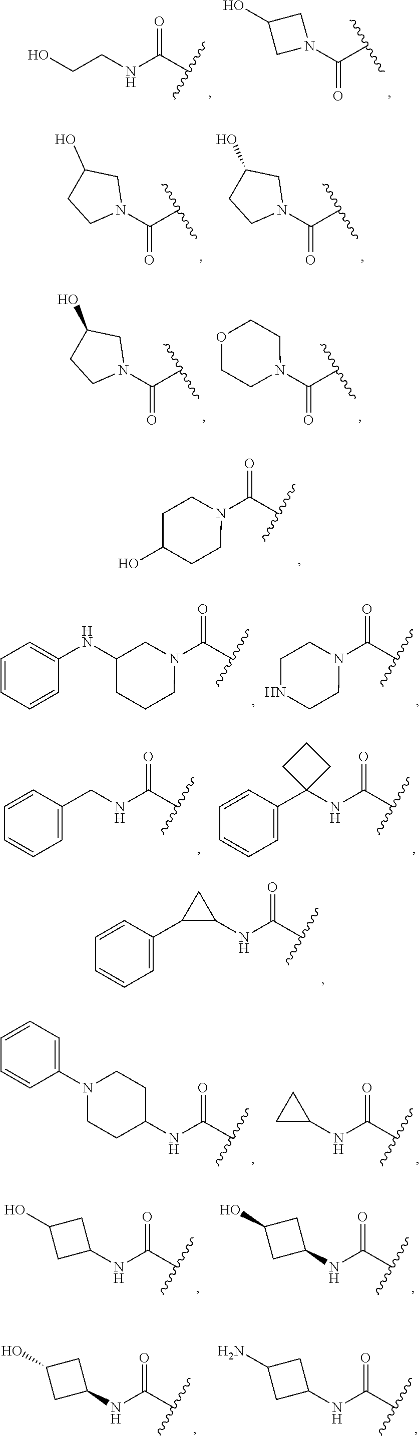

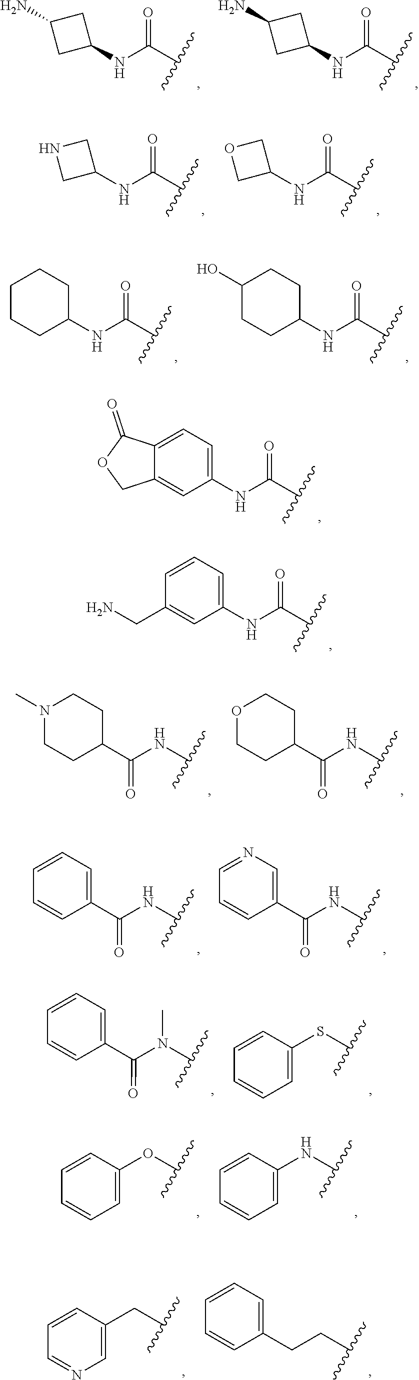

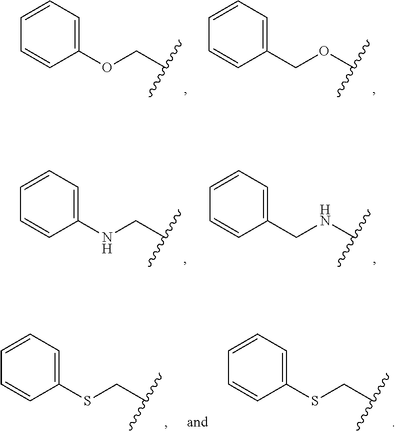 [SMILES: *COc1ccccc1 ; *OCc1ccccc1 ; *CNc1ccccc1 ; *NCc1ccccc1 ; *CSc1ccccc1 ; *CSc1ccccc1]

23. The ligand-drug conjugate, or the isomer thereof, or the mixture thereof, or the pharmaceutically acceptable salt thereof according to any one of claims 1-22, wherein R_{A-1} is R_{A2-1}-Lx-, wherein Lx is a single bond or an amide bond, and each R_{A2-1} is independently selected from phenyl and pyridinyl optionally substituted with 1, 2, or 3 R_{A2-2}; R_{A2-2} is selected from hydrogen, halogen, hydroxy, amino, and C₁-C₆ alkyl optionally substituted with 1, 2, or 3 R_{A2-3}; each R_{A2-3} is independently selected from: hydrogen, halogen, hydroxy, and amino;
preferably, R_{A-1} is selected from: 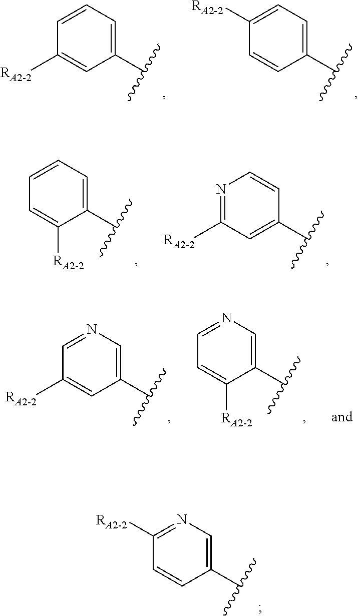
more preferably, R_{A-1} is selected from:

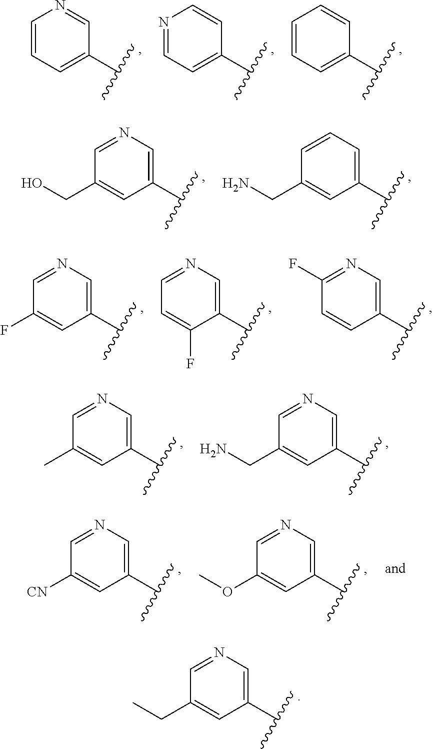

24. The ligand-drug conjugate, or the isomer thereof, or the mixture thereof, or the pharmaceutically acceptable salt thereof according to any one of claims 1-23, wherein Z is selected from —N(R_{Z-1})—, wherein R_{Z-1} is selected from H or C₁-C₆ alkyl optionally substituted with 1, 2, or 3 fluorine, chlorine, bromine, iodine, hydroxy, or amino substituents; preferably, R_{Z-1} is selected from H, —CH₃, —CH₂F, —CHF₂, —CF₃, and —CH₂CH₃.

25. The ligand-drug conjugate, or the isomer thereof, or the mixture thereof, or the pharmaceutically acceptable salt thereof according to any one of claims 1-24, wherein the structural unit 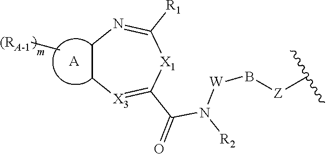
is selected from: 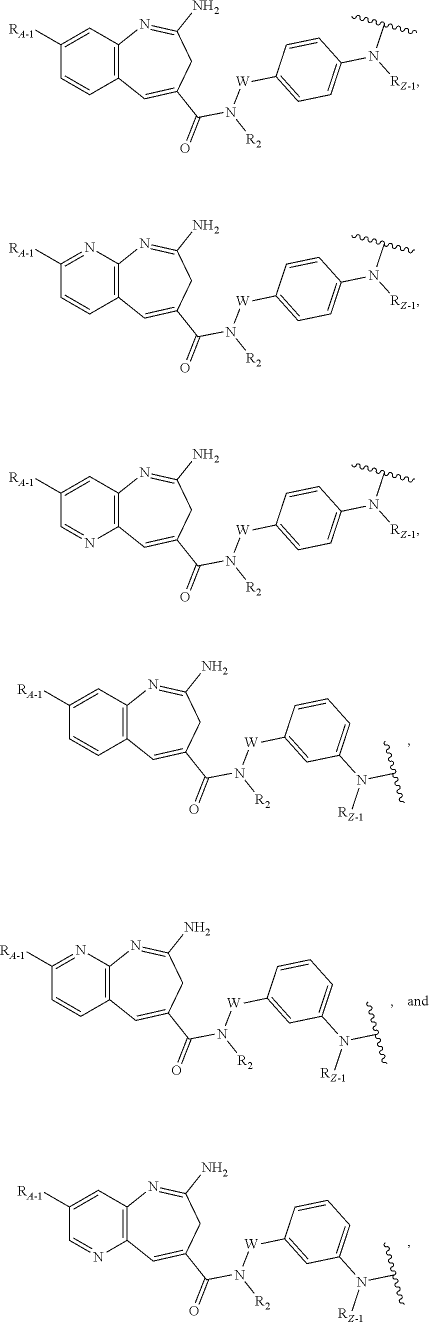
wherein
each R₂ is as defined in any one of claims 7-10;
R_{Z-1} is as defined in claim 1 or 24;
each W is as defined in any one of claims 11-14;
each R_{A-1} is as defined in any one of claims 19-23.

26. The ligand-drug conjugate, or the isomer thereof, or the mixture thereof, or the pharmaceutically acceptable salt thereof according to any one of claims 2-25, wherein the linker unit L is -Tr-L₃-L₂-L_{1c}-, wherein
Tr is selected from 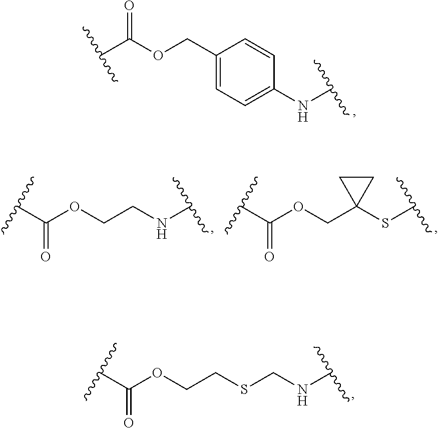 [SMILES: *Nc1ccc(COC(*)=O)cc1 ; *NCCOC(*)=O ; *SC1(COC(*)=O)CC1 ; *NCSCCOC(*)=O]
and a single bond;
L_{1c} is selected from 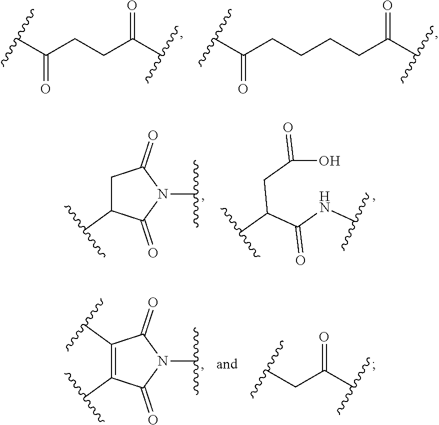 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 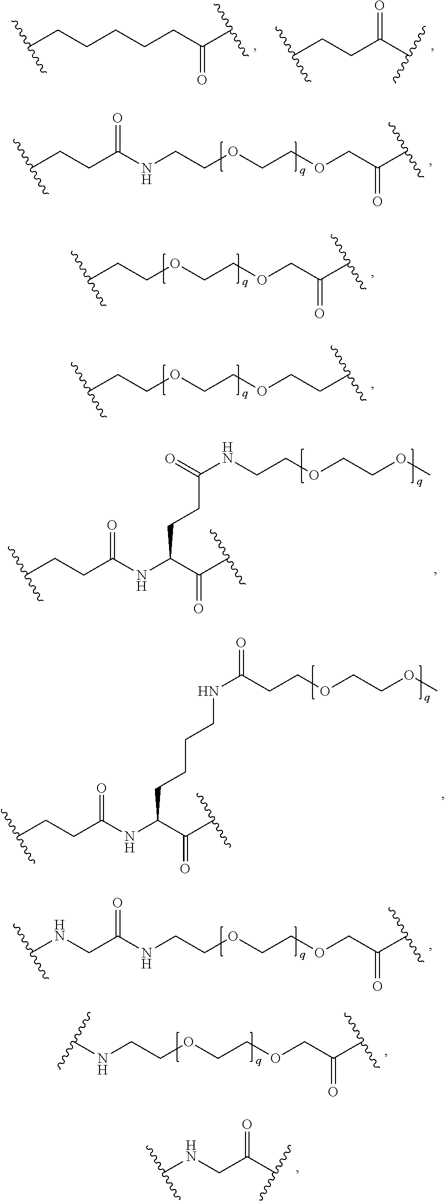
and a single bond, wherein q is an integer from 1 to 20;
L₃ is selected from a single bond or a peptide residue consisting of 1-4 amino acids, wherein the peptide residue is a peptide residue formed by an amino acid selected from the group consisting of:
valine, lysine, cysteine, alanine, glycine, glutamic acid, glutamine, phenylalanine, citrulline, aspartic acid, asparagine, and serine.

27. The ligand-drug conjugate, or the isomer thereof, or the mixture thereof, or the pharmaceutically acceptable salt thereof according to claim 26, wherein L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

28. The ligand-drug conjugate, or the isomer thereof, or the mixture thereof, or the pharmaceutically acceptable salt thereof according to any one of claims 2-27, wherein the linker unit L is -L₃-L₂-L_{1c}-, wherein
L_{1c} is selected from 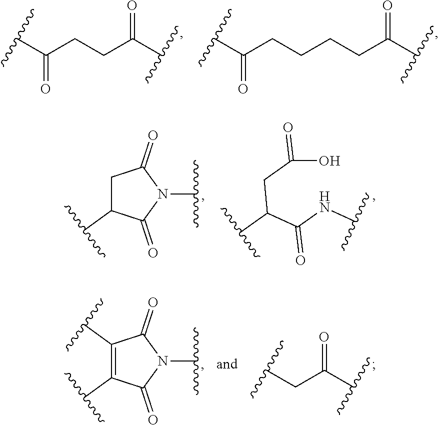 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 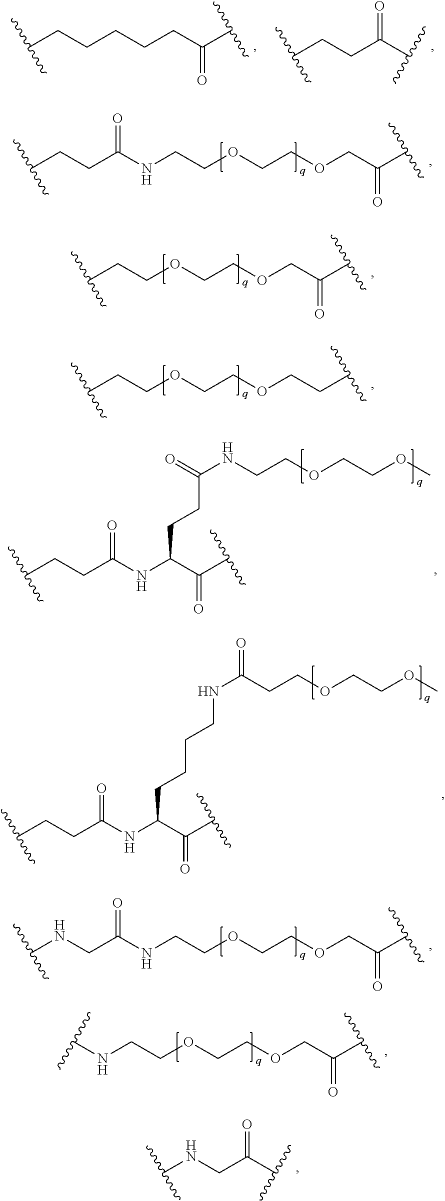
and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

29. The ligand-drug conjugate, or the isomer thereof, or the mixture thereof, or the pharmaceutically acceptable salt thereof according to any one of claims 2-28, wherein the linker unit L is -L₃-L₂-L_{1c}- or -Tr-L₃-L₂-L_{1c}-, wherein the L₃ end or the Tr end is connected to the nitrogen atom of the Z group, and the L_{1c} end is connected to the ligand.

30. The ligand-drug conjugate, or the isomer thereof, or the mixture thereof, or the pharmaceutically acceptable salt thereof according to any one of claims 2-29, wherein the linker unit L is selected from:

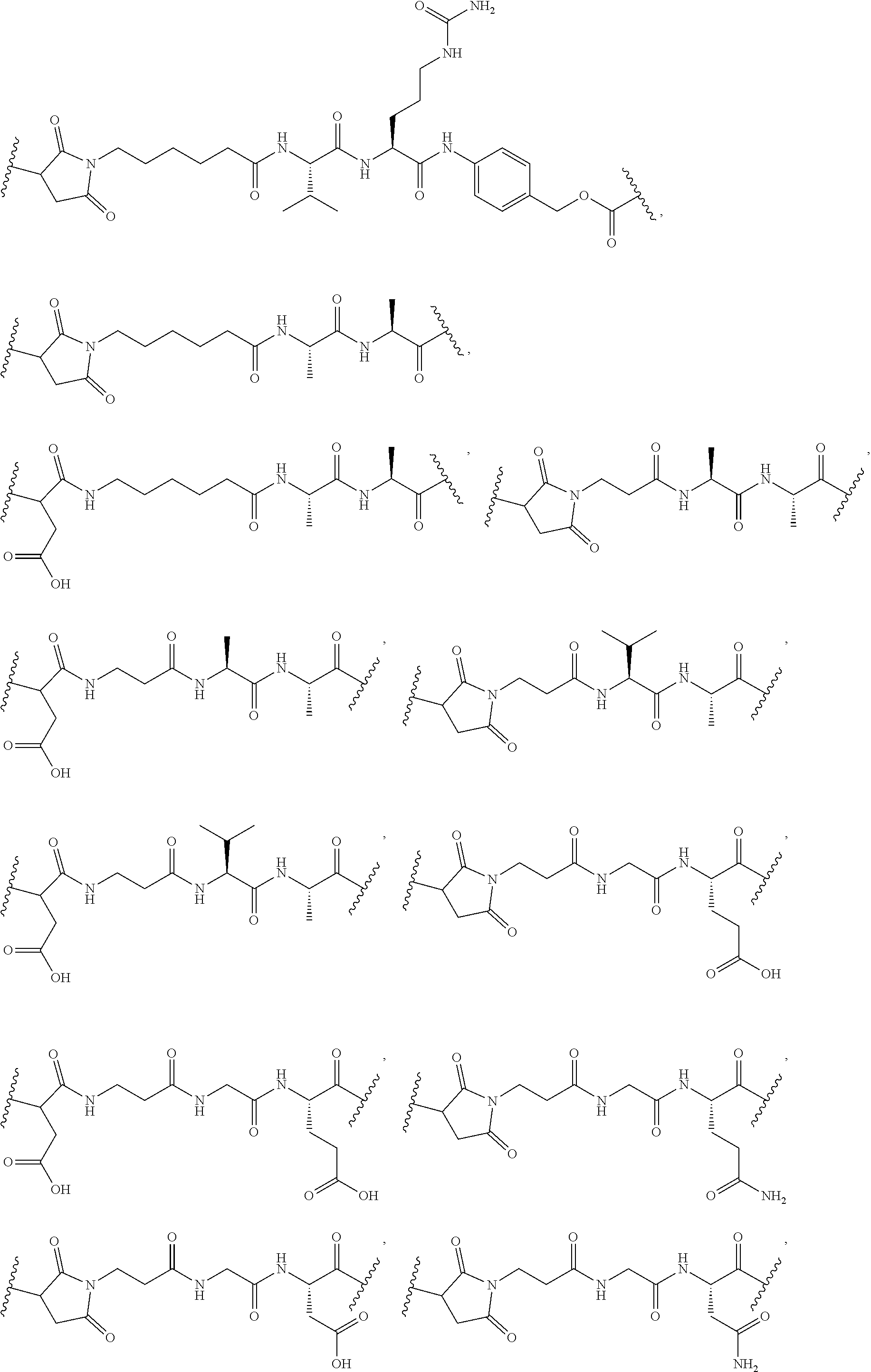

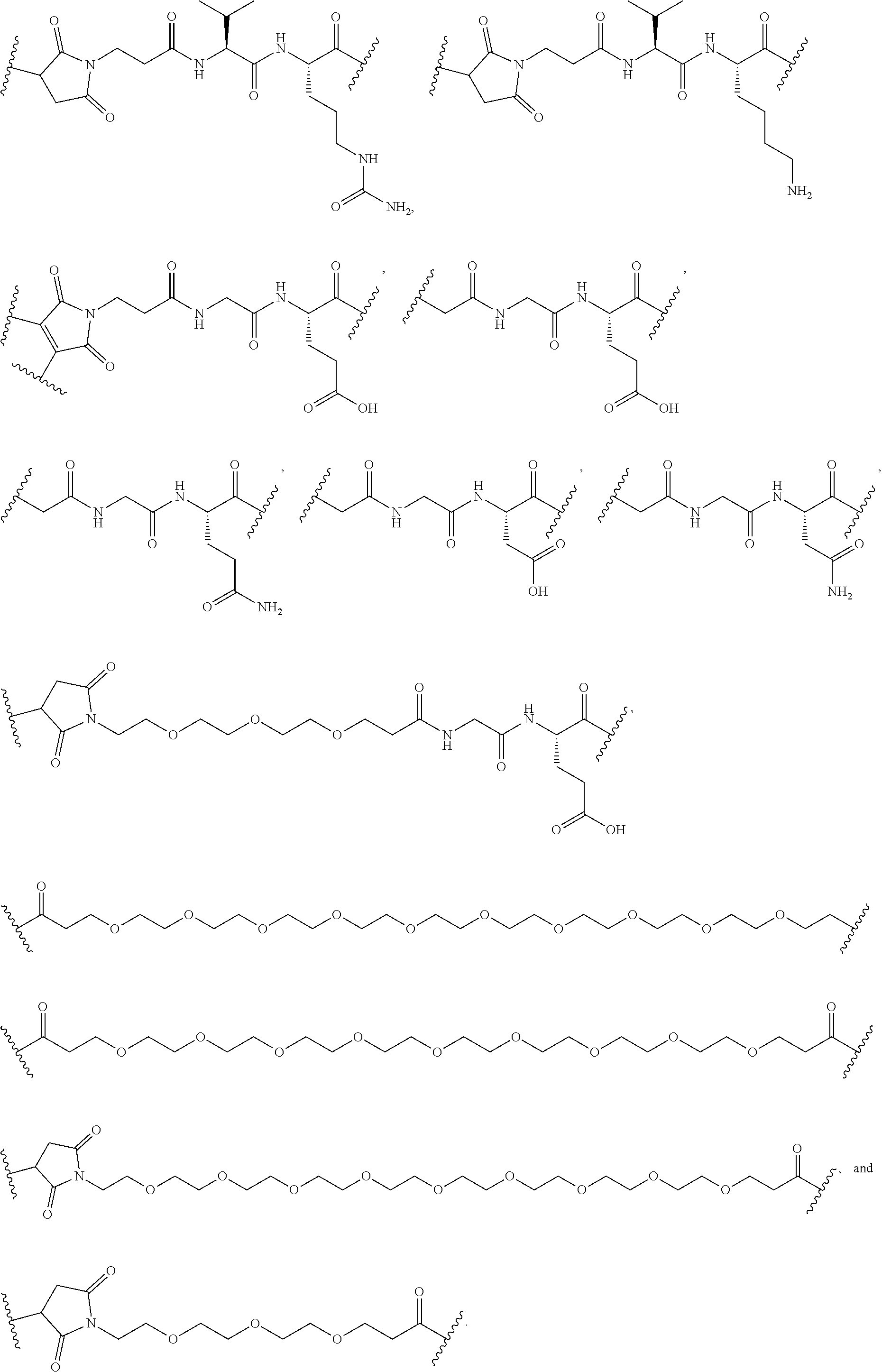

31. The ligand-drug conjugate, or the isomer thereof, or the mixture thereof, or the pharmaceutically acceptable salt thereof according to any one of claims 1-30, wherein the ligand-drug conjugate is represented by formulas (II-2C-1) and (II-2C-2): 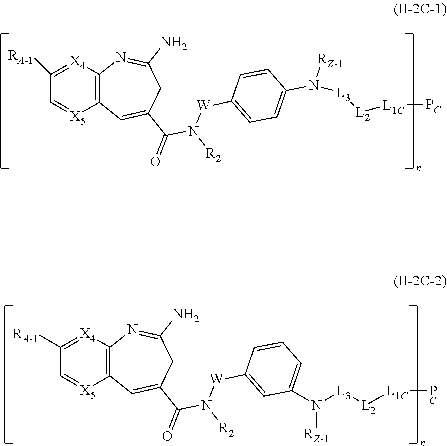
wherein
R_{Z-1} is selected from H or C₁-C₆ alkyl optionally substituted with 1, 2, or 3 fluorine, chlorine, bromine, iodine, hydroxy, or amino substituents;
X₄ and Xₛ are each independently selected from —CH═ and —N═;
R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂₋₂; each R₂₋₂ is independently selected from: hydrogen, halogen, hydroxy, or amino; the methylene units of R₂ are each independently unreplaced, replaced by —O—, or replaced by —N(R₂₋₃)—; R₂₋₃ is selected from: hydrogen or C₁-C₃ alkyl;
W is selected from C₁-C₆ alkyl optionally substituted with 1, 2, or 3 R_{w-1}; each R_{w-1} is independently selected from hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2}, each R_{w-2} is independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl;
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein
each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆ alkyl), or —N(C₁-C₆ alkyl)₂;
L_{1c} is selected from 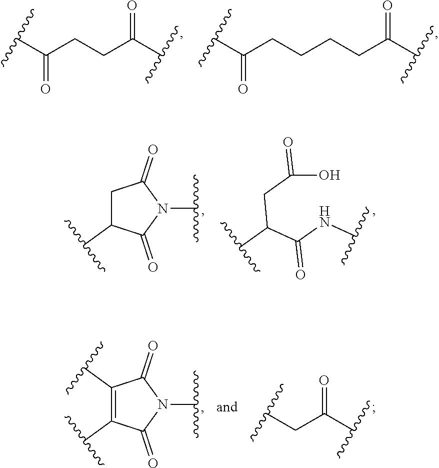 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 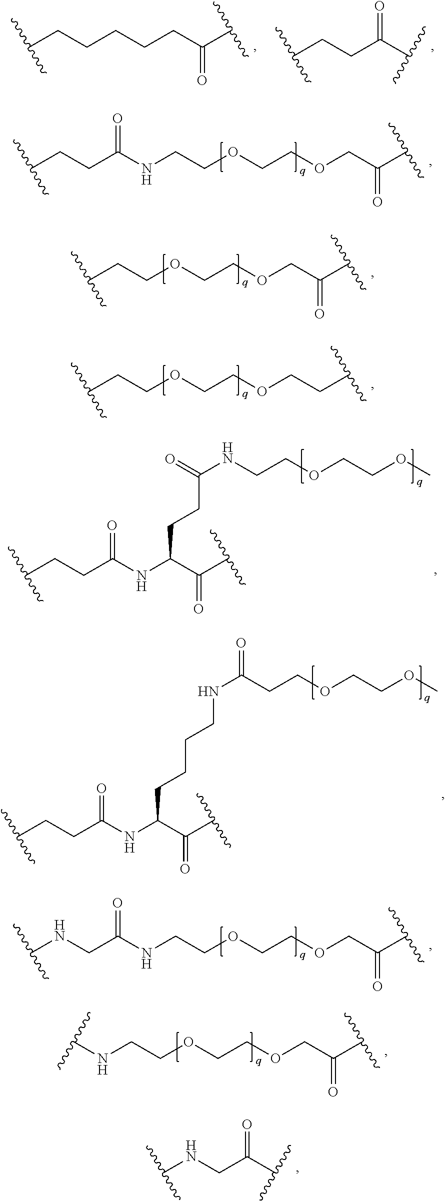
and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-;
n is an integer or a decimal from 1 to 10;
Pc is a ligand.

32. The ligand-drug conjugate, or the isomer thereof, or the mixture thereof, or the pharmaceutically acceptable salt thereof according to any one of claims 1-31, wherein the ligand-drug conjugate is selected from the following structural formulas: 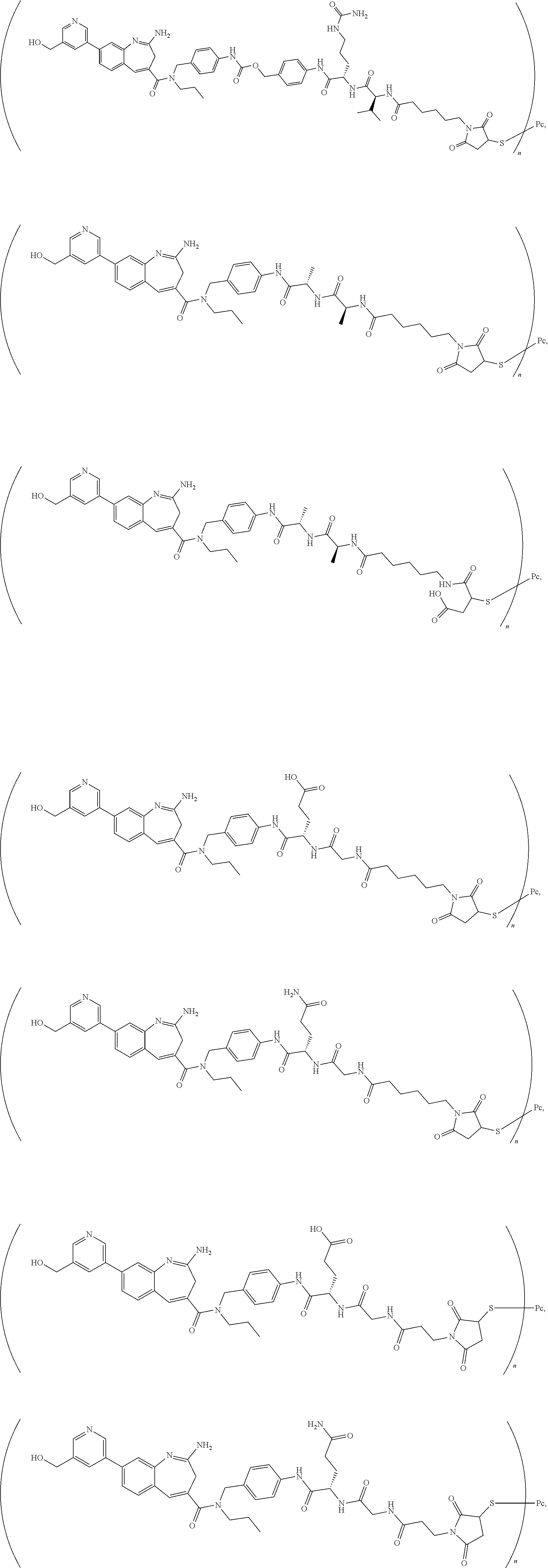 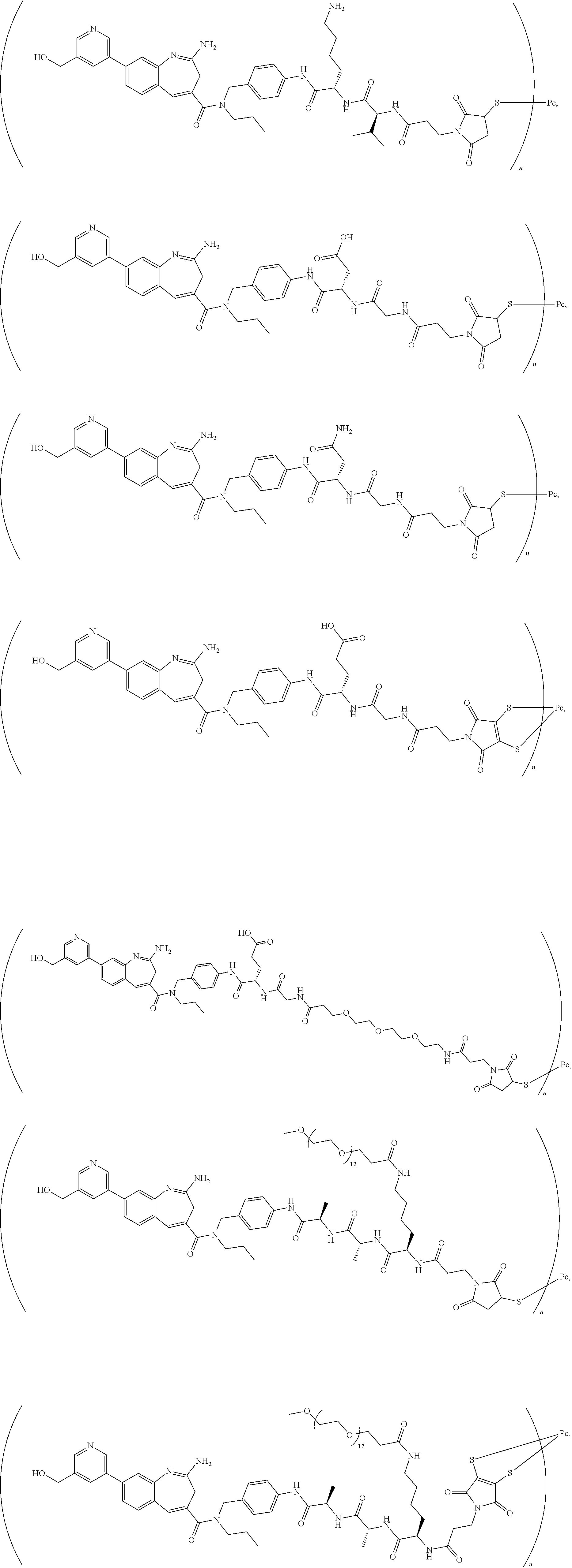 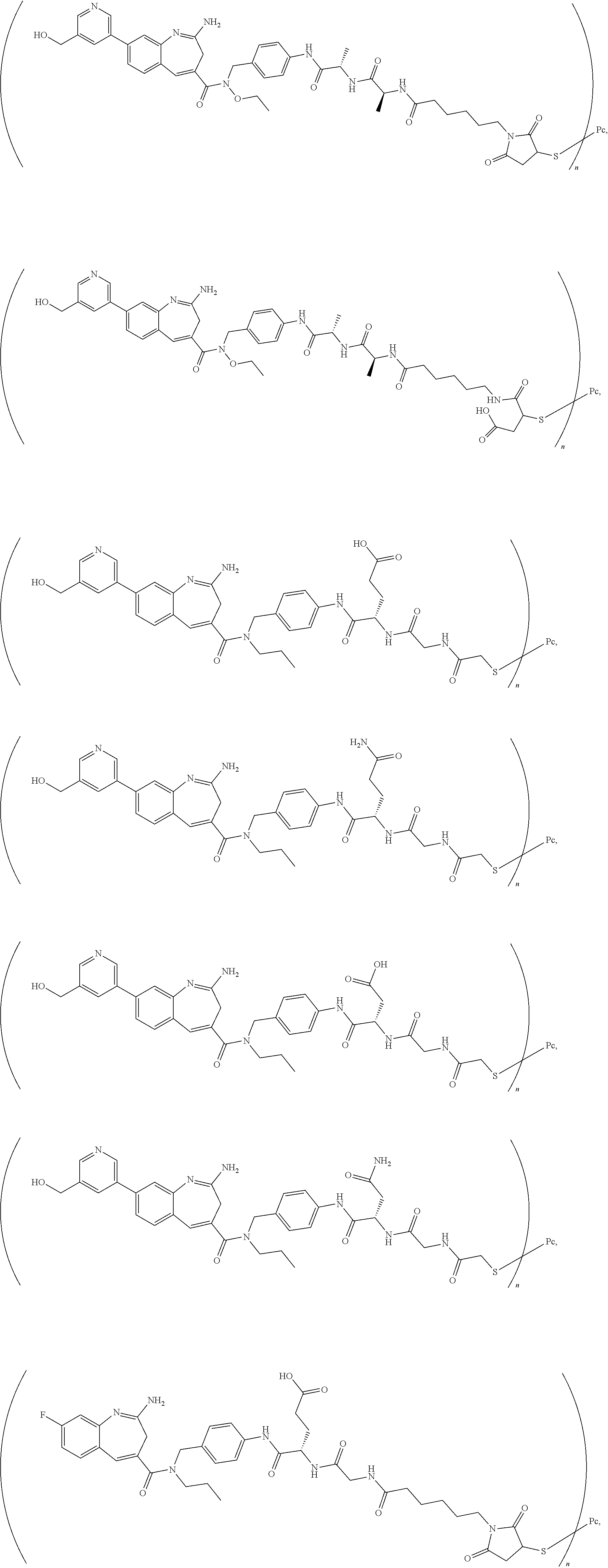 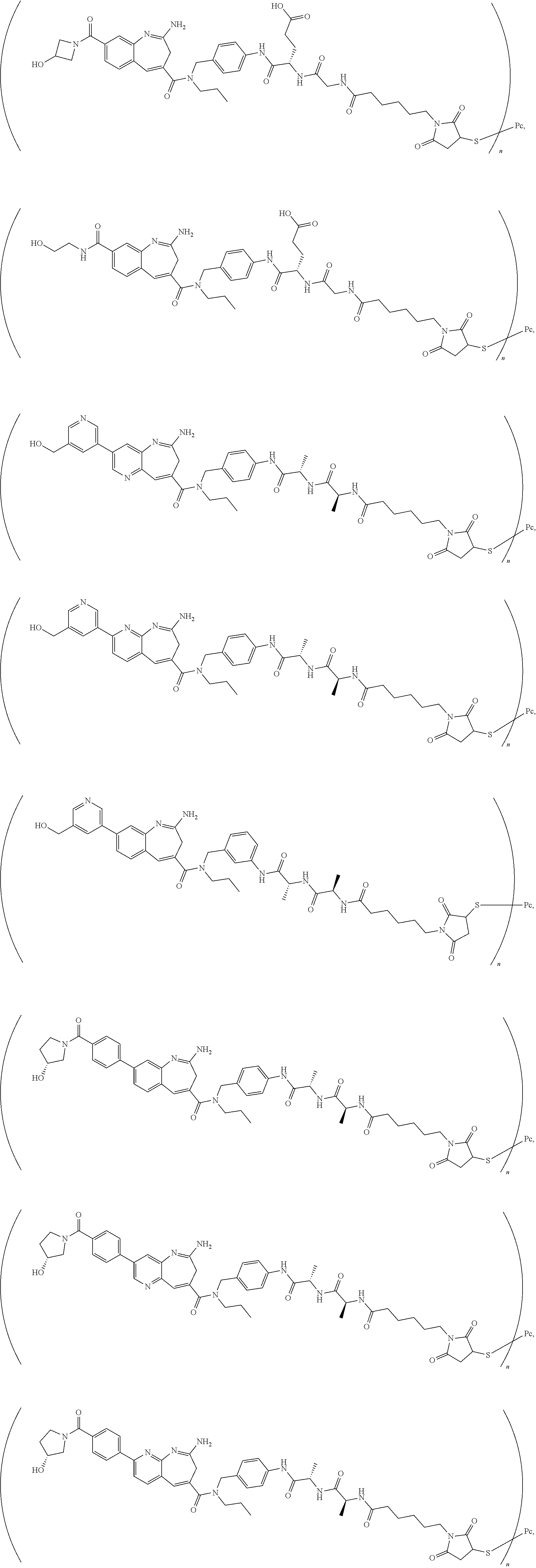 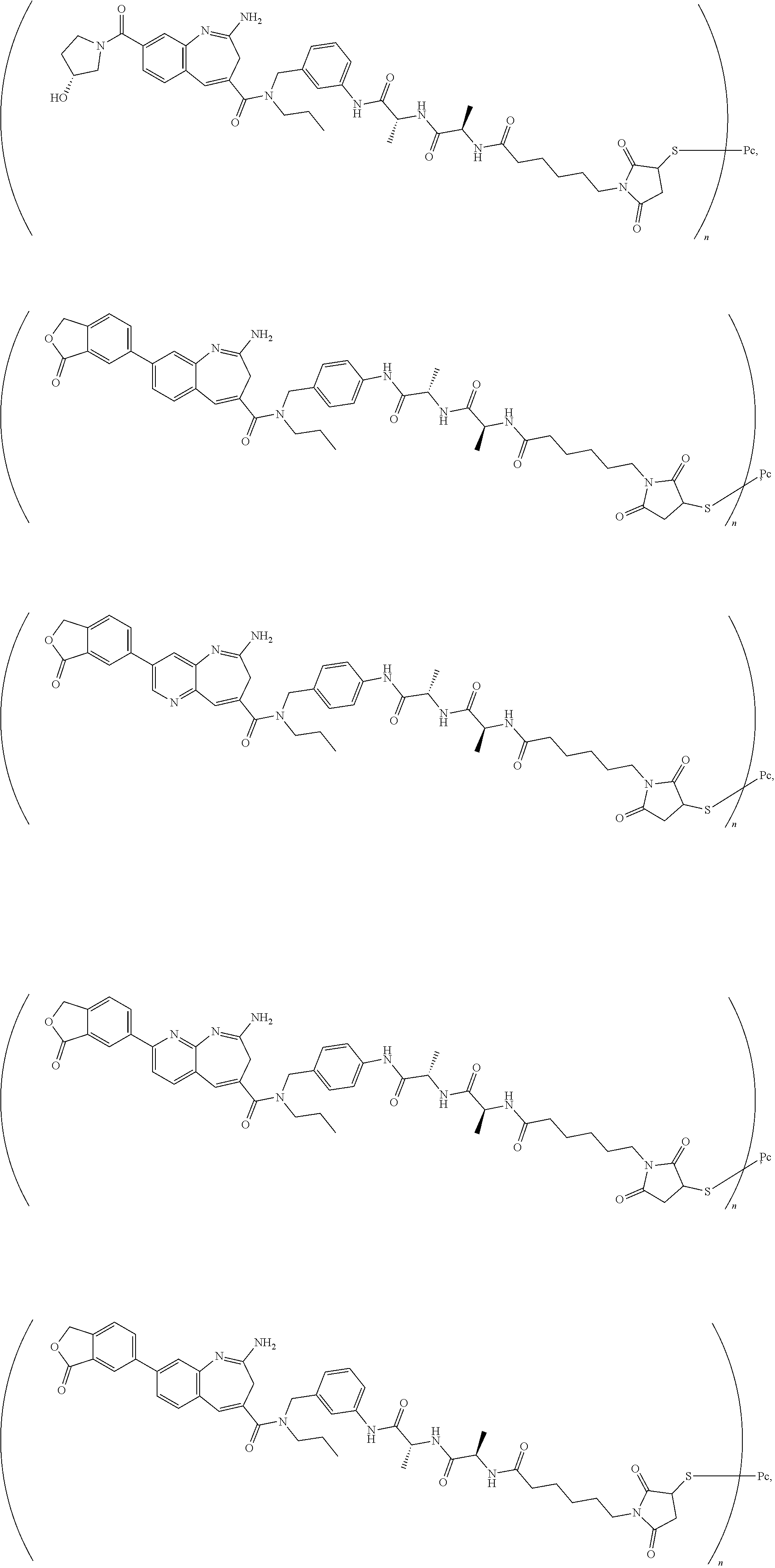 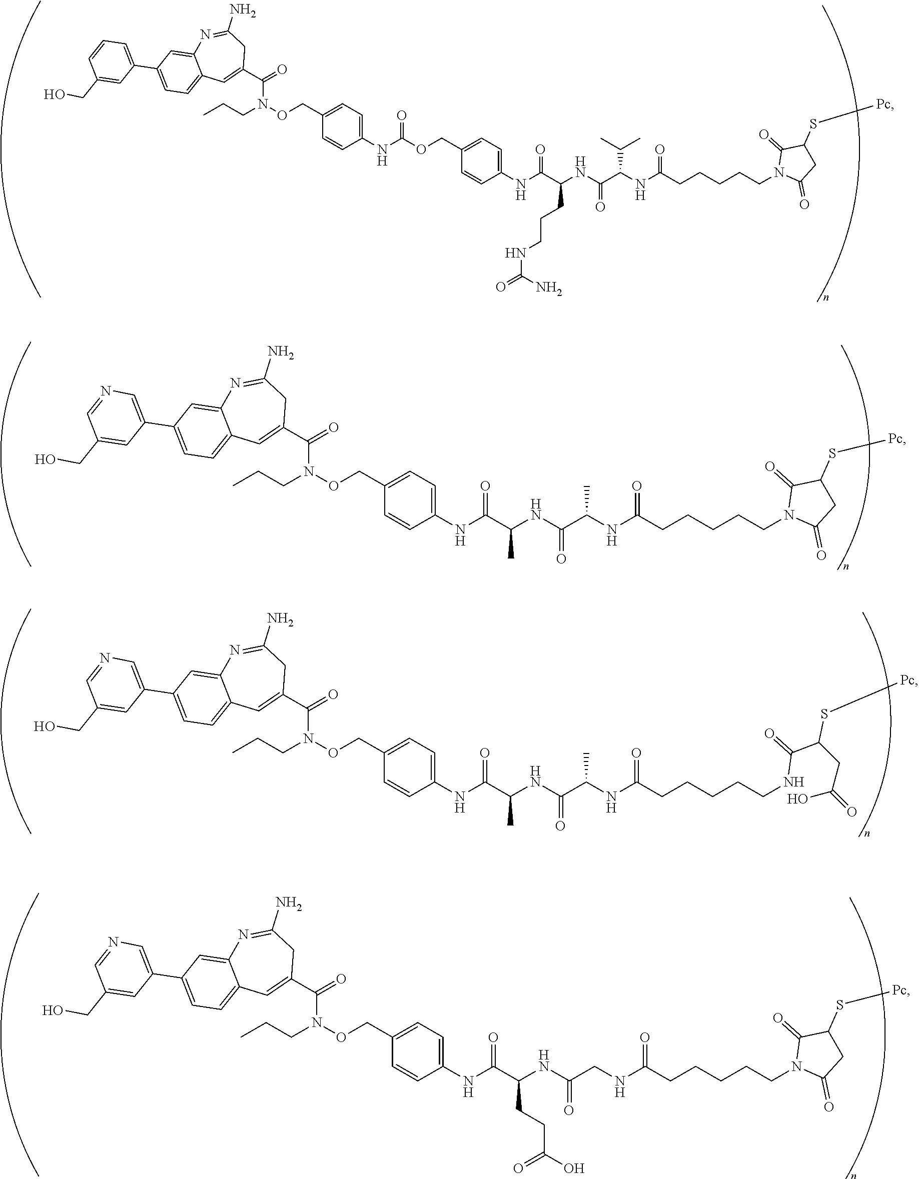 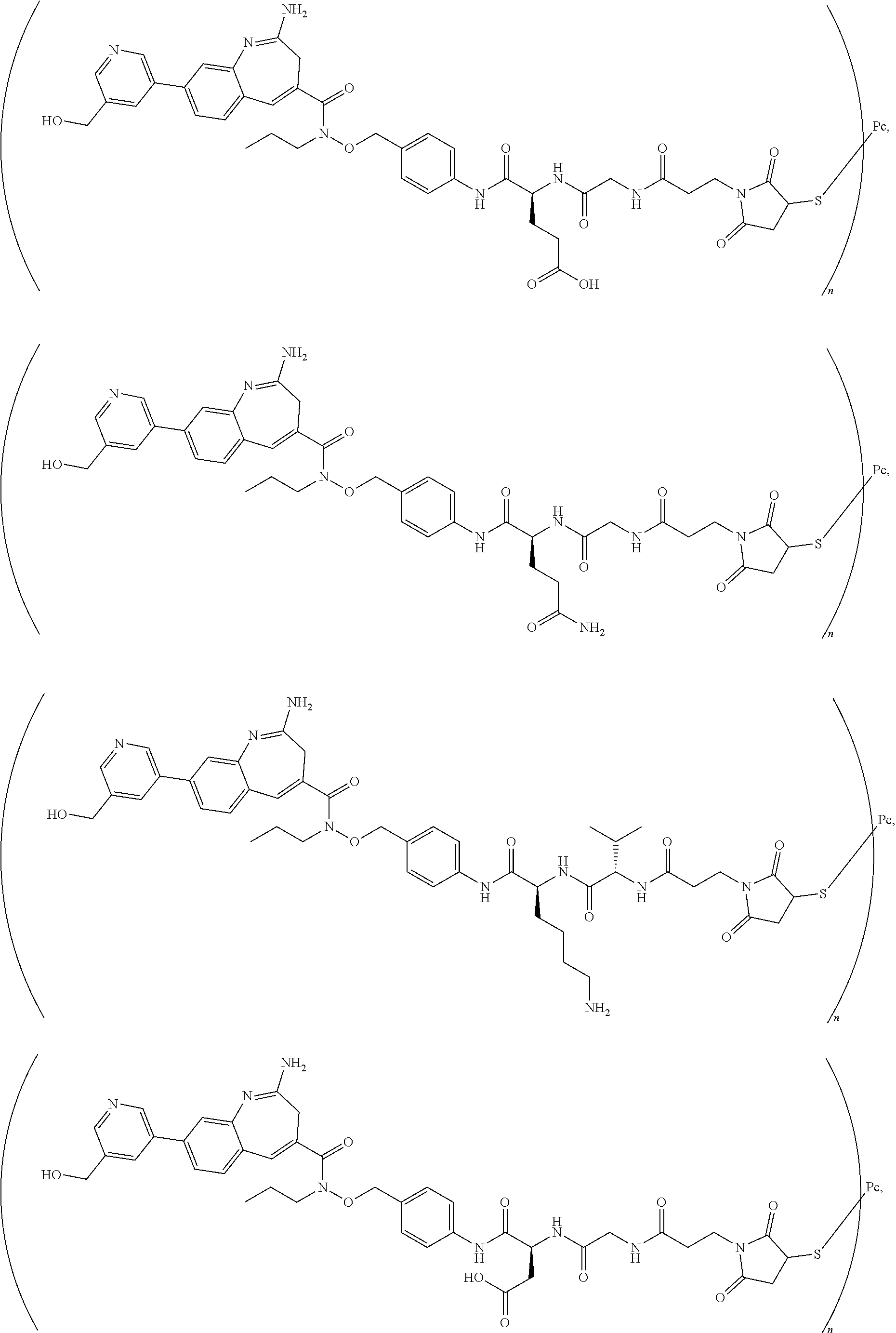 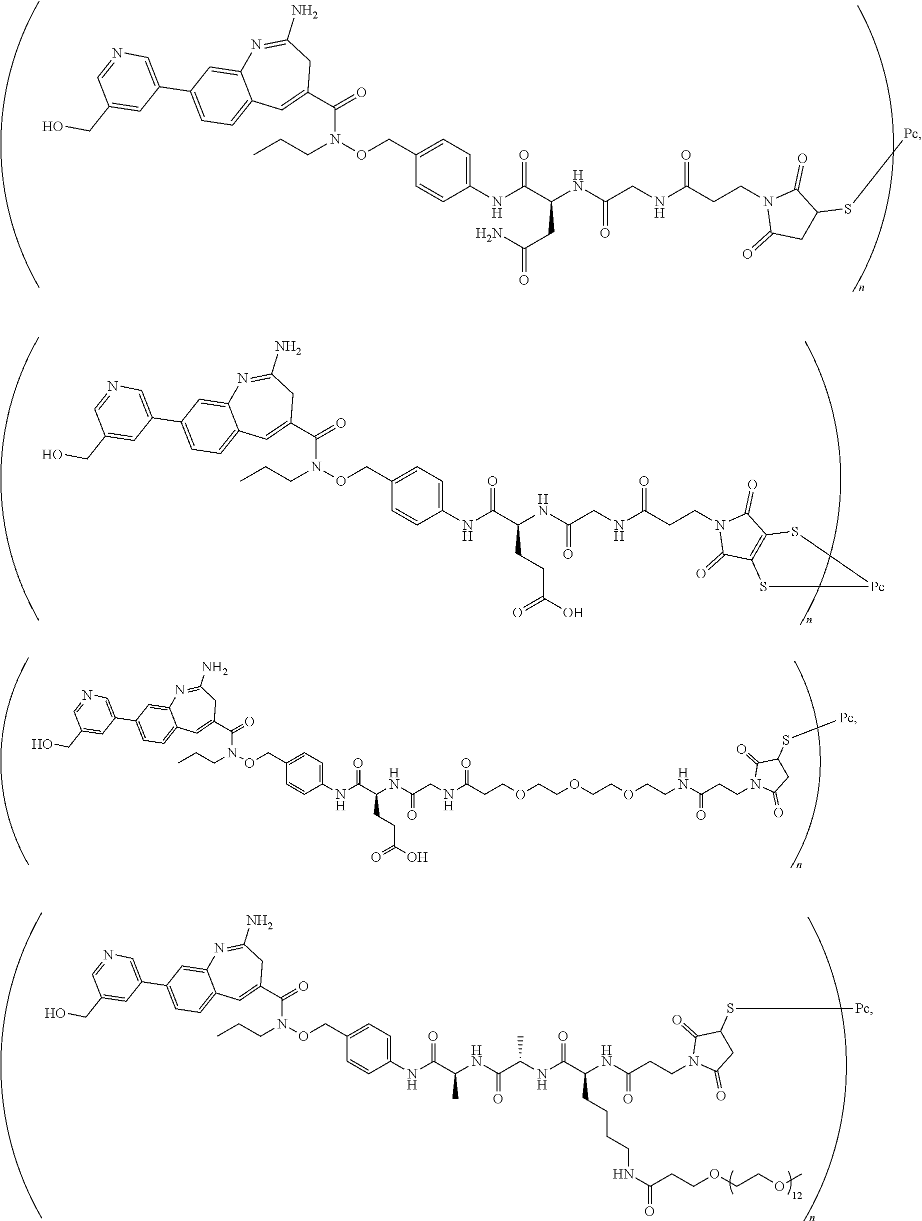 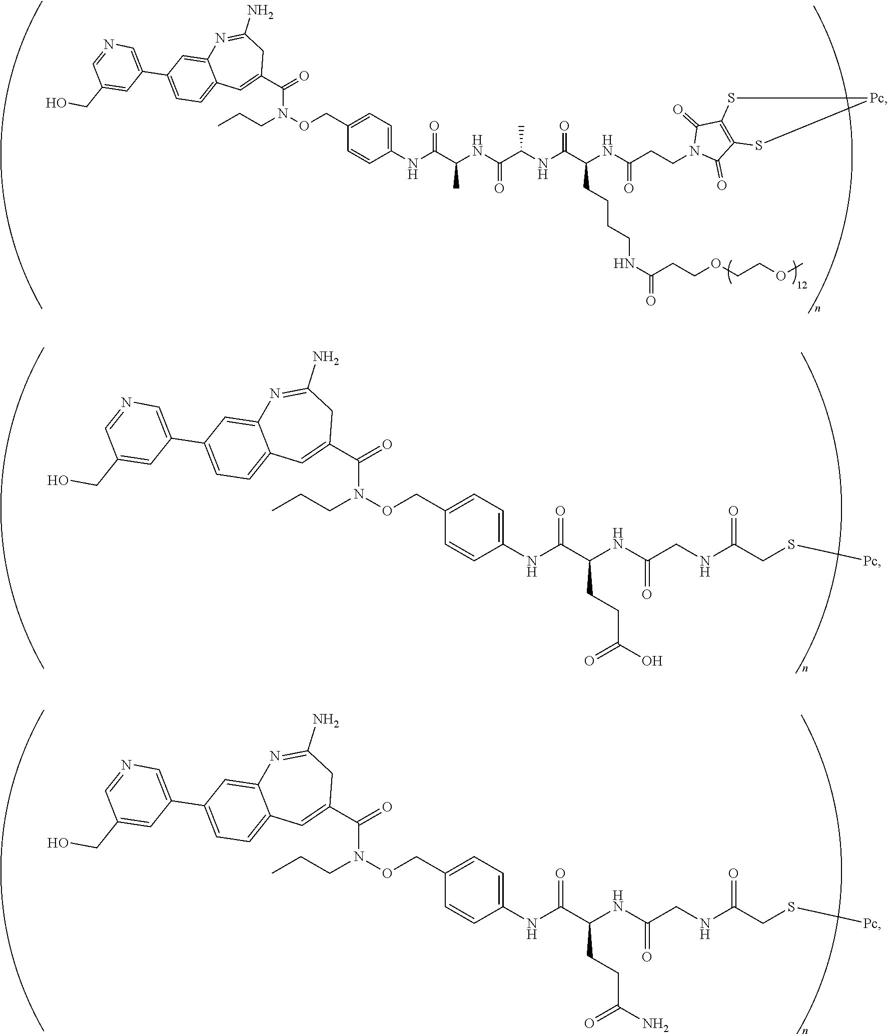 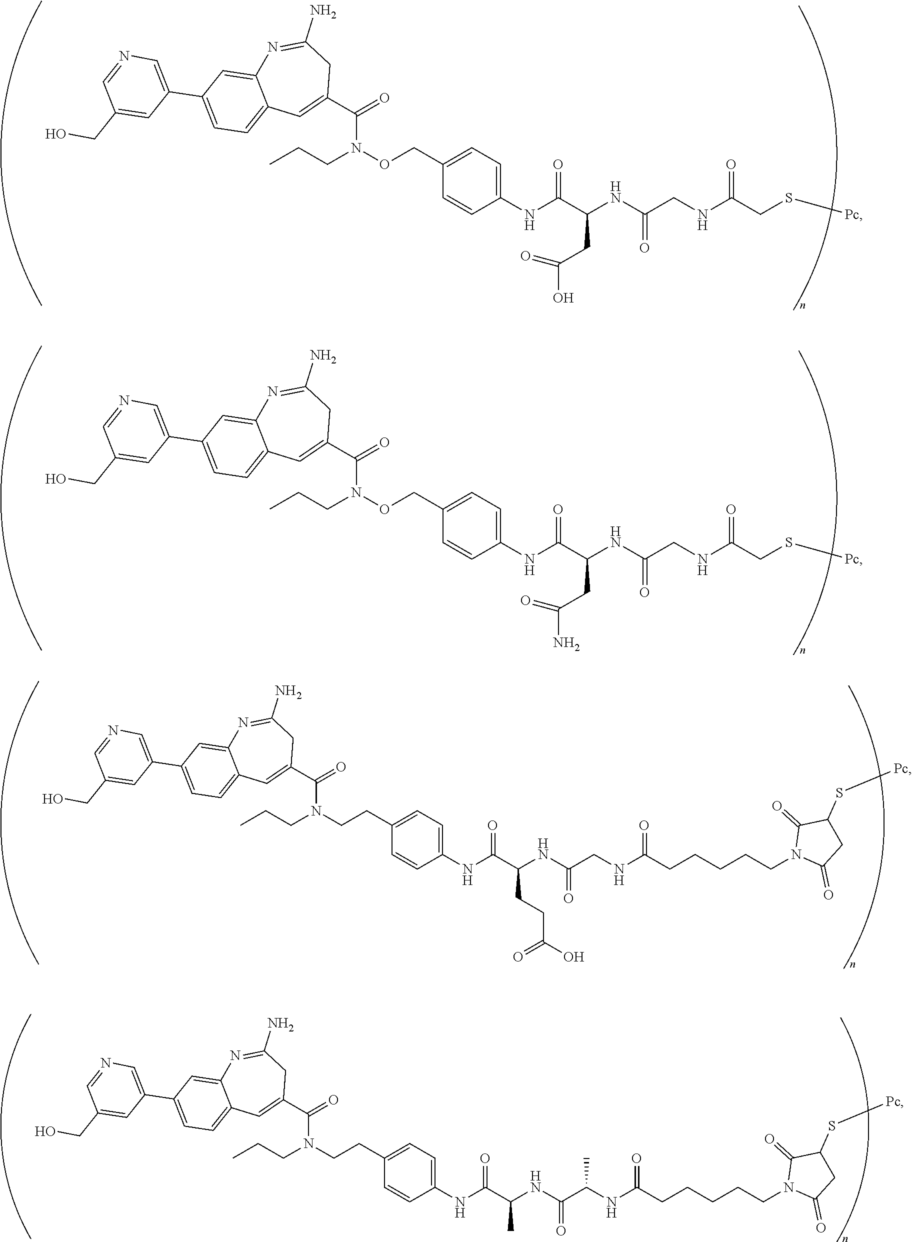 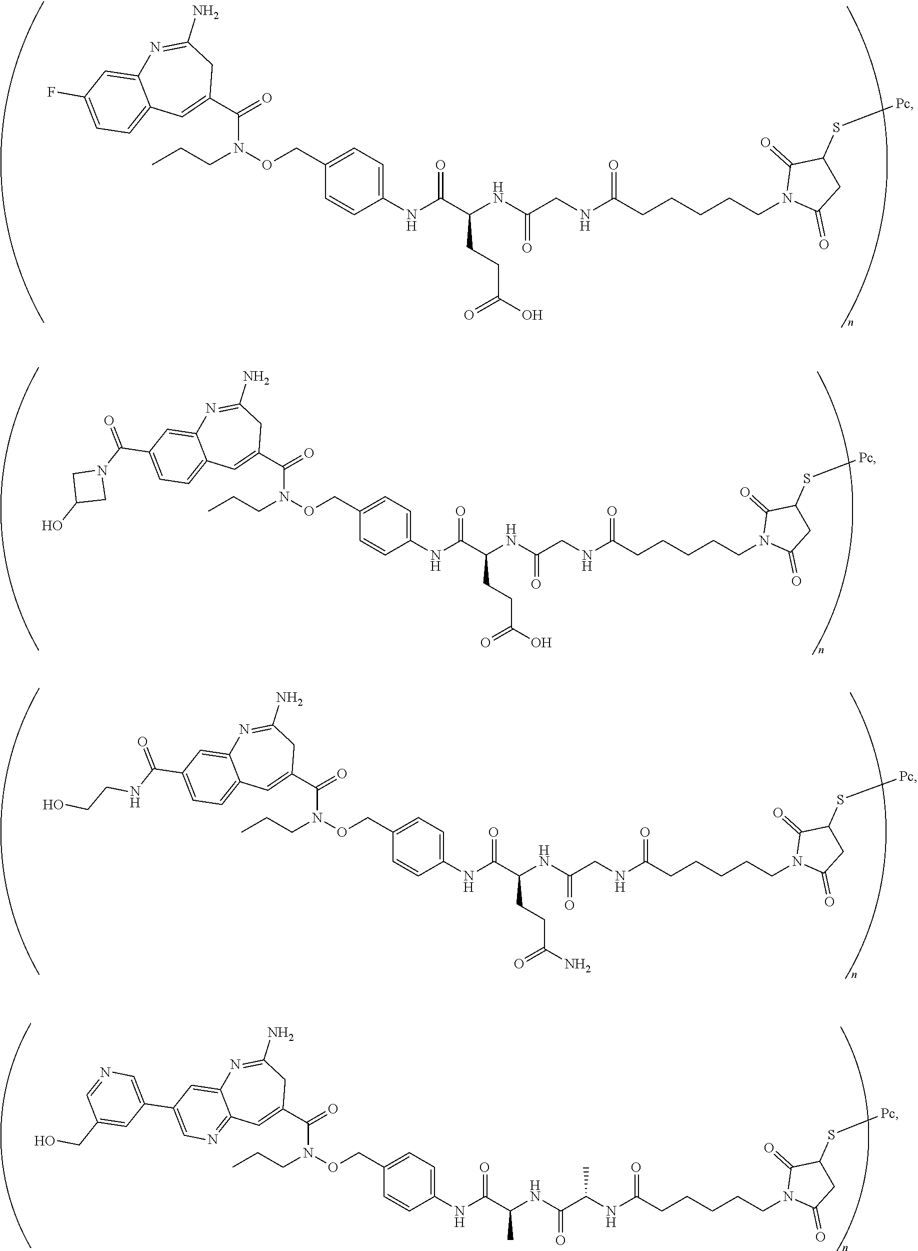 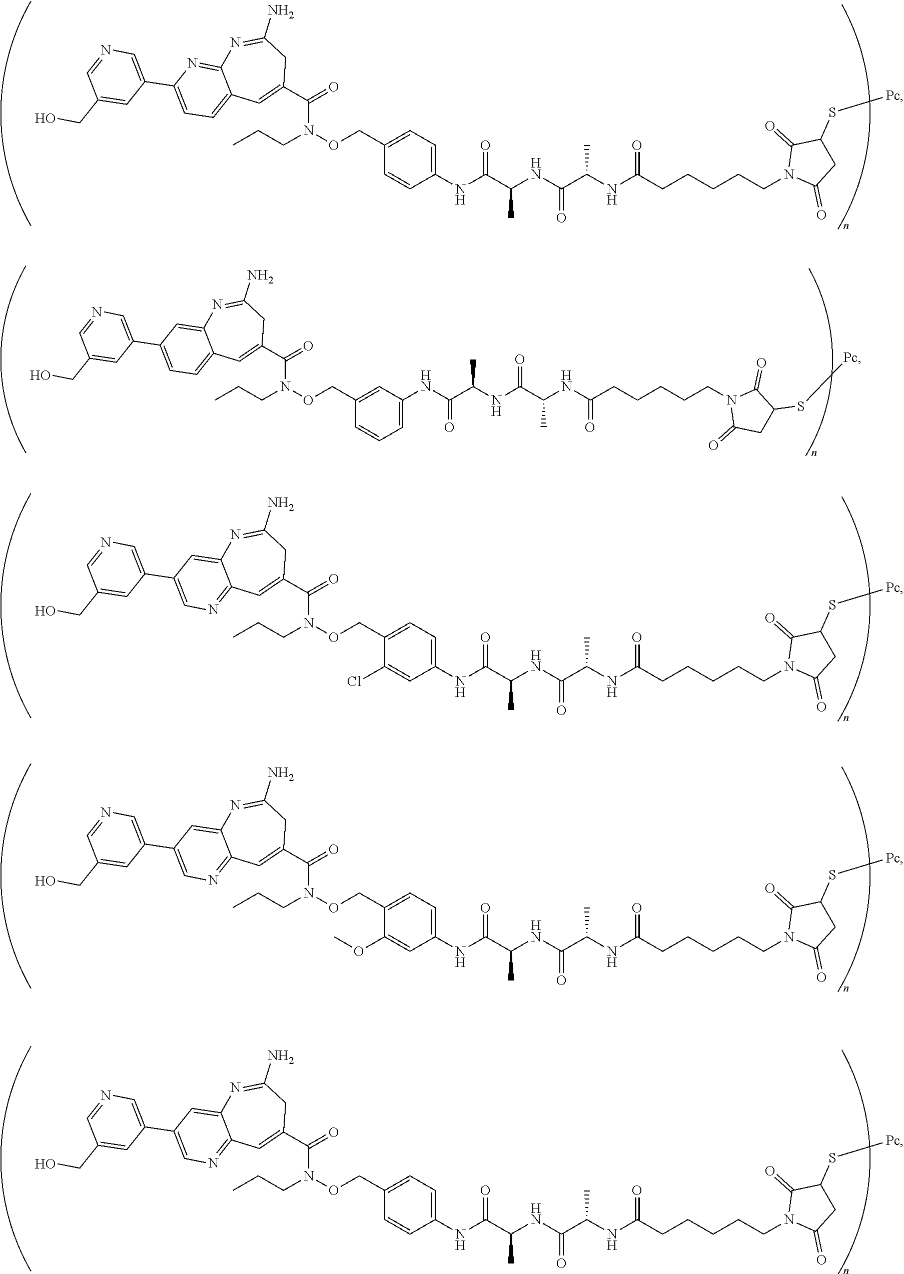 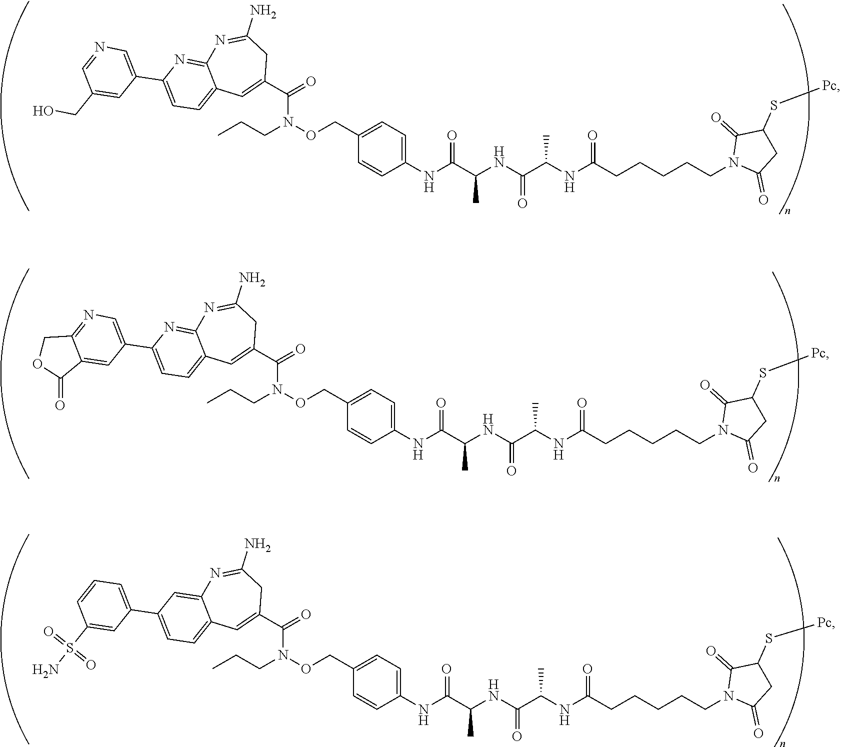 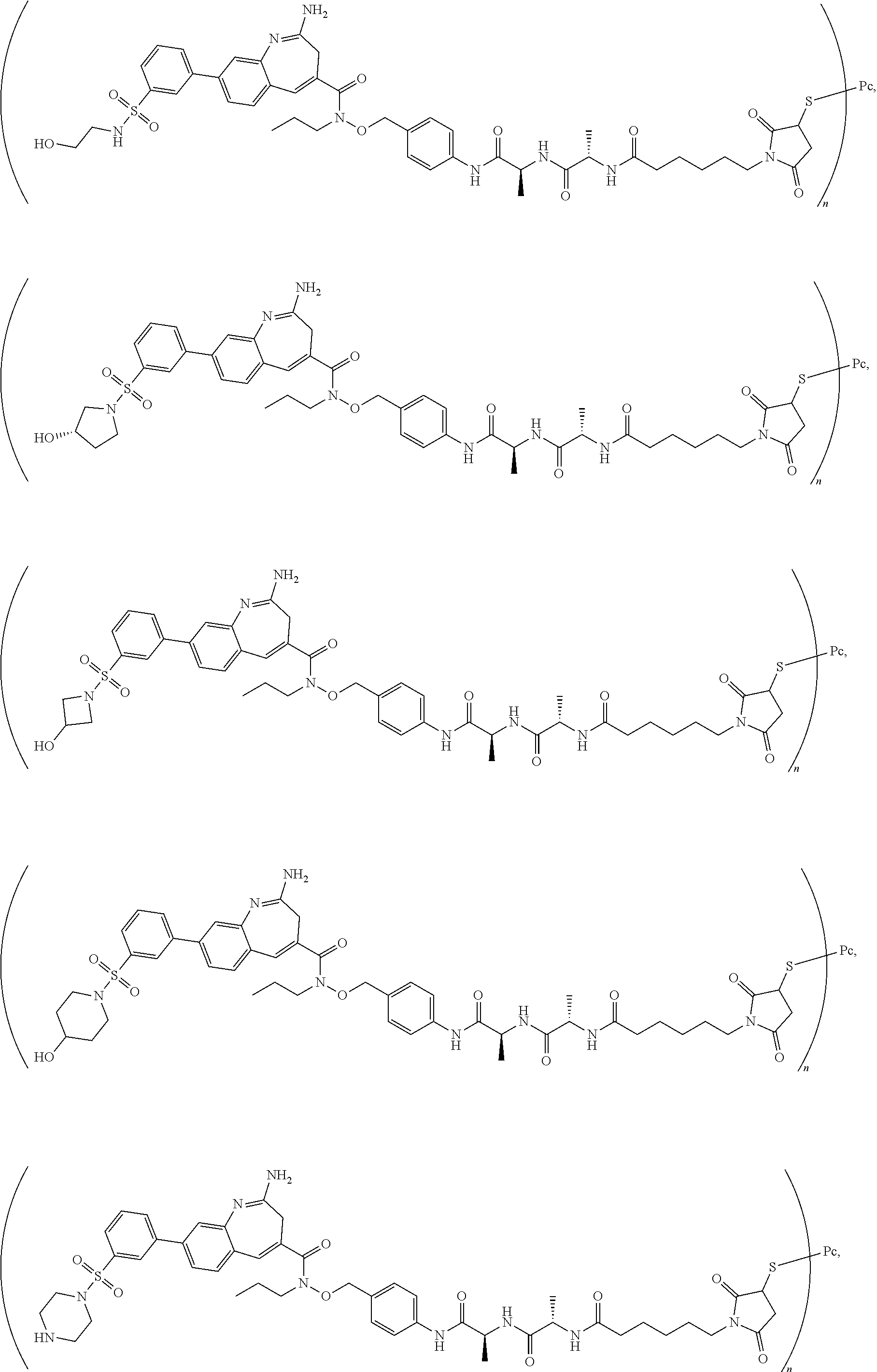 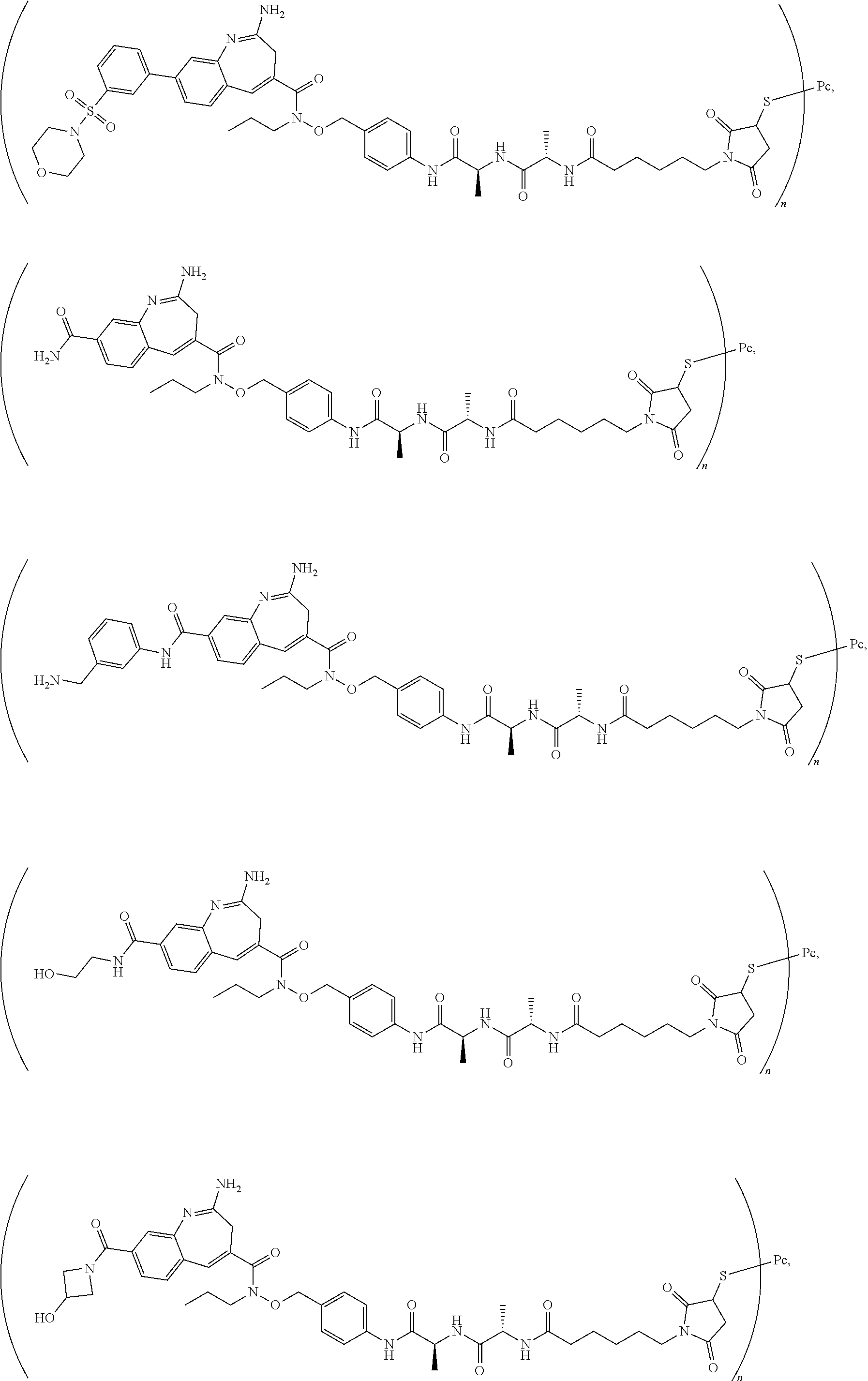 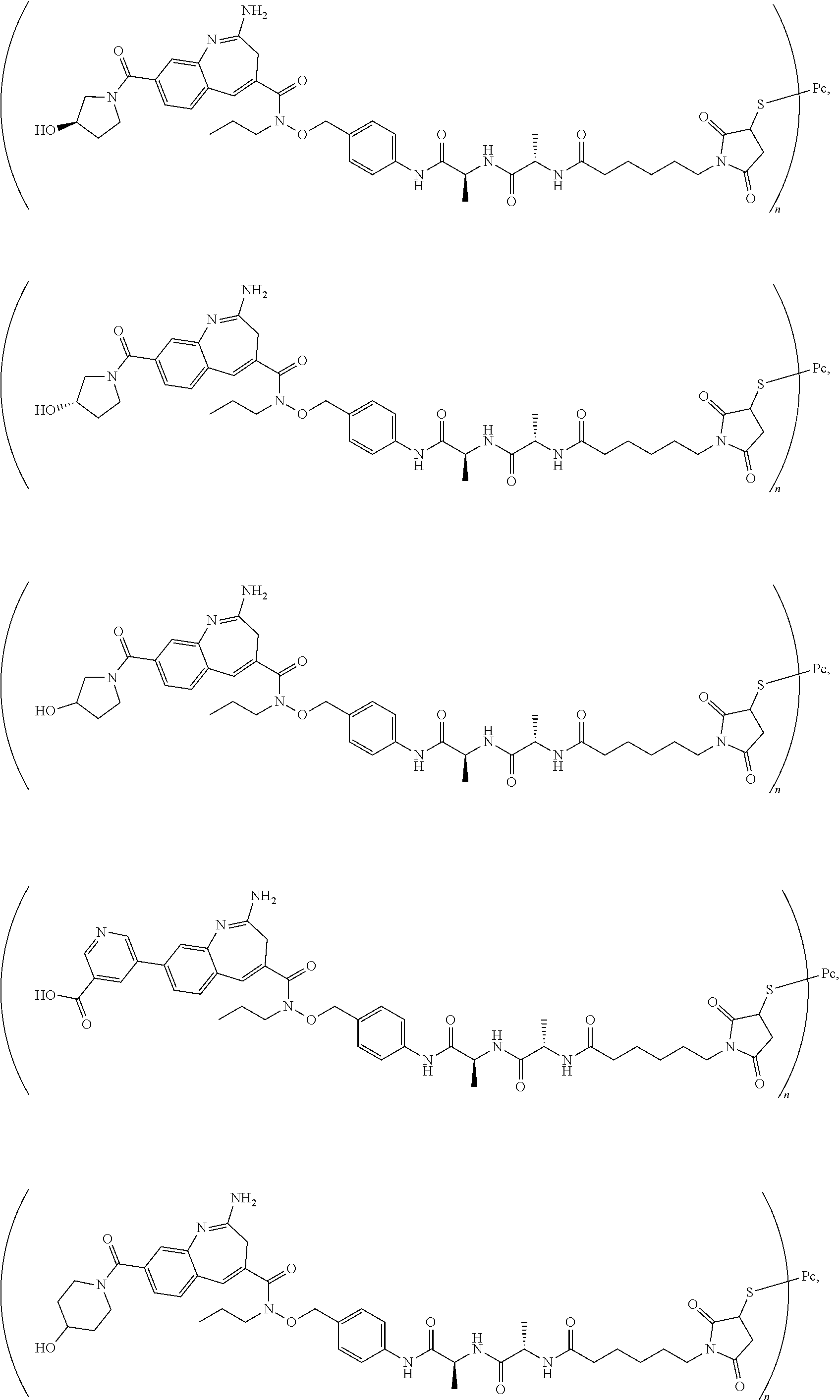 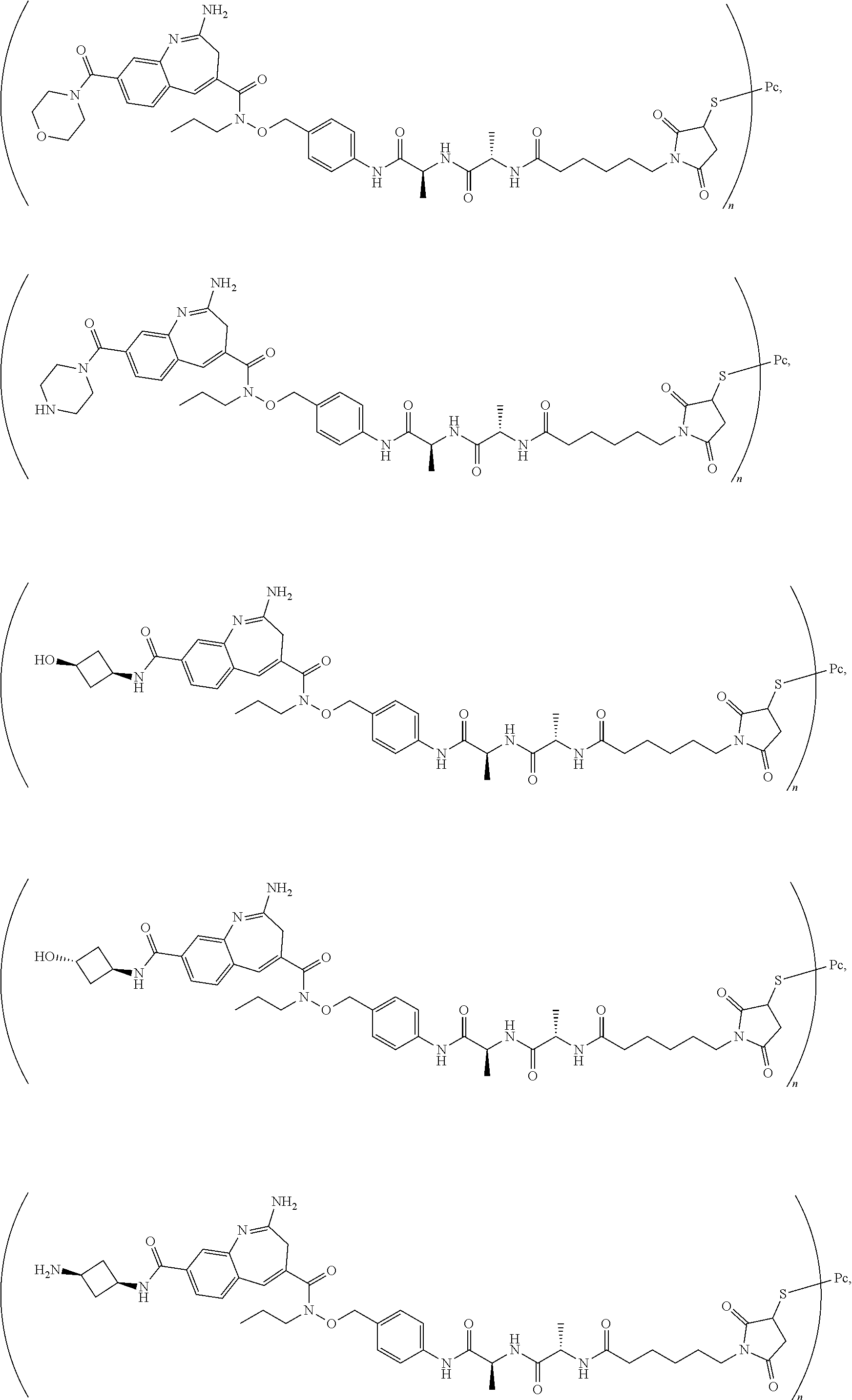 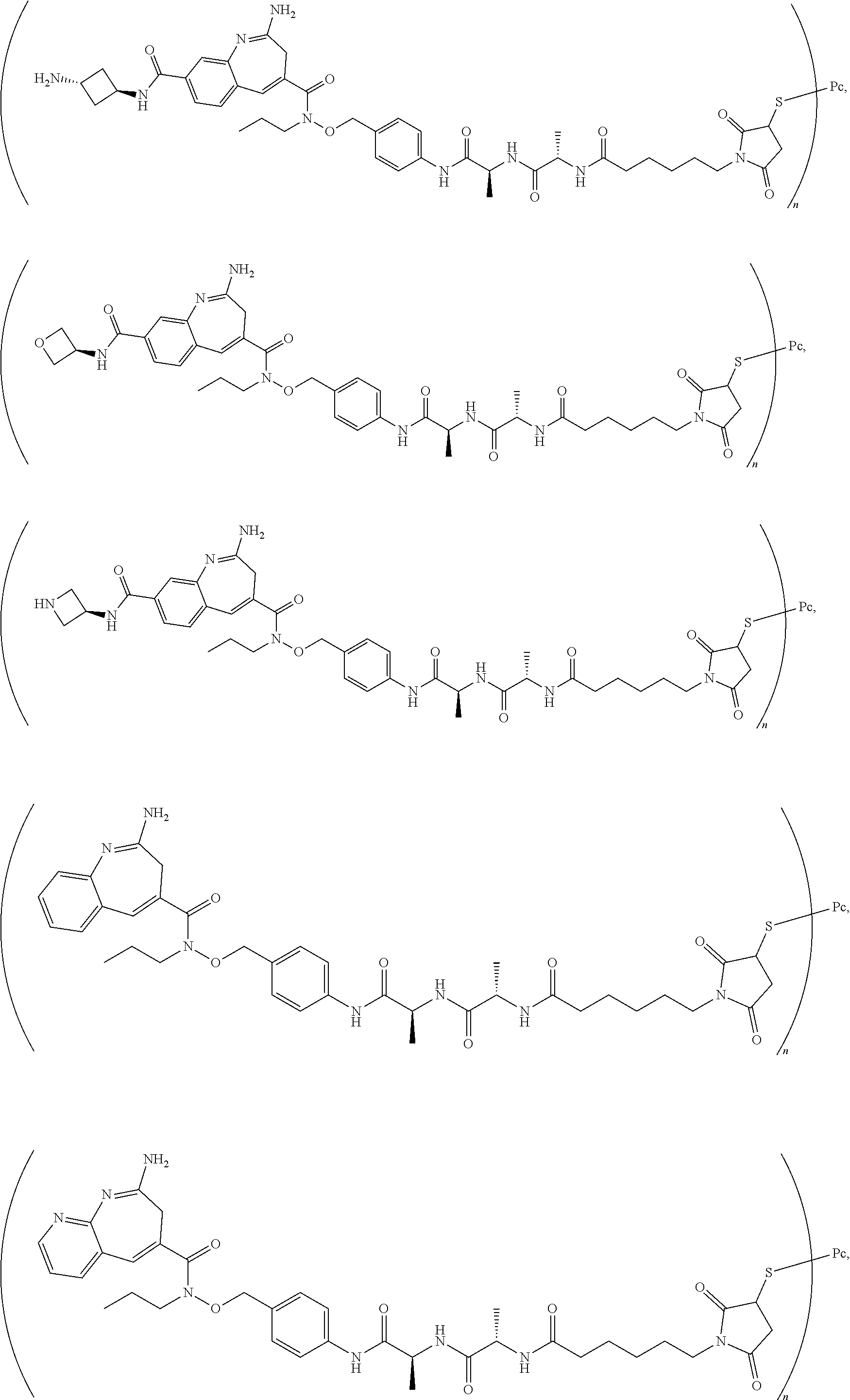 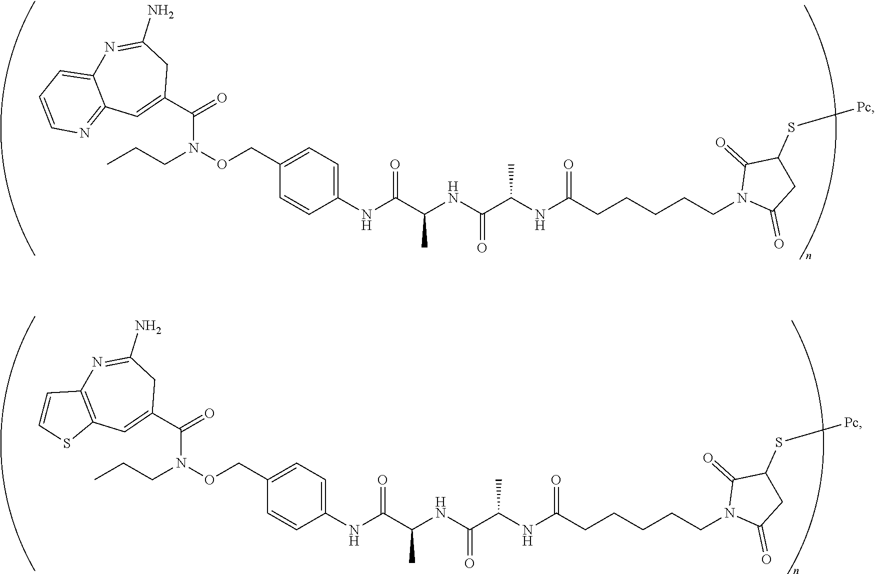 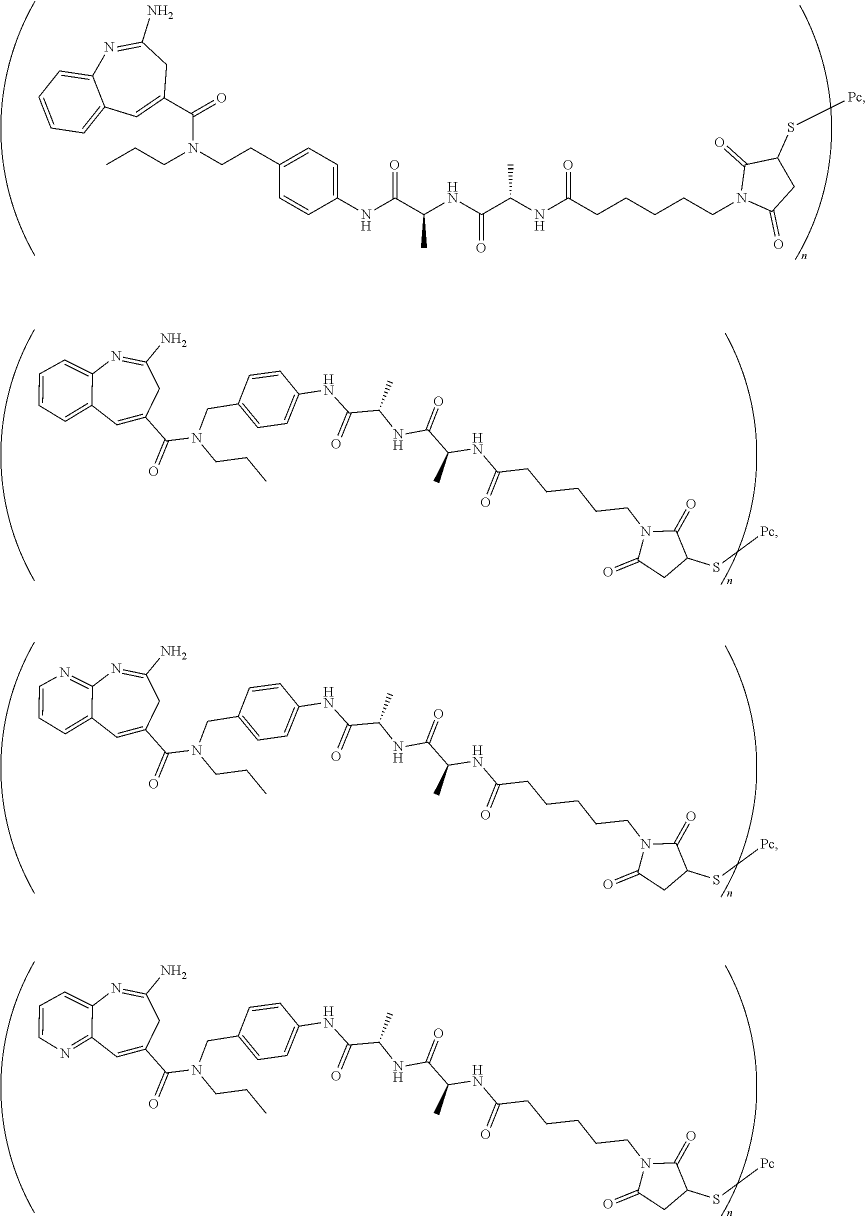
wherein
n is an integer or a decimal from 1 to 10;
Pc is a ligand.

33. The ligand-drug conjugate, or the isomer thereof, or the mixture thereof, or the pharmaceutically acceptable salt thereof according to any one of claims 3-32, wherein n is an integer or a decimal from 2 to 8.

34. The ligand-drug conjugate, or the isomer thereof, or the mixture thereof, or the pharmaceutically acceptable salt thereof according to any one of claims 3-33, wherein Pc is an antibody or an antigen-binding fragment thereof.

35. The ligand-drug conjugate, or the isomer thereof, or the mixture thereof, or the pharmaceutically acceptable salt thereof according to claims 3-34, wherein Pc targets GPC3, Trop2, and HER2; preferably, the antibody is selected from: trastuzumab, pertuzumab, sacituzumab, and codrituzumab.

36. The ligand-drug conjugate, or the isomer thereof, or the mixture thereof, or the pharmaceutically acceptable salt thereof according to any one of claims 1-35, wherein the ligand-drug conjugate is selected from the following structural formulas: 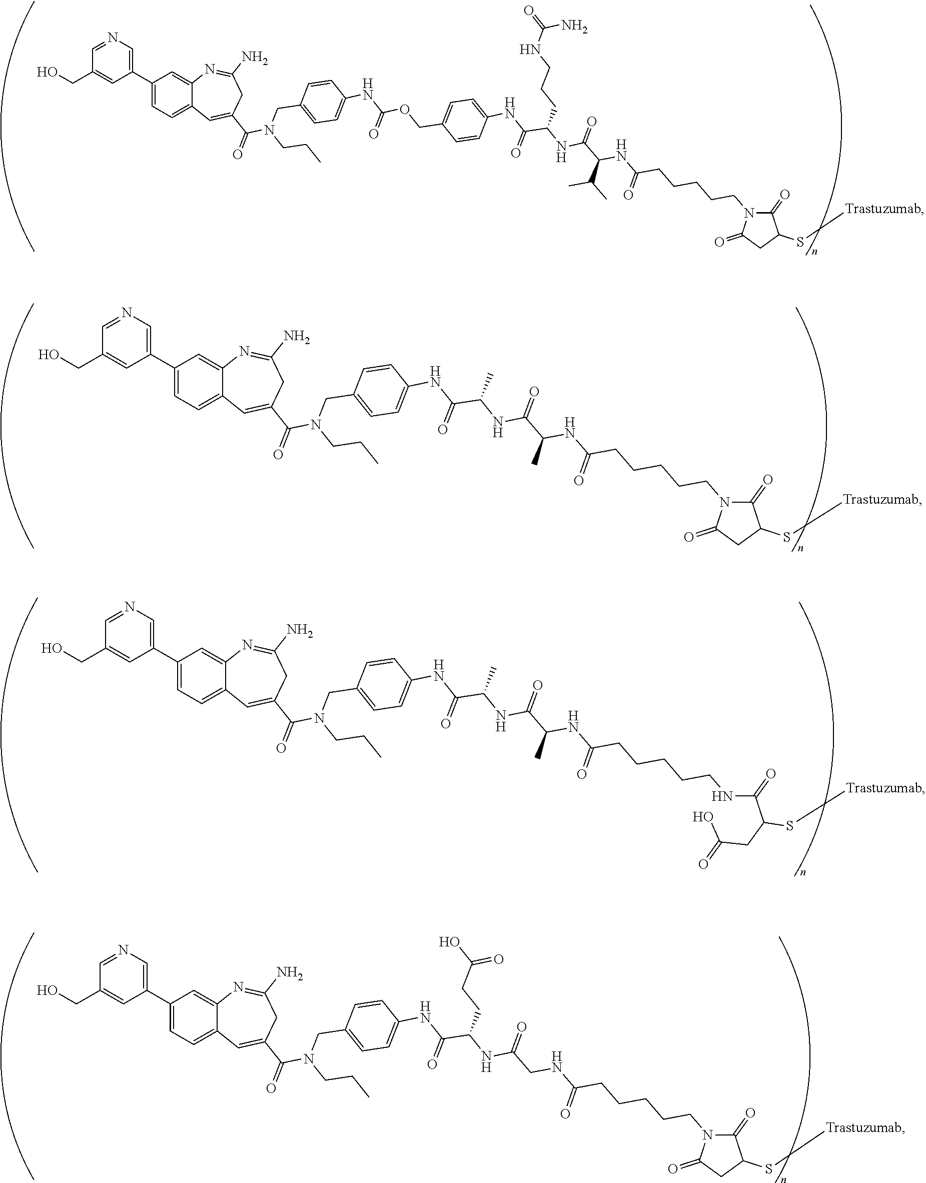 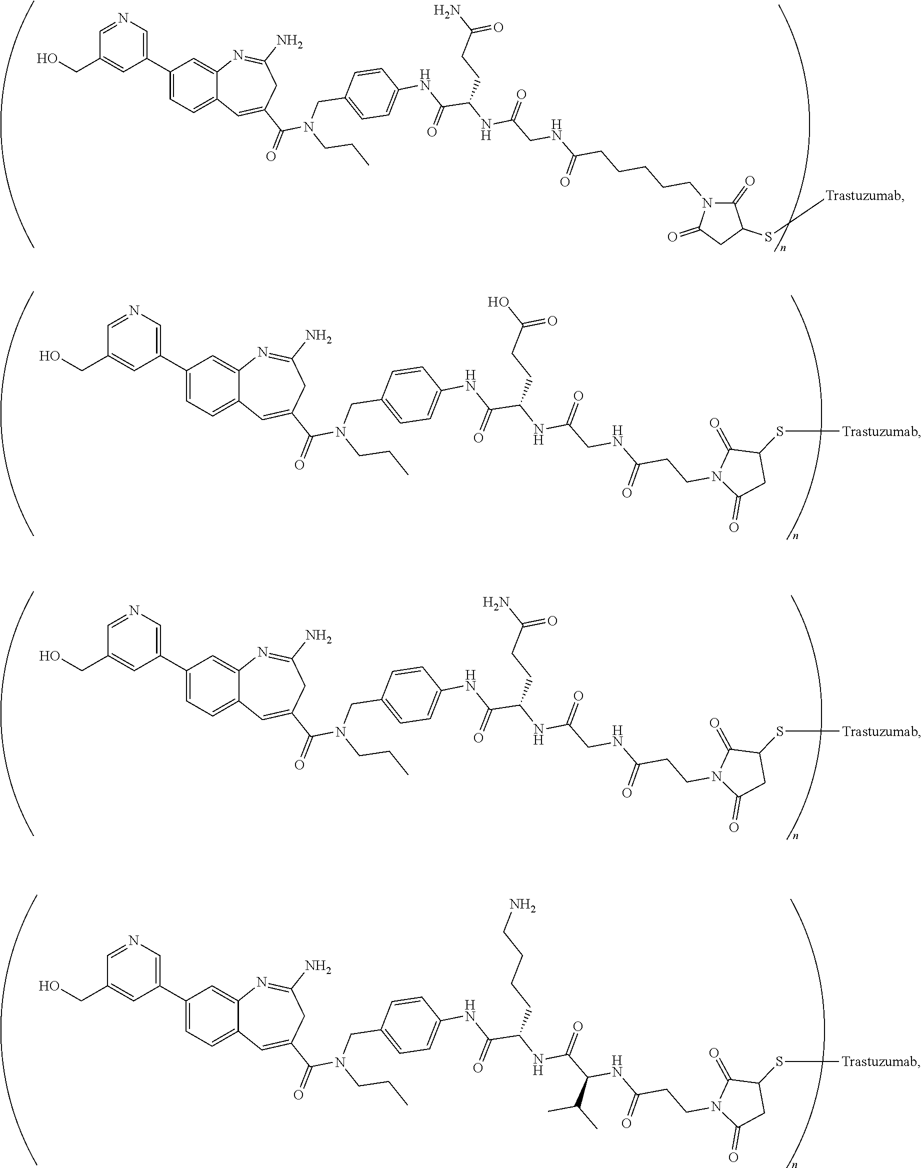 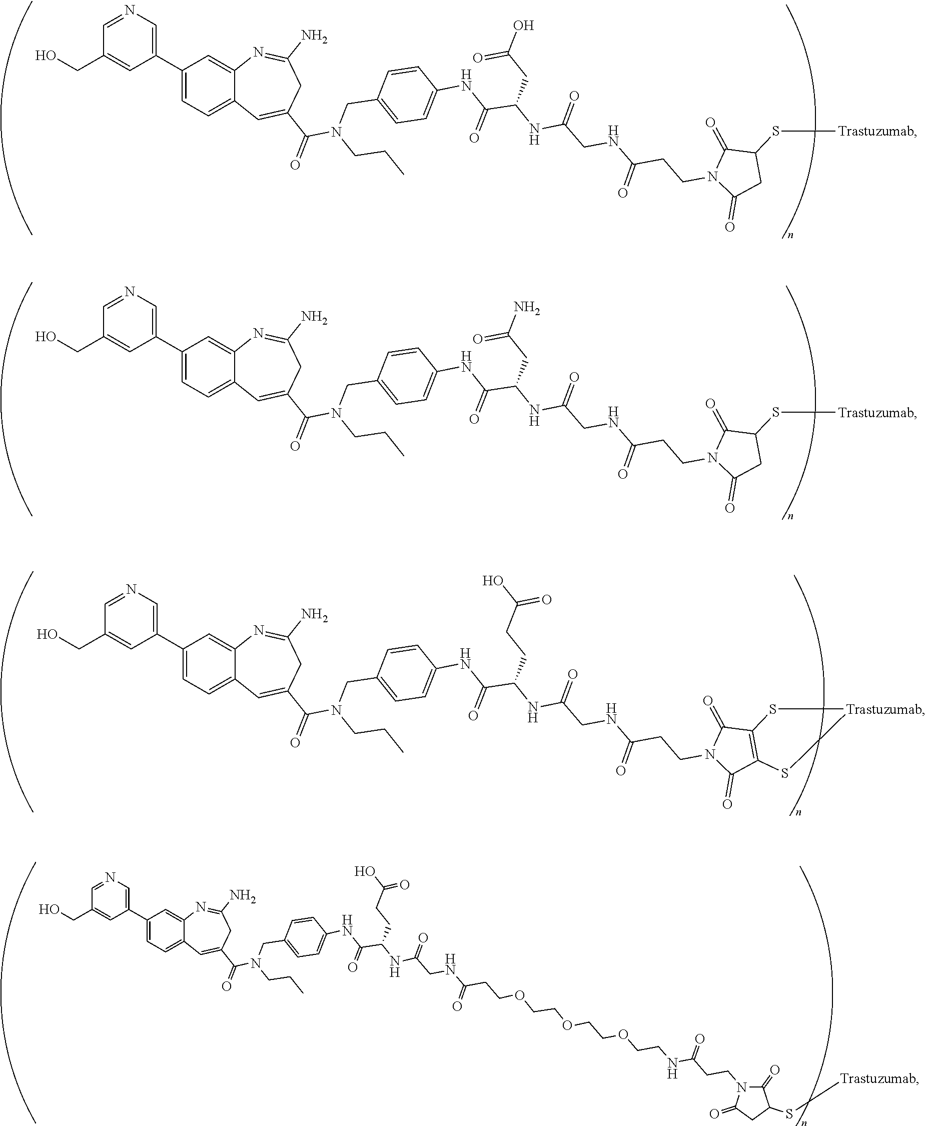 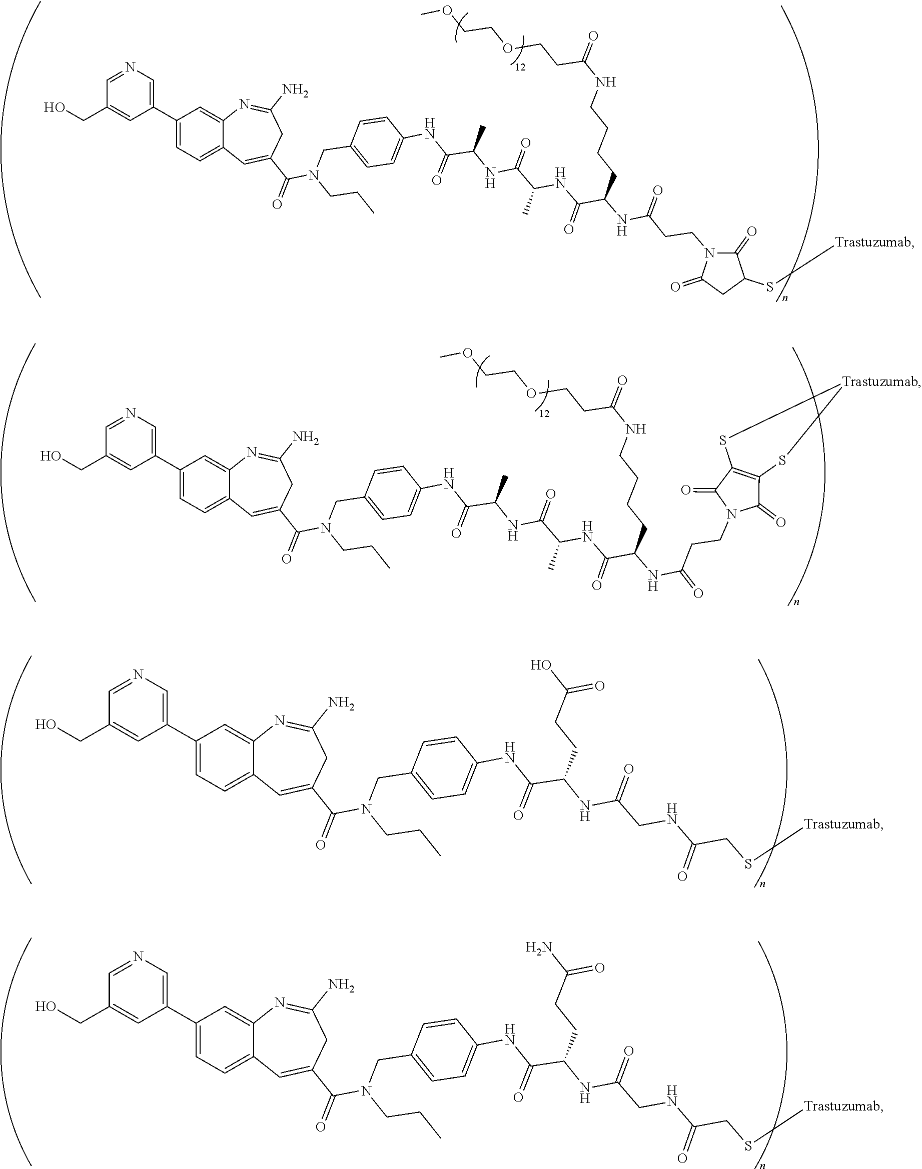 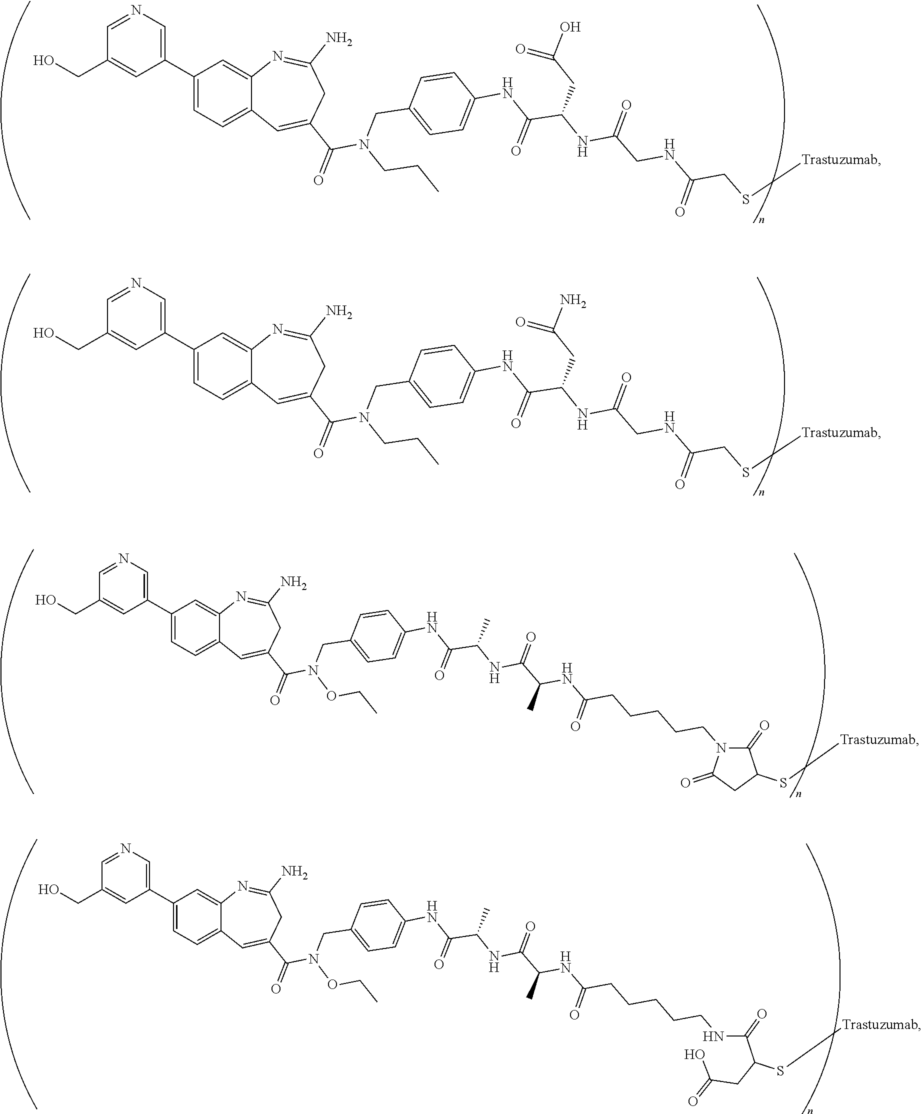 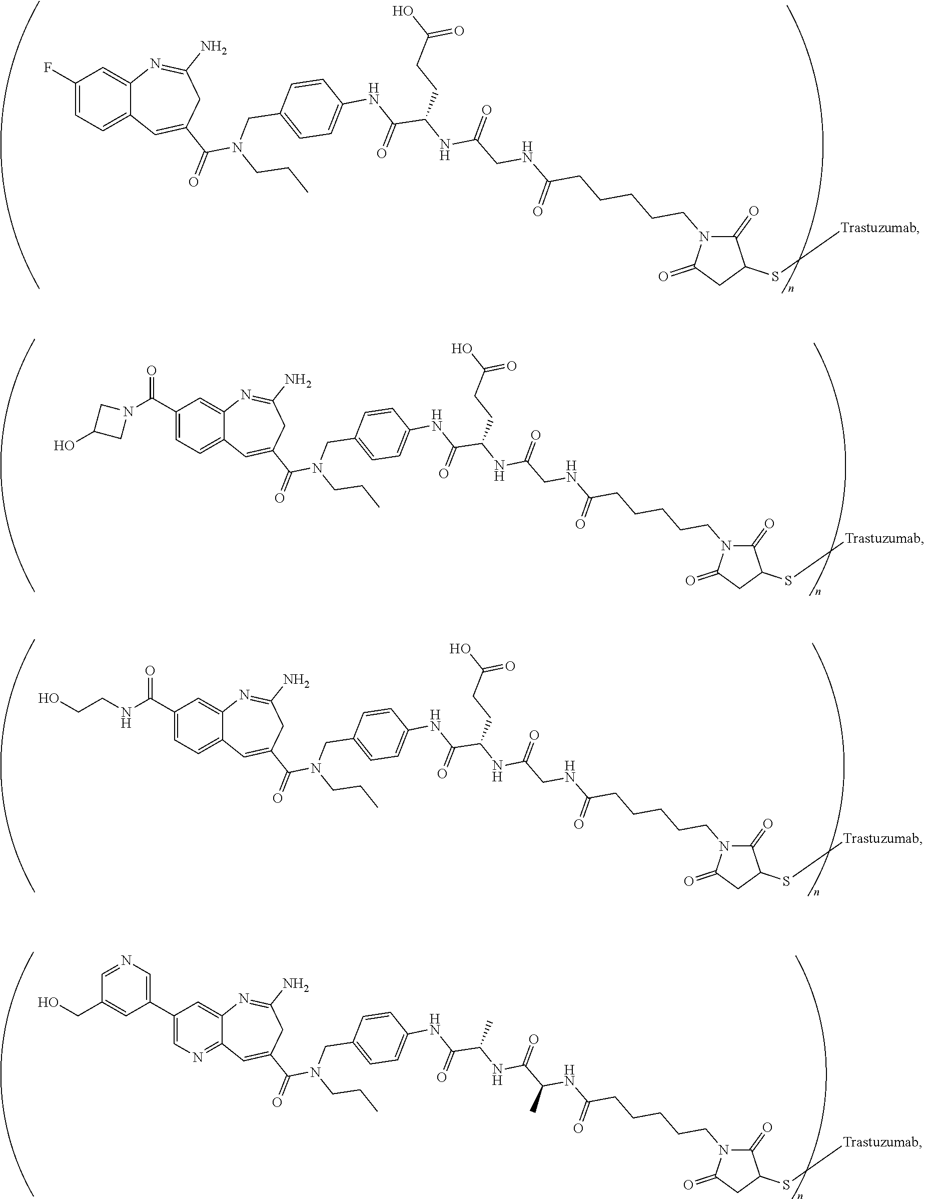 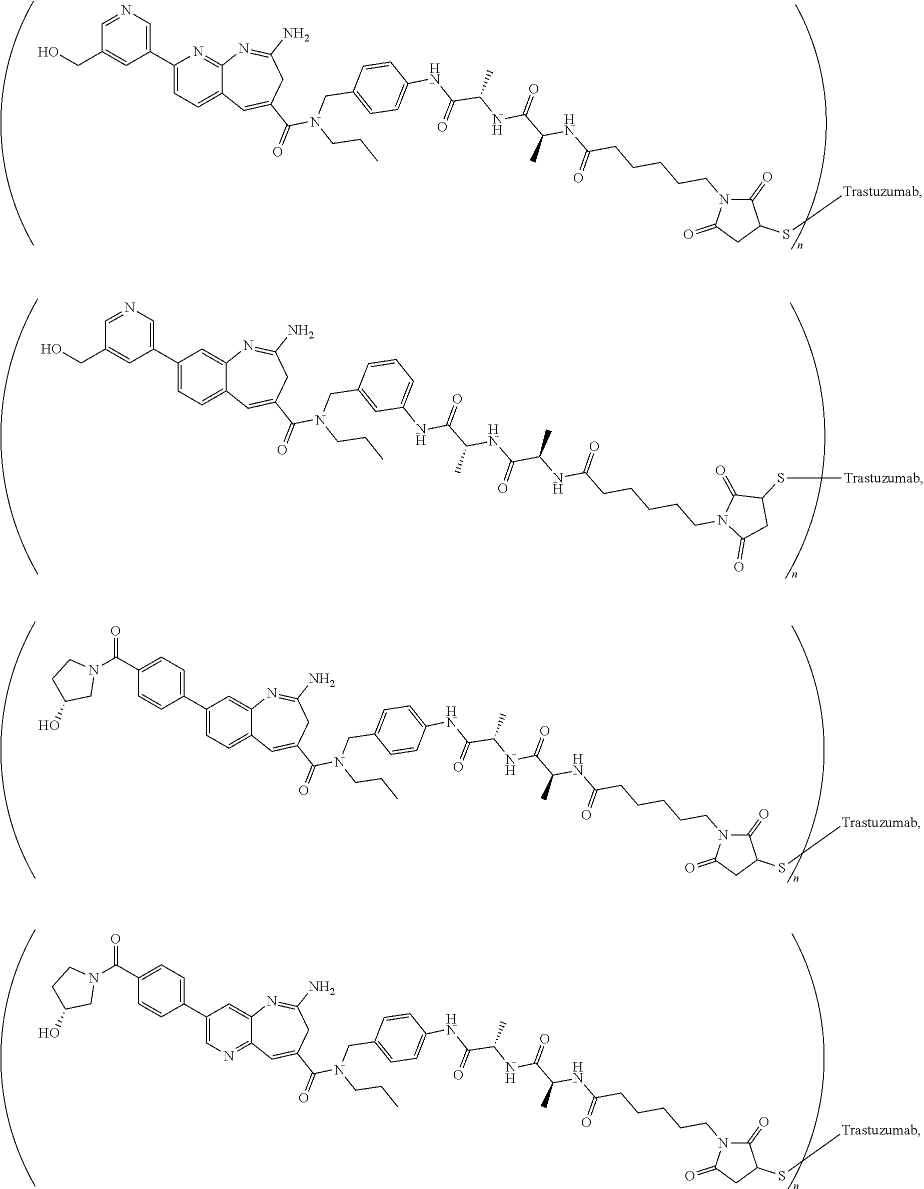 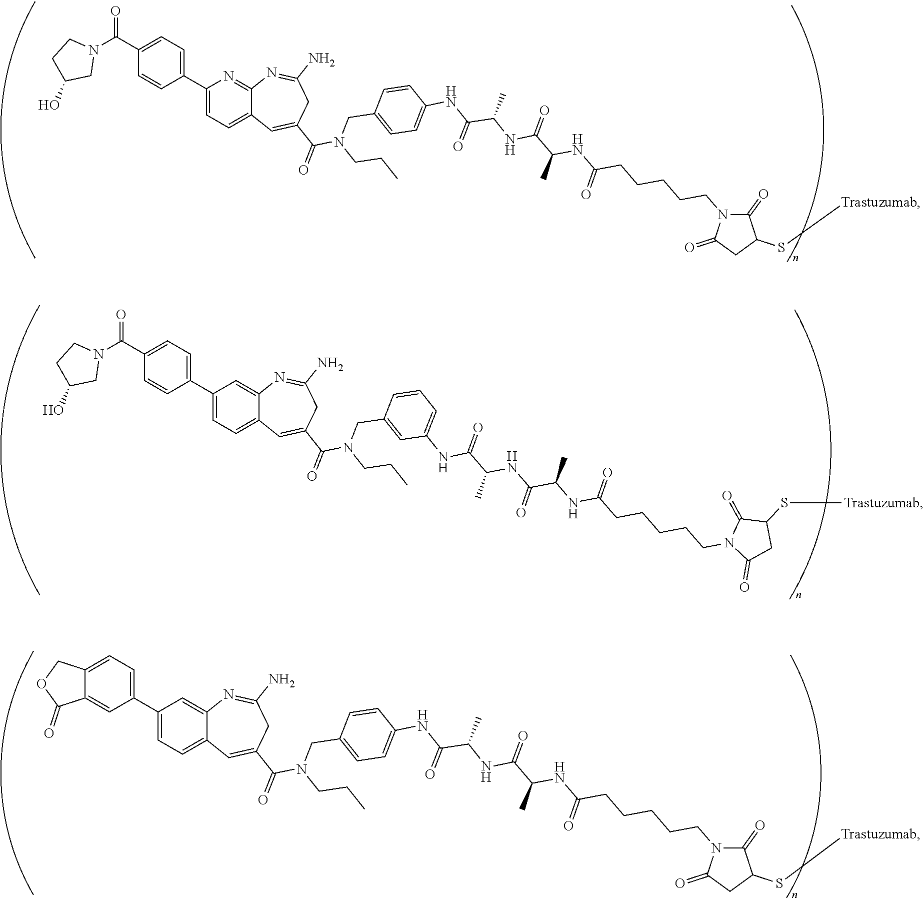 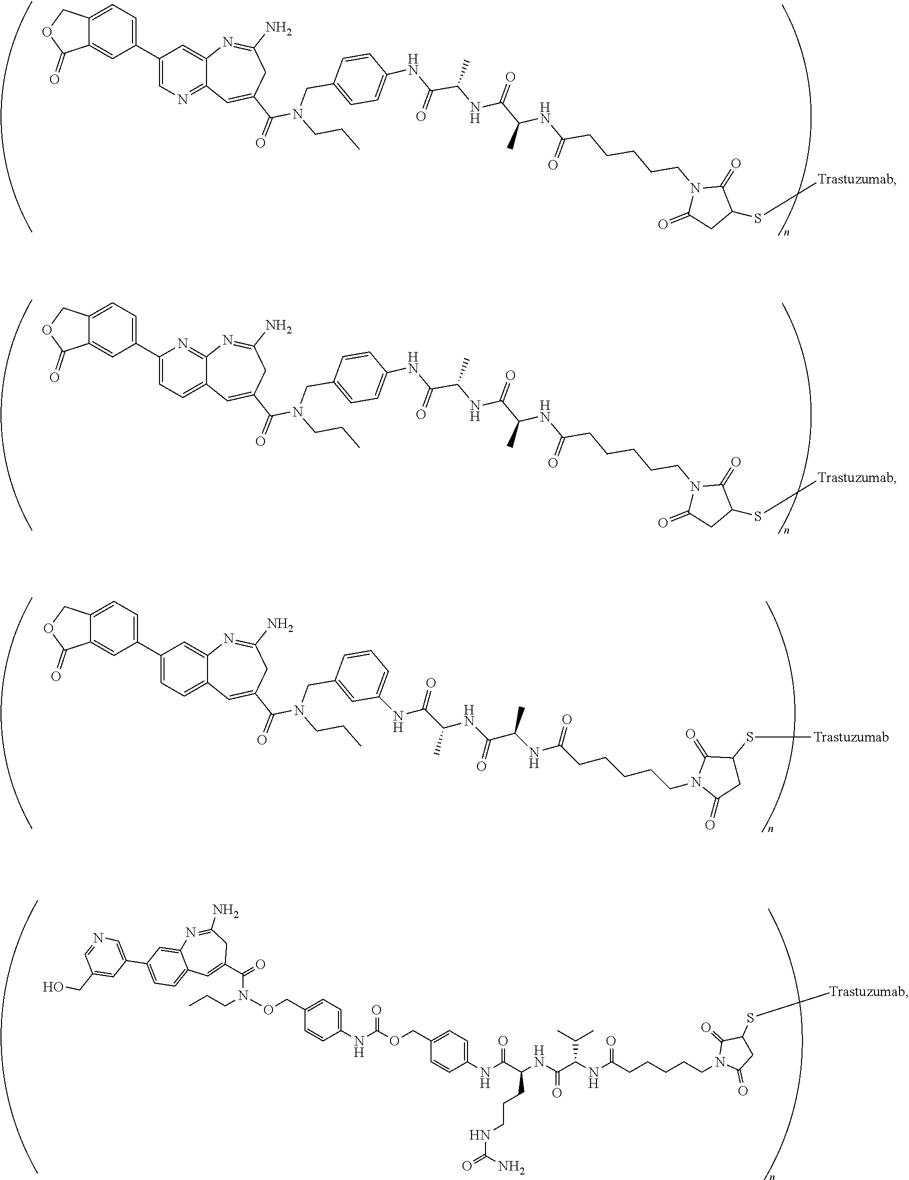 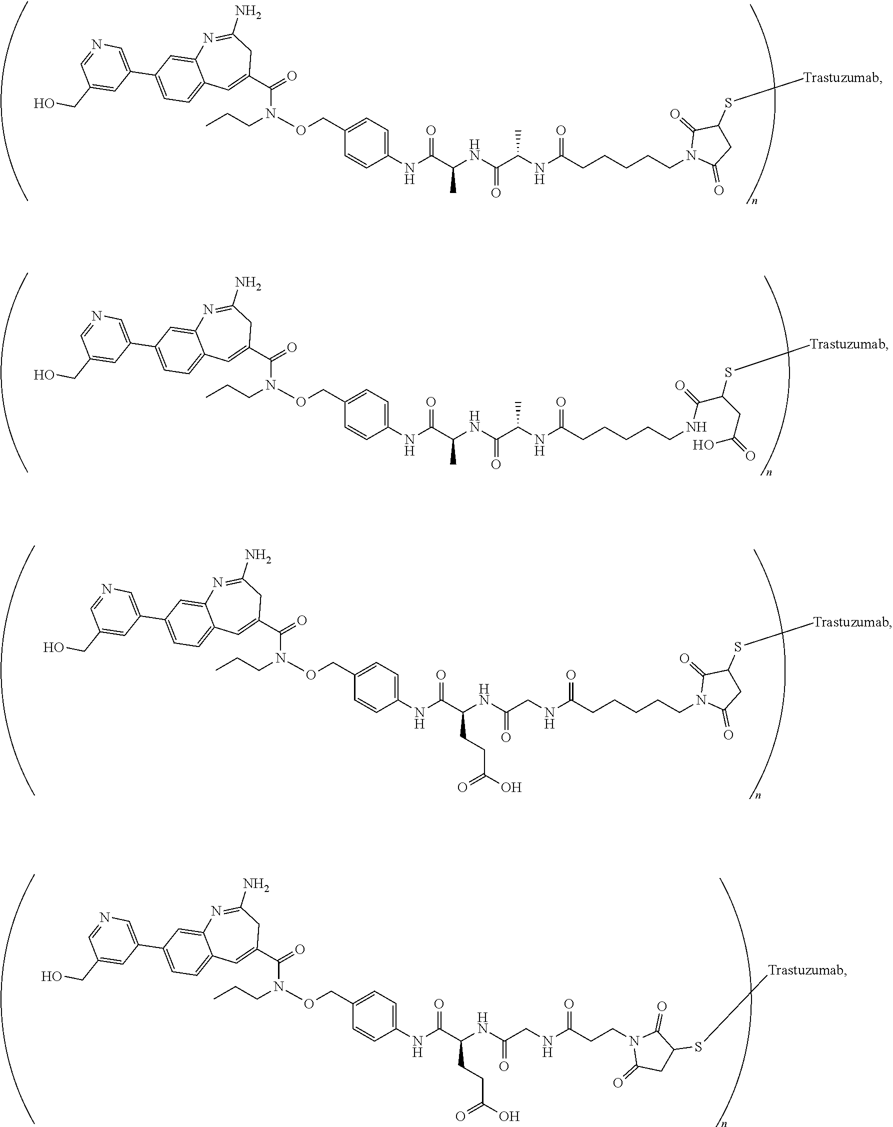 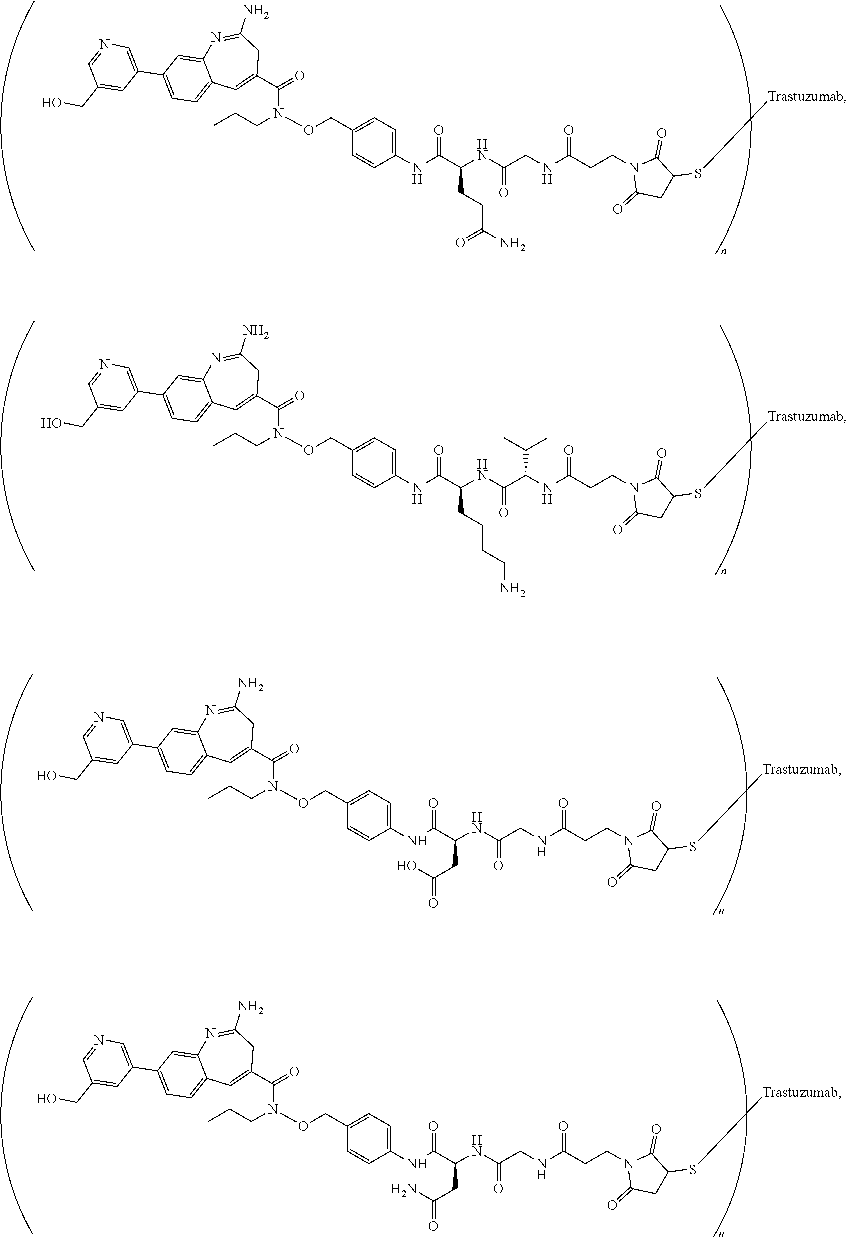 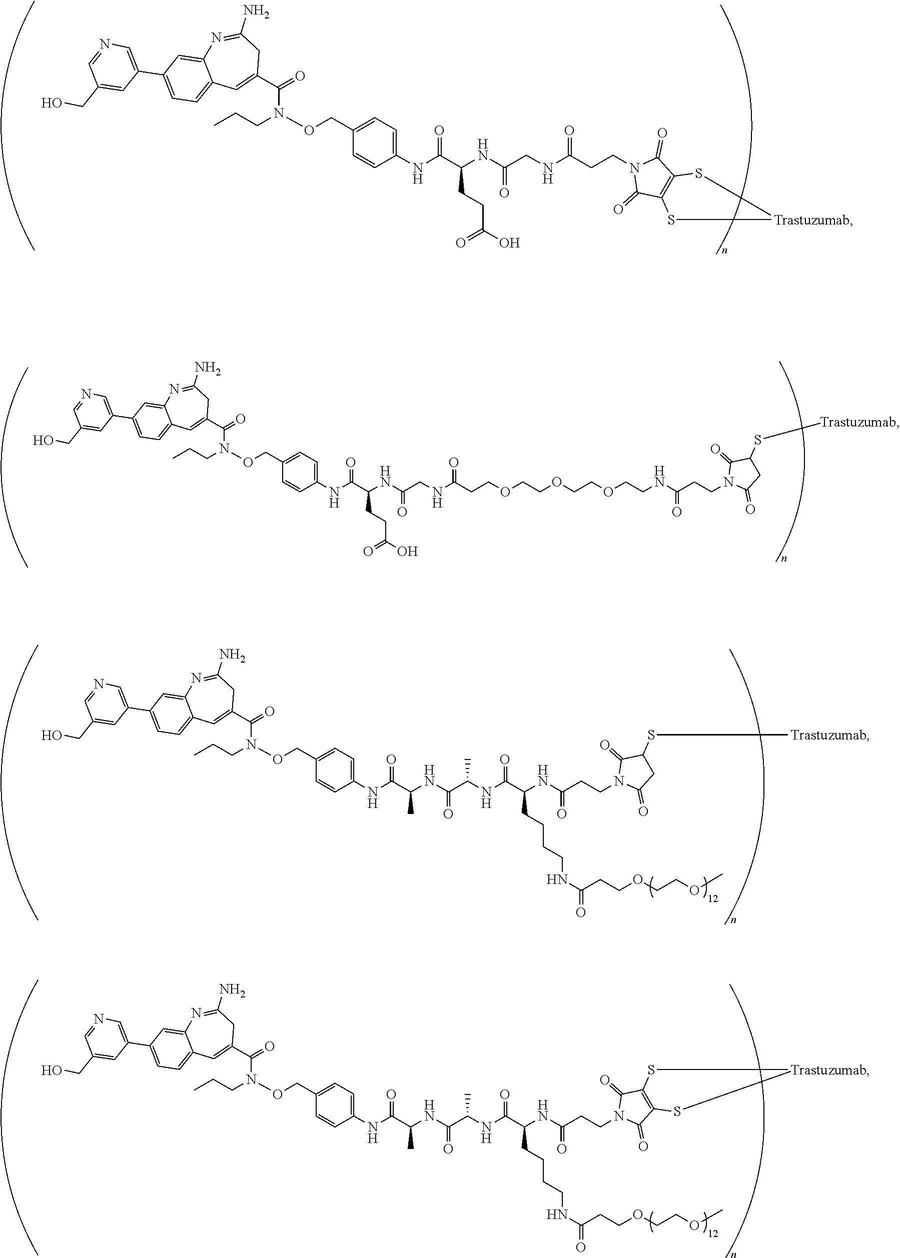 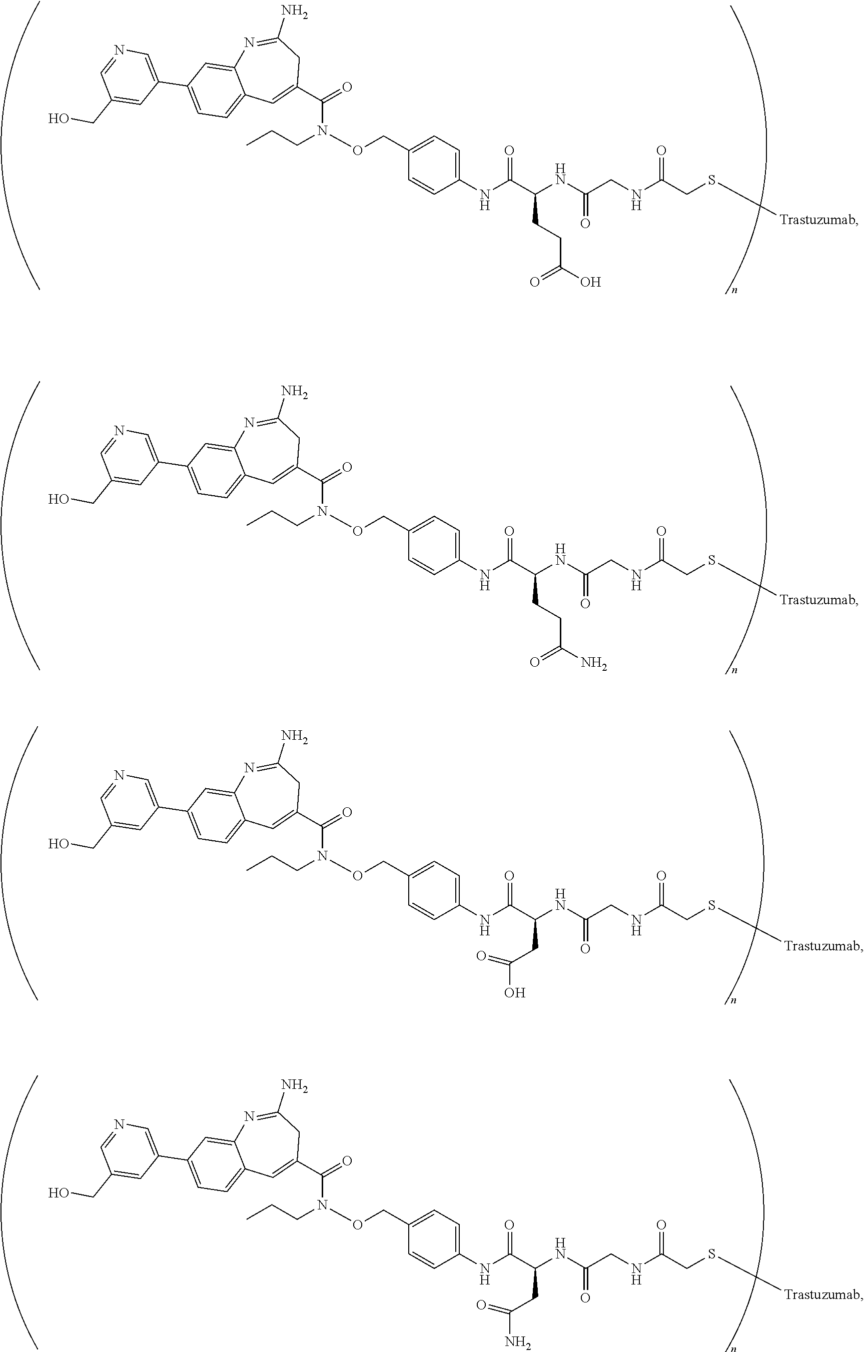 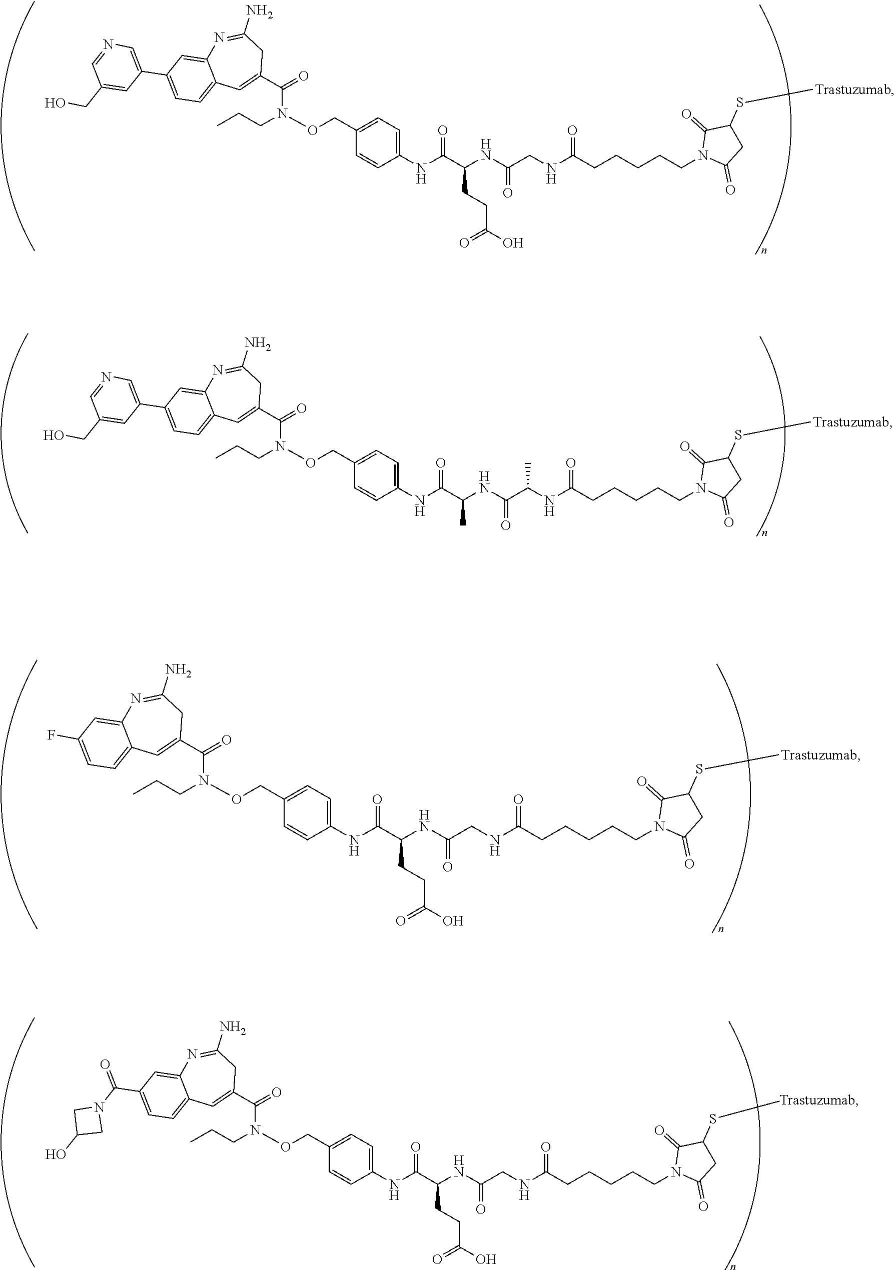 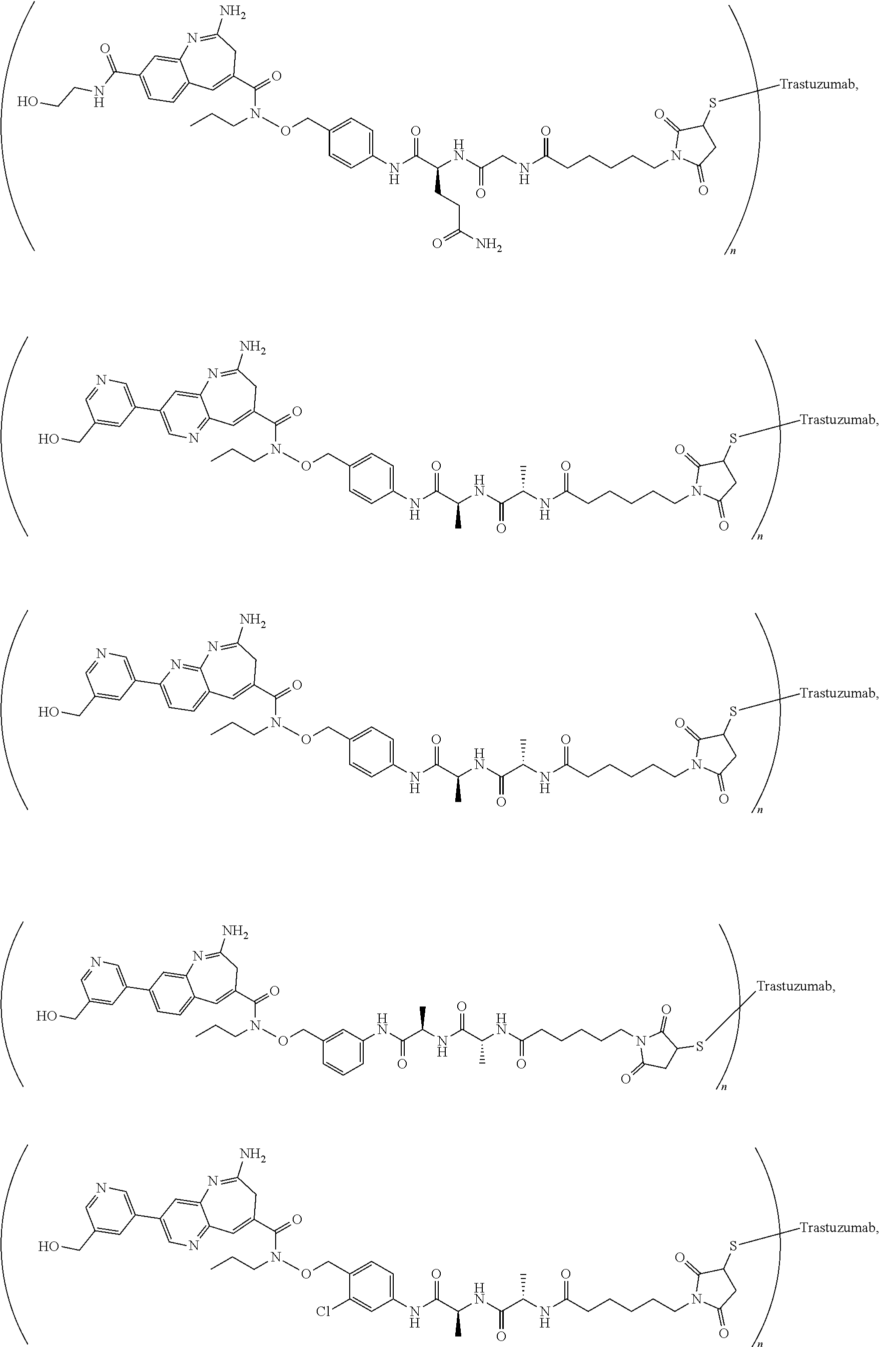 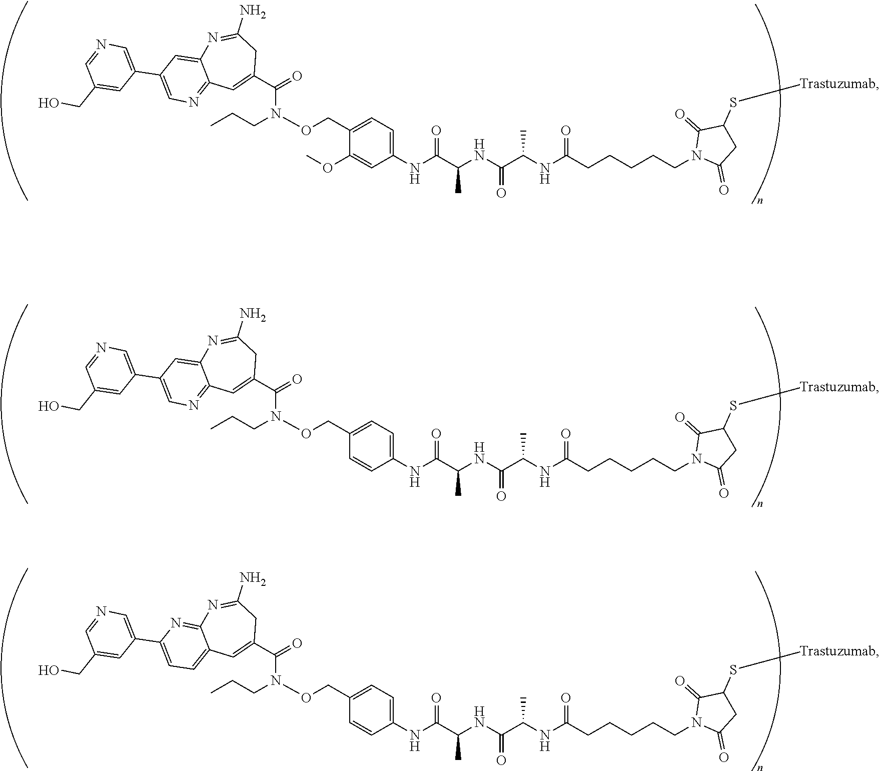 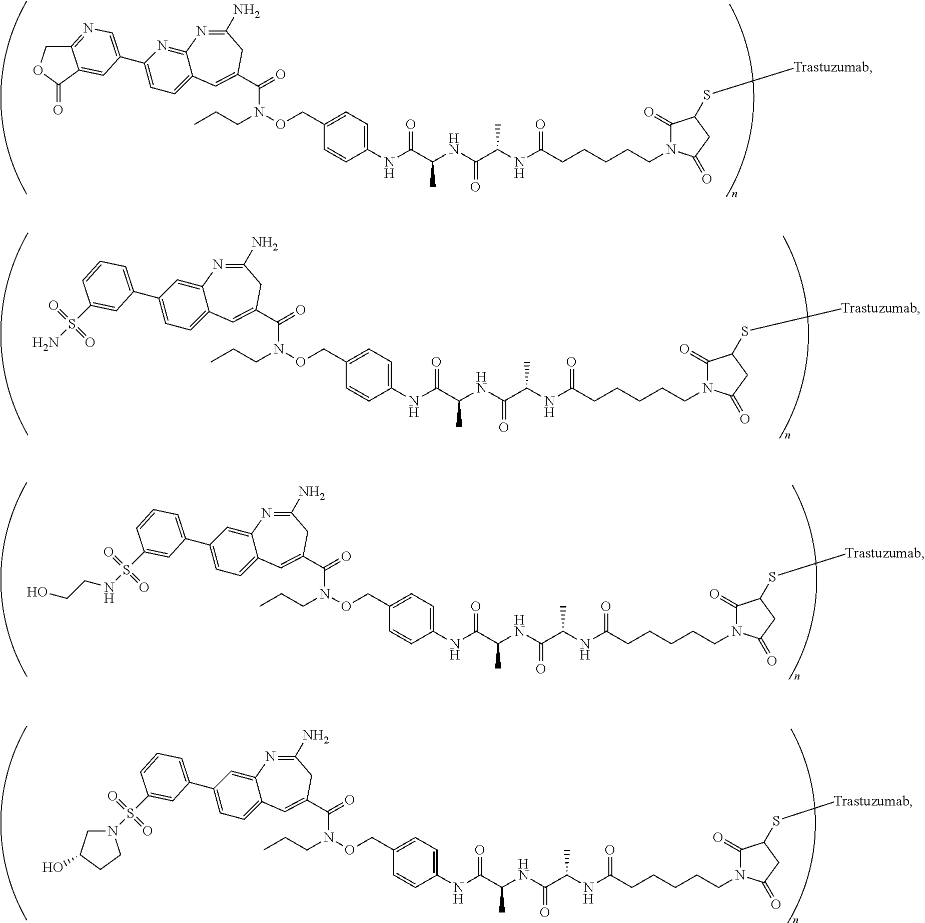 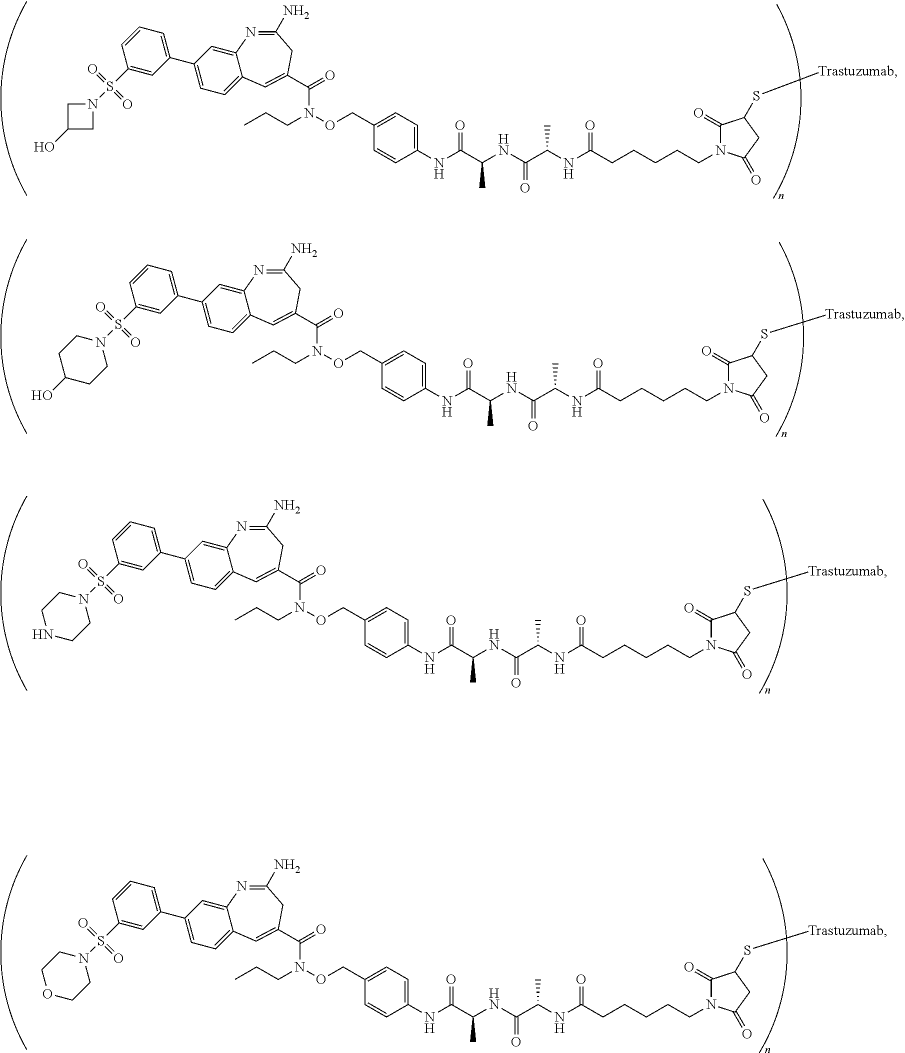 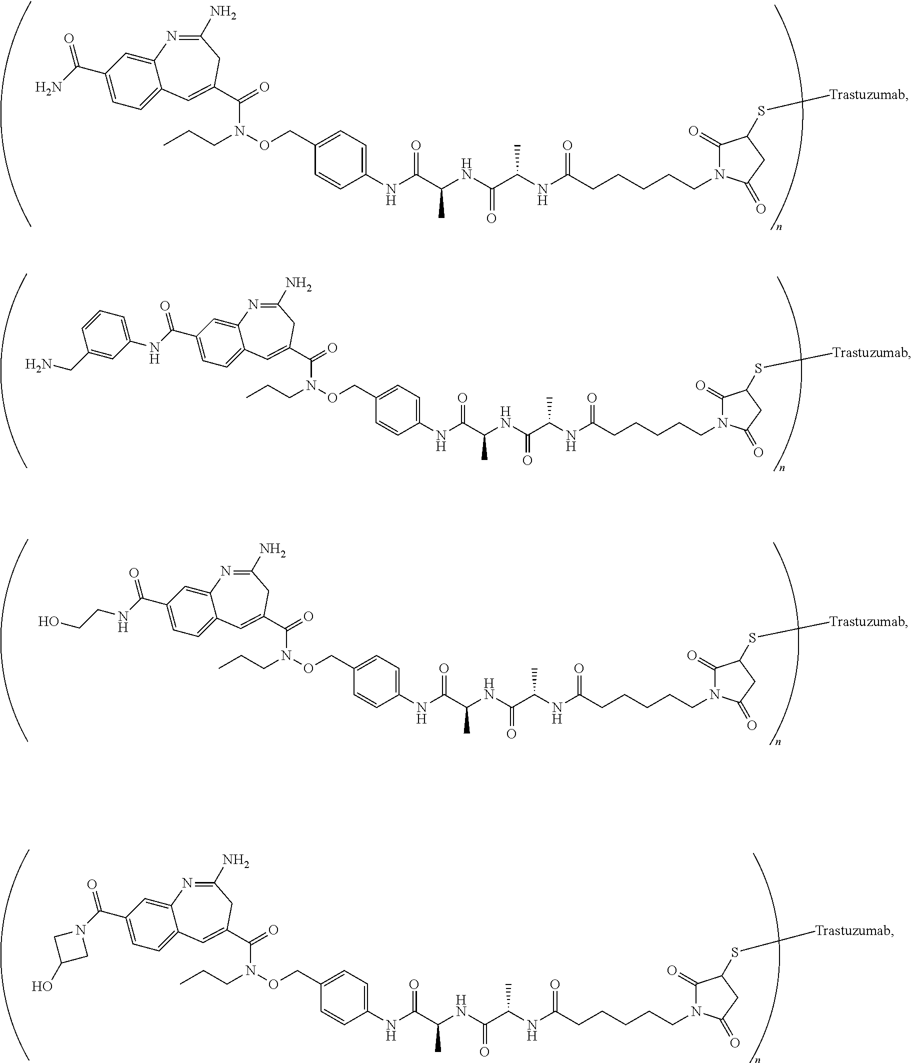 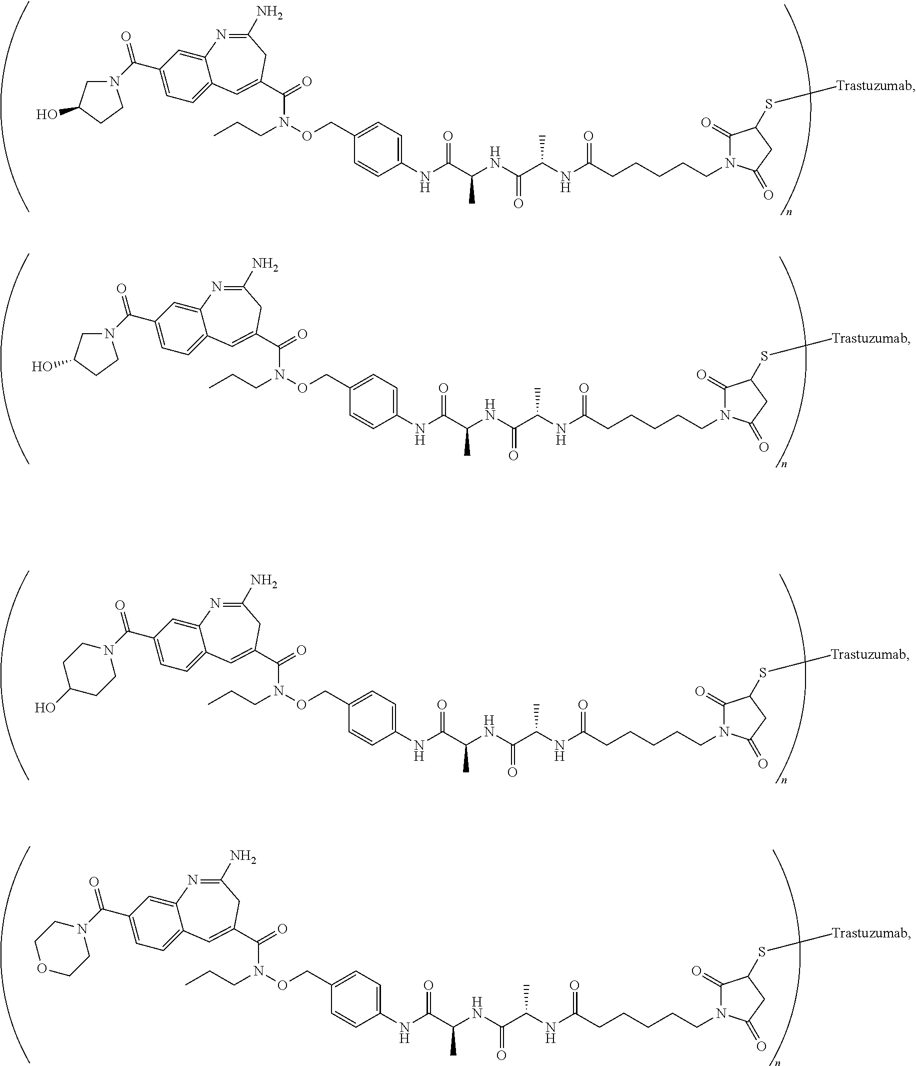 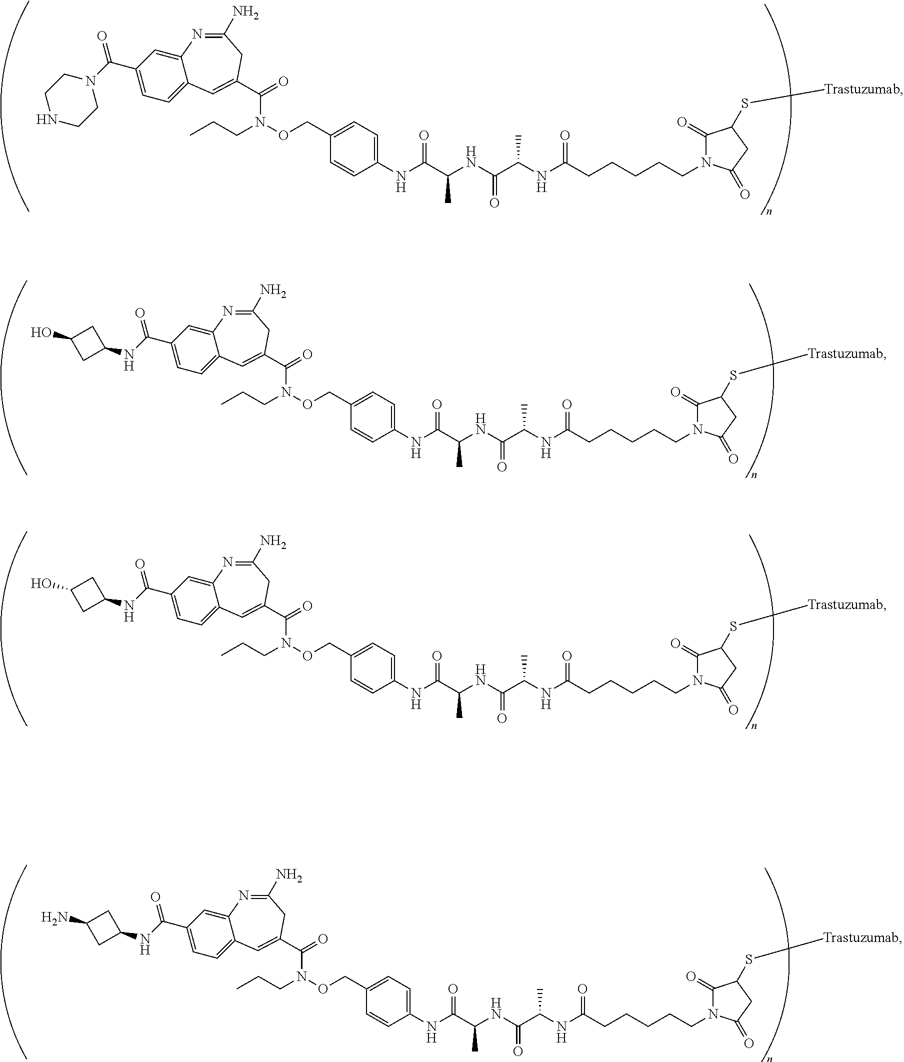 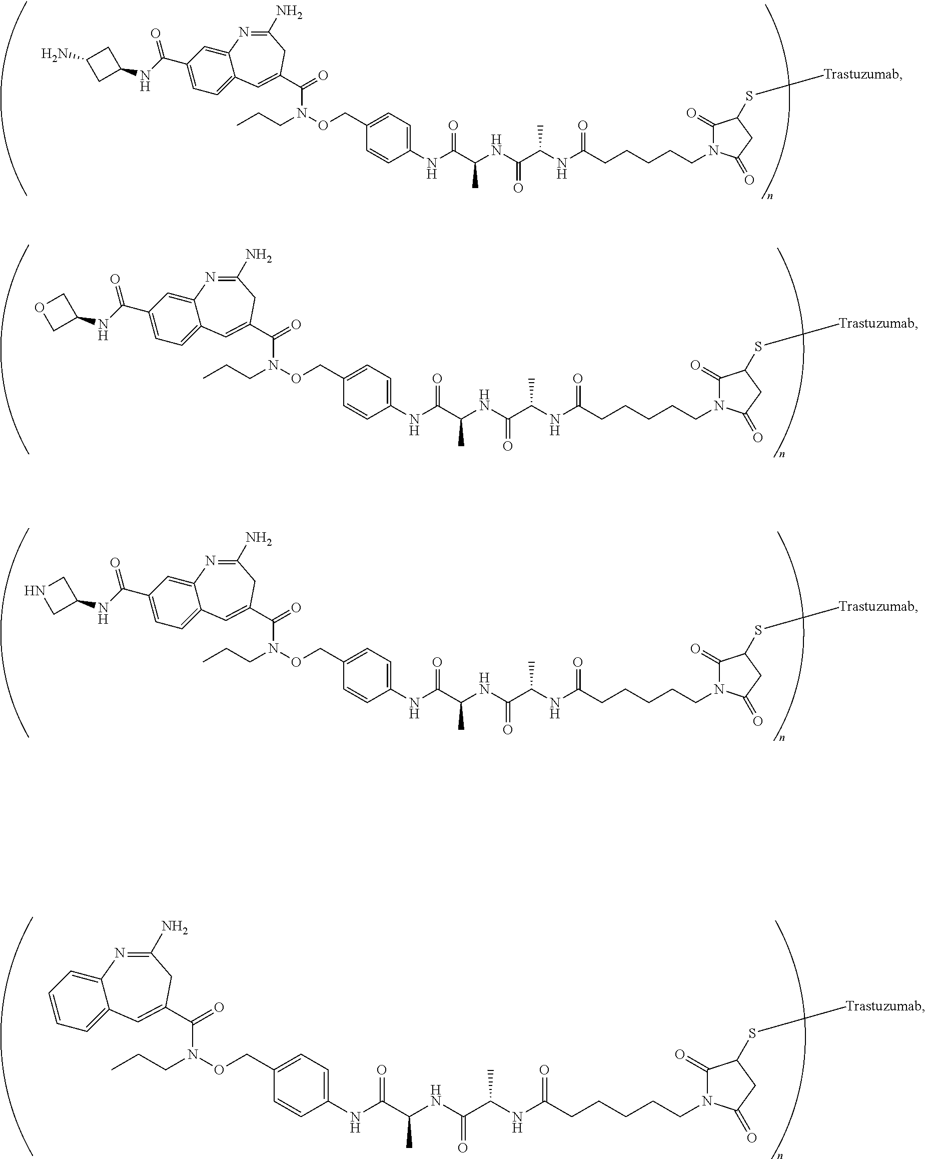 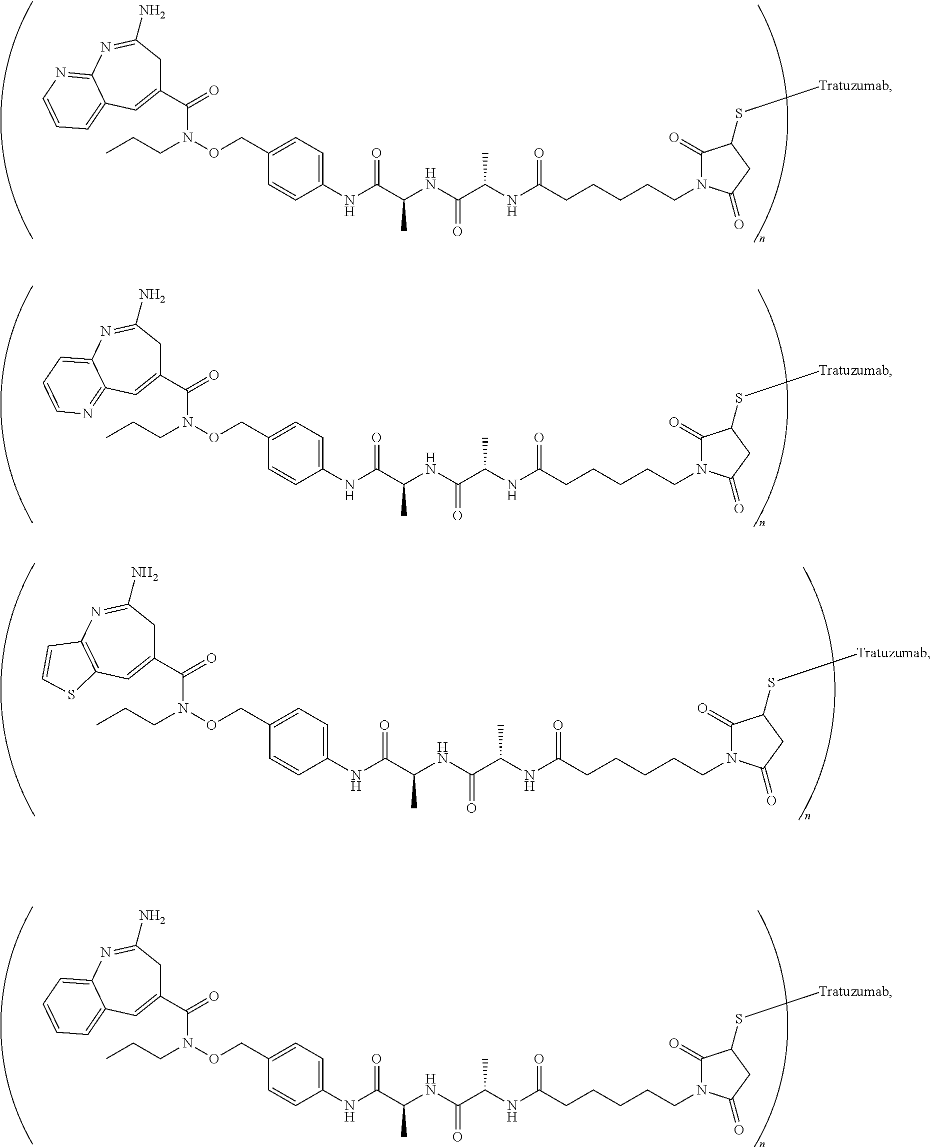 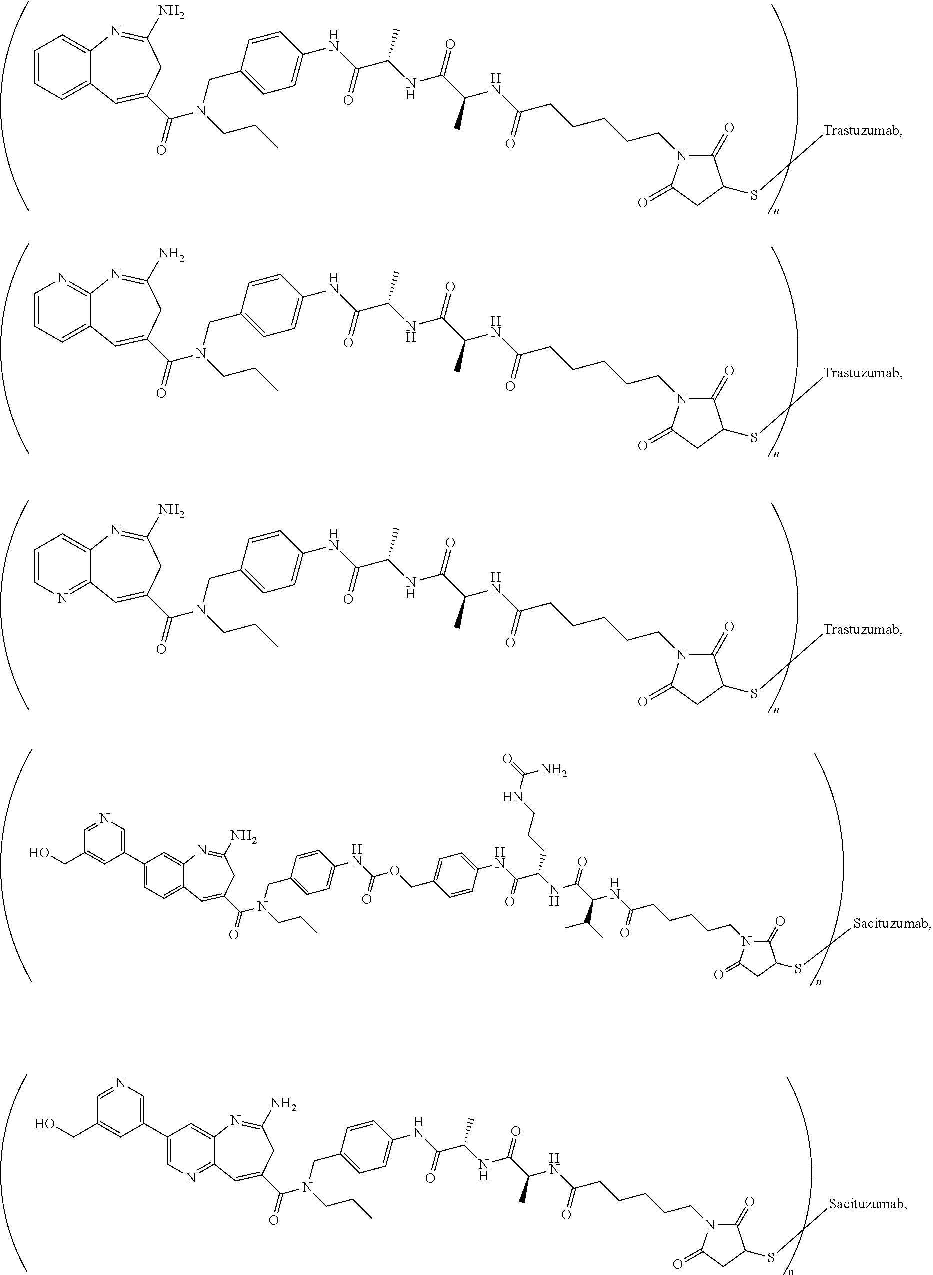 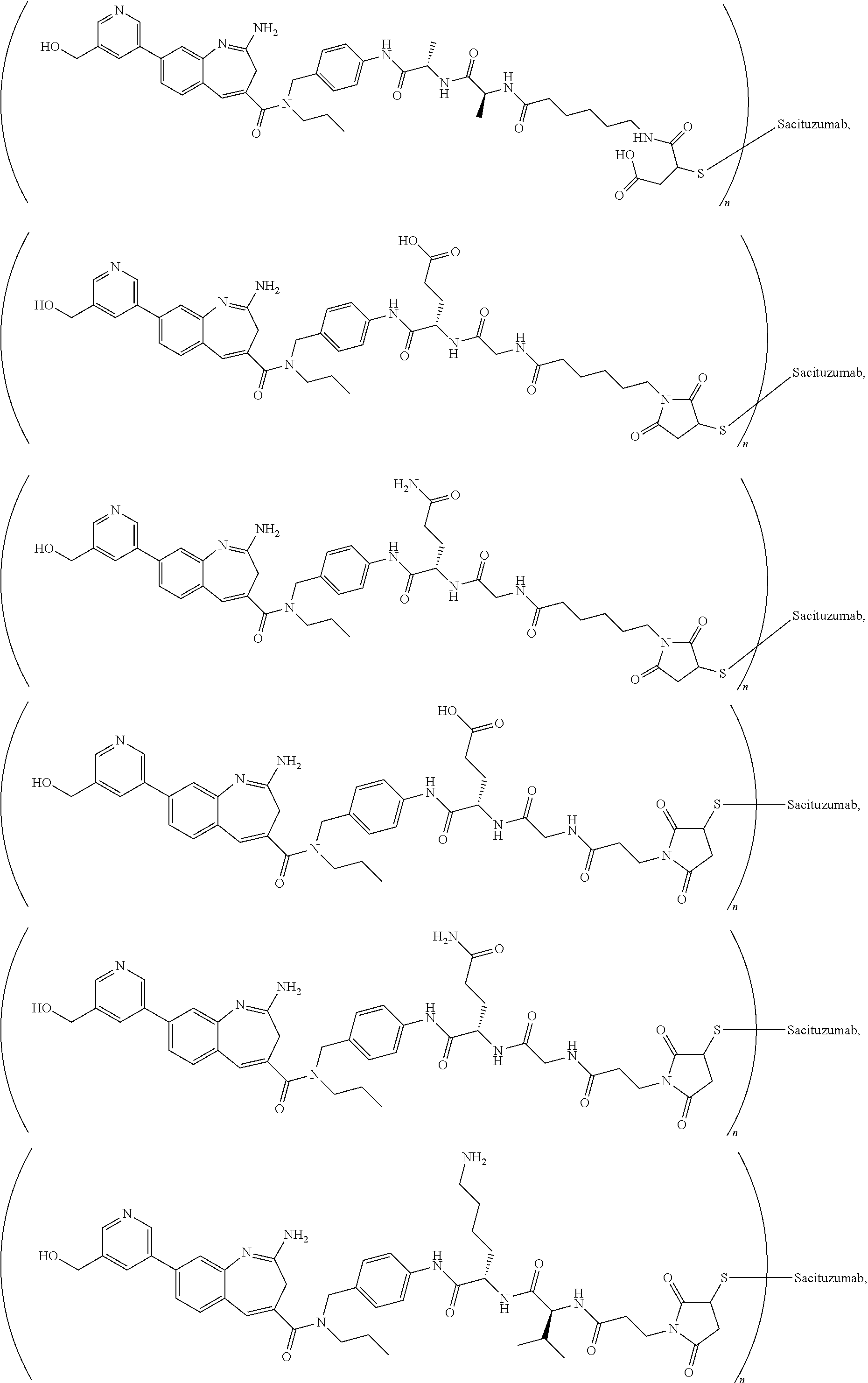 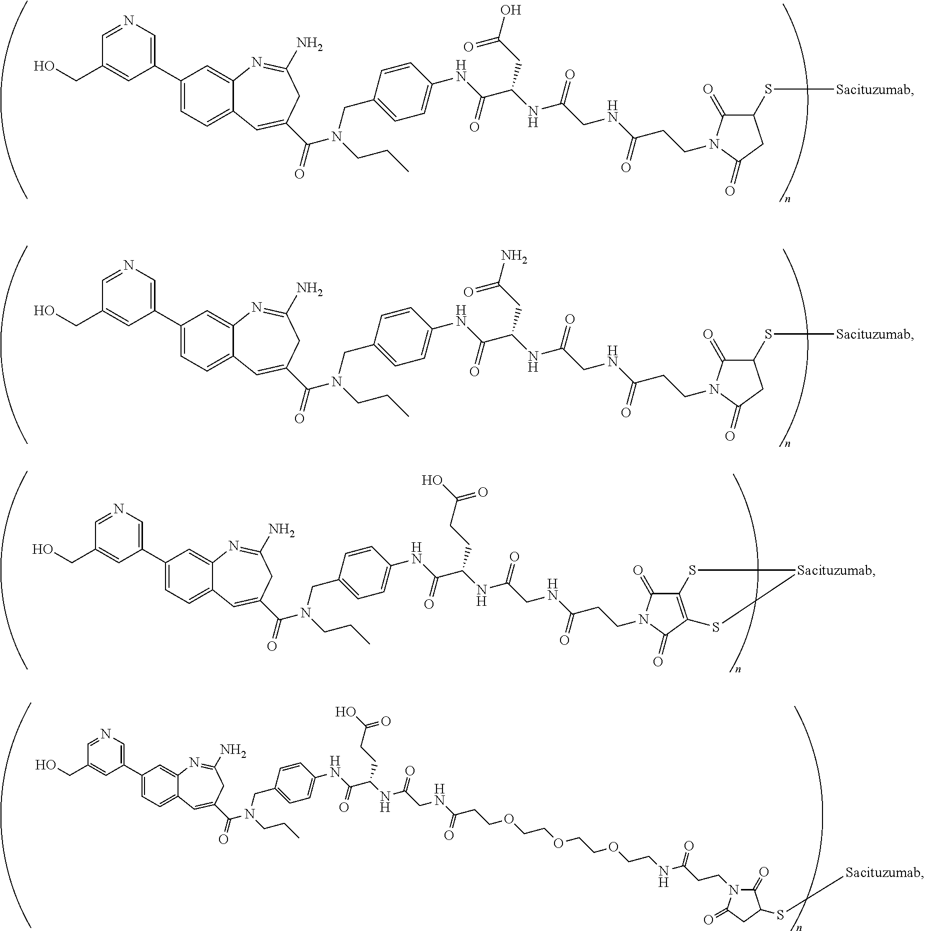 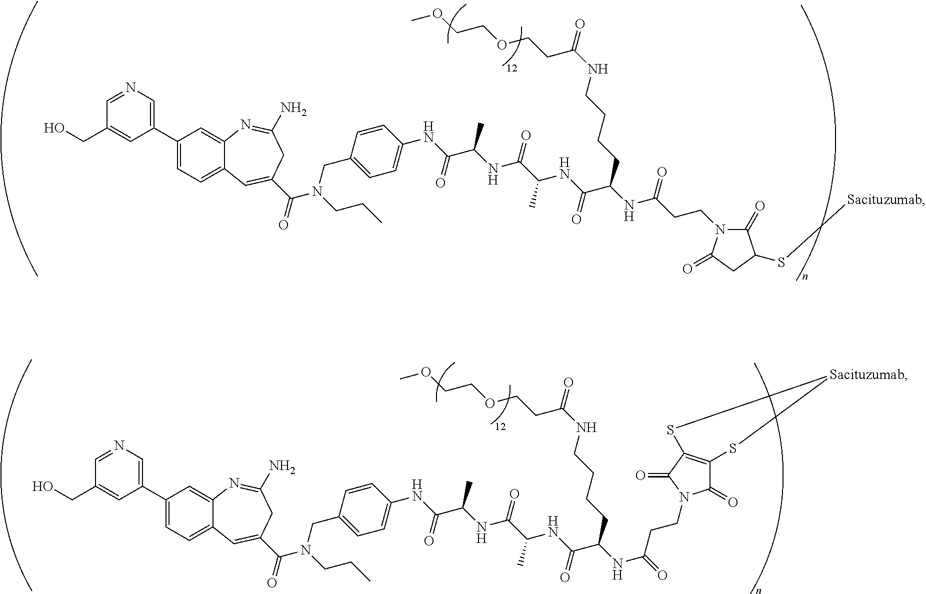 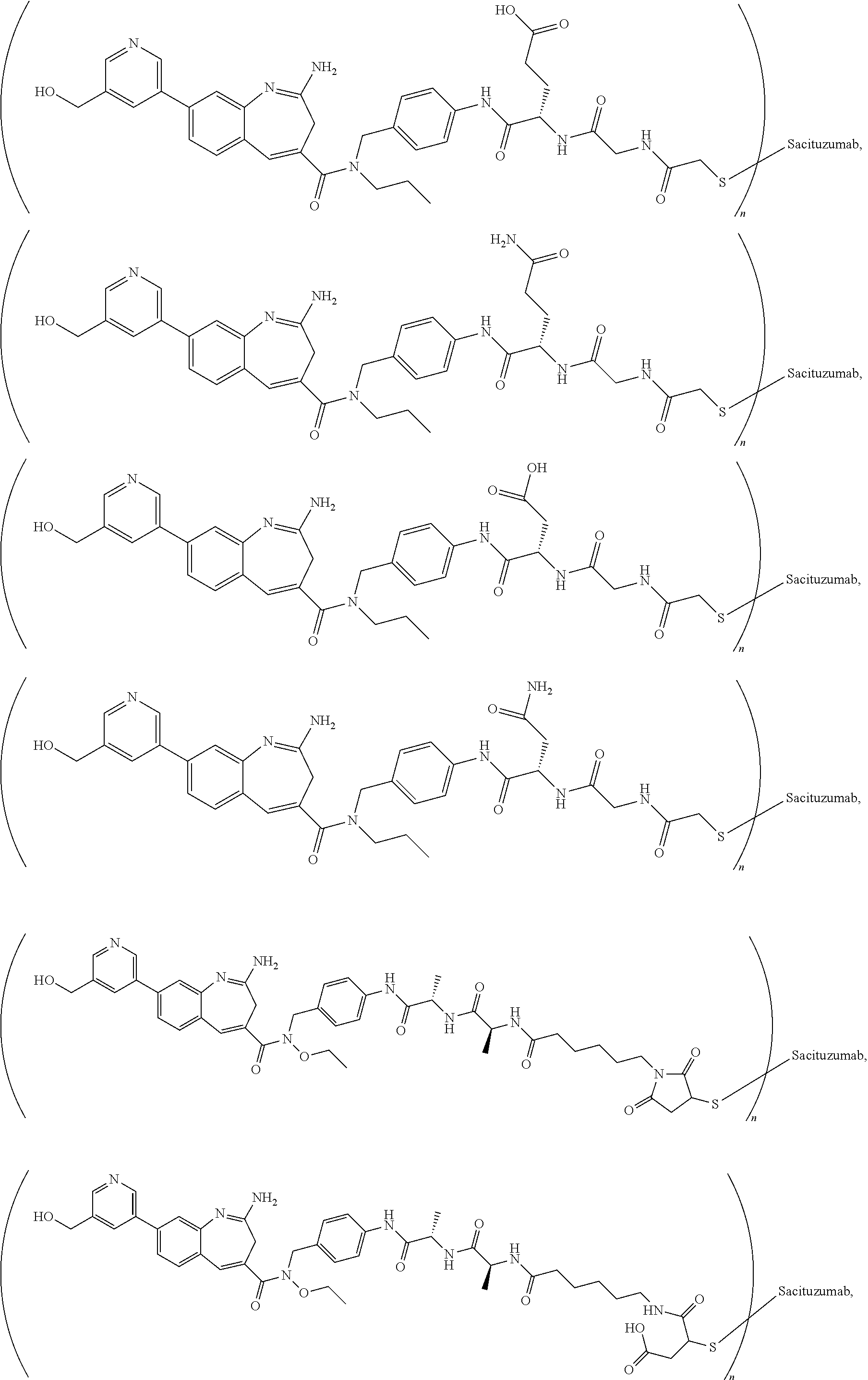 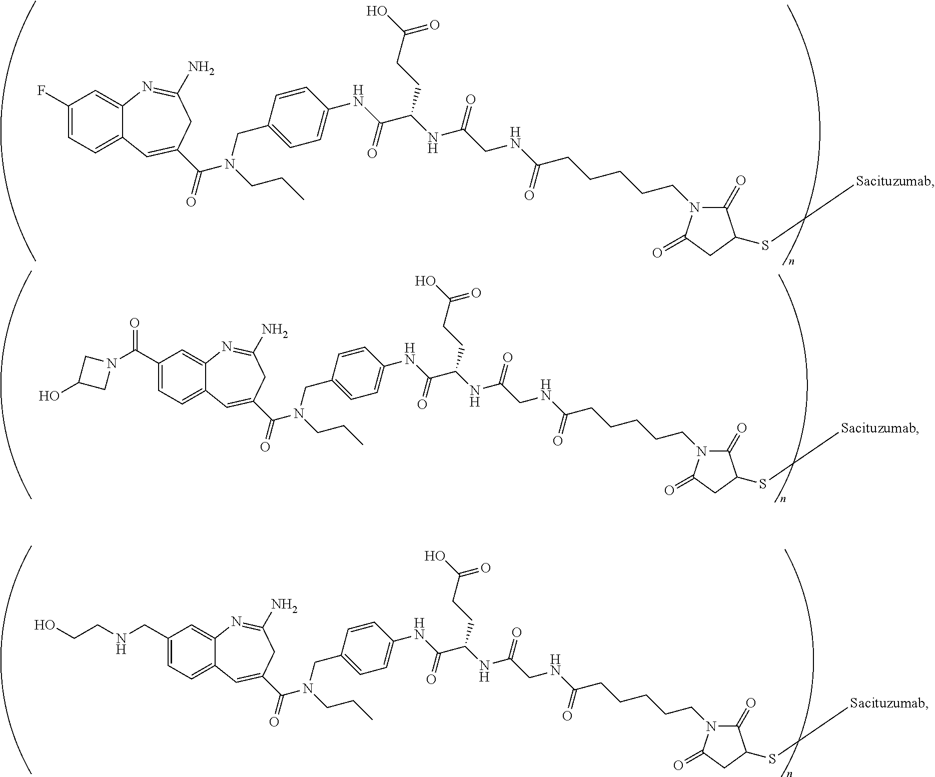 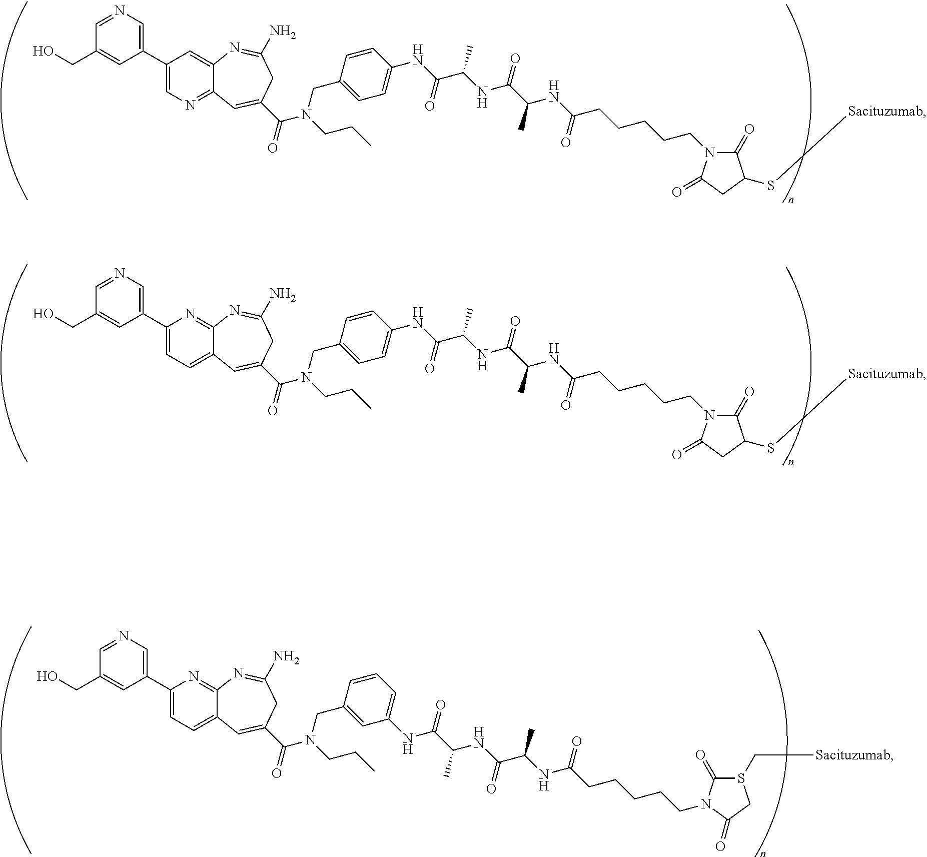 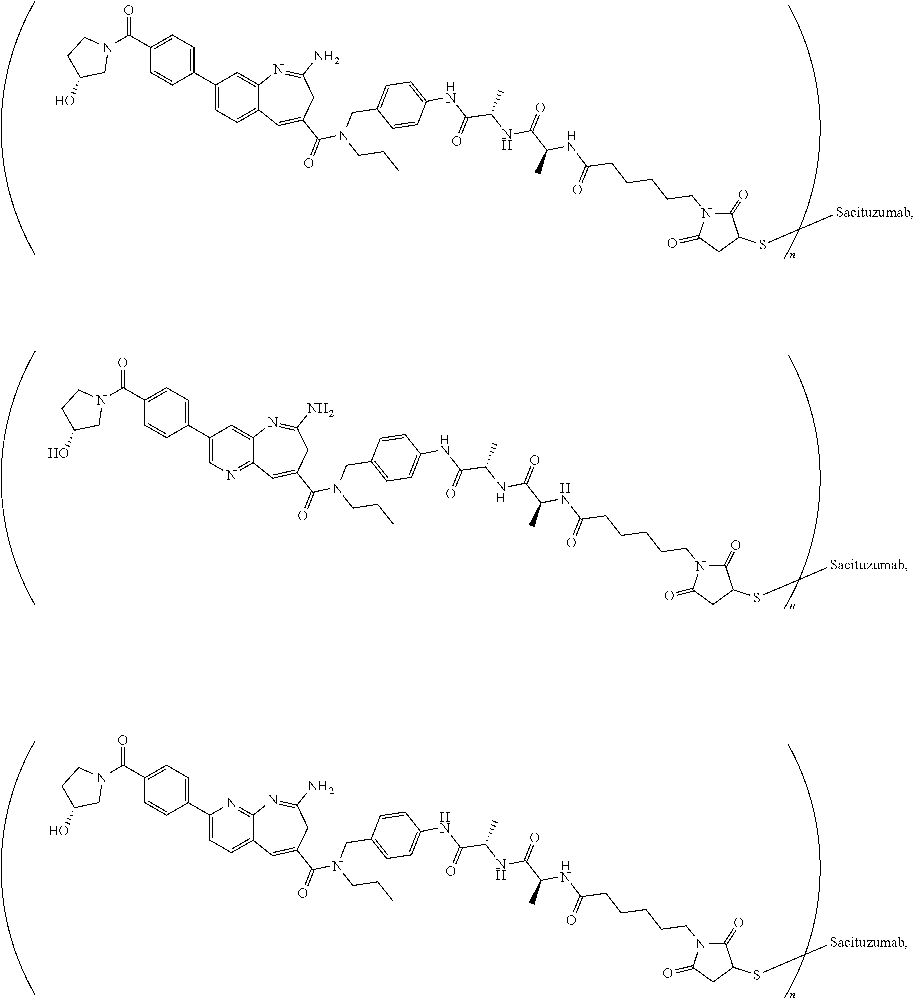 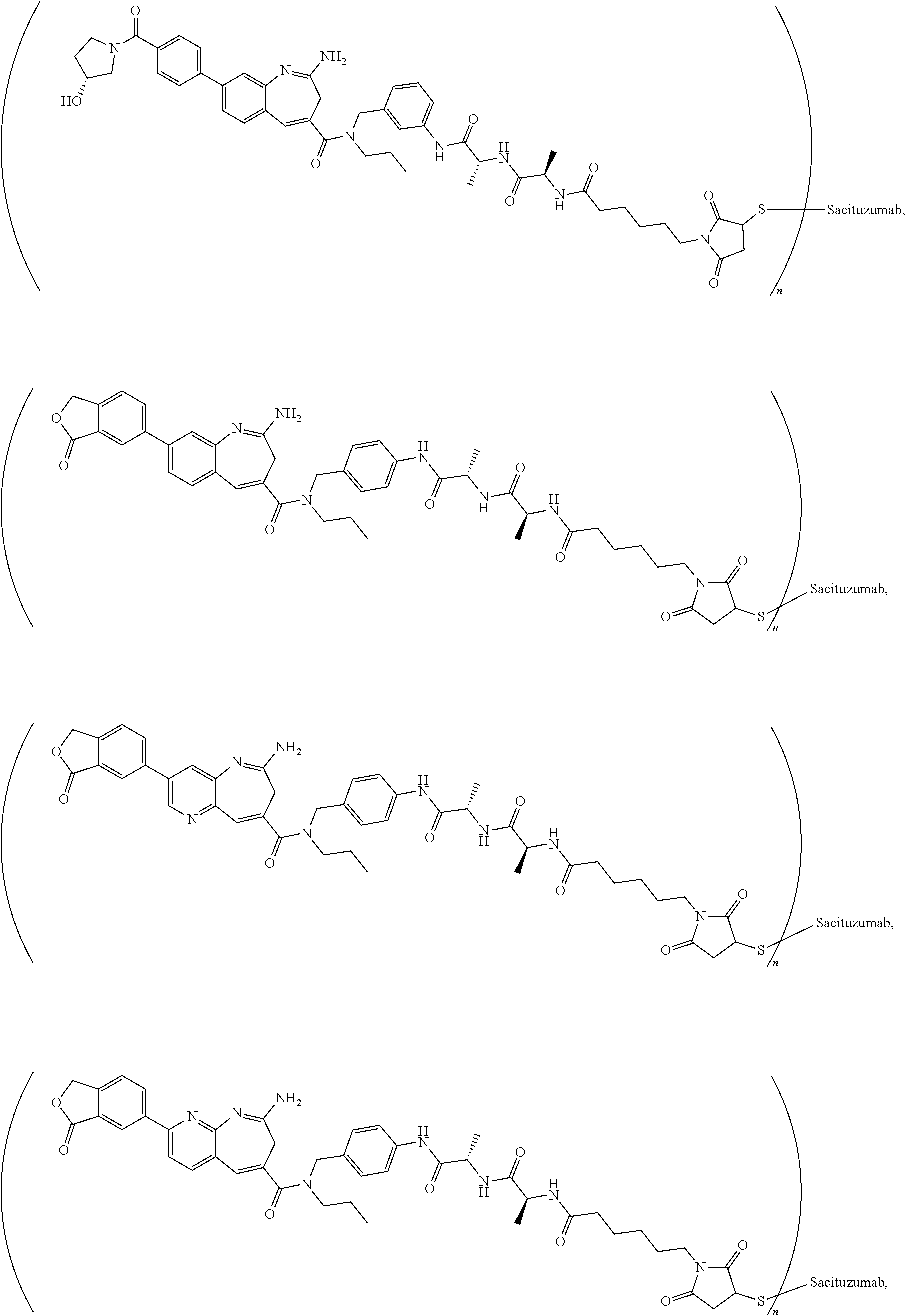 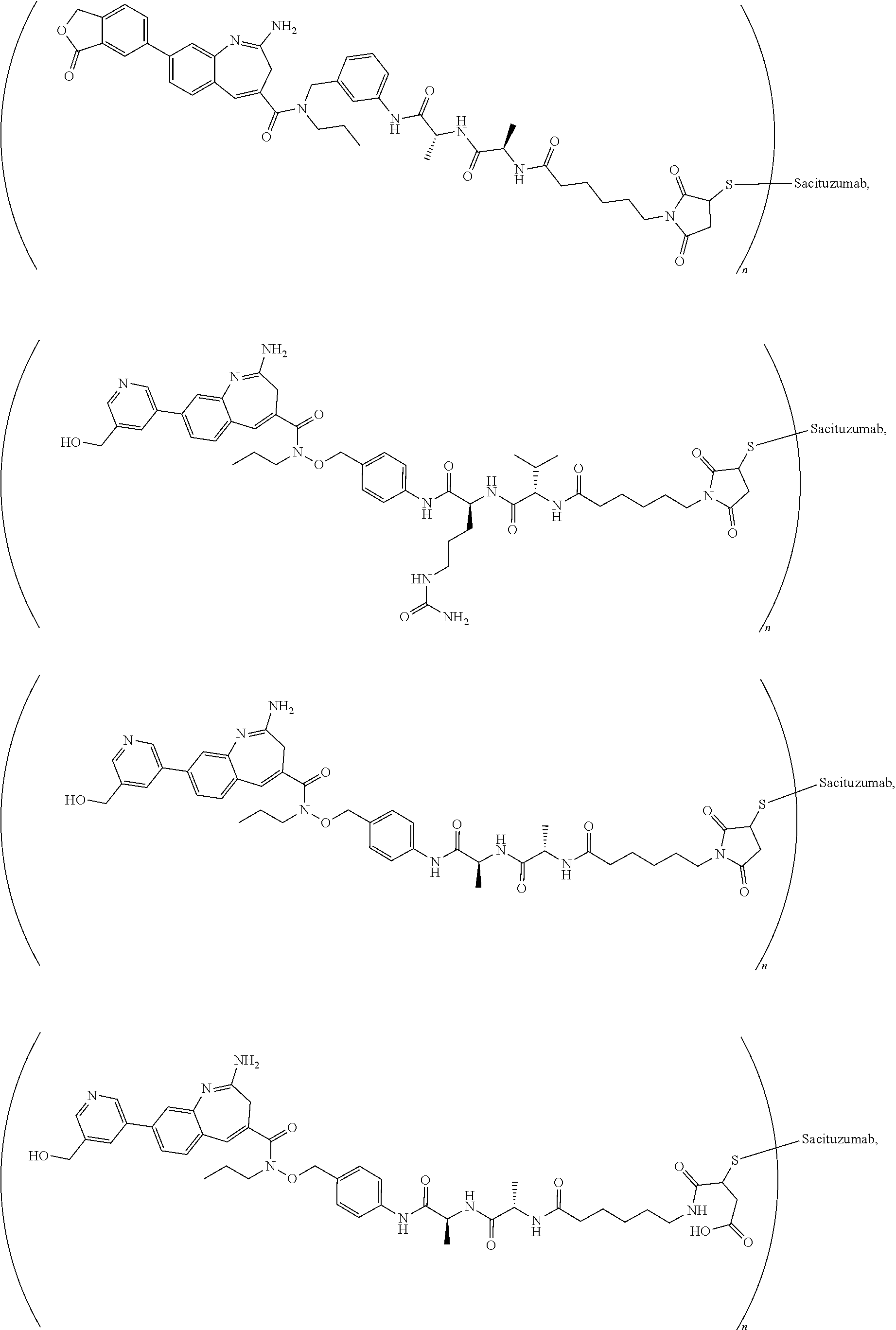 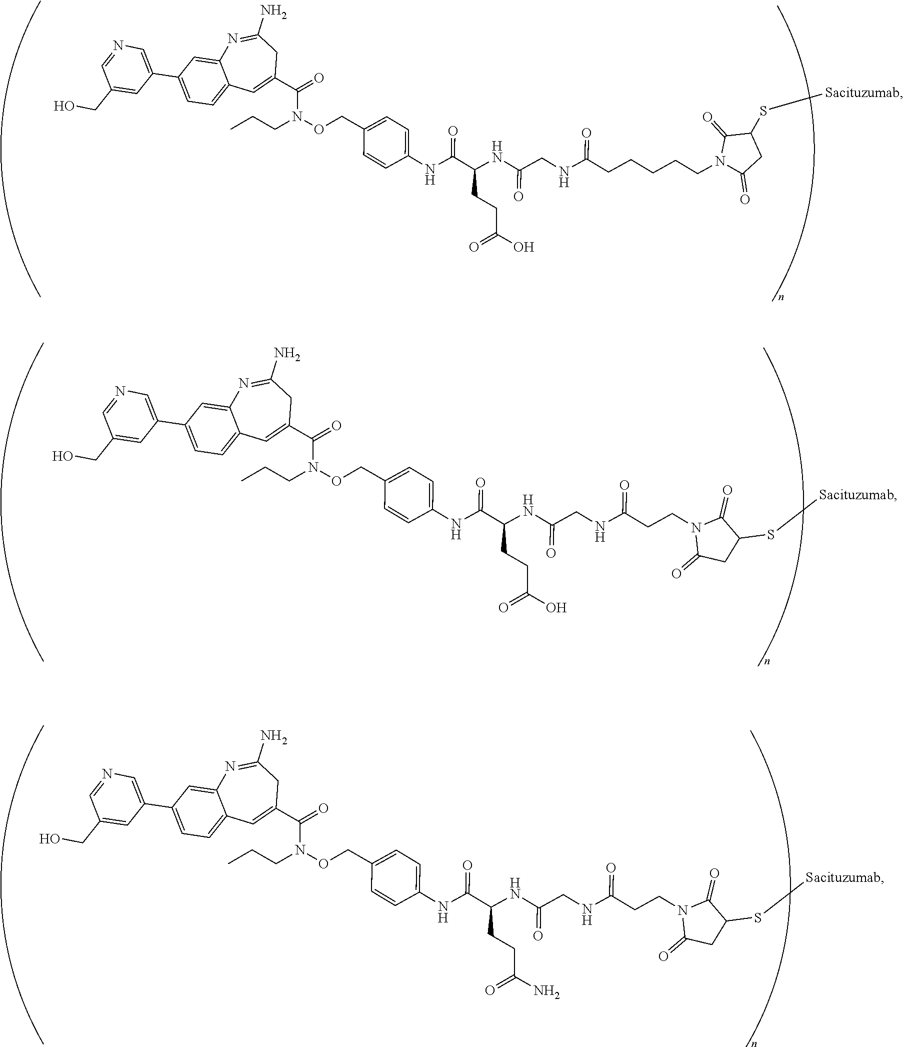 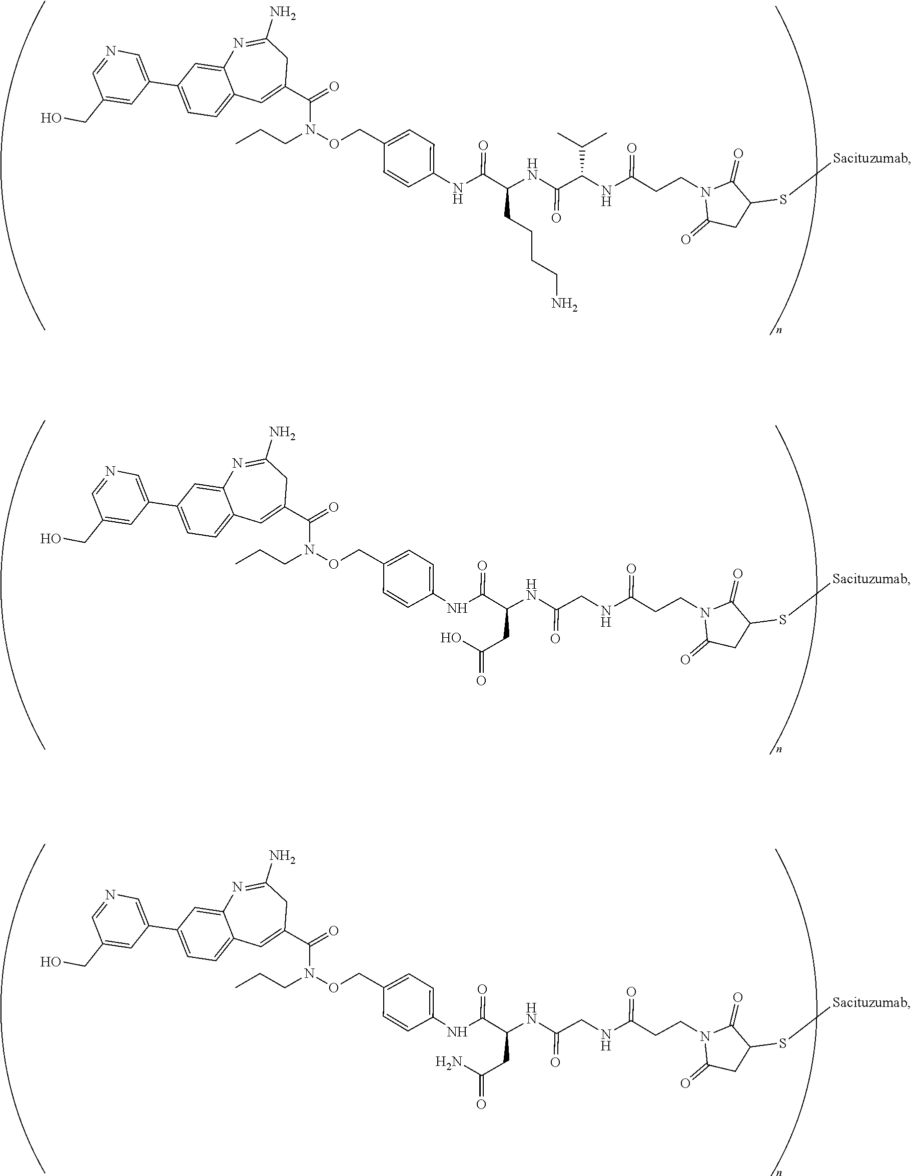 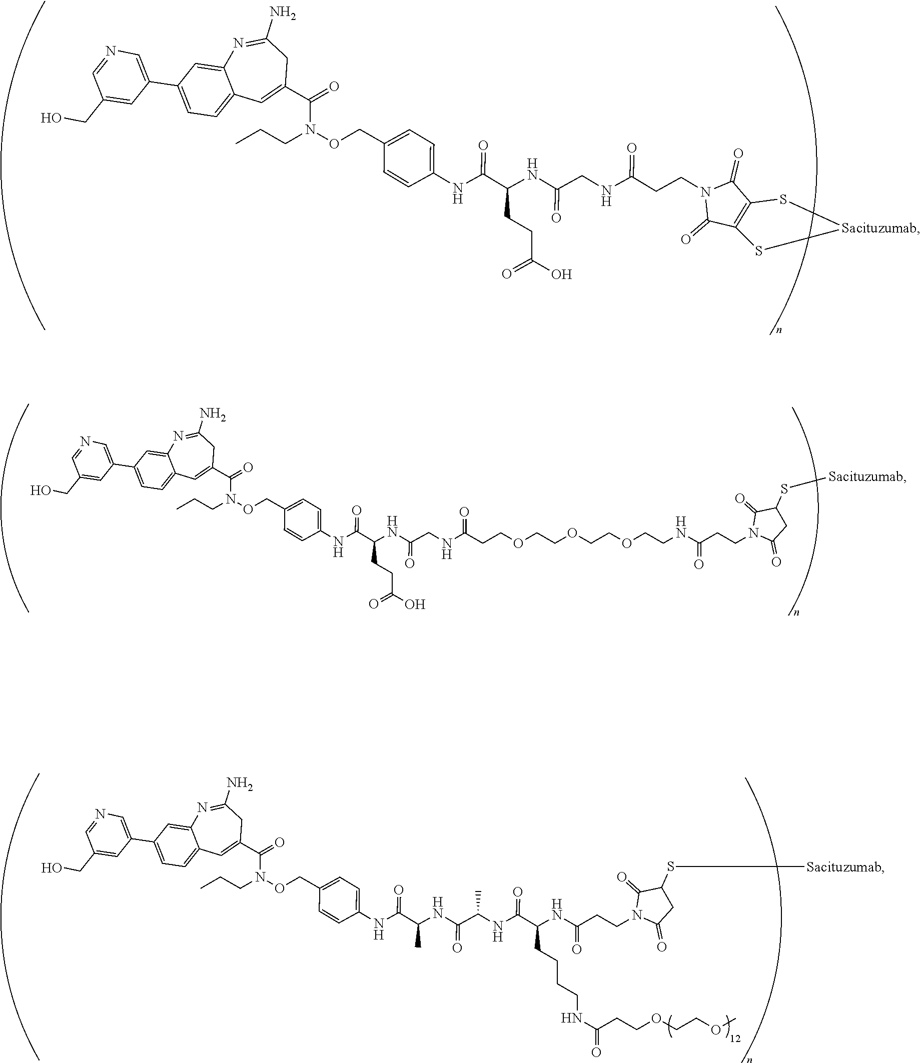 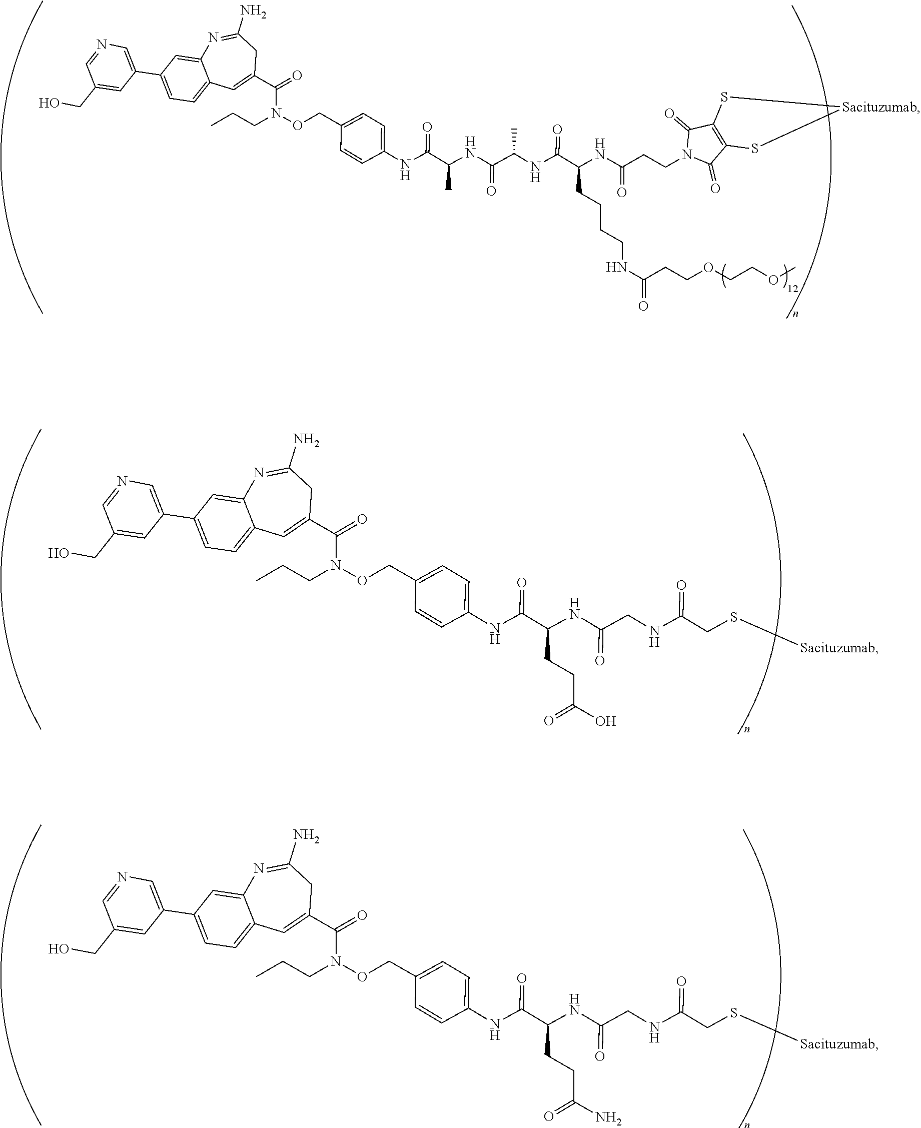 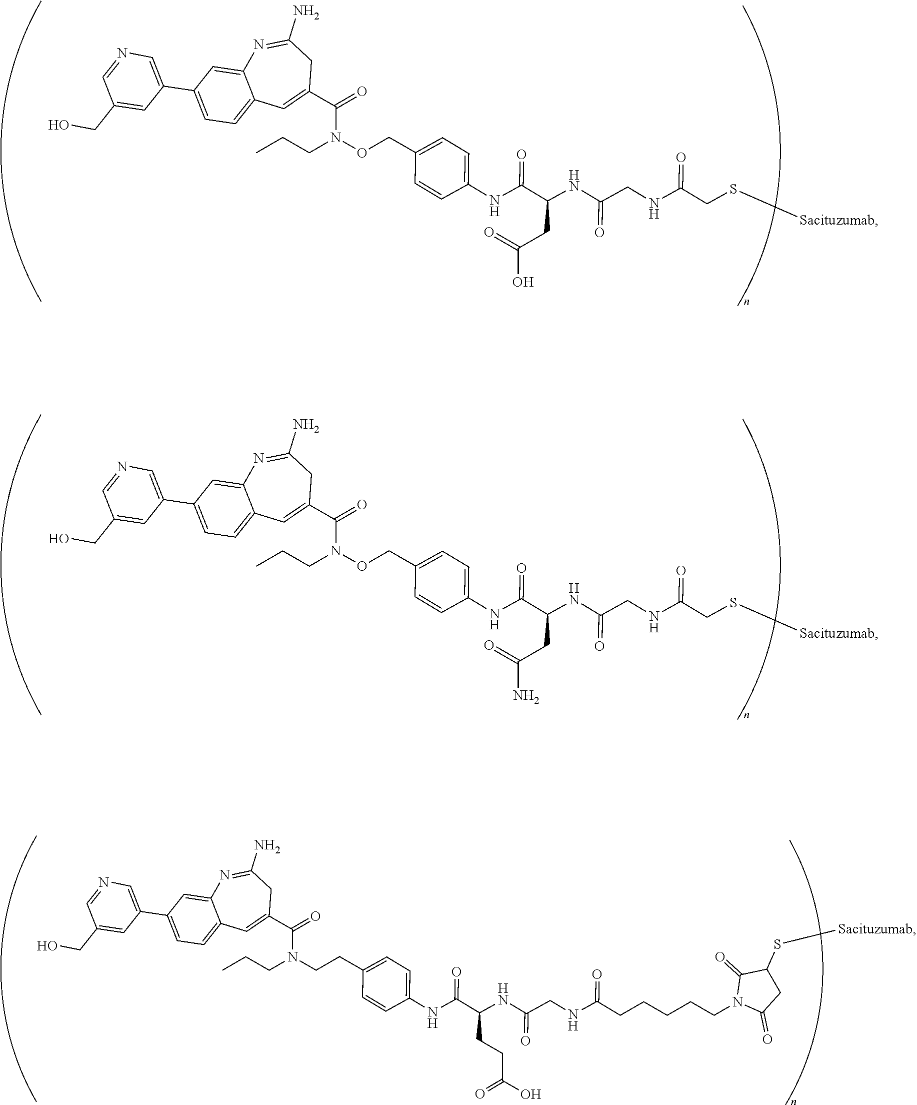 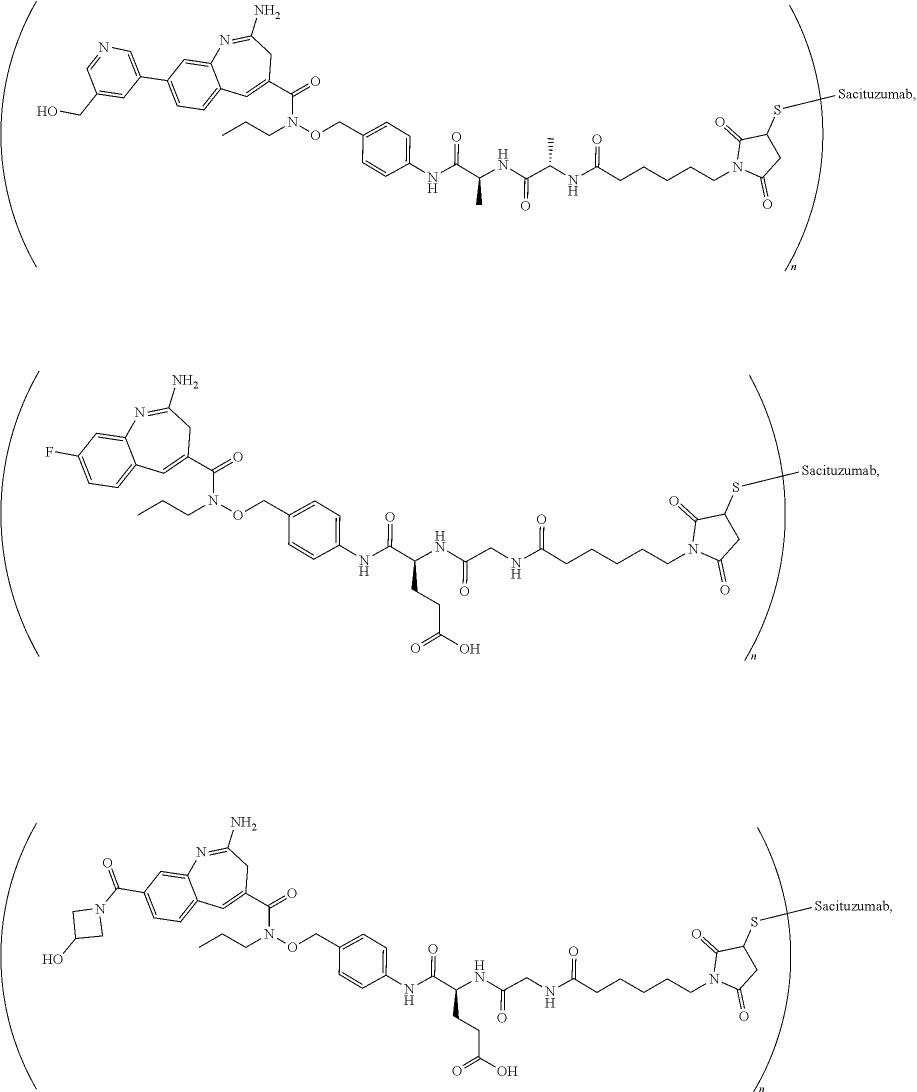 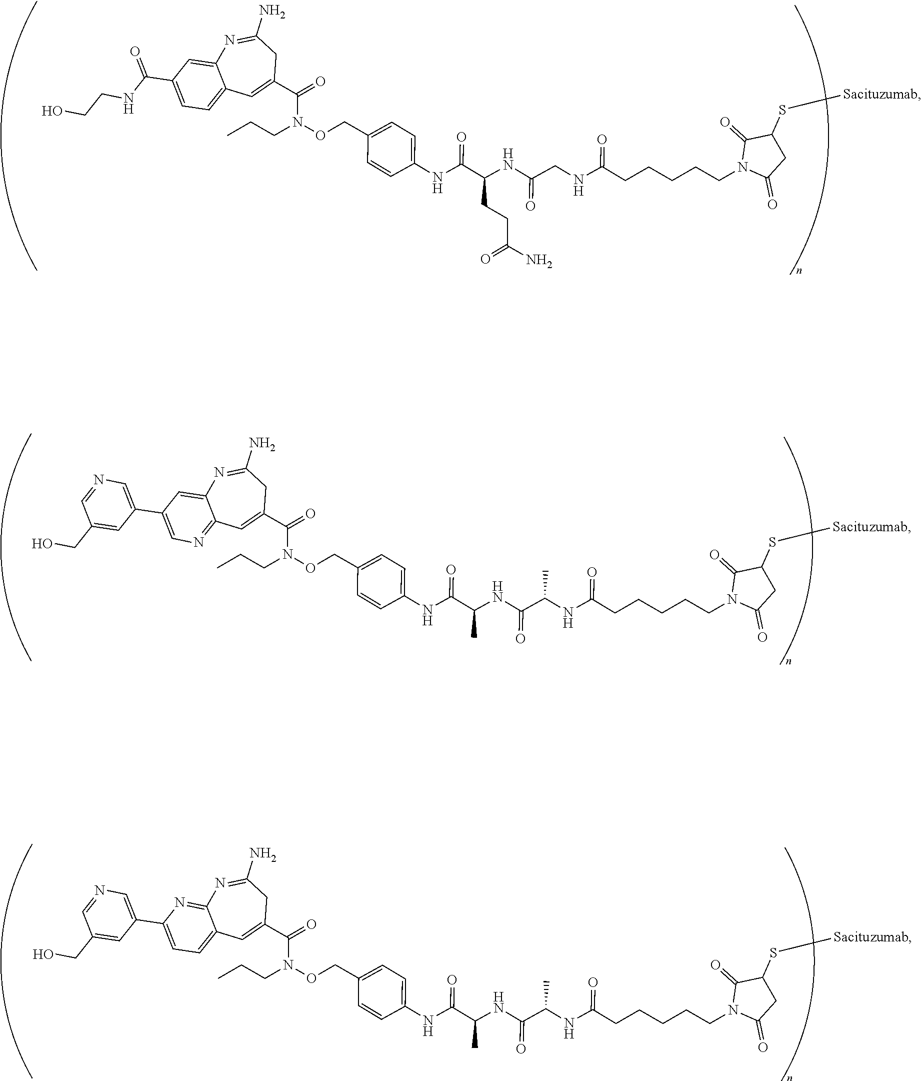 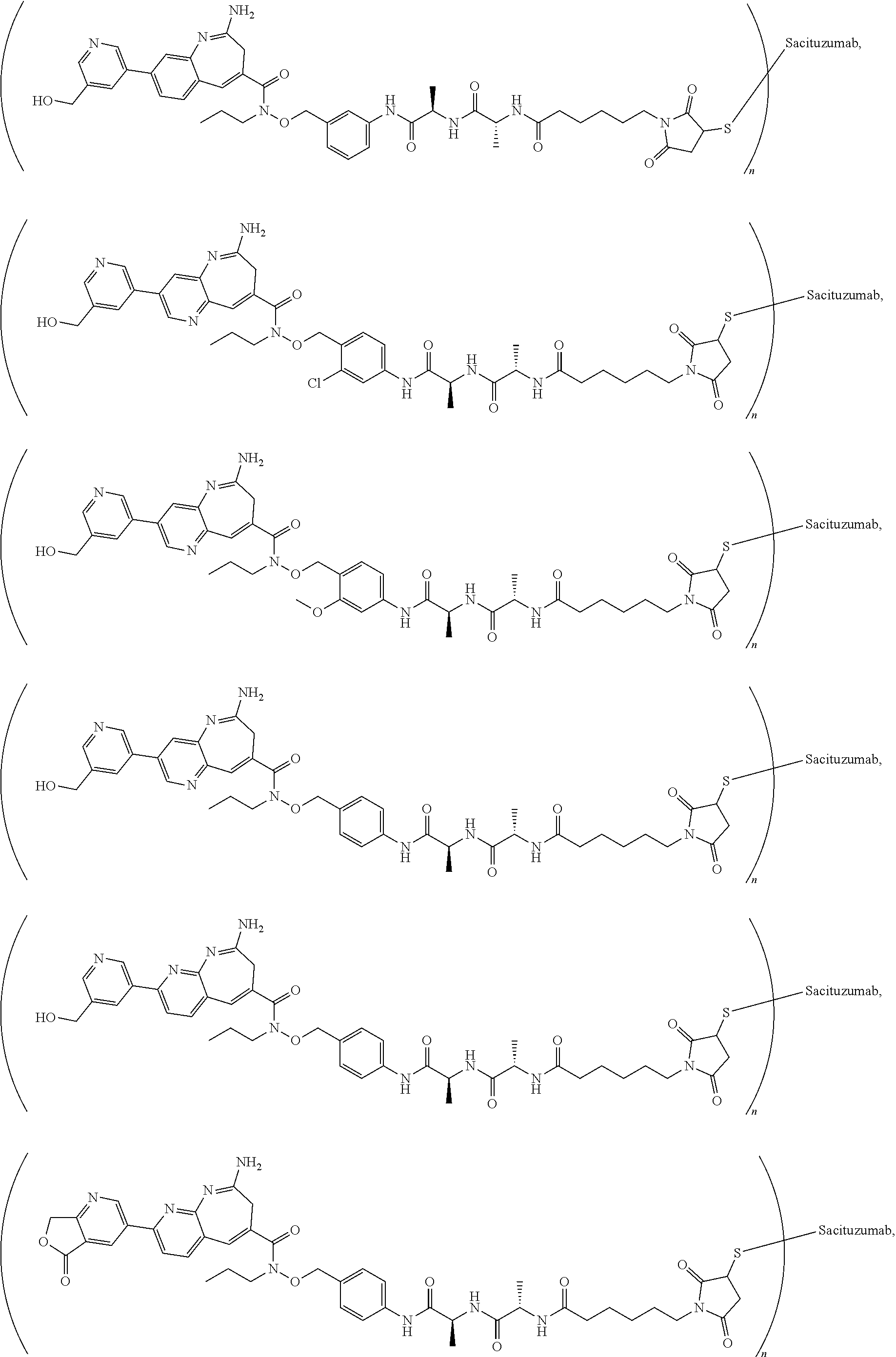 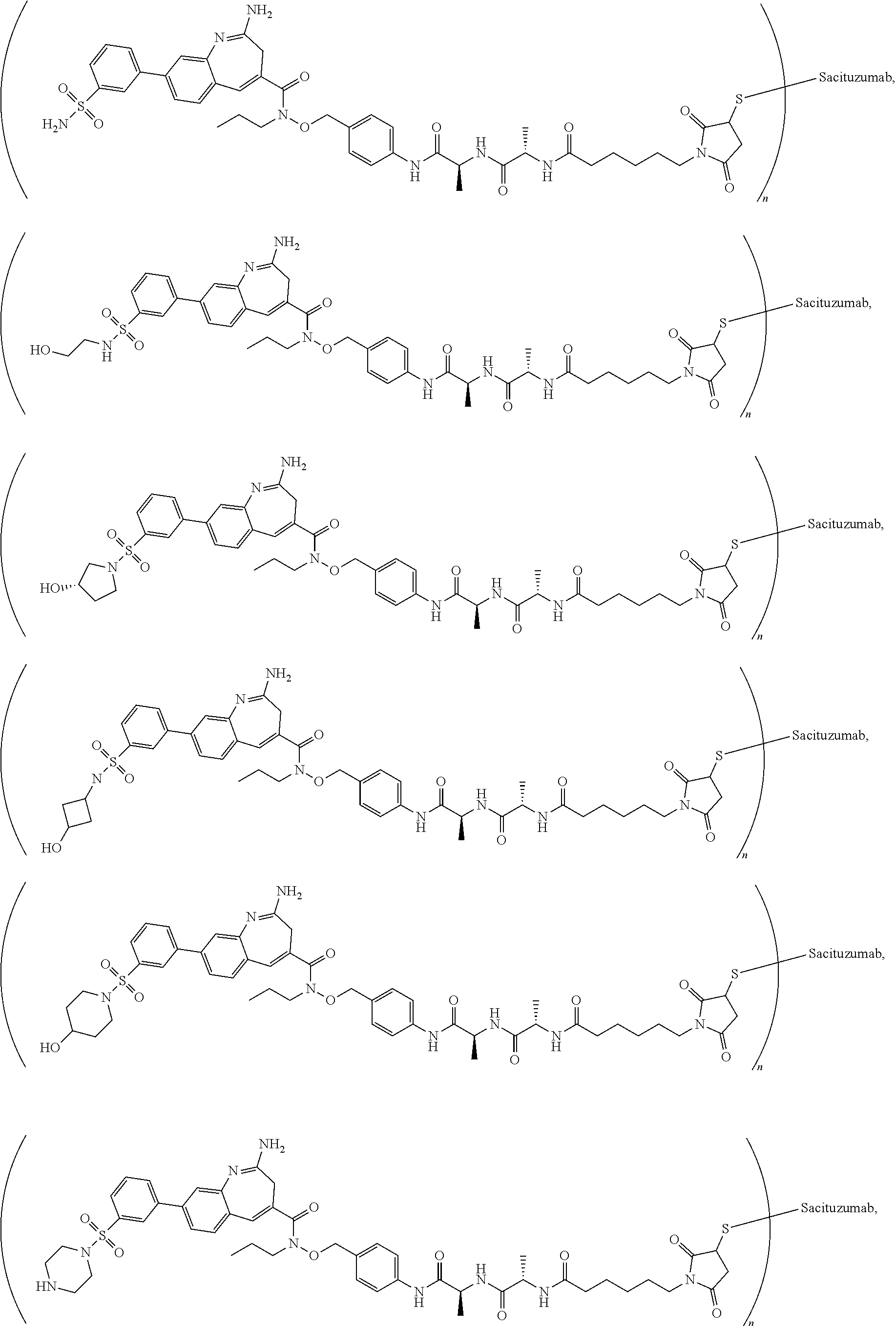 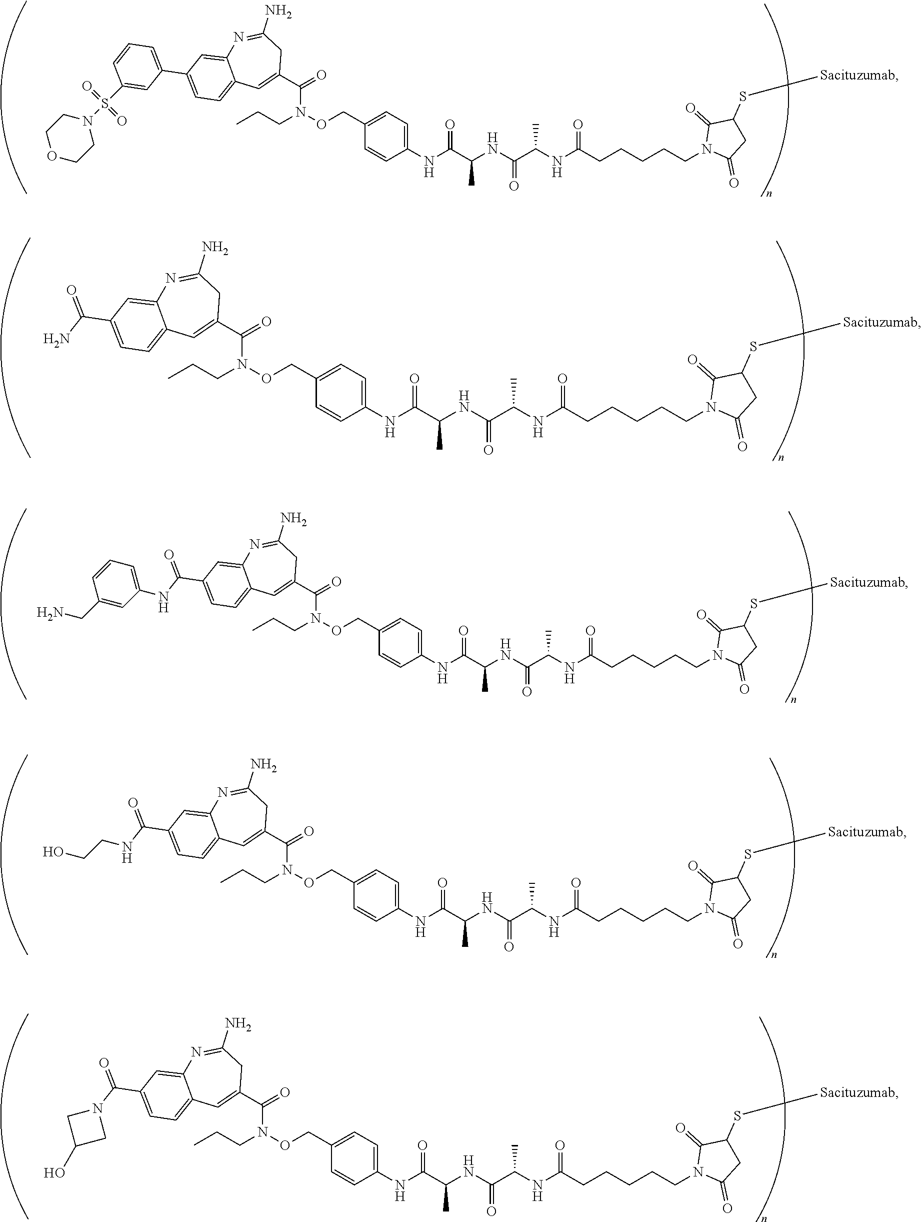 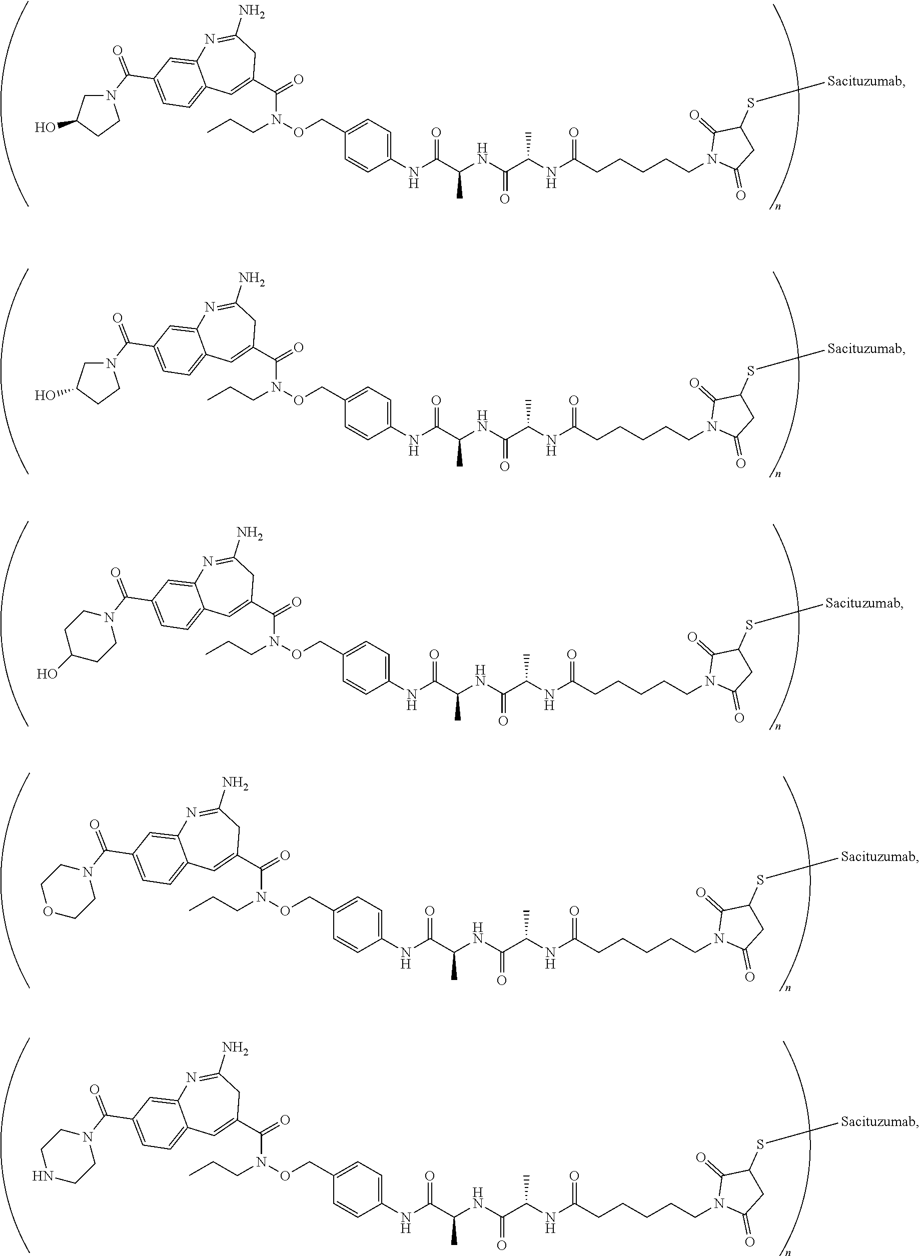 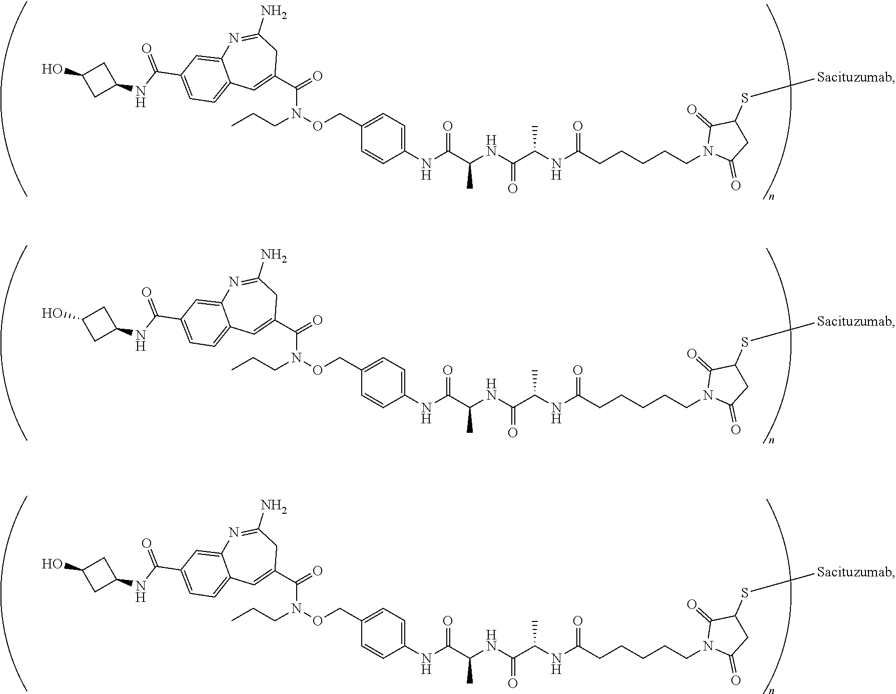 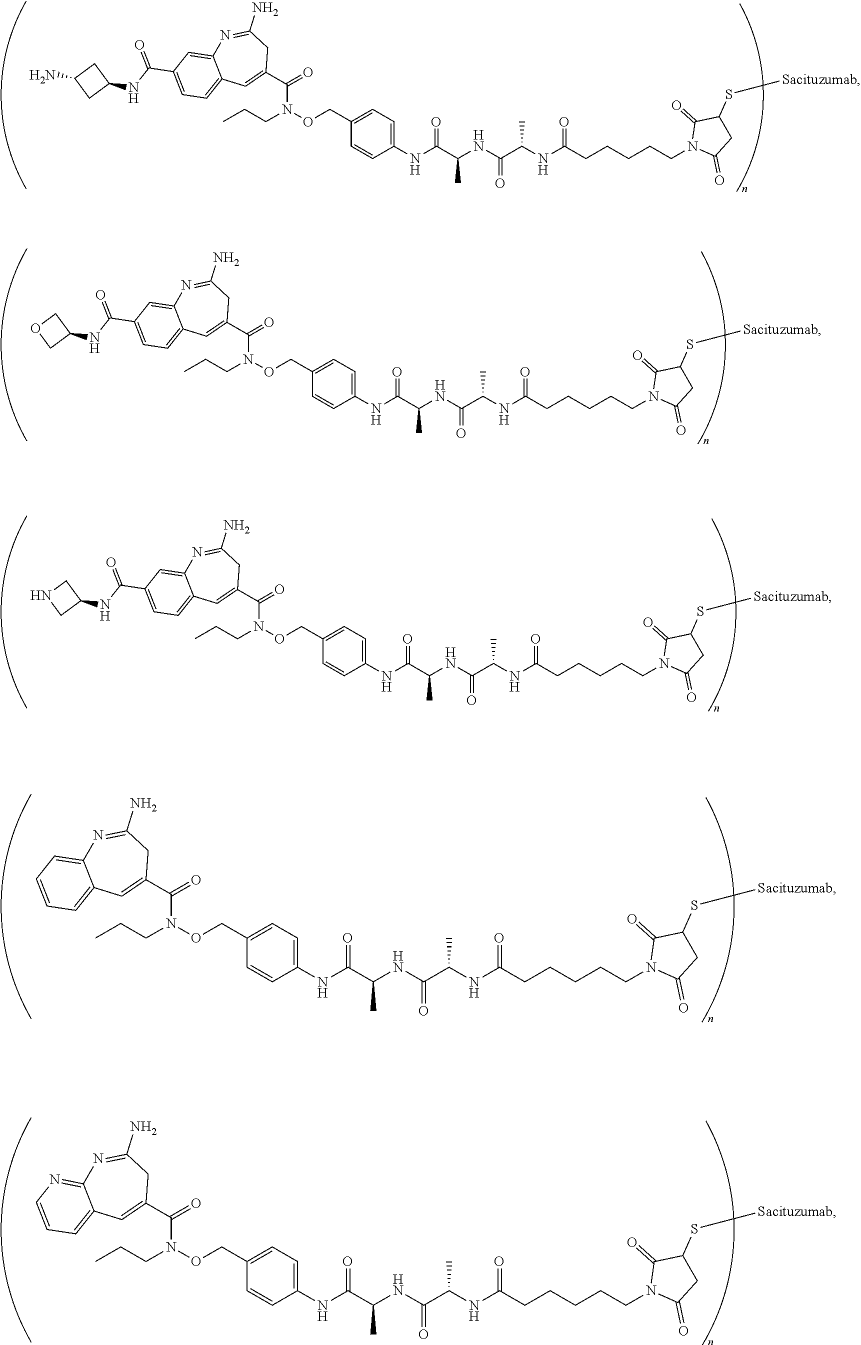 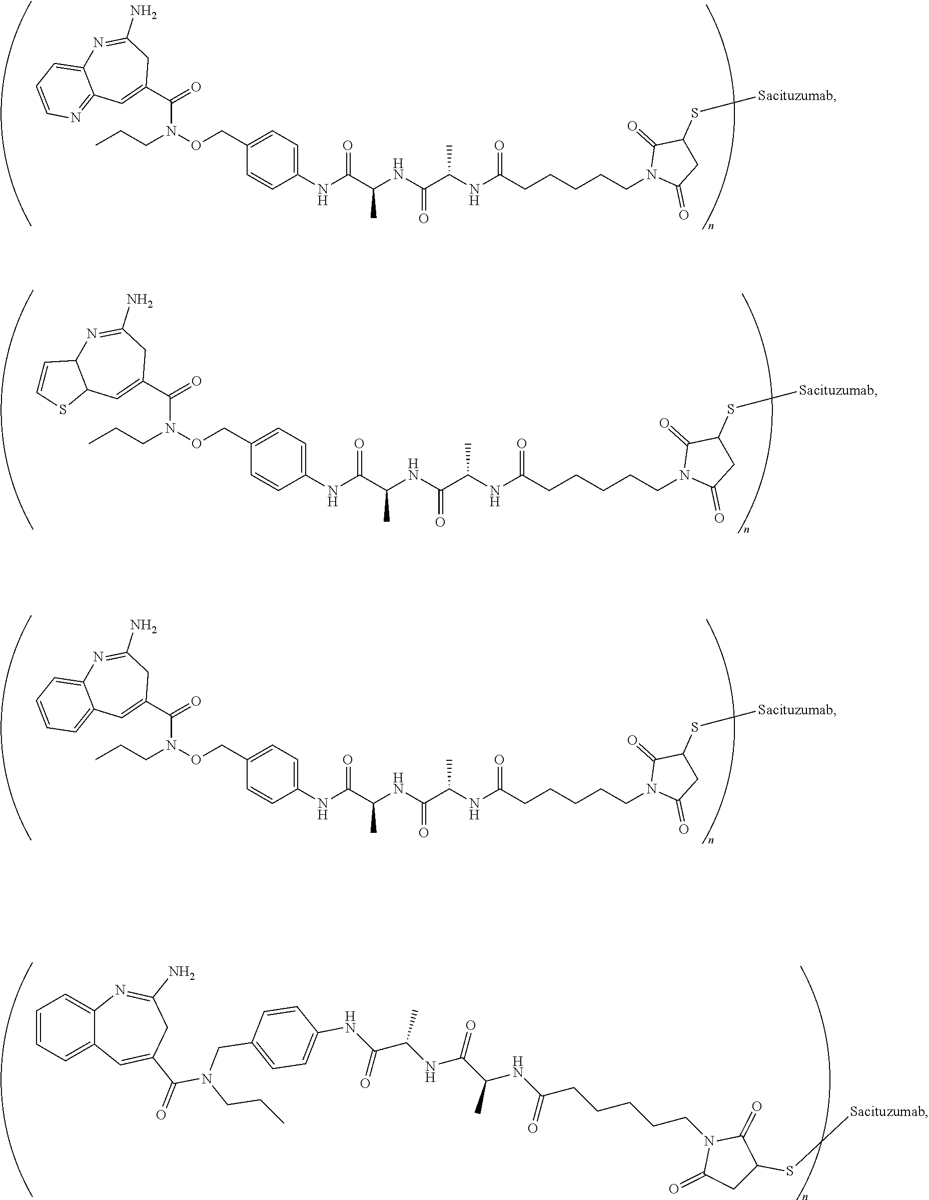 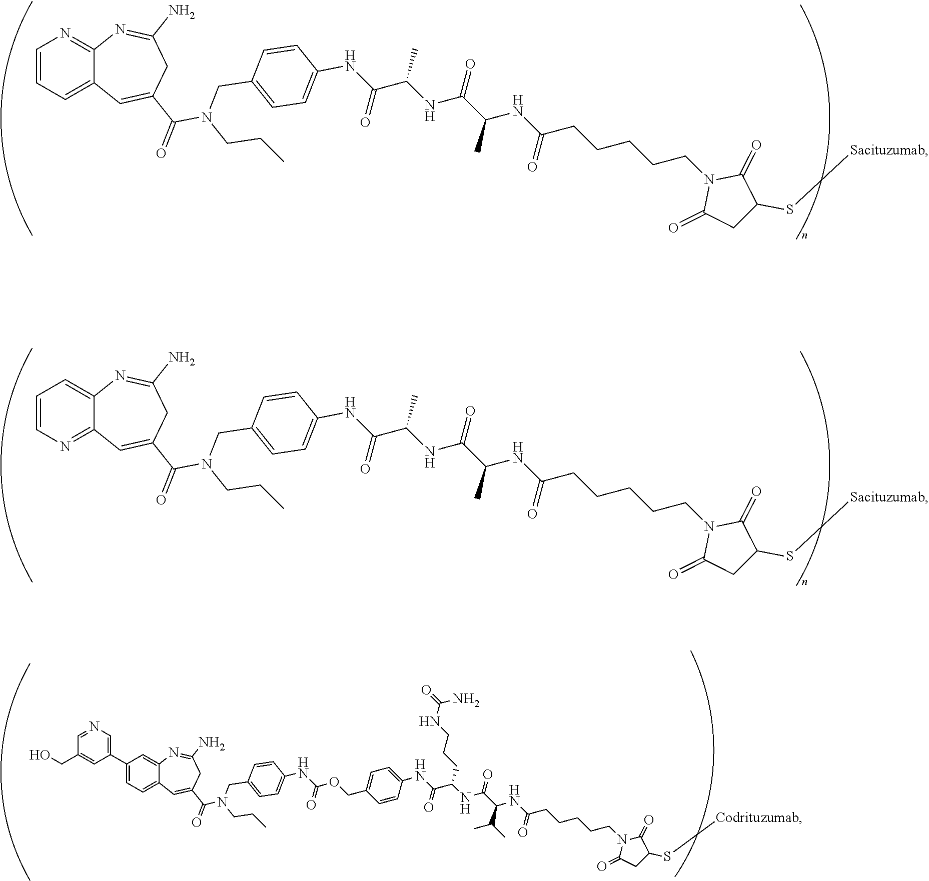 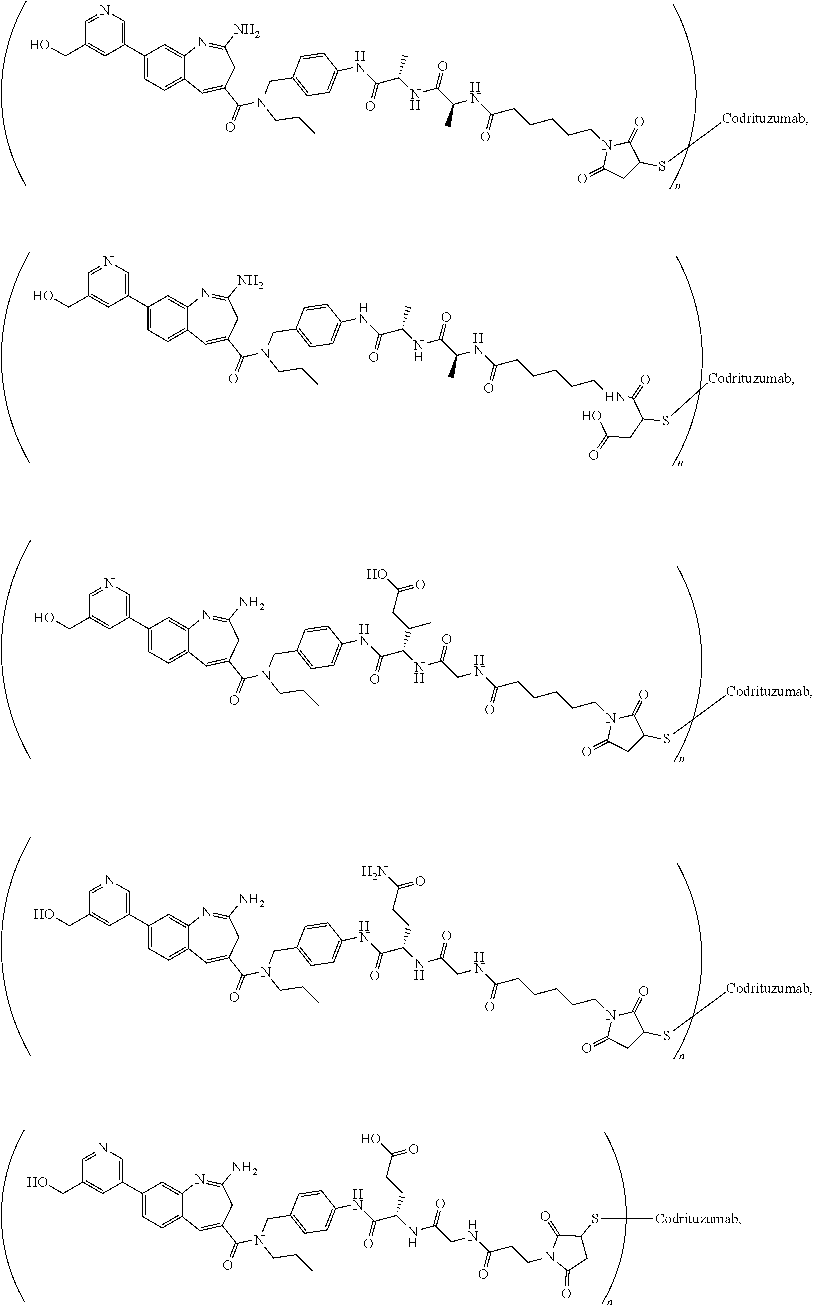 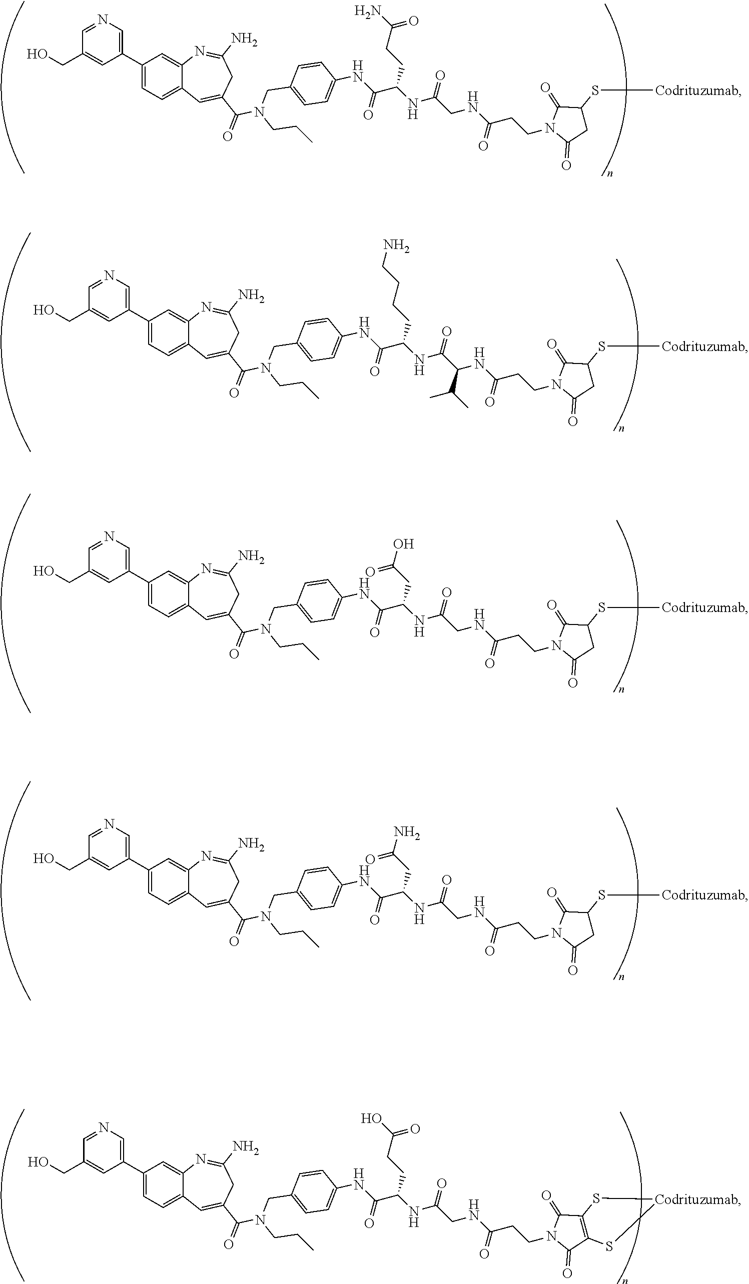 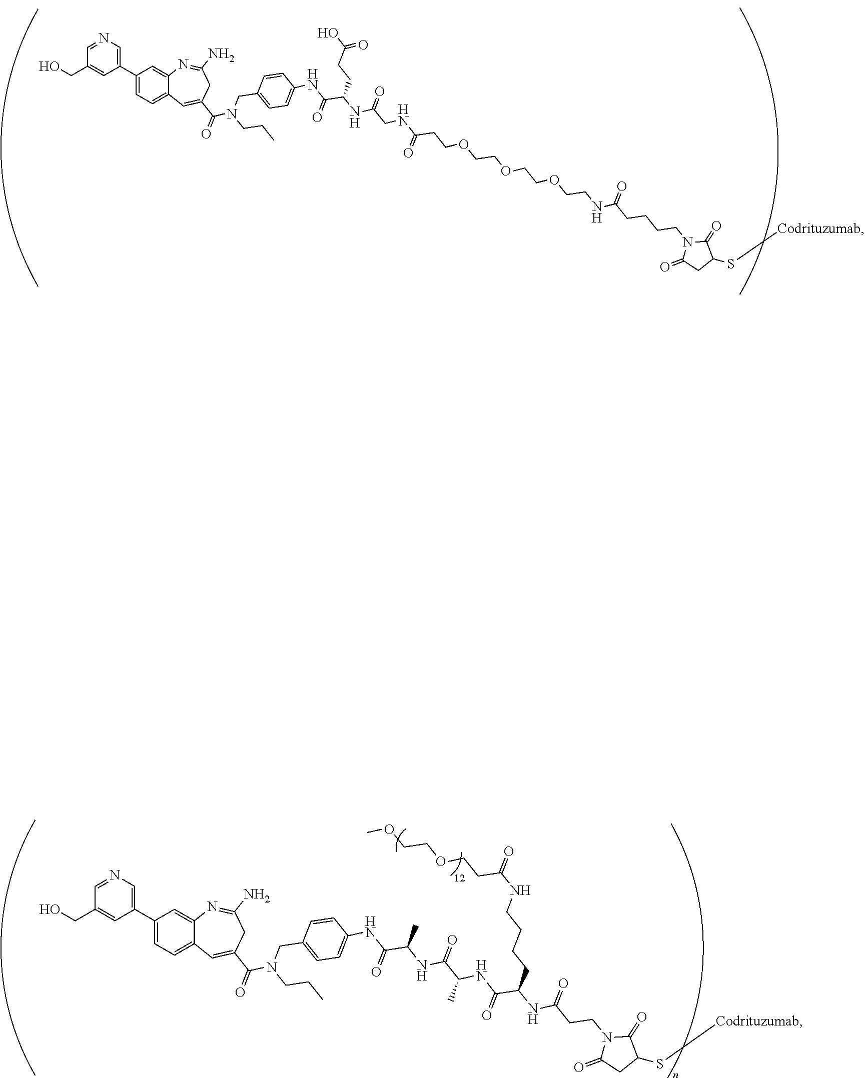 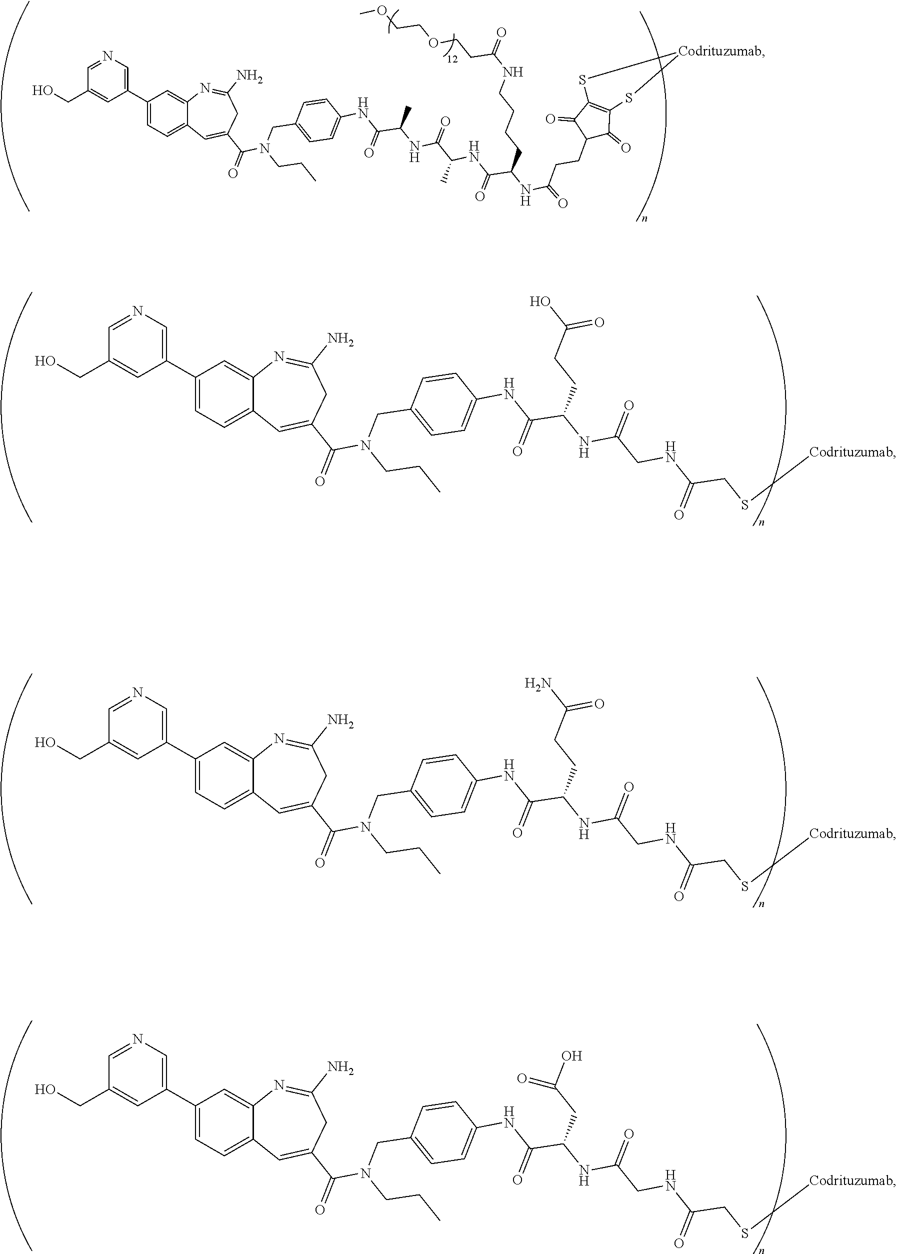 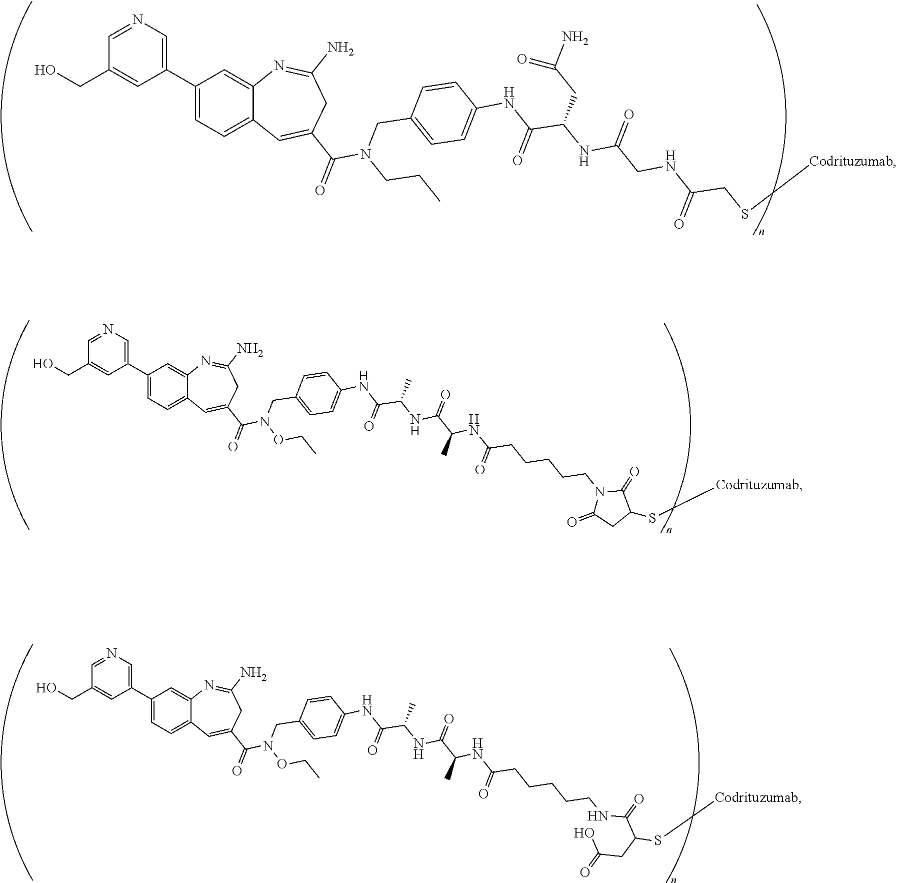 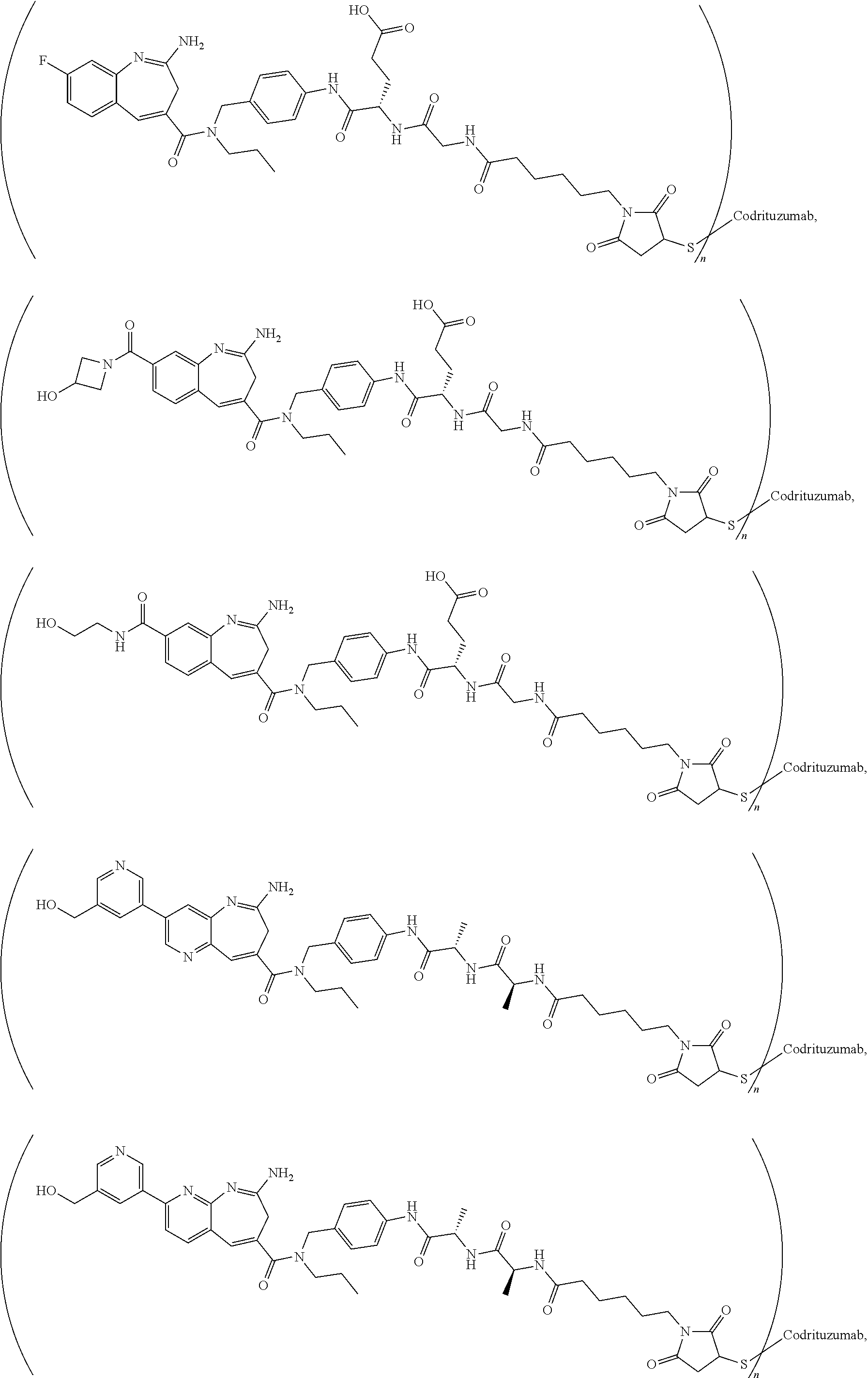 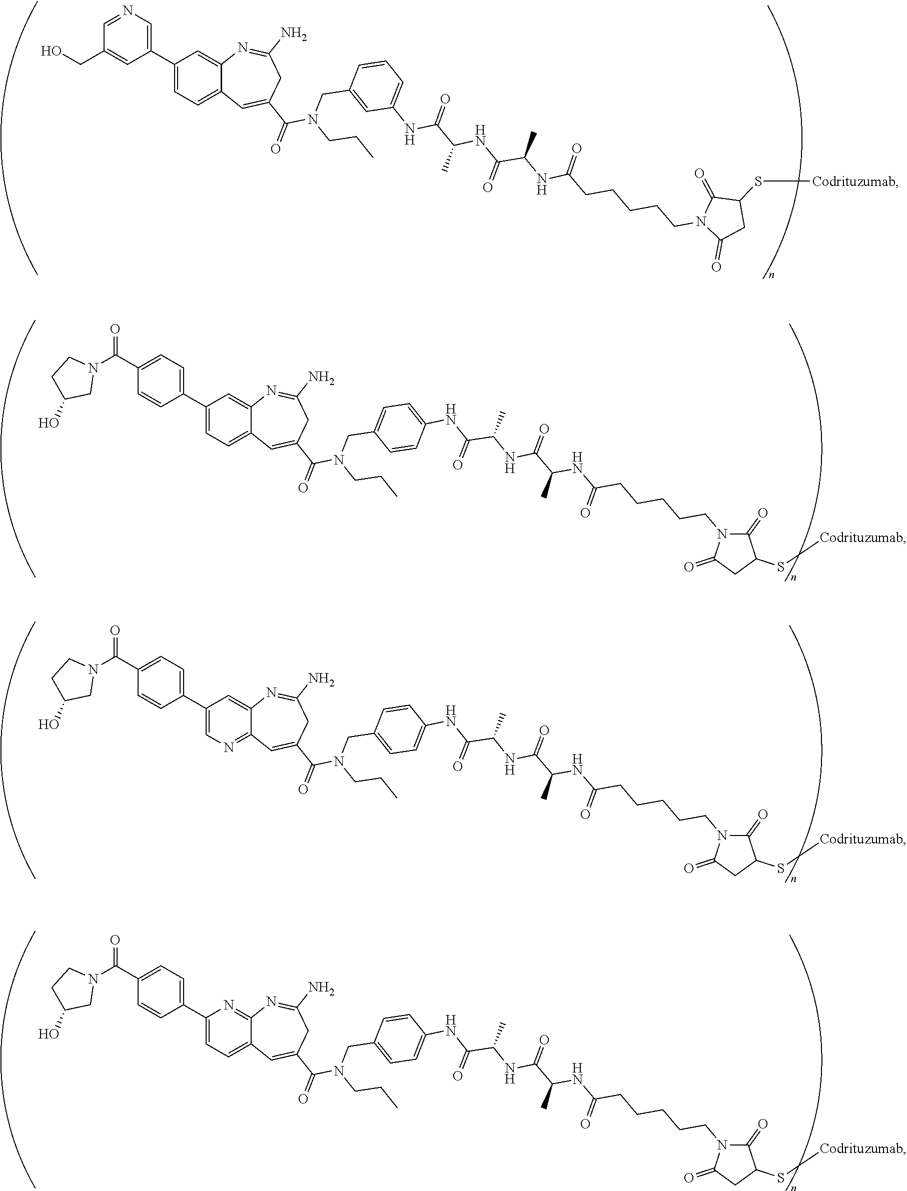 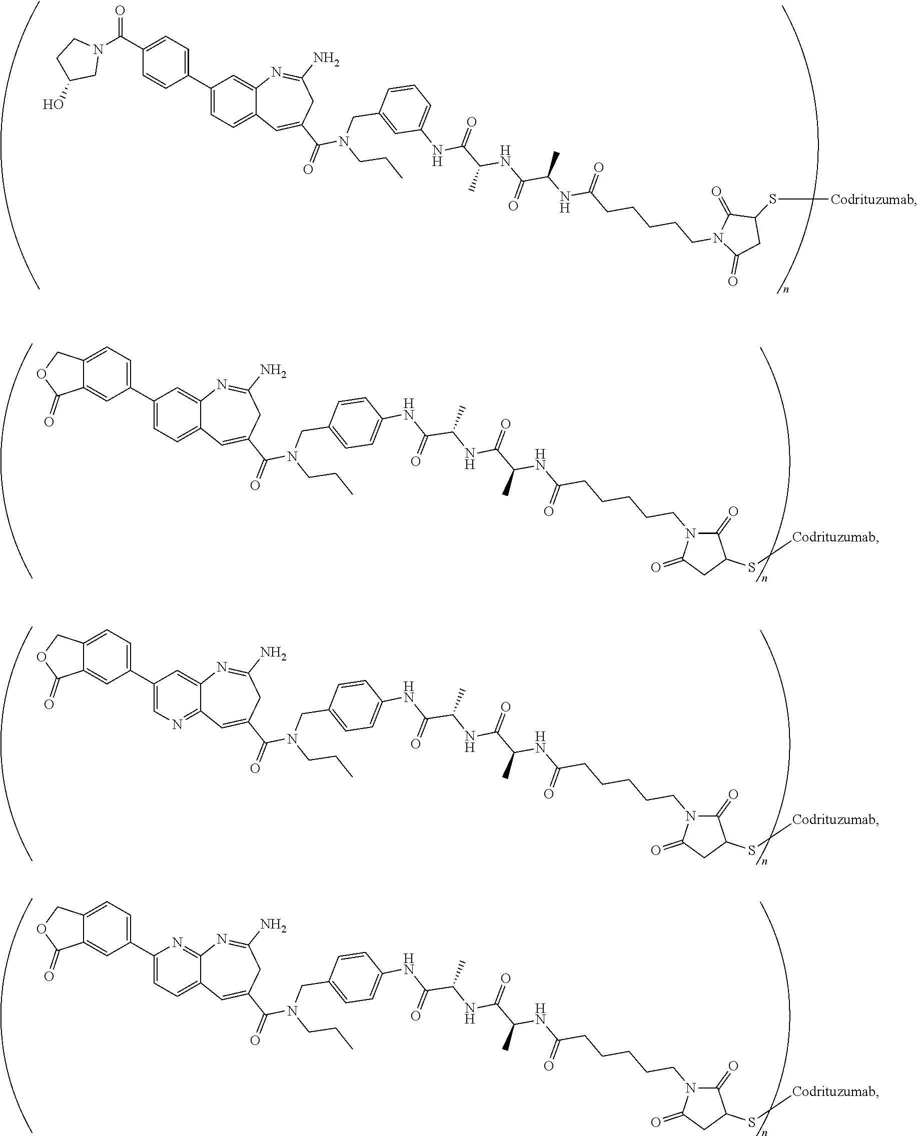 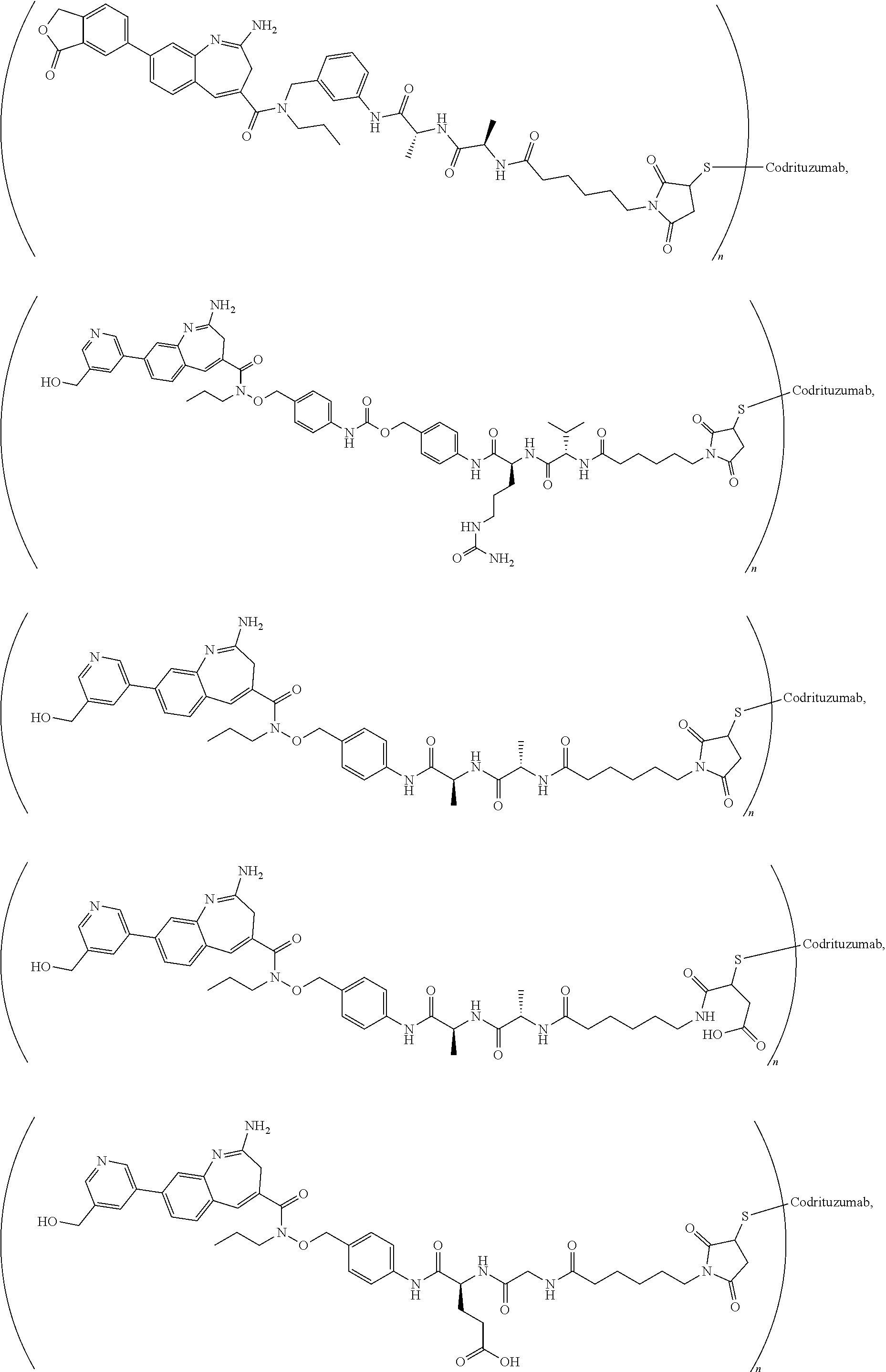 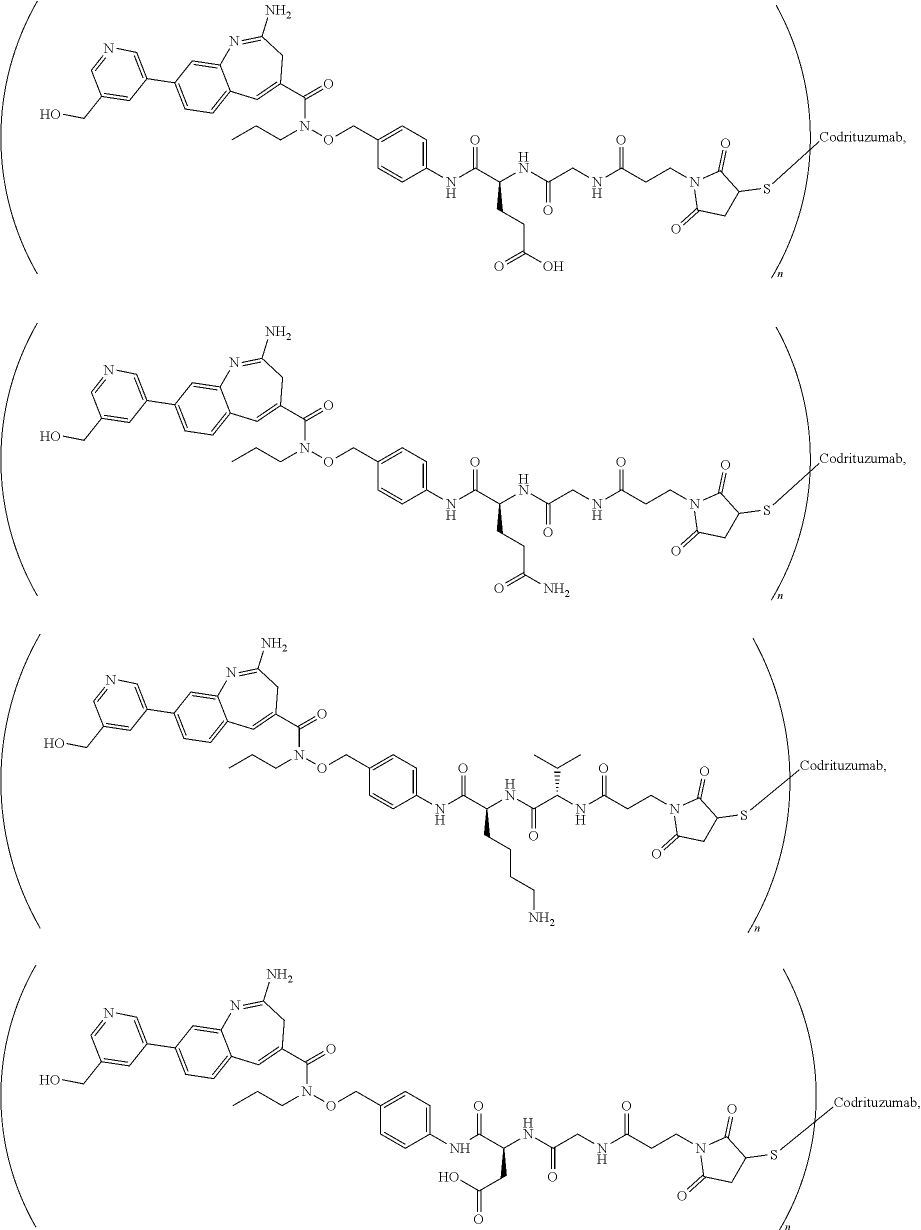 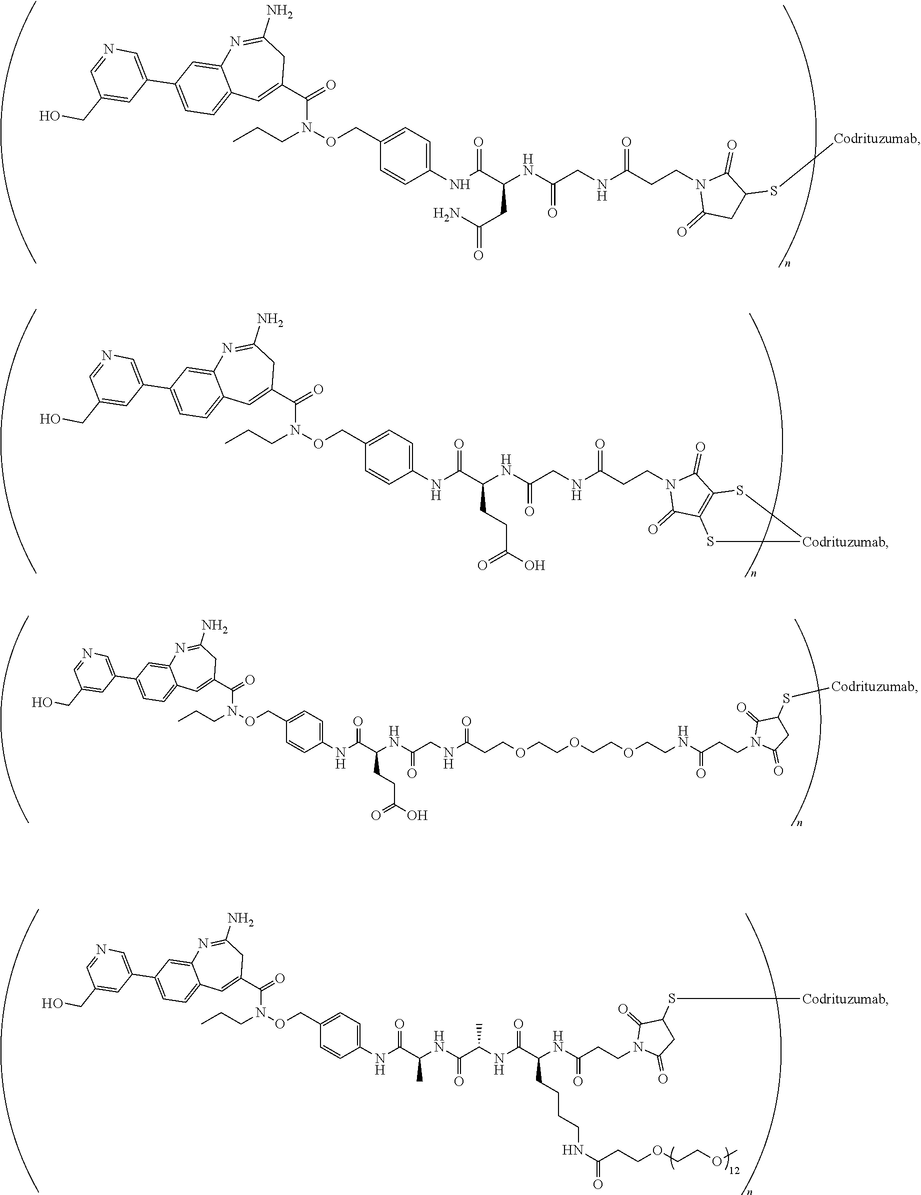 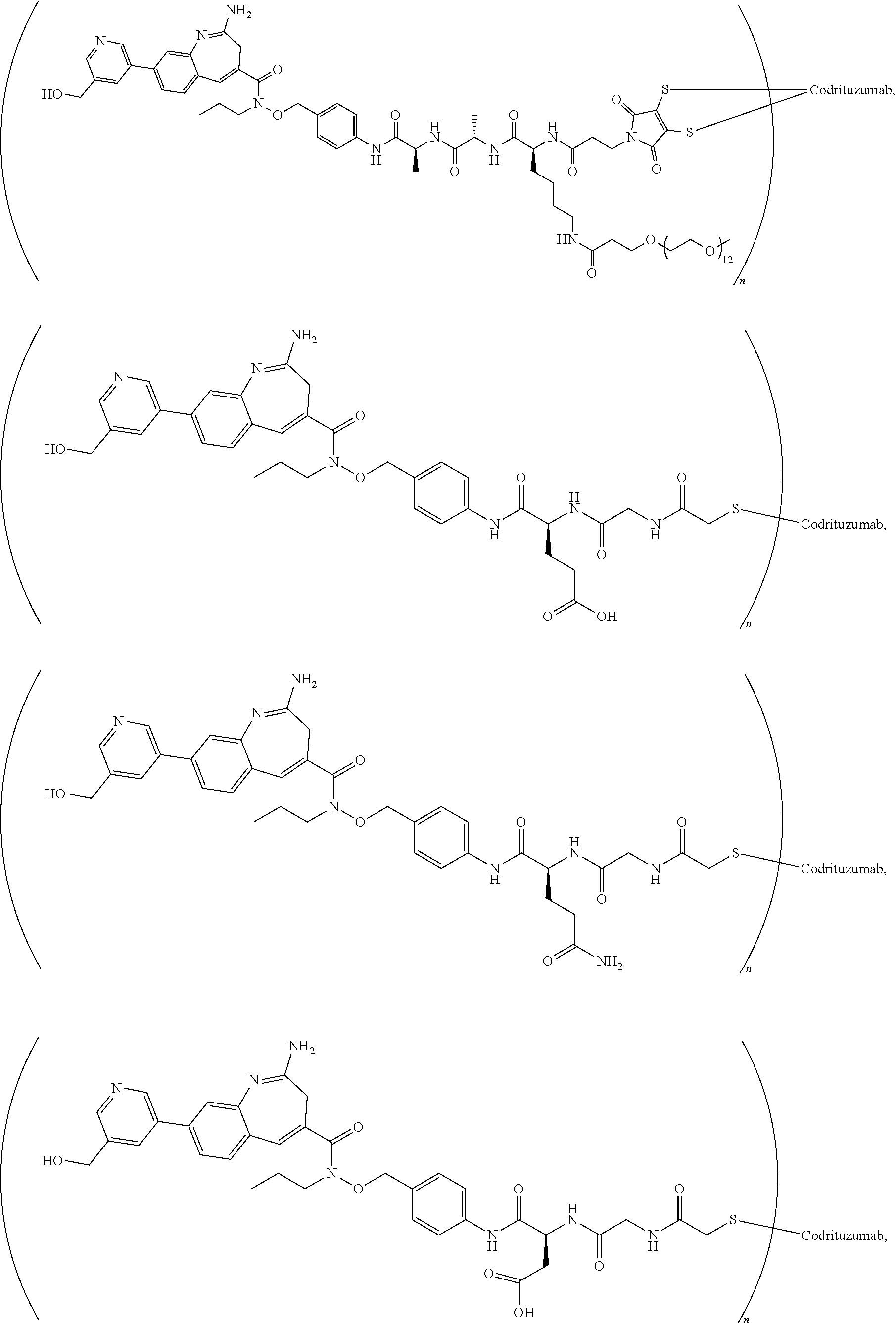 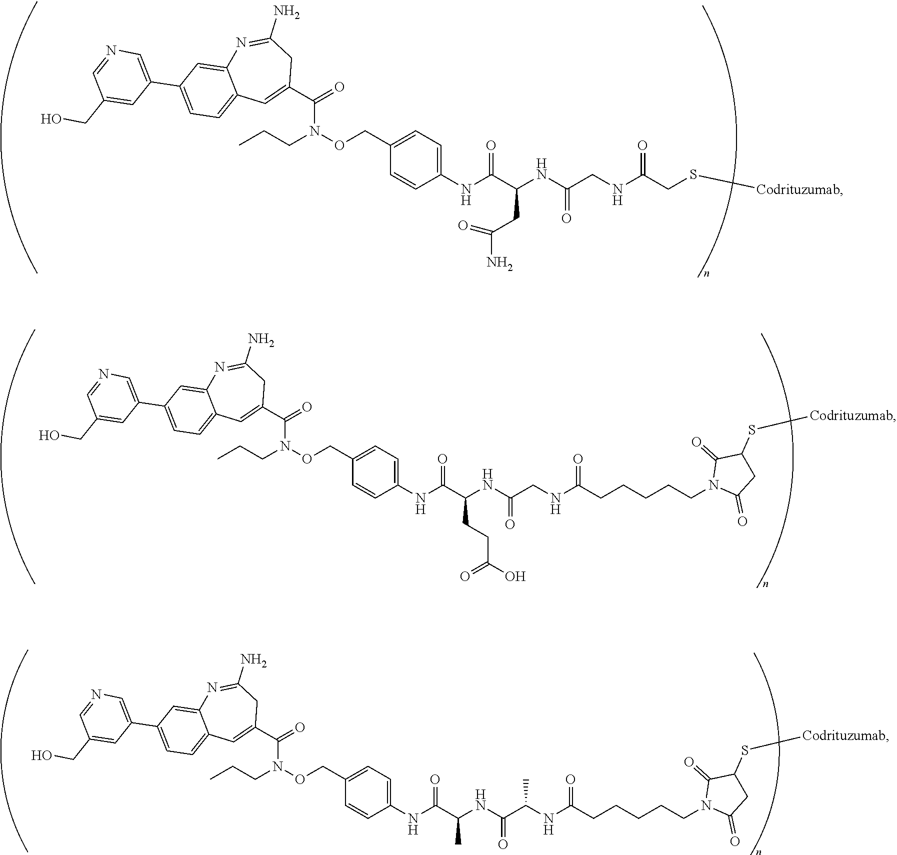 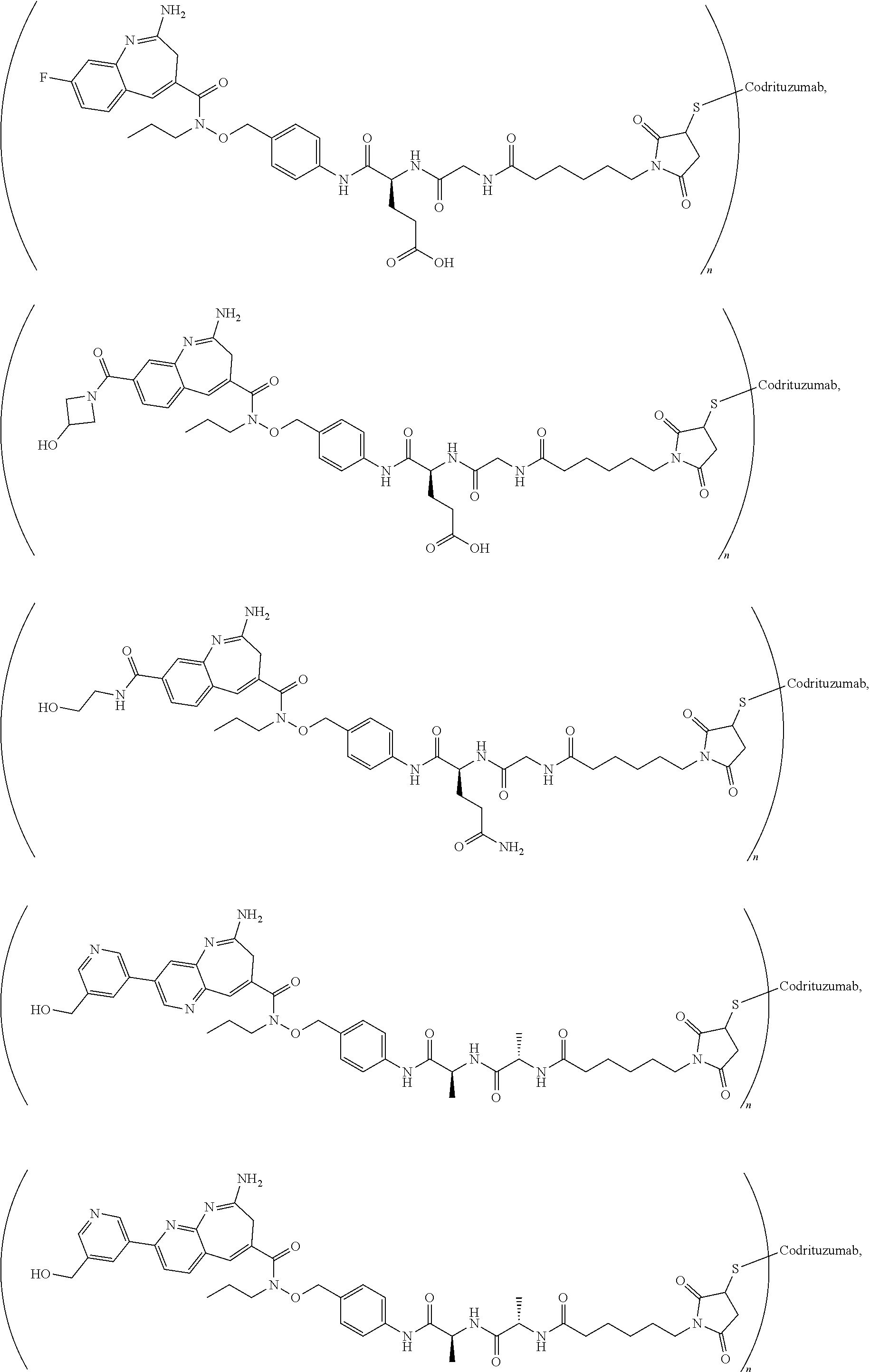 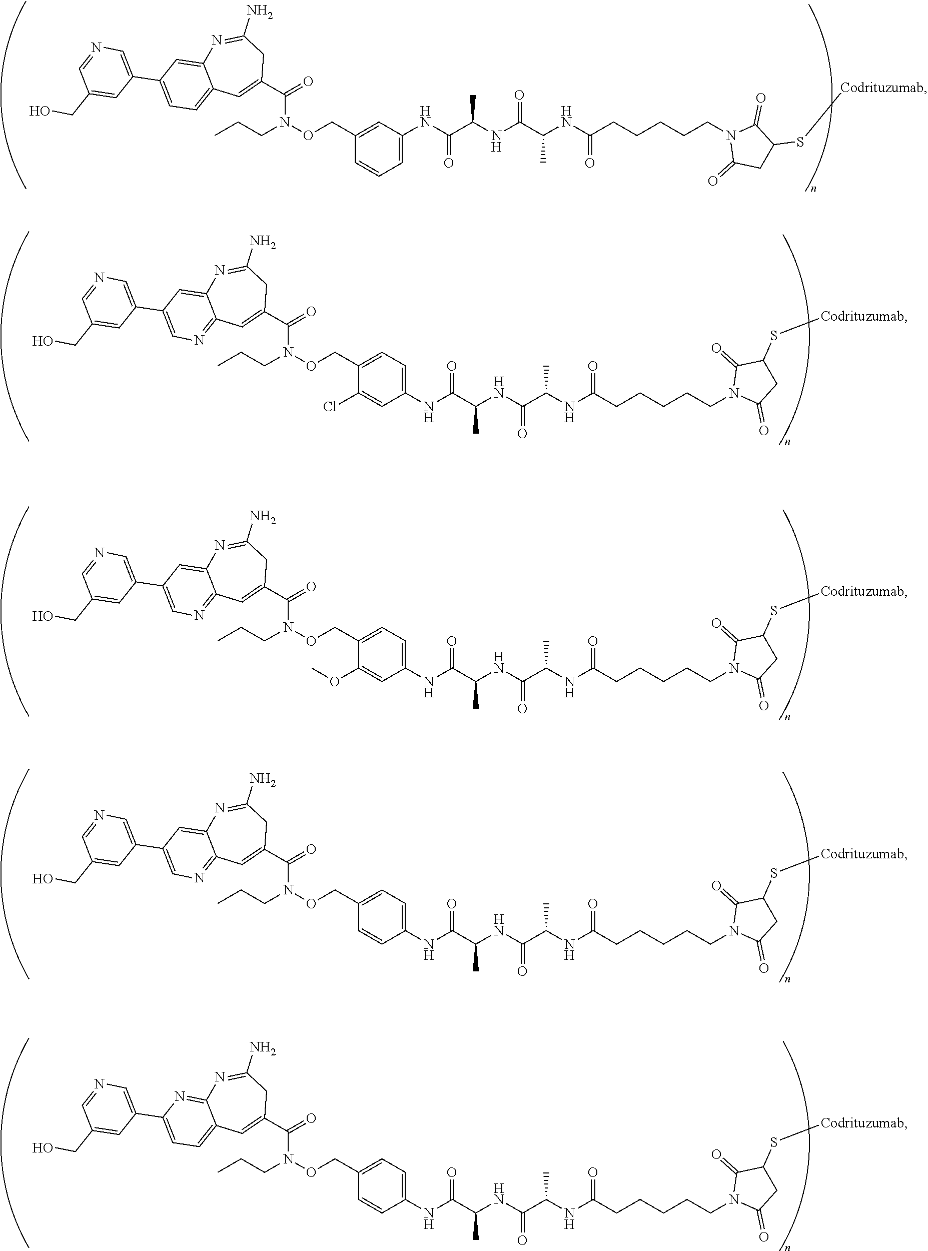 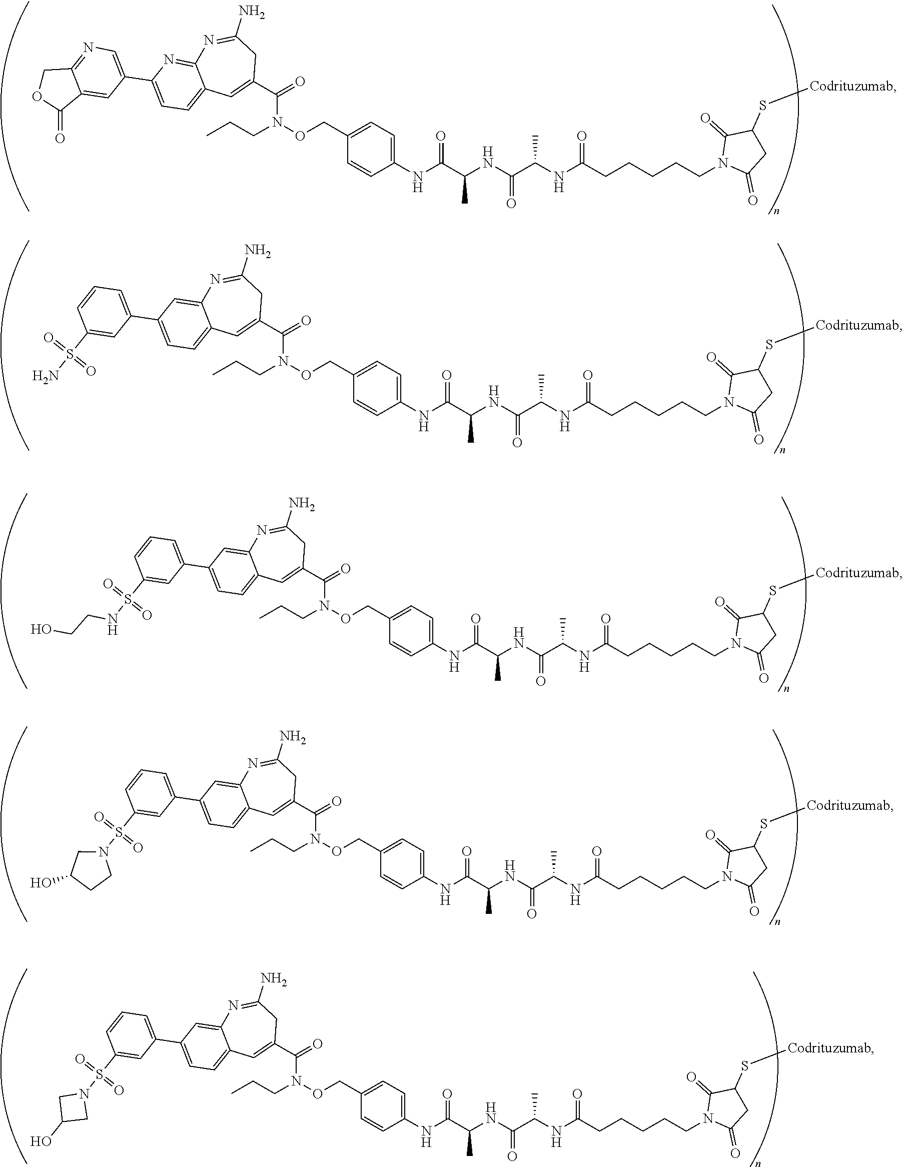 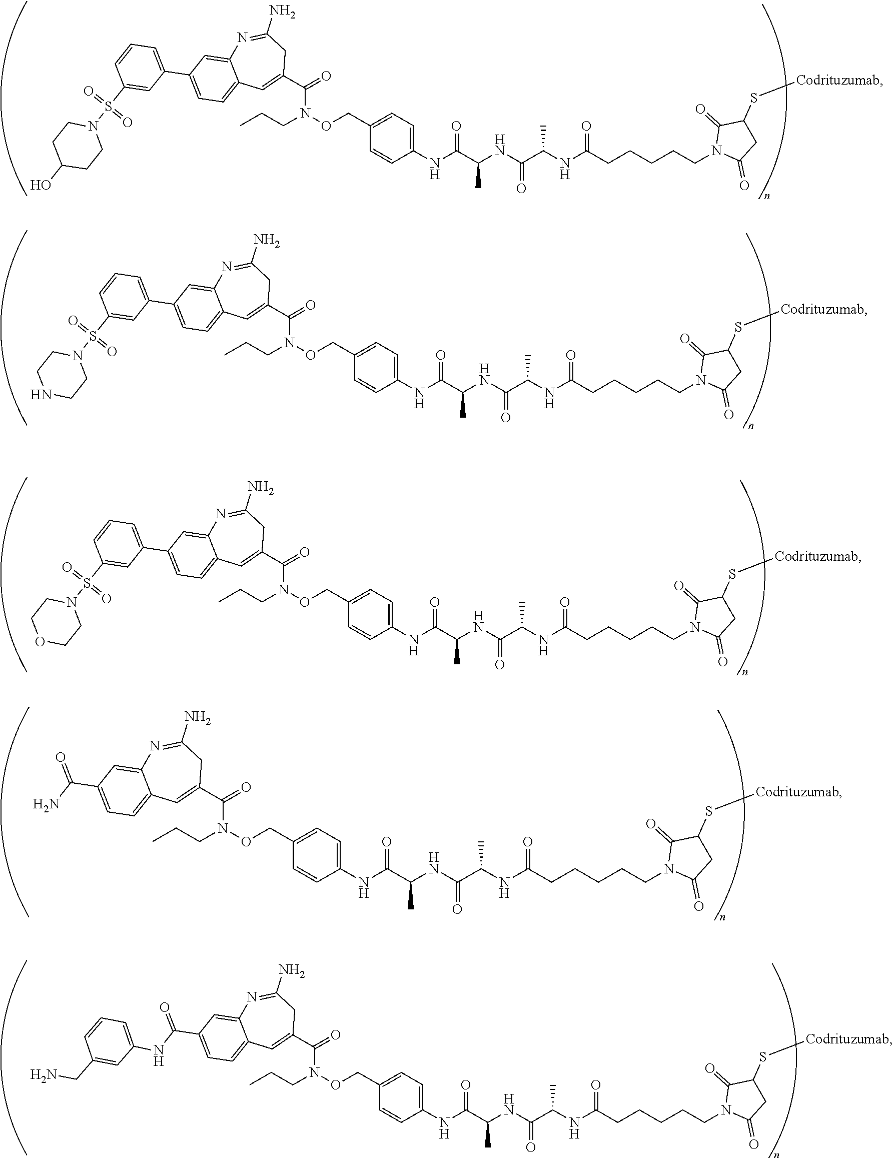 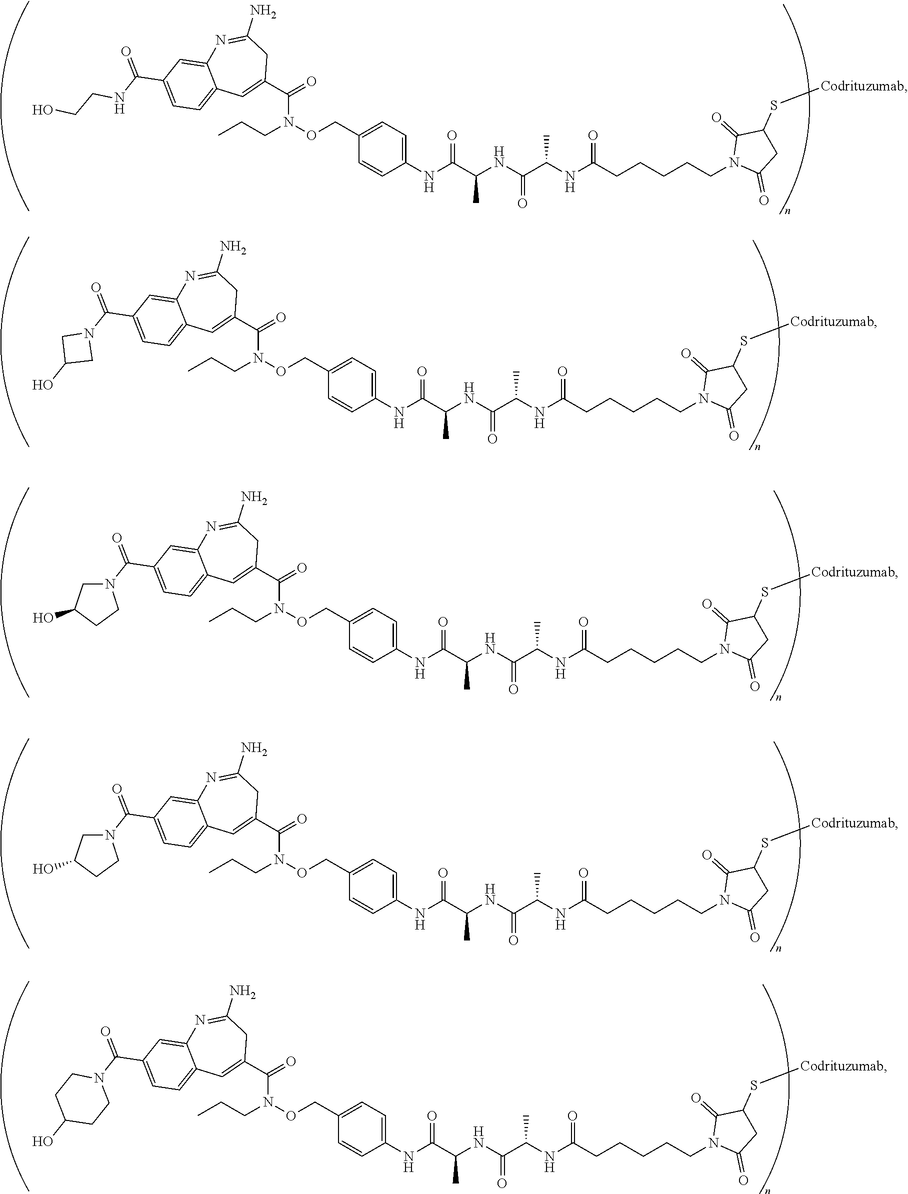 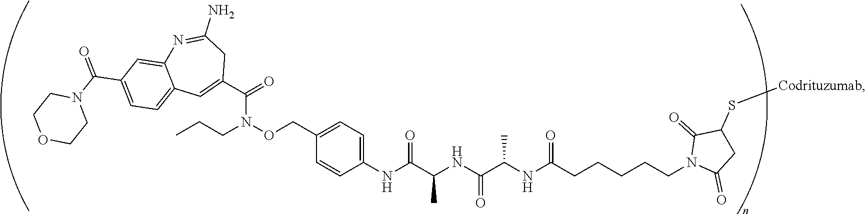 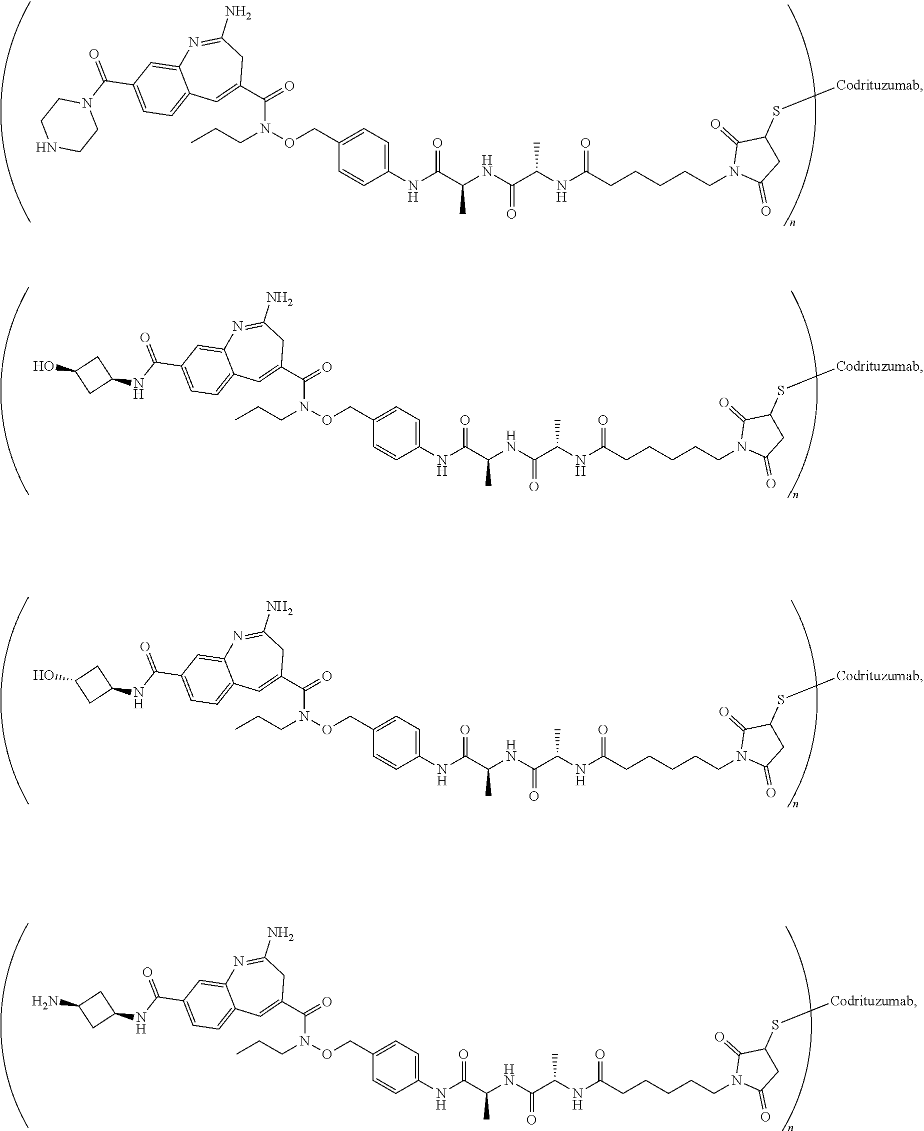 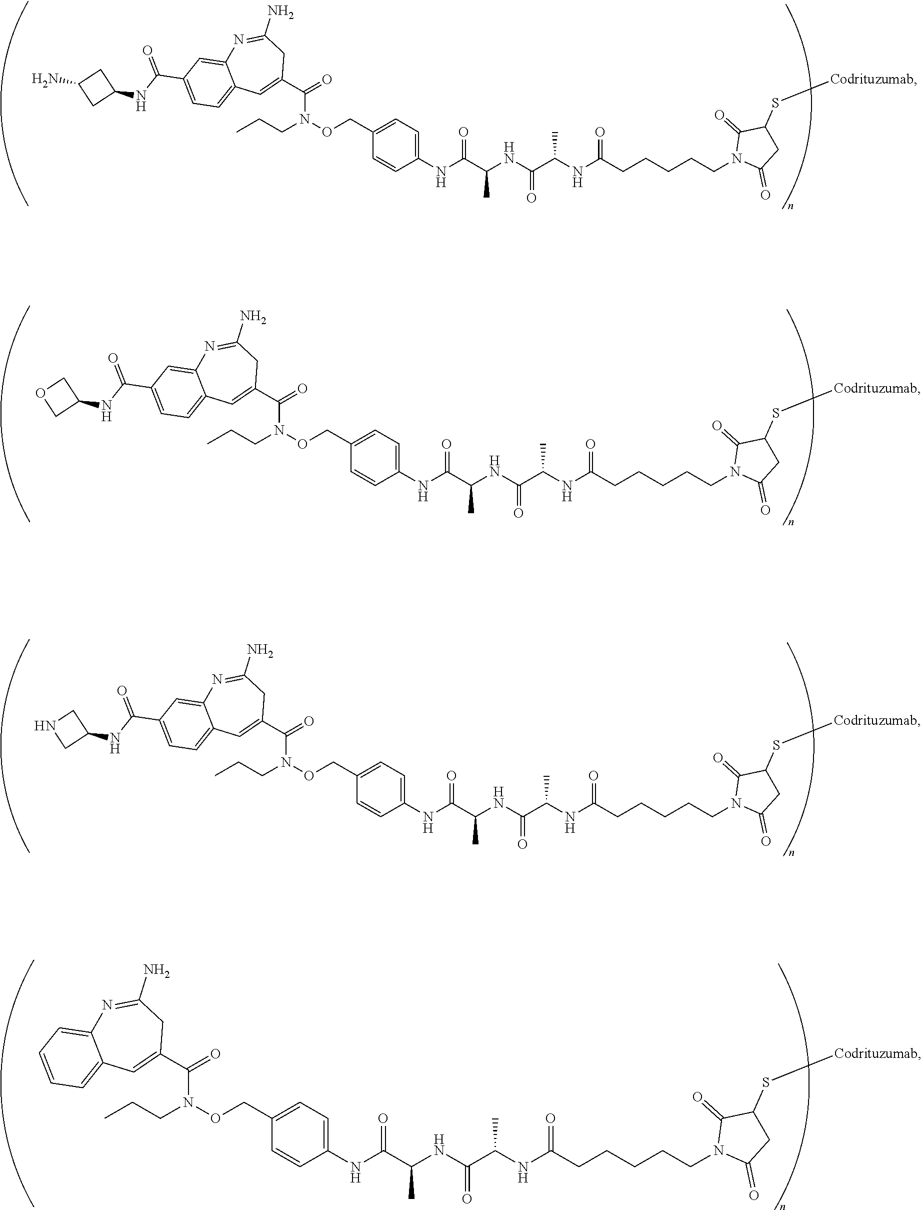 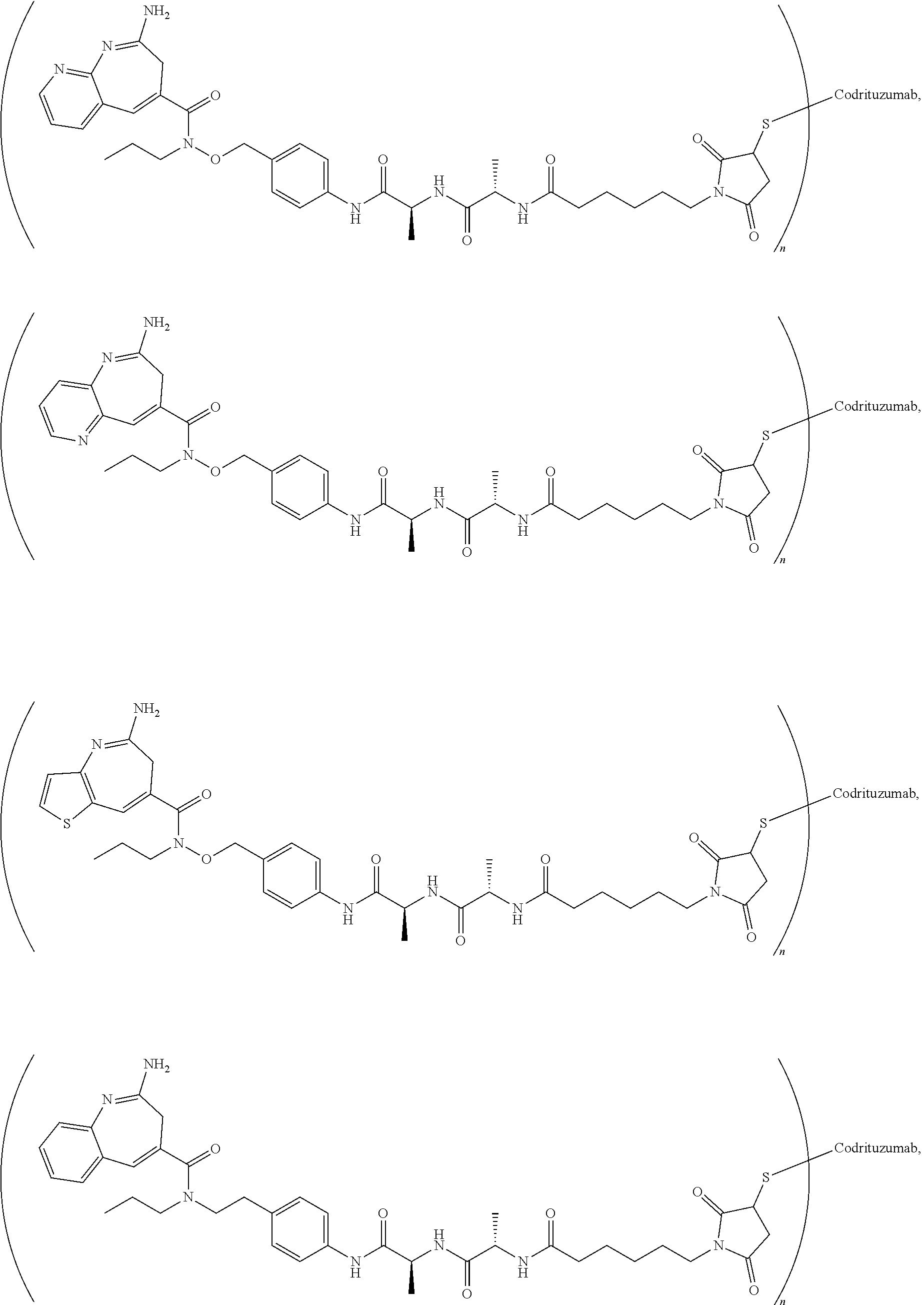 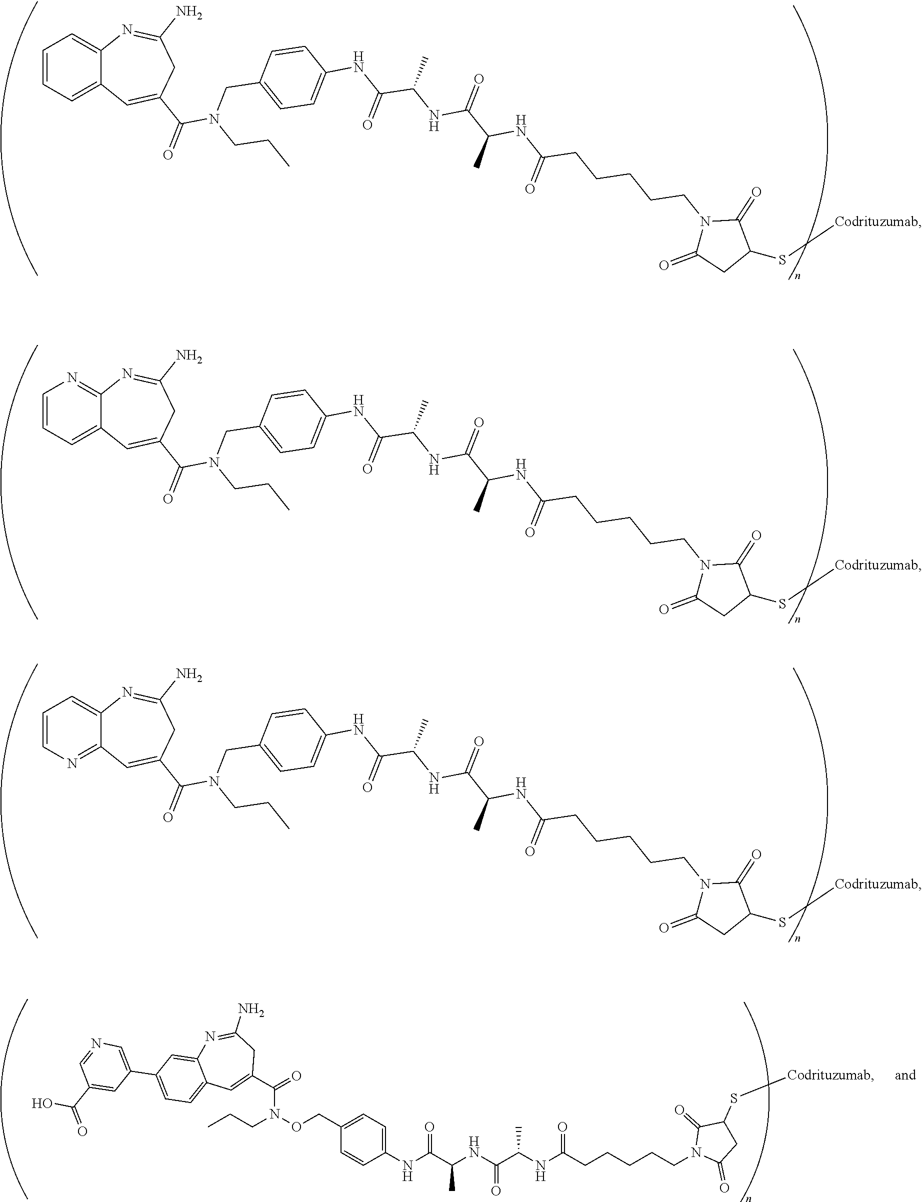 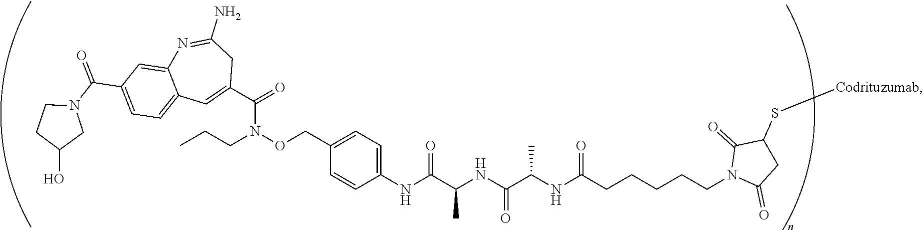
wherein
n is an integer or a decimal from 1 to 10.

37. A compound or a conjugate thereof, or an isomer thereof, or a mixture thereof, or a pharmaceutically acceptable salt thereof, wherein the compound has a structure represented by formula (II-3A): 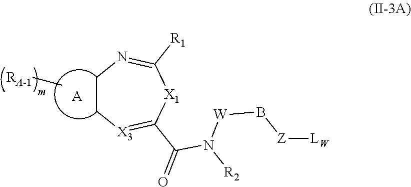
wherein
R₁ is optionally substituted NH₂;
X₁ is selected from optionally substituted —CH₂—;
X₃ is selected from optionally substituted —CH═ or —N═;
R₂ is selected from hydrogen and optionally substituted C₁-C₆ alkyl; when R₂ comprises methylene units, the methylene units of R₂ are each independently unreplaced, or are each independently replaced by any structure;
W is absent, or W is selected from optionally substituted C₁-C₆ alkyl; when W comprises methylene units, the methylene units of W are each independently unreplaced, or are each independently replaced by any structure;
B is selected from the group consisting of: optionally substituted aryl and optionally substituted heteroaryl;
Z is selected from —N(R_{Z-1})—, wherein R_{Z-1} is selected from H or optionally substituted C₁-C₆ alkyl;
ring A is selected from: optionally substituted alcyl, optionally substituted aliphatic heterocyclyl, optionally substituted aryl, and optionally substituted heteroaryl;
R_{A-1} is selected from hydrogen, halogen, hydroxy, amino, and optionally substituted C₁-C₆ alkyl;
when R_{A-1} comprises methylene units, the methylene units of R_{A-1} are each independently unreplaced, or are each independently replaced by any structure;
m is selected from 1, 2, or 3;
L_{w} is -L₃-L₂-L₁;
L₁ is selected from 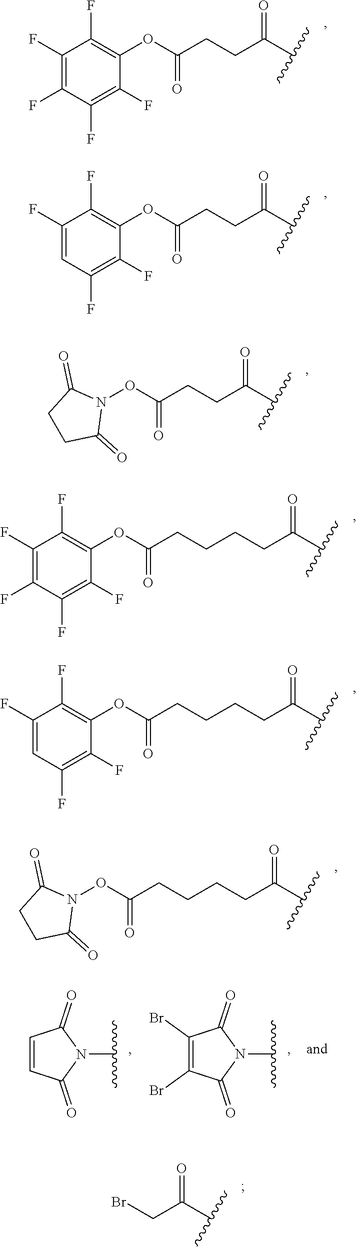
L₂ is selected from 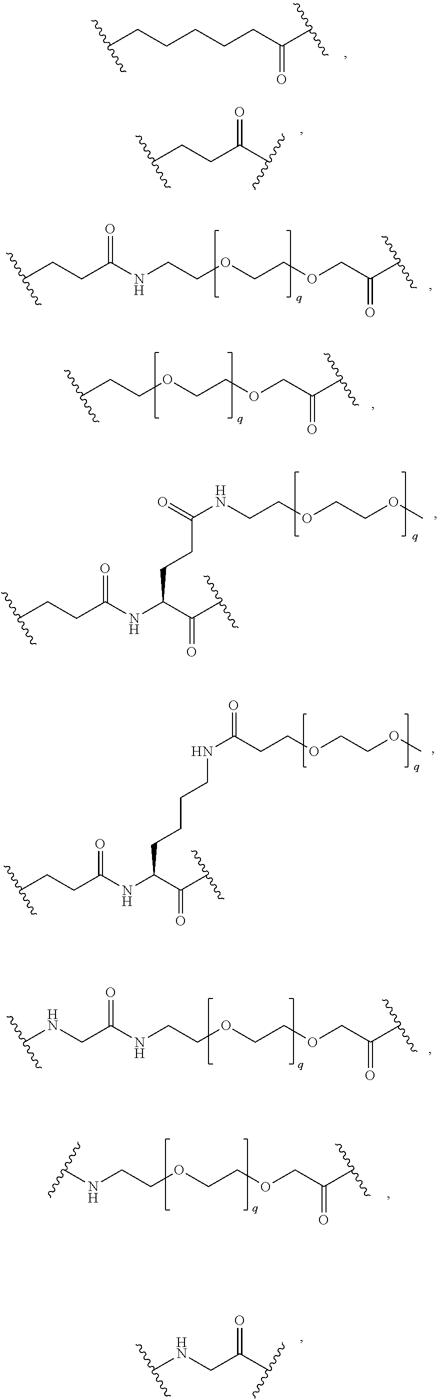
and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

38. The compound or the conjugate thereof, or the isomer thereof, or the mixture thereof, or the pharmaceutically acceptable salt thereof according to claim 37, wherein the compound is represented by formulas (II-1B-1) and (II-1B-2): 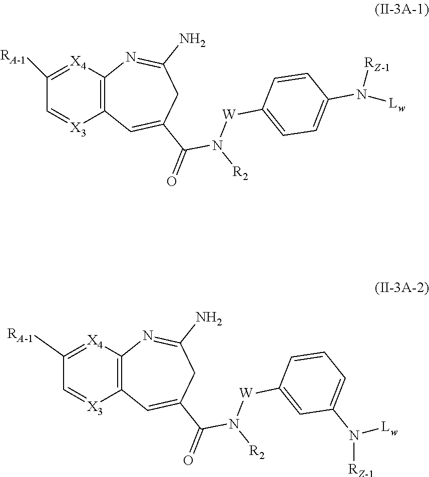
wherein
R_{Z-1} is selected from H or C₁-C₆ alkyl optionally substituted with 1, 2, or 3 fluorine, chlorine, bromine, iodine, hydroxy, or amino substituents;
X₄ and Xₛ are each independently selected from —CH═ and —N═;
R₂ is selected from C₁-C₆ alkyl optionally substituted with 1, 2, or 3 R₂₋₁; each R₂₋₁ is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R₂-2; each R₂₋₂ is independently selected from: hydrogen, halogen, hydroxy, or amino; the methylene units of R₂ are each independently unreplaced, replaced by —O—, or replaced by —N(R₂₋₃)—; R₂₋₃ is selected from: hydrogen or C₁-C₃ alkyl;
W is selected from C₁-C₆ alkyl optionally substituted with 1, 2, or 3 R_{w-1}; each R_{w-1} is independently selected from: hydrogen, halogen, hydroxy, amino, C₁-C₆ alkyl, and C₃-C₆ cycloalkyl, wherein the C₁-C₆ alkyl and the C₃-C₆ cycloalkyl are each independently optionally substituted with 1, 2, or 3 R_{w-2}; each R_{w-2} is independently selected from: hydrogen, halogen, hydroxy, or amino; the methylene units of W are each independently unreplaced, replaced by —O—, or replaced by —N(R_{w-3})—; R_{w-3} is selected from: hydrogen or C₁-C₃ alkyl;
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2};
each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆ alkyl), or —N(C₁-C₆ alkyl)₂;
L_{w} is selected from

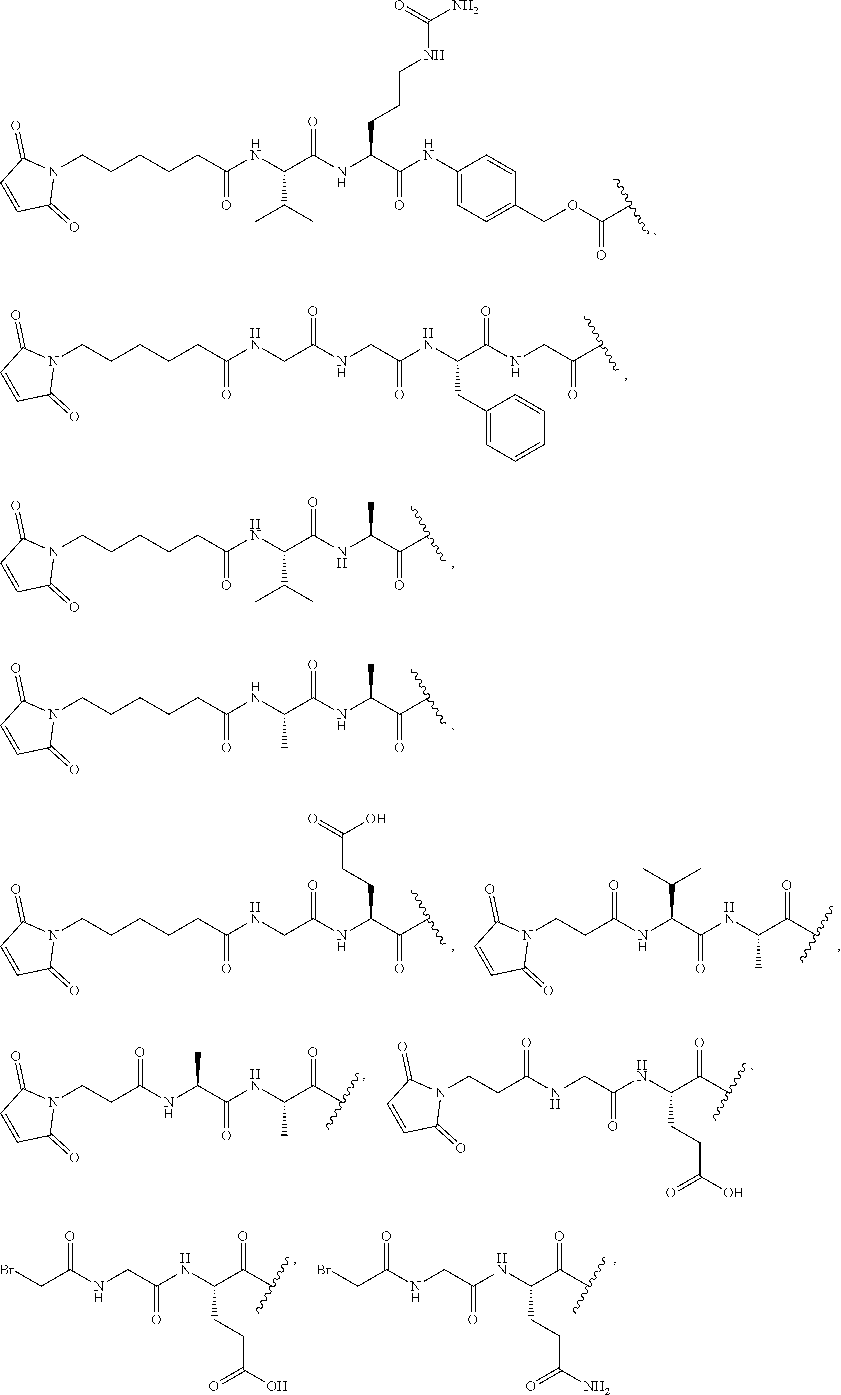

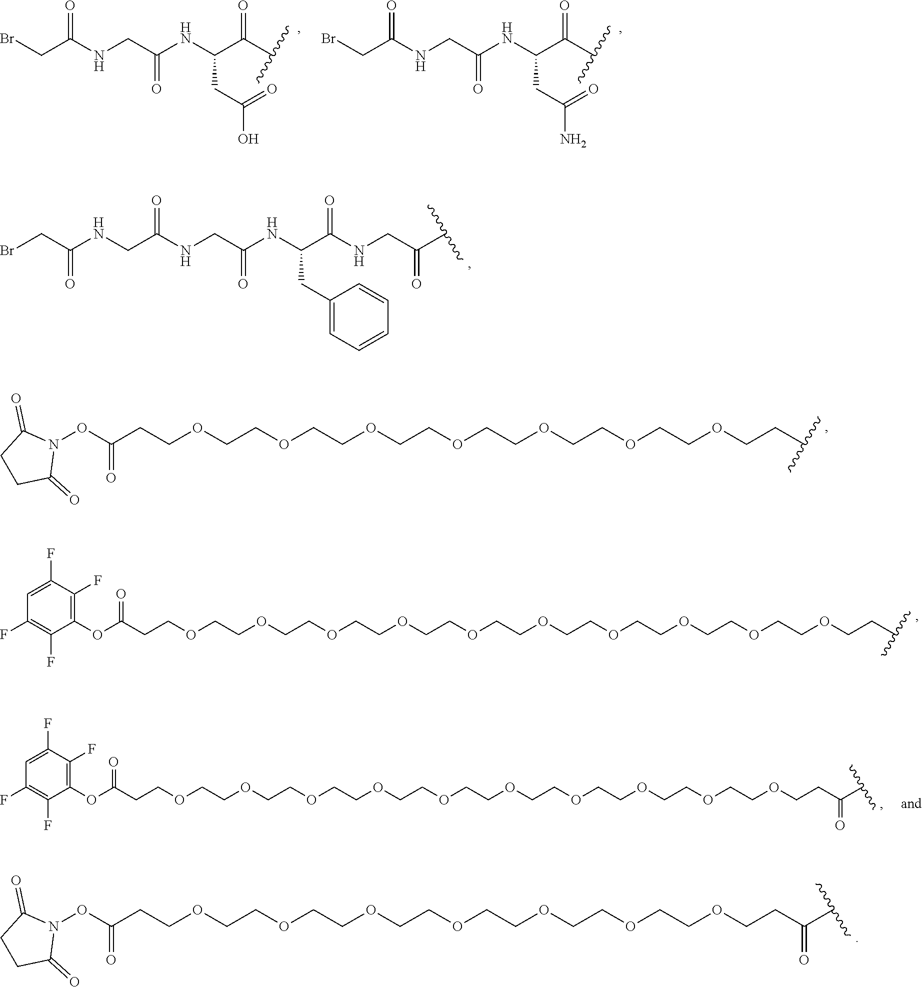

39. A compound or a conjugate thereof, or an isomer thereof, or a mixture thereof, or a pharmaceutically acceptable salt thereof, selected from:

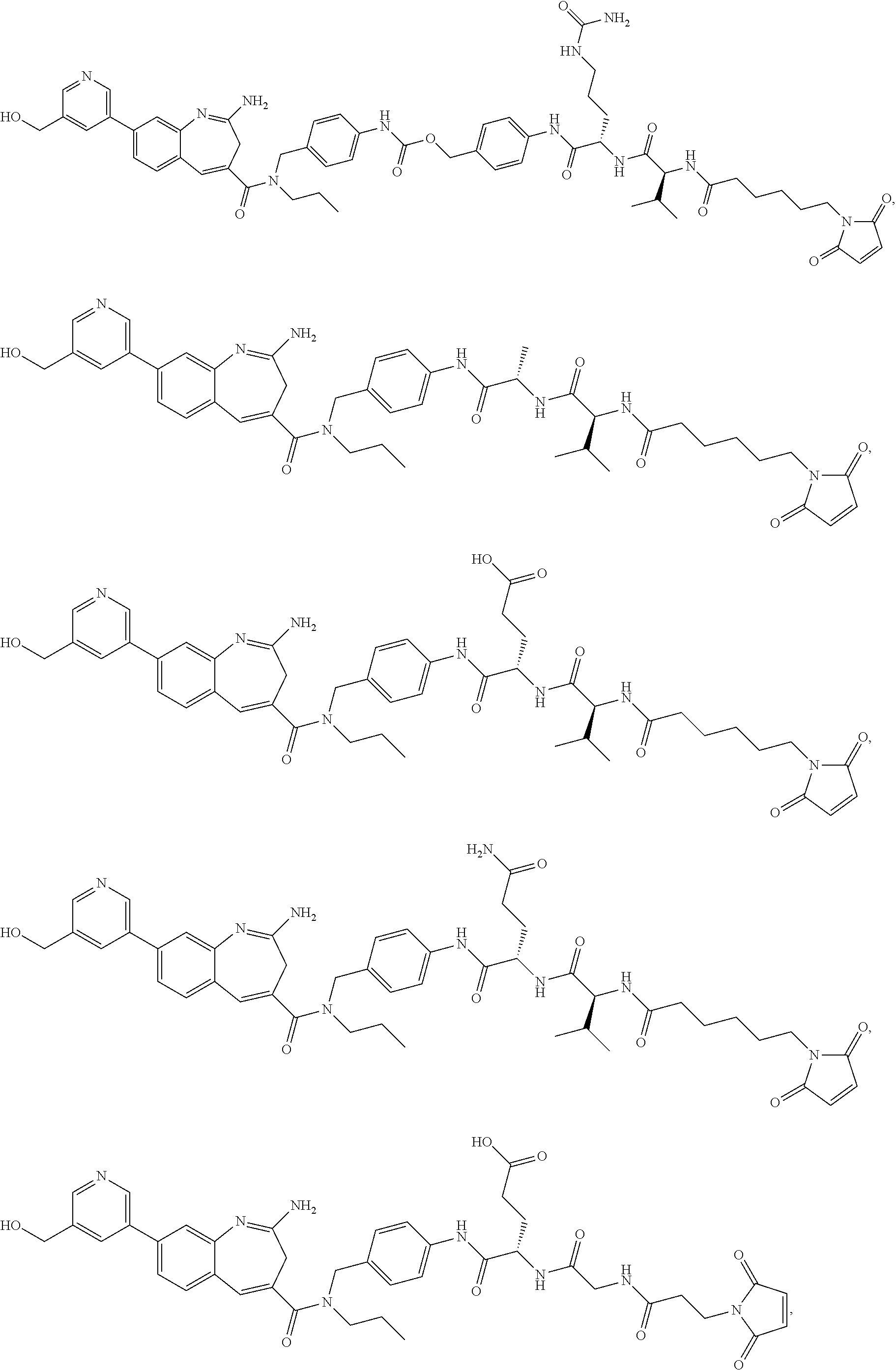

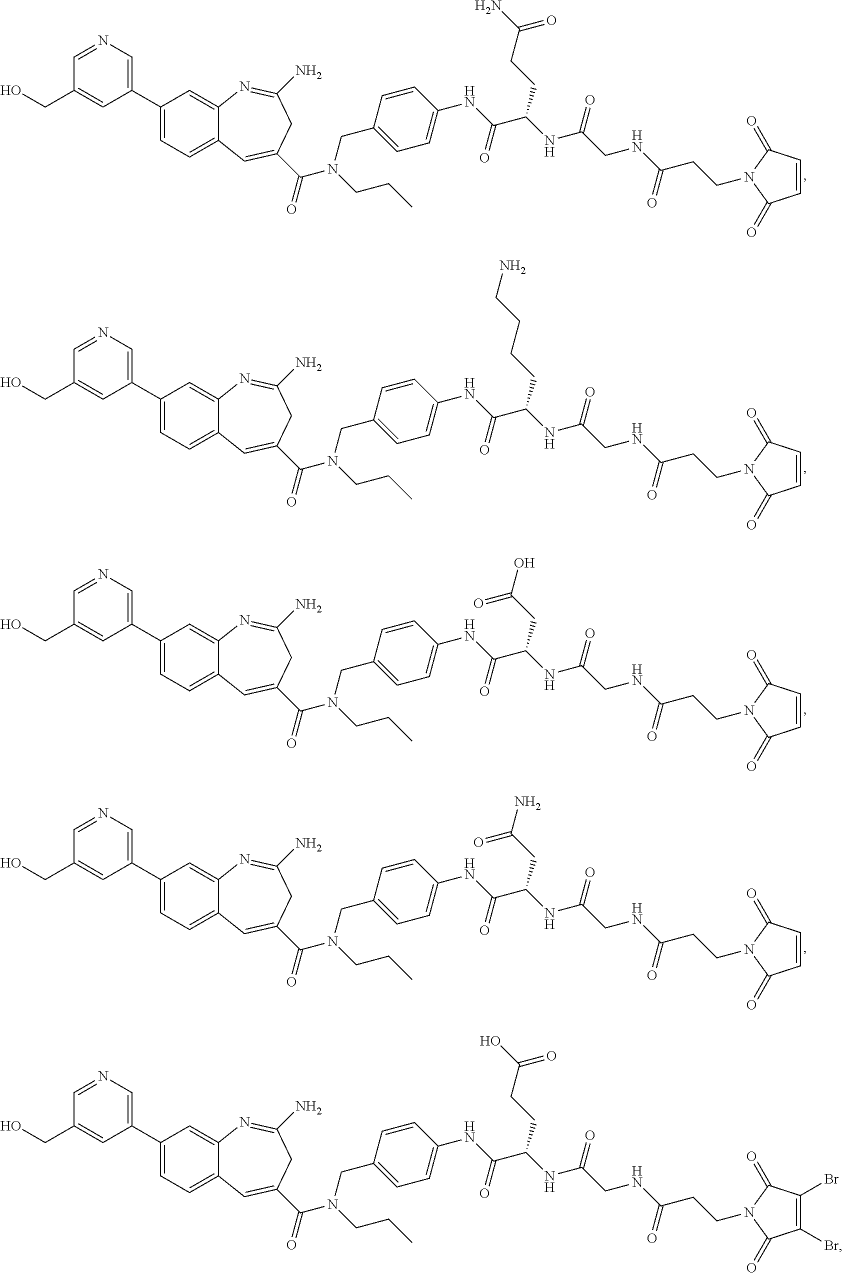

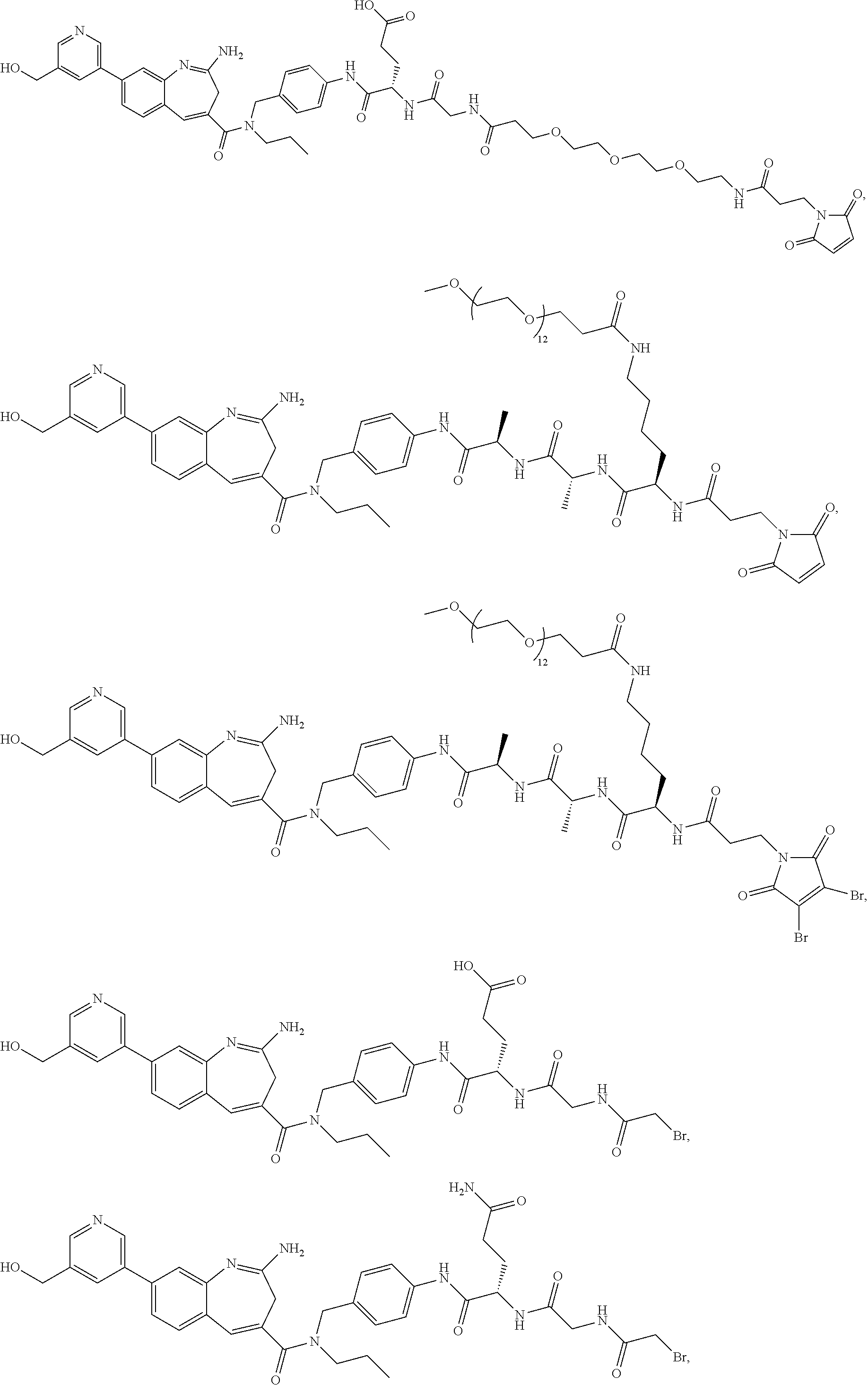

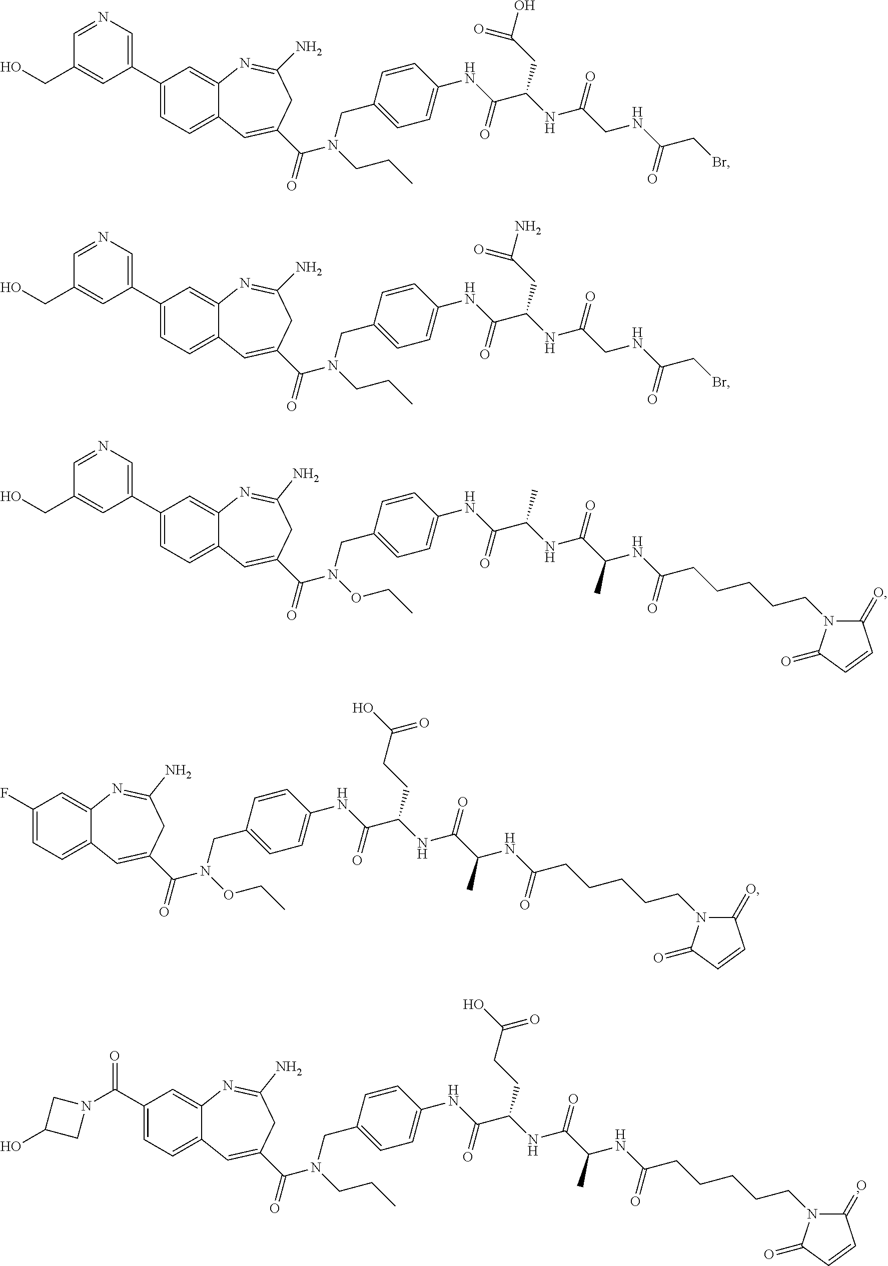

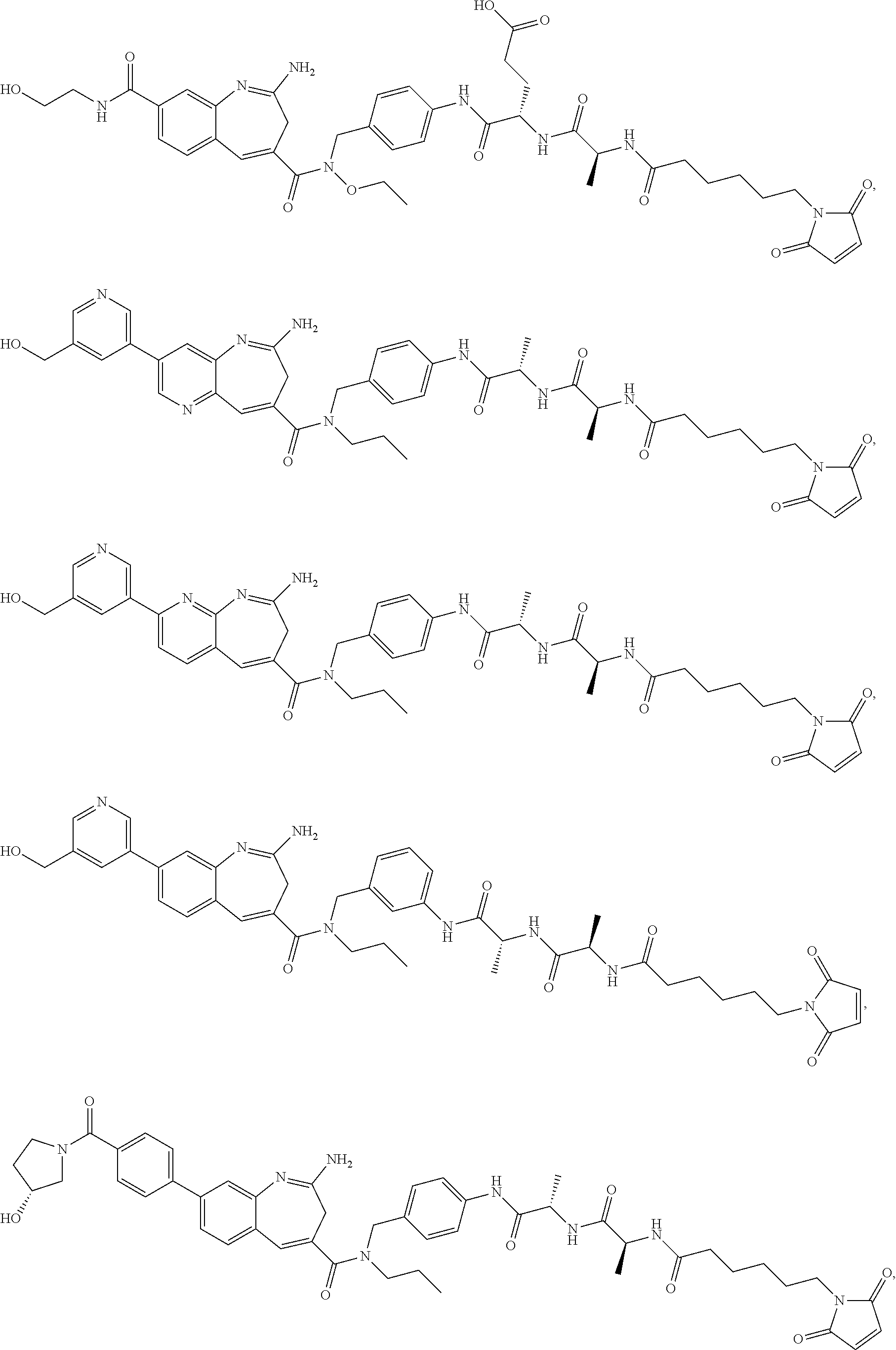

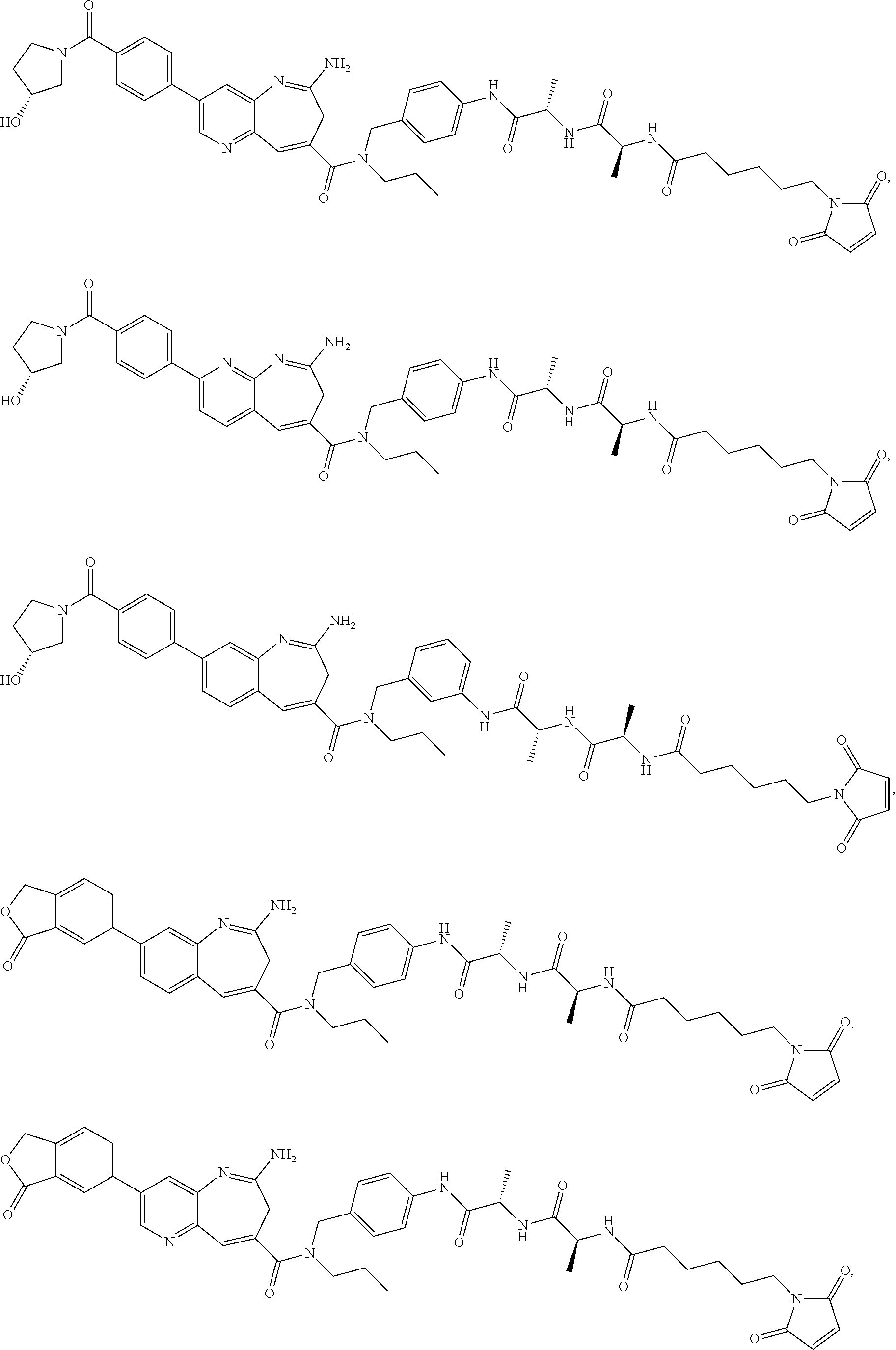

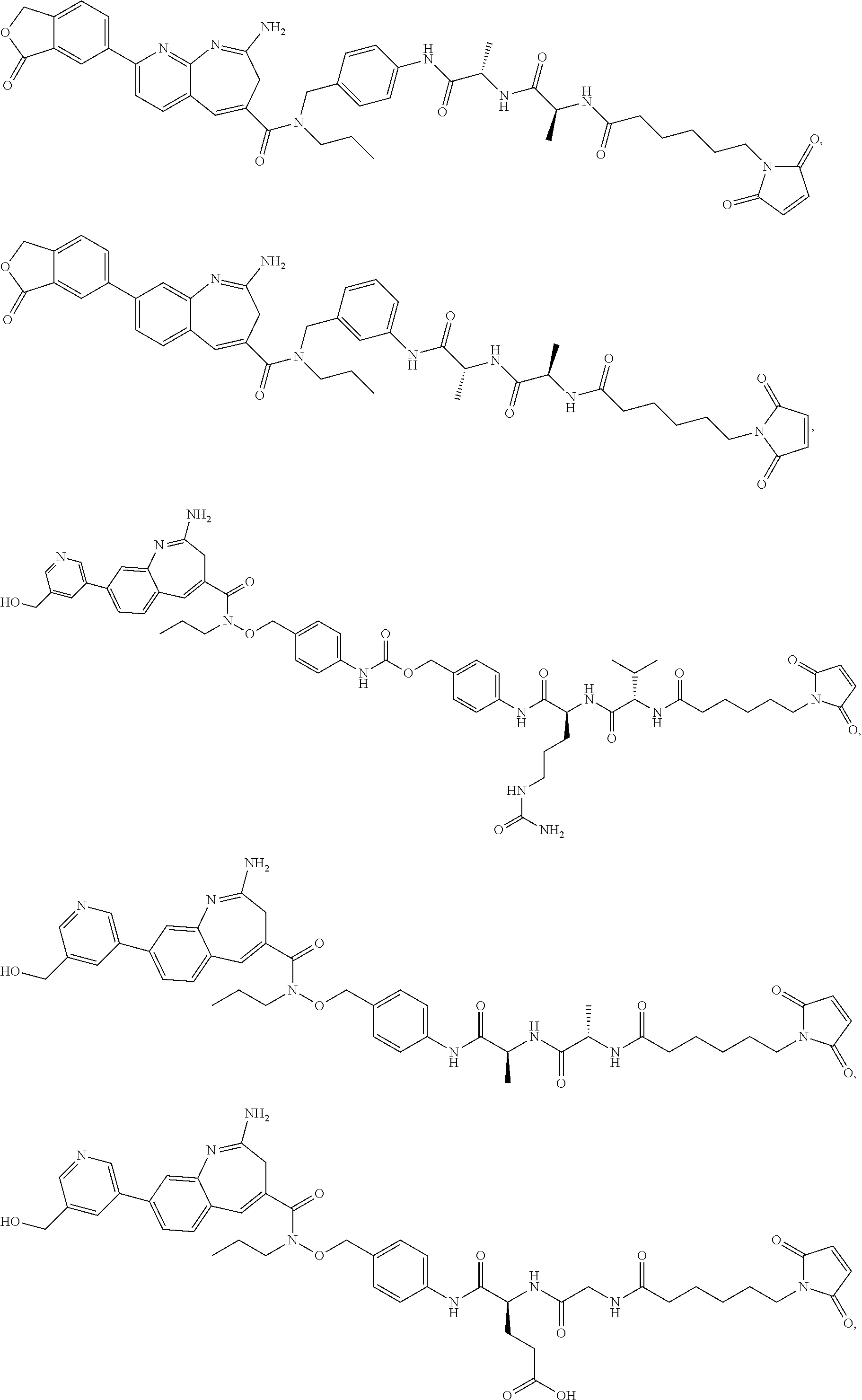

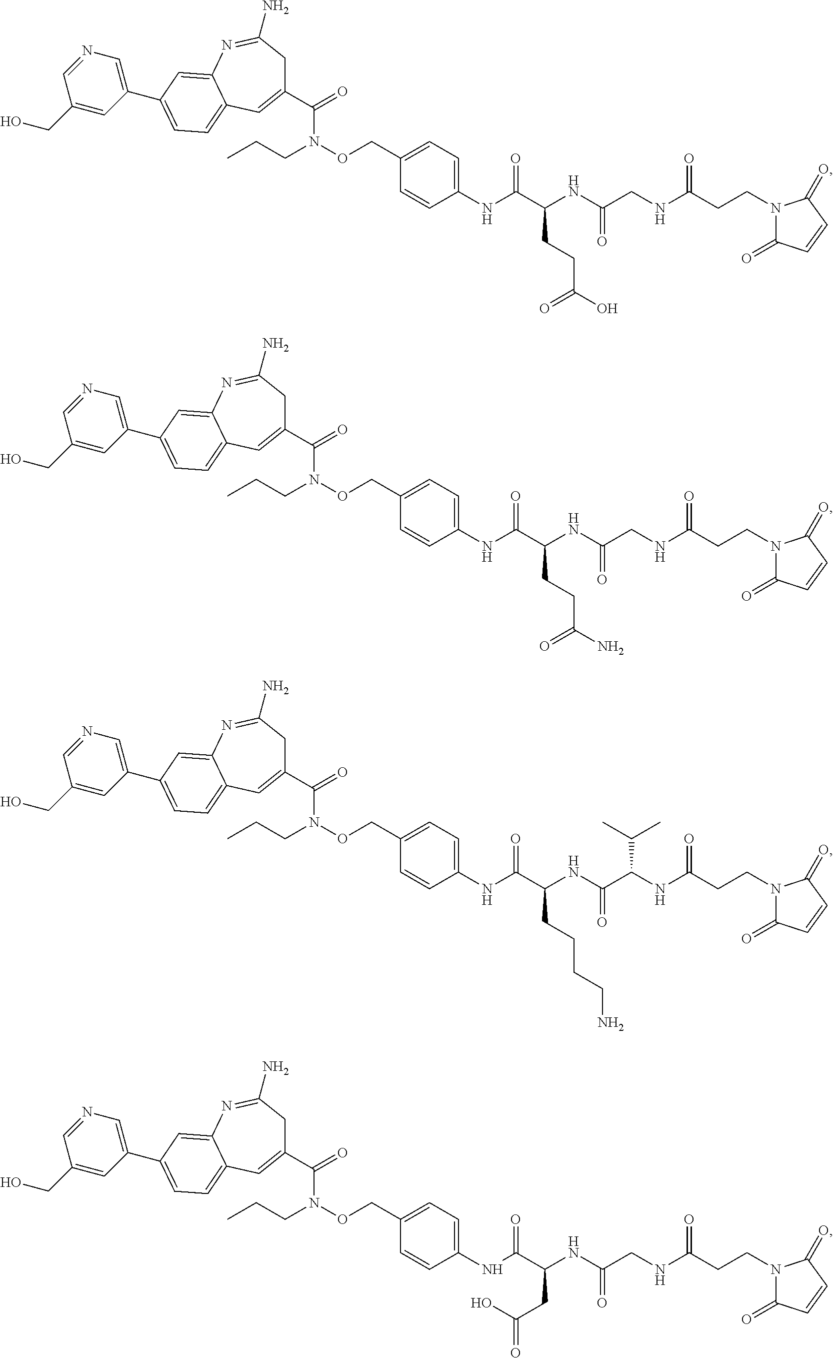 [SMILES: CCCN(OCc1ccc(NC(=O)[C@H](CCC(=O)O)NC(=O)CNC(=O)CCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(N)=O)NC(=O)CNC(=O)CCN2C(=O)C=CC2=O)cc1)C(=O)C1=Cc2ccc(-c3cncc(CO)c3)cc2N=C(N)C1 ; CCCN(OCc1ccc(NC(=O)[C@H](CCCCN)NC(=O)[C@@H](NC(=O)CCN2C(=O)C=CC2=O)C(C)C)cc1)C(=O)C1=Cc2ccc(-c3cncc(CO)c3)cc2N=C(N)C1 ; CCCN(OCc1ccc(NC(=O)[C@H](CC(=O)O)NC(=O)CNC(=O)CCN2C(=O)C=CC2=O)cc1)C(=O)C1=Cc2ccc(-c3cncc(CO)c3)cc2N=C(N)C1]

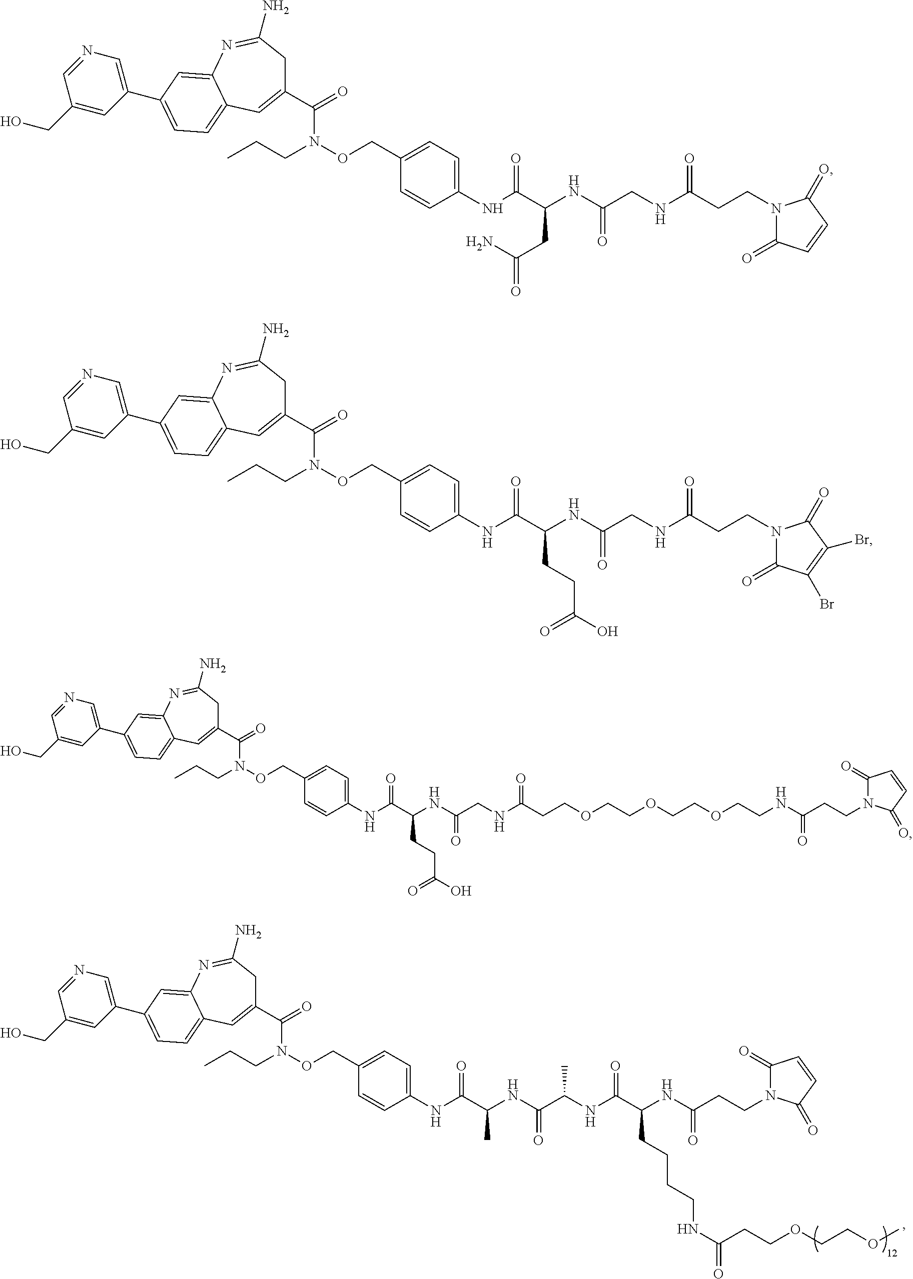

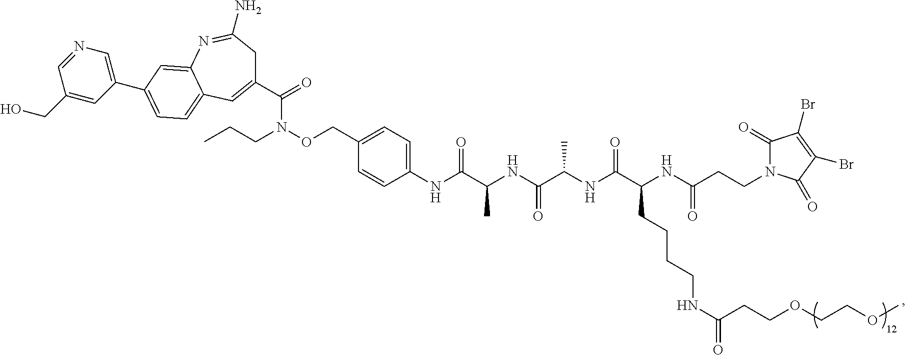

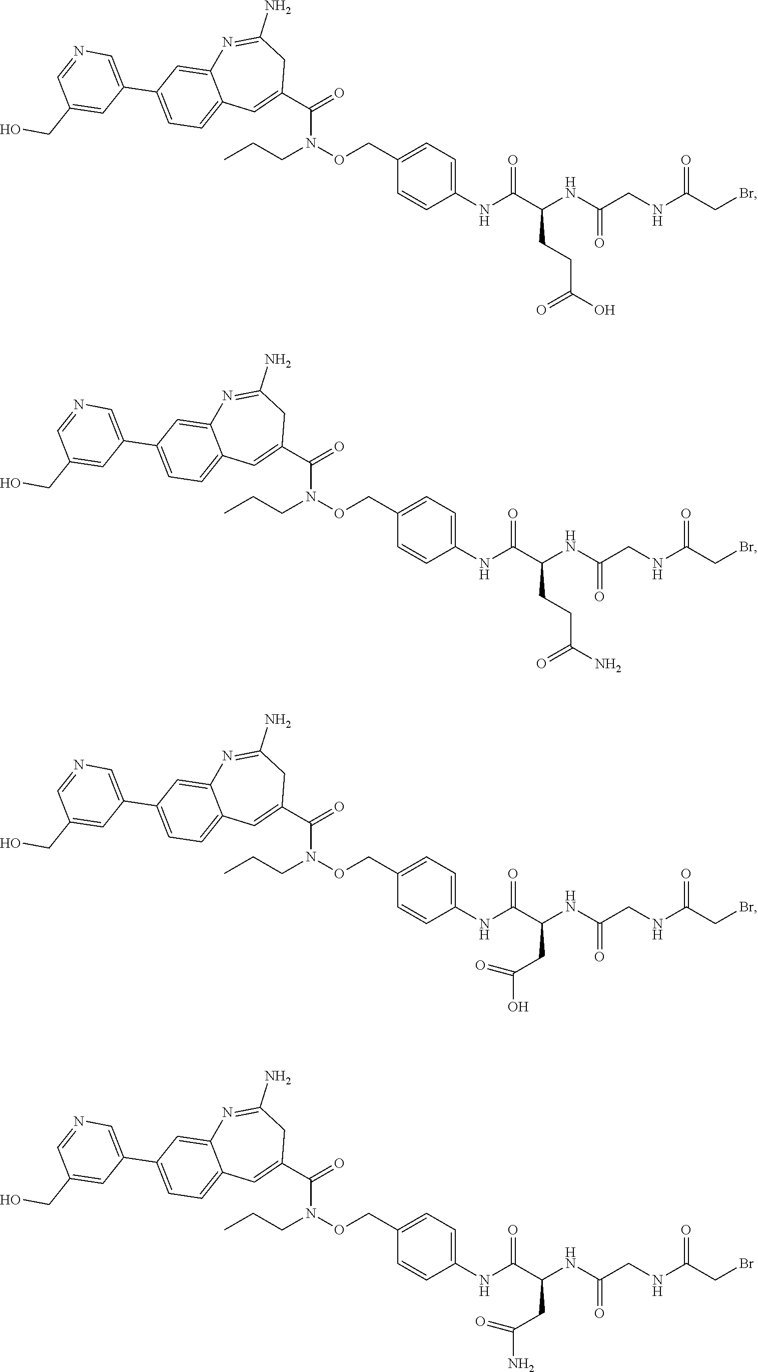 [SMILES: CCCN(OCc1ccc(NC(=O)[C@H](CCC(=O)O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CCC(N)=O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CC(=O)O)NC(=O)CNC(=O)CBr)cc1)C(=O)C1=Cc2ccc(-c3cncc(CO)c3)cc2N=C(N)C1 ; CCCN(OCc1ccc(NC(=O)[C@H](CC(N)=O)NC(=O)CNC(=O)CBr)cc1)C(=O)C1=Cc2ccc(-c3cncc(CO)c3)cc2N=C(N)C1]

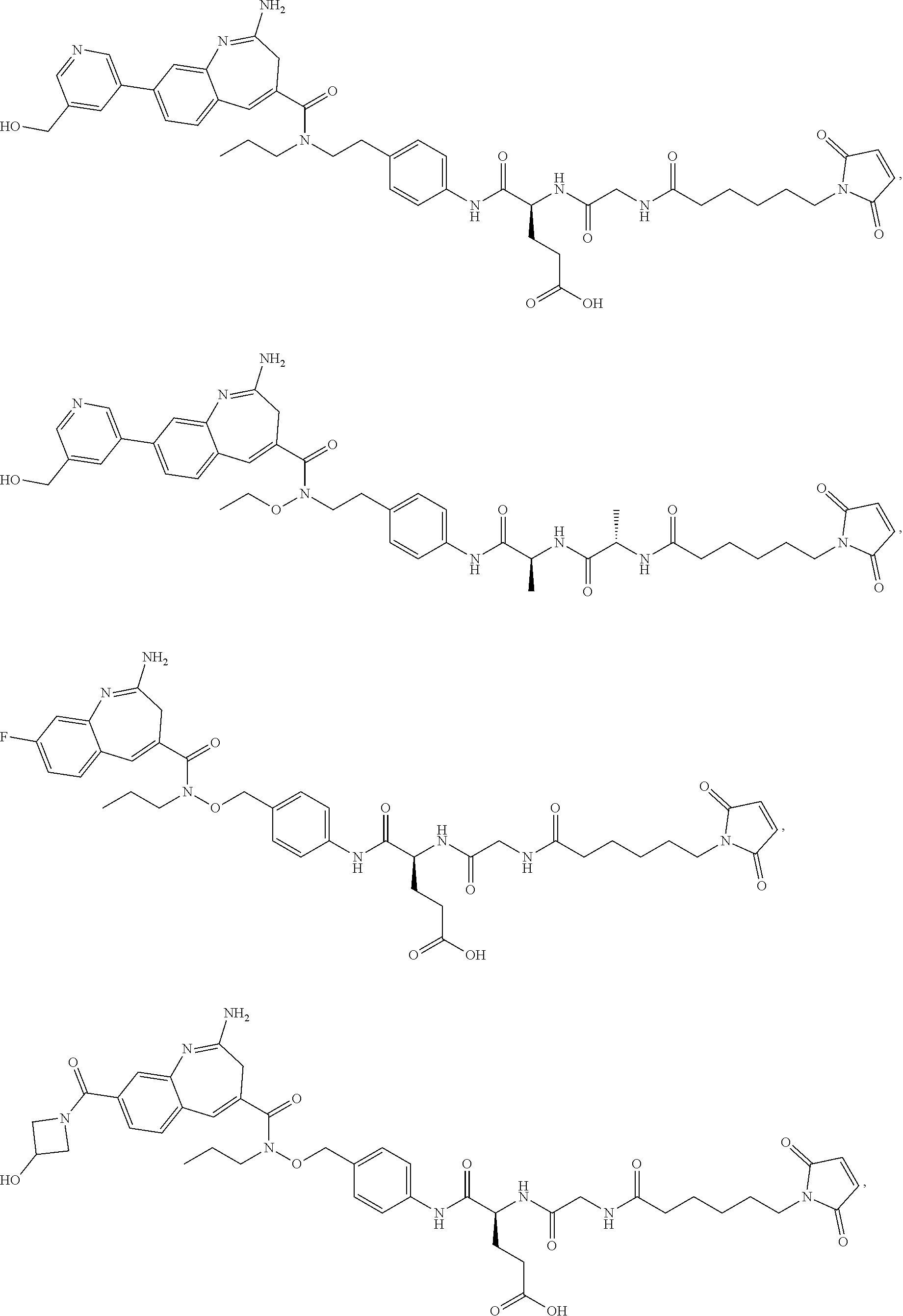 [SMILES: CCCN(CCc1ccc(NC(=O)[C@H](CCC(=O)O)NC(=O)CNC(=O)CCCCCN2C(=O)C=CC2=O)cc1)C(=O)C1=Cc2ccc(-c3cncc(CO)c3)cc2N=C(N)C1 ; CCON(CCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(OCc1ccc(NC(=O)[C@H](CCC(=O)O)NC(=O)CNC(=O)CCCCCN2C(=O)C=CC2=O)cc1)C(=O)C1=Cc2ccc(F)cc2N=C(N)C1 ; CCCN(OCc1ccc(NC(=O)[C@H](CCC(=O)O)NC(=O)CNC(=O)CCCCCN2C(=O)C=CC2=O)cc1)C(=O)C1=Cc2ccc(C(=O)N3CC(O)C3)cc2N=C(N)C1]

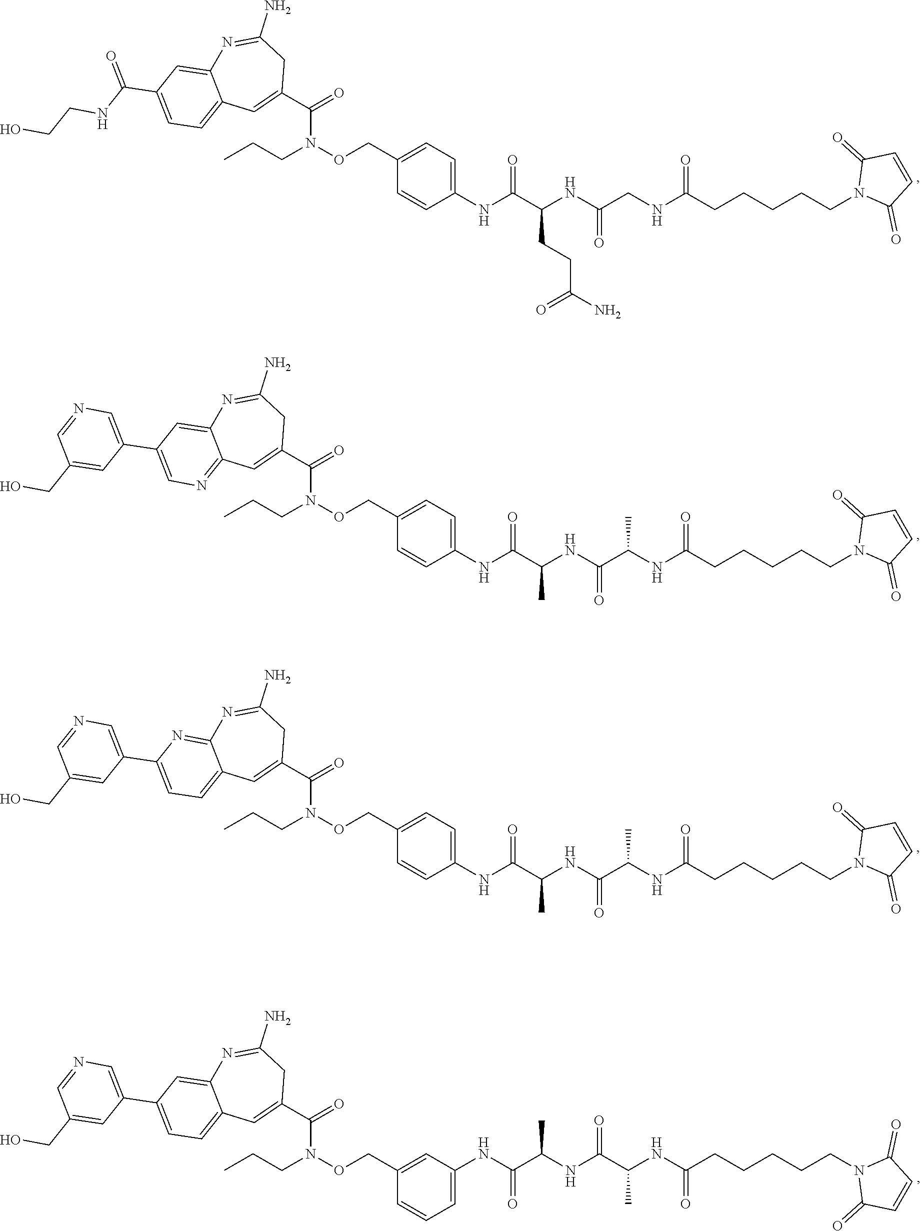 [SMILES: CCCN(OCc1ccc(NC(=O)[C@H](CCC(N)=O)NC(=O)CNC(=O)CCCCCN2C(=O)C=CC2=O)cc1)C(=O)C1=Cc2ccc(C(=O)NCCO)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)nc2N=C(N)C1 ; CCCN(OCc1cccc(NC(=O)[C@@H](C)NC(=O)[C@@H](C)NC(=O)CCCCCN2C(=O)C=CC2=O)c1)C(=O)C1=Cc2ccc(-c3cncc(CO)c3)cc2N=C(N)C1]

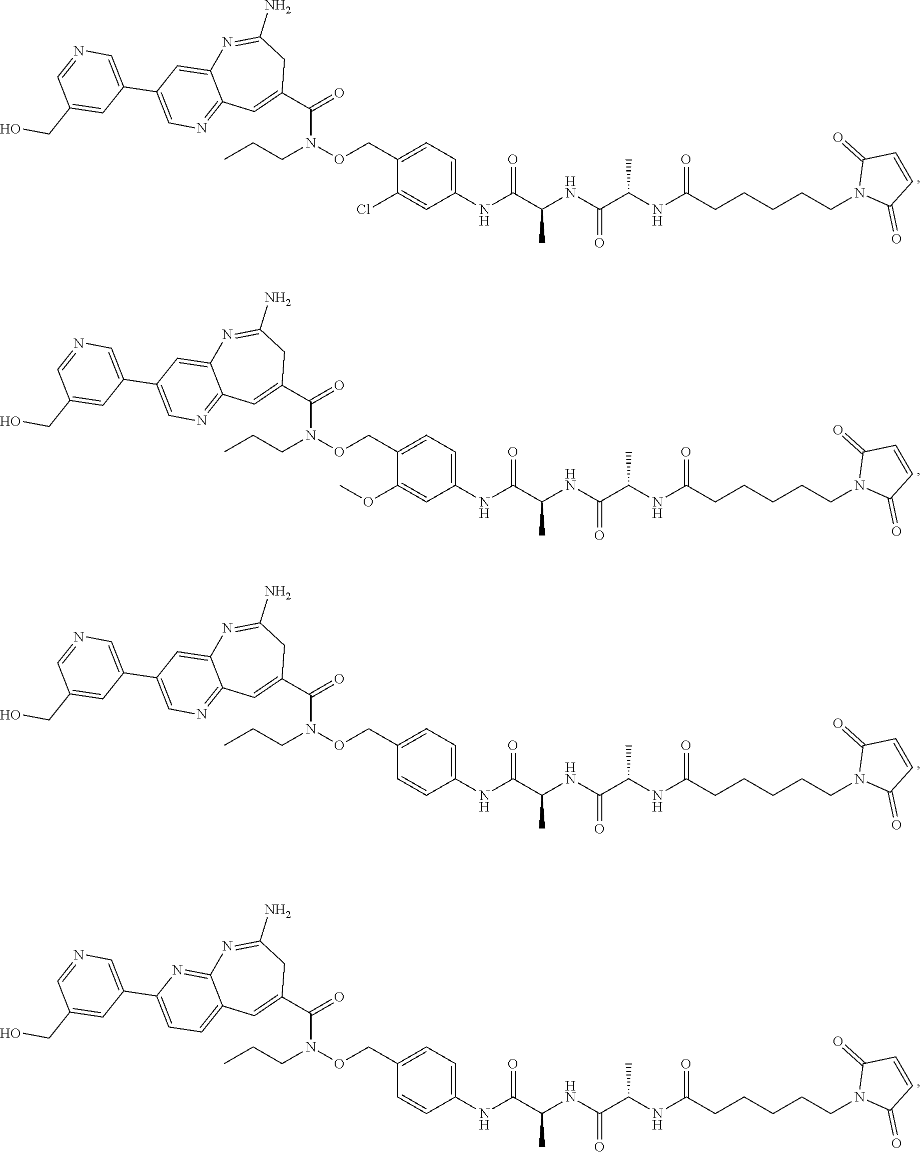 [SMILES: CCCN(OCc1ccc(NC(=O)[C@H](C)NC(=O)[C@H](C)NC(=O)CCCCCN2C(=O)C=CC2=O)cc1Cl)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1OC)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ncc(-c3cncc(CO)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)nc2N=C(N)C1]

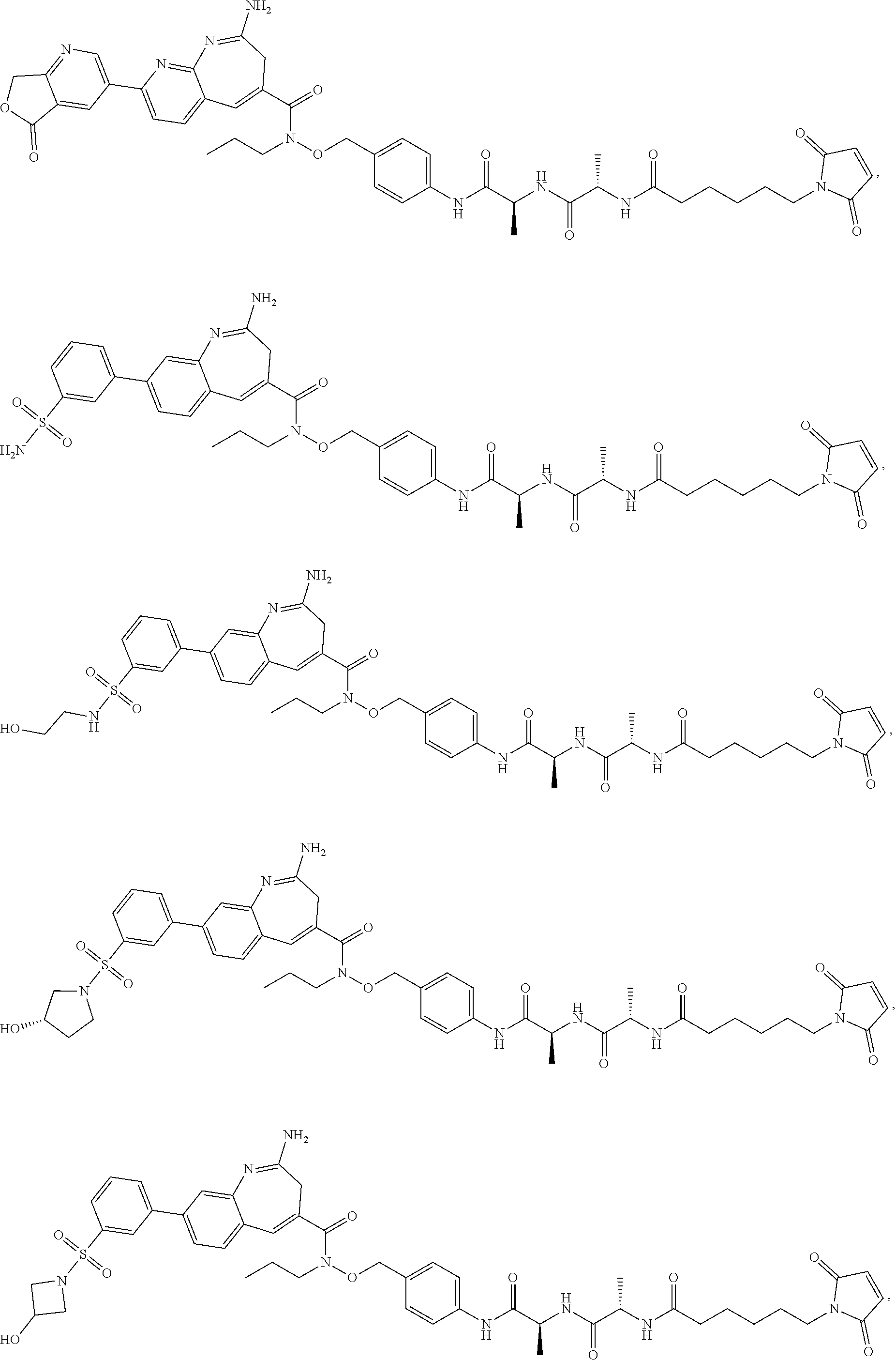

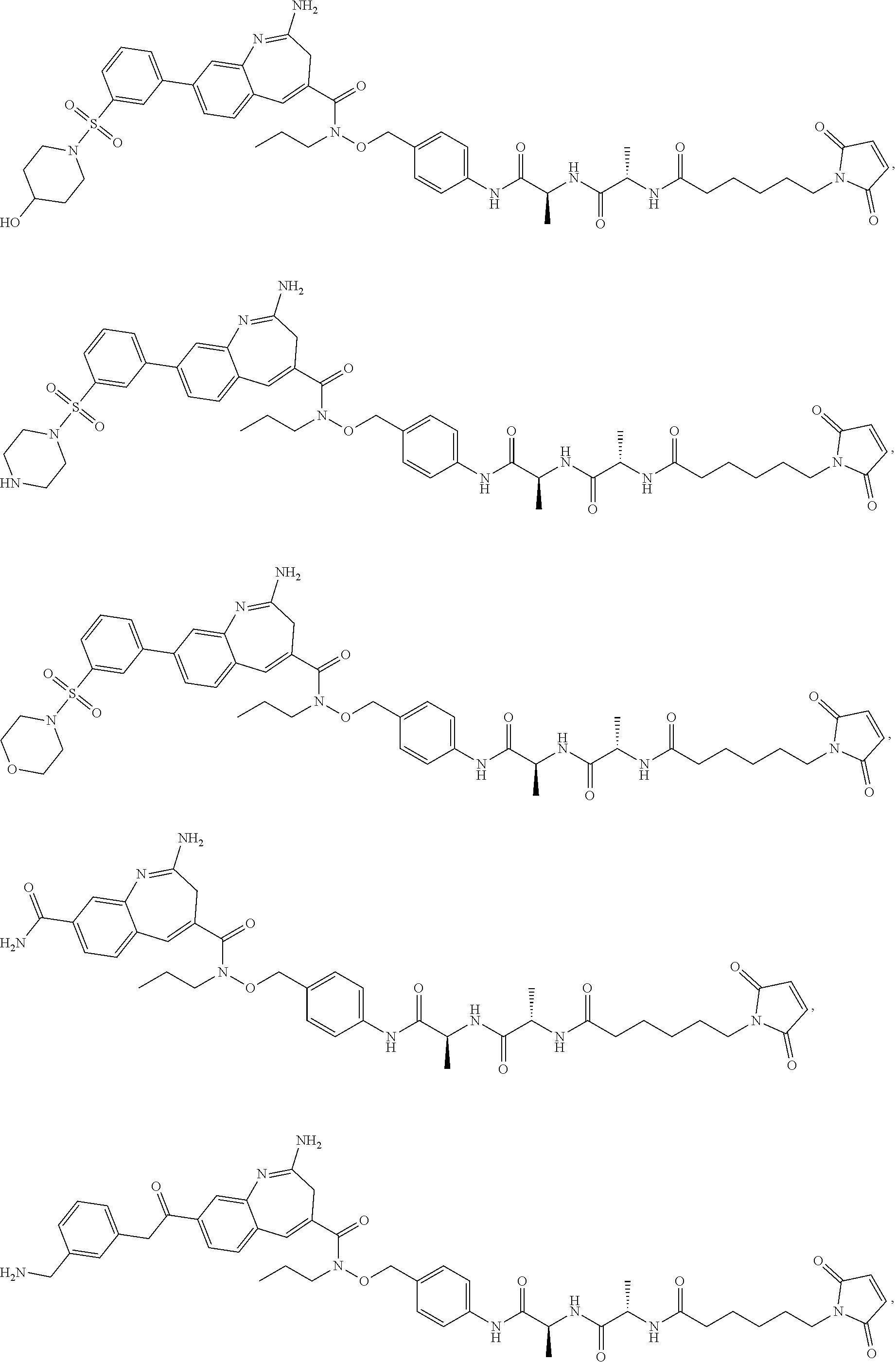

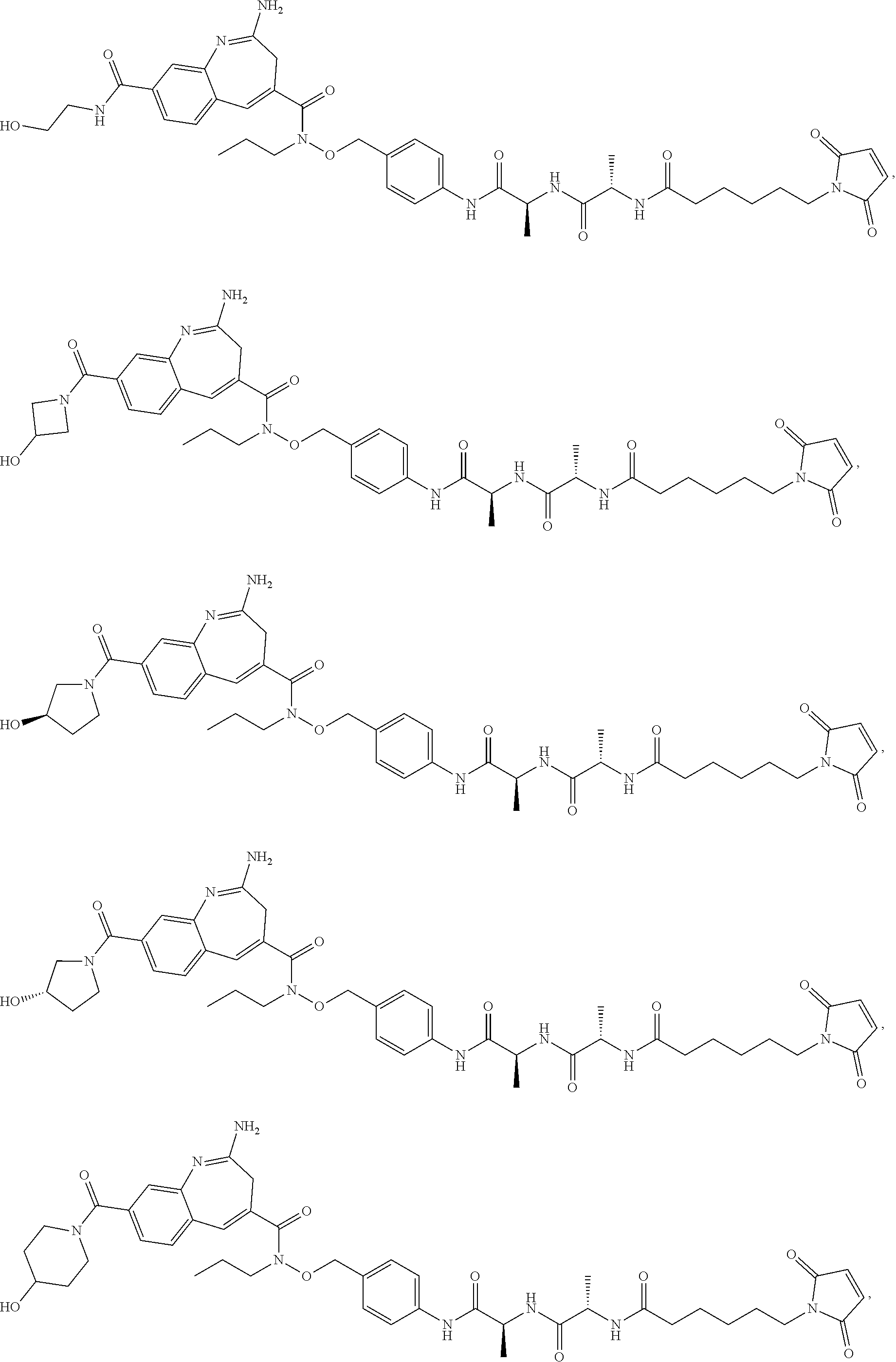

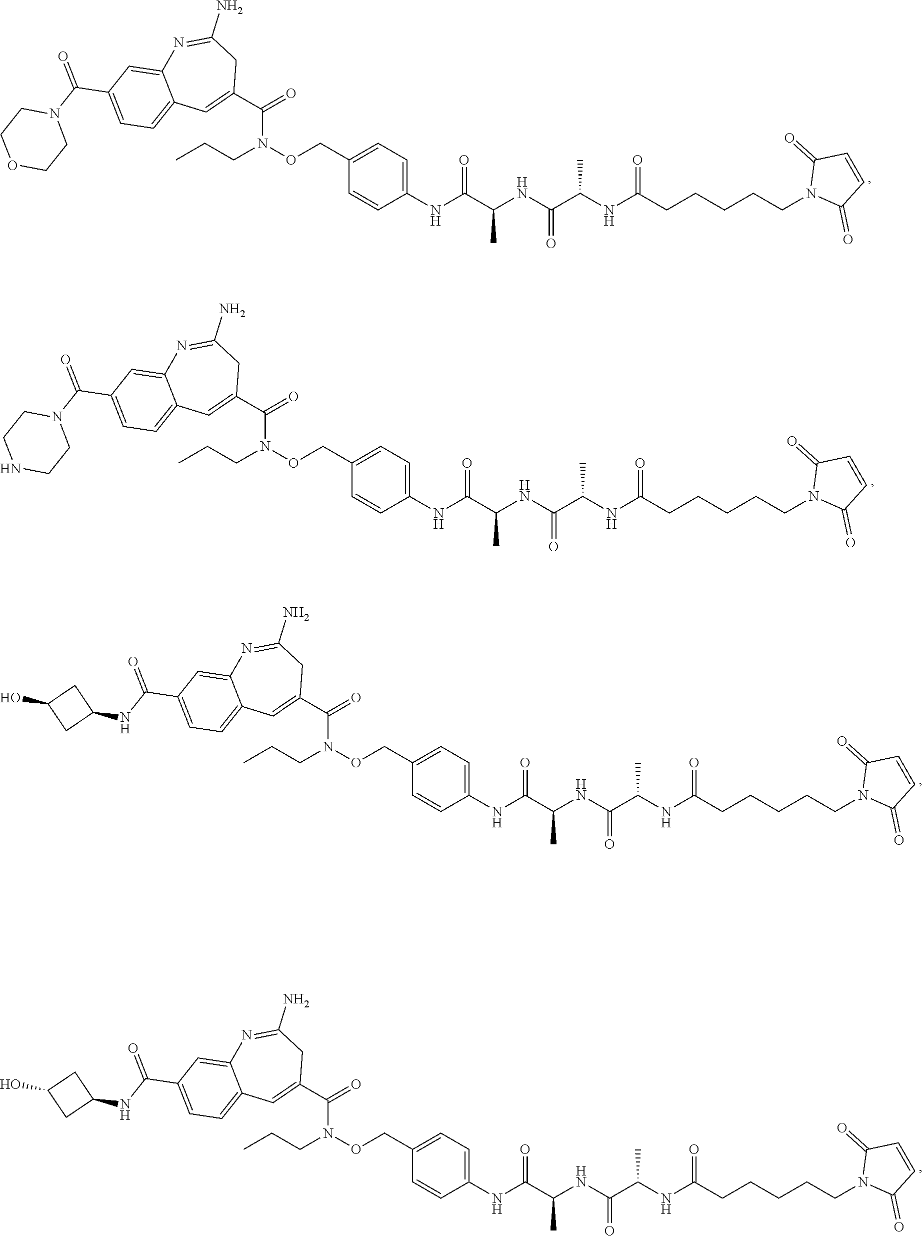 [SMILES: CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N3CCOCC3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N3CCNCC3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N[C@H]3C[C@@H](O)C3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N[C@H]3C[C@H](O)C3)cc2N=C(N)C1]

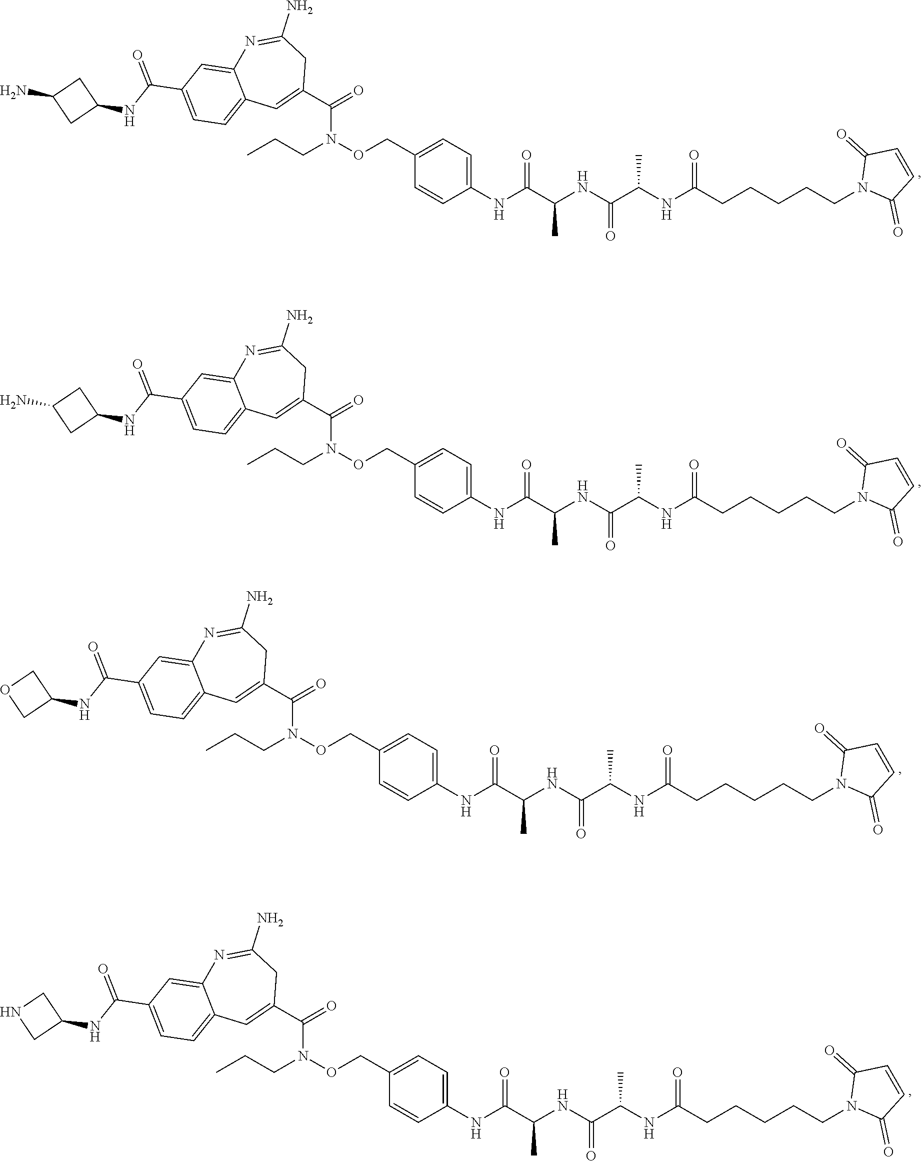

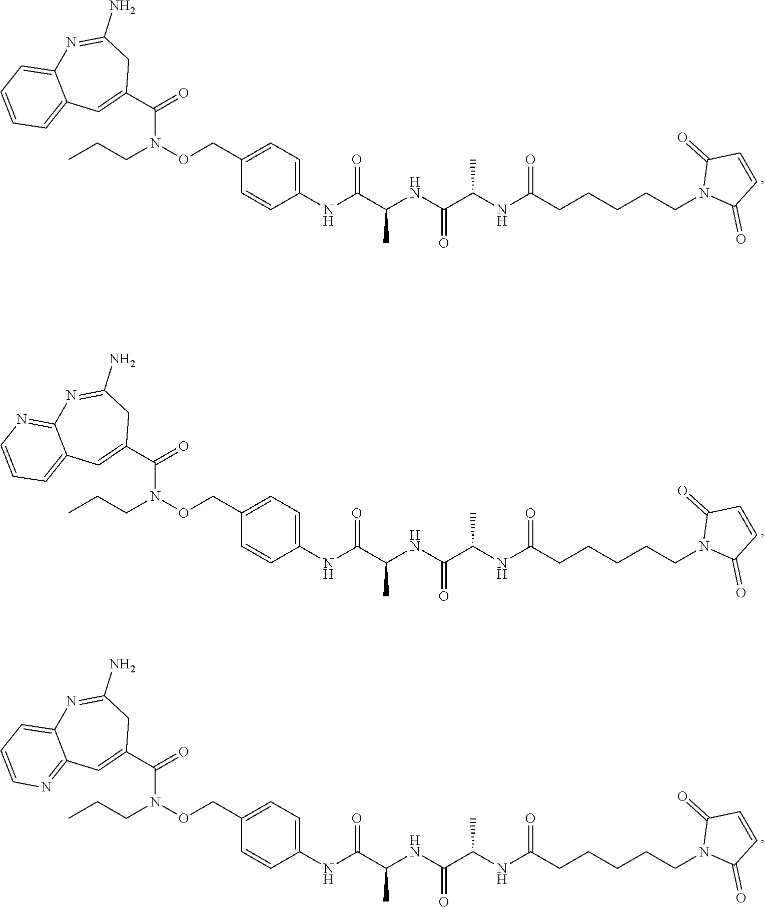 [SMILES: CCCN(OCc1ccc(NC(=O)[C@H](C)NC(=O)[C@H](C)NC(=O)CCCCCN2C(=O)C=CC2=O)cc1)C(=O)C1=Cc2ccccc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2cccnc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ncccc2N=C(N)C1]

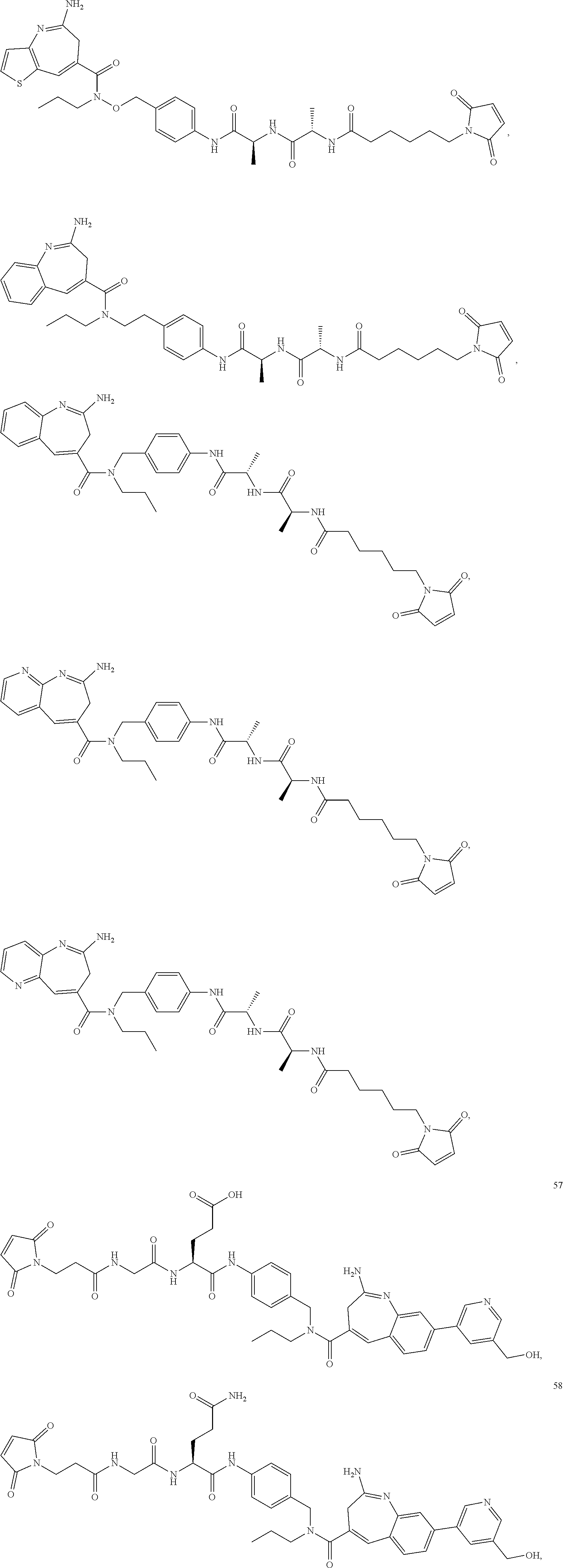

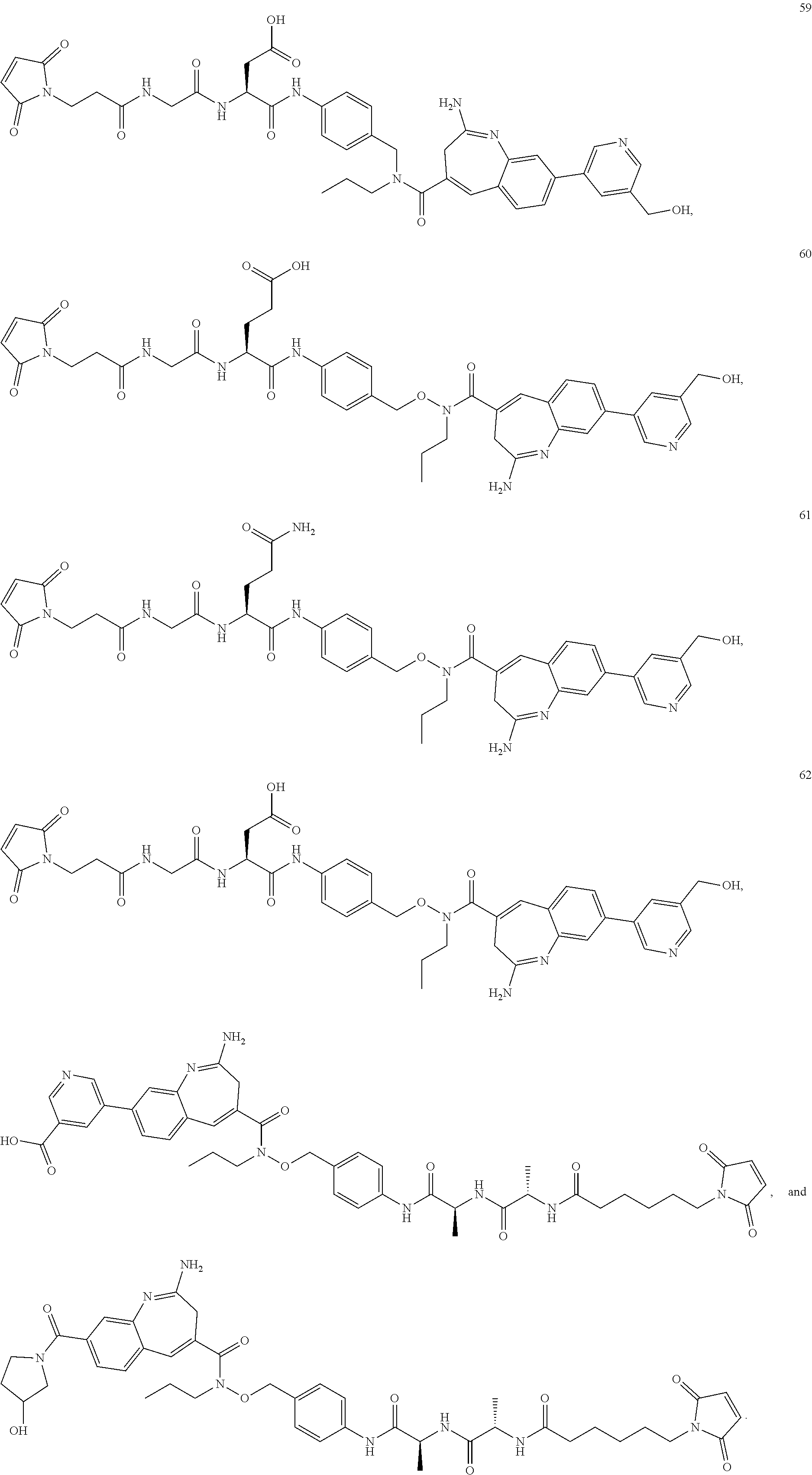

40. A ligand-drug conjugate, or an isomer thereof, or a mixture thereof, or a pharmaceutically acceptable salt thereof, wherein the ligand-drug conjugate comprises a structure represented by formula (III-2A): 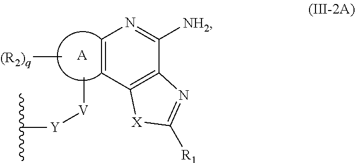
wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—, 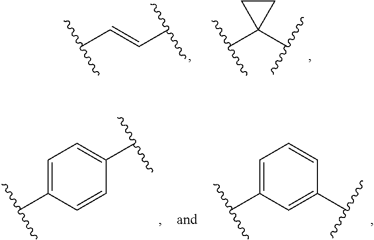
R_{V4} being selected from H, C₁-C₆ alkyl, and 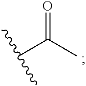 [SMILES: *C(C)=O]
Y is selected from —N(R_{y1})—, —O—, —S—, 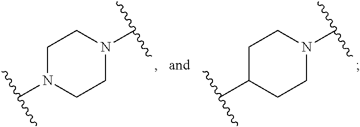 [SMILES: *N1CCN(*)CC1 ; *C1CCN(*)CC1]
R_{Y1} is selected from hydrogen and C₁-C₆ alkyl;
q is selected from 0, 1, 2, or 3;
p is selected from 1, 2, 3, 4, 5, 7, and 8;
the wavy line [symbol] in the general formula represents being directly linked to a ligand via a nitrogen atom or an oxygen atom on the Y group, or being linked to the ligand via a linker unit.

41. The ligand-drug conjugate, or the isomer thereof, or the mixture thereof, or the pharmaceutically acceptable salt thereof according to claim 40, wherein the ligand-drug conjugate comprises a structure represented by formula (III-2B): 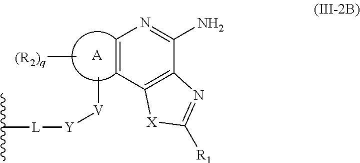
wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—, 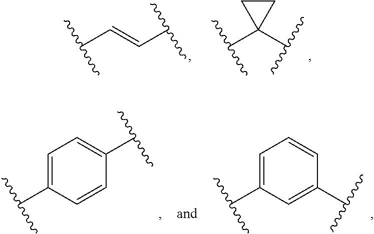
R_{V4} being selected from H, C₁-C₆ alkyl, and 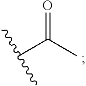 [SMILES: *C(C)=O]
Y is selected from —N(R_{Y1})—, —O—, —S—, 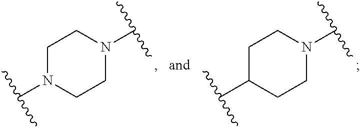 [SMILES: *N1CCN(*)CC1 ; *C1CCN(*)CC1]
R_{Y1} is selected from hydrogen and C₁-C₆ alkyl;
q is selected from 0, 1, 2, or 3;
p is selected from 1, 2, 3, 4, 5, 7, and 8;
L is a linker unit;
the wavy line [symbol] in the general formula represents being linked to the ligand via the nitrogen atom or the carbon atom on the L group.

42. The ligand-drug conjugate, or the isomer thereof, or the mixture thereof, or the pharmaceutically acceptable salt thereof according to claim 40, wherein the ligand-drug conjugate comprises a structure represented by formula (III-2C): 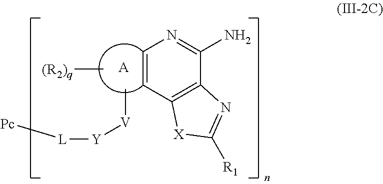
wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, Ri-2 being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—, 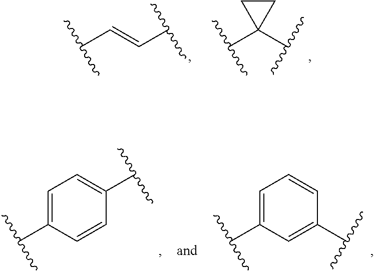
R_{V4} being selected from H, C₁-C₆ alkyl, and 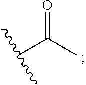 [SMILES: *C(C)=O]
Y is selected from —N(R_{Y1})—, —O—, —S—, 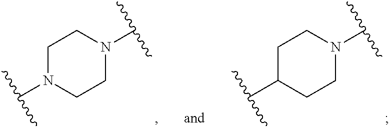 [SMILES: *N1CCN(*)CC1 ; *C1CCN(*)CC1]
R_{Y1} is selected from hydrogen and C₁-C₆ alkyl;
q is selected from 0, 1, 2, or 3;
p is selected from 1, 2, 3, 4, 5, 7, and 8;
L is a linker unit;
n is an integer or a decimal from 1 to 10;
Pc is a ligand.

43. The ligand-drug conjugate, or the isomer thereof, or the mixture thereof, or the pharmaceutically acceptable salt thereof according to any one of claims 40-42, wherein R₁ is selected from C₁-C₆ alkyl optionally substituted with 1, 2, or 3 R₁₋₁, each R₁₋₁ being independently selected from: hydrogen, chlorine, bromine, iodine, hydroxy, amino, —CH₃, —CH₂CH₃, and 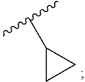 [SMILES: *C1CC1]
the methylene units of R₁ are each independently unreplaced or replaced by a group selected from: —O—, —N(CH₃)—, and —NH—.

44. The ligand-drug conjugate, or the isomer thereof, or the mixture thereof, or the pharmaceutically acceptable salt thereof according to any one of claims 40-43, wherein R₁ is selected from 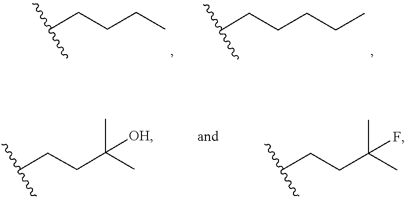
wherein the methylene units of R₁ are each independently unreplaced or replaced by a group selected from: —O—, —N(CH₃)—, and —NH—.

45. The ligand-drug conjugate, or the isomer thereof, or the mixture thereof, or the pharmaceutically acceptable salt thereof according to any one of claims 40-44, wherein R₁ is selected from

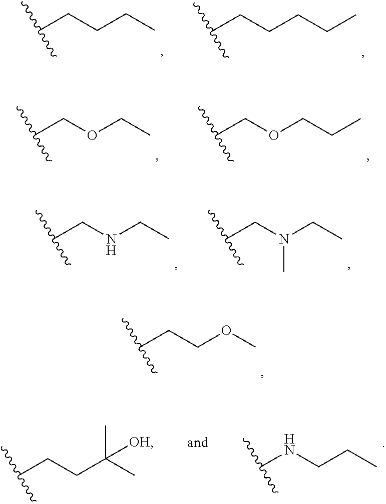

46. The ligand-drug conjugate, or the isomer thereof, or the mixture thereof, or the pharmaceutically acceptable salt thereof according to any one of claims 40-42, wherein R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂₋₁, each R₂₋₁ being independently selected from: hydrogen, halogen, hydroxy, and amino.

47. The ligand-drug conjugate, or the isomer thereof, or the mixture thereof, or the pharmaceutically acceptable salt thereof according to any one of claims 40-46, wherein R₂ is selected from hydrogen, chlorine, bromine, iodine, hydroxy, amino, sulfhydryl, —CH₃, —CH₂CH₃, and —OCH₃.

48. The ligand-drug conjugate, or the isomer thereof, or the mixture thereof, or the pharmaceutically acceptable salt thereof according to any one of claims 40-42, wherein ring A is selected from phenyl, pyridinyl, pyrrolyl, thienyl, furanyl, pyridazinyl, pyrimidinyl, and pyrazinyl.

49. The ligand-drug conjugate, or the isomer thereof, or the mixture thereof, or the pharmaceutically acceptable salt thereof according to any one of claims 40-48, wherein ring A is selected from phenyl or pyridinyl.

50. The ligand-drug conjugate, or the isomer thereof, or the mixture thereof, or the pharmaceutically acceptable salt thereof according to any one of claims 40-42, wherein V is selected from —(C(R_{V1})(R_{V2}))ₚ—, wherein R_{A1} and R_{A2} are each independently selected from hydrogen, chlorine, bromine, iodine, hydroxy, amino, —CH₃, —CH₂CH₃, and 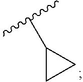 [SMILES: *C1CC1]
the methylene units of V are each independently unreplaced or replaced by a group selected from: —O—, —NH—, —CO—, —NHCO—,

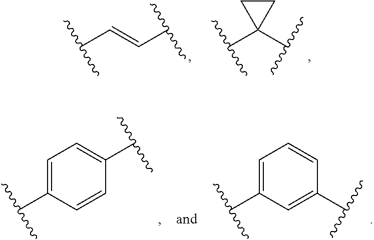

51. The ligand-drug conjugate, or the isomer thereof, or the mixture thereof, or the pharmaceutically acceptable salt thereof according to any one of claims 40-50, wherein V is selected from 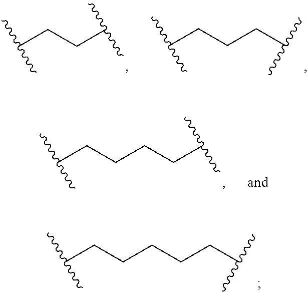
the methylene units of V are each independently unreplaced or replaced by a group selected from: —O—, —NH—, —CO—, —NHCO—,

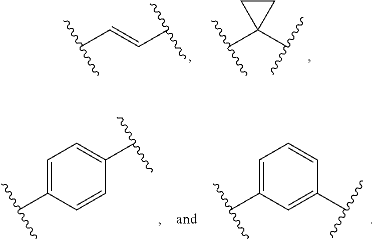

52. The ligand-drug conjugate, or the isomer thereof, or the mixture thereof, or the pharmaceutically acceptable salt thereof according to any one of claims 40-51, wherein V is selected from

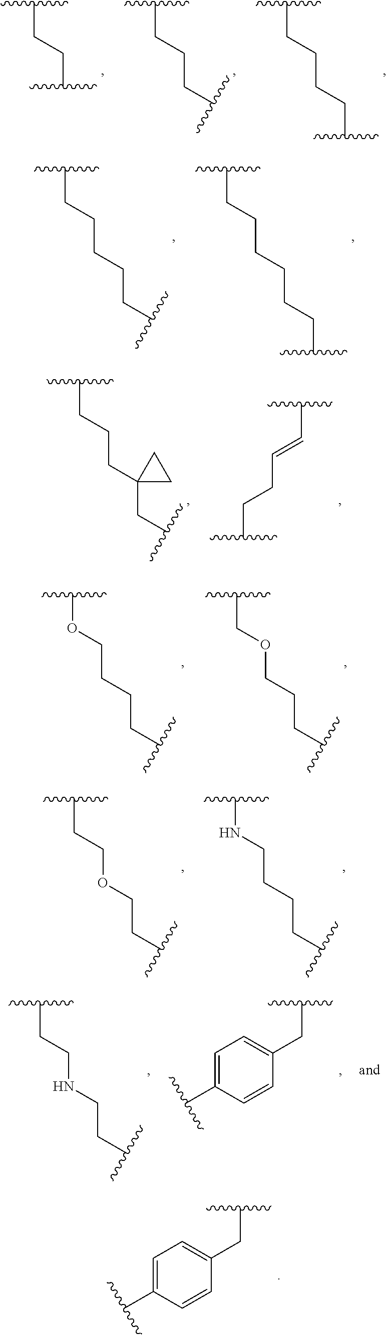

53. The ligand-drug conjugate, or the isomer thereof, or the mixture thereof, or the pharmaceutically acceptable salt thereof according to any one of claims 40-42, wherein Y is selected from —NH—, —O—, —S—,

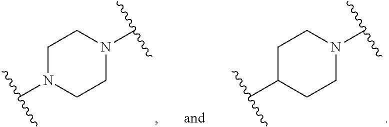 [SMILES: *N1CCN(*)CC1 ; *C1CCN(*)CC1]

54. The ligand-drug conjugate, or the isomer thereof, or the mixture thereof, or the pharmaceutically acceptable salt thereof according to any one of claims 40-53, wherein the structural unit 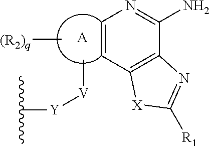
is selected from: 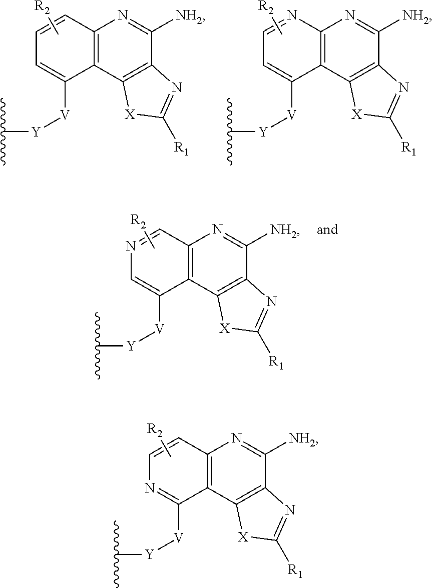
preferably selected from 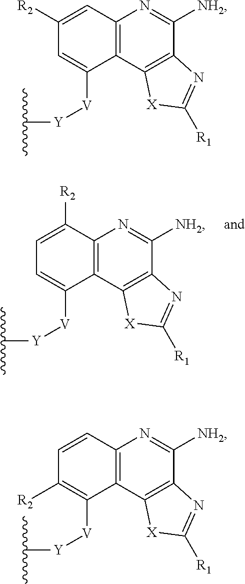
wherein
X is selected from —S— and —O—;
R₁ is as defined in any one of claims 40 and 43-45;
R₂ is as defined in claim 40, 46 or 47;
V is as defined in any one of claims 40 and 50-52;
Y is as defined in claim 40 or 53.

55. The ligand-drug conjugate, or the isomer thereof, or the mixture thereof, or the pharmaceutically acceptable salt thereof according to any one of claims 40-42, wherein the linker unit L is -Tr-L₃-L₂-L_{1c}-, wherein
Tr is selected from 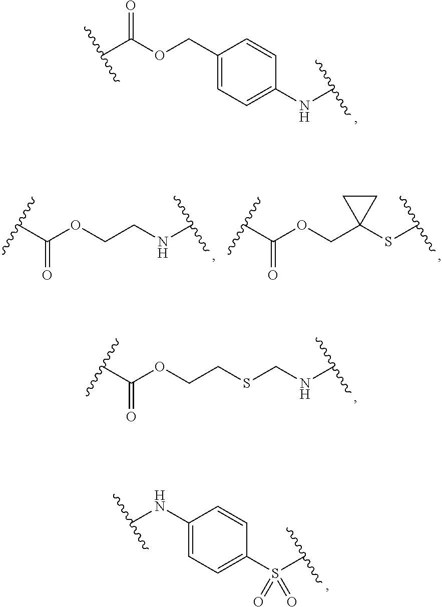 [SMILES: *Nc1ccc(COC(*)=O)cc1 ; *NCCOC(*)=O ; *SC1(COC(*)=O)CC1 ; *NCSCCOC(*)=O ; *Nc1ccc(S(*)(=O)=O)cc1]
and a single bond;
L_{1c} is selected from 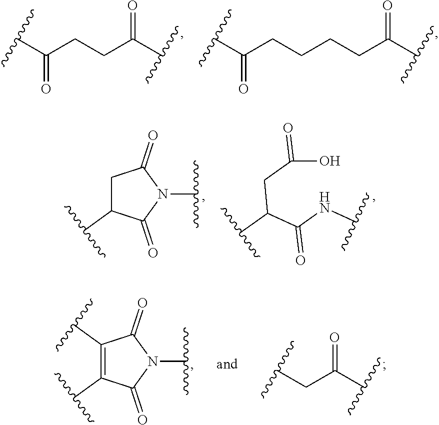 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 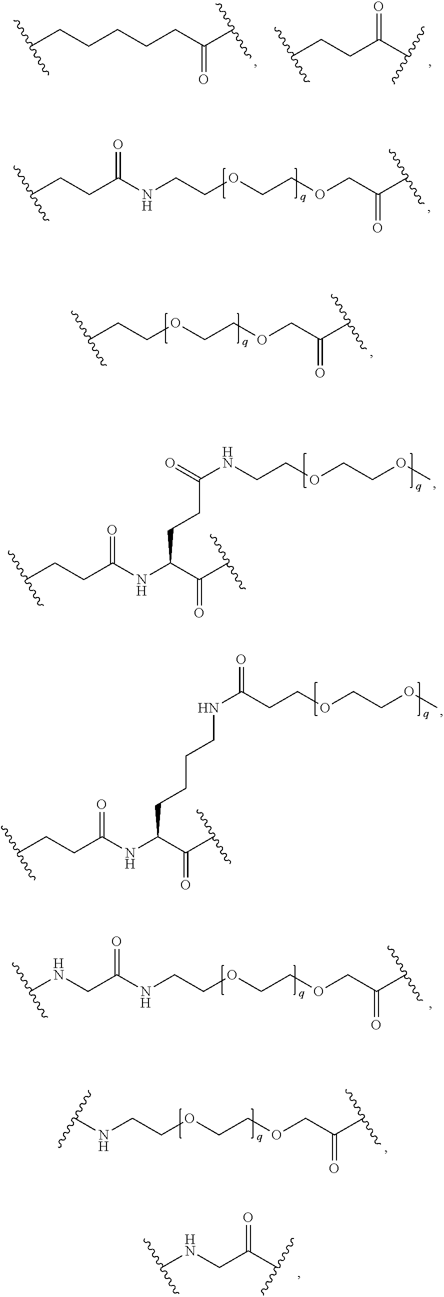
and a single bond, wherein q is an integer from 1 to 20;
L₃ is selected from a single bond or a peptide residue consisting of 1-4 amino acids, wherein the peptide residue is a peptide residue formed by an amino acid selected from the group consisting of: valine, lysine, cysteine, alanine, glycine, glutamic acid, phenylalanine, and serine.

56. The ligand-drug conjugate, or the isomer thereof, or the mixture thereof, or the pharmaceutically acceptable salt thereof according to claim 55, wherein L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

57. The ligand-drug conjugate, or the isomer thereof, or the mixture thereof, or the pharmaceutically acceptable salt thereof according to any one of claims 40-56, wherein the linker unit L is -L₃-L₂-L_{1c}-, wherein
L_{1c} is selected from 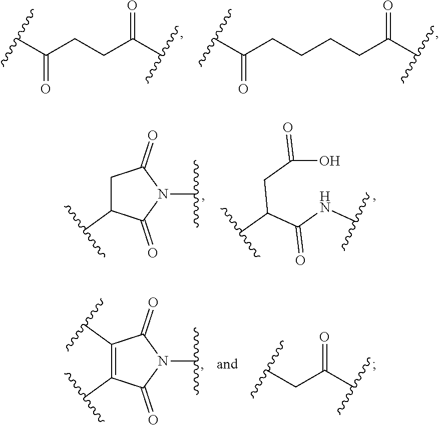 [SMILES: *C(=O)CCC(*)=O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 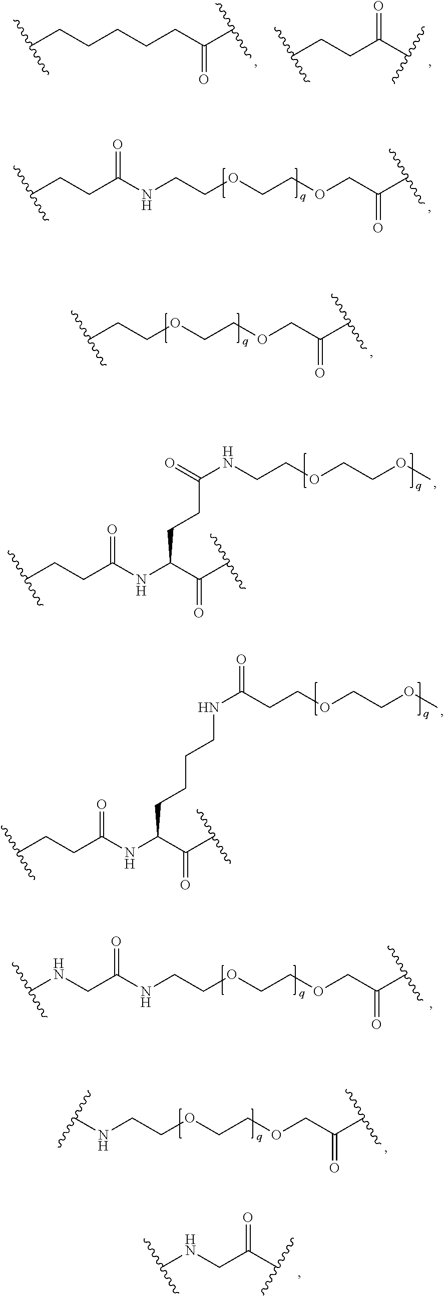
and a single bond, wherein q is an integer from 1 to 20;
L₃ is selected from a single bond or the following peptide residues: -valine-cysteine-, -valine-citrulline-, valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

58. The ligand-drug conjugate, or the isomer thereof, or the mixture thereof, or the pharmaceutically acceptable salt thereof according to any one of claims 40-57, wherein the linker unit L is -L₃-L₂-L_{1c}- or -Tr-L₃-L₂-L_{1c}-, wherein the L₃ end or the Tr end is connected to the nitrogen atom of the Z group, and the L_{1c} end is connected to the ligand.

59. The ligand-drug conjugate, or the isomer thereof, or the mixture thereof, or the pharmaceutically acceptable salt thereof according to any one of claims 40-58, wherein the linker unit L is selected from:

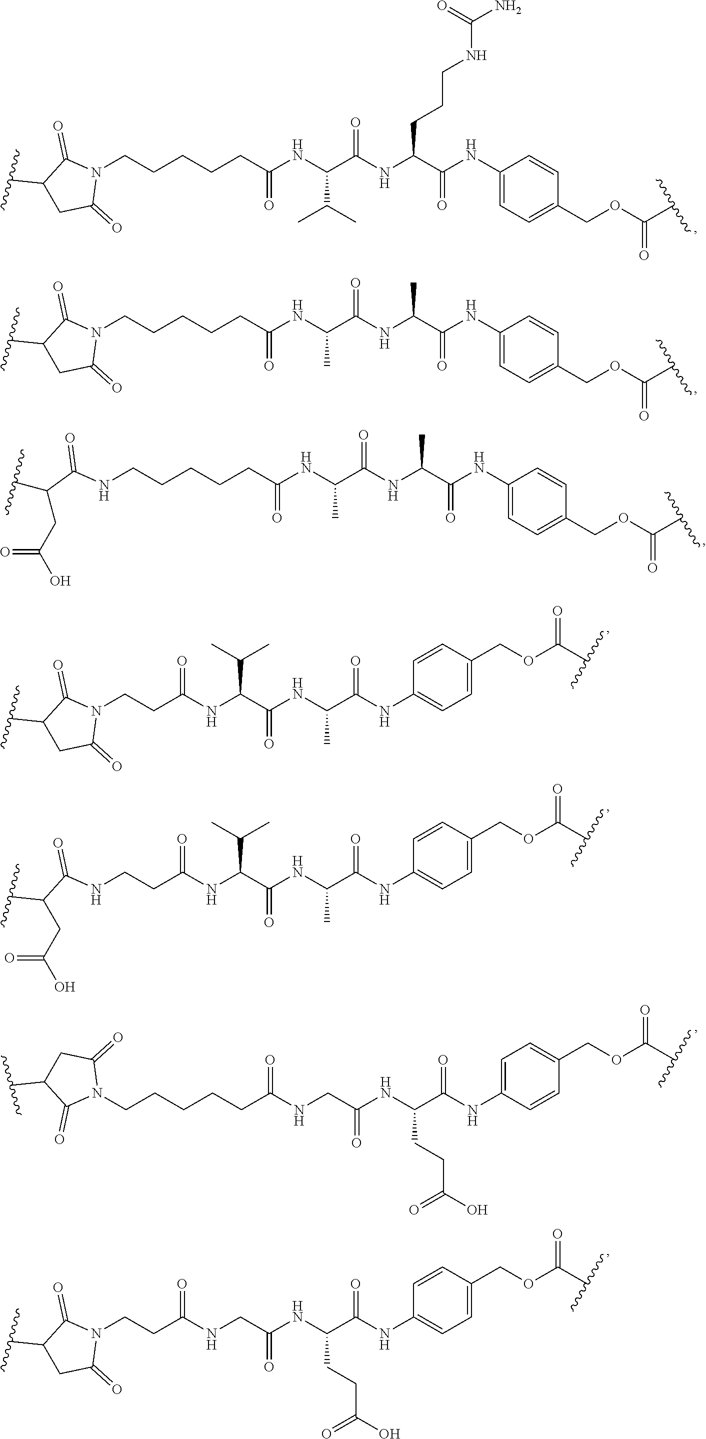

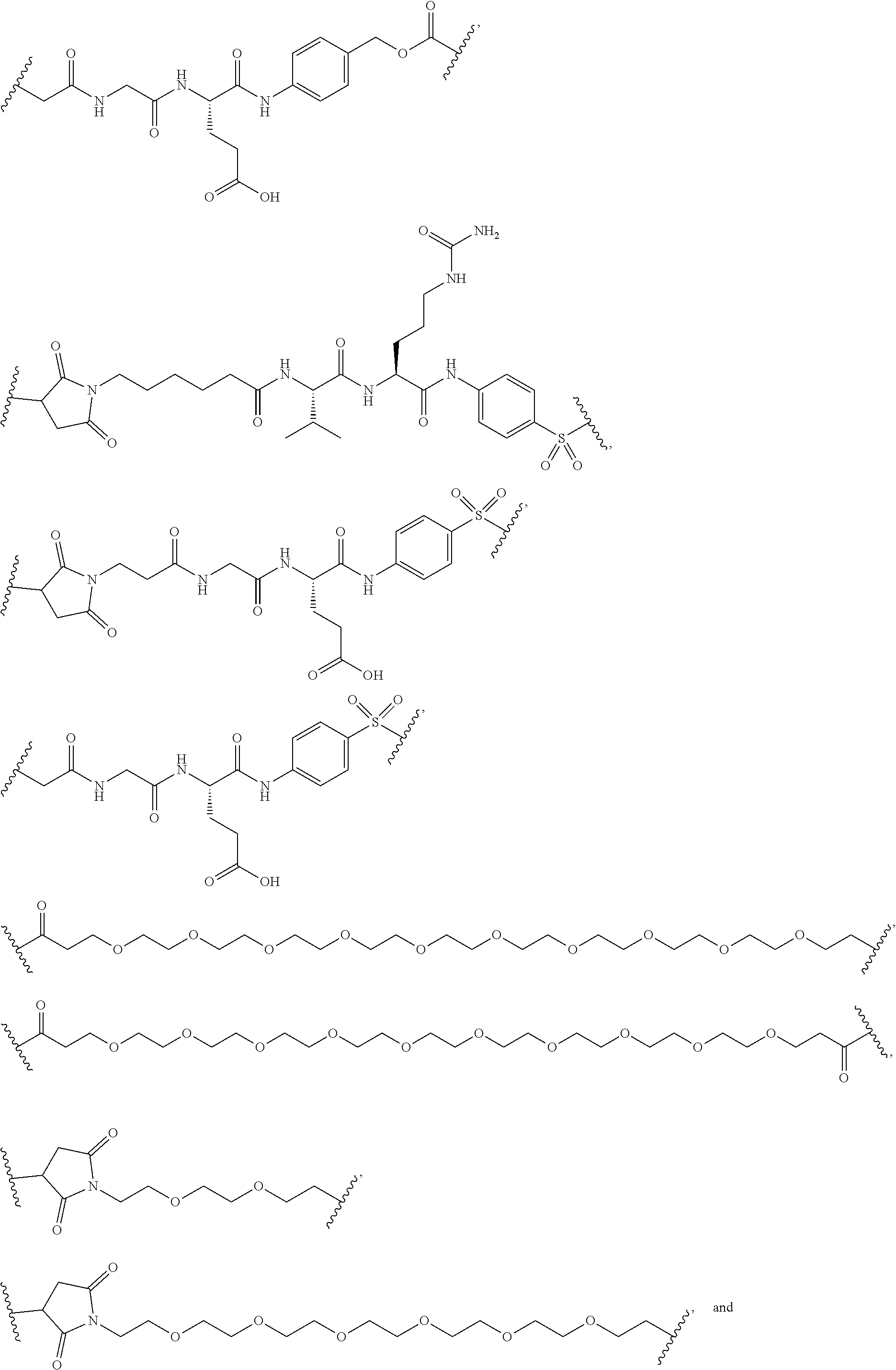

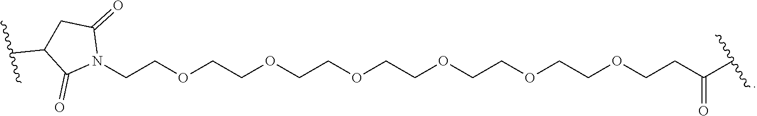 [SMILES: *C(=O)CCOCCOCCOCCOCCOCCOCCN1C(=O)CC(*)C1=O]

60. The ligand-drug conjugate, or the isomer thereof, or the mixture thereof, or the pharmaceutically acceptable salt thereof according to any one of claims 40-59, wherein the ligand-drug conjugate is represented by formulas (III-2C-1) and (II-2C-2): 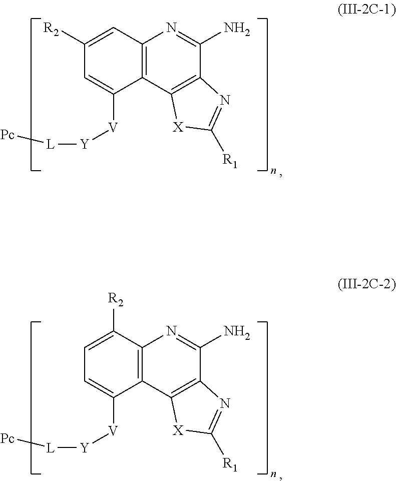
wherein
X is selected from —S— and —O—;
R₁ is selected from C₁-C₆ alkyl optionally substituted with 1, 2, or 3 R₁₋₁, each R₁₋₁ being independently selected from: hydrogen, chlorine, bromine, iodine, hydroxy, amino, C₁-C₃ alkyl, and C₃-C₆ cycloalkyl; the methylene units of R₁ are each independently unreplaced or replaced by a group selected from: —O—, —N(CH₃)—, and —NH—;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂₋₁; each R₂₋₁ is independently selected from: hydrogen, halogen, hydroxy, and amino;
V is selected from C₁-C₈alkylene, preferably C₁-C₆ alkylene, wherein one or more carbon atoms in the C₁-C₈ alkylene and the C₁-C₆ alkylene can be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
Y is —N(R_{Y1})—;
R_{Y1} is selected from hydrogen and C₁-C₆ alkyl;
L is -L₃-L₂-L_{1c}- or -Tr-L₃-L₂-L_{1c}-;
Tr is selected from: 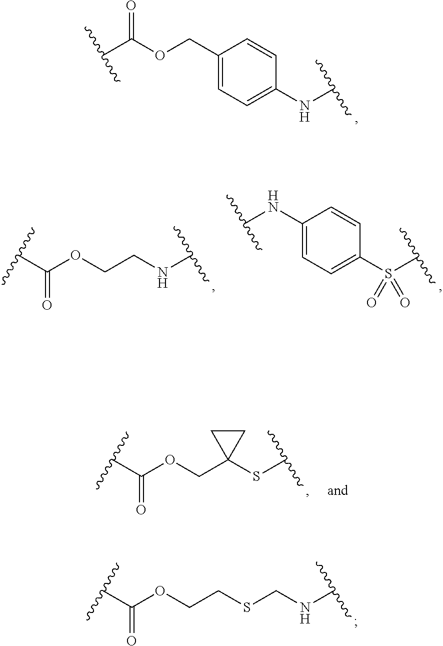 [SMILES: *Nc1ccc(COC(*)=O)cc1 ; *NCCOC(*)=O ; *Nc1ccc(S(*)(=O)=O)cc1 ; *SC1(COC(*)=O)CC1 ; *NCSCCOC(*)=O]
L_{1c} is selected from 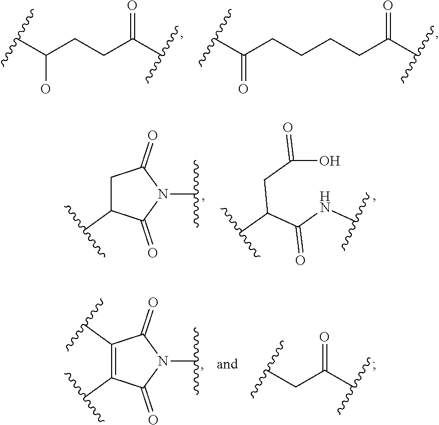 [SMILES: *C(=O)CCC(*)O ; *C(=O)CCCCC(*)=O ; *C1CC(=O)N(*)C1=O ; *NC(=O)C(*)CC(=O)O ; *C1=C(*)C(=O)N(*)C1=O ; *CC(*)=O]
L₂ is selected from 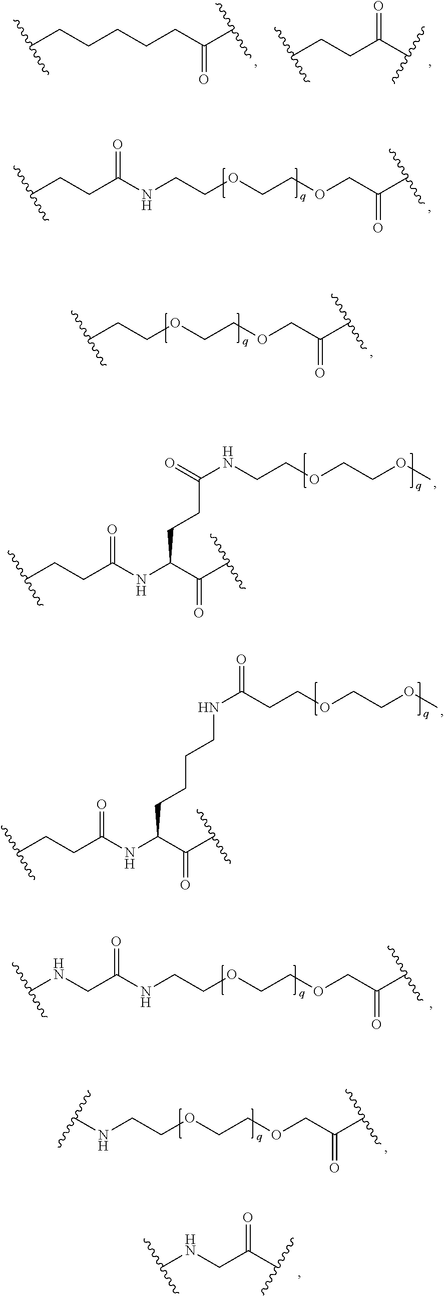
and a single bond, wherein q is an integer from 1 to 20;
L₃ is selected from the following peptide residues: -valine-cysteine-, -valine-citrulline-, -valine-lysine-, -valine-alanine-, -alanine-alanine-, -glycine-glycine-, -glycine-glutamic acid-, -glycine-glutamine-, -glycine-aspartic acid-, -glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-;
n is an integer or a decimal from 1 to 10;
Pc is a ligand.

61. The ligand-drug conjugate, or the isomer thereof, or the mixture thereof, or the pharmaceutically acceptable salt thereof according to any one of claims 40-60, wherein the ligand-drug conjugate is selected from the following structural formulas: 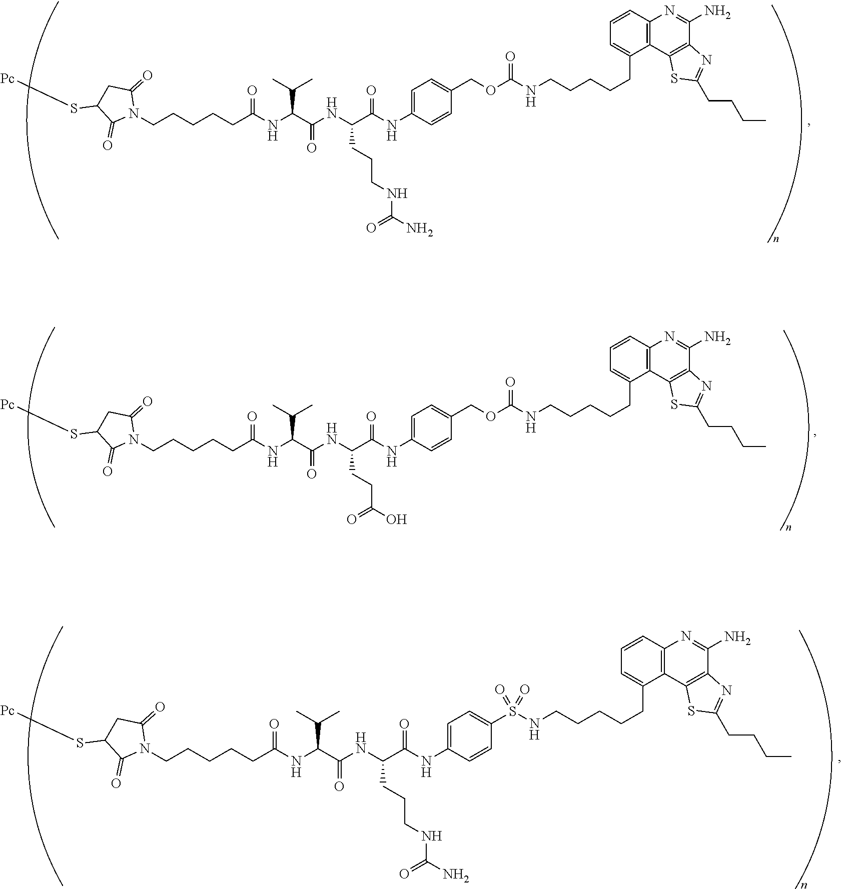 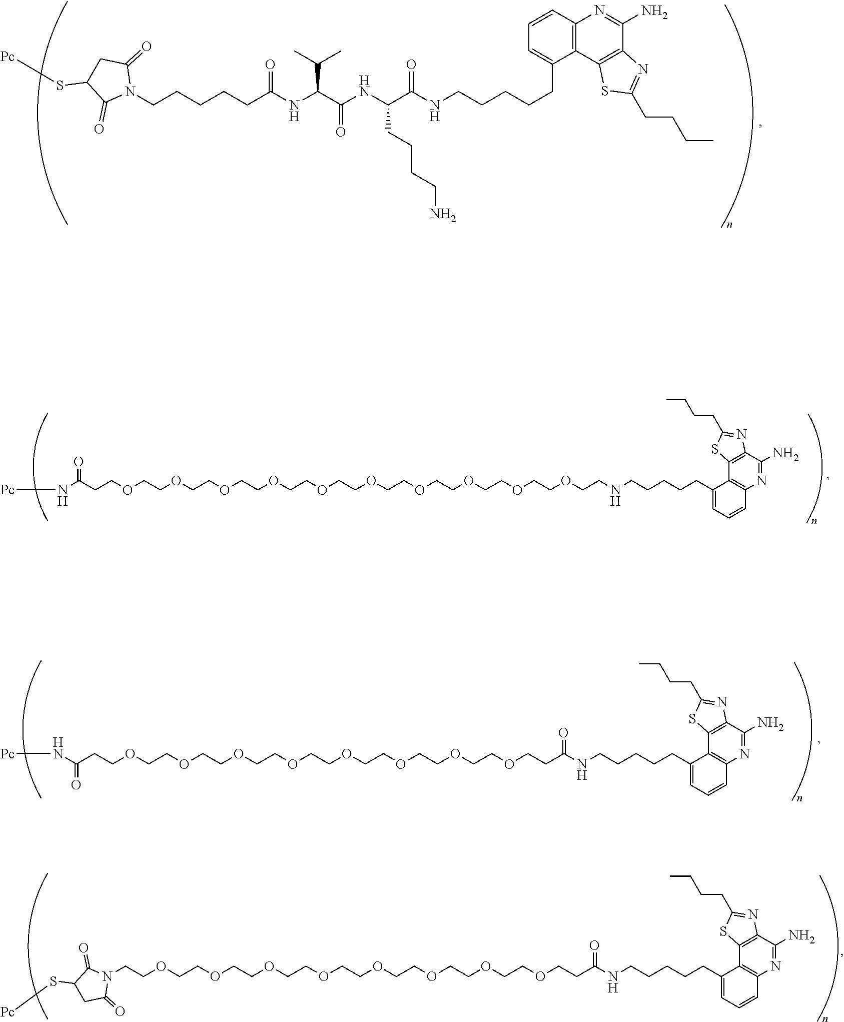 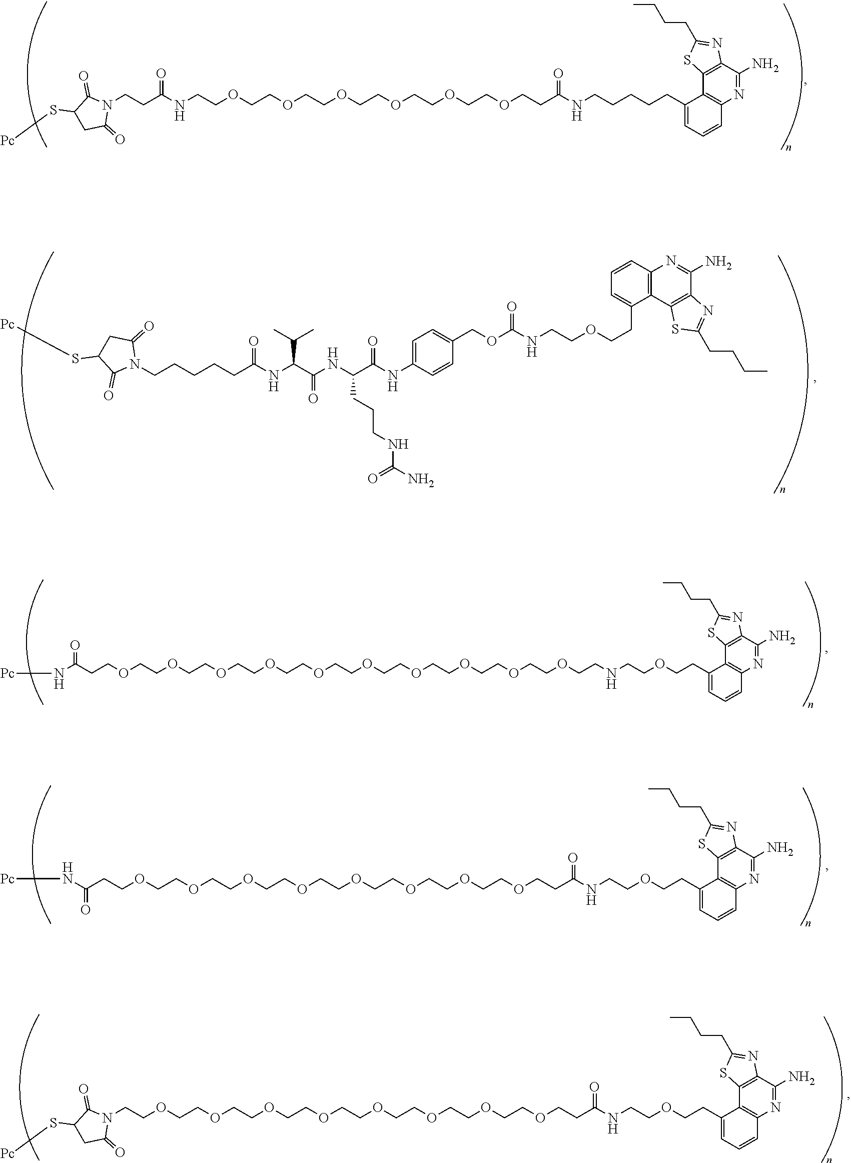 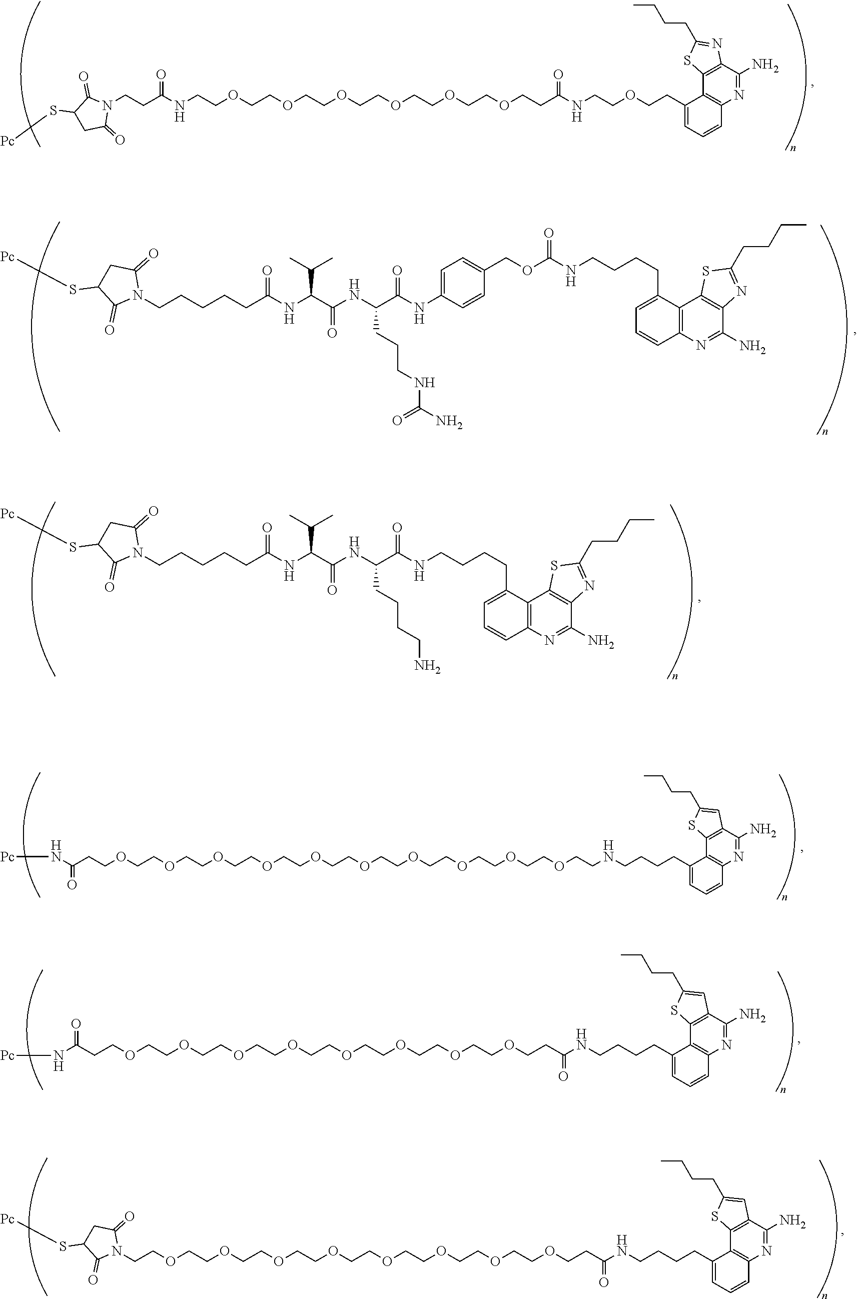 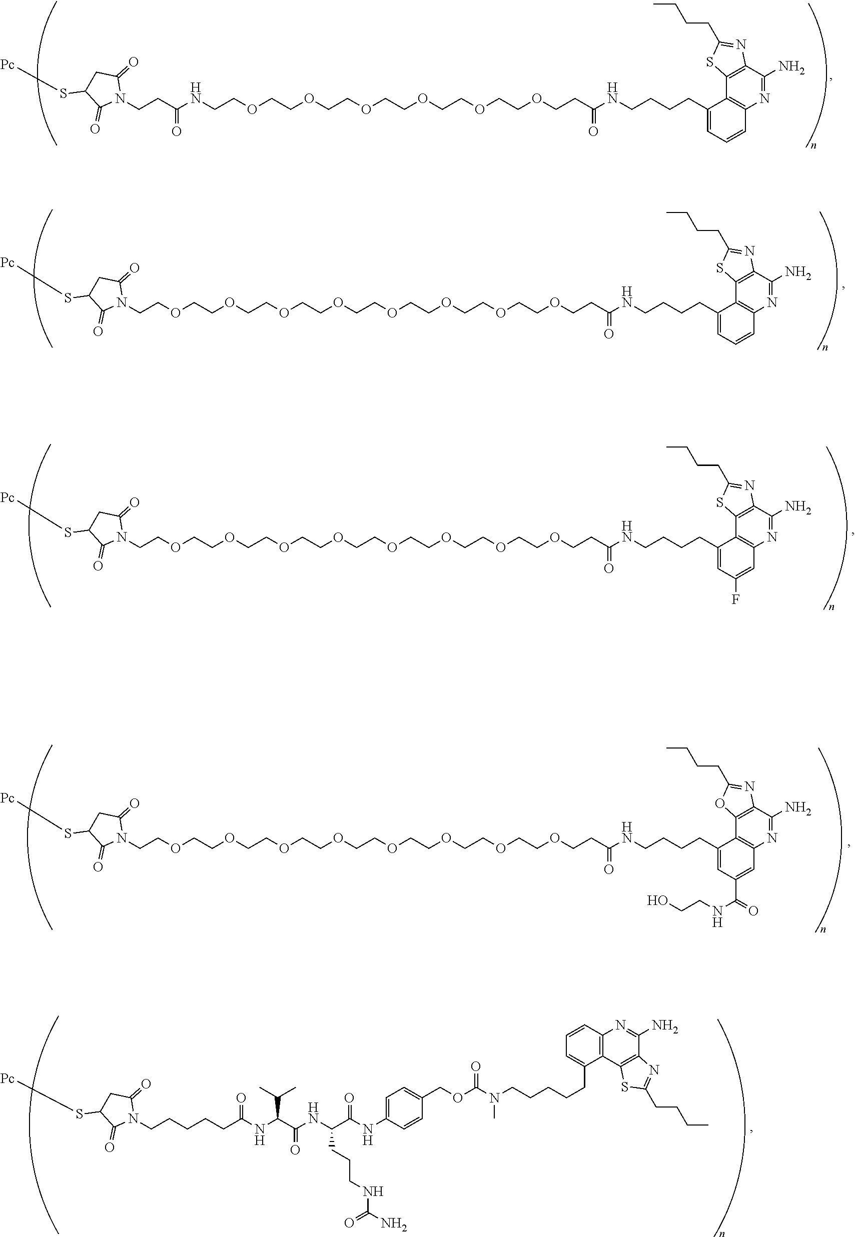 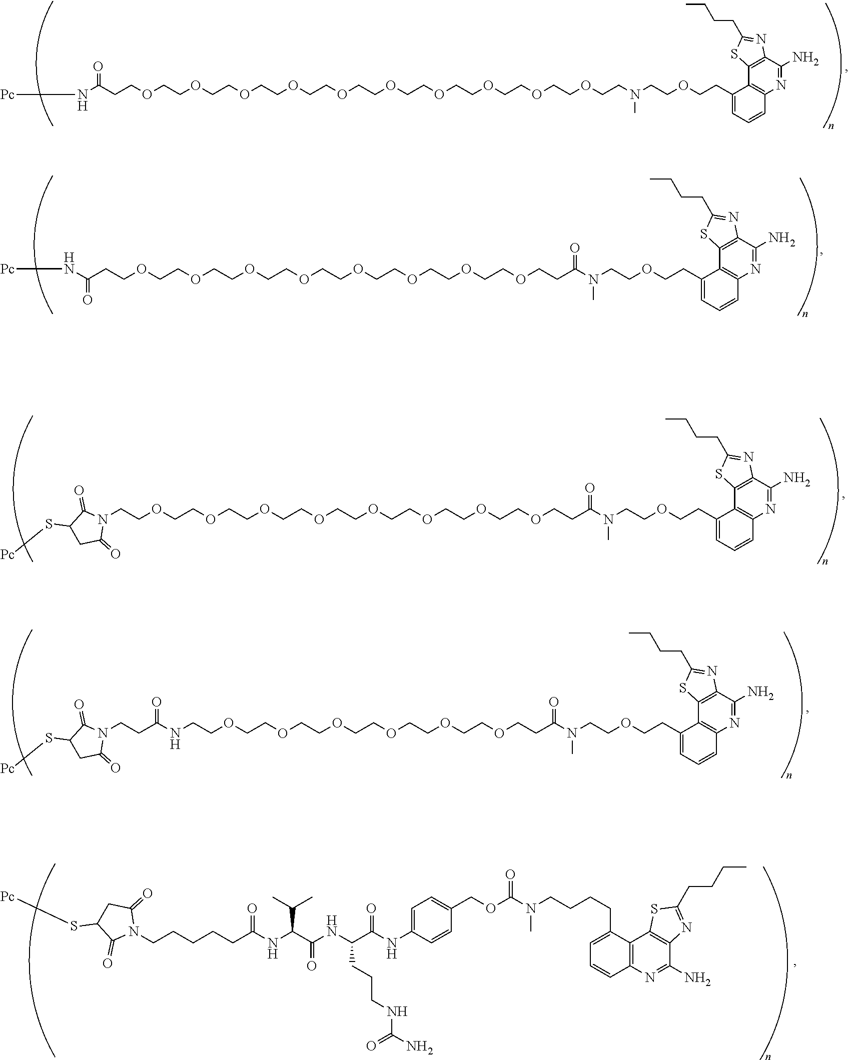 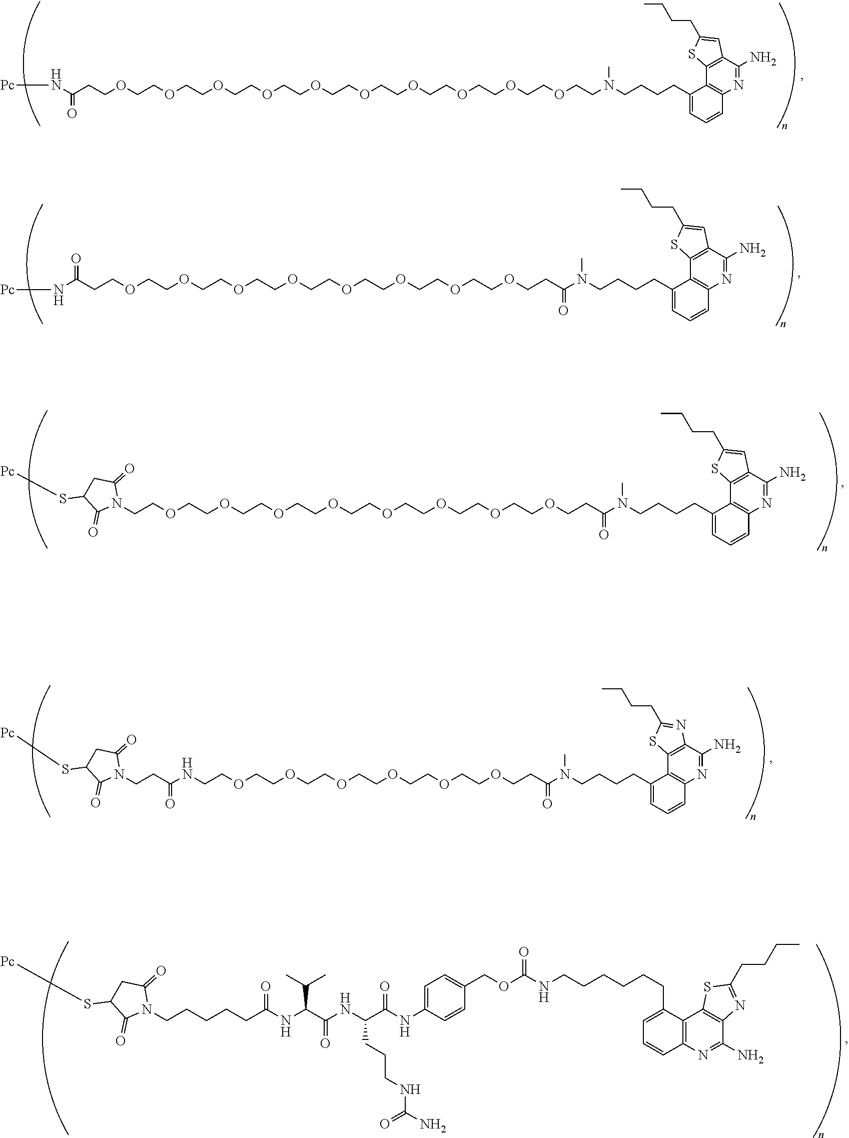 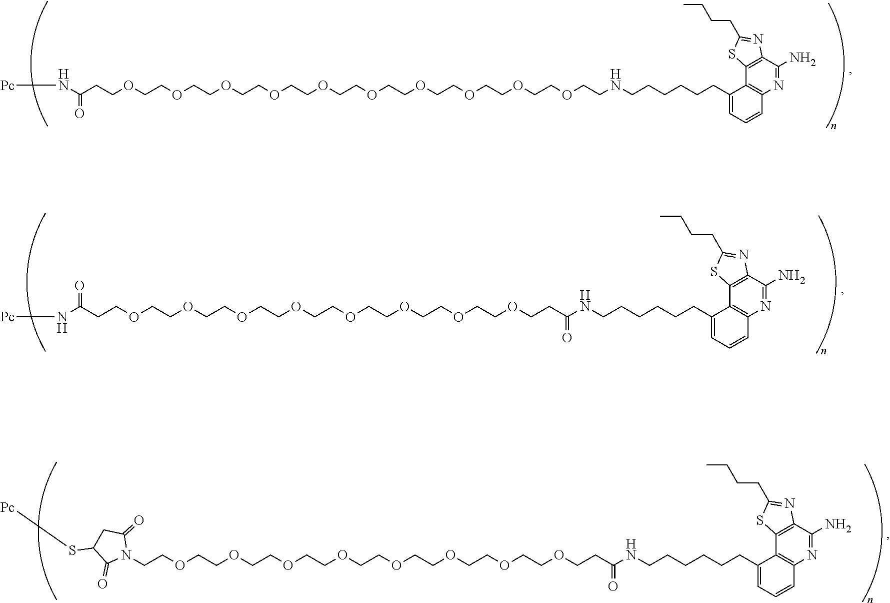 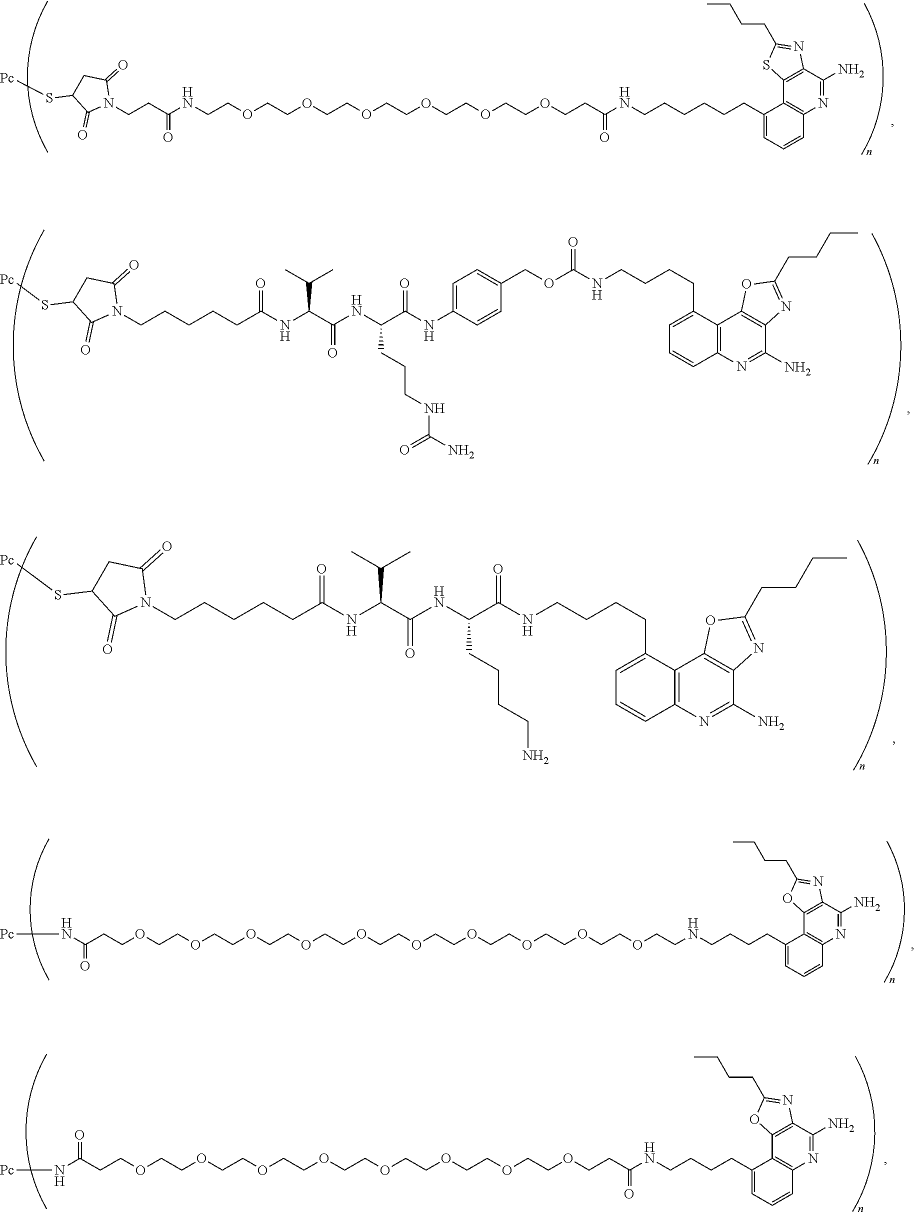 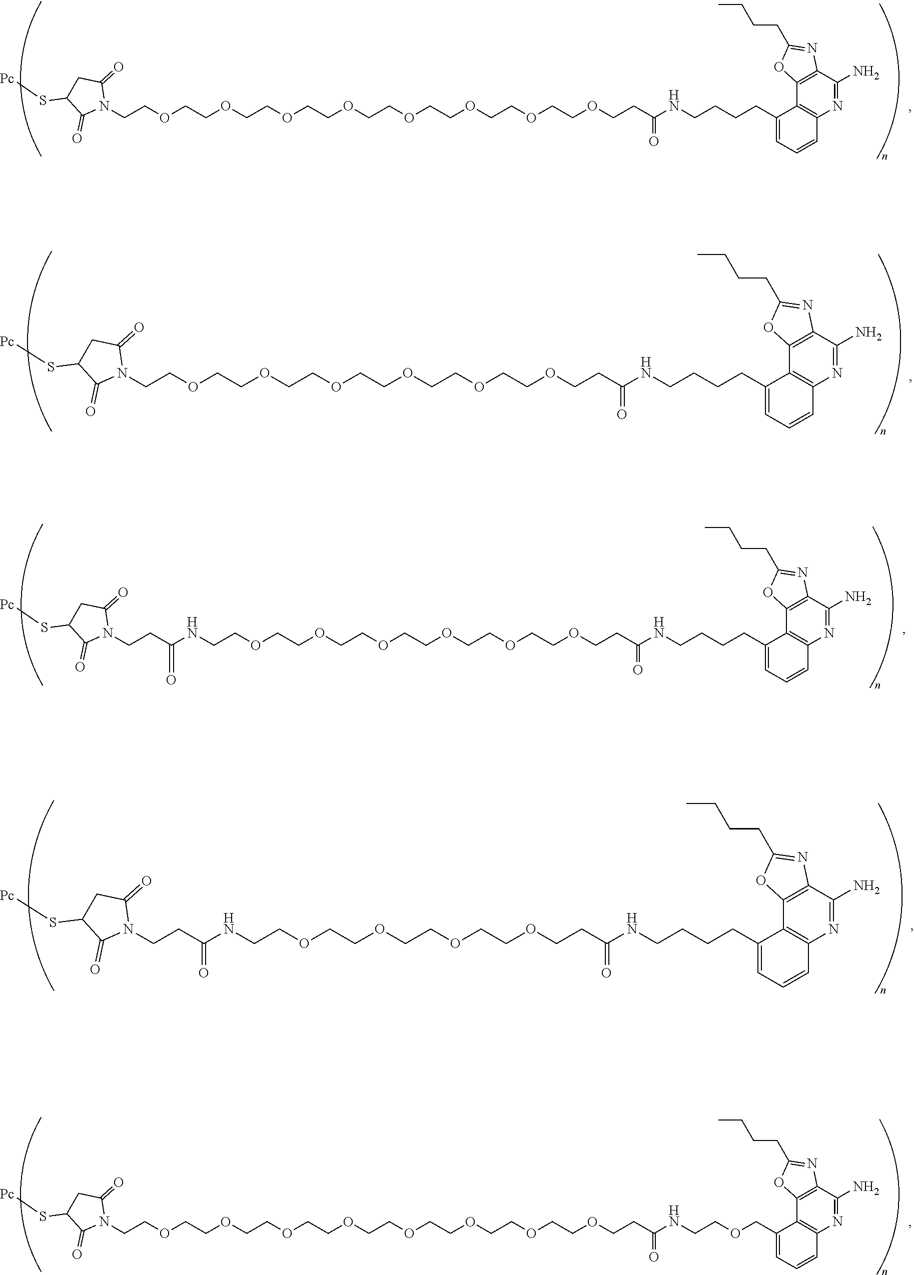 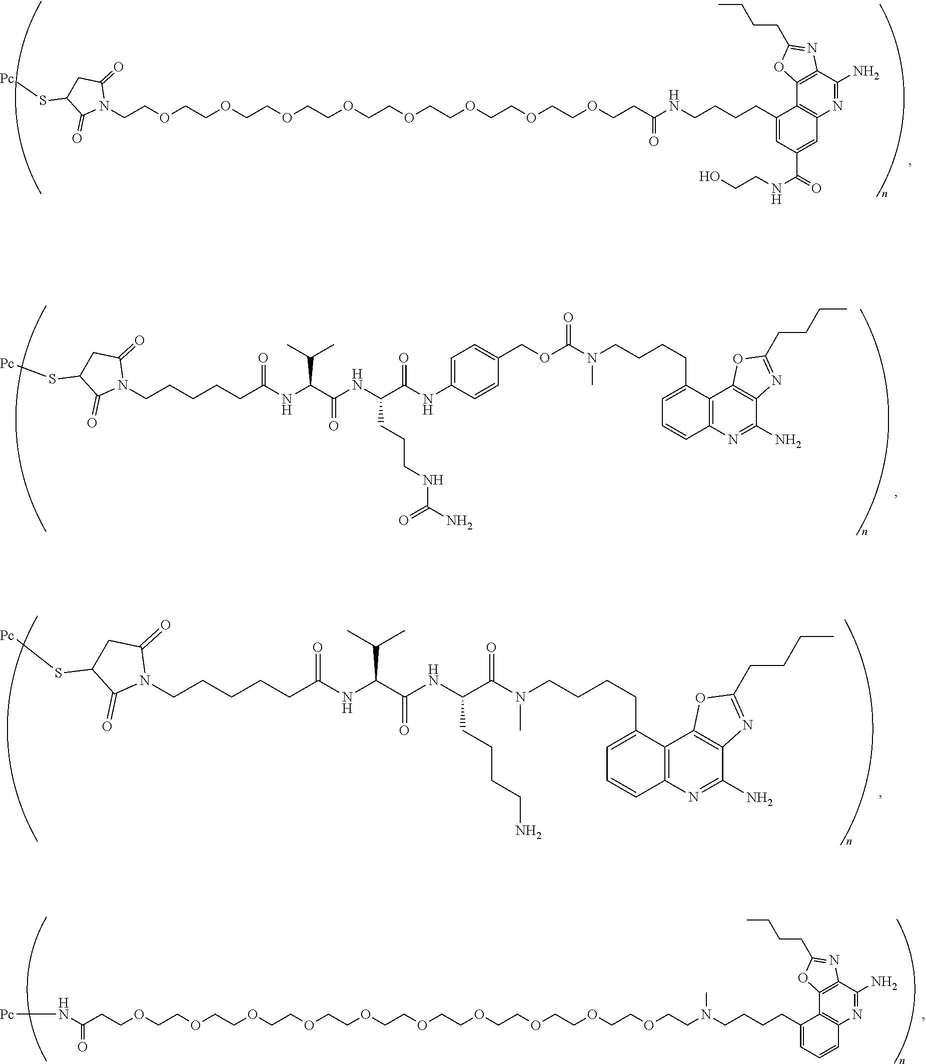 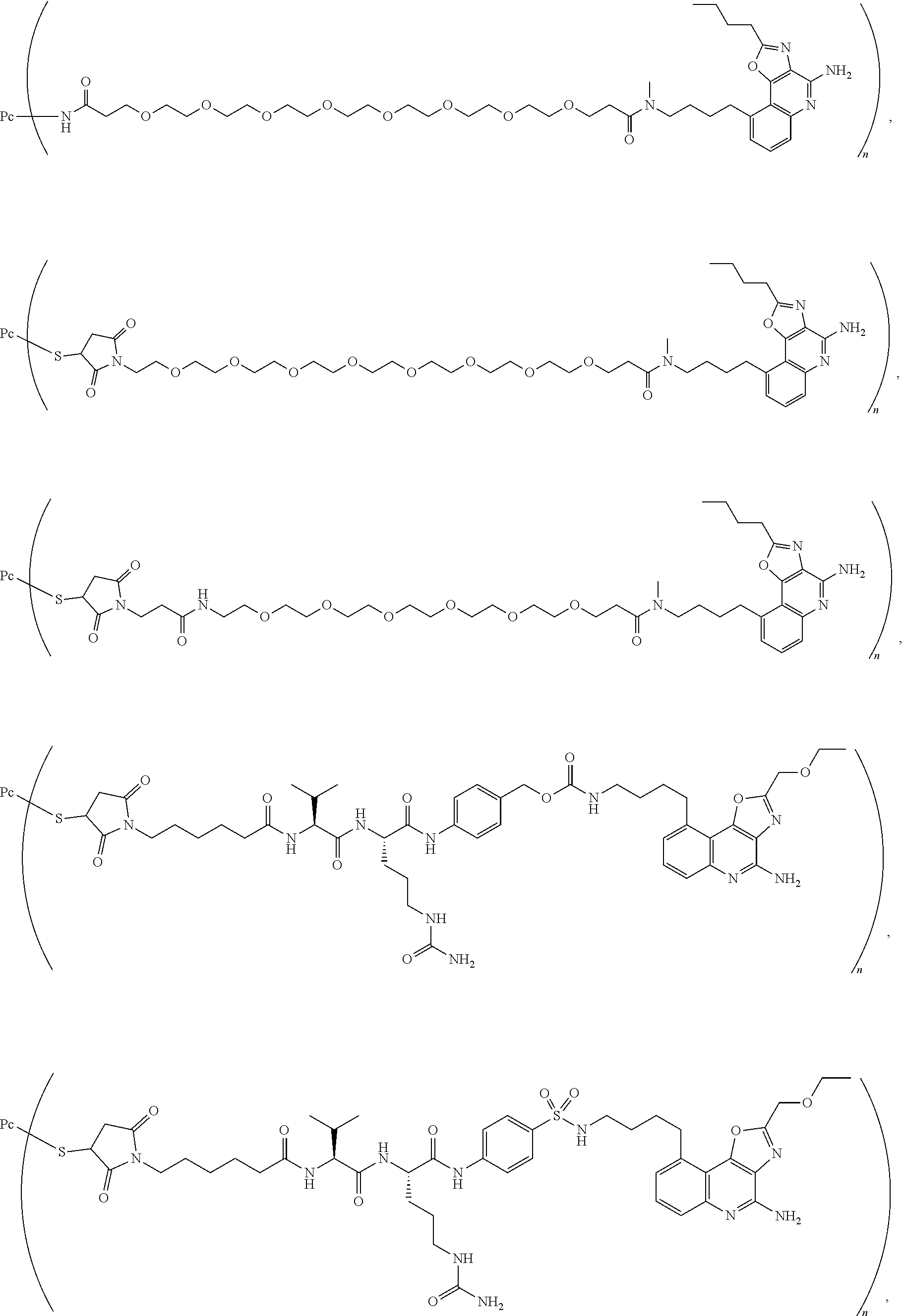 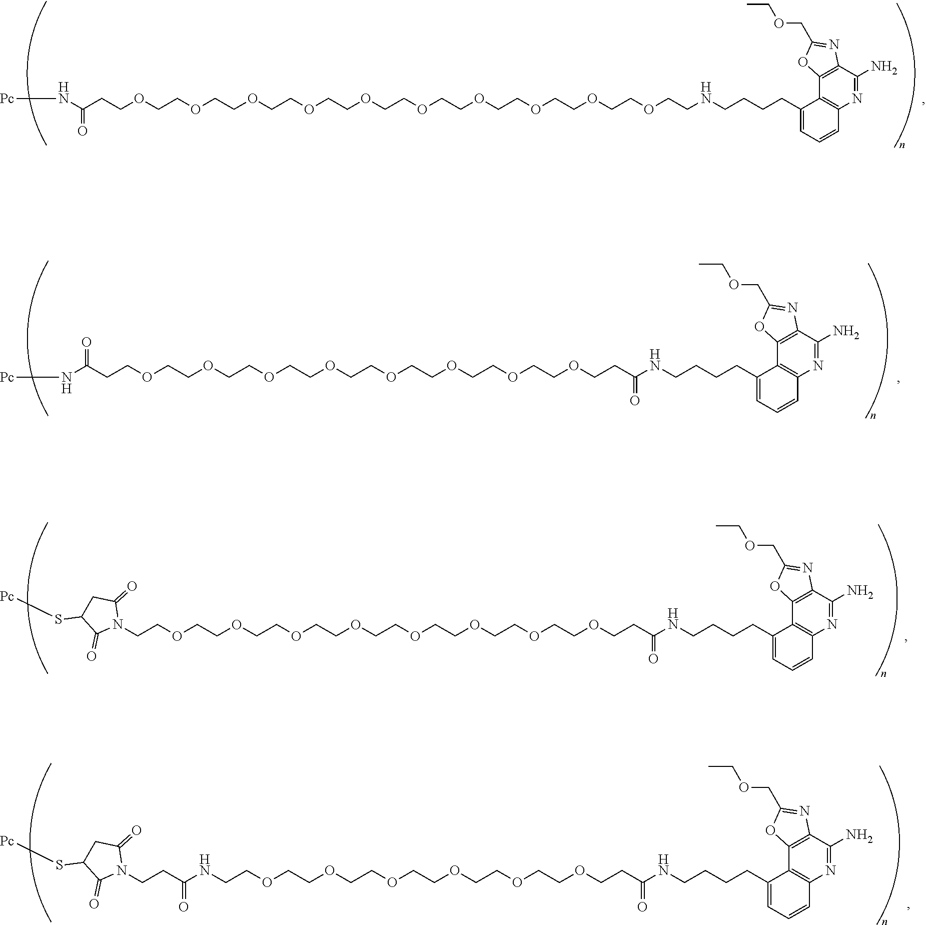 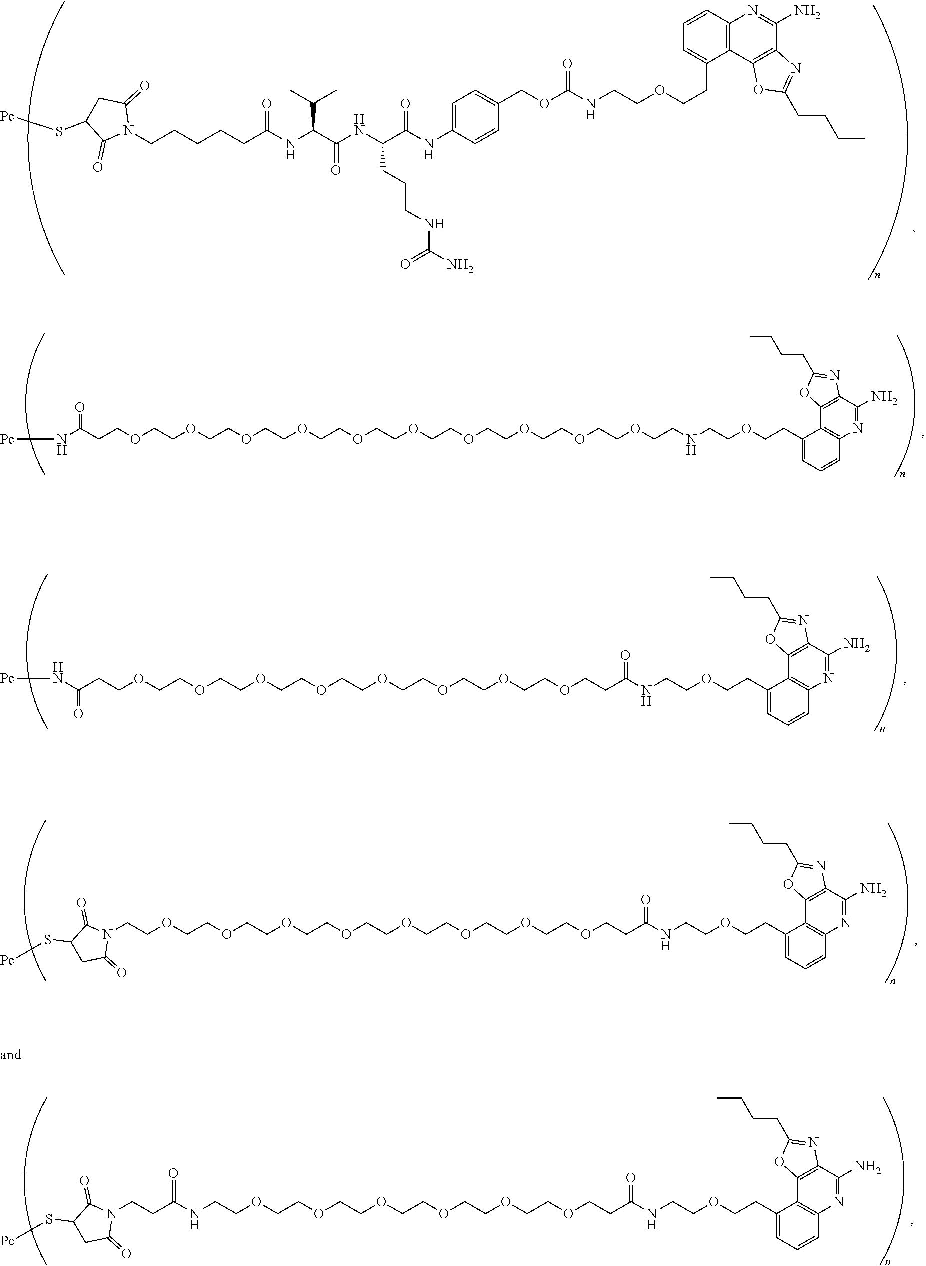
wherein
n is an integer or a decimal from 1 to 10;
Pc is a ligand.

62. The ligand-drug conjugate, or the isomer thereof, or the mixture thereof, or the pharmaceutically acceptable salt thereof according to any one of claims 40-61, wherein n is an integer or a decimal from 2 to 8.

63. The ligand-drug conjugate, or the isomer thereof, or the mixture thereof, or the pharmaceutically acceptable salt thereof according to any one of claims 40-62, wherein Pc is an antibody or an antigen-binding fragment thereof.

64. The ligand-drug conjugate, or the isomer thereof, or the mixture thereof, or the pharmaceutically acceptable salt thereof according to claim 63, wherein Pc targets GPC3, Trop2, or HER2; preferably, the antibody is selected from: trastuzumab, pertuzumab, sacituzumab, and codrituzumab.

65. The ligand-drug conjugate, or the isomer thereof, or the mixture thereof, or the pharmaceutically acceptable salt thereof according to any one of claims 40-64, wherein the ligand-drug conjugate is selected from the following structural formulas: 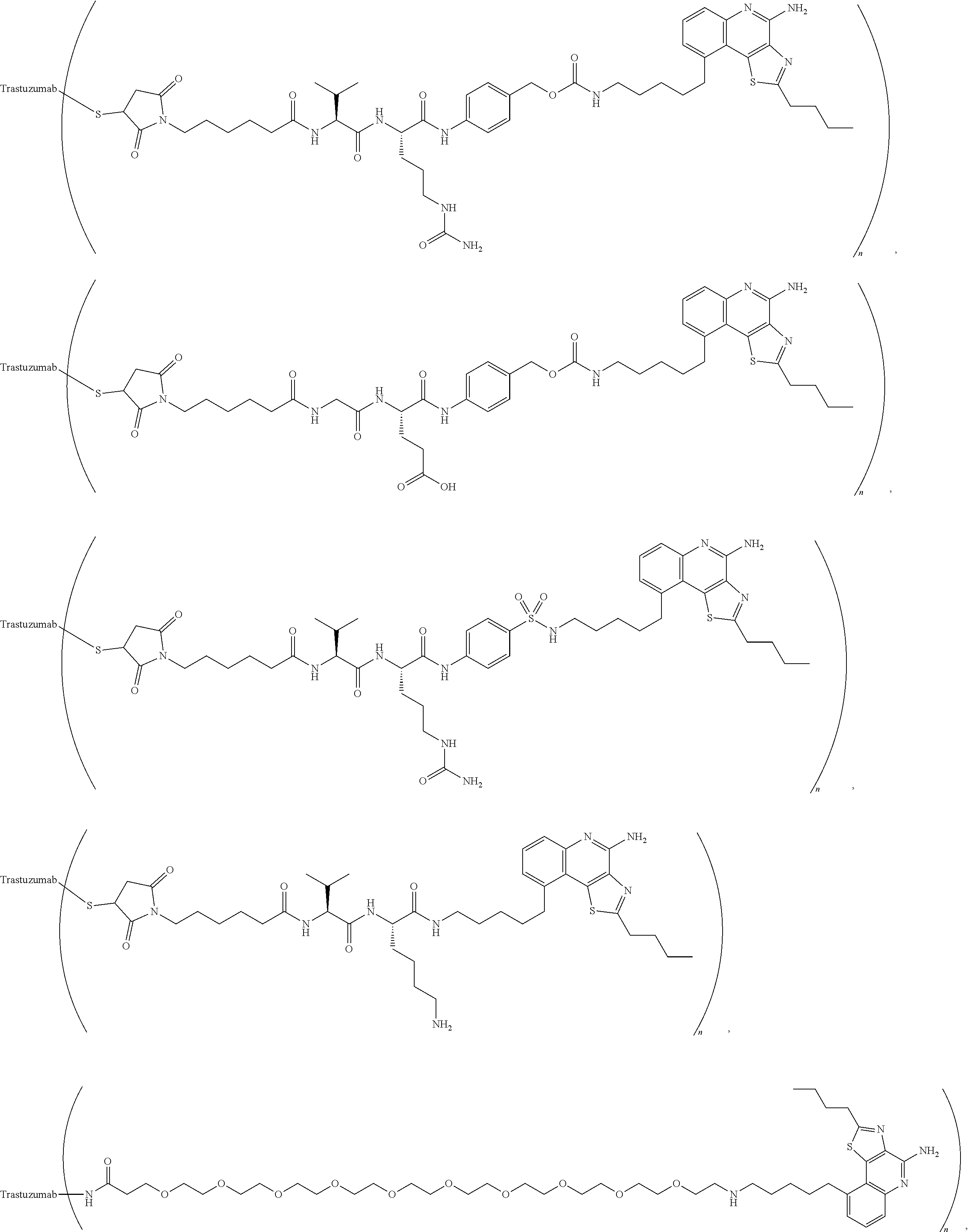 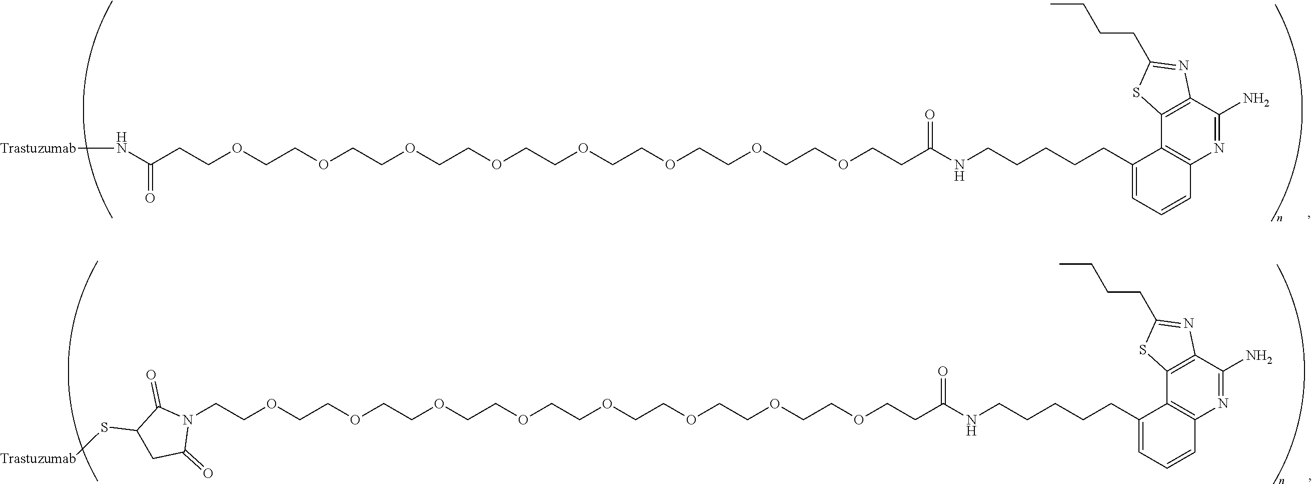 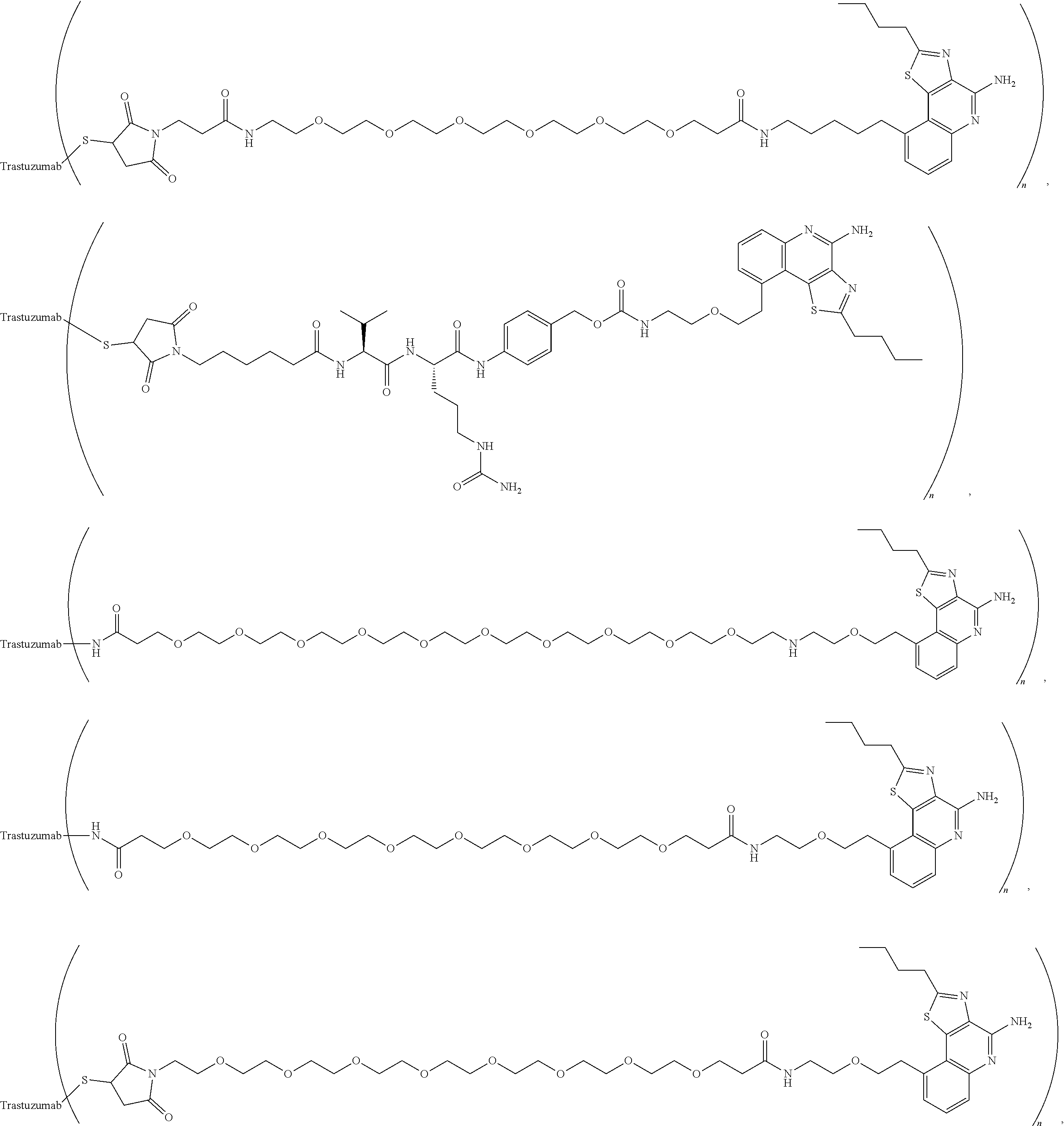 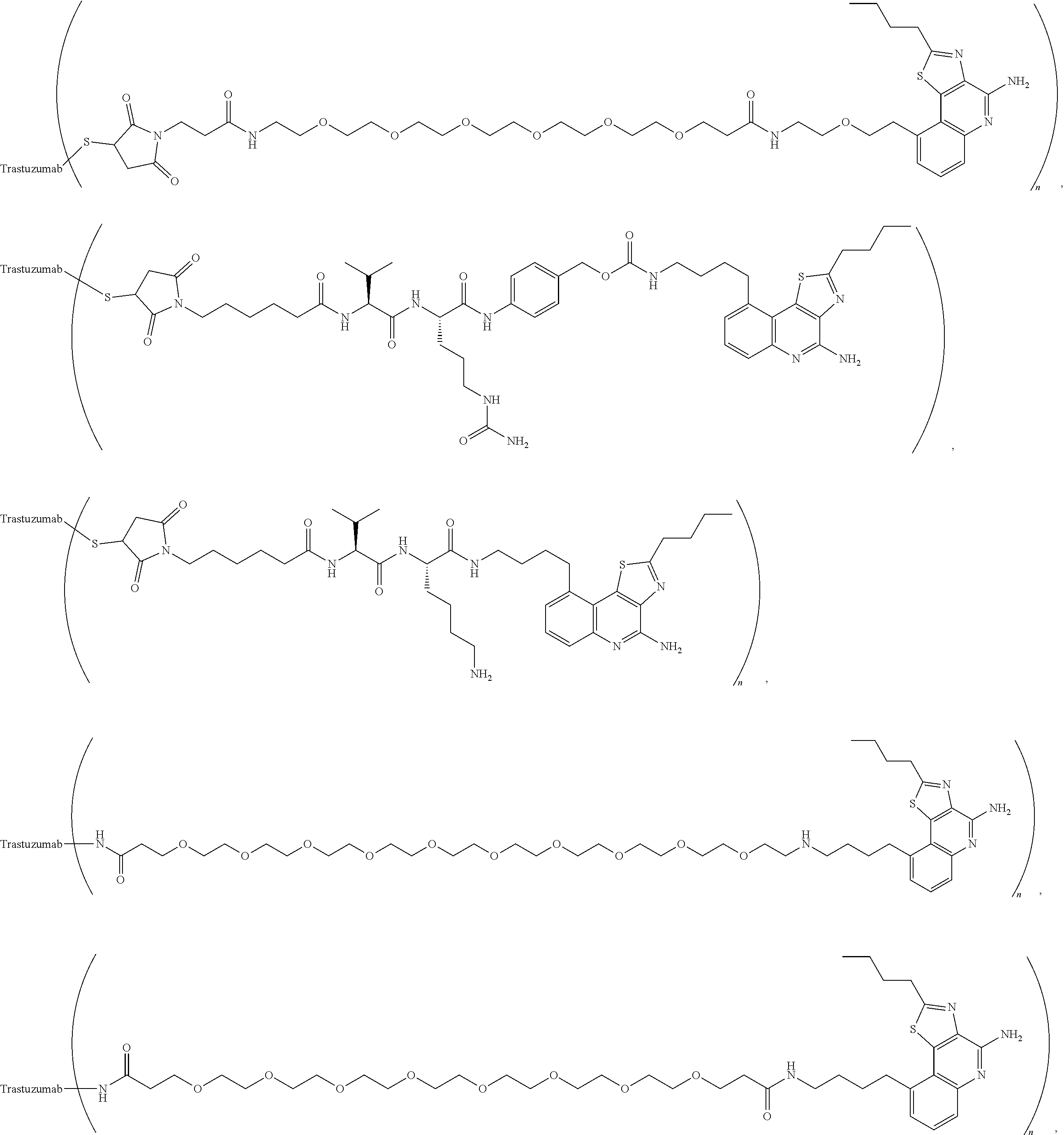 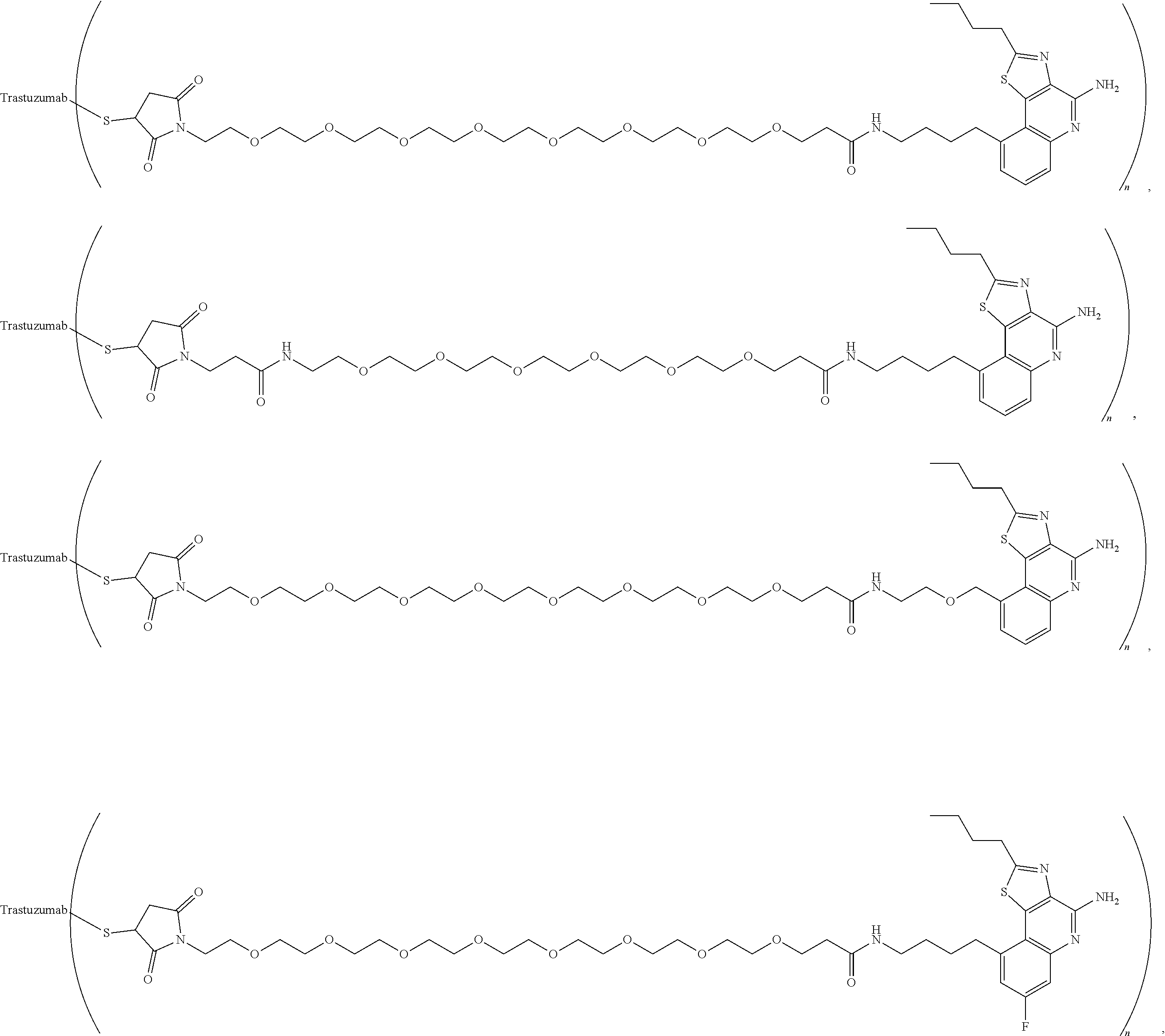 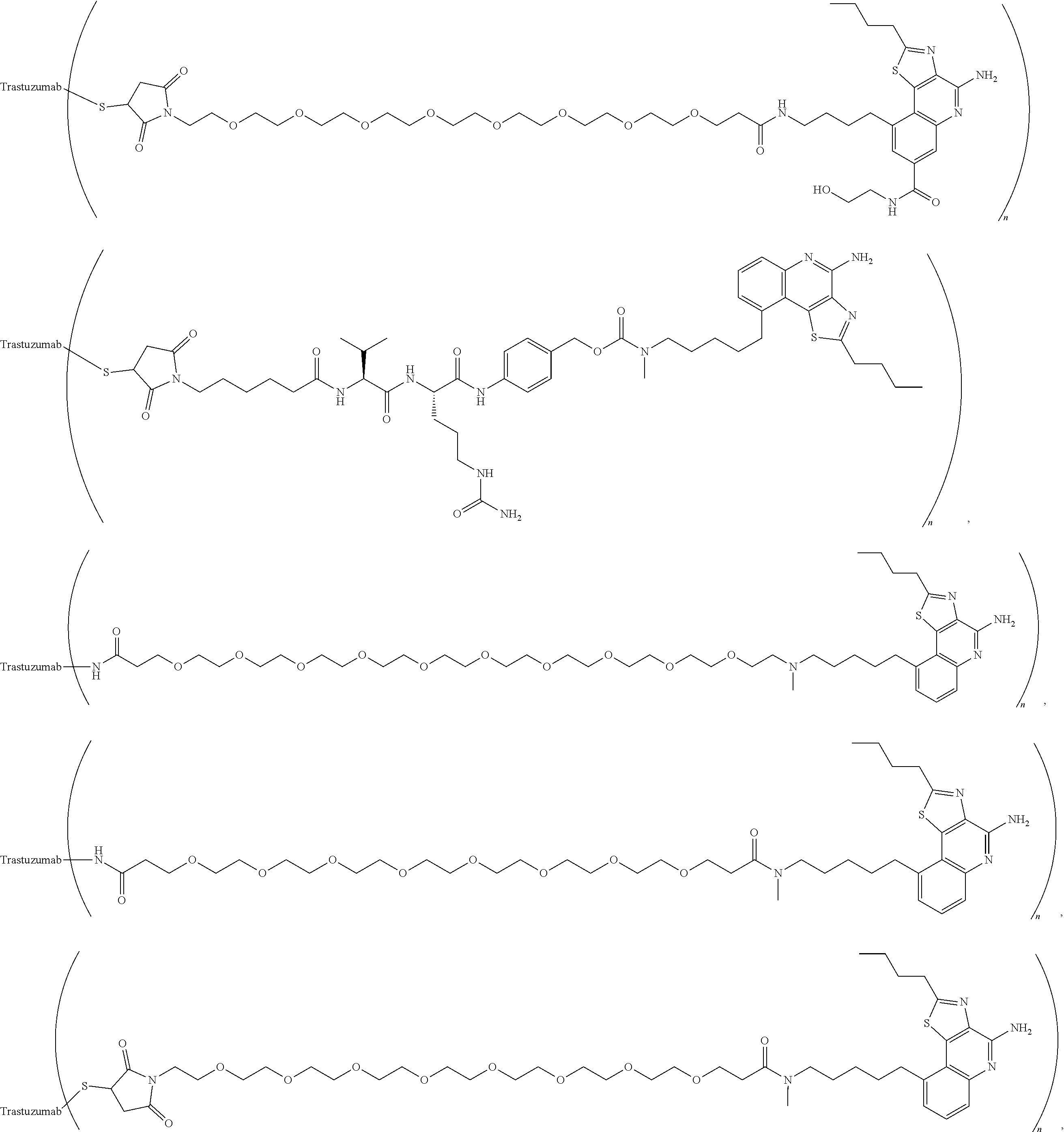 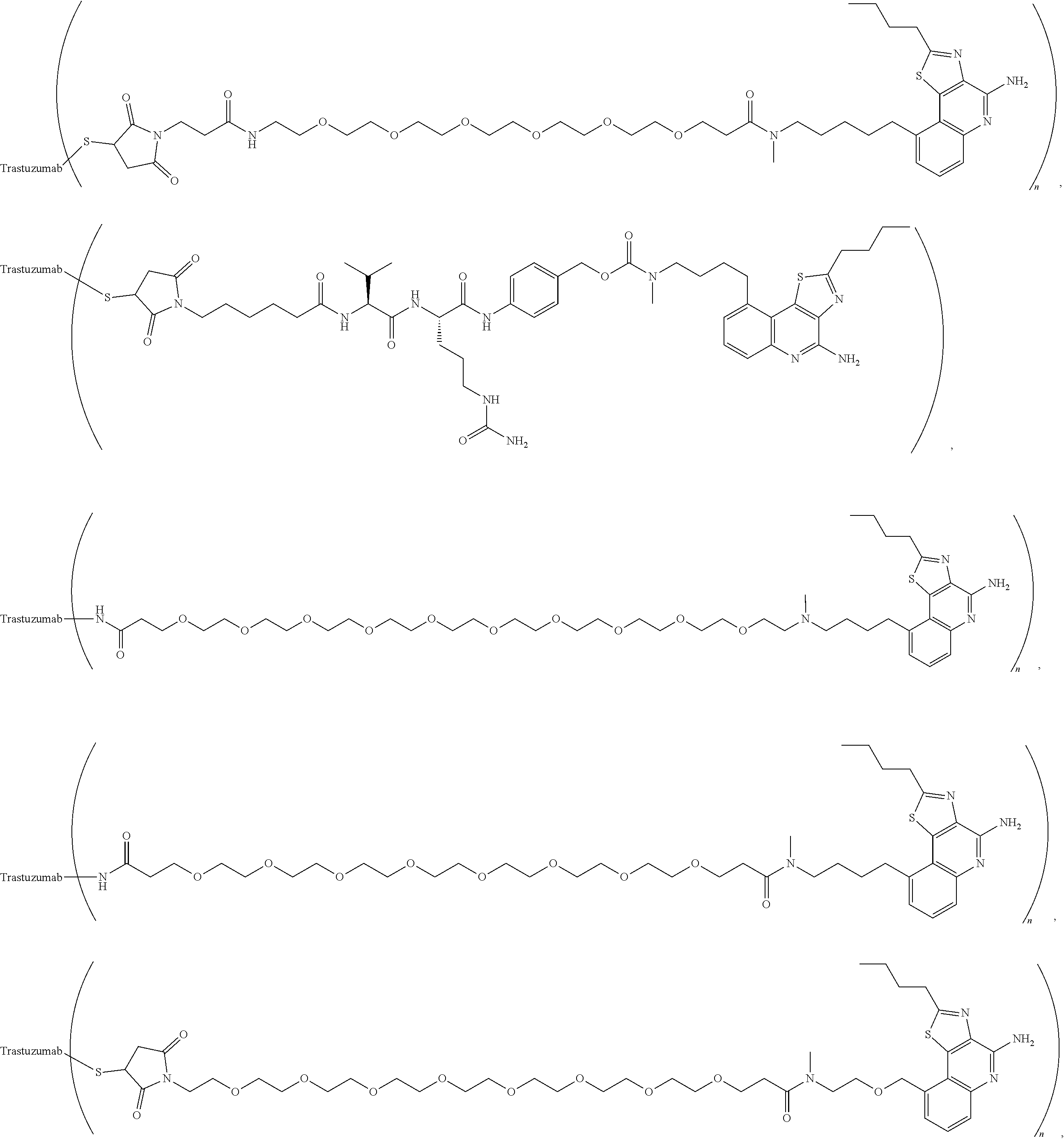 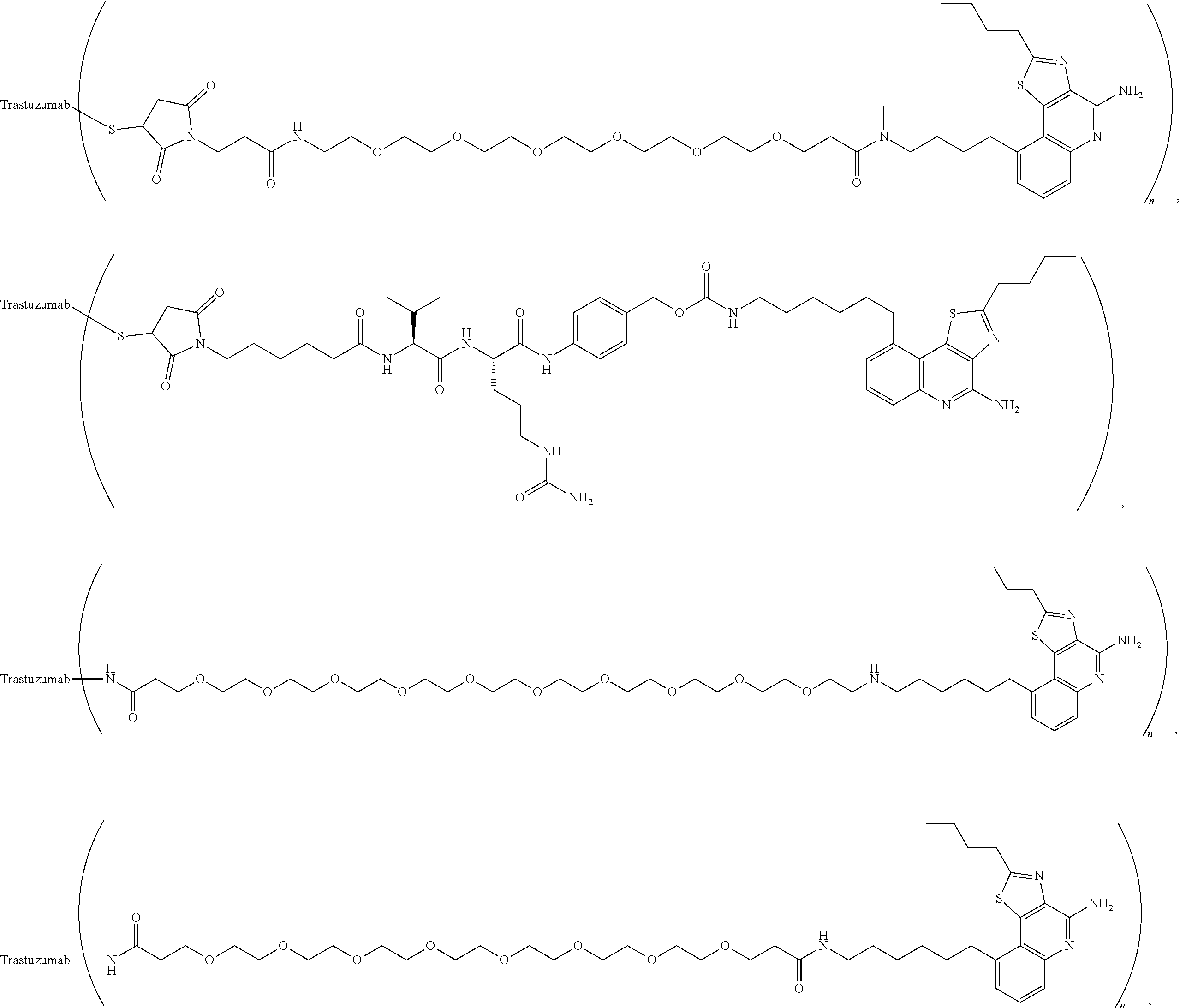 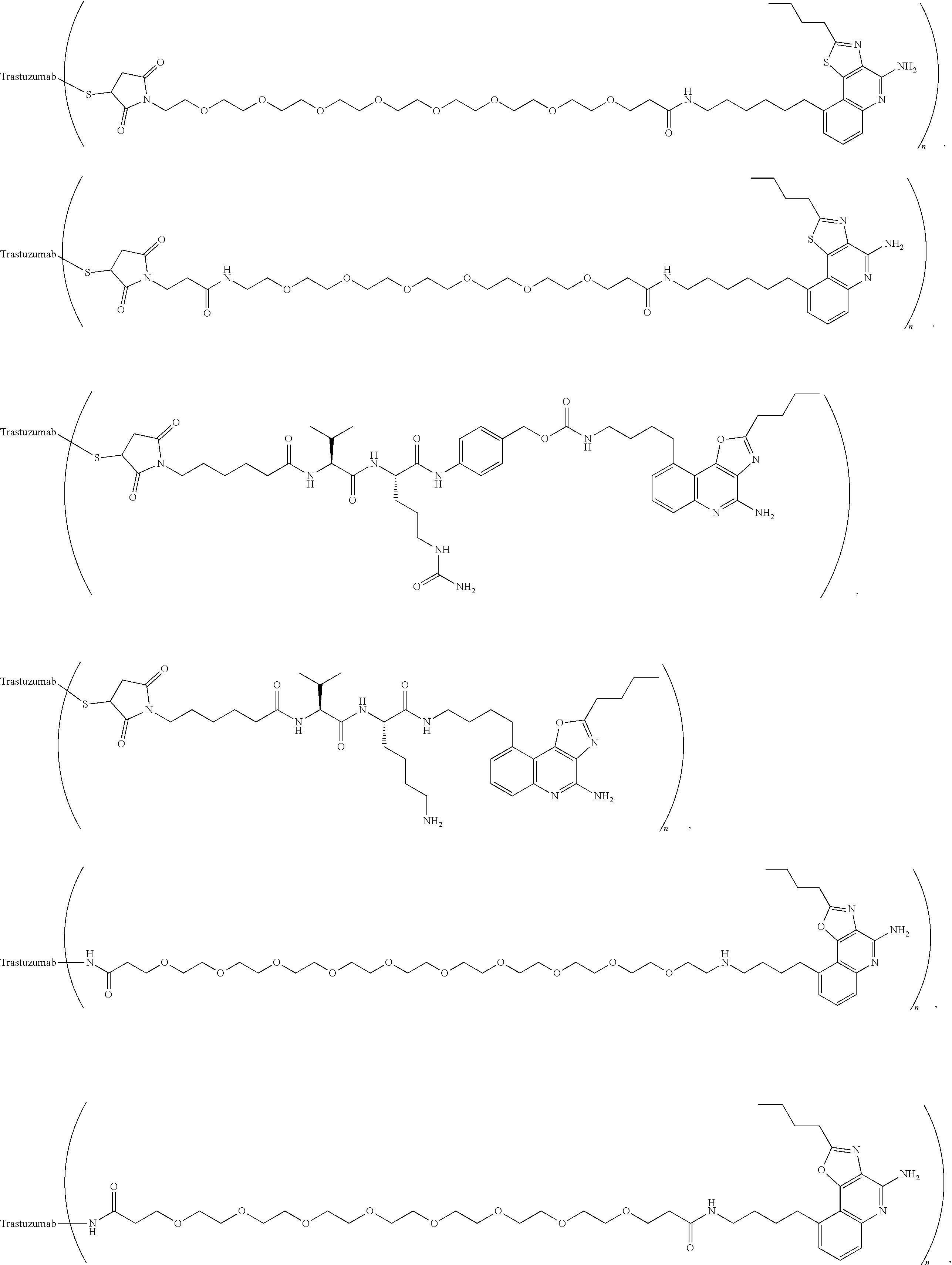 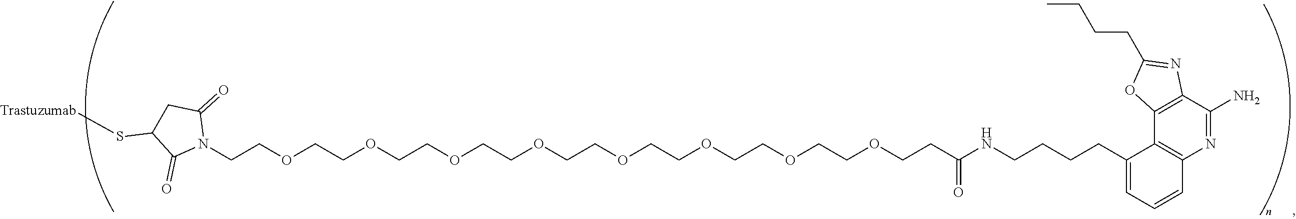 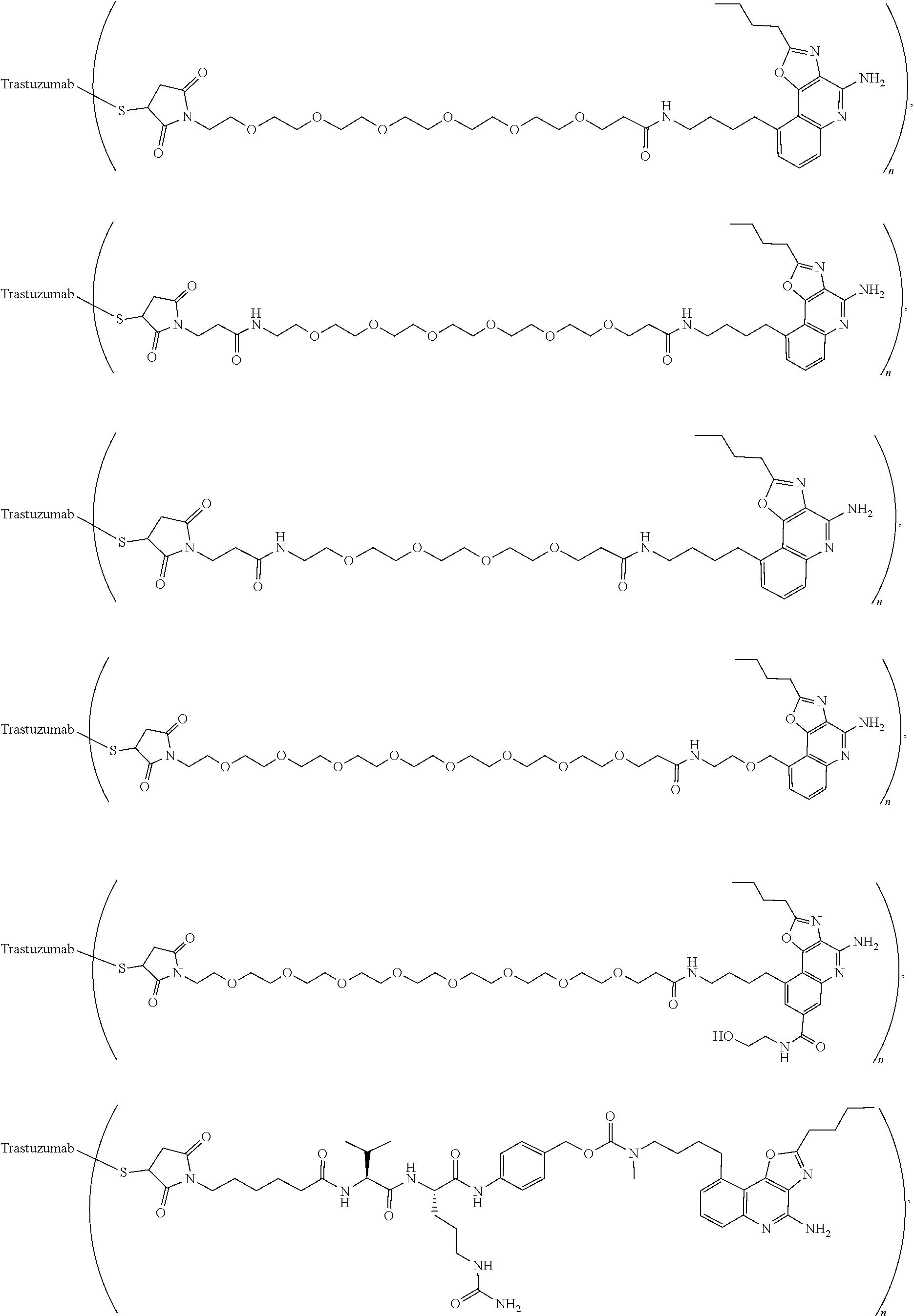 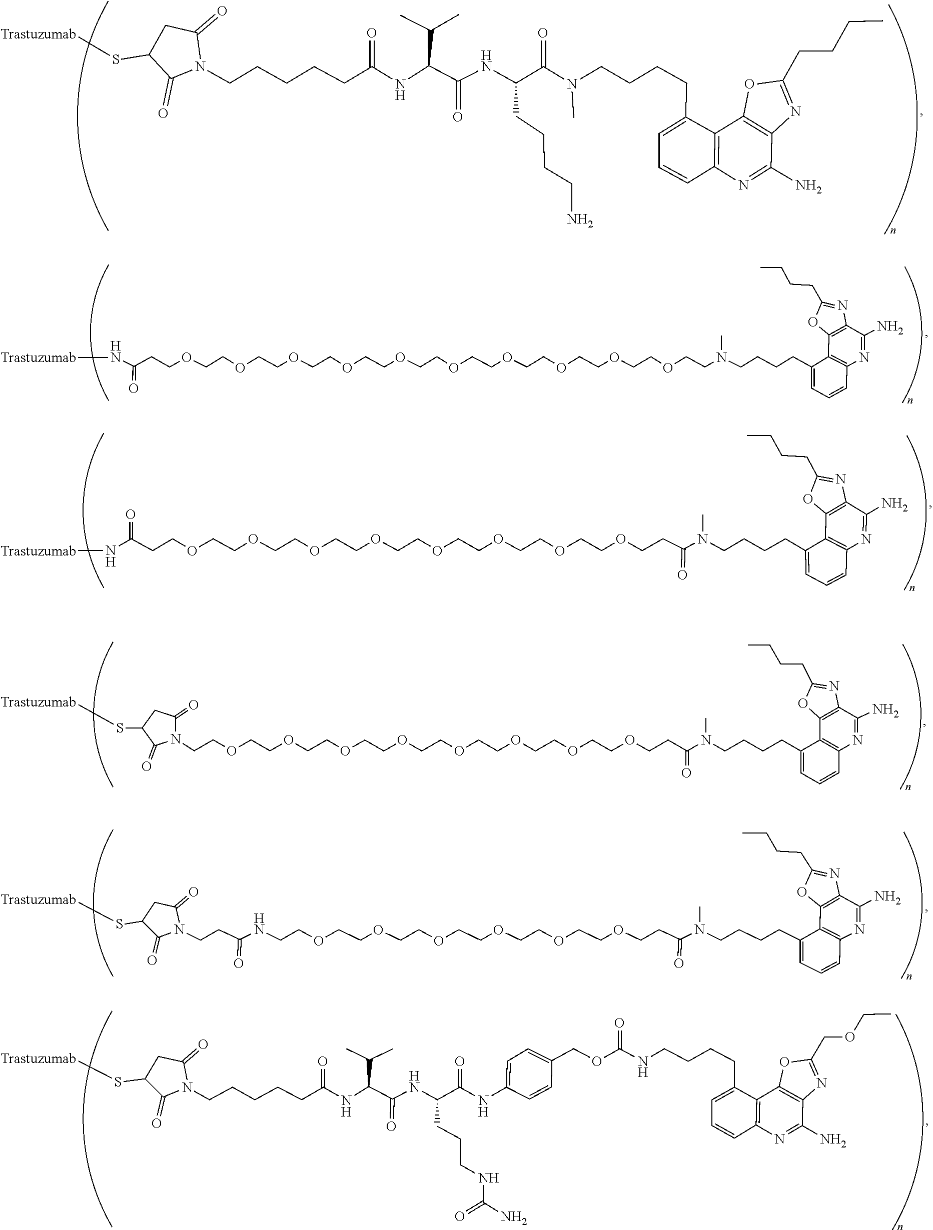 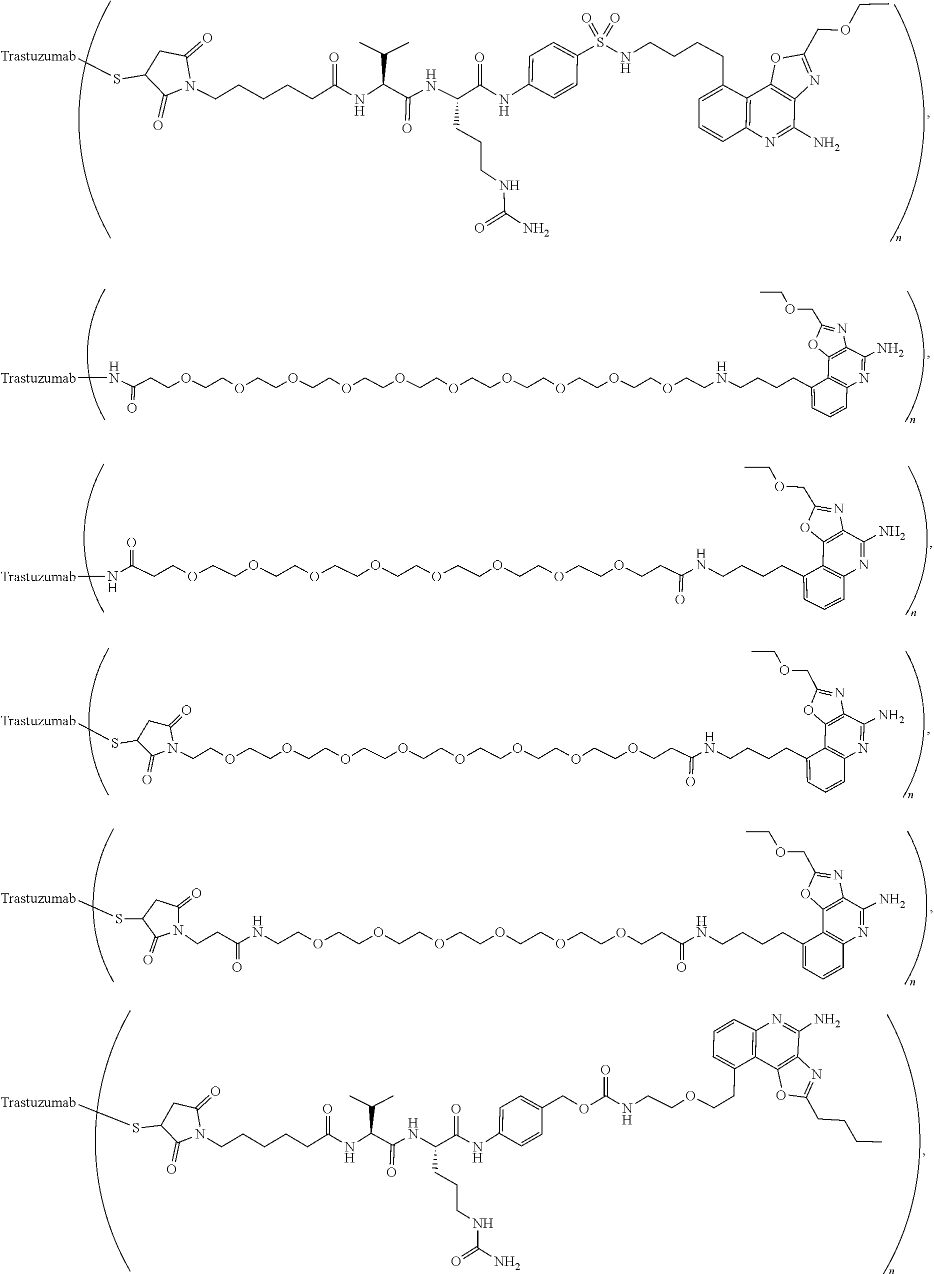 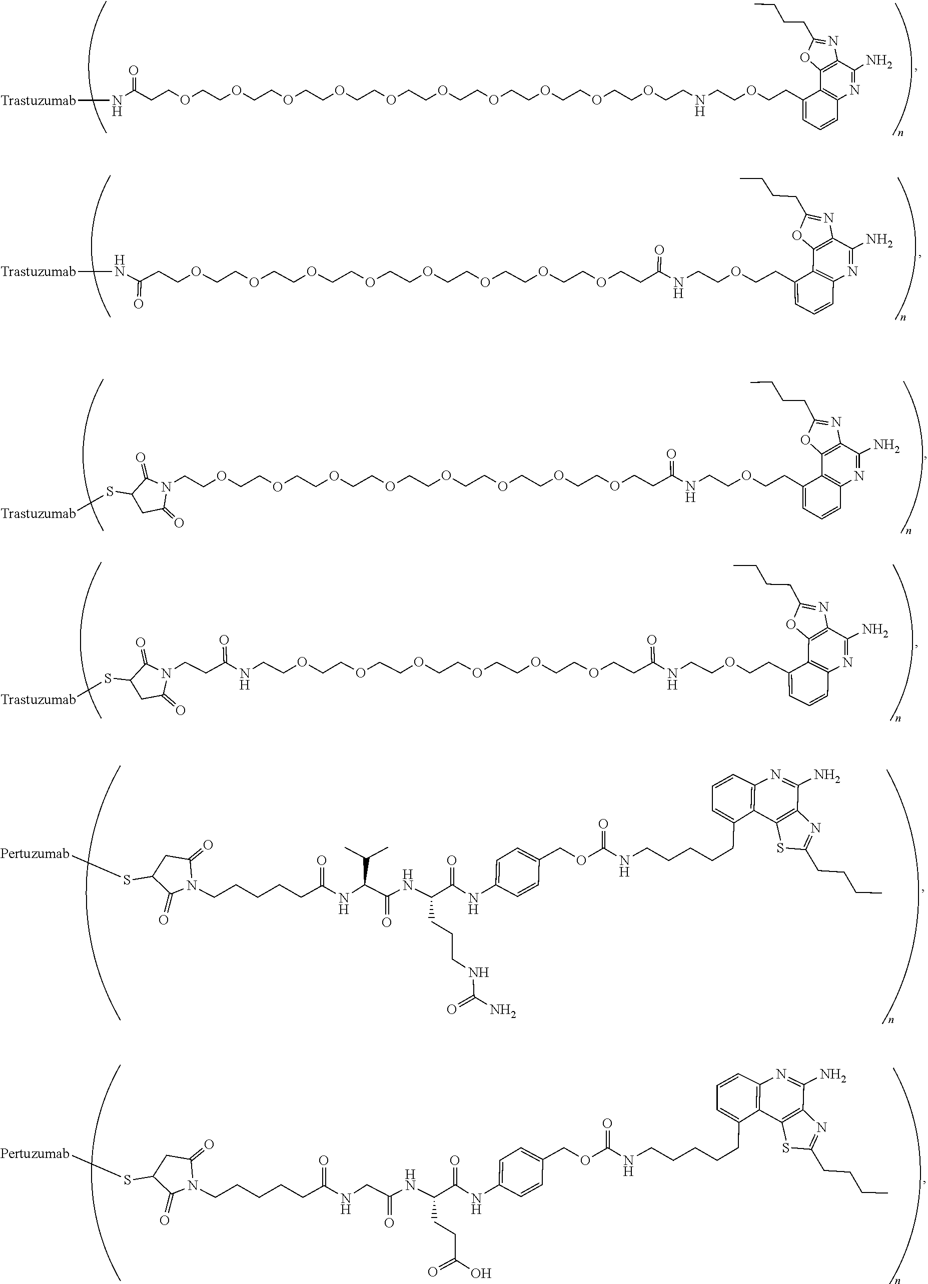 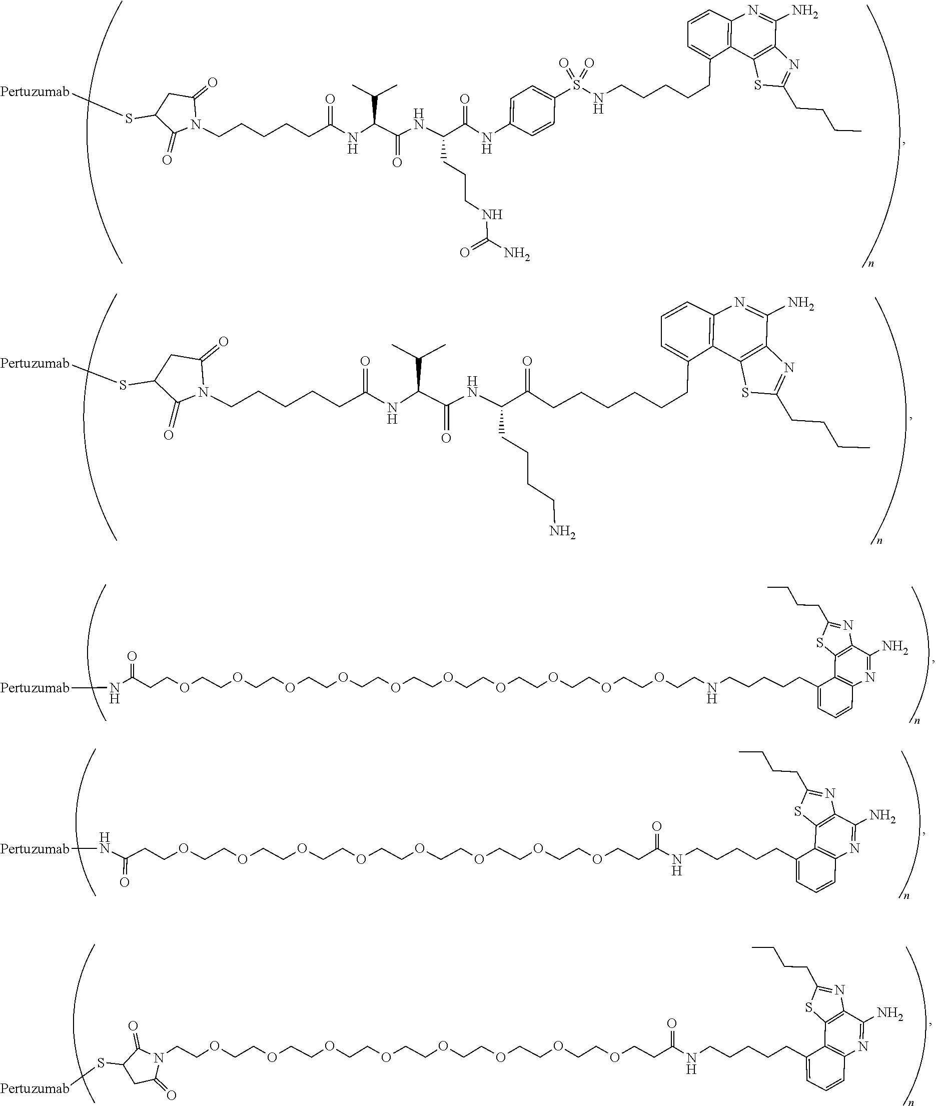 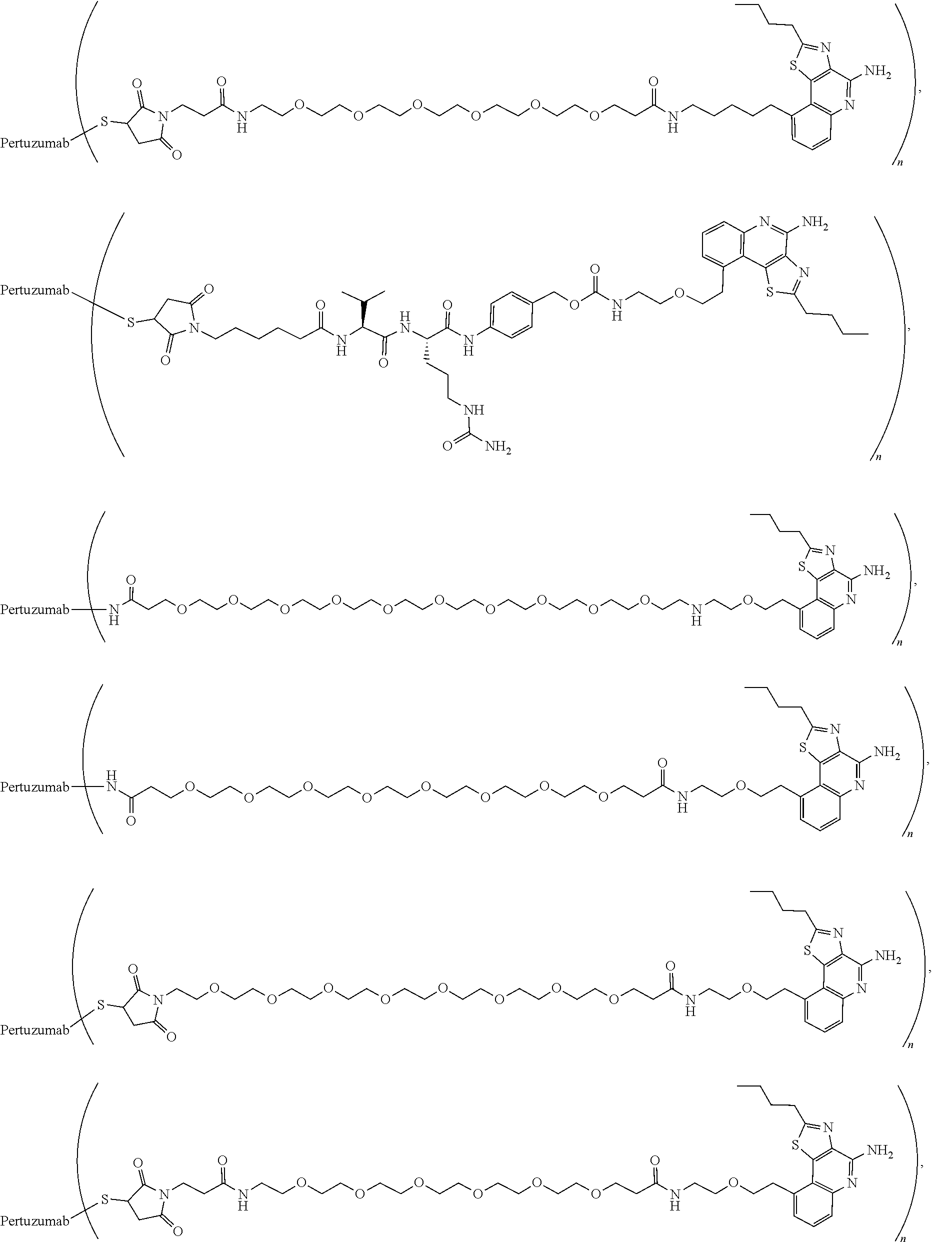 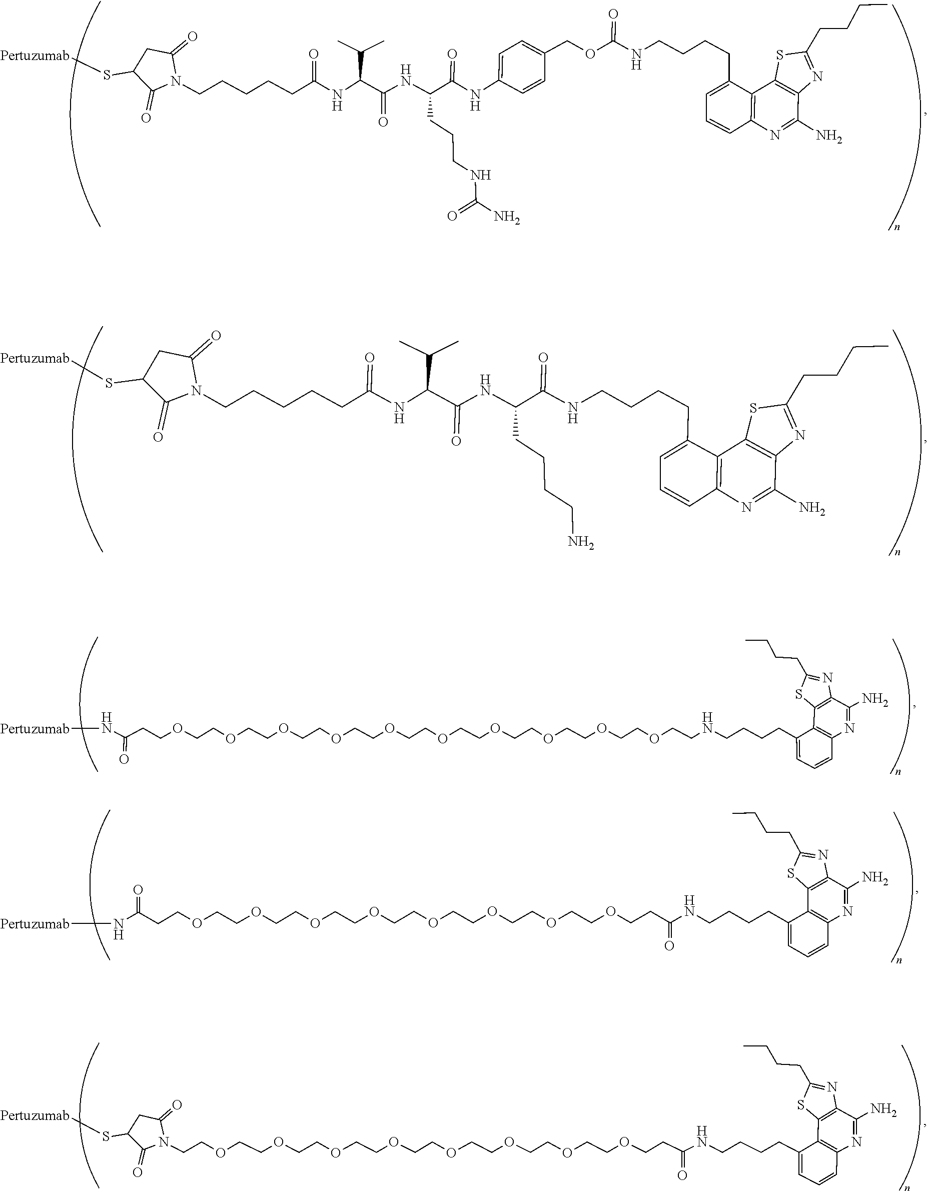 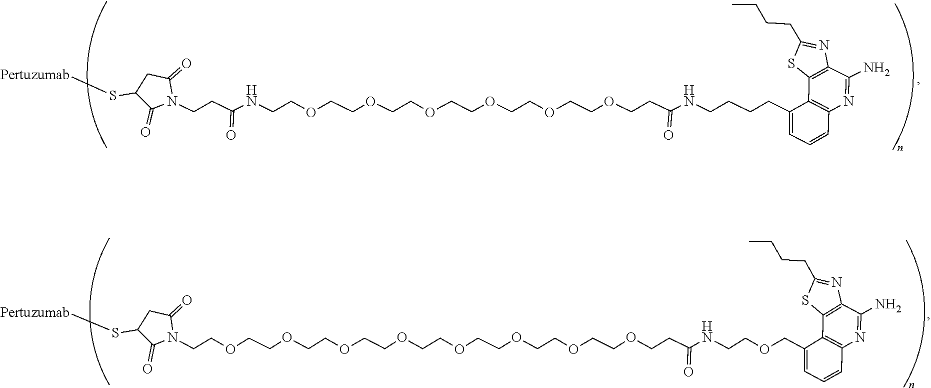 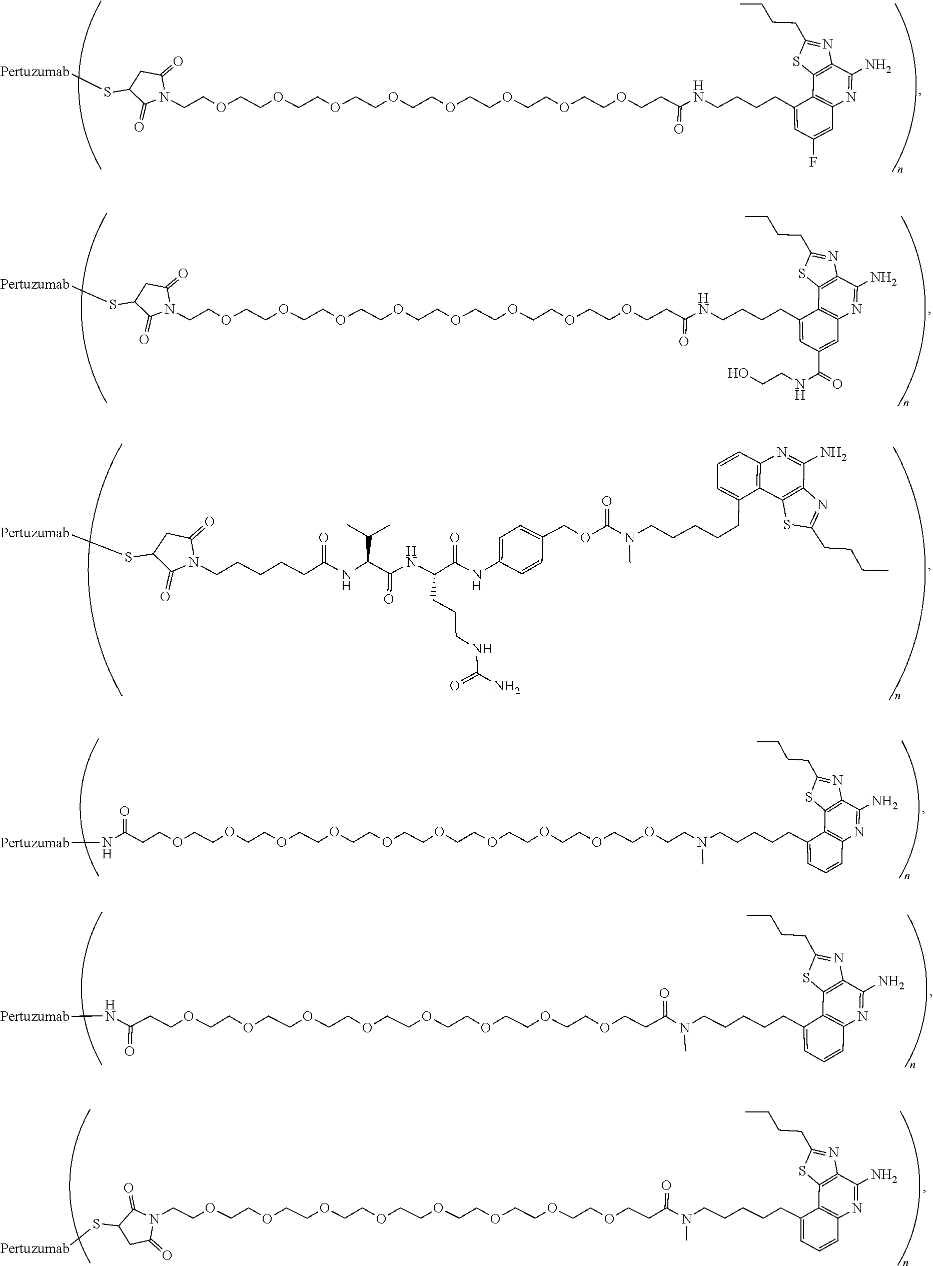 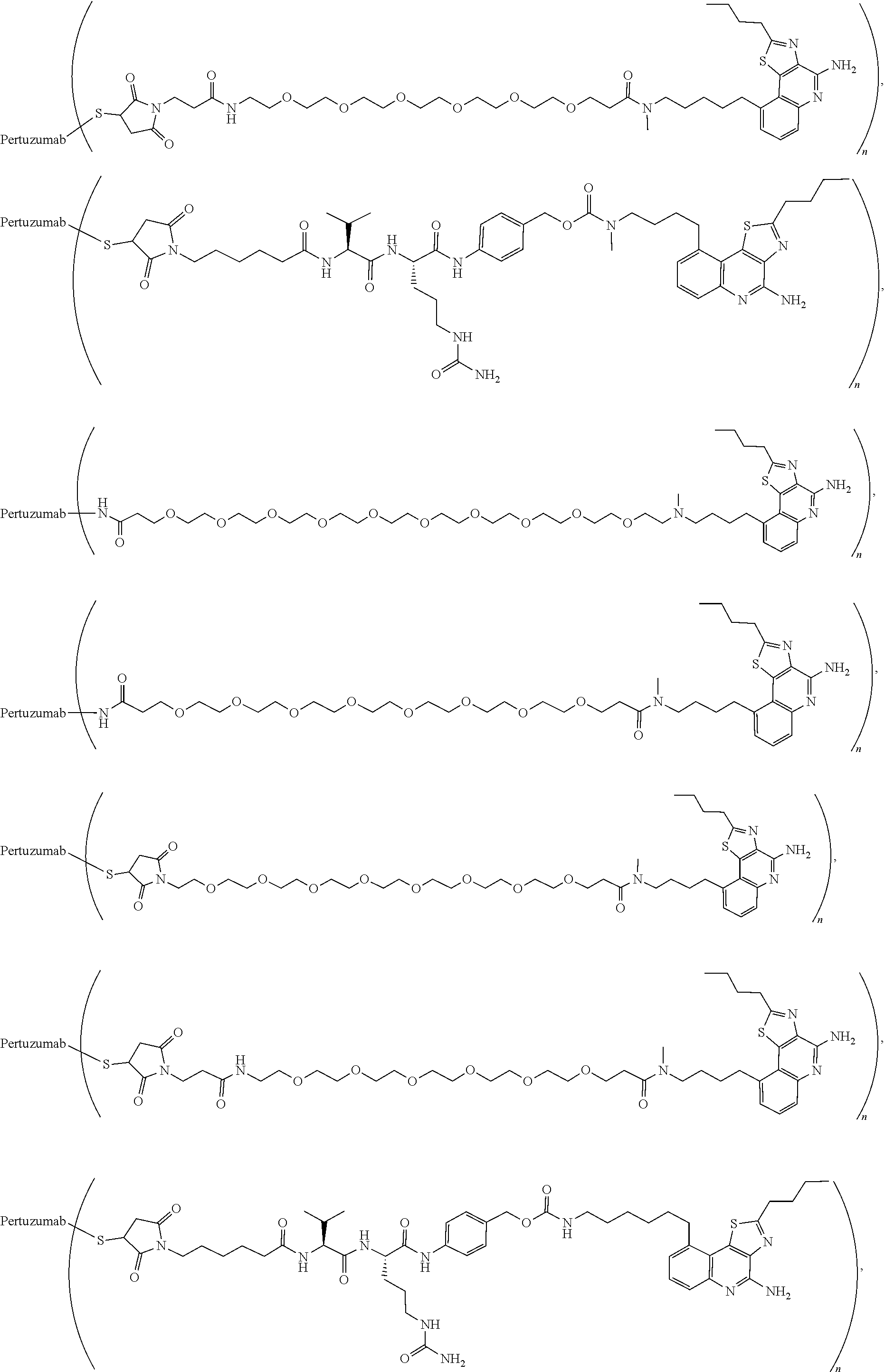 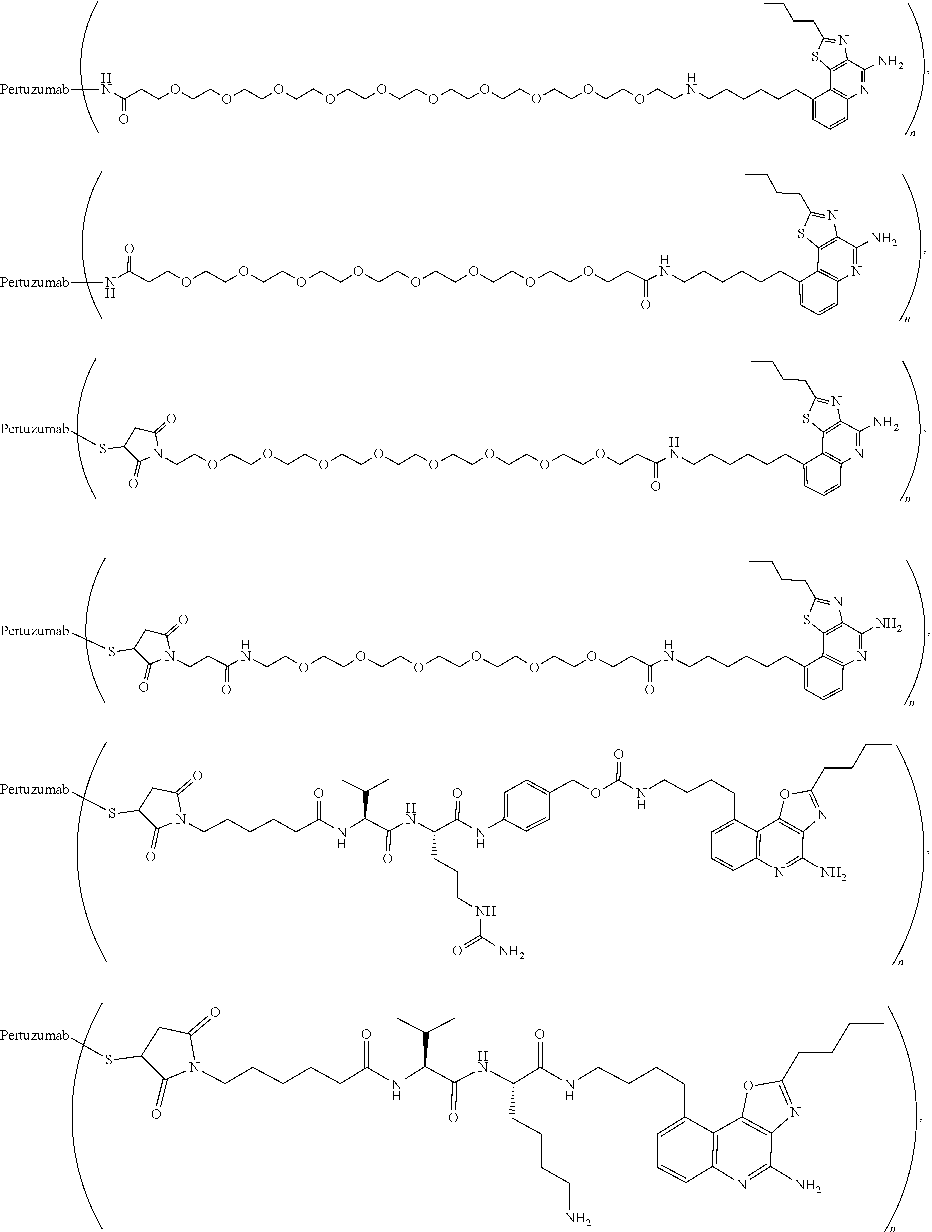 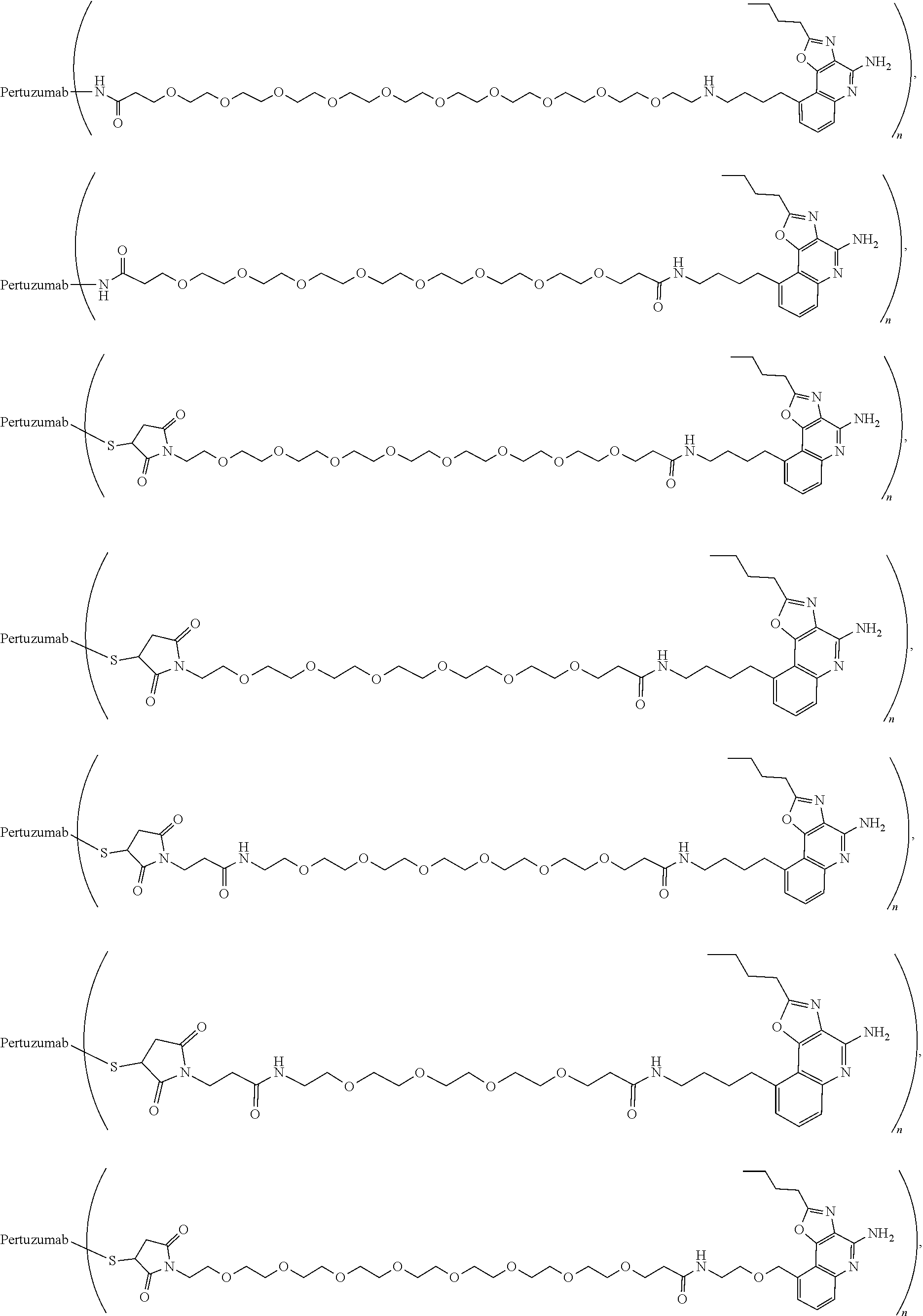 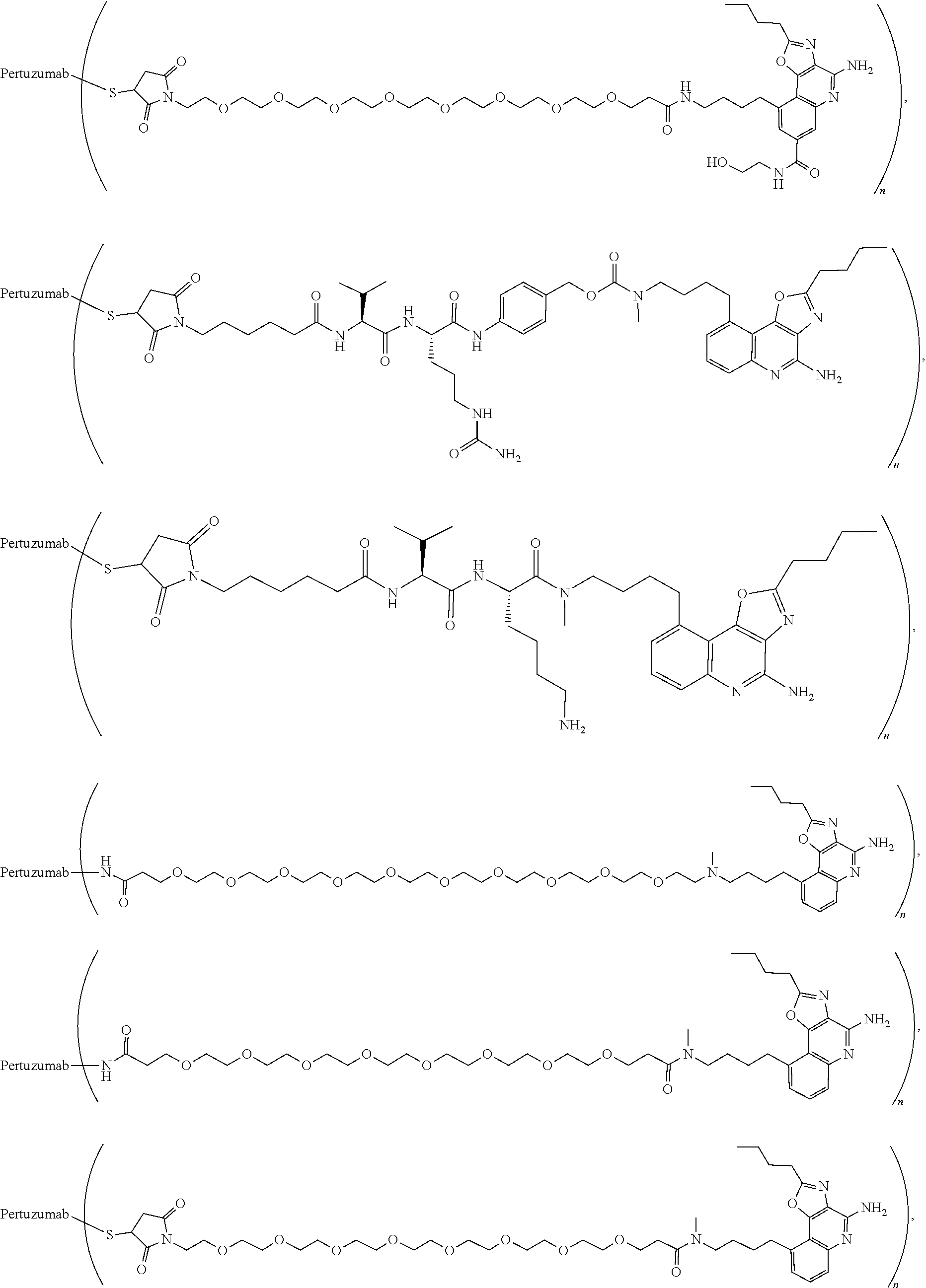 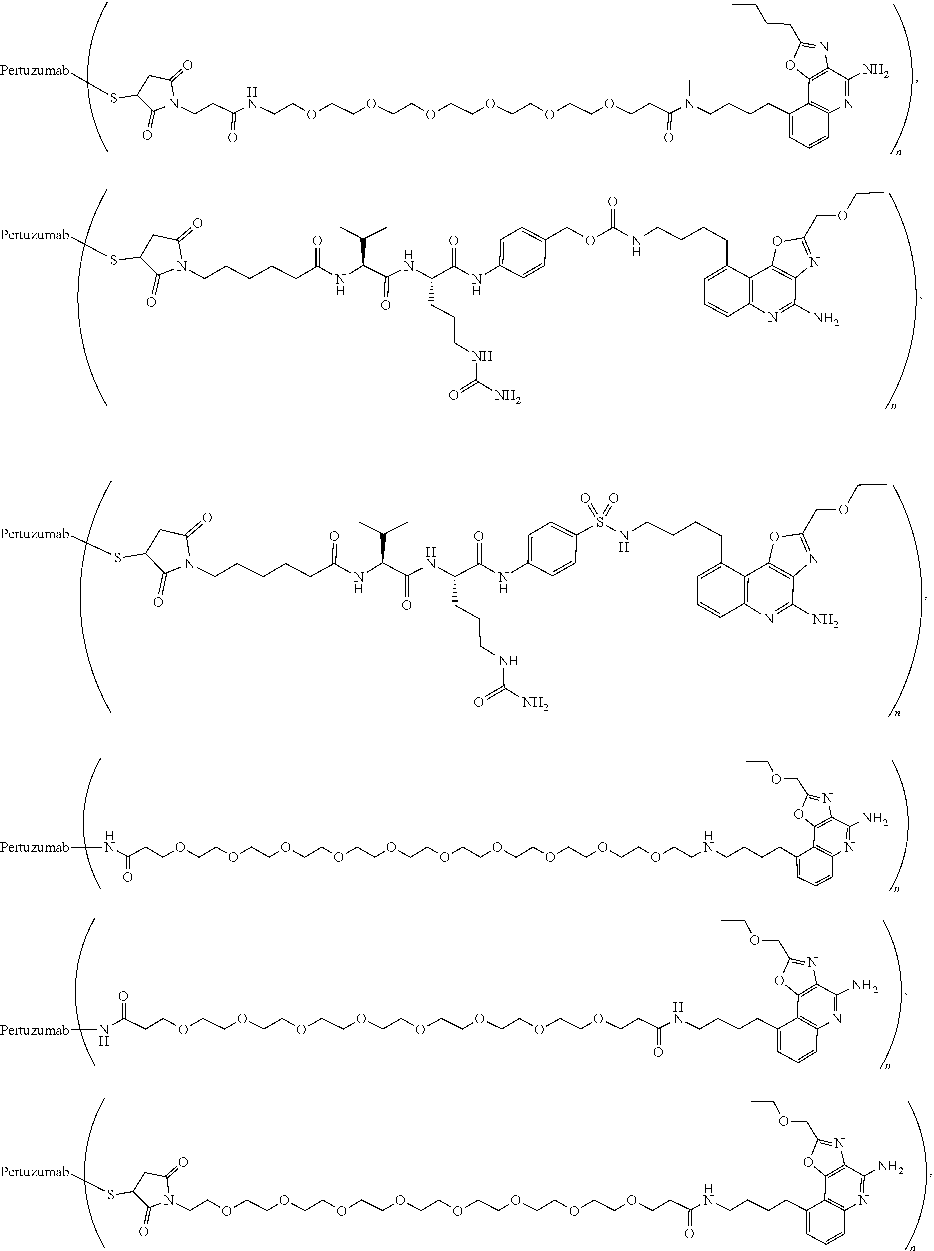 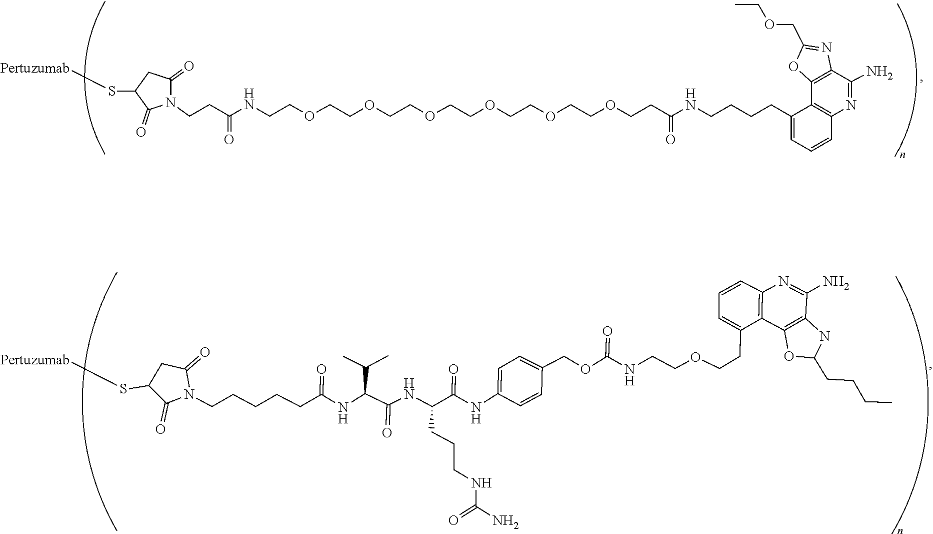 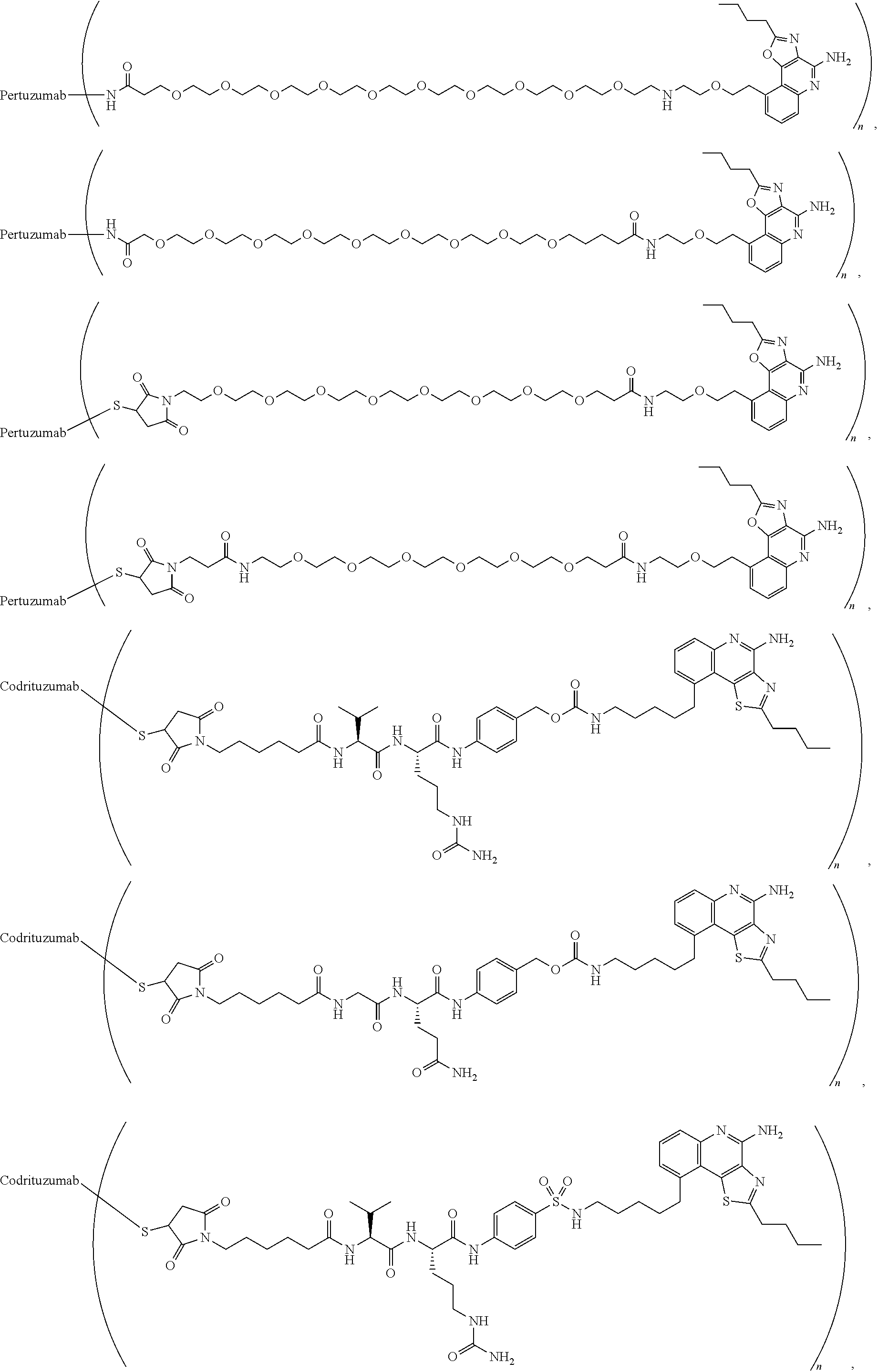 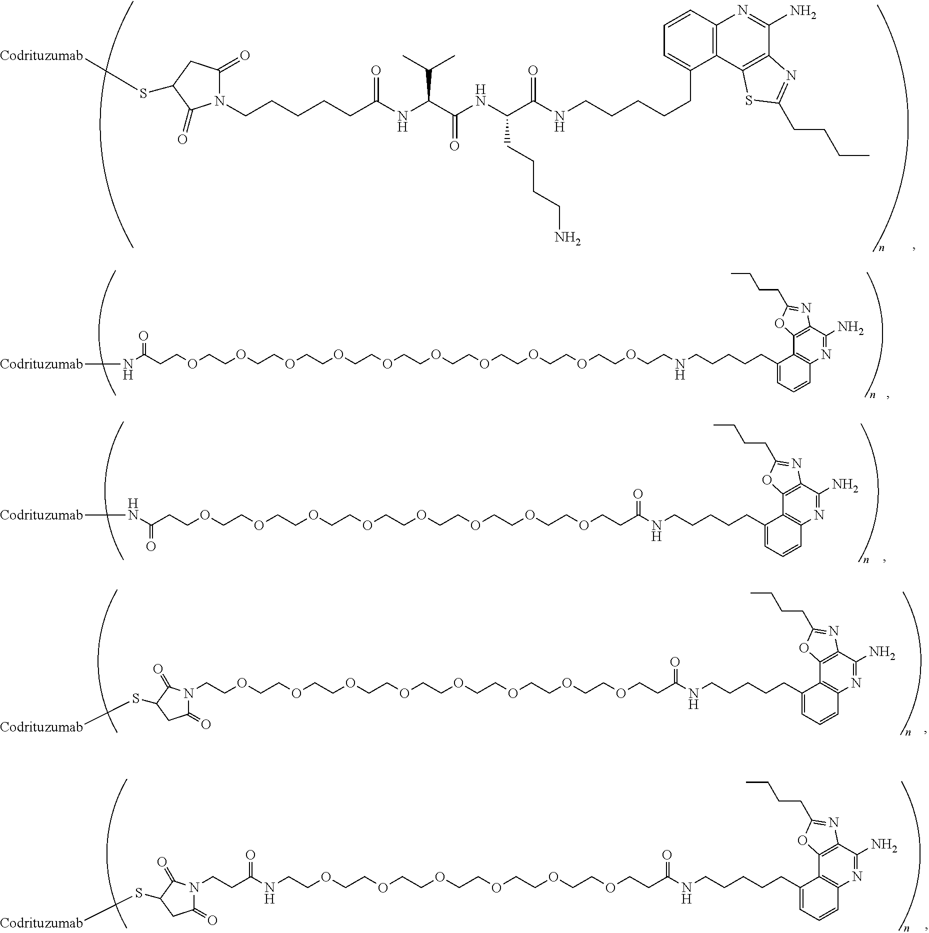 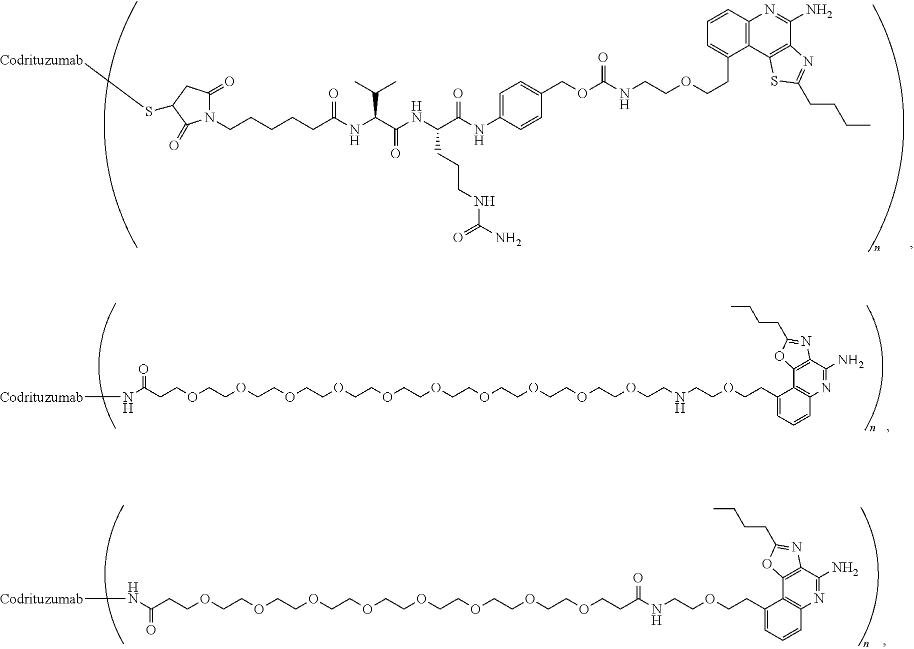 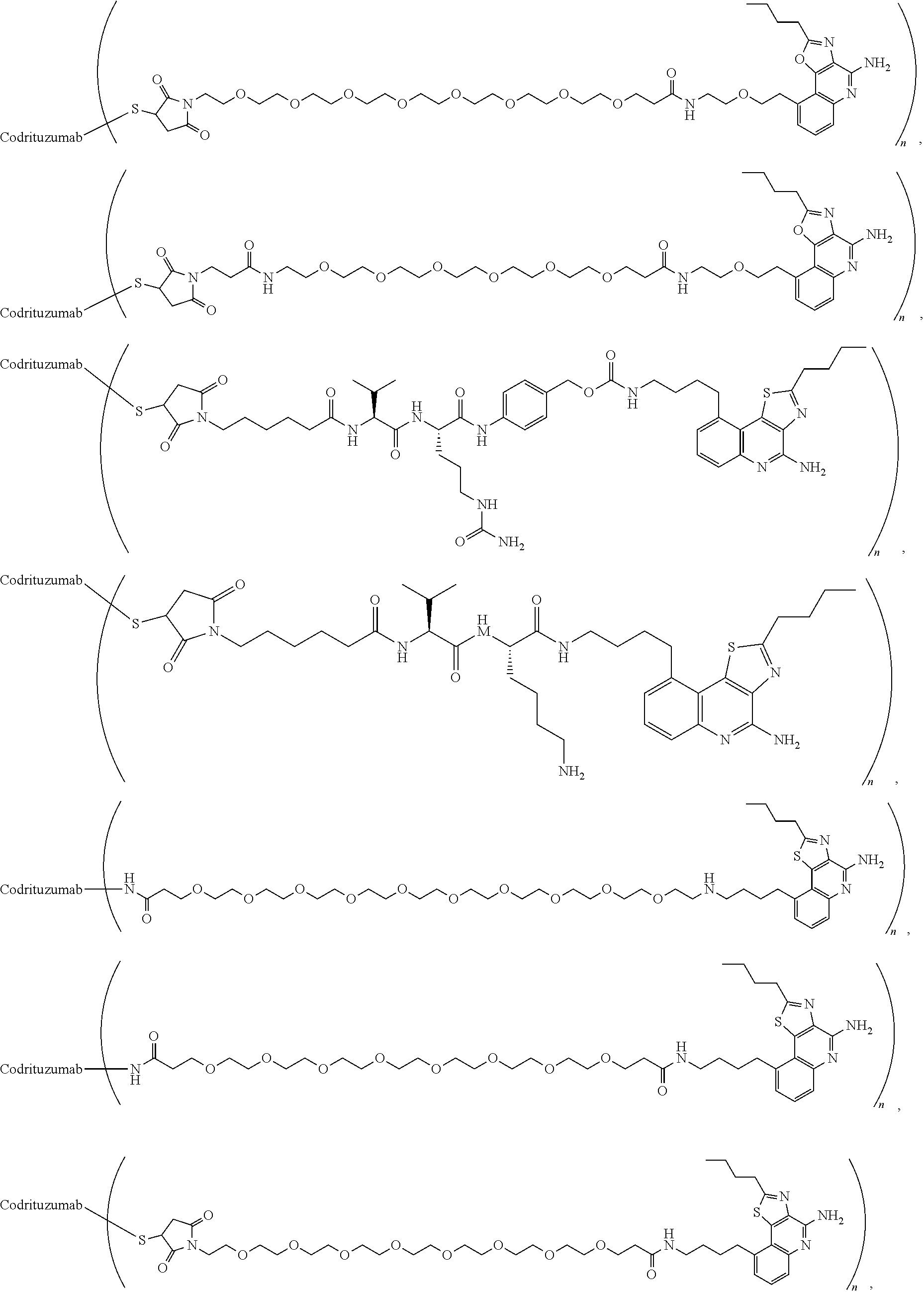 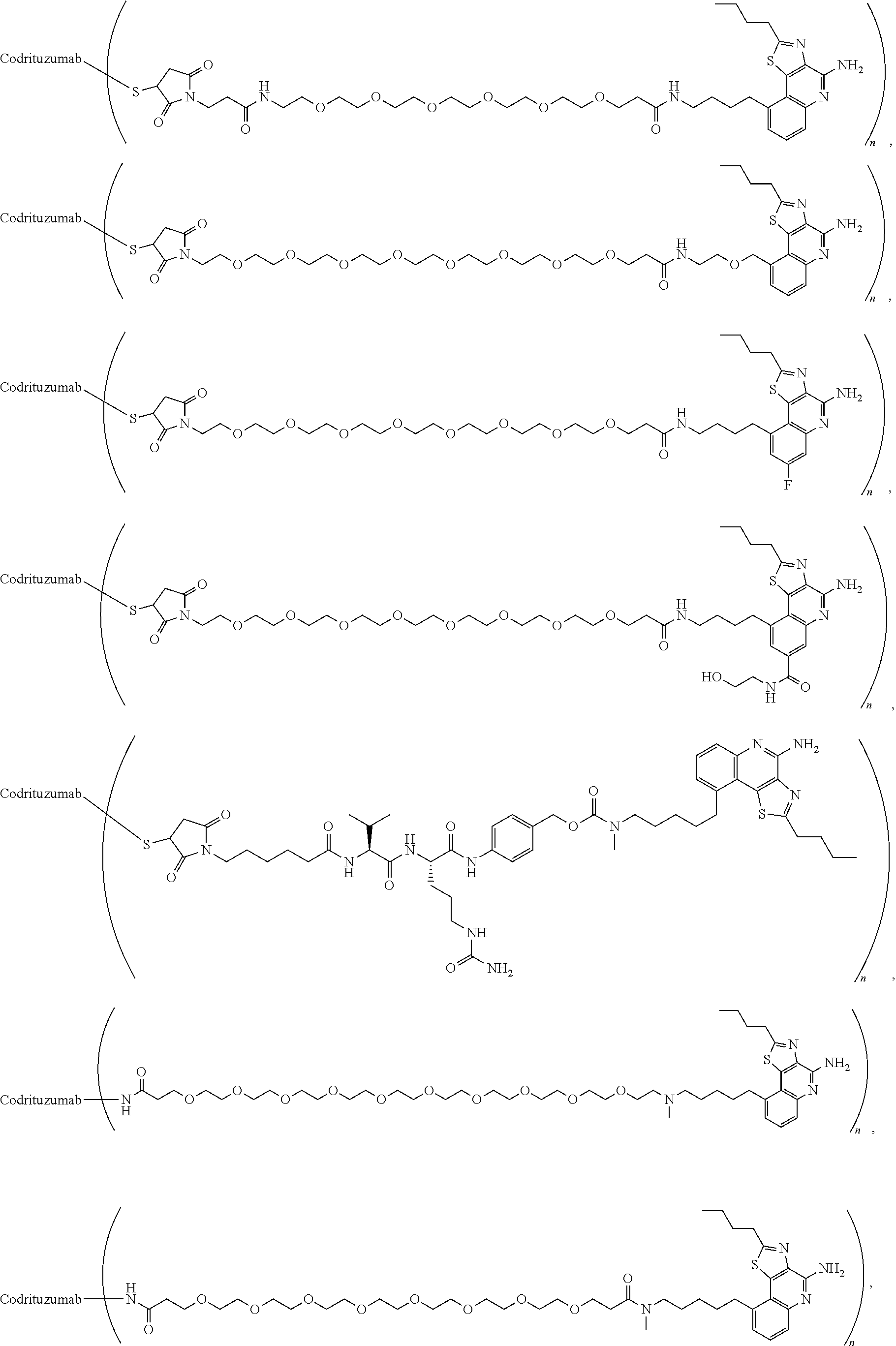 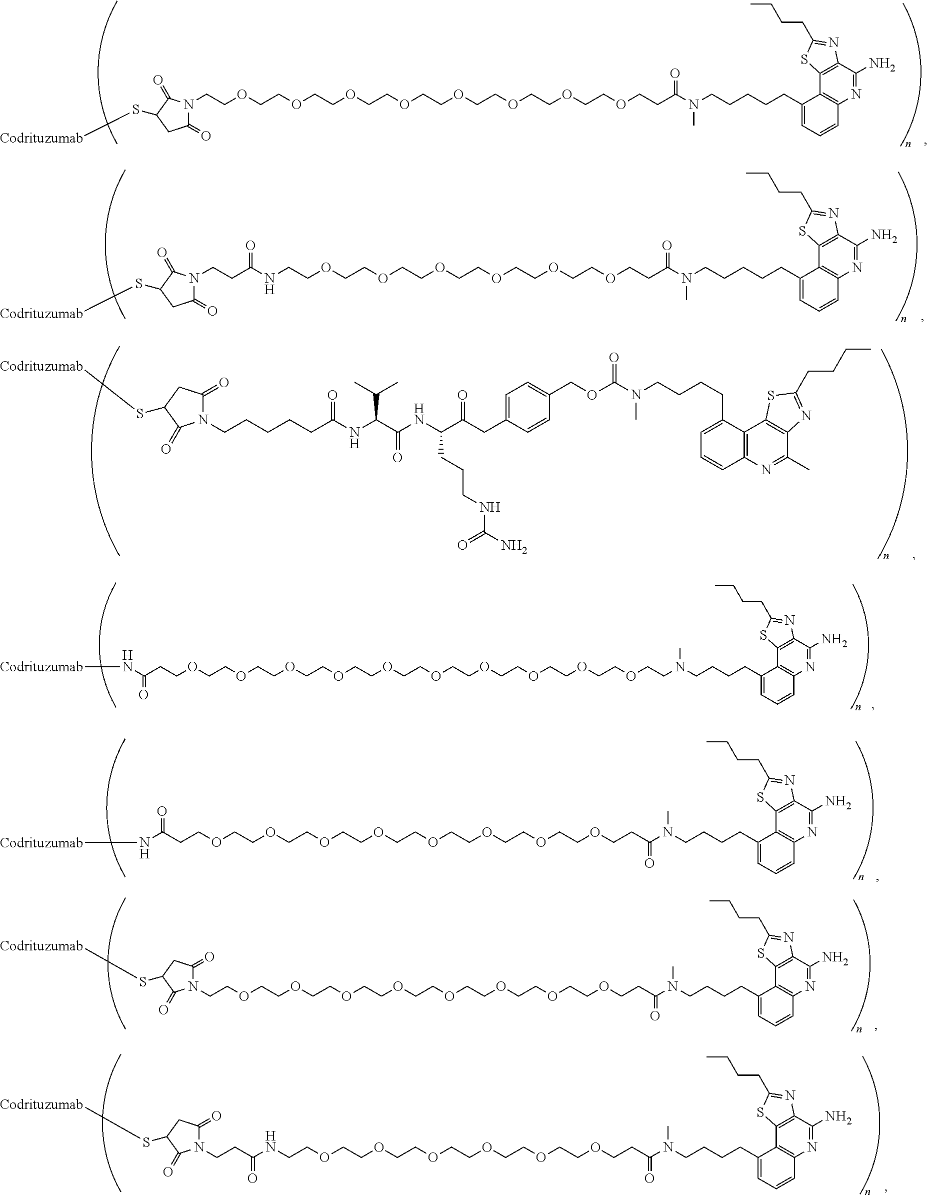 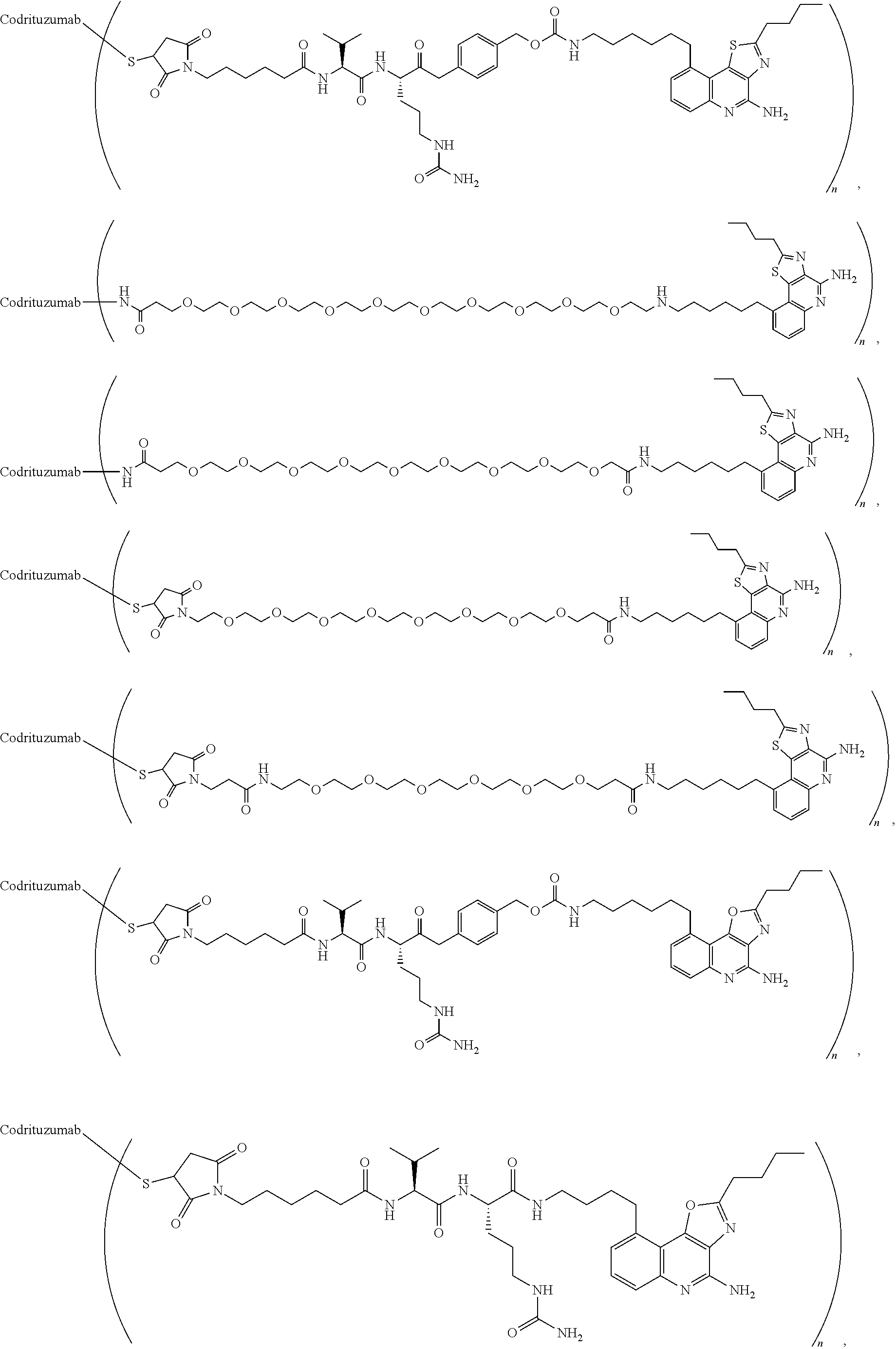 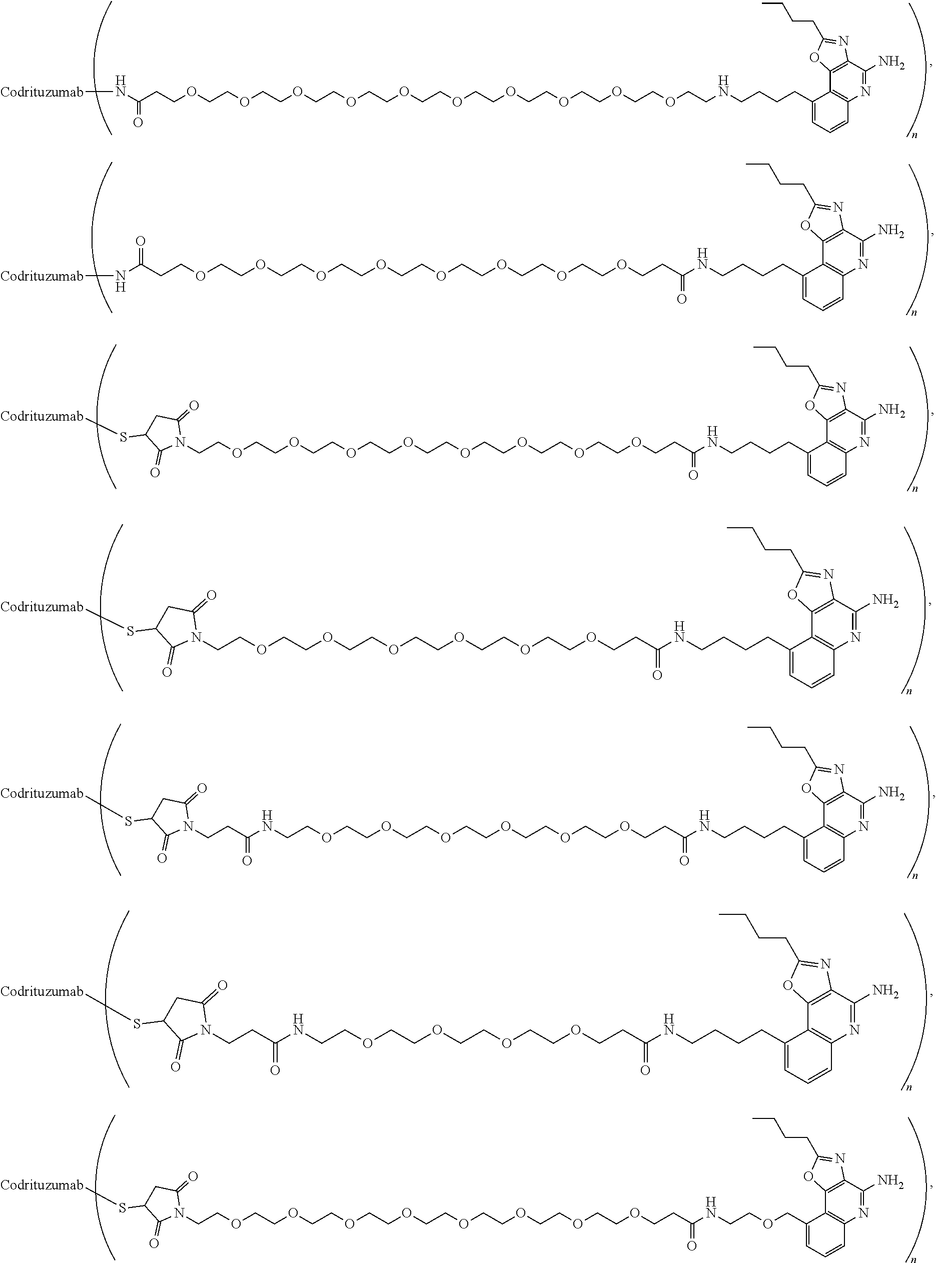 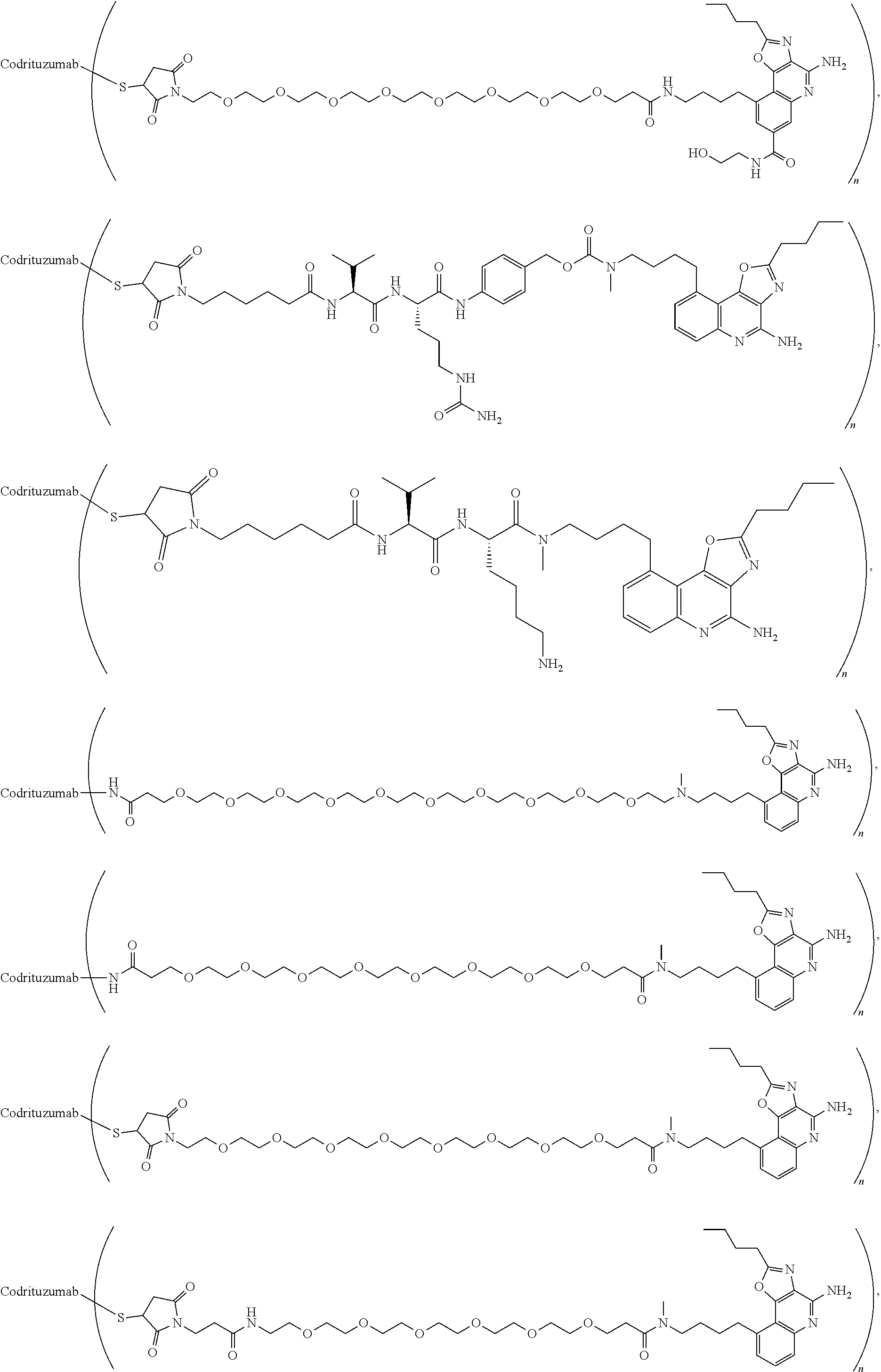 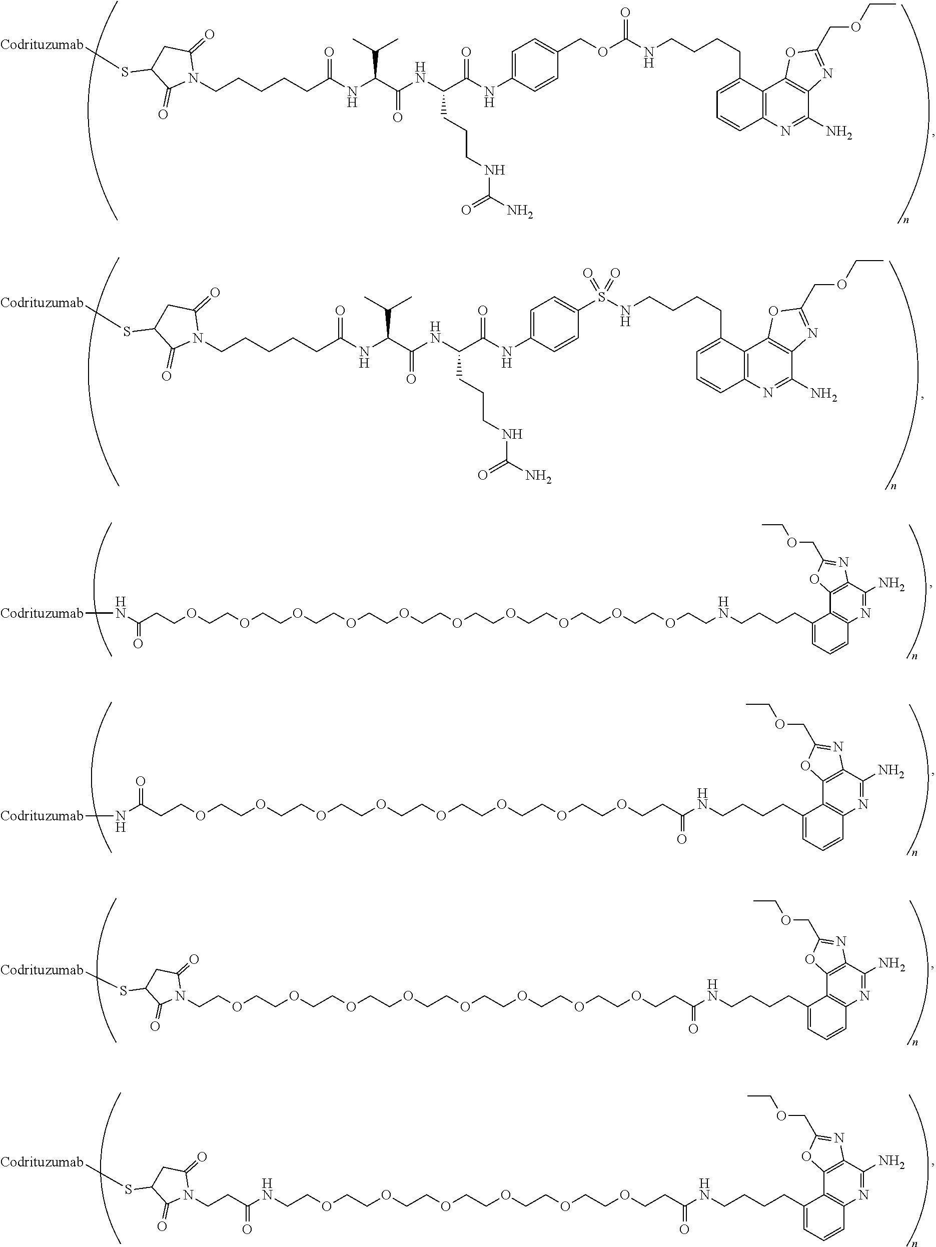 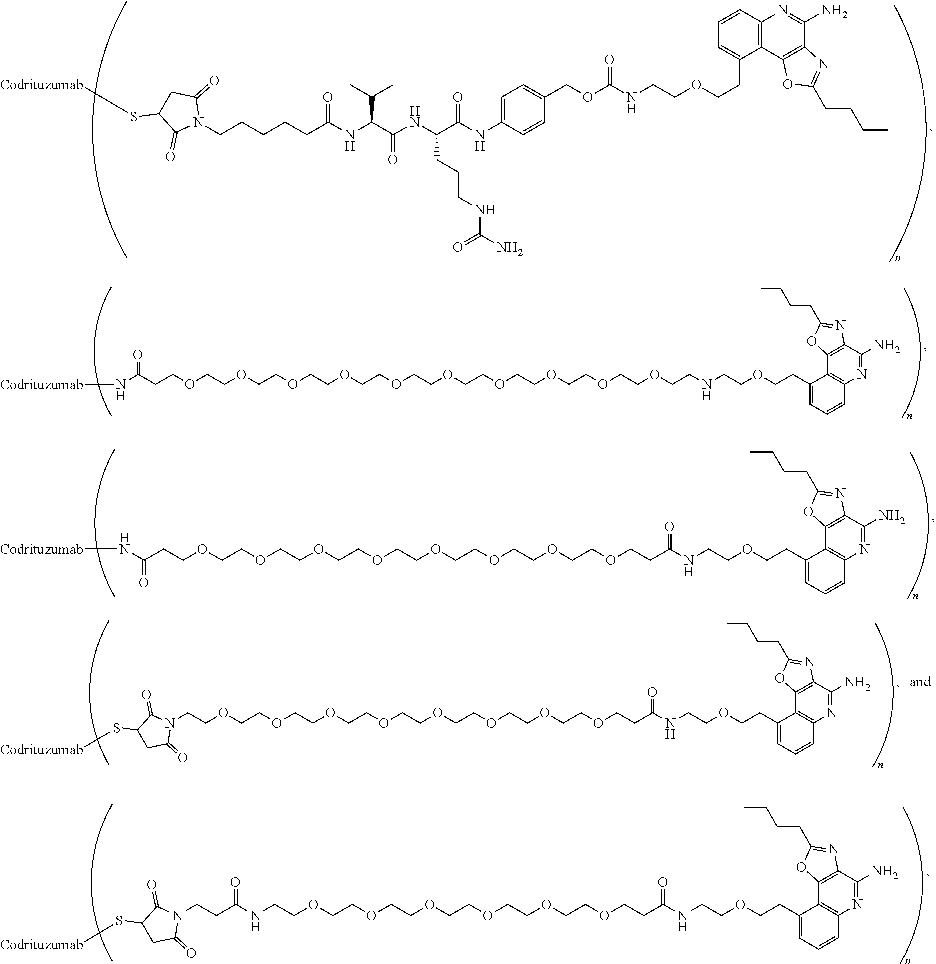
wherein
n is an integer or a decimal from 1 to 10.

66. A compound or a conjugate thereof, or an isomer thereof, or a mixture thereof, or a pharmaceutically acceptable salt thereof, wherein the compound has a structure represented by formula (III-3A): 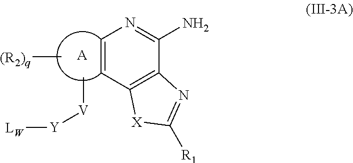
wherein
X is selected from —S— and —O—;
R₁ is selected from C₁-C₆ alkyl optionally substituted with one or more R₁₋₁, each R₁₋₁ being independently selected from: hydrogen, chlorine, bromine, iodine, hydroxy, amino, C₁-C₃ alkyl, and C₃-C₆ cycloalkyl;
the methylene units of R₁ are each independently unreplaced or replaced by a group selected from: —O—, —N(R₁₋₂)—, —C(═O)—, and —NHC(═O)—, R₁₋₂ being selected from: hydrogen or C₁-C₆ alkyl;
ring A is selected from phenyl or heteroaryl;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with one or more R₂₋₁, each R₂₋₁ being independently selected from: hydrogen, halogen, hydroxy, or amino;
V is selected from —(C(R_{V1})(R_{V2}))ₚ—, wherein R_{A1} and R_{A2} are each independently selected from hydrogen, chlorine, bromine, iodine, hydroxy, amino, C₁-C₃ alkyl, and C₃-C₆ cycloalkyl, wherein the C₁-C₃ alkyl and the C₁-C₃ cycloalkyl are each independently optionally substituted with one or more R_{V3}, each R_{V3} being independently selected from: hydrogen, halogen, hydroxy, or amino;
the methylene units of V are each independently unreplaced or replaced by a group selected from: —O—, —N(R_{V4})—, —C(═O)—, —N(R_{V4})C(═O)—, 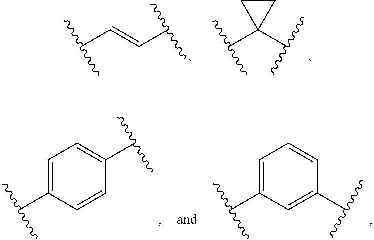
R_{V4} being selected from H, C₁-C₆ alkyl, and 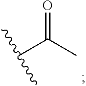 [SMILES: *C(C)=O]
Y is selected from —N(R_{y1})—, —O—, —S—, 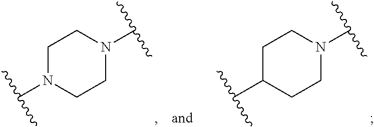 [SMILES: *N1CCN(*)CC1 ; *C1CCN(*)CC1]
R_{Y1} is selected from hydrogen and C₁-C₆ alkyl;
q is selected from 0, 1, 2, or 3;
p is selected from 1, 2, 3, 4, 5, 7, and 8;
L_{W} is -Tr-L₃-L₂-L₁;
Tr is selected from 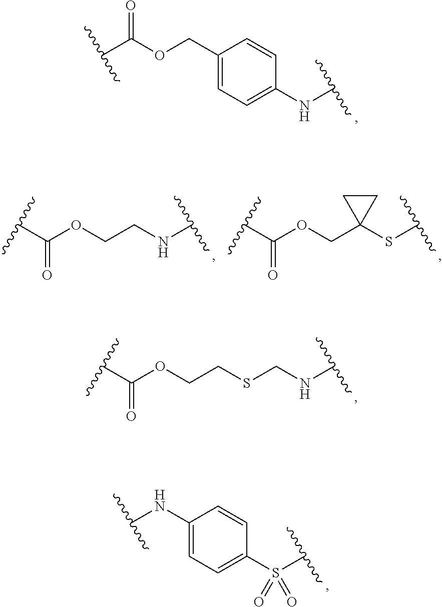 [SMILES: *Nc1ccc(COC(*)=O)cc1 ; *NCCOC(*)=O ; *SC1(COC(*)=O)CC1 ; *NCSCCOC(*)=O ; *Nc1ccc(S(*)(=O)=O)cc1]
and a single bond;
L₁ is selected from 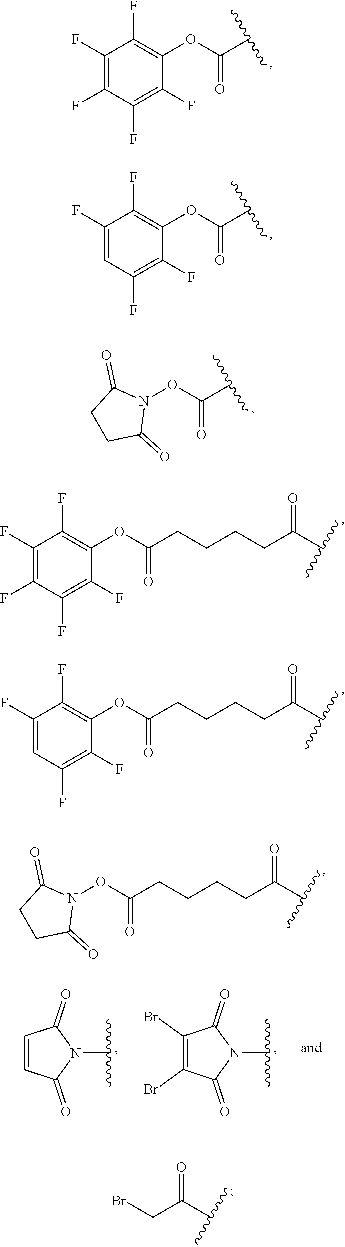
L₂ is selected from 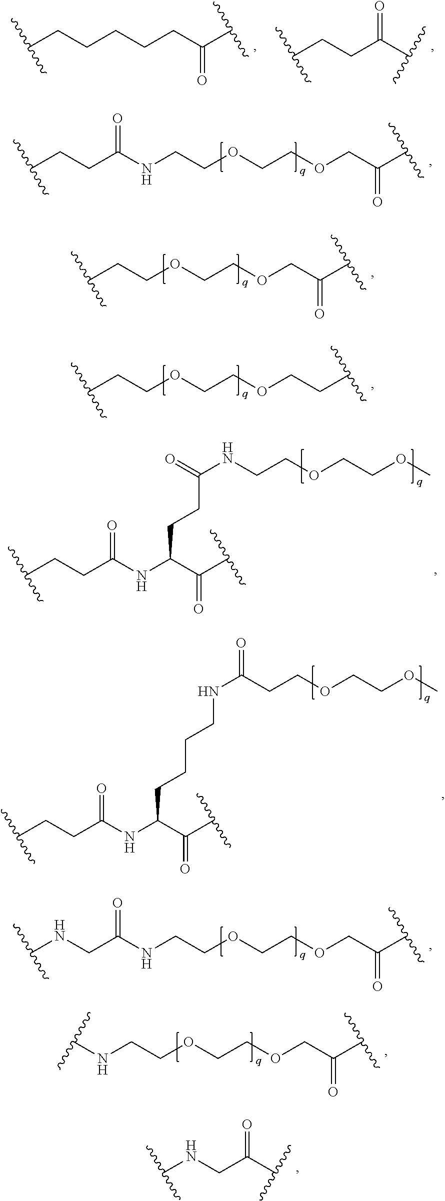
and a single bond, wherein q is an integer from 1 to 20;
L₃ is selected from a single bond or the following peptide residues: -valine-cysteine-, -valine-citrulline-, -valine-lysine-, -valine-alanine-, -alanine-alanine-, -glycine-glycine-, -glycine-glutamic acid-, glycine-glutamine, glycine-aspartic acid-, glycine-asparagine-, -glycine-glycine-glycine-, -glycine-phenylalanine-glycine-, -alanine-alanine-alanine-, -glycine-glutamic acid-glycine-, -glycine-glutamic acid-serine-, -glycine-glycine-phenylalanine-glycine-, and -glycine-glycine-glycine-glycine-.

67. The compound or the conjugate thereof, or the isomer thereof, or the mixture thereof, or the pharmaceutically acceptable salt thereof according to claim 66, wherein the compound is represented by formulas (III-3A-1) and (III-3A-2): 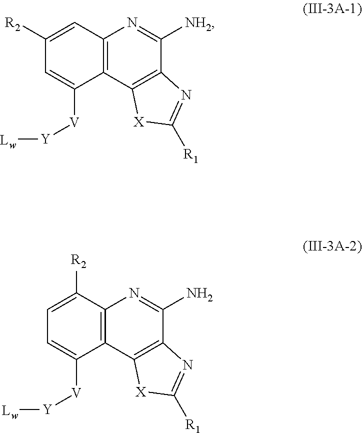
wherein
X is selected from —S— and —O—;
R₁ is selected from C₁-C₆ alkyl optionally substituted with 1, 2, or 3 RI-1, each R₁₋₁ being independently selected from: hydrogen, chlorine, bromine, iodine, hydroxy, amino, C₁-C₃ alkyl, and C₃-C₆ cycloalkyl; the methylene units of R₁ are each independently unreplaced or replaced by a group selected from: —O—, —N(CH₃)—, and —NH—;
R₂ is selected from hydrogen, chlorine, bromine, iodine, hydroxy, amino, sulfhydryl, C₁-C₃ alkyl, and C₁-C₃ alkoxy, wherein the C₁-C₃ alkyl and the C₁-C₃ alkoxy are each independently optionally substituted with 1, 2, or 3 R₂_1; each R₂-1 is independently selected from: hydrogen, halogen, hydroxy, and amino;
V is selected from C₁-C₈alkylene, preferably C₁-C₆ alkylene, wherein one or more carbon atoms in the C₁-C₈ alkylene and the C₁-C₆ alkylene may be optionally replaced by a heteroatom selected from N, O, or S, and the C₁-C₈ alkylene and the C₁-C₆ alkylene are optionally substituted with one or more substituents selected from: —NH(C₁-C₆ alkyl), —N(C₁-C₆ alkyl)₂, C₁-C₆ alkyl, oxo, hydroxy, C₁-C₆ alkoxy, —C₁-C₆ alkylhydroxy, —C₁-C₆ alkyl-C₁-C₆ alkoxy, halogen, halogenated C₁-C₆ alkyl, or halogenated C₁-C₆ alkoxy;
Y is —N(R_{Y1})—;
R_{Y1} is selected from hydrogen and C₁-C₆ alkyl; L_{W} is selected from

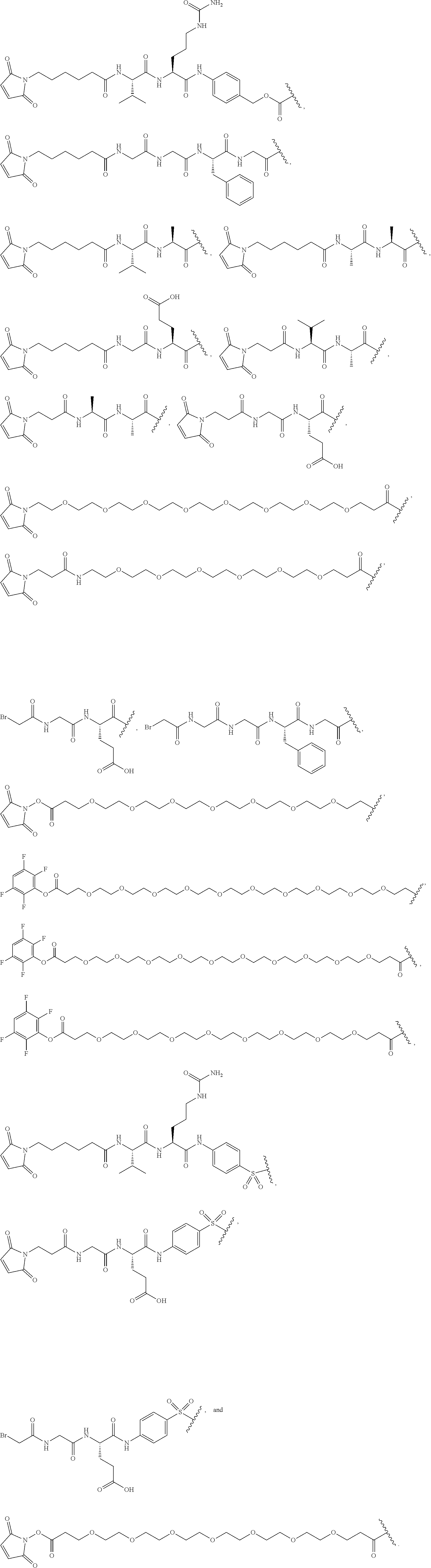

68. A compound or a conjugate thereof, or an isomer thereof, or a mixture thereof, or a pharmaceutically acceptable salt thereof, selected from:

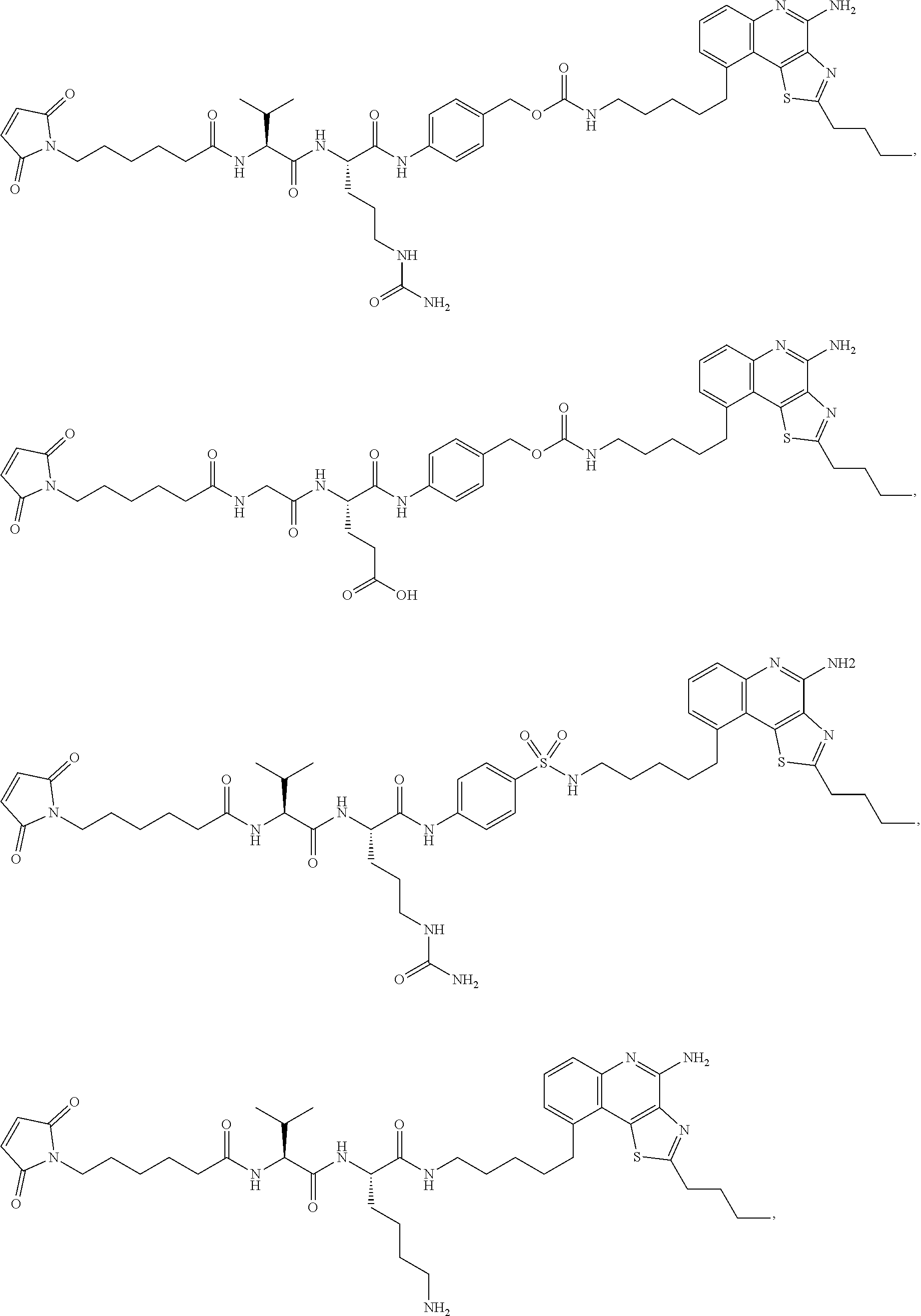 [SMILES: CCCCc1nc2c(N)nc3cccc(CCCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCNC(=O)OCc4ccc(NC(=O)[C@H](CCC(=O)O)NC(=O)CNC(=O)CCCCCN5C(=O)C=CC5=O)cc4)c3c2s1 ; CCCCc1nc2c(N)nc3cccc(CCCCCNS(=O)(=O)c4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCNC(=O)[C@H](CCCCN)NC(=O)[C@@H](NC(=O)CCCCCN4C(=O)C=CC4=O)C(C)C)c3c2s1]

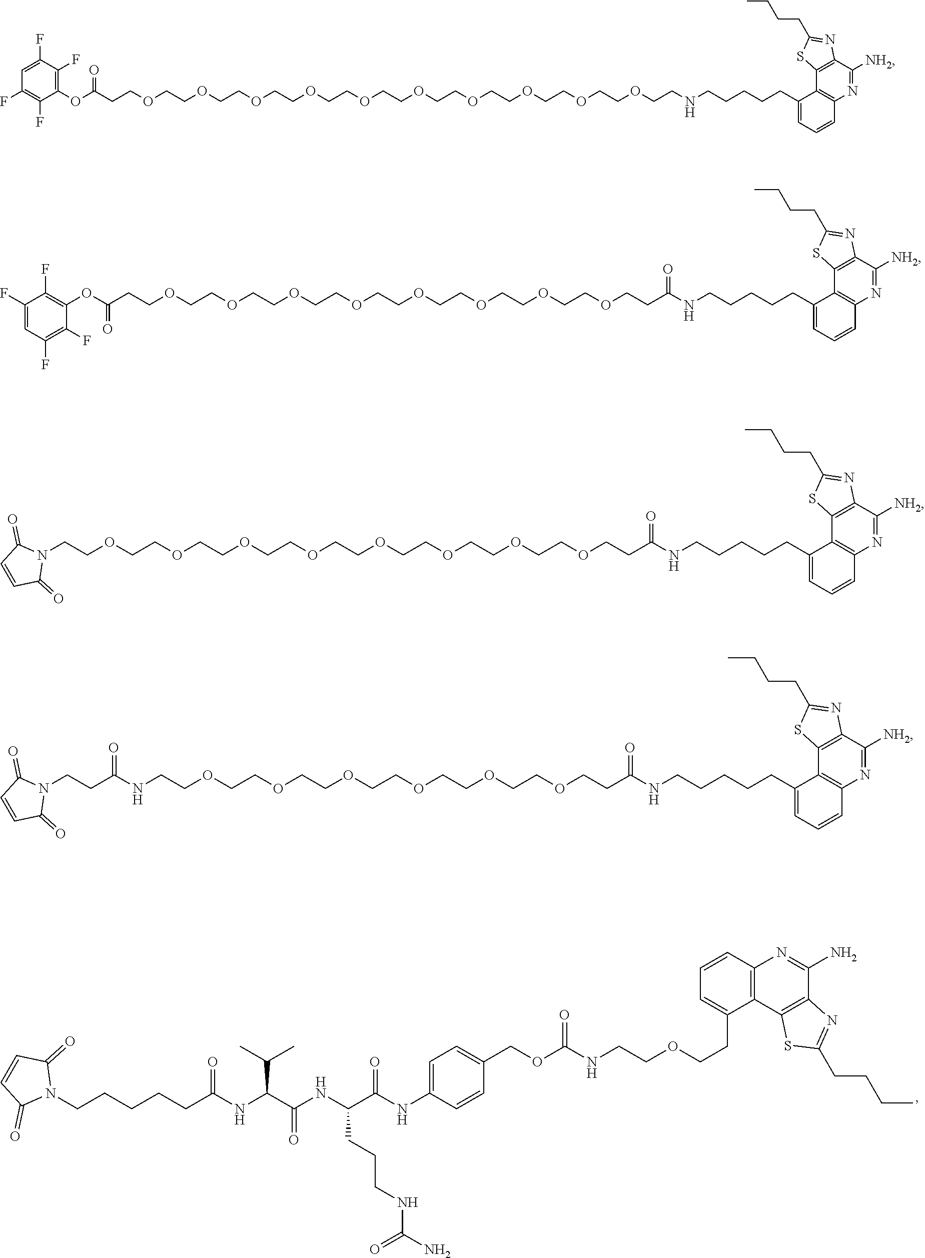 [SMILES: CCCCc1nc2c(N)nc3cccc(CCCCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCNC(=O)CCOCCOCCOCCOCCOCCOCCOCCOCCN4C(=O)C=CC4=O)c3c2s1 ; CCCCc1nc2c(N)nc3cccc(CCCCCNC(=O)CCOCCOCCOCCOCCOCCOCCNC(=O)CCN4C(=O)C=CC4=O)c3c2s1 ; CCCCc1nc2c(N)nc3cccc(CCOCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

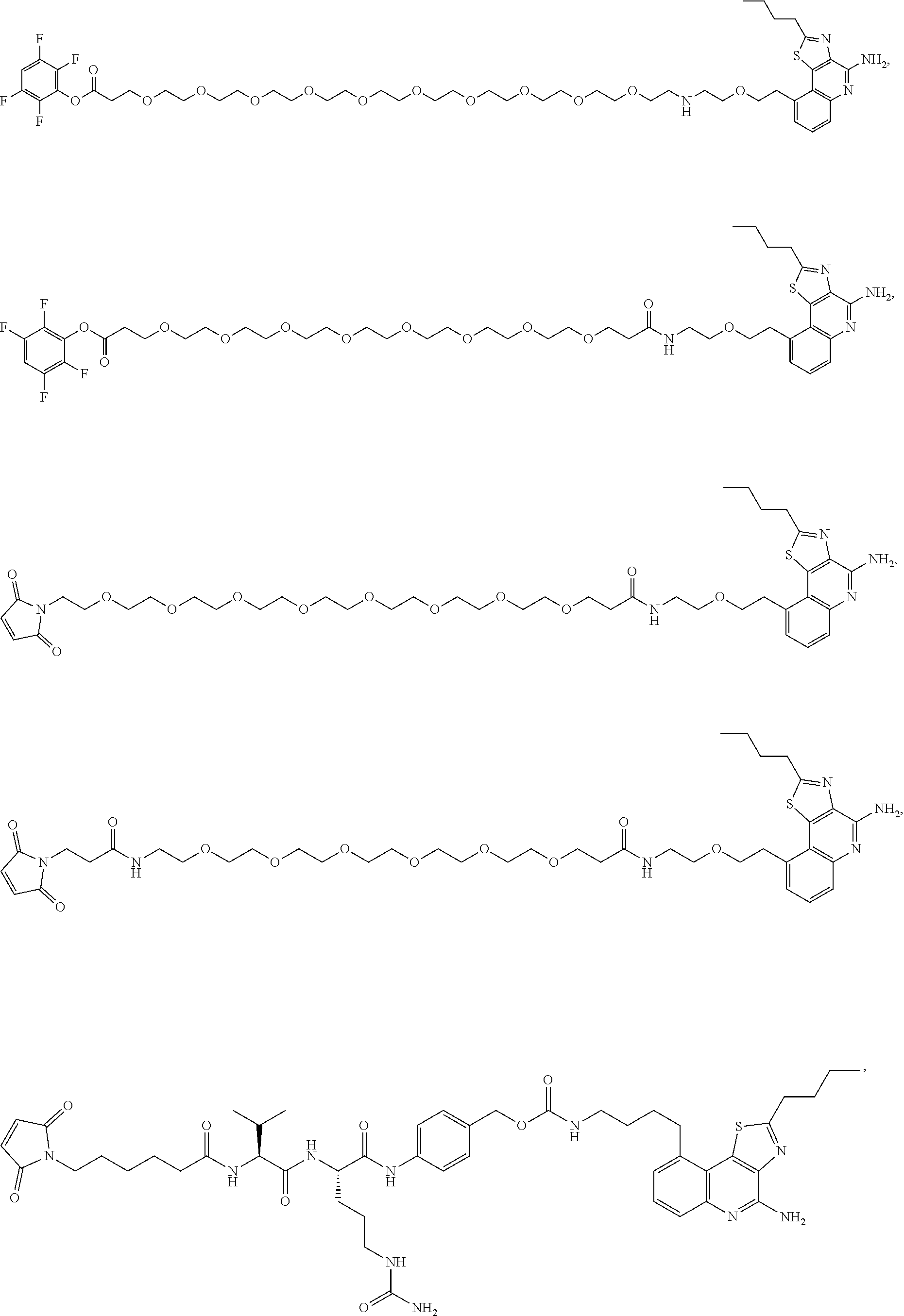 [SMILES: CCCCc1nc2c(N)nc3cccc(CCOCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCOCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCOCCNC(=O)CCOCCOCCOCCOCCOCCOCCOCCOCCN4C(=O)C=CC4=O)c3c2s1 ; CCCCc1nc2c(N)nc3cccc(CCOCCNC(=O)CCOCCOCCOCCOCCOCCOCCNC(=O)CCN4C(=O)C=CC4=O)c3c2s1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1]

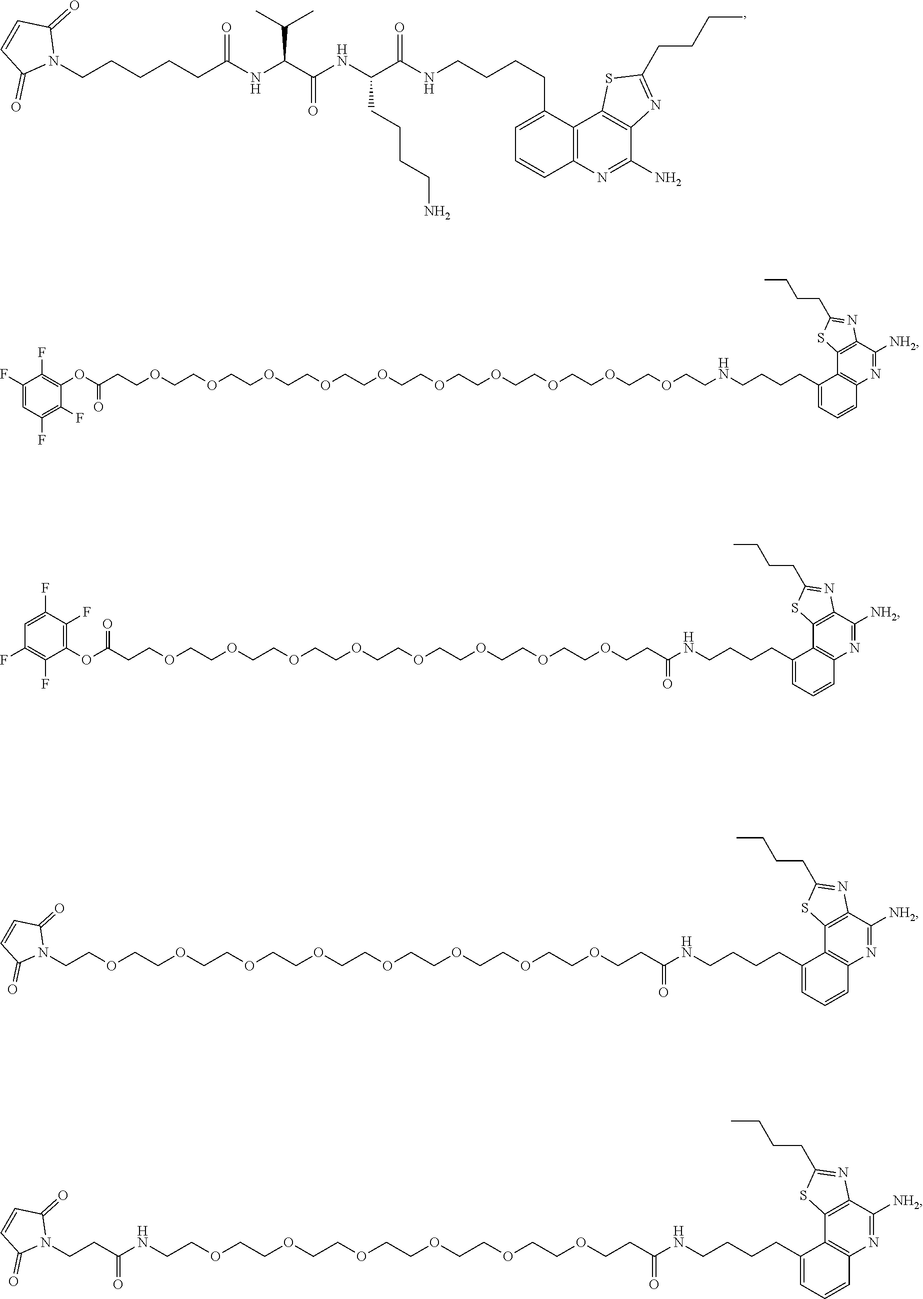 [SMILES: CCCCc1nc2c(N)nc3cccc(CCCCNC(=O)[C@H](CCCCN)NC(=O)[C@@H](NC(=O)CCCCCN4C(=O)C=CC4=O)C(C)C)c3c2s1 ; CCCCc1nc2c(N)nc3cccc(CCCCNCCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCNC(=O)CCOCCOCCOCCOCCOCCOCCOCCOCCN4C(=O)C=CC4=O)c3c2s1 ; CCCCc1nc2c(N)nc3cccc(CCCCNC(=O)CCOCCOCCOCCOCCOCCOCCNC(=O)CCN4C(=O)C=CC4=O)c3c2s1]

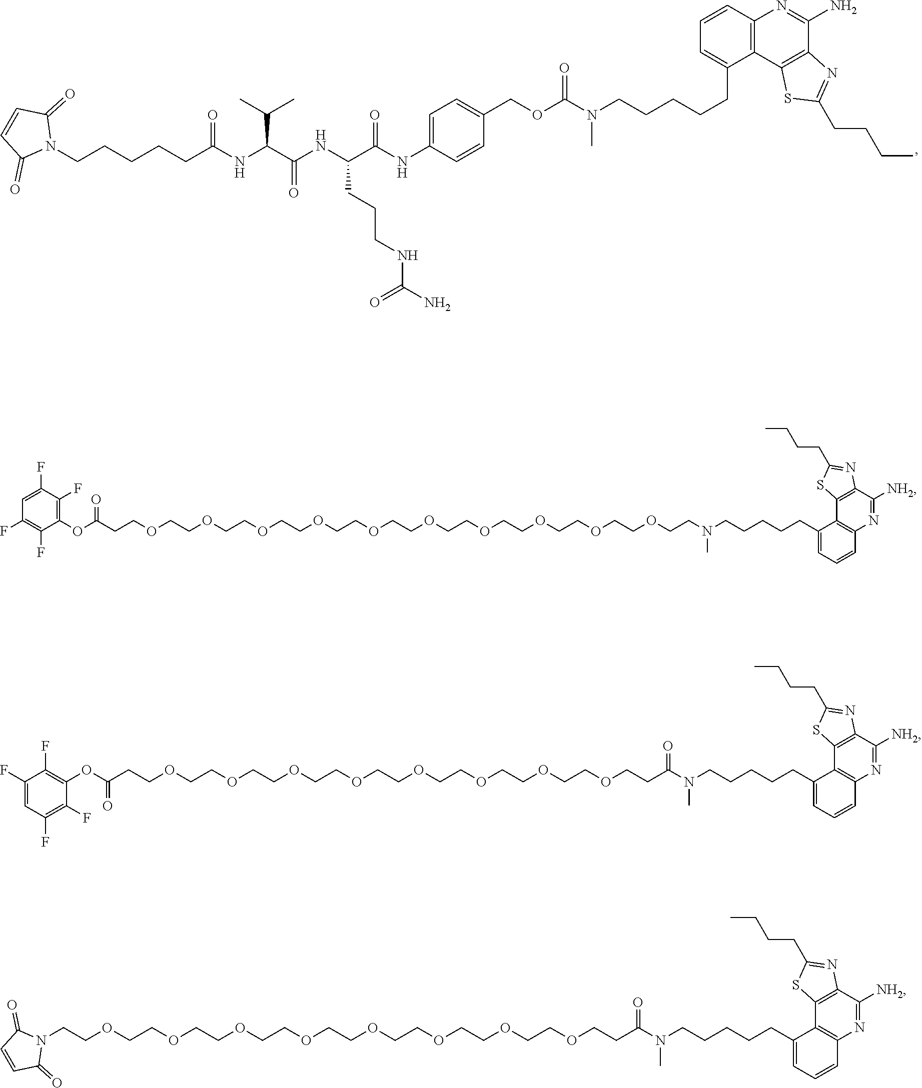 [SMILES: CCCCc1nc2c(N)nc3cccc(CCCCCN(C)C(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCN(C)CCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCCN(C)C(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCN(C)C(=O)CCOCCOCCOCCOCCOCCOCCOCCOCCN4C(=O)C=CC4=O)c3c2s1]

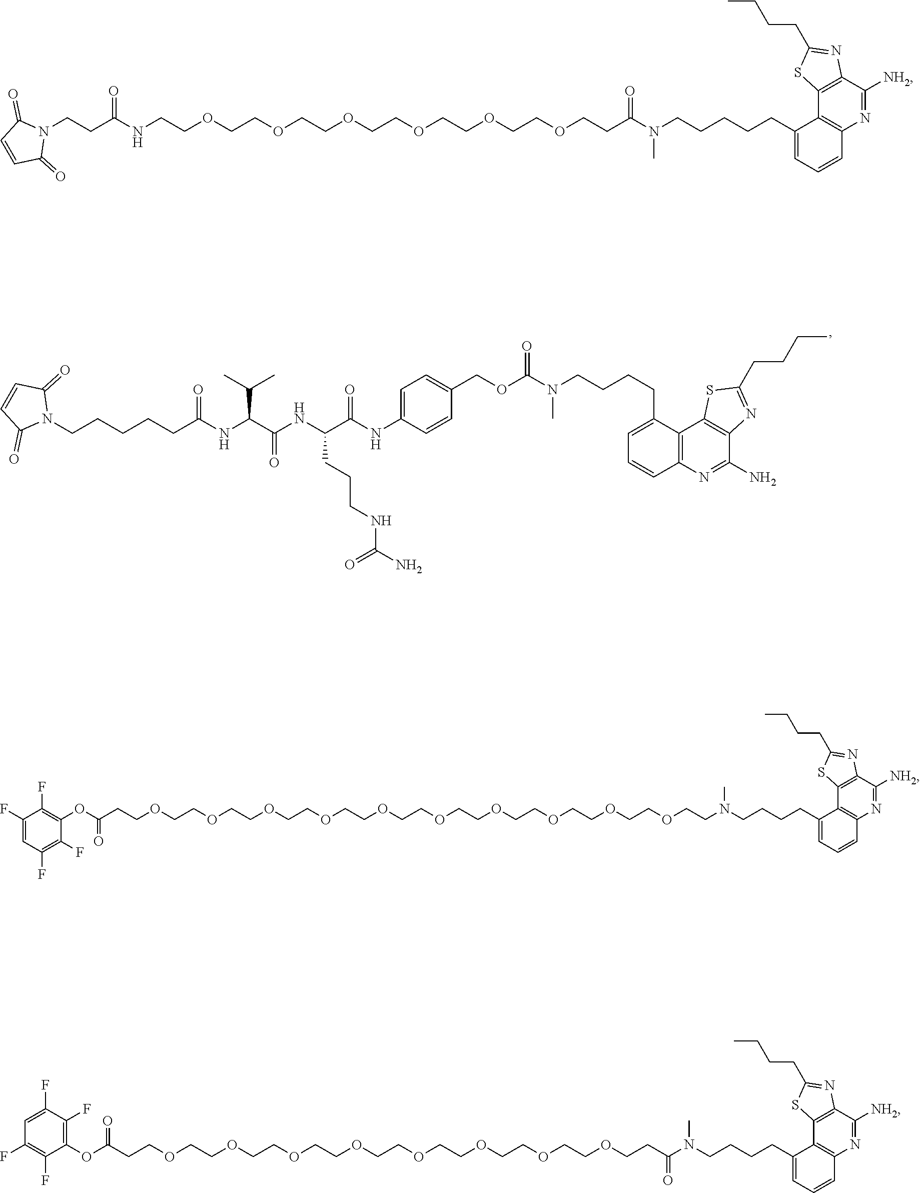 [SMILES: CCCCc1nc2c(N)nc3cccc(CCCCCN(C)C(=O)CCOCCOCCOCCOCCOCCOCCNC(=O)CCN4C(=O)C=CC4=O)c3c2s1 ; CCCCc1nc2c(N)nc3cccc(CCCCN(C)C(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCN(C)CCOCCOCCOCCOCCOCCOCCOCCOCCOCCOCCC(=O)Oc4c(F)c(F)cc(F)c4F)c3c2s1 ; CCCCc1nc2c(N)nc3cccc(CCCCN(C)C(=O)CCOCCOCCOCCOCCOCCOCCOCCOCCC(=O)Oc4c(F)c(F)cc(F)c4F)c3c2s1]

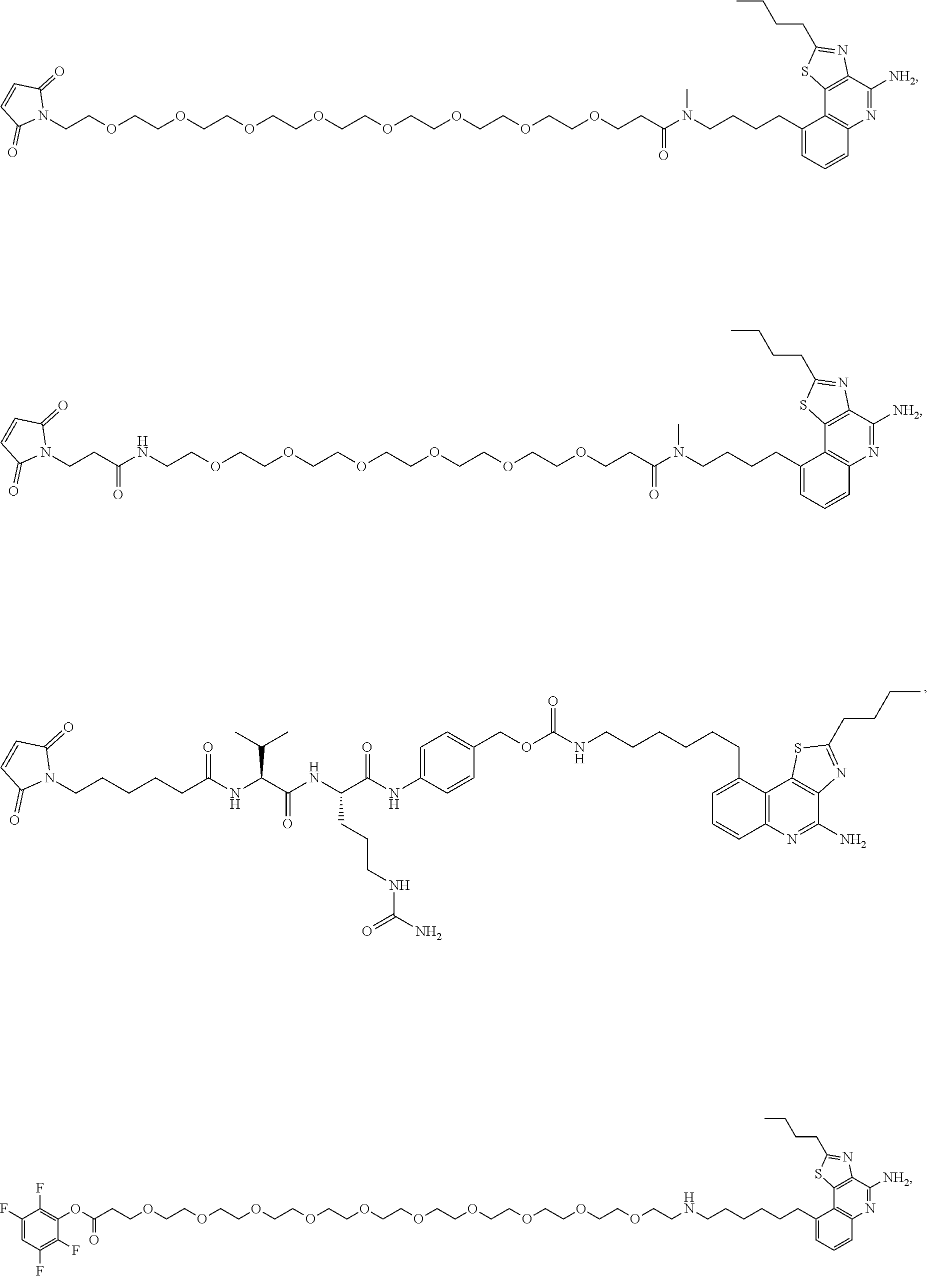 [SMILES: CCCCc1nc2c(N)nc3cccc(CCCCN(C)C(=O)CCOCCOCCOCCOCCOCCOCCOCCOCCN4C(=O)C=CC4=O)c3c2s1 ; CCCCc1nc2c(N)nc3cccc(CCCCN(C)C(=O)CCOCCOCCOCCOCCOCCOCCNC(=O)CCN4C(=O)C=CC4=O)c3c2s1 ; CCCCc1nc2c(N)nc3cccc(CCCCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2s1 ; CCCCc1nc2c(N)nc3cccc(CCCCCCNCCOCCOCCOCCOCCOCCOCCOCCOCCOCCOCCC(=O)Oc4c(F)c(F)cc(F)c4F)c3c2s1]

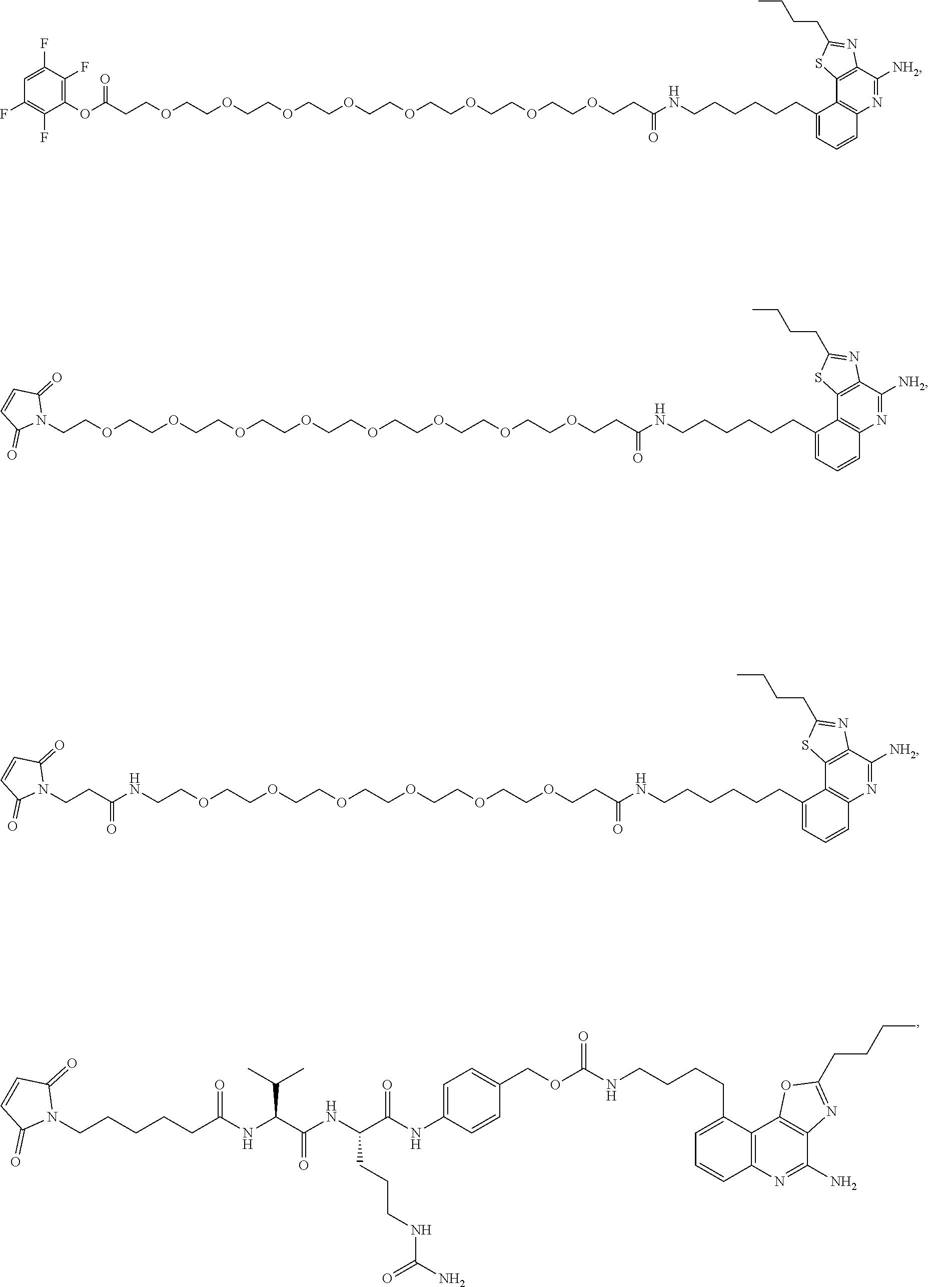 [SMILES: CCCCc1nc2c(N)nc3cccc(CCCCCCNC(=O)CCOCCOCCOCCOCCOCCOCCOCCOCCC(=O)Oc4c(F)c(F)cc(F)c4F)c3c2s1 ; CCCCc1nc2c(N)nc3cccc(CCCCCCNC(=O)CCOCCOCCOCCOCCOCCOCCOCCOCCN4C(=O)C=CC4=O)c3c2s1 ; CCCCc1nc2c(N)nc3cccc(CCCCCCNC(=O)CCOCCOCCOCCOCCOCCOCCNC(=O)CCN4C(=O)C=CC4=O)c3c2s1 ; CCCCc1nc2c(N)nc3cccc(CCCCNC(=O)OCc4ccc(NC(=O)[C@H](CCCNC(N)=O)NC(=O)[C@@H](NC(=O)CCCCCN5C(=O)C=CC5=O)C(C)C)cc4)c3c2o1]

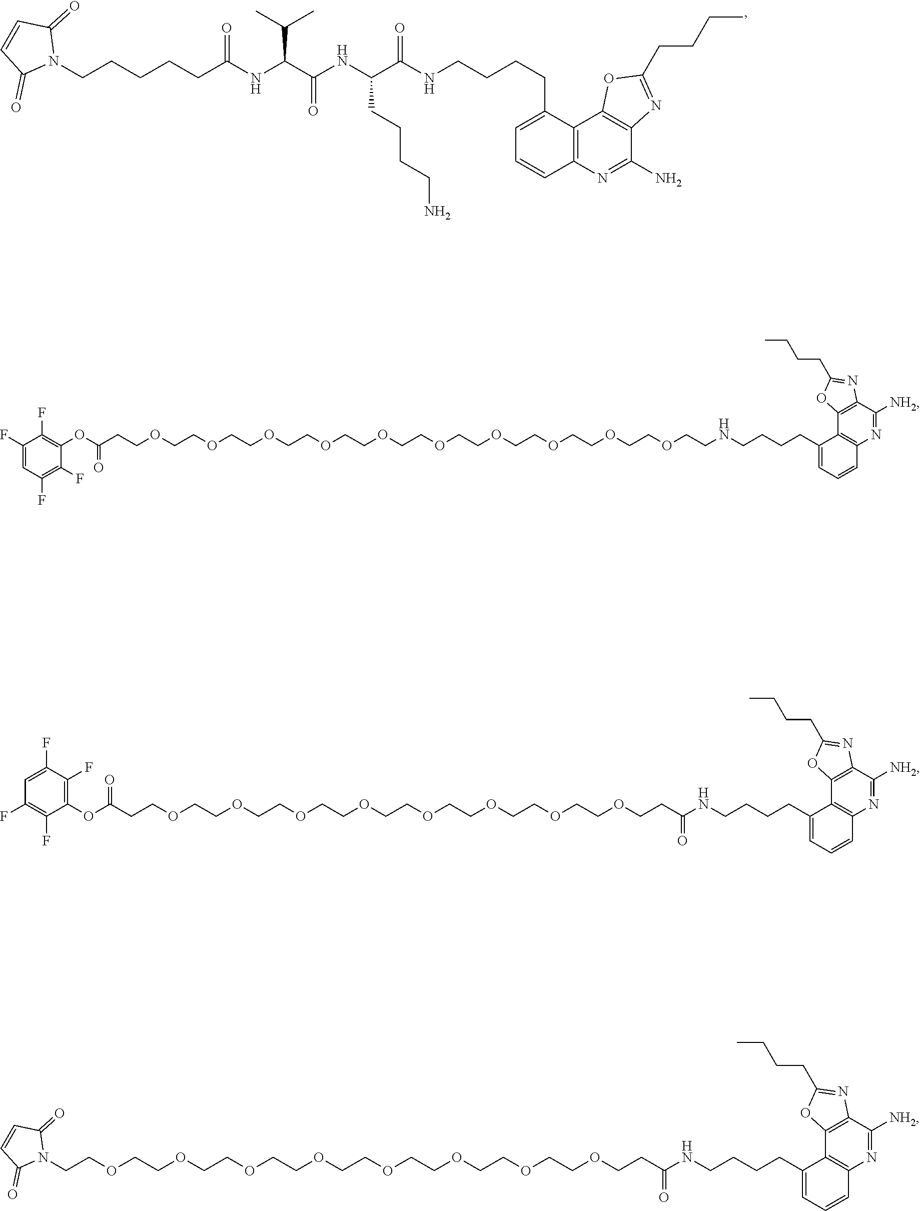 [SMILES: CCCCc1nc2c(N)nc3cccc(CCCCNC(=O)[C@H](CCCCN)NC(=O)[C@@H](NC(=O)CCCCCN4C(=O)C=CC4=O)C(C)C)c3c2o1 ; CCCCc1nc2c(N)nc3cccc(CCCCNCCOCCOCCOCCOCCOCCOCCOCCOCCOCCOCCC(=O)Oc4c(F)c(F)cc(F)c4F)c3c2o1 ; CCCCc1nc2c(N)nc3cccc(CCCCNC(=O)CCOCCOCCOCCOCCOCCOCCOCCOCCC(=O)Oc4c(F)c(F)cc(F)c4F)c3c2o1 ; CCCCc1nc2c(N)nc3cccc(CCCCNC(=O)CCOCCOCCOCCOCCOCCOCCOCCOCCN4C(=O)C=CC4=O)c3c2o1]

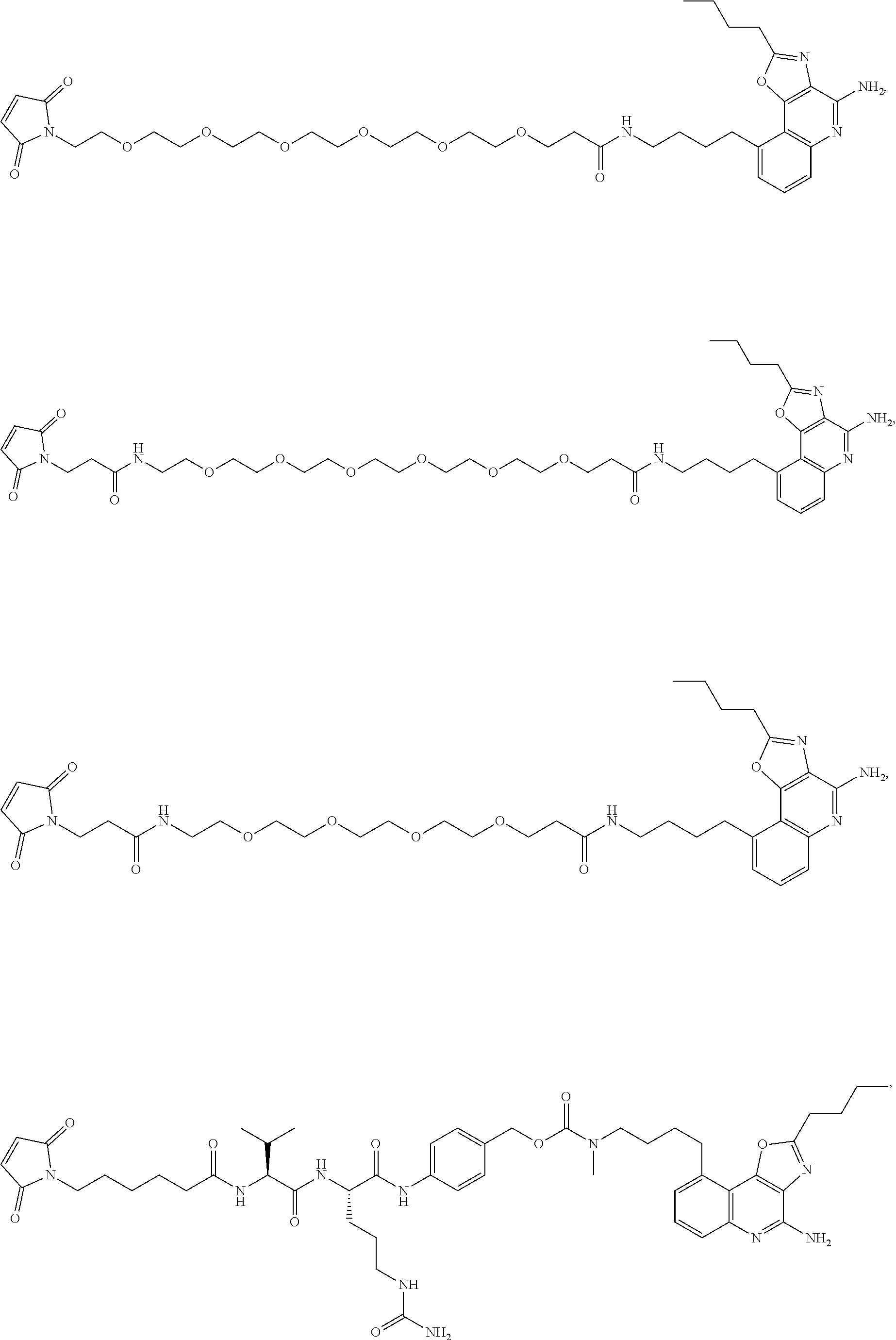 [SMILES: CCCCc1nc2c(N)nc3cccc(CCCCNC(=O)CCOCCOCCOCCOCCOCCOCCN4C(=O)C=CC4=O)c3c2o1 ; CCCCc1nc2c(N)nc3cccc(CCCCNC(=O)CCOCCOCCOCCOCCOCCOCCNC(=O)CCN4C(=O)C=CC4=O)c3c2o1 ; CCCCc1nc2c(N)nc3cccc(CCCCNC(=O)CCOCCOCCOCCOCCNC(=O)CCN4C(=O)C=CC4=O)c3c2o1 ; CCCCc1nc2c(N)nc3cccc(CCCCN(C)C(=O)OCc4ccc(NC(=O)[C@H](CCCNC(N)=O)NC(=O)[C@@H](NC(=O)CCCCCN5C(=O)C=CC5=O)C(C)C)cc4)c3c2o1]

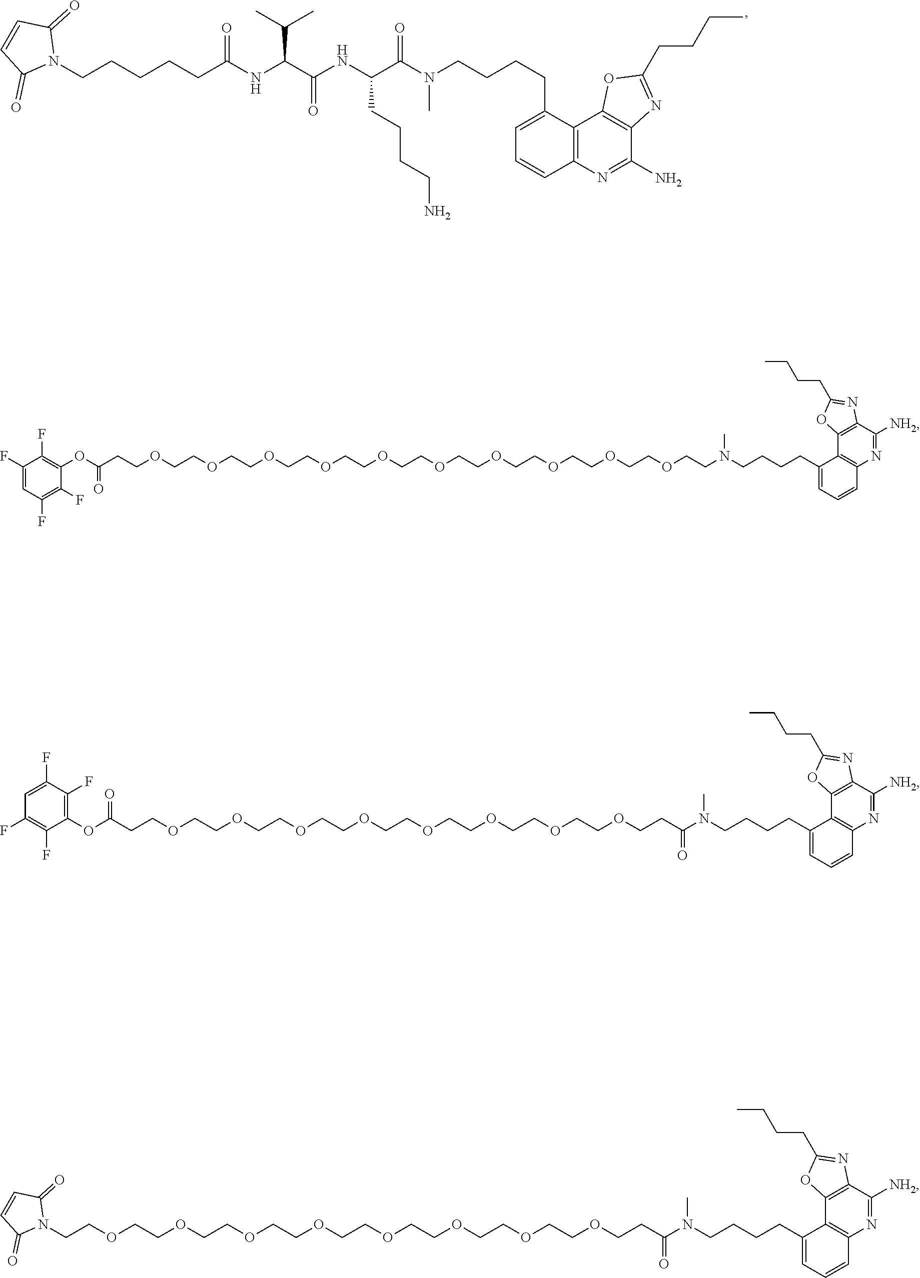 [SMILES: CCCCc1nc2c(N)nc3cccc(CCCCN(C)C(=O)[C@H](CCCCN)NC(=O)[C@@H](NC(=O)CCCCCN4C(=O)C=CC4=O)C(C)C)c3c2o1 ; CCCCc1nc2c(N)nc3cccc(CCCCN(C)CCOCCOCCOCCOCCOCCOCCOCCOCCOCCOCCC(=O)Oc4c(F)c(F)cc(F)c4F)c3c2o1 ; CCCCc1nc2c(N)nc3cccc(CCCCN(C)C(=O)CCOCCOCCOCCOCCOCCOCCOCCOCCC(=O)Oc4c(F)c(F)cc(F)c4F)c3c2o1 ; CCCCc1nc2c(N)nc3cccc(CCCCN(C)C(=O)CCOCCOCCOCCOCCOCCOCCOCCOCCN4C(=O)C=CC4=O)c3c2o1]

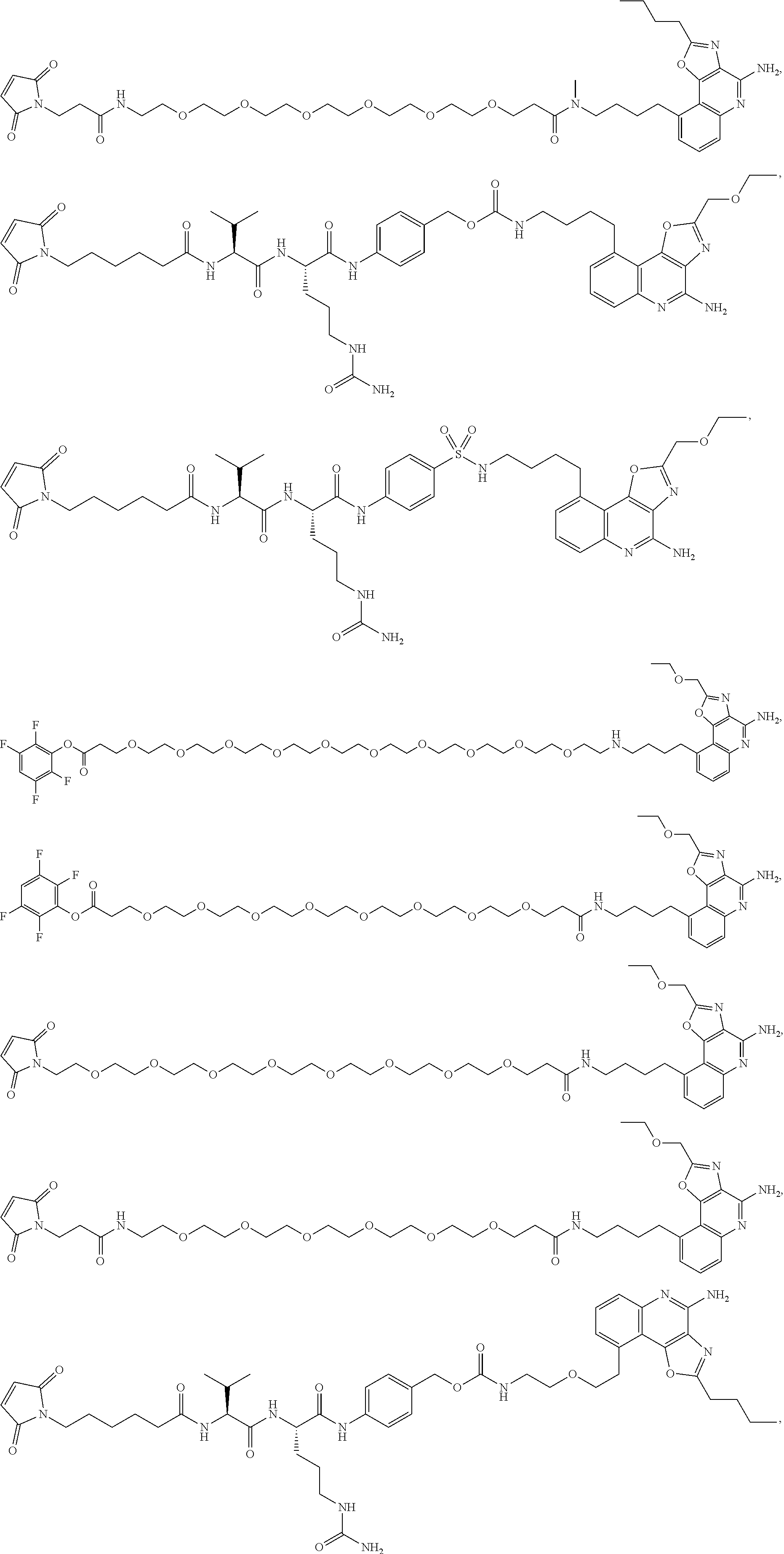

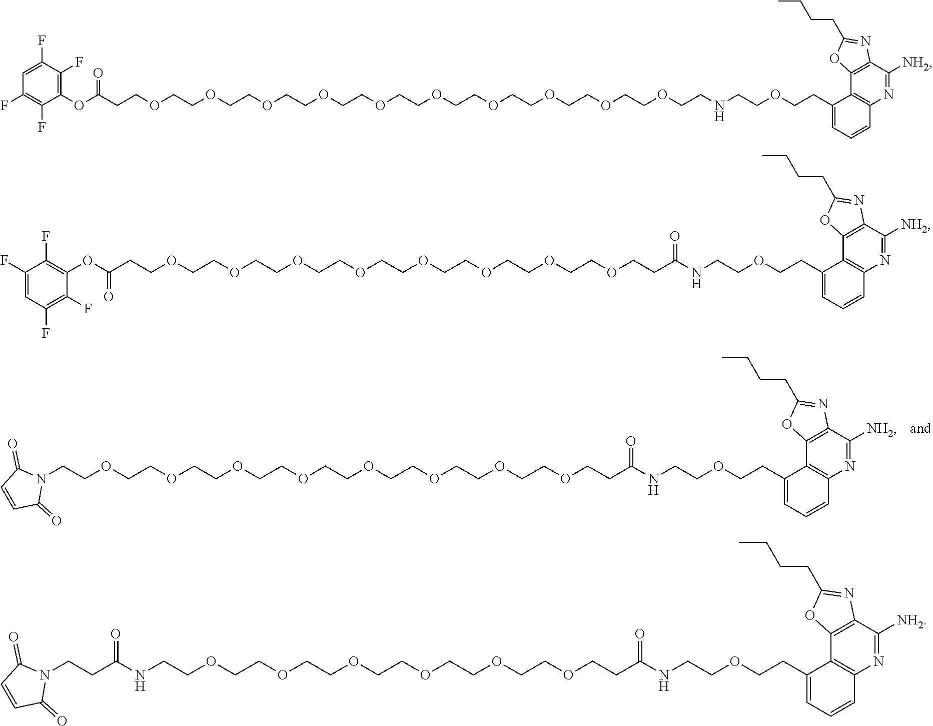 [SMILES: CCCCc1nc2c(N)nc3cccc(CCOCCNCCOCCOCCOCCOCCOCCOCCOCCOCCOCCOCCC(=O)Oc4c(F)c(F)cc(F)c4F)c3c2o1 ; CCCCc1nc2c(N)nc3cccc(CCOCCNC(=O)CCOCCOCCOCCOCCOCCOCCOCCOCCC(=O)Oc4c(F)c(F)cc(F)c4F)c3c2o1 ; CCCCc1nc2c(N)nc3cccc(CCOCCNC(=O)CCOCCOCCOCCOCCOCCOCCOCCOCCN4C(=O)C=CC4=O)c3c2o1 ; CCCCc1nc2c(N)nc3cccc(CCOCCNC(=O)CCOCCOCCOCCOCCOCCOCCNC(=O)CCN4C(=O)C=CC4=O)c3c2o1]

69. The compound according to claim 37, being a compound of formula (A-1), or a tautomer, an enantiomer or a diastereoisomer thereof, or a mixture of isomers thereof, or a pharmaceutically acceptable salt or solvate thereof: 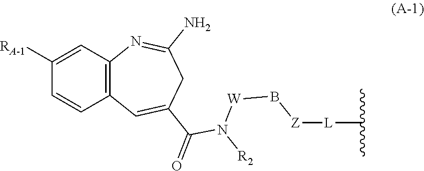
wherein
R_{A-1} is selected from R_{A2-1}-Lx-;
Lx is selected from a single bond, C₁-C₆ alkylene, —O—, —S—, —C(O)—, —NH—, —C(O)NH—, —NHC(O)—, —C(O)—N(C₁-C₆ alkyl)-, or —N(C₁-C₆ alkylene)-C(O)—;
R_{A2-1} is aryl, heteroaryl, or heterocyclyl, preferably phenyl, pyridinyl, or isobenzofuranonyl, and R_{A2-1} is optionally substituted with one or more substituents R_{A2-2}, wherein
each R_{A2-2} is independently selected from —C₁-C₆ alkyl, amino, —NH(C₁-C₆ alkyl), —N(C₁-C₆ alkyl)₂, —C₁-C₆ alkylamino, —C₁-C₆ alkyl-NH(C₁-C₆ alkyl), —C₁-C₆ alkyl-N(C₁-C₆ alkyl)₂, hydroxy, —C₁-C₆ alkoxy, —C₁-C₆ alkylhydroxy, —C₁-C₆ alkyl-C₁-C₆ alkoxy, halogen, halogenated C₁-C₆ alkyl, halogenated C₁-C₆ alkoxy, or —C(O)NRₑR_{f}, wherein Rₑ and R_{f} are each independently selected from H or C₁-C₆ alkyl, or Rₑ and R_{f}, together with the nitrogen atom to which they are connected, form a 5- or 6-membered nitrogen-containing heterocyclyl, such as tetrahydropyrrolyl or piperidinyl, the 5- or 6-membered nitrogen-containing heterocyclyl being optionally substituted with one or more substituents independently selected from C₁-C₆ alkyl, C₁-C₆ alkoxy, hydroxy, oxo, amino, —NH(C₁-C₆alkyl), or —N(C₁-C₆ alkyl)₂;
R₂ is selected from —C₁-C₆ alkyl, —C₁-C₆ alkoxy, —C₁-C₆ alkylenehydroxy, or halogenated C₁-C₆ alkyl;
W is selected from —C₁-C₆ alkylene- and —O—C₁-C₆ alkylene-;
B is phenyl, and is optionally substituted with one or more substituents selected from: halogen, C₁-C₆ alkyl, halogenated C₁-C₆ alkyl, and C₁-C₆ alkoxy; and
Z is —NH(R_{Z-1}), R_{Z-1} being selected from H, —C₁-C₆ alkyl, or halogenated C₁-C₆ alkyl;
L is a linker unit, preferably the linker unit as defined in any one of claims 26-30; the wavy line represents being linked to the ligand via the nitrogen atom or the carbon atom on the L group.

70. The compound according to claim 69, wherein the linker unit L is selected from:

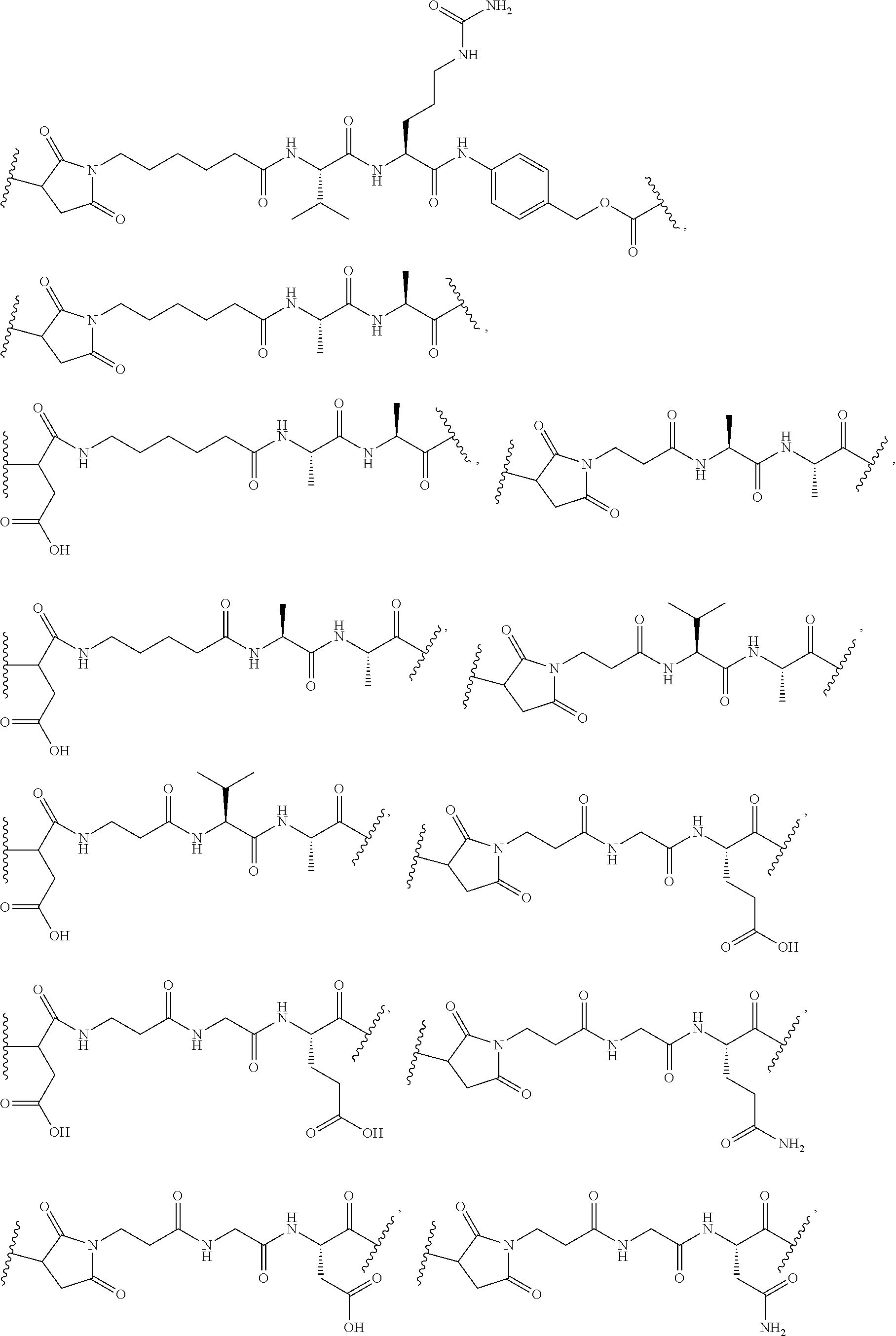

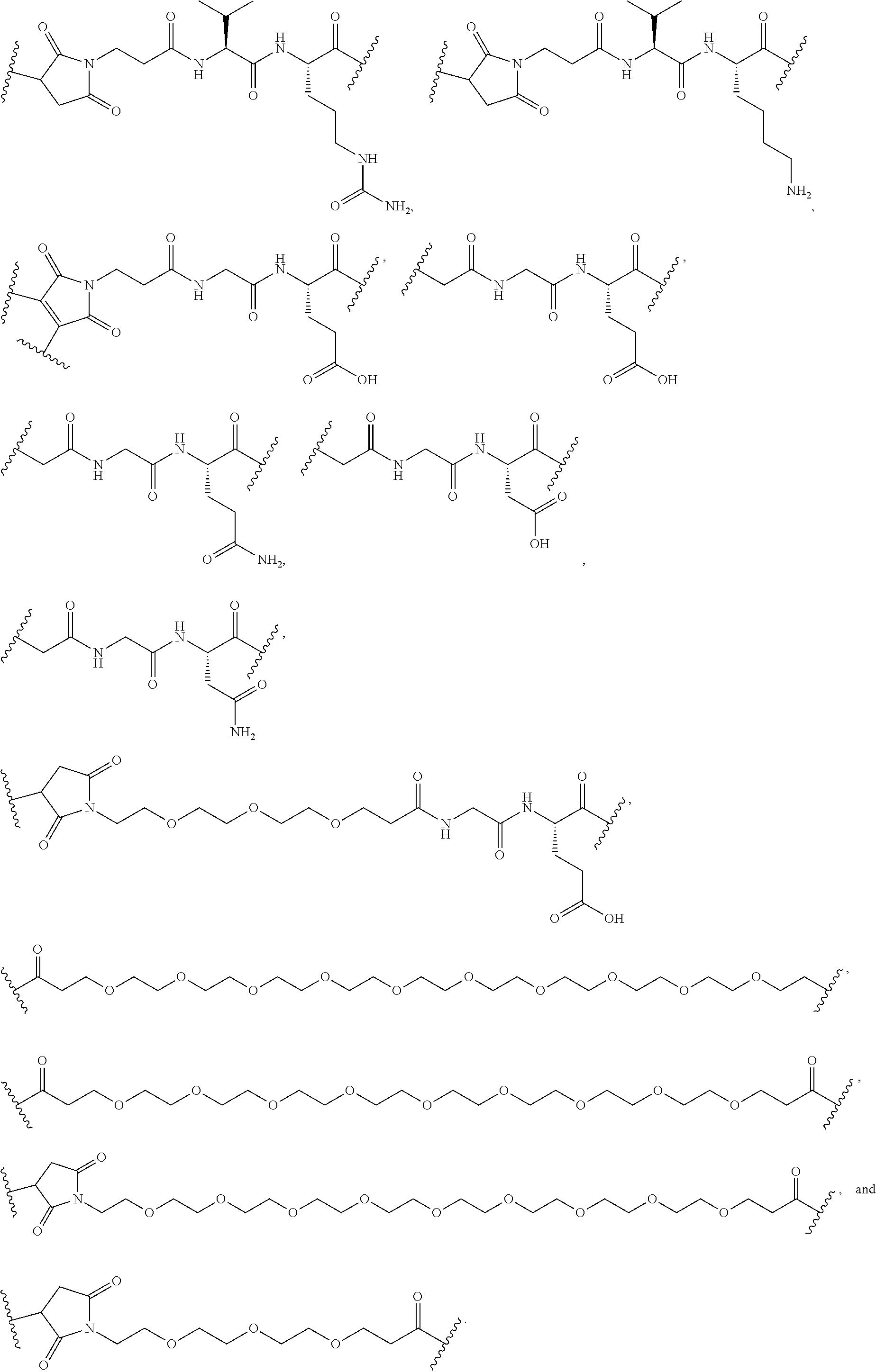

71. A compound, selected from the following compounds, or tautomers, enantiomers or diastereoisomers thereof, or mixtures of isomers thereof, or pharmaceutically acceptable salts or solvates thereof:

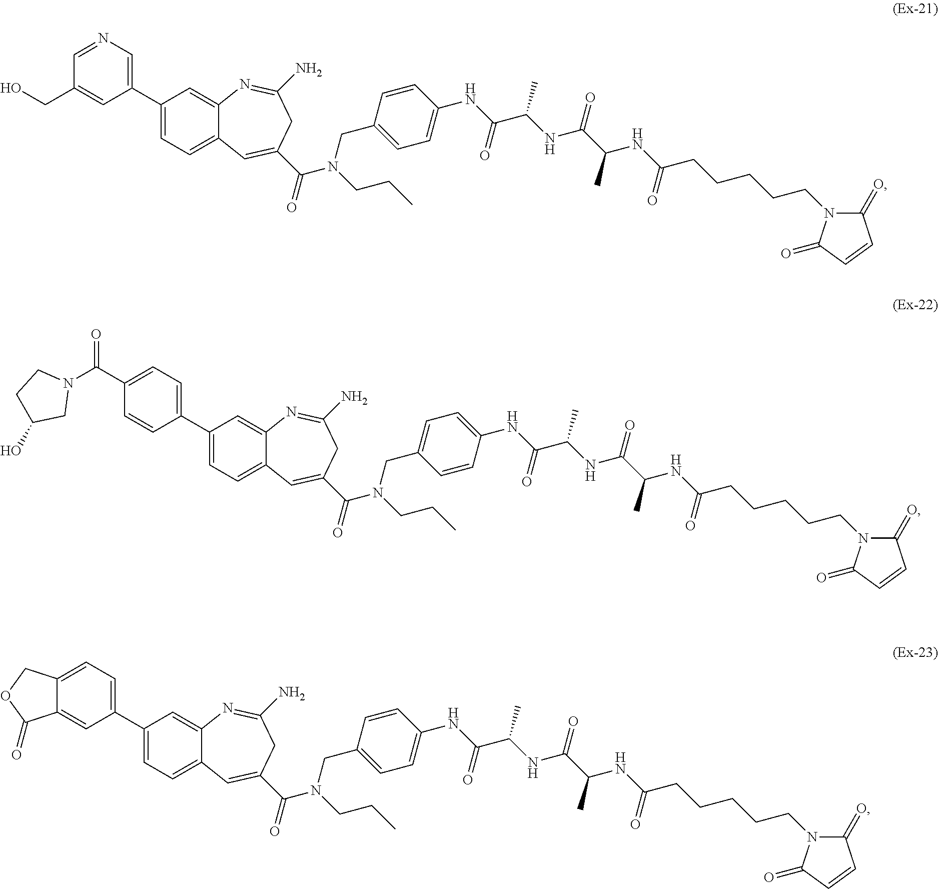 [SMILES: CCCN(Cc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc(C(=O)N4CC[C@@H](O)C4)cc3)cc2N=C(N)C1 ; CCCN(Cc1ccc(NC(=O)[C@H](C)NC(=O)[C@H](C)NC(=O)CCCCCN2C(=O)C=CC2=O)cc1)C(=O)C1=Cc2ccc(-c3ccc4c(c3)C(=O)OC4)cc2N=C(N)C1]

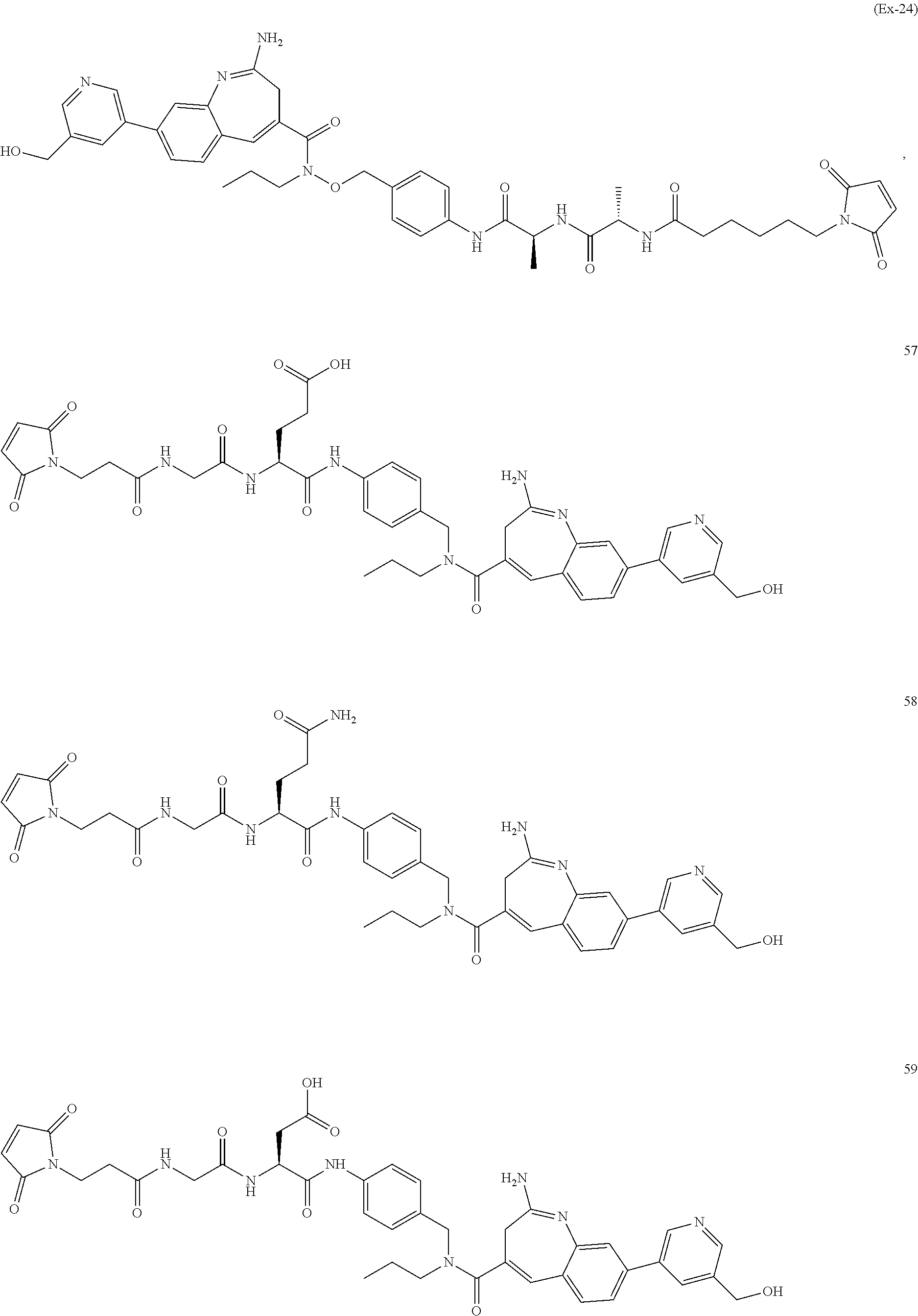 [SMILES: CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(=O)O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CCC(N)=O)NC(=O)CNC(=O)CCN2C(=O)C=CC2=O)cc1)C(=O)C1=Cc2ccc(-c3cncc(CO)c3)cc2N=C(N)C1 ; CCCN(Cc1ccc(NC(=O)[C@H](CC(=O)O)NC(=O)CNC(=O)CCN2C(=O)C=CC2=O)cc1)C(=O)C1=Cc2ccc(-c3cncc(CO)c3)cc2N=C(N)C1]

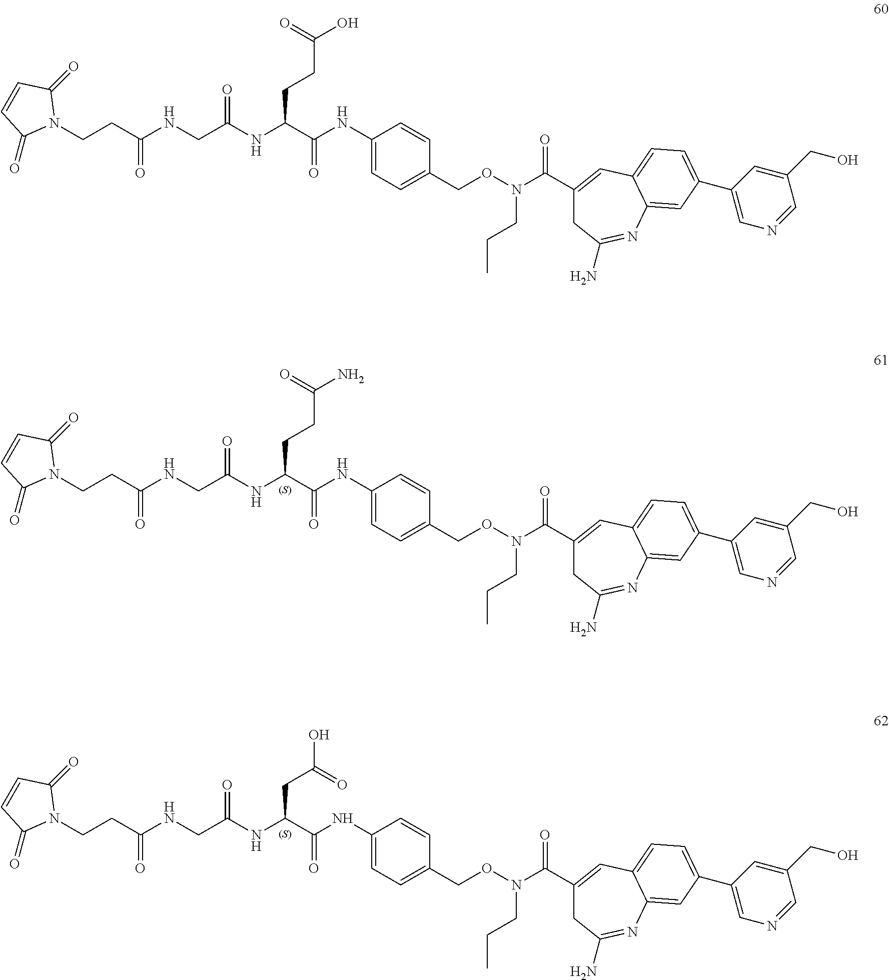

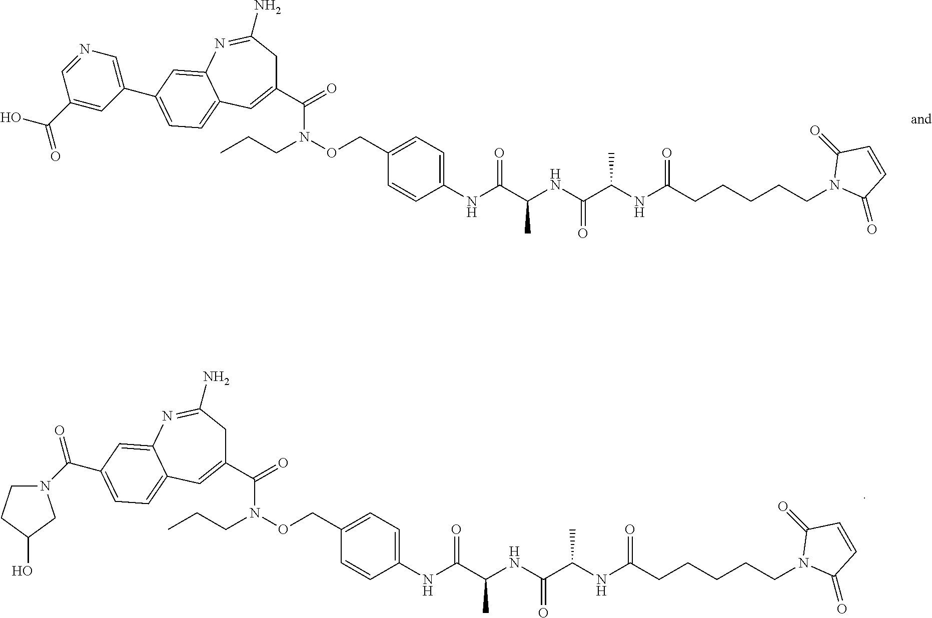 [SMILES: CCCN(OCc1ccc(NC(=O)[C@H](C)NC(=O)[C@H](C)NC(=O)CCCCCN2C(=O)C=CC2=O)cc1)C(=O)C1=Cc2ccc(-c3cncc(C(=O)O)c3)cc2N=C(N)C1 ; CCCN(OCc1ccc(NC(=O)[C@H](C)NC(=O)[C@H](C)NC(=O)CCCCCN2C(=O)C=CC2=O)cc1)C(=O)C1=Cc2ccc(C(=O)N3CCC(O)C3)cc2N=C(N)C1]

72. The ligand-drug conjugate according to claim 3, being a ligand-drug conjugate of formula (A-2), or a tautomer, an enantiomer or a diastereoisomer thereof, or a mixture of isomers thereof, or a pharmaceutically acceptable salt or solvate thereof: 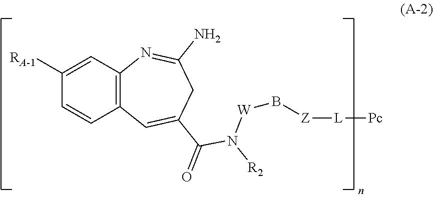
wherein R_{A-1}, W, B, Z, and R₂ are as defined in claim 69; L is the linker unit as defined in claim 70;
n is an integer or a decimal from 1 to 10, preferably an integer or a decimal from 2 to 8; Pc is a ligand, preferably an antibody or an antigen-binding fragment thereof.

73. The ligand-drug conjugate according to claim 72, wherein Pc is an antibody or an antigen-binding fragment thereof that targets GPC3, Trop2, and HER2; preferably, the antibody is selected from: trastuzumab, pertuzumab, sacituzumab, and codrituzumab.

74. A pharmaceutical composition, comprising the ligand-drug conjugate, or the isomer thereof, or the mixture thereof, or the pharmaceutically acceptable salt thereof according to any one of claims 1-36 or 40-65, and a pharmaceutically acceptable carrier or excipient, or comprising the compound or the ligand-drug conjugate, or the tautomer, the enantiomer or the diastereoisomer thereof, or the mixture of isomers thereof, or the pharmaceutically acceptable salt or solvate thereof according to any one of claims 69-73.

75. A method for modulating immune system functionality, comprising administering the ligand-drug conjugate, or the isomer thereof, or the mixture thereof, or the pharmaceutically acceptable salt thereof according to any one of claims 1-36 or 40-65, or administering the compound or the ligand-drug conjugate, or the tautomer, the enantiomer or the diastereoisomer thereof, or the mixture of isomers thereof, or the pharmaceutically acceptable salt or solvate thereof according to any one of claims 69-73, or administering the pharmaceutical composition according to claim 74.

76. Use of the ligand-drug conjugate, or the isomer thereof, or the mixture thereof, or the pharmaceutically acceptable salt thereof according to any one of claims 1-36 or 40-65, or the compound or the ligand-drug conjugate, or the tautomer, the enantiomer or the diastereoisomer thereof, or the mixture of isomers thereof, or the pharmaceutically acceptable salt or solvate thereof according to any one of claims 69-73, or the pharmaceutical composition according to claim 74 in the preparation of a medicament for preventing and/or treating a disease and/or a symptom.

77. The use according to claim 76, wherein the disease and/or the symptom comprises a disease and/or a symptom associated with Toll-like receptor (TLR) signaling.

78. The use according to claim 76 or 77, wherein the disease and/or the symptom is selected from the group consisting of: a tumor, an autoimmune disease, an inflammation, sepsis, an allergy, asthma, transplant rejection, graft-versus-host disease, immunodeficiency, and an infection caused by a virus.

79. The use according to any one of claims 76-78, wherein the disease and/or the symptom is selected from the group consisting of: melanoma, lung cancer, liver cancer, basal cell carcinoma, kidney cancer, myeloma, biliary tract cancer, brain cancer, breast cancer, cervical cancer, choriocarcinoma, colon cancer, rectal cancer, head and neck cancer, peritoneal tumor, fallopian tube cancer, endometrial cancer, esophageal cancer, gastric cancer, leukemia, lymphoma, sarcoma, neuroblastoma, oral cancer, ovarian cancer, pancreatic cancer, prostate cancer, testicular cancer, skin cancer, and thyroid cancer.

80. The use according to any one of claims 76-78, wherein the disease and/or the symptom is an infection caused by a virus selected from the group consisting of: dengue virus, yellow fever virus, West Nile virus, Japanese encephalitis virus, tick-borne encephalitis virus, Kunjin virus, Murray Valley encephalitis virus, St. Louis encephalitis virus, Omsk hemorrhagic fever virus, bovine viral diarrhea virus, Zika virus, HIV (human immunodeficiency virus), HBV (hepatitis B virus), HCV (hepatitis C virus), HPV (human papillomavirus), RSV (respiratory syncytial virus), SARS-CoV (severe acute respiratory syndrome coronavirus), SARS-CoV-2 (severe acute respiratory syndrome coronavirus 2), MERS-CoV (middle east respiratory syndrome coronavirus), and influenza virus.


SEQUENCE LISTING (40 sequences)

SEQ ID NO: 1 - protein, 11 aa, synthetic construct
RASQDVNTAVA

SEQ ID NO: 2 - protein, 11 aa, synthetic construct
KASQDVSIGVA

SEQ ID NO: 3 - protein, 11 aa, synthetic construct
KASQDVSIAVA

SEQ ID NO: 4 - protein, 16 aa, synthetic construct
RSSQSLVHSNRNTYLH

SEQ ID NO: 5 - protein, 6 aa, synthetic construct
SASFLY

SEQ ID NO: 6 - protein, 7 aa, synthetic construct
SASYRYT

SEQ ID NO: 7 - protein, 7 aa, synthetic construct
SASYRYT

SEQ ID NO: 8 - protein, 7 aa, synthetic construct
KVSNRFS

SEQ ID NO: 9 - protein, 8 aa, synthetic construct
QQHYTTPP

SEQ ID NO: 10 - protein, 8 aa, synthetic construct
QQYYIYPY

SEQ ID NO: 11 - protein, 9 aa, synthetic construct
QQHYITPLT

SEQ ID NO: 12 - protein, 9 aa, synthetic construct
SQNTHVPPT

SEQ ID NO: 13 - protein, 10 aa, synthetic construct
GFNIKDTYIH

SEQ ID NO: 14 - protein, 10 aa, synthetic construct
GFTFTDYTMD

SEQ ID NO: 15 - protein, 5 aa, synthetic construct
NYGMN

SEQ ID NO: 16 - protein, 5 aa, synthetic construct
DYEMH

SEQ ID NO: 17 - protein, 18 aa, synthetic construct
ARIYPTNGYTRYADSVKG

SEQ ID NO: 18 - protein, 18 aa, synthetic construct
ADVNPNSGGSIYNQRFKG

SEQ ID NO: 19 - protein, 17 aa, synthetic construct
WINTYTGEPTYTDDFKG

SEQ ID NO: 20 - protein, 17 aa, synthetic construct
ALDPKTGDTAYSQKFKG

SEQ ID NO: 21 - protein, 13 aa, synthetic construct
SRWGGDGFYAMDY

SEQ ID NO: 22 - protein, 12 aa, synthetic construct
ARNLGPSFYFDY

SEQ ID NO: 23 - protein, 12 aa, synthetic construct
GGFGSSYWYFDV

SEQ ID NO: 24 - protein, 6 aa, synthetic construct
FYSYTY

SEQ ID NO: 25 - protein, 107 aa, synthetic construct [sequence omitted]

SEQ ID NO: 26 - protein, 107 aa, synthetic construct [sequence omitted]

SEQ ID NO: 27 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 28 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 29 - protein, 120 aa, synthetic construct [sequence omitted]

SEQ ID NO: 30 - protein, 119 aa, synthetic construct [sequence omitted]

SEQ ID NO: 31 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 32 - protein, 115 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 33 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 219 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 445 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK